(19) United States
(12) Patent Application Publication
(10) Pub. No.: US 2023/0053540 A1
(43) Pub. Date: Feb. 23, 2023

(54) TREATMENT OF LIVER INJURY

(71) Applicants: Massachusetts Institute of Technology, Cambridge, MA (US); The Brigham and Women's Hospital, Inc., Boston, MA (US); Dana-Farber Cancer Institute, Inc., Boston, MA (US)

(72) Inventors: Alexander K. Shalek, Cambridge, MA (US); Kellie Elizabeth Kolb, Cambridge, MA (US); Chad Walesky, Boston, MA (US); Wolfram Goessling, Boston, MA (US)

(21) Appl. No.: 17/431,835

(22) PCT Filed: Feb. 19, 2020
(86) PCT No.: PCT/US2020/018921

§ 371 Date: Aug. 18, 2021

Related U.S. Application Data
(60) Provisional application No. 62/807,569, filed on Feb. 19, 2019.
(60) Provisional application No. 62/925,693, filed on Oct. 24, 2019.

(51) Int. Cl. A61K 38/00; A61K 45/06; C12N 15/11; C12N 15/63; A61P 1/16

(52) U.S. Cl. CPC A61K 38/005; A61K 45/06; A61P 1/16; C12N 15/63; C12N 15/111

(57) ABSTRACT

A method of treating an injury in an organ or tissue includes administering to a subject in need thereof an agent that modulates expression and/or activity of one or more genes involved in the regeneration and functional compensation of the tissue or organ in response to the injury.

### CROSS REFERENCE TO RELATED APPLICATIONS

This application claims the benefit of U.S. Provisional Applications Nos. 62/925,693, filed Oct. 24, 2019, and 62/807,569, filed Feb. 19, 2019. The entire contents of the above-identified applications are hereby fully incorporated herein by reference.

### STATEMENT REGARDING FEDERALLY SPONSORED RESEARCH

This invention was made with government support under Grant Nos. GM119419 HG0061931, DA046277, DK111151, DK090311, DK105198, OD017870 awarded by the National Institutes of Health. The government has certain rights in the invention.

### TECHNICAL FIELD

The subject matter disclosed herein is generally directed to treating injuries in organs and tissues.

### BACKGROUND

The liver is a vital organ with a wide array of functions, including homeostasis of glucose, protein, and lipid metabolism, production of bile, synthesis of critical serum proteins, and metabolism of endogenous and xenobiotic toxins and toxicants. Because of its essential role in detoxification, the liver experiences frequent toxic insults leading to injury, cell death, and loss of functional cell mass. However, the liver has an unparalleled capacity to regenerate in order to maintain function. Under extreme stress, the regenerative capacity of the liver can be overwhelmed, leading to acute liver failure (ALF) and, ultimately, death.

### SUMMARY

In certain example embodiments, methods of treating liver injury are provided comprising stimulating functional compensation in liver cells by administering an agent that stimulates macrophage Wnt signaling. In an aspect, administering an agent comprises delivering a vector that targets liver macrophages, or comprises delivery of an agent targeting hepatocytes thereby stimulating macrophage Wnt signaling at the site of livery injury.

Methods of decreasing cancer susceptibility and/or inflammation are also provided comprising administering a subject in need thereof an inhibitor of peroxisome proliferator-activated receptors (PPARs), which may comprise a alpha, beta/delta or gamma PPAR.

In embodiments administration of the inhibitor is localized to the gut or localized to the liver. In an aspect, the subject treated is obese or on a high fat diet.

Methods of reducing risk of proliferation disorders or cancer in the liver comprising administering to a subject in need thereof an agent that increases expression of Sox9 or decreases expression of Lrg5 and Axin 2.

The present disclosure provides for methods and compositions for treating injuries in organs or tissues. In one aspect, the present disclosure provides a method of treating an injury in an organ or tissue, comprising administering to a subject in need thereof an agent that modulates expression and/or activity of one or more genes or gene products that have functions in regulation of proteolysis, chemical homeostasis, secretion by cells, regulation of hydrolase activity, regulation of body fluid levels, homeostatic process, wound healing, glycerolipid metabolic process, response to external stimuli, response to oxygen containing compounds, response to lipid, neutral lipid metabolic process, negative regulation of hydrolase activity, ion homeostasis, response to biotic stimulus, exocytosis, platelet degranulation, response to alcohol, regulated exocytosis, negative regulation of peptidase activity, extracellular matrix, secretory granule, platelet alpha granule, secretory granule lumen, secretory vesicle, vesicle lumen, blood microparticle, intracellular vesicle, extracellular space, cytoplasmic vesicle part, protein lipid complex, enzyme inhibitor activity, enzyme regulator activity, response to hypoxia, apoptosis, complement components functions and activities, or a combination thereof.

In some embodiments, the method further comprises administering to the subject in need thereof another agent that modulates expression and/or activity of one or more genes or gene products that have functions in PPAR signaling pathway, complement and/or coagulation cascades, PPARa activated gene expression, biological oxidations, metabolism of lipids and lipoproteins, nasopharyngeal carcinoma, intestine probiotics, plasma cell vs plasmablast, liver cancer, liver specific genes, multiple myeloma, response to UVb radiation, heart atrium vs ventricle, aging kidney no blood, endocrine therapy resistance, liver cancer, breast cancer basal, foxa2 targets, stem cell, lung cancer kras, tlx targets, liver cancer subclass g123, liver cancer subclass proliferation, liver cancer stem cell, liver cancer recurrence, liver cancer subclass s3, hepatoblastoma, liver development, liver hnf1a targets, matrisome, liver cancer krt19, fatty acid catabolic process, ammonium ion metabolic process, protein activation cascade, regulation of wound healing, response to estradiol, response to acid chemical, sterol homeostasis, lipoprotein metabolic process, fatty acid beta oxidation, protein maturation, regulation of locomotion, organic hydroxy compound metabolic process, organic acid biosynthetic process, monocarboxylic acid metabolic process, response to inorganic substance, regulation of vesicle mediated transport, regulation of fatty acid metabolic process, organic hydroxy compound transport, defense response, organophosphate ester transport, lipid homeostasis, secretion, anion transport, regulation of lipid biosynthetic process, response to xenobiotic stimulus, regulation of response to external stimulus, small molecule biosynthetic process, regulation of response to external stimulus, regulation of lipid metabolic process, amine metabolic process, autophagy, regulation of secretion, apoptotic signaling pathway, acute inflammatory response, regulation of catabolic process, maintenance of location, regulation of protein secretion, organic acid metabolic process, response to oxygen levels, regulation of cellular ketone metabolic process, organic acid catabolic process, regulation of response to wounding, regulation of extrinsic apoptotic signaling pathway, cellular lipid catabolic process, regulation of reactive oxygen species metabolic process, detoxification, regulation of peptidase activity, organic anion transport, inflammatory response, negative regulation of cell death, fatty acid metabolic process, lipid metabolic process, divalent inorganic cation homeostasis, regulation of endocytosis, alcohol metabolic process, immune response, cellular lipid metabolic process, monocarboxylic acid transport, negative regulation of apoptotic signaling pathway, multicellular organismal homeostasis, organic hydroxy compound biosynthetic process, regulation of cell death, lipid catabolic process, regulation of lipid metabolic process, regulation of steroid metabolic process, regulation of inflammatory response, response to toxic substance, cellular chemical homeostasis, regulation of transport, regulation of lipid catabolic process, regulation of immune effector process, lipid localization, regulation of proteolysis, regulation of secretion, regulation of response to wounding, regulation of multicellular organismal process, cellular homeostasis, single organism catabolic process, response to oxidative stress, behavior, acute phase response, regulation of response to external stimulus, regulation of apoptotic signaling pathway, regulation of cell proliferation, response to reactive oxygen species, endocytic vesicle, endoplasmic reticulum part, endoplasmic reticulum lumen, endoplasmic reticulum, lipid transporter activity, sulfur compound binding, steroid binding, glycosaminoglycan binding, alcohol binding, carboxylic ester hydrolase activity, lipid binding, receptor binding, coenzyme binding, adipogenesis, xenobiotic metabolism, fatty acid metabolism, coagulation, bile acid metabolism, peroxisome, or a combination thereof.

In some embodiments, the method further comprises administering to the subject in need thereof another agent that modulates expression and/or activity of one or more genes or gene products that have functions in HDAC3 targets, photodynamic therapy stress, CEBP targets, tolerant macrophage, response to salirasib, adult tissue stem module, klfl targets, anatomical structure formation involved in morphogenesis, circulatory system process, cellular response to external stimulus, response to wounding, cellular response to extracellular stimulus, cell activation, cellular response to oxygen containing compound, vesicle mediated transport, enzyme linked receptor protein signaling pathway, response to bacterium, regulation of catabolic process, response to ketone, regulation of cell adhesion, response to hormone, blood vessel morphogenesis, response to estrogen, response to radiation, response to extracellular stimulus, cellular response to nitrogen compound, regulation of catalytic activity, vasculature development, response to abiotic stimulus, response to drug, response to growth factor, regulation of protein metabolic process, transmembrane receptor protein tyrosine kinase signaling pathway, cellular response to peptide, hexose metabolic process, cellular response to stress, endocytosis, circulatory system development, response to starvation, hemostasis, response to molecule of bacterial origin, cell surface, peptidase regulator activity, molecular function regulator, peptidase inhibitor activity, phospholipid binding, TNF-a signaling via NFkB, or a combination thereof.

In some embodiments, the agent modulates expression and/or activity of one or more genes or gene products in Wnt pathway. In some embodiments, the agent modulates expression and/or activity of one or more genes or gene products that are markers of hepatic stem cells. In some embodiments, the expression and/or activity of the one or more genes or gene products is altered both in response to a zone-dependent injury and a zone-independent injury.

In some embodiments, the one or more genes or gene products comprises Gclc, Txnrd1, Lars2, Cyp4a14, Apoc2, Apoc1, Cyp2c29, Mt1, Mt2, Saa1, Saa2, Fgl1, Mup17, Mup18, Mup11, Gm23935, mmu-mir-6236, Ly6e, Rnase4, Saa4, Fgl1, Hp, Hpx, Lcn2, Orm1, Apcs, Orm2, Saa1, Saa2, Saa3, Sds, Tacc2, Igfbp1, Cxc11, Thrsp, Serpina3n, Lpin1, Steap4, Mt1, Mt2, Aldh3a2, Cyp2c37, Cyp2c29, Cyp8b1, Ces1d, Apoc1, Hsd17b13, Atp5h, Apoc2, Retsat, Mat1a, Angptl3, Chchd10, Hmgcs2, Cyp4a10, Cyp4a14, Gm26917, Lars2, Hyou1, Arrdc3, Mup12, Gm15564, Pdia3, Gm26924, Sephs2, Grip2, Krt8, Krt18, Plin2, Chka, Gclc, Srxn1, Hmox1, S100a8, S100a9, Mup15, Mup4, Ankrd55, Mup11, Mup5, Mup18, Mup9, Mup6, Mup17, Mup19, Alb, Pck1, Slc2a2, F2, Cyp2e1, Glu1, Arg1, Cdh1, Gls2, Ppargc1a, Sox9, Tbx3, Lgr5, Axin2, or a combination thereof.

In some embodiments, the one or more genes or gene products are selected from the genes or gene products in any one of Tables 1-8 or in all of Tables 1-8. In some embodiments, the agent induces regeneration and/or functional compensation of the organ or tissue. In some embodiments, the agent induces generation of cells that compensate function loss caused by the injury in the organ or tissue. In some embodiments, the agent induces cell proliferation in the organ or tissue. In some embodiments, the organ or tissue is liver, spleen, intestine, colon, bone marrow, an immune tissue or organ, or a tissue or organ of the gastrointestinal tract.

In some embodiments, the injury is an acute injury. In some embodiments, the injury is a chronic injury. In some embodiments, the injury is caused by a metabolic or toxic insult. In some embodiments, the injury is caused by high fat diet. In some embodiments, the injury is caused by a disease. In some embodiments, the injury is caused by a chronic disease. In some embodiments, the disease is a liver disease. In some embodiments, the liver disease is non-alcoholic fatty liver disease, non-alcoholic steatohepatitis, or cirrhosis. In some embodiments, the injury is a zone-independent injury. In some embodiments, the injury is a zone-dependent injury.

In another aspect, the present disclosure provides for a method of treating an injury in an organ or tissue, comprising determining expression of one or more genes from single cells in the organ or tissue at a first time point and a second time point; selecting a first subset of genes from the one or more genes, wherein expression of the first subset of genes at the first and the second time points are different; determining spatial locations of cells expressing the first subset of genes in the organ or tissue at the first and the second time points by an in situ hybridization assay; selecting a second subset of genes based on the spatial locations of the cells expressing the second subset of genes; and administering an agent that modulates expression and/or activity of one or more of the second subset of genes to a subject in need thereof.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized and the accompanying drawings of which:

FIG. 1A is an overview of an exemplary approach utilized for analysis. Briefly, massively-parallel single-cell RNA-sequencing (scRNA-seq) was performed on thousands of hepatocytes, before, during and after the proliferative phase, to assess changes in the transcriptional profile of the liver following zone-dependent (APAP) compared to zone-independent (PH) injury. To add spatial context and validation of the scRNA-seq, results were coupled with single molecule fluorescence in situ hybridization (smFISH) to measure and quantify the mRNA content of hepatocytes within the mouse liver. FIG. 1B includes the time course used in the analysis to assess the transcriptional environment during injury, recovery, and termination phases of liver regeneration.

FIG. 2A dimensional reduction technique, t-Stochastic Neighbour Embedding (t-SNE), to dataset reveals a diverse population of cells. Dataset subset of hepatocytes only for further analysis, which revealed distinct separation by condition. Each UT animal is distinct; injury samples cluster together by time point with 2-3 mice per condition. Clustering by SNN outlined in black. FIG. 2B Variation in pericentral hepatocyte (PCH) and periportal hepatocyte (PPH) signature, utilizing module scores for pericentral hepatocyte (PCH) and periportal hepatocyte (PPH) gene lists over the full dataset; there is clear pericentral to periportal gradients across all clusters except 6 hours post-APAP, due to the pericentral-specific injury in this model. PC1 captures technical variation (nGene, nUMI), PC2 partly captures PCH-PPH variation, creation of module score using PCH genes. Loss of PCH in APAP6 hr (A6) of FIG. 2B due to APAP toxicity. FIG. 2C heat map of untreated versus differential expression at each timepoint following injury. FIG. 2D Venn diagram of genes up-regulated following injury and down-regulated following injury in APAP and PH.

FIG. 3A representative plot of liver lobule for genes Cyp2e1 and Glu1 at different time points and for untreated cells. FIG. 3B tSNE of PCH2 and PPH2 with APAP 6 hour clusters circled. FIG. 3C Cyp2e1 and Glu1 expression smFISH shows extension of Cyp2e1 and Glu1 expression further into midlayer than WT. smFISH analysis confirms the loss of the Cyp2e1-positive cell population directly surrounding the central vein in the APAP model at 6 and 24 hrs following exposure. combinatorial analysis of smFISH using an algorithm to define cellular outlines (CellProfiler) and counting transcripts (FISH-quant) to analyze the large number of genes that span many liver functions. FISH-quant to convert spot counts from every cell outline defined by CellProfiler into a representative heat map of the liver lobule for a given gene. Interestingly, the Cyp2e1-positive area spans an increased number of cells at 24 and 48 hrs post-APAP. Functional compensation for loss of PCH due to APAP toxicity. Compensation in Cyp2e1 and Glu1 in midlayer in APAP and PH, with functional compensation due to loss of total liver mass in PH an even greater magnitude of compensation due to later loss of tissue in PH than APAP. Evaluation includes the number of transcripts/area from a defined reference point, (central vein).

FIG. 4—functional compensatory response can be seen in heat map and combinatorial analysis as described in FIG. 3A for other classic hepatic marker genes, including thioredoxin (Txnrd1), Albumin, gluconeogenesis gene Pck1, and the coagulation factor F2, and Gclc, a rate limiting enzyme in the synthesis of the anti-oxidant glutathione.

FIG. 5—Pathway activation for APA 6 hour, APAP 24 hr, APAP 48/96 hr, PH 3 hr, PH48 hr and PH 120 hr·g the ability to proliferate or if these are mutually exclusive events.

FIG. 6A—Exploration of the scRNA-seq data set provides cell cycling data scores at indicated time points. FIG. 6B percent cycling cells per sample at different time points post-injury and for UT cells. FIG. 6C shows cycling cells express hepatocyte functional genes at a lower level, Left panel: hepatocyte score vs. cell cycle score, Right panel: PC-PP Score vs. Cell Cycle Score shows no strong PP=PC preference in cycling cells. FIG. 6D PCNA/smFISH staining indicates cells upregulating Glu1 are largely PCNA negative, with proliferating and compensating cells appearing to be distinct populations. FIG. 6E includes imaging showing Glutamate Synthetase (Glu1), Proliferating Cells (PCNA), and Composite image of UT cells and treated cells at APAP 24 hr, APAP 48 hr, PH 3 hr, and PH 48 hr.

FIG. 7A—plots of Cyp2f2, Cyp2e1, Alb and Hepatocyte Sig1 at APAP 24 hour and PH 48 hr. FIG. 7B heatmap PH 48 hours. FIG. 7C heatmap APAP at 24 hours.

FIG. 8A—Wnt violin plot of UT and times APAP6, APAP24, APAP48, APAP96, PHX3, PHX48, PHX120. FIG. 8B Wnt Signaling Pathway activation score at each times A6, A24, A48, A96, PHX3, PHX48, and PHX120. FIG. 8C overview of partial hepatectomy study in wild type mice, B-cat knockout, and Wt1s knockout mice, and RNA/area of each mouse for Alb and Mt1baseline and at 24 hours.

FIG. 9A—liver zonation across the lobule, with accomplishment of its many functions through division of labor. FIG. 9B—zone-dependent injury model by acetaminophen (APAP) acute toxicity in the liver. FIG. 9C—H&E, TUNEL and FISHCyp2e1 imaging of APAP zone-dependent injury model.

FIG. 10—heatmap of marker genes per cluster injury response genes include APAP metabolism, Redox, Liver function, Tissue damage. UT up-reg genes mainly Mup (pheromone-related). Key: Gclc=Glutathione synthesis rate limiting step; Txnrd1=redox; Lars2=protein syth; Cyp=Cytochrome P450; Apo=lipid metabolism; Mt=redox, ion scavenging for proliferation; Saa=response to inflammation/tissue injury; Fgl1=Fibrinogen/clotting factor; Mup=major urinary protein/pheromone-related.

FIG. 11A—Return of Pericentral Hepatocytes (PCHs) at APAP 24 hr. Peak proliferative window known to occur 30-36 hours, PCH gene expression returns at APAP 24 hr. See cycling cells at 24 in RNA data in FIG. 6A-6E, not until 48 hr in PCNA staining CDKN1A (Cyclin Dependent Kinase Inhibitor 1A) (aka P21) is up in pre-proliferative time points. FIG. 11B depicts approach smFISH to explore spatial distribution of PCH gene expression across liver lobule, Profile pericentral-specific genes: Cyp2e1—responsible for APAP toxicity, Glu1—Highly restricted to PC region, correlated with Cyp2e1.

FIG. 12—Heatmap shows similar functional compensation in APAP and PH with up-regulation of genes and expression beyond typical PC boundary in both injury models with some shared response genes evident in top markers for each condition. Key: Gclc=Glutathione synthesis rate limiting step; Txnrd1=redox; Lars2=protein syth; Cyp=Cytochrome P450; Apo=lipid metabolism; Mt=redox, ion scavenging for proliferation; Saa=response to inflammation/tissue injury; Fgl1=Fibrinogen/clotting factor; Mup=major urinary protein/pheromone-related.

FIG. 13—charts of shared and unique pathways between APAP and PH treatment. See also Tables 9-11.

FIG. 14—smFISH confirms compensation for APAP specific genes include Txnrd response to ROS, Gclc=upregulation of glutathione production, which is consumed in APAP metabolism, smFISH confirms up-regulation of selected genes unique to APAP, with upregulation observed across the lobule.

FIG. 15—smFISH confirms compensation for shared genes, including upregulation of Mt1, Alb, F2 and Pck1 observed across the lobule in both APAP and PH injury.

FIG. 16—PCNA/smFISH staining of Glu1 shows cells upregulating Glu1 are largely PCNA negative; proliferating cells appear to be distinct from compensating cells.

FIG. 17A depiction of extension of current studies to other organs and from acute injury to chronic injury. FIG. 17B high fat diets can lead to liver and intestinal inflammation and cancer; liver stages as a result of high fat diet include Non Alcoholic Fatty Liver disease (NALFD)=fat accumulation; NASH (non-alcoholic steatohepatitis)=steatohepatitis, fat+inflammation+scarring; Cirrhosis=scar tissue replacing liver cells. FIG. 17C—schematic of protocol for biological expansion of studies utilizing high fat diet to study multiple organs including liver and intestines with subsequent sorting of cells, peripheral blood, and spleen and bone marrow (pilot only).

FIG. 18A-18G Hepatocytes respond to toxic and surgical liver injuries. FIG. 18A Time course depicting analysis time points during liver injury recovery following APAP overdose or PH. FIG. 18B Murine liver sections (5 μm, n=3) show necrotic TUNEL-positive (top) and proliferative PCNA-positive (bottom) cells. FIG. 18C Bar graphs quantifying total TUNEL- and PCNA-positive area. Error bars are s.e.m., P<0.05 (*) and <0.0001(****) calculated using Welch's ANOVA. FIG. 18D t-SNE plot of all high-quality hepatocytes (Methods) in the scRNA-Seq data set. Cells are colored by injury mode and time point. SNN clusters outlined in black. FIG. 18E Heatmap of marker genes for all clusters outlined in FIG. 18D. FIG. 18F, 18G Pericentral Hepatocyte Signature Score (PCH Signature Score) (left). Violin plot of normalized expression of Cyp2e1 (middle) and Glu1 (right); percent positive calculated as percentage of total cells in each condition above average normalized genes expression (dashed line). Untreated (UT) and each post-treatment are plotted for FIG. 18F APAP and FIG. 18G PH.

FIG. 19A-19E Functional compensation of hepatocytes following acute liver injury. FIG. 19A Schematic for staining and image quantification. FIG. 19B, 19C Images of liver section showing pericentral markers Cyp2e1 and Glu1 for untreated and each APAP-treated (FIG. 19B) or PH-treated (FIG. 19C) time point (left column). Cell outlined and colored by number of Cyp2e1 transcripts (dark gray, low; light gray, high) for each condition (middle column). Cell outlined and colored by number of Glu1 transcripts (black, low; light gray, high) for each condition (right column). FIG. 19D, 19E Quantification of gene expression intensity (y-axis) across the lobule (x-axis) for Cyp2e1 and Glu1. 90% of area under the curve (AUC) for UT is to the left dashed line. Total AUC posted above each plot. FIG. 19D APAP treated and FIG. 19E PH-treated.

FIG. 20A-20E. Shared and unique gene expression responses in acute livery injury models. FIG. 20A Venn diagram showing genes significantly upregulated in response to APAP and/or PH treatment compared to untreated. FIG. 20B Venn diagram of genes downregulated.

FIG. 20C Pathways with significant overlaps with differentially expressed genes. Significant pathways unique to APAP response (left), unique to PH response (middle) and significant in both responses (right). FIG. 20D Expression of oxidative stress response genes (Txnrd1 and Gclc) significantly upregulated in APAP treatment response. smFISH quantification shown as bar plot.

FIG. 20E Expression of genes representing specific hepatic functions (Alb, Pck1, F2 and Ma) as violin plots with upregulation in both APAP and PH-response by smFISH. Error bars are s.e.m.

FIG. 21A-21G. Identification and characterization of proliferating hepatocytes. FIG. 21A Violin plot of cell cycle score across all samples. Cycling cells (CC, larger dots) are identified as having a cell cycle score two standard deviations above average (dashed line). Percentage of cycling cells in each condition listed below each violin. FIG. 21B Scatter plot of Hepatocyte Score versus Cell Cycle Score. Horizontal line represents average Hepatocyte Score calculated over all untreated cells. Vertical line represents two standard deviations above the average cell cycle score.

FIG. 21C Violin plots on Hepatocyte Score for all APAP 24 hr cycling cells (CC) and an equal number of non-cycling cells (NC) from APAP24 (top) and the same for PH48 CC and NC (bottom). FIG. 21D Heatmap of marker genes of CC and NC in APAP 24 hr (left) and PH 48 hr (right). FIG. 21E Violin plots of Alb and Slc2a2 in CC and NC. FIG. 21F Co-expression of liver function genes (Slc2a2 and Alb) and PCNA. FIG. 21G Quantification of RNA expression and PCNA intensity. Functional hepatic markers are selectively maintained in proliferating hepatocytes. Alb shows a maintenance of expression (total RNA counts) in proliferating hepatocytes (mean PCNA intensity) while Slc2a2 reveals a negative correlation. Mean PCNA intensity (IF) and total RNA counts (smFISH) are plotted for individually segmented cells from three lobular areas/condition (A24 and P48) with Loess regression (line). *, effect size by Cohen's d>0.2; **, d>0.5; ***, d>0.8.

FIG. 22A-22D. Contribution of Wnt signaling to functional compensation of hepatocytes. FIG. 22A Wnt target gene expression score over cycling cells (CC) and non-cycling cells (NC) from A24 and PH48. FIG. 22B hepatocytes grouped by treatment condition (UT, A6, and P3) FIG. 22C Wnt knockout mouse models. FIG. 22D Hepatocyte marker expression (Alb and Arg1) in untreated and PH 24 hr for wild type (WT), endothelial cell Wntless KO (EC-Wls), and macrophage Wntless KO (Mac-Wtls) by smFISH. FIG. 22E Average RNA expression of hepatocyte markers (Alb, Arg1, Cyp2e1, and Glu1) in untreated and PH 24 hr for WT, EC-Wls, and Mac-Wtls by smFISH. Error bars represent s.e.m., P<0.05 (*), <0.005 (**), <0.0005 (***), and <0.0001 (****).

FIG. 23A-23B. Model of hepatocyte response to acute liver injury. FIG. 23A Wnt secretion from the pericentral endothelium functions in the maintenance of the pericentral gene expression gradient in normal, quiescent liver. FIG. 23B Wnt secretion from macrophages aids in functional compensation of midzonal and periportal hepatocytes during the pre-proliferation phase of acute liver injury. FIG. 23C Wnt secretion is essential for both functional compensation and activation of the proliferative response during regeneration. Compensating hepatocytes contribute to a maintenance of hepatic function, whereas proliferating hepatocytes selectively down-regulate a subset of hepatic genes.

FIG. 24A-24E scRNA-Seq Data Processing. FIG. 24A log(nGene) and log(nUMI) for each treatment condition. FIG. 24B t-SNE colored by mouse of origin. FIG. 24C t-SNE colored by cluster. Clusters are numbered from most to fewest member cells and annotated by cell type. FIG. 24D Violin plots for marker gene expression and percent mitochondrial content (percent.mito) in each cluster. FIG. 24E Hepatocyte Signature Scores for cells in good quality hepatocyte clusters, grouped by treatment condition. Cells scoring less than 3 standard deviations below the mean (dashed line) were filtered out as non-hepatocytes. Remaining cells were included in the high-quality hepatocyte dataset for further analysis.

FIG. 25A-25H Hepatocyte dataset analysis. FIG. 25A Principle Components Analysis (PCA) of hepatocyte dataset, PC1, PC2. Cells (dots) colored by treatment condition. FIG. 25B Violin plot of PC1 and PC2 scores for each cell, grouped by treatment condition. FIG. 25C t-sne, shaded by mouse of origin. FIG. 25D t-SNE colored by SNN clustering assignment. FIG. 25E PCA (PC1, PC2), colored by lognUMI, lognGene, Periportal Hepatocyte (PPH) Signature, and Pericentral Hepatocyte (PCH) Signature. Dark gray, low; light gray, medium; medium gray, high.

FIG. 25F Violin plots of genes used to calculate PPH Sig and PCH Sig, grouped by treatment condition. FIG. 25G PPH Signature cs. PCH Signature FIG. 25H t-sne colored by lognGene, lognUMI, percent mitochondrial content (percent.mito) and Hepatocyte Signature Score. Dark gray, low; light gray, medium; medium gray, high.

FIG. 26A-26D Workflow for smFISH data analysis. Overview summarizing different steps to obtain spatial expression gradients from smFISH images. Additional details in the methods section. Cells were automatically segmented with CellProfiler (FIG. 26A) and individual mRNA molecules were detected with FISH-quant (FIG. 26B). FIG. 26C In each image, the central vein (C.V.) and portal vein (P.V.) were manually annotated as polygons in ImJoy. FIG. 26D The normalized expression gradients were calculated with an ImJoy plugin as follows: for each RNA the distance to the polygon of the C.V. is calculated (positive values for mRNAs outside the C.V., negative values for mRNAs inside the C.V.). These distances are renormalized with the closest distance between the polygon of the C.V. and the center of mass of the polygon defining the P.V. A normalized distance value of 0 thus corresponds to a position at the C.V., whereas a value of 1 corresponds to a position at the center of mass of the P.V. Renormalized distances are summarized in a histogram with a bin width of 0.1. Finally, this histogram is normalized to consider that not all distances are equally represented in the image, e.g., a proportionally larger region close to the C.V. is present in the image. For this, Applicant calculated the distance of all pixels in the image to the C.V. and summarized these measurements in a histogram as described for the RNA distance. Each bin of the RNA distance histogram is then divided by the corresponding bin of the latter histogram.

FIG. 27A-27D Average RNA expression of Cyp2e1 and Glu1 following acute liver injury. Average RNA expression quantified by smFISH of Cyp2e1 FIG. 27A and Glu1 FIG. 27B following injury induced by APAP or PH. FIG. 27C Imaging of liver section showing periportal marker Arg1 for untreated and each APAP-treated or PH-treated time point (left column). Cell outlined and colored by number of Arg1 transcripts (dark gray, low; light gray, high) for each condition. FIG. 27D Quantification of gene expression intensity across the lobule for Arg1.

FIG. 28A-28D Hepatocyte gene expression following acute liver injury using smFISH. Imaging of liver section (5 μm) showing spatiotemporal maps of the number of transcripts counted (dark gray, low; light gray, high) for hepatic genes corresponding to FIG. 28A secreted proteins, FIG. 28B metabolism, FIG. 28C ion homeostasis, and FIG. 28D glucose homeostasis. Quantification of gene expression intensity (y-axis) across the lobule (x-axis) for each gene can be found below each image set. Total AUC is posted above each plot.

FIG. 29A-29I—Cycling Cells Figure. FIG. 29A Barplot of percentage of hepatocytes from each treatment condition which were classified as cycling cells. FIG. 29B t-SNE of all hepatocytes classified as cycling cells (CC, orange) or non-cycling cells (NC, aqua). FIG. 29C Violin plot of hepatocyte signature score (module score calculated over a list of hepatocyte genes) grouped by treatment condition. FIG. 29D Scatter plot of Periportal-pericentral Score (positive more periportal, negative more pericentral) versus Cell Cycle Score. Horizontal line represents average Periportal-pericentral Score calculated over all untreated cells. Vertical line represents two standard deviations above the average cell cycle score. FIG. 29E Periportal-pericentral Score for cycling (CC) and non-cycling (NC) cells in A24 (top; p=2.7e-04; Cohen's d effect size=−0.85) and PH48 (lower; p=0.25; Cohen's d effect size=−0.17). FIG. 29F violin plot lognUMI and Wnt target genes FIG. 29G for cycling (CC) and non-cycling (NC) cells for A24 and PH48. FIG. 29H Violin plot of Cdkn1a expression by treatment condition. FIG. 29I Pathway analysis of differentially expressed genes between CC and NC in APAP 24 hr and PH48 hr.

FIG. 30 Expression of putative hepatic stem cell markers following acute liver injury using smFISH. Imaging of liver section (5 μm) showing spatiotemporal maps of the number of transcripts counted (dark gray, low; light gray, high) for putative hepatic stem cell markers Axing, Sox9, and Tbx3. Quantification of gene expression intensity (y-axis) across the lobule (x-axis) for each gene can be found below each image set. Total AUC is posted above each plot.

FIG. 31A-31D Expression of hepatic genes in β-catenin KO mice following PH Average RNA expression of hepatic genes Alb (FIG. 31A), Arg1 (FIG. 31B), Cyp2e1 (FIG. 31C), and Glu1 (FIG. 31D) in WT and β-catenin KO mice in control and 24 hrs after PH. Functional compensation of Alb and Glu1 appears to be dependent on β-catenin, whereas compensation of Arg1 and Cyp2e1 is independent.

FIG. 32A-32C Quantification of macrophages in Wntless KO mice. FIG. 32A Quantification of gene expression intensity across the lobule for Cyp2e1, Arg1, Glu1, and Alb for WT, EC-Wls KO, and Mac-Wls KO mice. Represented is control versus 24 hrs following PH. FIG. 32B IHC staining for macrophages (F4/80+) in WT, EC-Wls KO, and Mac-Wls KO mice in control and 24 hrs after PH. FIG. 32C Quantification of macrophages (F4/80+) in WT, EC-Wls KO, and Mac-Wls KO mice in control and 24 hrs after PH. Results represent four 40× fields per group. Error bars are indicative of s.e.m.

FIG. 33A-33E. Identification of cell types in full dataset. FIG. 33A t-SNE of all sequenced cells passing initial filter, colored by compartment of origin. FIG. 33B t-SNE of full dataset colored by diet condition, CD (gray) or HFD (dark gray). FIG. 33C t-SNEs colored by module score calculated over marker genes for expected cell types, and number of genes captured (nGene), and percent mitochondrial content (percent.mito). Dark gray, low; light gray, intermediate; gray, high. FIG. 33D t-SNE showing SNN clustering (numbered with 0 being the cluster with the most cells, to 29, the cluster with the fewest). Clusters are annotated with cell type and, for samples primarily from a particular sample, major sample type of origin. FIG. 33E Stacked barplot showing fractional abundance of cells from each mouse in each cluster. HF mice are shown in medium to dark grays, CD mice in light gray.

FIG. 34A-34D Analysis of gut-originating populations. FIG. 34A t-SNE over gut-originating samples only, shaded by gut location (colon, distal small intestine, proximal small intestinal) and diet (CD, HF). FIG. 34B t-SNE with SNN clustering, clusters numbered from most to fewest cells. Clusters are annotated with cell type and sample of origin. FIG. 34C PPAR signature score calculated for CD (light shade, left) and HF (dark shade, right) cells in each cluster. Effect size calculated by Cohen's d: d<0.2=n (negligible); 0.2<d<0.5=* (small); 0.5<d<0.8=** (medium), d>0.8=*** (large). FIG. 34D Fractional abundance of HF and CD cells for each type of immune cell in gut dataset.

FIG. 35A-35I Analysis of liver-originating populations. FIG. 35A t-SNE of liver-originating samples, colored by sample type and diet condition. FIG. 35B SNN clustering. Clusters annotated with cell type. Cells originating from control lighter colored; cells originating from HFD vibrant colored. FIG. 35C Stacked barplot of fractional abundance of cells from each mouse in each identified liver sample cluster. HFD gray; CD dark gray. FIG. 35D Iterative clustering over non-parenchymal liver cells (NPCs). SNN clustering and cell type annotation. FIG. 35E t-SNE of NPC liver cells colored by diet condition. FIG. 35F IPA functions and upstream regulators upregulated in HFD cs. CD Kupffers FIG. 35G Iterative clustering over hepatocytes. Shaded by mouse of origin. SNN clusters outlined in black. FIG. 35H PPAR activation signature score over hepatocyte clusters. FIG. 35I CEBPA activation signature score over hepatocyte clusters. Effect size calculated by Cohen's d.

FIG. 36A-36C Stem cell gene expression in hepatocytes. FIG. 36A Expression of liver stem cell genes module score in CD and HFD hepatocytes. Liver stem cells called as scoring two standard deviations above the average (dashed line). Percentage of stem cells in each sample listed below. FIG. 36B Violin plots of expression of selected genes from the stem cell module. FIG. 36C Biaxial plot of Axin2 vs Lgr5 expression in identified stem cells.

FIG. 37A-37C Hepatocyte-derived organoid growth. FIG. 37A Heptocytes seeded in matrigel at 0 days. Growth at 8 and 14 days, small, growing organoids circled in CD Day 8. FIG. 37B Organoids after 2 months in culture. FIG. 37C ATPase growth assay on organoids after 2 months in culture. ANOVA with corrections for multiple comparisons.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboraotry Manual, 2nd edition 2013 (E. A. Greenfield ed.); Animal Cell Culture (1987) (R.I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +1-5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example, by puncture, or other collecting or sampling procedures. In certain embodiments, a biological sample may contain cells, such as preferably live cells, cell-derived vesicles, cell debris and/or extracellular fluid, such as particularly from liver, spleen, intestine, colon, bone marrow, an immune tissue or organ, or a tissue or organ of the gastrointestinal tract.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed. The term does not denote a particular age or sex. Thus, adult and newborn subjects, as well as fetuses, whether male or female, are intended to be covered. The term subject is further intended to include transgenic non-human species.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

The present disclosure provides for methods of treating injury in an organ or tissue. In general, the methods include administering to a subject in need thereof an agent that induces regeneration and functional compensation of the organ or tissue. The work described herein provides a mechanism by which the liver maintains essential physiological functions prior to the onset of cellular reconstitution and characterizes macrophage-derived WNT signals required for this compensation.

### Methods of Treatment

In one aspect, the present disclosure provides methods for treating an injury in an organ or tissue. In general, the methods include modulating the expression and/or activity of one or more genes. Such genes may have functions in regeneration of the organ or tissue. For examples, these genes may be involved in cell proliferation in the organ or tissues. Alternatively or additionally, these genes may have functions in functional compensation for loss of function in the organ or tissue due to the injury. For example, the genes may be involved in generating new cells or reprogramming existing cells to compensate the loss of function. In some embodiments, the methods include administering one or more agents that modulate the expression and/or activity of these genes or one or more genes of a defined pathway. The one or more agents may be administered to cells in an organ or tissue. The cells may be liver, spleen, intestine, colon, bone marrow, or an organ of the gastrointestinal tract. In certain embodiments, the cells are liver cells. The methods of treatment are to compensate or induce cell proliferation in which a tissue or organ is injured. In an aspect, the injury is an acute injury or chronic injury. The injury can be a disease, such as a metabolic disease, or can be due to surgery or toxicity exposure.

As used herein, the terms “treat”, “treating” and “treatment” refer to the alleviation or measurable lessening of one or more symptoms or measurable markers of an injury, disease or disorder. Measurable lessening includes any statistically significant decline in a measurable marker or symptom. In some embodiments, treatment is prophylactic treatment.

The treatment method may include administering a therapeutically effective amount of agent. The term “therapeutically effective amount” refers to an amount effective, at dosages and for periods of time necessary, to achieve the desired therapeutic result, e.g., a diminishment or prevention of effects associated with various disease states or conditions. The term “therapeutically effective amount” refers to an amount of a target gene or gene product modulator effective to treat or prevent a disease or disorder in a mammal. A therapeutically effective amount of a target gene or gene product modulator can vary according to factors such as the disease state, age, sex, and weight of the subject, and the ability of the therapeutic compound to elicit a desired response in the subject. A therapeutically effective amount is also one in which any toxic or detrimental effects of the therapeutic agent are outweighed by the therapeutically beneficial effects. In some embodiments, a therapeutically effective amount is an “effective amount”, which as used herein refers to the amount of therapeutic agent of pharmaceutical composition to alleviate at least one or some of the symptoms of the disease or disorder. An “effective amount” for purposes herein is thus determined by such considerations as are known in the art and is the amount to achieve improvement including, but not limited to, improved survival rate or more rapid recovery, or improvement or elimination of at least one symptom and other indicator of an immune or autoimmune disease which are appropriate measures by those skilled in the art. It should be noted that a target gene or gene product modulator as disclosed herein can be administered as a pharmaceutically acceptable salt and can be administered alone or as an active ingredient in combination with pharmaceutically acceptable carriers, diluents, adjuvants and vehicles.

The treatment method may include administering a prophylactically effective amount of agent. The term “prophylactically effective amount” refers to an amount of a target gene or gene product modulator which is effective, at dosages and for periods of time necessary, to achieve the desired prophylactic result, e.g., the amount of a target gene or gene product modulator. Typically, since a prophylactic dose of a target gene or gene product modulator is administered to a subject prior to or at an earlier stage of a disease, in some embodiments, a prophylactically effective amount is less than the therapeutically effective amount. A prophylactically effective amount of a target gene or gene product modulator is also one in which any toxic or detrimental effects of the compound are outweighed by the beneficial effects.

As used herein, the terms “prevent”, “preventing” and “prevention” refer to the avoidance or delay in manifestation of one or more symptoms or measurable markers of a disease or disorder. A delay in the manifestation of a symptom or marker is a delay relative to the time at which such symptom or marker manifests in a control or untreated subject with a similar likelihood or susceptibility of developing the disease or disorder. The terms “prevent,” “preventing,” and “prevention” include not only the avoidance or prevention of a symptom or marker of the disease, but also a reduced severity or degree of any one of the symptoms or markers of the disease, relative to those symptoms or markers in a control or non-treated individual with a similar likelihood or susceptibility of developing the disease or disorder, or relative to symptoms or markers likely to arise based on historical or statistical measures of populations affected by the disease or disorder.

By “reduced severity” is meant at least a 10% reduction in the severity or degree of a symptom or measurable disease marker, relative to a control or reference, e.g., at least 15%, 20%, 30%, 40%, 50%, 60%, 70%, 80%, 90%, 95%, 99% or even 100% (i.e., no symptoms or measurable markers).

As used herein, the terms “administering” and “introducing” are used interchangeably herein and refer to the placement of the agents of metabolic regulators of the present invention into a subject by a method or route which results in at least partial localization of a target gene or gene product modulator at a desired site. The compounds of the present invention can be administered by any appropriate route which results in an effective treatment in the subject. In some embodiments, administering is not systemic administration.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “systemic administration”, “administered systemically”, “peripheral administration” and “administered peripherally” as used herein mean the administration of a modulator such that it enters the animal's system and, thus, is subject to metabolism and other like processes, for example, subcutaneous administration.

### Target Genes and Pathways for Treating Injuries

The expression and/or activity of one or more genes or signaling pathways may be modulated for treating injuries in organs or tissues. As used herein, the term “gene” refers to a nucleic acid comprising an open reading frame encoding a polypeptide, including both exon and (optionally) intron sequences. A “gene” refers to coding sequence of a gene product, as well as non-coding regions of the gene product, including 5′UTR and 3′UTR regions, introns and the promoter of the gene product. The coding region of a gene can be a nucleotide sequence coding for an amino acid sequence or a functional RNA, such as tRNA, rRNA, catalytic RNA, siRNA, miRNA and antisense RNA. A gene can also be an mRNA or cDNA corresponding to the coding regions (e.g. exons and miRNA) optionally comprising 5′- or 3′ untranslated sequences linked thereto. These definitions generally refer to a single-stranded molecule, but in specific embodiments will also encompass an additional strand that is partially, substantially or fully complementary to the single-stranded molecule. Thus, a nucleic acid may encompass a single-stranded molecule or a double-stranded molecule that comprises one or more complementary strand(s) or “complement(s)” of a particular sequence comprising a molecule. As used herein, a single-stranded nucleic acid may be denoted by the prefix “ss”, a double stranded nucleic acid by the prefix “ds”, and a triple stranded nucleic acid by the prefix “is”. The term “gene” may refer to the segment of DNA involved in producing a polypeptide chain, it includes regions preceding and following the coding region as well as intervening sequences (introns and non-translated sequences, e.g., 5′- and 3′-untranslated sequences and regulatory sequences) between individual coding segments (exons). A gene can also be an amplified nucleic acid molecule produced in vitro comprising all or a part of the coding region and/or 5′- or 3′-untranslated sequences linked thereto.

All gene name symbols refer to the gene as commonly known in the art. Gene symbols may be those referred to by the HUGO Gene Nomenclature Committee (HGNC). Any reference to the gene symbol is a reference made to the entire gene or variants of the gene. The HUGO Gene Nomenclature Committee is responsible for providing human gene naming guidelines and approving new, unique human gene names and symbols. All human gene names and symbols can be searched at genenames.org, the HGNC website, and the guidelines for their formation are available there (genenames.org/guidelines).

In some embodiments, the one or more genes may have functions in regulation of proteolysis, chemical homeostasis, secretion by cells, regulation of hydrolase activity, regulation of body fluid levels, homeostatic process, wound healing, glycerolipid metabolic process, response to external stimuli, response to oxygen containing compounds, response to lipid, neutral lipid metabolic process, negative regulation of hydrolase activity, ion homeostasis, response to biotic stimulus, exocytosis, platelet degranulation, response to alcohol, regulated exocytosis, negative regulation of peptidase activity, extracellular matrix, secretory granule, platelet alpha granule, secretory granule lumen, secretory vesicle, vesicle lumen, blood microparticle, intracellular vesicle, extracellular space, cytoplasmic vesicle part, protein lipid complex, enzyme inhibitor activity, enzyme regulator activity, response to hypoxia, apoptosis, complement components functions and activities, or a combination thereof. In some cases, the methods include further administering an additional agent to modulate one or more genes that have functions in the pathways described in this paragraph.

In some embodiments, the one or more genes may have functions in PPAR signaling pathway, complement and coagulation cascades, PPARa activated gene expression, biological oxidations, metabolism of lipids and lipoproteins, nasopharyngeal carcinoma, intestine probiotics, plasma cell vs plasmablast, liver cancer, liver specific genes, multiple myeloma, response to UVb radiation, heart atrium vs ventricle, aging kidney no blood, endocrine therapy resistance, liver cancer, breast cancer basal, foxa2 targets, stem cell, lung cancer kras, tlx targets, liver cancer subclass g123, liver cancer subclass proliferation, liver cancer stem cell, liver cancer recurrence, liver cancer subclass s3, hepatoblastoma, liver development, liver hnf1a targets, matrisome, liver cancer krt19, fatty acid catabolic process, ammonium ion metabolic process, protein activation cascade, regulation of wound healing, response to estradiol, response to acid chemical, sterol homeostasis, lipoprotein metabolic process, fatty acid beta oxidation, protein maturation, regulation of locomotion, organic hydroxy compound metabolic process, organic acid biosynthetic process, monocarboxylic acid metabolic process, response to inorganic substance, regulation of vesicle mediated transport, regulation of fatty acid metabolic process, organic hydroxy compound transport, defense response, organophosphate ester transport, lipid homeostasis, secretion, anion transport, regulation of lipid biosynthetic process, response to xenobiotic stimulus, regulation of response to external stimulus, small molecule biosynthetic process, regulation of response to external stimulus, regulation of lipid metabolic process, amine metabolic process, autophagy, regulation of secretion, apoptotic signaling pathway, acute inflammatory response, regulation of catabolic process, maintenance of location, regulation of protein secretion, organic acid metabolic process, response to oxygen levels, regulation of cellular ketone metabolic process, organic acid catabolic process, regulation of response to wounding, regulation of extrinsic apoptotic signaling pathway, cellular lipid catabolic process, regulation of reactive oxygen species metabolic process, detoxification, regulation of peptidase activity, organic anion transport, inflammatory response, negative regulation of cell death, fatty acid metabolic process, lipid metabolic process, divalent inorganic cation homeostasis, regulation of endocytosis, alcohol metabolic process, immune response, cellular lipid metabolic process, monocarboxylic acid transport, negative regulation of apoptotic signaling pathway, multicellular organismal homeostasis, organic hydroxy compound biosynthetic process, regulation of cell death, lipid catabolic process, regulation of lipid metabolic process, regulation of steroid metabolic process, regulation of inflammatory response, response to toxic substance, cellular chemical homeostasis, regulation of transport, regulation of lipid catabolic process, regulation of immune effector process, lipid localization, regulation of proteolysis, regulation of secretion, regulation of response to wounding, regulation of multicellular organismal process, cellular homeostasis, single organism catabolic process, response to oxidative stress, behavior, acute phase response, regulation of response to external stimulus, regulation of apoptotic signaling pathway, regulation of cell proliferation, response to reactive oxygen species, endocytic vesicle, endoplasmic reticulum part, endoplasmic reticulum lumen, endoplasmic reticulum, lipid transporter activity, sulfur compound binding, steroid binding, glycosaminoglycan binding, alcohol binding, carboxylic ester hydrolase activity, lipid binding, receptor binding, coenzyme binding, adipogenesis, xenobiotic metabolism, fatty acid metabolism, coagulation, bile acid metabolism, peroxisome, or a combination thereof. In some cases, the methods include further administering an additional agent to modulate one or more genes that have functions in the pathways described in this paragraph.

In some embodiments, the one or more genes may have functions in HDAC3 targets, photodynamic therapy stress, CEBP targets, tolerant macrophage, response to salirasib, adult tissue stem module, klfl targets, anatomical structure formation involved in morphogenesis, circulatory system process, cellular response to external stimulus, response to wounding, cellular response to extracellular stimulus, cell activation, cellular response to oxygen containing compound, vesicle mediated transport, enzyme linked receptor protein signaling pathway, response to bacterium, regulation of catabolic process, response to ketone, regulation of cell adhesion, response to hormone, blood vessel morphogenesis, response to estrogen, response to radiation, response to extracellular stimulus, cellular response to nitrogen compound, regulation of catalytic activity, vasculature development, response to abiotic stimulus, response to drug, response to growth factor, regulation of protein metabolic process, transmembrane receptor protein tyrosine kinase signaling pathway, cellular response to peptide, hexose metabolic process, cellular response to stress, endocytosis, circulatory system development, response to starvation, hemostasis, response to molecule of bacterial origin, cell surface, peptidase regulator activity, molecular function regulator, peptidase inhibitor activity, phospholipid binding, TNF-a signaling via NFkB, or a combination thereof. In some cases, the methods include further administering an additional agent to modulate one or more genes that have functions in the pathways described in this paragraph.

In some examples, the one or more genes may be Gclc, Txnrd1, Lars2, Cyp4a14, Apoc2, Apoc1, Cyp2c29, Mt1, Mt2, Saa1, Saa2, Fgl1, Mup17, Mup18, Mup11, Gm23935, mmu-mir-6236, Ly6e, Rnase4, Saa4, Fgl1, Hp, Hpx, Lcn2, Orm1, Apcs, Orm2, Saa1, Saa2, Saa3, Sds, Tacc2, Igfbp1, Cxc11, Thrsp, Serpina3n, Lpin1, Steap4, Mt1, Mt2, Aldh3a2, Cyp2c37, Cyp2c29, Cyp8b1, Ces1d, Apoc1, Hsd17b13, Atp5h, Apoc2, Retsat, Mat1a, Angptl3, Chchd10, Hmgcs2, Cyp4a10, Cyp4a14, Gm26917, Lars2, Hyou1, Arrdc3, Mup12, Gm15564, Pdia3, Gm26924, Sephs2, Grip2, Krt8, Krt18, Plin2, Chka, Gclc, Srxn1, Hmox1, S100a8, S100a9, Mup15, Mup4, Ankrd55, Mup11, Mup5, Mup18, Mup9, Mup6, Mup17, Mup19, Alb, Pck1, Slc2a2, F2, Cyp2e1, Glu1, Arg1, Cdh1, Gls2, Ppargc1a, Sox9, Tbx3, Lgr5, Axin2, or a combination thereof.

In some examples, the one or more genes are selected from the genes listed in Tables 1 to 8.

The methods herein include modulating (e.g., using modulating agent(s)) one or more genes, e.g., at least 1, at least 2, at least 3, at least 4, at least 5, at least 6, at least 7, at least 8, at least 9, at least 10, at least 15, at least 20, at least 30, at least 40, at least 50, at least 60, at least 70, at least 80, at least 90, at least 100, at least 200, at least 300, at least 400, or at least 500 genes.

In some cases, the one or more genes may be in the Wnt pathway, a highly conserved signaling pathway also referred to as the Wnt/β-catenin pathway. Examples of such genes may be β-catenin, tumor suppressor gene product adenomatous polyposis coli (APC), axin, glycogen synthase kinase (GSK)-3β, TCF/LEF transcription factors (e.g. TCF4), crescent, groucho, CBP, frizzled receptor, frizzled related proteins, LRP, LRP5, LRP6, kremin, Dvl/Dsh (disheveled), dickkopf, GSK-3 binding protein (GBP), FRAT/GBP, Ebi, β-TrCP, Pin1, ICAT, E-cadherin, CKI, Lgs/BCL9, and Pygo, SFRP1, PP2A, ARFGAP1. Modulating agents and ligands of the pathway and gene products in the pathway are of particular interest. See, e.g., Shin et al., EBioMedicine 25 (2017) 22-31, incorporated by reference in its entirety.

In embodiments, the targets are Wnt that are expressed by macrophages. The macrophages may be distinct for their local environment or tissue type. Malsin et al., Fron. Immunol., 31 Jul. 2019, DOI: 10.3389/fimmu.2019.01813, incorporated herein by reference. In Malsin, et al., Table 1 identifies Wnt ligand study, role in macrophage biology, specific Wnt ligand as well as canonical or non-canonical pathway implicated, Table 1 incorporated herein specifically by reference. Wnt signaling in macrophages has included Wnt3a and Wnt5a in mycobacteria induced inflammatory responses. See, e.g. Schaale et al., DOI:10.1016/j.ejcb.2010.11.004; see also, Yang et al, Cell Death and Disease 9:793 (2018); Feng et al., JASN January 2018, 29 (1) 182-193; DOI:10.1681/ASN.2017040391, both incorporated herein by reference. In an aspect, the methods of treatment may comprise delivering a vector that can target liver macrophages specifically. In an aspect the vector can be configured to target hepatocytes such that the agent that stimulates macrophage Wnt signaling, one or more proteins or ligands of the Wnt pathway, such that the agent is released at the site of injury.

Methods of decreasing cancer susceptibility and/or inflammation are provided comprise administering a subject in need thereof an inhibitor of peroxisome proliferator-activated receptors (PPARs), alpha, gamma and delta (beta). PPARs are ligand-activated transcription factors of the nuclear hormone receptor superfamily, studied for how ligands and receptors modulate gene expression. See, e.g., Guan, The Italian Journal of Urology and Nephrology, 31 May 2002, 54(2):65-79; Rigano et al., Acta Pharm Sin B. 2017 July; 7(4): 427-438; doi: 10.1016/j.apsb.2017.05.005; Cheng et al., Mini Rev Med Chem. 2005 August; 5(8):741-53. See also, Liss et al, Biochimie. 2017 May; 136: 65-74. Doi: 10.1016/j.biochi.2016.11.009 and Souza mello, World J. Hepatol, doi: 10.4254/wjh.v7.i8.1012, both incorporated herein by reference, for discussion of PPAR receptors as targets in in Nonalcoholic fatty liver disease; and Peyrou et al, doi:10.1155/2012/757802 in liver disease and cancer, incorporated herein by reference.

A method of reducing risk of proliferation disorders or cancer in the liver comprising administering to a subject in need thereof aa modulating agent that increases expression of Sox9. Treatment with PGD2 has been shown to upregulate expression of endogenous Sox9, RAR agonists and CHX stimulation is also contemplated. See, Passeron et al, J Clin Invest. 2009 Apr. 1; 119(4): 954-963, published online 2009 Mar. 9. doi: 10.1172/JCI34015. Methods of reducing risk of proliferation disorders or cancer in the liver may also comprise administering an agent that decreases expression of Lrg5, (e.g. RNAi-mediated inhibition or other approaches, Br J Cancer. 2018 May 29; 118(11): 1410-1418, doi: 10.1038/s41416-018-0118-6, incorporated by reference) and Axin 2 (See, Gustafson et al., Front Pharmacol. 2017; 8: 285, doi: 10.3389/fphar.2017.00285; Bernkopf, D. B., Bruckner, M., Hadjihannas, M. V. et al. An aggregon in conductin/axin2 regulates Wnt/β-catenin signaling and holds potential for cancer therapy. Nat Commun 10, 4251 (2019). Doi:10.1038/s41467-019-12203-8, incorporated herein by reference). Methods of treatment increasing Sox9 and decreasing one or more of Lrg5 and Axin2 may also be utilized.

### Modulating Agents

In some embodiments, the methods herein include administering one or more agents that modulate the expression and/or activity of gene(s) and/or pathway.

For example, the methods may include administering at least 1, at least 2, at least 3, at least 4, at least 5, at least 6, at least 7, at least 8, at least 9, at least 10, at least 15, at least 20, at least 30, at least 40, at least 50, at least 60, at least 70, at least 80, at least 90, at least 100 modulating agents.

As will be clear to the skilled person, “modulating” can also involve affecting a change (which can either be an increase or a decrease) in affinity, avidity, specificity and/or selectivity of a target or antigen, for one or more of its targets compared to the same conditions but without the presence of a modulating agent. Again, this can be determined in any suitable manner and/or using any suitable assay known per se, depending on the target. In particular, an action as an inhibitor/antagonist or activator/agonist can be such that an intended biological or physiological activity is increased or decreased, respectively, by at least 5%, at least 10%, at least 25%, at least 50%, at least 60%, at least 70%, at least 80%, or 90% or more, compared to the biological or physiological activity in the same assay under the same conditions but without the presence of the inhibitor/antagonist agent or activator/agonist agent. Modulating can also involve activating the target or antigen or the mechanism or pathway in which it is involved.

“Altered expression” as intended herein may encompass modulating the activity of one or more endogenous gene products. Accordingly, “altered expression”, “altering expression”, “modulating expression”, or “detecting expression” or similar may be used interchangeably with, respectively, “altered expression or activity”, “altering expression or activity”, “modulating expression or activity”, or “detecting expression or activity” or similar. As used herein the term “altered expression” may particularly denote altered production of the recited gene products by a cell. As used herein, the term “gene product(s)” includes RNA transcribed from a gene (e.g., mRNA), or a polypeptide encoded by a gene or translated from RNA.

Modulation herein may include increasing, decreasing, abolishing, expression and/or activity of the one or more genes. The terms “increased” or “increase” or “upregulated” or “upregulate” as used herein generally mean an increase by a statically significant amount. For avoidance of doubt, “increased” means a statistically significant increase of at least 10% as compared to a reference level, including an increase of at least 20%, at least 30%, at least 40%, at least 50%, at least 60%, at least 70%, at least 80%, at least 90%, at least 100% or more, including, for example at least 2-fold, at least 3-fold, at least 4-fold, at least 5-fold, at least 10-fold increase or greater as compared to a reference level, as that term is defined herein.

The term “reduced” or “reduce” or “decrease” or “decreased” or “downregulate” or “downregulated” as used herein generally means a decrease by a statistically significant amount relative to a reference. For avoidance of doubt, “reduced” means statistically significant decrease of at least 10% as compared to a reference level, for example a decrease by at least 20%, at least 30%, at least 40%, at least 50%, or least 60%, or least 70%, or least 80%, at least 90% or more, up to and including a 100% decrease (i.e., absent level as compared to a reference sample), or any decrease between 10-100% as compared to a reference level, as that term is defined herein. The term “abolish” or “abolished” may in particular refer to a decrease by 100%, i.e., absent level as compared to a reference sample.

The term “agent” as used herein generally refers to any substance or composition, such as a chemical entity or biological product, or combination of chemical entities or biological products, capable of achieving a desired effect in a system, more particularly in a biological system, e.g., in a cell, tissue, organ, or an organism. In the present context, an agent may be exposed to, contacted with or introduced into an immune cell to modify at least one characteristic of the immune cell, such as to (inducibly) alter the expression or activity of the one or more genes or gene products as taught herein by the immune cell. Further in the present context, an agent may be administered to a subject to treat or prevent or control a disease or condition, for example by (inducibly) altering the expression or activity of the one or more genes or gene products as taught herein by immune cells of the subject.

In alternative embodiments, agents useful in the methods as disclosed herein are proteins and/or peptides or fragment thereof, which inhibit the gene expression of a target gene or gene product, or the function of a target protein. Such agents include, for example, but are not limited to protein variants, mutated proteins, therapeutic proteins, truncated proteins and protein fragments. Protein agents can also be selected from a group comprising mutated proteins, genetically engineered proteins, peptides, synthetic peptides, recombinant proteins, chimeric proteins, antibodies, midibodies, minibodies, triabodies, humanized proteins, humanized antibodies, chimeric antibodies, modified proteins and fragments thereof. As disclosed herein, a protein which inhibits the function of a target protein may be a soluble dominant negative form of the target protein or a functional fragment or variant thereof which inhibits wild-type full length target protein function.

In certain embodiments, the agents may be small molecules, antibodies, therapeutic antibody, antibody fragment, antibody-like protein scaffold, aptamer, protein, genetic modifying agent or small molecule. The chemical entity or biological product is preferably, but not necessarily a low molecular weight compound, but may also be a larger compound, or any organic or inorganic molecule effective in the given situation, including modified and unmodified nucleic acids such as antisense nucleic acids, RNAi, such as siRNA or shRNA, CRISPR-Cas systems, peptides, peptidomimetics, receptors, ligands, and antibodies, aptamers, polypeptides, nucleic acid analogues or variants thereof. Examples include an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof. Agents can be selected from a group comprising chemicals; small molecules; nucleic acid sequences; nucleic acid analogues; proteins; peptides; aptamers; antibodies; or fragments thereof. A nucleic acid sequence can be RNA or DNA, can be single or double stranded, and can be selected from a group comprising nucleic acid encoding a protein of interest, oligonucleotides, nucleic acid analogues, for example, peptide-nucleic acid (PNA), pseudo-complementary PNA (pc-PNA), locked nucleic acid (LNA), modified RNA (mod-RNA), single guide RNA etc. Such nucleic acid sequences include, for example, but are not limited to, nucleic acid sequence encoding proteins, for example, that act as transcriptional repressors, antisense molecules, ribozymes, small inhibitory nucleic acid sequences, for example, but are not limited to RNAi, shRNAi, siRNA, micro RNAi (mRNAi), antisense oligonucleotides, CRISPR guide RNA, for example, that target a CRISPR enzyme to a specific DNA target sequence, etc. A protein and/or peptide or fragment thereof can be any protein of interest, for example, but are not limited to mutated proteins; therapeutic proteins and truncated proteins, wherein the protein is normally absent or expressed at lower levels in the cell. Proteins can also be selected from a group comprising mutated proteins, genetically engineered proteins, peptides, synthetic peptides, recombinant proteins, chimeric proteins, antibodies, minibodies, humanized proteins, humanized antibodies, chimeric antibodies, modified proteins and fragments thereof. Alternatively, the agent can be intracellular within the cell as a result of introduction of a nucleic acid sequence into the cell and its transcription resulting in the production of the nucleic acid and/or protein modulator of a gene within the cell. In some embodiments, the agent is any chemical, entity or moiety, including without limitation synthetic and naturally-occurring non-proteinaceous entities. In certain embodiments the agent is a small molecule having a chemical moiety. Agents can be known to have a desired activity and/or property, or can be selected from a library of diverse compounds.

In some embodiments, the one or more agents may be small molecules. The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da.

In certain embodiments, the modulating agent can refer to a protein-binding agent that permits modulation or activity of proteins or disrupts interactions of proteins and other biomolecules, such as, but not limited to, disrupting protein-protein interaction, ligand-receptor interaction, or protein-nucleic acid interaction. Agents can also refer to DNA targeting or RNA targeting agents. Agents may include a fragment, derivative and analog of an active agent. The terms “fragment,” “derivative” and “analog” when referring to polypeptides as used herein refers to polypeptides which either retain substantially the same biological function or activity as such polypeptides. An analog includes a proprotein which can be activated by cleavage of the proprotein portion to produce an active mature polypeptide. Such agents include, but are not limited to, antibodies (“antibodies” includes antigen-binding portions of antibodies such as epitope- or antigen-binding peptides, paratopes, functional CDRs; recombinant antibodies; chimeric antibodies; humanized antibodies; nanobodies; tribodies; midibodies; or antigen-binding derivatives, analogs, variants, portions, or fragments thereof), protein-binding agents, nucleic acid molecules, small molecules, recombinant protein, peptides, aptamers, avimers and protein-binding derivatives, portions or fragments thereof.

As used herein, a “blocking” antibody or an antibody “antagonist” is one which inhibits or reduces biological activity of the antigen(s) it binds. For example, an antagonist antibody may bind a surface receptor or ligand and inhibit the ability of the receptor and ligand to induce an ILC class 2 inflammatory response. In certain embodiments, the blocking antibodies or antagonist antibodies or portions thereof described herein completely inhibit the biological activity of the antigen(s).

Antibodies may act as agonists or antagonists of the recognized polypeptides. For example, the present invention includes antibodies which disrupt receptor/ligand interactions either partially or fully. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III (Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996).

The antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques including, but not limited to specific chemical cleavage, acetylation, formylation, metabolic synthesis of tunicamycin, etc. Additionally, the derivative may contain one or more non-classical amino acids.

Methods for administering antibodies for therapeutic use is well known to one skilled in the art. In certain embodiments, small particle aerosols of antibodies or fragments thereof may be administered, preferably for treating a respiratory inflammatory disease (See e.g., Piazza et al., J. Infect. Dis., Vol. 166, pp. 1422-1424, 1992; and Brown, Aerosol Science and Technology, Vol. 24, pp. 45-56, 1996). In certain embodiments, antibodies are administered in metered-dose propellant driven aerosols. In preferred embodiments, antibodies are used as inhibitors or antagonists to depress inflammatory diseases or allergen-induced asthmatic responses. In certain embodiments, antibodies may be administered in liposomes, i.e., immunoliposomes (see, e.g., Maruyama et al., Biochim. Biophys. Acta, Vol. 1234, pp. 74-80, 1995). In certain embodiments, immunoconjugates, immunoliposomes or immunomicrospheres containing an agent of the present invention are administered by inhalation.

In some embodiments, the agents may be nucleic acid molecule. Exemplary nucleic acid molecules include aptamers, siRNA, artificial microRNA, interfering RNA or RNAi, dsRNA, ribozymes, antisense oligonucleotides, and DNA expression cassettes encoding said nucleic acid molecules. Preferably, the nucleic acid molecule is an antisense oligonucleotide. Antisense oligonucleotides (ASO) generally inhibit their target by binding target mRNA and sterically blocking expression by obstructing the ribosome. ASOs can also inhibit their target by binding target mRNA thus forming a DNA-RNA hybrid that can be a substance for RNase H. Preferred ASOs include Locked Nucleic Acid (LNA), Peptide Nucleic Acid (PNA), and morpholinos Preferably, the nucleic acid molecule is an RNAi molecule, i.e., RNA interference molecule. Preferred RNAi molecules include siRNA, shRNA, and artificial miRNA. The design and production of siRNA molecules is well known to one of skill in the art (e.g., Hajeri P B, Singh S K. Drug Discov Today. 2009 14(17-18):851-8). The nucleic acid molecule inhibitors may be chemically synthesized and provided directly to cells of interest. The nucleic acid compound may be provided to a cell as part of a gene delivery vehicle. Such a vehicle is preferably a liposome or a viral gene delivery vehicle.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection.

In certain embodiments, an agent may be a hormone, a cytokine, a lymphokine, a growth factor, a chemokine, a cell surface receptor ligand such as a cell surface receptor agonist or antagonist, or a mitogen.

Non-limiting examples of hormones include growth hormone (GH), adrenocorticotropic hormone (ACTH), dehydroepiandrosterone (DHEA), cortisol, epinephrine, thyroid hormone, estrogen, progesterone, testosterone, or combinations thereof.

Non-limiting examples of cytokines include lymphokines (e.g., interferon-γ, IL-2, IL-3, IL-4, IL-6, granulocyte-macrophage colony-stimulating factor (GM-CSF), interferon-γ, leukocyte migration inhibitory factors (T-LIF, B-LIF), lymphotoxin-alpha, macrophage-activating factor (MAF), macrophage migration-inhibitory factor (MIF), neuroleukin, immunologic suppressor factors, transfer factors, or combinations thereof), monokines (e.g., IL-1, TNF-alpha, interferon-α, interferon-β, colony stimulating factors, e.g., CSF2, CSF3, macrophage CSF or GM-CSF, or combinations thereof), chemokines (e.g., beta-thromboglobulin, C chemokines, CC chemokines, CXC chemokines, CX3C chemokines, macrophage inflammatory protein (MIP), or combinations thereof), interleukins (e.g., IL-1, IL-2, IL-3, IL-4, IL-5, IL-6, IL-7, IL-8, IL-9, IL-10, IL-11, IL-12, IL-13, IL-14, IL-15, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, or combinations thereof), and several related signaling molecules, such as tumour necrosis factor (TNF) and interferons (e.g., interferon-α, interferon-β, interferon-γ, interferon-λ, or combinations thereof).

Non-limiting examples of growth factors include those of fibroblast growth factor (FGF) family, bone morphogenic protein (BMP) family, platelet derived growth factor (PDGF) family, transforming growth factor beta (TGFbeta) family, nerve growth factor (NGF) family, epidermal growth factor (EGF) family, insulin related growth factor (IGF) family, hepatocyte growth factor (HGF) family, hematopoietic growth factors (HeGFs), platelet-derived endothelial cell growth factor (PD-ECGF), angiopoietin, vascular endothelial growth factor (VEGF) family, glucocorticoids, or combinations thereof.

Non-limiting examples of mitogens include phytohaemagglutinin (PHA), concanavalin A (conA), lipopolysaccharide (LPS), pokeweed mitogen (PWM), phorbol ester such as phorbol myristate acetate (PMA) with or without ionomycin, or combinations thereof.

Non-limiting examples of cell surface receptors the ligands of which may act as agents include Toll-like receptors (TLRs) (e.g., TLR1, TLR2, TLR3, TLR4, TLR5, TLR6, TLR7, TLR8, TLR9, TLR10, TLR11, TLR12 or TLR13), CD80, CD86, CD40, CCR7, or C-type lectin receptors.

### Genetic Modifying Agents

In certain embodiments, the one or more modulating agents may be a genetic modifying agent. The genetic modifying agent may comprise a CRISPR-Cas system, a zinc finger nuclease system, a TALEN, or a meganuclease.

### CRISPR-Cas System

In general, a CRISPR-Cas or CRISPR system as used in herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g, Shmakov et al. (2015) “Discovery and Functional Characterization of Diverse Class 2 CRISPR-Cas Systems”, Molecular Cell, DOI: dx.doi.org/10.1016/j.molce1.2015.10.008.

### Class 1 Systems

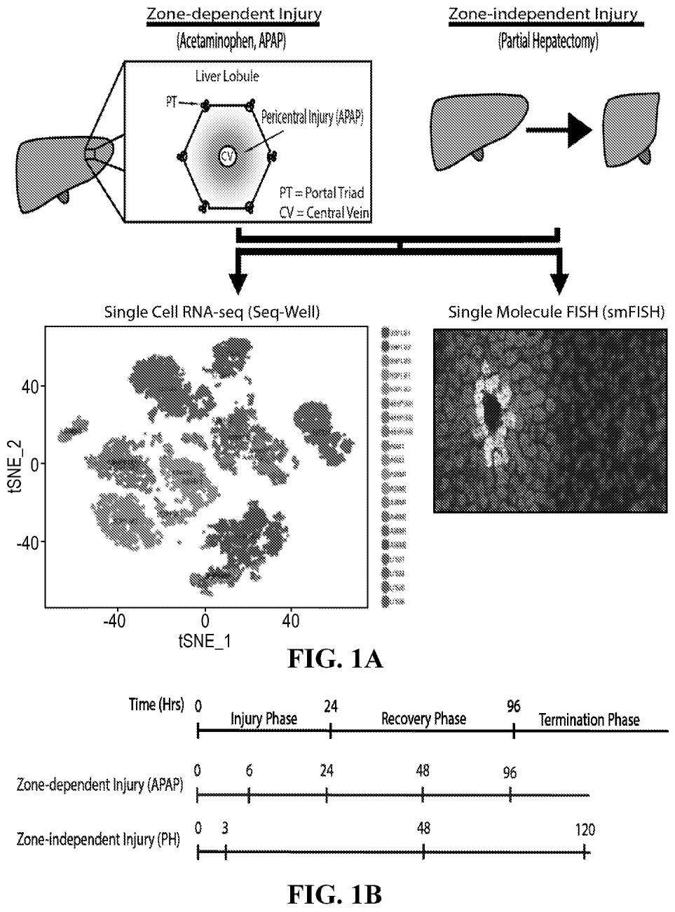

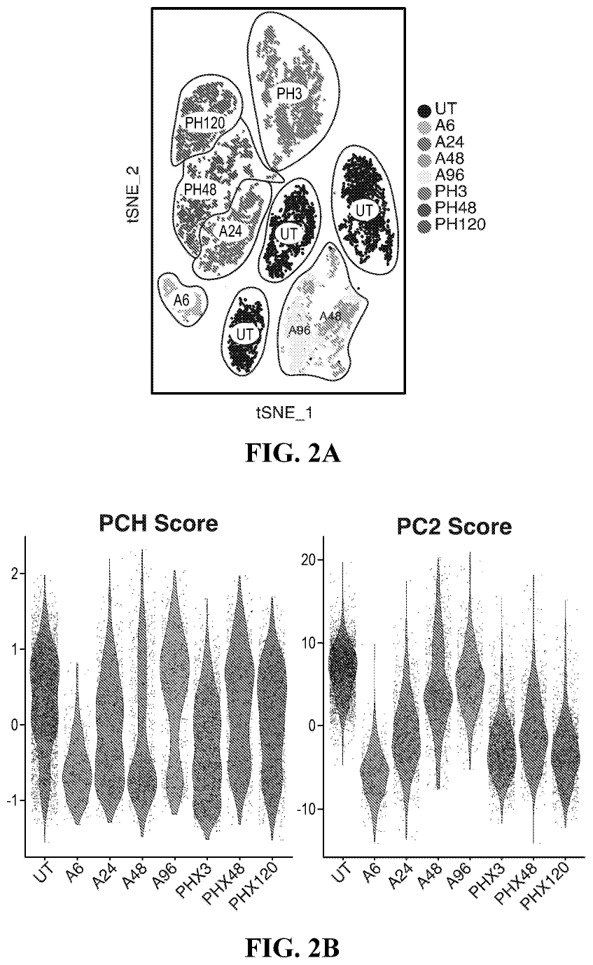

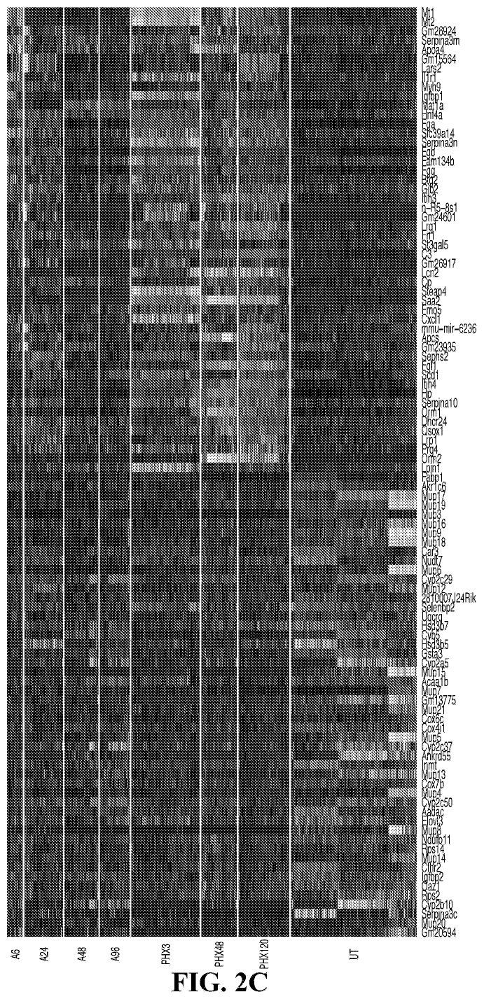

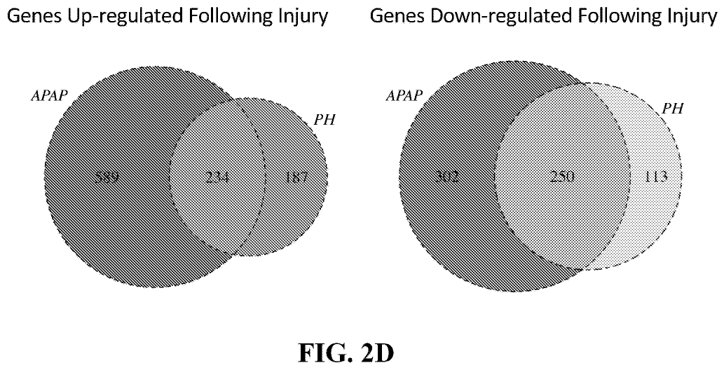

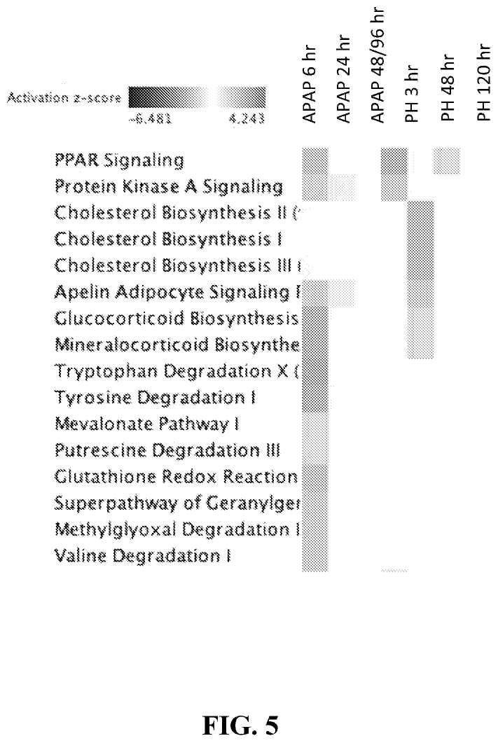

The methods, systems, and tools provided herein may be designed for use with Class 1 CRISPR proteins. In certain example embodiments, the Class 1 system may be Type I, Type III or Type IV Cas proteins as described in Makarova et al. “Evolutionary classification of CRISPR-Cas systems: a burst of class 2 and derived variants” Nature Reviews Microbiology, 18:67-81 (February 2020)., incorporated in its entirety herein by reference, and particularly as described in FIG. 1, p. 326. The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g. Cas1, Cas2, RNA nuclease), and/or one or more accessory proteins (e.g. Cas 4, DNA nuclease), CRISPR associated Rossman fold (CARF) domain containing proteins, and/or RNA transcriptase. Although Class 1 systems have limited sequence similarity, Class 1 system proteins can be identified by their similar architectures, including one or more Repeat Associated Mysterious Protein (RAMP) family subunits, e.g. Cas 5, Cas6, Cas7. RAMP proteins are characterized by having one or more RNA recognition motif domains. Large subunits (for example cas8 or cas10) and small subunits (for example, cas11) are also typical of Class 1 systems. See, e.g., FIGS. 1 and 2. Koonin E V, Makarova K S. 2019 Origins and evolution of CRISPR-Cas systems. Phil. Trans. R. Soc. B 374: 20180087, DOI: 10.1098/rstb.2018.0087. In one aspect, Class 1 systems are characterized by the signature protein Cas3. The Cascade in particular Class1 proteins can comprise a dedicated complex of multiple Cas proteins that binds pre-crRNA and recruits an additional Cas protein, for example Cas6 or Cas5, which is the nuclease directly responsible for processing pre-crRNA. In one aspect, the Type I CRISPR protein comprises an effector complex comprises one or more Cas5 subunits and two or more Cas7 subunits. Class 1 subtypes include Type I-A, I-B, I-C, I-U, I-D, I-E, and I-F, Type IV-A and IV-B, and Type III-A, III-C, and III-B. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al, the CRISPR Journal, v. 1, n5, FIG. 5.

### Class 2 Systems

In certain embodiments, the compositions, systems, and methods may be designed for use with Class 2 systems. In certain example embodiments, the Class 2 systems may be Type II, Type V, and Type VI systems as described in Makarova et al. “Evolutionary classification of CRISPR-Cas systems: a burst of class 2 and derived variants” Nature Reviews Microbiology, 18:67-81 (February 2020), incorporated herein by reference. The distinguishing feature of these types is that their effector complexes consist of a single, large, multi-domain protein. Type V systems differ from Type II effectors (e.g. Cas9) contain two nuclear domains that are each responsible for the cleavage of one strand of the target DNA, with the HNH nuclease inserted inside the Ruv-C like nuclease domain sequence. The Type V systems (e.g. Cas12) only contain a RuvC-like nuclease domain that cleaves both strands. Type VI (Cas13) are unrelated to the effectors of type II and V systems, contain two HEPN domains and target RNA. Cas13 proteins also display collateral activity that is triggered by target recognition. Some Type V systems have also been found to possess this collateral activity two single-stranded DNA in in vitro contexts.

In certain example embodiments, the CRISPR-Cas system comprises a Type II system. In certain example embodiments, the Type II system is a Cas9 system. In certain other example embodiments, the CRISPR-Cas systems is a Type V CRISPR-Cas systems. In certain example embodiments, the Type V CRISPR-Cas is Cas12a, Cas12b, or Cas12c.

### Guide Molecules

The modulating agents may comprise one or more guide molecules in CRISPR-Cas systems. As used herein, the term “guide sequence” and “guide molecule” in the context of a CRISPR-Cas system, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. The guide sequences made using the methods disclosed herein may be a full-length guide sequence, a truncated guide sequence, a full-length sgRNA sequence, a truncated sgRNA sequence, or an E+F sgRNA sequence. In some embodiments, the degree of complementarity of the guide sequence to a given target sequence, when optimally aligned using a suitable alignment algorithm, is about or more than about 50%, 60%, 75%, 80%, 85%, 90%, 95%, 97.5%, 99%, or more. In certain example embodiments, the guide molecule comprises a guide sequence that may be designed to have at least one mismatch with the target sequence, such that a RNA duplex formed between the guide sequence and the target sequence. Accordingly, the degree of complementarity is preferably less than 99%. For instance, where the guide sequence consists of 24 nucleotides, the degree of complementarity is more particularly about 96% or less. In particular embodiments, the guide sequence is designed to have a stretch of two or more adjacent mismatching nucleotides, such that the degree of complementarity over the entire guide sequence is further reduced. For instance, where the guide sequence consists of 24 nucleotides, the degree of complementarity is more particularly about 96% or less, more particularly, about 92% or less, more particularly about 88% or less, more particularly about 84% or less, more particularly about 80% or less, more particularly about 76% or less, more particularly about 72% or less, depending on whether the stretch of two or more mismatching nucleotides encompasses 2, 3, 4, 5, 6 or 7 nucleotides, etc. In some embodiments, aside from the stretch of one or more mismatching nucleotides, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence (or a sequence in the vicinity thereof) may be evaluated in a test tube by providing the target nucleic acid sequence, components of a nucleic acid-targeting complex, including the guide sequence to be tested and a control guide sequence different from the test guide sequence, and comparing binding or rate of cleavage at or in the vicinity of the target sequence between the test and control guide sequence reactions. Other assays are possible, and will occur to those skilled in the art. A guide sequence, and hence a nucleic acid-targeting guide RNA may be selected to target any target nucleic acid sequence.

In certain embodiments, the guide sequence or spacer length of the guide molecules is from 15 to 50 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer. In certain example embodiment, the guide sequence is 15, 16, 17, 18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nt.

In some embodiments, the guide sequence is an RNA sequence of between 10 to 50 nt in length, but more particularly of about 20 to 30 nt advantageously about 20 nt, 23 to 25 nt or 24 nt. The guide sequence is selected so as to ensure that it hybridizes to the target sequence. This is described more in detail below. Selection can encompass further steps which increase efficacy and specificity.

In some embodiments, the guide sequence has a canonical length (e.g., about 15 to 30 nt) is used to hybridize with the target RNA or DNA. In some embodiments, a guide molecule is longer than the canonical length (e.g., >30 nt) is used to hybridize with the target RNA or DNA, such that a region of the guide sequence hybridizes with a region of the RNA or DNA strand outside of the Cas-guide target complex. This can be of interest where additional modifications, such deamination of nucleotides, are of interest. In alternative embodiments, it is of interest to maintain the limitation of the canonical guide sequence length.

In some embodiments, the sequence of the guide molecule (direct repeat and/or spacer) is selected to reduce the degree of secondary structure within the guide molecule. In some embodiments, about or less than about 75%, 50%, 40%, 30%, 25%, 20%, 15%, 10%, 5%, 1%, or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example of folding algorithm is the online webserver RNAfold, developed at the Institute for Theoretical Chemistry at the University of Vienna, using the centroid structure prediction algorithm (see e.g., A. R. Gruber et al., 2008, Cell 106(1): 23-24; and P A Carr and G M Church, 2009, Nature Biotechnology 27(12): 1151-62).

In some embodiments, it is of interest to reduce the susceptibility of the guide molecule to RNA cleavage, such as to cleavage by Cas13. Accordingly, in particular embodiments, the guide molecule is adjusted to avoid cleavage by Cas13 or other RNA-cleaving enzymes.

In certain embodiments, the guide molecule comprises non-naturally occurring nucleic acids and/or non-naturally occurring nucleotides and/or nucleotide analogs, and/or chemical modifications. Preferably, these non-naturally occurring nucleic acids and non-naturally occurring nucleotides are located outside the guide sequence. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the invention, a guide nucleic acid comprises ribonucleotides and non-ribonucleotides. In one such embodiment, a guide comprises one or more ribonucleotides and one or more deoxyribonucleotides. In an embodiment of the invention, the guide comprises one or more non-naturally occurring nucleotide or nucleotide analog such as a nucleotide with phosphorothioate linkage, a locked nucleic acid (LNA) nucleotides comprising a methylene bridge between the 2′ and 4′ carbons of the ribose ring, or bridged nucleic acids (BNA). Other examples of modified nucleotides include 2′-O-methyl analogs, 2′-deoxy analogs, or 2′-fluoro analogs. Further examples of modified bases include, but are not limited to, 2-aminopurine, 5-bromo-uridine, pseudouridine, inosine, 7-methylguanosine. Examples of guide RNA chemical modifications include, without limitation, incorporation of 2′-O-methyl (M), 2′-O-methyl 3′phosphorothioate (MS), S-constrained ethyl(cEt), or 2′-O-methyl 3′thioPACE (MSP) at one or more terminal nucleotides. Such chemically modified guides can comprise increased stability and increased activity as compared to unmodified guides, though on-target vs. off-target specificity is not predictable. (See, Hendel, 2015, Nat Biotechnol. 33(9):985-9, doi: 10.1038/nbt.3290, published online 29 Jun. 2015 Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112:11870-11875; Sharma et al., Med Chem Comm., 2014, 5:1454-1471; Hendel et al., Nat. Biotechnol. (2015) 33(9): 985-989; Li et al., Nature Biomedical Engineering, 2017, 1, 0066 DOI:10.1038/s41551-017-0066). In some embodiments, the 5′ and/or 3′ end of a guide RNA is modified by a variety of functional moieties including fluorescent dyes, polyethylene glycol, cholesterol, proteins, or detection tags. (See Kelly et al., 2016, J. Biotech. 233:74-83). In certain embodiments, a guide comprises ribonucleotides in a region that binds to a target RNA and one or more deoxyribonucleotides and/or nucleotide analogs in a region that binds to Cas13. In an embodiment of the invention, deoxyribonucleotides and/or nucleotide analogs are incorporated in engineered guide structures, such as, without limitation, stem-loop regions, and the seed region. For Cas13 guide, in certain embodiments, the modification is not in the 5′-handle of the stem-loop regions. Chemical modification in the 5′-handle of the stem-loop region of a guide may abolish its function (see Li, et al., Nature Biomedical Engineering, 2017, 1:0066). In certain embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or 75 nucleotides of a guide is chemically modified. In some embodiments, 3-5 nucleotides at either the 3′ or the 5′ end of a guide is chemically modified. In some embodiments, only minor modifications are introduced in the seed region, such as 2′-F modifications. In some embodiments, 2′-F modification is introduced at the 3′ end of a guide. In certain embodiments, three to five nucleotides at the 5′ and/or the 3′ end of the guide are chemically modified with 2′-O-methyl (M), 2′-O-methyl 3′ phosphorothioate (MS), S-constrained ethyl(cEt), or 2′-O-methyl 3′ thioPACE (MSP). Such modification can enhance genome editing efficiency (see Hendel et al., Nat. Biotechnol. (2015) 33(9): 985-989). In certain embodiments, all of the phosphodiester bonds of a guide are substituted with phosphorothioates (PS) for enhancing levels of gene disruption. In certain embodiments, more than five nucleotides at the 5′ and/or the 3′ end of the guide are chemically modified with 2′-O-Me, 2′-F or S-constrained ethyl(cEt). Such chemically modified guide can mediate enhanced levels of gene disruption (see Ragdarm et al., 0215, PNAS, E7110-E7111). In an embodiment of the invention, a guide is modified to comprise a chemical moiety at its 3′ and/or 5′ end. Such moieties include, but are not limited to, amine, azide, alkyne, thio, dibenzocyclooctyne (DBCO), or Rhodamine. In certain embodiment, the chemical moiety is conjugated to the guide by a linker, such as an alkyl chain. In certain embodiments, the chemical moiety of the modified guide can be used to attach the guide to another molecule, such as DNA, RNA, protein, or nanoparticles. Such chemically modified guide can be used to identify or enrich cells generically edited by a CRISPR system (See Lee et al., eLife, 2017, 6:e25312, D01:10.7554).

In some embodiments, the modification to the guide is a chemical modification, an insertion, a deletion or a split. In some embodiments, the chemical modification includes, but is not limited to, incorporation of 2′-O-methyl (M) analogs, 2′-deoxy analogs, 2-thiouridine analogs, N6-methyladenosine analogs, 2′-fluoro analogs, 2-aminopurine, 5-bromo-uridine, pseudouridine (Ψ), N1-methylpseudouridine (me1Ψ), 5-methoxyuridine (5moU), inosine, 7-methylguanosine, 2′-O-methyl 3′phosphorothioate (MS), S-constrained ethyl(cEt), phosphorothioate (PS), or 2′-O-methyl 3′thioPACE (MSP). In some embodiments, the guide comprises one or more of phosphorothioate modifications. In certain embodiments, at least 1, 2, 3, 4, 5, 6, 7, 8, 9, 10, 11, 12, 13, 14, 15, 16, 17, 18, 19, 20, or 25 nucleotides of the guide are chemically modified. In certain embodiments, one or more nucleotides in the seed region are chemically modified. In certain embodiments, one or more nucleotides in the 3′-terminus are chemically modified. In certain embodiments, none of the nucleotides in the 5′-handle is chemically modified. In some embodiments, the chemical modification in the seed region is a minor modification, such as incorporation of a 2′-fluoro analog. In a specific embodiment, one nucleotide of the seed region is replaced with a 2′-fluoro analog. In some embodiments, 5 to 10 nucleotides in the 3′-terminus are chemically modified. Such chemical modifications at the 3′-terminus of the Cas13 CrRNA may improve Cas13 activity. In a specific embodiment, 1, 2, 3, 4, 5, 6, 7, 8, 9 or 10 nucleotides in the 3′-terminus are replaced with 2′-fluoro analogues. In a specific embodiment, 1, 2, 3, 4, 5, 6, 7, 8, 9 or 10 nucleotides in the 3′-terminus are replaced with 2′-O-methyl (M) analogs.

In some embodiments, the loop of the 5′-handle of the guide is modified. In some embodiments, the loop of the 5′-handle of the guide is modified to have a deletion, an insertion, a split, or chemical modifications. In certain embodiments, the modified loop comprises 3, 4, or 5 nucleotides. In certain embodiments, the loop comprises the sequence of UCUU, UUUU, UAUU, or UGUU.

In some embodiments, the guide molecule forms a stemloop with a separate non-covalently linked sequence, which can be DNA or RNA. In particular embodiments, the sequences forming the guide are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In some embodiments, these sequences can be functionalized to contain an appropriate functional group for ligation using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sufonyl, ally, propargyl, diene, alkyne, and azide. Once this sequence is functionalized, a covalent chemical bond or linkage can be formed between this sequence and the direct repeat sequence. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some embodiments, these stem-loop forming sequences can be chemically synthesized. In some embodiments, the chemical synthesis uses automated, solid-phase oligonucleotide synthesis machines with 2′-acetoxyethyl orthoester (2′-ACE) (Scaringe et al., J. Am. Chem. Soc. (1998) 120: 11820-11821; Scaringe, Methods Enzymol. (2000) 317: 3-18) or 2′-thionocarbamate (2′-TC) chemistry (Dellinger et al., J. Am. Chem. Soc. (2011) 133: 11540-11546; Hendel et al., Nat. Biotechnol. (2015) 33:985-989).

In certain embodiments, the guide molecule comprises (1) a guide sequence capable of hybridizing to a target locus and (2) a tracr mate or direct repeat sequence whereby the direct repeat sequence is located upstream (i.e., 5′) from the guide sequence. In a particular embodiment the seed sequence (i.e. the sequence essential critical for recognition and/or hybridization to the sequence at the target locus) of the guide sequence is approximately within the first 10 nucleotides of the guide sequence.

In a particular embodiment the guide molecule comprises a guide sequence linked to a direct repeat sequence, wherein the direct repeat sequence comprises one or more stem loops or optimized secondary structures. In particular embodiments, the direct repeat has a minimum length of 16 nts and a single stem loop. In further embodiments the direct repeat has a length longer than 16 nts, preferably more than 17 nts, and has more than one stem loop or optimized secondary structures. In particular embodiments the guide molecule comprises or consists of the guide sequence linked to all or part of the natural direct repeat sequence. A typical Type V or Type VI CRISPR-cas guide molecule comprises (in 3′ to 5′ direction or in 5′ to 3′ direction): a guide sequence a first complimentary stretch (the “repeat”), a loop (which is typically 4 or 5 nucleotides long), a second complimentary stretch (the “anti-repeat” being complimentary to the repeat), and a poly A (often poly U in RNA) tail (terminator). In certain embodiments, the direct repeat sequence retains its natural architecture and forms a single stem loop. In particular embodiments, certain aspects of the guide architecture can be modified, for example by addition, subtraction, or substitution of features, whereas certain other aspects of guide architecture are maintained. Preferred locations for engineered guide molecule modifications, including but not limited to insertions, deletions, and substitutions include guide termini and regions of the guide molecule that are exposed when complexed with the CRISPR-Cas protein and/or target, for example the stemloop of the direct repeat sequence.

In particular embodiments, the stem comprises at least about 4 bp comprising complementary X and Y sequences, although stems of more, e.g., 5, 6, 7, 8, 9, 10, 11 or 12 or fewer, e.g., 3, 2, base pairs are also contemplated. Thus, for example X2-10 and Y2-10 (wherein X and Y represent any complementary set of nucleotides) may be contemplated. In one aspect, the stem made of the X and Y nucleotides, together with the loop will form a complete hairpin in the overall secondary structure; and, this may be advantageous and the number of base pairs can be any amount that forms a complete hairpin. In one aspect, any complementary X:Y basepairing sequence (e.g., as to length) is tolerated, so long as the secondary structure of the entire guide molecule is preserved. In one aspect, the loop that connects the stem made of X:Y basepairs can be any sequence of the same length (e.g., 4 or 5 nucleotides) or longer that does not interrupt the overall secondary structure of the guide molecule. In one aspect, the stemloop can further comprise, e.g. an MS2 aptamer. In one aspect, the stem comprises about 5-7 bp comprising complementary X and Y sequences, although stems of more or fewer basepairs are also contemplated. In one aspect, non-Watson Crick basepairing is contemplated, where such pairing otherwise generally preserves the architecture of the stem loop at that position.

In particular embodiments the natural hairpin or stem loop structure of the guide molecule is extended or replaced by an extended stem loop. It has been demonstrated that extension of the stem can enhance the assembly of the guide molecule with the CRISPR-Cas protein (Chen et al. Cell. (2013); 155(7): 1479-1491). In particular embodiments the stem of the stemloop is extended by at least 1, 2, 3, 4, 5 or more complementary basepairs (i.e. corresponding to the addition of 2, 4, 6, 8, 10 or more nucleotides in the guide molecule). In particular embodiments these are located at the end of the stem, adjacent to the loop of the stemloop.

In particular embodiments, the susceptibility of the guide molecule to RNAses or to decreased expression can be reduced by slight modifications of the sequence of the guide molecule which do not affect its function. For instance, in particular embodiments, premature termination of transcription, such as premature transcription of U6 Pol-III, can be removed by modifying a putative Pol-III terminator (4 consecutive U's) in the guide molecules sequence. Where such sequence modification is required in the stemloop of the guide molecule, it is preferably ensured by a basepair flip.

In a particular embodiment, the direct repeat may be modified to comprise one or more protein-binding RNA aptamers. In a particular embodiment, one or more aptamers may be included such as part of optimized secondary structure. Such aptamers may be capable of binding a bacteriophage coat protein as detailed further herein.

In some embodiments, the guide molecule forms a duplex with a target RNA comprising at least one target cytosine residue to be edited. Upon hybridization of the guide RNA molecule to the target RNA, the cytidine deaminase binds to the single strand RNA in the duplex made accessible by the mismatch in the guide sequence and catalyzes deamination of one or more target cytosine residues comprised within the stretch of mismatching nucleotides.

A guide sequence, and hence a nucleic acid-targeting guide RNA may be selected to target any target nucleic acid sequence. The target sequence may be mRNA.

In certain embodiments, the target sequence should be associated with a PAM (protospacer adjacent motif) or PFS (protospacer flanking sequence or site); that is, a short sequence recognized by the CRISPR complex. Depending on the nature of the CRISPR-Cas protein, the target sequence should be selected such that its complementary sequence in the DNA duplex (also referred to herein as the non-target sequence) is upstream or downstream of the PAM. In the embodiments of the present invention where the CRISPR-Cas protein is a Cas13 protein, the complementary sequence of the target sequence is downstream or 3′ of the PAM or upstream or 5′ of the PAM. The precise sequence and length requirements for the PAM differ depending on the Cas13 protein used, but PAMs are typically 2-5 base pair sequences adjacent the protospacer (that is, the target sequence). Examples of the natural PAM sequences for different Cas13 orthologues are provided herein below and the skilled person will be able to identify further PAM sequences for use with a given Cas13 protein.

Further, engineering of the PAM Interacting (PI) domain may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously.

In particular embodiment, the guide is an escorted guide. By “escorted” is meant that the CRISPR-Cas system or complex or guide is delivered to a selected time or place within a cell, so that activity of the CRISPR-Cas system or complex or guide is spatially or temporally controlled. For example, the activity and destination of the 3 CRISPR-Cas system or complex or guide may be controlled by an escort RNA aptamer sequence that has binding affinity for an aptamer ligand, such as a cell surface protein or other localized cellular component. Alternatively, the escort aptamer may for example be responsive to an aptamer effector on or in the cell, such as a transient effector, such as an external energy source that is applied to the cell at a particular time.

The escorted CRISPR-Cas systems or complexes have a guide molecule with a functional structure designed to improve guide molecule structure, architecture, stability, genetic expression, or any combination thereof. Such a structure can include an aptamer.

Aptamers are biomolecules that can be designed or selected to bind tightly to other ligands, for example using a technique called systematic evolution of ligands by exponential enrichment (SELEX; Tuerk C, Gold L: “Systematic evolution of ligands by exponential enrichment: RNA ligands to bacteriophage T4 DNA polymerase.” Science 1990, 249:505-510). Nucleic acid aptamers can for example be selected from pools of random-sequence oligonucleotides, with high binding affinities and specificities for a wide range of biomedically relevant targets, suggesting a wide range of therapeutic utilities for aptamers (Keefe, Anthony D., Supriya Pai, and Andrew Ellington. “Aptamers as therapeutics.” Nature Reviews Drug Discovery 9.7 (2010): 537-550). These characteristics also suggest a wide range of uses for aptamers as drug delivery vehicles (Levy-Nissenbaum, Etgar, et al. “Nanotechnology and aptamers: applications in drug delivery.” Trends in Biotechnology 26.8 (2008): 442-449; and, Hicke B J, Stephens A W. “Escort aptamers: a delivery service for diagnosis and therapy.” J Clin Invest 2000, 106:923-928.). Aptamers may also be constructed that function as molecular switches, responding to a que by changing properties, such as RNA aptamers that bind fluorophores to mimic the activity of green fluorescent protein (Paige, Jeremy S., Karen Y. Wu, and Samie R. Jaffrey. “RNA mimics of green fluorescent protein.” Science 333.6042 (2011): 642-646). It has also been suggested that aptamers may be used as components of targeted siRNA therapeutic delivery systems, for example targeting cell surface proteins (Zhou, Jiehua, and John J. Rossi. “Aptamer-targeted cell-specific RNA interference.” Silence 1.1 (2010): 4).

Accordingly, in particular embodiments, the guide molecule is modified, e.g., by one or more aptamer(s) designed to improve guide molecule delivery, including delivery across the cellular membrane, to intracellular compartments, or into the nucleus. Such a structure can include, either in addition to the one or more aptamer(s) or without such one or more aptamer(s), moiety(ies) so as to render the guide molecule deliverable, inducible or responsive to a selected effector. The invention accordingly comprehends a guide molecule that responds to normal or pathological physiological conditions, including without limitation pH, hypoxia, O₂ concentration, temperature, protein concentration, enzymatic concentration, lipid structure, light exposure, mechanical disruption (e.g. ultrasound waves), magnetic fields, electric fields, or electromagnetic radiation. Inducible systems and energy application can be as described for example, in International Patent Publication WO2019232542 at [0275]-[0302], incorporated herein by reference.

In particular embodiments, the guide molecule is modified by a secondary structure to increase the specificity of the CRISPR-Cas system and the secondary structure can protect against exonuclease activity and allow for 5′ additions to the guide sequence also referred to herein as a protected guide molecule.

In one aspect, the invention provides for hybridizing a “protector RNA” to a sequence of the guide molecule, wherein the “protector RNA” is an RNA strand complementary to the 3′ end of the guide molecule to thereby generate a partially double-stranded guide RNA. In an embodiment of the invention, protecting mismatched bases (i.e. the bases of the guide molecule which do not form part of the guide sequence) with a perfectly complementary protector sequence decreases the likelihood of target RNA binding to the mismatched basepairs at the 3′ end. In particular embodiments of the invention, additional sequences comprising an extended length may also be present within the guide molecule such that the guide comprises a protector sequence within the guide molecule. This “protector sequence” ensures that the guide molecule comprises a “protected sequence” in addition to an “exposed sequence” (comprising the part of the guide sequence hybridizing to the target sequence). In particular embodiments, the guide molecule is modified by the presence of the protector guide to comprise a secondary structure such as a hairpin. Advantageously there are three or four to thirty or more, e.g., about 10 or more, contiguous base pairs having complementarity to the protected sequence, the guide sequence or both. It is advantageous that the protected portion does not impede thermodynamics of the CRISPR-Cas system interacting with its target. By providing such an extension including a partially double stranded guide molecule, the guide molecule is considered protected and results in improved specific binding of the CRISPR-Cas complex, while maintaining specific activity.

In particular embodiments, use is made of a truncated guide (tru-guide), i.e., a guide molecule which comprises a guide sequence which is truncated in length with respect to the canonical guide sequence length. As described by Nowak et al. (Nucleic Acids Res (2016) 44 (20): 9555-9564), such guides may allow catalytically active CRISPR-Cas enzyme to bind its target without cleaving the target RNA. In particular embodiments, a truncated guide is used which allows the binding of the target but retains only nickase activity of the CRISPR-Cas enzyme.

In addition to the above CRISPR-Cas systems, the CRISPR-Cas may be a base editor version, thereof i.e. a catalytically dead Cas linked or fused to a nucleotide deaminase domain. The Cas may be a RNA-binding (e.g. Type VI) on DNA-binding Cas (Type II or V). In certain embodiments, the compositions, systems, and methods may be designed for use with Class 2 systems. In certain example embodiments, the Class 2 systems may be Type II, Type V, and Type VI systems as described in Makarova et al. “Evolutionary classification of CRISPR-Cas systems: a burst of class 2 and derived variants” Nature Reviews Microbiology, 18:67-81 (February 2020), incorporated herein by reference. The distinguishing feature of these types is that their effector complexes consist of a single, large, multi-domain protein. Type V systems differ from Type II effectors (e.g. Cas9) contain two nuclear domains that are each responsible for the cleavage of one strand of the target DNA, with the HNH nuclease inserted inside the Ruv-C like nuclease domain sequence. The Type V systems (e.g. Cas12) only contain a RuvC-like nuclease domain that cleaves both strands. Type VI (Cas13) are unrelated to the effectors of type II and V systems, contain two HEPN domains and target RNA. Cas13 proteins also display collateral activity that is triggered by target recognition. Some Type V systems have also been found to possess this collateral activity two single-stranded DNA in in vitro contexts.

In certain example embodiments, the CRISPR-Cas system comprises a Type II system. In certain example embodiments, the Type II system is a Cas9 system. In certain other example embodiments, the CRISPR-Cas systems is a Type V CRISPR-Cas systems. In certain example embodiments, the Type V CRISPR-Cas is Cas12a, Cas12b, or Cas12c.

The present invention also contemplates use of the CRISPR-Cas system and the base editor described herein, for treatment in a variety of diseases and disorders. In some embodiments, the invention described herein relates to a method for therapy in which cells are edited ex vivo by CRISPR or the base editor to modulate at least one gene, with subsequent administration of the edited cells to a patient in need thereof. In some embodiments, the editing involves knocking in, knocking out or knocking down expression of at least one target gene in a cell. In particular embodiments, the editing inserts an exogenous, gene, minigene or sequence, which may comprise one or more exons and introns or natural or synthetic introns into the locus of a target gene, a hot-spot locus, a safe harbor locus of the gene genomic locations where new genes or genetic elements can be introduced without disrupting the expression or regulation of adjacent genes, or correction by insertions or deletions one or more mutations in DNA sequences that encode regulatory elements of a target gene. In some embodiment, the editing comprise introducing one or more point mutations in a nucleic acid (e.g., a genomic DNA) in a target cell.

The present disclosure also provides for a base editing system. In general, such a system may comprise a deaminase (e.g., an adenosine deaminase or cytidine deaminase) fused with a Cas protein. The Cas protein may be a dead Cas protein or a Cas nickase protein. In certain examples, the system comprises a mutated form of an adenosine deaminase fused with a dead CRISPR-Cas or CRISPR-Cas nickase. The mutated form of the adenosine deaminase may have both adenosine deaminase and cytidine deaminase activities.

In one aspect, the present disclosure provides an engineered adenosine deaminase. The engineered adenosine deaminase may comprise one or more mutations herein. In some embodiments, the engineered adenosine deaminase has cytidine deaminase activity. In certain examples, the engineered adenosine deaminase has both cytidine deaminase activity and adenosine deaminase. In some cases, the modifications by base editors herein may be used for targeting post-translational signaling or catalysis.

In one aspect, the invention provides a method of modifying or editing a target transcript in a eukaryotic cell. In some embodiments, the method comprises allowing a CRISPR-Cas effector module complex to bind to the target polynucleotide to effect RNA base editing, wherein the CRISPR-Cas effector module complex comprises a Cas effector module complexed with a guide sequence hybridized to a target sequence within said target polynucleotide, wherein said guide sequence is linked to a direct repeat sequence. In some embodiments, the Cas effector module comprises a catalytically inactive CRISPR-Cas protein. In some embodiments, the guide sequence is designed to introduce one or more mismatches to the RNA/RNA duplex formed between the target sequence and the guide sequence. In particular embodiments, the mismatch is an A-C mismatch. In some embodiments, the Cas effector may associate with one or more functional domains (e.g. via fusion protein or suitable linkers). In some embodiments, the effector domain comprises one or more cytidine or adenosine deaminases that mediate endogenous editing of via hydrolytic deamination. In particular embodiments, the effector domain comprises the adenosine deaminase acting on RNA (ADAR) family of enzymes. In particular embodiments, the adenosine deaminase protein or catalytic domain thereof is capable of deaminating adenosine or cytidine in RNA or is an RNA specific adenosine deaminase and/or is a bacterial, human, cephalopod, or Drosophila adenosine deaminase protein or catalytic domain thereof, preferably TadA, more preferably ADAR, optionally huADAR, optionally (hu)ADAR1 or (hu)ADAR2, preferably huADAR2 or catalytic domain thereof. See, e.g. Levy et al., doi:10.1038/s41551-019-0501-5, Rees et al, doi: 10.1038/s41467-019-09983-4; Komor et al, m Nature 533(7603), 420-424, Gaudellim et al, Nature 551 (7681), 464-471, Lee, et al., Nature Commun. 9:4804 1-5(2018), Song et al., Biomed End. 36, 536-539 (2018), Lee et al., Sci. Rep. 9, 1662 (2019), Thuronyi, et al., Nat. Biotechnol. 37, 1070-1079 (2019), Anzalone, et al., nature 576 149-157 (2019), and Richter et al., Nat Biotechnol in press (2020), all incorporated herein by reference. Reference is also made to International Patent Publication Nos. WO 2019/005884, WO 2019/005886, WO 2020/028555, WO 2019/060746, WO 2019/071048, WO 2019/084063, and Abudayyeh et al., Science 365:6451, 382-386, doi: 10.1126/science.aax7063, incorporated herein by reference.

### TALE Systems

The modulating agents may be one or more components of a TALE system, or nucleic acids encoding thereof. As disclosed herein editing can be made by way of the transcription activator-like effector nucleases (TALENs) system. Transcription activator-like effectors (TALEs) can be engineered to bind practically any desired DNA sequence. Exemplary methods of genome editing using the TALEN system can be found for example in Cermak T. Doyle E L. Christian M. Wang L. Zhang Y. Schmidt C, et al. Efficient design and assembly of custom TALEN and other TAL effector-based constructs for DNA targeting. Nucleic Acids Res. 2011; 39:e82; Zhang F. Cong L. Lodato S. Kosuri S. Church G M. Arlotta P Efficient construction of sequence-specific TAL effectors for modulating mammalian transcription. Nat Biotechnol. 2011; 29:149-153 and U.S. Pat. Nos. 8,450,471, 8,440,431 and 8,440,432, all of which are specifically incorporated by reference. Further description of TALE systems are as described in [0340]-[0351] of WO 2019232542, incorporated herein by reference.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention, fragments of the N-terminal and/or C-terminal capping regions can also be utilized with the TALE polypeptides.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4× domain or a Krüppel-associated box (KRAB) or fragments of the KRAB domain. In some embodiments the effector domain is an enhancer of transcription (i.e. an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination the activities described herein.

### Zn-Finger Nucleases

The one or more agents may comprise Zn-finger nucleases or nucleic acids encoding thereof. Other preferred tools for genome editing for use in the context of this invention include zinc finger systems and TALE systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### Meganucleases

As disclosed herein editing can be made by way of meganucleases, which are endodeoxyribonucleases characterized by a large recognition site (double-stranded DNA sequences of 12 to 40 base pairs). Exemplary method for using meganucleases can be found in U.S. Pat. Nos. 8,163,514, 8,133,697, 8,021,867, 8,119,361, 8,119,381, 8,124,369, and 8,129,134, which are specifically incorporated by reference.

In certain embodiments, any of the nucleases, including the modified nucleases as described herein, may be used in the methods, compositions, and kits according to the invention. In particular embodiments, nuclease activity of an unmodified nuclease may be compared with nuclease activity of any of the modified nucleases as described herein, e.g. to compare for instance off-target or on-target effects. Alternatively, nuclease activity (or a modified activity as described herein) of different modified nucleases may be compared, e.g. to compare for instance off-target or on-target effects.

### Other Example Types of Modulating Agents

Also provided herein are compositions for use in carrying out the methods of the invention. More particularly, non-naturally occurring or engineered compositions are provided which comprise one or more of the elements required to ensure genomic perturbation. In particular embodiments, the compositions comprise one or more of the (modified) DNA binding protein, and/or a guide RNA. In particular embodiments, the composition comprises a vector. In an aspect, the vector can target liver macrophages specifically. In an aspect the vector can be configured to target hepatoctyes such that the agent that stimulates macrophage Wnt signaling (modulating agent) is released at the site of injury. In this regard, hepatic delivery is known in the art and can be adapted for the uses as described herein. See, e.g. Mishra et al., BioMed Res Int'l (2013) doi: 10.1155/2013/382184, Huang et al., Bioconjugate Chem. 2017, 28, 2, 283-29; Zimmerman et al., Molecular Therapy, 25: 1, 4 Jan. 2017, Pages 71-78, doi:10.1016/j.ymthe.2003.09.009, incorporated herein by reference.

In further particular embodiments, the vector comprises a polynucleotide encoding a gRNA. In particular embodiments, the vector comprises two or more guide RNAs. The two or more guide RNAs may target a different target (so as to ensure multiplex targeting) or the same target, in which case the different guide RNAs will target different sequences within the same target sequence. Where provided in a vector the different guide RNAs may be under common control of the same promotor, or may be each be under control of the same or different promoters.

In certain embodiments, a modulating agent may comprise silencing one or more endogenous genes. As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

In certain embodiments, a modulant may comprise (i) a DNA-binding portion configured to specifically bind to the endogenous gene and (ii) an effector domain mediating a biological activity.

In certain embodiments, the DNA-binding portion may comprise a zinc finger protein or DNA-binding domain thereof, a transcription activator-like effector (TALE) protein or DNA-binding domain thereof, or an RNA-guided protein or DNA-binding domain thereof.

In certain embodiments, the DNA-binding portion may comprise (i) Cas9 or Cpf1 or any Cas protein described herein modified to eliminate its nuclease activity, or (ii) DNA-binding domain of Cas9 or Cpf1 or any Cas protein described herein.

In some embodiments, the effector domain may be a transcriptional inhibitor (i.e., a repressor domain), such as an mSin interaction domain (SID). SID4× domain or a Krüppel-associated box (KRAB) or fragments of the KRAB domain. In some embodiments the effector domain may be an enhancer of transcription (i.e. an activation domain), such as the VP16, VP64 or p65 activation domain. In some embodiments, the nucleic acid binding portion may be linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal. In some embodiments, the effector domain may be a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity.

### Pharmaceutical Compositions

The present disclosure also provides for pharmaceutical compositions comprising the one or more modulating agents. A “pharmaceutical composition” refers to a composition that usually contains an excipient, such as a pharmaceutically acceptable carrier that is conventional in the art and that is suitable for administration to cells or to a subject.

In yet other embodiments, the methods of the disclosure include administering to a subject in need thereof an effective amount (e.g., therapeutically effective amount or prophylactically effective amount) of the treatments provided herein. Such treatment may be supplemented with other known treatments, such as surgery on the subject. In certain embodiments, the surgery is strictureplasty, resection (e.g., bowel resection, colon resection), colectomy, surgery for abscesses and fistulas, proctocolectomy, restorative proctocolectomy, vaginal surgery, cataract surgery, or a combination thereof.

The term “pharmaceutically acceptable” as used throughout this specification is consistent with the art and means compatible with the other ingredients of a pharmaceutical composition and not deleterious to the recipient thereof.

As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, stabilisers, antioxidants, tonicity controlling agents, absorption delaying agents, and the like. The use of such media and agents for pharmaceutical active components is well known in the art. Such materials should be non-toxic and should not interfere with the activity of the cells or active components.

The precise nature of the carrier or excipient or other material will depend on the route of administration. For example, the composition may be in the form of a parenterally acceptable aqueous solution, which is pyrogen-free and has suitable pH, isotonicity and stability. For general principles in medicinal formulation, the reader is referred to Cell Therapy: Stem Cell Transplantation, Gene Therapy, and Cellular Immunotherapy, by G. Morstyn & W. Sheridan eds., Cambridge University Press, 1996; and Hematopoietic Stem Cell Therapy, E. D. Ball, J. Lister & P. Law, Churchill Livingstone, 2000.

The pharmaceutical composition can be applied parenterally, rectally, orally or topically. Preferably, the pharmaceutical composition may be used for intravenous, intramuscular, subcutaneous, peritoneal, peridural, rectal, nasal, pulmonary, mucosal, or oral application. In a preferred embodiment, the pharmaceutical composition according to the invention is intended to be used as an infuse. The skilled person will understand that compositions which are to be administered orally or topically will usually not comprise cells, although it may be envisioned for oral compositions to also comprise cells, for example when gastro-intestinal tract indications are treated. Each of the cells or active components (e.g., modulants, immunomodulants, antigens) as discussed herein may be administered by the same route or may be administered by a different route. By means of example, and without limitation, cells may be administered parenterally and other active components may be administered orally.

Liquid pharmaceutical compositions may generally include a liquid carrier such as water or a pharmaceutically acceptable aqueous solution. For example, physiological saline solution, tissue or cell culture media, dextrose or other saccharide solution or glycols such as ethylene glycol, propylene glycol or polyethylene glycol may be included.

The composition may include one or more cell protective molecules, cell regenerative molecules, growth factors, anti-apoptotic factors or factors that regulate gene expression in the cells. Such substances may render the cells independent of their environment.

Such pharmaceutical compositions may contain further components ensuring the viability of the cells therein. For example, the compositions may comprise a suitable buffer system (e.g., phosphate or carbonate buffer system) to achieve desirable pH, more usually near neutral pH, and may comprise sufficient salt to ensure isoosmotic conditions for the cells to prevent osmotic stress. For example, suitable solution for these purposes may be phosphate-buffered saline (PBS), sodium chloride solution, Ringer's Injection or Lactated Ringer's Injection, as known in the art. Further, the composition may comprise a carrier protein, e.g., albumin (e.g., bovine or human albumin), which may increase the viability of the cells.

Further suitably pharmaceutically acceptable carriers or additives are well known to those skilled in the art and for instance may be selected from proteins such as collagen or gelatine, carbohydrates such as starch, polysaccharides, sugars (dextrose, glucose and sucrose), cellulose derivatives like sodium or calcium carboxymethylcellulose, hydroxypropyl cellulose or hydroxypropylmethyl cellulose, pregeletanized starches, pectin agar, carrageenan, clays, hydrophilic gums (acacia gum, guar gum, arabic gum and xanthan gum), alginic acid, alginates, hyaluronic acid, polyglycolic and polylactic acid, dextran, pectins, synthetic polymers such as water-soluble acrylic polymer or polyvinylpyrrolidone, proteoglycans, calcium phosphate and the like.

If desired, cell preparation can be administered on a support, scaffold, matrix or material to provide improved tissue regeneration. For example, the material can be a granular ceramic, or a biopolymer such as gelatine, collagen, or fibrinogen. Porous matrices can be synthesized according to standard techniques (e.g., Mikos et al., Biomaterials 14: 323, 1993; Mikos et al., Polymer 35:1068, 1994; Cook et al., J. Biomed. Mater. Res. 35:513, 1997). Such support, scaffold, matrix or material may be biodegradable or non-biodegradable. Hence, the cells may be transferred to and/or cultured on suitable substrate, such as porous or non-porous substrate, to provide for implants.

The pharmaceutical compositions may comprise one or more pharmaceutically acceptable salts. The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. The term “pharmaceutically acceptable salt” further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methyl sulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations. It will be understood that, as used herein, references to specific agents (e.g., neuromedin U receptor agonists or antagonists), also include the pharmaceutically acceptable salts thereof.

Methods of administrating the pharmacological compositions, including agents, cells, agonists, antagonists, antibodies or fragments thereof, to an individual include, but are not limited to, intradermal, intrathecal, intramuscular, intraperitoneal, intravenous, subcutaneous, intranasal, epidural, by inhalation, and oral routes. The compositions can be administered by any convenient route, for example by infusion or bolus injection, by absorption through epithelial or mucocutaneous linings (for example, oral mucosa, rectal and intestinal mucosa, and the like), ocular, and the like and can be administered together with other biologically-active agents. Administration can be systemic or local. In addition, it may be advantageous to administer the composition into the central nervous system by any suitable route, including intraventricular and intrathecal injection. Pulmonary administration may also be employed by use of an inhaler or nebulizer, and formulation with an aerosolizing agent. It may also be desirable to administer the agent locally to the area in need of treatment; this may be achieved by, for example, and not by way of limitation, local infusion during surgery, topical application, by injection, by means of a catheter, by means of a suppository, or by means of an implant.

Therapy or treatment according to the invention may be performed alone or in conjunction with another therapy, and may be provided at home, the doctor's office, a clinic, a hospital's outpatient department, or a hospital. Treatment generally begins at a hospital so that the doctor can observe the therapy's effects closely and make any adjustments that are needed. The duration of the therapy depends on the age and condition of the patient, the stage of the cancer, and how the patient responds to the treatment. Additionally, a person having a greater risk of developing an inflammatory response (e.g., a person who is genetically predisposed or predisposed to allergies or a person having a disease characterized by episodes of inflammation) may receive prophylactic treatment to inhibit or delay symptoms of the disease.

### Delivery of Modulating Agents and Pharmaceutical Compositions

Various delivery systems are known and can be used to administer the agents and pharmacological compositions including, but not limited to, encapsulation in liposomes, microparticles, microcapsules; minicells; polymers; capsules; tablets; and the like. In one embodiment, the agent may be delivered in a vesicle, in particular a liposome. In a liposome, the agent is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,837,028 and 4,737,323. In yet another embodiment, the pharmacological compositions can be delivered in a controlled release system including, but not limited to: a delivery pump (See, for example, Saudek, et al., New Engl. J. Med. 321: 574 (1989) and a semi-permeable polymeric material (See, for example, Howard, et al., J. Neurosurg. 71: 105 (1989)). Additionally, the controlled release system can be placed in proximity of the therapeutic target (e.g., a tumor), thus requiring only a fraction of the systemic dose. See, for example, Goodson, In: Medical Applications of Controlled Release, 1984. (CRC Press, Boca Raton, Fla.).

Delivery of Modulating Agents that are Polynucleotides

In cases the modulating agents are polynucleotides, they may be delivered to cell using suitable methods. In some embodiments, the polynucleotides may be packaged in viruses or particles, or conjugated to a vehicle for delivering into cells.

In some embodiments, the methods include packaging the polynucleotides in viruses and transducing cell with the viruses. Transduction or transducing herein refers to the delivery of a polynucleotide molecule to a recipient cell either in vivo or in vitro, by infecting the cells with a virus carrying that polynucleotide molecule. The virus may be a replication-defective viral vector. In some examples, the viruses may be virus (e.g. retroviruses, replication defective retroviruses, adenoviruses, replication defective adenoviruses, and adeno-associated viruses (AAVs)).

In some examples, the viruses are lentiviruses. Lentiviruses are complex retroviruses that have the ability to infect and express their genes in both mitotic and post-mitotic cells. Examples of lentiviruses include human immunodeficiency virus (HIV) (e.g., strain 1 and strain 2), simian immunodeficiency virus (SIV), feline immunodeficiency virus (FIV), BLV, EIAV, CEV, and visna virus. Lentiviruses may be used for nondividing or terminally differentiated cells such as neurons, macrophages, hematopoietic stem cells, retinal photoreceptors, and muscle and liver cells, cell types for which previous gene therapy methods could not be used. A vector containing such a lentivirus core (e.g. gag gene) can transduce both dividing and non-dividing cells.

In certain embodiments, the viruses are adeno-associated viruses (AAVs). AAVs are naturally occurring defective viruses that require helper viruses to produce infectious particles (Muzyczka, N., Curr. Topics in Microbiol. Immunol. 158:97 (1992)). It is also one of the few viruses that can integrate its DNA into nondividing cells. Vectors containing as little as 300 base pairs of AAV can be packaged and can integrate, but space for exogenous DNA is limited to about 4.5 kb. In some cases, an AAV vector may include all the sequences necessary for DNA replication, encapsidation, and host-cell integration. The recombinant AAV vector can be transfected into packaging cells which are infected with a helper virus, using any standard technique, including lipofection, electroporation, calcium phosphate precipitation, etc. Appropriate helper viruses include adenoviruses, cytomegaloviruses, vaccinia viruses, or herpes viruses. Once the packaging cells are transfected and infected, they will produce infectious AAV viral particles which contain the polynucleotide construct. These viral particles are then used to transduce eukaryotic cells.

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration). Physical methods of introducing polynucleotides may also used. Examples of such methods include injection of a solution containing the polynucleotides, bombardment by particles covered by the polynucleotides, soaking a cell, tissue sample or organism in a solution of the polynucleotides, or electroporation of cell membranes in the presence of the polynucleotides.

Examples of delivery methods and vehicles include viruses, nanoparticles, exosomes, nanoclews, liposomes, lipids (e.g., LNPs), supercharged proteins, cell permeabilizing peptides, and implantable devices. The nucleic acids, proteins and other molecules, as well as cells described herein may be delivered to cells, tissues, organs, or subjects using methods described in paragraphs [00117] to [00278] of Feng Zhang et al., (WO2016106236A1), which is incorporated by reference herein in its entirety.

In some cases, the methods include delivering the barcode construct and/or another element (e.g., a perturbation element) to cells. In such cases, the barcode construct and/or another element (e.g., a perturbation element) may be RNA molecules.

### Organs and Tissues

As used herein, “organ” means a collection of tissues joined into structural unit to serve a common function. Examples of organs include, but are not limited to, skin, sweat glands, sebaceous glands, mammary glands, bone, brain, hypothalamus, pituitary gland, pineal body, heart, blood vessels, larynx, trachea, bronchus, lung, lymphatic vessel, salivary glands, mucous glands, esophagus, stomach, gallbladder, liver, pancreas, small intestine, large intestine, colon, urethra, kidney, adrenal gland, conduit, ureter, bladder, fallopian tube, uterus, ovaries, testes, prostate, thyroid, parathyroid, meibomian gland, parotid gland, tonsil, adenoid, thymus, and spleen. In one example, the organ is liver. In certain examples, the organ is liver, spleen, intestine, colon, bone marrow, an immune tissue or organ, or a tissue or organ of the gastrointestinal track. In certain cases, the organ or tissue is an organ or tissue of the immune system, e.g., lymphoid organs such as bone marrow, thymus, lymph nodes, spleen, tonsils, other specialized tissues in the mucous membranes of the body, e.g., the bowel. In certain cases, the organ or tissue is a part of the gastrointestinal track, e.g., pharynx, esophagus, stomach, duodenum, small intestine, large intestine.

As used herein, “tissue” means an aggregate of cells. Examples of tissues include, but are not limited to, connective tissue (e.g., areolar connective tissue, dense connective tissue, elastic tissue, reticular connective tissue, and adipose tissue), muscle tissue (e.g., skeletal muscle, smooth muscle and cardiac muscle), genitourinary tissue, gastrointestinal tissue, pulmonary tissue, bone tissue, nervous tissue, and epithelial tissue (e.g., simple epithelium and stratified epithelium), endoderm-derived tissue, mesoderm-derived tissue, and ectoderm-derived tissue.

As used herein, the one or more genes may be involved in functions, growth, proliferation, and generation of stem cells. As used herein, “stem cell” means a cell that exhibits potency and self-renewal. Stem cells include, but are not limited to, totipotent cells, pluripotent cells, multipotent cells, oligopotent cells, unipotent cells, and progenitor cells. Stem cells may be embryonic stem cells, peri-natal stem cells, adult stem cells, amniotic stem cells, and induced pluripotent stem cells.

### Injuries and Diseases

The methods herein may be used for treating various injuries and diseases, e.g., an acute injury, a chronic injury, injury is caused by an metabolic insult (e.g., high fat diet), a chronic disease, or a liver disease.

The injury herein may be an acute injury. As used herein, the term “acute injury” includes injuries that have occurred suddenly or recently occurred. For example, an acute injury may have occurred suddenly, e.g., due to a traumatic event (external or internal), infections (e.g., caused by bacterial viruses, fungi and parasites), stroke (cerebral circulatory disturbance and intracerebral or subarachnoid haemorrhage), intoxications, and traumatic lesions. The injury herein may be a chronic injury or disease. As used herein, the term “chronic injury” an injury disease that has a slow, insidious onset and generally a long duration.

The methods herein may be used for treating various diseases. In some cases, the disease is metabolic diseases such as obesity, as well as related disorders such as eating disorder, cachexia, diabetes mellitus, hypertension, coronary heart disease, hypercholesterolemia, dyslipidemia, osteoarthritis, gallstones, and sleep apnea, and disorders related to ROS defense, such as diabetes mellitus, neurodegenerative disorders, and cancer, e.g. cancers of the reproductive organs, high blood pressure, hypertension, high blood cholesterol, dyslipidemia, type 2 diabetes, insulin resistance, glucose intolerance, hyperinsulinemia, coronary heart disease, angina pectoris, congestive heart failure, stroke, gallstones, cholecystitis and cholelithiasis, gout, osteoarthritis, obstructive sleep apnea and respiratory problems, some types of cancer (such as endometrial, breast, prostate, and colon), complications of pregnancy, poor female reproductive health (such as menstrual irregularities, infertility, irregular ovulation), bladder control problems (such as stress incontinence); uric acid nephrolithiasis; psychological disorders.

In some examples, the injury or disease is a liver injury or disease. Examples of liver injuries or disease include nonalcoholic steatohepatitis, alcoholic hepatitis, and Reye's Syndrome, liver disorders or injuries caused by trauma, intoxication, in particular by alcohol, drugs or food intoxication, radiation, infection, cholestasis, immune reactions, inherited liver diseases and inherited metabolic liver diseases, cirrhosis, alcoholic and nonalcoholic liver disease, chronic hepatitis, Wilson's Disease, and heamochromatosis, liver diseases caused by alcohol (e.g. ASH), non-alcoholic fatty liver changes (such as NAFLD including NASH), nutrition-mediated liver injury (for example starvation), other toxic liver injury (such as unspecific hepatitis induced by e.g. drugs such as but not limited to acetaminophen (paracetamol), chlorinated hydrocarbons (e.g. CCl4), amiodarone (cordarone), valproate, tetracycline (only i.v.), isoniacid, or food intoxication resulting in acute or chronic liver failure, e.g. by consumption of mushrooms containing aflatoxins or ingestion of certain metal (such as copper or cadmium) or herbal products used in natural medicine (homeopatics such as Milk thistle, Chaparral, Kawa-Kawa), interference of bilirubin metabolism, hepatitis like syndromes, cholestasis, granulomatous lesions, intrahepatic vascular lesions and cirrhosis), trauma and surgery (e.g. Pringle maneuver), radiation-mediated liver injury (such as caused by radiotherapy), inflammatory liver disease [caused e.g. by hepatitis B virus (HBV) and hepatitis C virus (HCV) infections] and autoimmune-mediated liver disease (e.g. autoimmune hepatitis), injury due to sepsis, genetic liver disorders (such as heamochromatosis and alpha1 antitrypsin deficiency), and other inherited metabolic liver diseases, e.g. metabolic steatohepatitis (MSH).

Liver diseases or injuries may also encompass liver cancer. The term “liver cancer” within the meaning of the invention includes carcinomas in the liver, hepatocellular carcinoma (HCC), metastases in liver originated from any organ (e.g. colon, breast), cholangiocarcinoma, in which epithelial cell components of the tissue are transformed resulting in a malignant tumor, subtypes of the mentioned disorders, e.g., liver cancers characterized by intracellular proteinaceous inclusion bodies, HCCs characterized by hepatocyte steatosis, and fibrolamellar HCC. For example, precancerous lesions are also included such as those characterized by increased hepatocyte cell size (the “large cell” change), and those characterized by decreased hepatocyte cell size (the “small cell” change) as well as macro regenerative (hyperplastic) nodules. Liver disease is further understood to comprise hyperproliferative diseases of liver e.g. benign liver neoplasms such as liver cell adenoma and/or focular nodular hyperplasia (FNH).

In some embodiments, the diseases also include cancers. Examples of cancers include liquid tumors such as leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, or multiple myeloma, solid tumors such as sarcomas and carcinomas. Examples of solid tumors include, but are not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, epithelial carcinoma, bronchogenic carcinoma, hepatoma, colorectal cancer (e.g., colon cancer, rectal cancer), anal cancer, pancreatic cancer (e.g., pancreatic adenocarcinoma, islet cell carcinoma, neuroendocrine tumors), breast cancer (e.g., ductal carcinoma, lobular carcinoma, inflammatory breast cancer, clear cell carcinoma, mucinous carcinoma), ovarian carcinoma (e.g., ovarian epithelial carcinoma or surface epithelial-stromal tumour including serous tumour, endometrioid tumor and mucinous cystadenocarcinoma, sex-cord-stromal tumor), prostate cancer, liver and bile duct carcinoma (e.g., hepatocelluar carcinoma, cholangiocarcinoma, hemangioma), choriocarcinoma, seminoma, embryonal carcinoma, kidney cancer (e.g., renal cell carcinoma, clear cell carcinoma, Wilm's tumor, nephroblastoma), cervical cancer, uterine cancer (e.g., endometrial adenocarcinoma, uterine papillary serous carcinoma, uterine clear-cell carcinoma, uterine sarcomas and leiomyosarcomas, mixed mullerian tumors), testicular cancer, germ cell tumor, lung cancer (e.g., lung adenocarcinoma, squamous cell carcinoma, large cell carcinoma, bronchioloalveolar carcinoma, non-small-cell carcinoma, small cell carcinoma, mesothelioma), bladder carcinoma, signet ring cell carcinoma, cancer of the head and neck (e.g., squamous cell carcinomas), esophageal carcinoma (e.g., esophageal adenocarcinoma), tumors of the brain (e.g., glioma, glioblastoma, medulloblastoma, astrocytoma, medulloblastoma, craniopharyngioma, ependymoma, pinealoma, hemangioblastoma, acoustic neuroma, oligodendroglioma, schwannoma, meningioma), neuroblastoma, retinoblastoma, neuroendocrine tumor, melanoma, cancer of the stomach (e.g., stomach adenocarcinoma, gastrointestinal stromal tumor), or carcinoids. Lymphoproliferative disorders are also considered to be proliferative diseases, cancers in which expression of an EMT program e.g., breast cancer, colon cancer, lung cancer, prostate cancer, testicular cancer, brain cancer, skin cancer, rectal cancer, gastric cancer, esophageal cancer, tracheal cancer, head and neck cancer, pancreatic cancer, liver cancer, ovarian cancer, lymphoid cancer, cervical cancer, vulvar cancer, melanoma, mesothelioma, renal cancer, bladder cancer, thyroid cancer, bone cancers, carcinomas, sarcomas, and soft tissue cancers.

### Methods of Identifying Target Genes

The present disclosure also includes identifying genes that can be used as target for treating the injuries and diseases. In general, the method include determining expression of one or more genes from single cells in an organ at a first time point and a second time point; selecting a first subset of genes from the one or more genes, wherein expression of the first subset of genes at the first and the second time points are different; determining spatial locations of cells expressing the first subset of genes in the organ at the first and the second time points by an in situ hybridization assay; and selecting a second subset of genes based on the spatial locations of the cells expressing the second subset of genes.

### Gene Profiling in Single Cells

In some embodiments, the methods herein include determining expression of one or more genes from single cells in an organ over a time course. In some cases, the expression of the gene(s) at a first time point and a second time point may be determined and compared. Subject of genes whose expressions alters at different time points may be selected. In some examples, the time points may be two or more of: a time point before the injury, a time point in an injury phase, a time point in a recovery phase, a time point in a proliferative phase, and a time in a termination phase.

In some examples, the expression of the gene(s) may be determined by single cell gene expression profiling. In certain examples, such methods may include separating, detecting and/or quantifying markers at the nucleic acid level, more particularly RNA level, e.g., at the level of hnRNA, pre-mRNA, mRNA, or cDNA. Standard quantitative RNA or cDNA measurement tools known in the art may be used. Non-limiting examples include hybridisation-based analysis, microarray expression analysis, digital gene expression profiling (DGE), RNA-in-situ hybridisation (RISH), Northern-blot analysis and the like; PCR, RT-PCR, RT-qPCR, end-point PCR, digital PCR or the like; supported oligonucleotide detection, pyrosequencing, polony cyclic sequencing by synthesis, simultaneous bi-directional sequencing, single-molecule sequencing, single molecule real time sequencing, true single molecule sequencing, hybridization-assisted nanopore sequencing, sequencing by synthesis, single-cell RNA sequencing (sc-RNA seq), or the like. By means of an example, methods to profile the RNA content of large numbers of individual cells have been recently developed. To do so, special microfluidic devices have been developed to encapsulate each cell in an individual drop, associate the RNA of each cell with a ‘cell barcode’ unique to that cell/drop, measure the expression level of each RNA with sequencing, and then use the cell barcodes to determine which cell each RNA molecule came from.

In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the invention involves high-throughput single-cell RNA-seq and/or targeted nucleic acid profiling (for example, sequencing, quantitative reverse transcription polymerase chain reaction, and the like) where the RNAs from different cells are tagged individually, allowing a single library to be created while retaining the cell identity of each read. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; and Gierahn et al., “Seq-Well: portable, low-cost RNA sequencing of single cells at high throughput” Nature Methods 14, 395-398 (2017), all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; and International patent application number PCT/US2016/059239, published as WO2017164936 on Sep. 28, 2017, which are herein incorporated by reference in their entirety.

As discussed, while the platform has been optimized for the generation of individually barcoded single-cell sequencing libraries following confinement of cells and mRNA capture beads (Macosko, E. Z., Basu, A., Satija, R., Nemesh, J., Shekar, K., Goldman, M., Tirosh, I., Bialas, A. R., Kamitaki, N., Martersteck, E. M., Trombetta, J. J., Weitz, D. A., Sanes, J. A., Shalek, A. K., Regev, A., McCarroll, S. A. “Highly Parallel Genome-wide Expression Profiling of Individual Cells Using Nanoliter Droplets” Cell, 161, 1202-1214), it is capable of multiple levels of data acquisition. The platform is compatible with other assays and measurements performed with the same array. For example, profiling of human antibody responses by integrated single-cell analysis is discussed with regard to measuring levels of cell surface proteins (Ogunniyi, A. O., B. A. Thomas, T. J. Politano, N. Varadarajan, E. Landais, P. Poignard, B. D. Walker, D. S. Kwon, and J. C. Love, “Profiling Human Antibody Responses by Integrated Single-Cell Analysis” Vaccine, 32(24), 2866-2873.) The authors demonstrate a complete characterization of the antigen-specific B cells induced during infections or following vaccination, which enables and informs one of skill in the art how interventions shape protective humoral responses. Specifically, this disclosure combines single-cell profiling with on-chip image cytometry, microengraving, and single-cell RT-PCR.

### Seq-Well

In some embodiments, the expression of the one or more genes may be determined using a method comprising a first functionalized surface of each well or container, wherein the functionalized surface comprises an affinity resin; and a second functionalized surface, such as a top surface of an array material, wherein the functionalized surface provides accessible ionic functional groups. The second surface is configured to be sealed, for example with a permeable membrane, as described herein. According to the invention, the well or container is loaded with one or more cells, such as 1, 2, 3, 4, 5 or more cells of a cell or tissue sample, together with a detection agent, and sealed, for example with a permeable membrane as described herein. In an embodiment of the invention, the detection agent is attached to a barcoded bead. In an embodiment of the invention, the detection reagent is selected to bind to a peptide or nucleic acid. In certain example embodiments, additional reagents may be preloaded into the well or container before sealing. Such reagents can include, without limitation, DNA and/or RNA amplification reagents, polymerases, reverse transcriptase, nucleases, enzymes, antigen binding proteins, labeling reagents, and the like.

In one embodiment, the diagnostic is configured to detect one or more mutated nucleic acids, for example by amplification based methods and/or sequencing. For example, reverse transcription PCR (RT-PCR) can be used to detect mutations in transcribed genes. Additionally, any sequencing technique can be used to determine the presence of a mutation. The present invention also provides for a kit that includes primers that are specific to sequences encompassing the mutations.

In some examples, the method include Seq-Well. An example of the Seq-Well approach is described in Gierahn et al., Nature Methods 2017. In some examples, Seq-Well assays may be performed as described in PCT/US2018/057170, incorporated herein by reference.

Membrane Preparation: (1.) Place a pre-cut (22×66 mm) polycarbonate membrane onto a glass slide, carefully using a gloved finger and tweezers to separate the membrane and paper (Note 1: orientation of polycarbonate membranes not important; discard any membranes that have creases or large-scale imperfections). (2.) Place membranes onto a shelf in the plasma cleaner (Note: Place membranes on bottom shelf to reduce risk of them flying after vacuum is removed. (3) Close the plasma cleaner door, and then turn on the main power and pump switch. To form a vacuum, ensure that the 3-way valve lever is at the 9:00 position as shown below. (4.) Allow vacuum to form for 2 minutes. Once the vacuum has formed, simultaneously turn the valve to 12:00 while turning the power to the Hi setting (shown below). Note: plasma (pink) form should be observed. (Note: The plasma should be a bright pink. If not, adjust the air valve to increase or decrease the amount of oxygen you are letting into the chamber.) (5) Treat membranes with plasma for 7 minutes. (6) Critical—After treatment, in the following order, turn the RF level valve from HIGH to OFF, then turn off the power followed by turning off the vacuum. Then slowly open the valve until you can barely hear air entering chamber (Approximate valve position shown below). Leave until door opens (˜5 min). (7.) Remove slides from plasma cleaner and transfer to a 4 well plate. (Note 1: if membranes have slightly folded over, Slowly flip the membrane back using needle nosed tweezers; Note 2: if membranes have blown off the slide entirely, repeat above procedure to ensure you know which side was exposed to plasma.) (8.) Quickly pipet 5 mL of 1×PBS over the dry membrane, preventing the membrane from folding on itself (Note: gently hydrate one end of the membrane with a single drop so that it adheres to the slide before dispensing the entire volume) (9.) Remove any air bubbles underneath the membrane using wafer forceps. (10.)

Membranes are now functionalized and ready for use. (Note 1: membranes solvated with 1×PBS should be used same day; Note 2: if transporting solvated membranes (e.g. between buildings), remove all by ˜1 mL of PBS to prevent membranes from flipping within the dish; note 3: Alternatively, membranes can be solvated in a 2% Ficoll solution and stored dry for 2 weeks at room temperature.) When ready to use membranes, can be rehydrated with 1×PBS.

Bead Loading: (1.) Aspirate storage solution and solvate arrays with 5 mL of bead loading buffer (BLB). (2.) Place arrays under vacuum with rotation (50 rpm) for 10-15 minutes to remove air bubbles in wells. (3.) Aliquot 110,000 beads from stock into a 1.5 mL tube and spin on a tabletop centrifuge for 10-15 seconds to form a pellet. (4.) Aspirate storage buffer and wash beads once in 500 μL of BLB. (5.) Pellet beads, aspirate BLB, and resuspend beads in 200 μL of BLB. (Note: for each array, recommended to load ˜110,000 beads, e.g., when running two arrays you would aliquot 220,000 beads, wash, and re-suspend in 400 μL of BLB.) (6.) Before loading beads, thoroughly aspirate BLB from the dish containing the array, being careful not to aspirate or dry the PDMS surface of the array. (7.) Use a 200 μL pipette to apply 200 μL containing 110,000 beads, in a drop-wise fashion, to the surface of the array. (8.) Place the loaded array(s) onto a rotator for 10 minutes (75 rpm). (9.) Thoroughly wash arrays to remove excess beads from the surface. For each wash: (a.) Position the array so that it sits in the center of the 4-well dish. (b.)

Dispense 500 μL of BLB in the upper right corner of the array and 500 μL in the bottom right corner of the array (careful not to directly pipette onto the microwells, as it can dislodge beads). (c.) Using wafer forceps, push the array against the left side of the 4-well dish to create a capillary flow, which will help remove beads from the surface. (d.) Aspirate the liquid, reposition the array, and repeat on the opposite side. (10.) Repeat step 9 as necessary. Periodically examine the array under microscope to verify that no loose beads are present on the surface, as this will interfere with membrane attachment. (11.) Once excess beads have been removed from the surface, solvate the array with 5 mL of BLB and proceed to cell loading. (Notes: If continuing to cell loading immediately (i.e., within 1-5 hours), loaded arrays should be stored in 5 mL of BLB. Loaded arrays can be stored for up to 72 hours in Array Quenching Buffer.

Cell Loading (without imaging): (1) Arrays should be loaded with beads and immersed in BLB. (2) Obtain a cell or tissue sample and prepare a single cell suspension using your preferred protocol (3) While preparing your single cell suspension, aspirate the BLB from array and soak it in 5 mL of RPMI+10% FBS for 5 minutes (4) After obtaining a single cell suspension, count cells using a hemocytometer and make a new solution of 10,000 cells in 200 μL of RPMI+10% FBS (Cell Loading Solution) (5) Aspirate the RPMI+10% FBS solution, center the array in well, then load the cell loading solution in a dropwise fashion onto the surface of the array (6) Intermittently rock the array in the x & y direction for 5 minutes (to visualize membrane sealing or cell loading, pre-label cells with AF647-anti CD45 if leukocytes or another surface marker in AF647) (7) Wash arrays 4× with 5 mL of PBS to remove FBS in media—this is critical to ensure successful membrane attachment (Aspirate final PBS wash and replace with 5 mL of RPMI media (no FBS).

Cell Loading (with imaging): (1) When pre-imaging cells, cells should be loaded first as beads will obstruct view of many cells and bead autofluorescence can interfere with the signal (2) Obtain a cell or tissue sample and prepare a single cell suspension using your preferred protocol (3) Count cells using a hemocytometer and resuspend 10,000 cells in 200 μL of cold CellCover (Anacyte). (4) Incubate cells at 4° C. for 1 hour (5) After the cells have been fixed, perform antibody staining at 4° C. (Note: Some epitopes may no longer be available as a result of the fixation process) (6) Wash cells twice with 1×PBS, resuspend in 200 μL of CellCover10 buffer (pH 10+10% FBS) and place on ice. (7) Obtain empty functionalized array(s), aspirate storage solution and soak array(s) with 5 mL of CellCover10 buffer (8) Aspirate media and load fixed cells onto the array(s) in a dropwise format (9) Gently rock the array(s) in the x & y direction for 5 minutes (10) Wash array(s) twice with 5 mL of CellCover10 (pH 10+10% FBS), then solvate in 5 mL of CellCover (No FBS). (11) Place a lift slip onto the array(s), then image with a microscope (12) After imaging, wash array(s) in 5 mL of CellCover10 media (13) Immediately load beads using the bead loading protocol provided above (14) Proceed with membrane sealing.

Membrane Sealing: (1) Use wafer forceps to transfer the array(s) from media to the lid of a 4-well dish, being careful to keep the array as close to horizontal as possible (2) Use wafer forceps to remove a pre-treated membrane from the 4-well dish. (3) Gently dab away moisture from the glass slide on the paper towel until the membrane does not spontaneously change position on the glass slide (4) Carefully position the membrane on the center of the microscope slide, leaving a small (2-3 mm) membrane overhang beyond the edge of slide (5) Holding the membrane in your left hand, invert the microscope slide so that the treated surface is facing down (6) Place the overhang of the membrane in contact with the PDMS surface of the array just above the boundary of the microwells (7) Using your right hand, firmly hold down the overhang of the membrane against the PDMS surface of the array (8) Note: While maintaining pressure with your right hand to hold the membrane in place, gently apply the membrane; for optimal results, use little to no pressure while applying the membrane with the left hand; Attempts to manually seal the microwell device using pressure result in a ‘squeegee’ effect, effectively removing moisture from the membrane while fixing membrane creases in place). (9) After applying the membrane, carefully pry the array and membrane from the surface of the lid and transfer to an Agilent clamp (10) Tighten clamp to the point of resistance and place in a 37° C. incubator for 30 minutes (11) Repeat membrane sealing procedure if running multiple arrays.

Cell Lysis and Hybridization: (1) Remove the clamp from the incubator, and then remove the array from the Agilent clamp (2) Submerge the array with top slide still attached in 5 mL of pre-lysis buffer (5 M Guanidine thiocyanate and 1 mM EDTA) (3) Gently rock the array in pre-lysis buffer until the top glass slide lifts off (Note: time necessary for detachment of the top slide varies (10 seconds-5 minutes) (4) Once the top slide has detached, aspirate the pre-lysis buffer and add 5 mL of complete lysis buffer to the array (Note: Alternatively, 5 mL of complete lysis buffer can be prepared by adding 25 μL of 20% Sarkosyl and 50 μL of Beta-mercaptoethanol to pre-lysis buffer; use a separate waste container for lysis buffer because guanidine thiocyanate can react with bleach in TC traps to create toxic gas (5) Rock the array for 20 minutes at 50-60 rpm (6) Remove the lysis buffer and wash once with 5 mL of hybridization buffer. (7) Aspirate hybridization buffer and add another 5 mL of hybridization buffer to the array and rock for 40 minutes at 50-60 rpm (8) While array are rocking in hybridization buffer, prepare reverse transcription master mix.

Bead Removal: (1) Aspirate hybridization buffer and replace with 5 mL of wash buffer (2) rock for 3 min (3) remove membrane with fine-tipped tweezers (4) identify orientation of a lifter slip such that feet are facing upwards (5) place lifter slip(s) in a separate 4-well dish with feet oriented upwards (6) carefully transfer the array(s) to the new dish, inverting the array(s) so that the PDMS surface is in contact with the feet of the lift slips (7) transfer 3 mL of wash buffer to the dish containing the inverted array(s) (8) precisely (+/−2 grams) weigh the dish containing inverted array(s) to properly balance the centrifuge (9) Spin for 5 minutes at 1000×G. (10) After centrifugation, collect the beads and transfer them to a 15 mL conical tube for each array, if running multiple: (a) rinse the glass slide on the back of the array with wash buffer (b) invert the array and rinse the PDMS surface (c) lightly scrape the surface of the array to remove any retained beads using a microscope slide (d) remove array and rinse both sides of the lifter slips (e) collect suspended beads (10-12 mL) and transfer to a 15-mL conical tube. (11) Spin conical tube(s) for 5 minutes at 3000×G. (12) aspirate all liquid but 1 mL and transfer beads to a clean 1.5 mL centrifuge tube (13) Rinse 15 mL conical with 500 μL of wash buffer and add to the 1.5 mL tube.

### Graph-Based Clustering of Single-Cell Transcriptomes

In some cases, expression data may be analyzed. For single cell-clustering analyses, an example approach is similar to the recently proposed clustering strategy for Drop-Seq data. Briefly, as in Macosko et al. (Macosko, E. Z., Basu, A., Satija, R., Nemesh, J., Shekar, K., Goldman, M., Tirosh, I., Bialas, A. R., Kamitaki, N., Martersteck, E. M., Trombetta, J. J., Weitz, D. A., Sanes, J. A., Shalek, A. K., Regev, A., McCarroll, S. A. “Highly Parallel Genome-wide Expression Profiling of Individual Cells Using Nanoliter Droplets” Cell, 161, 1202-1214. In some examples, first the set of genes that is most variable across the dataset is identified, after controlling for the relationship in single-cell RNA-seq data that inherently exists between mean expression and variability by binning genes into 20 bins based on their average expression level, and z-scoring dispersion (mean/variance) estimates within a bin. Genes which are detected in less than 2.5% of PBMCs (5% of monocytes for the Mtb experiments) are excluded, and a dispersion cutoff of 0.5 is used to select variable genes, resulting in the selection of 687 variable genes across 4,296 PBMCs and 377 variable genes across 4,638 macrophages.

In some cases, reducing the dimensionality of the dataset is performed, e.g., using principal components analysis. As prescribed in Macosko et al. (Macosko, E. Z., Basu, A., Satija, R., Nemesh, J., Shekar, K., Goldman, M., Tirosh, I., Bialas, A. R., Kamitaki, N., Martersteck, E. M., Trombetta, J. J., Weitz, D. A., Sanes, J. A., Shalek, A. K., Regev, A., McCarroll, S. A. “Highly Parallel Genome-wide Expression Profiling of Individual Cells Using Nanoliter Droplets” Cell, 161, 1202-1214), Applicants ran PCA using the prcomp function in R. Applicants then selected PCs for further downstream analysis (11 PCs in PBMC analysis and 5 PCs in TB Analysis). Markers for distinct cell types may be highly represented among the genes with the largest scores along these PCs. Then t-distributed stochastic neighbor embedding (t-SNE) may be performed using cell loadings for the significant principal components as input, to visualize the structure of the data in two dimensions.

For example, graph-based clustering methods may be used, similar to those that have been recently proposed for both single cell RNA-seq and mass cytometry data (Levine, J. H., et al., “Data-driven phenotypic dissection of AML reveals progenitor-like cells that correlate with prognosis” (2015) Cell 162, 184-197 & Xu, C., and Su., Z. “Identification of cell types from single-cell transcriptomes using a novel clustering method” (2015) Bioinformatics 31(12):1974-1980). A Euclidean distance matrix may be generated on the loadings for the significant principal components as described above, and use this to construct a k-nearest neighbor graph (KNN, k=50 in PBMC analysis, k=40 in TB analysis). The goal may be to identify ‘quasi-cliques’ Xu, C., and Su., Z. “Identification of cell types from single-cell transcriptomes using a novel clustering method” (2015) Bioinformatics 31(12):1974-1980), or ‘communities’ (Levine, J. H., et al., “Data-driven phenotypic dissection of AML reveals progenitor-like cells that correlate with prognosis” (2015) Cell 162, 184-197) of cells that were highly interconnected across this graph. First the KNN graph may be converted into a weighted shared nearest neighbor (SNN) graph, where the weight between any two cells is represented by the percent overlap in their respective K-nearest neighborhoods (Jaccard distance), and pruned low-quality edges with a Jaccard distance of <0.1 (less than 10% overlap in local neighborhoods). Finally, to group the cells into clusters, a method for modularity optimization may be used, which aims to optimize a function describing the density of connections within a cluster versus connections between clusters, essentially to identify highly interconnected nodes within the SNN graph. The smart local moving algorithm may be applied, which is similar to the widely used ‘Louvain’ algorithm for community detection, but implements a local moving heuristic that enables communities to be split up and iteratively re-organized in an attempt to improve the overall partition modularity. This grants the SLM algorithm additional freedom in identifying an optimal clustering solution, and increased sensitivity may be empirically observed and this approach may be consistency applied to single cell data.

### In Situ Hybridization

The methods may further include determining spatial locations of cells expressing the first subset of genes in the organ at the first and the second time points by an in situ hybridization assay; and selecting a second subset of genes based on the spatial locations of the cells expressing the second subset of genes.

The in situ hybridization assay may be fluorescene in situ hybridization (FISH). FISH may refer to a cytogenetic technique used to detect and localize the presence or absence of specific nucleic acid sequences. FISH uses fluorescent probes that bind target sequences to define patterns of gene expression within cells and tissues. The term “FISH probe molecule” refers to a physical probe molecule having a nucleic acid sequence of the oligonucleotide sequence of the FISH probe molecule consisting of a plurality of the in-situ hybridization, for example by FISH provided oligonucleotide sequences for FISH probe molecules has been selected. Although this probe molecules are called FISH probe molecules, they do not necessarily need with fluorescent, but can also be labeled with non-fluorescent markers, for example with chromophores.

In some examples, the in situ hybridization assay may be single molecule FISH (smFISH). In some embodiments, genes or RNA within the tissue or organ is visualized (thus determined spatial location of cells expressing the genes or RNA) using single-molecule fluorescence in-situ hybridization (smFISH) (see Skinner, S. O., et al. Measuring mRNA copy number in individual Escherichia coli cells using single-molecule fluorescent in situ hybridization. Nat. Protoc. 8, 1100-1113 (2013); Lyubimova, A. et al. Single-molecule mRNA detection and counting in mammalian tissue. Nat. Protoc. 8, 1743-1758 (2013); Lubeck, E. & Cai, L. Single-cell systems biology by super-resolution imaging and combinatorial labeling. Nat. Methods 9, 743-748 (2012); Lubeck, E., et al. Single-cell in situ RNA profiling by sequential hybridization. Nat. Methods 11, 360-361 (2014); Ke, R. et al. In situ sequencing for RNA analysis in preserved tissue and cells. Nat. Methods 10, 857-860 (2013); Levesque, M. J., et al. Visualizing SNVs to quantify allele-specific expression in single cells. Nat. Methods 10, 865-867 (2013); and Levesque, M. J. & Raj, A. Single-chromosome transcriptional profiling reveals chromosomal gene expression regulation. Nat. Methods 10, 246-248 (2013)). Description of single molecule FISH can also be found in, for example, Raj A., et al., 2008, “Imaging individual mRNA molecules using multiple singly labeled probes,” Nature Methods 5(10): 877-879; Femino A., et al., 1998, “Visualization of single RNA transcripts in situ,” Science 280: 585-590; Vargas D., et al., 2005, “Mechanism of mRNA transport in the nucleus,” Proc. Natl. Acad. Sci. of USA 102: 17008-17013; Raj A., et al., 2006, “Stochastic mRNA synthesis in mammalian cells,” PLoS Biology 4(10):e309; Maamar H., et al., 2007, “Noise in gene expression determines cell fate in B. subtilis,” Science, 317: 526-529; and Raj A., et al., 2010 “Variability in gene expression underlies incomplete penetrance,” Nature463:913; each of which (including any Supplemental Material) is hereby incorporated by reference herein in its entirety.

In some cases, microscopy technology is used for obtaining and analyzing images obtained from in situ hybridization. For example, super-resolution microscopy is used to visualize one or more labeled transcripts within tissues or organs. Exemplary super-resolution technologies include but are not limited to I⁵M microscopy, 4Pi-microscopy, Stimulated Emission Depletion microscopy (STEDM), Ground State Depletion microscopy (GSDM), Spatially Structured Illumination microscopy (SSIM), Photo-Activated Localization Microscopy (PALM), Reversible Saturable Optically Linear Fluorescent Transition (RESOLFT), Total Internal Reflection Fluorescence Microscope (TIRFM), Fluorescence-PALM (FPALM), Stochastical Optical Reconstruction Microscopy (STORM), Fluorescence Imaging with One-Nanometer Accuracy (FIONA), and combinations thereof. Descriptions of relevant techniques can be found in Chi, 2009 “Super-resolution microscopy: breaking the limits, Nature Methods 6(1):15-18; Blow 2008, “New ways to see a smaller world,” Nature 456:825-828; Hell, et al., 2007, “Far-Field Optical Nanoscopy,” Science 316: 1153; R. Heintzmann and G. Ficz, 2006, “Breaking the resolution limit in light microscopy,” Briefings in Functional Genomics and Proteomics 5(4):289-301; Garini et al., 2005, “From micro to nano: recent advances in high-resolution microscopy,” Current Opinion in Biotechnology 16:3-12; Bewersdorf et al., 2006, “Comparison of I⁵M and 4Pi-microscopy,” 222(2):105-117; and Wells, 2004, “Man the Nanoscopes,” JCB 164(3):337-340; each of which (including Supplemental Material) is hereby incorporated by reference herein in its entirety. In some embodiments, light sheet microscopy (as described in greater detail in the examples set forth herein) is used to visualize one or more labeled (according to any method described herein) or unlabeled aspect of the tissue or its molecular constituents.

The present application also provides aspects and embodiments as set forth in the following numbered Statements:

Statement 1. A method of treating an injury in an organ or tissue, comprising administering to a subject in need thereof an agent that modulates expression and/or activity of one or more genes or gene products that have functions in regulation of proteolysis, chemical homeostasis, secretion by cells, regulation of hydrolase activity, regulation of body fluid levels, homeostatic process, wound healing, glycerolipid metabolic process, response to external stimuli, response to oxygen containing compounds, response to lipid, neutral lipid metabolic process, negative regulation of hydrolase activity, ion homeostasis, response to biotic stimulus, exocytosis, platelet degranulation, response to alcohol, regulated exocytosis, negative regulation of peptidase activity, extracellular matrix, secretory granule, platelet alpha granule, secretory granule lumen, secretory vesicle, vesicle lumen, blood microparticle, intracellular vesicle, extracellular space, cytoplasmic vesicle part, protein lipid complex, enzyme inhibitor activity, enzyme regulator activity, response to hypoxia, apoptosis, complement components functions and activities, or a combination thereof.

Statement 2, An agent that modulates expression and/or activity of one or more genes or gene products that have functions in regulation of proteolysis, chemical homeostasis, secretion by cells, regulation of hydrolase activity, regulation of body fluid levels, homeostatic process, wound healing, glycerolipid metabolic process, response to external stimuli, response to oxygen containing compounds, response to lipid, neutral lipid metabolic process, negative regulation of hydrolase activity, ion homeostasis, response to biotic stimulus, exocytosis, platelet degranulation, response to alcohol, regulated exocytosis, negative regulation of peptidase activity, extracellular matrix, secretory granule, platelet alpha granule, secretory granule lumen, secretory vesicle, vesicle lumen, blood microparticle, intracellular vesicle, extracellular space, cytoplasmic vesicle part, protein lipid complex, enzyme inhibitor activity, enzyme regulator activity, response to hypoxia, apoptosis, complement components functions and activities, or a combination thereof, for use in a method of treating an injury in an organ or tissue.

Statement 3. Use of an agent that modulates expression and/or activity of one or more genes or gene products that have functions in regulation of proteolysis, chemical homeostasis, secretion by cells, regulation of hydrolase activity, regulation of body fluid levels, homeostatic process, wound healing, glycerolipid metabolic process, response to external stimuli, response to oxygen containing compounds, response to lipid, neutral lipid metabolic process, negative regulation of hydrolase activity, ion homeostasis, response to biotic stimulus, exocytosis, platelet degranulation, response to alcohol, regulated exocytosis, negative regulation of peptidase activity, extracellular matrix, secretory granule, platelet alpha granule, secretory granule lumen, secretory vesicle, vesicle lumen, blood microparticle, intracellular vesicle, extracellular space, cytoplasmic vesicle part, protein lipid complex, enzyme inhibitor activity, enzyme regulator activity, response to hypoxia, apoptosis, complement components functions and activities, or a combination thereof, for the manufacture of a medicament for use in a method of treating an injury in an organ or tissue.

Statement 4. The method of Statement 1, or the agent for use according to Statement 2, or the use according to Statement 3, wherein the method further comprises administering to the subject in need thereof another agent that modulates expression and/or activity of one or more genes or gene products that have functions in PPAR signaling pathway, complement and coagulation cascades, PPARa activated gene expression, biological oxidations, metabolism of lipids and lipoproteins, nasopharyngeal carcinoma, intestine probiotics, plasma cell vs plasmablast, liver cancer, liver specific genes, multiple myeloma, response to UVb radiation, heart atrium vs ventricle, aging kidney no blood, endocrine therapy resistance, liver cancer, breast cancer basal, foxa2 targets, stem cell, lung cancer kras, tlx targets, liver cancer subclass g123, liver cancer subclass proliferation, liver cancer stem cell, liver cancer recurrence, liver cancer subclass s3, hepatoblastoma, liver development, liver hnf1a targets, matrisome, liver cancer krt19, fatty acid catabolic process, ammonium ion metabolic process, protein activation cascade, regulation of wound healing, response to estradiol, response to acid chemical, sterol homeostasis, lipoprotein metabolic process, fatty acid beta oxidation, protein maturation, regulation of locomotion, organic hydroxy compound metabolic process, organic acid biosynthetic process, monocarboxylic acid metabolic process, response to inorganic substance, regulation of vesicle mediated transport, regulation of fatty acid metabolic process, organic hydroxy compound transport, defense response, organophosphate ester transport, lipid homeostasis, secretion, anion transport, regulation of lipid biosynthetic process, response to xenobiotic stimulus, regulation of response to external stimulus, small molecule biosynthetic process, regulation of response to external stimulus, regulation of lipid metabolic process, amine metabolic process, autophagy, regulation of secretion, apoptotic signaling pathway, acute inflammatory response, regulation of catabolic process, maintenance of location, regulation of protein secretion, organic acid metabolic process, response to oxygen levels, regulation of cellular ketone metabolic process, organic acid catabolic process, regulation of response to wounding, regulation of extrinsic apoptotic signaling pathway, cellular lipid catabolic process, regulation of reactive oxygen species metabolic process, detoxification, regulation of peptidase activity, organic anion transport, inflammatory response, negative regulation of cell death, fatty acid metabolic process, lipid metabolic process, divalent inorganic cation homeostasis, regulation of endocytosis, alcohol metabolic process, immune response, cellular lipid metabolic process, monocarboxylic acid transport, negative regulation of apoptotic signaling pathway, multicellular organismal homeostasis, organic hydroxy compound biosynthetic process, regulation of cell death, lipid catabolic process, regulation of lipid metabolic process, regulation of steroid metabolic process, regulation of inflammatory response, response to toxic substance, cellular chemical homeostasis, regulation of transport, regulation of lipid catabolic process, regulation of immune effector process, lipid localization, regulation of proteolysis, regulation of secretion, regulation of response to wounding, regulation of multicellular organismal process, cellular homeostasis, single organism catabolic process, response to oxidative stress, behavior, acute phase response, regulation of response to external stimulus, regulation of apoptotic signaling pathway, regulation of cell proliferation, response to reactive oxygen species, endocytic vesicle, endoplasmic reticulum part, endoplasmic reticulum lumen, endoplasmic reticulum, lipid transporter activity, sulfur compound binding, steroid binding, glycosaminoglycan binding, alcohol binding, carboxylic ester hydrolase activity, lipid binding, receptor binding, coenzyme binding, adipogenesis, xenobiotic metabolism, fatty acid metabolism, coagulation, bile acid metabolism, peroxisome, or a combination thereof.

Statement 5. The method of Statement 1 or 4, or the agent for use according to Statement 2 or 4, or the use according to Statement 3 or 4, wherein the method further comprises administering to the subject in need thereof another agent that modulates expression and/or activity of one or more genes or gene products that have functions in HDAC3 targets, photodynamic therapy stress, CEBP targets, tolerant macrophage, response to salirasib, adult tissue stem module, klfl targets, anatomical structure formation involved in morphogenesis, circulatory system process, cellular response to external stimulus, response to wounding, cellular response to extracellular stimulus, cell activation, cellular response to oxygen containing compound, vesicle mediated transport, enzyme linked receptor protein signaling pathway, response to bacterium, regulation of catabolic process, response to ketone, regulation of cell adhesion, response to hormone, blood vessel morphogenesis, response to estrogen, response to radiation, response to extracellular stimulus, cellular response to nitrogen compound, regulation of catalytic activity, vasculature development, response to abiotic stimulus, response to drug, response to growth factor, regulation of protein metabolic process, transmembrane receptor protein tyrosine kinase signaling pathway, cellular response to peptide, hexose metabolic process, cellular response to stress, endocytosis, circulatory system development, response to starvation, hemostasis, response to molecule of bacterial origin, cell surface, peptidase regulator activity, molecular function regulator, peptidase inhibitor activity, phospholipid binding, TNF-a signaling via NFkB, or a combination thereof.

Statement 6. The method of any one of Statements 1 or 4-5, or the agent for use according to any one of Statements 2 or 4-5, or the use according to any one of Statements 3-5, wherein the agent modulates expression and/or activity of one or more genes or gene products in Wnt pathway.

Statement 7. The method of any one of Statements 1 or 4-6, or the agent for use according to any one of Statements 2 or 4-6, or the use according to any one of Statements 3-6, wherein the agent modulates expression and/or activity of one or more genes or gene products that are markers of hepatic stem cells.

Statement 8. The method of any one of Statements 1 or 4-7, or the agent for use according to any one of Statements 2 or 4-7, or the use according to any one of Statements 3-7, wherein the expression and/or activity of the one or more genes or gene products is altered in response to a zone-dependent injury and a zone-independent injury.

Statement 9. The method of any one of Statements 1 or 4-8, or the agent for use according to any one of Statements 2 or 4-8, or the use according to any one of Statements 3-8, wherein the one or more genes or gene products comprises Gclc, Txnrd1, Lars2, Cyp4a14, Apoc2, Apoc1, Cyp2c29, Mt1, Mt2, Saa1, Saa2, Fgl1, Mup17, Mup18, Mup1, Gm23935, mmu-mir-6236, Ly6e, Rnase4, Saa4, Fgl1, Hp, Hpx, Lcn2, Orm1, Apcs, Orm2, Saa1, Saa2, Saa3, Sds, Tacc2, Igfbp1, Cxc11, Thrsp, Serpina3n, Lpin1, Steap4, Mt1, Mt2, Aldh3a2, Cyp2c37, Cyp2c29, Cyp8b1, Ces1d, Apoc1, Hsd17b, Atp5h, Apoc2, Retsat, Mat1a, Angptl3, Chchd10, Hmgcs2, Cyp4a10, Cyp4a14, Gm26917, Lars2, Hyou1, Arrdc3, Mup12, Gm15564, Pdia3, Gm26924, Sephs2, Grip2, Krt8, Krt18, Plin2, Chka, Gclc, Srxn1, Hmox1, S100a8, S100a9, Mup15, Mup4, Ankrd55, Mup11, Mup5, Mup18, Mup9, Mup6, Mup17, Mup19, Alb, Pck1, Slc2a2, F2, Cyp2e1, Glu1, Arg1, Cdh1, Gls2, Ppargc1a, Sox9, Tbx3, Lgr5, Axin2, or a combination thereof.

Statement 9. The method of any one of Statements 1 or 4-8, or the agent for use according to any one of Statements 2 or 4-8, or the use according to any one of Statements 3-8, wherein the one or more genes or gene products are selected from the genes or gene products in any one of Tables 1-8 or in all of Tables 1-8.

Statement 10. The method of any one of Statements 1 or 4-9, or the agent for use according to any one of Statements 2 or 4-9, or the use according to any one of Statements 3-9, wherein the agent induces regeneration of the organ or tissue.

Statement 11. The method of any one of Statements 1 or 4-9, or the agent for use according to any one of Statements 2 or 4-9, or the use according to any one of Statements 3-9, wherein the agent induces functional compensation of the organ or tissue.

Statement 12. The method of any one of Statements 1 or 4-9, or the agent for use according to any one of Statements 2 or 4-9, or the use according to any one of Statements 3-9, wherein the agent induces regeneration and functional compensation of the organ or tissue.

Statement 13. The method of any one of Statements 1 or 4-12, or the agent for use according to any one of Statements 2 or 4-12, or the use according to any one of Statements 3-12, wherein the agent induces generation of cells that compensate function loss caused by the injury in the organ or tissue.

Statement 14. The method of any one of Statements 1 or 4-13, or the agent for use according to any one of Statements 2 or 4-13, or the use according to any one of Statements 3-13, wherein the agent induces cell proliferation in the organ or tissue.

Statement 15. The method of any one of Statements 1 or 4-14, or the agent for use according to any one of Statements 2 or 4-14, or the use according to any one of Statements 3-14, wherein the organ or tissue is liver.

Statement 16. The method of any one of Statements 1 or 4-14, or the agent for use according to any one of Statements 2 or 4-14, or the use according to any one of Statements 3-14, wherein the organ or tissue is spleen.

Statement 17. The method of any one of Statements 1 or 4-14, or the agent for use according to any one of Statements 2 or 4-14, or the use according to any one of Statements 3-14, wherein the organ or tissue is intestine.

Statement 18. The method of any one of Statements 1 or 4-14, or the agent for use according to any one of Statements 2 or 4-14, or the use according to any one of Statements 3-14, wherein the organ or tissue is colon.

Statement 19. The method of any one of Statements 1 or 4-14, or the agent for use according to any one of Statements 2 or 4-14, or the use according to any one of Statements 3-14, wherein the organ or tissue is bone marrow.

Statement 20. The method of any one of Statements 1 or 4-14, or the agent for use according to any one of Statements 2 or 4-14, or the use according to any one of Statements 3-14, wherein the organ or tissue is an immune tissue or organ.

Statement 21. The method of any one of Statements 1 or 4-14, or the agent for use according to any one of Statements 2 or 4-14, or the use according to any one of Statements 3-14, wherein the organ or tissue is a tissue or organ of the gastrointestinal tract.

Statement 22. The method of any one of Statements 1 or 4-21, or the agent for use according to any one of Statements 2 or 4-21, or the use according to any one of Statements 3-21, wherein the injury is an acute injury.

Statement 23. The method of any one of Statements 1 or 4-21, or the agent for use according to any one of Statements 2 or 4-21, or the use according to any one of Statements 3-21, wherein the injury is a chronic injury.

Statement 24. The method of any one of Statements 1 or 4-23, or the agent for use according to any one of Statements 2 or 4-23, or the use according to any one of Statements 3-23, wherein the injury is caused by a metabolic insult.

Statement 25. The method of any one of Statements 1 or 4-23, or the agent for use according to any one of Statements 2 or 4-23, or the use according to any one of Statements 3-23, wherein the injury is caused by a toxic insult.

Statement 26. The method of any one of Statements 1 or 4-24, or the agent for use according to any one of Statements 2 or 4-24, or the use according to any one of Statements 3-24, wherein the injury is caused by high fat diet.

Statement 27. The method of any one of Statements 1 or 4-24, or the agent for use according to any one of Statements 2 or 4-24, or the use according to any one of Statements 3-24, wherein the organ or tissue is liver and the injury is caused by high fat diet.

Statement 28. The method of any one of Statements 1 or 4-23, or the agent for use according to any one of Statements 2 or 4-23, or the use according to any one of Statements 3-23, wherein the injury is caused by a disease.

Statement 29. The method of any one of Statements 1 or 4-23, or the agent for use according to any one of Statements 2 or 4-23, or the use according to any one of Statements 3-23, wherein the injury is caused by a chronic disease.

Statement 30. The method of any one of Statements 1 or 4-23, or the agent for use according to any one of Statements 2 or 4-23, or the use according to any one of Statements 3-23, wherein the injury is caused by an acute disease.

Statement 31. The method of any one of Statements 1, 4-23 or 28-30, or the agent for use according to any one of Statements 2, 4-23 or 28-30, or the use according to any one of Statements 3-2.3 or 28-30, wherein the disease is a liver disease.

Statement 32. The method of Statement 31, or the agent for use according to Statement 31, or the use according to Statement 31, wherein the liver disease is non-alcoholic fatty liver disease, non-alcoholic steatohepatitis, or cirrhosis.

Statement 33. The method of any one of Statements 1 or 4-32, or the agent for use according to any one of Statements 2 or 4-32, or the use according to any one of Statements 3-32, wherein the injury is a zone-independent injury.

Statement 34. The method of any one of Statements 1 or 4-32, or the agent for use according to any one of Statements 2 or 4-32, or the use according to any one of Statements 3-32, wherein the injury is a zone-dependent injury.

Statement 35. The method of Statement 34, or the agent for use according to Statement 34, or the use according to Statement 34, wherein the injury is mainly to or substantially only to pericentral hepatocytes.

Statement 36. The method of Statement 34, or the agent for use according to Statement 34, or the use according to Statement 34, wherein the injury is mainly to or substantially only to periportal hepatocytes.

Statement 37. The method of any one of Statements 1 or 4-36, or the agent for use according to any one of Statements 2 or 4-36, or the use according to any one of Statements 3-36, wherein the subject is a human.

Statement 38. The method of any one of Statements 1 or 4-36, or the agent for use according to any one of Statements 2 or 4-36, or the use according to any one of Statements 3-36, wherein the subject is a non-human animal.

Statement 39. The method of any one of Statements 1 or 4-36, or the agent for use according to any one of Statements 2 or 4-36, or the use according to any one of Statements 3-36, wherein the subject is a non-human mammal.

Statement 40. A method of treating an injury in an organ or tissue, comprising:
determining expression of one or more genes from single cells in the organ or tissue at a first time point and a second time point;
selecting a first subset of genes from the one or more genes, wherein expression of the first subset of genes at the first and the second time points are different;
determining spatial locations of cells expressing the first subset of genes in the organ or tissue at the first and the second time points by an in situ hybridization assay; and
selecting a second subset of genes based on the spatial locations of the cells expressing the second subset of genes
administering an agent that modulates expression and/or activity of one or more of the second subset of genes to a subject in need thereof.

Statement 41. A method of treating an injury in an organ or tissue comprising-administering an agent that modulates expression and/or activity of one or more of a second subset of genes to a subject in need thereof, wherein said second subset of genes has been selected by a method comprising:
determining expression of one or more genes from single cells in the organ or tissue at a first time point and a second time point;
selecting a first subset of genes from the one or more genes, wherein expression of the first subset of genes at the first and the second time points are different;
determining spatial locations of cells expressing the first subset of genes in the organ or tissue at the first and the second time points by an in situ hybridization assay; and
selecting the second subset of genes based on the spatial locations of the cells expressing the second subset of genes.

Statement 42. An agent that modulates expression and/or activity of one or more of a second subset of genes for use in a method of treating an injury in an organ or tissue, wherein said second subset of genes has been selected by a method comprising:
determining expression of one or more genes from single cells in the organ or tissue at a first time point and a second time point;
selecting a first subset of genes from the one or more genes, wherein expression of the first subset of genes at the first and the second time points are different;
determining spatial locations of cells expressing the first subset of genes in the organ or tissue at the first and the second time points by an in situ hybridization assay; and
selecting the second subset of genes based on the spatial locations of the cells expressing the second subset of genes.

### EXAMPLES

### Example 1: High Resolution Characterization of the Liver's Response to Acute Injury

Dynamics of liver regeneration have been studied extensively utilizing toxic insult models, such as APAP, but also relying heavily on the partial hepatectomy model, in which liver mass is surgically resected. These methods can differ dramatically because of the nature of the insult driving the loss of functional tissue mass: APAP overdose results in extreme oxidative stress, mitochondrial dysfunction and zone-dependent damage, whereas zone-independent partial hepatectomy (PH) is simply removal of tissue in a zone-independent manner. (FIG. 1A).

Studies surrounding liver regeneration have primarily focused on the regeneration of lost cellular mass through a proliferative response. Massively-parallel single-cell RNA-sequencing (scRNA-seq) was deployed on thousands of hepatocytes, before, during and after the proliferative phase, in order to assess changes in the transcriptional profile of the liver following zone-dependent (APAP) compared to zone-independent (PH) injury. To add spatial context and validation of the scRNA-seq results, Applicants have coupled this analysis with single molecule fluorescence in situ hybridization (smFISH) in order to measure and quantify the mRNA content of hepatocytes within the mouse liver (FIG. 1A). Applicants have performed these analyses across a dynamic time course in order to assess the transcriptional environment during injury, recovery, and termination phases of liver regeneration (FIG. 1B). This has allowed us to characterize the hepatic regenerative response at a high-resolution revealing the ability of hepatocytes to adapt to decreased functional capacity following a loss of tissue mass due to injury.

To assess the transcriptional response of hepatocytes following acute injury, Applicants applied seq-well, a low-cost, high throughput platform for single-cell mRNA sequencing (scRNA-Seq) (Gierahn et al., Nature Methods 2017) to single cell suspensions obtained from livers from male mice (FIG. 1A, Methods). The seq-well technique relies on gently settling cells into wells by gravity, rather than pairing cells and beads through fluidics, thus reducing harsh processing and making seq-well ideal for fragile and sensitive cell types, such as hepatocytes. After sequencing and estimating gene expression levels, normalization was applied (Methods).

Application of dimensional reduction technique, t-Stochastic Neighbour Embedding (t-SNE), to the dataset revealed a diverse population of cells. While the processing methods enriched for hepatocytes over non-parenchymal cells, some non-parenchymal cells were still present, especially under conditions which experienced immune infiltration (Methods, filter to include hepatocytes only with all samples grouping together in lower PCs, with higher PCs separate by sample. tSNE run to visualize top 13 PCs). Data was subsetted to include only clusters of hepatocytes for further analysis, which revealed distinct separation by condition (FIG. 2a). Interestingly, the introduction of a stressor—acute injury by APAP or PH—forced hepatocytes to become more similar between biological replicates, forming unified clusters in tSNE space for each acute injury condition, as opposed to UT cells, which separated by individual. Without being bound by theory, it is believed that UT variation may be due to the innate heterogeneity of the liver during quiescence and may be influenced by numerous uncontrolled factors. This appears to represent a high level of baseline diversity in the control hepatocytes, but following stimulation, the liver damage response drives the expression patterns to become more similar between animals within a particular condition.

To assess pericentral and periportal gene expression patterns, module scores were calculated for pericentral hepatocyte (PCH) and periportal hepatocyte (PPH) gene lists over the full dataset. Table 1a provides gene expression markers for each treatment (APAP, PH and UT and each time point.

TABLE 1a
Gene expression markers for each treatment (APAP, PH and UT) and each time point.

| | p_val | avg_logFC | pct.1 | pct.2 | p_val_adj | cluster | gene |
|---|---|---|---|---|---|---|---|
| S100a9 | 0 | 1.335714143 | 0.399 | 0.014 | 0 | A6 | S100a9 |
| | | | | | | (APAP | |
| | | | | | | 6 hr) | |
| S100a8 | 0 | 1.116303013 | 0.353 | 0.012 | 0 | A6 | S100a8 |
| Mmp8 | 5.57E−283 | 0.358500766 | 0.136 | 0 | 1.38E−278 | A6 | Mmp8 |
| Srgn | 2.57E−279 | 0.926041531 | 0.284 | 0.014 | 6.38E−275 | A6 | Srgn |
| Hmox1 | 8.93E−278 | 1.29024983 | 0.558 | 0.074 | 2.22E−273 | A6 | Hmox1 |
| Psat1 | 2.17E−268 | 0.300087383 | 0.229 | 0.008 | 5.39E−264 | A6 | Psat1 |
| Il1b | 8.17E−262 | 0.736545761 | 0.236 | 0.009 | 2.03E−257 | A6 | Il1b |
| Srxn1 | 3.54E−259 | 1.874073335 | 0.821 | 0.24 | 8.79E−255 | A6 | Srxn1 |
| Tnfrsf12a | 2.15E−248 | 1.081418539 | 0.623 | 0.112 | 5.33E−244 | A6 | Tnfrsf12a |
| Retnlg | 2.07E−215 | 0.374568433 | 0.129 | 0.002 | 5.13E−211 | A6 | Retnlg |
| Gclc | 1.14E−211 | 1.892741272 | 0.962 | 0.737 | 2.83E−207 | A6 | Gclc |
| Hilpda | 1.84E−209 | 0.769838994 | 0.403 | 0.048 | 4.57E−205 | A6 | Hilpda |
| Hba-a1 | 6.50E−198 | 0.715982212 | 0.165 | 0.005 | 1.61E−193 | A6 | Hba-a1 |
| Clec4d | 8.02E−190 | 0.262215609 | 0.107 | 0.001 | 1.99E−185 | A6 | Clec4d |
| Ppl | 3.63E−182 | 0.82872871 | 0.47 | 0.078 | 9.02E−178 | A6 | Ppl |
| Nid1 | 2.61E−181 | 0.331498174 | 0.229 | 0.015 | 6.49E−177 | A6 | Nid1 |
| Ccl6 | 1.21E−174 | 0.755214086 | 0.274 | 0.024 | 3.00E−170 | A6 | Ccl6 |
| Il1r2 | 3.81E−173 | 0.322216828 | 0.117 | 0.002 | 9.47E−169 | A6 | Il1r2 |
| Cd14 | 2.02E−167 | 0.358694149 | 0.153 | 0.006 | 5.01E−163 | A6 | Cd14 |
| Chka | 5.96E−161 | 1.386835525 | 0.716 | 0.245 | 1.48E−156 | A6 | Chka |
| Plin2 | 2.06E−159 | 1.276258881 | 0.943 | 0.768 | 5.12E−155 | A6 | Plin2 |
| Hba-a2 | 8.30E−156 | 0.532797826 | 0.131 | 0.004 | 2.06E−151 | A6 | Hba-a2 |
| Slc16a6 | 5.60E−149 | 0.864031798 | 0.47 | 0.096 | 1.39E−144 | A6 | Slc16a6 |
| Actb | 3.73E−148 | 0.886001814 | 0.979 | 0.918 | 9.26E−144 | A6 | Actb |
| Fxyd5 | 7.74E−138 | 0.269743073 | 0.138 | 0.006 | 1.92E−133 | A6 | Fxyd5 |
| Txnrd1 | 1.12E−137 | 1.301320776 | 0.838 | 0.551 | 2.77E−133 | A6 | Txnrd1 |
| Cdkn1a | 4.35E−127 | 1.099461278 | 0.702 | 0.285 | 1.08E−122 | A6 | Cdkn1a |
| Pla2g7 | 1.92E−121 | 0.273740007 | 0.119 | 0.005 | 4.77E−117 | A6 | Pla2g7 |
| Mthfd2 | 2.58E−119 | 0.259977893 | 0.205 | 0.02 | 6.41E−115 | A6 | Mthfd2 |
| Krt18 | 5.57E−119 | 1.117912275 | 0.864 | 0.686 | 1.38E−114 | A6 | Krt18 |
| Plek | 2.02E−118 | 0.303053621 | 0.11 | 0.004 | 5.02E−114 | A6 | Plek |
| Krt8 | 2.55E−117 | 1.204053446 | 0.826 | 0.587 | 6.33E−113 | A6 | Krt8 |
| Ptprc | 7.05E−112 | 0.407223706 | 0.153 | 0.011 | 1.75E−107 | A6 | Ptprc |
| Mt1 | 4.09E−110 | 0.931483521 | 0.981 | 0.75 | 1.02E−105 | A6 | Mt1 |
| Tubb6 | 2.80E−105 | 0.533019897 | 0.368 | 0.075 | 6.95E−101 | A6 | Tubb6 |
| Dusp5 | 6.83E−105 | 0.28888562 | 0.196 | 0.021 | 1.70E−100 | A6 | Dusp5 |
| Cidec | 6.14E−101 | 0.306758365 | 0.253 | 0.036 | 1.53E−96 | A6 | Cidec |
| Gsta1 | 5.62E−86 | 0.42042238 | 0.32 | 0.067 | 1.40E−81 | A6 | Gsta1 |
| Lrrc58 | 2.74E−81 | 0.924271525 | 0.711 | 0.444 | 6.81E−77 | A6 | Lrrc58 |
| Rell1 | 8.51E−81 | 0.577000265 | 0.375 | 0.099 | 2.11E−76 | A6 | Rell1 |
| C230081A13Rik | 1.83E−80 | 0.414792324 | 0.279 | 0.055 | 4.54E−76 | A6 | C230081A13Rik |
| Tyrobp | 2.44E−79 | 0.443113689 | 0.189 | 0.026 | 6.06E−75 | A6 | Tyrobp |
| Mt2 | 6.45E−79 | 0.664695984 | 0.938 | 0.619 | 1.60E−74 | A6 | Mt2 |
| Rnf144a | 4.17E−75 | 0.311931338 | 0.177 | 0.023 | 1.03E−70 | A6 | Rnf144a |
| Plscr1 | 5.66E−75 | 0.434306904 | 0.329 | 0.081 | 1.40E−70 | A6 | Plscr1 |
| Alb | 1.63E−71 | 0.418074924 | 1 | 1 | 4.06E−67 | A6 | Alb |
| Cd44 | 1.51E−70 | 0.292904911 | 0.115 | 0.01 | 3.75E−66 | A6 | Cd44 |
| Il1r1 | 1.26E−69 | 0.844988554 | 0.683 | 0.392 | 3.13E−65 | A6 | Il1r1 |
| Cers6 | 2.65E−69 | 0.477445769 | 0.332 | 0.086 | 6.58E−65 | A6 | Cers6 |
| Cd53 | 8.74E−69 | 0.256050768 | 0.103 | 0.008 | 2.17E−64 | A6 | Cd53 |
| Ets2 | 2.70E−68 | 0.627666547 | 0.415 | 0.136 | 6.71E−64 | A6 | Ets2 |
| Myh9 | 1.15E−67 | 0.866093547 | 0.718 | 0.481 | 2.86E−63 | A6 | Myh9 |
| mmu-mir-6240 | 1.30E−66 | 1.082608953 | 0.413 | 0.134 | 3.24E−62 | A6 | mmu-mir-6240 |
| Mrap | 2.88E−66 | 0.826081002 | 0.606 | 0.338 | 7.16E−62 | A6 | Mrap |
| Clec4f | 6.33E−64 | 0.990882766 | 0.339 | 0.095 | 1.57E−59 | A6 | Clec4f |
| Tmsb4x | 2.22E−62 | 0.70513404 | 0.332 | 0.093 | 5.50E−58 | A6 | Tmsb4x |
| Itgb2 | 1.07E−61 | 0.262839863 | 0.105 | 0.01 | 2.66E−57 | A6 | Itgb2 |
| Cd74 | 6.70E−61 | 0.441857606 | 0.155 | 0.022 | 1.66E−56 | A6 | Cd74 |
| Apoa4 | 1.82E−60 | 0.945027313 | 0.721 | 0.49 | 4.53E−56 | A6 | Apoa4 |
| Bcl2l1 | 3.01E−60 | 0.686254569 | 0.501 | 0.228 | 7.48E−56 | A6 | Bcl2l1 |
| H2-Eb1 | 7.87E−60 | 0.321369004 | 0.105 | 0.01 | 1.95E−55 | A6 | H2-Eb1 |
| Fcgr3 | 1.06E−58 | 0.294045394 | 0.107 | 0.011 | 2.64E−54 | A6 | Fcgr3 |
| Serpina3m | 1.07E−58 | 0.606679439 | 0.85 | 0.813 | 2.65E−54 | A6 | Serpina3m |
| Insig2 | 1.68E−58 | 0.683422171 | 0.854 | 0.809 | 4.18E−54 | A6 | Insig2 |
| Vcam1 | 2.58E−58 | 0.437641865 | 0.134 | 0.017 | 6.40E−54 | A6 | Vcam1 |
| Malat1 | 2.86E−58 | 1.108271971 | 0.871 | 0.695 | 7.10E−54 | A6 | Malat1 |
| Ctss | 3.09E−58 | 0.703071213 | 0.212 | 0.043 | 7.66E−54 | A6 | Ctss |
| Cd163 | 4.80E−58 | 0.40657557 | 0.146 | 0.021 | 1.19E−53 | A6 | Cd163 |
| Cd97 | 6.94E−57 | 0.330330436 | 0.115 | 0.013 | 1.72E−52 | A6 | Cd97 |
| Sqstm1 | 9.94E−56 | 0.681739125 | 0.859 | 0.836 | 2.47E−51 | A6 | Sqstm1 |
| Klf6 | 1.97E−55 | 0.612280921 | 0.382 | 0.13 | 4.88E−51 | A6 | Klf6 |
| Cyp4a14 | 2.22E−55 | 0.599586688 | 0.776 | 0.484 | 5.52E−51 | A6 | Cyp4a14 |
| Iqgap1 | 1.16E−54 | 0.434488409 | 0.162 | 0.027 | 2.87E−50 | A6 | Iqgap1 |
| Eif5 | 1.78E−54 | 0.580165484 | 0.816 | 0.756 | 4.42E−50 | A6 | Eif5 |
| H2-Ab1 | 1.51E−52 | 0.449067035 | 0.158 | 0.026 | 3.74E−48 | A6 | H2-Ab1 |
| Rgs2 | 1.81E−49 | 0.380196243 | 0.138 | 0.022 | 4.48E−45 | A6 | Rgs2 |
| 2010003K11Rik | 8.78E−49 | 0.487245683 | 0.396 | 0.15 | 2.18E−44 | A6 | 2010003K11Rik |
| Sat1 | 2.97E−48 | 0.66217907 | 0.47 | 0.222 | 7.37E−44 | A6 | Sat1 |
| Laptm5 | 2.87E−47 | 0.316929935 | 0.124 | 0.018 | 7.13E−43 | A6 | Laptm5 |
| Efhd2 | 1.77E−46 | 0.55453654 | 0.468 | 0.221 | 4.40E−42 | A6 | Efhd2 |
| Trp53inp1 | 9.60E−46 | 0.838899515 | 0.637 | 0.432 | 2.38E−41 | A6 | Trp53inp1 |
| Tubb2a | 2.49E−44 | 0.783817377 | 0.618 | 0.44 | 6.17E−40 | A6 | Tubb2a |
| Nop58 | 1.23E−43 | 0.585927781 | 0.492 | 0.25 | 3.05E−39 | A6 | Nop58 |
| D10Wsu102e | 6.28E−43 | 0.513399976 | 0.37 | 0.148 | 1.56E−38 | A6 | D10Wsu102e |
| Lyz2 | 2.44E−42 | 0.652343138 | 0.234 | 0.065 | 6.06E−38 | A6 | Lyz2 |
| Retsat | 9.48E−42 | 0.480062983 | 0.845 | 0.785 | 2.35E−37 | A6 | Retsat |
| Eif1a | 7.82E−41 | 0.658533359 | 0.599 | 0.41 | 1.94E−36 | A6 | Eif1a |
| Slc39a14 | 7.86E−41 | 0.672079236 | 0.678 | 0.528 | 1.95E−36 | A6 | Slc39a14 |
| C1qb | 9.66E−41 | 0.594567033 | 0.234 | 0.066 | 2.40E−36 | A6 | C1qb |
| Rcan1 | 1.14E−40 | 0.572746322 | 0.425 | 0.191 | 2.82E−36 | A6 | Rcan1 |
| Hnf4a | 1.63E−40 | 0.559027113 | 0.835 | 0.834 | 4.05E−36 | A6 | Hnf4a |
| Hsd17b13 | 2.02E−40 | 0.528446636 | 0.864 | 0.751 | 5.02E−36 | A6 | Hsd17b13 |
| Msn | 2.18E−40 | 0.250996496 | 0.105 | 0.015 | 5.43E−36 | A6 | Msn |
| Sdc4 | 2.72E−40 | 0.441532524 | 0.909 | 0.91 | 6.75E−36 | A6 | Sdc4 |
| Gm20594 | 8.83E−40 | 0.582505706 | 0.442 | 0.195 | 2.19E−35 | A6 | Gm20594 |
| Picalm | 2.41E−39 | 0.642536234 | 0.647 | 0.473 | 5.98E−35 | A6 | Picalm |
| Epas1 | 4.29E−39 | 0.626428547 | 0.604 | 0.426 | 1.07E−34 | A6 | Epas1 |
| Hic2 | 1.51E−38 | 0.26725951 | 0.15 | 0.031 | 3.75E−34 | A6 | Hic2 |
| Cxadr | 6.30E−38 | 0.61841577 | 0.606 | 0.428 | 1.56E−33 | A6 | Cxadr |
| Ddx21 | 7.17E−38 | 0.639844169 | 0.535 | 0.327 | 1.78E−33 | A6 | Ddx21 |
| Agpat9 | 1.20E−37 | 0.296765266 | 0.277 | 0.094 | 2.98E−33 | A6 | Agpat9 |
| Csf1r | 7.16E−37 | 0.643246591 | 0.184 | 0.047 | 1.78E−32 | A6 | Csf1r |
| Rock2 | 3.22E−36 | 0.552475288 | 0.425 | 0.208 | 8.00E−32 | A6 | Rock2 |
| C1qc | 3.82E−36 | 0.54773051 | 0.172 | 0.042 | 9.50E−32 | A6 | C1qc |
| Tra2a | 5.16E−36 | 0.69171846 | 0.337 | 0.138 | 1.28E−31 | A6 | Tra2a |
| Fam134b | 1.18E−35 | 0.620176283 | 0.678 | 0.544 | 2.93E−31 | A6 | Fam134b |
| Cltc | 3.48E−35 | 0.634637641 | 0.661 | 0.519 | 8.64E−31 | A6 | Cltc |
| Gm23388 | 1.61E−34 | 0.421585518 | 0.222 | 0.067 | 4.00E−30 | A6 | Gm23388 |
| Eaf1 | 8.98E−34 | 0.50657558 | 0.427 | 0.221 | 2.23E−29 | A6 | Eaf1 |
| Gcnt2 | 1.97E−33 | 0.461368158 | 0.372 | 0.169 | 4.89E−29 | A6 | Gcnt2 |
| Gm24187 | 2.37E−33 | 0.396868602 | 0.2 | 0.057 | 5.89E−29 | A6 | Gm24187 |
| Mat1a | 3.75E−33 | 0.331703101 | 0.986 | 0.978 | 9.32E−29 | A6 | Mat1a |
| C1qa | 4.44E−33 | 0.471019029 | 0.174 | 0.046 | 1.10E−28 | A6 | C1qa |
| Tjp2 | 3.98E−32 | 0.403000168 | 0.332 | 0.143 | 9.89E−28 | A6 | Tjp2 |
| Lyst | 2.03E−31 | 0.376687839 | 0.31 | 0.124 | 5.03E−27 | A6 | Lyst |
| Ugdh | 2.04E−31 | 0.556346523 | 0.721 | 0.671 | 5.07E−27 | A6 | Ugdh |
| Dgkd | 3.12E−31 | 0.346236575 | 0.282 | 0.109 | 7.74E−27 | A6 | Dgkd |
| Cfp | 6.42E−31 | 0.325079609 | 0.143 | 0.034 | 1.59E−26 | A6 | Cfp |
| Mdn1 | 8.91E−31 | 0.293117859 | 0.251 | 0.088 | 2.21E−26 | A6 | Mdn1 |
| Btg1 | 1.21E−30 | 0.426522166 | 0.356 | 0.163 | 3.00E−26 | A6 | Btg1 |
| Apob | 7.36E−30 | 0.356365368 | 0.99 | 0.989 | 1.83E−25 | A6 | Apob |
| Gnl3 | 1.05E−29 | 0.476957552 | 0.501 | 0.307 | 2.60E−25 | A6 | Gnl3 |
| Ctsl | 1.40E−29 | 0.43840609 | 0.878 | 0.896 | 3.49E−25 | A6 | Ctsl |
| Dot1l | 1.84E−29 | 0.369470561 | 0.282 | 0.112 | 4.56E−25 | A6 | Dot1l |
| 1700017B05Rik | 2.19E−29 | 0.289389909 | 0.255 | 0.094 | 5.43E−25 | A6 | 1700017B05Rik |
| B4galt5 | 2.51E−29 | 0.401953614 | 0.332 | 0.15 | 6.23E−25 | A6 | B4galt5 |
| Asl | 3.01E−29 | 0.434010731 | 0.826 | 0.812 | 7.46E−25 | A6 | Asl |
| Hif1a | 6.99E−29 | 0.471910312 | 0.427 | 0.234 | 1.73E−24 | A6 | Hif1a |
| Ddx3x | 3.19E−28 | 0.48933628 | 0.692 | 0.617 | 7.91E−24 | A6 | Ddx3x |
| Stk40 | 5.19E−28 | 0.487468435 | 0.382 | 0.196 | 1.29E−23 | A6 | Stk40 |
| Jmjd1c | 8.87E−28 | 0.526154806 | 0.47 | 0.278 | 2.20E−23 | A6 | Jmjd1c |
| Ddx5 | 8.92E−28 | 0.473243903 | 0.685 | 0.582 | 2.21E−23 | A6 | Ddx5 |
| Wfdc17 | 1.92E−27 | 0.417330221 | 0.177 | 0.053 | 4.78E−23 | A6 | Wfdc17 |
| Bach1 | 5.63E−27 | 0.672377572 | 0.461 | 0.291 | 1.40E−22 | A6 | Bach1 |
| Ppan | 2.32E−26 | 0.265470715 | 0.255 | 0.1 | 5.77E−22 | A6 | Ppan |
| Vsig4 | 5.27E−26 | 0.454206621 | 0.117 | 0.028 | 1.31E−21 | A6 | Vsig4 |
| Spred1 | 7.20E−26 | 0.335134356 | 0.308 | 0.139 | 1.79E−21 | A6 | Spred1 |
| Chic2 | 8.63E−26 | 0.389971062 | 0.37 | 0.194 | 2.14E−21 | A6 | Chic2 |
| Myo1e | 9.65E−26 | 0.491635416 | 0.475 | 0.296 | 2.40E−21 | A6 | Myo1e |
| Gm26924 | 1.31E−25 | 0.302897605 | 1 | 1 | 3.24E−21 | A6 | Gm26924 |
| Igfbp1 | 2.08E−25 | 0.595262474 | 0.792 | 0.774 | 5.17E−21 | A6 | Igfbp1 |
| Gm15564 | 3.38E−25 | 0.607856981 | 0.983 | 0.977 | 8.41E−21 | A6 | Gm15564 |
| Lpin2 | 7.01E−25 | 0.522681584 | 0.745 | 0.694 | 1.74E−20 | A6 | Lpin2 |
| Cd5l | 7.67E−25 | 0.566281793 | 0.21 | 0.077 | 1.90E−20 | A6 | Cd5l |
| Tsr1 | 8.86E−25 | 0.31396368 | 0.341 | 0.166 | 2.20E−20 | A6 | Tsr1 |
| Creb3l2 | 4.54E−24 | 0.336328333 | 0.329 | 0.158 | 1.13E−19 | A6 | Creb3l2 |
| Ier5 | 5.39E−24 | 0.279704513 | 0.134 | 0.037 | 1.34E−19 | A6 | Ier5 |
| Eef2 | 5.73E−24 | 0.351846239 | 0.9 | 0.908 | 1.42E−19 | A6 | Eef2 |
| Tcerg1 | 5.75E−24 | 0.335808462 | 0.305 | 0.145 | 1.43E−19 | A6 | Tcerg1 |
| Apobec1 | 6.53E−24 | 0.282426929 | 0.239 | 0.094 | 1.62E−19 | A6 | Apobec1 |
| Stat3 | 7.26E−24 | 0.484579314 | 0.604 | 0.472 | 1.80E−19 | A6 | Stat3 |
| Rora | 7.34E−24 | 0.551440037 | 0.549 | 0.429 | 1.82E−19 | A6 | Rora |
| Lars2 | 1.06E−23 | 0.588681759 | 1 | 0.997 | 2.63E−19 | A6 | Lars2 |
| Tars | 1.14E−23 | 0.476757207 | 0.666 | 0.586 | 2.84E−19 | A6 | Tars |
| Xbp1 | 1.76E−23 | 0.440708121 | 0.692 | 0.658 | 4.38E−19 | A6 | Xbp1 |
| Sdad1 | 2.65E−23 | 0.284425559 | 0.289 | 0.129 | 6.58E−19 | A6 | Sdad1 |
| St3gal1 | 6.64E−23 | 0.434367435 | 0.473 | 0.313 | 1.65E−18 | A6 | St3gal1 |
| Son | 1.36E−22 | 0.434214467 | 0.601 | 0.452 | 3.37E−18 | A6 | Son |
| Heatr1 | 1.43E−22 | 0.343295645 | 0.291 | 0.134 | 3.55E−18 | A6 | Heatr1 |
| Ncl | 5.14E−22 | 0.429184065 | 0.69 | 0.629 | 1.28E−17 | A6 | Ncl |
| Iigp1 | 6.99E−22 | 0.611771994 | 0.78 | 0.811 | 1.74E−17 | A6 | Iigp1 |
| Rad54l2 | 7.85E−22 | 0.336619848 | 0.325 | 0.162 | 1.95E−17 | A6 | Rad54l2 |
| Btg2 | 2.09E−21 | 0.538770616 | 0.549 | 0.401 | 5.20E−17 | A6 | Btg2 |
| Tnfrsf1a | 3.42E−21 | 0.453257878 | 0.508 | 0.37 | 8.49E−17 | A6 | Tnfrsf1a |
| Mat2a | 3.46E−21 | 0.469344192 | 0.487 | 0.336 | 8.58E−17 | A6 | Mat2a |
| Itih2 | 4.13E−21 | 0.310907717 | 0.893 | 0.927 | 1.03E−16 | A6 | Itih2 |
| Gtpbp4 | 4.40E−21 | 0.457875649 | 0.477 | 0.321 | 1.09E−16 | A6 | Gtpbp4 |
| Dkc1 | 4.62E−21 | 0.323244532 | 0.315 | 0.159 | 1.15E−16 | A6 | Dkc1 |
| Sdc3 | 4.87E−21 | 0.317989769 | 0.103 | 0.026 | 1.21E−16 | A6 | Sdc3 |
| H3f3b | 5.06E−21 | 0.508752641 | 0.613 | 0.515 | 1.26E−16 | A6 | H3f3b |
| Neat1 | 6.54E−21 | 0.623425327 | 0.31 | 0.158 | 1.62E−16 | A6 | Neat1 |
| Ces1d | 1.34E−20 | 0.385383868 | 0.859 | 0.86 | 3.33E−16 | A6 | Ces1d |
| Gm24245 | 1.85E−20 | 0.573742943 | 0.422 | 0.246 | 4.58E−16 | A6 | Gm24245 |
| Maff | 1.91E−20 | 0.331354317 | 0.224 | 0.095 | 4.75E−16 | A6 | Maff |
| Mfsd2a | 1.92E−20 | 0.423246315 | 0.465 | 0.309 | 4.78E−16 | A6 | Mfsd2a |
| Hnrnpdl | 2.52E−20 | 0.413693336 | 0.406 | 0.246 | 6.25E−16 | A6 | Hnrnpdl |
| Phc2 | 2.87E−20 | 0.309159237 | 0.284 | 0.138 | 7.12E−16 | A6 | Phc2 |
| Dusp16 | 2.97E−20 | 0.415286337 | 0.372 | 0.218 | 7.37E−16 | A6 | Dusp16 |
| Tsku | 3.12E−20 | 0.32057443 | 0.327 | 0.169 | 7.76E−16 | A6 | Tsku |
| Lgmn | 4.82E−20 | 0.436793121 | 0.246 | 0.11 | 1.20E−15 | A6 | Lgmn |
| Etnppl | 6.12E−20 | 0.403812162 | 0.706 | 0.572 | 1.52E−15 | A6 | Etnppl |
| Abca1 | 1.01E−19 | 0.505812774 | 0.604 | 0.512 | 2.51E−15 | A6 | Abca1 |
| Etf1 | 1.42E−19 | 0.447016025 | 0.606 | 0.507 | 3.53E−15 | A6 | Etf1 |
| Pigr | 2.32E−19 | 0.262735133 | 0.947 | 0.941 | 5.76E−15 | A6 | Pigr |
| Ranbp2 | 3.87E−19 | 0.480806807 | 0.566 | 0.451 | 9.61E−15 | A6 | Ranbp2 |
| Myl12a | 5.48E−19 | 0.393725064 | 0.461 | 0.314 | 1.36E−14 | A6 | Myl12a |
| Tiparp | 7.18E−19 | 0.427006824 | 0.346 | 0.193 | 1.78E−14 | A6 | Tiparp |
| Fam107b | 7.48E−19 | 0.422395853 | 0.449 | 0.309 | 1.86E−14 | A6 | Fam107b |
| Gm10800 | 8.12E−19 | 0.538348955 | 0.115 | 0.034 | 2.02E−14 | A6 | Gm10800 |
| Nop56 | 1.10E−18 | 0.250694624 | 0.282 | 0.137 | 2.73E−14 | A6 | Nop56 |
| Tsc22d2 | 1.20E−18 | 0.338155823 | 0.353 | 0.201 | 2.97E−14 | A6 | Tsc22d2 |
| Tmem87b | 1.51E−18 | 0.348646714 | 0.351 | 0.2 | 3.74E−14 | A6 | Tmem87b |
| Jak1 | 1.86E−18 | 0.469606838 | 0.554 | 0.451 | 4.61E−14 | A6 | Jak1 |
| Map2k3 | 3.65E−18 | 0.395195044 | 0.427 | 0.279 | 9.06E−14 | A6 | Map2k3 |
| Stk17b | 1.03E−17 | 0.282498896 | 0.126 | 0.041 | 2.56E−13 | A6 | Stk17b |
| Nolc1 | 1.14E−17 | 0.408444859 | 0.363 | 0.215 | 2.82E−13 | A6 | Nolc1 |
| Fcna | 1.52E−17 | 0.346866554 | 0.124 | 0.04 | 3.77E−13 | A6 | Fcna |
| Eif4g2 | 1.81E−17 | 0.339948168 | 0.757 | 0.774 | 4.50E−13 | A6 | Eif4g2 |
| App | 1.94E−17 | 0.329124793 | 0.315 | 0.17 | 4.83E−13 | A6 | App |
| Gpt2 | 2.10E−17 | 0.368164282 | 0.764 | 0.787 | 5.22E−13 | A6 | Gpt2 |
| Lmo4 | 3.07E−17 | 0.29230743 | 0.308 | 0.169 | 7.63E−13 | A6 | Lmo4 |
| Mrc1 | 4.45E−17 | 0.306341468 | 0.131 | 0.044 | 1.10E−12 | A6 | Mrc1 |
| Mmp14 | 4.96E−17 | 0.259424135 | 0.253 | 0.124 | 1.23E−12 | A6 | Mmp14 |
| Abhd2 | 9.00E−17 | 0.359296116 | 0.37 | 0.221 | 2.24E−12 | A6 | Abhd2 |
| Tatdn2 | 2.25E−16 | 0.309027814 | 0.334 | 0.189 | 5.59E−12 | A6 | Tatdn2 |
| Tbc1d15 | 3.21E−16 | 0.419972242 | 0.461 | 0.337 | 7.98E−12 | A6 | Tbc1d15 |
| Ywhag | 4.64E−16 | 0.464937447 | 0.518 | 0.422 | 1.15E−11 | A6 | Ywhag |
| Dusp6 | 1.09E−15 | 0.476849753 | 0.482 | 0.361 | 2.70E−11 | A6 | Dusp6 |
| Mcl1 | 1.59E−15 | 0.463883705 | 0.573 | 0.468 | 3.94E−11 | A6 | Mcl1 |
| Hspb8 | 1.99E−15 | 0.372641331 | 0.709 | 0.705 | 4.93E−11 | A6 | Hspb8 |
| Epb4.1 | 2.12E−15 | 0.418931034 | 0.537 | 0.431 | 5.25E−11 | A6 | Epb4.1 |
| Kif5b | 3.44E−15 | 0.433974551 | 0.494 | 0.395 | 8.53E−11 | A6 | Kif5b |
| Lrrfip1 | 5.10E−15 | 0.39715369 | 0.37 | 0.237 | 1.27E−10 | A6 | Lrrfip1 |
| Cars | 5.93E−15 | 0.278965519 | 0.329 | 0.192 | 1.47E−10 | A6 | Cars |
| Zyx | 6.80E−15 | 0.285048478 | 0.263 | 0.139 | 1.69E−10 | A6 | Zyx |
| Gnl2 | 1.37E−14 | 0.333718771 | 0.384 | 0.253 | 3.41E−10 | A6 | Gnl2 |
| Fndc3b | 2.02E−14 | 0.297639516 | 0.315 | 0.184 | 5.01E−10 | A6 | Fndc3b |
| Mybbp1a | 2.24E−14 | 0.389680186 | 0.396 | 0.276 | 5.56E−10 | A6 | Mybbp1a |
| Cyp4a10 | 2.95E−14 | 0.309701213 | 0.687 | 0.606 | 7.32E−10 | A6 | Cyp4a10 |
| Ptpn1 | 2.99E−14 | 0.323913522 | 0.353 | 0.212 | 7.42E−10 | A6 | Ptpn1 |
| Rn7sk | 3.96E−14 | 0.324401888 | 0.339 | 0.203 | 9.83E−10 | A6 | Rn7sk |
| Wdr43 | 4.52E−14 | 0.301347392 | 0.353 | 0.221 | 1.12E−09 | A6 | Wdr43 |
| Cybb | 5.80E−14 | 0.363939527 | 0.107 | 0.037 | 1.44E−09 | A6 | Cybb |
| Wbp1l | 5.81E−14 | 0.35324603 | 0.697 | 0.703 | 1.44E−09 | A6 | Wbp1l |
| Plec | 5.98E−14 | 0.355267946 | 0.422 | 0.289 | 1.48E−09 | A6 | Plec |
| Slc20a1 | 9.62E−14 | 0.303408431 | 0.284 | 0.159 | 2.39E−09 | A6 | Slc20a1 |
| Aff4 | 9.83E−14 | 0.483625144 | 0.537 | 0.448 | 2.44E−09 | A6 | Aff4 |
| Tm4sf4 | 1.22E−13 | 0.383397009 | 0.484 | 0.379 | 3.04E−09 | A6 | Tm4sf4 |
| Lcp1 | 1.53E−13 | 0.463298076 | 0.492 | 0.367 | 3.80E−09 | A6 | Lcp1 |
| Ppp1r12a | 1.74E−13 | 0.323279732 | 0.317 | 0.19 | 4.33E−09 | A6 | Ppp1r12a |
| Prrc2c | 1.85E−13 | 0.367548918 | 0.415 | 0.285 | 4.59E−09 | A6 | Prrc2c |
| Cyp39a1 | 1.93E−13 | 0.292505843 | 0.305 | 0.183 | 4.80E−09 | A6 | Cyp39a1 |
| Pxk | 2.42E−13 | 0.256288767 | 0.303 | 0.177 | 6.01E−09 | A6 | Pxk |
| St3gal5 | 5.27E−13 | 0.296275564 | 0.706 | 0.63 | 1.31E−08 | A6 | St3gal5 |
| Lrrfip2 | 5.33E−13 | 0.352184936 | 0.384 | 0.258 | 1.32E−08 | A6 | Lrrfip2 |
| Mbnl1 | 6.12E−13 | 0.362674779 | 0.492 | 0.386 | 1.52E−08 | A6 | Mbnl1 |
| Fus | 1.20E−12 | 0.334188564 | 0.394 | 0.277 | 2.98E−08 | A6 | Fus |
| Pabpc1 | 1.23E−12 | 0.284772489 | 0.697 | 0.662 | 3.05E−08 | A6 | Pabpc1 |
| Arhgef12 | 1.39E−12 | 0.426872316 | 0.578 | 0.523 | 3.46E−08 | A6 | Arhgef12 |
| Stom | 1.51E−12 | 0.27534126 | 0.329 | 0.208 | 3.75E−08 | A6 | Stom |
| Srsf11 | 1.96E−12 | 0.380589282 | 0.418 | 0.299 | 4.86E−08 | A6 | Srsf11 |
| Nars | 2.53E−12 | 0.33663939 | 0.606 | 0.56 | 6.27E−08 | A6 | Nars |
| Ptpn12 | 4.40E−12 | 0.297316891 | 0.263 | 0.15 | 1.09E−07 | A6 | Ptpn12 |
| Palld | 5.39E−12 | 0.29349618 | 0.353 | 0.232 | 1.34E−07 | A6 | Palld |
| Il4ra | 8.30E−12 | 0.257153801 | 0.248 | 0.137 | 2.06E−07 | A6 | Il4ra |
| Ankrd11 | 1.52E−11 | 0.378964388 | 0.389 | 0.269 | 3.77E−07 | A6 | Ankrd11 |
| Dst | 1.78E−11 | 0.373962562 | 0.43 | 0.318 | 4.43E−07 | A6 | Dst |
| Mki67ip | 2.73E−11 | 0.298808984 | 0.344 | 0.227 | 6.77E−07 | A6 | Mki67ip |
| Cald1 | 4.43E−11 | 0.283138123 | 0.766 | 0.799 | 1.10E−06 | A6 | Cald1 |
| Phlda1 | 5.66E−11 | 0.451479481 | 0.494 | 0.445 | 1.41E−06 | A6 | Phlda1 |
| Ifrd1 | 6.04E−11 | 0.44712839 | 0.365 | 0.25 | 1.50E−06 | A6 | Ifrd1 |
| Pacsin2 | 6.38E−11 | 0.353993311 | 0.37 | 0.261 | 1.58E−06 | A6 | Pacsin2 |
| Fkbp5 | 6.69E−11 | 0.307808382 | 0.415 | 0.29 | 1.66E−06 | A6 | Fkbp5 |
| Smg1 | 7.01E−11 | 0.278924303 | 0.265 | 0.159 | 1.74E−06 | A6 | Smg1 |
| Tns1 | 7.04E−11 | 0.32885739 | 0.403 | 0.299 | 1.75E−06 | A6 | Tns1 |
| Gjb2 | 8.99E−11 | 0.324993861 | 0.678 | 0.703 | 2.23E−06 | A6 | Gjb2 |
| Mak16 | 1.07E−10 | 0.271983271 | 0.267 | 0.159 | 2.65E−06 | A6 | Mak16 |
| Map3k5 | 2.49E−10 | 0.27940085 | 0.346 | 0.234 | 6.19E−06 | A6 | Map3k5 |
| Chd1 | 2.74E−10 | 0.281548083 | 0.377 | 0.27 | 6.80E−06 | A6 | Chd1 |
| Kdelr2 | 3.61E−10 | 0.359811942 | 0.484 | 0.425 | 8.97E−06 | A6 | Kdelr2 |
| Cast | 3.73E−10 | 0.343500313 | 0.399 | 0.294 | 9.27E−06 | A6 | Cast |
| Smap2 | 4.35E−10 | 0.319039862 | 0.363 | 0.25 | 1.08E−05 | A6 | Smap2 |
| Purb | 4.61E−10 | 0.29384642 | 0.341 | 0.236 | 1.14E−05 | A6 | Purb |
| Man2a1 | 5.79E−10 | 0.274405257 | 0.671 | 0.689 | 1.44E−05 | A6 | Man2a1 |
| Wdr26 | 5.81E−10 | 0.346830997 | 0.549 | 0.49 | 1.44E−05 | A6 | Wdr26 |
| Sptbn1 | 7.00E−10 | 0.332175015 | 0.439 | 0.352 | 1.74E−05 | A6 | Sptbn1 |
| Coro1c | 1.40E−09 | 0.275517721 | 0.208 | 0.115 | 3.47E−05 | A6 | Coro1c |
| Sfpq | 1.53E−09 | 0.310914261 | 0.406 | 0.305 | 3.80E−05 | A6 | Sfpq |
| Eif4g1 | 1.58E−09 | 0.274946516 | 0.723 | 0.737 | 3.92E−05 | A6 | Eif4g1 |
| Gm5424 | 1.92E−09 | 0.319966931 | 0.411 | 0.312 | 4.78E−05 | A6 | Gm5424 |
| AA474408 | 2.16E−09 | 0.267890734 | 0.274 | 0.172 | 5.37E−05 | A6 | AA474408 |
| Ddx6 | 2.21E−09 | 0.368176844 | 0.527 | 0.459 | 5.49E−05 | A6 | Ddx6 |
| Rbm25 | 2.60E−09 | 0.371600026 | 0.461 | 0.363 | 6.46E−05 | A6 | Rbm25 |
| Akap13 | 2.60E−09 | 0.408483485 | 0.406 | 0.304 | 6.47E−05 | A6 | Akap13 |
| Metap2 | 2.78E−09 | 0.304477182 | 0.453 | 0.371 | 6.89E−05 | A6 | Metap2 |
| Chmp4b | 3.12E−09 | 0.282546158 | 0.442 | 0.353 | 7.76E−05 | A6 | Chmp4b |
| Atf4 | 3.46E−09 | 0.277274796 | 0.58 | 0.507 | 8.59E−05 | A6 | Atf4 |
| Gsr | 4.92E−09 | 0.367675526 | 0.453 | 0.367 | 0.0001221 | A6 | Gsr |
| Slc4a4 | 5.68E−09 | 0.297606513 | 0.513 | 0.441 | 0.000140988 | A6 | Slc4a4 |
| Uhrf1bp1l | 6.07E−09 | 0.33260243 | 0.399 | 0.307 | 0.000150772 | A6 | Uhrf1bp1l |
| Gsk3b | 6.43E−09 | 0.329286038 | 0.384 | 0.29 | 0.000159674 | A6 | Gsk3b |
| Atp2a2 | 6.85E−09 | 0.309886544 | 0.616 | 0.603 | 0.000170151 | A6 | Atp2a2 |
| Reep3 | 7.41E−09 | 0.321347377 | 0.535 | 0.514 | 0.000183969 | A6 | Reep3 |
| Tpr | 9.24E−09 | 0.356753149 | 0.501 | 0.429 | 0.000229341 | A6 | Tpr |
| Rnd3 | 9.97E−09 | 0.267699227 | 0.284 | 0.187 | 0.000247483 | A6 | Rnd3 |
| Huwe1 | 1.28E−08 | 0.285654225 | 0.492 | 0.406 | 0.000318913 | A6 | Huwe1 |
| Eif4a1 | 1.29E−08 | 0.275359204 | 0.418 | 0.327 | 0.000319298 | A6 | Eif4a1 |
| Sf1 | 1.36E−08 | 0.257203905 | 0.351 | 0.248 | 0.000337793 | A6 | Sf1 |
| Hmgcr | 1.74E−08 | 0.346160341 | 0.425 | 0.342 | 0.000432829 | A6 | Hmgcr |
| Grn | 2.07E−08 | 0.295536999 | 0.582 | 0.55 | 0.000513486 | A6 | Grn |
| Egr1 | 2.14E−08 | 0.370050393 | 0.589 | 0.54 | 0.0005325 | A6 | Egr1 |
| Scaf11 | 2.17E−08 | 0.309964438 | 0.449 | 0.377 | 0.000539745 | A6 | Scaf11 |
| Bptf | 2.28E−08 | 0.275194478 | 0.332 | 0.232 | 0.000565519 | A6 | Bptf |
| Tmed5 | 2.47E−08 | 0.272917209 | 0.594 | 0.583 | 0.000612693 | A6 | Tmed5 |
| Sbno1 | 2.47E−08 | 0.299396593 | 0.394 | 0.306 | 0.000614253 | A6 | Sbno1 |
| Dhx9 | 3.39E−08 | 0.285323361 | 0.427 | 0.339 | 0.000842741 | A6 | Dhx9 |
| Slc40a1 | 4.45E−08 | 0.412382652 | 0.456 | 0.378 | 0.001105109 | A6 | Slc40a1 |
| Lpgat1 | 4.50E−08 | 0.342974763 | 0.618 | 0.636 | 0.001117229 | A6 | Lpgat1 |
| Gm26917 | 7.14E−08 | 0.299154579 | 0.382 | 0.289 | 0.001772559 | A6 | Gm26917 |
| Birc6 | 8.11E−08 | 0.277616901 | 0.446 | 0.354 | 0.002013406 | A6 | Birc6 |
| Rsl1d1 | 1.02E−07 | 0.303430543 | 0.47 | 0.411 | 0.002521012 | A6 | Rsl1d1 |
| Hnrnpu | 1.09E−07 | 0.28326413 | 0.625 | 0.639 | 0.002697817 | A6 | Hnrnpu |
| Anxa5 | 1.15E−07 | 0.305834928 | 0.391 | 0.307 | 0.002851117 | A6 | Anxa5 |
| Kif21a | 1.26E−07 | 0.313315683 | 0.461 | 0.385 | 0.003128928 | A6 | Kif21a |
| Gtf2a2 | 1.39E−07 | 0.251999937 | 0.379 | 0.292 | 0.003462804 | A6 | Gtf2a2 |
| Akap11 | 1.43E−07 | 0.270661136 | 0.351 | 0.259 | 0.00355934 | A6 | Akap11 |
| Clic4 | 1.72E−07 | 0.264181697 | 0.368 | 0.283 | 0.004262249 | A6 | Clic4 |
| Mapkapk2 | 2.32E−07 | 0.294517429 | 0.396 | 0.311 | 0.005755355 | A6 | Mapkapk2 |
| Atp13a3 | 2.93E−07 | 0.292349764 | 0.532 | 0.505 | 0.007280754 | A6 | Atp13a3 |
| Net1 | 3.22E−07 | 0.306659682 | 0.408 | 0.332 | 0.007991182 | A6 | Net1 |
| Zbtb20 | 3.23E−07 | 0.327627617 | 0.566 | 0.539 | 0.008021841 | A6 | Zbtb20 |
| Rlim | 9.49E−07 | 0.252627877 | 0.305 | 0.22 | 0.02357139 | A6 | Rlim |
| Actr3 | 1.12E−06 | 0.25984234 | 0.36 | 0.28 | 0.027851102 | A6 | Actr3 |
| Tnks2 | 1.30E−06 | 0.254002601 | 0.358 | 0.281 | 0.032193406 | A6 | Tnks2 |
| BC005537 | 1.32E−06 | 0.289191965 | 0.544 | 0.509 | 0.03283521 | A6 | BC005537 |
| Chuk | 1.38E−06 | 0.310357965 | 0.523 | 0.489 | 0.034209316 | A6 | Chuk |
| Phf20l1 | 1.39E−06 | 0.265257027 | 0.372 | 0.292 | 0.03450551 | A6 | Phf20l1 |
| Mdm2 | 1.67E−06 | 0.259049205 | 0.305 | 0.222 | 0.041535876 | A6 | Mdm2 |
| Brd2 | 2.12E−06 | 0.433254813 | 0.542 | 0.504 | 0.052591494 | A6 | Brd2 |
| Atp2b1 | 2.38E−06 | 0.280653791 | 0.334 | 0.254 | 0.05897613 | A6 | Atp2b1 |
| Mpeg1 | 4.38E−06 | 0.352914126 | 0.263 | 0.183 | 0.108746855 | A6 | Mpeg1 |
| 4932438A13Rik | 7.01E−06 | 0.266199526 | 0.329 | 0.251 | 0.174064494 | A6 | 4932438A13Rik |
| Dnajc5 | 7.09E−06 | 0.250353453 | 0.372 | 0.308 | 0.176120819 | A6 | Dnajc5 |
| Mgea5 | 7.20E−06 | 0.275686568 | 0.413 | 0.342 | 0.178891521 | A6 | Mgea5 |
| Dync1h1 | 9.59E−06 | 0.282699593 | 0.379 | 0.306 | 0.238121164 | A6 | Dync1h1 |
| Srsf5 | 1.19E−05 | 0.291409476 | 0.387 | 0.322 | 0.296477533 | A6 | Srsf5 |
| Srrm2 | 1.36E−05 | 0.260727453 | 0.456 | 0.397 | 0.338662468 | A6 | Srrm2 |
| Ddx3y | 1.38E−05 | 0.288586131 | 0.47 | 0.429 | 0.342849995 | A6 | Ddx3y |
| Kdm5a | 1.60E−05 | 0.304429913 | 0.308 | 0.231 | 0.396217599 | A6 | Kdm5a |
| Xrn2 | 1.82E−05 | 0.259675732 | 0.387 | 0.321 | 0.450954074 | A6 | Xrn2 |
| Dnaja2 | 2.12E−05 | 0.279328244 | 0.516 | 0.493 | 0.52548196 | A6 | Dnaja2 |
| Eif4ebp1 | 3.29E−05 | 0.259521234 | 0.387 | 0.328 | 0.817137366 | A6 | Eif4ebp1 |
| Zfand5 | 3.57E−05 | 0.275089578 | 0.401 | 0.339 | 0.887556739 | A6 | Zfand5 |
| Plk3 | 3.83E−05 | 0.283547851 | 0.413 | 0.346 | 0.951959871 | A6 | Plk3 |
| Macf1 | 5.05E−05 | 0.301160502 | 0.377 | 0.313 | 1 | A6 | Macf1 |
| Pi4k2a | 6.34E−05 | 0.305951761 | 0.425 | 0.391 | 1 | A6 | Pi4k2a |
| Lrrc59 | 6.81E−05 | 0.259229523 | 0.422 | 0.382 | 1 | A6 | Lrrc59 |
| Eprs | 7.81E−05 | 0.269947199 | 0.566 | 0.572 | 1 | A6 | Eprs |
| Diap1 | 9.69E−05 | 0.270444484 | 0.382 | 0.318 | 1 | A6 | Diap1 |
| Ctsb | 0.000138715 | 0.323201059 | 0.804 | 0.852 | 1 | A6 | Ctsb |
| Tab2 | 0.000169421 | 0.286847887 | 0.425 | 0.382 | 1 | A6 | Tab2 |
| Rnf169 | 0.000193224 | 0.282809956 | 0.315 | 0.253 | 1 | A6 | Rnf169 |
| Rap1b | 0.000204185 | 0.259227104 | 0.406 | 0.355 | 1 | A6 | Rap1b |
| Abcc2 | 0.000314242 | 0.250146223 | 0.632 | 0.663 | 1 | A6 | Abcc2 |
| Eif4b | 0.000334975 | 0.262813454 | 0.542 | 0.538 | 1 | A6 | Eif4b |
| Kif1b | 0.00036934 | 0.299926146 | 0.527 | 0.513 | 1 | A6 | Kif1b |
| Zfp106 | 0.000422039 | 0.266717149 | 0.391 | 0.337 | 1 | A6 | Zfp106 |
| Hectd1 | 0.00059151 | 0.251508749 | 0.547 | 0.536 | 1 | A6 | Hectd1 |
| Nrp1 | 0.000622747 | 0.252678382 | 0.391 | 0.344 | 1 | A6 | Nrp1 |
| Impad1 | 0.001316802 | 0.254658012 | 0.391 | 0.355 | 1 | A6 | Impad1 |
| Atp1a1 | 0.001836487 | 0.262331948 | 0.53 | 0.561 | 1 | A6 | Atp1a1 |
| Riok3 | 0.004729208 | 0.256616769 | 0.43 | 0.412 | 1 | A6 | Riok3 |
| Trip11 | 0.005826406 | 0.273288464 | 0.341 | 0.302 | 1 | A6 | Trip11 |
| Tln1 | 0.008537106 | 0.278523779 | 0.31 | 0.269 | 1 | A6 | Tln1 |
| Grip2 | 0 | 0.877662322 | 0.475 | 0.002 | 0 | A24 | Grip2 |
| Sephs21 | 8.05E−198 | 0.61331088 | 0.949 | 0.833 | 2.00E−193 | A24 | Sephs2 |
| Gm269241 | 1.37E−184 | 0.628825902 | 1 | 1 | 3.39E−180 | A24 | Gm26924 |
| Sepp1 | 4.76E−166 | 0.318542032 | 1 | 1 | 1.18E−161 | A24 | Sepp1 |
| Hsp90b11 | 9.73E−149 | 0.509570205 | 0.954 | 0.95 | 2.42E−144 | A24 | Hsp90b1 |
| Hspa5 | 1.96E−146 | 0.449684973 | 0.974 | 0.965 | 4.87E−142 | A24 | Hspa5 |
| Pdia31 | 2.07E−144 | 0.546318119 | 0.895 | 0.824 | 5.14E−140 | A24 | Pdia3 |
| Mup71 | 6.46E−140 | 0.375529532 | 0.985 | 0.969 | 1.60E−135 | A24 | Mup7 |
| Gm155641 | 1.14E−129 | 1.14097252 | 0.982 | 0.976 | 2.83E−125 | A24 | Gm15564 |
| Calr1 | 1.88E−129 | 0.45086474 | 0.952 | 0.906 | 4.67E−125 | A24 | Calr |
| Fgb | 5.44E−127 | 0.367238117 | 1 | 0.999 | 1.35E−122 | A24 | Fgb |
| Mup121 | 6.26E−126 | 0.68150125 | 0.799 | 0.668 | 1.56E−121 | A24 | Mup12 |
| Arrdc3 | 8.88E−116 | 0.54775482 | 0.52 | 0.24 | 2.21E−111 | A24 | Arrdc3 |
| Hyou1 | 1.13E−113 | 0.638165516 | 0.717 | 0.515 | 2.82E−109 | A24 | Hyou1 |
| Lars21 | 3.10E−111 | 1.041057155 | 0.998 | 0.997 | 7.70E−107 | A24 | Lars2 |
| Sc5d1 | 6.21E−89 | 0.412371965 | 0.879 | 0.816 | 1.54E−84 | A24 | Sc5d |
| C8b1 | 1.11E−85 | 0.384982182 | 0.898 | 0.879 | 2.75E−81 | A24 | C8b |
| Fgg | 1.64E−84 | 0.272181625 | 0.999 | 0.996 | 4.06E−80 | A24 | Fgg |
| Fga | 9.15E−71 | 0.254248466 | 0.998 | 0.997 | 2.27E−66 | A24 | Fga |
| Hsd3b51 | 9.32E−70 | 0.441836795 | 0.638 | 0.418 | 2.31E−65 | A24 | Hsd3b5 |
| Dnajc3 | 6.35E−69 | 0.392858395 | 0.85 | 0.813 | 1.58E−64 | A24 | Dnajc3 |
| Pgrmc1 | 2.43E−68 | 0.289998299 | 0.953 | 0.949 | 6.04E−64 | A24 | Pgrmc1 |
| Hdlbp | 3.97E−68 | 0.408547423 | 0.853 | 0.783 | 9.86E−64 | A24 | Hdlbp |
| Ssr3 | 2.31E−65 | 0.403074533 | 0.765 | 0.675 | 5.74E−61 | A24 | Ssr3 |
| Pdia41 | 5.72E−65 | 0.459428501 | 0.733 | 0.644 | 1.42E−60 | A24 | Pdia4 |
| Creld2 | 5.19E−64 | 0.485966761 | 0.583 | 0.403 | 1.29E−59 | A24 | Creld2 |
| Glud1 | 5.85E−61 | 0.27854336 | 0.958 | 0.939 | 1.45E−56 | A24 | Glud1 |
| Pdcd41 | 4.60E−59 | 0.456773789 | 0.677 | 0.548 | 1.14E−54 | A24 | Pdcd4 |
| Eef1a1 | 3.88E−58 | 0.322152231 | 0.919 | 0.882 | 9.63E−54 | A24 | Eef1a1 |
| C91 | 2.86E−57 | 0.289294558 | 0.952 | 0.934 | 7.11E−53 | A24 | C9 |
| Elovl2 | 1.70E−53 | 0.36774725 | 0.826 | 0.762 | 4.21E−49 | A24 | Elovl2 |
| Tram1 | 9.96E−51 | 0.35532977 | 0.7 | 0.585 | 2.47E−46 | A24 | Tram1 |
| Pdia61 | 2.91E−50 | 0.368367226 | 0.779 | 0.718 | 7.23E−46 | A24 | Pdia6 |
| Serp1 | 9.85E−50 | 0.365823084 | 0.809 | 0.752 | 2.45E−45 | A24 | Serp1 |
| Calm1 | 1.21E−48 | 0.348585861 | 0.794 | 0.77 | 3.01E−44 | A24 | Calm1 |
| Manf | 4.99E−48 | 0.400885006 | 0.632 | 0.507 | 1.24E−43 | A24 | Manf |
| Insig1 | 7.91E−48 | 0.40460413 | 0.626 | 0.496 | 1.96E−43 | A24 | Insig1 |
| Hao11 | 8.37E−47 | 0.329177975 | 0.83 | 0.789 | 2.08E−42 | A24 | Hao1 |
| Nudt71 | 2.49E−46 | 0.282119575 | 0.884 | 0.799 | 6.20E−42 | A24 | Nudt7 |
| AI1823711 | 1.39E−45 | 0.35855032 | 0.747 | 0.686 | 3.45E−41 | A24 | AI182371 |
| Iigp11 | 2.89E−44 | 0.297964443 | 0.832 | 0.808 | 7.17E−40 | A24 | Iigp1 |
| Egfr | 7.06E−43 | 0.295191072 | 0.882 | 0.861 | 1.75E−38 | A24 | Egfr |
| Aox31 | 3.70E−41 | 0.31657291 | 0.916 | 0.894 | 9.20E−37 | A24 | Aox3 |
| Rpn1 | 6.65E−39 | 0.399582378 | 0.605 | 0.497 | 1.65E−34 | A24 | Rpn1 |
| Tmem30a | 1.31E−38 | 0.309658036 | 0.766 | 0.719 | 3.25E−34 | A24 | Tmem30a |
| n-R5-8s1 | 4.55E−38 | 0.526714633 | 0.522 | 0.323 | 1.13E−33 | A24 | n-R5-8s1 |
| Atxn1 | 7.15E−37 | 0.303135424 | 0.311 | 0.17 | 1.77E−32 | A24 | Atxn1 |
| Nr1d2 | 9.32E−37 | 0.347216847 | 0.442 | 0.295 | 2.31E−32 | A24 | Nr1d2 |
| Hspa13 | 2.28E−36 | 0.308966854 | 0.404 | 0.257 | 5.65E−32 | A24 | Hspa13 |
| Cyp2c701 | 1.85E−35 | 0.259617473 | 0.89 | 0.868 | 4.60E−31 | A24 | Cyp2c70 |
| Gm24601 | 2.21E−34 | 0.54562503 | 0.404 | 0.241 | 5.49E−30 | A24 | Gm24601 |
| Spcs3 | 8.19E−34 | 0.329508814 | 0.483 | 0.358 | 2.03E−29 | A24 | Spcs3 |
| Gm54241 | 1.03E−33 | 0.31004204 | 0.445 | 0.302 | 2.57E−29 | A24 | Gm5424 |
| Tmed7 | 5.31E−33 | 0.326955086 | 0.563 | 0.446 | 1.32E−28 | A24 | Tmed7 |
| Adh41 | 3.43E−32 | 0.338184013 | 0.667 | 0.56 | 8.51E−28 | A24 | Adh4 |
| Eif4g21 | 2.08E−31 | 0.258833943 | 0.8 | 0.771 | 5.18E−27 | A24 | Eif4g2 |
| Prkd31 | 3.77E−31 | 0.336938928 | 0.631 | 0.543 | 9.36E−27 | A24 | Prkd3 |
| Myh91 | 3.86E−29 | 0.329906697 | 0.595 | 0.479 | 9.59E−25 | A24 | Myh9 |
| Akr1c141 | 5.50E−29 | 0.284278295 | 0.74 | 0.701 | 1.37E−24 | A24 | Akr1c14 |
| Hsd17b21 | 9.92E−29 | 0.264739028 | 0.758 | 0.706 | 2.46E−24 | A24 | Hsd17b2 |
| Ostc | 1.19E−28 | 0.289134091 | 0.514 | 0.385 | 2.96E−24 | A24 | Ostc |
| Sdf2l1 | 1.69E−28 | 0.260614388 | 0.412 | 0.278 | 4.19E−24 | A24 | Sdf2l1 |
| Gm242451 | 2.43E−28 | 0.479708393 | 0.369 | 0.241 | 6.03E−24 | A24 | Gm24245 |
| Rdx1 | 3.10E−28 | 0.295224413 | 0.729 | 0.69 | 7.70E−24 | A24 | Rdx |
| Uso1 | 6.01E−28 | 0.310192088 | 0.597 | 0.519 | 1.49E−23 | A24 | Uso1 |
| Pnpla71 | 2.02E−26 | 0.295524363 | 0.678 | 0.604 | 5.02E−22 | A24 | Pnpla7 |
| Atp8b1 | 3.46E−26 | 0.294111116 | 0.391 | 0.266 | 8.59E−22 | A24 | Atp8b1 |
| AA4744081 | 2.18E−25 | 0.390602267 | 0.278 | 0.165 | 5.41E−21 | A24 | AA474408 |
| Stard4 | 1.38E−24 | 0.309852489 | 0.559 | 0.461 | 3.43E−20 | A24 | Stard4 |
| Cltc1 | 6.57E−24 | 0.285288939 | 0.61 | 0.516 | 1.63E−19 | A24 | Cltc |
| Usp4 | 1.52E−23 | 0.272747149 | 0.476 | 0.361 | 3.78E−19 | A24 | Usp4 |
| Cmah1 | 3.19E−23 | 0.295776656 | 0.607 | 0.532 | 7.93E−19 | A24 | Cmah |
| Fndc3b1 | 1.08E−22 | 0.250245162 | 0.291 | 0.178 | 2.67E−18 | A24 | Fndc3b |
| Gm269171 | 5.09E−22 | 0.581927547 | 0.391 | 0.282 | 1.26E−17 | A24 | Gm26917 |
| Sec61b1 | 5.10E−22 | 0.264836278 | 0.549 | 0.461 | 1.27E−17 | A24 | Sec61b |
| Acly | 7.83E−22 | 0.300495396 | 0.597 | 0.503 | 1.94E−17 | A24 | Acly |
| Srebf1 | 2.75E−21 | 0.287445553 | 0.439 | 0.334 | 6.83E−17 | A24 | Srebf1 |
| Dnajb11 | 6.25E−21 | 0.299844177 | 0.557 | 0.485 | 1.55E−16 | A24 | Dnajb11 |
| Dnajc12 | 8.12E−21 | 0.256463813 | 0.275 | 0.173 | 2.02E−16 | A24 | Dnajc12 |
| Usp9x | 2.28E−19 | 0.28533222 | 0.502 | 0.411 | 5.66E−15 | A24 | Usp9x |
| Rab1 | 1.00E−18 | 0.258167749 | 0.588 | 0.527 | 2.49E−14 | A24 | Rab1 |
| Arcn1 | 2.36E−18 | 0.259397317 | 0.53 | 0.447 | 5.86E−14 | A24 | Arcn1 |
| Etnk1 | 9.72E−18 | 0.276144874 | 0.49 | 0.41 | 2.41E−13 | A24 | Etnk1 |
| Ctnnb1 | 1.62E−17 | 0.250000942 | 0.566 | 0.498 | 4.03E−13 | A24 | Ctnnb1 |
| Nedd4 | 3.77E−17 | 0.255257894 | 0.647 | 0.615 | 9.36E−13 | A24 | Nedd4 |
| Magt1 | 2.47E−16 | 0.252990053 | 0.52 | 0.464 | 6.14E−12 | A24 | Magt1 |
| Nab1 | 2.71E−16 | 0.253358398 | 0.491 | 0.408 | 6.73E−12 | A24 | Nab1 |
| Ptprf1 | 3.30E−16 | 0.251224059 | 0.588 | 0.532 | 8.21E−12 | A24 | Ptprf |
| Nars1 | 6.06E−14 | 0.265707299 | 0.593 | 0.558 | 1.50E−09 | A24 | Nars |
| Cyp1a21 | 2.13E−06 | 0.25313456 | 0.699 | 0.709 | 0.052836876 | A24 | Cyp1a2 |
| Cyp4a142 | 0 | 1.487902827 | 0.991 | 0.45 | 0 | A48 | Cyp4a14 |
| Cyp4a102 | 0 | 1.156719472 | 0.991 | 0.574 | 0 | A48 | Cyp4a10 |
| Hmgcs21 | 0 | 0.819620651 | 1 | 0.972 | 0 | A48 | Hmgcs2 |
| Chchd102 | 0 | 0.813974324 | 0.995 | 0.931 | 0 | A48 | Chchd10 |
| Angptl3 | 0 | 0.770515169 | 0.995 | 0.945 | 0 | A48 | Angptl3 |
| Mat1a2 | 0 | 0.658541776 | 1 | 0.976 | 0 | A48 | Mat1a |
| Apoe2 | 0 | 0.610213342 | 1 | 1 | 0 | A48 | Apoe |
| Ces1c1 | 0 | 0.586094458 | 0.999 | 0.986 | 0 | A48 | Ces1c |
| Apoh1 | 1.40E−296 | 0.535860118 | 0.998 | 0.982 | 3.47E−292 | A48 | Apoh |
| Ttc361 | 3.49E−294 | 0.614534948 | 0.996 | 0.937 | 8.67E−290 | A48 | Ttc36 |
| Retsat2 | 8.18E−294 | 0.873440086 | 0.988 | 0.769 | 2.03E−289 | A48 | Retsat |
| Ugt2b52 | 3.37E−269 | 0.610774194 | 0.996 | 0.939 | 8.37E−265 | A48 | Ugt2b5 |
| Apoc2 | 4.66E−262 | 0.814207924 | 0.98 | 0.82 | 1.16E−257 | A48 | Apoc2 |
| Rdh71 | 1.24E−251 | 0.557485283 | 0.996 | 0.958 | 3.07E−247 | A48 | Rdh7 |
| Rbp4 | 6.09E−248 | 0.513931452 | 1 | 0.996 | 1.51E−243 | A48 | Rbp4 |
| Abcd2 | 3.35E−245 | 0.290615784 | 0.473 | 0.091 | 8.32E−241 | A48 | Abcd2 |
| Dbp | 1.52E−225 | 0.292912608 | 0.52 | 0.117 | 3.78E−221 | A48 | Dbp |
| Atp5h2 | 1.28E−220 | 0.731224311 | 0.933 | 0.613 | 3.17E−216 | A48 | Atp5h |
| Decr12 | 3.47E−218 | 0.594105071 | 0.96 | 0.679 | 8.62E−214 | A48 | Decr1 |
| Slco1a4 | 5.77E−218 | 0.546576628 | 0.77 | 0.284 | 1.43E−213 | A48 | Slco1a4 |
| Herpud11 | 9.23E−217 | 0.596486042 | 0.985 | 0.815 | 2.29E−212 | A48 | Herpud1 |
| Cyp3a251 | 1.10E−215 | 0.506946657 | 0.995 | 0.884 | 2.72E−211 | A48 | Cyp3a25 |
| Nr1i32 | 1.91E−205 | 0.573928423 | 0.814 | 0.331 | 4.75E−201 | A48 | Nr1i3 |
| Agt1 | 1.51E−199 | 0.533913518 | 0.981 | 0.837 | 3.75E−195 | A48 | Agt |
| Ephx21 | 5.54E−196 | 0.486375274 | 0.993 | 0.905 | 1.38E−191 | A48 | Ephx2 |
| Reep61 | 1.39E−194 | 0.590173225 | 0.967 | 0.81 | 3.44E−190 | A48 | Reep6 |
| F2 | 3.96E−192 | 0.376680538 | 0.999 | 0.974 | 9.83E−188 | A48 | F2 |
| Mfsd2a2 | 1.42E−188 | 0.582749125 | 0.731 | 0.276 | 3.53E−184 | A48 | Mfsd2a |
| Ahcy1 | 1.94E−186 | 0.473933613 | 0.988 | 0.858 | 4.82E−182 | A48 | Ahcy |
| Fth1 | 1.04E−184 | 0.434153916 | 0.996 | 0.978 | 2.58E−180 | A48 | Fth1 |
| Apoa51 | 2.80E−184 | 0.436646528 | 0.998 | 0.957 | 6.96E−180 | A48 | Apoa5 |
| Snord1181 | 5.48E−184 | 0.528640807 | 0.766 | 0.303 | 1.36E−179 | A48 | Snord118 |
| Hmgcl | 2.36E−183 | 0.619035847 | 0.956 | 0.712 | 5.87E−179 | A48 | Hmgcl |
| Oaf1 | 2.57E−183 | 0.561886634 | 0.969 | 0.776 | 6.38E−179 | A48 | Oaf |
| Hdac111 | 8.97E−179 | 0.3963119 | 0.713 | 0.267 | 2.23E−174 | A48 | Hdac11 |
| Ttr1 | 1.32E−175 | 0.420507995 | 1 | 0.999 | 3.28E−171 | A48 | Ttr |
| Rpl38 | 5.45E−174 | 0.345542874 | 0.596 | 0.193 | 1.35E−169 | A48 | Rpl38 |
| Ttpa | 2.01E−169 | 0.475726856 | 0.991 | 0.885 | 4.99E−165 | A48 | Ttpa |
| Rgs161 | 2.49E−168 | 0.380333199 | 0.601 | 0.193 | 6.19E−164 | A48 | Rgs16 |
| Ech11 | 2.07E−167 | 0.525118681 | 0.98 | 0.808 | 5.15E−163 | A48 | Ech1 |
| Ces1b | 2.28E−167 | 0.530778378 | 0.838 | 0.434 | 5.66E−163 | A48 | Ces1b |
| Gm6484 | 5.35E−166 | 0.375910184 | 0.627 | 0.213 | 1.33E−161 | A48 | Gm6484 |
| Khk2 | 8.30E−162 | 0.533840979 | 0.982 | 0.866 | 2.06E−157 | A48 | Khk |
| Cyp2a12 | 1.12E−161 | 0.520687928 | 0.981 | 0.872 | 2.78E−157 | A48 | Cyp2a12 |
| Lims2 | 2.85E−161 | 0.393099584 | 0.679 | 0.266 | 7.08E−157 | A48 | Lims2 |
| Prodh2 | 5.22E−160 | 0.517905408 | 0.918 | 0.61 | 1.30E−155 | A48 | Prodh2 |
| Bnip32 | 5.10E−158 | 0.526170876 | 0.876 | 0.517 | 1.27E−153 | A48 | Bnip3 |
| Aldob | 9.73E−158 | 0.375754846 | 1 | 0.996 | 2.42E−153 | A48 | Aldob |
| Slc25a471 | 1.66E−156 | 0.524948065 | 0.992 | 0.913 | 4.13E−152 | A48 | Slc25a47 |
| Apoc42 | 3.51E−154 | 0.50531584 | 0.998 | 0.974 | 8.72E−150 | A48 | Apoc4 |
| Lcat1 | 5.07E−154 | 0.47331748 | 0.973 | 0.796 | 1.26E−149 | A48 | Lcat |
| Amy11 | 3.72E−153 | 0.494980149 | 0.943 | 0.644 | 9.23E−149 | A48 | Amy1 |
| Tef | 2.06E−152 | 0.316225396 | 0.651 | 0.241 | 5.12E−148 | A48 | Tef |
| Hrg1 | 3.76E−150 | 0.435967486 | 0.987 | 0.867 | 9.34E−146 | A48 | Hrg |
| Pabpc11 | 3.95E−150 | 0.511620216 | 0.912 | 0.64 | 9.80E−146 | A48 | Pabpc1 |
| Serpina1d1 | 1.77E−149 | 0.356258396 | 0.997 | 0.974 | 4.39E−145 | A48 | Serpina1d |
| Cdo11 | 4.20E−148 | 0.430080331 | 0.997 | 0.957 | 1.04E−143 | A48 | Cdo1 |
| Mlycd1 | 1.59E−147 | 0.463720831 | 0.893 | 0.506 | 3.94E−143 | A48 | Mlycd |
| Slc27a51 | 1.82E−147 | 0.410011543 | 0.987 | 0.897 | 4.52E−143 | A48 | Slc27a5 |
| Gstt3 | 2.24E−147 | 0.38336004 | 0.737 | 0.302 | 5.56E−143 | A48 | Gstt3 |
| Echs11 | 3.16E−147 | 0.492572199 | 0.971 | 0.812 | 7.85E−143 | A48 | Echs1 |
| Serping11 | 2.48E−145 | 0.443468356 | 0.987 | 0.893 | 6.16E−141 | A48 | Serping1 |
| Ndrg21 | 2.32E−144 | 0.349757727 | 1 | 0.945 | 5.75E−140 | A48 | Ndrg2 |
| Vtn2 | 3.16E−144 | 0.363779131 | 1 | 0.986 | 7.86E−140 | A48 | Vtn |
| Pnpla2 | 7.80E−143 | 0.345688119 | 0.687 | 0.28 | 1.94E−138 | A48 | Pnpla2 |
| Ube2r21 | 6.65E−142 | 0.467949325 | 0.884 | 0.55 | 1.65E−137 | A48 | Ube2r2 |
| Apoc12 | 1.83E−141 | 0.363822891 | 1 | 0.994 | 4.54E−137 | A48 | Apoc1 |
| Cox6a12 | 5.35E−141 | 0.447227091 | 0.987 | 0.848 | 1.33E−136 | A48 | Cox6a1 |
| Ahsg1 | 4.13E−140 | 0.250603949 | 1 | 0.999 | 1.02E−135 | A48 | Ahsg |
| Apoc32 | 4.73E−140 | 0.363390224 | 1 | 0.982 | 1.17E−135 | A48 | Apoc3 |
| Abcb4 | 7.97E−139 | 0.470174782 | 0.956 | 0.697 | 1.98E−134 | A48 | Abcb4 |
| Slc25a101 | 2.28E−137 | 0.453932455 | 0.877 | 0.535 | 5.65E−133 | A48 | Slc25a10 |
| Scand1 | 3.35E−137 | 0.258140676 | 0.579 | 0.204 | 8.31E−133 | A48 | Scand1 |
| Cyp3a112 | 1.28E−136 | 0.49808554 | 1 | 0.997 | 3.19E−132 | A48 | Cyp3a11 |
| Slc10a11 | 2.15E−136 | 0.456196482 | 0.984 | 0.842 | 5.33E−132 | A48 | Slc10a1 |
| Cebpb2 | 1.30E−135 | 0.423057947 | 0.815 | 0.421 | 3.22E−131 | A48 | Cebpb |
| Esrra | 3.53E−134 | 0.251776143 | 0.546 | 0.19 | 8.77E−130 | A48 | Esrra |
| F101 | 6.00E−134 | 0.387936432 | 0.989 | 0.899 | 1.49E−129 | A48 | F10 |
| Creg1 | 1.75E−133 | 0.418044745 | 0.993 | 0.907 | 4.35E−129 | A48 | Creg1 |
| Atp5b | 4.40E−133 | 0.345066858 | 0.997 | 0.942 | 1.09E−128 | A48 | Atp5b |
| Fcgrt1 | 1.52E−131 | 0.431405292 | 0.91 | 0.585 | 3.78E−127 | A48 | Fcgrt |
| Syvn1 | 1.64E−130 | 0.286655562 | 0.603 | 0.228 | 4.06E−126 | A48 | Syvn1 |
| Slc25a20 | 1.74E−130 | 0.42083621 | 0.877 | 0.51 | 4.31E−126 | A48 | Slc25a20 |
| Rpl4 | 2.29E−130 | 0.374416508 | 0.987 | 0.914 | 5.69E−126 | A48 | Rpl4 |
| G0s21 | 7.23E−130 | 0.42316466 | 0.692 | 0.297 | 1.80E−125 | A48 | G0s2 |
| Gabarap1 | 4.55E−128 | 0.437625588 | 0.956 | 0.753 | 1.13E−123 | A48 | Gabarap |
| Gltscr2 | 9.76E−128 | 0.375879873 | 0.83 | 0.445 | 2.42E−123 | A48 | Gltscr2 |
| Aldh9a1 | 2.74E−127 | 0.414716257 | 0.964 | 0.776 | 6.79E−123 | A48 | Aldh9a1 |
| Ctsh | 1.74E−126 | 0.464904773 | 0.945 | 0.718 | 4.32E−122 | A48 | Ctsh |
| Cox8a2 | 2.32E−126 | 0.520209572 | 0.89 | 0.63 | 5.76E−122 | A48 | Cox8a |
| Tmbim61 | 6.05E−126 | 0.3566995 | 0.996 | 0.952 | 1.50E−121 | A48 | Tmbim6 |
| Gys21 | 6.92E−126 | 0.418222807 | 0.784 | 0.403 | 1.72E−121 | A48 | Gys2 |
| Etfb1 | 1.50E−125 | 0.354169101 | 0.996 | 0.93 | 3.73E−121 | A48 | Etfb |
| Hsd17b131 | 3.50E−125 | 0.642674937 | 0.934 | 0.739 | 8.68E−121 | A48 | Hsd17b13 |
| Decr2 | 3.28E−124 | 0.430802372 | 0.92 | 0.652 | 8.15E−120 | A48 | Decr2 |
| Etnppl2 | 6.28E−124 | 0.604543103 | 0.866 | 0.551 | 1.56E−119 | A48 | Etnppl |
| Tmbim4 | 1.38E−123 | 0.393095222 | 0.784 | 0.413 | 3.42E−119 | A48 | Tmbim4 |
| Vnn3 | 3.25E−123 | 0.305389222 | 0.684 | 0.291 | 8.07E−119 | A48 | Vnn3 |
| Slc40a11 | 2.35E−122 | 0.331978458 | 0.744 | 0.347 | 5.83E−118 | A48 | Slc40a1 |
| Fam195a | 1.14E−121 | 0.354389232 | 0.726 | 0.333 | 2.83E−117 | A48 | Fam195a |
| Sdhc | 2.25E−121 | 0.433373604 | 0.91 | 0.645 | 5.58E−117 | A48 | Sdhc |
| Bfar | 2.55E−121 | 0.301816412 | 0.742 | 0.331 | 6.32E−117 | A48 | Bfar |
| Serpinf1 | 3.44E−121 | 0.395394253 | 0.976 | 0.857 | 8.54E−117 | A48 | Serpinf1 |
| Atp5g31 | 6.91E−120 | 0.3996086 | 0.985 | 0.882 | 1.72E−115 | A48 | Atp5g3 |
| Smlr1 | 2.43E−119 | 0.306487823 | 0.68 | 0.297 | 6.02E−115 | A48 | Smlr1 |
| 0610005C13Rik1 | 4.83E−119 | 0.397711899 | 0.944 | 0.655 | 1.20E−114 | A48 | 0610005C13Rik |
| Sec14l21 | 1.17E−118 | 0.388989397 | 0.98 | 0.844 | 2.90E−114 | A48 | Sec14l2 |
| Cox5a1 | 1.82E−118 | 0.430264839 | 0.938 | 0.701 | 4.51E−114 | A48 | Cox5a |
| Azgp1 | 2.02E−117 | 0.28364834 | 0.997 | 0.968 | 5.02E−113 | A48 | Azgp1 |
| Slc37a41 | 3.91E−117 | 0.421081968 | 0.858 | 0.511 | 9.70E−113 | A48 | Slc37a4 |
| Pbld11 | 6.09E−117 | 0.398937692 | 0.881 | 0.559 | 1.51E−112 | A48 | Pbld1 |
| Abhd14b1 | 6.78E−116 | 0.41043543 | 0.885 | 0.565 | 1.68E−111 | A48 | Abhd14b |
| Sh3glb1 | 1.76E−115 | 0.32589137 | 0.695 | 0.317 | 4.36E−111 | A48 | Sh3glb1 |
| Acat1 | 9.40E−114 | 0.377331408 | 0.978 | 0.831 | 2.33E−109 | A48 | Acat1 |
| Armc1 | 1.07E−113 | 0.2587072 | 0.632 | 0.263 | 2.66E−109 | A48 | Armc1 |
| F12 | 3.14E−112 | 0.384983296 | 0.966 | 0.813 | 7.79E−108 | A48 | F12 |
| Sdhd1 | 6.90E−112 | 0.48488077 | 0.891 | 0.652 | 1.71E−107 | A48 | Sdhd |
| Atp5j1 | 7.69E−112 | 0.457478416 | 0.916 | 0.7 | 1.91E−107 | A48 | Atp5j |
| Acadl1 | 1.25E−111 | 0.387890841 | 0.896 | 0.603 | 3.10E−107 | A48 | Acadl1 |
| Tcea31 | 5.25E−111 | 0.393446905 | 0.825 | 0.466 | 1.30E−106 | A48 | Tcea3 |
| Shmt11 | 1.48E−109 | 0.38266243 | 0.882 | 0.562 | 3.67E−105 | A48 | Shmt1 |
| Cyp3a59 | 4.56E−109 | 0.26420294 | 0.723 | 0.324 | 1.13E−104 | A48 | Cyp3a59 |
| Sult1a11 | 1.06E−108 | 0.38558712 | 0.963 | 0.758 | 2.64E−104 | A48 | Sult1a1 |
| Plin51 | 1.53E−108 | 0.326445383 | 0.788 | 0.392 | 3.79E−104 | A48 | Plin5 |
| Atp5k1 | 3.01E−107 | 0.40741303 | 0.935 | 0.666 | 7.47E−103 | A48 | Atp5k |
| Sord1 | 9.47E−107 | 0.376343552 | 0.987 | 0.915 | 2.35E−102 | A48 | Sord |
| Park7 | 1.57E−106 | 0.402994356 | 0.953 | 0.765 | 3.90E−102 | A48 | Park7 |
| Gstt2 | 2.12E−104 | 0.323388931 | 0.754 | 0.375 | 5.26E−100 | A48 | Gstt2 |
| Pbld2 | 1.70E−103 | 0.361852773 | 0.891 | 0.571 | 4.23E−99 | A48 | Pbld2 |
| Abcc3 | 2.28E−103 | 0.407473287 | 0.859 | 0.548 | 5.65E−99 | A48 | Abcc3 |
| Yipf3 | 2.68E−103 | 0.270153738 | 0.68 | 0.314 | 6.66E−99 | A48 | Yipf3 |
| Brap | 6.59E−102 | 0.339491607 | 0.829 | 0.478 | 1.64E−97 | A48 | Brap |
| Ppara1 | 6.71E−101 | 0.344157474 | 0.831 | 0.469 | 1.67E−96 | A48 | Ppara |
| Cyp2d26 | 1.85E−100 | 0.329292761 | 0.982 | 0.902 | 4.58E−96 | A48 | Cyp2d26 |
| Sult1d11 | 4.70E−100 | 0.52821451 | 0.936 | 0.668 | 1.17E−95 | A48 | Sult1d1 |
| Aspdh | 8.32E−100 | 0.325986003 | 0.776 | 0.415 | 2.07E−95 | A48 | Aspdh |
| St3gal52 | 4.74E−99 | 0.376555805 | 0.914 | 0.607 | 1.18E−94 | A48 | St3gal5 |
| Ddb1 | 5.85E−99 | 0.337307537 | 0.83 | 0.482 | 1.45E−94 | A48 | Ddb1 |
| Ifnar2 | 7.04E−99 | 0.283328433 | 0.731 | 0.356 | 1.75E−94 | A48 | Ifnar2 |
| Mrpl43 | 1.55E−98 | 0.269199069 | 0.714 | 0.34 | 3.86E−94 | A48 | Mrpl43 |
| Ugt2b361 | 2.72E−98 | 0.35027496 | 0.99 | 0.91 | 6.76E−94 | A48 | Ugt2b36 |
| Acbd51 | 9.30E−98 | 0.351727998 | 0.943 | 0.704 | 2.31E−93 | A48 | Acbd5 |
| Lamp11 | 1.45E−97 | 0.337729586 | 0.982 | 0.888 | 3.59E−93 | A48 | Lamp1 |
| Ndufb22 | 2.71E−97 | 0.40700274 | 0.888 | 0.583 | 6.72E−93 | A48 | Ndufb2 |
| Acot12 | 4.00E−97 | 0.309136906 | 0.825 | 0.459 | 9.93E−93 | A48 | Acot12 |
| Dhrs41 | 5.62E−97 | 0.34200938 | 0.94 | 0.662 | 1.40E−92 | A48 | Dhrs4 |
| Tmem219 | 6.15E−97 | 0.264654785 | 0.664 | 0.306 | 1.53E−92 | A48 | Tmem219 |
| Akr1c19 | 1.41E−95 | 0.370522208 | 0.821 | 0.48 | 3.49E−91 | A48 | Akr1c19 |
| Pigr1 | 1.63E−95 | 0.422360865 | 0.977 | 0.937 | 4.05E−91 | A48 | Pigr |
| Hsd17b101 | 2.18E−95 | 0.445183738 | 0.927 | 0.681 | 5.42E−91 | A48 | Hsd17b10 |
| Gsta2 | 2.29E−95 | 0.273359973 | 0.638 | 0.288 | 5.70E−91 | A48 | Gsta2 |
| 1600002H07Rik | 3.87E−95 | 0.293949608 | 0.736 | 0.38 | 9.60E−91 | A48 | 1600002H07Rik |
| Upb11 | 5.45E−95 | 0.363137835 | 0.945 | 0.74 | 1.35E−90 | A48 | Upb1 |
| Sco2 | 6.00E−95 | 0.316967022 | 0.808 | 0.448 | 1.49E−90 | A48 | Sco2 |
| Slc27a2 | 5.90E−94 | 0.314567347 | 0.996 | 0.944 | 1.46E−89 | A48 | Slc27a2 |
| Ces2a | 6.30E−94 | 0.340367802 | 0.969 | 0.802 | 1.56E−89 | A48 | Ces2a |
| Acsl11 | 9.87E−94 | 0.366805004 | 0.978 | 0.878 | 2.45E−89 | A48 | Acsl1 |
| Ces1f | 2.92E−93 | 0.353237242 | 0.97 | 0.785 | 7.26E−89 | A48 | Ces1f |
| Usmg51 | 2.06E−92 | 0.417280438 | 0.755 | 0.427 | 5.11E−88 | A48 | Usmg5 |
| Asgr21 | 2.67E−91 | 0.343090019 | 0.879 | 0.573 | 6.64E−87 | A48 | Asgr2 |
| Cd1d1 | 3.09E−91 | 0.351797646 | 0.887 | 0.601 | 7.69E−87 | A48 | Cd1d1 |
| Dcxr2 | 1.59E−90 | 0.351763208 | 0.884 | 0.585 | 3.95E−86 | A48 | Dcxr |
| Cpt2 | 1.60E−90 | 0.321419422 | 0.807 | 0.466 | 3.96E−86 | A48 | Cpt2 |
| Rabac1 | 3.41E−90 | 0.334311521 | 0.807 | 0.468 | 8.47E−86 | A48 | Rabac1 |
| Zfp91 | 1.53E−89 | 0.322140173 | 0.855 | 0.531 | 3.79E−85 | A48 | Zfp91 |
| Aldoa | 2.71E−89 | 0.263527898 | 0.719 | 0.357 | 6.73E−85 | A48 | Aldoa |
| Cyp2c54 | 5.42E−88 | 0.442677432 | 0.896 | 0.613 | 1.35E−83 | A48 | Cyp2c54 |
| Gcdh1 | 7.24E−87 | 0.305992607 | 0.977 | 0.856 | 1.80E−82 | A48 | Gcdh |
| Glrx5 | 9.19E−87 | 0.330848831 | 0.886 | 0.573 | 2.28E−82 | A48 | Glrx5 |
| Snx3 | 3.01E−86 | 0.256221746 | 0.693 | 0.351 | 7.47E−82 | A48 | Snx3 |
| Atf5 | 2.84E−85 | 0.354363257 | 0.909 | 0.654 | 7.06E−81 | A48 | Atf5 |
| Creb3l3 | 3.83E−85 | 0.322778047 | 0.971 | 0.829 | 9.51E−81 | A48 | Creb3l3 |
| Lipa1 | 4.93E−85 | 0.327721132 | 0.904 | 0.64 | 1.23E−80 | A48 | Lipa |
| Stbd11 | 8.89E−85 | 0.266732483 | 0.725 | 0.373 | 2.21E−80 | A48 | Stbd1 |
| Ninj1 | 1.13E−84 | 0.354210049 | 0.874 | 0.601 | 2.81E−80 | A48 | Ninj1 |
| Ndufb101 | 3.61E−84 | 0.347097666 | 0.907 | 0.668 | 8.97E−80 | A48 | Ndufb10 |
| Eef1d | 3.98E−84 | 0.274526073 | 0.795 | 0.442 | 9.88E−80 | A48 | Eef1d |
| Cp | 7.52E−84 | 0.271474528 | 0.995 | 0.963 | 1.87E−79 | A48 | Cp |
| Ces1d2 | 8.69E−84 | 0.332413333 | 0.984 | 0.849 | 2.16E−79 | A48 | Ces1d |
| Pitpnb | 9.69E−84 | 0.272561149 | 0.795 | 0.447 | 2.41E−79 | A48 | Pitpnb |
| Camk2n11 | 1.18E−83 | 0.257842409 | 0.691 | 0.35 | 2.92E−79 | A48 | Camk2n1 |
| Mia3 | 1.40E−83 | 0.337358626 | 0.91 | 0.636 | 3.47E−79 | A48 | Mia3 |
| Aco2 | 3.85E−83 | 0.305389265 | 0.846 | 0.539 | 9.55E−79 | A48 | Aco2 |
| Galk1 | 6.87E−83 | 0.268631662 | 0.718 | 0.368 | 1.71E−78 | A48 | Galk1 |
| Gsto1 | 1.16E−82 | 0.339244623 | 0.818 | 0.508 | 2.87E−78 | A48 | Gsto1 |
| Gchfr2 | 1.22E−82 | 0.36759752 | 0.953 | 0.735 | 3.02E−78 | A48 | Gchfr |
| Cyp2c441 | 2.39E−82 | 0.392018859 | 0.924 | 0.718 | 5.94E−78 | A48 | Cyp2c44 |
| Ndufs7 | 4.90E−82 | 0.288168011 | 0.847 | 0.511 | 1.22E−77 | A48 | Ndufs7 |
| Fah1 | 6.81E−82 | 0.30390683 | 0.974 | 0.849 | 1.69E−77 | A48 | Fah |
| Ggnbp2 | 8.77E−82 | 0.282129685 | 0.818 | 0.466 | 2.18E−77 | A48 | Ggnbp2 |
| 11-Mar | 1.38E−81 | 0.29858018 | 0.984 | 0.881 | 3.42E−77 | A48 | 1-Mar |
| Anapc131 | 2.12E−81 | 0.276177491 | 0.768 | 0.429 | 5.28E−77 | A48 | Anapc13 |
| Ctsd | 2.25E−81 | 0.323706348 | 0.916 | 0.669 | 5.58E−77 | A48 | Ctsd |
| Mgll | 4.44E−81 | 0.319246054 | 0.844 | 0.518 | 1.10E−76 | A48 | Mgll |
| Hspa82 | 5.70E−81 | 0.327389675 | 0.952 | 0.776 | 1.41E−76 | A48 | Hspa8 |
| Gyk | 1.19E−80 | 0.265772985 | 0.778 | 0.424 | 2.95E−76 | A48 | Gyk |
| Grn1 | 1.60E−80 | 0.328395818 | 0.844 | 0.525 | 3.98E−76 | A48 | Grn |
| Pla2g12b | 2.48E−80 | 0.292440083 | 0.798 | 0.464 | 6.15E−76 | A48 | Pla2g12b |
| Slc2a21 | 2.96E−80 | 0.341623888 | 0.824 | 0.509 | 7.36E−76 | A48 | Slc2a2 |
| Sirt31 | 3.56E−80 | 0.258019667 | 0.724 | 0.387 | 8.85E−76 | A48 | Sirt3 |
| Gm49521 | 7.39E−80 | 0.298551252 | 0.824 | 0.491 | 1.84E−75 | A48 | Gm4952 |
| Rps27l2 | 1.61E−79 | 0.418112699 | 0.924 | 0.775 | 4.00E−75 | A48 | Rps27l |
| Suds3 | 5.33E−79 | 0.255930607 | 0.74 | 0.4 | 1.32E−74 | A48 | Suds3 |
| Klf9 | 5.80E−79 | 0.314463584 | 0.862 | 0.581 | 1.44E−74 | A48 | Klf9 |
| Wbp1l1 | 1.37E−78 | 0.334193985 | 0.91 | 0.684 | 3.39E−74 | A48 | Wbp1l |
| Ndufa131 | 3.89E−78 | 0.323295375 | 0.959 | 0.772 | 9.66E−74 | A48 | Ndufa13 |
| Cyp8b12 | 4.42E−78 | 0.355675896 | 0.911 | 0.629 | 1.10E−73 | A48 | Cyp8b1 |
| Mpc21 | 4.75E−78 | 0.384116346 | 0.953 | 0.817 | 1.18E−73 | A48 | Mpc2 |
| Tmem371 | 3.68E−77 | 0.266162015 | 0.786 | 0.441 | 9.14E−73 | A48 | Tmem37 |
| Ctage5 | 4.42E−77 | 0.331475 | 0.941 | 0.692 | 1.10E−72 | A48 | Ctage5 |
| Dctn2 | 8.59E−77 | 0.255512392 | 0.803 | 0.452 | 2.13E−72 | A48 | Dctn2 |
| Sfxn1 | 1.34E−76 | 0.339934169 | 0.822 | 0.528 | 3.32E−72 | A48 | Sfxn1 |
| Hagh1 | 1.43E−76 | 0.365459582 | 0.959 | 0.835 | 3.55E−72 | A48 | Hagh |
| Eci2 | 1.53E−76 | 0.309061425 | 0.944 | 0.714 | 3.80E−72 | A48 | Eci2 |
| Ubxn4 | 3.43E−76 | 0.296243889 | 0.893 | 0.599 | 8.52E−72 | A48 | Ubxn4 |
| Pccb | 5.27E−76 | 0.308534663 | 0.901 | 0.645 | 1.31E−71 | A48 | Pccb |
| Ndufs81 | 1.65E−75 | 0.338746392 | 0.854 | 0.589 | 4.09E−71 | A48 | Ndufs8 |
| D17Wsu92e | 2.26E−75 | 0.29017967 | 0.909 | 0.639 | 5.62E−71 | A48 | D17Wsu92e |
| Oplah | 7.71E−75 | 0.285269759 | 0.805 | 0.482 | 1.91E−70 | A48 | Oplah |
| Eif51 | 1.05E−74 | 0.294961471 | 0.953 | 0.74 | 2.61E−70 | A48 | Eif5 |
| Hsd11b1 | 2.39E−74 | 0.304583237 | 0.977 | 0.848 | 5.94E−70 | A48 | Hsd11b1 |
| Gas51 | 8.55E−74 | 0.273894232 | 0.803 | 0.485 | 2.12E−69 | A48 | Gas5 |
| Tgoln1 | 1.25E−73 | 0.317768081 | 0.891 | 0.617 | 3.10E−69 | A48 | Tgoln1 |
| Gm2a | 1.74E−73 | 0.286180358 | 0.827 | 0.504 | 4.33E−69 | A48 | Gm2a |
| 1100001G20Rik1 | 6.60E−73 | 0.334854645 | 0.993 | 0.979 | 1.64E−68 | A48 | 1100001G20Rik |
| Arhgap5 | 7.80E−73 | 0.264113051 | 0.852 | 0.509 | 1.94E−68 | A48 | Arhgap5 |
| Mtdh | 1.96E−72 | 0.252366839 | 0.819 | 0.475 | 4.88E−68 | A48 | Mtdh |
| Gpt | 2.68E−72 | 0.29324715 | 0.94 | 0.748 | 6.66E−68 | A48 | Gpt |
| Tspo1 | 3.58E−71 | 0.30737702 | 0.784 | 0.475 | 8.90E−67 | A48 | Tspo |
| Hsbp11 | 7.86E−71 | 0.284796107 | 0.792 | 0.502 | 1.95E−66 | A48 | Hsbp1 |
| Akr1d11 | 1.08E−70 | 0.28615505 | 0.852 | 0.546 | 2.68E−66 | A48 | Akr1d1 |
| Ugp21 | 5.70E−70 | 0.315665196 | 0.945 | 0.744 | 1.42E−65 | A48 | Ugp2 |
| Acadvl | 5.43E−69 | 0.274895665 | 0.959 | 0.791 | 1.35E−64 | A48 | Acadvl |
| Ndufv31 | 6.91E−68 | 0.263947933 | 0.868 | 0.574 | 1.72E−63 | A48 | Ndufv3 |
| Uqcrfs1 | 1.53E−67 | 0.307020216 | 0.949 | 0.781 | 3.79E−63 | A48 | Uqcrfs1 |
| Acadl | 2.72E−67 | 0.270468066 | 0.916 | 0.655 | 6.76E−63 | A48 | Acadl |
| Actn4 | 5.82E−66 | 0.253112911 | 0.789 | 0.489 | 1.44E−61 | A48 | Actn4 |
| Ubxn1 | 7.62E−66 | 0.253917187 | 0.86 | 0.572 | 1.89E−61 | A48 | Ubxn1 |
| Map2k2 | 8.71E−66 | 0.251107456 | 0.832 | 0.543 | 2.16E−61 | A48 | Map2k2 |
| Dgat21 | 1.45E−65 | 0.283867124 | 0.947 | 0.761 | 3.60E−61 | A48 | Dgat2 |
| Maob | 1.61E−65 | 0.289210898 | 0.889 | 0.609 | 4.01E−61 | A48 | Maob |
| Esd | 1.67E−65 | 0.309040682 | 0.935 | 0.734 | 4.15E−61 | A48 | Esd |
| Ccs1 | 1.06E−64 | 0.302390102 | 0.895 | 0.658 | 2.64E−60 | A48 | Ccs |
| Slc25a13 | 1.33E−64 | 0.274023596 | 0.924 | 0.678 | 3.29E−60 | A48 | Slc25a13 |
| Cyp2d101 | 2.32E−64 | 0.259489083 | 0.986 | 0.888 | 5.77E−60 | A48 | Cyp2d10 |
| Eci1 | 8.76E−64 | 0.280304871 | 0.914 | 0.67 | 2.18E−59 | A48 | Eci1 |
| Gpld1 | 1.63E−63 | 0.273116761 | 0.877 | 0.617 | 4.04E−59 | A48 | Gpld1 |
| Hal1 | 2.04E−63 | 0.383784717 | 0.908 | 0.734 | 5.06E−59 | A48 | Hal |
| Pglyrp2 | 2.50E−63 | 0.252927854 | 0.754 | 0.447 | 6.20E−59 | A48 | Pglyrp2 |
| Car8 | 6.69E−63 | 0.276457751 | 0.765 | 0.459 | 1.66E−58 | A48 | Car8 |
| Akr1c142 | 9.77E−63 | 0.29552724 | 0.92 | 0.685 | 2.43E−58 | A48 | Akr1c14 |
| Abca6 | 2.25E−62 | 0.271013657 | 0.849 | 0.569 | 5.59E−58 | A48 | Abca6 |
| Mst1 | 3.25E−62 | 0.256965699 | 0.81 | 0.517 | 8.06E−58 | A48 | Mst1 |
| Lpin21 | 3.66E−62 | 0.30814041 | 0.921 | 0.675 | 9.09E−58 | A48 | Lpin2 |
| Uroc1 | 5.97E−62 | 0.303202921 | 0.914 | 0.681 | 1.48E−57 | A48 | Uroc1 |
| Dpys | 7.13E−62 | 0.26481371 | 0.922 | 0.679 | 1.77E−57 | A48 | Dpys |
| Furin | 8.92E−62 | 0.256394896 | 0.781 | 0.481 | 2.21E−57 | A48 | Furin |
| Slc25a39 | 1.17E−61 | 0.282702178 | 0.938 | 0.783 | 2.91E−57 | A48 | Slc25a39 |
| Acaa1a1 | 1.53E−61 | 0.271027117 | 0.901 | 0.66 | 3.81E−57 | A48 | Acaa1a |
| Fkbp2 | 2.98E−61 | 0.253151747 | 0.841 | 0.539 | 7.40E−57 | A48 | Fkbp2 |
| Leap21 | 4.65E−61 | 0.374408905 | 0.833 | 0.566 | 1.15E−56 | A48 | Leap2 |
| Pim31 | 4.96E−60 | 0.270043644 | 0.845 | 0.554 | 1.23E−55 | A48 | Pim3 |
| Tmem14c1 | 6.44E−60 | 0.284049383 | 0.837 | 0.557 | 1.60E−55 | A48 | Tmem14c |
| Uqcr101 | 3.87E−59 | 0.275943715 | 0.943 | 0.764 | 9.61E−55 | A48 | Uqcr10 |
| Pemt1 | 3.70E−57 | 0.293943649 | 0.948 | 0.783 | 9.18E−53 | A48 | Pemt |
| Hadh1 | 5.81E−57 | 0.254909361 | 0.908 | 0.668 | 1.44E−52 | A48 | Hadh |
| Ndufb91 | 6.54E−57 | 0.284982242 | 0.982 | 0.855 | 1.62E−52 | A48 | Ndufb9 |
| Os9 | 7.96E−57 | 0.250595723 | 0.911 | 0.694 | 1.98E−52 | A48 | Os9 |
| Cyp2d22 | 9.72E−57 | 0.252444699 | 0.835 | 0.558 | 2.41E−52 | A48 | Cyp2d22 |
| Mug1 | 2.07E−56 | 0.266350935 | 0.998 | 0.977 | 5.13E−52 | A48 | Mug1 |
| Apoa42 | 2.43E−56 | 0.289005619 | 0.778 | 0.473 | 6.04E−52 | A48 | Apoa4 |
| Ces1e | 5.16E−56 | 0.252814514 | 0.864 | 0.565 | 1.28E−51 | A48 | Ces1e |
| Baat1 | 1.58E−55 | 0.250789569 | 0.954 | 0.738 | 3.93E−51 | A48 | Baat |
| Cideb2 | 2.02E−55 | 0.282394867 | 0.892 | 0.68 | 5.02E−51 | A48 | Cideb |
| Atp5d1 | 2.52E−55 | 0.255861997 | 0.935 | 0.724 | 6.25E−51 | A48 | Atp5d |
| Asl1 | 1.11E−54 | 0.348714359 | 0.953 | 0.8 | 2.75E−50 | A48 | Asl |
| Ephx1 | 1.24E−54 | 0.261130074 | 0.933 | 0.767 | 3.08E−50 | A48 | Ephx1 |
| Hadha | 1.83E−54 | 0.266045541 | 0.943 | 0.762 | 4.55E−50 | A48 | Hadha |
| Ehhadh | 1.74E−52 | 0.268061208 | 0.908 | 0.66 | 4.32E−48 | A48 | Ehhadh |
| Hint12 | 8.91E−51 | 0.299642398 | 0.954 | 0.812 | 2.21E−46 | A48 | Hint1 |
| Cyb52 | 1.29E−50 | 0.332815798 | 0.992 | 0.977 | 3.20E−46 | A48 | Cyb5 |
| Pcbd11 | 3.34E−47 | 0.255954233 | 0.932 | 0.818 | 8.30E−43 | A48 | Pcbd1 |
| Hsd17b6 | 6.76E−44 | 0.325224319 | 0.646 | 0.387 | 1.68E−39 | A48 | Hsd17b6 |
| Sds1 | 2.08E−43 | 0.355755983 | 0.799 | 0.625 | 5.18E−39 | A48 | Sds |
| Cyp2f2 | 1.20E−31 | 0.288977869 | 0.912 | 0.81 | 2.99E−27 | A48 | Cyp2f2 |
| Slco1b21 | 6.84E−29 | 0.302461427 | 0.996 | 0.931 | 1.70E−24 | A48 | Slco1b2 |
| Cyp2a52 | 5.71E−06 | 0.370640229 | 0.518 | 0.393 | 0.141821036 | A48 | Cyp2a5 |
| Cyp2c292 | 0.006241385 | 0.279136981 | 0.91 | 0.82 | 1 | A48 | Cyp2c29 |
| Cyp4a143 | 0 | 1.209065936 | 0.975 | 0.456 | 0 | A96 | Cyp4a14 |
| Apoc13 | 0 | 0.60439126 | 1 | 0.994 | 0 | A96 | Apoc1 |
| Zfp683 | 0 | 0.535767012 | 0.476 | 0.054 | 0 | A96 | Zfp683 |
| Cyp4a103 | 5.50E−304 | 1.099606992 | 0.974 | 0.58 | 1.36E−299 | A96 | Cyp4a10 |
| Angptl31 | 7.63E−290 | 0.72518632 | 0.998 | 0.945 | 1.89E−285 | A96 | Angptl3 |
| Apoa22 | 5.38E−255 | 0.433196336 | 1 | 0.999 | 1.34E−250 | A96 | Apoa2 |
| Apoe3 | 7.69E−254 | 0.435214924 | 1 | 1 | 1.91E−249 | A96 | Apoe |
| Cdo12 | 1.76E−207 | 0.535977967 | 0.998 | 0.957 | 4.37E−203 | A96 | Cdo1 |
| Mat1a3 | 8.71E−192 | 0.506975785 | 1 | 0.976 | 2.16E−187 | A96 | Mat1a |
| Chchd103 | 3.76E−191 | 0.565314195 | 0.994 | 0.932 | 9.34E−187 | A96 | Chchd10 |
| Ces1d3 | 1.37E−181 | 0.655727144 | 0.977 | 0.851 | 3.39E−177 | A96 | Ces1d |
| Apoh2 | 2.09E−169 | 0.393709699 | 0.998 | 0.982 | 5.20E−165 | A96 | Apoh |
| Ttpa1 | 6.68E−154 | 0.524445158 | 0.98 | 0.887 | 1.66E−149 | A96 | Ttpa |
| Ttr2 | 2.48E−153 | 0.326050936 | 1 | 0.999 | 6.15E−149 | A96 | Ttr |
| Hmgcs22 | 1.07E−147 | 0.394397485 | 0.996 | 0.973 | 2.66E−143 | A96 | Hmgcs2 |
| Cyp8b13 | 2.40E−140 | 0.659833251 | 0.897 | 0.633 | 5.96E−136 | A96 | Cyp8b1 |
| Ces1c2 | 4.90E−137 | 0.356546281 | 1 | 0.986 | 1.22E−132 | A96 | Ces1e |
| Cyp3a113 | 1.05E−126 | 0.384595952 | 0.999 | 0.997 | 2.60E−122 | A96 | Cyp3a11 |
| Rbp41 | 5.06E−125 | 0.310027462 | 1 | 0.996 | 1.26E−120 | A96 | Rbp4 |
| Retsat3 | 2.48E−120 | 0.5525706 | 0.957 | 0.774 | 6.16E−116 | A96 | Retsat |
| Ubc2 | 1.57E−116 | 0.480123165 | 0.968 | 0.854 | 3.90E−112 | A96 | Ubc |
| Rdh72 | 1.30E−109 | 0.351444842 | 0.995 | 0.959 | 3.22E−105 | A96 | Rdh7 |
| Cyp3a252 | 1.47E−109 | 0.421753505 | 0.981 | 0.887 | 3.66E−105 | A96 | Cyp3a25 |
| Slco1b22 | 1.65E−109 | 0.556869709 | 0.998 | 0.932 | 4.10E−105 | A96 | Slco1b2 |
| Cyp2c293 | 1.10E−108 | 0.755089243 | 0.962 | 0.817 | 2.74E−104 | A96 | Cyp2c29 |
| Acsl12 | 1.94E−103 | 0.438089942 | 0.977 | 0.879 | 4.82E−99 | A96 | Acsl1 |
| Cyp2c541 | 2.26E−99 | 0.556201457 | 0.86 | 0.619 | 5.61E−95 | A96 | Cyp2c54 |
| Atp5h3 | 2.14E−97 | 0.467241244 | 0.848 | 0.623 | 5.32E−93 | A96 | Atp5h |
| Fth11 | 4.12E−97 | 0.313702253 | 0.993 | 0.978 | 1.02E−92 | A96 | Fth1 |
| Apoc33 | 2.02E−93 | 0.303331054 | 1 | 0.983 | 5.01E−89 | A96 | Apoc3 |
| Ugt2b53 | 4.35E−89 | 0.339957413 | 0.986 | 0.94 | 1.08E−84 | A96 | Ugt2b5 |
| Creg11 | 4.42E−89 | 0.374731894 | 0.985 | 0.908 | 1.10E−84 | A96 | Creg1 |
| Cyp2c501 | 2.42E−88 | 0.50581129 | 0.943 | 0.789 | 6.01E−84 | A96 | Cyp2c50 |
| Acaa1b2 | 4.73E−88 | 0.414844819 | 0.986 | 0.887 | 1.17E−83 | A96 | Acaa1b |
| Apoc43 | 9.68E−87 | 0.310736478 | 0.998 | 0.974 | 2.40E−82 | A96 | Apoc4 |
| Acbd52 | 1.19E−85 | 0.452425437 | 0.87 | 0.712 | 2.96E−81 | A96 | Acbd5 |
| Sod11 | 2.96E−84 | 0.305519465 | 0.999 | 0.977 | 7.35E−80 | A96 | Sod1 |
| Slc27a21 | 3.51E−84 | 0.327297475 | 0.988 | 0.945 | 8.71E−80 | A96 | Slc27a2 |
| Amy12 | 2.18E−82 | 0.463903122 | 0.831 | 0.656 | 5.41E−78 | A96 | Amy1 |
| Cyp2c372 | 8.65E−78 | 0.601371289 | 0.71 | 0.425 | 2.15E−73 | A96 | Cyp2c37 |
| Crot1 | 2.32E−77 | 0.308615744 | 0.989 | 0.952 | 5.77E−73 | A96 | Crot |
| Nr1i33 | 3.31E−76 | 0.4562148 | 0.625 | 0.352 | 8.22E−72 | A96 | Nr1i3 |
| Sult1d12 | 6.18E−76 | 0.483383193 | 0.855 | 0.677 | 1.53E−71 | A96 | Sult1d1 |
| Ppp2r5a | 1.50E−75 | 0.458718371 | 0.764 | 0.569 | 3.73E−71 | A96 | Ppp2r5a |
| Hhex | 1.71E−75 | 0.418536303 | 0.543 | 0.286 | 4.25E−71 | A96 | Hhex |
| Hamp3 | 3.47E−74 | 0.478368592 | 0.92 | 0.793 | 8.61E−70 | A96 | Hamp |
| Chpt1 | 7.37E−74 | 0.430823819 | 0.848 | 0.677 | 1.83E−69 | A96 | Chpt1 |
| Clpx1 | 1.31E−73 | 0.408117525 | 0.884 | 0.751 | 3.25E−69 | A96 | Clpx |
| Cyp2b101 | 3.02E−73 | 0.335177862 | 0.68 | 0.351 | 7.50E−69 | A96 | Cyp2b10 |
| Abcb41 | 1.73E−72 | 0.424225528 | 0.878 | 0.706 | 4.31E−68 | A96 | Abcb4 |
| Aldh3a22 | 3.15E−72 | 0.604174759 | 0.687 | 0.432 | 7.81E−68 | A96 | Aldh3a2 |
| Cox6a13 | 2.09E−71 | 0.35746573 | 0.943 | 0.853 | 5.18E−67 | A96 | Cox6a1 |
| Pon11 | 4.31E−71 | 0.391882148 | 0.965 | 0.879 | 1.07E−66 | A96 | Pon1 |
| Insig22 | 1.33E−70 | 0.368288039 | 0.928 | 0.801 | 3.31E−66 | A96 | Insig2 |
| Cth1 | 3.72E−70 | 0.390224363 | 0.944 | 0.823 | 9.23E−66 | A96 | Cth |
| Atp5k2 | 1.24E−68 | 0.397998927 | 0.837 | 0.677 | 3.08E−64 | A96 | Atp5k |
| Ndrg22 | 2.58E−68 | 0.279303859 | 0.985 | 0.946 | 6.42E−64 | A96 | Ndrg2 |
| Mettl7b1 | 1.30E−67 | 0.330779976 | 0.949 | 0.884 | 3.24E−63 | A96 | Mettl7b |
| Mgst11 | 5.26E−65 | 0.251672309 | 1 | 0.995 | 1.31E−60 | A96 | Mgst1 |
| Snord1182 | 2.18E−63 | 0.439343487 | 0.564 | 0.324 | 5.41E−59 | A96 | Snord118 |
| Ephx22 | 1.06E−62 | 0.284030828 | 0.968 | 0.908 | 2.63E−58 | A96 | Ephx2 |
| Bhmt1 | 2.63E−62 | 0.268226692 | 0.991 | 0.947 | 6.54E−58 | A96 | Bhmt |
| Cyb53 | 3.62E−62 | 0.275263191 | 1 | 0.977 | 9.00E−58 | A96 | Cyb5 |
| Rps27l3 | 4.23E−62 | 0.345949131 | 0.909 | 0.778 | 1.05E−57 | A96 | Rps27l |
| Hmgcl1 | 1.14E−61 | 0.364377659 | 0.871 | 0.722 | 2.83E−57 | A96 | Hmgcl |
| Gsta21 | 7.73E−60 | 0.39758707 | 0.522 | 0.301 | 1.92E−55 | A96 | Gsta2 |
| Sc4mol1 | 1.00E−59 | 0.397503269 | 0.781 | 0.603 | 2.49E−55 | A96 | Sc4mol |
| Pex11a | 1.54E−59 | 0.461213321 | 0.7 | 0.487 | 3.81E−55 | A96 | Pex11a |
| Hspa83 | 1.87E−57 | 0.334752875 | 0.905 | 0.782 | 4.66E−53 | A96 | Hspa8 |
| Ube2r22 | 3.50E−57 | 0.373950286 | 0.747 | 0.565 | 8.69E−53 | A96 | Ube2r2 |
| Lipa2 | 4.84E−56 | 0.364686822 | 0.791 | 0.651 | 1.20E−51 | A96 | Lipa |
| Csad2 | 7.79E−55 | 0.455104263 | 0.843 | 0.641 | 1.93E−50 | A96 | Csad |
| Acat11 | 7.95E−55 | 0.330094254 | 0.917 | 0.837 | 1.97E−50 | A96 | Acat1 |
| Baat2 | 8.13E−55 | 0.351413298 | 0.862 | 0.747 | 2.02E−50 | A96 | Baat |
| Tmem56 | 3.20E−54 | 0.357155569 | 0.812 | 0.642 | 7.96E−50 | A96 | Tmem56 |
| Slc10a12 | 4.49E−53 | 0.309448919 | 0.936 | 0.847 | 1.12E−48 | A96 | Slc10a1 |
| Acot11 | 4.63E−53 | 0.35120599 | 0.459 | 0.24 | 1.15E−48 | A96 | Acot1 |
| Bnip33 | 6.74E−53 | 0.396644557 | 0.716 | 0.534 | 1.67E−48 | A96 | Bnip3 |
| Aldh1a11 | 1.69E−52 | 0.290873161 | 0.994 | 0.953 | 4.19E−48 | A96 | Aldh1a1 |
| Ahcy2 | 3.19E−52 | 0.276868815 | 0.934 | 0.864 | 7.91E−48 | A96 | Ahcy |
| Gstt31 | 4.66E−52 | 0.35168382 | 0.541 | 0.323 | 1.16E−47 | A96 | Gstt3 |
| Sec14l22 | 2.20E−51 | 0.283306885 | 0.939 | 0.849 | 5.47E−47 | A96 | Sec14l2 |
| Gas52 | 1.11E−50 | 0.359650606 | 0.68 | 0.498 | 2.76E−46 | A96 | Gas5 |
| Gabarapl1 | 7.02E−50 | 0.3228272 | 0.881 | 0.777 | 1.74E−45 | A96 | Gabarapl1 |
| Decr21 | 7.27E−49 | 0.330020285 | 0.813 | 0.664 | 1.81E−44 | A96 | Decr2 |
| Ndufa71 | 5.85E−48 | 0.315860633 | 0.818 | 0.68 | 1.45E−43 | A96 | Ndufa7 |
| Aldh9a11 | 3.79E−47 | 0.300984838 | 0.884 | 0.784 | 9.42E−43 | A96 | Aldh9a1 |
| Acot4 | 1.70E−45 | 0.256272851 | 0.328 | 0.149 | 4.23E−41 | A96 | Acot4 |
| Cyp2e12 | 2.08E−45 | 0.467377427 | 0.93 | 0.811 | 5.16E−41 | A96 | Cyp2e1 |
| Hsd17b102 | 3.65E−45 | 0.331127034 | 0.841 | 0.69 | 9.06E−41 | A96 | Hsd17b10 |
| Gyk1 | 4.15E−45 | 0.328560139 | 0.616 | 0.441 | 1.03E−40 | A96 | Gyk |
| Asl2 | 4.43E−45 | 0.29391729 | 0.896 | 0.806 | 1.10E−40 | A96 | Asl |
| Slco1a41 | 9.51E−45 | 0.35964429 | 0.507 | 0.311 | 2.36E−40 | A96 | Slco1a4 |
| Akr1d12 | 1.27E−44 | 0.320114687 | 0.721 | 0.56 | 3.16E−40 | A96 | Akr1d1 |
| Abcc31 | 1.93E−44 | 0.332981906 | 0.733 | 0.561 | 4.80E−40 | A96 | Abcc3 |
| Kif1b1 | 2.02E−44 | 0.335011009 | 0.686 | 0.499 | 5.03E−40 | A96 | Kif1b |
| Sord2 | 2.10E−44 | 0.260491087 | 0.969 | 0.917 | 5.21E−40 | A96 | Sord |
| Sult1a12 | 2.75E−44 | 0.301146751 | 0.881 | 0.767 | 6.82E−40 | A96 | Sult1a1 |
| Dpys1 | 9.36E−44 | 0.288671839 | 0.824 | 0.689 | 2.32E−39 | A96 | Dpys |
| Eif52 | 1.10E−43 | 0.271819491 | 0.879 | 0.748 | 2.73E−39 | A96 | Eif5 |
| G0s22 | 1.78E−43 | 0.311001159 | 0.525 | 0.315 | 4.41E−39 | A96 | G0s2 |
| Ctage51 | 2.81E−43 | 0.312935082 | 0.837 | 0.703 | 6.98E−39 | A96 | Ctage5 |
| 0610005C13Rik2 | 4.35E−43 | 0.300794029 | 0.816 | 0.668 | 1.08E−38 | A96 | 0610005C13Rik |
| Hmgcs11 | 4.61E−43 | 0.301665601 | 0.858 | 0.716 | 1.14E−38 | A96 | Hmgcs1 |
| Ugp22 | 7.52E−42 | 0.303666951 | 0.858 | 0.753 | 1.87E−37 | A96 | Ugp2 |
| Mia31 | 8.38E−42 | 0.315853543 | 0.81 | 0.647 | 2.08E−37 | A96 | Mia3 |
| Ndufb92 | 1.14E−41 | 0.269298954 | 0.951 | 0.859 | 2.83E−37 | A96 | Ndufb9 |
| Pbld12 | 1.54E−41 | 0.307893385 | 0.723 | 0.575 | 3.82E−37 | A96 | Pbld1 |
| Serpinf11 | 2.07E−41 | 0.262905272 | 0.934 | 0.861 | 5.15E−37 | A96 | Serpinf1 |
| Gm49522 | 2.95E−41 | 0.320671041 | 0.663 | 0.507 | 7.34E−37 | A96 | Gm4952 |
| Hpgd1 | 3.39E−41 | 0.349307092 | 0.588 | 0.404 | 8.42E−37 | A96 | Hpgd |
| Cyp7a12 | 3.62E−41 | 0.311335446 | 0.398 | 0.205 | 8.98E−37 | A96 | Cyp7a1 |
| Cd1d11 | 6.77E−41 | 0.301788074 | 0.747 | 0.615 | 1.68E−36 | A96 | Cd1d1 |
| Prlr | 9.98E−41 | 0.301923874 | 0.574 | 0.38 | 2.48E−36 | A96 | Prlr |
| Rdh16 | 1.14E−40 | 0.351901508 | 0.494 | 0.306 | 2.82E−36 | A96 | Rdh16 |
| Cox5a2 | 2.78E−40 | 0.296306496 | 0.813 | 0.713 | 6.92E−36 | A96 | Cox5a |
| Gadd45b | 9.36E−40 | 0.342036421 | 0.452 | 0.264 | 2.32E−35 | A96 | Gadd45b |
| Rdx2 | 2.27E−39 | 0.281003468 | 0.824 | 0.683 | 5.64E−35 | A96 | Rdx |
| Slc25a201 | 2.93E−39 | 0.295187748 | 0.671 | 0.53 | 7.28E−35 | A96 | Slc25a20 |
| Decr13 | 5.27E−39 | 0.300003168 | 0.808 | 0.694 | 1.31E−34 | A96 | Decr1 |
| Slc40a12 | 8.03E−39 | 0.286363927 | 0.551 | 0.367 | 1.99E−34 | A96 | Slc40a1 |
| Cxcl121 | 1.08E−38 | 0.300468634 | 0.635 | 0.477 | 2.69E−34 | A96 | Cxcl12 |
| Tob11 | 1.08E−38 | 0.301044665 | 0.649 | 0.492 | 2.69E−34 | A96 | Tob1 |
| Ces1f1 | 1.95E−38 | 0.278175079 | 0.895 | 0.793 | 4.83E−34 | A96 | Ces1f |
| Arl4a | 4.10E−38 | 0.293413307 | 0.517 | 0.333 | 1.02E−33 | A96 | Arl4a |
| Tsc22d3 | 4.12E−38 | 0.318115028 | 0.534 | 0.353 | 1.02E−33 | A96 | Tsc22d3 |
| Apoc21 | 5.51E−38 | 0.287194802 | 0.901 | 0.828 | 1.37E−33 | A96 | Apoc2 |
| Cyp2c681 | 6.33E−38 | 0.311687849 | 0.695 | 0.524 | 1.57E−33 | A96 | Cyp2c68 |
| Slc4a41 | 4.04E−37 | 0.29162163 | 0.609 | 0.43 | 1.00E−32 | A96 | Slc4a4 |
| Gclm | 4.44E−37 | 0.269048382 | 0.865 | 0.751 | 1.10E−32 | A96 | Gclm |
| Hsd17b7 | 5.55E−37 | 0.30226456 | 0.56 | 0.384 | 1.38E−32 | A96 | Hsd17b7 |
| Ech12 | 8.48E−37 | 0.250879529 | 0.917 | 0.814 | 2.10E−32 | A96 | Ech1 |
| Acot121 | 1.14E−36 | 0.280173953 | 0.65 | 0.477 | 2.83E−32 | A96 | Acot12 |
| Etfdh | 2.18E−36 | 0.27680923 | 0.811 | 0.707 | 5.40E−32 | A96 | Etfdh |
| Ndufa31 | 2.39E−36 | 0.276535456 | 0.737 | 0.602 | 5.93E−32 | A96 | Ndufa3 |
| Tgoln11 | 2.68E−36 | 0.296779403 | 0.756 | 0.631 | 6.66E−32 | A96 | Tgoln1 |
| Lcat2 | 3.69E−36 | 0.251857226 | 0.886 | 0.805 | 9.16E−32 | A96 | Lcat |
| Ephx11 | 3.81E−36 | 0.252026139 | 0.879 | 0.773 | 9.45E−32 | A96 | Ephx1 |
| Zfp911 | 8.05E−36 | 0.325520389 | 0.693 | 0.547 | 2.00E−31 | A96 | Zfp91 |
| Ndufb23 | 1.78E−35 | 0.300482948 | 0.731 | 0.599 | 4.43E−31 | A96 | Ndufb2 |
| D4Wsu53e | 3.13E−35 | 0.292450804 | 0.597 | 0.419 | 7.76E−31 | A96 | D4Wsu53e |
| Apol7a1 | 5.39E−35 | 0.302856549 | 0.559 | 0.392 | 1.34E−30 | A96 | Apol7a |
| Mtdh1 | 1.22E−34 | 0.283760224 | 0.649 | 0.492 | 3.03E−30 | A96 | Mtdh |
| Cisd11 | 2.63E−34 | 0.28202591 | 0.781 | 0.674 | 6.53E−30 | A96 | Cisd1 |
| Slc25a102 | 6.62E−34 | 0.260509641 | 0.701 | 0.552 | 1.64E−29 | A96 | Slc25a10 |
| Sdhc1 | 8.91E−34 | 0.272202687 | 0.76 | 0.66 | 2.21E−29 | A96 | Sdhc |
| Aqp91 | 1.11E−33 | 0.290066048 | 0.801 | 0.679 | 2.76E−29 | A96 | Aqp9 |
| Crls1 | 1.54E−33 | 0.255032135 | 0.447 | 0.281 | 3.83E−29 | A96 | Crls1 |
| Aplp2 | 2.37E−33 | 0.256928992 | 0.826 | 0.696 | 5.89E−29 | A96 | Aplp2 |
| Tmed51 | 4.31E−33 | 0.256258347 | 0.714 | 0.572 | 1.07E−28 | A96 | Tmed5 |
| Ces1e1 | 1.15E−32 | 0.310850144 | 0.71 | 0.581 | 2.85E−28 | A96 | Ces1e |
| Dhdh1 | 6.97E−32 | 0.259307598 | 0.734 | 0.62 | 1.73E−27 | A96 | Dhdh |
| Nfe2l2 | 1.68E−31 | 0.251281214 | 0.465 | 0.294 | 4.17E−27 | A96 | Nfe2l2 |
| Cpeb4 | 3.46E−31 | 0.299434499 | 0.527 | 0.371 | 8.60E−27 | A96 | Cpeb4 |
| 8430408G22Rik1 | 3.96E−31 | 0.277441955 | 0.559 | 0.389 | 9.83E−27 | A96 | 8430408G22Rik |
| Cd36 | 4.36E−31 | 0.278212926 | 0.299 | 0.153 | 1.08E−26 | A96 | Cd36 |
| Prkd32 | 4.40E−31 | 0.259284079 | 0.682 | 0.541 | 1.09E−26 | A96 | Prkd3 |
| Brap1 | 1.04E−30 | 0.25715186 | 0.642 | 0.497 | 2.59E−26 | A96 | Brap |
| Mocs21 | 1.07E−30 | 0.26658769 | 0.737 | 0.615 | 2.66E−26 | A96 | Mocs2 |
| Agpat6 | 1.18E−30 | 0.275279281 | 0.743 | 0.61 | 2.93E−26 | A96 | Agpat6 |
| Tmem106b | 1.28E−30 | 0.260426336 | 0.727 | 0.602 | 3.17E−26 | A96 | Tmem106b |
| Pabpc12 | 1.03E−29 | 0.252666197 | 0.766 | 0.655 | 2.55E−25 | A96 | Pabpc1 |
| D17Wsu92e1 | 1.06E−29 | 0.257998282 | 0.771 | 0.653 | 2.62E−25 | A96 | D17Wsu92e |
| Cyp2c442 | 1.54E−29 | 0.264049939 | 0.828 | 0.728 | 3.82E−25 | A96 | Cyp2c44 |
| Dcxr3 | 1.66E−29 | 0.252198764 | 0.735 | 0.6 | 4.13E−25 | A96 | Dcxr |
| Ptp4a2 | 4.47E−29 | 0.263379468 | 0.691 | 0.573 | 1.11E−24 | A96 | Ptp4a2 |
| Sdhd2 | 4.97E−29 | 0.260964394 | 0.775 | 0.664 | 1.23E−24 | A96 | Sdhd |
| Ehhadh1 | 2.37E−28 | 0.254528118 | 0.78 | 0.673 | 5.88E−24 | A96 | Ehhadh |
| Pdk1 | 3.43E−28 | 0.255751279 | 0.438 | 0.283 | 8.51E−24 | A96 | Pdk1 |
| Cyp2a53 | 8.31E−28 | 0.346687234 | 0.581 | 0.389 | 2.06E−23 | A96 | Cyp2a5 |
| Cebpb3 | 4.85E−27 | 0.251454788 | 0.585 | 0.443 | 1.20E−22 | A96 | Cebpb |
| Usmg52 | 7.28E−27 | 0.256460761 | 0.59 | 0.443 | 1.81E−22 | A96 | Usmg5 |
| Rgn2 | 1.30E−26 | 0.290981568 | 0.899 | 0.808 | 3.23E−22 | A96 | Rgn |
| Cede107 | 7.35E−26 | 0.26255392 | 0.59 | 0.458 | 1.82E−21 | A96 | Cede107 |
| Fmo1 | 3.25E−25 | 0.253513023 | 0.606 | 0.456 | 8.06E−21 | A96 | Fmo1 |
| Etnppl3 | 1.26E−24 | 0.410971098 | 0.677 | 0.569 | 3.13E−20 | A96 | Etnppl |
| Hamp2 | 5.46E−24 | 0.259654924 | 0.203 | 0.093 | 1.35E−19 | A96 | Hamp2 |
| Cpox1 | 1.04E−21 | 0.25616093 | 0.634 | 0.522 | 2.58E−17 | A96 | Cpox |
| Oat2 | 3.84E−20 | 0.321311893 | 0.653 | 0.508 | 9.53E−16 | A96 | Oat |
| Mt24 | 0 | 1.789997157 | 1 | 0.555 | 0 | PHX3 | Mt2 |
| Mt14 | 0 | 1.628319565 | 1 | 0.709 | 0 | PHX3 | Mt1 |
| Steap44 | 0 | 1.546860615 | 0.973 | 0.366 | 0 | PHX3 | Steap4 |
| Lpin13 | 0 | 1.189493717 | 0.94 | 0.39 | 0 | PHX3 | Lpin1 |
| Serpina3n3 | 0 | 1.186862923 | 0.992 | 0.723 | 0 | PHX3 | Serpina3n |
| Thrsp3 | 0 | 1.107456378 | 0.98 | 0.578 | 0 | PHX3 | Thrsp |
| Cxcl13 | 0 | 1.09269701 | 0.919 | 0.364 | 0 | PHX3 | Cxcl1 |
| Igfbp13 | 0 | 1.083068625 | 0.99 | 0.73 | 0 | PHX3 | Igfbp1 |
| Tacc22 | 0 | 0.799081935 | 0.707 | 0.148 | 0 | PHX3 | Tacc2 |
| Scd11 | 0 | 0.743251417 | 0.998 | 0.936 | 0 | PHX3 | Scd1 |
| Fam134b1 | 0 | 0.727776721 | 0.876 | 0.482 | 0 | PHX3 | Fam134b |
| St3gal53 | 0 | 0.726028159 | 0.915 | 0.574 | 0 | PHX3 | St3gal5 |
| Il1r13 | 0 | 0.711336628 | 0.8 | 0.322 | 0 | PHX3 | Il1r1 |
| B3galt12 | 0 | 0.705093027 | 0.634 | 0.225 | 0 | PHX3 | B3galt1 |
| H3f3b3 | 0 | 0.694554678 | 0.831 | 0.454 | 0 | PHX3 | H3f3b |
| Nnmt3 | 0 | 0.668908642 | 0.97 | 0.767 | 0 | PHX3 | Nnmt |
| Slc38a2 | 0 | 0.666231739 | 0.874 | 0.49 | 0 | PHX3 | Slc38a2 |
| Tat2 | 0 | 0.643729389 | 0.998 | 0.929 | 0 | PHX3 | Tat |
| n-R5-8s13 | 0 | 0.641983104 | 0.805 | 0.246 | 0 | PHX3 | n-R5-8s1 |
| Slc39a142 | 0 | 0.580960968 | 0.849 | 0.469 | 0 | PHX3 | Slc39a14 |
| Fkbp52 | 0 | 0.562874909 | 0.665 | 0.218 | 0 | PHX3 | Fkbp5 |
| Plin23 | 0 | 0.560892991 | 0.952 | 0.739 | 0 | PHX3 | Plin2 |
| Slc25a472 | 0 | 0.556992763 | 0.994 | 0.905 | 0 | PHX3 | Slc25a47 |
| Gm246013 | 0 | 0.531358312 | 0.661 | 0.173 | 0 | PHX3 | Gm24601 |
| Serpina1e1 | 0 | 0.510250179 | 1 | 0.994 | 0 | PHX3 | Serpina1e |
| Cldn14 | 0 | 0.480249207 | 0.505 | 0.109 | 0 | PHX3 | Cldn14 |
| Fga3 | 0 | 0.479585344 | 1 | 0.996 | 0 | PHX3 | Fga |
| Fgg3 | 0 | 0.468687867 | 1 | 0.995 | 0 | PHX3 | Fgg |
| Acox12 | 0 | 0.456568318 | 0.998 | 0.962 | 0 | PHX3 | Acox1 |
| Rcan11 | 0 | 0.434158745 | 0.522 | 0.133 | 0 | PHX3 | Rcan1 |
| Aldob1 | 0 | 0.415637015 | 1 | 0.996 | 0 | PHX3 | Aldob |
| Fgb3 | 0 | 0.400821016 | 1 | 0.999 | 0 | PHX3 | Fgb |
| Pfkfb3 | 0 | 0.388386728 | 0.421 | 0.063 | 0 | PHX3 | Pfkfb3 |
| Fbf1 | 0 | 0.292192664 | 0.336 | 0.034 | 0 | PHX3 | Fbf1 |
| Rapgef42 | 4.60E−302 | 0.581053171 | 0.836 | 0.461 | 1.14E−297 | PHX3 | Rapgef4 |
| Upp22 | 6.10E−292 | 0.572267247 | 0.934 | 0.645 | 1.52E−287 | PHX3 | Upp2 |
| Arhgef26 | 3.60E−291 | 0.49916866 | 0.67 | 0.272 | 8.95E−287 | PHX3 | Arhgef26 |
| Aass | 2.84E−286 | 0.585116267 | 0.965 | 0.756 | 7.05E−282 | PHX3 | Aass |
| Gadd45g2 | 1.61E−283 | 0.563511755 | 0.899 | 0.56 | 4.01E−279 | PHX3 | Gadd45g |
| Fmo5 | 3.25E−279 | 0.495494591 | 0.991 | 0.879 | 8.06E−275 | PHX3 | Fmo5 |
| Bcl3 | 1.59E−278 | 0.366999466 | 0.479 | 0.134 | 3.94E−274 | PHX3 | Bcl3 |
| Tiparp1 | 3.77E−277 | 0.440747263 | 0.485 | 0.14 | 9.36E−273 | PHX3 | Tiparp |
| Saa43 | 3.26E−273 | 0.499433314 | 0.981 | 0.79 | 8.10E−269 | PHX3 | Saa4 |
| Txnip | 8.19E−267 | 0.534615839 | 0.741 | 0.346 | 2.03E−262 | PHX3 | Txnip |
| Errfi1 | 9.98E−264 | 0.409765728 | 0.998 | 0.928 | 2.48E−259 | PHX3 | Errfi1 |
| Scp22 | 4.34E−260 | 0.350885679 | 0.997 | 0.972 | 1.08E−255 | PHX3 | Scp2 |
| Snx10 | 1.61E−256 | 0.472894384 | 0.63 | 0.263 | 3.99E−252 | PHX3 | Snx10 |
| Gpcpd11 | 3.99E−254 | 0.553228383 | 0.778 | 0.419 | 9.92E−250 | PHX3 | Gpcpd1 |
| Lcn24 | 5.86E−253 | 0.318374231 | 0.815 | 0.323 | 1.46E−248 | PHX3 | Lcn2 |
| Btg21 | 1.73E−250 | 0.577772828 | 0.708 | 0.344 | 4.30E−246 | PHX3 | Btg2 |
| 2010003K11Rik1 | 5.13E−243 | 0.306670297 | 0.412 | 0.107 | 1.27E−238 | PHX3 | 2010003K11Rik |
| Serpina3m1 | 1.19E−239 | 0.488987274 | 0.961 | 0.784 | 2.95E−235 | PHX3 | Serpina3m |
| Gstp13 | 2.19E−238 | 0.482301792 | 0.962 | 0.741 | 5.45E−234 | PHX3 | Gstp1 |
| Etnk21 | 3.85E−238 | 0.519747051 | 0.918 | 0.685 | 9.57E−234 | PHX3 | Etnk2 |
| Plin52 | 2.34E−236 | 0.455223479 | 0.736 | 0.361 | 5.81E−232 | PHX3 | Plin5 |
| Bach11 | 4.06E−233 | 0.423068365 | 0.589 | 0.237 | 1.01E−228 | PHX3 | Bach1 |
| Tdo2 | 3.68E−231 | 0.319972551 | 0.999 | 0.981 | 9.15E−227 | PHX3 | Tdo2 |
| Tifa | 2.38E−230 | 0.311085748 | 0.393 | 0.1 | 5.92E−226 | PHX3 | Tifa |
| Itih43 | 1.33E−228 | 0.354098265 | 0.999 | 0.968 | 3.30E−224 | PHX3 | Itih4 |
| Fam35a | 2.66E−225 | 0.437093621 | 0.642 | 0.292 | 6.61E−221 | PHX3 | Fam35a |
| Plk32 | 9.69E−225 | 0.430480294 | 0.644 | 0.287 | 2.41E−220 | PHX3 | Plk3 |
| Hspb82 | 2.27E−224 | 0.492739021 | 0.914 | 0.662 | 5.63E−220 | PHX3 | Hspb8 |
| Ptp4a1 | 2.29E−218 | 0.364094864 | 0.528 | 0.2 | 5.68E−214 | PHX3 | Ptp4a1 |
| Xbp12 | 4.38E−212 | 0.448896235 | 0.89 | 0.611 | 1.09E−207 | PHX3 | Xbp1 |
| Slc7a2 | 3.94E−210 | 0.446001839 | 0.964 | 0.762 | 9.78E−206 | PHX3 | Slc7a2 |
| Sds3 | 2.76E−209 | 0.764967866 | 0.868 | 0.592 | 6.84E−205 | PHX3 | Sds |
| Gpt21 | 3.87E−209 | 0.43436454 | 0.949 | 0.752 | 9.60E−205 | PHX3 | Gpt2 |
| Arg1 | 1.02E−200 | 0.315251526 | 1 | 0.978 | 2.54E−196 | PHX3 | Arg1 |
| Rnf103 | 7.01E−200 | 0.429306898 | 0.701 | 0.376 | 1.74E−195 | PHX3 | Rnf103 |
| Apoa52 | 2.12E−195 | 0.335415384 | 0.999 | 0.952 | 5.27E−191 | PHX3 | Apoa5 |
| Slc25a25 | 1.00E−194 | 0.424727464 | 0.945 | 0.745 | 2.49E−190 | PHX3 | Slc25a25 |
| Klf10 | 1.02E−194 | 0.26769517 | 0.388 | 0.114 | 2.53E−190 | PHX3 | Klf10 |
| Fh11 | 4.84E−194 | 0.45501042 | 0.915 | 0.685 | 1.20E−189 | PHX3 | Fh1 |
| Trib1 | 7.34E−194 | 0.467455716 | 0.705 | 0.386 | 1.82E−189 | PHX3 | Trib1 |
| Atf3 | 2.03E−192 | 0.484651606 | 0.56 | 0.24 | 5.04E−188 | PHX3 | Atf3 |
| Gjb22 | 1.51E−188 | 0.429397446 | 0.901 | 0.661 | 3.76E−184 | PHX3 | Gjb2 |
| Got11 | 1.69E−188 | 0.48064162 | 0.866 | 0.618 | 4.20E−184 | PHX3 | Got1 |
| 1810055G02Rik | 1.32E−186 | 0.352031321 | 0.528 | 0.217 | 3.27E−182 | PHX3 | 1810055G02Rik |
| Id2 | 2.54E−186 | 0.465018314 | 0.826 | 0.555 | 6.30E−182 | PHX3 | Id2 |
| Egfr3 | 6.93E−185 | 0.392521443 | 0.989 | 0.837 | 1.72E−180 | PHX3 | Egfr |
| Rgs162 | 1.91E−184 | 0.362782533 | 0.482 | 0.174 | 4.73E−180 | PHX3 | Rgs16 |
| Mtss11 | 4.80E−183 | 0.438122599 | 0.812 | 0.523 | 1.19E−178 | PHX3 | Mtss1 |
| Rhob | 2.92E−182 | 0.314794887 | 0.4 | 0.13 | 7.25E−178 | PHX3 | Rhob |
| Junb | 9.45E−182 | 0.446083855 | 0.788 | 0.495 | 2.35E−177 | PHX3 | Junb |
| Egr12 | 2.29E−180 | 0.492150366 | 0.79 | 0.491 | 5.68E−176 | PHX3 | Egr1 |
| Mbd1 | 9.76E−179 | 0.333504242 | 0.507 | 0.202 | 2.42E−174 | PHX3 | Mbd1 |
| Pck11 | 2.20E−178 | 0.304952819 | 0.999 | 0.975 | 5.47E−174 | PHX3 | Pck1 |
| Serpina103 | 1.39E−177 | 0.394340765 | 0.934 | 0.731 | 3.45E−173 | PHX3 | Serpina10 |
| Slc25a51 | 8.62E−177 | 0.328040909 | 0.522 | 0.218 | 2.14E−172 | PHX3 | Slc25a51 |
| Klf61 | 1.14E−176 | 0.258557871 | 0.346 | 0.097 | 2.82E−172 | PHX3 | Klf6 |
| Cdkn1a3 | 6.07E−174 | 0.321419697 | 0.572 | 0.245 | 1.51E−169 | PHX3 | Cdkn1a |
| Shb | 6.63E−172 | 0.354830541 | 0.589 | 0.281 | 1.65E−167 | PHX3 | Shb |
| Hnf4a1 | 3.33E−169 | 0.369123652 | 0.969 | 0.806 | 8.26E−165 | PHX3 | Hnf4a |
| Jun | 1.45E−168 | 0.455833119 | 0.864 | 0.61 | 3.60E−164 | PHX3 | Jun |
| Dak | 3.89E−168 | 0.429081642 | 0.867 | 0.599 | 9.65E−164 | PHX3 | Dak |
| Mup10 | 1.70E−164 | 0.315514103 | 0.965 | 0.779 | 4.21E−160 | PHX3 | Mup10 |
| Fasn | 1.97E−164 | 0.393405386 | 0.719 | 0.395 | 4.90E−160 | PHX3 | Fasn |
| Slc25a22 | 3.24E−162 | 0.348459422 | 0.649 | 0.339 | 8.04E−158 | PHX3 | Slc25a22 |
| B4galt1 | 3.41E−161 | 0.277865586 | 0.494 | 0.203 | 8.46E−157 | PHX3 | B4galt1 |
| Actb3 | 2.10E−156 | 0.283891315 | 0.99 | 0.906 | 5.20E−152 | PHX3 | Actb |
| Cyp4a12a2 | 3.54E−156 | 0.403038241 | 0.823 | 0.512 | 8.80E−152 | PHX3 | Cyp4a12a |
| Lrg11 | 4.69E−155 | 0.317155413 | 0.981 | 0.837 | 1.16E−150 | PHX3 | Lrg1 |
| Elovl6 | 7.53E−153 | 0.369426318 | 0.553 | 0.268 | 1.87E−148 | PHX3 | Elovl6 |
| Nfil31 | 4.61E−152 | 0.345982344 | 0.617 | 0.317 | 1.14E−147 | PHX3 | Nfil3 |
| G6pc2 | 3.85E−151 | 0.423823035 | 0.948 | 0.74 | 9.56E−147 | PHX3 | G6pc |
| Myo1e1 | 5.05E−151 | 0.313552851 | 0.551 | 0.251 | 1.25E−146 | PHX3 | Myo1e |
| Ivns1abp | 7.63E−151 | 0.358642904 | 0.688 | 0.394 | 1.89E−146 | PHX3 | Ivns1abp |
| Uox1 | 5.40E−149 | 0.269119908 | 0.998 | 0.966 | 1.34E−144 | PHX3 | Uox |
| Sephs23 | 2.80E−146 | 0.327490234 | 0.974 | 0.817 | 6.95E−142 | PHX3 | Sephs2 |
| Ier2 | 8.72E−146 | 0.427074433 | 0.763 | 0.509 | 2.17E−141 | PHX3 | Ier2 |
| B2m2 | 9.57E−146 | 0.274069023 | 0.997 | 0.953 | 2.38E−141 | PHX3 | B2m |
| Jmjd1c1 | 2.06E−144 | 0.322197078 | 0.516 | 0.238 | 5.11E−140 | PHX3 | Jmjd1c |
| Sdc41 | 2.93E−141 | 0.29285071 | 0.986 | 0.895 | 7.26E−137 | PHX3 | Sdc4 |
| Atp1b1 | 1.89E−138 | 0.34781274 | 0.567 | 0.293 | 4.69E−134 | PHX3 | Atp1b1 |
| Selenbp23 | 4.97E−138 | 0.374671601 | 0.986 | 0.828 | 1.23E−133 | PHX3 | Selenbp2 |
| Ccnl1 | 1.61E−137 | 0.31921767 | 0.526 | 0.253 | 3.99E−133 | PHX3 | Ccnl1 |
| Mfsd2a3 | 5.03E−136 | 0.28900787 | 0.563 | 0.263 | 1.25E−131 | PHX3 | Mfsd2a |
| Id31 | 3.10E−132 | 0.450224629 | 0.688 | 0.421 | 7.70E−128 | PHX3 | Id3 |
| Abca13 | 3.84E−131 | 0.324806561 | 0.756 | 0.465 | 9.53E−127 | PHX3 | Abca1 |
| Ddx3x1 | 1.66E−128 | 0.348123451 | 0.823 | 0.578 | 4.13E−124 | PHX3 | Ddx3x |
| Aox33 | 4.73E−128 | 0.334555042 | 0.989 | 0.876 | 1.18E−123 | PHX3 | Aox3 |
| Tns11 | 2.48E−121 | 0.270591692 | 0.524 | 0.257 | 6.16E−117 | PHX3 | Tns1 |
| Slco1a11 | 7.28E−121 | 0.323170693 | 0.927 | 0.71 | 1.81E−116 | PHX3 | Slco1a1 |
| Pik3r1 | 6.11E−119 | 0.299120271 | 0.577 | 0.313 | 1.52E−114 | PHX3 | Pik3r1 |
| Gfra1 | 2.01E−118 | 0.31861648 | 0.835 | 0.584 | 4.99E−114 | PHX3 | Gfra1 |
| Atl2 | 8.16E−117 | 0.288947906 | 0.674 | 0.404 | 2.03E−112 | PHX3 | Atl2 |
| Zfp36 | 1.13E−115 | 0.375886126 | 0.776 | 0.543 | 2.82E−111 | PHX3 | Zfp36 |
| Gck | 6.80E−113 | 0.272963666 | 0.557 | 0.289 | 1.69E−108 | PHX3 | Gck |
| Ulk1 | 1.04E−111 | 0.255629811 | 0.499 | 0.247 | 2.58E−107 | PHX3 | Ulk1 |
| Bhlhe40 | 2.78E−111 | 0.272247091 | 0.628 | 0.351 | 6.90E−107 | PHX3 | Bhlhe40 |
| Serpina122 | 3.41E−107 | 0.343116487 | 0.648 | 0.372 | 8.46E−103 | PHX3 | Serpina12 |
| Stat32 | 2.75E−106 | 0.254119857 | 0.701 | 0.431 | 6.83E−102 | PHX3 | Stat3 |
| Litaf | 1.58E−105 | 0.282863512 | 0.603 | 0.361 | 3.93E−101 | PHX3 | Litaf |
| Fos | 9.31E−103 | 0.379607371 | 0.785 | 0.559 | 2.31E−98 | PHX3 | Fos |
| Csad3 | 2.57E−102 | 0.409237969 | 0.872 | 0.611 | 6.38E−98 | PHX3 | Csad |
| Slc25a15 | 1.32E−99 | 0.287179656 | 0.931 | 0.782 | 3.27E−95 | PHX3 | Slc25a15 |
| Cpt1a | 3.31E−96 | 0.287909766 | 0.894 | 0.695 | 8.23E−92 | PHX3 | Cpt1a |
| Cyp2f21 | 1.47E−94 | 0.355492212 | 0.965 | 0.788 | 3.66E−90 | PHX3 | Cyp2f2 |
| Pim32 | 1.63E−93 | 0.292474208 | 0.764 | 0.54 | 4.04E−89 | PHX3 | Pim3 |
| Pnpla74 | 8.45E−93 | 0.269572352 | 0.807 | 0.571 | 2.10E−88 | PHX3 | Pnpla7 |
| Acly3 | 1.33E−90 | 0.279828458 | 0.715 | 0.471 | 3.30E−86 | PHX3 | Acly |
| Ifitm33 | 4.94E−89 | 0.257336347 | 0.958 | 0.819 | 1.23E−84 | PHX3 | Ifitm3 |
| Angptl41 | 7.69E−89 | 0.287033468 | 0.838 | 0.637 | 1.91E−84 | PHX3 | Angptl4 |
| Pklr | 8.66E−88 | 0.258258018 | 0.624 | 0.385 | 2.15E−83 | PHX3 | Pklr |
| Eci21 | 1.78E−87 | 0.251237147 | 0.896 | 0.699 | 4.41E−83 | PHX3 | Eci2 |
| Me1 | 3.42E−87 | 0.256971177 | 0.699 | 0.45 | 8.49E−83 | PHX3 | Me1 |
| Elovl5 | 9.27E−86 | 0.284139128 | 0.863 | 0.635 | 2.30E−81 | PHX3 | Elovl5 |
| Enpp2 | 3.09E−85 | 0.258210814 | 0.635 | 0.402 | 7.68E−81 | PHX3 | Enpp2 |
| Jak11 | 1.56E−81 | 0.253798387 | 0.648 | 0.415 | 3.88E−77 | PHX3 | Jak1 |
| Gls2 | 3.38E−77 | 0.286724421 | 0.868 | 0.648 | 8.39E−73 | PHX3 | Gls2 |
| Rcl1 | 3.70E−76 | 0.262666169 | 0.734 | 0.532 | 9.20E−72 | PHX3 | Rcl1 |
| Saa35 | 0 | 2.175493617 | 0.629 | 0.098 | 0 | PHX48 | Saa3 |
| Saa24 | 0 | 2.173589396 | 0.995 | 0.473 | 0 | PHX48 | Saa2 |
| Saa14 | 0 | 2.03134899 | 0.999 | 0.722 | 0 | PHX48 | Saa1 |
| Orm25 | 0 | 1.9624582 | 0.882 | 0.244 | 0 | PHX48 | Orm2 |
| Apcs3 | 0 | 1.668765328 | 0.922 | 0.677 | 0 | PHX48 | Apcs |
| Orm13 | 0 | 1.510438466 | 0.997 | 0.962 | 0 | PHX48 | Orm1 |
| Lcn25 | 0 | 1.392839951 | 0.862 | 0.363 | 0 | PHX48 | Lcn2 |
| Hpx3 | 0 | 1.146101117 | 1 | 0.998 | 0 | PHX48 | Hpx |
| Hp3 | 0 | 1.114046301 | 1 | 0.999 | 0 | PHX48 | Hp |
| Fgl13 | 0 | 1.043568641 | 0.967 | 0.872 | 0 | PHX48 | Fgl1 |
| Itih44 | 1.13E−213 | 0.645979807 | 0.99 | 0.972 | 2.82E−209 | PHX48 | Itih4 |
| Trf1 | 1.02E−212 | 0.482924139 | 0.997 | 0.998 | 2.54E−208 | PHX48 | Trf |
| Itih32 | 1.73E−208 | 0.781133024 | 0.963 | 0.938 | 4.29E−204 | PHX48 | Itih3 |
| Csf2ra | 4.36E−198 | 0.338511697 | 0.158 | 0.009 | 1.08E−193 | PHX48 | Csf2ra |
| C3 | 1.57E−185 | 0.56180364 | 0.998 | 0.995 | 3.91E−181 | PHX48 | C3 |
| Gm269244 | 5.19E−162 | 0.652363116 | 1 | 1 | 1.29E−157 | PHX48 | Gm26924 |
| Fam25c3 | 1.03E−139 | 0.967401342 | 0.653 | 0.383 | 2.56E−135 | PHX48 | Fam25c |
| D17H6S56E-5 | 3.62E−121 | 0.530503537 | 0.152 | 0.018 | 8.99E−117 | PHX48 | D17H6S56E-5 |
| Hmgcs13 | 4.47E−108 | 0.781596583 | 0.819 | 0.717 | 1.11E−103 | PHX48 | Hmgcs1 |
| Ambp | 3.32E−95 | 0.306730872 | 0.995 | 0.994 | 8.25E−91 | PHX48 | Ambp |
| Rnase42 | 1.43E−87 | 0.535044621 | 0.933 | 0.935 | 3.56E−83 | PHX48 | Rnase4 |
| Lrg12 | 3.00E−84 | 0.490737188 | 0.905 | 0.857 | 7.46E−80 | PHX48 | Lrg1 |
| Apoa44 | 1.51E−81 | 0.809620465 | 0.658 | 0.483 | 3.75E−77 | PHX48 | Apoa4 |
| Dhcr24 | 1.13E−73 | 0.513450385 | 0.862 | 0.863 | 2.81E−69 | PHX48 | Dhcr24 |
| Prtn3 | 1.78E−73 | 0.258105361 | 0.2 | 0.051 | 4.43E−69 | PHX48 | Prtn3 |
| Gstm34 | 2.36E−71 | 1.042882879 | 0.543 | 0.347 | 5.86E−67 | PHX48 | Gstm3 |
| 1100001G20Rik2 | 8.36E−71 | 0.349146589 | 0.982 | 0.98 | 2.08E−66 | PHX48 | 1100001G20Rik |
| Ang2 | 9.46E−68 | 0.554661583 | 0.773 | 0.72 | 2.35E−63 | PHX48 | Ang |
| Fga4 | 2.42E−66 | 0.410993472 | 0.992 | 0.998 | 6.01E−62 | PHX48 | Fga |
| C4bp1 | 6.90E−60 | 0.365491689 | 0.852 | 0.854 | 1.71E−55 | PHX48 | C4bp |
| Cfh2 | 1.21E−55 | 0.346063736 | 0.911 | 0.934 | 3.01E−51 | PHX48 | Cfh |
| Tmem176a1 | 2.65E−53 | 0.502002681 | 0.637 | 0.55 | 6.57E−49 | PHX48 | Tmem176a |
| Cp1 | 8.84E−53 | 0.346340984 | 0.952 | 0.967 | 2.20E−48 | PHX48 | Cp |
| Rpl412 | 2.41E−52 | 0.516910533 | 0.528 | 0.355 | 5.98E−48 | PHX48 | Rpl41 |
| Nnmt4 | 4.00E−52 | 0.431614865 | 0.825 | 0.799 | 9.92E−48 | PHX48 | Nnmt |
| Gstm2 | 5.13E−52 | 0.49759485 | 0.345 | 0.17 | 1.27E−47 | PHX48 | Gstm2 |
| Hsp90ab1 | 1.68E−51 | 0.300059266 | 0.923 | 0.951 | 4.16E−47 | PHX48 | Hsp90ab1 |
| Tmem176b1 | 6.17E−51 | 0.43901744 | 0.744 | 0.717 | 1.53E−46 | PHX48 | Tmem176b |
| Dbi1 | 1.25E−49 | 0.325825837 | 0.969 | 0.972 | 3.11E−45 | PHX48 | Dbi |
| Serpina104 | 4.43E−48 | 0.509521298 | 0.772 | 0.765 | 1.10E−43 | PHX48 | Serpina10 |
| Cyp7a13 | 5.38E−48 | 0.694022545 | 0.372 | 0.205 | 1.34E−43 | PHX48 | Cyp7a1 |
| Marco | 4.74E−47 | 0.26221596 | 0.106 | 0.023 | 1.18E−42 | PHX48 | Marco |
| Isyna1 | 9.14E−47 | 0.270858578 | 0.219 | 0.084 | 2.27E−42 | PHX48 | Isyna1 |
| Ccnd1 | 3.60E−45 | 0.325431422 | 0.218 | 0.084 | 8.93E−41 | PHX48 | Ccnd1 |
| Fgb4 | 1.38E−44 | 0.286867229 | 0.999 | 1 | 3.42E−40 | PHX48 | Fgb |
| Cd5l1 | 3.26E−43 | 0.339742048 | 0.199 | 0.071 | 8.09E−39 | PHX48 | Cd5l |
| Cd3021 | 5.51E−43 | 0.359660787 | 0.864 | 0.885 | 1.37E−38 | PHX48 | Cd302 |
| Ifitm22 | 1.71E−41 | 0.463721039 | 0.532 | 0.417 | 4.26E−37 | PHX48 | Ifitm2 |
| Fgg4 | 3.83E−38 | 0.293846068 | 0.996 | 0.996 | 9.52E−34 | PHX48 | Fgg |
| C1qb1 | 6.74E−30 | 0.27028615 | 0.163 | 0.064 | 1.67E−25 | PHX48 | C1qb |
| Alas12 | 1.49E−29 | 0.357004678 | 0.749 | 0.766 | 3.70E−25 | PHX48 | Alas1 |
| Cyp51 | 3.15E−26 | 0.428575287 | 0.67 | 0.678 | 7.82E−22 | PHX48 | Cyp51 |
| Rbp1 | 1.44E−25 | 0.312016246 | 0.264 | 0.148 | 3.58E−21 | PHX48 | Rbp1 |
| Gas6 | 4.77E−25 | 0.286153321 | 0.215 | 0.111 | 1.19E−20 | PHX48 | Gas6 |
| BC048546 | 5.45E−22 | 0.273479948 | 0.265 | 0.159 | 1.35E−17 | PHX48 | BC048546 |
| n-R5-8s14 | 1.33E−21 | 0.48615855 | 0.464 | 0.33 | 3.29E−17 | PHX48 | n-R5-8s1 |
| Dynll1 | 2.25E−21 | 0.302178838 | 0.283 | 0.181 | 5.60E−17 | PHX48 | Dynll1 |
| Gm12744 | 9.64E−21 | 0.289497387 | 0.231 | 0.134 | 2.39E−16 | PHX48 | Gm12744 |
| Acnat2 | 5.19E−20 | 0.272696647 | 0.252 | 0.154 | 1.29E−15 | PHX48 | Acnat2 |
| Krt183 | 2.39E−19 | 0.338485565 | 0.681 | 0.694 | 5.93E−15 | PHX48 | Krt18 |
| Qsox11 | 1.75E−18 | 0.277977801 | 0.752 | 0.807 | 4.34E−14 | PHX48 | Qsox1 |
| Tmsb4x1 | 2.19E−18 | 0.25903061 | 0.183 | 0.094 | 5.44E−14 | PHX48 | Tmsb4x |
| Fdps1 | 1.22E−16 | 0.363303117 | 0.492 | 0.438 | 3.04E−12 | PHX48 | Fdps |
| Oat4 | 1.69E−15 | 0.793230792 | 0.542 | 0.516 | 4.20E−11 | PHX48 | Oat |
| Lect22 | 6.99E−15 | 0.388301276 | 0.427 | 0.352 | 1.74E−10 | PHX48 | Lect2 |
| Apoc22 | 8.72E−15 | 0.290495283 | 0.803 | 0.837 | 2.17E−10 | PHX48 | Apoc2 |
| Ssr41 | 1.66E−14 | 0.305225519 | 0.646 | 0.667 | 4.12E−10 | PHX48 | Ssr4 |
| Cyp1a24 | 3.23E−12 | 0.325588327 | 0.708 | 0.707 | 8.03E−08 | PHX48 | Cyp1a2 |
| Rpl13a | 7.69E−10 | 0.263890271 | 0.766 | 0.831 | 1.91E−05 | PHX48 | Rpl13a |
| Mlec | 1.34E−07 | 0.324281741 | 0.436 | 0.417 | 0.003327101 | PHX48 | Mlec |
| Pmvk | 2.00E−07 | 0.254397796 | 0.359 | 0.322 | 0.004969378 | PHX48 | Pmvk |
| Lss | 7.67E−07 | 0.30143688 | 0.282 | 0.24 | 0.019046485 | PHX48 | Lss |
| Sc4mol2 | 3.84E−06 | 0.255232899 | 0.584 | 0.62 | 0.095387919 | PHX48 | Sc4mol |
| Smc4 | 4.95E−06 | 0.302249675 | 0.177 | 0.133 | 0.122794585 | PHX48 | Smc4 |
| Tuba1c | 1.47E−05 | 0.318082621 | 0.415 | 0.412 | 0.366264257 | PHX48 | Tuba1c |
| Glul2 | 4.53E−05 | 0.369104744 | 0.203 | 0.277 | 1 | PHX48 | Glul |
| Avpi1 | 0.000112837 | 0.313000373 | 0.409 | 0.416 | 1 | PHX48 | Avpi1 |
| Rps24 | 0.001118688 | 0.252072758 | 0.574 | 0.64 | 1 | PHX48 | Rps24 |
| Tubb5 | 0.004837713 | 0.325701472 | 0.304 | 0.294 | 1 | PHX48 | Tubb5 |
| Sqle | 0.007831353 | 0.262168232 | 0.379 | 0.392 | 1 | PHX48 | Sqle |
| Tubb4b | 0.009501966 | 0.323765623 | 0.347 | 0.352 | 1 | PHX48 | Tubb4b |
| Lcn26 | 0 | 1.118815683 | 0.925 | 0.331 | 0 | PHX120 | Lcn2 |
| Saa15 | 0 | 1.107521043 | 0.996 | 0.71 | 0 | PHX120 | Saa1 |
| Saa25 | 0 | 1.008333835 | 0.948 | 0.457 | 0 | PHX120 | Saa2 |
| Fgl14 | 0 | 0.874857754 | 0.993 | 0.864 | 0 | PHX120 | Fgl1 |
| Saa44 | 0 | 0.858219404 | 0.982 | 0.799 | 0 | PHX120 | Saa4 |
| Rnase43 | 0 | 0.818603379 | 0.993 | 0.926 | 0 | PHX120 | Rnase4 |
| Orm26 | 0 | 0.807371375 | 0.813 | 0.225 | 0 | PHX120 | Orm2 |
| Lrg13 | 0 | 0.788285793 | 0.982 | 0.844 | 0 | PHX120 | Lrg1 |
| Itih33 | 0 | 0.775773488 | 0.991 | 0.933 | 0 | PHX120 | Itih3 |
| Gm155644 | 0 | 0.753023538 | 1 | 0.973 | 0 | PHX120 | Gm15564 |
| C4b1 | 0 | 0.738227617 | 0.98 | 0.898 | 0 | PHX120 | C4b |
| C31 | 0 | 0.711130161 | 0.999 | 0.995 | 0 | PHX120 | C3 |
| Lars24 | 0 | 0.709570617 | 1 | 0.997 | 0 | PHX120 | Lars2 |
| Itih45 | 0 | 0.698397089 | 0.998 | 0.97 | 0 | PHX120 | Itih4 |
| Cfh3 | 0 | 0.669226929 | 0.991 | 0.924 | 0 | PHX120 | Cfh |
| Clu | 0 | 0.594745205 | 0.997 | 0.968 | 0 | PHX120 | Clu |
| Orm14 | 0 | 0.5647963 | 1 | 0.96 | 0 | PHX120 | Orm1 |
| Hp4 | 0 | 0.561007835 | 0.999 | 0.999 | 0 | PHX120 | Hp |
| Hpx4 | 1.09E−306 | 0.628252971 | 1 | 0.998 | 2.70E−302 | PHX120 | Hpx |
| Plg | 2.82E−295 | 0.468337901 | 0.997 | 0.973 | 7.01E−291 | PHX120 | Plg |
| Ly6e2 | 1.18E−292 | 0.899345798 | 0.821 | 0.483 | 2.92E−288 | PHX120 | Ly6e |
| Fga5 | 1.92E−290 | 0.521486803 | 0.999 | 0.997 | 4.76E−286 | PHX120 | Fga |
| Kng1 | 1.96E−279 | 0.383479985 | 0.999 | 0.99 | 4.88E−275 | PHX120 | Kng1 |
| Orm3 | 4.06E−270 | 0.509783113 | 0.47 | 0.115 | 1.01E−265 | PHX120 | Orm3 |
| mmu-mir-62362 | 4.62E−269 | 0.989356763 | 0.989 | 0.884 | 1.15E−264 | PHX120 | mmu-mir-6236 |
| Vtn3 | 1.71E−255 | 0.404732741 | 0.998 | 0.985 | 4.24E−251 | PHX120 | Vtn |
| Prtn31 | 4.90E−246 | 0.298794716 | 0.271 | 0.034 | 1.22E−241 | PHX120 | Prtn3 |
| Gm239351 | 5.60E−241 | 0.825787075 | 0.997 | 0.955 | 1.39E−236 | PHX120 | Gm23935 |
| Fn1 | 5.04E−228 | 0.640566195 | 0.968 | 0.884 | 1.25E−223 | PHX120 | Fn1 |
| Pzp | 9.55E−224 | 0.425391077 | 0.999 | 0.988 | 2.37E−219 | PHX120 | Pzp |
| Aldh1l1 | 1.54E−220 | 0.47413999 | 0.989 | 0.933 | 3.82E−216 | PHX120 | Aldh1l1 |
| Tmem176b2 | 9.29E−219 | 0.590562164 | 0.901 | 0.693 | 2.31E−214 | PHX120 | Tmem176b |
| Gm269245 | 1.34E−217 | 0.610417073 | 1 | 1 | 3.33E−213 | PHX120 | Gm26924 |
| Lrp11 | 4.26E−214 | 0.735936027 | 0.874 | 0.671 | 1.06E−209 | PHX120 | Lrp1 |
| Ambp1 | 4.27E−203 | 0.345542146 | 0.999 | 0.994 | 1.06E−198 | PHX120 | Ambp |
| Hc1 | 1.93E−192 | 0.502827072 | 0.974 | 0.914 | 4.79E−188 | PHX120 | Hc |
| Cp2 | 2.94E−183 | 0.408001782 | 0.995 | 0.961 | 7.30E−179 | PHX120 | Cp |
| Qsox12 | 1.53E−176 | 0.50367789 | 0.93 | 0.783 | 3.80E−172 | PHX120 | Qsox1 |
| Cyp2c704 | 2.83E−175 | 0.483246632 | 0.967 | 0.855 | 7.02E−171 | PHX120 | Cyp2c70 |
| Lpl | 2.92E−169 | 0.33278439 | 0.194 | 0.026 | 7.26E−165 | PHX120 | Lpl |
| Hrg3 | 2.98E−169 | 0.439374294 | 0.966 | 0.864 | 7.41E−165 | PHX120 | Hrg |
| Tmem176a2 | 9.05E−165 | 0.526399787 | 0.773 | 0.526 | 2.25E−160 | PHX120 | Tmem176a |
| Mpeg11 | 1.84E−162 | 0.455546657 | 0.435 | 0.15 | 4.57E−158 | PHX120 | Mpeg1 |
| Egfr4 | 2.19E−162 | 0.489699112 | 0.964 | 0.848 | 5.45E−158 | PHX120 | Egfr |
| Prg41 | 8.97E−161 | 0.593630978 | 0.643 | 0.347 | 2.23E−156 | PHX120 | Prg4 |
| Ifitm34 | 2.62E−158 | 0.454522399 | 0.944 | 0.828 | 6.51E−154 | PHX120 | Ifitm3 |
| Cml22 | 5.40E−158 | 0.446681218 | 0.953 | 0.871 | 1.34E−153 | PHX120 | Cml2 |
| Fgg5 | 5.05E−154 | 0.290840047 | 1 | 0.996 | 1.25E−149 | PHX120 | Fgg |
| Cd5l2 | 4.20E−149 | 0.397997645 | 0.263 | 0.056 | 1.04E−144 | PHX120 | Cd5l |
| Pck13 | 1.67E−147 | 0.360228951 | 0.999 | 0.976 | 4.14E−143 | PHX120 | Pck1 |
| Serping12 | 2.59E−145 | 0.404994618 | 0.964 | 0.891 | 6.42E−141 | PHX120 | Serping1 |
| Lbp | 1.52E−141 | 0.532670555 | 0.702 | 0.445 | 3.78E−137 | PHX120 | Lbp |
| Ang3 | 2.85E−139 | 0.532063038 | 0.874 | 0.703 | 7.08E−135 | PHX120 | Ang |
| Serpina3n4 | 1.58E−136 | 0.356286933 | 0.948 | 0.743 | 3.92E−132 | PHX120 | Serpina3n |
| Apcs4 | 7.41E−136 | 0.304899499 | 0.879 | 0.672 | 1.84E−131 | PHX120 | Apcs |
| Comt3 | 1.12E−135 | 0.356782039 | 0.987 | 0.931 | 2.78E−131 | PHX120 | Comt |
| Itih1 | 1.39E−120 | 0.385835499 | 0.936 | 0.855 | 3.45E−116 | PHX120 | Itih1 |
| Ctsb1 | 4.89E−120 | 0.402671607 | 0.927 | 0.839 | 1.21E−115 | PHX120 | Ctsb |
| Wfdc171 | 3.50E−119 | 0.26638224 | 0.195 | 0.038 | 8.70E−115 | PHX120 | Wfdc171 |
| Lyz21 | 4.94E−118 | 0.337893409 | 0.222 | 0.049 | 1.23E−113 | PHX120 | Lyz2 |
| Clec4f1 | 1.53E−117 | 0.360222876 | 0.282 | 0.078 | 3.79E−113 | PHX120 | Clec4f |
| Serpina105 | 3.87E−114 | 0.378970318 | 0.896 | 0.746 | 9.61E−110 | PHX120 | Serpina10 |
| H2-D11 | 4.89E−111 | 0.385804762 | 0.911 | 0.795 | 1.21E−106 | PHX120 | H2-D1 |
| C4bp2 | 8.95E−110 | 0.354982898 | 0.934 | 0.842 | 2.22E−105 | PHX120 | C4bp |
| Mug21 | 1.15E−102 | 0.431698452 | 0.881 | 0.773 | 2.85E−98 | PHX120 | Mug2 |
| C1qb2 | 2.58E−95 | 0.255909874 | 0.208 | 0.053 | 6.41E−91 | PHX120 | C1qb |
| Col18a11 | 4.56E−95 | 0.378250662 | 0.903 | 0.822 | 1.13E−90 | PHX120 | Col18a1 |
| Fetub | 8.03E−89 | 0.356300137 | 0.866 | 0.748 | 1.99E−84 | PHX120 | Fetub |
| C8a2 | 2.98E−88 | 0.331149118 | 0.964 | 0.892 | 7.39E−84 | PHX120 | C8a |
| n-R5-8s15 | 4.05E−87 | 0.323215543 | 0.578 | 0.308 | 1.00E−82 | PHX120 | n-R5-8s1 |
| Apob2 | 7.41E−84 | 0.310671508 | 0.997 | 0.988 | 1.84E−79 | PHX120 | Apob |
| Calr4 | 1.22E−83 | 0.273407417 | 0.961 | 0.903 | 3.04E−79 | PHX120 | Calr |
| Slc22a7 | 5.17E−82 | 0.325996046 | 0.405 | 0.2 | 1.28E−77 | PHX120 | Slc22a7 |
| Dap | 8.86E−79 | 0.347190068 | 0.842 | 0.718 | 2.20E−74 | PHX120 | Dap |
| Ldhd | 2.25E−78 | 0.347265823 | 0.766 | 0.627 | 5.59E−74 | PHX120 | Ldhd |
| Tat3 | 2.56E−78 | 0.300658052 | 0.988 | 0.934 | 6.35E−74 | PHX120 | Tat |
| H6pd | 1.14E−77 | 0.344480162 | 0.8 | 0.671 | 2.84E−73 | PHX120 | H6pd |
| Abcb112 | 9.04E−77 | 0.346201855 | 0.877 | 0.809 | 2.24E−72 | PHX120 | Abcb11 |
| Sephs24 | 4.17E−76 | 0.291789305 | 0.928 | 0.832 | 1.04E−71 | PHX120 | Sephs2 |
| Cfi | 2.06E−74 | 0.268077906 | 0.97 | 0.925 | 5.12E−70 | PHX120 | Cfi |
| Tmsb4x2 | 1.69E−73 | 0.264490332 | 0.24 | 0.082 | 4.20E−69 | PHX120 | Tmsb4x |
| Slc41a22 | 9.78E−72 | 0.296501328 | 0.446 | 0.235 | 2.43E−67 | PHX120 | Slc41a2 |
| Scd13 | 4.74E−71 | 0.324102197 | 0.996 | 0.939 | 1.18E−66 | PHX120 | Scd1 |
| Hamp21 | 8.82E−71 | 0.316130853 | 0.231 | 0.082 | 2.19E−66 | PHX120 | Hamp2 |
| Serpinf2 | 2.11E−70 | 0.270856246 | 0.944 | 0.873 | 5.23E−66 | PHX120 | Serpinf2 |
| Slc38a31 | 1.46E−69 | 0.280821059 | 0.936 | 0.874 | 3.64E−65 | PHX120 | Slc38a3 |
| Bsg1 | 2.16E−67 | 0.284697866 | 0.892 | 0.81 | 5.36E−63 | PHX120 | Bsg |
| Hspb83 | 1.11E−66 | 0.286758286 | 0.837 | 0.686 | 2.76E−62 | PHX120 | Hspb8 |
| Myh94 | 4.39E−65 | 0.334214402 | 0.652 | 0.467 | 1.09E−60 | PHX120 | Myh9 |
| Cpb21 | 5.07E−64 | 0.264151912 | 0.946 | 0.86 | 1.26E−59 | PHX120 | Cpb2 |
| Kng2 | 1.50E−63 | 0.281257218 | 0.833 | 0.732 | 3.73E−59 | PHX120 | Kng2 |
| Dhcr241 | 1.01E−62 | 0.289230487 | 0.92 | 0.854 | 2.52E−58 | PHX120 | Dhcr24 |
| Pcx | 3.13E−62 | 0.287981629 | 0.832 | 0.708 | 7.78E−58 | PHX120 | Pcx |
| Lgals91 | 7.62E−60 | 0.293933007 | 0.912 | 0.814 | 1.89E−55 | PHX120 | Lgals9 |
| Slc25a151 | 5.39E−57 | 0.261156509 | 0.896 | 0.795 | 1.34E−52 | PHX120 | Slc25a15 |
| Plxnb2 | 5.25E−56 | 0.338890275 | 0.54 | 0.372 | 1.30E−51 | PHX120 | Plxnb2 |
| Cpn21 | 2.28E−54 | 0.297606067 | 0.795 | 0.701 | 5.66E−50 | PHX120 | Cpn2 |
| Gne1 | 3.58E−54 | 0.324486089 | 0.673 | 0.528 | 8.89E−50 | PHX120 | Gne |
| Cyp3a13 | 3.22E−53 | 0.291486861 | 0.774 | 0.656 | 8.00E−49 | PHX120 | Cyp3a13 |
| Habp2 | 1.40E−52 | 0.287786829 | 0.61 | 0.47 | 3.47E−48 | PHX120 | Habp2 |
| F5 | 3.02E−50 | 0.30130754 | 0.84 | 0.773 | 7.49E−46 | PHX120 | F5 |
| Apon | 2.35E−48 | 0.264081329 | 0.811 | 0.703 | 5.84E−44 | PHX120 | Apon |
| Rgn3 | 2.74E−48 | 0.311282552 | 0.911 | 0.8 | 6.81E−44 | PHX120 | Rgn |
| Sardh1 | 7.55E−48 | 0.255777911 | 0.911 | 0.843 | 1.87E−43 | PHX120 | Sardh |
| Hsph1 | 4.81E−47 | 0.316405276 | 0.512 | 0.351 | 1.19E−42 | PHX120 | Hsph1 |
| Mbl22 | 1.07E−46 | 0.280531986 | 0.924 | 0.843 | 2.65E−42 | PHX120 | Mbl2 |
| Abat1 | 3.38E−45 | 0.272187232 | 0.808 | 0.721 | 8.39E−41 | PHX120 | Abat |
| Slc25a251 | 4.06E−45 | 0.275974133 | 0.866 | 0.767 | 1.01E−40 | PHX120 | Slc25a25 |
| Kynu1 | 5.14E−45 | 0.266831158 | 0.728 | 0.623 | 1.28E−40 | PHX120 | Kynu |
| Ugt1a1 | 3.78E−43 | 0.277601852 | 0.526 | 0.375 | 9.39E−39 | PHX120 | Ugt1a1 |
| Stat33 | 6.51E−43 | 0.267640194 | 0.602 | 0.458 | 1.62E−38 | PHX120 | Stat3 |
| Nbr11 | 1.83E−42 | 0.274748586 | 0.613 | 0.486 | 4.53E−38 | PHX120 | Nbr1 |
| Xdh | 2.51E−42 | 0.266596189 | 0.507 | 0.358 | 6.24E−38 | PHX120 | Xdh |
| Cyp2e14 | 3.41E−42 | 0.442070005 | 0.88 | 0.811 | 8.46E−38 | PHX120 | Cyp2e1 |
| Mafb1 | 1.11E−41 | 0.299984568 | 0.553 | 0.405 | 2.75E−37 | PHX120 | Mafb |
| 1300002K09Rik | 1.55E−41 | 0.274083731 | 0.673 | 0.562 | 3.85E−37 | PHX120 | 1300002K09Rik |
| Gulo1 | 2.72E−36 | 0.374663355 | 0.573 | 0.441 | 6.75E−32 | PHX120 | Gulo |
| Uroc11 | 3.16E−36 | 0.267582019 | 0.782 | 0.689 | 7.85E−32 | PHX120 | Uroc1 |
| Serpina6 | 1.33E−33 | 0.266334384 | 0.463 | 0.335 | 3.31E−29 | PHX120 | Serpina6 |
| Ifi272 | 8.57E−28 | 0.275927448 | 0.565 | 0.45 | 2.13E−23 | PHX120 | Ifi27 |
| Aldh1b1 | 2.35E−20 | 0.299248494 | 0.255 | 0.169 | 5.84E−16 | PHX120 | Aldh1b1 |
| Mup197 | 0 | 2.345313651 | 1 | 0.764 | 0 | UT | Mup19 |
| Mup176 | 0 | 2.220781802 | 1 | 0.873 | 0 | UT | Mup17 |
| Mup66 | 0 | 2.187799459 | 0.992 | 0.682 | 0 | UT | Mup6 |
| Mup97 | 0 | 2.1676942 | 0.976 | 0.446 | 0 | UT | Mup9 |
| Mup186 | 0 | 2.052181205 | 0.997 | 0.653 | 0 | UT | Mup18 |
| Mup57 | 0 | 1.497743401 | 0.839 | 0.437 | 0 | UT | Mup5 |
| Mup119 | 0 | 1.449872866 | 1 | 0.91 | 0 | UT | Mup11 |
| Ankrd555 | 0 | 1.403887011 | 0.696 | 0.35 | 0 | UT | Ankrd55 |
| Mup43 | 0 | 1.400873792 | 0.898 | 0.468 | 0 | UT | Mup4 |
| Mup156 | 0 | 1.2394731 | 0.617 | 0.171 | 0 | UT | Mup15 |
| Gm137757 | 0 | 1.138463541 | 0.761 | 0.242 | 0 | UT | Gm13775 |
| Mup166 | 0 | 1.034031769 | 0.934 | 0.638 | 0 | UT | Mup16 |
| Mup215 | 0 | 0.969567043 | 0.929 | 0.617 | 0 | UT | Mup21 |
| Mup86 | 0 | 0.774765659 | 0.373 | 0.093 | 0 | UT | Mup8 |
| Mup34 | 0 | 0.731785979 | 1 | 1 | 0 | UT | Mup3 |
| Mup126 | 0 | 0.633626671 | 0.86 | 0.598 | 0 | UT | Mup12 |
| Gsta34 | 0 | 0.524565372 | 0.956 | 0.955 | 0 | UT | Gsta3 |
| Cyb56 | 0 | 0.485470981 | 0.983 | 0.977 | 0 | UT | Cyb5 |
| Akr1c62 | 0 | 0.477461748 | 0.971 | 0.946 | 0 | UT | Akr1c6 |
| Mup203 | 7.83E−295 | 0.518904256 | 1 | 0.987 | 1.94E−290 | UT | Mup20 |
| Car34 | 3.61E−292 | 0.48897551 | 0.974 | 0.929 | 8.96E−288 | UT | Car3 |
| Acaa1b5 | 7.83E−288 | 0.432190174 | 0.969 | 0.86 | 1.94E−283 | UT | Acaa1b |
| Mup135 | 5.10E−281 | 0.730853769 | 0.546 | 0.266 | 1.27E−276 | UT | Mup13 |
| Fabp13 | 3.86E−269 | 0.324394469 | 1 | 0.995 | 9.59E−265 | UT | Fabp1 |
| Selenbp25 | 1.39E−266 | 0.467273497 | 0.937 | 0.817 | 3.44E−262 | UT | Selenbp2 |
| Elovl34 | 3.68E−264 | 0.539440847 | 0.827 | 0.632 | 9.14E−260 | UT | Elovl3 |
| Apoa25 | 3.93E−247 | 0.254295816 | 1 | 0.999 | 9.76E−243 | UT | Apoa2 |
| Serpina3c3 | 2.68E−243 | 0.508070161 | 0.385 | 0.133 | 6.66E−239 | UT | Serpina3c |
| Sparcl1 | 4.86E−229 | 0.253616986 | 0.136 | 0 | 1.21E−224 | UT | Sparcl1 |
| Cox4i14 | 1.79E−220 | 0.349876586 | 0.958 | 0.937 | 4.44E−216 | UT | Cox4i1 |
| Cyp2b105 | 4.10E−218 | 0.833168678 | 0.542 | 0.299 | 1.02E−213 | UT | Cyp2b10 |
| Ugt2b57 | 1.01E−213 | 0.319252422 | 0.959 | 0.937 | 2.51E−209 | UT | Ugt2b5 |
| Uqcrq3 | 3.26E−208 | 0.390101345 | 0.951 | 0.919 | 8.10E−204 | UT | Uqcrq |
| Hsd3b56 | 1.89E−182 | 0.552577049 | 0.587 | 0.371 | 4.70E−178 | UT | Hsd3b5 |
| Gm4076 | 6.76E−179 | 0.310732462 | 0.184 | 0.028 | 1.68E−174 | UT | Gm4076 |
| Rps141 | 1.79E−175 | 0.349394169 | 0.917 | 0.858 | 4.45E−171 | UT | Rsp14 |
| 2810007J24Rik2 | 1.71E−171 | 0.323006437 | 0.943 | 0.89 | 4.24E−167 | UT | 2810007J24Rik |
| Mgst12 | 3.86E−170 | 0.266779567 | 0.997 | 0.994 | 9.59E−166 | UT | Mgst1 |
| Igfbp22 | 2.57E−167 | 0.37970813 | 0.885 | 0.778 | 6.38E−163 | UT | Igfbp2 |
| Cox6c4 | 5.12E−167 | 0.427825866 | 0.884 | 0.8 | 1.27E−162 | UT | Cox6c |
| Mup24 | 2.49E−165 | 0.57964746 | 0.392 | 0.179 | 6.19E−161 | UT | Mup2 |
| Cyp2c296 | 3.84E−165 | 0.413790763 | 0.93 | 0.78 | 9.53E−161 | UT | Cyp2c29 |
| Serpina1a1 | 9.91E−159 | 0.27492561 | 0.991 | 0.971 | 2.46E−154 | UT | Serpina1a |
| Serpina1e3 | 1.04E−150 | 0.256934164 | 0.999 | 0.993 | 2.59E−146 | UT | Serpina1e |
| Inmt3 | 7.96E−146 | 0.419638578 | 0.884 | 0.855 | 1.98E−141 | UT | Inmt |
| Cyp2a56 | 7.33E−145 | 0.571080729 | 0.555 | 0.334 | 1.82E−140 | UT | Cyp2a5 |
| Hsd3b72 | 1.46E−143 | 0.388992153 | 0.798 | 0.715 | 3.63E−139 | UT | Hsd3b7 |
| Mup76 | 6.63E−141 | 0.580202644 | 0.998 | 0.958 | 1.65E−136 | UT | Mup7 |
| Cyp2c375 | 2.72E−138 | 0.393327144 | 0.608 | 0.371 | 6.74E−134 | UT | Cyp2c37 |
| Rps2 | 8.56E−125 | 0.396723019 | 0.757 | 0.624 | 2.13E−120 | UT | Rps2 |
| Ndufb112 | 1.77E−121 | 0.384829815 | 0.735 | 0.622 | 4.39E−117 | UT | Ndufb11 |
| Cyp2d93 | 2.14E−119 | 0.251733819 | 0.959 | 0.934 | 5.33E−115 | UT | Cyp2d9 |
| Prdx12 | 6.50E−118 | 0.275358573 | 0.927 | 0.903 | 1.61E−113 | UT | Prdx1 |
| Cyp2c505 | 1.48E−117 | 0.366535791 | 0.888 | 0.76 | 3.67E−113 | UT | Cyp2c50 |
| Oaz1 | 1.44E−112 | 0.371433095 | 0.75 | 0.664 | 3.58E−108 | UT | Oaz1 |
| Cfhr21 | 9.38E−112 | 0.359716521 | 0.868 | 0.852 | 2.33E−107 | UT | Cfhr2 |
| Tmem2052 | 4.61E−111 | 0.267844772 | 0.934 | 0.9 | 1.14E−106 | UT | Tmem205 |
| Aadac3 | 2.52E−109 | 0.370440569 | 0.86 | 0.844 | 6.26E−105 | UT | Aadac |
| Chchd22 | 5.87E−107 | 0.300651505 | 0.916 | 0.875 | 1.46E−102 | UT | Chchd2 |
| Cox7b2 | 6.77E−107 | 0.338982432 | 0.878 | 0.816 | 1.68E−102 | UT | Cox7b |
| Rps27l5 | 1.04E−102 | 0.30195065 | 0.849 | 0.759 | 2.57E−98 | UT | Rps27l |
| Ndufa43 | 1.43E−99 | 0.304406354 | 0.889 | 0.846 | 3.55E−95 | UT | Ndufa4 |
| Atp5f13 | 1.22E−98 | 0.297602439 | 0.869 | 0.847 | 3.03E−94 | UT | Atp5f1 |
| C8g3 | 2.17E−97 | 0.25042614 | 0.91 | 0.881 | 5.39E−93 | UT | C8g |
| Cyp2c545 | 7.72E−97 | 0.303236425 | 0.759 | 0.581 | 1.92E−92 | UT | Cyp2c54 |
| Cyp8b16 | 2.00E−90 | 0.267497706 | 0.745 | 0.611 | 4.96E−86 | UT | Cyp8b1 |
| Ugt2b12 | 4.18E−90 | 0.261161693 | 0.871 | 0.824 | 1.04E−85 | UT | Ugt2b1 |
| Ndufb63 | 3.74E−89 | 0.3153639 | 0.759 | 0.675 | 9.30E−85 | UT | Ndufb6 |
| Ahcy6 | 6.07E−84 | 0.250179307 | 0.916 | 0.847 | 1.51E−79 | UT | Ahcy |
| Adck33 | 4.25E−81 | 0.29387974 | 0.764 | 0.708 | 1.06E−76 | UT | Adck3 |
| Cyp1a25 | 3.41E−75 | 0.274168096 | 0.794 | 0.668 | 8.47E−71 | UT | Cyp1a2 |
| Gm205944 | 1.13E−70 | 0.40164744 | 0.294 | 0.164 | 2.80E−66 | UT | Gm20594 |
| Uqcrh2 | 1.78E−63 | 0.254384858 | 0.81 | 0.753 | 4.42E−59 | UT | Uqcrh |
| Cml12 | 3.50E−63 | 0.26577242 | 0.757 | 0.717 | 8.69E−59 | UT | Cml1 |
| Mup144 | 5.45E−63 | 0.506979284 | 0.616 | 0.509 | 1.35E−58 | UT | Mup14 |
| Ndufa62 | 2.82E−61 | 0.261286559 | 0.798 | 0.751 | 7.01E−57 | UT | Ndufa6 |
| Gm5096 | 1.47E−59 | 0.293645653 | 0.468 | 0.363 | 3.65E−55 | UT | Gm5096 |
| Nudt75 | 2.05E−59 | 0.340585682 | 0.83 | 0.796 | 5.08E−55 | UT | Nudt7 |
| Mup120 | 7.14E−58 | 0.561026142 | 0.583 | 0.483 | 1.77E−53 | UT | Mup1 |
| Rnf1252 | 2.04E−57 | 0.264315058 | 0.493 | 0.382 | 5.07E−53 | UT | Rnf125 |
| Fabp23 | 6.07E−56 | 0.283444626 | 0.622 | 0.522 | 1.51E−51 | UT | Fabp2 |
| Prdx4 | 6.69E−54 | 0.288185238 | 0.607 | 0.541 | 1.66E−49 | UT | Prdx4 |
| Rpl322 | 1.89E−51 | 0.263618639 | 0.757 | 0.692 | 4.68E−47 | UT | Rpl32 |
| Cox7a22 | 1.53E−50 | 0.252198292 | 0.784 | 0.736 | 3.79E−46 | UT | Cox7a2 |
| Ndufb41 | 3.21E−45 | 0.250792484 | 0.49 | 0.4 | 7.96E−41 | UT | Ndufb4 |
| Ndufs61 | 1.46E−43 | 0.258085508 | 0.724 | 0.672 | 3.62E−39 | UT | Ndufs6 |
| Gstp15 | 1.01E−42 | 0.281627196 | 0.822 | 0.759 | 2.50E−38 | UT | Gstp1 |
| Ndufc21 | 1.23E−32 | 0.252253197 | 0.579 | 0.544 | 3.05E−28 | UT | Ndufc2 |
| Ftl1 | 2.19E−24 | 0.319143693 | 0.409 | 0.358 | 5.43E−20 | UT | Ftl1 |
| Zfp6832 | 2.64E−17 | 0.277346647 | 0.116 | 0.071 | 6.55E−13 | UT | Zfp683 |

TABLE 1b
Treatment Markers Wilcox

| | _val | avg_logFC | pct.1 | pct.2 | p_val_adj | cluster | gene |
|---|---|---|---|---|---|---|---|
| Hbb-bs | 0 | 2.5859 | 0.537 | 0.038 | 0 | A6 | Hbb-bs |
| S100a9 | 0 | 1.34231 | 0.399 | 0.005 | 0 | A6 | S100a9 |
| Hmox1 | 0 | 1.33957 | 0.558 | 0.026 | 0 | A6 | Hmox1 |
| S100a8 | 0 | 1.12718 | 0.353 | 0.002 | 0 | A6 | S100a8 |
| Tnfrsf12a | 4.64E−274 | 1.10499 | 0.623 | 0.086 | 1.15E−269 | A6 | Tnfrsf12a |
| Ccl6 | 8.63E−274 | 0.78681 | 0.274 | 0.006 | 2.14E−269 | A6 | Ccl6 |
| Hilpda | 3.58E−254 | 0.78035 | 0.403 | 0.028 | 8.90E−250 | A6 | Hilpda |
| Mt2 | 4.30E−249 | 2.03768 | 0.938 | 0.405 | 1.07E−244 | A6 | Mt2 |
| Srgn | 3.95E−244 | 0.92893 | 0.284 | 0.01 | 9.80E−240 | A6 | Srgn |
| Mt1 | 5.85E−244 | 2.32039 | 0.981 | 0.602 | 1.45E−239 | A6 | Mt1 |
| Srxn1 | 1.13E−243 | 1.86421 | 0.821 | 0.239 | 2.80E−239 | A6 | Srxn1 |
| Ppl | 1.02E−233 | 0.86175 | 0.47 | 0.049 | 2.54E−229 | A6 | Ppl |
| Slc16a6 | 7.70E−208 | 0.91397 | 0.47 | 0.058 | 1.91E−203 | A6 | Slc16a6 |
| Gclc | 1.87E−200 | 1.80238 | 0.962 | 0.764 | 4.64E−196 | A6 | Gclc |
| Cdkn1a | 1.22E−180 | 1.25188 | 0.702 | 0.195 | 3.04E−176 | A6 | Cdkn1a |
| Plin2 | 1.48E−175 | 1.45429 | 0.943 | 0.742 | 3.67E−171 | A6 | Plin2 |
| Tubb6 | 1.52E−170 | 0.57153 | 0.368 | 0.04 | 3.78E−166 | A6 | Tubb6 |
| Actb | 9.38E−169 | 1.0516 | 0.979 | 0.893 | 2.33E−164 | A6 | Actb |
| Clec4f | 3.46E−163 | 1.12406 | 0.339 | 0.034 | 8.59E−159 | A6 | Clec4f |
| Chka | 5.96E−156 | 1.40324 | 0.716 | 0.24 | 1.48E−151 | A6 | Chka |
| Txnrd1 | 7.87E−143 | 1.37471 | 0.838 | 0.516 | 1.95E−138 | A6 | Txnrd1 |
| Il1r1 | 5.63E−137 | 1.18867 | 0.683 | 0.247 | 1.40E−132 | A6 | Il1r1 |
| Krt18 | 3.50E−128 | 1.22726 | 0.864 | 0.653 | 8.70E−124 | A6 | Krt18 |
| Cidec | 1.77E−123 | 0.30674 | 0.253 | 0.024 | 4.40E−119 | A6 | Cidec |
| Krt8 | 8.28E−122 | 1.27348 | 0.826 | 0.564 | 2.06E−117 | A6 | Krt8 |
| Klf6 | 8.56E−120 | 0.70668 | 0.382 | 0.066 | 2.13E−115 | A6 | Klf6 |
| Ets2 | 2.20E−109 | 0.68055 | 0.415 | 0.087 | 5.47E−105 | A6 | Ets2 |
| 2010003K11Rik | 3.63E−105 | 0.5729 | 0.396 | 0.081 | 9.02E−101 | A6 | 2010003K11Rik |
| Tmsb4x | 6.61E−105 | 0.7911 | 0.332 | 0.055 | 1.64E−100 | A6 | Tmsb4x |
| Cers6 | 1.33E−104 | 0.50708 | 0.332 | 0.055 | 3.29E−100 | A6 | Cers6 |
| Plscr1 | 2.32E−103 | 0.4497 | 0.329 | 0.055 | 5.75E−99 | A6 | Plscr1 |
| Rcan1 | 1.90E−94 | 0.70098 | 0.425 | 0.105 | 4.72E−90 | A6 | Rcan1 |
| Gm26924 | 2.05E−93 | 0.72692 | 1 | 1 | 5.10E−89 | A6 | Gm26924 |
| Myh9 | 2.39E−90 | 1.02483 | 0.718 | 0.401 | 5.92E−86 | A6 | Myh9 |
| Fgg | 1.41E−82 | 0.47508 | 0.986 | 0.995 | 3.50E−78 | A6 | Fgg |
| Serpina3m | 2.54E−82 | 0.79319 | 0.85 | 0.772 | 6.30E−78 | A6 | Serpina3m |
| C230081A13Rik | 5.32E−82 | 0.41842 | 0.279 | 0.049 | 1.32E−77 | A6 | C230081A13Rik |
| Lrrc58 | 6.20E−82 | 0.93964 | 0.711 | 0.429 | 1.54E−77 | A6 | Lrrc58 |
| Fga | 8.43E−81 | 0.501 | 0.986 | 0.997 | 2.09E−76 | A6 | Fga |
| Apoa4 | 3.02E−80 | 1.16604 | 0.721 | 0.434 | 7.51E−76 | A6 | Apoa4 |
| Rell1 | 5.69E−79 | 0.57926 | 0.375 | 0.095 | 1.41E−74 | A6 | Rell1 |
| Bcl2l1 | 9.17E−79 | 0.7415 | 0.501 | 0.182 | 2.28E−74 | A6 | Bcl2l1 |
| Fgb | 2.77E−76 | 0.47741 | 0.995 | 1 | 6.88E−72 | A6 | Fgb |
| Lars2 | 4.16E−75 | 0.81426 | 1 | 0.995 | 1.03E−70 | A6 | Lars2 |
| D10Wsu102e | 3.45E−74 | 0.56983 | 0.37 | 0.097 | 8.56E−70 | A6 | D10Wsu102e |
| Gsta1 | 2.23E−70 | 0.40892 | 0.32 | 0.073 | 5.53E−66 | A6 | Gsta1 |
| Slc39a14 | 6.11E−70 | 0.91609 | 0.678 | 0.428 | 1.52E−65 | A6 | Slc39a14 |
| Mrap | 9.06E−70 | 0.86148 | 0.606 | 0.323 | 2.25E−65 | A6 | Mrap |
| Gm15564 | 3.80E−66 | 0.78611 | 0.983 | 0.967 | 9.43E−62 | A6 | Gm15564 |
| 1700017B05Rik | 1.21E−62 | 0.32766 | 0.255 | 0.052 | 3.00E−58 | A6 | 1700017B05Rik |
| Igfbp1 | 9.02E−61 | 1.0899 | 0.792 | 0.702 | 2.24E−56 | A6 | Igfbp1 |
| Sdc4 | 7.44E−59 | 0.56725 | 0.909 | 0.892 | 1.85E−54 | A6 | Sdc4 |
| Cldn14 | 1.21E−58 | 0.3695 | 0.32 | 0.085 | 3.00E−54 | A6 | Cldn14 |
| Alb | 4.24E−58 | 0.37487 | 1 | 1 | 1.05E−53 | A6 | Alb |
| Sat1 | 3.50E−57 | 0.68873 | 0.47 | 0.193 | 8.68E−53 | A6 | Sat1 |
| Hnf4a | 7.04E−57 | 0.69625 | 0.835 | 0.805 | 1.75E−52 | A6 | Hnf4a |
| Sqstm1 | 1.54E−56 | 0.69921 | 0.859 | 0.828 | 3.83E−52 | A6 | Sqstm1 |
| Fam134b | 3.22E−56 | 0.81523 | 0.678 | 0.482 | 8.00E−52 | A6 | Fam134b |
| Nop58 | 2.76E−54 | 0.6343 | 0.492 | 0.216 | 6.86E−50 | A6 | Nop58 |
| Efhd2 | 4.11E−53 | 0.57664 | 0.468 | 0.197 | 1.02E−48 | A6 | Efhd2 |
| mmu-mir-6240 | 5.36E−53 | 1.03161 | 0.413 | 0.149 | 1.33E−48 | A6 | mmu-mir-6240 |
| Trp53inp1 | 9.22E−52 | 0.88291 | 0.637 | 0.398 | 2.29E−47 | A6 | Trp53inp1 |
| Epas1 | 5.89E−51 | 0.71377 | 0.604 | 0.373 | 1.46E−46 | A6 | Epas1 |
| Apob | 8.74E−50 | 0.48025 | 0.99 | 0.989 | 2.17E−45 | A6 | Apob |
| Gcnt2 | 4.90E−49 | 0.50385 | 0.372 | 0.131 | 1.22E−44 | A6 | Gcnt2 |
| Eif1a | 5.09E−49 | 0.7228 | 0.599 | 0.373 | 1.26E−44 | A6 | Eif1a |
| Insig2 | 1.13E−47 | 0.60809 | 0.854 | 0.83 | 2.80E−43 | A6 | Insig2 |
| Serpina3n | 2.16E−47 | 0.66415 | 0.747 | 0.658 | 5.38E−43 | A6 | Serpina3n |
| Btg2 | 1.37E−46 | 0.76689 | 0.549 | 0.303 | 3.40E−42 | A6 | Btg2 |
| Agpat9 | 3.40E−46 | 0.30485 | 0.277 | 0.077 | 8.45E−42 | A6 | Agpat9 |
| Tra2a | 1.23E−45 | 0.70805 | 0.337 | 0.114 | 3.05E−41 | A6 | Tra2a |
| Creb3l2 | 1.45E−44 | 0.39712 | 0.329 | 0.11 | 3.61E−40 | A6 | Creb3l2 |
| Stat3 | 1.26E−43 | 0.652 | 0.604 | 0.387 | 3.12E−39 | A6 | Stat3 |
| Picalm | 3.98E−43 | 0.67656 | 0.647 | 0.452 | 9.89E−39 | A6 | Picalm |
| Tubb2a | 8.70E−43 | 0.78599 | 0.618 | 0.443 | 2.16E−38 | A6 | Tubb2a |
| Mat1a | 9.42E−43 | 0.38887 | 0.986 | 0.973 | 2.34E−38 | A6 | Mat1a |
| Malat1 | 3.57E−42 | 0.98537 | 0.871 | 0.75 | 8.87E−38 | A6 | Malat1 |
| Cxadr | 1.77E−41 | 0.65684 | 0.606 | 0.408 | 4.40E−37 | A6 | Cxadr |
| Myo1e | 5.97E−41 | 0.57864 | 0.475 | 0.237 | 1.48E−36 | A6 | Myo1e |
| Bach1 | 8.93E−41 | 0.76858 | 0.461 | 0.236 | 2.22E−36 | A6 | Bach1 |
| Eif5 | 1.03E−40 | 0.48089 | 0.816 | 0.786 | 2.56E−36 | A6 | Eif5 |
| Tiparp | 2.49E−40 | 0.52481 | 0.346 | 0.131 | 6.18E−36 | A6 | Tiparp |
| Cltc | 7.70E−40 | 0.68523 | 0.661 | 0.494 | 1.91E−35 | A6 | Cltc |
| Ddx21 | 1.55E−39 | 0.65593 | 0.535 | 0.316 | 3.84E−35 | A6 | Ddx21 |
| Mdn1 | 2.43E−39 | 0.30793 | 0.251 | 0.072 | 6.04E−35 | A6 | Mdn1 |
| Heatr1 | 4.55E−39 | 0.38146 | 0.291 | 0.095 | 1.13E−34 | A6 | Heatr1 |
| H3f3b | 4.97E−39 | 0.71838 | 0.613 | 0.443 | 1.23E−34 | A6 | H3f3b |
| Jmjd1c | 3.17E−38 | 0.59632 | 0.47 | 0.239 | 7.87E−34 | A6 | Jmjd1c |
| Dot1l | 4.16E−38 | 0.38527 | 0.282 | 0.092 | 1.03E−33 | A6 | Dot1l |
| Ctsl | 6.53E−38 | 0.51275 | 0.878 | 0.88 | 1.62E−33 | A6 | Ctsl |
| Slc10a2 | 1.11E−37 | 0.27045 | 0.255 | 0.077 | 2.75E−33 | A6 | Slc10a2 |
| B4galt5 | 1.26E−37 | 0.42388 | 0.332 | 0.126 | 3.12E−33 | A6 | B4galt5 |
| Rock2 | 2.45E−37 | 0.55518 | 0.425 | 0.199 | 6.08E−33 | A6 | Rock2 |
| Dgkd | 6.05E−37 | 0.35842 | 0.282 | 0.094 | 1.50E−32 | A6 | Dgkd |
| Lcn2 | 1.67E−36 | 0.38757 | 0.301 | 0.106 | 4.14E−32 | A6 | Lcn2 |
| Hspb8 | 1.95E−36 | 0.60892 | 0.709 | 0.633 | 4.84E−32 | A6 | Hspb8 |
| Ugdh | 3.59E−36 | 0.60855 | 0.721 | 0.647 | 8.92E−32 | A6 | Ugdh |
| Ppan | 9.70E−36 | 0.27669 | 0.255 | 0.079 | 2.41E−31 | A6 | Ppan |
| Xbp1 | 1.86E−35 | 0.56939 | 0.692 | 0.611 | 4.62E−31 | A6 | Xbp1 |
| Ddx5 | 2.16E−35 | 0.53966 | 0.685 | 0.543 | 5.37E−31 | A6 | Ddx5 |
| Tnfrsf1a | 2.76E−35 | 0.56814 | 0.508 | 0.305 | 6.86E−31 | A6 | Tnfrsf1a |
| Tsr1 | 5.76E−35 | 0.33922 | 0.341 | 0.135 | 1.43E−30 | A6 | Tsr1 |
| Itih3 | 6.08E−35 | 0.40422 | 0.907 | 0.913 | 1.51E−30 | A6 | Itih3 |
| Ddx3x | 6.10E−35 | 0.56117 | 0.692 | 0.592 | 1.51E−30 | A6 | Ddx3x |
| St3gal1 | 9.22E−35 | 0.51699 | 0.473 | 0.262 | 2.29E−30 | A6 | St3gal1 |
| Eaf1 | 1.20E−34 | 0.50717 | 0.427 | 0.212 | 2.98E−30 | A6 | Eaf1 |
| Hsd17b13 | 2.16E−34 | 0.48038 | 0.864 | 0.761 | 5.37E−30 | A6 | Hsd17b13 |
| Abca1 | 1.44E−33 | 0.65393 | 0.604 | 0.438 | 3.58E−29 | A6 | Abca1 |
| Hif1a | 4.80E−33 | 0.48757 | 0.427 | 0.213 | 1.19E−28 | A6 | Hif1a |
| Tmem87b | 1.08E−32 | 0.40541 | 0.351 | 0.151 | 2.68E−28 | A6 | Tmem87b |
| Fkbp5 | 1.20E−32 | 0.45674 | 0.415 | 0.197 | 2.97E−28 | A6 | Fkbp5 |
| C3 | 2.78E−32 | 0.27846 | 0.993 | 0.993 | 6.90E−28 | A6 | C3 |
| Spred1 | 3.89E−31 | 0.35201 | 0.308 | 0.122 | 9.65E−27 | A6 | Spred1 |
| Eef2 | 8.96E−31 | 0.40594 | 0.9 | 0.899 | 2.23E−26 | A6 | Eef2 |
| Gnl3 | 1.03E−30 | 0.49018 | 0.501 | 0.3 | 2.56E−26 | A6 | Gnl3 |
| Itih2 | 1.23E−30 | 0.39267 | 0.893 | 0.914 | 3.06E−26 | A6 | Itih2 |
| Stk40 | 4.64E−30 | 0.493 | 0.382 | 0.184 | 1.15E−25 | A6 | Stk40 |
| Tjp2 | 4.42E−29 | 0.39613 | 0.332 | 0.147 | 1.10E−24 | A6 | Tjp2 |
| Lyst | 1.82E−28 | 0.37173 | 0.31 | 0.127 | 4.52E−24 | A6 | Lyst |
| Errfi1 | 2.21E−28 | 0.34209 | 0.943 | 0.931 | 5.50E−24 | A6 | Errfi1 |
| Chic2 | 3.18E−28 | 0.3964 | 0.37 | 0.181 | 7.89E−24 | A6 | Chic2 |
| Rad54l2 | 3.33E−28 | 0.36421 | 0.325 | 0.139 | 8.26E−24 | A6 | Rad54l2 |
| Mfsd2a | 4.59E−28 | 0.46105 | 0.465 | 0.263 | 1.14E−23 | A6 | Mfsd2a |
| Sdad1 | 1.02E−27 | 0.29694 | 0.289 | 0.115 | 2.54E−23 | A6 | Sdad1 |
| Bel3 | 2.52E−27 | 0.27406 | 0.277 | 0.109 | 6.26E−23 | A6 | Bel3 |
| Neat1 | 3.08E−27 | 0.65541 | 0.31 | 0.135 | 7.66E−23 | A6 | Neat1 |
| Asl | 4.46E−27 | 0.41345 | 0.826 | 0.822 | 1.11E−22 | A6 | Asl |
| Tcerg1 | 5.22E−27 | 0.34681 | 0.305 | 0.132 | 1.30E−22 | A6 | Tcerg1 |
| Tacc2 | 5.92E−27 | 0.31276 | 0.289 | 0.116 | 1.47E−22 | A6 | Tacc2 |
| Gpt2 | 9.88E−27 | 0.46947 | 0.764 | 0.767 | 2.45E−22 | A6 | Gpt2 |
| Abhd2 | 2.12E−26 | 0.41489 | 0.37 | 0.181 | 5.25E−22 | A6 | Abhd2 |
| Cyp4a14 | 2.69E−26 | 0.33337 | 0.776 | 0.554 | 6.69E−22 | A6 | Cyp4a14 |
| Ptpn1 | 4.30E−26 | 0.38653 | 0.353 | 0.165 | 1.07E−21 | A6 | Ptpn1 |
| Fndc3b | 9.70E−26 | 0.34961 | 0.315 | 0.14 | 2.41E−21 | A6 | Fndc3b |
| Tsku | 3.63E−25 | 0.33318 | 0.327 | 0.148 | 9.01E−21 | A6 | Tsku |
| Nolc1 | 6.52E−25 | 0.44449 | 0.363 | 0.183 | 1.62E−20 | A6 | Nolc1 |
| Jak1 | 1.07E−24 | 0.54371 | 0.554 | 0.415 | 2.67E−20 | A6 | Jak1 |
| Tsc22d2 | 2.31E−24 | 0.36291 | 0.353 | 0.175 | 5.73E−20 | A6 | Tsc22d2 |
| Btg1 | 2.53E−24 | 0.39842 | 0.356 | 0.177 | 6.29E−20 | A6 | Btg1 |
| Son | 2.57E−24 | 0.4555 | 0.601 | 0.439 | 6.39E−20 | A6 | Son |
| Lpin2 | 3.86E−24 | 0.51652 | 0.745 | 0.687 | 9.59E−20 | A6 | Lpin2 |
| Iigp1 | 5.96E−24 | 0.64313 | 0.78 | 0.805 | 1.48E−19 | A6 | Iigp1 |
| St3gal5 | 6.19E−24 | 0.43579 | 0.706 | 0.581 | 1.54E−19 | A6 | St3gal5 |
| n-R5-8s1 | 1.02E−23 | 0.40051 | 0.308 | 0.135 | 2.53E−19 | A6 | n-R5-8s1 |
| Eif4g2 | 1.15E−23 | 0.41256 | 0.757 | 0.748 | 2.86E−19 | A6 | Eif4g2 |
| Gjb2 | 2.31E−23 | 0.50382 | 0.678 | 0.644 | 5.73E−19 | A6 | Gjb2 |
| Dusp16 | 1.08E−22 | 0.42732 | 0.372 | 0.203 | 2.68E−18 | A6 | Dusp16 |
| Pfkfb2 | 1.30E−22 | 0.26939 | 0.26 | 0.108 | 3.23E−18 | A6 | Pfkfb2 |
| Tars | 1.56E−22 | 0.47178 | 0.666 | 0.587 | 3.87E−18 | A6 | Tars |
| Rora | 2.50E−22 | 0.54096 | 0.549 | 0.433 | 6.22E−18 | A6 | Rora |
| Plec | 3.98E−22 | 0.41883 | 0.422 | 0.244 | 9.88E−18 | A6 | Plec |
| Gm26917 | 6.93E−22 | 0.42391 | 0.382 | 0.204 | 1.72E−17 | A6 | Gm26917 |
| Mpeg1 | 1.75E−21 | 0.46679 | 0.263 | 0.113 | 4.34E−17 | A6 | Mpeg1 |
| Mat2a | 3.17E−21 | 0.47583 | 0.487 | 0.332 | 7.86E−17 | A6 | Mat2a |
| Gtpbp4 | 3.37E−21 | 0.46234 | 0.477 | 0.317 | 8.37E−17 | A6 | Gtpbp4 |
| Ranbp2 | 4.52E−21 | 0.50743 | 0.566 | 0.435 | 1.12E−16 | A6 | Ranbp2 |
| Map2k3 | 6.02E−21 | 0.41497 | 0.427 | 0.262 | 1.50E−16 | A6 | Map2k3 |
| Mcl1 | 6.44E−21 | 0.5249 | 0.573 | 0.434 | 1.60E−16 | A6 | Mcl1 |
| Slc20a1 | 1.39E−20 | 0.32604 | 0.284 | 0.131 | 3.44E−16 | A6 | Slc20a1 |
| Cars | 1.86E−20 | 0.30674 | 0.329 | 0.166 | 4.61E−16 | A6 | Cars |
| Ywhag | 3.03E−20 | 0.51318 | 0.518 | 0.392 | 7.52E−16 | A6 | Ywhag |
| Retsat | 4.73E−20 | 0.29183 | 0.845 | 0.85 | 1.18E−15 | A6 | Retsat |
| Etf1 | 4.99E−20 | 0.45256 | 0.606 | 0.499 | 1.24E−15 | A6 | Etf1 |
| Epb4.1 | 6.09E−20 | 0.46777 | 0.537 | 0.403 | 1.51E−15 | A6 | Epb4.1 |
| Myl12a | 8.41E−20 | 0.40288 | 0.461 | 0.306 | 2.09E−15 | A6 | Myl12a |
| Dusp6 | 3.85E−19 | 0.50868 | 0.482 | 0.335 | 9.57E−15 | A6 | Dusp6 |
| Gm20594 | 7.21E−19 | 0.4405 | 0.442 | 0.259 | 1.79E−14 | A6 | Gm20594 |
| Lrg1 | 7.52E−19 | 0.3324 | 0.783 | 0.797 | 1.87E−14 | A6 | Lrg1 |
| Dkc1 | 8.07E−19 | 0.30545 | 0.315 | 0.162 | 2.00E−14 | A6 | Dkc1 |
| App | 1.30E−18 | 0.33806 | 0.315 | 0.162 | 3.23E−14 | A6 | App |
| Rnd3 | 1.41E−18 | 0.32675 | 0.284 | 0.14 | 3.50E−14 | A6 | Rnd3 |
| Ncl | 1.56E−18 | 0.38819 | 0.69 | 0.639 | 3.88E−14 | A6 | Ncl |
| Tatdn2 | 1.76E−18 | 0.32476 | 0.334 | 0.176 | 4.36E−14 | A6 | Tatdn2 |
| Fam107b | 2.12E−18 | 0.41622 | 0.449 | 0.307 | 5.26E−14 | A6 | Fam107b |
| Tns1 | 3.23E−18 | 0.40012 | 0.403 | 0.254 | 8.02E−14 | A6 | Tns1 |
| Phc2 | 3.78E−18 | 0.29899 | 0.284 | 0.141 | 9.39E−14 | A6 | Phc2 |
| Tbc1d15 | 3.97E−18 | 0.43222 | 0.461 | 0.319 | 9.86E−14 | A6 | Tbc1d15 |
| Dst | 3.98E−18 | 0.44734 | 0.43 | 0.276 | 9.87E−14 | A6 | Dst |
| Lmo4 | 4.29E−18 | 0.29625 | 0.308 | 0.162 | 1.06E−13 | A6 | Lmo4 |
| Egr1 | 5.65E−18 | 0.53704 | 0.589 | 0.481 | 1.40E−13 | A6 | Egr1 |
| Hnrnpdl | 8.07E−18 | 0.39933 | 0.406 | 0.254 | 2.00E−13 | A6 | Hnrnpdl |
| Aff4 | 1.70E−17 | 0.53634 | 0.537 | 0.423 | 4.23E−13 | A6 | Aff4 |
| Gnl2 | 1.83E−17 | 0.35803 | 0.384 | 0.235 | 4.55E−13 | A6 | Gnl2 |
| Lrrfip1 | 2.21E−17 | 0.41083 | 0.37 | 0.221 | 5.49E−13 | A6 | Lrrfip1 |
| Aass | 2.35E−17 | 0.40708 | 0.747 | 0.763 | 5.83E−13 | A6 | Aass |
| Kif5b | 8.07E−17 | 0.45223 | 0.494 | 0.379 | 2.00E−12 | A6 | Kif5b |
| Wbp1l | 1.01E−16 | 0.3897 | 0.697 | 0.683 | 2.52E−12 | A6 | Wbp1l |
| Stom | 1.12E−16 | 0.29996 | 0.329 | 0.184 | 2.77E−12 | A6 | Stom |
| Arhgef12 | 1.63E−16 | 0.48561 | 0.578 | 0.494 | 4.05E−12 | A6 | Arhgef12 |
| Pxk | 4.69E−16 | 0.26523 | 0.303 | 0.161 | 1.16E−11 | A6 | Pxk |
| Prrc2c | 6.21E−16 | 0.39059 | 0.415 | 0.266 | 1.54E−11 | A6 | Prrc2c |
| Sds | 6.91E−16 | 0.35155 | 0.73 | 0.632 | 1.72E−11 | A6 | Sds |
| Mybbp1a | 7.16E−16 | 0.4018 | 0.396 | 0.263 | 1.78E−11 | A6 | Mybbp1a |
| Wdr43 | 8.36E−16 | 0.30829 | 0.353 | 0.208 | 2.08E−11 | A6 | Wdr43 |
| Mmp14 | 9.37E−16 | 0.25714 | 0.253 | 0.125 | 2.33E−11 | A6 | Mmp14 |
| Ptpn2 | 9.68E−16 | 0.27096 | 0.255 | 0.124 | 2.40E−11 | A6 | Ptpn2 |
| Mak16 | 1.01E−15 | 0.28973 | 0.267 | 0.134 | 2.51E−11 | A6 | Mak16 |
| Plk3 | 1.73E−15 | 0.41583 | 0.413 | 0.269 | 4.29E−11 | A6 | Plk3 |
| Tm4sf4 | 2.46E−15 | 0.40056 | 0.484 | 0.363 | 6.11E−11 | A6 | Tm4sf4 |
| Cyp39a1 | 4.02E−15 | 0.29958 | 0.305 | 0.171 | 9.99E−11 | A6 | Cyp39a1 |
| Nedd4l | 4.88E−15 | 0.26354 | 0.332 | 0.189 | 1.21E−10 | A6 | Nedd4l |
| Ptpn12 | 6.24E−15 | 0.30933 | 0.263 | 0.135 | 1.55E−10 | A6 | Ptpn12 |
| Map3k5 | 6.60E−15 | 0.31306 | 0.346 | 0.206 | 1.64E−10 | A6 | Map3k5 |
| Nars | 7.95E−15 | 0.36869 | 0.606 | 0.539 | 1.97E−10 | A6 | Nars |
| Ptp4a1 | 9.30E−15 | 0.25171 | 0.334 | 0.192 | 2.31E−10 | A6 | Ptp4a1 |
| Gm24245 | 1.17E−14 | 0.49843 | 0.422 | 0.269 | 2.89E−10 | A6 | Gm24245 |
| Ifrd1 | 1.21E−14 | 0.47648 | 0.365 | 0.226 | 3.00E−10 | A6 | Ifrd1 |
| AA474408 | 1.38E−14 | 0.26642 | 0.274 | 0.142 | 3.42E−10 | A6 | AA474408 |
| Zyx | 2.09E−14 | 0.27986 | 0.263 | 0.137 | 5.20E−10 | A6 | Zyx |
| Smg1 | 2.32E−14 | 0.30447 | 0.265 | 0.141 | 5.76E−10 | A6 | Smg1 |
| Man2a1 | 3.27E−14 | 0.34393 | 0.671 | 0.659 | 8.13E−10 | A6 | Man2a1 |
| Lrrfip2 | 3.29E−14 | 0.35649 | 0.384 | 0.247 | 8.16E−10 | A6 | Lrrfip2 |
| Ppp1r12a | 3.38E−14 | 0.32516 | 0.317 | 0.182 | 8.39E−10 | A6 | Ppp1r12a |
| Chd1 | 4.71E−14 | 0.31413 | 0.377 | 0.243 | 1.17E−09 | A6 | Chd1 |
| Lpgat1 | 5.37E−14 | 0.4435 | 0.618 | 0.593 | 1.33E−09 | A6 | Lpgat1 |
| Palld | 6.63E−14 | 0.30837 | 0.353 | 0.218 | 1.65E−09 | A6 | Palld |
| H2-D1 | 6.78E−14 | 0.29863 | 0.766 | 0.781 | 1.68E−09 | A6 | H2-D1 |
| Grn | 7.22E−14 | 0.37381 | 0.582 | 0.499 | 1.79E−09 | A6 | Grn |
| Fus | 1.11E−13 | 0.33882 | 0.394 | 0.267 | 2.76E−09 | A6 | Fus |
| Litaf | 1.26E−13 | 0.35117 | 0.451 | 0.34 | 3.13E−09 | A6 | Litaf |
| Mki67ip | 1.50E−13 | 0.31217 | 0.344 | 0.212 | 3.73E−09 | A6 | Mki67ip |
| Lcp1 | 2.39E−13 | 0.4649 | 0.492 | 0.365 | 5.94E−09 | A6 | Lcp1 |
| mt-Rnr1 | 2.60E−13 | 0.39009 | 1 | 1 | 6.45E−09 | A6 | mt-Rnr1 |
| Ctsb | 2.79E−13 | 0.46911 | 0.804 | 0.82 | 6.94E−09 | A6 | Ctsb |
| Sptbn1 | 3.21E−13 | 0.37622 | 0.439 | 0.325 | 7.98E−09 | A6 | Sptbn1 |
| Sfpq | 5.31E−13 | 0.34485 | 0.406 | 0.277 | 1.32E−08 | A6 | sfpq |
| Tgm2 | 5.71E−13 | 0.36214 | 0.446 | 0.314 | 1.42E−08 | A6 | Tgm2 |
| Eif4g1 | 1.45E−12 | 0.31441 | 0.723 | 0.719 | 3.60E−08 | A6 | Eif4g1 |
| Fn1 | 2.32E−12 | 0.2852 | 0.847 | 0.866 | 5.76E−08 | A6 | Fn1 |
| Purb | 2.57E−12 | 0.31082 | 0.341 | 0.219 | 6.37E−08 | A6 | Purb |
| Phlda1 | 2.84E−12 | 0.47234 | 0.494 | 0.429 | 7.05E−08 | A6 | Phlda1 |
| Atp2a2 | 3.95E−12 | 0.36462 | 0.616 | 0.575 | 9.81E−08 | A6 | Atp2a2 |
| Etnppl | 6.99E−12 | 0.30441 | 0.706 | 0.62 | 1.74E−07 | A6 | Etnppl |
| Akap13 | 7.44E−12 | 0.44359 | 0.406 | 0.282 | 1.85E−07 | A6 | Akap13 |
| Wdr26 | 1.07E−11 | 0.37235 | 0.549 | 0.472 | 2.66E−07 | A6 | Wdr26 |
| Huwe1 | 1.08E−11 | 0.33358 | 0.492 | 0.38 | 2.69E−07 | A6 | Huwe1 |
| Cald1 | 1.16E−11 | 0.29347 | 0.766 | 0.795 | 2.89E−07 | A6 | Cald1 |
| Ankrd11 | 1.30E−11 | 0.37149 | 0.389 | 0.263 | 3.24E−07 | A6 | Ankrd11 |
| Mbnl1 | 2.38E−11 | 0.34827 | 0.492 | 0.394 | 5.91E−07 | A6 | Mbnl1 |
| Mug2 | 2.45E−11 | 0.2773 | 0.776 | 0.77 | 6.09E−07 | A6 | Mug2 |
| Rn7sk | 2.61E−11 | 0.2776 | 0.339 | 0.212 | 6.48E−07 | A6 | Rn7sk |
| Anxa5 | 2.84E−11 | 0.35799 | 0.391 | 0.278 | 7.05E−07 | A6 | Anxa5 |
| Metap2 | 4.08E−11 | 0.32796 | 0.453 | 0.353 | 1.01E−06 | A6 | Metap2 |
| Shb | 4.85E−11 | 0.28309 | 0.382 | 0.264 | 1.20E−06 | A6 | Shb |
| Gsr | 4.90E−11 | 0.39239 | 0.453 | 0.346 | 1.22E−06 | A6 | Gsr |
| Gsk3b | 5.45E−11 | 0.35405 | 0.384 | 0.272 | 1.35E−06 | A6 | Gsk3b |
| Kdelr2 | 5.86E−11 | 0.36902 | 0.484 | 0.413 | 1.45E−06 | A6 | Kdelr2 |
| Tjp1 | 1.36E−10 | 0.2529 | 0.251 | 0.142 | 3.37E−06 | A6 | Tjp1 |
| Sf1 | 2.46E−10 | 0.27771 | 0.351 | 0.233 | 6.11E−06 | A6 | Sf1 |
| Dhx9 | 3.77E−10 | 0.30561 | 0.427 | 0.319 | 9.37E−06 | A6 | Dhx9 |
| Smap2 | 4.08E−10 | 0.31706 | 0.363 | 0.246 | 1.01E−05 | A6 | Smap2 |
| Chmp4b | 4.61E−10 | 0.29391 | 0.442 | 0.344 | 1.14E−05 | A6 | Chmp4b |
| Cxcl1 | 5.14E−10 | 0.3092 | 0.444 | 0.334 | 1.28E−05 | A6 | Cxcl1 |
| Snx10 | 5.28E−10 | 0.26407 | 0.341 | 0.232 | 1.31E−05 | A6 | Snx10 |
| Pacsin2 | 5.77E−10 | 0.34257 | 0.37 | 0.264 | 1.43E−05 | A6 | Pacsin2 |
| Cast | 7.34E−10 | 0.34192 | 0.399 | 0.293 | 1.82E−05 | A6 | Cast |
| Akap11 | 7.52E−10 | 0.29723 | 0.351 | 0.24 | 1.87E−05 | A6 | Akap11 |
| Srsf11 | 7.58E−10 | 0.35423 | 0.418 | 0.313 | 1.88E−05 | A6 | Srsf11 |
| Reep3 | 9.04E−10 | 0.33507 | 0.535 | 0.502 | 2.24E−05 | A6 | Reep3 |
| mmu-mir-6236 | 1.28E−09 | 0.42047 | 0.831 | 0.853 | 3.19E−05 | A6 | mmu-mir-6236 |
| Atf4 | 2.02E−09 | 0.28671 | 0.58 | 0.505 | 5.02E−05 | A6 | Atf4 |
| Clic4 | 2.32E−09 | 0.2811 | 0.368 | 0.265 | 5.77E−05 | A6 | Clic4 |
| Ubr4 | 2.43E−09 | 0.29161 | 0.411 | 0.302 | 6.03E−05 | A6 | Ubr4 |
| Por | 2.58E−09 | 0.26872 | 0.704 | 0.721 | 6.42E−05 | A6 | Por |
| Bptf | 2.63E−09 | 0.28182 | 0.332 | 0.222 | 6.53E−05 | A6 | Bptf |
| Zbtb20 | 2.91E−09 | 0.37805 | 0.566 | 0.519 | 7.23E−05 | A6 | Zbtb20 |
| Dync1h1 | 3.26E−09 | 0.34196 | 0.379 | 0.275 | 8.10E−05 | A6 | Dync1h1 |
| Rlim | 3.27E−09 | 0.27576 | 0.305 | 0.199 | 8.12E−05 | A6 | Rlim |
| Birc6 | 4.85E−09 | 0.2922 | 0.446 | 0.34 | 0.0001204 | A6 | Birc6 |
| Atp1b1 | 5.41E−09 | 0.31397 | 0.391 | 0.294 | 0.0001342 | A6 | Atp1b1 |
| Slc25a47 | 6.75E−09 | 0.28843 | 0.869 | 0.931 | 0.0001677 | A6 | Slc25a47 |
| Slc25a51 | 7.23E−09 | 0.25329 | 0.308 | 0.203 | 0.0001795 | A6 | Slc25a51 |
| Jhdm1d | 7.29E−09 | 0.25415 | 0.279 | 0.177 | 0.0001811 | A6 | Jhdm1d |
| Steap4 | 9.00E−09 | 0.3653 | 0.37 | 0.275 | 0.0002235 | A6 | Steap4 |
| Mapkapk2 | 9.71E−09 | 0.3053 | 0.396 | 0.294 | 0.0002411 | A6 | Mapkapk2 |
| Gtf2a2 | 1.09E−08 | 0.2654 | 0.379 | 0.28 | 0.0002716 | A6 | Gtf2a2 |
| Slc4a4 | 1.12E−08 | 0.29527 | 0.513 | 0.439 | 0.0002785 | A6 | Slc4a4 |
| Tbl1xr1 | 1.79E−08 | 0.25532 | 0.341 | 0.246 | 0.0004436 | A6 | Tbl1xr1 |
| Sbno1 | 1.89E−08 | 0.29621 | 0.394 | 0.301 | 0.0004698 | A6 | Sbno1 |
| Nrp1 | 1.98E−08 | 0.32032 | 0.391 | 0.295 | 0.0004928 | A6 | Nrp1 |
| Tpr | 2.01E−08 | 0.35244 | 0.501 | 0.429 | 0.0004993 | A6 | Tpr |
| Kif21a | 2.01E−08 | 0.33336 | 0.461 | 0.377 | 0.0005003 | A6 | Kif21a |
| Mdm2 | 2.06E−08 | 0.27581 | 0.305 | 0.204 | 0.0005108 | A6 | Mdm2 |
| Ddx6 | 2.13E−08 | 0.34733 | 0.527 | 0.46 | 0.0005298 | A6 | Ddx6 |
| Rbm25 | 2.41E−08 | 0.36502 | 0.461 | 0.369 | 0.0005983 | A6 | Rbm25 |
| Uhrf1bp1l | 2.44E−08 | 0.31928 | 0.399 | 0.306 | 0.0006069 | A6 | Uhrf1bp1l |
| Cep85 | 3.36E−08 | 0.27083 | 0.313 | 0.215 | 0.0008335 | A6 | Cep85 |
| Hmgcr | 3.52E−08 | 0.3449 | 0.425 | 0.343 | 0.0008739 | A6 | Hmgcr |
| Saa2 | 4.22E−08 | 0.38921 | 0.315 | 0.221 | 0.0010472 | A6 | Saa2 |
| Tnks2 | 5.05E−08 | 0.26738 | 0.358 | 0.265 | 0.0012544 | A6 | Tnks2 |
| Net1 | 5.81E−08 | 0.32011 | 0.408 | 0.324 | 0.0014419 | A6 | Net1 |
| Rangap1 | 6.81E−08 | 0.27147 | 0.387 | 0.298 | 0.0016908 | A6 | Rangap1 |
| Atp13a3 | 8.04E−08 | 0.29804 | 0.532 | 0.494 | 0.0019974 | A6 | Atp13a3 |
| Tmed5 | 9.87E−08 | 0.26284 | 0.594 | 0.587 | 0.0024518 | A6 | Tmed5 |
| Atp2b1 | 1.14E−07 | 0.28938 | 0.334 | 0.239 | 0.0028301 | A6 | Atp2b1 |
| Ell2 | 1.24E−07 | 0.3073 | 0.535 | 0.508 | 0.0030817 | A6 | Ell2 |
| Eif4a1 | 1.50E−07 | 0.26079 | 0.418 | 0.331 | 0.0037153 | A6 | Eif4a1 |
| Macf1 | 1.58E−07 | 0.34781 | 0.377 | 0.287 | 0.0039148 | A6 | Macf1 |
| Srrm2 | 1.83E−07 | 0.28659 | 0.456 | 0.372 | 0.0045319 | A6 | Srrm2 |
| Brd2 | 1.86E−07 | 0.45982 | 0.542 | 0.493 | 0.0046088 | A6 | Brd2 |
| Ipo7 | 1.95E−07 | 0.26719 | 0.334 | 0.241 | 0.0048465 | A6 | Ipo7 |
| Cap1 | 2.19E−07 | 0.28532 | 0.356 | 0.261 | 0.0054372 | A6 | Cap1 |
| Lrrc59 | 2.71E−07 | 0.30238 | 0.422 | 0.354 | 0.00672 | A6 | Lrrc59 |
| Scaf11 | 3.09E−07 | 0.28756 | 0.449 | 0.382 | 0.0076803 | A6 | Scaf11 |
| Phf20l1 | 3.47E−07 | 0.26568 | 0.372 | 0.282 | 0.0086272 | A6 | Phf20l1 |
| Hipk3 | 3.52E−07 | 0.26049 | 0.396 | 0.309 | 0.0087524 | A6 | Hipk3 |
| Prpf19 | 3.65E−07 | 0.25658 | 0.432 | 0.356 | 0.0090721 | A6 | Prpf19 |
| Actr3 | 4.09E−07 | 0.26383 | 0.36 | 0.273 | 0.0101555 | A6 | Actr3 |
| Gfra1 | 5.26E−07 | 0.27582 | 0.599 | 0.583 | 0.0130598 | A6 | Gfra1 |
| Dnajc5 | 5.58E−07 | 0.26355 | 0.372 | 0.294 | 0.0138673 | A6 | Dnajc5 |
| Gm5424 | 7.53E−07 | 0.2647 | 0.411 | 0.325 | 0.0186934 | A6 | Gm5424 |
| Ppip5k2 | 7.82E−07 | 0.30024 | 0.344 | 0.254 | 0.0194128 | A6 | Ppip5k2 |
| Yap1 | 8.00E−07 | 0.26153 | 0.375 | 0.288 | 0.019873 | A6 | Yap1 |
| mt-Co1 | 1.20E−06 | 0.35102 | 0.998 | 0.988 | 0.0297443 | A6 | mt-Co1 |
| Mgea5 | 1.25E−06 | 0.29042 | 0.413 | 0.333 | 0.0310796 | A6 | Mgea5 |
| Kpna1 | 1.55E−06 | 0.25545 | 0.363 | 0.286 | 0.0384703 | A6 | Kpna1 |
| Atf6 | 1.79E−06 | 0.29222 | 0.432 | 0.365 | 0.0444322 | A6 | Atf6 |
| Ivns1abp | 1.94E−06 | 0.27551 | 0.446 | 0.398 | 0.0482394 | A6 | Ivns1abp |
| Dnaja2 | 2.46E−06 | 0.29931 | 0.516 | 0.482 | 0.0612092 | A6 | Dnaja2 |
| Ctsc | 2.59E−06 | 0.25762 | 0.403 | 0.321 | 0.064323 | A6 | Ctsc |
| Kdm5a | 2.84E−06 | 0.31018 | 0.308 | 0.221 | 0.0705376 | A6 | Kdm5a |
| Abce1 | 2.87E−06 | 0.25023 | 0.437 | 0.365 | 0.0712986 | A6 | Abce1 |
| Rsl1d1 | 3.31E−06 | 0.27488 | 0.47 | 0.423 | 0.0820794 | A6 | Rsl1d1 |
| Eprs | 4.18E−06 | 0.29766 | 0.566 | 0.558 | 0.1038218 | A6 | Eprs |
| Hnrnpu | 5.29E−06 | 0.25058 | 0.625 | 0.651 | 0.1313752 | A6 | Hnrnpu |
| H6pd | 5.50E−06 | 0.25615 | 0.623 | 0.652 | 0.1366428 | A6 | H6pd |
| mt-Nd5 | 6.52E−06 | 0.34113 | 0.983 | 0.969 | 0.1619437 | A6 | mt-Nd5 |
| Rnf169 | 7.00E−06 | 0.29632 | 0.315 | 0.235 | 0.1737208 | A6 | Rnf169 |
| Rap1b | 7.33E−06 | 0.28412 | 0.406 | 0.335 | 0.1819444 | A6 | Rap1b |
| Chuk | 8.66E−06 | 0.29548 | 0.523 | 0.495 | 0.2149235 | A6 | Chuk |
| Sf3b1 | 9.81E−06 | 0.25061 | 0.508 | 0.475 | 0.2435822 | A6 | Sf3b1 |
| Serinc3 | 1.22E−05 | 0.28119 | 0.525 | 0.498 | 0.3039179 | A6 | Serinc3 |
| Pi4k2a | 1.25E−05 | 0.32702 | 0.425 | 0.381 | 0.3103073 | A6 | Pi4k2a |
| Alcam | 1.35E−05 | 0.26477 | 0.396 | 0.329 | 0.334205 | A6 | Alcam |
| Gadd45a | 1.39E−05 | 0.28209 | 0.251 | 0.178 | 0.3447616 | A6 | Gadd45a |
| Diap1 | 1.79E−05 | 0.28157 | 0.382 | 0.306 | 0.4448124 | A6 | Diap1 |
| Ddx3y | 3.35E−05 | 0.27782 | 0.47 | 0.43 | 0.8318918 | A6 | Ddx3y |
| Eif4b | 3.52E−05 | 0.27888 | 0.542 | 0.521 | 0.8734269 | A6 | Eif4b |
| Nploc4 | 5.58E−05 | 0.25153 | 0.351 | 0.28 | 1 | A6 | Nploc4 |
| Nucks1 | 7.26E−05 | 0.26038 | 0.532 | 0.517 | 1 | A6 | Nucks1 |
| Tab2 | 7.82E−05 | 0.29307 | 0.425 | 0.374 | 1 | A6 | Tab2 |
| Slc35d1 | 8.85E−05 | 0.25379 | 0.489 | 0.496 | 1 | A6 | Slc35d1 |
| Abcc2 | 8.98E−05 | 0.26003 | 0.632 | 0.654 | 1 | A6 | Abcc2 |
| Atp1a1 | 9.45E−05 | 0.29131 | 0.53 | 0.538 | 1 | A6 | Atp1a1 |
| Slc40a1 | 0.00029608 | 0.34995 | 0.456 | 0.417 | 1 | A6 | Slc40a1 |
| Zfp106 | 0.00031964 | 0.27067 | 0.391 | 0.333 | 1 | A6 | Zfp106 |
| Ier2 | 0.00036702 | 0.25075 | 0.551 | 0.517 | 1 | A6 | Ier2 |
| Trip11 | 0.00058924 | 0.28702 | 0.341 | 0.284 | 1 | A6 | Trip11 |
| Srsf5 | 0.00064161 | 0.25349 | 0.387 | 0.34 | 1 | A6 | Srsf5 |
| Spag9 | 0.00172919 | 0.25323 | 0.415 | 0.373 | 1 | A6 | Spag9 |
| Tln1 | 0.00250885 | 0.2937 | 0.31 | 0.259 | 1 | A6 | Tln1 |
| Riok3 | 0.0030185 | 0.25892 | 0.43 | 0.406 | 1 | A6 | Riok3 |
| mt-Nd6 | 0.00348624 | 0.29062 | 0.527 | 0.502 | 1 | A6 | mt-Nd6 |
| Gm269241 | 0 | 1.25316 | 1 | 1 | 0 | A24 | Gm26924 |
| n-R5-8s11 | 0 | 1.15081 | 0.522 | 0.075 | 0 | A24 | n-R5-8s1 |
| Grip2 | 0 | 0.87737 | 0.475 | 0.003 | 0 | A24 | Grip2 |
| Fga1 | 0 | 0.76204 | 0.998 | 0.996 | 0 | A24 | Fga |
| Fgb1 | 0 | 0.68103 | 1 | 0.999 | 0 | A24 | Fgb |
| Fgg1 | 0 | 0.6334 | 0.999 | 0.993 | 0 | A24 | Fgg |
| Lars21 | 1.08E−286 | 1.49313 | 0.998 | 0.995 | 2.69E−282 | A24 | Lars2 |
| Sephs21 | 1.58E−286 | 0.85357 | 0.949 | 0.785 | 3.91E−282 | A24 | Sephs2 |
| Fgl1 | 5.87E−275 | 0.87406 | 0.938 | 0.799 | 1.46E−270 | A24 | Fgl1 |
| Gm24601 | 4.02E−274 | 0.93244 | 0.404 | 0.047 | 9.99E−270 | A24 | Gm24601 |
| Gm155641 | 3.89E−272 | 1.55543 | 0.982 | 0.965 | 9.67E−268 | A24 | Gm15564 |
| Calr1 | 5.92E−193 | 0.61158 | 0.952 | 0.882 | 1.47E−188 | A24 | Calr |
| Pdia31 | 3.00E−171 | 0.64241 | 0.895 | 0.805 | 7.45E−167 | A24 | Pdia3 |
| Hyou1 | 1.92E−169 | 0.84359 | 0.717 | 0.423 | 4.77E−165 | A24 | Hyou1 |
| Saa21 | 1.56E−157 | 1.0091 | 0.52 | 0.172 | 3.88E−153 | A24 | Saa2 |
| Hsp90b11 | 2.63E−155 | 0.54513 | 0.954 | 0.948 | 6.53E−151 | A24 | Hsp90b1 |
| Sepp1 | 5.04E−153 | 0.32603 | 1 | 1 | 1.25E−148 | A24 | Sepp1 |
| Hspa5 | 1.66E−152 | 0.48995 | 0.974 | 0.954 | 4.12E−148 | A24 | Hspa5 |
| Orm1 | 6.97E−135 | 0.46612 | 0.963 | 0.942 | 1.73E−130 | A24 | Orm1 |
| Itih4 | 2.79E−129 | 0.4689 | 0.974 | 0.956 | 6.93E−125 | A24 | Itih4 |
| Egfr | 2.69E−128 | 0.62342 | 0.882 | 0.802 | 6.67E−124 | A24 | Egfr |
| Glud1 | 1.70E−115 | 0.42115 | 0.958 | 0.921 | 4.22E−111 | A24 | Glud1 |
| HP | 2.71E−113 | 0.36435 | 0.998 | 0.999 | 6.73E−109 | A24 | HP |
| Mup71 | 1.68E−111 | 0.26813 | 0.985 | 0.965 | 4.16E−107 | A24 | Mup7 |
| Serpina101 | 2.00E−108 | 0.57073 | 0.813 | 0.667 | 4.97E−104 | A24 | Serpina10 |
| Rnase41 | 2.35E−108 | 0.55401 | 0.963 | 0.898 | 5.83E−104 | A24 | Rnase4 |
| Scd11 | 2.78E−104 | 0.55857 | 0.98 | 0.925 | 6.90E−100 | A24 | Scd1 |
| Eef1a1 | 2.17E−101 | 0.45614 | 0.919 | 0.858 | 5.38E−97 | A24 | Eef1a1 |
| Saa1 | 2.48E−100 | 0.78117 | 0.792 | 0.549 | 6.16E−96 | A24 | Saa1 |
| C8b1 | 1.53E−99 | 0.44014 | 0.898 | 0.871 | 3.79E−95 | A24 | C8b |
| Hdlbp | 2.71E−98 | 0.52785 | 0.853 | 0.753 | 6.72E−94 | A24 | Hdlbp |
| Cyp2c701 | 7.78E−90 | 0.47953 | 0.89 | 0.823 | 1.93E−85 | A24 | Cyp2c70 |
| P4hb1 | 2.74E−89 | 0.33254 | 0.973 | 0.945 | 6.82E−85 | A24 | P4hb |
| Apcs | 5.47E−89 | 0.60591 | 0.738 | 0.556 | 1.36E−84 | A24 | Apcs |
| Creld2 | 5.15E−88 | 0.59406 | 0.583 | 0.352 | 1.28E−83 | A24 | Creld2 |
| C91 | 2.56E−84 | 0.37576 | 0.952 | 0.926 | 6.35E−80 | A24 | C9 |
| Orm2 | 5.62E−84 | 0.30465 | 0.285 | 0.081 | 1.40E−79 | A24 | Orm2 |
| Ssr3 | 1.77E−83 | 0.48612 | 0.765 | 0.642 | 4.40E−79 | A24 | Ssr3 |
| Pzp | 4.35E−83 | 0.35431 | 0.984 | 0.988 | 1.08E−78 | A24 | Pzp |
| C31 | 2.09E−82 | 0.32381 | 0.996 | 0.993 | 5.18E−78 | A24 | C3 |
| Arrdc31 | 1.19E−81 | 0.49892 | 0.52 | 0.272 | 2.97E−77 | A24 | Arrdc3 |
| Saa41 | 1.26E−81 | 0.47628 | 0.823 | 0.734 | 3.13E−77 | A24 | Saa4 |
| Fn11 | 3.93E−80 | 0.55363 | 0.905 | 0.858 | 9.76E−76 | A24 | Fn1 |
| Cfh | 3.78E−79 | 0.45472 | 0.921 | 0.904 | 9.38E−75 | A24 | Cfh |
| Cat | 3.96E−78 | 0.28564 | 0.978 | 0.977 | 9.84E−74 | A24 | Cat |
| Actb1 | 4.53E−78 | 0.32283 | 0.919 | 0.895 | 1.12E−73 | A24 | Actb |
| Pdia41 | 2.83E−76 | 0.52087 | 0.733 | 0.616 | 7.02E−72 | A24 | Pdia4 |
| Sc5d1 | 4.88E−76 | 0.39881 | 0.879 | 0.797 | 1.21E−71 | A24 | Sc5d |
| Steap41 | 5.67E−76 | 0.46634 | 0.481 | 0.243 | 1.41E−71 | A24 | Steap4 |
| C8a | 1.03E−72 | 0.424 | 0.902 | 0.877 | 2.55E−68 | A24 | C8a |
| Pgrmc1 | 1.30E−70 | 0.30757 | 0.953 | 0.947 | 3.22E−66 | A24 | Pgrmc1 |
| Itih31 | 3.45E−70 | 0.39323 | 0.931 | 0.909 | 8.58E−66 | A24 | Itih3 |
| Nudt71 | 3.41E−69 | 0.34726 | 0.884 | 0.74 | 8.47E−65 | A24 | Nudt7 |
| Hc | 5.21E−67 | 0.38899 | 0.905 | 0.904 | 1.29E−62 | A24 | Hc |
| Tram1 | 1.87E−66 | 0.42942 | 0.7 | 0.545 | 4.63E−62 | A24 | Tram1 |
| Myh91 | 1.24E−65 | 0.49194 | 0.595 | 0.388 | 3.08E−61 | A24 | Myh9 |
| Mup121 | 1.65E−65 | 0.45328 | 0.799 | 0.759 | 4.10E−61 | A24 | Mup12 |
| Gm269171 | 8.37E−65 | 0.78706 | 0.391 | 0.183 | 2.08E−60 | A24 | Gm26917 |
| Dnajc3 | 2.12E−64 | 0.38899 | 0.85 | 0.808 | 5.26E−60 | A24 | Dnajc3 |
| Hao1 | 7.24E−61 | 0.39162 | 0.83 | 0.767 | 1.80E−56 | A24 | Hao1 |
| Pdia61 | 8.25E−60 | 0.42093 | 0.779 | 0.702 | 2.05E−55 | A24 | Pdia6 |
| Apob1 | 9.57E−60 | 0.35398 | 0.983 | 0.99 | 2.38E−55 | A24 | Apob |
| Manf | 2.64E−58 | 0.46525 | 0.632 | 0.478 | 6.55E−54 | A24 | Manf |
| Pdcd41 | 4.76E−58 | 0.46791 | 0.677 | 0.542 | 1.18E−53 | A24 | Pdcd4 |
| AI1823711 | 9.61E−58 | 0.42306 | 0.747 | 0.654 | 2.39E−53 | A24 | AI182371 |
| Comt1 | 5.49E−57 | 0.30202 | 0.928 | 0.92 | 1.36E−52 | A24 | Comt |
| Cpb21 | 2.17E−56 | 0.33155 | 0.879 | 0.827 | 5.40E−52 | A24 | Cpb2 |
| Serp1 | 2.18E−55 | 0.40205 | 0.809 | 0.725 | 5.42E−51 | A24 | Serp1 |
| Cml21 | 5.80E−55 | 0.34556 | 0.896 | 0.849 | 1.44E−50 | A24 | Cml2 |
| Elovl2 | 1.73E−53 | 0.38067 | 0.826 | 0.751 | 4.29E−49 | A24 | Elovl2 |
| mmu-mir-62361 | 6.37E−53 | 0.84589 | 0.896 | 0.843 | 1.58E−48 | A24 | mmu-mir-6236 |
| Iigp11 | 1.35E−52 | 0.33355 | 0.832 | 0.798 | 3.36E−48 | A24 | Iigp1 |
| Calm1 | 1.03E−50 | 0.37016 | 0.794 | 0.759 | 2.56E−46 | A24 | Calm1 |
| Cps1 | 2.48E−50 | 0.27751 | 0.982 | 0.977 | 6.16E−46 | A24 | Cps1 |
| Eif4g21 | 6.14E−50 | 0.35147 | 0.8 | 0.739 | 1.52E−45 | A24 | Eif4g2 |
| Acox1 | 1.63E−48 | 0.27431 | 0.966 | 0.968 | 4.05E−44 | A24 | Acox1 |
| Fndc3b1 | 3.80E−48 | 0.32024 | 0.291 | 0.125 | 9.44E−44 | A24 | Fndc3b |
| Slc41a2 | 1.01E−47 | 0.28796 | 0.302 | 0.132 | 2.51E−43 | A24 | Slc41a2 |
| Nr1d2 | 5.02E−47 | 0.38547 | 0.442 | 0.263 | 1.25E−42 | A24 | Nr1d2 |
| Insig1 | 1.30E−46 | 0.41247 | 0.626 | 0.487 | 3.23E−42 | A24 | Insig1 |
| Aox31 | 1.16E−45 | 0.34889 | 0.916 | 0.896 | 2.88E−41 | A24 | Aox3 |
| Spcs3 | 6.70E−45 | 0.39071 | 0.483 | 0.325 | 1.66E−40 | A24 | Spcs3 |
| Atxn1 | 2.39E−44 | 0.32504 | 0.311 | 0.148 | 5.93E−40 | A24 | Atxn1 |
| Cmah | 3.14E−44 | 0.42429 | 0.607 | 0.472 | 7.80E−40 | A24 | Cmah |
| Pnpla7 | 4.00E−44 | 0.39659 | 0.678 | 0.561 | 9.93E−40 | A24 | Pnpla7 |
| Dnajc12 | 1.33E−42 | 0.32405 | 0.275 | 0.125 | 3.31E−38 | A24 | Dnajc12 |
| Atp8b1 | 3.45E−42 | 0.36101 | 0.391 | 0.221 | 8.56E−38 | A24 | Atp8b1 |
| Hspa13 | 3.59E−42 | 0.34197 | 0.404 | 0.237 | 8.92E−38 | A24 | Hspa13 |
| AA4744081 | 1.41E−41 | 0.41482 | 0.278 | 0.126 | 3.51E−37 | A24 | AA474408 |
| Canx | 1.09E−40 | 0.27706 | 0.872 | 0.862 | 2.70E−36 | A24 | Canx |
| Tmed7 | 1.35E−40 | 0.3715 | 0.563 | 0.417 | 3.35E−36 | A24 | Tmed7 |
| Il13ra1 | 1.42E−40 | 0.28891 | 0.33 | 0.168 | 3.53E−36 | A24 | Il13ra1 |
| Sdf2l1 | 2.54E−40 | 0.30777 | 0.412 | 0.24 | 6.31E−36 | A24 | Sdf2l1 |
| Acly | 5.13E−40 | 0.41013 | 0.597 | 0.448 | 1.27E−35 | A24 | Acly |
| Cxcl11 | 8.60E−40 | 0.45301 | 0.482 | 0.315 | 2.14E−35 | A24 | Cxcl1 |
| Prg4 | 3.70E−39 | 0.4007 | 0.421 | 0.254 | 9.19E−35 | A24 | Prg4 |
| Adh41 | 9.82E−39 | 0.39197 | 0.667 | 0.538 | 2.44E−34 | A24 | Adh4 |
| Lrg11 | 4.41E−38 | 0.30956 | 0.849 | 0.786 | 1.09E−33 | A24 | Lrg1 |
| Hsd17b21 | 1.09E−37 | 0.32314 | 0.758 | 0.682 | 2.72E−33 | A24 | Hsd17b2 |
| Hsd3b51 | 1.25E−36 | 0.30091 | 0.638 | 0.451 | 3.10E−32 | A24 | Hsd3b5 |
| Hs3st3b1 | 3.00E−36 | 0.3219 | 0.442 | 0.286 | 7.44E−32 | A24 | Hs3st3b1 |
| Rpn1 | 3.41E−36 | 0.39915 | 0.605 | 0.494 | 8.47E−32 | A24 | Rpn1 |
| Tmem30a | 9.46E−36 | 0.30562 | 0.766 | 0.712 | 2.35E−31 | A24 | Tmem30a |
| Rrbp1 | 2.26E−35 | 0.29675 | 0.823 | 0.794 | 5.62E−31 | A24 | Rrbp1 |
| Cyb5r3 | 9.28E−35 | 0.30966 | 0.8 | 0.757 | 2.30E−30 | A24 | Cyb5r3 |
| Hnf4a1 | 9.32E−34 | 0.25495 | 0.85 | 0.799 | 2.31E−29 | A24 | Hnf4a |
| Fos | 1.67E−33 | 0.36319 | 0.654 | 0.54 | 4.16E−29 | A24 | Fos |
| Abca11 | 3.07E−33 | 0.38309 | 0.556 | 0.428 | 7.62E−29 | A24 | Abca1 |
| Cltc1 | 1.19E−32 | 0.33967 | 0.61 | 0.485 | 2.96E−28 | A24 | Cltc |
| Qsox1 | 6.53E−32 | 0.28175 | 0.784 | 0.741 | 1.62E−27 | A24 | Qsox1 |
| Lrp1 | 2.33E−31 | 0.48374 | 0.694 | 0.626 | 5.78E−27 | A24 | Lrp1 |
| Mlec | 2.33E−31 | 0.34053 | 0.496 | 0.361 | 5.78E−27 | A24 | Mlec |
| Ssr1 | 3.21E−31 | 0.31287 | 0.523 | 0.389 | 7.96E−27 | A24 | Ssr1 |
| Nrp11 | 1.23E−30 | 0.32998 | 0.426 | 0.278 | 3.05E−26 | A24 | Nrp1 |
| Ostc | 3.89E−30 | 0.30819 | 0.514 | 0.373 | 9.65E−26 | A24 | Ostc |
| Kmo1 | 9.66E−30 | 0.2687 | 0.778 | 0.756 | 2.40E−25 | A24 | Kmo |
| Ctnnb1 | 7.11E−29 | 0.32898 | 0.566 | 0.457 | 1.77E−24 | A24 | Ctnnb1 |
| Uggt1 | 9.70E−29 | 0.28054 | 0.385 | 0.245 | 2.41E−24 | A24 | Uggt1 |
| Gjb21 | 1.47E−28 | 0.2998 | 0.709 | 0.635 | 3.66E−24 | A24 | Gjb2 |
| Uso1 | 2.20E−28 | 0.32023 | 0.597 | 0.51 | 5.47E−24 | A24 | Uso1 |
| Slc3a1 | 2.88E−28 | 0.30744 | 0.378 | 0.24 | 7.15E−24 | A24 | Slc3a1 |
| Efna1 | 5.06E−28 | 0.31613 | 0.482 | 0.361 | 1.26E−23 | A24 | Efna1 |
| Usp9x | 1.17E−27 | 0.33869 | 0.502 | 0.378 | 2.91E−23 | A24 | Usp9x |
| Sec61b1 | 2.12E−27 | 0.30077 | 0.549 | 0.435 | 5.27E−23 | A24 | Sec61b |
| Calu | 4.28E−27 | 0.30047 | 0.407 | 0.276 | 1.06E−22 | A24 | Calu |
| Ak3 | 4.61E−27 | 0.25045 | 0.772 | 0.74 | 1.14E−22 | A24 | Ak3 |
| Nedd4 | 1.15E−26 | 0.33055 | 0.647 | 0.583 | 2.86E−22 | A24 | Nedd4 |
| Usp4 | 5.04E−26 | 0.2959 | 0.476 | 0.347 | 1.25E−21 | A24 | Usp4 |
| Gm54241 | 8.58E−26 | 0.26199 | 0.445 | 0.309 | 2.13E−21 | A24 | Gm5424 |
| Dhcr24 | 1.11E−25 | 0.28238 | 0.857 | 0.827 | 2.77E−21 | A24 | Dhcr24 |
| Rab1 | 1.32E−25 | 0.31448 | 0.588 | 0.498 | 3.29E−21 | A24 | Rab1 |
| Eif4g11 | 3.05E−25 | 0.27629 | 0.745 | 0.715 | 7.57E−21 | A24 | Eif4g1 |
| Acsl4 | 5.14E−25 | 0.29904 | 0.478 | 0.365 | 1.28E−20 | A24 | Acsl4 |
| Dnajb11 | 1.54E−24 | 0.32878 | 0.557 | 0.467 | 3.83E−20 | A24 | Dnajb11 |
| Srebf1 | 1.85E−24 | 0.31485 | 0.439 | 0.319 | 4.59E−20 | A24 | Srebf1 |
| Arcn1 | 1.92E−24 | 0.3044 | 0.53 | 0.421 | 4.76E−20 | A24 | Arcn1 |
| Copa | 6.77E−24 | 0.33341 | 0.525 | 0.417 | 1.68E−19 | A24 | Copa |
| Mug21 | 1.27E−23 | 0.31662 | 0.784 | 0.767 | 3.15E−19 | A24 | Mug2 |
| Il6st | 1.30E−23 | 0.3011 | 0.448 | 0.329 | 3.23E−19 | A24 | Il6st |
| Etnk1 | 2.89E−23 | 0.31944 | 0.49 | 0.384 | 7.17E−19 | A24 | Etnk1 |
| Prkd31 | 3.51E−23 | 0.30413 | 0.631 | 0.557 | 8.72E−19 | A24 | Prkd3 |
| Rdx1 | 4.33E−23 | 0.27601 | 0.729 | 0.695 | 1.07E−18 | A24 | Rdx |
| Ibtk | 5.93E−23 | 0.2959 | 0.43 | 0.312 | 1.47E−18 | A24 | Ibtk |
| Lgr4 | 1.20E−22 | 0.25721 | 0.399 | 0.279 | 2.99E−18 | A24 | Lgr4 |
| Srpr | 5.67E−22 | 0.29141 | 0.532 | 0.442 | 1.41E−17 | A24 | Srpr |
| Lman11 | 6.76E−22 | 0.25181 | 0.75 | 0.697 | 1.68E−17 | A24 | Lman1 |
| Edem1 | 1.41E−20 | 0.26754 | 0.566 | 0.477 | 3.50E−16 | A24 | Edem1 |
| Pten | 1.56E−20 | 0.26629 | 0.612 | 0.536 | 3.87E−16 | A24 | Pten |
| Lrrc591 | 5.25E−20 | 0.26877 | 0.449 | 0.341 | 1.30E−15 | A24 | Lrrc59 |
| Srp72 | 6.96E−20 | 0.27999 | 0.597 | 0.524 | 1.73E−15 | A24 | Srp72 |
| Nars1 | 1.10E−19 | 0.31271 | 0.593 | 0.534 | 2.74E−15 | A24 | Nars |
| Ptprf1 | 1.19E−19 | 0.28883 | 0.588 | 0.514 | 2.95E−15 | A24 | Ptprf |
| Trip12 | 1.43E−19 | 0.28318 | 0.504 | 0.407 | 3.54E−15 | A24 | Trip12 |
| Mtus1 | 1.55E−19 | 0.31246 | 0.563 | 0.485 | 3.85E−15 | A24 | Mtus1 |
| Stard4 | 2.95E−19 | 0.29219 | 0.559 | 0.471 | 7.32E−15 | A24 | Stard4 |
| Proz | 3.55E−19 | 0.27997 | 0.622 | 0.56 | 8.83E−15 | A24 | Proz |
| Txndc5 | 6.96E−19 | 0.27583 | 0.48 | 0.378 | 1.73E−14 | A24 | Txndc5 |
| Echdc1 | 8.90E−19 | 0.28178 | 0.503 | 0.423 | 2.21E−14 | A24 | Echdc1 |
| Gm242451 | 1.07E−18 | 0.41399 | 0.369 | 0.262 | 2.65E−14 | A24 | Gm24245 |
| Sec31a | 1.72E−18 | 0.2596 | 0.462 | 0.361 | 4.27E−14 | A24 | Sec31a |
| Sh3bgrl | 2.81E−18 | 0.26679 | 0.512 | 0.42 | 6.99E−14 | A24 | Sh3bgrl |
| Mtss1 | 4.50E−18 | 0.25221 | 0.581 | 0.508 | 1.12E−13 | A24 | Mtss1 |
| Sel1l | 7.10E−18 | 0.28335 | 0.544 | 0.46 | 1.76E−13 | A24 | Sel1l |
| Ddost | 8.86E−18 | 0.2909 | 0.493 | 0.411 | 2.20E−13 | A24 | Ddost |
| Slc38a10 | 2.68E−17 | 0.25566 | 0.449 | 0.354 | 6.66E−13 | A24 | Slc38a10 |
| Nab1 | 5.44E−17 | 0.26005 | 0.491 | 0.398 | 1.35E−12 | A24 | Nab1 |
| Nr5a2 | 9.20E−17 | 0.2599 | 0.403 | 0.305 | 2.28E−12 | A24 | Nr5a2 |
| Met | 1.57E−16 | 0.25118 | 0.434 | 0.344 | 3.91E−12 | A24 | Met |
| Stt3b | 1.78E−15 | 0.26482 | 0.485 | 0.404 | 4.42E−11 | A24 | Stt3b |
| Atp11c | 2.49E−15 | 0.25676 | 0.649 | 0.621 | 6.17E−11 | A24 | Atp11c |
| Lasp1 | 2.85E−15 | 0.2608 | 0.361 | 0.268 | 7.09E−11 | A24 | Lasp1 |
| Grlf1 | 5.07E−15 | 0.25556 | 0.413 | 0.321 | 1.26E−10 | A24 | Grlf1 |
| Fam120a | 2.45E−14 | 0.25828 | 0.51 | 0.444 | 6.09E−10 | A24 | Fam120a |
| Eprs1 | 1.69E−12 | 0.25129 | 0.587 | 0.553 | 4.20E−08 | A24 | Eprs |
| Lpgat11 | 2.69E−12 | 0.2525 | 0.635 | 0.587 | 6.67E−08 | A24 | Lpgat1 |
| Zbtb201 | 5.86E−12 | 0.31348 | 0.566 | 0.513 | 1.46E−07 | A24 | Zbtb20 |
| Fasn | 5.64E−09 | 0.2699 | 0.449 | 0.394 | 0.0001401 | A24 | Fasn |
| Cyp4a142 | 0 | 1.28082 | 0.991 | 0.5 | 0 | A48 | Cyp4a14 |
| Mat1a2 | 0 | 0.77534 | 1 | 0.97 | 0 | A48 | Mat1a |
| Hmgcs22 | 0 | 0.76866 | 1 | 0.977 | 0 | A48 | Hmgcs2 |
| Apoe2 | 1.02E−282 | 0.50485 | 1 | 1 | 2.53E−278 | A48 | Apoe |
| Ces1c2 | 1.85E−278 | 0.54878 | 0.999 | 0.984 | 4.59E−274 | A48 | Ces1c |
| Cyp4a101 | 2.26E−270 | 0.93258 | 0.991 | 0.643 | 5.62E−266 | A48 | Cyp4a10 |
| Chchd102 | 1.01E−265 | 0.75168 | 0.995 | 0.925 | 2.50E−261 | A48 | Chchd10 |
| Apoc21 | 4.27E−263 | 0.86385 | 0.98 | 0.8 | 1.06E−258 | A48 | Apoc2 |
| Mfsd2a2 | 8.03E−247 | 0.66138 | 0.731 | 0.203 | 1.99E−242 | A48 | Mfsd2a |
| Ttc362 | 5.87E−246 | 0.56379 | 0.996 | 0.93 | 1.46E−241 | A48 | Ttc36 |
| Angptl32 | 1.51E−240 | 0.61108 | 0.995 | 0.946 | 3.74E−236 | A48 | Angptl3 |
| Apoh2 | 2.05E−235 | 0.46279 | 0.998 | 0.979 | 5.10E−231 | A48 | Apoh |
| Dbp | 5.52E−234 | 0.28956 | 0.52 | 0.091 | 1.37E−229 | A48 | Dbp |
| Ahsg1 | 5.89E−227 | 0.35916 | 1 | 0.999 | 1.46E−222 | A48 | Ahsg |
| Vtn1 | 2.03E−225 | 0.52281 | 1 | 0.98 | 5.04E−221 | A48 | Vtn |
| Rgs16 | 5.76E−225 | 0.45446 | 0.601 | 0.138 | 1.43E−220 | A48 | Rgs16 |
| Retsat2 | 2.28E−218 | 0.70957 | 0.988 | 0.827 | 5.66E−214 | A48 | Retsat |
| Abcd2 | 2.96E−203 | 0.2813 | 0.473 | 0.087 | 7.35E−199 | A48 | Abcd2 |
| Agt1 | 5.52E−203 | 0.57953 | 0.981 | 0.814 | 1.37E−198 | A48 | Agt |
| Herpud11 | 1.06E−202 | 0.60438 | 0.985 | 0.795 | 2.64E−198 | A48 | Herpud1 |
| Aldob | 1.64E−196 | 0.4375 | 1 | 0.996 | 4.06E−192 | A48 | Aldob |
| Serping11 | 3.48E−195 | 0.57771 | 0.987 | 0.863 | 8.63E−191 | A48 | Serping1 |
| Reep61 | 1.30E−193 | 0.62329 | 0.967 | 0.791 | 3.24E−189 | A48 | Reep6 |
| Apoa51 | 1.41E−191 | 0.44835 | 0.998 | 0.961 | 3.49E−187 | A48 | Apoa5 |
| Decr12 | 3.46E−191 | 0.56691 | 0.96 | 0.678 | 8.60E−187 | A48 | Decr1 |
| Slc25a472 | 1.27E−188 | 0.61748 | 0.992 | 0.917 | 3.16E−184 | A48 | Slc25a47 |
| F2 | 2.67E−188 | 0.39515 | 0.999 | 0.969 | 6.63E−184 | A48 | F2 |
| Rdh72 | 6.84E−187 | 0.46844 | 0.996 | 0.958 | 1.70E−182 | A48 | Rdh7 |
| Hrg1 | 3.57E−183 | 0.53157 | 0.987 | 0.834 | 8.86E−179 | A48 | Hrg |
| Rbp41 | 4.56E−183 | 0.39421 | 1 | 0.997 | 1.13E−178 | A48 | Rbp4 |
| Tef | 4.84E−177 | 0.33942 | 0.651 | 0.192 | 1.20E−172 | A48 | Tef |
| Slco1a4 | 2.11E−176 | 0.50909 | 0.77 | 0.291 | 5.23E−172 | A48 | Slco1a4 |
| Gc | 3.39E−175 | 0.29789 | 1 | 0.998 | 8.41E−171 | A48 | Gc |
| Gm6484 | 3.94E−173 | 0.41077 | 0.627 | 0.191 | 9.79E−169 | A48 | Gm6484 |
| Gm23935 | 1.27E−167 | 0.47384 | 0.997 | 0.93 | 3.15E−163 | A48 | Gm23935 |
| Ugt2b52 | 3.54E−167 | 0.43882 | 0.996 | 0.942 | 8.78E−163 | A48 | Ugt2b5 |
| Ttr2 | 2.12E−165 | 0.42249 | 1 | 0.999 | 5.26E−161 | A48 | Ttr |
| Rpl38 | 1.26E−162 | 0.3322 | 0.596 | 0.173 | 3.14E−158 | A48 | Rpl38 |
| St3gal52 | 9.27E−162 | 0.55493 | 0.914 | 0.537 | 2.30E−157 | A48 | St3gal5 |
| Lcat1 | 4.41E−155 | 0.50535 | 0.973 | 0.77 | 1.10E−150 | A48 | Lcat |
| Atp5h2 | 8.78E−153 | 0.59775 | 0.933 | 0.64 | 2.18E−148 | A48 | Atp5h |
| Apoc32 | 4.46E−151 | 0.40698 | 1 | 0.977 | 1.11E−146 | A48 | Apoc3 |
| Cp | 1.69E−147 | 0.42739 | 0.995 | 0.947 | 4.21E−143 | A48 | Cp |
| Plin51 | 4.30E−146 | 0.3985 | 0.788 | 0.332 | 1.07E−141 | A48 | Plin5 |
| Prodh21 | 2.43E−145 | 0.50547 | 0.918 | 0.598 | 6.02E−141 | A48 | Prodh2 |
| Oaf1 | 3.43E−145 | 0.50208 | 0.969 | 0.783 | 8.51E−141 | A48 | Oaf |
| Scand1 | 3.53E−145 | 0.27068 | 0.579 | 0.175 | 8.77E−141 | A48 | Scand1 |
| Khk2 | 6.78E−145 | 0.52025 | 0.982 | 0.855 | 1.68E−140 | A48 | Khk |
| Hmgcl2 | 1.35E−144 | 0.55008 | 0.956 | 0.723 | 3.36E−140 | A48 | Hmgcl |
| Lims2 | 6.42E−144 | 0.37612 | 0.679 | 0.253 | 1.59E−139 | A48 | Lims2 |
| Pnpla21 | 9.86E−144 | 0.3465 | 0.687 | 0.253 | 2.45E−139 | A48 | Pnpla2 |
| Sult1a11 | 4.81E−142 | 0.48266 | 0.963 | 0.718 | 1.20E−137 | A48 | Sult1a1 |
| Fth1 | 1.45E−141 | 0.37501 | 0.996 | 0.975 | 3.60E−137 | A48 | Fth1 |
| Lpin11 | 9.79E−141 | 0.51496 | 0.736 | 0.315 | 2.43E−136 | A48 | Lpin1 |
| Hdac111 | 4.34E−139 | 0.35573 | 0.713 | 0.277 | 1.08E−134 | A48 | Hdac11 |
| Syvn1 | 4.46E−139 | 0.30412 | 0.603 | 0.201 | 1.11E−134 | A48 | Syvn1 |
| Ttpa | 1.70E−138 | 0.44315 | 0.991 | 0.876 | 4.23E−134 | A48 | Ttpa |
| Nr1i32 | 3.96E−138 | 0.47586 | 0.814 | 0.371 | 9.85E−134 | A48 | Nr1i3 |
| Fkbp51 | 1.26E−136 | 0.25723 | 0.54 | 0.158 | 3.12E−132 | A48 | Fkbp5 |
| Ephx21 | 6.88E−135 | 0.38687 | 0.993 | 0.919 | 1.71E−130 | A48 | Ephx2 |
| Snord1181 | 9.71E−135 | 0.47712 | 0.766 | 0.324 | 2.41E−130 | A48 | Snord118 |
| Slc27a51 | 3.81E−134 | 0.40618 | 0.987 | 0.885 | 9.45E−130 | A48 | Slc27a5 |
| Ces1b | 1.19E−132 | 0.48295 | 0.838 | 0.445 | 2.95E−128 | A48 | Ces1b |
| Ndrg21 | 1.81E−132 | 0.3482 | 1 | 0.939 | 4.50E−128 | A48 | Ndrg2 |
| Cox8a2 | 3.15E−131 | 0.552 | 0.89 | 0.599 | 7.83E−127 | A48 | Cox8a |
| Cyp2a12 | 1.00E−129 | 0.46643 | 0.981 | 0.879 | 2.49E−125 | A48 | Cyp2a12 |
| Cyp3a252 | 2.18E−129 | 0.36073 | 0.995 | 0.902 | 5.41E−125 | A48 | Cyp3a25 |
| Ambp | 5.70E−128 | 0.28598 | 1 | 0.991 | 1.42E−123 | A48 | Ambp |
| Pabpc1 | 5.17E−127 | 0.4782 | 0.912 | 0.629 | 1.28E−122 | A48 | Pabpc1 |
| Mlycd1 | 9.54E−127 | 0.43784 | 0.893 | 0.5 | 2.37E−122 | A48 | Mlycd |
| F101 | 1.50E−125 | 0.39272 | 0.989 | 0.886 | 3.73E−121 | A48 | F10 |
| Gys21 | 2.76E−124 | 0.42306 | 0.784 | 0.376 | 6.84E−120 | A48 | Gys2 |
| Cdo11 | 1.19E−123 | 0.39707 | 0.997 | 0.956 | 2.96E−119 | A48 | Cdo1 |
| Echs11 | 2.01E−123 | 0.46487 | 0.971 | 0.806 | 5.00E−119 | A48 | Echs1 |
| Bnip32 | 3.06E−123 | 0.46203 | 0.876 | 0.529 | 7.61E−119 | A48 | Bnip3 |
| Slc37a41 | 4.81E−123 | 0.44612 | 0.858 | 0.484 | 1.19E−118 | A48 | Slc37a4 |
| Tmbim61 | 1.33E−122 | 0.37131 | 0.996 | 0.943 | 3.30E−118 | A48 | Tmbim6 |
| Bfar | 3.12E−122 | 0.30801 | 0.742 | 0.306 | 7.75E−118 | A48 | Bfar |
| Ube2r21 | 4.52E−122 | 0.4423 | 0.884 | 0.54 | 1.12E−117 | A48 | Ube2r2 |
| Abcb41 | 2.99E−121 | 0.44252 | 0.956 | 0.688 | 7.42E−117 | A48 | Abcb4 |
| Grn1 | 6.37E−120 | 0.43104 | 0.844 | 0.449 | 1.58E−115 | A48 | Grn |
| Slc25a102 | 3.22E−119 | 0.43127 | 0.877 | 0.526 | 8.00E−115 | A48 | Slc25a10 |
| Hpx1 | 7.59E−118 | 0.38511 | 0.999 | 0.997 | 1.89E−113 | A48 | Hpx |
| Vnn3 | 4.99E−116 | 0.30084 | 0.684 | 0.275 | 1.24E−111 | A48 | Vnn3 |
| Fcgrt1 | 2.63E−115 | 0.40844 | 0.91 | 0.576 | 6.53E−111 | A48 | Fcgrt |
| Fah1 | 8.72E−115 | 0.39129 | 0.974 | 0.821 | 2.17E−110 | A48 | Fah |
| Atp5b | 1.69E−114 | 0.32811 | 0.997 | 0.94 | 4.19E−110 | A48 | Atp5b |
| Amy12 | 4.53E−114 | 0.42158 | 0.943 | 0.66 | 1.13E−109 | A48 | Amy1 |
| Gstt3 | 3.70E−113 | 0.33837 | 0.737 | 0.312 | 9.19E−109 | A48 | Gstt3 |
| Ech11 | 1.29E−112 | 0.41801 | 0.98 | 0.828 | 3.21E−108 | A48 | Ech1 |
| Hsd17b131 | 1.32E−112 | 0.62207 | 0.934 | 0.741 | 3.29E−108 | A48 | Hsd17b13 |
| Serpina3m1 | 6.99E−112 | 0.40339 | 0.949 | 0.749 | 1.74E−107 | A48 | Serpina3m |
| Serpina1d2 | 2.32E−111 | 0.3053 | 0.997 | 0.977 | 5.77E−107 | A48 | Serpina1d |
| Decr21 | 2.53E−109 | 0.41288 | 0.92 | 0.644 | 6.29E−105 | A48 | Decr2 |
| Hspa82 | 5.67E−109 | 0.41074 | 0.952 | 0.731 | 1.41E−104 | A48 | Hspa8 |
| Gltscr2 | 1.30E−108 | 0.34321 | 0.83 | 0.435 | 3.24E−104 | A48 | Gltscr2 |
| Slc25a20 | 2.03E−108 | 0.38754 | 0.877 | 0.508 | 5.03E−104 | A48 | Slc25a20 |
| Abhd14b1 | 5.35E−108 | 0.40591 | 0.885 | 0.554 | 1.33E−103 | A48 | Abhd14b |
| Cebpb2 | 2.85E−107 | 0.36838 | 0.815 | 0.414 | 7.09E−103 | A48 | Cebpb |
| Ahcy1 | 6.37E−107 | 0.33998 | 0.988 | 0.875 | 1.58E−102 | A48 | Ahcy |
| F12 | 9.50E−107 | 0.38801 | 0.966 | 0.803 | 2.36E−102 | A48 | F12 |
| Cox5a1 | 2.15E−106 | 0.41972 | 0.938 | 0.693 | 5.34E−102 | A48 | Cox5a |
| Tcea31 | 3.05E−106 | 0.39376 | 0.825 | 0.448 | 7.58E−102 | A48 | Tcea3 |
| Tmbim41 | 6.73E−106 | 0.36929 | 0.784 | 0.407 | 1.67E−101 | A48 | Tmbim4 |
| Acad11 | 1.16E−105 | 0.38509 | 0.896 | 0.583 | 2.88E−101 | A48 | Acad11 |
| Gabarap1 | 8.50E−105 | 0.40143 | 0.956 | 0.75 | 2.11E−100 | A48 | Gabarap |
| Aldh9a1 | 9.77E−105 | 0.38255 | 0.964 | 0.772 | 2.43E−100 | A48 | Aldh9a1 |
| Ddb1 | 2.16E−103 | 0.35442 | 0.83 | 0.452 | 5.37E−99 | A48 | Ddb1 |
| P4hb2 | 5.46E−103 | 0.29806 | 0.995 | 0.942 | 1.36E−98 | A48 | P4hb |
| Yipf3 | 1.81E−102 | 0.27846 | 0.68 | 0.291 | 4.50E−98 | A48 | Yipf3 |
| Sh3glb1 | 9.56E−102 | 0.30436 | 0.695 | 0.306 | 2.37E−97 | A48 | Sh3glb1 |
| Rpl4 | 2.39E−101 | 0.32886 | 0.987 | 0.912 | 5.93E−97 | A48 | Rpl4 |
| Ctsh1 | 2.95E−101 | 0.42242 | 0.945 | 0.72 | 7.32E−97 | A48 | Ctsh |
| Apoa41 | 1.97E−100 | 0.49471 | 0.778 | 0.4 | 4.90E−96 | A48 | Apoa4 |
| Mgll | 3.72E−100 | 0.36511 | 0.844 | 0.477 | 9.23E−96 | A48 | Mgll |
| Ppara1 | 6.73E−100 | 0.33932 | 0.831 | 0.443 | 1.67E−95 | A48 | Ppara |
| 1600002H07Rik | 1.17E−98 | 0.30158 | 0.736 | 0.353 | 2.91E−94 | A48 | 1600002H07Rik |
| G0s21 | 1.31E−98 | 0.38797 | 0.692 | 0.32 | 3.26E−94 | A48 | G0s2 |
| Cox6a12 | 1.81E−98 | 0.37182 | 0.987 | 0.854 | 4.49E−94 | A48 | Cox6a1 |
| Igfbp4 | 6.46E−98 | 0.33954 | 0.99 | 0.875 | 1.60E−93 | A48 | Igfbp4 |
| C4b1 | 2.60E−97 | 0.39891 | 0.975 | 0.876 | 6.46E−93 | A48 | C4b |
| Sdhc | 2.46E−96 | 0.3865 | 0.91 | 0.645 | 6.10E−92 | A48 | Sdhc |
| Apoc42 | 3.94E−96 | 0.40964 | 0.998 | 0.965 | 9.79E−92 | A48 | Apoc4 |
| Serpinf1 | 4.30E−96 | 0.35652 | 0.976 | 0.855 | 1.07E−91 | A48 | Serpinf1 |
| Wbp1l1 | 7.44E−96 | 0.38751 | 0.91 | 0.648 | 1.85E−91 | A48 | Wbp1l |
| Aldoa | 1.89E−95 | 0.27774 | 0.719 | 0.329 | 4.70E−91 | A48 | Aldoa |
| Atp5k1 | 2.67E−95 | 0.38427 | 0.935 | 0.664 | 6.63E−91 | A48 | Atp5k |
| Aspdh | 5.69E−95 | 0.32171 | 0.776 | 0.397 | 1.41E−90 | A48 | Aspdh |
| Gm2a | 4.55E−94 | 0.34919 | 0.827 | 0.459 | 1.13E−89 | A48 | Gm2a |
| Etnppl2 | 1.17E−93 | 0.52531 | 0.866 | 0.587 | 2.89E−89 | A48 | Etnppl |
| Etfb1 | 8.54E−93 | 0.30917 | 0.996 | 0.928 | 2.12E−88 | A48 | Etfb |
| Sec14l21 | 1.30E−92 | 0.35236 | 0.98 | 0.835 | 3.23E−88 | A48 | Sec14l2 |
| Camk2n11 | 2.32E−92 | 0.27301 | 0.691 | 0.315 | 5.77E−88 | A48 | Camk2n1 |
| Slc10a11 | 2.86E−92 | 0.37533 | 0.984 | 0.84 | 7.10E−88 | A48 | Slc10a1 |
| Ifnar2 | 6.13E−92 | 0.27491 | 0.731 | 0.34 | 1.52E−87 | A48 | Ifnar2 |
| Cpt2 | 8.07E−92 | 0.32669 | 0.807 | 0.437 | 2.00E−87 | A48 | Cpt2 |
| Azgp1 | 2.23E−91 | 0.25201 | 0.997 | 0.966 | 5.53E−87 | A48 | Azgp1 |
| Uroc1 | 1.35E−90 | 0.40743 | 0.914 | 0.637 | 3.34E−86 | A48 | Uroc1 |
| Pbld11 | 6.93E−90 | 0.34533 | 0.881 | 0.566 | 1.72E−85 | A48 | Pbld1 |
| Brap | 1.06E−89 | 0.31593 | 0.829 | 0.465 | 2.62E−85 | A48 | Brap |
| Shmt11 | 1.20E−89 | 0.34796 | 0.882 | 0.556 | 2.98E−85 | A48 | Shmt1 |
| Slc27a2 | 1.39E−89 | 0.31391 | 0.996 | 0.945 | 3.44E−85 | A48 | Slc27a2 |
| Asgr21 | 7.88E−89 | 0.34851 | 0.879 | 0.552 | 1.96E−84 | A48 | Asgr2 |
| Aco2 | 3.32E−88 | 0.32671 | 0.846 | 0.507 | 8.24E−84 | A48 | Aco2 |
| Ctsd | 1.28E−87 | 0.35447 | 0.916 | 0.642 | 3.17E−83 | A48 | Ctsd |
| Cyp3a112 | 3.21E−87 | 0.39704 | 1 | 0.995 | 7.98E−83 | A48 | Cyp3a11 |
| Sdhd2 | 4.40E−87 | 0.43472 | 0.891 | 0.655 | 1.09E−82 | A48 | Sdhd |
| 0610005C13Rik1 | 6.63E−86 | 0.33556 | 0.944 | 0.665 | 1.65E−81 | A48 | 0610005C13Rik |
| Slc40a11 | 9.34E−86 | 0.27255 | 0.744 | 0.367 | 2.32E−81 | A48 | Slc40a1 |
| Cyp2c441 | 1.12E−85 | 0.41494 | 0.924 | 0.692 | 2.77E−81 | A48 | Cyp2c44 |
| Lamp11 | 1.61E−85 | 0.32739 | 0.982 | 0.877 | 4.01E−81 | A48 | Lamp1 |
| Creb3l3 | 5.60E−85 | 0.33049 | 0.971 | 0.818 | 1.39E−80 | A48 | Creb3l3 |
| Pigr | 1.16E−84 | 0.40014 | 0.977 | 0.945 | 2.89E−80 | A48 | Pigr |
| Apoc12 | 4.87E−84 | 0.29282 | 1 | 0.992 | 1.21E−79 | A48 | Apoc1 |
| Smlr11 | 5.67E−84 | 0.25158 | 0.68 | 0.317 | 1.41E−79 | A48 | Smlr1 |
| Snx3 | 7.63E−84 | 0.2563 | 0.693 | 0.331 | 1.89E−79 | A48 | Snx3 |
| Atp5g31 | 1.55E−83 | 0.32884 | 0.985 | 0.888 | 3.85E−79 | A48 | Atp5g3 |
| Cyp2d26 | 4.14E−83 | 0.30505 | 0.982 | 0.897 | 1.03E−78 | A48 | Cyp2d26 |
| Klf9 | 4.69E−83 | 0.33261 | 0.862 | 0.549 | 1.17E−78 | A48 | Klf9 |
| Eci2 | 5.28E−83 | 0.32811 | 0.944 | 0.697 | 1.31E−78 | A48 | Eci2 |
| Slc2a21 | 7.42E−83 | 0.35696 | 0.824 | 0.485 | 1.84E−78 | A48 | Slc2a2 |
| Acot12 | 6.96E−82 | 0.27848 | 0.825 | 0.454 | 1.73E−77 | A48 | Acot12 |
| Tmem120a | 1.04E−81 | 0.2525 | 0.708 | 0.343 | 2.59E−77 | A48 | Tmem120a |
| Pglyrp2 | 6.41E−81 | 0.30742 | 0.754 | 0.402 | 1.59E−76 | A48 | Pglyrp2 |
| Abcc3 | 1.62E−80 | 0.35577 | 0.859 | 0.549 | 4.03E−76 | A48 | Abcc3 |
| Fam195a1 | 4.52E−80 | 0.28611 | 0.726 | 0.359 | 1.12E−75 | A48 | Fam195a |
| Ctsa | 8.45E−80 | 0.25496 | 0.731 | 0.36 | 2.10E−75 | A48 | Ctsa |
| Oplah | 1.28E−79 | 0.30336 | 0.805 | 0.457 | 3.19E−75 | A48 | Oplah |
| Acat11 | 4.32E−79 | 0.30733 | 0.978 | 0.842 | 1.07E−74 | A48 | Acat1 |
| Park71 | 3.18E−78 | 0.34371 | 0.953 | 0.774 | 7.89E−74 | A48 | Park7 |
| Pbld2 | 4.10E−78 | 0.31208 | 0.891 | 0.58 | 1.02E−73 | A48 | Pbld2 |
| Serpina3n1 | 9.63E−78 | 0.31814 | 0.902 | 0.626 | 2.39E−73 | A48 | Serpina3n |
| Serinc31 | 3.42E−77 | 0.28974 | 0.793 | 0.452 | 8.48E−73 | A48 | Serinc3 |
| Pitpnb | 8.76E−77 | 0.26612 | 0.795 | 0.431 | 2.18E−72 | A48 | Pitpnb |
| Usmg52 | 9.41E−77 | 0.37613 | 0.755 | 0.419 | 2.34E−72 | A48 | Usmg5 |
| Ninj11 | 9.74E−77 | 0.34623 | 0.874 | 0.587 | 2.42E−72 | A48 | Ninj1 |
| Hsd17b102 | 2.43E−76 | 0.40473 | 0.927 | 0.686 | 6.03E−72 | A48 | Hsd17b10 |
| Eef1d | 6.80E−75 | 0.2572 | 0.795 | 0.434 | 1.69E−70 | A48 | Eef1d |
| Lipa1 | 2.40E−74 | 0.31862 | 0.904 | 0.63 | 5.96E−70 | A48 | Lipa |
| Agpat6 | 3.14E−74 | 0.28948 | 0.884 | 0.558 | 7.81E−70 | A48 | Agpat6 |
| Pim31 | 3.82E−74 | 0.31374 | 0.845 | 0.52 | 9.48E−70 | A48 | Pim3 |
| Pcx | 5.84E−74 | 0.31598 | 0.912 | 0.66 | 1.45E−69 | A48 | Pcx |
| Creg11 | 3.96E−73 | 0.29796 | 0.993 | 0.917 | 9.83E−69 | A48 | Creg1 |
| Gcdh1 | 4.36E−73 | 0.28849 | 0.977 | 0.848 | 1.08E−68 | A48 | Gcdh |
| Atp5d1 | 1.54E−72 | 0.30761 | 0.935 | 0.694 | 3.81E−68 | A48 | Atp5d |
| Gfra11 | 1.91E−72 | 0.28896 | 0.867 | 0.539 | 4.74E−68 | A48 | Gfra1 |
| Pck11 | 2.03E−72 | 0.35008 | 0.999 | 0.974 | 5.03E−68 | A48 | Pck1 |
| Gstt2 | 1.93E−71 | 0.25927 | 0.754 | 0.398 | 4.80E−67 | A48 | Gstt2 |
| Sord1 | 3.59E−71 | 0.30187 | 0.987 | 0.917 | 8.92E−67 | A48 | Sord |
| Tmem371 | 7.54E−71 | 0.26106 | 0.786 | 0.429 | 1.87E−66 | A48 | Tmem37 |
| Pla2g12b | 8.56E−71 | 0.27883 | 0.798 | 0.457 | 2.13E−66 | A48 | Pla2g12b |
| Ndufb22 | 1.82E−70 | 0.3484 | 0.888 | 0.589 | 4.53E−66 | A48 | Ndufb2 |
| Atf5 | 2.73E−70 | 0.31719 | 0.909 | 0.649 | 6.78E−66 | A48 | Atf5 |
| Sfxn1 | 2.87E−70 | 0.32927 | 0.822 | 0.522 | 7.13E−66 | A48 | Sfxn1 |
| Glrx52 | 3.18E−70 | 0.29996 | 0.886 | 0.568 | 7.89E−66 | A48 | Glrx5 |
| Atp5j1 | 2.90E−69 | 0.35068 | 0.916 | 0.73 | 7.19E−65 | A48 | Atp5j |
| Ggnbp2 | 3.08E−69 | 0.25778 | 0.818 | 0.458 | 7.65E−65 | A48 | Ggnbp2 |
| Furin | 4.55E−69 | 0.28056 | 0.781 | 0.445 | 1.13E−64 | A48 | Furin |
| Ndufb101 | 7.41E−69 | 0.31642 | 0.907 | 0.669 | 1.84E−64 | A48 | Ndufb10 |
| Dhrs41 | 1.15E−68 | 0.27653 | 0.94 | 0.671 | 2.87E−64 | A48 | Dhrs4 |
| Sec61a1 | 1.50E−68 | 0.28991 | 0.825 | 0.513 | 3.72E−64 | A48 | Sec61a1 |
| Zfp91 | 1.51E−68 | 0.27537 | 0.855 | 0.531 | 3.75E−64 | A48 | Zfp91 |
| D17Wsu92e | 1.81E−68 | 0.28265 | 0.909 | 0.62 | 4.49E−64 | A48 | D17Wsu92e |
| Sult1d12 | 2.07E−68 | 0.4399 | 0.936 | 0.68 | 5.14E−64 | A48 | Sult1d1 |
| Mst1 | 3.99E−68 | 0.27754 | 0.81 | 0.489 | 9.91E−64 | A48 | Mst1 |
| Igf1 | 7.06E−68 | 0.30268 | 0.985 | 0.902 | 1.75E−63 | A48 | Igf1 |
| Acadvl | 8.03E−68 | 0.2803 | 0.959 | 0.782 | 1.99E−63 | A48 | Acadvl |
| Hgfac | 1.41E−67 | 0.26562 | 0.786 | 0.457 | 3.49E−63 | A48 | Hgfac |
| 11-Mar | 1.82E−67 | 0.28124 | 0.984 | 0.869 | 4.51E−63 | A48 | 1-Mar |
| Tgoln1 | 4.48E−67 | 0.30552 | 0.891 | 0.602 | 1.11E−62 | A48 | Tgoln1 |
| Upb11 | 4.61E−67 | 0.30588 | 0.945 | 0.746 | 1.14E−62 | A48 | Upb1 |
| Acbd51 | 7.35E−67 | 0.28409 | 0.943 | 0.711 | 1.82E−62 | A48 | Acbd5 |
| Sds2 | 2.26E−66 | 0.55395 | 0.799 | 0.612 | 5.62E−62 | A48 | Sds |
| Map2k2 | 2.40E−66 | 0.25507 | 0.832 | 0.517 | 5.96E−62 | A48 | Map2k2 |
| Ndufs71 | 2.84E−66 | 0.25451 | 0.847 | 0.51 | 7.05E−62 | A48 | Ndufs7 |
| Dcxr2 | 3.22E−66 | 0.29887 | 0.884 | 0.591 | 7.99E−62 | A48 | Dcxr |
| Gpt1 | 3.35E−66 | 0.2881 | 0.94 | 0.737 | 8.32E−62 | A48 | Gpt |
| Gpld1 | 6.68E−66 | 0.28871 | 0.877 | 0.595 | 1.66E−61 | A48 | Gpld1 |
| 1100001G20Rik1 | 7.71E−66 | 0.34154 | 0.993 | 0.968 | 1.91E−61 | A48 | 1100001G20Rik |
| Ndufs81 | 2.39E−65 | 0.31488 | 0.854 | 0.581 | 5.94E−61 | A48 | Ndufs8 |
| Abca6 | 5.35E−65 | 0.28403 | 0.849 | 0.548 | 1.33E−60 | A48 | Abca6 |
| Actn4 | 4.59E−64 | 0.25336 | 0.789 | 0.465 | 1.14E−59 | A48 | Actn4 |
| Gchfr2 | 2.18E−63 | 0.32395 | 0.953 | 0.727 | 5.42E−59 | A48 | Gchfr |
| Mia3 | 6.64E−63 | 0.28475 | 0.91 | 0.64 | 1.65E−58 | A48 | Mia3 |
| Cd1d11 | 8.52E−63 | 0.29268 | 0.887 | 0.611 | 2.11E−58 | A48 | Cd1d1 |
| Tmem176b1 | 9.86E−63 | 0.28825 | 0.89 | 0.63 | 2.45E−58 | A48 | Tmem176b |
| Lpin21 | 1.01E−62 | 0.30413 | 0.921 | 0.653 | 2.52E−58 | A48 | Lpin2 |
| Pccb | 1.17E−62 | 0.28017 | 0.901 | 0.642 | 2.90E−58 | A48 | Pccb |
| Cideb2 | 1.52E−62 | 0.31595 | 0.892 | 0.65 | 3.79E−58 | A48 | Cideb |
| Itih1 | 2.19E−62 | 0.28878 | 0.974 | 0.819 | 5.45E−58 | A48 | Itih1 |
| B2m2 | 2.44E−62 | 0.25723 | 0.997 | 0.949 | 6.06E−58 | A48 | B2m |
| Maob | 5.02E−62 | 0.28848 | 0.889 | 0.597 | 1.25E−57 | A48 | Maob |
| Slc25a39 | 6.32E−62 | 0.29589 | 0.938 | 0.762 | 1.57E−57 | A48 | Slc25a39 |
| Acadl | 9.46E−62 | 0.2603 | 0.916 | 0.643 | 2.35E−57 | A48 | Acadl |
| Hadh1 | 1.41E−61 | 0.27999 | 0.908 | 0.641 | 3.49E−57 | A48 | Hadh |
| Akr1c191 | 5.83E−61 | 0.29518 | 0.821 | 0.507 | 1.45E−56 | A48 | Akr1c19 |
| Os9 | 1.17E−60 | 0.26825 | 0.911 | 0.673 | 2.90E−56 | A48 | Os9 |
| Pemt1 | 1.39E−60 | 0.31675 | 0.948 | 0.754 | 3.46E−56 | A48 | Pemt |
| Hsbp11 | 1.15E−59 | 0.25477 | 0.792 | 0.495 | 2.86E−55 | A48 | Hsbp1 |
| Serpinf2 | 2.36E−59 | 0.26266 | 0.966 | 0.847 | 5.86E−55 | A48 | Serpinf2 |
| Ndufa131 | 6.13E−59 | 0.28644 | 0.959 | 0.769 | 1.52E−54 | A48 | Ndufa13 |
| Gsto1 | 6.52E−59 | 0.27994 | 0.818 | 0.518 | 1.62E−54 | A48 | Gsto1 |
| Ubxn4 | 2.22E−58 | 0.25605 | 0.893 | 0.601 | 5.52E−54 | A48 | Ubxn4 |
| Slc25a13 | 2.47E−58 | 0.26362 | 0.924 | 0.668 | 6.14E−54 | A48 | Slc25a13 |
| Hadha | 3.06E−58 | 0.279 | 0.943 | 0.743 | 7.60E−54 | A48 | Hadha |
| Surf4 | 3.88E−58 | 0.25226 | 0.873 | 0.578 | 9.64E−54 | A48 | Surf4 |
| Hal1 | 4.19E−58 | 0.38266 | 0.908 | 0.741 | 1.04E−53 | A48 | Hal |
| Akr1d11 | 1.29E−57 | 0.2648 | 0.852 | 0.543 | 3.22E−53 | A48 | Akr1d1 |
| Rabac12 | 5.41E−56 | 0.25087 | 0.807 | 0.498 | 1.34E−51 | A48 | Rabac1 |
| Tspo1 | 7.02E−56 | 0.26601 | 0.784 | 0.477 | 1.74E−51 | A48 | Tspo |
| Mttp | 9.90E−56 | 0.2617 | 0.933 | 0.709 | 2.46E−51 | A48 | Mttp |
| Ctage5 | 2.71E−55 | 0.27451 | 0.941 | 0.698 | 6.74E−51 | A48 | Ctage5 |
| Agpat3 | 3.06E−55 | 0.2526 | 0.874 | 0.617 | 7.61E−51 | A48 | Agpat3 |
| Gamt1 | 3.37E−55 | 0.26806 | 0.841 | 0.564 | 8.36E−51 | A48 | Gamt |
| Dgat21 | 3.63E−55 | 0.2637 | 0.947 | 0.753 | 9.02E−51 | A48 | Dgat2 |
| Sardh | 7.35E−53 | 0.27838 | 0.959 | 0.829 | 1.82E−48 | A48 | Sardh |
| Asl1 | 2.33E−51 | 0.33611 | 0.953 | 0.801 | 5.79E−47 | A48 | Asl |
| Acsl12 | 5.59E−51 | 0.25453 | 0.978 | 0.895 | 1.39E−46 | A48 | Acsl1 |
| Ccs1 | 9.34E−50 | 0.26432 | 0.895 | 0.655 | 2.32E−45 | A48 | Ccs |
| BC089597 | 1.42E−49 | 0.25099 | 0.813 | 0.516 | 3.52E−45 | A48 | BC089597 |
| Por2 | 5.03E−49 | 0.28933 | 0.921 | 0.688 | 1.25E−44 | A48 | Por |
| Uqcrfs1 | 6.79E−48 | 0.25793 | 0.949 | 0.781 | 1.69E−43 | A48 | Uqcrfs1 |
| Hagh1 | 4.03E−47 | 0.28738 | 0.959 | 0.834 | 1.00E−42 | A48 | Hagh |
| Tmem14c1 | 5.76E−47 | 0.25067 | 0.837 | 0.561 | 1.43E−42 | A48 | Tmem14c |
| Dak | 5.90E−47 | 0.29663 | 0.852 | 0.575 | 1.47E−42 | A48 | Dak |
| Leap22 | 2.60E−44 | 0.33563 | 0.833 | 0.576 | 6.45E−40 | A48 | Leap2 |
| Cpt1a | 1.94E−43 | 0.26499 | 0.896 | 0.681 | 4.81E−39 | A48 | Cpt1a |
| Mpc22 | 1.37E−42 | 0.28745 | 0.953 | 0.826 | 3.40E−38 | A48 | Mpc2 |
| Cd3021 | 6.25E−40 | 0.27124 | 0.956 | 0.844 | 1.55E−35 | A48 | Cd302 |
| Rps27l2 | 8.74E−39 | 0.28686 | 0.924 | 0.795 | 2.17E−34 | A48 | Rps27l |
| Cyp2f21 | 3.99E−31 | 0.29608 | 0.912 | 0.833 | 9.91E−27 | A48 | Cyp2f2 |
| Slc7a2 | 3.98E−30 | 0.25021 | 0.921 | 0.758 | 9.87E−26 | A48 | Slc7a2 |
| Glul | 2.05E−13 | 0.54015 | 0.308 | 0.18 | 5.09E−09 | A48 | Glul |
| Apoc13 | 1.14E−259 | 0.54953 | 1 | 0.992 | 2.84E−255 | A96 | Apoc1 |
| Cyp4a143 | 2.66E−250 | 0.97024 | 0.975 | 0.511 | 6.60E−246 | A96 | Cyp4a14 |
| Cyp4a102 | 2.15E−216 | 0.86815 | 0.974 | 0.651 | 5.34E−212 | A96 | Cyp4a10 |
| Mat1a3 | 3.10E−210 | 0.59668 | 1 | 0.97 | 7.70E−206 | A96 | Mat1a |
| Angptl33 | 5.10E−206 | 0.56056 | 0.998 | 0.946 | 1.27E−201 | A96 | Angptl3 |
| Cdo12 | 3.07E−182 | 0.50859 | 0.998 | 0.957 | 7.61E−178 | A96 | Cdo1 |
| Apoa22 | 2.85E−177 | 0.34475 | 1 | 0.999 | 7.07E−173 | A96 | Apoa2 |
| Zfp683 | 2.01E−176 | 0.41844 | 0.476 | 0.092 | 4.99E−172 | A96 | Zfp683 |
| Apoe3 | 2.87E−148 | 0.31614 | 1 | 1 | 7.13E−144 | A96 | Apoe |
| Chchd103 | 1.38E−138 | 0.47684 | 0.994 | 0.927 | 3.43E−134 | A96 | Chchd10 |
| Ttr3 | 2.28E−137 | 0.31745 | 1 | 0.999 | 5.66E−133 | A96 | Ttr |
| Ttpa1 | 4.82E−129 | 0.49263 | 0.98 | 0.879 | 1.20E−124 | A96 | Ttpa |
| Scd12 | 2.34E−118 | 0.52026 | 0.989 | 0.926 | 5.82E−114 | A96 | Scd1 |
| Ubc2 | 2.86E−109 | 0.48097 | 0.968 | 0.839 | 7.10E−105 | A96 | Ubc |
| Apoh3 | 2.32E−107 | 0.30846 | 0.998 | 0.979 | 5.76E−103 | A96 | Apoh |
| Ces1d1 | 9.98E−105 | 0.45085 | 0.977 | 0.905 | 2.48E−100 | A96 | Ces1d |
| Apoc33 | 1.84E−102 | 0.33815 | 1 | 0.978 | 4.56E−98 | A96 | Apoc3 |
| Ces1c3 | 8.24E−97 | 0.29829 | 1 | 0.985 | 2.05E−92 | A96 | Ces1e |
| Hmgcs23 | 2.29E−95 | 0.30409 | 0.996 | 0.977 | 5.70E−91 | A96 | Hmgcs2 |
| Slco1b21 | 3.01E−92 | 0.51568 | 0.998 | 0.929 | 7.48E−88 | A96 | Slco1b2 |
| Cyp8b12 | 6.15E−88 | 0.51669 | 0.897 | 0.691 | 1.53E−83 | A96 | Cyp8b1 |
| Hamp2 | 4.13E−86 | 0.54886 | 0.92 | 0.788 | 1.03E−81 | A96 | Hamp |
| Slc27a21 | 7.10E−80 | 0.32514 | 0.988 | 0.947 | 1.76E−75 | A96 | Slc27a2 |
| Hspa83 | 1.35E−77 | 0.41417 | 0.905 | 0.741 | 3.34E−73 | A96 | Hspa8 |
| Mettl7b1 | 3.18E−76 | 0.37013 | 0.949 | 0.866 | 7.90E−72 | A96 | Mettl7b |
| Cyp3a113 | 1.94E−75 | 0.275 | 0.999 | 0.995 | 4.82E−71 | A96 | Cyp3a11 |
| Crot1 | 7.79E−75 | 0.31433 | 0.989 | 0.942 | 1.94E−70 | A96 | Crot |
| Cyp2c292 | 1.44E−68 | 0.55102 | 0.962 | 0.85 | 3.59E−64 | A96 | Cyp2c29 |
| Acbd52 | 3.67E−64 | 0.3903 | 0.87 | 0.725 | 9.12E−60 | A96 | Acbd5 |
| Acsl13 | 2.22E−63 | 0.3297 | 0.977 | 0.897 | 5.52E−59 | A96 | Acsl1 |
| Sult1a12 | 2.88E−62 | 0.38498 | 0.881 | 0.733 | 7.15E−58 | A96 | Sult1a1 |
| Aldh2 | 6.54E−62 | 0.28252 | 0.995 | 0.968 | 1.62E−57 | A96 | Aldh2 |
| Retsat3 | 2.39E−61 | 0.36555 | 0.957 | 0.834 | 5.92E−57 | A96 | Retsat |
| Abcb42 | 3.19E−61 | 0.38995 | 0.878 | 0.704 | 7.92E−57 | A96 | Abcb4 |
| Ndrg22 | 1.13E−60 | 0.27001 | 0.985 | 0.942 | 2.81E−56 | A96 | Ndrg2 |
| Aldh3a22 | 3.23E−60 | 0.56589 | 0.687 | 0.451 | 8.02E−56 | A96 | Aldh3a2 |
| Atp5k2 | 9.30E−60 | 0.37147 | 0.837 | 0.682 | 2.31E−55 | A96 | Atp5k |
| Amy13 | 2.54E−58 | 0.38615 | 0.831 | 0.681 | 6.31E−54 | A96 | Amy1 |
| Igfbp41 | 2.23E−56 | 0.29578 | 0.964 | 0.881 | 5.53E−52 | A96 | Igfbp4 |
| Chpt11 | 2.52E−56 | 0.3802 | 0.848 | 0.694 | 6.27E−52 | A96 | Chpt1 |
| Pex11a1 | 4.35E−56 | 0.44625 | 0.7 | 0.484 | 1.08E−51 | A96 | Pex11a |
| Hhex1 | 7.20E−54 | 0.35579 | 0.543 | 0.313 | 1.79E−49 | A96 | Hhex |
| Sult1d13 | 2.09E−51 | 0.38992 | 0.855 | 0.696 | 5.20E−47 | A96 | Sult1d1 |
| Atp5h3 | 1.30E−50 | 0.31445 | 0.848 | 0.657 | 3.24E−46 | A96 | Atp5h |
| Cyp3a253 | 5.89E−50 | 0.2703 | 0.981 | 0.905 | 1.46E−45 | A96 | Cyp3a25 |
| Lipa2 | 1.77E−49 | 0.35594 | 0.791 | 0.651 | 4.41E−45 | A96 | Lipa |
| Cyp2e12 | 4.42E−49 | 0.51183 | 0.93 | 0.809 | 1.10E−44 | A96 | Cyp2e1 |
| Prlr | 2.34E−48 | 0.34641 | 0.574 | 0.359 | 5.82E−44 | A96 | Prlr |
| Clpx1 | 3.44E−48 | 0.3254 | 0.884 | 0.764 | 8.54E−44 | A96 | Clpx |
| Tmem56 | 4.52E−47 | 0.33425 | 0.812 | 0.643 | 1.12E−42 | A96 | Tmem56 |
| Insig22 | 1.46E−46 | 0.28539 | 0.928 | 0.818 | 3.63E−42 | A96 | Insig2 |
| Hrg2 | 6.03E−46 | 0.28054 | 0.923 | 0.845 | 1.50E−41 | A96 | Hrg |
| Ube2r22 | 1.09E−45 | 0.33818 | 0.747 | 0.565 | 2.70E−41 | A96 | Ube2r2 |
| Ppp2r5a1 | 3.21E−45 | 0.35054 | 0.764 | 0.615 | 7.96E−41 | A96 | Ppp2r5a |
| Nr1i33 | 6.04E−44 | 0.34813 | 0.625 | 0.405 | 1.50E−39 | A96 | Nr1i3 |
| Cox6a13 | 9.03E−44 | 0.27455 | 0.943 | 0.862 | 2.24E−39 | A96 | Cox6a1 |
| Creg12 | 1.75E−43 | 0.25169 | 0.985 | 0.919 | 4.34E−39 | A96 | Creg1 |
| Acaa1b2 | 3.05E−43 | 0.28198 | 0.986 | 0.919 | 7.56E−39 | A96 | Acaa1b |
| Cyp7a1 | 1.08E−42 | 0.37883 | 0.398 | 0.199 | 2.68E−38 | A96 | Cyp7a1 |
| Cyp2c542 | 1.49E−42 | 0.34469 | 0.86 | 0.726 | 3.70E−38 | A96 | Cyp2c54 |
| Cyp2c502 | 4.24E−42 | 0.32419 | 0.943 | 0.847 | 1.05E−37 | A96 | Cyp2c50 |
| Snord1182 | 9.05E−42 | 0.37824 | 0.564 | 0.36 | 2.25E−37 | A96 | Snord118 |
| Asl2 | 9.71E−42 | 0.27504 | 0.896 | 0.811 | 2.41E−37 | A96 | Asl |
| Csad2 | 9.95E−42 | 0.42065 | 0.843 | 0.684 | 2.47E−37 | A96 | Csad |
| Sc4mol2 | 2.31E−41 | 0.33609 | 0.781 | 0.631 | 5.74E−37 | A96 | Sc4mol |
| Igfbp13 | 2.40E−41 | 0.26025 | 0.826 | 0.691 | 5.95E−37 | A96 | Igfbp1 |
| Gabarapl11 | 6.61E−41 | 0.29324 | 0.881 | 0.78 | 1.64E−36 | A96 | Gabarapl1 |
| Tsc22d3 | 8.65E−41 | 0.33625 | 0.534 | 0.338 | 2.15E−36 | A96 | Tsc22d3 |
| Acot11 | 1.16E−40 | 0.31059 | 0.459 | 0.257 | 2.88E−36 | A96 | Acot1 |
| Gyk | 2.01E−40 | 0.3134 | 0.616 | 0.441 | 5.00E−36 | A96 | Gyk |
| Decr22 | 3.24E−40 | 0.30175 | 0.813 | 0.664 | 8.03E−36 | A96 | Decr2 |
| Cth1 | 3.77E−40 | 0.29664 | 0.944 | 0.826 | 9.37E−36 | A96 | Cth |
| Pon12 | 4.32E−40 | 0.28987 | 0.965 | 0.888 | 1.07E−35 | A96 | Pon1 |
| Cd36 | 9.83E−40 | 0.31288 | 0.299 | 0.131 | 2.44E−35 | A96 | Cd36 |
| Aplp2 | 1.81E−39 | 0.29028 | 0.826 | 0.67 | 4.48E−35 | A96 | Aplp2 |
| Agpat61 | 2.42E−38 | 0.31428 | 0.743 | 0.584 | 6.01E−34 | A96 | Agpat6 |
| Agt2 | 2.70E−38 | 0.25501 | 0.912 | 0.827 | 6.71E−34 | A96 | Agt |
| Hmgcl3 | 1.80E−37 | 0.27438 | 0.871 | 0.739 | 4.47E−33 | A96 | Hmgcl |
| Gstt31 | 3.07E−37 | 0.30258 | 0.541 | 0.347 | 7.61E−33 | A96 | Gstt3 |
| Aqp91 | 3.47E−37 | 0.31777 | 0.801 | 0.664 | 8.62E−33 | A96 | Aqp9 |
| Akr1d12 | 6.11E−37 | 0.29936 | 0.721 | 0.567 | 1.52E−32 | A96 | Akr1d1 |
| Slc4a41 | 7.42E−37 | 0.29765 | 0.609 | 0.42 | 1.84E−32 | A96 | Slc4a4 |
| Ptms1 | 8.05E−37 | 0.28625 | 0.806 | 0.71 | 2.00E−32 | A96 | Ptms |
| Rgn2 | 9.16E−37 | 0.36863 | 0.899 | 0.786 | 2.27E−32 | A96 | Rgn |
| Lcat2 | 9.19E−37 | 0.26158 | 0.886 | 0.786 | 2.28E−32 | A96 | Leaf |
| Hpgd1 | 1.45E−36 | 0.34118 | 0.588 | 0.41 | 3.59E−32 | A96 | Hpgd |
| Hmgcs11 | 1.88E−36 | 0.32515 | 0.858 | 0.718 | 4.67E−32 | A96 | Hmgcs1 |
| Rdh16 | 2.69E−36 | 0.34767 | 0.494 | 0.31 | 6.69E−32 | A96 | Rdh16 |
| Cyp2c372 | 2.78E−36 | 0.41436 | 0.71 | 0.521 | 6.89E−32 | A96 | Cyp2c37 |
| Tob11 | 1.03E−35 | 0.29582 | 0.649 | 0.487 | 2.55E−31 | A96 | Tobi |
| Kif1b | 2.07E−35 | 0.2961 | 0.686 | 0.507 | 5.15E−31 | A96 | Kif1b |
| Bnip33 | 3.84E−35 | 0.32084 | 0.716 | 0.558 | 9.53E−31 | A96 | Bnip3 |
| Ndufa31 | 4.91E−35 | 0.27429 | 0.737 | 0.598 | 1.22E−30 | A96 | Ndufa3 |
| Apoc22 | 5.09E−35 | 0.27811 | 0.901 | 0.815 | 1.26E−30 | A96 | Apoc2 |
| Aldh9a11 | 7.77E−35 | 0.25848 | 0.884 | 0.786 | 1.93E−30 | A96 | Aldh9a1 |
| Cpt1a1 | 4.44E−34 | 0.26256 | 0.837 | 0.693 | 1.10E−29 | A96 | Cpt1a |
| Hsd17b7 | 7.40E−34 | 0.30279 | 0.56 | 0.386 | 1.84E−29 | A96 | Hsd17b7 |
| Acat12 | 7.72E−34 | 0.2558 | 0.917 | 0.852 | 1.92E−29 | A96 | Acat1 |
| D4Wsu53e | 8.98E−34 | 0.29281 | 0.597 | 0.413 | 2.23E−29 | A96 | D4Wsu53e |
| Cox5a2 | 9.78E−34 | 0.27283 | 0.813 | 0.715 | 2.43E−29 | A96 | Cox5a |
| Gsta21 | 1.28E−33 | 0.31098 | 0.522 | 0.356 | 3.17E−29 | A96 | Gsta2 |
| Cxcl121 | 1.54E−33 | 0.28215 | 0.635 | 0.48 | 3.82E−29 | A96 | Cxcl12 |
| Baat1 | 1.66E−33 | 0.27278 | 0.862 | 0.769 | 4.13E−29 | A96 | Baat |
| Cyp2c681 | 1.69E−33 | 0.29308 | 0.695 | 0.527 | 4.20E−29 | A96 | Cyp2c68 |
| Hsd17b103 | 4.80E−33 | 0.2802 | 0.841 | 0.702 | 1.19E−28 | A96 | Hsd17b10 |
| Ndufa72 | 7.78E−33 | 0.26 | 0.818 | 0.695 | 1.93E−28 | A96 | Ndufa7 |
| Rdx2 | 1.06E−32 | 0.25662 | 0.824 | 0.683 | 2.63E−28 | A96 | Rdx |
| Gas51 | 1.39E−32 | 0.28228 | 0.68 | 0.525 | 3.46E−28 | A96 | Gas5 |
| G6pc1 | 2.86E−32 | 0.25415 | 0.86 | 0.728 | 7.09E−28 | A96 | G6pc |
| Cpeb4 | 3.54E−32 | 0.3011 | 0.527 | 0.357 | 8.79E−28 | A96 | Cpeb4 |
| Tgoln11 | 3.58E−32 | 0.28089 | 0.756 | 0.626 | 8.89E−28 | A96 | Tgoln1 |
| Tmed51 | 4.26E−32 | 0.25232 | 0.714 | 0.569 | 1.06E−27 | A96 | Tmed5 |
| Por3 | 4.35E−32 | 0.28673 | 0.858 | 0.701 | 1.08E−27 | A96 | Por |
| Abcc31 | 9.20E−32 | 0.27433 | 0.733 | 0.572 | 2.28E−27 | A96 | Abcc3 |
| Etfdh | 9.29E−32 | 0.26388 | 0.811 | 0.706 | 2.31E−27 | A96 | Etfdh |
| Arl4a | 1.24E−31 | 0.27062 | 0.517 | 0.342 | 3.09E−27 | A96 | Arl4a |
| Cpox | 1.57E−31 | 0.32292 | 0.634 | 0.486 | 3.91E−27 | A96 | Cpox |
| Zfp36l1 | 2.69E−31 | 0.29635 | 0.601 | 0.447 | 6.69E−27 | A96 | Zfp36l1 |
| Dhdh1 | 2.78E−31 | 0.26256 | 0.734 | 0.615 | 6.90E−27 | A96 | Dhdh |
| Slco1a41 | 5.51E−31 | 0.30491 | 0.507 | 0.336 | 1.37E−26 | A96 | Slco1a4 |
| Cyp2c442 | 1.06E−30 | 0.27354 | 0.828 | 0.71 | 2.64E−26 | A96 | Cyp2c44 |
| Ctage51 | 1.29E−29 | 0.25352 | 0.837 | 0.716 | 3.22E−25 | A96 | Ctage5 |
| Mia31 | 2.42E−29 | 0.26045 | 0.81 | 0.659 | 6.02E−25 | A96 | Mia3 |
| Slc25a201 | 3.50E−29 | 0.25073 | 0.671 | 0.543 | 8.69E−25 | A96 | Slc25a20 |
| G0s22 | 7.31E−29 | 0.26556 | 0.525 | 0.35 | 1.81E−24 | A96 | G0s2 |
| Oat2 | 9.25E−29 | 0.4904 | 0.653 | 0.478 | 2.30E−24 | A96 | Oat |
| F5 | 5.35E−28 | 0.25658 | 0.858 | 0.754 | 1.33E−23 | A96 | F5 |
| Cox8a3 | 1.48E−27 | 0.25185 | 0.737 | 0.626 | 3.67E−23 | A96 | Cox8a |
| Gm49521 | 2.35E−27 | 0.25808 | 0.663 | 0.536 | 5.82E−23 | A96 | Gm4952 |
| Pxmp4 | 1.07E−26 | 0.25548 | 0.687 | 0.556 | 2.65E−22 | A96 | Pxmp4 |
| Ppp1r3b1 | 1.27E−26 | 0.25097 | 0.536 | 0.377 | 3.16E−22 | A96 | Ppp1r3b |
| Fmo1 | 3.23E−26 | 0.26495 | 0.606 | 0.446 | 8.01E−22 | A96 | Fmo1 |
| Zfp911 | 9.57E−26 | 0.27754 | 0.693 | 0.56 | 2.38E−21 | A96 | Zfp91 |
| Apol7a1 | 3.42E−23 | 0.25176 | 0.559 | 0.42 | 8.50E−19 | A96 | Apo17a |
| Ces1e | 5.02E−22 | 0.2566 | 0.71 | 0.608 | 1.25E−17 | A96 | Ces1e |
| Gadd45b | 5.16E−21 | 0.25977 | 0.452 | 0.311 | 1.28E−16 | A96 | Gadd45b |
| Etnppl3 | 6.58E−13 | 0.31543 | 0.677 | 0.619 | 1.63E−08 | A96 | Etnppl |
| Mup174 | 0 | 2.01662 | 1 | 0.947 | 0 | UT | Mup17 |
| Mup94 | 0 | 1.91449 | 0.976 | 0.594 | 0 | UT | Mup9 |
| Mup64 | 0 | 1.88184 | 0.992 | 0.804 | 0 | UT | Mup6 |
| Mup194 | 0 | 1.78587 | 1 | 0.916 | 0 | UT | Mup19 |
| Mup115 | 0 | 1.72843 | 1 | 0.84 | 0 | UT | Mup11 |
| Mup184 | 0 | 1.63857 | 0.997 | 0.838 | 0 | UT | Mup18 |
| Mup54 | 0 | 1.34199 | 0.839 | 0.541 | 0 | UT | Mup5 |
| Mup154 | 0 | 1.25956 | 0.617 | 0.166 | 0 | UT | Mup15 |
| Mup164 | 0 | 1.125 | 0.934 | 0.594 | 0 | UT | Mup16 |
| mt-Nd44 | 0 | 1.11568 | 0.998 | 0.942 | 0 | UT | mt-Nd4 |
| mt-Co24 | 0 | 1.07773 | 0.629 | 0.157 | 0 | UT | mt-Co2 |
| Gm137754 | 0 | 1.06127 | 0.761 | 0.306 | 0 | UT | Gm13775 |
| mt-Co14 | 0 | 0.9219 | 0.999 | 0.977 | 0 | UT | mt-Co1 |
| mt-Cytb3 | 0 | 0.87755 | 0.999 | 0.971 | 0 | UT | mt-Cytb |
| Mup214 | 0 | 0.87235 | 0.929 | 0.702 | 0 | UT | Mup21 |
| mt-Nd23 | 0 | 0.83252 | 0.999 | 0.978 | 0 | UT | mt-Nd2 |
| Selenbp23 | 0 | 0.83043 | 0.937 | 0.751 | 0 | UT | Selenbp2 |
| mt-Nd54 | 0 | 0.77968 | 0.995 | 0.942 | 0 | UT | mt-Nd5 |
| mt-Nd13 | 0 | 0.75999 | 1 | 0.978 | 0 | UT | mt-Nd1 |
| mt-Rnr22 | 0 | 0.54583 | 1 | 1 | 0 | UT | mt-Rnr2 |
| Mup32 | 0 | 0.54248 | 1 | 0.999 | 0 | UT | Mup3 |
| mt-Co34 | 1.54E−290 | 0.9584 | 0.603 | 0.198 | 3.82E−286 | UT | mt-Co3 |
| Serpina1e1 | 1.35E−287 | 0.46812 | 0.999 | 0.99 | 3.36E−283 | UT | Serpina1e |
| Akr1c62 | 4.68E−230 | 0.50466 | 0.971 | 0.92 | 1.16E−225 | UT | Akr1c6 |
| Ankrd554 | 6.63E−225 | 1.23086 | 0.696 | 0.451 | 1.65E−220 | UT | Ankrd55 |
| Mup43 | 8.62E−211 | 1.00077 | 0.898 | 0.716 | 2.14E−206 | UT | Mup4 |
| mt-Rnr13 | 1.37E−205 | 0.35383 | 1 | 1 | 3.40E−201 | UT | mt-Rnr1 |
| Fabp11 | 3.44E−187 | 0.30776 | 1 | 0.992 | 8.54E−183 | UT | Fabp1 |
| Elovl33 | 5.11E−177 | 0.52214 | 0.827 | 0.65 | 1.27E−172 | UT | Elovl3 |
| Gsta32 | 4.62E−168 | 0.40491 | 0.956 | 0.958 | 1.15E−163 | UT | Gsta3 |
| Mup74 | 2.96E−167 | 0.60153 | 0.998 | 0.937 | 7.35E−163 | UT | Mup7 |
| Nudt74 | 3.51E−163 | 0.61924 | 0.83 | 0.691 | 8.71E−159 | UT | Nudt7 |
| Serpina1a1 | 1.94E−157 | 0.33474 | 0.991 | 0.96 | 4.81E−153 | UT | Serpina1a |
| Uqcrq2 | 7.40E−157 | 0.41014 | 0.951 | 0.902 | 1.84E−152 | UT | Uqcrq |
| Car32 | 2.61E−147 | 0.37625 | 0.974 | 0.956 | 6.47E−143 | UT | Car3 |
| Ces3a2 | 1.36E−137 | 0.31735 | 0.967 | 0.93 | 3.38E−133 | UT | Ces3a |
| Mup84 | 1.52E−136 | 0.72676 | 0.373 | 0.13 | 3.77E−132 | UT | Mup8 |
| Adh11 | 1.12E−132 | 0.31221 | 0.96 | 0.941 | 2.79E−128 | UT | Adh1 |
| Prdx12 | 2.41E−131 | 0.34986 | 0.927 | 0.878 | 5.99E−127 | UT | Prdx1 |
| Gstp12 | 4.61E−130 | 0.60881 | 0.822 | 0.702 | 1.14E−125 | UT | Gstp1 |
| Hsd3b72 | 3.94E−128 | 0.45013 | 0.798 | 0.693 | 9.79E−124 | UT | Hsd3b7 |
| Acaa1b3 | 1.79E−119 | 0.3158 | 0.969 | 0.883 | 4.44E−115 | UT | Acaa1b |
| Mup201 | 2.74E−117 | 0.3725 | 1 | 0.992 | 6.81E−113 | UT | Mup20 |
| Mup133 | 3.18E−117 | 0.62497 | 0.546 | 0.351 | 7.90E−113 | UT | Mup13 |
| Rps142 | 1.62E−111 | 0.33642 | 0.917 | 0.856 | 4.03E−107 | UT | Rsp14 |
| Cyb52 | 5.75E−109 | 0.28445 | 0.983 | 0.972 | 1.43E−104 | UT | Cyb5 |
| 2810007J24Rik1 | 3.82E−107 | 0.28709 | 0.943 | 0.909 | 9.49E−103 | UT | 2810007J24Rik |
| Hsd3b54 | 2.16E−104 | 0.47119 | 0.587 | 0.367 | 5.37E−100 | UT | Hsd3b5 |
| Mup124 | 3.61E−104 | 0.37133 | 0.86 | 0.665 | 8.97E−100 | UT | Mup12 |
| Cox4i12 | 2.61E−94 | 0.26275 | 0.958 | 0.941 | 6.48E−90 | UT | Cox4i1 |
| Oaz11 | 5.51E−93 | 0.39443 | 0.75 | 0.651 | 1.37E−88 | UT | Oaz1 |
| Tmem2052 | 1.10E−91 | 0.29526 | 0.934 | 0.877 | 2.74E−87 | UT | Tmem205 |
| Serpina3c3 | 4.51E−84 | 0.42526 | 0.385 | 0.208 | 1.12E−79 | UT | Serpina3c |
| Ugt2b11 | 1.86E−83 | 0.30063 | 0.871 | 0.817 | 4.62E−79 | UT | Ugt2b1 |
| Rps23 | 3.51E−80 | 0.37937 | 0.757 | 0.637 | 8.73E−76 | UT | Rps2 |
| Inmt2 | 1.03E−77 | 0.36623 | 0.884 | 0.871 | 2.55E−73 | UT | Inmt |
| Cox6c2 | 1.98E−73 | 0.34318 | 0.884 | 0.808 | 4.92E−69 | UT | Cox6c |
| Ndufb112 | 7.98E−73 | 0.35219 | 0.735 | 0.637 | 1.98E−68 | UT | Ndufb11 |
| Cyp7b13 | 1.18E−72 | 0.35125 | 0.757 | 0.654 | 2.92E−68 | UT | Cyp7b1 |
| Cyp1a22 | 2.21E−72 | 0.30346 | 0.794 | 0.621 | 5.49E−68 | UT | Cyp1a2 |
| C8g2 | 9.19E−72 | 0.25825 | 0.91 | 0.859 | 2.28E−67 | UT | C8g |
| Lifr3 | 5.26E−70 | 0.35319 | 0.694 | 0.608 | 1.31E−65 | UT | Lifr |
| Hamp3 | 1.12E−69 | 0.27418 | 0.884 | 0.718 | 2.79E−65 | UT | Hamp |
| Cfhr21 | 1.47E−66 | 0.32911 | 0.868 | 0.854 | 3.64E−62 | UT | Cfhr2 |
| Mup143 | 2.61E−61 | 0.52442 | 0.616 | 0.469 | 6.49E−57 | UT | Mup14 |
| Aadac2 | 2.88E−59 | 0.32428 | 0.86 | 0.849 | 7.15E−55 | UT | Aadac |
| Keg13 | 4.07E−58 | 0.38569 | 0.545 | 0.441 | 1.01E−53 | UT | Keg1 |
| Cyp2a52 | 7.24E−58 | 0.32734 | 0.555 | 0.388 | 1.80E−53 | UT | Cyp2a5 |
| Chchd22 | 3.96E−57 | 0.2575 | 0.916 | 0.874 | 9.83E−53 | UT | Chchd2 |
| Ces3b1 | 1.20E−56 | 0.28399 | 0.787 | 0.727 | 2.98E−52 | UT | Ces3b |
| Ttc39c2 | 4.98E−56 | 0.31869 | 0.642 | 0.573 | 1.24E−51 | UT | Ttc39c |
| Gstm32 | 2.30E−53 | 0.47361 | 0.414 | 0.269 | 5.70E−49 | UT | Gstm3 |
| Gadd45g1 | 9.92E−53 | 0.34052 | 0.639 | 0.503 | 2.46E−48 | UT | Gadd45g |
| Ndufa42 | 7.60E−52 | 0.26172 | 0.889 | 0.851 | 1.89E−47 | UT | Ndufa4 |
| Cml11 | 7.90E−52 | 0.29532 | 0.757 | 0.709 | 1.96E−47 | UT | Cml1 |
| Thrsp3 | 3.45E−50 | 0.46025 | 0.627 | 0.583 | 8.56E−46 | UT | Thrsp |
| mt-Atp62 | 2.06E−48 | 0.41938 | 0.452 | 0.311 | 5.11E−44 | UT | mt-Atp6 |
| Rpl321 | 1.00E−47 | 0.30384 | 0.757 | 0.688 | 2.49E−43 | UT | Rpl32 |
| Hspe11 | 2.75E−44 | 0.30852 | 0.557 | 0.462 | 6.83E−40 | UT | Hspe1 |
| Hsd3b31 | 2.62E−41 | 0.28285 | 0.605 | 0.519 | 6.51E−37 | UT | Hsd3b3 |
| Serpina122 | 6.83E−41 | 0.27902 | 0.492 | 0.362 | 1.70E−36 | UT | Serpina12 |
| Prdx41 | 1.19E−38 | 0.2905 | 0.607 | 0.55 | 2.95E−34 | UT | Prdx4 |
| Mup24 | 3.60E−37 | 0.4469 | 0.392 | 0.285 | 8.95E−33 | UT | Mup2 |
| Cox7a22 | 2.72E−36 | 0.2511 | 0.784 | 0.722 | 6.76E−32 | UT | Cox7a2 |
| Cyp2b102 | 2.41E−35 | 0.50278 | 0.542 | 0.493 | 5.98E−31 | UT | Cyp2b10 |
| Ndufs62 | 3.36E−32 | 0.27205 | 0.724 | 0.678 | 8.33E−28 | UT | Ndufs6 |
| Ndufb41 | 1.61E−29 | 0.25309 | 0.49 | 0.409 | 3.99E−25 | UT | Ndufb4 |
| Rpl36al1 | 8.10E−27 | 0.27021 | 0.551 | 0.495 | 2.01E−22 | UT | Rpl36al |
| Mup116 | 1.57E−24 | 0.47001 | 0.583 | 0.522 | 3.91E−20 | UT | Mup1 |
| Rps152 | 4.98E−19 | 0.27219 | 0.528 | 0.49 | 1.24E−14 | UT | Rps15 |
| Ubb | 5.31E−19 | 0.26415 | 0.45 | 0.394 | 1.32E−14 | UT | Ubb |
| Ftl1 | 2.27E−15 | 0.30999 | 0.409 | 0.367 | 5.64E−11 | UT | Ftl1 |

Clear pericentral to periportal gradients were observed across all clusters except 6 hours post-APAP, due to the pericentral-specific injury in this model. Interestingly, a return of pericentral, Cyp2e1-expressing hepatocytes was observed by 24 hrs, which is earlier than peak regeneration time of 36 to 48 hours, suggesting possible gene expression reprogramming within existing surviving hepatocytes. Tables 2-7 provide differentially expressed genes between particular time points and untreated cells, time points A6, A24, A48A96, PHX3 FC.1, PHX3 FC.2 and PHX3 FC.15, respectively.

TABLE 2
Differentially expressed genes between time point A6 and untreated (UT).

| ID | p_val | avg_logFC | pct.1 | pct.2 | p_val_adj |
|---|---|---|---|---|---|
| Mt1 | 0 | 2.387707773 | 0.981 | 0.556 | 0 |
| Mt2 | 0 | 2.06273693 | 0.938 | 0.365 | 0 |
| Gclc | 0 | 1.902837282 | 0.962 | 0.718 | 0 |
| Srxn1 | 0 | 1.878352012 | 0.821 | 0.189 | 0 |
| Plin2 | 0 | 1.461295758 | 0.943 | 0.714 | 0 |
| Fabp1 | 0 | −1.016174848 | 0.971 | 1 | 0 |
| Akr1c6 | 0 | −1.586259995 | 0.675 | 0.971 | 0 |
| Mup17 | 0 | −2.354358557 | 0.871 | 1 | 0 |
| Mup19 | 0 | −2.396345291 | 0.759 | 1 | 0 |
| Mup11 | 0 | −3.213953203 | 0.511 | 1 | 0 |
| Gpx1 | 4.15457073465707e−311 | −0.768556331 | 0.969 | 0.999 | 1.03E−306 |
| Txnrd1 | 1.45669736974939e−310 | 1.443578942 | 0.838 | 0.441 | 3.62E−306 |
| Adh1 | 0.00E+00 | −1.255028206 | 0.74 | 0.96 | 2.34E−304 |
| Apoc1 | 1.03E−305 | −0.871282735 | 0.962 | 0.999 | 2.55E−301 |
| Mgst1 | 6.50E−299 | −1.060154854 | 0.938 | 0.997 | 1.61E−294 |
| Gm26924 | 3.46E−287 | 1.16085221 | 1 | 1 | 8.60E−283 |
| Krt18 | 5.85E−282 | 1.257881489 | 0.864 | 0.617 | 1.45E−277 |
| Actb | 9.88E−280 | 1.059835055 | 0.979 | 0.886 | 2.45E−275 |
| Mup3 | 9.44E−277 | −0.703483173 | 1 | 1 | 2.34E−272 |
| Krt8 | 3.88E−263 | 1.195874806 | 0.826 | 0.578 | 9.64E−259 |
| Cyp2e1 | 1.17E−251 | −2.318570161 | 0.162 | 0.911 | 2.90E−247 |
| Apoc4 | 1.91E−232 | −1.002265767 | 0.831 | 0.98 | 4.73E−228 |
| Mup16 | 1.87E−230 | −1.602085539 | 0.334 | 0.934 | 4.64E−226 |
| Serpina3m | 5.23E−230 | 0.967441963 | 0.85 | 0.722 | 1.30E−225 |
| Mup9 | 9.88E−226 | −2.004163996 | 0.489 | 0.976 | 2.45E−221 |
| Hmox1 | 2.37E−215 | 1.347585217 | 0.558 | 0.016 | 5.89E−211 |
| Tnfrsf12a | 5.66E−215 | 1.117506296 | 0.623 | 0.052 | 1.40E−210 |
| Apoc3 | 3.15E−214 | −0.768781074 | 0.869 | 0.993 | 7.83E−210 |
| Apoa2 | 1.12E−201 | −0.542784959 | 0.993 | 1 | 2.78E−197 |
| Apoa4 | 2.86E−200 | 1.367998503 | 0.721 | 0.339 | 7.09E−196 |
| Gm15564 | 3.29E−198 | 1.443066465 | 0.983 | 0.956 | 8.17E−194 |
| Rgn | 8.29E−196 | −1.573093006 | 0.243 | 0.874 | 2.06E−191 |
| Hbb-bs | 2.32E−195 | 2.862044849 | 0.537 | 0.018 | 5.76E−191 |
| Mup18 | 4.75E−195 | −1.627956781 | 0.728 | 0.997 | 1.18E−190 |
| Lars2 | 1.29E−190 | 1.391744455 | 1 | 0.994 | 3.20E−186 |
| Chka | 1.19E−189 | 1.375010255 | 0.716 | 0.231 | 2.97E−185 |
| Cdkn1a | 2.01E−187 | 1.2152557 | 0.702 | 0.195 | 4.98E−183 |
| Il1r1 | 4.05E−186 | 1.244999819 | 0.683 | 0.167 | 1.01E−181 |
| Car3 | 1.31E−184 | −1.036093133 | 0.854 | 0.974 | 3.26E−180 |
| Myh9 | 2.05E−183 | 1.160233135 | 0.718 | 0.318 | 5.08E−179 |
| Igfbp1 | 1.50E−181 | 1.058689301 | 0.792 | 0.717 | 3.71E−177 |
| Nudt7 | 5.65E−175 | −1.564266936 | 0.279 | 0.83 | 1.40E−170 |
| Slco1b2 | 3.15E−172 | −1.087378176 | 0.678 | 0.951 | 7.83E−168 |
| Mat1a | 5.22E−167 | 0.790765268 | 0.986 | 0.966 | 1.30E−162 |
| Hnf4a | 6.47E−166 | 0.879108718 | 0.835 | 0.743 | 1.61E−161 |
| Mup6 | 1.14E−164 | −1.871852037 | 0.706 | 0.992 | 2.82E−160 |
| Cyp4a14 | 1.96E−161 | 1.122498795 | 0.776 | 0.431 | 4.86E−157 |
| Fga | 3.87E−160 | 0.69335023 | 0.986 | 0.997 | 9.60E−156 |
| Slc39a14 | 4.45E−159 | 1.012749203 | 0.678 | 0.343 | 1.10E−154 |
| S100a9 | 1.09E−157 | 1.342849617 | 0.399 | 0.004 | 2.70E−153 |
| Lrrc58 | 6.48E−157 | 1.001096085 | 0.711 | 0.359 | 1.61E−152 |
| Cyp2c29 | 1.48E−156 | −1.149629979 | 0.418 | 0.93 | 3.68E−152 |
| Mup12 | 8.02E−156 | −1.269973728 | 0.32 | 0.86 | 1.99E−151 |
| Ppl | 2.58E−155 | 0.879840886 | 0.47 | 0.022 | 6.41E−151 |
| 2810007J24Rik | 4.24E−152 | −0.914301122 | 0.675 | 0.943 | 1.05E−147 |
| 1100001G20Rik | 3.93E−151 | −0.851607217 | 0.862 | 0.982 | 9.75E−147 |
| Selenbp2 | 5.10E−151 | −1.009165718 | 0.609 | 0.937 | 1.27E−146 |
| Serpina3n | 1.42E−146 | 0.848313239 | 0.747 | 0.574 | 3.52E−142 |
| Ttr | 1.21E−145 | −0.530625643 | 0.993 | 1 | 3.01E−141 |
| S100a8 | 4.44E−145 | 1.127879209 | 0.353 | 0.001 | 1.10E−140 |
| Slc16a6 | 3.23E−141 | 0.911357108 | 0.47 | 0.054 | 8.03E−137 |
| Mrap | 8.89E−139 | 0.884099926 | 0.606 | 0.272 | 2.21E−134 |
| Fgb | 1.34E−136 | 0.646934246 | 0.995 | 1 | 3.33E−132 |
| Uqcrq | 1.27E−135 | −0.88665513 | 0.721 | 0.951 | 3.15E−131 |
| Pck1 | 1.82E−134 | −0.652190949 | 0.814 | 0.987 | 4.52E−130 |
| Fam134b | 1.16E−133 | 0.893562894 | 0.678 | 0.405 | 2.87E−129 |
| Rnase4 | 3.04E−133 | −1.049196753 | 0.563 | 0.921 | 7.54E−129 |
| mt-Nd4 | 3.12E−131 | −0.638848134 | 0.964 | 0.998 | 7.74E−127 |
| Dbi | 6.97E−127 | −0.716365271 | 0.819 | 0.971 | 1.73E−122 |
| Fgg | 2.32E−125 | 0.615329068 | 0.986 | 0.991 | 5.76E−121 |
| Hilpda | 4.59E−125 | 0.778625914 | 0.403 | 0.024 | 1.14E−120 |
| Hsd3b7 | 1.37E−123 | −1.0059958 | 0.387 | 0.798 | 3.41E−119 |
| Cyb5 | 2.11E−122 | −0.71018958 | 0.916 | 0.983 | 5.23E−118 |
| Hsd3b5 | 1.42E−120 | −1.069520224 | 0.062 | 0.587 | 3.51E−116 |
| Gsta3 | 1.52E−120 | −0.806290466 | 0.885 | 0.956 | 3.76E−116 |
| Oat | 2.95E−120 | −1.224853019 | 0.05 | 0.611 | 7.33E−116 |
| Trp53inp1 | 3.61E−120 | 1.008414088 | 0.637 | 0.308 | 8.96E−116 |
| Cyp2a5 | 1.79E−119 | −1.498910166 | 0.019 | 0.555 | 4.44E−115 |
| Hamp | 6.15E−119 | −1.02112368 | 0.422 | 0.884 | 1.53E−114 |
| Apoe | 1.31E−118 | −0.368966531 | 1 | 1 | 3.25E−114 |
| Psen2 | 1.68E−118 | −0.947373772 | 0.384 | 0.774 | 4.17E−114 |
| Tmem205 | 3.80E−116 | −0.774742732 | 0.695 | 0.934 | 9.43E−112 |
| Mup15 | 1.54E−114 | −1.316464146 | 0.076 | 0.617 | 3.83E−110 |
| Cyp3a11 | 2.00E−114 | −0.423130745 | 0.964 | 1 | 4.96E−110 |
| Sqstm1 | 1.89E−113 | 0.743150456 | 0.859 | 0.805 | 4.70E−109 |
| Acaa1b | 2.10E−113 | −0.732823459 | 0.752 | 0.969 | 5.21E−109 |
| Mup7 | 6.91E−111 | −0.941370494 | 0.831 | 0.998 | 1.72E−106 |
| Cyp8b1 | 2.65E−108 | −0.889229825 | 0.282 | 0.745 | 6.59E−104 |
| Gm13775 | 5.84E−107 | −1.065618856 | 0.284 | 0.761 | 1.45E−102 |
| Bcl2l1 | 5.29E−106 | 0.775118224 | 0.501 | 0.122 | 1.31E−101 |
| Insig2 | 9.46E−104 | 0.669991504 | 0.854 | 0.81 | 2.35E−99 |
| Tubb2a | 1.34E−101 | 0.749433744 | 0.618 | 0.431 | 3.32E−97 |
| Sdc4 | 1.77E−100 | 0.619597869 | 0.909 | 0.867 | 4.40E−96 |
| Iigp1 | 2.96E−100 | 0.768753667 | 0.78 | 0.759 | 7.35E−96 |
| Klf6 | 1.89E−98 | 0.732407935 | 0.382 | 0.034 | 4.69E−94 |
| Eif1a | 2.08E−98 | 0.795281298 | 0.599 | 0.289 | 5.16E−94 |
| Rps27l | 3.09E−97 | −0.865927484 | 0.484 | 0.849 | 7.67E−93 |
| Mup21 | 4.96E−97 | −0.827249928 | 0.58 | 0.929 | 1.23E−92 |
| Cyp2d9 | 5.06E−97 | −0.640698605 | 0.747 | 0.959 | 1.26E−92 |
| Ccl6 | 7.03E−97 | 0.78914235 | 0.274 | 0.006 | 1.75E−92 |
| Ugt2b5 | 7.03E−97 | −0.603351248 | 0.847 | 0.959 | 1.75E−92 |
| Adck3 | 1.08E−96 | −0.87611538 | 0.363 | 0.764 | 2.67E−92 |
| Tubb6 | 2.37E−96 | 0.576687679 | 0.368 | 0.03 | 5.89E−92 |
| G6pc | 4.32E−96 | −0.960058274 | 0.313 | 0.752 | 1.07E−91 |
| Cltc | 7.08E−96 | 0.784985399 | 0.661 | 0.414 | 1.76E−91 |
| Hbb-bt | 2.95E−95 | 1.430134393 | 0.246 | 0.002 | 7.33E−91 |
| Srgn | 2.10E−94 | 0.934067453 | 0.284 | 0.01 | 5.22E−90 |
| Sec1412 | 2.77E−94 | −0.789737513 | 0.484 | 0.856 | 6.88E−90 |
| Cyp1a2 | 1.21E−93 | −0.993935836 | 0.353 | 0.794 | 3.00E−89 |
| mt-Nd1 | 2.49E−93 | −0.388336084 | 0.983 | 1 | 6.19E−89 |
| Epas1 | 2.59E−93 | 0.775763153 | 0.604 | 0.297 | 6.43E−89 |
| Ets2 | 3.90E−93 | 0.695623802 | 0.415 | 0.056 | 9.69E−89 |
| Ttc36 | 8.91E−93 | −0.660645659 | 0.768 | 0.95 | 2.21E−88 |
| Ugt2b1 | 2.20E−92 | −0.776816931 | 0.561 | 0.871 | 5.46E−88 |
| Cox6c | 1.10E−91 | −0.844280539 | 0.558 | 0.884 | 2.73E−87 |
| Btg2 | 1.58E−91 | 0.832086378 | 0.549 | 0.241 | 3.93E−87 |
| Xbp1 | 5.43E−91 | 0.603165814 | 0.692 | 0.57 | 1.35E−86 |
| Hspb8 | 7.33E−90 | 0.633933124 | 0.709 | 0.597 | 1.82E−85 |
| Sod1 | 8.95E−90 | −0.439319795 | 0.931 | 0.983 | 2.22E−85 |
| Cers6 | 1.45E−89 | 0.532818648 | 0.332 | 0.022 | 3.61E−85 |
| 2010003K11Rik | 8.77E−89 | 0.588354735 | 0.396 | 0.046 | 2.18E−84 |
| Hgd | 9.82E−89 | −0.758572265 | 0.511 | 0.861 | 2.44E−84 |
| Urah | 5.16E−88 | −0.764109132 | 0.525 | 0.852 | 1.28E−83 |
| H3f3b | 2.47E−86 | 0.702914634 | 0.613 | 0.419 | 6.14E−82 |
| Picalm | 2.64E−86 | 0.754529703 | 0.647 | 0.376 | 6.55E−82 |
| Bach1 | 1.42E−85 | 0.832333098 | 0.461 | 0.151 | 3.52E−81 |
| Eif5 | 1.50E−85 | 0.576301866 | 0.816 | 0.74 | 3.73E−81 |
| Ugdh | 2.38E−84 | 0.633076584 | 0.721 | 0.604 | 5.92E−80 |
| Aldh3a2 | 2.55E−84 | −0.834229334 | 0.064 | 0.529 | 6.32E−80 |
| Ddx3x | 2.89E−84 | 0.656890035 | 0.692 | 0.513 | 7.19E−80 |
| Gjb2 | 4.51E−84 | 0.60822545 | 0.678 | 0.572 | 1.12E−79 |
| Rcan1 | 1.03E−82 | 0.707283787 | 0.425 | 0.085 | 2.56E−78 |
| Nid1 | 1.05E−82 | 0.342599117 | 0.229 | 0.003 | 2.60E−78 |
| Hsd3b3 | 1.20E−82 | −0.779501123 | 0.174 | 0.605 | 2.98E−78 |
| Keg1 | 2.46E−82 | −0.824548137 | 0.134 | 0.545 | 6.12E−78 |
| Cxadr | 3.65E−82 | 0.698450944 | 0.606 | 0.345 | 9.07E−78 |
| Hsd17b13 | 3.30E−80 | 0.680828119 | 0.864 | 0.74 | 8.20E−76 |
| Il1b | 3.56E−80 | 0.742893545 | 0.236 | 0.005 | 8.84E−76 |
| Ubc | 8.91E−80 | −0.661839716 | 0.573 | 0.872 | 2.21E−75 |
| Stat3 | 1.03E−79 | 0.715400235 | 0.604 | 0.318 | 2.55E−75 |
| Asl | 1.49E−79 | 0.558638879 | 0.826 | 0.787 | 3.70E−75 |
| Ces3a | 4.62E−79 | −0.52533635 | 0.831 | 0.967 | 1.15E−74 |
| Psat1 | 4.90E−79 | 0.301248562 | 0.229 | 0.004 | 1.22E−74 |
| C8g | 5.15E−79 | −0.654510262 | 0.673 | 0.91 | 1.28E−74 |
| Gulo | 5.44E−79 | −0.801665727 | 0.076 | 0.497 | 1.35E−74 |
| Itih3 | 2.22E−78 | 0.546240791 | 0.907 | 0.89 | 5.51E−74 |
| Abca1 | 4.43E−78 | 0.724477701 | 0.604 | 0.377 | 1.10E−73 |
| Pon1 | 8.86E−78 | −0.685751275 | 0.685 | 0.906 | 2.20E−73 |
| Scp2 | 2.00E−77 | −0.519124916 | 0.866 | 0.986 | 4.98E−73 |
| Clec4f | 1.18E−76 | 1.108180782 | 0.339 | 0.043 | 2.94E−72 |
| Eif4g2 | 6.68E−76 | 0.559692663 | 0.757 | 0.678 | 1.66E−71 |
| Oaf | 1.37E−75 | −0.7328674 | 0.437 | 0.794 | 3.40E−71 |
| Slc22a1 | 8.96E−75 | −0.797941816 | 0.36 | 0.747 | 2.23E−70 |
| n-R5-8s1 | 1.47E−74 | 0.766100662 | 0.308 | 0.031 | 3.65E−70 |
| Rell1 | 1.19E−73 | 0.591181865 | 0.375 | 0.063 | 2.97E−69 |
| Hpd | 1.20E−73 | −0.518180386 | 0.883 | 0.976 | 2.98E−69 |
| Gpt2 | 1.39E−73 | 0.539114072 | 0.764 | 0.725 | 3.45E−69 |
| Sat1 | 1.97E−73 | 0.68869896 | 0.47 | 0.167 | 4.89E−69 |
| Ddx21 | 2.38E−73 | 0.73138069 | 0.535 | 0.22 | 5.90E−69 |
| Mfsd2a | 6.35E−73 | 0.666142747 | 0.465 | 0.148 | 1.58E−68 |
| Cox4i1 | 2.09E−72 | −0.515989169 | 0.869 | 0.958 | 5.19E−68 |
| Pcbd1 | 2.64E−72 | −0.687761883 | 0.535 | 0.837 | 6.57E−68 |
| Mup5 | 4.12E−72 | −1.23728834 | 0.475 | 0.839 | 1.02E−67 |
| Cyp2c37 | 5.90E−72 | −0.886903784 | 0.177 | 0.608 | 1.47E−67 |
| Eef2 | 5.96E−72 | 0.525855561 | 0.9 | 0.873 | 1.48E−67 |
| Ugt2b36 | 6.92E−72 | −0.583431487 | 0.745 | 0.93 | 1.72E−67 |
| Mbl1 | 1.03E−71 | −0.713006005 | 0.382 | 0.749 | 2.56E−67 |
| Ctsl | 1.19E−71 | 0.530101805 | 0.878 | 0.857 | 2.95E−67 |
| mt-Co2 | 1.33E−71 | −1.105332856 | 0.2 | 0.629 | 3.31E−67 |
| Hacl1 | 1.55E−71 | −0.706897734 | 0.489 | 0.797 | 3.86E−67 |
| D10Wsu102e | 1.62E−71 | 0.58780186 | 0.37 | 0.063 | 4.02E−67 |
| Ankrd55 | 1.94E−71 | −1.250834631 | 0.372 | 0.696 | 4.83E−67 |
| Nop58 | 2.01E−71 | 0.663449653 | 0.492 | 0.162 | 4.99E−67 |
| Hint1 | 2.70E−71 | −0.673880575 | 0.57 | 0.858 | 6.70E−67 |
| Retsat | 4.73E−71 | 0.491096059 | 0.845 | 0.83 | 1.18E−66 |
| Hagh | 4.00E−70 | −0.674009711 | 0.573 | 0.858 | 9.93E−66 |
| Rora | 6.90E−70 | 0.644354953 | 0.549 | 0.341 | 1.71E−65 |
| Ndufa4 | 1.71E−69 | −0.683126695 | 0.656 | 0.889 | 4.24E−65 |
| Upp2 | 2.20E−67 | −0.69025963 | 0.274 | 0.673 | 5.46E−63 |
| Lpin2 | 3.08E−67 | 0.686687426 | 0.745 | 0.597 | 7.64E−63 |
| Wbp1l | 1.00E−66 | 0.553507326 | 0.697 | 0.606 | 2.49E−62 |
| Tnfrsf1a | 2.12E−66 | 0.599573483 | 0.508 | 0.244 | 5.27E−62 |
| Itih2 | 6.50E−65 | 0.501091194 | 0.893 | 0.884 | 1.61E−60 |
| Inmt | 9.14E−65 | −0.731016404 | 0.709 | 0.884 | 2.27E−60 |
| Apob | 1.91E−64 | 0.580156235 | 0.99 | 0.989 | 4.73E−60 |
| Dusp5 | 2.61E−64 | 0.296374788 | 0.196 | 0.005 | 6.47E−60 |
| Gm24601 | 3.58E−64 | 0.46836359 | 0.241 | 0.015 | 8.89E−60 |
| Aldh8a1 | 3.87E−64 | −0.683676902 | 0.418 | 0.758 | 9.60E−60 |
| Lpgat1 | 4.83E−64 | 0.595219291 | 0.618 | 0.502 | 1.20E−59 |
| Hba-a1 | 4.96E−64 | 0.734957385 | 0.165 | 0.001 | 1.23E−59 |
| Glyat | 1.28E−63 | −0.647687911 | 0.468 | 0.798 | 3.18E−59 |
| Lect2 | 4.58E−63 | −0.626967771 | 0.045 | 0.414 | 1.14E−58 |
| Plscr1 | 4.87E−63 | 0.444489505 | 0.329 | 0.047 | 1.21E−58 |
| Cidec | 8.20E−63 | 0.299384206 | 0.253 | 0.019 | 2.04E−58 |
| Uqcr11 | 1.20E−62 | −0.603079991 | 0.606 | 0.879 | 2.97E−58 |
| Tmsb4x | 1.25E−62 | 0.780616209 | 0.332 | 0.057 | 3.10E−58 |
| Hebp1 | 2.37E−62 | −0.635432267 | 0.43 | 0.75 | 5.88E−58 |
| Hes6 | 2.98E−62 | −0.561302344 | 0.076 | 0.42 | 7.39E−58 |
| Atp5e | 5.29E−62 | −0.650247857 | 0.506 | 0.832 | 1.31E−57 |
| mmu-mir-6240 | 8.16E−62 | 1.101237189 | 0.413 | 0.115 | 2.03E−57 |
| Efhd2 | 2.27E−61 | 0.583831769 | 0.468 | 0.148 | 5.63E−57 |
| Msrb1 | 2.71E−61 | −0.59903268 | 0.558 | 0.838 | 6.72E−57 |
| 8430408G22Rik | 2.51E−60 | −0.50210224 | 0.041 | 0.392 | 6.22E−56 |
| Tlcd2 | 4.44E−60 | −0.594498821 | 0.186 | 0.536 | 1.10E−55 |
| Serpina1a | 5.95E−60 | −0.378457074 | 0.914 | 0.991 | 1.48E−55 |
| Leap2 | 1.65E−59 | −0.671300937 | 0.26 | 0.632 | 4.10E−55 |
| Rdh7 | 1.97E−59 | −0.418625957 | 0.862 | 0.969 | 4.88E−55 |
| Gcnt2 | 2.40E−59 | 0.537509676 | 0.372 | 0.082 | 5.96E−55 |
| Uox | 2.52E−59 | −0.553115511 | 0.905 | 0.965 | 6.26E−55 |
| Acss2 | 9.19E−59 | −0.620071374 | 0.248 | 0.574 | 2.28E−54 |
| Prdx1 | 1.04E−58 | −0.529229113 | 0.742 | 0.927 | 2.58E−54 |
| Lrg1 | 1.21E−58 | 0.490792829 | 0.783 | 0.736 | 2.99E−54 |
| Mup13 | 1.81E−58 | −0.77574306 | 0.205 | 0.546 | 4.49E−54 |
| Cox7b | 4.10E−58 | −0.642240776 | 0.632 | 0.878 | 1.02E−53 |
| Myo1e | 4.83E−58 | 0.614341001 | 0.475 | 0.182 | 1.20E−53 |
| Mmp8 | 7.38E−58 | 0.359005863 | 0.136 | 0 | 1.83E−53 |
| Ttc39c | 7.39E−58 | −0.636274928 | 0.325 | 0.642 | 1.84E−53 |
| Gn13 | 7.72E−58 | 0.575970422 | 0.501 | 0.204 | 1.92E−53 |
| B4galt5 | 1.31E−57 | 0.476306558 | 0.332 | 0.07 | 3.26E−53 |
| Mup4 | 1.88E−57 | −0.99530818 | 0.621 | 0.898 | 4.67E−53 |
| Ndufa6 | 2.60E−57 | −0.613649978 | 0.506 | 0.798 | 6.47E−53 |
| Ang | 4.44E−57 | −0.662883759 | 0.315 | 0.687 | 1.10E−52 |
| Alb | 8.54E−57 | 0.420298986 | 1 | 1 | 2.12E−52 |
| Cyp7b1 | 8.74E−57 | −0.65802273 | 0.42 | 0.757 | 2.17E−52 |
| C230081A13Rik | 8.95E−57 | 0.426976229 | 0.279 | 0.035 | 2.22E−52 |
| Fn1 | 1.95E−56 | 0.521230804 | 0.847 | 0.821 | 4.85E−52 |
| Cox7a2 | 2.11E−56 | −0.650338386 | 0.456 | 0.784 | 5.23E−52 |
| Man2a1 | 2.93E−56 | 0.497115325 | 0.671 | 0.583 | 7.27E−52 |
| Cyp2c50 | 3.04E−56 | −0.661823297 | 0.616 | 0.888 | 7.54E−52 |
| Jmjd1c | 4.80E−56 | 0.636619821 | 0.47 | 0.178 | 1.19E−51 |
| Apol7a | 6.85E−56 | −0.492294435 | 0.072 | 0.382 | 1.70E−51 |
| Jak1 | 6.86E−56 | 0.603850485 | 0.554 | 0.342 | 1.70E−51 |
| Haao | 7.05E−56 | −0.591181029 | 0.621 | 0.861 | 1.75E−51 |
| St3gal5 | 2.58E−55 | 0.607639775 | 0.706 | 0.49 | 6.40E−51 |
| Ctss | 5.17E−55 | 0.764167083 | 0.212 | 0.019 | 1.28E−50 |
| Ywhag | 1.12E−54 | 0.58241474 | 0.518 | 0.308 | 2.78E−50 |
| Rarres2 | 1.90E−54 | −0.552194576 | 0.678 | 0.905 | 4.73E−50 |
| Mthfd2 | 2.03E−54 | 0.262448025 | 0.205 | 0.012 | 5.03E−50 |
| St3gal1 | 3.04E−54 | 0.528739882 | 0.473 | 0.221 | 7.55E−50 |
| Fdx1 | 3.17E−54 | −0.623439092 | 0.305 | 0.625 | 7.87E−50 |
| Chchd2 | 3.70E−54 | −0.552503492 | 0.718 | 0.916 | 9.20E−50 |
| Lactb2 | 4.06E−54 | −0.596505052 | 0.47 | 0.751 | 1.01E−49 |
| Phlda1 | 4.10E−54 | 0.549174347 | 0.494 | 0.356 | 1.02E−49 |
| Cd14 | 1.17E−53 | 0.362089625 | 0.153 | 0.003 | 2.91E−49 |
| 2-Mar | 1.24E−53 | −0.55994977 | 0.611 | 0.865 | 3.08E−49 |
| Aass | 1.93E−53 | 0.477517138 | 0.747 | 0.716 | 4.80E−49 |
| Clqb | 2.33E−53 | 0.681679654 | 0.234 | 0.024 | 5.78E−49 |
| Hsd17b2 | 3.46E−53 | −0.595878526 | 0.391 | 0.711 | 8.60E−49 |
| Cml1 | 1.92E−52 | −0.61424052 | 0.475 | 0.757 | 4.76E−48 |
| Adh6-ps1 | 6.36E−52 | −0.418504072 | 0.017 | 0.313 | 1.58E−47 |
| Tyrobp | 6.73E−52 | 0.466551501 | 0.189 | 0.011 | 1.67E−47 |
| Retnlg | 8.59E−52 | 0.375148336 | 0.129 | 0 | 2.13E−47 |
| Arhgef12 | 1.06E−51 | 0.590764816 | 0.578 | 0.408 | 2.62E−47 |
| Pah | 1.69E−51 | −0.52207695 | 0.687 | 0.891 | 4.20E−47 |
| Rock2 | 3.56E−51 | 0.594094466 | 0.425 | 0.131 | 8.83E−47 |
| Tra2a | 3.81E−51 | 0.712328169 | 0.337 | 0.1 | 9.46E−47 |
| Creb3l2 | 4.81E−51 | 0.427180051 | 0.329 | 0.068 | 1.19E−46 |
| Etf1 | 5.70E−51 | 0.559444816 | 0.606 | 0.409 | 1.41E−46 |
| Ddx5 | 1.56E−50 | 0.522944475 | 0.685 | 0.515 | 3.88E−46 |
| Cd302 | 1.61E−50 | −0.549925078 | 0.649 | 0.86 | 3.99E−46 |
| Arl4d | 1.86E−50 | −0.507228317 | 0.112 | 0.437 | 4.61E−46 |
| Grn | 3.15E−50 | 0.540768685 | 0.582 | 0.396 | 7.83E−46 |
| Agpat9 | 3.32E−50 | 0.327040595 | 0.277 | 0.038 | 8.25E−46 |
| Adh4 | 3.58E−50 | −0.526868306 | 0.165 | 0.521 | 8.89E−46 |
| Lifr | 6.21E−50 | −0.626180925 | 0.389 | 0.694 | 1.54E−45 |
| Hba-a2 | 7.25E−50 | 0.553546734 | 0.131 | 0.001 | 1.80E−45 |
| Tiparp | 1.75E−49 | 0.545567123 | 0.346 | 0.087 | 4.34E−45 |
| Gamt | 2.68E−49 | −0.560282685 | 0.277 | 0.591 | 6.66E−45 |
| C3 | 5.98E−49 | 0.44276078 | 0.993 | 0.99 | 1.49E−44 |
| Fxyd5 | 7.03E−49 | 0.274279595 | 0.138 | 0.002 | 1.74E−44 |
| Cd5l | 9.18E−49 | 0.693485826 | 0.21 | 0.026 | 2.28E−44 |
| Fkbp5 | 1.09E−48 | 0.516128063 | 0.415 | 0.117 | 2.70E−44 |
| Gsta1 | 1.15E−48 | 0.407606856 | 0.32 | 0.062 | 2.85E−44 |
| Atp2a2 | 1.30E−48 | 0.494962728 | 0.616 | 0.495 | 3.22E−44 |
| Ppib | 1.44E−48 | −0.589699514 | 0.463 | 0.767 | 3.58E−44 |
| Ndufb2 | 3.05E−48 | −0.577154458 | 0.301 | 0.629 | 7.57E−44 |
| 1-Mar | 3.12E−48 | −0.477762617 | 0.644 | 0.885 | 7.75E−44 |
| Mcm10 | 3.33E−48 | −0.487427572 | 0.079 | 0.386 | 8.27E−44 |
| Dusp6 | 3.94E−48 | 0.59164596 | 0.482 | 0.261 | 9.78E−44 |
| Fabp2 | 5.11E−48 | −0.615569339 | 0.315 | 0.622 | 1.27E−43 |
| Stk40 | 8.11E−48 | 0.536418312 | 0.382 | 0.121 | 2.01E−43 |
| Ddt | 9.71E−48 | −0.522221147 | 0.539 | 0.829 | 2.41E−43 |
| Hif1a | 1.06E−47 | 0.516911277 | 0.427 | 0.166 | 2.63E−43 |
| Ppm1k | 1.93E−47 | −0.558394496 | 0.15 | 0.45 | 4.79E−43 |
| Akr1c20 | 2.15E−47 | −0.554152364 | 0.248 | 0.564 | 5.34E−43 |
| Csf1r | 2.32E−47 | 0.704458586 | 0.184 | 0.015 | 5.76E−43 |
| Aadac | 2.83E−47 | −0.619958028 | 0.673 | 0.86 | 7.04E−43 |
| Saa4 | 3.63E−47 | −0.571021905 | 0.437 | 0.749 | 9.02E−43 |
| Pemt | 5.32E−47 | −0.551184095 | 0.492 | 0.78 | 1.32E−42 |
| Atp5h | 5.87E−47 | −0.6398091 | 0.377 | 0.682 | 1.46E−42 |
| Slc25a47 | 7.64E−47 | 0.428932953 | 0.869 | 0.927 | 1.90E−42 |
| Ranbp2 | 8.46E−47 | 0.598499679 | 0.566 | 0.338 | 2.10E−42 |
| Aldh1a1 | 1.08E−46 | −0.478797447 | 0.862 | 0.956 | 2.68E−42 |
| Ptprc | 1.10E−46 | 0.416505491 | 0.153 | 0.006 | 2.72E−42 |
| Lyz2 | 2.06E−46 | 0.732183369 | 0.234 | 0.032 | 5.12E−42 |
| Abhd2 | 2.14E−46 | 0.484366625 | 0.37 | 0.105 | 5.33E−42 |
| 1700017B05Rik | 2.19E−46 | 0.330656885 | 0.255 | 0.037 | 5.43E−42 |
| Ndufb9 | 2.30E−46 | −0.503828812 | 0.661 | 0.888 | 5.71E−42 |
| Pla2g7 | 2.76E−46 | 0.282344956 | 0.119 | 0.001 | 6.85E−42 |
| Adamts1 | 3.06E−46 | 0.282556845 | 0.2 | 0.018 | 7.60E−42 |
| Malat1 | 3.47E−46 | 0.920628097 | 0.871 | 0.78 | 8.61E−42 |
| Cox6a1 | 3.93E−46 | −0.48722865 | 0.675 | 0.887 | 9.76E−42 |
| Gstz1 | 5.09E−46 | −0.464086834 | 0.73 | 0.905 | 1.26E−41 |
| 1600014C10Rik | 7.24E−46 | −0.498182953 | 0.167 | 0.462 | 1.80E−41 |
| Aff4 | 8.92E−46 | 0.616309327 | 0.537 | 0.337 | 2.21E−41 |
| Clec4d | 1.07E−45 | 0.26359638 | 0.107 | 0 | 2.66E−41 |
| Egr1 | 1.20E−45 | 0.584558888 | 0.589 | 0.447 | 2.98E−41 |
| Atp5j2 | 1.26E−45 | −0.51308051 | 0.606 | 0.852 | 3.12E−41 |
| Eaf1 | 1.85E−45 | 0.521796522 | 0.427 | 0.168 | 4.59E−41 |
| Qdpr | 2.35E−45 | −0.52357376 | 0.606 | 0.814 | 5.84E−41 |
| Il1r2 | 2.51E−45 | 0.323447978 | 0.117 | 0.001 | 6.22E−41 |
| Tars | 3.11E−45 | 0.492059498 | 0.666 | 0.539 | 7.73E−41 |
| Mcl1 | 5.48E−45 | 0.570934356 | 0.573 | 0.371 | 1.36E−40 |
| Serpina1d | 5.92E−45 | −0.289011223 | 0.926 | 0.988 | 1.47E−40 |
| Ncl | 1.05E−44 | 0.48450982 | 0.69 | 0.567 | 2.60E−40 |
| Gm4952 | 1.47E−44 | −0.49867829 | 0.198 | 0.533 | 3.64E−40 |
| Gchfr | 2.75E−44 | −0.554783473 | 0.489 | 0.768 | 6.83E−40 |
| Ndufa7 | 2.91E−44 | −0.555912274 | 0.406 | 0.718 | 7.23E−40 |
| Dio1 | 3.74E−44 | −0.545283744 | 0.253 | 0.542 | 9.29E−40 |
| Cyp7a1 | 4.21E−44 | −0.431201844 | 0.01 | 0.259 | 1.04E−39 |
| Mpc2 | 5.18E−44 | −0.482839417 | 0.599 | 0.857 | 1.29E−39 |
| Son | 6.25E−44 | 0.542911509 | 0.601 | 0.358 | 1.55E−39 |
| Thrsp | 7.87E−44 | −0.724961807 | 0.384 | 0.627 | 1.95E−39 |
| Nit2 | 9.38E−44 | −0.485940613 | 0.301 | 0.577 | 2.33E−39 |
| Btg1 | 1.09E−43 | 0.458007046 | 0.356 | 0.101 | 2.70E−39 |
| Epb4.1 | 1.81E−43 | 0.562789274 | 0.537 | 0.304 | 4.50E−39 |
| Spred1 | 2.85E−43 | 0.388422711 | 0.308 | 0.069 | 7.08E−39 |
| Tm4sf4 | 3.17E−43 | 0.512552747 | 0.484 | 0.256 | 7.87E−39 |
| Vcam1 | 4.97E−43 | 0.457401491 | 0.134 | 0.004 | 1.23E−38 |
| Atp5f1 | 6.07E−43 | −0.517061165 | 0.69 | 0.869 | 1.51E−38 |
| Cldn14 | 6.28E−43 | 0.364540783 | 0.32 | 0.076 | 1.56E−38 |
| Adh5 | 7.79E−43 | −0.491433382 | 0.649 | 0.854 | 1.94E−38 |
| Cyp2c70 | 8.22E−43 | −0.551134023 | 0.587 | 0.834 | 2.04E−38 |
| Gm26917 | 1.07E−42 | 0.60120474 | 0.382 | 0.181 | 2.66E−38 |
| Cd163 | 2.23E−42 | 0.440845529 | 0.146 | 0.008 | 5.54E−38 |
| Tmem87b | 3.21E−42 | 0.434399269 | 0.351 | 0.096 | 7.98E−38 |
| Csad | 4.42E−42 | −0.559786018 | 0.473 | 0.744 | 1.10E−37 |
| Elovl3 | 5.34E−42 | −0.506724135 | 0.568 | 0.827 | 1.33E−37 |
| Cyp4a10 | 6.21E−42 | 0.456645767 | 0.687 | 0.657 | 1.54E−37 |
| Mup8 | 8.20E−42 | −0.752577709 | 0.098 | 0.373 | 2.04E−37 |
| Ctsb | 8.90E−42 | 0.521310448 | 0.804 | 0.781 | 2.21E−37 |
| Iqgap1 | 9.52E−42 | 0.45544473 | 0.162 | 0.012 | 2.36E−37 |
| Herpud1 | 1.01E−41 | −0.497586072 | 0.535 | 0.803 | 2.51E−37 |
| Rplp1 | 1.47E−41 | −0.504662964 | 0.449 | 0.757 | 3.64E−37 |
| Ndufb6 | 1.70E−41 | −0.507682563 | 0.475 | 0.759 | 4.21E−37 |
| Khk | 2.56E−41 | −0.491453217 | 0.702 | 0.863 | 6.36E−37 |
| Heatr1 | 3.13E−41 | 0.406005371 | 0.291 | 0.062 | 7.78E−37 |
| Dhrs3 | 3.16E−41 | −0.467621234 | 0.198 | 0.465 | 7.85E−37 |
| Crp | 3.31E−41 | −0.506869364 | 0.477 | 0.756 | 8.23E−37 |
| Plek | 3.32E−41 | 0.308857869 | 0.11 | 0.001 | 8.24E−37 |
| Ebpl | 8.84E−41 | −0.450334658 | 0.196 | 0.451 | 2.20E−36 |
| Trf | 9.22E−41 | −0.261883505 | 0.988 | 1 | 2.29E−36 |
| Akr1c14 | 1.02E−40 | −0.518570798 | 0.413 | 0.707 | 2.54E−36 |
| Cox5b | 4.90E−40 | −0.498783616 | 0.425 | 0.726 | 1.22E−35 |
| Ndufb11 | 5.25E−40 | −0.509671057 | 0.461 | 0.735 | 1.30E−35 |
| Dusp16 | 5.69E−40 | 0.46051738 | 0.372 | 0.149 | 1.41E−35 |
| Rnf144a | 5.85E−40 | 0.313046134 | 0.177 | 0.015 | 1.45E−35 |
| Sult1a1 | 6.44E−40 | 0.318989978 | 0.685 | 0.707 | 1.60E−35 |
| Kctd3 | 6.81E−40 | 0.260833465 | 0.239 | 0.036 | 1.69E−35 |
| Serpina1e | 7.25E−40 | −0.268413792 | 0.99 | 0.999 | 1.80E−35 |
| Lcn2 | 1.07E−39 | 0.412194792 | 0.301 | 0.07 | 2.67E−35 |
| Wfdc17 | 1.33E−39 | 0.486653465 | 0.177 | 0.018 | 3.31E−35 |
| Hibadh | 2.75E−39 | −0.499409672 | 0.542 | 0.791 | 6.84E−35 |
| Rpl32 | 4.14E−39 | −0.532218378 | 0.501 | 0.757 | 1.03E−34 |
| Lyst | 4.87E−39 | 0.409280587 | 0.31 | 0.073 | 1.21E−34 |
| Errfi1 | 5.94E−39 | 0.393680047 | 0.943 | 0.911 | 1.47E−34 |
| Apoh | 6.38E−39 | −0.262314298 | 0.924 | 0.986 | 1.58E−34 |
| Tmem256 | 7.18E−39 | −0.518510202 | 0.375 | 0.667 | 1.78E−34 |
| Atp5j | 8.66E−39 | −0.48373528 | 0.492 | 0.757 | 2.15E−34 |
| Chic2 | 1.01E−38 | 0.419790279 | 0.37 | 0.128 | 2.51E−34 |
| Fxyd1 | 1.67E−38 | −0.479908398 | 0.432 | 0.721 | 4.15E−34 |
| Dot1l | 2.53E−38 | 0.397036294 | 0.282 | 0.066 | 6.29E−34 |
| Blvrb | 2.64E−38 | −0.545112721 | 0.286 | 0.564 | 6.56E−34 |
| Atp1a1 | 3.98E−38 | 0.419079339 | 0.53 | 0.457 | 9.89E−34 |
| Sord | 5.54E−38 | −0.383628337 | 0.754 | 0.931 | 1.38E−33 |
| Ssr4 | 6.38E−38 | −0.507577243 | 0.413 | 0.687 | 1.58E−33 |
| Selenbp1 | 6.88E−38 | −0.493707332 | 0.401 | 0.699 | 1.71E−33 |
| Tbc1d15 | 9.36E−38 | 0.488646353 | 0.461 | 0.244 | 2.32E−33 |
| Ndufs6 | 1.21E−37 | −0.537929827 | 0.482 | 0.724 | 3.01E−33 |
| Fndc3b | 1.22E−37 | 0.409645989 | 0.315 | 0.096 | 3.04E−33 |
| C1qc | 1.37E−37 | 0.586092716 | 0.172 | 0.018 | 3.40E−33 |
| Tsr1 | 1.46E−37 | 0.360287403 | 0.341 | 0.097 | 3.63E−33 |
| Slc10a2 | 1.62E−37 | 0.299006165 | 0.255 | 0.05 | 4.03E−33 |
| Gnmt | 2.11E−37 | −0.33340449 | 0.962 | 0.988 | 5.24E−33 |
| Lonp2 | 2.72E−37 | −0.467365882 | 0.561 | 0.814 | 6.76E−33 |
| Pabpc1 | 3.30E−37 | 0.410051805 | 0.697 | 0.592 | 8.20E−33 |
| Rsp14 | 3.48E−37 | −0.402093042 | 0.745 | 0.917 | 8.64E−33 |
| Chchd10 | 3.74E−37 | −0.40191686 | 0.802 | 0.944 | 9.28E−33 |
| C1qa | 4.78E−37 | 0.514846404 | 0.174 | 0.02 | 1.19E−32 |
| Dgkd | 5.13E−37 | 0.370844881 | 0.282 | 0.069 | 1.27E−32 |
| Ppp1r3b | 7.31E−37 | −0.402657788 | 0.098 | 0.38 | 1.82E−32 |
| Atp5k | 1.05E−36 | −0.47725773 | 0.368 | 0.678 | 2.61E−32 |
| Fam107b | 1.11E−36 | 0.463320341 | 0.449 | 0.238 | 2.75E−32 |
| Rarres1 | 1.54E−36 | −0.455632451 | 0.155 | 0.42 | 3.84E−32 |
| Cd74 | 1.65E−36 | 0.453937562 | 0.155 | 0.012 | 4.09E−32 |
| Cald1 | 1.80E−36 | 0.373703655 | 0.766 | 0.749 | 4.47E−32 |
| Cyp2c67 | 1.95E−36 | −0.463488581 | 0.126 | 0.418 | 4.85E−32 |
| Klf15 | 2.28E−36 | −0.451831953 | 0.208 | 0.473 | 5.65E−32 |
| Kif5b | 2.63E−36 | 0.50116686 | 0.494 | 0.303 | 6.52E−32 |
| Reep3 | 6.36E−36 | 0.409014614 | 0.535 | 0.422 | 1.58E−31 |
| Rgs2 | 8.14E−36 | 0.395729601 | 0.138 | 0.01 | 2.02E−31 |
| Rad54l2 | 1.08E−35 | 0.388705156 | 0.325 | 0.092 | 2.69E−31 |
| Ndufb4 | 1.39E−35 | −0.453693039 | 0.212 | 0.49 | 3.46E−31 |
| Hic2 | 1.39E−35 | 0.289767391 | 0.15 | 0.012 | 3.46E−31 |
| Laptm5 | 1.89E−35 | 0.338180771 | 0.124 | 0.006 | 4.69E−31 |
| Tjp2 | 2.38E−35 | 0.411461753 | 0.332 | 0.114 | 5.91E−31 |
| Ndufa13 | 2.55E−35 | −0.43876131 | 0.549 | 0.802 | 6.33E−31 |
| Tmed5 | 2.73E−35 | 0.401633325 | 0.594 | 0.492 | 6.77E−31 |
| Lgmn | 2.88E−35 | 0.484586871 | 0.246 | 0.059 | 7.14E−31 |
| Mettl7b | 2.95E−35 | −0.452656656 | 0.647 | 0.869 | 7.34E−31 |
| Ptpn1 | 3.04E−35 | 0.41633121 | 0.353 | 0.107 | 7.55E−31 |
| Cd44 | 4.20E−35 | 0.303745221 | 0.115 | 0.004 | 1.04E−30 |
| H2-Ab1 | 4.30E−35 | 0.460956687 | 0.158 | 0.015 | 1.07E−30 |
| Cd97 | 4.52E−35 | 0.345774315 | 0.115 | 0.004 | 1.12E−30 |
| 1500017E21Rik | 5.38E−35 | −0.460997215 | 0.217 | 0.476 | 1.34E−30 |
| Cyp3a25 | 6.56E−35 | −0.386094858 | 0.728 | 0.919 | 1.63E−30 |
| Nolc1 | 8.57E−35 | 0.47222269 | 0.363 | 0.134 | 2.13E−30 |
| Hpgd | 9.04E−35 | −0.387752401 | 0.115 | 0.394 | 2.25E−30 |
| Nars | 1.16E−34 | 0.437769953 | 0.606 | 0.471 | 2.88E−30 |
| Dst | 1.47E−34 | 0.510971252 | 0.43 | 0.206 | 3.66E−30 |
| Mdn1 | 1.76E−34 | 0.317989246 | 0.251 | 0.051 | 4.37E−30 |
| Mbnl1 | 2.34E−34 | 0.451258107 | 0.492 | 0.297 | 5.81E−30 |
| Comt | 2.52E−34 | −0.368377448 | 0.776 | 0.931 | 6.25E−30 |
| Agmat | 2.99E−34 | −0.471832829 | 0.339 | 0.604 | 7.44E−30 |
| Stard10 | 3.47E−34 | −0.338316568 | 0.876 | 0.967 | 8.61E−30 |
| Mat2a | 4.43E−34 | 0.482570924 | 0.487 | 0.279 | 1.10E−29 |
| Plec | 4.56E−34 | 0.467512335 | 0.422 | 0.186 | 1.13E−29 |
| Mrp114 | 5.17E−34 | −0.459040925 | 0.258 | 0.523 | 1.28E−29 |
| Gm24187 | 5.89E−34 | 0.431940663 | 0.2 | 0.032 | 1.46E−29 |
| Hspe1 | 6.06E−34 | −0.480734946 | 0.294 | 0.557 | 1.50E−29 |
| Gtpbp4 | 6.17E−34 | 0.482239467 | 0.477 | 0.262 | 1.53E−29 |
| Etfb | 1.03E−33 | −0.365490277 | 0.802 | 0.94 | 2.57E−29 |
| Tsku | 1.17E−33 | 0.36762763 | 0.327 | 0.095 | 2.90E−29 |
| Brd2 | 1.82E−33 | 0.571515166 | 0.542 | 0.408 | 4.52E−29 |
| Tacc2 | 2.29E−33 | 0.329332932 | 0.289 | 0.071 | 5.70E−29 |
| Uqcr10 | 2.44E−33 | −0.45190185 | 0.561 | 0.8 | 6.05E−29 |
| Ell2 | 2.61E−33 | 0.417241283 | 0.535 | 0.416 | 6.49E−29 |
| Hpx | 3.10E−33 | −0.303288229 | 0.988 | 0.997 | 7.71E−29 |
| H6pd | 3.17E−33 | 0.343718187 | 0.623 | 0.595 | 7.88E−29 |
| Tsc22d2 | 3.40E−33 | 0.391059638 | 0.353 | 0.124 | 8.44E−29 |
| Rpl36al | 3.73E−33 | −0.475541391 | 0.284 | 0.551 | 9.26E−29 |
| Sdad1 | 4.01E−33 | 0.312421192 | 0.289 | 0.072 | 9.96E−29 |
| Itgb2 | 6.11E−33 | 0.272302012 | 0.105 | 0.004 | 1.52E−28 |
| Cyp4a12a | 6.66E−33 | −0.456827492 | 0.317 | 0.597 | 1.65E−28 |
| Coa6 | 7.34E−33 | −0.411409685 | 0.15 | 0.373 | 1.82E−28 |
| Sc5d | 8.33E−33 | −0.500900917 | 0.58 | 0.776 | 2.07E−28 |
| Cd53 | 8.60E−33 | 0.265579528 | 0.103 | 0.003 | 2.13E−28 |
| mt-Co3 | 1.07E−32 | −0.705930608 | 0.346 | 0.603 | 2.65E−28 |
| Cml2 | 1.08E−32 | −0.397916138 | 0.647 | 0.857 | 2.68E−28 |
| Gabarap | 1.15E−32 | −0.442546824 | 0.518 | 0.769 | 2.85E−28 |
| Tns1 | 1.37E−32 | 0.434939232 | 0.403 | 0.196 | 3.40E−28 |
| Fitm1 | 2.00E−32 | −0.324153911 | 0.012 | 0.208 | 4.96E−28 |
| B2m | 2.22E−32 | −0.330354919 | 0.871 | 0.963 | 5.52E−28 |
| Chchd1 | 2.83E−32 | −0.439819068 | 0.21 | 0.438 | 7.03E−28 |
| Fcgr3 | 3.13E−32 | 0.308247077 | 0.107 | 0.004 | 7.76E−28 |
| Tcerg1 | 3.42E−32 | 0.357549618 | 0.305 | 0.093 | 8.50E−28 |
| C9 | 3.52E−32 | −0.395507776 | 0.828 | 0.938 | 8.73E−28 |
| Map2k3 | 4.21E−32 | 0.434654367 | 0.427 | 0.208 | 1.04E−27 |
| Kdelr2 | 4.26E−32 | 0.397729248 | 0.484 | 0.355 | 1.06E−27 |
| Litaf | 5.05E−32 | 0.394366845 | 0.451 | 0.278 | 1.25E−27 |
| Slc20a1 | 6.40E−32 | 0.357248475 | 0.284 | 0.076 | 1.59E−27 |
| Mup14 | 8.41E−32 | −0.697469805 | 0.358 | 0.616 | 2.09E−27 |
| Maff | 8.86E−32 | 0.355300257 | 0.224 | 0.048 | 2.20E−27 |
| Tfpi2 | 9.96E−32 | −0.366342462 | 0.155 | 0.39 | 2.47E−27 |
| Aldob | 1.09E−31 | 0.315339576 | 0.998 | 0.996 | 2.69E−27 |
| Eif4g1 | 1.61E−31 | 0.374860037 | 0.723 | 0.669 | 4.01E−27 |
| Cfp | 1.68E−31 | 0.356178244 | 0.143 | 0.015 | 4.17E−27 |
| Pbld1 | 1.90E−31 | −0.442542986 | 0.315 | 0.565 | 4.73E−27 |
| Dcxr | 1.94E−31 | −0.440851615 | 0.37 | 0.631 | 4.82E−27 |
| Minos1 | 2.22E−31 | −0.455548025 | 0.332 | 0.595 | 5.51E−27 |
| Mug2 | 2.25E−31 | 0.368295871 | 0.776 | 0.738 | 5.60E−27 |
| 1110001J03Rik | 2.82E−31 | −0.432785224 | 0.215 | 0.456 | 7.01E−27 |
| Tatdn2 | 2.96E−31 | 0.373296072 | 0.334 | 0.111 | 7.35E−27 |
| Cmbl | 4.73E−31 | −0.442706435 | 0.415 | 0.678 | 1.18E−26 |
| Ifitm3 | 4.74E−31 | −0.425091425 | 0.587 | 0.812 | 1.18E−26 |
| Adipor2 | 6.85E−31 | −0.473791512 | 0.578 | 0.807 | 1.70E−26 |
| Upb1 | 9.41E−31 | −0.418695518 | 0.499 | 0.759 | 2.34E−26 |
| Amacr | 1.21E−30 | −0.413582585 | 0.294 | 0.549 | 3.00E−26 |
| Nr2f6 | 1.57E−30 | −0.375433952 | 0.234 | 0.434 | 3.90E−26 |
| Bcl3 | 1.68E−30 | 0.289017645 | 0.277 | 0.074 | 4.16E−26 |
| Pigr | 2.46E−30 | 0.348711695 | 0.947 | 0.947 | 6.10E−26 |
| Chac2 | 3.07E−30 | −0.286511682 | 0.057 | 0.251 | 7.62E−26 |
| Uqcrh | 3.79E−30 | −0.429164519 | 0.589 | 0.81 | 9.41E−26 |
| Lrrfip1 | 4.30E−30 | 0.445928709 | 0.37 | 0.156 | 1.07E−25 |
| Ccs | 8.22E−30 | −0.426694207 | 0.42 | 0.676 | 2.04E−25 |
| Atox1 | 9.01E−30 | −0.446203219 | 0.382 | 0.633 | 2.24E−25 |
| Wdr26 | 9.80E−30 | 0.432510772 | 0.549 | 0.406 | 2.43E−25 |
| Mrp63 | 1.18E−29 | −0.442201567 | 0.277 | 0.531 | 2.93E−25 |
| Rnd3 | 1.20E−29 | 0.36548808 | 0.284 | 0.091 | 2.98E−25 |
| Neat1 | 1.49E−29 | 0.63740381 | 0.31 | 0.135 | 3.71E−25 |
| Bbox1 | 1.94E−29 | −0.408245616 | 0.308 | 0.548 | 4.83E−25 |
| Cyp39a1 | 2.10E−29 | 0.351128984 | 0.305 | 0.11 | 5.21E−25 |
| Timm13 | 2.45E−29 | −0.432961802 | 0.418 | 0.671 | 6.08E−25 |
| Gclm | 2.74E−29 | 0.269864161 | 0.695 | 0.712 | 6.80E−25 |
| Sdc3 | 2.77E−29 | 0.359804895 | 0.103 | 0.005 | 6.89E−25 |
| Acsl1 | 3.83E−29 | −0.343424014 | 0.752 | 0.924 | 9.51E−25 |
| Hint2 | 4.70E−29 | −0.424321102 | 0.425 | 0.676 | 1.17E−24 |
| 1500011K16Rik | 4.73E−29 | −0.329639846 | 0.095 | 0.312 | 1.18E−24 |
| Slc38a3 | 4.87E−29 | −0.40978981 | 0.663 | 0.858 | 1.21E−24 |
| Asgr1 | 4.98E−29 | −0.385655364 | 0.656 | 0.851 | 1.24E−24 |
| Vkorc1 | 5.02E−29 | −0.404176916 | 0.296 | 0.54 | 1.25E−24 |
| Cyp2j5 | 5.47E−29 | −0.398305774 | 0.687 | 0.879 | 1.36E−24 |
| H2-Eb1 | 5.57E−29 | 0.327872672 | 0.105 | 0.006 | 1.38E−24 |
| Cyb5b | 6.61E−29 | −0.423345561 | 0.482 | 0.724 | 1.64E−24 |
| 2410015M20Rik | 7.01E−29 | −0.402123463 | 0.344 | 0.604 | 1.74E−24 |
| Apobec1 | 7.36E−29 | 0.305082858 | 0.239 | 0.058 | 1.83E−24 |
| Myl12a | 8.33E−29 | 0.390780681 | 0.461 | 0.281 | 2.07E−24 |
| Mybbp1a | 8.99E−29 | 0.431161557 | 0.396 | 0.19 | 2.23E−24 |
| Vsig4 | 9.72E−29 | 0.483290635 | 0.117 | 0.013 | 2.41E−24 |
| H2-D1 | 1.04E−28 | 0.286522764 | 0.766 | 0.774 | 2.58E−24 |
| Pfkfb2 | 1.32E−28 | 0.290016291 | 0.26 | 0.067 | 3.27E−24 |
| Atp13a3 | 1.35E−28 | 0.394480252 | 0.532 | 0.41 | 3.36E−24 |
| Commd6 | 1.55E−28 | −0.364529559 | 0.172 | 0.39 | 3.85E−24 |
| Serpina12 | 2.42E−28 | −0.453399356 | 0.232 | 0.492 | 6.01E−24 |
| Cfhr2 | 2.70E−28 | −0.44846316 | 0.692 | 0.868 | 6.70E−24 |
| Mup1 | 2.73E−28 | −0.737727332 | 0.344 | 0.583 | 6.77E−24 |
| Alad | 3.06E−28 | −0.420641438 | 0.286 | 0.513 | 7.60E−24 |
| Glo1 | 3.46E−28 | −0.406574019 | 0.513 | 0.749 | 8.60E−24 |
| Psme1 | 4.27E−28 | −0.394006165 | 0.26 | 0.5 | 1.06E−23 |
| Zbtb20 | 4.83E−28 | 0.471532978 | 0.566 | 0.445 | 1.20E−23 |
| Fpgs | 5.94E−28 | −0.34940945 | 0.146 | 0.348 | 1.48E−23 |
| Ppan | 6.61E−28 | 0.272410087 | 0.255 | 0.067 | 1.64E−23 |
| Rpl13a | 6.99E−28 | −0.381462137 | 0.704 | 0.868 | 1.74E−23 |
| Tmem56 | 7.12E−28 | 0.273557142 | 0.582 | 0.612 | 1.77E−23 |
| Mpeg1 | 7.48E−28 | 0.493478112 | 0.263 | 0.089 | 1.86E−23 |
| Gnl2 | 7.97E−28 | 0.390473036 | 0.384 | 0.171 | 1.98E−23 |
| Rps18 | 8.83E−28 | −0.488540797 | 0.337 | 0.578 | 2.19E−23 |
| Gnas | 9.32E−28 | 0.258853087 | 0.79 | 0.82 | 2.31E−23 |
| Serbp1 | 1.38E−27 | 0.322652522 | 0.659 | 0.634 | 3.42E−23 |
| Cyp2c54 | 1.66E−27 | −0.39493764 | 0.499 | 0.759 | 4.12E−23 |
| Hnrnpu | 2.07E−27 | 0.339415607 | 0.625 | 0.582 | 5.14E−23 |
| Bphl | 2.18E−27 | −0.40747923 | 0.339 | 0.579 | 5.40E−23 |
| Cp | 2.19E−27 | 0.254076812 | 0.89 | 0.943 | 5.44E−23 |
| Etnppl | 2.75E−27 | 0.41816318 | 0.706 | 0.606 | 6.83E−23 |
| Nme1 | 3.88E−27 | −0.432885306 | 0.425 | 0.622 | 9.63E−23 |
| Nr1i3 | 3.91E−27 | −0.412654588 | 0.165 | 0.377 | 9.71E−23 |
| Slc4a4 | 5.25E−27 | 0.420259653 | 0.513 | 0.336 | 1.30E−22 |
| 0610005C13Rik | 5.61E−27 | −0.425135941 | 0.427 | 0.653 | 1.39E−22 |
| Sds | 5.65E−27 | 0.390741627 | 0.73 | 0.67 | 1.40E−22 |
| Steap4 | 6.28E−27 | 0.437231109 | 0.37 | 0.222 | 1.56E−22 |
| Mrps24 | 6.38E−27 | −0.390380395 | 0.31 | 0.566 | 1.58E−22 |
| Chd1 | 6.53E−27 | 0.378763061 | 0.377 | 0.166 | 1.62E−22 |
| App | 6.61E−27 | 0.361832458 | 0.315 | 0.114 | 1.64E−22 |
| Ndufa3 | 6.89E−27 | −0.41160475 | 0.344 | 0.598 | 1.71E−22 |
| Hmgcr | 6.96E−27 | 0.416711628 | 0.425 | 0.272 | 1.73E−22 |
| Sptbn1 | 7.29E−27 | 0.42268289 | 0.439 | 0.26 | 1.81E−22 |
| Acy3 | 7.41E−27 | −0.313748988 | 0.131 | 0.332 | 1.84E−22 |
| Tmem14c | 8.44E−27 | −0.368514287 | 0.332 | 0.585 | 2.10E−22 |
| Arg1 | 8.49E−27 | 0.317948822 | 0.983 | 0.982 | 2.11E−22 |
| Prrc2c | 9.31E−27 | 0.439271845 | 0.415 | 0.201 | 2.31E−22 |
| Hsp90ab1 | 9.80E−27 | 0.322748941 | 0.916 | 0.922 | 2.43E−22 |
| Saa2 | 1.07E−26 | 0.558658405 | 0.315 | 0.222 | 2.66E−22 |
| Usmg5 | 1.10E−26 | −0.43049483 | 0.2 | 0.43 | 2.73E−22 |
| Cyp2c68 | 1.26E−26 | −0.398830868 | 0.298 | 0.558 | 3.13E−22 |
| Gfra1 | 1.28E−26 | 0.386382804 | 0.599 | 0.498 | 3.18E−22 |
| Por | 1.45E−26 | 0.335963246 | 0.704 | 0.675 | 3.61E−22 |
| Nop56 | 1.55E−26 | 0.265670844 | 0.282 | 0.084 | 3.85E−22 |
| Hdac11 | 1.65E−26 | −0.33684651 | 0.1 | 0.285 | 4.10E−22 |
| Sparcl1 | 1.73E−26 | −0.253616986 | 0 | 0.136 | 4.30E−22 |
| Ndufa2 | 1.97E−26 | −0.396473631 | 0.492 | 0.738 | 4.89E−22 |
| Mmp14 | 2.07E−26 | 0.288913135 | 0.253 | 0.073 | 5.15E−22 |
| Spp2 | 2.20E−26 | −0.382096801 | 0.334 | 0.582 | 5.47E−22 |
| Pecr | 2.77E−26 | −0.398084036 | 0.446 | 0.68 | 6.88E−22 |
| Lmo4 | 2.79E−26 | 0.320534245 | 0.308 | 0.116 | 6.94E−22 |
| Kmo | 3.86E−26 | −0.384440188 | 0.487 | 0.732 | 9.59E−22 |
| Rps15 | 4.78E−26 | −0.455155041 | 0.301 | 0.528 | 1.19E−21 |
| Uhrf1bp1l | 5.09E−26 | 0.419802385 | 0.399 | 0.207 | 1.26E−21 |
| Igfbp2 | 5.12E−26 | −0.298981027 | 0.68 | 0.885 | 1.27E−21 |
| Acsm1 | 6.31E−26 | −0.369245009 | 0.527 | 0.756 | 1.57E−21 |
| Slc22a30 | 6.77E−26 | −0.374598404 | 0.282 | 0.493 | 1.68E−21 |
| Ddx6 | 6.90E−26 | 0.444275069 | 0.527 | 0.366 | 1.71E−21 |
| Elovl5 | 7.23E−26 | 0.335236661 | 0.63 | 0.58 | 1.79E−21 |
| Lrrc59 | 7.61E−26 | 0.377173076 | 0.422 | 0.28 | 1.89E−21 |
| Gm23388 | 8.75E−26 | 0.416208382 | 0.222 | 0.057 | 2.17E−21 |
| Ces3b | 9.42E−26 | −0.36637965 | 0.57 | 0.787 | 2.34E−21 |
| Ccbl1 | 1.10E−25 | −0.36488302 | 0.205 | 0.423 | 2.72E−21 |
| Serinc3 | 1.16E−25 | 0.35928911 | 0.525 | 0.433 | 2.87E−21 |
| Hdlbp | 1.27E−25 | 0.345721467 | 0.737 | 0.713 | 3.14E−21 |
| Akap13 | 1.55E−25 | 0.495253767 | 0.406 | 0.218 | 3.85E−21 |
| Dkc1 | 1.58E−25 | 0.32575964 | 0.315 | 0.112 | 3.93E−21 |
| Prdx4 | 1.64E−25 | −0.417588892 | 0.372 | 0.607 | 4.08E−21 |
| Slc35d1 | 2.13E−25 | 0.309435259 | 0.489 | 0.435 | 5.29E−21 |
| Shmt1 | 2.34E−25 | −0.393237106 | 0.339 | 0.556 | 5.80E−21 |
| Hnrnpdl | 2.40E−25 | 0.397994117 | 0.406 | 0.214 | 5.97E−21 |
| Rps2 | 2.97E−25 | −0.387862655 | 0.525 | 0.757 | 7.37E−21 |
| Gne | 2.99E−25 | −0.425142761 | 0.263 | 0.469 | 7.41E−21 |
| Ier5 | 3.24E−25 | 0.288994609 | 0.134 | 0.019 | 8.04E−21 |
| Cebpa | 3.27E−25 | −0.39155301 | 0.442 | 0.672 | 8.13E−21 |
| Gsk3b | 3.56E−25 | 0.406458835 | 0.384 | 0.193 | 8.85E−21 |
| Ddc | 3.65E−25 | −0.360752783 | 0.241 | 0.46 | 9.07E−21 |
| Nr0b2 | 3.70E−25 | −0.293010319 | 0.086 | 0.28 | 9.19E−21 |
| Mettl20 | 3.72E−25 | −0.331367185 | 0.119 | 0.327 | 9.23E−21 |
| Ndufc2 | 4.83E−25 | −0.425284817 | 0.36 | 0.579 | 1.20E−20 |
| Fmo5 | 5.02E−25 | 0.286219883 | 0.823 | 0.837 | 1.25E−20 |
| Uqcrb | 6.09E−25 | −0.408416744 | 0.425 | 0.641 | 1.51E−20 |
| Slc40a1 | 6.95E−25 | 0.466969696 | 0.456 | 0.328 | 1.73E−20 |
| Pdia6 | 7.01E−25 | −0.389966782 | 0.47 | 0.712 | 1.74E−20 |
| Tmem243 | 7.37E−25 | −0.379053275 | 0.327 | 0.557 | 1.83E−20 |
| Abhd14b | 9.11E−25 | −0.39366782 | 0.327 | 0.565 | 2.26E−20 |
| D10Jhu81e | 9.68E−25 | −0.395730917 | 0.334 | 0.564 | 2.40E−20 |
| Stom | 9.70E−25 | 0.323229029 | 0.329 | 0.133 | 2.41E−20 |
| Cox8a | 1.39E−24 | −0.399332537 | 0.396 | 0.621 | 3.45E−20 |
| Gadd45g | 1.42E−24 | −0.355407686 | 0.375 | 0.639 | 3.53E−20 |
| Wdr43 | 1.64E−24 | 0.343830267 | 0.353 | 0.146 | 4.08E−20 |
| Timm8b | 1.92E−24 | −0.393581904 | 0.291 | 0.525 | 4.76E−20 |
| Baz1a | 2.07E−24 | 0.259610685 | 0.26 | 0.077 | 5.13E−20 |
| Sfpq | 2.16E−24 | 0.393977901 | 0.406 | 0.211 | 5.37E−20 |
| Ucp2 | 2.17E−24 | 0.261164666 | 0.107 | 0.01 | 5.40E−20 |
| Cisd1 | 2.20E−24 | −0.372694857 | 0.451 | 0.686 | 5.47E−20 |
| Mak16 | 2.52E−24 | 0.317126116 | 0.267 | 0.087 | 6.26E−20 |
| Chuk | 2.65E−24 | 0.372437977 | 0.523 | 0.421 | 6.57E−20 |
| Cyp27a1 | 2.85E−24 | −0.373510332 | 0.64 | 0.837 | 7.08E−20 |
| Lcp1 | 3.45E−24 | 0.484011001 | 0.492 | 0.303 | 8.57E−20 |
| Ivns1abp | 3.73E−24 | 0.356610399 | 0.446 | 0.315 | 9.26E−20 |
| Ptpn12 | 3.88E−24 | 0.33417878 | 0.263 | 0.084 | 9.63E−20 |
| Tsc22d1 | 4.00E−24 | −0.299728636 | 0.064 | 0.242 | 9.94E−20 |
| Eef1a1 | 4.24E−24 | 0.282586942 | 0.835 | 0.85 | 1.05E−19 |
| 1110008F13Rik | 5.38E−24 | −0.37509166 | 0.411 | 0.648 | 1.34E−19 |
| Cox6b1 | 6.04E−24 | −0.362017449 | 0.594 | 0.809 | 1.50E−19 |
| Akr1a1 | 6.54E−24 | −0.326935362 | 0.671 | 0.857 | 1.62E−19 |
| Gsr | 6.57E−24 | 0.412829579 | 0.453 | 0.303 | 1.63E−19 |
| Msn | 8.02E−24 | 0.260172491 | 0.105 | 0.009 | 1.99E−19 |
| Atp5g3 | 8.63E−24 | −0.300221819 | 0.764 | 0.911 | 2.14E−19 |
| Pxmp2 | 9.00E−24 | −0.367129885 | 0.325 | 0.55 | 2.24E−19 |
| Ifrd1 | 9.29E−24 | 0.490389826 | 0.365 | 0.185 | 2.31E−19 |
| Atp5c1 | 1.13E−23 | −0.347648538 | 0.587 | 0.792 | 2.82E−19 |
| Cldn3 | 1.20E−23 | −0.314469359 | 0.143 | 0.355 | 2.98E−19 |
| Gstm4 | 1.23E−23 | −0.380861133 | 0.234 | 0.444 | 3.06E−19 |
| Gjb1 | 1.36E−23 | −0.31656535 | 0.788 | 0.916 | 3.38E−19 |
| Lap3 | 1.43E−23 | −0.364371999 | 0.573 | 0.782 | 3.55E−19 |
| Tpr | 1.48E−23 | 0.440921219 | 0.501 | 0.339 | 3.67E−19 |
| Smg1 | 1.87E−23 | 0.334990596 | 0.265 | 0.106 | 4.65E−19 |
| Anp32a | 1.90E−23 | −0.316957742 | 0.193 | 0.413 | 4.71E−19 |
| Purb | 1.96E−23 | 0.35248294 | 0.341 | 0.158 | 4.88E−19 |
| Dnaja2 | 1.97E−23 | 0.372342331 | 0.516 | 0.402 | 4.88E−19 |
| AA474408 | 1.98E−23 | 0.347079511 | 0.274 | 0.094 | 4.90E−19 |
| 15-Sep | 1.98E−23 | −0.409167133 | 0.487 | 0.689 | 4.93E−19 |
| Phc2 | 1.99E−23 | 0.31203626 | 0.284 | 0.111 | 4.93E−19 |
| Apom | 2.12E−23 | −0.302410414 | 0.663 | 0.862 | 5.27E−19 |
| Mrps18c | 2.20E−23 | −0.366291099 | 0.181 | 0.394 | 5.47E−19 |
| Fcna | 2.82E−23 | 0.377428055 | 0.124 | 0.02 | 7.01E−19 |
| Ghr | 2.91E−23 | −0.364775879 | 0.647 | 0.833 | 7.23E−19 |
| Mien1 | 3.12E−23 | −0.370437762 | 0.27 | 0.489 | 7.74E−19 |
| Palld | 3.45E−23 | 0.341357023 | 0.353 | 0.167 | 8.57E−19 |
| Cars | 3.86E−23 | 0.315814572 | 0.329 | 0.135 | 9.57E−19 |
| Hsd17b10 | 3.95E−23 | −0.377206484 | 0.477 | 0.7 | 9.81E−19 |
| Cyp3a13 | 4.37E−23 | 0.284411943 | 0.613 | 0.569 | 1.08E−18 |
| Gm2788 | 4.67E−23 | −0.283138796 | 0.072 | 0.264 | 1.16E−18 |
| Cast | 4.88E−23 | 0.400886525 | 0.399 | 0.213 | 1.21E−18 |
| Mocs2 | 5.04E−23 | −0.346681141 | 0.372 | 0.604 | 1.25E−18 |
| Sirt3 | 5.14E−23 | −0.345997558 | 0.203 | 0.413 | 1.28E−18 |
| Hist1h1c | 6.02E−23 | −0.300906532 | 0.143 | 0.372 | 1.49E−18 |
| Eprs | 6.03E−23 | 0.37008595 | 0.566 | 0.488 | 1.50E−18 |
| Tgoln1 | 6.08E−23 | 0.265747623 | 0.601 | 0.575 | 1.51E−18 |
| Metap2 | 6.57E−23 | 0.368604828 | 0.453 | 0.285 | 1.63E−18 |
| Hsp90b1 | 6.73E−23 | −0.261479921 | 0.811 | 0.953 | 1.67E−18 |
| Tmem258 | 6.87E−23 | −0.27689533 | 0.115 | 0.282 | 1.70E−18 |
| Adtrp | 7.01E−23 | −0.366413277 | 0.332 | 0.547 | 1.74E−18 |
| Cideb | 7.62E−23 | −0.368365389 | 0.442 | 0.676 | 1.89E−18 |
| Scd1 | 8.10E−23 | −0.466125013 | 0.871 | 0.901 | 2.01E−18 |
| Dgat2 | 8.46E−23 | −0.343266251 | 0.558 | 0.767 | 2.10E−18 |
| S100a1 | 9.78E−23 | −0.374338118 | 0.453 | 0.661 | 2.43E−18 |
| Echs1 | 1.11E−22 | −0.331606352 | 0.642 | 0.817 | 2.76E−18 |
| C8b | 1.12E−22 | −0.315917765 | 0.702 | 0.881 | 2.78E−18 |
| Tgm2 | 1.18E−22 | 0.399330712 | 0.446 | 0.258 | 2.93E−18 |
| BC005537 | 1.44E−22 | 0.354337911 | 0.544 | 0.45 | 3.57E−18 |
| Zfp683 | 1.44E−22 | −0.421689112 | 0 | 0.116 | 3.58E−18 |
| Gyk | 1.72E−22 | 0.368418571 | 0.465 | 0.35 | 4.28E−18 |
| Cnpy2 | 1.77E−22 | −0.372850093 | 0.365 | 0.566 | 4.41E−18 |
| Ndufc1 | 1.95E−22 | −0.344432714 | 0.291 | 0.515 | 4.83E−18 |
| Cpt1a | 1.98E−22 | 0.261119591 | 0.654 | 0.651 | 4.93E−18 |
| Ethe1 | 2.35E−22 | −0.333417645 | 0.286 | 0.49 | 5.84E−18 |
| Bola3 | 2.38E−22 | −0.368862428 | 0.313 | 0.525 | 5.91E−18 |
| Tceb2 | 2.43E−22 | −0.32073626 | 0.203 | 0.398 | 6.04E−18 |
| 3110043021Rik | 2.85E−22 | 0.269386631 | 0.224 | 0.065 | 7.08E−18 |
| Cyp2d40 | 3.06E−22 | −0.321717407 | 0.181 | 0.35 | 7.61E−18 |
| Car5a | 3.09E−22 | −0.308769058 | 0.153 | 0.331 | 7.68E−18 |
| Cyp2c44 | 3.10E−22 | −0.372953076 | 0.48 | 0.698 | 7.70E−18 |
| Atp1b1 | 3.70E−22 | 0.370203015 | 0.391 | 0.226 | 9.18E−18 |
| Map3k5 | 3.76E−22 | 0.33471162 | 0.346 | 0.147 | 9.34E−18 |
| Ppp1r12a | 3.85E−22 | 0.356295305 | 0.317 | 0.13 | 9.55E−18 |
| Plk3 | 3.94E−22 | 0.416624571 | 0.413 | 0.226 | 9.77E−18 |
| Huwe1 | 4.05E−22 | 0.387931325 | 0.492 | 0.314 | 1.01E−17 |
| Zyx | 4.49E−22 | 0.298338034 | 0.263 | 0.09 | 1.11E−17 |
| Cep85l | 5.28E−22 | 0.277893187 | 0.267 | 0.089 | 1.31E−17 |
| Mup2 | 5.55E−22 | −0.50447051 | 0.21 | 0.392 | 1.38E−17 |
| Smdt1 | 5.76E−22 | −0.367317155 | 0.351 | 0.573 | 1.43E−17 |
| Sdhd | 5.80E−22 | −0.335261252 | 0.453 | 0.677 | 1.44E−17 |
| Psmb3 | 6.50E−22 | −0.330302519 | 0.153 | 0.345 | 1.62E−17 |
| Lrrfip2 | 7.11E−22 | 0.373099277 | 0.384 | 0.185 | 1.77E−17 |
| Oaz1 | 7.61E−22 | −0.347228062 | 0.532 | 0.75 | 1.89E−17 |
| Uqcc2 | 7.98E−22 | −0.326858302 | 0.189 | 0.386 | 1.98E−17 |
| Pnkd | 8.25E−22 | −0.292772175 | 0.153 | 0.327 | 2.05E−17 |
| Csde1 | 8.37E−22 | 0.315554781 | 0.632 | 0.577 | 2.08E−17 |
| Tcea3 | 8.56E−22 | −0.355007815 | 0.236 | 0.439 | 2.13E−17 |
| Ndufb8 | 8.67E−22 | −0.348076577 | 0.539 | 0.746 | 2.15E−17 |
| Kif21a | 9.22E−22 | 0.404170578 | 0.461 | 0.293 | 2.29E−17 |
| Bsg | 1.04E−21 | −0.31515012 | 0.58 | 0.797 | 2.59E−17 |
| 1810022K09Rik | 1.09E−21 | −0.300081452 | 0.107 | 0.301 | 2.71E−17 |
| Proc | 1.26E−21 | −0.270719362 | 0.678 | 0.874 | 3.12E−17 |
| Cldn2 | 1.28E−21 | −0.251076838 | 0.036 | 0.202 | 3.17E−17 |
| Acbd5 | 1.42E−21 | −0.3952179 | 0.492 | 0.708 | 3.52E−17 |
| Eif3c | 1.44E−21 | 0.301979757 | 0.604 | 0.541 | 3.57E−17 |
| Fkbp4 | 1.52E−21 | −0.353366286 | 0.341 | 0.544 | 3.77E−17 |
| Hibch | 1.61E−21 | −0.327130099 | 0.198 | 0.407 | 4.00E−17 |
| Derl2 | 1.82E−21 | −0.331734152 | 0.329 | 0.499 | 4.51E−17 |
| Tob1 | 1.82E−21 | −0.338705886 | 0.217 | 0.43 | 4.52E−17 |
| Coq9 | 2.44E−21 | −0.347936449 | 0.303 | 0.491 | 6.05E−17 |
| Nop10 | 2.50E−21 | −0.291908137 | 0.196 | 0.394 | 6.21E−17 |
| Dnajc19 | 2.66E−21 | −0.316006732 | 0.21 | 0.387 | 6.60E−17 |
| Rps9 | 2.66E−21 | −0.371793799 | 0.453 | 0.675 | 6.62E−17 |
| Anxa5 | 2.76E−21 | 0.365372347 | 0.391 | 0.239 | 6.84E−17 |
| Ubl5 | 2.91E−21 | −0.303313218 | 0.186 | 0.383 | 7.23E−17 |
| Paqr9 | 2.96E−21 | −0.332341635 | 0.217 | 0.41 | 7.36E−17 |
| Ndufa1 | 3.24E−21 | −0.364515751 | 0.332 | 0.561 | 8.05E−17 |
| Ndufv3 | 3.40E−21 | −0.397145193 | 0.36 | 0.593 | 8.44E−17 |
| Mcee | 3.44E−21 | −0.31991829 | 0.22 | 0.399 | 8.55E−17 |
| Rps3 | 3.96E−21 | −0.349320751 | 0.728 | 0.874 | 9.83E−17 |
| Pxk | 4.29E−21 | 0.281854648 | 0.303 | 0.116 | 1.07E−16 |
| Cela1 | 4.38E−21 | −0.272296838 | 0.129 | 0.304 | 1.09E−16 |
| Ankrd11 | 4.39E−21 | 0.415626852 | 0.389 | 0.198 | 1.09E−16 |
| C130074G19Rik | 4.71E−21 | −0.356338948 | 0.255 | 0.439 | 1.17E−16 |
| Arl6ip1 | 4.97E−21 | −0.364945533 | 0.42 | 0.643 | 1.23E−16 |
| Ces1f | 5.02E−21 | −0.312589958 | 0.618 | 0.817 | 1.25E−16 |
| Lsmd1 | 5.38E−21 | −0.313140806 | 0.167 | 0.357 | 1.34E−16 |
| Srsf11 | 5.96E−21 | 0.401209055 | 0.418 | 0.236 | 1.48E−16 |
| Csrp3 | 6.66E−21 | −0.293996249 | 0.098 | 0.256 | 1.65E−16 |
| Edf1 | 6.81E−21 | −0.381679393 | 0.492 | 0.703 | 1.69E−16 |
| Ebp | 7.00E−21 | −0.355328266 | 0.446 | 0.666 | 1.74E−16 |
| Eif4b | 7.04E−21 | 0.36816236 | 0.542 | 0.438 | 1.75E−16 |
| Ugt2a3 | 7.13E−21 | −0.307528085 | 0.558 | 0.773 | 1.77E−16 |
| Atf6 | 7.14E−21 | 0.377819844 | 0.432 | 0.273 | 1.77E−16 |
| Gm24245 | 7.16E−21 | 0.546463773 | 0.422 | 0.214 | 1.78E−16 |
| Nedd8 | 8.56E−21 | −0.356380686 | 0.298 | 0.506 | 2.13E−16 |
| AW112010 | 8.91E−21 | −0.298104828 | 0.229 | 0.435 | 2.21E−16 |
| Ndufs8 | 1.05E−20 | −0.361439192 | 0.391 | 0.607 | 2.60E−16 |
| Nucks1 | 1.28E−20 | 0.355785584 | 0.532 | 0.43 | 3.18E−16 |
| Cybb | 1.32E−20 | 0.377799564 | 0.107 | 0.031 | 3.27E−16 |
| Pacsin2 | 1.33E−20 | 0.370825053 | 0.37 | 0.201 | 3.31E−16 |
| Atp5o | 1.63E−20 | −0.331628292 | 0.487 | 0.712 | 4.05E−16 |
| Rbm25 | 1.80E−20 | 0.421459678 | 0.461 | 0.296 | 4.47E−16 |
| Pank1 | 1.90E−20 | −0.352650118 | 0.475 | 0.704 | 4.71E−16 |
| Il6ra | 2.05E−20 | 0.304392668 | 0.332 | 0.142 | 5.08E−16 |
| Dync1li1 | 2.16E−20 | 0.262406206 | 0.279 | 0.102 | 5.35E−16 |
| Baat | 2.16E−20 | −0.308582001 | 0.575 | 0.767 | 5.36E−16 |
| Slco1a1 | 2.33E−20 | −0.362843757 | 0.599 | 0.777 | 5.79E−16 |
| F10 | 2.54E−20 | −0.271936054 | 0.723 | 0.895 | 6.30E−16 |
| Bckdha | 2.54E−20 | −0.349979609 | 0.401 | 0.63 | 6.31E−16 |
| Grhpr | 2.65E−20 | −0.353058662 | 0.568 | 0.774 | 6.57E−16 |
| Mbl2 | 2.78E−20 | −0.346518253 | 0.673 | 0.829 | 6.92E−16 |
| AI182371 | 3.36E−20 | −0.319894937 | 0.403 | 0.622 | 8.34E−16 |
| Stk17b | 3.58E−20 | 0.291050054 | 0.126 | 0.028 | 8.90E−16 |
| Gcdh | 3.65E−20 | −0.300320098 | 0.668 | 0.85 | 9.07E−16 |
| Atf4 | 3.83E−20 | 0.341134734 | 0.58 | 0.442 | 9.51E−16 |
| Rnaseh2c | 3.85E−20 | −0.325683719 | 0.2 | 0.387 | 9.55E−16 |
| Fus | 3.95E−20 | 0.339324351 | 0.394 | 0.231 | 9.80E−16 |
| Sbno1 | 4.29E−20 | 0.367100512 | 0.394 | 0.228 | 1.07E−15 |
| Akap11 | 4.92E−20 | 0.346362286 | 0.351 | 0.164 | 1.22E−15 |
| Tmbim6 | 5.47E−20 | −0.271070995 | 0.852 | 0.942 | 1.36E−15 |
| Rsl1d1 | 5.47E−20 | 0.346056141 | 0.47 | 0.338 | 1.36E−15 |
| Hspa5 | 6.17E−20 | 0.268221373 | 0.914 | 0.944 | 1.53E−15 |
| Cebpb | 6.42E−20 | −0.361738286 | 0.286 | 0.45 | 1.59E−15 |
| Sar1b | 7.27E−20 | −0.307930709 | 0.568 | 0.772 | 1.80E−15 |
| Tmem242 | 7.52E−20 | −0.272538433 | 0.146 | 0.308 | 1.87E−15 |
| Selk | 8.09E−20 | −0.338314398 | 0.308 | 0.486 | 2.01E−15 |
| Psmb1 | 8.35E−20 | −0.358482353 | 0.575 | 0.759 | 2.07E−15 |
| Cxcl1 | 8.55E−20 | 0.370070418 | 0.444 | 0.314 | 2.12E−15 |
| Larp4b | 9.26E−20 | 0.34214672 | 0.453 | 0.281 | 2.30E−15 |
| Tm7sf2 | 9.70E−20 | −0.316381125 | 0.325 | 0.52 | 2.41E−15 |
| Pvrl2 | 1.04E−19 | −0.261260991 | 0.11 | 0.241 | 2.59E−15 |
| Pi4k2a | 1.06E−19 | 0.353561144 | 0.425 | 0.32 | 2.62E−15 |
| Gm4951 | 1.10E−19 | −0.322087312 | 0.284 | 0.491 | 2.73E−15 |
| Gm10800 | 1.24E−19 | 0.562597493 | 0.115 | 0.019 | 3.07E−15 |
| Mpc1 | 1.29E−19 | −0.280163495 | 0.203 | 0.369 | 3.21E−15 |
| Ctsc | 1.32E−19 | 0.361021766 | 0.403 | 0.271 | 3.29E−15 |
| Scaf11 | 1.37E−19 | 0.359845436 | 0.449 | 0.295 | 3.40E−15 |
| Bhmt2 | 1.46E−19 | −0.311413339 | 0.427 | 0.637 | 3.63E−15 |
| Dnajc5 | 1.48E−19 | 0.326483514 | 0.372 | 0.223 | 3.69E−15 |
| Mrpl12 | 1.58E−19 | −0.308200176 | 0.37 | 0.583 | 3.94E−15 |
| Anapc13 | 1.82E−19 | −0.364181956 | 0.253 | 0.427 | 4.52E−15 |
| Sdhb | 2.13E−19 | −0.289642823 | 0.604 | 0.797 | 5.30E−15 |
| Ndufb3 | 2.19E−19 | −0.327185415 | 0.246 | 0.43 | 5.45E−15 |
| Ptp4a1 | 3.01E−19 | 0.262691158 | 0.334 | 0.145 | 7.47E−15 |
| Rps21 | 3.54E−19 | −0.364450868 | 0.358 | 0.579 | 8.79E−15 |
| Fam214a | 3.55E−19 | −0.265769677 | 0.074 | 0.248 | 8.82E−15 |
| Dhx9 | 3.61E−19 | 0.350364065 | 0.427 | 0.252 | 8.96E−15 |
| Shb | 3.75E−19 | 0.315236194 | 0.382 | 0.204 | 9.32E−15 |
| 0610011F06Rik | 4.40E−19 | −0.303688511 | 0.425 | 0.653 | 1.09E−14 |
| Mdm2 | 4.71E−19 | 0.328296258 | 0.305 | 0.138 | 1.17E−14 |
| Nedd41 | 4.76E−19 | 0.281232817 | 0.332 | 0.147 | 1.18E−14 |
| Tmem106b | 4.85E−19 | 0.280204492 | 0.58 | 0.557 | 1.21E−14 |
| Bet1 | 5.02E−19 | −0.296563627 | 0.203 | 0.381 | 1.25E−14 |
| Rps10 | 5.49E−19 | −0.33690905 | 0.239 | 0.453 | 1.36E−14 |
| Myo1b | 5.63E−19 | 0.262427327 | 0.563 | 0.542 | 1.40E−14 |
| Kpna1 | 5.71E−19 | 0.32304803 | 0.363 | 0.206 | 1.42E−14 |
| Slc2a2 | 5.98E−19 | −0.312348392 | 0.263 | 0.479 | 1.48E−14 |
| Sdr9c7 | 6.14E−19 | −0.259988635 | 0.105 | 0.284 | 1.52E−14 |
| Hipk3 | 6.37E−19 | 0.339385214 | 0.396 | 0.223 | 1.58E−14 |
| Fam25c | 6.60E−19 | −0.297673676 | 0.158 | 0.356 | 1.64E−14 |
| Prdx5 | 6.78E−19 | −0.28208825 | 0.673 | 0.834 | 1.68E−14 |
| Otc | 8.10E−19 | −0.29903416 | 0.78 | 0.914 | 2.01E−14 |
| Apc | 8.43E−19 | 0.27896726 | 0.317 | 0.133 | 2.09E−14 |
| mmu-mir-6236 | 8.51E−19 | 0.520569164 | 0.831 | 0.837 | 2.11E−14 |
| Jhdm1d | 8.85E−19 | 0.304101624 | 0.279 | 0.112 | 2.20E−14 |
| Net1 | 8.95E−19 | 0.34785505 | 0.408 | 0.27 | 2.22E−14 |
| Romo1 | 9.12E−19 | −0.335854841 | 0.248 | 0.446 | 2.26E−14 |
| Sgk1 | 9.28E−19 | 0.284303028 | 0.21 | 0.085 | 2.30E−14 |
| Tmem160 | 9.41E−19 | −0.339044834 | 0.205 | 0.385 | 2.34E−14 |
| Acot13 | 9.58E−19 | −0.288638646 | 0.241 | 0.401 | 2.38E−14 |
| Sc4mol | 9.81E−19 | −0.326170476 | 0.444 | 0.642 | 2.44E−14 |
| Tbl1xr1 | 1.14E−18 | 0.307509115 | 0.341 | 0.176 | 2.84E−14 |
| Dync1h1 | 1.27E−18 | 0.379607116 | 0.379 | 0.21 | 3.17E−14 |
| Ak2 | 1.49E−18 | −0.345102135 | 0.458 | 0.66 | 3.71E−14 |
| Mrc1 | 1.63E−18 | 0.340444916 | 0.131 | 0.03 | 4.05E−14 |
| Slc41a2 | 1.74E−18 | 0.260152635 | 0.298 | 0.134 | 4.31E−14 |
| Fbxo21 | 1.75E−18 | −0.263087536 | 0.131 | 0.298 | 4.34E−14 |
| Mki67ip | 1.78E−18 | 0.318372964 | 0.344 | 0.171 | 4.41E−14 |
| Nudt4 | 1.91E−18 | 0.25418892 | 0.594 | 0.568 | 4.75E−14 |
| Mettl7a1 | 1.93E−18 | −0.322098553 | 0.465 | 0.682 | 4.79E−14 |
| Aox3 | 1.96E−18 | −0.333319828 | 0.764 | 0.894 | 4.88E−14 |
| 1110058L19Rik | 2.07E−18 | −0.258088587 | 0.129 | 0.281 | 5.15E−14 |
| Smap2 | 2.29E−18 | 0.348911433 | 0.363 | 0.184 | 5.69E−14 |
| Hmgcs1 | 2.30E−18 | −0.349112403 | 0.53 | 0.703 | 5.71E−14 |
| Apcs | 2.69E−18 | 0.298925674 | 0.551 | 0.525 | 6.67E−14 |
| Tmem86b | 2.75E−18 | −0.258236533 | 0.167 | 0.299 | 6.83E−14 |
| Txn1 | 2.82E−18 | −0.309123787 | 0.642 | 0.809 | 7.01E−14 |
| Mvd | 2.84E−18 | −0.270409867 | 0.112 | 0.269 | 7.04E−14 |
| Psmb6 | 3.03E−18 | −0.319377533 | 0.477 | 0.683 | 7.54E−14 |
| Cpb2 | 3.15E−18 | −0.321495225 | 0.635 | 0.813 | 7.81E−14 |
| Glyctk | 3.45E−18 | −0.297701904 | 0.251 | 0.466 | 8.56E−14 |
| Mrpl42 | 3.66E−18 | −0.284507519 | 0.215 | 0.386 | 9.09E−14 |
| Bnip3 | 3.73E−18 | −0.336168189 | 0.348 | 0.55 | 9.26E−14 |
| Slirp | 3.82E−18 | −0.308519262 | 0.205 | 0.39 | 9.48E−14 |
| 2010107E04Rik | 4.44E−18 | −0.318247806 | 0.473 | 0.681 | 1.10E−13 |
| Sec61b | 4.97E−18 | −0.290284119 | 0.232 | 0.437 | 1.24E−13 |
| Sigmar1 | 5.19E−18 | −0.305468332 | 0.353 | 0.514 | 1.29E−13 |
| Cyp2b10 | 5.48E−18 | −0.517460658 | 0.422 | 0.542 | 1.36E−13 |
| Mdh1 | 5.75E−18 | −0.307657821 | 0.747 | 0.887 | 1.43E−13 |
| Dhdh | 6.02E−18 | −0.334814135 | 0.377 | 0.583 | 1.49E−13 |
| Ptpn2 | 6.03E−18 | 0.273418437 | 0.255 | 0.097 | 1.50E−13 |
| Cyp2c55 | 6.35E−18 | −0.290435372 | 0.076 | 0.243 | 1.58E−13 |
| Cfhr1 | 6.80E−18 | −0.325332852 | 0.384 | 0.572 | 1.69E−13 |
| Bptf | 7.62E−18 | 0.324463857 | 0.332 | 0.155 | 1.89E−13 |
| Yap1 | 7.96E−18 | 0.328109852 | 0.375 | 0.207 | 1.98E−13 |
| Rexo2 | 8.64E−18 | −0.3263129 | 0.387 | 0.578 | 2.15E−13 |
| Akr1e1 | 9.43E−18 | −0.258207619 | 0.167 | 0.299 | 2.34E−13 |
| Tjp1 | 9.51E−18 | 0.286163863 | 0.251 | 0.096 | 2.36E−13 |
| Cyp4f15 | 9.51E−18 | −0.299840345 | 0.241 | 0.428 | 2.36E−13 |
| Naprt1 | 1.22E−17 | −0.338143058 | 0.317 | 0.492 | 3.03E−13 |
| Slco1a4 | 1.39E−17 | 0.313739505 | 0.387 | 0.238 | 3.44E−13 |
| Rab6a | 1.49E−17 | 0.32343226 | 0.461 | 0.335 | 3.69E−13 |
| Slc10a1 | 1.54E−17 | −0.325757408 | 0.695 | 0.843 | 3.82E−13 |
| Ipo7 | 1.64E−17 | 0.324580152 | 0.334 | 0.168 | 4.06E−13 |
| Cyp4f14 | 1.75E−17 | −0.295933211 | 0.387 | 0.6 | 4.35E−13 |
| Tnks2 | 1.84E−17 | 0.317626402 | 0.358 | 0.197 | 4.57E−13 |
| Macf1 | 1.95E−17 | 0.404177263 | 0.377 | 0.219 | 4.84E−13 |
| Esd | 2.01E−17 | −0.280760023 | 0.601 | 0.784 | 4.98E−13 |
| Bst2 | 2.09E−17 | −0.315565973 | 0.346 | 0.54 | 5.20E−13 |
| Stbd1 | 2.72E−17 | −0.273216024 | 0.181 | 0.362 | 6.74E−13 |
| Abcc2 | 2.91E−17 | 0.289681267 | 0.632 | 0.612 | 7.23E−13 |
| Aqp9 | 3.28E−17 | −0.310863805 | 0.434 | 0.647 | 8.15E−13 |
| Ddx3y | 3.39E−17 | 0.339517654 | 0.47 | 0.353 | 8.42E−13 |
| Pet100 | 3.40E−17 | −0.28003555 | 0.172 | 0.339 | 8.44E−13 |
| Agpat2 | 3.42E−17 | −0.298159013 | 0.437 | 0.638 | 8.49E−13 |
| Fah | 3.50E−17 | −0.259255187 | 0.652 | 0.834 | 8.70E−13 |
| Tmem147 | 3.77E−17 | −0.283785217 | 0.301 | 0.491 | 9.36E−13 |
| Sf1 | 3.87E−17 | 0.30230624 | 0.351 | 0.176 | 9.61E−13 |
| Ddah1 | 4.14E−17 | −0.312000429 | 0.301 | 0.482 | 1.03E−12 |
| Tmem37 | 4.76E−17 | −0.282207793 | 0.253 | 0.44 | 1.18E−12 |
| Dhrs4 | 5.12E−17 | −0.332074421 | 0.492 | 0.69 | 1.27E−12 |
| Mrp133 | 5.59E−17 | −0.265447876 | 0.241 | 0.419 | 1.39E−12 |
| Pik3c2a | 6.36E−17 | 0.324870962 | 0.341 | 0.198 | 1.58E−12 |
| Acads | 6.69E−17 | −0.32421998 | 0.341 | 0.539 | 1.66E−12 |
| D17Wsu92e | 7.50E−17 | 0.252482331 | 0.601 | 0.588 | 1.86E−12 |
| Coro1c | 7.89E−17 | 0.306679199 | 0.208 | 0.068 | 1.96E−12 |
| Aadat | 8.39E−17 | −0.318627951 | 0.329 | 0.507 | 2.08E−12 |
| Rlim | 8.53E−17 | 0.31023282 | 0.305 | 0.142 | 2.12E−12 |
| Sult1d1 | 8.95E−17 | −0.316458159 | 0.492 | 0.684 | 2.22E−12 |
| Mrpl34 | 9.07E−17 | −0.283750306 | 0.215 | 0.361 | 2.25E−12 |
| Tspo | 1.02E−16 | −0.315729205 | 0.313 | 0.506 | 2.54E−12 |
| Fdps | 1.07E−16 | −0.266347718 | 0.248 | 0.445 | 2.67E−12 |
| Sdsl | 1.11E−16 | 0.259290764 | 0.303 | 0.137 | 2.77E−12 |
| Nrp1 | 1.16E−16 | 0.360791537 | 0.391 | 0.253 | 2.88E−12 |
| 0610007P14Rik | 1.21E−16 | −0.257015045 | 0.184 | 0.346 | 3.00E−12 |
| Abcd3 | 1.26E−16 | −0.271805105 | 0.728 | 0.879 | 3.13E−12 |
| Etnk1 | 1.34E−16 | 0.315992963 | 0.442 | 0.328 | 3.33E−12 |
| Actn4 | 1.35E−16 | 0.312388865 | 0.53 | 0.425 | 3.35E−12 |
| Chmp4b | 1.39E−16 | 0.300153098 | 0.442 | 0.301 | 3.46E−12 |
| Gadd45gip1 | 1.50E−16 | −0.304090556 | 0.22 | 0.4 | 3.73E−12 |
| Mrpl11 | 1.53E−16 | −0.268482417 | 0.186 | 0.345 | 3.80E−12 |
| Ndufs4 | 1.68E−16 | −0.313160933 | 0.391 | 0.562 | 4.17E−12 |
| Tmem150a | 1.70E−16 | −0.253507791 | 0.172 | 0.335 | 4.21E−12 |
| Mrpl15 | 1.77E−16 | −0.291228504 | 0.236 | 0.415 | 4.39E−12 |
| Ipo5 | 1.94E−16 | 0.268735631 | 0.277 | 0.117 | 4.82E−12 |
| Atp2b1 | 2.09E−16 | 0.332096305 | 0.334 | 0.172 | 5.18E−12 |
| Erbb2ip | 2.14E−16 | 0.333163506 | 0.489 | 0.364 | 5.31E−12 |
| Amy1 | 2.20E−16 | −0.282967905 | 0.463 | 0.676 | 5.45E−12 |
| Mrpl41 | 2.22E−16 | −0.267412137 | 0.174 | 0.348 | 5.50E−12 |
| Kif1b | 2.36E−16 | 0.371764707 | 0.527 | 0.435 | 5.86E−12 |
| Birc6 | 2.38E−16 | 0.344367807 | 0.446 | 0.28 | 5.91E−12 |
| Agxt | 2.44E−16 | −0.258383387 | 0.601 | 0.789 | 6.06E−12 |
| Rpl13 | 2.49E−16 | −0.298816085 | 0.294 | 0.464 | 6.18E−12 |
| Pgls | 2.72E−16 | −0.274714156 | 0.22 | 0.345 | 6.74E−12 |
| Ndufv2 | 2.72E−16 | −0.327639406 | 0.461 | 0.648 | 6.75E−12 |
| Ndufb10 | 3.33E−16 | −0.300358086 | 0.489 | 0.681 | 8.26E−12 |
| Myeov2 | 3.91E−16 | −0.305189789 | 0.303 | 0.48 | 9.72E−12 |
| Supt6 | 4.06E−16 | 0.291294768 | 0.291 | 0.137 | 1.01E−11 |
| Cxcl12 | 4.35E−16 | −0.305838679 | 0.251 | 0.419 | 1.08E−11 |
| Aldh4a1 | 4.68E−16 | −0.29070878 | 0.425 | 0.613 | 1.16E−11 |
| Apof | 4.96E−16 | −0.252173807 | 0.759 | 0.897 | 1.23E−11 |
| Rap1b | 5.54E−16 | 0.340766029 | 0.406 | 0.258 | 1.38E−11 |
| Acaa1a | 5.73E−16 | −0.301848759 | 0.475 | 0.679 | 1.42E−11 |
| Pak2 | 5.87E−16 | 0.281467155 | 0.308 | 0.143 | 1.46E−11 |
| Tspan31 | 5.99E−16 | −0.295881364 | 0.415 | 0.622 | 1.49E−11 |
| Ubr4 | 6.49E−16 | 0.330144527 | 0.411 | 0.25 | 1.61E−11 |
| Bdh1 | 7.35E−16 | −0.293611347 | 0.315 | 0.49 | 1.82E−11 |
| Cap1 | 7.77E−16 | 0.325055169 | 0.356 | 0.202 | 1.93E−11 |
| Syncrip | 8.09E−16 | 0.27476476 | 0.365 | 0.198 | 2.01E−11 |
| Nrip1 | 8.50E−16 | 0.272854584 | 0.246 | 0.097 | 2.11E−11 |
| Tmprss6 | 8.97E−16 | −0.280234101 | 0.551 | 0.738 | 2.23E−11 |
| Commd3 | 9.66E−16 | −0.265556375 | 0.274 | 0.45 | 2.40E−11 |
| Il13ra1 | 1.20E−15 | 0.278527191 | 0.294 | 0.143 | 2.99E−11 |
| Tab2 | 1.24E−15 | 0.348412001 | 0.425 | 0.3 | 3.07E−11 |
| Bzw1 | 1.26E−15 | 0.294810535 | 0.475 | 0.344 | 3.12E−11 |
| Arhgap5 | 1.28E−15 | 0.264043476 | 0.506 | 0.454 | 3.18E−11 |
| Rpl14 | 1.45E−15 | −0.277637754 | 0.372 | 0.574 | 3.61E−11 |
| Clic4 | 1.62E−15 | 0.299795775 | 0.368 | 0.221 | 4.01E−11 |
| Lgals9 | 1.79E−15 | −0.269998113 | 0.621 | 0.793 | 4.44E−11 |
| Mrpl20 | 2.03E−15 | −0.309348635 | 0.301 | 0.489 | 5.03E−11 |
| C1d | 2.60E−15 | −0.255261652 | 0.208 | 0.354 | 6.45E−11 |
| Alcam | 2.79E−15 | 0.323788815 | 0.396 | 0.26 | 6.92E−11 |
| Mapk6 | 2.93E−15 | 0.256831906 | 0.303 | 0.145 | 7.27E−11 |
| Mrpl28 | 2.99E−15 | −0.269896048 | 0.229 | 0.378 | 7.42E−11 |
| Fam195a | 3.00E−15 | −0.264890901 | 0.205 | 0.373 | 7.46E−11 |
| Aes | 3.03E−15 | −0.264214993 | 0.535 | 0.729 | 7.53E−11 |
| Gstk1 | 3.20E−15 | −0.282802747 | 0.351 | 0.532 | 7.93E−11 |
| Snx10 | 3.26E−15 | 0.285642594 | 0.341 | 0.188 | 8.10E−11 |
| Plcxd2 | 3.73E−15 | −0.275192522 | 0.162 | 0.332 | 9.27E−11 |
| Park7 | 3.77E−15 | −0.260230015 | 0.601 | 0.784 | 9.36E−11 |
| Med13 | 4.26E−15 | 0.294918978 | 0.325 | 0.168 | 1.06E−10 |
| Btaf1 | 4.29E−15 | 0.275463861 | 0.284 | 0.138 | 1.07E−10 |
| Spcs1 | 4.65E−15 | −0.26287037 | 0.236 | 0.413 | 1.16E−10 |
| Abcf1 | 5.59E−15 | 0.303190272 | 0.458 | 0.315 | 1.39E−10 |
| Selo | 5.68E−15 | −0.257421903 | 0.217 | 0.381 | 1.41E−10 |
| Usp9x | 5.80E−15 | 0.318385696 | 0.451 | 0.299 | 1.44E−10 |
| Klf3 | 5.86E−15 | 0.267357487 | 0.301 | 0.146 | 1.45E−10 |
| Iah1 | 5.99E−15 | −0.292985785 | 0.446 | 0.612 | 1.49E−10 |
| Il4ra | 6.47E−15 | 0.25739098 | 0.248 | 0.102 | 1.61E−10 |
| Acat3 | 6.81E−15 | −0.311510623 | 0.427 | 0.6 | 1.69E−10 |
| Ugt3a2 | 7.27E−15 | −0.258613351 | 0.492 | 0.689 | 1.80E−10 |
| PM20l1 | 8.41E−15 | 0.308488005 | 0.372 | 0.212 | 2.09E−10 |
| Abcb11 | 8.50E−15 | −0.261778471 | 0.618 | 0.791 | 2.11E−10 |
| Pkp4 | 8.99E−15 | 0.323033636 | 0.401 | 0.258 | 2.23E−10 |
| Cbr1 | 1.14E−14 | 0.284717454 | 0.494 | 0.395 | 2.84E−10 |
| Deb1 | 1.16E−14 | −0.267029156 | 0.274 | 0.443 | 2.87E−10 |
| Akr1d1 | 1.31E−14 | −0.278865899 | 0.332 | 0.515 | 3.24E−10 |
| Rangap1 | 1.35E−14 | 0.296226768 | 0.387 | 0.244 | 3.36E−10 |
| Rmnd5a | 1.44E−14 | 0.318903459 | 0.47 | 0.352 | 3.57E−10 |
| Psma6 | 1.49E−14 | −0.30353658 | 0.501 | 0.675 | 3.71E−10 |
| Nqo2 | 1.50E−14 | −0.267802053 | 0.317 | 0.486 | 3.73E−10 |
| Rpl34 | 1.53E−14 | −0.297598539 | 0.246 | 0.398 | 3.81E−10 |
| Rnf169 | 1.61E−14 | 0.333974467 | 0.315 | 0.173 | 4.00E−10 |
| Hpn | 1.64E−14 | −0.268601326 | 0.406 | 0.604 | 4.07E−10 |
| Plin5 | 1.64E−14 | 0.296281512 | 0.456 | 0.315 | 4.07E−10 |
| Abcg2 | 1.68E−14 | −0.280923445 | 0.294 | 0.466 | 4.17E−10 |
| Pa2g4 | 1.82E−14 | 0.289349263 | 0.492 | 0.418 | 4.53E−10 |
| Sf3b1 | 1.91E−14 | 0.28884767 | 0.508 | 0.406 | 4.75E−10 |
| Serpina3c | 1.99E−14 | −0.287011328 | 0.258 | 0.385 | 4.94E−10 |
| Swi5 | 2.17E−14 | −0.262086804 | 0.341 | 0.501 | 5.38E−10 |
| Hsbp1 | 2.27E−14 | −0.286726926 | 0.329 | 0.498 | 5.65E−10 |
| Sema4g | 2.43E−14 | −0.250459516 | 0.27 | 0.451 | 6.04E−10 |
| Gtf2a2 | 2.63E−14 | 0.279207862 | 0.379 | 0.237 | 6.54E−10 |
| Gcat | 2.78E−14 | −0.258728349 | 0.315 | 0.49 | 6.91E−10 |
| Ccdc50 | 2.83E−14 | 0.285830087 | 0.401 | 0.246 | 7.02E−10 |
| Ldlr | 2.90E−14 | 0.282025946 | 0.432 | 0.364 | 7.20E−10 |
| Srrm2 | 3.29E−14 | 0.328159189 | 0.456 | 0.313 | 8.16E−10 |
| Suco | 3.46E−14 | 0.250962583 | 0.26 | 0.113 | 8.60E−10 |
| Mapkapk2 | 3.50E−14 | 0.31382654 | 0.396 | 0.254 | 8.68E−10 |
| Tbca | 3.66E−14 | −0.27261031 | 0.284 | 0.401 | 9.08E−10 |
| Slc26a1 | 3.68E−14 | −0.28165635 | 0.329 | 0.495 | 9.13E−10 |
| Acsm5 | 3.73E−14 | −0.261394035 | 0.236 | 0.397 | 9.27E−10 |
| Eif3a | 3.77E−14 | 0.314713983 | 0.542 | 0.461 | 9.36E−10 |
| Mrps12 | 4.19E−14 | −0.250441318 | 0.193 | 0.332 | 1.04E−09 |
| St3gal4 | 4.22E−14 | 0.275033534 | 0.368 | 0.244 | 1.05E−09 |
| mt-Atp6 | 4.35E−14 | −0.47818742 | 0.305 | 0.452 | 1.08E−09 |
| Dera | 4.65E−14 | −0.267441985 | 0.32 | 0.49 | 1.15E−09 |
| Slc25a10 | 4.71E−14 | −0.282805644 | 0.351 | 0.536 | 1.17E−09 |
| Plbd2 | 4.76E−14 | 0.264873115 | 0.344 | 0.199 | 1.18E−09 |
| Hectd1 | 5.23E−14 | 0.30802968 | 0.547 | 0.476 | 1.30E−09 |
| Srd5a1 | 6.05E−14 | −0.277347272 | 0.308 | 0.464 | 1.50E−09 |
| Ubb | 6.13E−14 | −0.275030382 | 0.265 | 0.45 | 1.52E−09 |
| Mlycd | 7.23E−14 | −0.290948387 | 0.329 | 0.503 | 1.80E−09 |
| Cep85 | 7.37E−14 | 0.28389234 | 0.313 | 0.167 | 1.83E−09 |
| Pdzd8 | 8.19E−14 | 0.286270568 | 0.36 | 0.206 | 2.03E−09 |
| Ddx27 | 8.44E−14 | 0.252155421 | 0.286 | 0.133 | 2.09E−09 |
| Kdm5a | 8.92E−14 | 0.351390096 | 0.308 | 0.157 | 2.22E−09 |
| Ptpn11 | 1.04E−13 | 0.277804552 | 0.37 | 0.218 | 2.58E−09 |
| Trib1 | 1.05E−13 | 0.331693479 | 0.411 | 0.308 | 2.61E−09 |
| 2810428I15Rik | 1.10E−13 | −0.263117928 | 0.2 | 0.349 | 2.74E−09 |
| Sptan1 | 1.23E−13 | 0.312663889 | 0.348 | 0.21 | 3.05E−09 |
| Gldc | 1.23E−13 | 0.280156601 | 0.465 | 0.375 | 3.07E−09 |
| Impad1 | 1.37E−13 | 0.32267465 | 0.391 | 0.263 | 3.41E−09 |
| Gch1 | 1.38E−13 | −0.269053605 | 0.461 | 0.643 | 3.42E−09 |
| Rps24 | 1.49E−13 | −0.262473665 | 0.453 | 0.637 | 3.71E−09 |
| Dcaf11 | 1.51E−13 | −0.281659388 | 0.446 | 0.617 | 3.76E−09 |
| Rraga | 1.66E−13 | 0.262613483 | 0.337 | 0.196 | 4.13E−09 |
| Furin | 2.01E−13 | 0.253587917 | 0.477 | 0.417 | 4.99E−09 |
| Golga4 | 2.01E−13 | 0.352084678 | 0.427 | 0.31 | 5.00E−09 |
| Ppp2r5a | 2.19E−13 | −0.260504538 | 0.43 | 0.594 | 5.43E−09 |
| Arpc1b | 2.20E−13 | 0.259676842 | 0.263 | 0.119 | 5.46E−09 |
| Acsl4 | 2.24E−13 | 0.298529302 | 0.427 | 0.323 | 5.55E−09 |
| Lipc | 2.28E−13 | −0.254841484 | 0.401 | 0.59 | 5.65E−09 |
| Diap1 | 3.27E−13 | 0.325941798 | 0.382 | 0.241 | 8.12E−09 |
| Rabac1 | 3.43E−13 | −0.265987205 | 0.346 | 0.524 | 8.51E−09 |
| Pmpcb | 3.44E−13 | −0.263615722 | 0.356 | 0.536 | 8.55E−09 |
| Mgea5 | 3.67E−13 | 0.32630901 | 0.413 | 0.268 | 9.10E−09 |
| Slc16a10 | 3.80E−13 | −0.254826191 | 0.196 | 0.315 | 9.44E−09 |
| Ppara | 3.86E−13 | −0.287133273 | 0.234 | 0.407 | 9.59E−09 |
| Usp4 | 4.19E−13 | 0.279964381 | 0.439 | 0.286 | 1.04E−08 |
| Ptpmt1 | 4.26E−13 | −0.255981462 | 0.372 | 0.546 | 1.06E−08 |
| Psma1 | 4.43E−13 | −0.256671259 | 0.353 | 0.521 | 1.10E−08 |
| Ythdc1 | 4.81E−13 | 0.264130576 | 0.301 | 0.15 | 1.19E−08 |
| Abce1 | 4.88E−13 | 0.279955892 | 0.437 | 0.31 | 1.21E−08 |
| Ppip5k2 | 4.98E−13 | 0.320939415 | 0.344 | 0.197 | 1.24E−08 |
| Ftsj3 | 5.57E−13 | 0.255227431 | 0.346 | 0.191 | 1.38E−08 |
| Tpi1 | 5.58E−13 | −0.26157943 | 0.539 | 0.706 | 1.38E−08 |
| Slc16a2 | 5.92E−13 | −0.251969763 | 0.255 | 0.412 | 1.47E−08 |
| Ubr5 | 6.17E−13 | 0.305718363 | 0.399 | 0.248 | 1.53E−08 |
| Il6st | 6.32E−13 | 0.316786986 | 0.411 | 0.273 | 1.57E−08 |
| Xrn2 | 6.73E−13 | 0.298471923 | 0.387 | 0.245 | 1.67E−08 |
| Cdh1 | 6.96E−13 | 0.269696738 | 0.327 | 0.202 | 1.73E−08 |
| Nek7 | 9.74E−13 | 0.291098417 | 0.394 | 0.271 | 2.42E−08 |
| Slc25a51 | 9.95E−13 | 0.263985158 | 0.308 | 0.161 | 2.47E−08 |
| Magi3 | 1.02E−12 | 0.269961841 | 0.303 | 0.156 | 2.54E−08 |
| Pde3b | 1.14E−12 | −0.258970093 | 0.243 | 0.376 | 2.82E−08 |
| Megf9 | 1.14E−12 | 0.276890033 | 0.289 | 0.158 | 2.82E−08 |
| Zfp106 | 1.96E−12 | 0.336920255 | 0.391 | 0.254 | 4.87E−08 |
| Gstm3 | 2.40E−12 | −0.411092337 | 0.265 | 0.414 | 5.97E−08 |
| Sars | 2.65E−12 | 0.276853119 | 0.43 | 0.322 | 6.58E−08 |
| Psap | 3.18E−12 | 0.257800256 | 0.556 | 0.513 | 7.90E−08 |
| Trip11 | 3.20E−12 | 0.347886673 | 0.341 | 0.2 | 7.94E−08 |
| Naa50 | 3.28E−12 | 0.265659058 | 0.444 | 0.358 | 8.14E−08 |
| Prpf19 | 3.66E−12 | 0.25876049 | 0.432 | 0.315 | 9.09E−08 |
| Pcm1 | 4.11E−12 | 0.265397644 | 0.274 | 0.142 | 1.02E−07 |
| Papss2 | 4.45E−12 | −0.254475327 | 0.337 | 0.507 | 1.10E−07 |
| Coasy | 5.10E−12 | −0.253428655 | 0.327 | 0.475 | 1.27E−07 |
| Actr3 | 5.91E−12 | 0.275997927 | 0.36 | 0.233 | 1.47E−07 |
| Ccnl1 | 6.84E−12 | 0.256623048 | 0.351 | 0.206 | 1.70E−07 |
| Hipk1 | 6.86E−12 | 0.291600227 | 0.337 | 0.195 | 1.70E−07 |
| Riok3 | 6.99E−12 | 0.291562038 | 0.43 | 0.345 | 1.74E−07 |
| Pdia4 | 7.20E−12 | −0.27191836 | 0.43 | 0.595 | 1.79E−07 |
| Nploc4 | 7.73E−12 | 0.291804954 | 0.351 | 0.214 | 1.92E−07 |
| Dsp | 8.13E−12 | 0.265533344 | 0.32 | 0.178 | 2.02E−07 |
| Spag9 | 8.42E−12 | 0.318820155 | 0.415 | 0.286 | 2.09E−07 |
| Map3k2 | 9.41E−12 | 0.251791147 | 0.265 | 0.131 | 2.34E−07 |
| Gars | 1.75E−11 | 0.257638792 | 0.334 | 0.199 | 4.33E−07 |
| Arhgdia | 1.94E−11 | 0.256326455 | 0.317 | 0.186 | 4.81E−07 |
| Gcgr | 2.44E−11 | −0.257636913 | 0.446 | 0.609 | 6.06E−07 |
| Eif4ebp1 | 3.67E−11 | 0.261682735 | 0.387 | 0.283 | 9.11E−07 |
| Sco2 | 3.81E−11 | −0.260508515 | 0.353 | 0.508 | 9.46E−07 |
| Zfp259 | 3.89E−11 | 0.255636639 | 0.32 | 0.182 | 9.66E−07 |
| Rbm39 | 3.97E−11 | 0.265732671 | 0.506 | 0.42 | 9.85E−07 |
| Zfand5 | 4.18E−11 | 0.28815165 | 0.401 | 0.293 | 1.04E−06 |
| Usp7 | 4.52E−11 | 0.287320099 | 0.363 | 0.241 | 1.12E−06 |
| Eif4a1 | 5.11E−11 | 0.251906299 | 0.418 | 0.291 | 1.27E−06 |
| Herc1 | 6.98E−11 | 0.256188826 | 0.246 | 0.122 | 1.73E−06 |
| Gm5424 | 1.56E−10 | 0.269800095 | 0.411 | 0.3 | 3.88E−06 |
| Ndufa5 | 1.58E−10 | −0.251988986 | 0.418 | 0.567 | 3.93E−06 |
| Ctnnd1 | 2.19E−10 | 0.251848095 | 0.358 | 0.217 | 5.45E−06 |
| Cul3 | 2.70E−10 | 0.278802183 | 0.415 | 0.303 | 6.69E−06 |
| 4932438A13Rik | 2.79E−10 | 0.287850722 | 0.329 | 0.203 | 6.93E−06 |
| Usp34 | 2.86E−10 | 0.285942387 | 0.37 | 0.243 | 7.10E−06 |
| Ppm1g | 3.21E−10 | 0.252225215 | 0.358 | 0.221 | 7.96E−06 |
| Smoc1 | 3.30E−10 | 0.26157412 | 0.368 | 0.238 | 8.20E−06 |
| Erf3b | 3.93E−10 | 0.250328103 | 0.449 | 0.366 | 9.77E−06 |
| Gadd45a | 4.63E−10 | 0.282867411 | 0.251 | 0.149 | 1.15E−05 |
| Cul1 | 4.87E−10 | 0.259206487 | 0.425 | 0.313 | 1.21E−05 |
| Eea1 | 5.07E−10 | 0.261201113 | 0.363 | 0.245 | 1.26E−05 |
| Tln1 | 5.68E−10 | 0.321033046 | 0.31 | 0.202 | 1.41E−05 |
| Rassf3 | 6.24E−10 | 0.291236708 | 0.422 | 0.319 | 1.55E−05 |
| Herc4 | 8.92E−10 | 0.267474882 | 0.332 | 0.217 | 2.21E−05 |
| 4931406C07Rik | 9.77E−10 | −0.254363633 | 0.616 | 0.748 | 2.43E−05 |
| Slc30a1 | 1.09E−09 | 0.250570123 | 0.346 | 0.238 | 2.72E−05 |
| Gstp1 | 1.77E−09 | −0.264374672 | 0.685 | 0.822 | 4.40E−05 |
| Bclaf1 | 3.63E−09 | 0.26848767 | 0.372 | 0.256 | 9.01E−05 |
| Gnai2 | 6.70E−09 | 0.281667665 | 0.391 | 0.31 | 0.000166446 |
| Gm20594 | 1.18E−08 | 0.295686612 | 0.442 | 0.294 | 0.000292421 |
| Ash1l | 1.18E−08 | 0.265693821 | 0.301 | 0.175 | 0.000293692 |
| Rock1 | 1.47E−07 | 0.2607635 | 0.413 | 0.308 | 0.003638685 |
| Gda | 9.30E−07 | 0.266276129 | 0.243 | 0.165 | 0.02310523 |
| Ftl1 | 1.58E−06 | −0.26029441 | 0.317 | 0.409 | 0.039229795 |
| Gm23935 | 5.53E−06 | 0.424222263 | 0.878 | 0.925 | 0.137419547 |

TABLE 3
Differentially expressed genes between time point A24 and untreated (UT).

| ID | p_val | avg_logFC | pct.1 | pct.2 | p_val_adj |
|---|---|---|---|---|---|
| Gm15564 | 0 | 1.820580657 | 0.982 | 0.956 | 0 |
| Lars2 | 0 | 1.711400221 | 0.998 | 0.994 | 0 |
| Gm26924 | 0 | 1.419027502 | 1 | 1 | 0 |
| Sephs2 | 0 | 0.889992859 | 0.949 | 0.761 | 0 |
| Grip2 | 0 | 0.877868293 | 0.475 | 0.002 | 0 |
| Fgl1 | 0 | 0.864933885 | 0.938 | 0.784 | 0 |
| Fga | 0 | 0.828543313 | 0.998 | 0.997 | 0 |
| Fgb | 0 | 0.744973762 | 1 | 1 | 0 |
| Fgg | 0 | 0.680530925 | 0.999 | 0.991 | 0 |
| Apoa2 | 0 | −0.545518454 | 0.997 | 1 | 0 |
| mt-Rnr2 | 0 | −0.778341998 | 1 | 1 | 0 |
| Mup3 | 0 | −0.806219448 | 0.998 | 1 | 0 |
| mt-Nd2 | 0 | −1.241926048 | 0.949 | 0.999 | 0 |
| mt-Nd5 | 0 | −1.383591054 | 0.869 | 0.995 | 0 |
| mt-Nd1 | 0 | −1.419683574 | 0.942 | 1 | 0 |
| mt-Co1 | 0 | −1.43342755 | 0.949 | 0.999 | 0 |
| mt-Cytb | 0 | −1.571232911 | 0.926 | 0.999 | 0 |
| Mup11 | 0 | −1.611434806 | 0.873 | 1 | 0 |
| Mup19 | 0 | −1.699352699 | 0.916 | 1 | 0 |
| Mup18 | 0 | −1.727178347 | 0.769 | 0.997 | 0 |
| Mup17 | 0 | −1.800348604 | 0.938 | 1 | 0 |
| Mup9 | 0 | −1.847446915 | 0.619 | 0.976 | 0 |
| mt-Nd4 | 0 | −1.957424825 | 0.849 | 0.998 | 0 |
| Ankrd55 | 2.80898674204367e−314 | −1.8735659 | 0.11 | 0.696 | 6.97527587784284e−310 |
| Acaa1b | 4.58424219320629e−311 | −0.85084133 | 0.757 | 0.969 | 1.14E−306 |
| mt-Co2 | 2.73E−303 | −1.396543228 | 0.034 | 0.629 | 6.78E−299 |
| Serpina1a | 1.18E−299 | −0.675201963 | 0.924 | 0.991 | 2.93E−295 |
| n-R5-8s1 | 6.16E−298 | 1.252818829 | 0.522 | 0.031 | 1.53E−293 |
| Gpx1 | 2.33E−291 | −0.50797139 | 0.977 | 0.999 | 5.78E−287 |
| Apoe | 1.69E−261 | −0.421271729 | 1 | 1 | 4.18E−257 |
| Mup6 | 1.39E−251 | −1.678184828 | 0.795 | 0.992 | 3.44E−247 |
| Cyb5 | 3.79E−249 | −0.704808492 | 0.955 | 0.983 | 9.41E−245 |
| Gm24601 | 1.30E−237 | 0.984391691 | 0.404 | 0.015 | 3.24E−233 |
| Sepp1 | 6.71E−231 | 0.448586822 | 1 | 1 | 1.67E−226 |
| Mup16 | 6.04E−222 | −1.018975936 | 0.615 | 0.934 | 1.50E−217 |
| Hyou1 | 1.76E−219 | 0.846484822 | 0.717 | 0.385 | 4.36E−215 |
| Gm13775 | 1.79E−215 | −1.032451941 | 0.306 | 0.761 | 4.43E−211 |
| mt-Co3 | 2.31E−212 | −1.167027737 | 0.115 | 0.603 | 5.73E−208 |
| Serpina1d | 3.91E−207 | −0.494007182 | 0.946 | 0.988 | 9.71E−203 |
| Hspa5 | 9.23E−204 | 0.610549064 | 0.974 | 0.944 | 2.29E−199 |
| Ugt2b5 | 1.22E−199 | −0.576498765 | 0.893 | 0.959 | 3.03E−195 |
| Gsta3 | 6.18E−189 | −0.66935487 | 0.925 | 0.956 | 1.54E−184 |
| Cyp2d9 | 1.16E−183 | −0.567166215 | 0.896 | 0.959 | 2.89E−179 |
| Mup15 | 4.07E−182 | −1.234928375 | 0.173 | 0.617 | 1.01E−177 |
| Pdia3 | 8.53E−181 | 0.593769188 | 0.895 | 0.797 | 2.12E−176 |
| Mup5 | 5.23E−180 | −1.370621307 | 0.47 | 0.839 | 1.30E−175 |
| Apoc3 | 7.62E−179 | −0.440433124 | 0.952 | 0.993 | 1.89E−174 |
| Serpina1c | 1.69E−176 | −0.402626732 | 0.983 | 0.998 | 4.19E−172 |
| Calr | 1.06E−174 | 0.555145472 | 0.952 | 0.885 | 2.64E−170 |
| Cyp2b10 | 5.60E−174 | −1.164490931 | 0.174 | 0.542 | 1.39E−169 |
| Hamp | 6.04E−172 | −0.826148443 | 0.552 | 0.884 | 1.50E−167 |
| Hsp90b1 | 1.49E−166 | 0.534208217 | 0.954 | 0.953 | 3.69E−162 |
| Fn1 | 5.06E−157 | 0.699634526 | 0.905 | 0.821 | 1.26E−152 |
| Scd1 | 6.56E−157 | 0.726687286 | 0.98 | 0.901 | 1.63E−152 |
| Uqcrq | 9.90E−156 | −0.595921048 | 0.856 | 0.951 | 2.46E−151 |
| Itih4 | 2.62E−154 | 0.525074867 | 0.974 | 0.948 | 6.50E−150 |
| Cyp3a11 | 8.42E−152 | −0.363891177 | 0.99 | 1 | 2.09E−147 |
| Apoc1 | 4.19E−149 | −0.380336769 | 0.976 | 0.999 | 1.04E−144 |
| Hp | 1.00E−142 | 0.431328341 | 0.998 | 0.999 | 2.49E−138 |
| Elovl3 | 2.48E−142 | −0.675967012 | 0.54 | 0.827 | 6.15E−138 |
| Mup4 | 1.89E−139 | −1.087038527 | 0.616 | 0.898 | 4.70E−135 |
| mt-Rnr1 | 7.20E−137 | −0.456211361 | 1 | 1 | 1.79E−132 |
| Bhmt | 8.82E−137 | −0.520184894 | 0.868 | 0.956 | 2.19E−132 |
| Mup21 | 6.83E−134 | −0.697735774 | 0.706 | 0.929 | 1.70E−129 |
| Serpina10 | 5.36E−129 | 0.565046404 | 0.813 | 0.641 | 1.33E−124 |
| Ahcy | 3.96E−128 | −0.562968597 | 0.73 | 0.916 | 9.83E−124 |
| Hdlbp | 4.80E−125 | 0.565147241 | 0.853 | 0.713 | 1.19E−120 |
| Apoc4 | 3.10E−122 | −0.428496936 | 0.944 | 0.98 | 7.71E−118 |
| Gnmt | 7.88E−122 | −0.46036106 | 0.972 | 0.988 | 1.96E−117 |
| Cox4i1 | 8.25E−122 | −0.454243631 | 0.904 | 0.958 | 2.05E−117 |
| Ssr3 | 7.84E−120 | 0.521769281 | 0.765 | 0.596 | 1.95E−115 |
| Glud1 | 1.95E−119 | 0.451048591 | 0.958 | 0.905 | 4.85E−115 |
| P4hb | 4.64E−119 | 0.405223744 | 0.973 | 0.941 | 1.15E−114 |
| Itih3 | 4.97E−115 | 0.492908293 | 0.931 | 0.89 | 1.23E−110 |
| Cyp4a10 | 1.47E−114 | −0.742207081 | 0.322 | 0.657 | 3.66E−110 |
| Saa2 | 5.09E−114 | 0.964488054 | 0.52 | 0.222 | 1.26E−109 |
| Apcs | 6.96E−112 | 0.606730845 | 0.738 | 0.525 | 1.73E−107 |
| Creld2 | 1.82E−109 | 0.608652695 | 0.583 | 0.296 | 4.53E−105 |
| Myh9 | 1.48E−108 | 0.6391137 | 0.595 | 0.318 | 3.67E−104 |
| Tmem205 | 7.63E−107 | −0.472107677 | 0.816 | 0.934 | 1.89E−102 |
| Egfr | 3.14E−106 | 0.504957782 | 0.882 | 0.809 | 7.81E−102 |
| C8g | 4.37E−106 | −0.481964476 | 0.79 | 0.91 | 1.09E−101 |
| Serpina1b | 2.55E−105 | −0.301770579 | 0.984 | 0.996 | 6.34E−101 |
| Ces1d | 9.18E−104 | −0.428906017 | 0.797 | 0.923 | 2.28E−99 |
| C3 | 1.35E−103 | 0.449521065 | 0.996 | 0.99 | 3.34E−99 |
| Cyp8b1 | 4.96E−103 | −0.553616206 | 0.489 | 0.745 | 1.23E−98 |
| B2m | 3.29E−102 | −0.417973886 | 0.905 | 0.963 | 8.16E−98 |
| Dnajc3 | 8.00E−101 | 0.45638751 | 0.85 | 0.772 | 1.99E−96 |
| Eif4g2 | 8.04E−99 | 0.465990267 | 0.8 | 0.678 | 2.00E−94 |
| Mup13 | 8.39E−99 | −0.694889672 | 0.257 | 0.546 | 2.08E−94 |
| Rnase4 | 1.83E−98 | 0.517836403 | 0.963 | 0.921 | 4.54E−94 |
| Rps27l | 2.39E−98 | −0.548506218 | 0.658 | 0.849 | 5.93E−94 |
| Iigp1 | 2.98E−97 | 0.454178892 | 0.832 | 0.759 | 7.41E−93 |
| Gadd45g | 3.17E−97 | −0.614019025 | 0.311 | 0.639 | 7.86E−93 |
| Eef1a1 | 4.27E−96 | 0.439251983 | 0.919 | 0.85 | 1.06E−91 |
| Pdia4 | 5.79E−95 | 0.488166898 | 0.733 | 0.595 | 1.44E−90 |
| Hnf4a | 7.91E−95 | 0.454905579 | 0.85 | 0.743 | 1.96E−90 |
| Arrdc3 | 1.26E−94 | 0.526871989 | 0.52 | 0.213 | 3.13E−90 |
| Cyp2c29 | 9.40E−93 | −0.586003377 | 0.709 | 0.93 | 2.33E−88 |
| Cth | 1.96E−92 | −0.510899065 | 0.708 | 0.838 | 4.86E−88 |
| Orm1 | 3.67E−90 | 0.365144434 | 0.963 | 0.952 | 9.11E−86 |
| Actb | 1.97E−88 | 0.38446276 | 0.919 | 0.886 | 4.90E−84 |
| Hint1 | 4.47E−88 | −0.473665273 | 0.691 | 0.858 | 1.11E−83 |
| Serpina1e | 8.25E−88 | −0.284344569 | 0.993 | 0.999 | 2.05E−83 |
| Pgrmc1 | 9.39E−88 | 0.350152674 | 0.953 | 0.94 | 2.33E−83 |
| AI182371 | 1.18E−87 | 0.447854829 | 0.747 | 0.622 | 2.93E−83 |
| Pzp | 2.47E−87 | 0.41600896 | 0.984 | 0.986 | 6.13E−83 |
| Oat | 3.36E−87 | −0.521209343 | 0.263 | 0.611 | 8.34E−83 |
| Ces3a | 3.37E−86 | −0.36873562 | 0.884 | 0.967 | 8.38E−82 |
| Calm1 | 3.86E−86 | 0.413146039 | 0.794 | 0.722 | 9.59E−82 |
| Sod1 | 6.37E−86 | −0.303629074 | 0.935 | 0.983 | 1.58E−81 |
| Gm26917 | 2.48E−84 | 0.823736053 | 0.391 | 0.181 | 6.16E−80 |
| Adh1 | 1.27E−83 | −0.342018972 | 0.942 | 0.96 | 3.15E−79 |
| Elovl2 | 6.28E−83 | 0.45224665 | 0.826 | 0.699 | 1.56E−78 |
| Cox6c | 1.19E−82 | −0.502802751 | 0.724 | 0.884 | 2.96E−78 |
| Pdcd4 | 2.20E−82 | 0.48815423 | 0.677 | 0.498 | 5.46E−78 |
| Hmgcs2 | 6.68E−82 | −0.317281221 | 0.951 | 0.986 | 1.66E−77 |
| Atp5h | 9.15E−82 | −0.579498914 | 0.45 | 0.682 | 2.27E−77 |
| Csad | 1.94E−80 | −0.531252493 | 0.469 | 0.744 | 4.82E−76 |
| Manf | 2.07E−80 | 0.468881688 | 0.632 | 0.44 | 5.14E−76 |
| Ech1 | 2.78E−80 | −0.429132615 | 0.715 | 0.856 | 6.90E−76 |
| Ces1c | 9.71E−80 | −0.278292823 | 0.985 | 0.985 | 2.41E−75 |
| Lrg1 | 1.78E−78 | 0.436838294 | 0.849 | 0.736 | 4.43E−74 |
| Insig1 | 1.09E−76 | 0.465534816 | 0.626 | 0.426 | 2.72E−72 |
| Steap4 | 1.33E−76 | 0.475973731 | 0.481 | 0.222 | 3.30E−72 |
| Gm24270 | 1.88E−76 | 0.26796863 | 0.145 | 0.006 | 4.67E−72 |
| Hc | 3.46E−74 | 0.367935082 | 0.905 | 0.903 | 8.59E−70 |
| Cyp2e1 | 9.55E−74 | −0.499033632 | 0.688 | 0.911 | 2.37E−69 |
| Nr1d2 | 2.65E−71 | 0.430636791 | 0.442 | 0.179 | 6.58E−67 |
| 1110008F13Rik | 3.48E−71 | −0.455270589 | 0.427 | 0.648 | 8.65E−67 |
| Canx | 6.16E−71 | 0.343383207 | 0.872 | 0.835 | 1.53E−66 |
| Cfh | 9.84E−71 | 0.400919606 | 0.921 | 0.897 | 2.44E−66 |
| Ndufa4 | 2.17E−70 | −0.443311137 | 0.766 | 0.889 | 5.38E−66 |
| Cebpb | 3.92E−70 | −0.471579534 | 0.216 | 0.45 | 9.72E−66 |
| Dhcr24 | 5.03E−70 | 0.423756767 | 0.857 | 0.783 | 1.25E−65 |
| Ephx2 | 2.03E−69 | −0.369303553 | 0.849 | 0.937 | 5.05E−65 |
| Alas1 | 4.49E−69 | −0.489837477 | 0.519 | 0.729 | 1.12E−64 |
| Apoa5 | 1.45E−67 | −0.298987336 | 0.924 | 0.974 | 3.60E−63 |
| Selenbp2 | 3.48E−67 | −0.383324562 | 0.868 | 0.937 | 8.65E−63 |
| Kng1 | 4.27E−67 | 0.265777676 | 0.987 | 0.991 | 1.06E−62 |
| Tram1 | 4.75E−67 | 0.411080257 | 0.7 | 0.512 | 1.18E−62 |
| Saa4 | 3.40E−66 | 0.375528565 | 0.823 | 0.749 | 8.44E−62 |
| C8b | 7.20E−66 | 0.318703124 | 0.898 | 0.881 | 1.79E−61 |
| mt-Nd6 | 1.13E−64 | −0.477851134 | 0.294 | 0.577 | 2.81E−60 |
| Ddt | 1.25E−64 | −0.422410948 | 0.64 | 0.829 | 3.10E−60 |
| Cyp2c37 | 2.32E−64 | −0.487522606 | 0.314 | 0.608 | 5.76E−60 |
| Ugt2b36 | 2.41E−64 | −0.329316239 | 0.875 | 0.93 | 5.98E−60 |
| Cyp2a5 | 2.49E−64 | −0.681077987 | 0.272 | 0.555 | 6.19E−60 |
| Serpina3n | 1.16E−63 | 0.413696164 | 0.719 | 0.574 | 2.88E−59 |
| Hpd | 7.70E−63 | −0.313324027 | 0.952 | 0.976 | 1.91E−58 |
| Inmt | 2.34E−62 | −0.44352952 | 0.831 | 0.884 | 5.81E−58 |
| Cox6a1 | 7.81E−62 | −0.366697192 | 0.746 | 0.887 | 1.94E−57 |
| Spcs3 | 8.20E−62 | 0.394206972 | 0.483 | 0.283 | 2.04E−57 |
| Serpina3c | 9.00E−61 | −0.408878947 | 0.198 | 0.385 | 2.23E−56 |
| Acsl1 | 1.58E−60 | −0.370316988 | 0.796 | 0.924 | 3.93E−56 |
| Qsox1 | 3.27E−60 | 0.342352868 | 0.784 | 0.707 | 8.13E−56 |
| Lrp1 | 4.30E−60 | 0.505128824 | 0.694 | 0.591 | 1.07E−55 |
| Chchd2 | 2.26E−59 | −0.394916772 | 0.801 | 0.916 | 5.60E−55 |
| Hsd3b7 | 2.44E−59 | −0.395256634 | 0.679 | 0.798 | 6.07E−55 |
| Ndufb11 | 4.81E−59 | −0.42752525 | 0.539 | 0.735 | 1.19E−54 |
| Sparcl1 | 8.16E−59 | −0.253616986 | 0 | 0.136 | 2.03E−54 |
| Cltc | 1.37E−58 | 0.438067775 | 0.61 | 0.414 | 3.40E−54 |
| Cyp2d10 | 4.66E−58 | −0.323151441 | 0.822 | 0.921 | 1.16E−53 |
| Sc5d | 6.09E−57 | 0.345016882 | 0.879 | 0.776 | 1.51E−52 |
| Gjb2 | 4.40E−56 | 0.391136188 | 0.709 | 0.572 | 1.09E−51 |
| Prg4 | 4.56E−56 | 0.430643187 | 0.421 | 0.188 | 1.13E−51 |
| Rdx | 5.22E−56 | 0.373557306 | 0.729 | 0.63 | 1.30E−51 |
| Fos | 6.68E−56 | 0.432003299 | 0.654 | 0.51 | 1.66E−51 |
| Rdh7 | 1.03E−55 | −0.269043262 | 0.932 | 0.969 | 2.55E−51 |
| Dcxr | 1.08E−55 | −0.410666503 | 0.436 | 0.631 | 2.69E−51 |
| C8a | 1.48E−55 | 0.333650228 | 0.902 | 0.887 | 3.67E−51 |
| Cpb2 | 2.27E−55 | 0.322179327 | 0.879 | 0.813 | 5.64E−51 |
| Akr1c6 | 3.55E−55 | −0.317638782 | 0.95 | 0.971 | 8.81E−51 |
| Fndc3b | 3.72E−55 | 0.348474602 | 0.291 | 0.096 | 9.25E−51 |
| Cat | 7.43E−55 | 0.262624418 | 0.978 | 0.971 | 1.84E−50 |
| Clpx | 9.40E−55 | −0.404735614 | 0.635 | 0.783 | 2.33E−50 |
| Uso1 | 9.63E−55 | 0.388069473 | 0.597 | 0.434 | 2.39E−50 |
| Kmo | 2.30E−54 | 0.302208311 | 0.778 | 0.732 | 5.72E−50 |
| Rpn1 | 3.25E−54 | 0.41242919 | 0.605 | 0.444 | 8.06E−50 |
| Hbb-bs | 4.64E−54 | 1.042581651 | 0.145 | 0.018 | 1.15E−49 |
| Tmed7 | 5.33E−54 | 0.386771014 | 0.563 | 0.367 | 1.32E−49 |
| Gm4076 | 1.10E−53 | −0.325469418 | 0.019 | 0.184 | 2.72E−49 |
| Pdia6 | 1.15E−53 | 0.336506977 | 0.779 | 0.712 | 2.86E−49 |
| Abca1 | 2.83E−53 | 0.439378423 | 0.556 | 0.377 | 7.04E−49 |
| Hspa13 | 4.18E−53 | 0.354844877 | 0.404 | 0.192 | 1.04E−48 |
| Cyb5r3 | 7.95E−53 | 0.339033056 | 0.8 | 0.725 | 1.97E−48 |
| Ubc | 9.16E−53 | −0.370487607 | 0.742 | 0.872 | 2.28E−48 |
| Apob | 3.60E−52 | 0.4272457 | 0.983 | 0.989 | 8.94E−48 |
| Pon1 | 5.27E−52 | −0.322748466 | 0.829 | 0.906 | 1.31E−47 |
| Slc7a2 | 9.89E−52 | 0.352564931 | 0.796 | 0.735 | 2.46E−47 |
| Saa1 | 1.82E−51 | 0.558211954 | 0.792 | 0.662 | 4.53E−47 |
| Nedd4 | 2.35E−51 | 0.377388355 | 0.647 | 0.524 | 5.84E−47 |
| Mug2 | 3.08E−51 | 0.370406564 | 0.784 | 0.738 | 7.64E−47 |
| AA474408 | 6.40E−51 | 0.43640813 | 0.278 | 0.094 | 1.59E−46 |
| Cyp2c70 | 7.75E−51 | 0.336400438 | 0.89 | 0.834 | 1.92E−46 |
| Cp | 1.59E−50 | 0.317404056 | 0.947 | 0.943 | 3.96E−46 |
| mt-Tq | 3.53E−50 | 0.287889985 | 0.225 | 0.057 | 8.77E−46 |
| Aadac | 4.09E−50 | −0.40376663 | 0.791 | 0.86 | 1.02E−45 |
| Usp9x | 1.24E−49 | 0.407617259 | 0.502 | 0.299 | 3.09E−45 |
| Orm2 | 1.38E−49 | 0.271460545 | 0.285 | 0.09 | 3.43E−45 |
| Igfbp2 | 4.52E−49 | −0.345087789 | 0.768 | 0.885 | 1.12E−44 |
| Hao1 | 7.16E−49 | 0.32303248 | 0.83 | 0.758 | 1.78E−44 |
| Oaz1 | 8.48E−49 | −0.374797984 | 0.594 | 0.75 | 2.11E−44 |
| Cps1 | 9.93E−49 | 0.32450205 | 0.982 | 0.971 | 2.47E−44 |
| Retsat | 1.08E−48 | −0.365840188 | 0.709 | 0.83 | 2.69E−44 |
| Gm5096 | 1.55E−48 | −0.363089147 | 0.287 | 0.468 | 3.85E−44 |
| Gchfr | 3.21E−48 | −0.386867237 | 0.604 | 0.768 | 7.98E−44 |
| Mup8 | 5.50E−48 | −0.613334367 | 0.218 | 0.373 | 1.37E−43 |
| Atp8b1 | 6.17E−48 | 0.362807422 | 0.391 | 0.182 | 1.53E−43 |
| Zfp683 | 1.06E−47 | −0.421395009 | 0.001 | 0.116 | 2.64E−43 |
| Tmem30a | 1.20E−47 | 0.332760453 | 0.766 | 0.664 | 2.98E−43 |
| Cxcl1 | 1.29E−47 | 0.449766784 | 0.482 | 0.314 | 3.20E−43 |
| Pten | 1.30E−46 | 0.355902258 | 0.612 | 0.458 | 3.22E−42 |
| mmu-mir-6236 | 1.50E−46 | 0.787474192 | 0.896 | 0.837 | 3.73E−42 |
| Hspa8 | 1.60E−46 | −0.359978165 | 0.58 | 0.756 | 3.96E−42 |
| Hs3st3b1 | 2.33E−46 | 0.343007923 | 0.442 | 0.241 | 5.78E−42 |
| Prkd3 | 2.71E−46 | 0.366517526 | 0.631 | 0.49 | 6.72E−42 |
| Dnajc12 | 4.61E−46 | 0.322094554 | 0.275 | 0.108 | 1.15E−41 |
| Etnppl | 6.89E−46 | −0.428291487 | 0.409 | 0.606 | 1.71E−41 |
| Rsp14 | 9.86E−46 | −0.292251029 | 0.815 | 0.917 | 2.45E−41 |
| Hint2 | 1.17E−45 | −0.37016853 | 0.51 | 0.676 | 2.90E−41 |
| Mup20 | 1.28E−45 | −0.281871664 | 0.988 | 1 | 3.18E−41 |
| Snord118 | 3.42E−45 | 0.391412496 | 0.449 | 0.248 | 8.50E−41 |
| Csde1 | 3.72E−45 | 0.326883793 | 0.687 | 0.577 | 9.23E−41 |
| Adh4 | 1.11E−44 | 0.376928101 | 0.667 | 0.521 | 2.75E−40 |
| Rps2 | 1.35E−44 | −0.384454173 | 0.583 | 0.757 | 3.34E−40 |
| Lpgat1 | 1.38E−44 | 0.394625955 | 0.635 | 0.502 | 3.42E−40 |
| S100a1 | 1.97E−44 | −0.376186192 | 0.494 | 0.661 | 4.89E−40 |
| Acot1 | 2.09E−44 | −0.286204152 | 0.059 | 0.237 | 5.19E−40 |
| mt-Tm | 2.31E−44 | 0.284684642 | 0.274 | 0.093 | 5.75E−40 |
| Il13ra1 | 4.07E−44 | 0.30296595 | 0.33 | 0.143 | 1.01E−39 |
| Rab1 | 8.74E−44 | 0.352627435 | 0.588 | 0.438 | 2.17E−39 |
| Rrbp1 | 1.08E−43 | 0.306944991 | 0.823 | 0.768 | 2.68E−39 |
| Khk | 1.45E−43 | −0.29761703 | 0.822 | 0.863 | 3.60E−39 |
| Fabp2 | 1.67E−43 | −0.39219613 | 0.437 | 0.622 | 4.14E−39 |
| Efna1 | 2.79E−43 | 0.336097237 | 0.482 | 0.309 | 6.92E−39 |
| Sds | 5.49E−43 | −0.407006722 | 0.443 | 0.67 | 1.36E−38 |
| Ak3 | 8.39E−43 | 0.289040998 | 0.772 | 0.7 | 2.08E−38 |
| Slc38a4 | 1.50E−42 | 0.263698621 | 0.897 | 0.877 | 3.73E−38 |
| Slc3a1 | 1.55E−42 | 0.340997565 | 0.378 | 0.189 | 3.84E−38 |
| Cmah | 1.72E−42 | 0.361552447 | 0.607 | 0.465 | 4.27E−38 |
| Sar1b | 3.22E−42 | −0.317607513 | 0.648 | 0.772 | 8.00E−38 |
| Itih2 | 3.32E−42 | 0.298659728 | 0.922 | 0.884 | 8.24E−38 |
| Acox1 | 3.87E−42 | 0.275171428 | 0.966 | 0.966 | 9.60E−38 |
| Plg | 4.24E−42 | 0.261736006 | 0.975 | 0.961 | 1.05E−37 |
| Eef2 | 1.35E−41 | 0.270115373 | 0.89 | 0.873 | 3.35E−37 |
| Trp53inp1 | 1.49E−41 | 0.367960906 | 0.473 | 0.308 | 3.70E−37 |
| Cox7a2 | 1.23E−40 | −0.363458039 | 0.638 | 0.784 | 3.06E−36 |
| mt-Atp6 | 1.56E−40 | −0.573743033 | 0.289 | 0.452 | 3.87E−36 |
| Cfi | 3.01E−40 | 0.251606433 | 0.914 | 0.905 | 7.48E−36 |
| Eif4g1 | 4.16E−40 | 0.310782755 | 0.745 | 0.669 | 1.03E−35 |
| Prdx1 | 5.51E−40 | −0.267665106 | 0.839 | 0.927 | 1.37E−35 |
| Atxn1 | 1.00E−39 | 0.304881734 | 0.311 | 0.137 | 2.49E−35 |
| Ssr1 | 2.80E−39 | 0.327818552 | 0.523 | 0.339 | 6.95E−35 |
| Etnk1 | 3.37E−39 | 0.352068809 | 0.49 | 0.328 | 8.38E−35 |
| Ugt2a3 | 4.67E−39 | −0.333441405 | 0.641 | 0.773 | 1.16E−34 |
| Dstn | 1.19E−38 | 0.324301394 | 0.57 | 0.37 | 2.95E−34 |
| Mpc1 | 1.91E−38 | −0.281762702 | 0.184 | 0.369 | 4.73E−34 |
| Cyp2c54 | 2.08E−38 | −0.326859698 | 0.566 | 0.759 | 5.16E−34 |
| Nars | 2.39E−38 | 0.353230364 | 0.593 | 0.471 | 5.94E−34 |
| Gas5 | 3.41E−38 | −0.336153154 | 0.337 | 0.527 | 8.46E−34 |
| Ugt2b1 | 4.76E−38 | −0.289461704 | 0.761 | 0.871 | 1.18E−33 |
| Serp1 | 7.07E−38 | 0.312399212 | 0.809 | 0.721 | 1.76E−33 |
| Mtus1 | 1.54E−37 | 0.362060296 | 0.563 | 0.42 | 3.83E−33 |
| Cox5b | 1.54E−37 | −0.337825031 | 0.541 | 0.726 | 3.83E−33 |
| Fdx1 | 1.76E−37 | −0.344715083 | 0.482 | 0.625 | 4.37E−33 |
| Igfbp1 | 2.33E−37 | −0.316195447 | 0.519 | 0.717 | 5.79E−33 |
| Ibtk | 2.91E−37 | 0.331106132 | 0.43 | 0.247 | 7.23E−33 |
| Fxyd1 | 3.75E−37 | −0.311759144 | 0.547 | 0.721 | 9.32E−33 |
| Slc41a2 | 3.84E−37 | 0.278262888 | 0.302 | 0.134 | 9.55E−33 |
| Nab1 | 7.05E−37 | 0.33421071 | 0.491 | 0.315 | 1.75E−32 |
| Hook1 | 8.56E−37 | 0.327423989 | 0.457 | 0.291 | 2.13E−32 |
| Stard4 | 9.03E−37 | 0.351527506 | 0.559 | 0.399 | 2.24E−32 |
| Usp4 | 9.90E−37 | 0.324732042 | 0.476 | 0.286 | 2.46E−32 |
| Ndufb4 | 2.36E−36 | −0.325145539 | 0.309 | 0.49 | 5.86E−32 |
| Cml1 | 2.38E−36 | −0.326909468 | 0.628 | 0.757 | 5.91E−32 |
| Ndufs6 | 2.53E−36 | −0.359160953 | 0.563 | 0.724 | 6.27E−32 |
| Sec61a1 | 2.54E−36 | 0.313944633 | 0.607 | 0.476 | 6.31E−32 |
| Arcn1 | 2.88E−36 | 0.327775216 | 0.53 | 0.366 | 7.15E−32 |
| Nr5a2 | 3.30E−36 | 0.322039014 | 0.403 | 0.228 | 8.21E−32 |
| Cyp2c50 | 3.34E−36 | −0.290483498 | 0.739 | 0.888 | 8.29E−32 |
| Aldh3a2 | 4.28E−36 | −0.300776093 | 0.306 | 0.529 | 1.06E−31 |
| BC025446 | 4.71E−36 | 0.29422883 | 0.486 | 0.306 | 1.17E−31 |
| Cml2 | 4.99E−36 | 0.265441744 | 0.896 | 0.857 | 1.24E−31 |
| Il6st | 6.36E−36 | 0.333486533 | 0.448 | 0.273 | 1.58E−31 |
| Ndufb2 | 9.30E−36 | −0.341226284 | 0.468 | 0.629 | 2.31E−31 |
| Lman1 | 9.78E−36 | 0.29860668 | 0.75 | 0.655 | 2.43E−31 |
| Lrrc59 | 1.50E−35 | 0.318083941 | 0.449 | 0.28 | 3.72E−31 |
| Mlec | 2.35E−35 | 0.324558531 | 0.496 | 0.323 | 5.82E−31 |
| Copa | 3.03E−35 | 0.345094339 | 0.525 | 0.363 | 7.52E−31 |
| Ipo7 | 4.40E−35 | 0.25915239 | 0.351 | 0.168 | 1.09E−30 |
| Calu | 4.76E−35 | 0.304112196 | 0.407 | 0.236 | 1.18E−30 |
| Aox3 | 5.13E−35 | 0.287845433 | 0.916 | 0.894 | 1.27E−30 |
| Acsl4 | 5.53E−35 | 0.314743242 | 0.478 | 0.323 | 1.37E−30 |
| Sel1l | 8.70E−35 | 0.33595882 | 0.544 | 0.392 | 2.16E−30 |
| Ctsc | 1.04E−34 | 0.300373343 | 0.336 | 0.271 | 2.58E−30 |
| Cmbl | 1.15E−34 | −0.312422041 | 0.523 | 0.678 | 2.85E−30 |
| Rps15 | 1.42E−34 | −0.356137747 | 0.379 | 0.528 | 3.53E−30 |
| Cald1 | 1.88E−34 | 0.258910981 | 0.79 | 0.749 | 4.66E−30 |
| Atp5e | 3.07E−34 | −0.313641686 | 0.688 | 0.832 | 7.62E−30 |
| Adck3 | 4.29E−34 | −0.28251534 | 0.68 | 0.764 | 1.06E−29 |
| Cyp4a14 | 4.45E−34 | −0.372863761 | 0.245 | 0.431 | 1.10E−29 |
| Cfhr2 | 5.63E−34 | −0.321928662 | 0.814 | 0.868 | 1.40E−29 |
| Ndufb6 | 6.93E−34 | −0.306403298 | 0.636 | 0.759 | 1.72E−29 |
| Uggt1 | 9.21E−34 | 0.284457726 | 0.385 | 0.204 | 2.29E−29 |
| Rarres2 | 1.49E−33 | −0.266616471 | 0.83 | 0.905 | 3.70E−29 |
| Srpr | 2.41E−33 | 0.305179041 | 0.532 | 0.387 | 5.97E−29 |
| Id3 | 2.57E−33 | −0.351088732 | 0.278 | 0.426 | 6.37E−29 |
| Ndufa6 | 2.87E−33 | −0.306815302 | 0.692 | 0.798 | 7.12E−29 |
| Nop10 | 3.01E−33 | −0.264928395 | 0.227 | 0.394 | 7.48E−29 |
| Tob1 | 4.96E−33 | 0.312925608 | 0.565 | 0.43 | 1.23E−28 |
| Nrp1 | 5.28E−33 | 0.334097753 | 0.426 | 0.253 | 1.31E−28 |
| Vkorc1 | 5.36E−33 | −0.297366597 | 0.38 | 0.54 | 1.33E−28 |
| Stt3b | 6.49E−33 | 0.319405267 | 0.485 | 0.335 | 1.61E−28 |
| Trip12 | 6.80E−33 | 0.328304235 | 0.504 | 0.345 | 1.69E−28 |
| Dnajb11 | 1.79E−32 | 0.315357308 | 0.557 | 0.43 | 4.44E−28 |
| Gstp1 | 1.86E−32 | −0.430197368 | 0.743 | 0.822 | 4.63E−28 |
| Atp5f1 | 2.05E−32 | −0.28724025 | 0.8 | 0.869 | 5.09E−28 |
| Prdx4 | 3.84E−32 | −0.307442113 | 0.47 | 0.607 | 9.54E−28 |
| Ces3b | 4.12E−32 | −0.286685415 | 0.683 | 0.787 | 1.02E−27 |
| Bzw1 | 4.22E−32 | 0.284147153 | 0.487 | 0.344 | 1.05E−27 |
| Srp72 | 1.97E−31 | 0.310685136 | 0.597 | 0.461 | 4.90E−27 |
| Ptprf | 4.48E−31 | 0.307126385 | 0.588 | 0.464 | 1.11E−26 |
| Ell2 | 4.71E−31 | 0.288090516 | 0.552 | 0.416 | 1.17E−26 |
| Atp13a3 | 6.78E−31 | 0.303730845 | 0.546 | 0.41 | 1.68E−26 |
| Ndufc2 | 7.73E−31 | −0.308039389 | 0.465 | 0.579 | 1.92E−26 |
| Pnpla7 | 8.71E−31 | 0.29608325 | 0.678 | 0.572 | 2.16E−26 |
| Sco2 | 1.20E−30 | −0.292870629 | 0.346 | 0.508 | 2.98E−26 |
| Edem1 | 1.31E−30 | 0.286881059 | 0.566 | 0.429 | 3.26E−26 |
| Akr1c19 | 1.49E−30 | −0.272667086 | 0.389 | 0.527 | 3.70E−26 |
| Slc39a14 | 1.88E−30 | 0.323040019 | 0.495 | 0.343 | 4.66E−26 |
| Uqcr11 | 2.43E−30 | −0.263241396 | 0.769 | 0.879 | 6.03E−26 |
| Met | 2.91E−30 | 0.288332662 | 0.434 | 0.274 | 7.22E−26 |
| Man2a1 | 3.00E−30 | 0.28327895 | 0.675 | 0.583 | 7.46E−26 |
| Lgr4 | 3.03E−30 | 0.268964381 | 0.399 | 0.226 | 7.52E−26 |
| Rpl13 | 1.27E−29 | −0.299693639 | 0.306 | 0.464 | 3.17E−25 |
| Osbp19 | 1.49E−29 | 0.278773264 | 0.569 | 0.448 | 3.69E−25 |
| Ctnnb1 | 2.29E−29 | 0.295681078 | 0.566 | 0.436 | 5.67E−25 |
| Slc38a10 | 2.50E−29 | 0.285548034 | 0.449 | 0.291 | 6.20E−25 |
| Arhgef12 | 2.67E−29 | 0.304679431 | 0.541 | 0.408 | 6.64E−25 |
| Mtss1 | 2.84E−29 | 0.287581972 | 0.581 | 0.454 | 7.06E−25 |
| Pik3c2a | 9.21E−29 | 0.25938652 | 0.36 | 0.198 | 2.29E−24 |
| Acly | 1.12E−28 | 0.328803101 | 0.597 | 0.445 | 2.79E−24 |
| Sc4mol | 2.18E−28 | −0.273479653 | 0.5 | 0.642 | 5.42E−24 |
| Rplp1 | 2.38E−28 | −0.286640389 | 0.597 | 0.757 | 5.92E−24 |
| Arhgap5 | 2.81E−28 | 0.295235575 | 0.566 | 0.454 | 6.98E−24 |
| Prdx5 | 2.81E−28 | −0.260937182 | 0.731 | 0.834 | 6.99E−24 |
| Zbtb20 | 4.95E−28 | 0.379090332 | 0.566 | 0.445 | 1.23E−23 |
| Eprs | 5.12E−28 | 0.301270543 | 0.587 | 0.488 | 1.27E−23 |
| H6pd | 5.18E−28 | 0.261357904 | 0.675 | 0.595 | 1.29E−23 |
| Narf | 7.05E−28 | 0.262933124 | 0.411 | 0.253 | 1.75E−23 |
| Trib1 | 9.72E−28 | 0.287458438 | 0.455 | 0.308 | 2.41E−23 |
| Esd | 1.31E−27 | −0.25379488 | 0.67 | 0.784 | 3.25E−23 |
| Atp6v1f | 1.33E−27 | −0.252893521 | 0.244 | 0.403 | 3.29E−23 |
| Grlf1 | 1.48E−27 | 0.295046691 | 0.413 | 0.257 | 3.69E−23 |
| Bnip3 | 1.50E−27 | −0.292803703 | 0.39 | 0.55 | 3.73E−23 |
| Osbpl1a | 1.54E−27 | 0.272788799 | 0.592 | 0.473 | 3.82E−23 |
| Rps9 | 1.67E−27 | −0.313951151 | 0.519 | 0.675 | 4.15E−23 |
| Erp44 | 1.71E−27 | 0.279450299 | 0.469 | 0.336 | 4.24E−23 |
| Tomm70a | 1.75E−27 | 0.268786029 | 0.6 | 0.494 | 4.36E−23 |
| Txndc5 | 1.86E−27 | 0.290124872 | 0.48 | 0.327 | 4.61E−23 |
| Iqgap2 | 1.95E−27 | 0.254698841 | 0.748 | 0.677 | 4.85E−23 |
| Arl5a | 2.07E−27 | 0.264805346 | 0.459 | 0.289 | 5.13E−23 |
| Cox7b | 4.18E−27 | −0.267397469 | 0.768 | 0.878 | 1.04E−22 |
| Selenbp1 | 4.28E−27 | −0.280021704 | 0.563 | 0.699 | 1.06E−22 |
| Ftl1 | 4.31E−27 | −0.349777423 | 0.27 | 0.409 | 1.07E−22 |
| Lpin2 | 6.30E−27 | 0.277576437 | 0.7 | 0.597 | 1.56E−22 |
| Magt1 | 6.93E−27 | 0.263295188 | 0.52 | 0.412 | 1.72E−22 |
| Gm24245 | 1.17E−26 | 0.42420314 | 0.369 | 0.214 | 2.90E−22 |
| Caprin1 | 1.32E−26 | 0.262825966 | 0.567 | 0.481 | 3.29E−22 |
| Azin1 | 1.45E−26 | 0.270139699 | 0.46 | 0.322 | 3.60E−22 |
| Fam120a | 1.66E−26 | 0.280043634 | 0.51 | 0.391 | 4.13E−22 |
| Atp11c | 1.81E−26 | 0.260536506 | 0.649 | 0.587 | 4.49E−22 |
| Surf4 | 2.20E−26 | 0.258577589 | 0.656 | 0.551 | 5.46E−22 |
| Leap2 | 2.26E−26 | −0.274778129 | 0.469 | 0.632 | 5.61E−22 |
| Plin5 | 3.28E−26 | −0.277759837 | 0.181 | 0.315 | 8.16E−22 |
| Hspe1 | 3.41E−26 | −0.290582957 | 0.413 | 0.557 | 8.48E−22 |
| Copb2 | 3.61E−26 | 0.283928956 | 0.595 | 0.471 | 8.98E−22 |
| Gm20594 | 4.38E−26 | −0.360000649 | 0.204 | 0.294 | 1.09E−21 |
| BC089597 | 1.29E−25 | 0.262237515 | 0.593 | 0.488 | 3.21E−21 |
| Tmem243 | 1.37E−25 | −0.26555147 | 0.407 | 0.557 | 3.41E−21 |
| Eci1 | 1.76E−25 | −0.276383144 | 0.581 | 0.709 | 4.37E−21 |
| Cpne3 | 1.93E−25 | 0.258007443 | 0.456 | 0.31 | 4.78E−21 |
| Cideb | 1.94E−25 | −0.27071201 | 0.557 | 0.676 | 4.82E−21 |
| Gch1 | 2.96E−25 | −0.282499843 | 0.502 | 0.643 | 7.35E−21 |
| Dnajb9 | 3.65E−25 | 0.25864192 | 0.373 | 0.241 | 9.07E−21 |
| Dennd5b | 3.78E−25 | 0.293458876 | 0.476 | 0.334 | 9.38E−21 |
| Srebf1 | 5.27E−25 | 0.27619787 | 0.439 | 0.307 | 1.31E−20 |
| Ide | 5.87E−25 | 0.251691529 | 0.604 | 0.494 | 1.46E−20 |
| Kpnb1 | 9.41E−25 | 0.280415627 | 0.447 | 0.311 | 2.34E−20 |
| Nr1i3 | 1.39E−24 | −0.282083592 | 0.237 | 0.377 | 3.46E−20 |
| Xiap | 1.94E−24 | 0.26016306 | 0.504 | 0.378 | 4.81E−20 |
| Nadk2 | 3.06E−24 | 0.257852312 | 0.539 | 0.417 | 7.60E−20 |
| Proz | 3.97E−24 | 0.263204472 | 0.622 | 0.53 | 9.85E−20 |
| Prkaa2 | 4.37E−24 | 0.263640766 | 0.451 | 0.315 | 1.09E−19 |
| Stat3 | 4.97E−24 | 0.297428438 | 0.459 | 0.318 | 1.23E−19 |
| Ostc | 5.87E−24 | 0.265829876 | 0.514 | 0.357 | 1.46E−19 |
| Ireb2 | 6.99E−24 | 0.265741209 | 0.469 | 0.321 | 1.74E−19 |
| Mt1 | 7.07E−24 | −0.348193009 | 0.41 | 0.556 | 1.76E−19 |
| Sec31a | 7.08E−24 | 0.263042758 | 0.462 | 0.31 | 1.76E−19 |
| Brd2 | 2.14E−23 | 0.266388486 | 0.512 | 0.408 | 5.30E−19 |
| Fcgrt | 2.28E−23 | −0.269314211 | 0.461 | 0.599 | 5.65E−19 |
| Gpam | 6.56E−23 | 0.250377871 | 0.329 | 0.199 | 1.63E−18 |
| Cyp27a1 | 9.47E−23 | −0.251578373 | 0.759 | 0.837 | 2.35E−18 |
| Erbb2ip | 1.42E−22 | 0.279650574 | 0.482 | 0.364 | 3.54E−18 |
| Ranbp2 | 2.50E−22 | 0.26594209 | 0.484 | 0.338 | 6.20E−18 |
| Raf13 | 3.68E−22 | 0.255296715 | 0.612 | 0.516 | 9.13E−18 |
| Txnrd1 | 5.19E−22 | 0.260866185 | 0.564 | 0.441 | 1.29E−17 |
| Mbnl1 | 6.12E−22 | 0.25020478 | 0.428 | 0.297 | 1.52E−17 |
| Jak1 | 8.54E−22 | 0.254122657 | 0.482 | 0.342 | 2.12E−17 |
| Psme4 | 9.89E−22 | 0.262584214 | 0.58 | 0.478 | 2.46E−17 |
| Golga4 | 2.75E−21 | 0.271769698 | 0.447 | 0.31 | 6.83E−17 |
| Ehhadh | 6.02E−21 | −0.250472633 | 0.552 | 0.691 | 1.49E−16 |
| Lasp1 | 7.14E−21 | 0.253021439 | 0.361 | 0.243 | 1.77E−16 |
| Rpl32 | 9.18E−21 | −0.254910119 | 0.636 | 0.757 | 2.28E−16 |
| Copz1 | 1.57E−20 | 0.257722711 | 0.501 | 0.385 | 3.89E−16 |
| Eif4b | 2.82E−20 | 0.255987641 | 0.542 | 0.438 | 6.99E−16 |
| Gm23935 | 3.29E−20 | 0.721300311 | 0.919 | 0.925 | 8.16E−16 |
| Ddost | 4.03E−20 | 0.250356033 | 0.493 | 0.392 | 1.00E−15 |
| Fasn | 5.17E−20 | 0.291406736 | 0.449 | 0.345 | 1.28E−15 |
| Sh3bgrl | 5.20E−20 | 0.25128227 | 0.512 | 0.385 | 1.29E−15 |
| Macf1 | 1.18E−19 | 0.261666188 | 0.349 | 0.219 | 2.94E−15 |
| Deptor | 2.26E−19 | 0.260618372 | 0.583 | 0.473 | 5.60E−15 |
| Prox1 | 2.48E−19 | 0.266329524 | 0.477 | 0.351 | 6.16E−15 |
| Sbno1 | 2.55E−19 | 0.250529348 | 0.36 | 0.228 | 6.33E−15 |
| Atp1a1 | 5.55E−19 | 0.270031448 | 0.532 | 0.457 | 1.38E−14 |
| Ubb | 1.44E−17 | −0.259339434 | 0.329 | 0.45 | 3.57E−13 |
| Gstm3 | 4.48E−16 | −0.318590137 | 0.293 | 0.414 | 1.11E−11 |
| Por | 3.09E−13 | −0.271635533 | 0.589 | 0.675 | 7.68E−09 |
| Mup2 | 1.04E−11 | −0.274243623 | 0.364 | 0.392 | 2.59E−07 |
| Mup1 | 0.000864034 | −0.261837735 | 0.588 | 0.583 | 1 |

TABLE 4a
Differentially expressed genes between time point A48 and untreated (UT)

| | p_val | avg_logFC | pct.1 | pct.2 | p_val_adj |
|---|---|---|---|---|---|
| Cyp4a14 | 0 | 1.78777169 | 0.991 | 0.431 | 0 |
| Cyp4a10 | 0 | 1.14855488 | 0.991 | 0.657 | 0 |
| Mat1a | 0 | 1.05694824 | 1 | 0.966 | 0 |
| Hmgcs2 | 0 | 0.782364 | 1 | 0.986 | 0 |
| Mup11 | 0 | −1.6799694 | 0.896 | 1 | 0 |
| Mup18 | 0 | −1.9532293 | 0.887 | 0.997 | 0 |
| Mup6 | 0 | −2.0417075 | 0.885 | 0.992 | 0 |
| Mup9 | 0 | −2.0539847 | 0.589 | 0.976 | 0 |
| Mup17 | 0 | −2.3251338 | 0.962 | 1 | 0 |
| Mup19 | 0 | −2.5024654 | 0.938 | 1 | 0 |
| mt-Nd2 | 5.70E−292 | −1.0887684 | 0.995 | 0.999 | 1.42E−287 |
| mt-Co1 | 1.02E−291 | −1.2589436 | 0.995 | 0.999 | 2.52E−287 |
| Mfsd2a | 3.98E−286 | 0.78121324 | 0.731 | 0.148 | 9.87E−282 |
| Angpt13 | 1.31E−281 | 0.7223486 | 0.995 | 0.944 | 3.25E−277 |
| Apoc2 | 2.70E−279 | 0.96935076 | 0.98 | 0.802 | 6.70E−275 |
| Ces1c | 2.09E−273 | 0.55433171 | 0.999 | 0.985 | 5.20E−269 |
| Selenbp2 | 4.62E−273 | −1.1339606 | 0.807 | 0.937 | 1.15E−268 |
| Chchd10 | 4.58E−272 | 0.81121599 | 0.995 | 0.944 | 1.14E−267 |
| Mup7 | 5.30E−265 | −1.0899502 | 0.93 | 0.998 | 1.32E−260 |
| Apoe | 1.09E−264 | 0.47353426 | 1 | 1 | 2.72E−260 |
| mt-Nd4 | 3.35E−263 | −1.0213393 | 0.997 | 0.998 | 8.31E−259 |
| mt-Cytb | 1.81E−254 | −1.0028016 | 0.996 | 0.999 | 4.51E−250 |
| mt-Rnr2 | 3.30E−250 | −0.8153022 | 1 | 1 | 8.18E−246 |
| mt-Nd5 | 1.44E−247 | −1.0547847 | 0.988 | 0.995 | 3.56E−243 |
| Retsat | 6.60E−238 | 0.79666675 | 0.988 | 0.83 | 1.64E−233 |
| Mup16 | 2.03E−236 | −1.084786 | 0.715 | 0.934 | 5.04E−232 |
| Apoh | 2.06E−234 | 0.46628252 | 0.998 | 0.986 | 5.13E−230 |
| Mup21 | 2.18E−228 | −1.0729318 | 0.777 | 0.929 | 5.42E−224 |
| Ahsg | 1.32E−226 | 0.36968503 | 1 | 1 | 3.27E−222 |
| Abcd2 | 1.26E−222 | 0.32535888 | 0.473 | 0.052 | 3.13E−218 |
| Aldob | 4.71E−217 | 0.49713861 | 1 | 0.996 | 1.17E−212 |
| Rgs16 | 1.46E−216 | 0.48956756 | 0.601 | 0.113 | 3.62E−212 |
| Ttc36 | 4.07E−214 | 0.52379567 | 0.996 | 0.95 | 1.01E−209 |
| Dbp | 1.96E−213 | 0.28461585 | 0.52 | 0.07 | 4.86E−209 |
| Slco1a4 | 4.08E−213 | 0.62444908 | 0.77 | 0.238 | 1.01E−208 |
| Agt | 4.67E−213 | 0.63305205 | 0.981 | 0.817 | 1.16E−208 |
| Herpud1 | 7.00E−209 | 0.65043654 | 0.985 | 0.803 | 1.74E−204 |
| Hrg | 1.97E−205 | 0.60700462 | 0.987 | 0.831 | 4.88E−201 |
| Gc | 4.59E−200 | 0.34083171 | 1 | 0.999 | 1.14E−195 |
| Cp | 4.65E−198 | 0.55546665 | 0.995 | 0.943 | 1.15E−193 |
| Slc25a47 | 3.79E−196 | 0.66741013 | 0.992 | 0.927 | 9.42E−192 |
| mt-Nd1 | 3.99E−196 | −0.5982047 | 1 | 1 | 9.90E−192 |
| Vtn | 1.22E−195 | 0.50147723 | 1 | 0.983 | 3.02E−191 |
| F2 | 3.37E−193 | 0.42280245 | 0.999 | 0.969 | 8.36E−189 |
| Gm13775 | 1.59E−192 | −1.1035721 | 0.321 | 0.761 | 3.94E−188 |
| Snord118 | 1.79E−192 | 0.65098072 | 0.766 | 0.248 | 4.45E−188 |
| mt-Rnr1 | 1.01E−191 | −0.6354582 | 1 | 1 | 2.51E−187 |
| Reep6 | 1.19E−191 | 0.66052002 | 0.967 | 0.794 | 2.95E−187 |
| Tef | 1.43E−190 | 0.37649582 | 0.651 | 0.148 | 3.55E−186 |
| Ttpa | 3.04E−190 | 0.57816408 | 0.991 | 0.869 | 7.56E−186 |
| St3gal5 | 7.03E−187 | 0.66332104 | 0.914 | 0.49 | 1.75E−182 |
| Sepp1 | 2.92E−186 | 0.38687237 | 1 | 1 | 7.25E−182 |
| Serping1 | 1.89E−182 | 0.5901636 | 0.987 | 0.87 | 4.70E−178 |
| Decr1 | 2.76E−180 | 0.57780557 | 0.96 | 0.694 | 6.84E−176 |
| Gm23935 | 2.81E−179 | 0.70920114 | 0.997 | 0.925 | 6.98E−175 |
| Ndrg2 | 9.21E−178 | 0.43980324 | 1 | 0.939 | 2.29E−173 |
| Sult1a1 | 8.15E−177 | 0.59346826 | 0.963 | 0.707 | 2.02E−172 |
| Mup4 | 1.14E−176 | −1.2590554 | 0.78 | 0.898 | 2.84E−172 |
| Apoa5 | 1.06E−175 | 0.42466196 | 0.998 | 0.974 | 2.63E−171 |
| Mup5 | 8.55E−175 | −1.4352143 | 0.655 | 0.839 | 2.12E−170 |
| Rbp4 | 2.98E−170 | 0.38026101 | 1 | 0.999 | 7.41E−166 |
| Rdh7 | 2.87E−169 | 0.4431568 | 0.996 | 0.969 | 7.14E−165 |
| Serpina3m | 7.11E−164 | 0.58543579 | 0.949 | 0.722 | 1.77E−159 |
| Nudt7 | 1.70E−162 | −1.1432695 | 0.738 | 0.83 | 4.22E−158 |
| Fth1 | 1.20E−161 | 0.42544729 | 0.996 | 0.978 | 2.98E−157 |
| Gm6484 | 1.57E−161 | 0.43191488 | 0.627 | 0.174 | 3.91E−157 |
| Ttr | 4.92E−161 | 0.42565826 | 1 | 1 | 1.22E−156 |
| Grn | 2.23E−159 | 0.55458402 | 0.844 | 0.396 | 5.54E−155 |
| Pabpc1 | 2.53E−159 | 0.59469924 | 0.912 | 0.592 | 6.28E−155 |
| Fkbp5 | 2.41E−156 | 0.31305366 | 0.54 | 0.117 | 5.98E−152 |
| Cdo1 | 4.87E−154 | 0.46952727 | 0.997 | 0.96 | 1.21E−149 |
| Lcat | 2.30E−152 | 0.52223232 | 0.973 | 0.781 | 5.72E−148 |
| Car3 | 2.49E−152 | −0.6790672 | 0.957 | 0.974 | 6.19E−148 |
| Syvn1 | 2.49E−150 | 0.34196184 | 0.603 | 0.165 | 6.18E−146 |
| Oaf | 6.13E−146 | 0.52764092 | 0.969 | 0.794 | 1.52E−141 |
| Plin5 | 4.86E−145 | 0.42261205 | 0.788 | 0.315 | 1.21E−140 |
| Cyp17a1 | 2.78E−143 | 0.25634278 | 0.338 | 0.044 | 6.90E−139 |
| Mup15 | 3.34E−143 | −1.2816696 | 0.209 | 0.617 | 8.29E−139 |
| Apoa4 | 7.77E−143 | 0.74347161 | 0.778 | 0.339 | 1.93E−138 |
| P4hb | 1.09E−142 | 0.38448979 | 0.995 | 0.941 | 2.70E−138 |
| Igfbp4 | 6.65E−142 | 0.4496666 | 0.99 | 0.866 | 1.65E−137 |
| Ambp | 7.17E−142 | 0.31533589 | 1 | 0.993 | 1.78E−137 |
| Elovl3 | 1.16E−140 | −0.8184539 | 0.669 | 0.827 | 2.87E−136 |
| Rpl38 | 2.08E−140 | 0.32217952 | 0.596 | 0.16 | 5.16E−136 |
| Hmgcl | 8.90E−140 | 0.57017343 | 0.956 | 0.729 | 2.21E−135 |
| Abcb4 | 1.10E−139 | 0.51123675 | 0.956 | 0.675 | 2.74E−135 |
| Hp | 4.14E−136 | 0.30554085 | 1 | 0.999 | 1.03E−131 |
| Ildr2 | 4.96E−136 | 0.28219186 | 0.588 | 0.159 | 1.23E−131 |
| Ube2r2 | 3.18E−135 | 0.50268344 | 0.884 | 0.525 | 7.90E−131 |
| Bfar | 7.09E−135 | 0.34808175 | 0.742 | 0.27 | 1.76E−130 |
| Cyp2a12 | 3.08E−133 | 0.49599281 | 0.981 | 0.884 | 7.65E−129 |
| Wbp1l | 7.93E−131 | 0.51781333 | 0.91 | 0.606 | 1.97E−126 |
| Hsd17b13 | 2.60E−130 | 0.74833751 | 0.934 | 0.74 | 6.45E−126 |
| Slc35g1 | 4.66E−130 | 0.25441963 | 0.521 | 0.133 | 1.16E−125 |
| Serpina3n | 4.85E−130 | 0.51010138 | 0.902 | 0.574 | 1.20E−125 |
| Mup3 | 7.70E−130 | −0.3534779 | 1 | 1 | 1.91E−125 |
| Apoc3 | 2.95E−129 | 0.35021276 | 1 | 0.993 | 7.33E−125 |
| mt-Co2 | 8.06E−129 | −0.8270395 | 0.14 | 0.629 | 2.00E−124 |
| Prodh2 | 2.15E−128 | 0.49365697 | 0.918 | 0.611 | 5.34E−124 |
| Aldh9a1 | 3.44E−128 | 0.46052647 | 0.964 | 0.763 | 8.53E−124 |
| F10 | 1.60E−127 | 0.41275991 | 0.989 | 0.895 | 3.97E−123 |
| Tmbim6 | 2.08E−127 | 0.40188954 | 0.996 | 0.942 | 5.16E−123 |
| Ces1b | 2.43E−126 | 0.49918312 | 0.838 | 0.437 | 6.03E−122 |
| Akr1c6 | 2.52E−126 | −0.5780467 | 0.955 | 0.971 | 6.27E−122 |
| Lpin1 | 2.69E−126 | 0.52324678 | 0.736 | 0.31 | 6.67E−122 |
| Sh3glb1 | 5.75E−126 | 0.38610114 | 0.695 | 0.258 | 1.43E−121 |
| Slc27a5 | 6.41E−125 | 0.40331028 | 0.987 | 0.893 | 1.59E−120 |
| Vnn3 | 8.41E−125 | 0.33728247 | 0.684 | 0.241 | 2.09E−120 |
| Pnpla2 | 2.09E−123 | 0.3417335 | 0.687 | 0.247 | 5.18E−119 |
| Lims2 | 5.53E−121 | 0.36535639 | 0.679 | 0.249 | 1.37E−116 |
| Ppara | 1.26E−120 | 0.40400248 | 0.831 | 0.407 | 3.13E−116 |
| Gys2 | 3.37E−120 | 0.43932352 | 0.784 | 0.358 | 8.36E−116 |
| Cyp3a25 | 4.59E−120 | 0.35070342 | 0.995 | 0.919 | 1.14E−115 |
| Nr1i3 | 3.28E−119 | 0.47025715 | 0.814 | 0.377 | 8.14E−115 |
| Decr2 | 1.73E−118 | 0.45805699 | 0.92 | 0.634 | 4.30E−114 |
| Atp5b | 2.24E−118 | 0.34893252 | 0.997 | 0.942 | 5.56E−114 |
| Cox8a | 4.00E−118 | 0.54660995 | 0.89 | 0.621 | 9.92E−114 |
| Gm2a | 4.51E−118 | 0.43890359 | 0.827 | 0.427 | 1.12E−113 |
| Atp5h | 7.92E−118 | 0.53194908 | 0.933 | 0.682 | 1.97E−113 |
| Acad11 | 1.10E−117 | 0.4378614 | 0.896 | 0.564 | 2.74E−113 |
| Ephx2 | 1.34E−117 | 0.3598736 | 0.993 | 0.937 | 3.32E−113 |
| Hspa5 | 3.19E−117 | 0.40850657 | 0.991 | 0.944 | 7.93E−113 |
| Scand1 | 4.17E−117 | 0.25755493 | 0.579 | 0.174 | 1.04E−112 |
| Sdhc | 2.61E−116 | 0.45765895 | 0.91 | 0.63 | 6.47E−112 |
| Slc25a20 | 4.35E−115 | 0.4293254 | 0.877 | 0.498 | 1.08E−110 |
| Uroc1 | 7.32E−115 | 0.50396621 | 0.914 | 0.615 | 1.82E−110 |
| Amy1 | 1.28E−114 | 0.44291653 | 0.943 | 0.676 | 3.19E−110 |
| Mlycd | 3.29E−114 | 0.43192633 | 0.893 | 0.503 | 8.18E−110 |
| Slc27a2 | 4.49E−114 | 0.37825624 | 0.996 | 0.949 | 1.12E−109 |
| Gstt3 | 5.33E−114 | 0.36201954 | 0.737 | 0.286 | 1.32E−109 |
| Gyk | 4.07E−113 | 0.37405715 | 0.778 | 0.35 | 1.01E−108 |
| Cox5a | 6.86E−113 | 0.45488054 | 0.938 | 0.702 | 1.70E−108 |
| Ugt2b5 | 2.21E−111 | 0.33506188 | 0.996 | 0.959 | 5.49E−107 |
| Mgll | 3.59E−111 | 0.40884648 | 0.844 | 0.449 | 8.92E−107 |
| Khk | 5.46E−111 | 0.4591056 | 0.982 | 0.863 | 1.36E−106 |
| Rpl4 | 1.15E−110 | 0.3674266 | 0.987 | 0.915 | 2.85E−106 |
| Fah | 4.69E−109 | 0.39558346 | 0.974 | 0.834 | 1.17E−104 |
| Echs1 | 7.81E−109 | 0.45110855 | 0.971 | 0.817 | 1.94E−104 |
| Brap | 1.53E−108 | 0.38180614 | 0.829 | 0.427 | 3.80E−104 |
| Hdac11 | 4.53E−108 | 0.32069886 | 0.713 | 0.285 | 1.13E−103 |
| Bnip3 | 1.03E−107 | 0.45283659 | 0.876 | 0.55 | 2.56E−103 |
| Slc40a1 | 1.04E−107 | 0.3734929 | 0.744 | 0.328 | 2.58E−103 |
| Ddb1 | 2.29E−107 | 0.39083919 | 0.83 | 0.428 | 5.69E−103 |
| Inhbc | 4.12E−107 | 0.25374724 | 0.595 | 0.201 | 1.02E−102 |
| Armc1 | 2.37E−106 | 0.26734554 | 0.632 | 0.225 | 5.88E−102 |
| Scp2 | 2.42E−106 | −0.4139053 | 0.995 | 0.986 | 6.01E−102 |
| Mup12 | 2.57E−106 | −0.7129524 | 0.754 | 0.86 | 6.39E−102 |
| Camk2n1 | 1.71E−105 | 0.30984563 | 0.691 | 0.278 | 4.25E−101 |
| Lpin2 | 2.11E−105 | 0.46846516 | 0.921 | 0.597 | 5.23E−101 |
| Saa1 | 3.09E−105 | −1.0130216 | 0.393 | 0.662 | 7.67E−101 |
| Acat1 | 3.61E−105 | 0.38666145 | 0.978 | 0.838 | 8.97E−101 |
| Sfxn2 | 4.28E−105 | 0.27640229 | 0.654 | 0.243 | 1.06E−100 |
| Slc25a10 | 1.78E−104 | 0.41888519 | 0.877 | 0.536 | 4.43E−100 |
| Snord13 | 2.03E−104 | 0.25675581 | 0.559 | 0.175 | 5.04E−100 |
| mt-Co3 | 3.45E−104 | −0.8113661 | 0.213 | 0.603 | 8.56E−100 |
| Pigr | 1.34E−103 | 0.47648784 | 0.977 | 0.947 | 3.33E−99 |
| Hspa8 | 2.84E−103 | 0.41559393 | 0.952 | 0.756 | 7.06E−99 |
| Tmbim4 | 4.95E−103 | 0.38382176 | 0.784 | 0.394 | 1.23E−98 |
| Agpat6 | 1.27E−102 | 0.38404305 | 0.884 | 0.517 | 3.14E−98 |
| Ppp2r5e | 3.79E−102 | 0.29726616 | 0.662 | 0.259 | 9.40E−98 |
| Serpina1e | 9.56E−102 | −0.3734273 | 0.998 | 0.999 | 2.37E−97 |
| Gltscr2 | 1.21E−101 | 0.34501105 | 0.83 | 0.424 | 3.00E−97 |
| Atp5k | 6.53E−101 | 0.41069121 | 0.935 | 0.678 | 1.62E−96 |
| Klf9 | 1.72E−100 | 0.38955613 | 0.862 | 0.52 | 4.28E−96 |
| Tcea3 | 1.98E−100 | 0.40437799 | 0.825 | 0.439 | 4.93E−96 |
| Ass1 | 3.83E−100 | −0.4198124 | 0.978 | 0.962 | 9.51E−96 |
| Prdx1 | 4.65E−100 | −0.4756182 | 0.937 | 0.927 | 1.15E−95 |
| Kng1 | 6.40E−100 | 0.25435632 | 0.999 | 0.991 | 1.59E−95 |
| Slc37a4 | 7.88E−100 | 0.41531041 | 0.858 | 0.502 | 1.96E−95 |
| Ctsh | 9.91E−99 | 0.43587811 | 0.945 | 0.727 | 2.46E−94 |
| Serpinf1 | 4.39E−98 | 0.37187623 | 0.976 | 0.864 | 1.09E−93 |
| Yipf3 | 8.87E−98 | 0.29415639 | 0.68 | 0.275 | 2.20E−93 |
| Lipa | 1.27E−97 | 0.4009047 | 0.904 | 0.61 | 3.16E−93 |
| Azgp1 | 1.30E−97 | 0.27232649 | 0.997 | 0.968 | 3.22E−93 |
| Hsd3b5 | 1.31E−97 | −0.8595063 | 0.301 | 0.587 | 3.26E−93 |
| D17Wsu92e | 3.43E−96 | 0.38410204 | 0.909 | 0.588 | 8.53E−92 |
| Abhd14b | 3.92E−96 | 0.39606641 | 0.885 | 0.565 | 9.72E−92 |
| C4b | 4.24E−96 | 0.41301977 | 0.975 | 0.883 | 1.05E−91 |
| Zfp91 | 1.40E−95 | 0.36762155 | 0.855 | 0.492 | 3.48E−91 |
| Gfra1 | 2.52E−95 | 0.37425606 | 0.867 | 0.498 | 6.26E−91 |
| 0610005C13Rik | 2.86E−95 | 0.38034455 | 0.944 | 0.653 | 7.11E−91 |
| Tm4sf4 | 1.44E−94 | 0.2596724 | 0.658 | 0.256 | 3.56E−90 |
| Smoc1 | 1.84E−94 | 0.26326942 | 0.626 | 0.238 | 4.56E−90 |
| Sec14l2 | 1.15E−93 | 0.36294512 | 0.98 | 0.856 | 2.85E−89 |
| F12 | 1.52E−93 | 0.37294854 | 0.966 | 0.814 | 3.77E−89 |
| Pitpnb | 1.52E−93 | 0.34010954 | 0.795 | 0.393 | 3.78E−89 |
| BC025446 | 1.65E−93 | 0.30101942 | 0.714 | 0.306 | 4.10E−89 |
| Acot12 | 2.15E−93 | 0.31850968 | 0.825 | 0.425 | 5.33E−89 |
| Tgoln1 | 3.18E−93 | 0.39559469 | 0.891 | 0.575 | 7.90E−89 |
| Arhgap5 | 1.96E−92 | 0.34695663 | 0.852 | 0.454 | 4.86E−88 |
| Fcgrt | 3.70E−92 | 0.36904045 | 0.91 | 0.599 | 9.20E−88 |
| Fabp1 | 6.83E−92 | −0.3049262 | 0.999 | 1 | 1.70E−87 |
| Eci2 | 2.39E−91 | 0.36362519 | 0.944 | 0.698 | 5.93E−87 |
| Sec61a1 | 2.87E−91 | 0.3812213 | 0.825 | 0.476 | 7.12E−87 |
| Aco2 | 6.42E−91 | 0.3485193 | 0.846 | 0.493 | 1.59E−86 |
| Cdip1 | 3.73E−90 | 0.25310032 | 0.674 | 0.273 | 9.26E−86 |
| Ctsa | 3.79E−90 | 0.30415135 | 0.731 | 0.33 | 9.41E−86 |
| Etnppl | 5.20E−90 | 0.57021741 | 0.866 | 0.606 | 1.29E−85 |
| Lrg1 | 1.16E−89 | 0.39241541 | 0.945 | 0.736 | 2.88E−85 |
| Abca6 | 1.96E−89 | 0.37574506 | 0.849 | 0.507 | 4.87E−85 |
| Aldoa | 3.68E−89 | 0.2857358 | 0.719 | 0.316 | 9.15E−85 |
| Gabarap | 4.31E−89 | 0.37626669 | 0.956 | 0.769 | 1.07E−84 |
| Pbld1 | 1.46E−88 | 0.35826568 | 0.881 | 0.565 | 3.64E−84 |
| Mia3 | 9.36E−88 | 0.37197189 | 0.91 | 0.61 | 2.33E−83 |
| Sfxn1 | 3.26E−87 | 0.41219077 | 0.822 | 0.496 | 8.09E−83 |
| Cyp1a2 | 1.61E−86 | −0.6807537 | 0.567 | 0.794 | 4.00E−82 |
| Ifnar2 | 1.94E−86 | 0.2837916 | 0.731 | 0.328 | 4.81E−82 |
| Acadl | 9.49E−86 | 0.33585482 | 0.916 | 0.612 | 2.36E−81 |
| Serinc3 | 1.50E−85 | 0.34287593 | 0.793 | 0.433 | 3.73E−81 |
| Apoc1 | 1.95E−85 | 0.28437006 | 1 | 0.999 | 4.84E−81 |
| Cyp2c44 | 3.72E−85 | 0.43648833 | 0.924 | 0.698 | 9.23E−81 |
| Actn4 | 9.93E−85 | 0.33312677 | 0.789 | 0.425 | 2.47E−80 |
| Asgr2 | 1.03E−84 | 0.3550777 | 0.879 | 0.555 | 2.56E−80 |
| Abcc3 | 1.65E−84 | 0.39254854 | 0.859 | 0.542 | 4.10E−80 |
| Atf5 | 2.70E−84 | 0.3780825 | 0.909 | 0.641 | 6.70E−80 |
| Tmem219 | 3.14E−84 | 0.26964524 | 0.664 | 0.28 | 7.80E−80 |
| Hpx | 3.75E−84 | 0.3181157 | 0.999 | 0.997 | 9.32E−80 |
| G0s2 | 7.32E−84 | 0.39032412 | 0.692 | 0.33 | 1.82E−79 |
| Shmt1 | 1.69E−83 | 0.35115019 | 0.882 | 0.556 | 4.20E−79 |
| Mrpl43 | 1.78E−83 | 0.26402895 | 0.714 | 0.316 | 4.42E−79 |
| Ggnbp2 | 1.97E−83 | 0.31843548 | 0.818 | 0.43 | 4.89E−79 |
| Cyp2d26 | 3.17E−83 | 0.31789903 | 0.982 | 0.903 | 7.88E−79 |
| Acbd5 | 3.49E−83 | 0.34046005 | 0.943 | 0.708 | 8.65E−79 |
| Atxn7l3b | 3.75E−83 | 0.32661693 | 0.846 | 0.476 | 9.30E−79 |
| Cab39l | 3.82E−83 | 0.26219157 | 0.733 | 0.331 | 9.50E−79 |
| Mtdh | 7.86E−83 | 0.28994736 | 0.819 | 0.428 | 1.95E−78 |
| Creb3l3 | 7.93E−83 | 0.3329936 | 0.971 | 0.831 | 1.97E−78 |
| Slc10a1 | 1.05E−82 | 0.37161916 | 0.984 | 0.843 | 2.61E−78 |
| Ech1 | 2.53E−82 | 0.35576676 | 0.98 | 0.856 | 6.29E−78 |
| Furin | 3.12E−82 | 0.3476705 | 0.781 | 0.417 | 7.75E−78 |
| Cpt2 | 7.06E−82 | 0.3131465 | 0.807 | 0.436 | 1.75E−77 |
| Ctsd | 8.31E−82 | 0.35186323 | 0.916 | 0.656 | 2.06E−77 |
| Asl | 2.52E−81 | 0.45960679 | 0.953 | 0.787 | 6.25E−77 |
| Lamp1 | 6.63E−81 | 0.33067129 | 0.982 | 0.885 | 1.65E−76 |
| Slc2a2 | 6.96E−81 | 0.37119647 | 0.824 | 0.479 | 1.73E−76 |
| Gpld1 | 5.01E−80 | 0.34918584 | 0.877 | 0.577 | 1.24E−75 |
| Bche | 1.32E−79 | 0.29962103 | 0.752 | 0.369 | 3.27E−75 |
| Gcdh | 2.44E−79 | 0.31260712 | 0.977 | 0.85 | 6.06E−75 |
| Dhcr24 | 2.63E−79 | 0.38999534 | 0.953 | 0.783 | 6.54E−75 |
| Maob | 2.50E−78 | 0.36130489 | 0.889 | 0.575 | 6.21E−74 |
| Mttp | 3.08E−78 | 0.34245511 | 0.933 | 0.697 | 7.64E−74 |
| Clu | 4.53E−78 | 0.26617797 | 0.997 | 0.958 | 1.12E−73 |
| Cox6a1 | 8.00E−78 | 0.32671096 | 0.987 | 0.887 | 1.99E−73 |
| Surf4 | 2.41E−77 | 0.32708715 | 0.873 | 0.551 | 5.98E−73 |
| Suds3 | 4.51E−77 | 0.27997975 | 0.74 | 0.364 | 1.12E−72 |
| Igf1 | 1.75E−76 | 0.34278741 | 0.985 | 0.904 | 4.34E−72 |
| Phlda1 | 2.53E−76 | 0.30670303 | 0.72 | 0.356 | 6.29E−72 |
| Akr1d1 | 6.54E−76 | 0.34507376 | 0.852 | 0.515 | 1.62E−71 |
| Snx3 | 8.85E−76 | 0.26415091 | 0.693 | 0.324 | 2.20E−71 |
| Pcbp2 | 9.01E−76 | 0.30951078 | 0.778 | 0.438 | 2.24E−71 |
| Slc4a4 | 2.41E−75 | 0.26414508 | 0.708 | 0.336 | 5.99E−71 |
| Tmed5 | 2.77E−75 | 0.31719235 | 0.86 | 0.492 | 6.87E−71 |
| Hgfac | 2.82E−75 | 0.30206409 | 0.786 | 0.438 | 7.00E−71 |
| 1600002H07Rik | 4.78E−75 | 0.26043978 | 0.736 | 0.367 | 1.19E−70 |
| Atp1a1 | 6.00E−75 | 0.31879405 | 0.8 | 0.457 | 1.49E−70 |
| Aspdh | 9.40E−75 | 0.29976949 | 0.776 | 0.411 | 2.34E−70 |
| Atp2a2 | 9.70E−75 | 0.34965213 | 0.805 | 0.495 | 2.41E−70 |
| Ivns1abp | 5.96E−74 | 0.25484425 | 0.685 | 0.315 | 1.48E−69 |
| Ubxn4 | 2.33E−73 | 0.31590951 | 0.893 | 0.579 | 5.79E−69 |
| Pcx | 3.13E−73 | 0.33904029 | 0.912 | 0.661 | 7.78E−69 |
| Acadvl | 4.86E−73 | 0.30727305 | 0.959 | 0.783 | 1.21E−68 |
| Hnf4a | 6.29E−73 | 0.34260828 | 0.94 | 0.743 | 1.56E−68 |
| Pbld2 | 7.71E−73 | 0.30880678 | 0.891 | 0.586 | 1.91E−68 |
| Slc25a13 | 2.11E−72 | 0.3099101 | 0.924 | 0.655 | 5.25E−68 |
| Ahcy | 2.94E−72 | 0.25978038 | 0.988 | 0.916 | 7.30E−68 |
| Oplah | 3.87E−72 | 0.30158653 | 0.805 | 0.455 | 9.61E−68 |
| Cebpb | 4.19E−72 | 0.29703786 | 0.815 | 0.45 | 1.04E−67 |
| Sord | 8.74E−72 | 0.31435807 | 0.987 | 0.931 | 2.17E−67 |
| Eific | 9.45E−72 | 0.29343555 | 0.868 | 0.541 | 2.35E−67 |
| Pck1 | 1.11E−71 | 0.35910816 | 0.999 | 0.987 | 2.76E−67 |
| Park7 | 5.64E−71 | 0.33535943 | 0.953 | 0.784 | 1.40E−66 |
| Lifr | 9.77E−71 | −0.6366246 | 0.673 | 0.694 | 2.43E−66 |
| Npc2 | 1.08E−70 | 0.25651649 | 0.767 | 0.397 | 2.69E−66 |
| C130074G19Rik | 1.40E−70 | 0.27256617 | 0.803 | 0.439 | 3.48E−66 |
| Sdhd | 1.50E−70 | 0.40776118 | 0.891 | 0.677 | 3.73E−66 |
| Ctage5 | 1.61E−70 | 0.33481901 | 0.941 | 0.683 | 4.00E−66 |
| Atp5g3 | 5.36E−70 | 0.30300905 | 0.985 | 0.911 | 1.33E−65 |
| Man2a1 | 6.00E−70 | 0.33303555 | 0.856 | 0.583 | 1.49E−65 |
| Man2b1 | 6.86E−70 | 0.25308423 | 0.691 | 0.327 | 1.70E−65 |
| Atp5d | 7.13E−70 | 0.3074367 | 0.935 | 0.712 | 1.77E−65 |
| Hsd17b10 | 7.55E−70 | 0.40597546 | 0.927 | 0.7 | 1.87E−65 |
| Sult1d1 | 1.68E−69 | 0.47541266 | 0.936 | 0.684 | 4.17E−65 |
| Cpt1a | 1.92E−69 | 0.3717929 | 0.896 | 0.651 | 4.77E−65 |
| Scrpinf2 | 2.02E−69 | 0.3073114 | 0.966 | 0.843 | 5.01E−65 |
| Map2k2 | 2.13E−69 | 0.28116858 | 0.832 | 0.505 | 5.29E−65 |
| Cyp3a11 | 7.95E−69 | 0.35751032 | 1 | 1 | 1.97E−64 |
| Galk1 | 7.57E−68 | 0.25083271 | 0.718 | 0.353 | 1.88E−63 |
| Os9 | 7.80E−68 | 0.3000957 | 0.911 | 0.663 | 1.94E−63 |
| Stbd1 | 2.40E−67 | 0.25163985 | 0.725 | 0.362 | 5.97E−63 |
| Eef1d | 7.21E−67 | 0.25178984 | 0.795 | 0.431 | 1.79E−62 |
| Creg1 | 7.28E−67 | 0.29261497 | 0.993 | 0.926 | 1.81E−62 |
| Itih1 | 8.18E−67 | 0.31889271 | 0.974 | 0.816 | 2.03E−62 |
| Pnpla8 | 1.55E−66 | 0.28423148 | 0.875 | 0.544 | 3.85E−62 |
| Hadh | 1.60E−66 | 0.31148521 | 0.908 | 0.642 | 3.97E−62 |
| 2810007J24Rik | 1.91E−66 | −0.3323662 | 0.971 | 0.943 | 4.75E−62 |
| Cyp2c70 | 4.18E−66 | −0.5119271 | 0.887 | 0.834 | 1.04E−61 |
| Mup8 | 5.86E−66 | −0.8035587 | 0.099 | 0.373 | 1.45E−61 |
| Cyp7b1 | 8.93E−66 | −0.5490083 | 0.745 | 0.757 | 2.22E−61 |
| C3 | 1.24E−65 | 0.25701603 | 1 | 0.99 | 3.07E−61 |
| Etfb | 4.53E−65 | 0.26087717 | 0.996 | 0.94 | 1.12E−60 |
| Ninj1 | 8.63E−65 | 0.33268814 | 0.874 | 0.6 | 2.14E−60 |
| Eif5 | 2.27E−64 | 0.29265666 | 0.953 | 0.74 | 5.63E−60 |
| Pglyrp2 | 2.64E−64 | 0.28472176 | 0.754 | 0.414 | 6.57E−60 |
| St13 | 2.78E−64 | 0.27490607 | 0.902 | 0.605 | 6.91E−60 |
| BC089597 | 3.62E−64 | 0.32040389 | 0.813 | 0.488 | 9.00E−60 |
| F9 | 7.60E−64 | 0.28721732 | 0.897 | 0.632 | 1.89E−59 |
| Upb1 | 1.23E−63 | 0.30883423 | 0.945 | 0.759 | 3.05E−59 |
| Dnajc3 | 1.92E−63 | 0.27870056 | 0.96 | 0.772 | 4.77E−59 |
| Tomm70a | 2.60E−63 | 0.26465337 | 0.84 | 0.494 | 6.45E−59 |
| Hadha | 7.87E−63 | 0.30184069 | 0.943 | 0.744 | 1.95E−58 |
| Cfi | 8.49E−63 | 0.27302279 | 0.984 | 0.905 | 2.11E−58 |
| Gstp1 | 1.03E−62 | −0.7039865 | 0.816 | 0.822 | 2.56E−58 |
| Nfe211 | 1.25E−62 | 0.25237576 | 0.648 | 0.303 | 3.11E−58 |
| Pccb | 1.80E−62 | 0.28471305 | 0.901 | 0.644 | 4.48E−58 |
| Cd1d1 | 2.74E−62 | 0.31029654 | 0.887 | 0.612 | 6.80E−58 |
| Sec62 | 5.58E−62 | 0.25355237 | 0.891 | 0.582 | 1.38E−57 |
| Ndufb10 | 1.58E−61 | 0.3124426 | 0.907 | 0.681 | 3.92E−57 |
| Fam195a | 3.67E−61 | 0.25895675 | 0.726 | 0.373 | 9.11E−57 |
| Agpat3 | 4.91E−61 | 0.28331345 | 0.874 | 0.606 | 1.22E−56 |
| Usmg5 | 5.77E−61 | 0.34205598 | 0.755 | 0.43 | 1.43E−56 |
| Pla2g12b | 1.63E−60 | 0.26759045 | 0.798 | 0.463 | 4.05E−56 |
| Pim3 | 1.69E−60 | 0.28844058 | 0.845 | 0.537 | 4.19E−56 |
| Rdx | 2.64E−60 | 0.27239982 | 0.918 | 0.63 | 6.57E−56 |
| Itih4 | 4.78E−60 | 0.25619887 | 0.989 | 0.948 | 1.19E−55 |
| Aplp2 | 5.59E−60 | 0.29614531 | 0.89 | 0.625 | 1.39E−55 |
| Tmem176b | 6.55E−60 | 0.2869571 | 0.89 | 0.632 | 1.63E−55 |
| Mst1 | 2.99E−59 | 0.26762035 | 0.81 | 0.491 | 7.42E−55 |
| Pipox | 3.78E−59 | 0.27664192 | 0.849 | 0.549 | 9.39E−55 |
| Hal | 8.00E−59 | 0.41089477 | 0.908 | 0.749 | 1.99E−54 |
| 1-Mar | 9.16E−59 | 0.26809923 | 0.984 | 0.885 | 2.28E−54 |
| C9 | 3.01E−58 | −0.3557254 | 0.958 | 0.938 | 7.47E−54 |
| Apoc4 | 3.29E−58 | 0.31784645 | 0.998 | 0.98 | 8.16E−54 |
| Lman1 | 2.98E−57 | 0.29104793 | 0.908 | 0.655 | 7.40E−53 |
| Slc7a2 | 7.19E−57 | 0.38667853 | 0.921 | 0.735 | 1.78E−52 |
| Gsto1 | 2.33E−56 | 0.29242538 | 0.818 | 0.52 | 5.79E−52 |
| Por | 4.50E−56 | 0.33065864 | 0.921 | 0.675 | 1.12E−51 |
| Fmo5 | 4.62E−56 | 0.25685221 | 0.979 | 0.837 | 1.15E−51 |
| Slc25a39 | 1.37E−55 | 0.28667795 | 0.938 | 0.775 | 3.41E−51 |
| Eef2 | 3.32E−55 | 0.2761614 | 0.976 | 0.873 | 8.24E−51 |
| Ces2e | 3.94E−55 | 0.26453598 | 0.852 | 0.535 | 9.79E−51 |
| Mup14 | 5.64E−55 | −0.7319112 | 0.458 | 0.616 | 1.40E−50 |
| Tmem56 | 6.78E−55 | 0.27132827 | 0.878 | 0.612 | 1.68E−50 |
| Atp5j | 2.12E−54 | 0.31656359 | 0.916 | 0.757 | 5.27E−50 |
| Glrx5 | 2.30E−54 | 0.26880807 | 0.886 | 0.588 | 5.72E−50 |
| Dmgdh | 4.17E−54 | 0.26793926 | 0.952 | 0.764 | 1.04E−49 |
| Hsbp1 | 6.41E−54 | 0.25010647 | 0.792 | 0.498 | 1.59E−49 |
| Hsd17b4 | 1.05E−53 | 0.26350655 | 0.971 | 0.818 | 2.60E−49 |
| Sardh | 2.77E−53 | 0.29292716 | 0.959 | 0.838 | 6.87E−49 |
| Rgn | 3.35E−53 | −0.4023915 | 0.745 | 0.874 | 8.33E−49 |
| Rxra | 3.40E−53 | 0.26022884 | 0.849 | 0.574 | 8.43E−49 |
| Ugp2 | 5.36E−53 | 0.30081989 | 0.945 | 0.749 | 1.33E−48 |
| Fn1 | 4.88E−52 | 0.31062863 | 0.945 | 0.821 | 1.21E−47 |
| Hsd3b7 | 7.74E−52 | −0.4335194 | 0.82 | 0.798 | 1.92E−47 |
| Comt | 9.63E−52 | −0.3017516 | 0.958 | 0.931 | 2.39E−47 |
| Chpt1 | 1.19E−51 | 0.2736267 | 0.912 | 0.664 | 2.96E−47 |
| Itih3 | 1.29E−51 | 0.2835814 | 0.978 | 0.89 | 3.20E−47 |
| Cyp2d22 | 1.40E−51 | 0.25806171 | 0.835 | 0.546 | 3.48E−47 |
| Gpt | 3.62E−51 | 0.25721554 | 0.94 | 0.753 | 8.98E−47 |
| Sds | 5.00E−51 | 0.52218417 | 0.799 | 0.67 | 1.24E−46 |
| Mcfd2 | 7.32E−51 | 0.26358926 | 0.899 | 0.643 | 1.82E−46 |
| 1100001G20Rik | 1.27E−49 | 0.29940469 | 0.993 | 0.982 | 3.17E−45 |
| Ndufs8 | 3.03E−49 | 0.27492092 | 0.854 | 0.607 | 7.54E−45 |
| Dak | 2.23E−48 | 0.31749445 | 0.852 | 0.563 | 5.54E−44 |
| Ndufb2 | 2.40E−48 | 0.29336153 | 0.888 | 0.629 | 5.96E−44 |
| Elovl2 | 9.90E−48 | 0.26623714 | 0.938 | 0.699 | 2.46E−43 |
| Orm1 | 1.29E−47 | −0.3040626 | 0.97 | 0.952 | 3.19E−43 |
| Rplp0 | 5.33E−47 | −0.3888413 | 0.788 | 0.791 | 1.32E−42 |
| Pemt | 1.55E−46 | 0.28354116 | 0.948 | 0.78 | 3.86E−42 |
| C8a | 4.65E−45 | −0.3574056 | 0.897 | 0.887 | 1.15E−40 |
| Akr1c19 | 9.54E−45 | 0.26176816 | 0.821 | 0.527 | 2.37E−40 |
| Oaz1 | 1.95E−44 | −0.4482997 | 0.779 | 0.75 | 4.84E−40 |
| Cyp4a12a | 6.66E−44 | −0.4778521 | 0.463 | 0.597 | 1.65E−39 |
| Serpina3c | 1.23E−43 | −0.5143596 | 0.195 | 0.385 | 3.05E−39 |
| C8b | 1.58E−43 | −0.335278 | 0.953 | 0.881 | 3.93E−39 |
| Cideb | 1.82E−43 | 0.26185632 | 0.892 | 0.676 | 4.52E−39 |
| Col18a1 | 4.74E−41 | 0.25271374 | 0.941 | 0.79 | 1.18E−36 |
| Gsta3 | 2.08E−40 | −0.2675808 | 0.993 | 0.956 | 5.17E−36 |
| Ugt2b1 | 2.65E−40 | −0.3075642 | 0.938 | 0.871 | 6.59E−36 |
| Rsp14 | 1.55E−39 | −0.3000601 | 0.943 | 0.917 | 3.85E−35 |
| Gchfr | 9.84E−39 | 0.25204319 | 0.953 | 0.768 | 2.44E−34 |
| mt-Atp6 | 4.42E−37 | −0.2634338 | 0.234 | 0.452 | 1.10E−32 |
| Mup13 | 2.44E−36 | −0.6330576 | 0.468 | 0.546 | 6.05E−32 |
| Saa2 | 1.64E−34 | −0.3719511 | 0.049 | 0.222 | 4.07E−30 |
| Eef1b2 | 4.92E−33 | −0.3627624 | 0.851 | 0.78 | 1.22E−28 |
| Actb | 7.58E−33 | −0.278801 | 0.938 | 0.886 | 1.88E−28 |
| Cfh | 3.95E−32 | −0.2876493 | 0.943 | 0.897 | 9.80E−28 |
| Sparcl1 | 7.94E−32 | −0.253617 | 0 | 0.136 | 1.97E−27 |
| Slco1a1 | 1.65E−31 | −0.3676845 | 0.865 | 0.777 | 4.10E−27 |
| Uqcrq | 1.55E−30 | −0.2551066 | 0.98 | 0.951 | 3.85E−26 |
| Cyb5b | 2.54E−30 | −0.3686974 | 0.787 | 0.724 | 6.31E−26 |
| Keg1 | 4.30E−30 | −0.5313206 | 0.527 | 0.545 | 1.07E−25 |
| Mup2 | 4.37E−30 | −0.6018783 | 0.254 | 0.392 | 1.09E−25 |
| Gstm3 | 4.79E−29 | −0.6276519 | 0.279 | 0.414 | 1.19E−24 |
| Inmt | 5.27E−29 | −0.3314912 | 0.936 | 0.884 | 1.31E−24 |
| Cml2 | 6.13E−29 | −0.2801974 | 0.905 | 0.857 | 1.52E−24 |
| Ttc39c | 1.40E−28 | −0.3963737 | 0.696 | 0.642 | 3.49E−24 |
| Scd1 | 6.31E−28 | 0.29915098 | 0.982 | 0.901 | 1.57E−23 |
| Mup20 | 1.62E−27 | −0.2683861 | 0.999 | 1 | 4.03E−23 |
| Rps2 | 1.94E−26 | −0.3547845 | 0.775 | 0.757 | 4.81E−22 |
| Egfr | 2.57E−25 | −0.3563097 | 0.908 | 0.809 | 6.38E−21 |
| Cfhr2 | 5.00E−25 | −0.3089882 | 0.946 | 0.868 | 1.24E−20 |
| Thrsp | 6.63E−24 | −0.5876996 | 0.697 | 0.627 | 1.65E−19 |
| Hamp | 1.08E−23 | −0.2994038 | 0.864 | 0.884 | 2.67E−19 |
| Leap2 | 5.80E−23 | 0.25433574 | 0.833 | 0.632 | 1.44E−18 |
| Gm4076 | 8.06E−23 | −0.3148234 | 0.057 | 0.184 | 2.00E−18 |
| Cyp2f2 | 1.22E−22 | 0.25135427 | 0.912 | 0.863 | 3.03E−18 |
| Ankrd55 | 8.13E−22 | −0.7865348 | 0.833 | 0.696 | 2.02E−17 |
| Hsd17b2 | 1.06E−21 | −0.3251185 | 0.767 | 0.711 | 2.63E−17 |
| Igfbp1 | 1.21E−17 | −0.2514632 | 0.744 | 0.717 | 3.01E−13 |
| Slc22a30 | 1.29E−16 | −0.3628576 | 0.497 | 0.493 | 3.21E−12 |
| Cyp7a1 | 2.60E−16 | −0.2737711 | 0.143 | 0.259 | 6.44E−12 |
| Gm20594 | 3.73E−16 | −0.4169188 | 0.186 | 0.294 | 9.27E−12 |
| Rpl32 | 4.05E−16 | −0.3042904 | 0.835 | 0.757 | 1.01E−11 |
| Ndufb11 | 5.15E−16 | −0.2689829 | 0.802 | 0.735 | 1.28E−11 |
| Hspe1 | 3.93E−14 | −0.323232 | 0.582 | 0.557 | 9.76E−10 |
| Rarres1 | 8.16E−13 | −0.2957998 | 0.374 | 0.42 | 2.03E−08 |
| mmu-mir-6236 | 1.23E−12 | −0.2819002 | 0.911 | 0.837 | 3.06E−08 |
| Slco1b2 | 1.73E−12 | 0.27152878 | 0.996 | 0.951 | 4.30E−08 |
| Lect2 | 1.80E−12 | −0.2645229 | 0.318 | 0.414 | 4.48E−08 |
| Tlcd2 | 3.11E−12 | −0.3052345 | 0.573 | 0.536 | 7.73E−08 |
| Bst2 | 1.23E−11 | −0.3075801 | 0.591 | 0.54 | 3.06E−07 |
| Glul | 1.75E−11 | 0.54377904 | 0.308 | 0.18 | 4.36E−07 |
| Cyp2c67 | 2.22E−11 | −0.2853612 | 0.36 | 0.418 | 5.52E−07 |
| Hsd3b3 | 2.39E−11 | −0.2879231 | 0.664 | 0.605 | 5.94E−07 |
| Acss2 | 6.15E−11 | −0.3108047 | 0.685 | 0.574 | 1.53E−06 |
| Mup1 | 1.46E−10 | −0.5088135 | 0.619 | 0.583 | 3.61E−06 |
| Nme1 | 1.61E−10 | −0.2976676 | 0.742 | 0.622 | 4.00E−06 |
| Selk | 1.42E−09 | −0.3116864 | 0.532 | 0.486 | 3.52E−05 |
| C6 | 1.16E−08 | −0.2909762 | 0.725 | 0.642 | 0.00028737 |
| Rps8 | 2.26E−08 | −0.2891652 | 0.502 | 0.471 | 0.00056072 |
| Cxcl1 | 8.10E−08 | −0.2564732 | 0.264 | 0.314 | 0.00201077 |
| Adh6-ps1 | 2.80E−07 | −0.2741169 | 0.284 | 0.313 | 0.00695696 |
| Pnpla7 | 6.51E−07 | −0.2689142 | 0.677 | 0.572 | 0.01616347 |
| 1500017E21Rik | 6.87E−07 | −0.2767539 | 0.511 | 0.476 | 0.01704927 |
| Rpl36a1 | 5.46E−06 | −0.2835914 | 0.643 | 0.551 | 0.13560354 |
| Ndufb4 | 1.01E−05 | −0.262186 | 0.554 | 0.49 | 0.24969401 |
| Rpl34 | 0.00330931 | −0.2548221 | 0.445 | 0.398 | 1 |
| Acly | 0.00429145 | −0.2626893 | 0.537 | 0.445 | 1 |
| Ubb | 0.0845165 | −0.2608023 | 0.542 | 0.45 | 1 |
| Slc1a2 | 0.09580614 | 0.26120345 | 0.055 | 0.042 | 1 |
| Ftl1 | 0.23123934 | −0.3160431 | 0.504 | 0.409 | 1 |
| Rpl41 | 0.66932474 | −0.3294678 | 0.331 | 0.278 | 1 |
| Cyp2b10 | 0.90869564 | −0.2984609 | 0.723 | 0.542 | 1 |

TABLE 4b
Differentially expressed genes between time point A48A96 and untreated (UT).

| ID | p_val | avg_logFC | pct.1 | pct.2 | p_val_adj |
|---|---|---|---|---|---|
| Cyp4a14 | 0 | 1.704093543 | 0.984 | 0.431 | 0 |
| Cyp4a10 | 0 | 1.135370857 | 0.983 | 0.657 | 0 |
| Mat1a | 0 | 1.000433202 | 1 | 0.966 | 0 |
| Apoc2 | 0 | 0.782126085 | 0.943 | 0.802 | 0 |
| Chchd10 | 0 | 0.720691838 | 0.994 | 0.944 | 0 |
| Angptl3 | 0 | 0.7089371 | 0.996 | 0.944 | 0 |
| Hmgcs2 | 0 | 0.629289122 | 0.998 | 0.986 | 0 |
| Apoe | 0 | 0.407256897 | 1 | 1 | 0 |
| Serpina1e | 0 | −0.662045945 | 0.988 | 0.999 | 0 |
| mt-Nd5 | 0 | −0.799147191 | 0.977 | 0.995 | 0 |
| mt-Cytb | 0 | −0.8545555 | 0.993 | 0.999 | 0 |
| mt-Nd2 | 0 | −0.898054789 | 0.994 | 0.999 | 0 |
| mt-Nd4 | 0 | −0.936405909 | 0.992 | 0.998 | 0 |
| Mup21 | 0 | −1.008171912 | 0.729 | 0.929 | 0 |
| mt-Co1 | 0 | −1.043307563 | 0.989 | 0.999 | 0 |
| Gm13775 | 0 | −1.07808822 | 0.311 | 0.761 | 0 |
| Mup16 | 0 | −1.100681479 | 0.646 | 0.934 | 0 |
| Mup7 | 0 | −1.115587848 | 0.934 | 0.998 | 0 |
| Nudt7 | 0 | −1.14157535 | 0.674 | 0.83 | 0 |
| Selenbp2 | 0 | −1.190221952 | 0.715 | 0.937 | 0 |
| Mup18 | 0 | −1.590870011 | 0.906 | 0.997 | 0 |
| Mup11 | 0 | −1.621134615 | 0.9 | 1 | 0 |
| Mup19 | 0 | −1.730868177 | 0.955 | 1 | 0 |
| Mup9 | 0 | −1.93588422 | 0.603 | 0.976 | 0 |
| Mup6 | 0 | −2.032084932 | 0.834 | 0.992 | 0 |
| Mup17 | 0 | −2.09635042 | 0.97 | 1 | 0 |
| Ttpa | 1.7348507452971e−317 | 0.601240096 | 0.986 | 0.869 | 4.30798137072176e−313 |
| Ces1c | 9.12743755309997e−311 | 0.466837177 | 0.999 | 0.985 | 2.27E−306 |
| mt-Nd1 | 2.58E−308 | −0.591646758 | 0.998 | 1 | 6.39E−304 |
| Retsat | 3.87E−303 | 0.681307283 | 0.973 | 0.83 | 9.61E−299 |
| Cdo1 | 1.81E−299 | 0.517045053 | 0.997 | 0.96 | 4.49E−295 |
| mt-Rnr2 | 3.93E−280 | −0.559446336 | 1 | 1 | 9.76E−276 |
| Mup15 | 2.41E−278 | −1.26121256 | 0.184 | 0.617 | 5.98E−274 |
| Apoh | 1.27E−276 | 0.411790107 | 0.998 | 0.986 | 3.16E−272 |
| Saa1 | 2.92E−263 | −1.078220383 | 0.318 | 0.662 | 7.26E−259 |
| Hsd3b5 | 7.75E−256 | −0.830145941 | 0.277 | 0.587 | 1.92E−251 |
| Mup5 | 1.09E−253 | −1.351810718 | 0.6 | 0.839 | 2.70E−249 |
| Mup3 | 3.11E−251 | −0.379596014 | 1 | 1 | 7.72E−247 |
| Apoc1 | 1.38E−241 | 0.392095407 | 1 | 0.999 | 3.42E−237 |
| Sepp1 | 9.85E−239 | 0.33970689 | 1 | 1 | 2.45E−234 |
| Sult1a1 | 3.66E−236 | 0.560453494 | 0.924 | 0.707 | 9.10E−232 |
| Hrg | 8.23E−233 | 0.518895355 | 0.957 | 0.831 | 2.04E−228 |
| Mfsd2a | 2.44E−230 | 0.612232714 | 0.58 | 0.148 | 6.07E−226 |
| Agt | 9.72E−229 | 0.520454244 | 0.949 | 0.817 | 2.41E−224 |
| mt-Rnr1 | 5.60E−214 | −0.485399855 | 1 | 1 | 1.39E−209 |
| Ankrd55 | 2.17E−211 | −0.974280427 | 0.678 | 0.696 | 5.38E−207 |
| Ndrg2 | 2.43E−211 | 0.41219937 | 0.993 | 0.939 | 6.03E−207 |
| Mup12 | 6.39E−210 | −0.767698995 | 0.668 | 0.86 | 1.59E−205 |
| Lifr | 1.86E−209 | −0.563681141 | 0.618 | 0.694 | 4.62E−205 |
| Thrsp | 4.77E−209 | −0.689057582 | 0.576 | 0.627 | 1.18E−204 |
| Gstp1 | 6.36E−208 | −0.855831436 | 0.681 | 0.822 | 1.58E−203 |
| Snord118 | 6.27E−207 | 0.617425052 | 0.671 | 0.248 | 1.56E−202 |
| Ahsg | 7.66E−203 | 0.292489576 | 1 | 1 | 1.90E−198 |
| mt-Co3 | 7.66E−203 | −0.915108473 | 0.213 | 0.603 | 1.90E−198 |
| Ttr | 1.30E−198 | 0.388907438 | 1 | 1 | 3.23E−194 |
| Slco1a4 | 5.66E−197 | 0.552641329 | 0.646 | 0.238 | 1.41E−192 |
| Herpud1 | 2.45E−196 | 0.513571228 | 0.945 | 0.803 | 6.08E−192 |
| mt-Co2 | 1.16E−193 | −0.918784455 | 0.221 | 0.629 | 2.88E−189 |
| Keg1 | 4.42E−188 | −0.521611435 | 0.457 | 0.545 | 1.10E−183 |
| Igfbp4 | 1.13E−187 | 0.43528879 | 0.978 | 0.866 | 2.80E−183 |
| Akr1c6 | 5.38E−186 | −0.468988487 | 0.96 | 0.971 | 1.34E−181 |
| Reep6 | 2.43E−184 | 0.514272451 | 0.931 | 0.794 | 6.05E−180 |
| Cp | 4.92E−184 | 0.436243259 | 0.994 | 0.943 | 1.22E−179 |
| Aldob | 1.31E−183 | 0.365460648 | 0.999 | 0.996 | 3.24E−179 |
| Abcd2 | 1.68E−177 | 0.285090597 | 0.366 | 0.052 | 4.17E−173 |
| Abcb4 | 6.95E−177 | 0.494736917 | 0.919 | 0.675 | 1.73E−172 |
| Gm23935 | 7.08E−176 | 0.50194422 | 0.994 | 0.925 | 1.76E−171 |
| Cyp7b1 | 1.38E−173 | −0.542403458 | 0.67 | 0.757 | 3.43E−169 |
| St3gal5 | 4.05E−172 | 0.542378437 | 0.818 | 0.49 | 1.01E−167 |
| Slc25a47 | 5.63E−172 | 0.502661475 | 0.977 | 0.927 | 1.40E−167 |
| Decr1 | 2.17E−169 | 0.466364339 | 0.889 | 0.694 | 5.38E−165 |
| Fth1 | 3.96E−168 | 0.378367166 | 0.994 | 0.978 | 9.82E−164 |
| Slc27a2 | 3.59E−167 | 0.385256962 | 0.992 | 0.949 | 8.93E−163 |
| Pabpc1 | 2.52E−164 | 0.494914201 | 0.844 | 0.592 | 6.27E−160 |
| Ttc36 | 5.61E−163 | 0.38128834 | 0.988 | 0.95 | 1.39E−158 |
| Rdh7 | 4.71E−160 | 0.364161562 | 0.995 | 0.969 | 1.17E−155 |
| Mup8 | 1.32E−159 | −0.79575704 | 0.084 | 0.373 | 3.27E−155 |
| Lcat | 5.85E−158 | 0.436082968 | 0.932 | 0.781 | 1.45E−153 |
| Gc | 7.12E−157 | 0.259816642 | 1 | 0.999 | 1.77E−152 |
| Ube2r2 | 3.35E−153 | 0.46659877 | 0.819 | 0.525 | 8.31E−149 |
| Hmgcl | 4.83E−152 | 0.473794522 | 0.916 | 0.729 | 1.20E−147 |
| Grn | 5.47E−152 | 0.473252349 | 0.743 | 0.396 | 1.36E−147 |
| Mup20 | 7.34E−152 | −0.476207303 | 0.998 | 1 | 1.82E−147 |
| Mup4 | 2.86E−151 | −0.953101828 | 0.801 | 0.898 | 7.09E−147 |
| Apoc3 | 2.12E−150 | 0.327008256 | 1 | 0.993 | 5.26E−146 |
| Aldh9a1 | 5.77E−148 | 0.415934269 | 0.926 | 0.763 | 1.43E−143 |
| Hspa8 | 8.18E−148 | 0.420379148 | 0.93 | 0.756 | 2.03E−143 |
| Egfr | 9.84E−147 | −0.473388687 | 0.806 | 0.809 | 2.44E−142 |
| F2 | 2.22E−145 | 0.32133198 | 0.998 | 0.969 | 5.51E−141 |
| Ttc39c | 1.58E−142 | −0.374504475 | 0.625 | 0.642 | 3.91E−138 |
| Rbp4 | 1.96E−142 | 0.301440617 | 1 | 0.999 | 4.88E−138 |
| Serpina3c | 3.94E−141 | −0.472511045 | 0.201 | 0.385 | 9.79E−137 |
| Amy1 | 4.69E−141 | 0.432738347 | 0.89 | 0.676 | 1.16E−136 |
| Serpina3m | 6.01E−140 | 0.432583404 | 0.885 | 0.722 | 1.49E−135 |
| Elovl3 | 2.08E−139 | −0.442652848 | 0.736 | 0.827 | 5.17E−135 |
| Apoa5 | 1.53E−138 | 0.323188383 | 0.993 | 0.974 | 3.80E−134 |
| Serping1 | 6.04E−137 | 0.41280952 | 0.96 | 0.87 | 1.50E−132 |
| Rpl38 | 7.83E−137 | 0.254329206 | 0.482 | 0.16 | 1.95E−132 |
| Cyp2c70 | 9.28E−137 | −0.395158527 | 0.859 | 0.834 | 2.30E−132 |
| Gyk | 2.15E−136 | 0.402444184 | 0.702 | 0.35 | 5.33E−132 |
| Crat | 2.27E−136 | 0.255626614 | 0.474 | 0.15 | 5.64E−132 |
| C9 | 6.07E−134 | −0.427055258 | 0.926 | 0.938 | 1.51E−129 |
| Wbp1l | 2.25E−133 | 0.440462928 | 0.853 | 0.606 | 5.58E−129 |
| Gstt3 | 2.25E−133 | 0.350739865 | 0.645 | 0.286 | 5.59E−129 |
| Decr2 | 5.16E−133 | 0.418851877 | 0.87 | 0.634 | 1.28E−128 |
| Tmbim6 | 9.26E−133 | 0.338652766 | 0.992 | 0.942 | 2.30E−128 |
| Mup14 | 2.38E−132 | −0.760766854 | 0.4 | 0.616 | 5.90E−128 |
| Ildr2 | 7.46E−131 | 0.267607713 | 0.479 | 0.159 | 1.85E−126 |
| Atp5h | 1.29E−130 | 0.434163543 | 0.893 | 0.682 | 3.20E−126 |
| Nr1i3 | 1.67E−129 | 0.425883248 | 0.725 | 0.377 | 4.15E−125 |
| Cyp3a25 | 2.41E−129 | 0.318573266 | 0.988 | 0.919 | 5.98E−125 |
| Lipa | 2.95E−129 | 0.41833763 | 0.851 | 0.61 | 7.32E−125 |
| Oaz1 | 3.92E−129 | −0.418528822 | 0.715 | 0.75 | 9.74E−125 |
| Oaf | 1.31E−128 | 0.403567608 | 0.928 | 0.794 | 3.26E−124 |
| Scd1 | 1.93E−128 | 0.519218079 | 0.985 | 0.901 | 4.78E−124 |
| Atp5k | 2.65E−128 | 0.408878378 | 0.889 | 0.678 | 6.59E−124 |
| Plin5 | 2.96E−128 | 0.352087102 | 0.667 | 0.315 | 7.34E−124 |
| Rgs16 | 3.78E−128 | 0.322559157 | 0.415 | 0.113 | 9.40E−124 |
| Prdx1 | 1.61E−127 | −0.361890861 | 0.934 | 0.927 | 4.01E−123 |
| Cox5a | 3.24E−127 | 0.40236048 | 0.88 | 0.702 | 8.04E−123 |
| Car3 | 2.20E−125 | −0.405308591 | 0.97 | 0.974 | 5.47E−121 |
| Asl | 4.60E−125 | 0.438458339 | 0.926 | 0.787 | 1.14E−120 |
| Hsd3b7 | 1.98E−124 | −0.382738321 | 0.777 | 0.798 | 4.92E−120 |
| Acat1 | 3.21E−124 | 0.368871999 | 0.949 | 0.838 | 7.96E−120 |
| Mup13 | 5.23E−124 | −0.55255936 | 0.444 | 0.546 | 1.30E−119 |
| Rplp0 | 6.61E−124 | −0.430901519 | 0.707 | 0.791 | 1.64E−119 |
| Acbd5 | 2.77E−123 | 0.385608793 | 0.909 | 0.708 | 6.87E−119 |
| Agpat6 | 2.85E−123 | 0.396260224 | 0.817 | 0.517 | 7.07E−119 |
| Cox8a | 9.05E−122 | 0.44171223 | 0.818 | 0.621 | 2.25E−117 |
| Bfar | 3.09E−121 | 0.290332897 | 0.61 | 0.27 | 7.66E−117 |
| Vtn | 6.34E−121 | 0.333811366 | 0.998 | 0.983 | 1.57E−116 |
| C8b | 3.83E−117 | −0.360171247 | 0.894 | 0.881 | 9.52E−113 |
| Sdhc | 2.51E−116 | 0.394392518 | 0.839 | 0.63 | 6.24E−112 |
| Slc27a5 | 3.14E−116 | 0.336994904 | 0.973 | 0.893 | 7.79E−112 |
| Dhcr24 | 8.17E−116 | 0.387274317 | 0.944 | 0.783 | 2.03E−111 |
| Snord13 | 2.01E−113 | 0.261078586 | 0.478 | 0.175 | 4.98E−109 |
| Ppara | 6.59E−113 | 0.35885744 | 0.729 | 0.407 | 1.64E−108 |
| Slc25a20 | 8.33E−113 | 0.380017899 | 0.78 | 0.498 | 2.07E−108 |
| Slc40a1 | 3.89E−111 | 0.356026755 | 0.653 | 0.328 | 9.66E−107 |
| Acss2 | 2.82E−110 | −0.258362678 | 0.611 | 0.574 | 7.00E−106 |
| Brap | 4.77E−110 | 0.349229859 | 0.741 | 0.427 | 1.19E−105 |
| Sult1d1 | 1.78E−109 | 0.45987026 | 0.898 | 0.684 | 4.42E−105 |
| Tgoln1 | 6.82E−109 | 0.38833408 | 0.828 | 0.575 | 1.69E−104 |
| Slc4a4 | 9.50E−109 | 0.331670131 | 0.661 | 0.336 | 2.36E−104 |
| D17Wsu92e | 2.20E−108 | 0.371951808 | 0.844 | 0.588 | 5.47E−104 |
| Gm6484 | 4.91E−108 | 0.304268201 | 0.472 | 0.174 | 1.22E−103 |
| Acot12 | 5.33E−107 | 0.307840041 | 0.743 | 0.425 | 1.32E−102 |
| Vnn3 | 8.28E−107 | 0.276458974 | 0.555 | 0.241 | 2.06E−102 |
| Bnip3 | 2.17E−106 | 0.403206563 | 0.8 | 0.55 | 5.38E−102 |
| Sh3glb1 | 7.00E−106 | 0.326006747 | 0.571 | 0.258 | 1.74E−101 |
| Tmed5 | 1.13E−105 | 0.345007803 | 0.792 | 0.492 | 2.80E−101 |
| Mia3 | 2.04E−105 | 0.364623797 | 0.863 | 0.61 | 5.08E−101 |
| Gm2a | 2.30E−105 | 0.383109253 | 0.714 | 0.427 | 5.71E−101 |
| Lpin2 | 3.75E−105 | 0.385908326 | 0.855 | 0.597 | 9.32E−101 |
| Slco1a1 | 2.50E−104 | −0.295640242 | 0.823 | 0.777 | 6.20E−100 |
| Sec14l2 | 2.77E−104 | 0.321333578 | 0.961 | 0.856 | 6.87E−100 |
| Atp5b | 6.90E−104 | 0.291727476 | 0.99 | 0.942 | 1.71E−99 |
| Zfp91 | 1.18E−103 | 0.370686172 | 0.779 | 0.492 | 2.92E−99 |
| Cyp2a12 | 4.26E−103 | 0.374332361 | 0.952 | 0.884 | 1.06E−98 |
| 0610005C13Rik | 5.95E−103 | 0.34237016 | 0.884 | 0.653 | 1.48E−98 |
| Uroc1 | 1.08E−102 | 0.416226092 | 0.825 | 0.615 | 2.68E−98 |
| Nme1 | 5.87E−102 | −0.285705103 | 0.657 | 0.622 | 1.46E−97 |
| Mtdh | 1.64E−101 | 0.30466184 | 0.739 | 0.428 | 4.08E−97 |
| Eef1b2 | 4.83E−101 | −0.317323205 | 0.8 | 0.78 | 1.20E−96 |
| Acad11 | 1.54E−100 | 0.357135026 | 0.809 | 0.564 | 3.83E−96 |
| Ass1 | 2.76E−100 | −0.314400604 | 0.971 | 0.962 | 6.84E−96 |
| Camk2n1 | 4.05E−100 | 0.250109512 | 0.586 | 0.278 | 1.01E−95 |
| Akr1d1 | 5.03E−100 | 0.361037667 | 0.79 | 0.515 | 1.25E−95 |
| Dennd5b | 8.13E−99 | 0.266493539 | 0.643 | 0.334 | 2.02E−94 |
| Ephx2 | 1.62E−98 | 0.281258562 | 0.981 | 0.937 | 4.03E−94 |
| Scp2 | 1.84E−98 | −0.325161752 | 0.988 | 0.986 | 4.57E−94 |
| Serpinf1 | 3.42E−98 | 0.319550979 | 0.956 | 0.864 | 8.50E−94 |
| Acadl | 6.56E−98 | 0.316216015 | 0.862 | 0.612 | 1.63E−93 |
| Slc22a30 | 7.73E−98 | −0.295400819 | 0.461 | 0.493 | 1.92E−93 |
| Cpt1a | 3.10E−97 | 0.372935212 | 0.868 | 0.651 | 7.70E−93 |
| Cyp2c44 | 7.78E−97 | 0.385935584 | 0.879 | 0.698 | 1.93E−92 |
| Mgll | 9.00E−97 | 0.333899751 | 0.743 | 0.449 | 2.24E−92 |
| Ces1b | 9.68E−97 | 0.372958858 | 0.717 | 0.437 | 2.40E−92 |
| Chpt1 | 1.79E−96 | 0.35662056 | 0.882 | 0.664 | 4.45E−92 |
| Bche | 2.34E−96 | 0.313419985 | 0.675 | 0.369 | 5.82E−92 |
| Hes6 | 7.85E−96 | −0.262263942 | 0.415 | 0.42 | 1.95E−91 |
| BC025446 | 8.95E−96 | 0.272610935 | 0.611 | 0.306 | 2.22E−91 |
| Gstm3 | 1.32E−95 | −0.598573335 | 0.256 | 0.414 | 3.28E−91 |
| Slc25a10 | 2.78E−95 | 0.343272504 | 0.794 | 0.536 | 6.91E−91 |
| Abcc3 | 4.53E−95 | 0.363799323 | 0.8 | 0.542 | 1.13E−90 |
| Rsp14 | 7.49E−95 | −0.348405505 | 0.908 | 0.917 | 1.86E−90 |
| Gfra1 | 1.15E−94 | 0.320843264 | 0.782 | 0.498 | 2.86E−90 |
| Rdx | 1.46E−94 | 0.318521875 | 0.874 | 0.63 | 3.62E−90 |
| Pex11a | 1.66E−94 | 0.350827626 | 0.738 | 0.441 | 4.11E−90 |
| Tmbim4 | 2.87E−94 | 0.32269634 | 0.689 | 0.394 | 7.12E−90 |
| Selk | 1.39E−93 | −0.289300641 | 0.467 | 0.486 | 3.45E−89 |
| Rpl4 | 3.80E−93 | 0.283935593 | 0.977 | 0.915 | 9.43E−89 |
| Aplp2 | 4.37E−93 | 0.33938781 | 0.86 | 0.625 | 1.08E−88 |
| Mup2 | 1.14E−92 | −0.55305847 | 0.255 | 0.392 | 2.83E−88 |
| Apoa4 | 8.99E−92 | 0.495579268 | 0.62 | 0.339 | 2.23E−87 |
| Pnpla7 | 9.47E−92 | −0.301626901 | 0.572 | 0.572 | 2.35E−87 |
| Kif1b | 2.14E−91 | 0.299610278 | 0.732 | 0.435 | 5.33E−87 |
| Serp1 | 2.20E−91 | −0.267566027 | 0.76 | 0.721 | 5.46E−87 |
| CtagcS | 1.97E−90 | 0.328678207 | 0.892 | 0.683 | 4.89E−86 |
| Fah | 6.12E−90 | 0.302494707 | 0.939 | 0.834 | 1.52E−85 |
| Hsd17b13 | 1.23E−89 | 0.550399594 | 0.85 | 0.74 | 3.06E−85 |
| Tmem56 | 4.57E−89 | 0.335238196 | 0.847 | 0.612 | 1.14E−84 |
| Comt | 6.00E−89 | −0.286248465 | 0.932 | 0.931 | 1.49E−84 |
| Slc10a1 | 7.28E−89 | 0.314325805 | 0.961 | 0.843 | 1.81E−84 |
| Sparcl1 | 9.28E−89 | −0.253616986 | 0 | 0.136 | 2.30E−84 |
| Pbld1 | 3.56E−88 | 0.322688203 | 0.807 | 0.565 | 8.83E−84 |
| Hsd17b2 | 5.55E−88 | −0.313480586 | 0.695 | 0.711 | 1.38E−83 |
| Abca6 | 6.11E−88 | 0.33284944 | 0.773 | 0.507 | 1.52E−83 |
| Saa2 | 4.41E−87 | −0.390082382 | 0.036 | 0.222 | 1.10E−82 |
| Mettl7b | 2.12E−86 | 0.283916114 | 0.963 | 0.869 | 5.26E−82 |
| Atxn7l3b | 2.41E−86 | 0.306239775 | 0.756 | 0.476 | 5.98E−82 |
| Gpld1 | 3.69E−86 | 0.328106789 | 0.816 | 0.577 | 9.16E−82 |
| Cfhr2 | 4.83E−86 | −0.306325678 | 0.917 | 0.868 | 1.20E−81 |
| Hsd3b3 | 1.64E−85 | −0.299662144 | 0.586 | 0.605 | 4.08E−81 |
| G0s2 | 2.06E−85 | 0.346452213 | 0.614 | 0.33 | 5.12E−81 |
| Prodh2 | 3.05E−85 | 0.345294809 | 0.812 | 0.611 | 7.57E−81 |
| Por | 6.21E−85 | 0.33194923 | 0.891 | 0.675 | 1.54E−80 |
| Prdx4 | 1.37E−84 | −0.251975081 | 0.642 | 0.607 | 3.41E−80 |
| Hsd17b10 | 2.19E−84 | 0.361579378 | 0.887 | 0.7 | 5.43E−80 |
| Mttp | 2.30E−84 | 0.311504107 | 0.878 | 0.697 | 5.72E−80 |
| Serpina3n | 2.31E−84 | 0.346482791 | 0.788 | 0.574 | 5.73E−80 |
| Hspa5 | 2.40E−84 | 0.291184144 | 0.984 | 0.944 | 5.96E−80 |
| Creg1 | 5.44E−84 | 0.276773054 | 0.99 | 0.926 | 1.35E−79 |
| Cyp3a11 | 1.17E−83 | 0.313927334 | 0.999 | 1 | 2.91E−79 |
| Pnpla8 | 2.68E−83 | 0.295631611 | 0.803 | 0.544 | 6.66E−79 |
| Ctsa | 2.92E−83 | 0.252009224 | 0.615 | 0.33 | 7.25E−79 |
| Insig2 | 5.58E−83 | 0.300825179 | 0.95 | 0.81 | 1.39E−78 |
| C8a | 6.02E−83 | −0.327131388 | 0.882 | 0.887 | 1.49E−78 |
| Echs1 | 7.04E−83 | 0.318692311 | 0.935 | 0.817 | 1.75E−78 |
| Pigr | 1.00E−82 | 0.386842112 | 0.967 | 0.947 | 2.48E−78 |
| Eif5 | 2.11E−82 | 0.284340466 | 0.918 | 0.74 | 5.24E−78 |
| Hnf4a | 3.28E−82 | 0.314710574 | 0.901 | 0.743 | 8.15E−78 |
| Etnppl | 3.81E−82 | 0.496712405 | 0.777 | 0.606 | 9.45E−78 |
| Arhgap5 | 4.87E−82 | 0.288514066 | 0.731 | 0.454 | 1.21E−77 |
| Eci2 | 5.30E−82 | 0.311004843 | 0.877 | 0.698 | 1.31E−77 |
| Nfe2l1 | 1.57E−81 | 0.27034473 | 0.582 | 0.303 | 3.89E−77 |
| Pcbp2 | 2.09E−81 | 0.310347013 | 0.704 | 0.438 | 5.18E−77 |
| Actb | 2.95E−81 | −0.294140078 | 0.892 | 0.886 | 7.34E−77 |
| Cyp1a2 | 4.20E−81 | −0.445766232 | 0.638 | 0.794 | 1.04E−76 |
| Actn4 | 5.06E−81 | 0.299765947 | 0.7 | 0.425 | 1.26E−76 |
| Igf1 | 9.43E−81 | 0.295791825 | 0.973 | 0.904 | 2.34E−76 |
| Cox6a1 | 2.01E−80 | 0.292128773 | 0.966 | 0.887 | 5.00E−76 |
| Tcea3 | 2.70E−80 | 0.3163181 | 0.709 | 0.439 | 6.70E−76 |
| Gltscr2 | 4.30E−80 | 0.255310647 | 0.702 | 0.424 | 1.07E−75 |
| Gcdh | 5.44E−80 | 0.273385895 | 0.95 | 0.85 | 1.35E−75 |
| Orm1 | 1.32E−79 | −0.31003929 | 0.947 | 0.952 | 3.28E−75 |
| Atf5 | 1.56E−79 | 0.329004772 | 0.832 | 0.641 | 3.87E−75 |
| Creb3l3 | 2.61E−79 | 0.275700825 | 0.939 | 0.831 | 6.48E−75 |
| Ctsh | 5.03E−79 | 0.32445036 | 0.883 | 0.727 | 1.25E−74 |
| Cxcl12 | 1.28E−78 | 0.257250112 | 0.696 | 0.419 | 3.17E−74 |
| Cbr1 | 2.47E−78 | 0.260731659 | 0.672 | 0.395 | 6.15E−74 |
| Mlycd | 2.57E−78 | 0.313046697 | 0.754 | 0.503 | 6.38E−74 |
| Aco2 | 2.89E−78 | 0.285855791 | 0.752 | 0.493 | 7.18E−74 |
| Maob | 6.31E−78 | 0.32037326 | 0.804 | 0.575 | 1.57E−73 |
| Slc25a13 | 1.28E−77 | 0.285473174 | 0.859 | 0.655 | 3.18E−73 |
| Man2a1 | 1.37E−77 | 0.332755834 | 0.8 | 0.583 | 3.41E−73 |
| Slco1b2 | 1.49E−77 | 0.386023002 | 0.997 | 0.951 | 3.69E−73 |
| Rps2 | 1.93E−77 | −0.374246561 | 0.697 | 0.757 | 4.80E−73 |
| Tomm70a | 3.24E−77 | 0.265762726 | 0.76 | 0.494 | 8.04E−73 |
| Ggnbp2 | 7.84E−77 | 0.272604502 | 0.703 | 0.43 | 1.95E−72 |
| Asgr2 | 1.23E−76 | 0.306445529 | 0.785 | 0.555 | 3.06E−72 |
| Gabarap | 1.23E−76 | 0.289400057 | 0.909 | 0.769 | 3.06E−72 |
| F10 | 1.47E−76 | 0.279095541 | 0.96 | 0.895 | 3.65E−72 |
| Cyp2d26 | 1.49E−76 | 0.259136874 | 0.969 | 0.903 | 3.71E−72 |
| Mt1 | 3.77E−76 | 0.356326784 | 0.81 | 0.556 | 9.36E−72 |
| Serinc3 | 4.38E−76 | 0.292320724 | 0.697 | 0.433 | 1.09E−71 |
| C130074G19Rik | 4.54E−76 | 0.250322698 | 0.71 | 0.439 | 1.13E−71 |
| Suds3 | 8.94E−75 | 0.25388963 | 0.635 | 0.364 | 2.22E−70 |
| Ddb1 | 1.79E−74 | 0.277809186 | 0.694 | 0.428 | 4.44E−70 |
| Ces1d | 3.46E−74 | 0.285254637 | 0.98 | 0.923 | 8.60E−70 |
| Rps8 | 1.82E−73 | −0.274601282 | 0.444 | 0.471 | 4.51E−69 |
| P4hb | 3.56E−73 | 0.250660872 | 0.984 | 0.941 | 8.83E−69 |
| Tob1 | 7.22E−73 | 0.284985634 | 0.693 | 0.43 | 1.79E−68 |
| Hadh | 7.72E−73 | 0.288772268 | 0.845 | 0.642 | 1.92E−68 |
| C3 | 8.36E−73 | 0.259166718 | 0.999 | 0.99 | 2.08E−68 |
| Slc7a2 | 1.29E−72 | 0.34497807 | 0.884 | 0.735 | 3.21E−68 |
| Gys2 | 2.56E−72 | 0.274401611 | 0.623 | 0.358 | 6.36E−68 |
| Klf9 | 2.74E−72 | 0.293415875 | 0.756 | 0.52 | 6.80E−68 |
| Ei24 | 2.82E−72 | 0.257327011 | 0.738 | 0.48 | 7.01E−68 |
| Sec62 | 4.20E−72 | 0.251575572 | 0.82 | 0.582 | 1.04E−67 |
| Dmgdh | 5.63E−72 | 0.265846058 | 0.923 | 0.764 | 1.40E−67 |
| Ppib | 7.64E−72 | −0.260118322 | 0.793 | 0.767 | 1.90E−67 |
| Abhd14b | 2.59E−71 | 0.299363281 | 0.78 | 0.565 | 6.42E−67 |
| Sord | 7.97E−71 | 0.26850985 | 0.978 | 0.931 | 1.98E−66 |
| Furin | 1.72E−70 | 0.278163716 | 0.674 | 0.417 | 4.27E−66 |
| Shmt1 | 6.32E−70 | 0.278820727 | 0.788 | 0.556 | 1.57E−65 |
| Ugp2 | 8.02E−70 | 0.297335445 | 0.904 | 0.749 | 1.99E−65 |
| Lamp1 | 9.89E−70 | 0.268283663 | 0.959 | 0.885 | 2.46E−65 |
| Khk | 1.54E−69 | 0.310103866 | 0.963 | 0.863 | 3.82E−65 |
| Cd1d1 | 2.00E−69 | 0.291156805 | 0.821 | 0.612 | 4.96E−65 |
| Fmo5 | 3.51E−69 | 0.252070931 | 0.959 | 0.837 | 8.71E−65 |
| C4b | 5.80E−69 | 0.283273876 | 0.953 | 0.883 | 1.44E−64 |
| Mup1 | 1.20E−68 | −0.556836871 | 0.525 | 0.583 | 2.97E−64 |
| Hspe1 | 1.32E−68 | −0.281318916 | 0.533 | 0.557 | 3.28E−64 |
| Rdh16 | 1.89E−68 | 0.269182372 | 0.525 | 0.272 | 4.69E−64 |
| Ndufb11 | 9.28E−68 | −0.275645983 | 0.739 | 0.735 | 2.30E−63 |
| Atp2a2 | 1.64E−67 | 0.305636215 | 0.728 | 0.495 | 4.07E−63 |
| Ces3b | 1.96E−67 | −0.263292817 | 0.792 | 0.787 | 4.87E−63 |
| Acsl1 | 2.51E−67 | 0.256335416 | 0.977 | 0.924 | 6.24E−63 |
| Ftl1 | 3.38E−67 | −0.29880847 | 0.438 | 0.409 | 8.39E−63 |
| Lpgat1 | 1.67E−66 | 0.257600415 | 0.747 | 0.502 | 4.15E−62 |
| Hsd17b4 | 4.01E−66 | 0.253232967 | 0.943 | 0.818 | 9.96E−62 |
| Ifitm3 | 8.45E−66 | −0.254348675 | 0.823 | 0.812 | 2.10E−61 |
| Tmem106b | 1.27E−65 | 0.270357032 | 0.787 | 0.557 | 3.16E−61 |
| Gclm | 1.64E−65 | 0.258765469 | 0.893 | 0.712 | 4.08E−61 |
| F9 | 4.58E−65 | 0.256805166 | 0.836 | 0.632 | 1.14E−60 |
| Pipox | 5.65E−65 | 0.272543042 | 0.774 | 0.549 | 1.40E−60 |
| Atp1a1 | 6.53E−65 | 0.262770413 | 0.701 | 0.457 | 1.62E−60 |
| Sardh | 9.29E−65 | 0.269183147 | 0.939 | 0.838 | 2.31E−60 |
| Gsta2 | 1.03E−64 | 0.252161786 | 0.583 | 0.333 | 2.57E−60 |
| Sdhd | 2.03E−64 | 0.320841923 | 0.837 | 0.677 | 5.04E−60 |
| Gabarapl1 | 4.17E−64 | 0.260716513 | 0.917 | 0.779 | 1.04E−59 |
| Inmt | 5.29E−64 | −0.25322177 | 0.935 | 0.884 | 1.31E−59 |
| Usmg5 | 2.56E−63 | 0.278730923 | 0.677 | 0.43 | 6.37E−59 |
| Hgfac | 3.33E−63 | 0.252189991 | 0.682 | 0.438 | 8.26E−59 |
| F12 | 5.05E−63 | 0.256737037 | 0.922 | 0.814 | 1.25E−58 |
| Ubxn4 | 6.14E−63 | 0.250313647 | 0.802 | 0.579 | 1.52E−58 |
| Rpl41 | 8.17E−63 | −0.294952328 | 0.304 | 0.278 | 2.03E−58 |
| Slc2a2 | 2.10E−62 | 0.272297971 | 0.716 | 0.479 | 5.21E−58 |
| Fcgrt | 2.17E−62 | 0.258478842 | 0.806 | 0.599 | 5.39E−58 |
| Rpl32 | 2.50E−62 | −0.284689184 | 0.766 | 0.757 | 6.20E−58 |
| Ubb | 3.95E−62 | −0.264432797 | 0.465 | 0.45 | 9.81E−58 |
| Fn1 | 4.84E−62 | 0.292522175 | 0.932 | 0.821 | 1.20E−57 |
| F5 | 5.57E−61 | 0.300566587 | 0.881 | 0.728 | 1.38E−56 |
| Ptms | 1.52E−60 | 0.267838974 | 0.857 | 0.689 | 3.78E−56 |
| Pck1 | 1.63E−60 | 0.271859881 | 0.998 | 0.987 | 4.04E−56 |
| Rarres1 | 1.78E−60 | −0.291851226 | 0.327 | 0.42 | 4.42E−56 |
| Hadha | 1.24E−59 | 0.250937029 | 0.895 | 0.744 | 3.08E−55 |
| Atp5d | 2.30E−59 | 0.250062798 | 0.866 | 0.712 | 5.70E−55 |
| Gm4076 | 9.59E−59 | −0.308321155 | 0.047 | 0.184 | 2.38E−54 |
| Ctsd | 1.22E−58 | 0.257862212 | 0.831 | 0.656 | 3.04E−54 |
| Sfxn1 | 3.83E−58 | 0.304885886 | 0.702 | 0.496 | 9.52E−54 |
| Ces1e | 3.91E−58 | 0.265618893 | 0.791 | 0.576 | 9.71E−54 |
| C6 | 1.21E−57 | −0.26698766 | 0.653 | 0.642 | 3.00E−53 |
| Upb1 | 1.58E−57 | 0.25038758 | 0.895 | 0.759 | 3.92E−53 |
| Ppp2r5a | 1.67E−57 | 0.250897706 | 0.803 | 0.594 | 4.14E−53 |
| Ech1 | 2.49E−57 | 0.250481301 | 0.951 | 0.856 | 6.18E−53 |
| Sec61a1 | 4.01E−57 | 0.263729835 | 0.7 | 0.476 | 9.95E−53 |
| Slc37a4 | 2.56E−56 | 0.268318739 | 0.717 | 0.502 | 6.35E−52 |
| Cyp8b1 | 2.75E−56 | 0.282315072 | 0.905 | 0.745 | 6.82E−52 |
| Park7 | 6.56E−54 | 0.251517689 | 0.902 | 0.784 | 1.63E−49 |
| Ndufb2 | 2.41E−50 | 0.251595204 | 0.814 | 0.629 | 5.98E−46 |
| Serpina12 | 2.13E−49 | −0.324744036 | 0.38 | 0.492 | 5.28E−45 |
| Cyp4a12a | 3.63E−48 | −0.277051162 | 0.509 | 0.597 | 9.00E−44 |
| Lpin1 | 2.48E−45 | 0.277494868 | 0.515 | 0.31 | 6.16E−41 |
| Gm20594 | 3.69E−41 | −0.356558629 | 0.222 | 0.294 | 9.16E−37 |
| Cyp2c54 | 3.26E−38 | 0.250387024 | 0.879 | 0.759 | 8.10E−34 |
| mmu-mir-6236 | 2.88E−35 | −0.429737333 | 0.859 | 0.837 | 7.15E−31 |
| Sds | 1.58E−34 | 0.297273523 | 0.671 | 0.67 | 3.93E−30 |
| mt-Atp6 | 1.02E−32 | −0.323426357 | 0.326 | 0.452 | 2.52E−28 |
| Hal | 1.03E−25 | 0.280113531 | 0.824 | 0.749 | 2.55E−21 |
| Aldh3a2 | 7.57E−18 | 0.307877353 | 0.592 | 0.529 | 1.88E−13 |
| Glul | 6.83E−11 | 0.379494728 | 0.257 | 0.18 | 1.70E−06 |

TABLE 4c
Differentially expressed genes between time point A96 and untreated (UT)

| I.D. | p_val | avg_logFC | pct.1 | pct.2 | p_val_adj |
|---|---|---|---|---|---|
| Cyp4a14 | 0 | 1.600758457 | 0.975 | 0.431 | 0 |
| Mat1a | 0 | 0.932795094 | 1 | 0.966 | 0 |
| Serpina1e | 0 | −1.134217923 | 0.977 | 0.999 | 0 |
| Mup11 | 0 | −1.558844676 | 0.905 | 1 | 0 |
| Mup9 | 0 | −1.81783443 | 0.619 | 0.976 | 0 |
| Mup17 | 0 | −1.889311872 | 0.98 | 1 | 0 |
| Mup6 | 0 | −2.021370562 | 0.778 | 0.992 | 0 |
| Cyp4a10 | 2.14E−272 | 1.120330137 | 0.974 | 0.657 | 5.30E−268 |
| Angptl3 | 1.84E−268 | 0.693633109 | 0.998 | 0.944 | 4.57E−264 |
| Selenbp2 | 3.56E−267 | −1.257537093 | 0.612 | 0.937 | 8.83E−263 |
| Apoc1 | 1.21E−236 | 0.500838397 | 1 | 0.999 | 3.00E−232 |
| Mup7 | 6.32E−222 | −1.145234189 | 0.938 | 0.998 | 1.57E−217 |
| Mup18 | 6.63E−216 | −1.29677101 | 0.928 | 0.997 | 1.65E−211 |
| Mup16 | 1.30E−213 | −1.118868702 | 0.567 | 0.934 | 3.24E−209 |
| Cdo1 | 2.68E−208 | 0.567928382 | 0.998 | 0.96 | 6.65E−204 |
| Chchd10 | 2.87E−191 | 0.607996934 | 0.994 | 0.944 | 7.12E−187 |
| Serpina3k | 3.14E−181 | −0.303639552 | 1 | 1 | 7.78E−177 |
| Apoe | 3.87E−176 | 0.327046521 | 1 | 1 | 9.61E−172 |
| mt-Nd4 | 4.55E−175 | −0.848768513 | 0.986 | 0.998 | 1.13E−170 |
| Ttpa | 2.91E−171 | 0.626576167 | 0.98 | 0.869 | 7.23E−167 |
| Mup20 | 4.60E−169 | −0.777084754 | 0.996 | 1 | 1.14E−164 |
| Mup21 | 9.94E−166 | −0.940012218 | 0.676 | 0.929 | 2.47E−161 |
| Mup19 | 8.02E−165 | −1.257832573 | 0.973 | 1 | 1.99E−160 |
| Scd1 | 3.56E−164 | 0.719867652 | 0.989 | 0.901 | 8.84E−160 |
| Mup3 | 4.46E−164 | −0.409813908 | 1 | 1 | 1.11E−159 |
| Ttr | 2.21E−163 | 0.345887189 | 1 | 1 | 5.48E−159 |
| mt-Co1 | 9.06E−161 | −0.845943199 | 0.983 | 0.999 | 2.25E−156 |
| mt-Nd2 | 7.76E−159 | −0.71965104 | 0.994 | 0.999 | 1.93E−154 |
| Gm13775 | 2.84E−157 | −1.050178147 | 0.3 | 0.761 | 7.06E−153 |
| Hmgcs2 | 9.48E−157 | 0.423416551 | 0.996 | 0.986 | 2.35E−152 |
| Nudt7 | 3.13E−150 | −1.139673094 | 0.602 | 0.83 | 7.78E−146 |
| mt-Cytb | 2.05E−145 | −0.710470319 | 0.99 | 0.999 | 5.10E−141 |
| Apoh | 3.17E−136 | 0.346721981 | 0.998 | 0.986 | 7.86E−132 |
| Gstp1 | 4.53E−134 | −1.05972633 | 0.529 | 0.822 | 1.12E−129 |
| mt-Nd1 | 9.87E−133 | −0.584321112 | 0.996 | 1 | 2.45E−128 |
| Mup15 | 1.46E−130 | −1.238690862 | 0.156 | 0.617 | 3.63E−126 |
| Saa1 | 2.60E−130 | −1.157027866 | 0.232 | 0.662 | 6.45E−126 |
| Serpina1b | 2.05E−129 | −0.384477147 | 0.996 | 0.996 | 5.10E−125 |
| Ces1c | 2.77E−127 | 0.358303072 | 1 | 0.985 | 6.87E−123 |
| Mup12 | 3.42E−124 | −0.833089417 | 0.571 | 0.86 | 8.50E−120 |
| Mup5 | 2.13E−120 | −1.26562348 | 0.538 | 0.839 | 5.30E−116 |
| Sepp1 | 3.10E−118 | 0.28385063 | 1 | 1 | 7.69E−114 |
| mt-Co3 | 6.00E−105 | −1.046313505 | 0.213 | 0.603 | 1.49E−100 |
| Zfp683 | 2.36E−104 | 0.302737948 | 0.476 | 0.116 | 5.87E−100 |
| Slc27a2 | 2.28E−102 | 0.393073566 | 0.988 | 0.949 | 5.65E−98 |
| Ndrg2 | 1.79E−100 | 0.380210534 | 0.985 | 0.939 | 4.45E−96 |
| Retsat | 4.63E−99 | 0.533337889 | 0.957 | 0.83 | 1.15E−94 |
| Sult1a1 | 2.51E−94 | 0.521965916 | 0.881 | 0.707 | 6.23E−90 |
| Mt1 | 5.36E−94 | 0.637988242 | 0.839 | 0.556 | 1.33E−89 |
| Igfbp4 | 8.30E−94 | 0.418865013 | 0.964 | 0.866 | 2.06E−89 |
| mt-Nd5 | 2.10E−93 | −0.573016379 | 0.964 | 0.995 | 5.21E−89 |
| Fth1 | 7.38E−93 | 0.322617334 | 0.993 | 0.978 | 1.83E−88 |
| Apoc3 | 7.54E−93 | 0.300246662 | 1 | 0.993 | 1.87E−88 |
| C9 | 1.49E−92 | −0.513884034 | 0.89 | 0.938 | 3.69E−88 |
| Serpina1c | 3.40E−91 | −0.317587699 | 0.998 | 0.998 | 8.44E−87 |
| Ces1d | 4.28E−91 | 0.427718067 | 0.977 | 0.923 | 1.06E−86 |
| Hsd3b5 | 2.20E−88 | −0.798118541 | 0.25 | 0.587 | 5.45E−84 |
| Snord118 | 1.26E−87 | 0.578283559 | 0.564 | 0.248 | 3.12E−83 |
| Abcb4 | 3.27E−82 | 0.47584587 | 0.878 | 0.675 | 8.12E−78 |
| mt-Co2 | 1.30E−79 | −1.033164507 | 0.313 | 0.629 | 3.24E−75 |
| Ankrd55 | 9.32E−79 | −1.238650283 | 0.503 | 0.696 | 2.31E−74 |
| mt-Tp | 2.27E−78 | 0.308472055 | 0.329 | 0.083 | 5.63E−74 |
| Hrg | 2.56E−78 | 0.40951941 | 0.923 | 0.831 | 6.37E−74 |
| Slco1b2 | 7.71E−78 | 0.50085864 | 0.998 | 0.951 | 1.91E−73 |
| Hspa8 | 1.28E−76 | 0.425734559 | 0.905 | 0.756 | 3.19E−72 |
| Egfr | 1.83E−75 | −0.623880361 | 0.691 | 0.809 | 4.55E−71 |
| Apoc2 | 6.30E−75 | 0.518676481 | 0.901 | 0.802 | 1.57E−70 |
| Mettl7b | 2.67E−74 | 0.378385546 | 0.949 | 0.869 | 6.64E−70 |
| Pltp | 4.26E−74 | 0.291854911 | 0.237 | 0.045 | 1.06E−69 |
| Crot | 1.15E−73 | 0.332587455 | 0.989 | 0.942 | 2.86E−69 |
| Acbd5 | 2.82E−73 | 0.434071255 | 0.87 | 0.708 | 6.99E−69 |
| Chpt1 | 1.82E−68 | 0.442418636 | 0.848 | 0.664 | 4.53E−64 |
| Mfsd2a | 2.72E−68 | 0.380198802 | 0.41 | 0.148 | 6.75E−64 |
| Gyk | 4.10E−68 | 0.43344086 | 0.616 | 0.35 | 1.02E−63 |
| Atp5k | 5.72E−68 | 0.40683529 | 0.837 | 0.678 | 1.42E−63 |
| Slco1a4 | 2.83E−67 | 0.465182557 | 0.507 | 0.238 | 7.02E−63 |
| Cyp3a11 | 4.60E−67 | 0.262520214 | 0.999 | 1 | 1.14E−62 |
| Agt | 9.05E−67 | 0.376513419 | 0.912 | 0.817 | 2.25E−62 |
| Mup8 | 1.05E−66 | −0.78705351 | 0.067 | 0.373 | 2.61E−62 |
| Lipa | 1.52E−66 | 0.437590517 | 0.791 | 0.61 | 3.78E−62 |
| Asl | 1.71E−66 | 0.414122541 | 0.896 | 0.787 | 4.26E−62 |
| Aldh2 | 1.95E−66 | 0.304046608 | 0.995 | 0.973 | 4.84E−62 |
| Mup14 | 4.19E−66 | −0.794252292 | 0.335 | 0.616 | 1.04E−61 |
| Amy1 | 3.46E−64 | 0.421164278 | 0.831 | 0.676 | 8.59E−60 |
| Rdh7 | 4.17E−64 | 0.267142418 | 0.995 | 0.969 | 1.04E−59 |
| Ube2r2 | 5.94E−64 | 0.424389458 | 0.747 | 0.525 | 1.48E−59 |
| mt-Rnr1 | 2.84E−63 | −0.339795422 | 1 | 1 | 7.04E−59 |
| Ubc | 3.45E−63 | 0.357786823 | 0.968 | 0.872 | 8.58E−59 |
| Thrsp | 5.64E−62 | −0.816880004 | 0.44 | 0.627 | 1.40E−57 |
| C3 | 5.68E−62 | 0.261580397 | 0.998 | 0.99 | 1.41E−57 |
| mt-Rnr2 | 1.26E−61 | −0.333163796 | 1 | 1 | 3.12E−57 |
| Slc4a4 | 3.45E−61 | 0.402545626 | 0.609 | 0.336 | 8.57E−57 |
| Pex11a | 7.21E−61 | 0.462020512 | 0.7 | 0.441 | 1.79E−56 |
| Phyh | 9.71E−61 | 0.254119045 | 0.998 | 0.986 | 2.41E−56 |
| Tmem56 | 3.46E−60 | 0.402560106 | 0.812 | 0.612 | 8.58E−56 |
| Rplp0 | 1.41E−59 | −0.480428304 | 0.617 | 0.791 | 3.50E−55 |
| Agpat6 | 1.75E−59 | 0.409826612 | 0.743 | 0.517 | 4.34E−55 |
| Cyp7b1 | 1.13E−58 | −0.535025845 | 0.585 | 0.757 | 2.81E−54 |
| Acot4 | 2.69E−58 | 0.268017172 | 0.328 | 0.105 | 6.69E−54 |
| Aplp2 | 6.80E−58 | 0.385893889 | 0.826 | 0.625 | 1.69E−53 |
| Insig2 | 9.53E−58 | 0.35940331 | 0.928 | 0.81 | 2.37E−53 |
| Aldh9a1 | 1.53E−57 | 0.363277407 | 0.884 | 0.763 | 3.81E−53 |
| Rsp14 | 2.79E−57 | −0.405734932 | 0.869 | 0.917 | 6.92E−53 |
| Tmed5 | 3.82E−57 | 0.375398251 | 0.714 | 0.492 | 9.48E−53 |
| Dhcr24 | 7.47E−57 | 0.384204715 | 0.933 | 0.783 | 1.85E−52 |
| Akr1c6 | 9.53E−57 | −0.359039536 | 0.967 | 0.971 | 2.37E−52 |
| Cpt1a | 1.90E−56 | 0.374218574 | 0.837 | 0.651 | 4.73E−52 |
| Cp | 2.07E−56 | 0.282591145 | 0.993 | 0.943 | 5.14E−52 |
| Cyp8b1 | 8.62E−56 | 0.416953547 | 0.897 | 0.745 | 2.14E−51 |
| Decr2 | 1.16E−55 | 0.372833523 | 0.813 | 0.634 | 2.89E−51 |
| Sult1d1 | 2.45E−55 | 0.442093823 | 0.855 | 0.684 | 6.09E−51 |
| Tmbim6 | 2.64E−55 | 0.262388341 | 0.988 | 0.942 | 6.56E−51 |
| Acat1 | 7.06E−55 | 0.348475979 | 0.917 | 0.838 | 1.75E−50 |
| Rdx | 5.04E−54 | 0.367980343 | 0.824 | 0.63 | 1.25E−49 |
| Crat | 5.43E−54 | 0.262436474 | 0.389 | 0.15 | 1.35E−49 |
| Cyp3a25 | 3.67E−53 | 0.281153449 | 0.981 | 0.919 | 9.11E−49 |
| Prlr | 6.12E−53 | 0.361079861 | 0.574 | 0.316 | 1.52E−48 |
| Kif1b | 6.31E−53 | 0.390898595 | 0.686 | 0.435 | 1.57E−48 |
| Tgoln1 | 2.31E−52 | 0.380103515 | 0.756 | 0.575 | 5.74E−48 |
| Akr1d1 | 1.08E−51 | 0.378695209 | 0.721 | 0.515 | 2.68E−47 |
| Grn | 1.32E−51 | 0.373090696 | 0.629 | 0.396 | 3.27E−47 |
| Acsl1 | 3.10E−51 | 0.291538271 | 0.977 | 0.924 | 7.69E−47 |
| Lcat | 4.49E−51 | 0.329386736 | 0.886 | 0.781 | 1.12E−46 |
| C8b | 5.02E−51 | −0.388933382 | 0.827 | 0.881 | 1.25E−46 |
| Pabpc1 | 1.07E−50 | 0.369312616 | 0.766 | 0.592 | 2.66E−46 |
| Hmgcl | 1.10E−50 | 0.352973298 | 0.871 | 0.729 | 2.72E−46 |
| Serpina12 | 9.77E−50 | −0.532616287 | 0.226 | 0.492 | 2.43E−45 |
| Mia3 | 1.63E−49 | 0.35629328 | 0.81 | 0.61 | 4.06E−45 |
| Ces3a | 2.69E−49 | −0.284381139 | 0.97 | 0.967 | 6.69E−45 |
| Tob1 | 3.05E−49 | 0.373100971 | 0.649 | 0.43 | 7.58E−45 |
| Gstt3 | 3.52E−49 | 0.337898035 | 0.541 | 0.286 | 8.74E−45 |
| Trp53inp1 | 4.76E−49 | 0.364652102 | 0.551 | 0.308 | 1.18E−44 |
| St3gal5 | 7.54E−48 | 0.38618033 | 0.71 | 0.49 | 1.87E−43 |
| Nr1i3 | 8.17E−48 | 0.373496949 | 0.625 | 0.377 | 2.03E−43 |
| Serpina1a | 3.10E−47 | −0.270847162 | 0.991 | 0.991 | 7.70E−43 |
| Hpx | 3.37E−47 | −0.264031877 | 0.998 | 0.997 | 8.37E−43 |
| D17Wsu92e | 4.32E−47 | 0.358105893 | 0.771 | 0.588 | 1.07E−42 |
| Wbp1l | 1.07E−46 | 0.345645814 | 0.789 | 0.606 | 2.66E−42 |
| Gclm | 2.21E−46 | 0.328178558 | 0.865 | 0.712 | 5.50E−42 |
| Nfe2l2 | 5.21E−46 | 0.304823522 | 0.465 | 0.229 | 1.29E−41 |
| Lifr | 6.77E−46 | −0.487526847 | 0.556 | 0.694 | 1.68E−41 |
| Atp5h | 1.28E−45 | 0.311372918 | 0.848 | 0.682 | 3.18E−41 |
| Cox5a | 2.96E−45 | 0.339787139 | 0.813 | 0.702 | 7.36E−41 |
| Cxcl12 | 7.95E−45 | 0.337162418 | 0.635 | 0.419 | 1.97E−40 |
| F5 | 1.42E−44 | 0.355721719 | 0.858 | 0.728 | 3.52E−40 |
| Zfp91 | 1.78E−44 | 0.374122215 | 0.693 | 0.492 | 4.42E−40 |
| Mtdh | 5.57E−44 | 0.32095866 | 0.649 | 0.428 | 1.38E−39 |
| Hhex | 8.35E−44 | 0.320918894 | 0.543 | 0.307 | 2.07E−39 |
| Acadl | 1.56E−43 | 0.293654435 | 0.8 | 0.612 | 3.88E−39 |
| Ppp2r5a | 2.98E−43 | 0.34926081 | 0.764 | 0.594 | 7.41E−39 |
| Creg1 | 5.93E−43 | 0.258648182 | 0.985 | 0.926 | 1.47E−38 |
| Slc40a1 | 6.14E−43 | 0.336008423 | 0.551 | 0.328 | 1.52E−38 |
| Cpeb4 | 6.82E−43 | 0.341793976 | 0.527 | 0.299 | 1.69E−38 |
| Sec14l2 | 8.99E−43 | 0.272359641 | 0.939 | 0.856 | 2.23E−38 |
| Rdh16 | 1.13E−42 | 0.371605381 | 0.494 | 0.272 | 2.80E−38 |
| Ctage5 | 1.16E−42 | 0.321725387 | 0.837 | 0.683 | 2.88E−38 |
| Orm1 | 1.31E−42 | −0.316805138 | 0.921 | 0.952 | 3.25E−38 |
| Brap | 1.56E−42 | 0.311271816 | 0.642 | 0.427 | 3.86E−38 |
| Ptms | 7.41E−42 | 0.324316442 | 0.806 | 0.689 | 1.84E−37 |
| Acot12 | 1.09E−41 | 0.295700709 | 0.65 | 0.425 | 2.71E−37 |
| Bche | 1.31E−41 | 0.328717474 | 0.588 | 0.369 | 3.26E−37 |
| Reep6 | 1.56E−41 | 0.319298739 | 0.891 | 0.794 | 3.87E−37 |
| Sdhc | 2.99E−41 | 0.318089739 | 0.76 | 0.63 | 7.41E−37 |
| Tsc22d3 | 4.68E−41 | 0.342532759 | 0.534 | 0.312 | 1.16E−36 |
| Slc25a20 | 5.32E−41 | 0.321484297 | 0.671 | 0.498 | 1.32E−36 |
| Gabarapl1 | 1.41E−40 | 0.298356288 | 0.881 | 0.779 | 3.51E−36 |
| Abcc3 | 1.58E−40 | 0.330441312 | 0.733 | 0.542 | 3.92E−36 |
| Herpud1 | 1.87E−40 | 0.333265347 | 0.9 | 0.803 | 4.64E−36 |
| Saa2 | 1.89E−40 | −0.410878051 | 0.021 | 0.222 | 4.69E−36 |
| Decr1 | 2.56E−40 | 0.32410256 | 0.808 | 0.694 | 6.37E−36 |
| Cyp2c29 | 3.17E−40 | 0.410041422 | 0.962 | 0.93 | 7.87E−36 |
| Acot1 | 3.54E−40 | 0.298047999 | 0.459 | 0.237 | 8.79E−36 |
| Snord13 | 3.63E−40 | 0.265918809 | 0.386 | 0.175 | 9.03E−36 |
| Eif5 | 3.95E−40 | 0.274902626 | 0.879 | 0.74 | 9.80E−36 |
| Por | 5.68E−40 | 0.333398957 | 0.858 | 0.675 | 1.41E−35 |
| Tmem106b | 9.20E−40 | 0.328514628 | 0.727 | 0.557 | 2.28E−35 |
| Keg1 | 1.10E−39 | −0.510801616 | 0.378 | 0.545 | 2.73E−35 |
| Dusp6 | 2.04E−39 | 0.324885631 | 0.476 | 0.261 | 5.06E−35 |
| Arl4a | 2.96E−39 | 0.291095527 | 0.517 | 0.294 | 7.36E−35 |
| Slc27a5 | 3.04E−39 | 0.256735017 | 0.958 | 0.893 | 7.54E−35 |
| Elovl5 | 3.57E−39 | 0.323186895 | 0.765 | 0.58 | 8.86E−35 |
| Cd36 | 4.53E−39 | 0.331236859 | 0.299 | 0.123 | 1.12E−34 |
| Pnp1a8 | 8.92E−39 | 0.308301577 | 0.723 | 0.544 | 2.21E−34 |
| 0610005C13Rik | 1.18E−38 | 0.29785727 | 0.816 | 0.653 | 2.92E−34 |
| Aqp9 | 2.73E−38 | 0.334613695 | 0.801 | 0.647 | 6.79E−34 |
| Bnip3 | 4.39E−38 | 0.344268713 | 0.716 | 0.55 | 1.09E−33 |
| Ppara | 4.46E−38 | 0.305514783 | 0.614 | 0.407 | 1.11E−33 |
| Cyp2c44 | 6.48E−38 | 0.325839576 | 0.828 | 0.698 | 1.61E−33 |
| Zfp36l1 | 6.59E−38 | 0.329804827 | 0.601 | 0.402 | 1.64E−33 |
| Comt | 1.09E−37 | −0.269092283 | 0.902 | 0.931 | 2.70E−33 |
| Aldh3a2 | 1.63E−37 | 0.493314482 | 0.687 | 0.529 | 4.05E−33 |
| Ildr2 | 3.24E−37 | 0.25094453 | 0.356 | 0.159 | 8.06E−33 |
| Man2a1 | 3.28E−37 | 0.332441099 | 0.737 | 0.583 | 8.15E−33 |
| Gpld1 | 4.00E−37 | 0.303852677 | 0.748 | 0.577 | 9.92E−33 |
| Hamp | 4.80E−37 | 0.339616236 | 0.92 | 0.884 | 1.19E−32 |
| Ddx3x | 4.94E−37 | 0.289927181 | 0.701 | 0.513 | 1.23E−32 |
| Uhrf1bp1l | 5.68E−37 | 0.255854149 | 0.415 | 0.207 | 1.41E−32 |
| Palmd | 6.66E−37 | 0.25838388 | 0.43 | 0.219 | 1.65E−32 |
| Serpinf1 | 9.62E−37 | 0.257223443 | 0.934 | 0.864 | 2.39E−32 |
| Cox6a1 | 1.26E−36 | 0.251744129 | 0.943 | 0.887 | 3.14E−32 |
| Pcbp2 | 1.53E−36 | 0.311286813 | 0.621 | 0.438 | 3.81E−32 |
| Tat | 2.43E−36 | 0.25292193 | 0.981 | 0.903 | 6.04E−32 |
| Actb | 2.77E−36 | −0.311680152 | 0.839 | 0.886 | 6.88E−32 |
| Hnf4a | 4.21E−36 | 0.282370809 | 0.858 | 0.743 | 1.05E−31 |
| Mttp | 8.85E−36 | 0.275504341 | 0.816 | 0.697 | 2.20E−31 |
| Prkd3 | 9.88E−36 | 0.30495006 | 0.682 | 0.49 | 2.45E−31 |
| Dpys | 1.26E−35 | 0.262645107 | 0.824 | 0.682 | 3.12E−31 |
| Plin5 | 1.41E−35 | 0.266317901 | 0.532 | 0.315 | 3.51E−31 |
| Hsd17b7 | 2.01E−35 | 0.305636008 | 0.56 | 0.353 | 4.99E−31 |
| Dhdh | 3.32E−35 | 0.283722648 | 0.734 | 0.583 | 8.24E−31 |
| Pten | 3.91E−35 | 0.302502242 | 0.64 | 0.458 | 9.71E−31 |
| Hsd17b10 | 7.23E−35 | 0.309165601 | 0.841 | 0.7 | 1.80E−30 |
| Pbld1 | 9.61E−35 | 0.281095523 | 0.723 | 0.565 | 2.39E−30 |
| Hpgd | 1.02E−34 | 0.339771108 | 0.588 | 0.394 | 2.53E−30 |
| Secisbp2l | 1.16E−34 | 0.278110109 | 0.529 | 0.317 | 2.87E−30 |
| Lpin2 | 1.38E−34 | 0.284091908 | 0.781 | 0.597 | 3.44E−30 |
| Dmgdh | 2.94E−34 | 0.263486277 | 0.89 | 0.764 | 7.31E−30 |
| Cox8a | 3.32E−34 | 0.308861116 | 0.737 | 0.621 | 8.24E−30 |
| Slc25a13 | 4.31E−34 | 0.257252303 | 0.786 | 0.655 | 1.07E−29 |
| Dennd5b | 5.86E−34 | 0.28707652 | 0.548 | 0.334 | 1.46E−29 |
| Ei24 | 6.16E−34 | 0.271840234 | 0.667 | 0.48 | 1.53E−29 |
| Nfe2l1 | 7.85E−34 | 0.290178531 | 0.508 | 0.303 | 1.95E−29 |
| Brd2 | 7.88E−34 | 0.310435276 | 0.601 | 0.408 | 1.96E−29 |
| Gsta2 | 8.87E−34 | 0.311098868 | 0.522 | 0.333 | 2.20E−29 |
| Oaz1 | 1.04E−33 | −0.38606735 | 0.643 | 0.75 | 2.57E−29 |
| Ugp2 | 1.31E−33 | 0.293401416 | 0.858 | 0.749 | 3.26E−29 |
| Rps2 | 1.54E−33 | −0.396600754 | 0.609 | 0.757 | 3.83E−29 |
| Cpox | 1.57E−33 | 0.332618644 | 0.634 | 0.45 | 3.89E−29 |
| D4Wsu53e | 1.67E−33 | 0.303195253 | 0.597 | 0.385 | 4.14E−29 |
| Ncl | 4.23E−33 | 0.280946095 | 0.731 | 0.567 | 1.05E−28 |
| Etfdh | 4.77E−33 | 0.280245636 | 0.811 | 0.7 | 1.18E−28 |
| Slc7a2 | 6.50E−33 | 0.295894626 | 0.843 | 0.735 | 1.61E−28 |
| Igfbp1 | 6.60E−33 | 0.311528043 | 0.826 | 0.717 | 1.64E−28 |
| Eif4g2 | 1.44E−32 | 0.283785981 | 0.812 | 0.678 | 3.58E−28 |
| Fn1 | 1.75E−32 | 0.271755563 | 0.917 | 0.821 | 4.34E−28 |
| Cbr1 | 1.75E−32 | 0.282855589 | 0.591 | 0.395 | 4.35E−28 |
| Cyp2c54 | 2.81E−32 | 0.304653347 | 0.86 | 0.759 | 6.98E−28 |
| Chp1 | 8.03E−32 | 0.268716501 | 0.687 | 0.515 | 1.99E−27 |
| G0s2 | 1.20E−31 | 0.294687604 | 0.525 | 0.33 | 2.98E−27 |
| Hamp2 | 1.29E−31 | 0.305192386 | 0.203 | 0.07 | 3.19E−27 |
| Slc25a47 | 1.92E−31 | 0.277696086 | 0.96 | 0.927 | 4.76E−27 |
| Abca6 | 2.14E−31 | 0.282291899 | 0.686 | 0.507 | 5.31E−27 |
| Gfra1 | 2.84E−31 | 0.257142102 | 0.687 | 0.498 | 7.06E−27 |
| Ces3b | 3.07E−31 | −0.3398386 | 0.685 | 0.787 | 7.61E−27 |
| Gclc | 3.30E−31 | 0.270711809 | 0.839 | 0.718 | 8.19E−27 |
| Lpgat1 | 5.34E−31 | 0.274988157 | 0.671 | 0.502 | 1.33E−26 |
| Serpina3c | 1.26E−30 | −0.427440346 | 0.209 | 0.385 | 3.12E−26 |
| Hadh | 1.40E−30 | 0.26259162 | 0.774 | 0.642 | 3.47E−26 |
| Gm2a | 1.47E−30 | 0.316388243 | 0.587 | 0.427 | 3.66E−26 |
| Tomm70a | 1.62E−30 | 0.267009116 | 0.67 | 0.494 | 4.03E−26 |
| Acad11 | 1.64E−30 | 0.257789155 | 0.711 | 0.564 | 4.08E−26 |
| Csde1 | 1.66E−30 | 0.256715528 | 0.742 | 0.577 | 4.12E−26 |
| Atxn7l3b | 2.35E−30 | 0.282811183 | 0.654 | 0.476 | 5.85E−26 |
| Pxmp4 | 3.17E−30 | 0.27943845 | 0.687 | 0.526 | 7.88E−26 |
| Slc25a10 | 4.53E−30 | 0.250772586 | 0.701 | 0.536 | 1.12E−25 |
| Nucks1 | 5.40E−30 | 0.250923367 | 0.621 | 0.43 | 1.34E−25 |
| Erbb2ip | 5.61E−30 | 0.255253663 | 0.561 | 0.364 | 1.39E−25 |
| BC005537 | 5.73E−30 | 0.286873064 | 0.611 | 0.45 | 1.42E−25 |
| Aldh6a1 | 7.19E−30 | 0.266896818 | 0.829 | 0.704 | 1.78E−25 |
| Gstm3 | 7.39E−30 | −0.566844134 | 0.23 | 0.414 | 1.83E−25 |
| Cd1d1 | 7.83E−30 | 0.269180487 | 0.747 | 0.612 | 1.95E−25 |
| Hsd3b7 | 1.45E−29 | −0.328539206 | 0.729 | 0.798 | 3.61E−25 |
| Ppib | 1.51E−29 | −0.35186504 | 0.696 | 0.767 | 3.75E−25 |
| Pigr | 2.93E−29 | 0.275356693 | 0.957 | 0.947 | 7.29E−25 |
| Baat | 3.16E−29 | 0.262488362 | 0.862 | 0.767 | 7.84E−25 |
| Pdk1 | 3.33E−29 | 0.256134304 | 0.438 | 0.251 | 8.28E−25 |
| Atf5 | 6.07E−29 | 0.27075899 | 0.745 | 0.641 | 1.51E−24 |
| Mup1 | 6.60E−29 | −0.613763846 | 0.419 | 0.583 | 1.64E−24 |
| Pipox | 8.09E−29 | 0.267912272 | 0.69 | 0.549 | 2.01E−24 |
| Sparcl1 | 1.50E−28 | −0.253616986 | 0 | 0.136 | 3.72E−24 |
| Sc4mol | 2.00E−28 | 0.281384033 | 0.781 | 0.642 | 4.97E−24 |
| Ptplad1 | 2.88E−28 | 0.278296625 | 0.654 | 0.496 | 7.14E−24 |
| C8a | 5.17E−28 | −0.294138173 | 0.867 | 0.887 | 1.28E−23 |
| Lrrc58 | 6.92E−28 | 0.264060967 | 0.543 | 0.359 | 1.72E−23 |
| Actn4 | 7.75E−28 | 0.260860094 | 0.6 | 0.425 | 1.92E−23 |
| Nfib | 1.61E−27 | 0.250093732 | 0.55 | 0.364 | 4.00E−23 |
| Maob | 2.53E−27 | 0.272235996 | 0.708 | 0.575 | 6.28E−23 |
| Apol7a | 4.08E−27 | 0.271621562 | 0.559 | 0.382 | 1.01E−22 |
| Ces1e | 5.81E−27 | 0.294372509 | 0.71 | 0.576 | 1.44E−22 |
| Hmgcs1 | 7.34E−27 | 0.296651807 | 0.858 | 0.703 | 1.82E−22 |
| Fmo1 | 8.35E−27 | 0.263993508 | 0.606 | 0.415 | 2.07E−22 |
| Ppm1a | 1.41E−26 | 0.275992744 | 0.571 | 0.39 | 3.49E−22 |
| Ptp4a2 | 1.73E−26 | 0.258986528 | 0.691 | 0.547 | 4.29E−22 |
| Sh3glb1 | 2.26E−26 | 0.253791572 | 0.433 | 0.258 | 5.62E−22 |
| Ttc39c | 2.27E−26 | −0.350463351 | 0.546 | 0.642 | 5.63E−22 |
| Gm4076 | 3.58E−26 | −0.301057344 | 0.035 | 0.184 | 8.88E−22 |
| Uroc1 | 3.89E−26 | 0.307355817 | 0.726 | 0.615 | 9.66E−22 |
| Mt2 | 9.21E−26 | 0.313597451 | 0.548 | 0.365 | 2.29E−21 |
| Pdia6 | 1.14E−25 | −0.335875757 | 0.611 | 0.712 | 2.82E−21 |
| Iqgap2 | 3.16E−25 | 0.264494476 | 0.799 | 0.677 | 7.85E−21 |
| Ptprd | 6.32E−25 | 0.271860854 | 0.582 | 0.42 | 1.57E−20 |
| 0610007P14Rik | 8.28E−25 | 0.251927159 | 0.512 | 0.346 | 2.06E−20 |
| Ubr3 | 2.90E−24 | 0.252873563 | 0.585 | 0.41 | 7.20E−20 |
| Mup13 | 6.00E−24 | −0.469138263 | 0.418 | 0.546 | 1.49E−19 |
| Upp2 | 1.76E−23 | −0.285897303 | 0.545 | 0.673 | 4.36E−19 |
| 8430408G22Rik | 2.02E−23 | 0.252689739 | 0.559 | 0.392 | 5.03E−19 |
| Ppap2b | 2.66E−23 | 0.257285533 | 0.593 | 0.43 | 6.61E−19 |
| Cfhr2 | 6.41E−23 | −0.303339186 | 0.885 | 0.868 | 1.59E−18 |
| Hsd17b2 | 1.28E−22 | −0.300549533 | 0.614 | 0.711 | 3.19E−18 |
| Ifitm3 | 1.30E−22 | −0.279693974 | 0.742 | 0.812 | 3.22E−18 |
| Atp2a2 | 2.79E−22 | 0.253693334 | 0.64 | 0.495 | 6.92E−18 |
| Mup2 | 1.19E−20 | −0.500849516 | 0.256 | 0.392 | 2.96E−16 |
| Hsd3b3 | 1.32E−20 | −0.313033598 | 0.499 | 0.605 | 3.27E−16 |
| Pnpla7 | 1.51E−20 | −0.339749669 | 0.455 | 0.572 | 3.74E−16 |
| Serp1 | 2.63E−20 | −0.299204955 | 0.671 | 0.721 | 6.53E−16 |
| Naa50 | 2.84E−20 | 0.265808764 | 0.517 | 0.358 | 7.04E−16 |
| Eef1b2 | 4.19E−20 | −0.268563341 | 0.743 | 0.78 | 1.04E−15 |
| Cyp2e1 | 6.55E−20 | 0.347156447 | 0.93 | 0.911 | 1.63E−15 |
| Fos | 8.58E−20 | 0.327248556 | 0.619 | 0.51 | 2.13E−15 |
| Cyp2c70 | 2.06E−19 | −0.278293267 | 0.828 | 0.834 | 5.10E−15 |
| Prdx4 | 6.87E−19 | −0.313999267 | 0.517 | 0.607 | 1.71E−14 |
| Rarres1 | 1.14E−18 | −0.287428253 | 0.274 | 0.42 | 2.83E−14 |
| Cyp7a1 | 3.08E−18 | 0.274384618 | 0.398 | 0.259 | 7.66E−14 |
| Csad | 4.37E−18 | 0.290595078 | 0.843 | 0.744 | 1.09E−13 |
| Etnppl | 4.63E−18 | 0.407010453 | 0.677 | 0.606 | 1.15E−13 |
| Malat1 | 2.80E−17 | −0.464666369 | 0.702 | 0.78 | 6.96E−13 |
| Mup4 | 3.09E−17 | −0.693444431 | 0.824 | 0.898 | 7.68E−13 |
| Psmb1 | 3.10E−17 | −0.265941959 | 0.703 | 0.759 | 7.70E−13 |
| Rgn | 3.36E−17 | 0.261419562 | 0.899 | 0.874 | 8.33E−13 |
| Ndufb11 | 4.01E−17 | −0.283194323 | 0.669 | 0.735 | 9.96E−13 |
| Krt8 | 8.75E−16 | −0.265010151 | 0.47 | 0.578 | 2.17E−11 |
| Rpl32 | 3.29E−14 | −0.263090496 | 0.689 | 0.757 | 8.17E−10 |
| Cyp2c37 | 9.91E−14 | 0.289653212 | 0.71 | 0.608 | 2.46E−09 |
| Selk | 1.39E−13 | −0.264704833 | 0.393 | 0.486 | 3.45E−09 |
| Psmb5 | 2.01E−13 | −0.263312926 | 0.241 | 0.348 | 4.98E−09 |
| Ifi27 | 3.66E−13 | −0.25720865 | 0.325 | 0.453 | 9.08E−09 |
| Nme1 | 3.82E−13 | −0.272417976 | 0.561 | 0.622 | 9.49E−09 |
| Hes6 | 4.04E−13 | −0.278057796 | 0.33 | 0.42 | 1.00E−08 |
| Rps8 | 1.24E−12 | −0.258468642 | 0.378 | 0.471 | 3.08E−08 |
| Ubb | 3.02E−08 | −0.268532383 | 0.379 | 0.45 | 0.000750415 |
| Rps15 | 5.07E−08 | −0.254504371 | 0.468 | 0.528 | 0.001260068 |
| Hsd17b13 | 5.79E−07 | 0.267753182 | 0.755 | 0.74 | 0.014383233 |
| mmu-mir-6236 | 1.15E−06 | −0.627231332 | 0.801 | 0.837 | 0.028554582 |
| Oat | 2.03E−05 | 0.343909058 | 0.653 | 0.611 | 0.504812214 |
| Ftl1 | 2.40E−05 | −0.279770666 | 0.363 | 0.409 | 0.597068016 |
| mt-Atp6 | 0.000145832 | −0.39551085 | 0.43 | 0.452 | 1 |
| Gm20594 | 0.001254387 | −0.292751004 | 0.262 | 0.294 | 1 |
| Rpl41 | 0.062445139 | −0.2575003 | 0.274 | 0.278 | 1 |

TABLE 5a
Differentially expressed genes between time point PHX3 FC.1 and untreated (UT).

| | p_val | avg_logFC | pct.1 | pct.2 | p_val_adj |
|---|---|---|---|---|---|
| Saa2 | 0 | 2.971862499 | 0.999 | 0.222 | 0 |
| Mt1 | 0 | 2.610854994 | 1 | 0.556 | 0 |
| Mt2 | 0 | 2.6060431 | 1 | 0.365 | 0 |
| Saa1 | 0 | 2.202156726 | 1 | 0.662 | 0 |
| Steap4 | 0 | 1.890015167 | 0.973 | 0.222 | 0 |
| Serpina3n | 0 | 1.759758939 | 0.992 | 0.574 | 0 |
| Lcn2 | 0 | 1.499141183 | 0.815 | 0.07 | 0 |
| Lpin1 | 0 | 1.327657625 | 0.94 | 0.31 | 0 |
| n-R5-8s1 | 0 | 1.289598504 | 0.805 | 0.031 | 0 |
| Igfbp1 | 0 | 1.288789902 | 0.99 | 0.717 | 0 |
| Cxcl1 | 0 | 1.230398019 | 0.919 | 0.314 | 0 |
| Scd1 | 0 | 1.081676999 | 0.998 | 0.901 | 0 |
| Gm26924 | 0 | 1.063463323 | 1 | 1 | 0 |
| Il1r1 | 0 | 0.998168131 | 0.8 | 0.167 | 0 |
| Fga | 0 | 0.981249724 | 1 | 0.997 | 0 |
| Gm24601 | 0 | 0.924568273 | 0.661 | 0.015 | 0 |
| Nnmt | 0 | 0.901052904 | 0.97 | 0.716 | 0 |
| Itih4 | 0 | 0.887186241 | 0.999 | 0.948 | 0 |
| St3gal5 | 0 | 0.882761517 | 0.915 | 0.49 | 0 |
| Tacc2 | 0 | 0.877623973 | 0.707 | 0.071 | 0 |
| Lrg1 | 0 | 0.870250525 | 0.981 | 0.736 | 0 |
| Fam134b | 0 | 0.865578177 | 0.876 | 0.405 | 0 |
| Tat | 0 | 0.857965136 | 0.998 | 0.903 | 0 |
| Saa4 | 0 | 0.854921603 | 0.981 | 0.749 | 0 |
| Thrsp | 0 | 0.836598219 | 0.98 | 0.627 | 0 |
| Slc39a14 | 0 | 0.831584936 | 0.849 | 0.343 | 0 |
| Lars2 | 0 | 0.816795688 | 1 | 0.994 | 0 |
| Fgg | 0 | 0.809322713 | 1 | 0.991 | 0 |
| Slc38a2 | 0 | 0.797899442 | 0.874 | 0.413 | 0 |
| Serpina3m | 0 | 0.779052745 | 0.961 | 0.722 | 0 |
| Hp | 0 | 0.774785279 | 1 | 0.999 | 0 |
| Fgb | 0 | 0.739519125 | 1 | 1 | 0 |
| Gm15564 | 0 | 0.735157137 | 0.999 | 0.956 | 0 |
| Serpina10 | 0 | 0.733655099 | 0.934 | 0.641 | 0 |
| Plin2 | 0 | 0.720463076 | 0.952 | 0.714 | 0 |
| Aass | 0 | 0.705615345 | 0.965 | 0.716 | 0 |
| Fmo5 | 0 | 0.690160801 | 0.991 | 0.837 | 0 |
| Fn1 | 0 | 0.684266092 | 0.983 | 0.821 | 0 |
| Fkbp5 | 0 | 0.663304836 | 0.665 | 0.117 | 0 |
| Slc25a47 | 0 | 0.635928777 | 0.994 | 0.927 | 0 |
| Mat1a | 0 | 0.623734998 | 0.999 | 0.966 | 0 |
| Aldob | 0 | 0.490200848 | 1 | 0.996 | 0 |
| Mup3 | 0 | −0.526410044 | 1 | 1 | 0 |
| Cyb5 | 0 | −0.71965743 | 0.992 | 0.983 | 0 |
| mt-Rnr1 | 0 | −0.872186844 | 1 | 1 | 0 |
| mt-Rnr2 | 0 | −0.873557854 | 1 | 1 | 0 |
| Mup11 | 0 | −0.976755523 | 0.996 | 1 | 0 |
| mt-Nd2 | 0 | −1.128776891 | 0.996 | 0.999 | 0 |
| mt-Nd1 | 0 | −1.136736863 | 0.999 | 1 | 0 |
| mt-Nd5 | 0 | −1.145550707 | 0.974 | 0.995 | 0 |
| mt-Co3 | 0 | −1.233961828 | 0.106 | 0.603 | 0 |
| Gm13775 | 0 | −1.255657303 | 0.205 | 0.761 | 0 |
| mt-Cytb | 0 | −1.296043506 | 0.993 | 0.999 | 0 |
| mt-Co2 | 0 | −1.341686736 | 0.1 | 0.629 | 0 |
| mt-Co1 | 0 | −1.381307687 | 0.986 | 0.999 | 0 |
| mt-Nd4 | 0 | −1.630375247 | 0.977 | 0.998 | 0 |
| Mup4 | 0 | −1.805301681 | 0.374 | 0.898 | 0 |
| Mup6 | 0 | −2.386597202 | 0.714 | 0.992 | 0 |
| Mup9 | 0 | −2.453596744 | 0.401 | 0.976 | 0 |
| Mup17 | 0 | −2.514472885 | 0.899 | 1 | 0 |
| Mup18 | 0 | −2.617739049 | 0.547 | 0.997 | 0 |
| Mup19 | 0 | −2.932276479 | 0.84 | 1 | 0 |
| Acox1 | 2.96439387504748e−323 | 0.540022158 | 0.998 | 0.966 | 7.3611828705179e−319 |
| Hnf4a | 1.23516411460312e−322 | 0.644195253 | 0.969 | 0.743 | 3.06715952938246e−318 |
| B3galt1 | 2.95451256213065e−321 | 0.788734938 | 0.634 | 0.163 | 7.33664559428284e−317 |
| Cyp2b10 | 3.42189866309647e−320 | −1.254972098 | 0.132 | 0.542 | 8.49725876020116e−316 |
| Mup5 | 5.13739339858645e−319 | −1.484560547 | 0.52 | 0.839 | 1.27571752873699e−314 |
| H3f3b | 1.54603021896643e−318 | 0.755567107 | 0.831 | 0.419 | 3.83910223973744e−314 |
| Btg2 | 0.00E+00 | 0.772686832 | 0.708 | 0.241 | 2.89E−305 |
| Sephs2 | 0.00E+00 | 0.618707003 | 0.974 | 0.761 | 3.26E−305 |
| Ankrd55 | 1.93E−296 | −1.464696038 | 0.374 | 0.696 | 4.78E−292 |
| Hspb8 | 2.89E−295 | 0.667847253 | 0.914 | 0.597 | 7.19E−291 |
| Slc7a2 | 8.07E−288 | 0.622326529 | 0.964 | 0.735 | 2.01E−283 |
| Cldn14 | 1.24E−283 | 0.511583526 | 0.505 | 0.076 | 3.09E−279 |
| C3 | 8.73E−282 | 0.482674202 | 0.999 | 0.99 | 2.17E−277 |
| Gjb2 | 1.09E−277 | 0.639507436 | 0.901 | 0.572 | 2.70E−273 |
| Rcan1 | 1.31E−277 | 0.509239866 | 0.522 | 0.085 | 3.25E−273 |
| Errfi1 | 1.31E−267 | 0.500850526 | 0.998 | 0.911 | 3.25E−263 |
| Pfkfb3 | 1.05E−265 | 0.426580733 | 0.421 | 0.039 | 2.60E−261 |
| Arhgef26 | 1.83E−264 | 0.568544702 | 0.67 | 0.203 | 4.56E−260 |
| Cyp2c29 | 3.96E−262 | −1.027267705 | 0.751 | 0.93 | 9.83E−258 |
| Egfr | 1.26E−254 | 0.555834247 | 0.989 | 0.809 | 3.12E−250 |
| Gsta3 | 1.87E−250 | −0.53784222 | 0.993 | 0.956 | 4.64E−246 |
| Bcl3 | 1.73E−248 | 0.419650143 | 0.479 | 0.074 | 4.30E−244 |
| Snx10 | 3.96E−247 | 0.558244633 | 0.63 | 0.188 | 9.84E−243 |
| Pck1 | 1.36E−246 | 0.43447474 | 0.999 | 0.987 | 3.37E−242 |
| Mup16 | 4.33E−246 | −0.848726713 | 0.816 | 0.934 | 1.07E−241 |
| Bach1 | 1.17E−243 | 0.533237374 | 0.589 | 0.151 | 2.91E−239 |
| Tifa | 8.59E−243 | 0.392339477 | 0.393 | 0.036 | 2.13E−238 |
| Hpx | 1.42E−241 | 0.460272423 | 1 | 0.997 | 3.52E−237 |
| Ugt2b5 | 1.30E−240 | −0.423085187 | 0.991 | 0.959 | 3.23E−236 |
| Etnk2 | 3.72E−240 | 0.592353201 | 0.918 | 0.655 | 9.25E−236 |
| 2010003K11Rik | 8.42E−239 | 0.372219971 | 0.412 | 0.046 | 2.09E−234 |
| Rapgef4 | 1.08E−237 | 0.588317735 | 0.836 | 0.436 | 2.69E−233 |
| Tiparp | 5.34E−233 | 0.489122153 | 0.485 | 0.087 | 1.33E−228 |
| Gpt2 | 4.65E−230 | 0.533331785 | 0.949 | 0.725 | 1.15E−225 |
| Slc25a25 | 1.00E−226 | 0.544465891 | 0.945 | 0.704 | 2.48E−222 |
| Mup10 | 7.92E−224 | 0.428268029 | 0.965 | 0.692 | 1.97E−219 |
| Txnip | 3.60E−223 | 0.552612237 | 0.741 | 0.297 | 8.94E−219 |
| Cyp2c50 | 1.21E−219 | −0.763912176 | 0.693 | 0.888 | 3.02E−215 |
| Xbp1 | 4.00E−219 | 0.539986 | 0.89 | 0.57 | 9.93E−215 |
| Upp2 | 6.55E−218 | 0.541753512 | 0.934 | 0.673 | 1.63E−213 |
| Mfsd2a | 1.62E−217 | 0.496351884 | 0.563 | 0.148 | 4.02E−213 |
| Ephb3 | 4.02E−217 | 0.188403248 | 0.244 | 0.001 | 9.97E−213 |
| Actb | 2.92E−216 | 0.456981443 | 0.99 | 0.886 | 7.25E−212 |
| mmu-mir-6236 | 7.87E−215 | 0.468866739 | 0.989 | 0.837 | 1.95E−210 |
| Fbf1 | 1.44E−212 | 0.298573909 | 0.336 | 0.027 | 3.58E−208 |
| Trib1 | 3.58E−209 | 0.581754281 | 0.705 | 0.308 | 8.89E−205 |
| Hsd3b5 | 4.49E−209 | −0.587528886 | 0.49 | 0.587 | 1.12E−204 |
| Arg1 | 8.68E−205 | 0.381616351 | 1 | 0.982 | 2.16E−200 |
| Plin5 | 3.05E−204 | 0.489257529 | 0.736 | 0.315 | 7.56E−200 |
| Plk3 | 5.05E−204 | 0.488016367 | 0.644 | 0.226 | 1.26E−199 |
| Mup13 | 2.17E−203 | −0.755608269 | 0.303 | 0.546 | 5.38E−199 |
| Mtss1 | 7.69E−203 | 0.552137981 | 0.812 | 0.454 | 1.91E−198 |
| Klf6 | 1.10E−202 | 0.3616937 | 0.346 | 0.034 | 2.74E−198 |
| Itih3 | 1.01E−201 | 0.469631651 | 0.988 | 0.89 | 2.51E−197 |
| Gm23935 | 3.69E−201 | 0.498066925 | 0.999 | 0.925 | 9.17E−197 |
| Mup21 | 3.26E−199 | −0.668770594 | 0.829 | 0.929 | 8.09E−195 |
| Adck3 | 1.22E−198 | −0.482940158 | 0.729 | 0.764 | 3.02E−194 |
| Cyp2c54 | 5.69E−198 | −0.712859584 | 0.491 | 0.759 | 1.41E−193 |
| Slc10a2 | 2.81E−196 | 0.30563824 | 0.376 | 0.05 | 6.99E−192 |
| Egr1 | 4.80E−196 | 0.620480469 | 0.79 | 0.447 | 1.19E−191 |
| Sdc4 | 5.23E−196 | 0.43560504 | 0.986 | 0.867 | 1.30E−191 |
| Serpina3c | 5.27E−196 | −0.554657351 | 0.118 | 0.385 | 1.31E−191 |
| Apcs | 5.83E−195 | 0.573439593 | 0.854 | 0.525 | 1.45E−190 |
| Atf3 | 1.04E−193 | 0.597274889 | 0.56 | 0.179 | 2.59E−189 |
| Hsd3b7 | 2.55E−191 | −0.451728474 | 0.795 | 0.798 | 6.34E−187 |
| Orm1 | 4.40E−190 | 0.463411533 | 0.993 | 0.952 | 1.09E−185 |
| Glul | 9.28E−190 | 0.478908893 | 0.58 | 0.18 | 2.30E−185 |
| Pzp | 1.11E−189 | 0.387351113 | 0.999 | 0.986 | 2.75E−185 |
| Prg4 | 7.24E−189 | 0.480504574 | 0.585 | 0.188 | 1.80E−184 |
| Fgl1 | 2.00E−188 | 0.49338252 | 0.961 | 0.784 | 4.97E−184 |
| Gpcpd1 | 4.07E−188 | 0.53780646 | 0.778 | 0.398 | 1.01E−183 |
| Klf10 | 5.00E−188 | 0.304779554 | 0.388 | 0.06 | 1.24E−183 |
| Rgs16 | 2.31E−187 | 0.438105488 | 0.482 | 0.113 | 5.72E−183 |
| Ptp4a1 | 3.32E−187 | 0.393137367 | 0.528 | 0.145 | 8.25E−183 |
| mt-Atp6 | 3.88E−186 | −0.852555757 | 0.138 | 0.452 | 9.63E−182 |
| Fam35a | 2.55E−184 | 0.470461753 | 0.642 | 0.246 | 6.34E−180 |
| Rhob | 2.99E−184 | 0.37207281 | 0.4 | 0.069 | 7.42E−180 |
| Mup15 | 8.65E−184 | −1.074409915 | 0.357 | 0.617 | 2.15E−179 |
| Cyp3a25 | 1.35E−183 | −0.41051207 | 0.928 | 0.919 | 3.35E−179 |
| Got1 | 2.81E−183 | 0.542891511 | 0.866 | 0.587 | 6.99E−179 |
| Ces1d | 1.16E−179 | −0.385155368 | 0.944 | 0.923 | 2.88E−175 |
| Shb | 1.22E−178 | 0.430613558 | 0.589 | 0.204 | 3.02E−174 |
| Sds | 6.42E−177 | 0.787094904 | 0.868 | 0.67 | 1.59E−172 |
| Abca1 | 4.57E−176 | 0.50506234 | 0.756 | 0.377 | 1.14E−171 |
| 1810055G02Rik | 7.51E−174 | 0.382584093 | 0.528 | 0.155 | 1.86E−169 |
| Stat3 | 1.08E−172 | 0.460515229 | 0.701 | 0.318 | 2.69E−168 |
| Akr1c6 | 4.37E−171 | −0.43492211 | 0.992 | 0.971 | 1.08E−166 |
| Apoe | 2.24E−170 | −0.26209828 | 1 | 1 | 5.55E−166 |
| Igfbp4 | 5.75E−170 | 0.387861679 | 0.986 | 0.866 | 1.43E−165 |
| Cdkn1a | 2.80E−169 | 0.449325471 | 0.572 | 0.195 | 6.96E−165 |
| Gfra1 | 6.66E−169 | 0.474694426 | 0.835 | 0.498 | 1.65E−164 |
| Gc | 2.58E−168 | 0.233764264 | 1 | 0.999 | 6.42E−164 |
| Nrg4 | 3.51E−168 | 0.246089001 | 0.306 | 0.034 | 8.72E−164 |
| Ivns1abp | 1.57E−166 | 0.46254094 | 0.688 | 0.315 | 3.89E−162 |
| Fh1 | 2.76E−165 | 0.471428771 | 0.915 | 0.648 | 6.86E−161 |
| Myo1e | 9.09E−164 | 0.399456672 | 0.551 | 0.182 | 2.26E−159 |
| Slc41a2 | 1.23E−163 | 0.3685439 | 0.482 | 0.134 | 3.04E−159 |
| Mup8 | 2.63E−163 | −0.793731191 | 0.111 | 0.373 | 6.53E−159 |
| Slc25a51 | 3.37E−163 | 0.372157664 | 0.522 | 0.161 | 8.37E−159 |
| Psen2 | 3.10E−162 | −0.419555606 | 0.749 | 0.774 | 7.69E−158 |
| Dak | 4.15E−162 | 0.481947664 | 0.867 | 0.563 | 1.03E−157 |
| Apoc4 | 4.45E−160 | −0.356086407 | 0.999 | 0.98 | 1.10E−155 |
| Arid5a | 1.22E−159 | 0.163384737 | 0.208 | 0.005 | 3.03E−155 |
| Fasn | 1.97E−159 | 0.467168076 | 0.719 | 0.345 | 4.89E−155 |
| Bhlhe40 | 9.52E−159 | 0.403249298 | 0.628 | 0.251 | 2.36E−154 |
| Tmem87b | 4.61E−156 | 0.288330587 | 0.415 | 0.096 | 1.15E−151 |
| Ddx3x | 2.02E−155 | 0.47100162 | 0.823 | 0.513 | 5.01E−151 |
| Ttr | 3.46E−155 | 0.248320061 | 1 | 1 | 8.59E−151 |
| Aldh1a1 | 9.02E−152 | −0.40245097 | 0.984 | 0.956 | 2.24E−147 |
| Slc25a22 | 2.27E−151 | 0.390931532 | 0.649 | 0.282 | 5.65E−147 |
| Rnf103 | 9.68E−150 | 0.438540394 | 0.701 | 0.35 | 2.40E−145 |
| Scara5 | 1.84E−149 | 0.221632911 | 0.324 | 0.054 | 4.57E−145 |
| B4galt1 | 3.68E−147 | 0.321380868 | 0.494 | 0.155 | 9.13E−143 |
| Hacl1 | 1.79E−146 | −0.332899987 | 0.842 | 0.797 | 4.44E−142 |
| Rnf125 | 2.78E−146 | −0.463320018 | 0.312 | 0.493 | 6.90E−142 |
| Gm26917 | 2.01E−145 | 0.46739621 | 0.523 | 0.181 | 4.98E−141 |
| Junb | 2.83E−145 | 0.458500682 | 0.788 | 0.47 | 7.03E−141 |
| Cps1 | 5.10E−145 | 0.336429448 | 0.999 | 0.971 | 1.27E−140 |
| Atp1b1 | 2.13E−144 | 0.424801231 | 0.567 | 0.226 | 5.28E−140 |
| Glud1 | 8.86E−144 | 0.341724783 | 0.991 | 0.905 | 2.20E−139 |
| D10Wsu102e | 2.68E−142 | 0.256022156 | 0.338 | 0.063 | 6.64E−138 |
| Hyou1 | 3.20E−142 | 0.431109873 | 0.731 | 0.385 | 7.93E−138 |
| Jmjd1c | 3.24E−142 | 0.39612601 | 0.516 | 0.178 | 8.04E−138 |
| 4933426M11Rik | 8.78E−142 | 0.28624304 | 0.419 | 0.109 | 2.18E−137 |
| Apoa5 | 9.82E−142 | 0.30259839 | 0.999 | 0.974 | 2.44E−137 |
| Scp2 | 5.80E−141 | 0.279911381 | 0.997 | 0.986 | 1.44E−136 |
| Jun | 2.39E−140 | 0.479246114 | 0.864 | 0.593 | 5.93E−136 |
| Cyp2a5 | 4.56E−140 | −0.938183249 | 0.278 | 0.555 | 1.13E−135 |
| Mup12 | 4.71E−140 | −0.589618573 | 0.792 | 0.86 | 1.17E−135 |
| Cpt1a | 8.94E−140 | 0.42860734 | 0.894 | 0.651 | 2.22E−135 |
| Pik3r1 | 1.33E−139 | 0.389193614 | 0.577 | 0.231 | 3.30E−135 |
| Apob | 5.29E−139 | 0.416824707 | 0.997 | 0.989 | 1.31E−134 |
| Hmox1 | 1.29E−137 | 0.169723562 | 0.22 | 0.016 | 3.21E−133 |
| Epas1 | 1.76E−136 | 0.38425941 | 0.646 | 0.297 | 4.38E−132 |
| Ugt2b36 | 9.83E−136 | −0.287193139 | 0.978 | 0.93 | 2.44E−131 |
| Mbd1 | 2.08E−135 | 0.348094524 | 0.507 | 0.176 | 5.16E−131 |
| Mup2 | 8.39E−135 | −0.650107006 | 0.161 | 0.392 | 2.08E−130 |
| Pik3ap1 | 9.99E−135 | 0.19440062 | 0.283 | 0.043 | 2.48E−130 |
| Foxq1 | 5.85E−134 | 0.22551931 | 0.377 | 0.096 | 1.45E−129 |
| Slc22a5 | 9.36E−134 | 0.185456618 | 0.32 | 0.065 | 2.32E−129 |
| Gadd45g | 2.40E−133 | 0.392031882 | 0.899 | 0.639 | 5.96E−129 |
| Bcl2l1 | 6.34E−133 | 0.280315334 | 0.426 | 0.122 | 1.57E−128 |
| Fos | 6.58E−133 | 0.524733327 | 0.785 | 0.51 | 1.63E−128 |
| Ptpn1 | 8.98E−133 | 0.268263225 | 0.402 | 0.107 | 2.23E−128 |
| Slc25a15 | 2.35E−132 | 0.402049896 | 0.931 | 0.738 | 5.83E−128 |
| Tdo2 | 9.76E−132 | 0.25156007 | 0.999 | 0.991 | 2.42E−127 |
| Rnd3 | 2.07E−131 | 0.285147682 | 0.375 | 0.091 | 5.13E−127 |
| Adh1 | 3.50E−131 | −0.295980789 | 0.995 | 0.96 | 8.69E−127 |
| Litaf | 6.42E−131 | 0.396007707 | 0.603 | 0.278 | 1.59E−126 |
| Gm5096 | 1.40E−130 | −0.399548106 | 0.364 | 0.468 | 3.47E−126 |
| Wbp1l | 9.53E−130 | 0.409138156 | 0.867 | 0.606 | 2.37E−125 |
| Grn | 5.30E−129 | 0.403315761 | 0.726 | 0.396 | 1.32E−124 |
| Tns1 | 6.02E−129 | 0.33981795 | 0.524 | 0.196 | 1.50E−124 |
| Cyp2c37 | 7.90E−129 | −0.662335734 | 0.314 | 0.608 | 1.96E−124 |
| Ahcy | 1.61E−128 | −0.375627724 | 0.918 | 0.916 | 4.01E−124 |
| Foxa3 | 4.51E−128 | 0.307992015 | 0.505 | 0.182 | 1.12E−123 |
| Qsox1 | 7.12E−128 | 0.378486857 | 0.925 | 0.707 | 1.77E−123 |
| Cth | 1.21E−127 | −0.308847665 | 0.898 | 0.838 | 3.00E−123 |
| Id2 | 3.26E−127 | 0.430147785 | 0.826 | 0.579 | 8.09E−123 |
| 1700017B05Rik | 7.08E−127 | 0.182766029 | 0.263 | 0.037 | 1.76E−122 |
| Myh9 | 9.38E−127 | 0.399128031 | 0.653 | 0.318 | 2.33E−122 |
| Cox6c | 7.06E−126 | −0.41128283 | 0.893 | 0.884 | 1.75E−121 |
| Raph1 | 3.03E−125 | 0.242670968 | 0.382 | 0.1 | 7.52E−121 |
| Ier2 | 5.55E−123 | 0.452598264 | 0.763 | 0.49 | 1.38E−118 |
| Cfh | 6.71E−123 | 0.312615767 | 0.991 | 0.897 | 1.67E−118 |
| Itih2 | 1.73E−122 | 0.336714211 | 0.984 | 0.884 | 4.30E−118 |
| Ccnl1 | 1.52E−121 | 0.341338448 | 0.526 | 0.206 | 3.77E−117 |
| Rsp14 | 1.65E−121 | −0.346340977 | 0.936 | 0.917 | 4.10E−117 |
| Cyp8b1 | 2.36E−121 | −0.364571013 | 0.714 | 0.745 | 5.87E−117 |
| Cox4i1 | 2.43E−121 | −0.288035158 | 0.992 | 0.958 | 6.04E−117 |
| Eef1a1 | 6.76E−121 | 0.340768186 | 0.967 | 0.85 | 1.68E−116 |
| Nedd4l | 2.66E−120 | 0.271827098 | 0.447 | 0.147 | 6.62E−116 |
| Inmt | 6.92E−120 | −0.308364158 | 0.966 | 0.884 | 1.72E−115 |
| Slc37a1 | 1.40E−119 | 0.133887938 | 0.198 | 0.015 | 3.49E−115 |
| Cep85l | 1.73E−119 | 0.202664771 | 0.347 | 0.089 | 4.29E−115 |
| Elovl5 | 6.66E−119 | 0.382267188 | 0.863 | 0.58 | 1.65E−114 |
| Zbtb16 | 7.66E−119 | 0.232600907 | 0.414 | 0.131 | 1.90E−114 |
| Il17ra | 3.02E−118 | 0.203529938 | 0.302 | 0.061 | 7.51E−114 |
| Hip1r | 3.74E−118 | 0.213858215 | 0.362 | 0.098 | 9.27E−114 |
| Gcc1 | 5.57E−118 | 0.175053461 | 0.258 | 0.04 | 1.38E−113 |
| Plg | 5.78E−118 | 0.26619661 | 0.998 | 0.961 | 1.43E−113 |
| Eif4g2 | 8.12E−118 | 0.384302798 | 0.905 | 0.678 | 2.02E−113 |
| Atg14 | 1.14E−117 | 0.231648357 | 0.369 | 0.1 | 2.83E−113 |
| Ppip5k2 | 1.68E−117 | 0.30299148 | 0.51 | 0.197 | 4.16E−113 |
| Ulk1 | 3.53E−117 | 0.297841785 | 0.499 | 0.189 | 8.77E−113 |
| Elovl6 | 1.30E−116 | 0.37962641 | 0.553 | 0.24 | 3.24E−112 |
| Pon1 | 1.35E−116 | −0.302819466 | 0.936 | 0.906 | 3.35E−112 |
| Il6ra | 6.12E−116 | 0.251696836 | 0.431 | 0.142 | 1.52E−111 |
| Adamts1 | 3.82E−115 | 0.15752777 | 0.202 | 0.018 | 9.49E−111 |
| Ndufb11 | 7.95E−115 | −0.36792318 | 0.722 | 0.735 | 1.97E−110 |
| Ets2 | 1.01E−113 | 0.183062901 | 0.285 | 0.056 | 2.50E−109 |
| Igfbp2 | 4.23E−113 | −0.348403589 | 0.886 | 0.885 | 1.05E−108 |
| Fads2 | 1.77E−112 | 0.323744791 | 0.592 | 0.273 | 4.40E−108 |
| Tnfrsf1a | 2.83E−112 | 0.346845029 | 0.551 | 0.244 | 7.02E−108 |
| Gcnt2 | 3.56E−112 | 0.221618104 | 0.333 | 0.082 | 8.85E−108 |
| Ugt2b34 | 4.78E−112 | −0.290735843 | 0.918 | 0.874 | 1.19E−107 |
| Ell2 | 5.81E−112 | 0.371745977 | 0.72 | 0.416 | 1.44E−107 |
| Ly6e | 6.33E−112 | 0.42022985 | 0.68 | 0.386 | 1.57E−107 |
| Rps2 | 1.81E−110 | −0.44250848 | 0.675 | 0.757 | 4.49E−106 |
| Epb4.1 | 3.41E−110 | 0.349430673 | 0.617 | 0.304 | 8.48E−106 |
| Dscr3 | 6.96E−110 | 0.180232898 | 0.339 | 0.09 | 1.73E−105 |
| Ptpn2 | 1.69E−109 | 0.225408583 | 0.354 | 0.097 | 4.20E−105 |
| Zc3h13 | 9.21E−109 | 0.204542393 | 0.325 | 0.083 | 2.29E−104 |
| Clpx | 1.01E−108 | −0.306093278 | 0.818 | 0.783 | 2.51E−104 |
| Rps27l | 5.04E−108 | −0.361138678 | 0.855 | 0.849 | 1.25E−103 |
| Dst | 5.40E−108 | 0.329905553 | 0.507 | 0.206 | 1.34E−103 |
| Atl2 | 1.09E−107 | 0.321873809 | 0.674 | 0.361 | 2.72E−103 |
| Gm4076 | 1.71E−107 | −0.342157966 | 0.007 | 0.184 | 4.24E−103 |
| Lpgat1 | 3.04E−107 | 0.390146943 | 0.776 | 0.502 | 7.54E−103 |
| Gm20594 | 3.81E−107 | −0.559577825 | 0.108 | 0.294 | 9.47E−103 |
| Slc41a1 | 7.08E−107 | 0.110049944 | 0.168 | 0.011 | 1.76E−102 |
| Hmgcs1 | 8.42E−107 | −0.258009688 | 0.778 | 0.703 | 2.09E−102 |
| Esd | 2.48E−106 | −0.302906103 | 0.794 | 0.784 | 6.15E−102 |
| Oaz1 | 3.13E−106 | −0.339004868 | 0.759 | 0.75 | 7.77E−102 |
| Pptc7 | 8.30E−106 | 0.227095018 | 0.412 | 0.141 | 2.06E−101 |
| Sult1a1 | 1.33E−105 | 0.371023537 | 0.899 | 0.707 | 3.29E−101 |
| Ctif | 2.36E−105 | 0.18604392 | 0.312 | 0.078 | 5.85E−101 |
| Nfil3 | 2.49E−105 | 0.338730712 | 0.617 | 0.309 | 6.19E−101 |
| Gpx1 | 6.46E−105 | −0.222094695 | 1 | 0.999 | 1.60E−100 |
| Cyp3a11 | 6.66E−105 | −0.251219977 | 0.999 | 1 | 1.65E−100 |
| Aqp8 | 7.56E−105 | −0.331182913 | 0.444 | 0.505 | 1.88E−100 |
| Socs3 | 1.67E−104 | 0.194515821 | 0.456 | 0.183 | 4.14E−100 |
| Srrm4 | 2.29E−104 | 0.117065188 | 0.195 | 0.021 | 5.69E−100 |
| Apoa4 | 2.82E−104 | 0.609214916 | 0.604 | 0.339 | 7.01E−100 |
| Cml1 | 3.26E−104 | −0.277339974 | 0.803 | 0.757 | 8.09E−100 |
| Jak1 | 3.38E−104 | 0.362766687 | 0.648 | 0.342 | 8.40E−100 |
| Arid5b | 4.92E−104 | 0.232391653 | 0.472 | 0.186 | 1.22E−99 |
| Rasgef1b | 7.14E−104 | 0.174426927 | 0.279 | 0.06 | 1.77E−99 |
| Cers6 | 8.32E−104 | 0.1318056 | 0.198 | 0.022 | 2.07E−99 |
| Baiap2l1 | 9.30E−104 | 0.187103281 | 0.395 | 0.136 | 2.31E−99 |
| Rrs1 | 1.41E−103 | 0.167967739 | 0.314 | 0.082 | 3.51E−99 |
| Ralgapa2 | 2.43E−103 | 0.20791298 | 0.334 | 0.092 | 6.04E−99 |
| G6pc | 4.74E−103 | 0.341403933 | 0.948 | 0.752 | 1.18E−98 |
| Rlf | 7.11E−103 | 0.176204022 | 0.321 | 0.086 | 1.76E−98 |
| Ifitm3 | 2.36E−102 | 0.331673739 | 0.958 | 0.812 | 5.86E−98 |
| Zfp36 | 2.64E−102 | 0.407014847 | 0.776 | 0.522 | 6.55E−98 |
| Oaf | 7.72E−102 | −0.333766768 | 0.79 | 0.794 | 1.92E−97 |
| Mgst1 | 7.79E−102 | −0.322742924 | 1 | 0.997 | 1.93E−97 |
| Uqcrq | 1.17E−101 | −0.316451873 | 0.98 | 0.951 | 2.90E−97 |
| Garem | 4.69E−101 | 0.204881738 | 0.351 | 0.104 | 1.16E−96 |
| B2m | 6.16E−101 | 0.246889569 | 0.997 | 0.963 | 1.53E−96 |
| Man2a1 | 4.39E−100 | 0.369999358 | 0.83 | 0.583 | 1.09E−95 |
| Hebp1 | 4.64E−100 | −0.313009472 | 0.742 | 0.75 | 1.15E−95 |
| Sod1 | 5.22E−100 | −0.238388169 | 0.998 | 0.983 | 1.30E−95 |
| mt-Nd6 | 7.05E−100 | −0.515107457 | 0.389 | 0.577 | 1.75E−95 |
| Map3k5 | 9.85E−100 | 0.21643947 | 0.411 | 0.147 | 2.45E−95 |
| Hint2 | 1.16E−99 | −0.299309844 | 0.685 | 0.676 | 2.87E−95 |
| Ndufb6 | 1.92E−99 | −0.311202811 | 0.77 | 0.759 | 4.76E−95 |
| Palmd | 2.20E−99 | 0.284673594 | 0.51 | 0.219 | 5.47E−95 |
| Eef2 | 2.93E−99 | 0.289562847 | 0.979 | 0.873 | 7.28E−95 |
| 3110043O21Rik | 3.71E−99 | 0.171966272 | 0.282 | 0.065 | 9.20E−95 |
| Heatr1 | 4.46E−99 | 0.163145002 | 0.275 | 0.062 | 1.11E−94 |
| Mgll | 8.84E−99 | 0.321840174 | 0.739 | 0.449 | 2.19E−94 |
| Vtn | 1.05E−98 | 0.225160672 | 0.999 | 0.983 | 2.62E−94 |
| Aff4 | 3.03E−98 | 0.344186222 | 0.635 | 0.337 | 7.53E−94 |
| Pawr | 3.27E−98 | 0.116243505 | 0.217 | 0.035 | 8.13E−94 |
| Aldh1l1 | 6.75E−98 | 0.277372649 | 0.991 | 0.904 | 1.68E−93 |
| Tef | 7.02E−98 | 0.127953279 | 0.384 | 0.148 | 1.74E−93 |
| Wdr45b | 7.39E−98 | 0.246836045 | 0.459 | 0.181 | 1.83E−93 |
| Kdm4a | 1.53E−97 | 0.172058145 | 0.3 | 0.077 | 3.79E−93 |
| Gpd2 | 4.49E−97 | 0.23284812 | 0.393 | 0.135 | 1.12E−92 |
| Atp2a2 | 5.99E−97 | 0.350363266 | 0.771 | 0.495 | 1.49E−92 |
| Gadd45a | 2.39E−96 | 0.231268974 | 0.41 | 0.149 | 5.93E−92 |
| Mbnl2 | 3.18E−96 | 0.249293642 | 0.444 | 0.172 | 7.90E−92 |
| Eif1a | 3.51E−96 | 0.295219945 | 0.583 | 0.289 | 8.73E−92 |
| Igf1 | 5.12E−96 | 0.277138761 | 0.989 | 0.904 | 1.27E−91 |
| Acaa1b | 5.53E−96 | −0.263859024 | 0.968 | 0.969 | 1.37E−91 |
| Slc30a5 | 6.62E−96 | 0.207315427 | 0.378 | 0.126 | 1.65E−91 |
| Cyb5r3 | 1.40E−95 | 0.337684684 | 0.906 | 0.725 | 3.49E−91 |
| Il13ra1 | 1.49E−95 | 0.214635625 | 0.402 | 0.143 | 3.69E−91 |
| Dopey2 | 3.36E−95 | 0.19521799 | 0.33 | 0.097 | 8.35E−91 |
| Trak2 | 4.32E−95 | 0.13630483 | 0.264 | 0.062 | 1.07E−90 |
| Slc3a1 | 8.69E−95 | 0.290117272 | 0.464 | 0.189 | 2.16E−90 |
| Nop58 | 2.05E−94 | 0.257566653 | 0.428 | 0.162 | 5.10E−90 |
| Ttpal | 2.08E−94 | 0.129043255 | 0.243 | 0.051 | 5.15E−90 |
| Serpina1e | 2.45E−94 | 0.233787997 | 1 | 0.999 | 6.07E−90 |
| Gm2a | 3.18E−94 | 0.341145926 | 0.704 | 0.427 | 7.89E−90 |
| Ell | 4.24E−94 | 0.161665745 | 0.291 | 0.076 | 1.05E−89 |
| Por | 5.25E−94 | 0.290745567 | 0.898 | 0.675 | 1.30E−89 |
| Mvd | 5.90E−94 | −0.327327327 | 0.085 | 0.269 | 1.47E−89 |
| Sparcl1 | 7.09E−94 | −0.253616986 | 0 | 0.136 | 1.76E−89 |
| Rpl13a | 3.14E−93 | −0.314606263 | 0.858 | 0.868 | 7.80E−89 |
| Enpp2 | 5.14E−93 | 0.316882991 | 0.635 | 0.345 | 1.28E−88 |
| Kng1 | 6.56E−93 | 0.194558698 | 1 | 0.991 | 1.63E−88 |
| Mpc2 | 6.72E−93 | −0.268047152 | 0.887 | 0.857 | 1.67E−88 |
| Cep110 | 8.04E−93 | 0.192096419 | 0.362 | 0.121 | 2.00E−88 |
| Creb3l2 | 1.67E−92 | 0.161215013 | 0.278 | 0.068 | 4.14E−88 |
| Ambp | 2.13E−92 | 0.211238054 | 0.999 | 0.993 | 5.28E−88 |
| Dstn | 2.99E−92 | 0.271381717 | 0.664 | 0.37 | 7.44E−88 |
| Cyp1a2 | 3.17E−92 | −0.481919967 | 0.685 | 0.794 | 7.86E−88 |
| Pfkfb2 | 1.80E−91 | 0.160198891 | 0.27 | 0.067 | 4.47E−87 |
| Ahsg | 1.83E−91 | 0.171853168 | 1 | 1 | 4.55E−87 |
| Sec14l4 | 1.91E−91 | 0.285169227 | 0.724 | 0.436 | 4.74E−87 |
| Slc38a3 | 4.51E−91 | 0.292400046 | 0.971 | 0.858 | 1.12E−86 |
| Xdh | 1.64E−90 | 0.27224472 | 0.541 | 0.258 | 4.08E−86 |
| Cfhr2 | 2.62E−90 | −0.246923339 | 0.949 | 0.868 | 6.51E−86 |
| Slc16a6 | 3.77E−90 | 0.21530975 | 0.251 | 0.054 | 9.35E−86 |
| Spin1 | 4.11E−90 | 0.232119983 | 0.478 | 0.206 | 1.02E−85 |
| Coq10b | 6.51E−90 | 0.153862213 | 0.313 | 0.093 | 1.62E−85 |
| Dusp1 | 1.08E−89 | 0.300628481 | 0.539 | 0.259 | 2.67E−85 |
| Ubc | 2.12E−89 | −0.273242853 | 0.907 | 0.872 | 5.27E−85 |
| Aadac | 3.37E−89 | −0.230855362 | 0.952 | 0.86 | 8.37E−85 |
| Txnrd1 | 3.83E−89 | 0.314979852 | 0.721 | 0.441 | 9.50E−85 |
| Serinc3 | 1.76E−88 | 0.293964974 | 0.713 | 0.433 | 4.36E−84 |
| Slc20a1 | 2.48E−88 | 0.125755272 | 0.279 | 0.076 | 6.17E−84 |
| Dbi | 2.79E−88 | 0.243173797 | 0.999 | 0.971 | 6.93E−84 |
| C8a | 5.37E−88 | 0.294064237 | 0.978 | 0.887 | 1.33E−83 |
| Eci2 | 1.87E−87 | 0.288159513 | 0.896 | 0.698 | 4.64E−83 |
| Chd1 | 1.92E−87 | 0.213257509 | 0.42 | 0.166 | 4.77E−83 |
| Elovl2 | 2.69E−87 | 0.285038606 | 0.912 | 0.699 | 6.69E−83 |
| Atf6 | 2.82E−87 | 0.280521162 | 0.553 | 0.273 | 6.99E−83 |
| Lrp6 | 3.24E−87 | 0.270740438 | 0.505 | 0.232 | 8.05E−83 |
| Maff | 9.72E−87 | 0.118590539 | 0.222 | 0.048 | 2.41E−82 |
| Nr4a1 | 1.19E−86 | 0.13631399 | 0.207 | 0.036 | 2.96E−82 |
| Myo18a | 4.19E−86 | 0.233717966 | 0.476 | 0.211 | 1.04E−81 |
| Sigmar1 | 6.27E−86 | −0.282364617 | 0.482 | 0.514 | 1.56E−81 |
| Gck | 6.72E−86 | 0.260705425 | 0.557 | 0.278 | 1.67E−81 |
| D5Ertd579e | 7.81E−86 | 0.197825601 | 0.328 | 0.104 | 1.94E−81 |
| Creg1 | 9.18E−86 | −0.235698597 | 0.966 | 0.926 | 2.28E−81 |
| Tnfrsf12a | 1.24E−85 | 0.114819395 | 0.233 | 0.052 | 3.07E−81 |
| D630036G22Rik | 1.31E−85 | 0.147427696 | 0.268 | 0.071 | 3.25E−81 |
| Atp5f1 | 1.90E−85 | −0.217555876 | 0.943 | 0.869 | 4.71E−81 |
| Zfp445 | 2.06E−85 | 0.159662672 | 0.371 | 0.138 | 5.12E−81 |
| Id3 | 2.29E−85 | 0.399520373 | 0.688 | 0.426 | 5.68E−81 |
| Ndufa6 | 2.67E−85 | −0.25272382 | 0.846 | 0.798 | 6.62E−81 |
| Nfkbiz | 3.10E−85 | 0.157772695 | 0.249 | 0.059 | 7.69E−81 |
| Kansl1 | 3.35E−85 | 0.146121195 | 0.286 | 0.08 | 8.31E−81 |
| Asl | 3.71E−85 | 0.297673772 | 0.934 | 0.787 | 9.22E−81 |
| Tgm2 | 8.13E−85 | 0.316575272 | 0.524 | 0.258 | 2.02E−80 |
| Cpb2 | 9.20E−85 | 0.280302458 | 0.959 | 0.813 | 2.28E−80 |
| Uroc1 | 2.32E−84 | 0.329587334 | 0.837 | 0.615 | 5.77E−80 |
| Aox3 | 2.71E−84 | 0.275388909 | 0.989 | 0.894 | 6.73E−80 |
| Ddi2 | 3.48E−84 | 0.302519699 | 0.642 | 0.365 | 8.63E−80 |
| Etv6 | 6.75E−84 | 0.143861801 | 0.266 | 0.069 | 1.68E−79 |
| Aim1 | 6.84E−84 | 0.132131086 | 0.302 | 0.098 | 1.70E−79 |
| Tsr1 | 7.09E−84 | 0.149561769 | 0.311 | 0.097 | 1.76E−79 |
| Flnb | 1.86E−83 | 0.212710481 | 0.376 | 0.139 | 4.61E−79 |
| Rab30 | 2.08E−83 | 0.112636253 | 0.235 | 0.056 | 5.16E−79 |
| Fgf1 | 2.27E−83 | 0.287983029 | 0.647 | 0.369 | 5.65E−79 |
| Cobll1 | 3.65E−83 | 0.158603105 | 0.353 | 0.13 | 9.07E−79 |
| Arl4d | 4.26E−83 | −0.328520283 | 0.326 | 0.437 | 1.06E−78 |
| H6pd | 1.81E−82 | 0.300492669 | 0.825 | 0.595 | 4.50E−78 |
| Tsc22d2 | 2.10E−82 | 0.165988347 | 0.349 | 0.124 | 5.21E−78 |
| Ddx5 | 6.27E−82 | 0.251675045 | 0.778 | 0.515 | 1.56E−77 |
| Megf9 | 6.42E−82 | 0.211011671 | 0.401 | 0.158 | 1.59E−77 |
| Ctsb | 6.42E−82 | 0.268200419 | 0.943 | 0.781 | 1.60E−77 |
| Il6st | 7.72E−82 | 0.262348153 | 0.544 | 0.273 | 1.92E−77 |
| Mttp | 8.01E−82 | 0.296651415 | 0.888 | 0.697 | 1.99E−77 |
| Orm2 | 9.10E−82 | 0.144101366 | 0.286 | 0.09 | 2.26E−77 |
| Gde1 | 2.65E−81 | 0.222485647 | 0.531 | 0.263 | 6.58E−77 |
| Gm9843 | 2.74E−81 | −0.343302667 | 0.197 | 0.299 | 6.81E−77 |
| Zfp36l2 | 3.05E−81 | 0.103487303 | 0.215 | 0.05 | 7.57E−77 |
| Slc35g1 | 3.63E−81 | 0.166162728 | 0.36 | 0.133 | 9.02E−77 |
| Trp53inp1 | 4.94E−81 | 0.269278401 | 0.582 | 0.308 | 1.23E−76 |
| Rmnd5a | 6.65E−81 | 0.273473023 | 0.628 | 0.352 | 1.65E−76 |
| Fads1 | 7.20E−81 | 0.267264464 | 0.889 | 0.663 | 1.79E−76 |
| Nrp1 | 1.23E−80 | 0.268796793 | 0.518 | 0.253 | 3.05E−76 |
| Rbm47 | 1.73E−80 | 0.21054927 | 0.475 | 0.218 | 4.29E−76 |
| Lrp1 | 1.82E−80 | 0.310226628 | 0.831 | 0.591 | 4.51E−76 |
| Gm10250 | 1.89E−80 | −0.30688429 | 0.124 | 0.251 | 4.69E−76 |
| C4bp | 2.56E−80 | 0.26668764 | 0.948 | 0.788 | 6.36E−76 |
| Prox1 | 2.59E−80 | 0.31520499 | 0.622 | 0.351 | 6.44E−76 |
| Cebpa | 3.88E−80 | −0.260692112 | 0.688 | 0.672 | 9.63E−76 |
| Angptl4 | 3.88E−80 | 0.315315425 | 0.838 | 0.629 | 9.63E−76 |
| Clip1 | 4.78E−80 | 0.166877376 | 0.438 | 0.196 | 1.19E−75 |
| Cish | 6.18E−80 | 0.172549219 | 0.302 | 0.093 | 1.53E−75 |
| Lbp | 1.00E−79 | 0.264248666 | 0.605 | 0.333 | 2.49E−75 |
| Hs6st1 | 1.20E−79 | 0.206647988 | 0.524 | 0.261 | 2.99E−75 |
| Zfp683 | 1.36E−79 | −0.421689112 | 0 | 0.116 | 3.38E−75 |
| Clu | 1.66E−79 | 0.2220984 | 0.995 | 0.958 | 4.13E−75 |
| Picalm | 1.74E−79 | 0.276902165 | 0.648 | 0.376 | 4.32E−75 |
| Acly | 1.84E−79 | 0.287663867 | 0.715 | 0.445 | 4.57E−75 |
| Cox7b | 1.88E−79 | −0.304638563 | 0.91 | 0.878 | 4.67E−75 |
| Dcxr | 1.93E−79 | −0.271339616 | 0.629 | 0.631 | 4.79E−75 |
| Myd88 | 2.01E−79 | 0.121633496 | 0.254 | 0.068 | 5.00E−75 |
| Malat1 | 3.00E−79 | −0.652623643 | 0.704 | 0.78 | 7.46E−75 |
| Elovl3 | 5.41E−79 | −0.25229278 | 0.838 | 0.827 | 1.34E−74 |
| Hs3st3b1 | 1.01E−78 | 0.242350838 | 0.501 | 0.241 | 2.50E−74 |
| Atp5h | 1.29E−78 | −0.351854327 | 0.688 | 0.682 | 3.20E−74 |
| Ipo7 | 2.63E−78 | 0.176153152 | 0.402 | 0.168 | 6.52E−74 |
| Nit2 | 2.92E−78 | −0.196867848 | 0.62 | 0.577 | 7.24E−74 |
| Casp8 | 2.93E−78 | 0.130902157 | 0.325 | 0.118 | 7.27E−74 |
| Pdp2 | 3.49E−78 | 0.130918419 | 0.298 | 0.102 | 8.67E−74 |
| Uox | 4.04E−78 | 0.189986074 | 0.998 | 0.965 | 1.00E−73 |
| Azin1 | 4.14E−78 | 0.268891169 | 0.59 | 0.322 | 1.03E−73 |
| Tmed5 | 5.52E−78 | 0.296018777 | 0.74 | 0.492 | 1.37E−73 |
| Ppp2r5a | 6.09E−78 | −0.263513608 | 0.593 | 0.594 | 1.51E−73 |
| Lrrc16a | 7.34E−78 | 0.127437209 | 0.244 | 0.063 | 1.82E−73 |
| Tbl1xr1 | 8.85E−78 | 0.204315292 | 0.418 | 0.176 | 2.20E−73 |
| Copa | 1.43E−77 | 0.251410027 | 0.633 | 0.363 | 3.55E−73 |
| Gm20721 | 1.75E−77 | 0.117268341 | 0.229 | 0.056 | 4.35E−73 |
| Hist1h1c | 2.20E−77 | −0.331045753 | 0.24 | 0.372 | 5.47E−73 |
| Gnl3 | 4.69E−77 | 0.242175469 | 0.454 | 0.204 | 1.16E−72 |
| Bhmt | 6.30E−77 | −0.236483408 | 0.988 | 0.956 | 1.56E−72 |
| Nr1d2 | 1.05E−76 | 0.159943118 | 0.411 | 0.179 | 2.61E−72 |
| Sord | 1.07E−76 | −0.228716308 | 0.964 | 0.931 | 2.65E−72 |
| mt-Tm | 1.17E−76 | 0.126497553 | 0.283 | 0.093 | 2.91E−72 |
| Rhobtb1 | 1.59E−76 | 0.178172942 | 0.406 | 0.172 | 3.94E−72 |
| Ddhd2 | 1.79E−76 | 0.105524805 | 0.267 | 0.083 | 4.45E−72 |
| Pik3c2a | 2.23E−76 | 0.232920912 | 0.444 | 0.198 | 5.53E−72 |
| Sec14l2 | 4.97E−76 | −0.220330298 | 0.905 | 0.856 | 1.23E−71 |
| Ythdc1 | 5.91E−76 | 0.15157378 | 0.369 | 0.15 | 1.47E−71 |
| Smoc1 | 7.84E−76 | 0.212439914 | 0.49 | 0.238 | 1.95E−71 |
| Plec | 8.34E−76 | 0.197499812 | 0.424 | 0.186 | 2.07E−71 |
| Pros1 | 8.47E−76 | 0.121465912 | 0.373 | 0.158 | 2.10E−71 |
| Hipk3 | 1.03E−75 | 0.225614187 | 0.472 | 0.223 | 2.56E−71 |
| Bag3 | 1.60E−75 | 0.187468684 | 0.376 | 0.148 | 3.97E−71 |
| Ctsl | 1.66E−75 | 0.256520866 | 0.976 | 0.857 | 4.11E−71 |
| Ppib | 1.75E−75 | −0.223880669 | 0.823 | 0.767 | 4.33E−71 |
| Suco | 2.17E−75 | 0.112486448 | 0.307 | 0.113 | 5.39E−71 |
| Angptl3 | 2.21E−75 | −0.18094369 | 0.993 | 0.944 | 5.49E−71 |
| Iqgap2 | 2.81E−75 | 0.283192936 | 0.879 | 0.677 | 6.97E−71 |
| Hhex | 3.00E−75 | −0.283483187 | 0.229 | 0.307 | 7.46E−71 |
| Acsl4 | 3.34E−75 | 0.250793464 | 0.588 | 0.323 | 8.30E−71 |
| Nr1i2 | 4.43E−75 | 0.198788095 | 0.505 | 0.252 | 1.10E−70 |
| Clp1 | 5.40E−75 | 0.153048593 | 0.307 | 0.103 | 1.34E−70 |
| Ddc | 9.70E−75 | −0.216024433 | 0.475 | 0.46 | 2.41E−70 |
| Lrpprc | 1.15E−74 | 0.257016288 | 0.662 | 0.398 | 2.85E−70 |
| Slc38a4 | 1.26E−74 | 0.244607846 | 0.976 | 0.877 | 3.13E−70 |
| Sc4mol | 1.49E−74 | −0.23053256 | 0.669 | 0.642 | 3.71E−70 |
| Lurap1l | 1.62E−74 | 0.241965054 | 0.563 | 0.301 | 4.03E−70 |
| Sik1 | 1.73E−74 | 0.111557849 | 0.257 | 0.078 | 4.31E−70 |
| Akap11 | 2.07E−74 | 0.187807202 | 0.392 | 0.164 | 5.15E−70 |
| Taf2 | 2.10E−74 | 0.146241085 | 0.25 | 0.067 | 5.21E−70 |
| Cyp2c70 | 3.28E−74 | 0.25600888 | 0.971 | 0.834 | 8.15E−70 |
| Lipg | 3.43E−74 | 0.107109358 | 0.215 | 0.051 | 8.52E−70 |
| Tirap | 3.44E−74 | 0.131022584 | 0.304 | 0.106 | 8.54E−70 |
| Gstp1 | 5.57E−74 | 0.197542109 | 0.962 | 0.822 | 1.38E−69 |
| Spty2d1 | 6.27E−74 | 0.105732944 | 0.27 | 0.087 | 1.56E−69 |
| Chd7 | 6.41E−74 | 0.135402444 | 0.27 | 0.085 | 1.59E−69 |
| Sc5d | 6.91E−74 | −0.107743918 | 0.907 | 0.776 | 1.72E−69 |
| Hsd3b3 | 7.80E−74 | −0.226222481 | 0.631 | 0.605 | 1.94E−69 |
| Sec24a | 9.16E−74 | 0.188750112 | 0.504 | 0.255 | 2.28E−69 |
| Csmp1 | 1.23E−73 | 0.127548152 | 0.319 | 0.122 | 3.05E−69 |
| Sepp1 | 1.42E−73 | 0.16148927 | 1 | 1 | 3.51E−69 |
| Gstm3 | 1.76E−73 | −0.470077922 | 0.343 | 0.414 | 4.37E−69 |
| Ass1 | 2.23E−73 | 0.216965784 | 0.996 | 0.962 | 5.55E−69 |
| Cfi | 2.41E−73 | 0.226479679 | 0.988 | 0.905 | 5.99E−69 |
| Fbxo34 | 3.57E−73 | 0.143040285 | 0.301 | 0.104 | 8.86E−69 |
| Ppp1r3b | 5.81E−73 | 0.259418401 | 0.642 | 0.38 | 1.44E−68 |
| Arhgef12 | 7.54E−73 | 0.280147378 | 0.667 | 0.408 | 1.87E−68 |
| Usp9x | 1.10E−72 | 0.233121474 | 0.556 | 0.299 | 2.73E−68 |
| Rapgef2 | 1.13E−72 | 0.117395681 | 0.237 | 0.067 | 2.82E−68 |
| Arnt | 1.36E−72 | 0.102920838 | 0.212 | 0.052 | 3.38E−68 |
| Fbxo31 | 1.45E−72 | 0.121941263 | 0.505 | 0.273 | 3.61E−68 |
| Ranbp2 | 1.48E−72 | 0.208744477 | 0.596 | 0.338 | 3.67E−68 |
| Mak16 | 1.48E−72 | 0.102725211 | 0.269 | 0.087 | 3.67E−68 |
| Fbp1 | 1.92E−72 | 0.236214341 | 0.997 | 0.938 | 4.78E−68 |
| Trip11 | 2.55E−72 | 0.163424021 | 0.429 | 0.2 | 6.32E−68 |
| Ndufc2 | 2.97E−72 | −0.202304309 | 0.639 | 0.579 | 7.36E−68 |
| 2900026A02Rik | 4.76E−72 | 0.175166554 | 0.416 | 0.187 | 1.18E−67 |
| Nr2f6 | 4.80E−72 | −0.219631094 | 0.417 | 0.434 | 1.19E−67 |
| Rc3h1 | 4.90E−72 | 0.165519544 | 0.33 | 0.122 | 1.22E−67 |
| mt-Tq | 5.18E−72 | 0.113221186 | 0.218 | 0.057 | 1.29E−67 |
| Ash1l | 6.20E−72 | 0.131678753 | 0.39 | 0.175 | 1.54E−67 |
| Ywhag | 6.59E−72 | 0.215838932 | 0.563 | 0.308 | 1.64E−67 |
| Dpy19l1 | 7.13E−72 | 0.23352829 | 0.531 | 0.277 | 1.77E−67 |
| Srpr | 7.66E−72 | 0.223990237 | 0.647 | 0.387 | 1.90E−67 |
| Cmah | 7.80E−72 | 0.247781083 | 0.721 | 0.465 | 1.94E−67 |
| Ssfa2 | 8.53E−72 | 0.162814615 | 0.354 | 0.141 | 2.12E−67 |
| Apoc3 | 1.50E−71 | 0.187850611 | 0.999 | 0.993 | 3.73E−67 |
| Atp5j | 2.76E−71 | −0.234964444 | 0.782 | 0.757 | 6.86E−67 |
| Atp1a1 | 3.01E−71 | 0.261718912 | 0.709 | 0.457 | 7.48E−67 |
| Abhd2 | 4.33E−71 | 0.14425027 | 0.302 | 0.105 | 1.07E−66 |
| Ppp1r15a | 5.70E−71 | 0.157408911 | 0.336 | 0.13 | 1.42E−66 |
| Calr | 6.41E−71 | 0.232562138 | 0.972 | 0.885 | 1.59E−66 |
| Cyp2c55 | 9.78E−71 | −0.316565473 | 0.103 | 0.243 | 2.43E−66 |
| Skil | 1.08E−70 | 0.126702219 | 0.269 | 0.086 | 2.67E−66 |
| Fdx1 | 1.35E−70 | −0.213846254 | 0.669 | 0.625 | 3.35E−66 |
| Gls2 | 2.03E−70 | 0.296143664 | 0.868 | 0.657 | 5.05E−66 |
| Alas1 | 2.26E−70 | 0.283195713 | 0.905 | 0.729 | 5.61E−66 |
| Nup50 | 2.39E−70 | 0.121192082 | 0.374 | 0.164 | 5.94E−66 |
| Cdip1 | 3.30E−70 | 0.161158186 | 0.514 | 0.273 | 8.19E−66 |
| Pklr | 3.64E−70 | 0.25984035 | 0.624 | 0.368 | 9.04E−66 |
| Arf6 | 3.89E−70 | 0.132424754 | 0.407 | 0.189 | 9.65E−66 |
| Acaca | 4.34E−70 | 0.139911693 | 0.301 | 0.106 | 1.08E−65 |
| Ftl1 | 4.45E−70 | −0.346327339 | 0.394 | 0.409 | 1.10E−65 |
| Tmem205 | 5.13E−70 | −0.196745573 | 0.978 | 0.934 | 1.27E−65 |
| Pnpla7 | 6.70E−70 | 0.251226201 | 0.807 | 0.572 | 1.66E−65 |
| Hsd17b13 | 6.70E−70 | 0.351593878 | 0.907 | 0.74 | 1.66E−65 |
| Ech1 | 1.07E−69 | −0.191877671 | 0.908 | 0.856 | 2.66E−65 |
| Osbpl1a | 1.17E−69 | 0.247696779 | 0.721 | 0.473 | 2.89E−65 |
| Baz1a | 1.29E−69 | 0.111993205 | 0.249 | 0.077 | 3.21E−65 |
| Arl8b | 1.48E−69 | 0.198760529 | 0.468 | 0.228 | 3.68E−65 |
| Tprkb | 1.70E−69 | −0.254084951 | 0.508 | 0.528 | 4.23E−65 |
| Comt | 2.11E−69 | 0.220396792 | 0.99 | 0.931 | 5.25E−65 |
| Nucks1 | 2.41E−69 | 0.249253158 | 0.683 | 0.43 | 5.99E−65 |
| Pim3 | 2.79E−69 | 0.280137371 | 0.764 | 0.537 | 6.92E−65 |
| Nek7 | 4.21E−69 | 0.204786835 | 0.517 | 0.271 | 1.04E−64 |
| Tm9sf2 | 5.17E−69 | 0.208759009 | 0.616 | 0.362 | 1.28E−64 |
| P4hb | 5.86E−69 | 0.204526132 | 0.995 | 0.941 | 1.46E−64 |
| Ldha | 7.13E−69 | 0.234882634 | 0.974 | 0.896 | 1.77E−64 |
| Nolc1 | 7.21E−69 | 0.14652588 | 0.338 | 0.134 | 1.79E−64 |
| Ripk1 | 8.04E−69 | 0.174862366 | 0.407 | 0.183 | 2.00E−64 |
| Akr1c20 | 9.26E−69 | −0.230255116 | 0.563 | 0.564 | 2.30E−64 |
| Cldn1 | 9.68E−69 | −0.246774022 | 0.21 | 0.306 | 2.40E−64 |
| Mtus1 | 1.13E−68 | 0.268076242 | 0.674 | 0.42 | 2.80E−64 |
| Ibtk | 1.14E−68 | 0.188149661 | 0.485 | 0.247 | 2.82E−64 |
| Kdm5c | 1.27E−68 | 0.147833422 | 0.338 | 0.134 | 3.15E−64 |
| Rdh7 | 1.46E−68 | −0.197994968 | 0.993 | 0.969 | 3.62E−64 |
| Me1 | 1.64E−68 | 0.229105587 | 0.699 | 0.447 | 4.06E−64 |
| Ptplad1 | 1.76E−68 | 0.255273084 | 0.741 | 0.496 | 4.37E−64 |
| Gm20528 | 1.77E−68 | 0.118200587 | 0.246 | 0.076 | 4.39E−64 |
| Scarb2 | 6.53E−68 | 0.19345963 | 0.603 | 0.353 | 1.62E−63 |
| Cnbp | 8.69E−68 | −0.203726646 | 0.828 | 0.778 | 2.16E−63 |
| Hagh | 1.07E−67 | −0.157822564 | 0.933 | 0.858 | 2.65E−63 |
| Prdx4 | 1.30E−67 | −0.205807709 | 0.653 | 0.607 | 3.24E−63 |
| Cep85 | 1.35E−67 | 0.171686021 | 0.383 | 0.167 | 3.35E−63 |
| Cyp4a12a | 1.81E−67 | 0.259776426 | 0.823 | 0.597 | 4.49E−63 |
| Fas | 1.94E−67 | 0.145849731 | 0.309 | 0.114 | 4.81E−63 |
| Hdlbp | 3.10E−67 | 0.267317937 | 0.894 | 0.713 | 7.69E−63 |
| St3gal1 | 4.28E−67 | 0.192410856 | 0.454 | 0.221 | 1.06E−62 |
| Gpc4 | 4.76E−67 | 0.16318479 | 0.349 | 0.141 | 1.18E−62 |
| 2310044G17Rik | 5.00E−67 | 0.144057038 | 0.312 | 0.117 | 1.24E−62 |
| Efhd2 | 8.01E−67 | 0.105846616 | 0.34 | 0.148 | 1.99E−62 |
| Mctp2 | 1.20E−66 | 0.108025959 | 0.216 | 0.057 | 2.97E−62 |
| Dhrs3 | 1.24E−66 | −0.21828148 | 0.451 | 0.465 | 3.09E−62 |
| Nedd4 | 1.43E−66 | 0.259978699 | 0.762 | 0.524 | 3.56E−62 |
| Mafb | 2.56E−66 | 0.216162055 | 0.577 | 0.33 | 6.37E−62 |
| Aplp2 | 3.49E−66 | 0.234842456 | 0.844 | 0.625 | 8.67E−62 |
| Rpl14 | 4.02E−66 | −0.261157069 | 0.559 | 0.574 | 9.98E−62 |
| Spred1 | 4.97E−66 | 0.110303756 | 0.234 | 0.069 | 1.23E−61 |
| Cap1 | 6.06E−66 | 0.156999287 | 0.42 | 0.202 | 1.50E−61 |
| Nploc4 | 6.75E−66 | 0.164369249 | 0.436 | 0.214 | 1.68E−61 |
| Atl3 | 7.24E−66 | 0.191666389 | 0.506 | 0.27 | 1.80E−61 |
| Wdr43 | 9.77E−66 | 0.116370658 | 0.343 | 0.146 | 2.43E−61 |
| Kif21a | 1.14E−65 | 0.240302185 | 0.537 | 0.293 | 2.82E−61 |
| Macf1 | 1.72E−65 | 0.203727526 | 0.447 | 0.219 | 4.27E−61 |
| Cmbl | 1.79E−65 | −0.198257983 | 0.724 | 0.678 | 4.46E−61 |
| Alas2 | 2.13E−65 | 0.17568703 | 0.586 | 0.344 | 5.29E−61 |
| Actr2 | 2.17E−65 | 0.170960453 | 0.638 | 0.394 | 5.39E−61 |
| Gsk3b | 2.37E−65 | 0.159364703 | 0.41 | 0.193 | 5.90E−61 |
| Myo1b | 2.40E−65 | 0.247482254 | 0.776 | 0.542 | 5.96E−61 |
| Pcm1 | 2.44E−65 | 0.153416983 | 0.343 | 0.142 | 6.06E−61 |
| Tesk2 | 2.49E−65 | 0.118095034 | 0.229 | 0.066 | 6.17E−61 |
| Ddx21 | 2.61E−65 | 0.186649034 | 0.448 | 0.22 | 6.49E−61 |
| Ahr | 2.81E−65 | 0.139590211 | 0.349 | 0.151 | 6.97E−61 |
| Itpr1 | 3.09E−65 | 0.161076343 | 0.356 | 0.152 | 7.68E−61 |
| Uba1 | 3.33E−65 | 0.23027391 | 0.662 | 0.416 | 8.27E−61 |
| Rap1b | 3.43E−65 | 0.174034663 | 0.491 | 0.258 | 8.53E−61 |
| Apoa2 | 3.55E−65 | −0.174241915 | 1 | 1 | 8.81E−61 |
| Lrrc28 | 3.89E−65 | 0.107467233 | 0.27 | 0.094 | 9.66E−61 |
| Gdap2 | 5.12E−65 | 0.118267773 | 0.287 | 0.104 | 1.27E−60 |
| Srsf4 | 5.16E−65 | 0.109628861 | 0.318 | 0.132 | 1.28E−60 |
| Nop14 | 9.00E−65 | 0.120006548 | 0.288 | 0.108 | 2.24E−60 |
| Copz1 | 9.86E−65 | 0.221023165 | 0.631 | 0.385 | 2.45E−60 |
| Kmo | 9.94E−65 | 0.262269388 | 0.891 | 0.732 | 2.47E−60 |
| Slc2a2 | 1.09E−64 | 0.218249447 | 0.722 | 0.479 | 2.70E−60 |
| Plbd2 | 1.24E−64 | 0.169456289 | 0.42 | 0.199 | 3.08E−60 |
| Ahsa1 | 1.36E−64 | 0.126578677 | 0.458 | 0.24 | 3.38E−60 |
| Ythdf3 | 1.76E−64 | 0.174850983 | 0.474 | 0.243 | 4.37E−60 |
| Efna1 | 2.11E−64 | 0.256152989 | 0.551 | 0.309 | 5.25E−60 |
| Lpin2 | 2.50E−64 | 0.232988713 | 0.817 | 0.597 | 6.20E−60 |
| Ak3 | 2.62E−64 | 0.224559231 | 0.893 | 0.7 | 6.50E−60 |
| Dnttip2 | 2.63E−64 | 0.166547988 | 0.418 | 0.199 | 6.54E−60 |
| Supt6 | 2.69E−64 | 0.166662973 | 0.338 | 0.137 | 6.69E−60 |
| Wasl | 3.59E−64 | 0.178274463 | 0.449 | 0.223 | 8.92E−60 |
| Agpat6 | 4.04E−64 | 0.242639639 | 0.749 | 0.517 | 1.00E−59 |
| Gbe1 | 4.07E−64 | 0.183485273 | 0.577 | 0.338 | 1.01E−59 |
| Lrrc8a | 4.35E−64 | 0.100902509 | 0.255 | 0.085 | 1.08E−59 |
| Il15ra | 4.78E−64 | 0.129225351 | 0.314 | 0.125 | 1.19E−59 |
| Cyp39a1 | 5.01E−64 | 0.136555896 | 0.295 | 0.11 | 1.24E−59 |
| Cd1d1 | 5.24E−64 | −0.182746213 | 0.644 | 0.612 | 1.30E−59 |
| Nme1 | 5.76E−64 | −0.195199734 | 0.674 | 0.622 | 1.43E−59 |
| Snd1 | 6.86E−64 | 0.18406298 | 0.549 | 0.312 | 1.70E−59 |
| Rbp1 | 7.01E−64 | 0.118452694 | 0.234 | 0.069 | 1.74E−59 |
| Rcl1 | 1.12E−63 | 0.273905375 | 0.734 | 0.514 | 2.78E−59 |
| Mknk2 | 1.34E−63 | 0.1323349 | 0.412 | 0.205 | 3.33E−59 |
| Ndufb4 | 2.08E−63 | −0.233018121 | 0.486 | 0.49 | 5.18E−59 |
| Phf20l1 | 2.74E−63 | 0.123806334 | 0.422 | 0.212 | 6.81E−59 |
| Map3k1 | 2.99E−63 | 0.100043219 | 0.184 | 0.044 | 7.43E−59 |
| Gpam | 3.62E−63 | 0.169852715 | 0.417 | 0.199 | 9.00E−59 |
| Nipa2 | 4.23E−63 | 0.129532764 | 0.363 | 0.162 | 1.05E−58 |
| Bsdc1 | 4.31E−63 | 0.137035108 | 0.381 | 0.178 | 1.07E−58 |
| Riok2 | 4.51E−63 | 0.118813963 | 0.302 | 0.118 | 1.12E−58 |
| Cebpz | 4.87E−63 | 0.101120661 | 0.447 | 0.236 | 1.21E−58 |
| Zfp36l1 | 8.15E−63 | 0.271823824 | 0.636 | 0.402 | 2.02E−58 |
| Hif1a | 1.10E−62 | 0.13634532 | 0.366 | 0.166 | 2.72E−58 |
| Tnfrsf1b | 1.17E−62 | 0.112815211 | 0.257 | 0.087 | 2.90E−58 |
| Irf9 | 1.69E−62 | 0.102766206 | 0.329 | 0.145 | 4.18E−58 |
| Ccnt1 | 1.79E−62 | 0.133253027 | 0.307 | 0.119 | 4.44E−58 |
| Lmbrd2 | 1.97E−62 | 0.121703677 | 0.415 | 0.209 | 4.88E−58 |
| Cryz | 2.40E−62 | 0.137185877 | 0.468 | 0.251 | 5.96E−58 |
| Fgd6 | 2.54E−62 | 0.16919996 | 0.348 | 0.147 | 6.30E−58 |
| Larp4 | 2.70E−62 | 0.156912192 | 0.396 | 0.184 | 6.70E−58 |
| Atp13a3 | 4.02E−62 | 0.197384706 | 0.65 | 0.41 | 9.98E−58 |
| Fndc3b | 5.18E−62 | 0.151940318 | 0.276 | 0.096 | 1.29E−57 |
| Pum2 | 5.90E−62 | 0.14053782 | 0.382 | 0.177 | 1.47E−57 |
| D4Wsu53e | 6.52E−62 | −0.201263472 | 0.393 | 0.385 | 1.62E−57 |
| Slc27a2 | 1.00E−61 | 0.192264004 | 0.991 | 0.949 | 2.49E−57 |
| Wdr26 | 1.03E−61 | 0.207089713 | 0.646 | 0.406 | 2.56E−57 |
| Gns | 1.32E−61 | 0.166751532 | 0.516 | 0.288 | 3.28E−57 |
| Nrn1 | 1.50E−61 | −0.19828092 | 0.704 | 0.675 | 3.72E−57 |
| Pex11a | 1.58E−61 | 0.185588983 | 0.681 | 0.441 | 3.93E−57 |
| Enpep | 2.39E−61 | 0.192022994 | 0.374 | 0.166 | 5.93E−57 |
| Nr1i3 | 2.91E−61 | −0.317940692 | 0.294 | 0.377 | 7.23E−57 |
| Mup1 | 3.07E−61 | −0.490972296 | 0.609 | 0.583 | 7.63E−57 |
| Lrrc58 | 3.25E−61 | 0.196709613 | 0.597 | 0.359 | 8.07E−57 |
| Dync1h1 | 3.32E−61 | 0.187521298 | 0.428 | 0.21 | 8.24E−57 |
| Srrm2 | 3.41E−61 | 0.136584863 | 0.536 | 0.313 | 8.47E−57 |
| Coa6 | 4.11E−61 | −0.242879305 | 0.344 | 0.373 | 1.02E−56 |
| Slc16a10 | 4.41E−61 | 0.194608601 | 0.547 | 0.315 | 1.09E−56 |
| Crcp | 4.75E−61 | 0.135362988 | 0.41 | 0.203 | 1.18E−56 |
| Gclc | 4.81E−61 | −0.13277321 | 0.808 | 0.718 | 1.20E−56 |
| Vkorc1 | 6.39E−61 | −0.206999378 | 0.545 | 0.54 | 1.59E−56 |
| Sfpq | 7.82E−61 | 0.159417591 | 0.425 | 0.211 | 1.94E−56 |
| Selk | 8.43E−61 | −0.155942939 | 0.545 | 0.486 | 2.09E−56 |
| Dusp6 | 9.28E−61 | 0.205655249 | 0.491 | 0.261 | 2.30E−56 |
| Minos1 | 1.00E−60 | −0.201913176 | 0.638 | 0.595 | 2.49E−56 |
| Mir692-1 | 1.02E−60 | −0.20556011 | 0.012 | 0.126 | 2.54E−56 |
| Ccrn4l | 1.37E−60 | 0.108176603 | 0.243 | 0.08 | 3.41E−56 |
| Slco1b2 | 1.44E−60 | −0.207101647 | 0.976 | 0.951 | 3.57E−56 |
| Scamp1 | 1.64E−60 | 0.122878314 | 0.398 | 0.197 | 4.08E−56 |
| Scyl2 | 1.65E−60 | 0.130702939 | 0.344 | 0.151 | 4.09E−56 |
| Farp1 | 1.94E−60 | 0.103692029 | 0.217 | 0.063 | 4.82E−56 |
| Hist1h2bc | 1.96E−60 | −0.197841473 | 0.811 | 0.777 | 4.86E−56 |
| Mib1 | 2.14E−60 | 0.148818716 | 0.438 | 0.226 | 5.31E−56 |
| Ppp1r10 | 2.37E−60 | 0.113871639 | 0.292 | 0.115 | 5.89E−56 |
| Sik3 | 2.40E−60 | 0.113008636 | 0.391 | 0.192 | 5.96E−56 |
| Ptprj | 2.49E−60 | 0.122121887 | 0.294 | 0.115 | 6.18E−56 |
| Itch | 2.57E−60 | 0.151077267 | 0.399 | 0.195 | 6.37E−56 |
| Kif5b | 3.75E−60 | 0.150172912 | 0.53 | 0.303 | 9.31E−56 |
| Csde1 | 4.29E−60 | 0.209918785 | 0.797 | 0.577 | 1.07E−55 |
| Ptprk | 4.82E−60 | 0.158881882 | 0.354 | 0.156 | 1.20E−55 |
| Ero1lb | 5.45E−60 | 0.108914288 | 0.414 | 0.213 | 1.35E−55 |
| Rab6a | 6.68E−60 | 0.191200862 | 0.57 | 0.335 | 1.66E−55 |
| Apex1 | 7.33E−60 | 0.137727736 | 0.396 | 0.191 | 1.82E−55 |
| Cldn3 | 7.97E−60 | −0.24877302 | 0.273 | 0.355 | 1.98E−55 |
| Top1 | 9.07E−60 | 0.13764669 | 0.502 | 0.282 | 2.25E−55 |
| Rab3gap1 | 9.76E−60 | 0.112449261 | 0.348 | 0.159 | 2.42E−55 |
| Tspan4 | 1.12E−59 | 0.181956703 | 0.451 | 0.23 | 2.78E−55 |
| Hao1 | 1.23E−59 | 0.196088416 | 0.924 | 0.758 | 3.06E−55 |
| B4galt5 | 1.37E−59 | 0.112864448 | 0.229 | 0.07 | 3.41E−55 |
| Cln8 | 1.39E−59 | 0.104477829 | 0.314 | 0.133 | 3.46E−55 |
| Alkbh5 | 1.50E−59 | 0.198532353 | 0.507 | 0.279 | 3.73E−55 |
| Birc6 | 1.50E−59 | 0.164860409 | 0.502 | 0.28 | 3.74E−55 |
| 4931406C07Rik | 1.70E−59 | −0.233221778 | 0.789 | 0.748 | 4.22E−55 |
| Deb1 | 1.71E−59 | −0.129415477 | 0.515 | 0.443 | 4.25E−55 |
| Cyp3a59 | 2.07E−59 | −0.263118105 | 0.281 | 0.344 | 5.15E−55 |
| Mpc1 | 2.66E−59 | −0.205050162 | 0.349 | 0.369 | 6.61E−55 |
| Tnks2 | 3.54E−59 | 0.110931336 | 0.392 | 0.197 | 8.78E−55 |
| Ip6k2 | 3.86E−59 | 0.142196587 | 0.392 | 0.192 | 9.57E−55 |
| Derl2 | 5.00E−59 | −0.12950658 | 0.573 | 0.499 | 1.24E−54 |
| Zbtb41 | 6.03E−59 | 0.104784905 | 0.255 | 0.09 | 1.50E−54 |
| Acacb | 6.61E−59 | 0.172458783 | 0.33 | 0.137 | 1.64E−54 |
| Ccdc25 | 6.64E−59 | 0.153512231 | 0.493 | 0.271 | 1.65E−54 |
| Eif4g1 | 7.36E−59 | 0.201528669 | 0.865 | 0.669 | 1.83E−54 |
| Mob1b | 7.83E−59 | 0.134732265 | 0.407 | 0.203 | 1.94E−54 |
| Rab1 | 8.62E−59 | 0.211939923 | 0.673 | 0.438 | 2.14E−54 |
| Rad54l2 | 9.83E−59 | 0.101253342 | 0.251 | 0.092 | 2.44E−54 |
| Cyp3a16 | 1.47E−58 | −0.229013809 | 0.05 | 0.17 | 3.65E−54 |
| Ghr | 1.91E−58 | 0.197884255 | 0.963 | 0.833 | 4.74E−54 |
| Vgll4 | 1.98E−58 | 0.152050279 | 0.363 | 0.164 | 4.92E−54 |
| Rlim | 2.11E−58 | 0.106781063 | 0.323 | 0.142 | 5.23E−54 |
| Sdsl | 2.12E−58 | 0.128070508 | 0.321 | 0.137 | 5.27E−54 |
| Uqcrb | 2.16E−58 | −0.157541559 | 0.721 | 0.641 | 5.35E−54 |
| Psmb1 | 2.75E−58 | −0.18427868 | 0.822 | 0.759 | 6.83E−54 |
| Ebpl | 3.93E−58 | −0.155251391 | 0.48 | 0.451 | 9.75E−54 |
| Gnl2 | 4.20E−58 | 0.131907392 | 0.366 | 0.171 | 1.04E−53 |
| F11 | 4.61E−58 | 0.183078168 | 0.512 | 0.287 | 1.15E−53 |
| Map2k4 | 5.25E−58 | 0.119089109 | 0.36 | 0.17 | 1.30E−53 |
| Sat1 | 5.31E−58 | 0.13070979 | 0.36 | 0.167 | 1.32E−53 |
| Cpne3 | 5.59E−58 | 0.182285896 | 0.537 | 0.31 | 1.39E−53 |
| C8g | 6.46E−58 | −0.1570956 | 0.965 | 0.91 | 1.61E−53 |
| Arrdc4 | 7.07E−58 | 0.100382723 | 0.207 | 0.061 | 1.76E−53 |
| Cltc | 7.52E−58 | 0.238358233 | 0.647 | 0.414 | 1.87E−53 |
| Ptpn11 | 7.96E−58 | 0.122589174 | 0.418 | 0.218 | 1.98E−53 |
| Dio1 | 8.16E−58 | −0.238782365 | 0.544 | 0.542 | 2.03E−53 |
| Akr1c19 | 8.22E−58 | −0.179403044 | 0.547 | 0.527 | 2.04E−53 |
| Cct8 | 9.67E−58 | 0.156517607 | 0.674 | 0.445 | 2.40E−53 |
| Dpyd | 1.41E−57 | 0.207189814 | 0.857 | 0.659 | 3.51E−53 |
| 4833439L19Rik | 1.42E−57 | 0.107135389 | 0.341 | 0.158 | 3.53E−53 |
| Mrps18c | 1.45E−57 | −0.211258064 | 0.403 | 0.394 | 3.61E−53 |
| Mpzl2 | 1.66E−57 | 0.112096706 | 0.33 | 0.148 | 4.12E−53 |
| Masp1 | 1.68E−57 | 0.120716874 | 0.347 | 0.16 | 4.16E−53 |
| Tram1 | 1.72E−57 | 0.180903738 | 0.737 | 0.512 | 4.26E−53 |
| Uggt1 | 2.14E−57 | 0.147681553 | 0.408 | 0.204 | 5.32E−53 |
| Sec31a | 2.29E−57 | 0.151339369 | 0.53 | 0.31 | 5.68E−53 |
| Reep3 | 2.41E−57 | 0.201856133 | 0.654 | 0.422 | 5.99E−53 |
| Baz1b | 2.42E−57 | 0.121966091 | 0.416 | 0.215 | 6.02E−53 |
| Csnk2b | 2.44E−57 | 0.1035331 | 0.39 | 0.196 | 6.06E−53 |
| Hsdl2 | 2.60E−57 | 0.206098727 | 0.669 | 0.438 | 6.46E−53 |
| Eif4b | 3.03E−57 | 0.163112238 | 0.666 | 0.438 | 7.53E−53 |
| Tmem19 | 3.40E−57 | 0.214785487 | 0.693 | 0.465 | 8.45E−53 |
| Dpp9 | 3.50E−57 | 0.141994305 | 0.378 | 0.182 | 8.68E−53 |
| Erap1 | 3.86E−57 | 0.136126562 | 0.452 | 0.244 | 9.59E−53 |
| Sf3b1 | 3.86E−57 | 0.196390125 | 0.637 | 0.406 | 9.60E−53 |
| Gas5 | 3.98E−57 | −0.204205779 | 0.552 | 0.527 | 9.89E−53 |
| Gys2 | 4.07E−57 | 0.152105722 | 0.582 | 0.358 | 1.01E−52 |
| Atp8b1 | 4.27E−57 | 0.12732077 | 0.372 | 0.182 | 1.06E−52 |
| Impad1 | 4.33E−57 | 0.115657521 | 0.464 | 0.263 | 1.08E−52 |
| BCl2l13 | 4.37E−57 | 0.100287331 | 0.328 | 0.152 | 1.08E−52 |
| Rps15 | 5.66E−57 | −0.213338023 | 0.581 | 0.528 | 1.41E−52 |
| Mapk14 | 5.96E−57 | 0.137832043 | 0.44 | 0.232 | 1.48E−52 |
| Ogdh | 6.23E−57 | 0.220189108 | 0.601 | 0.37 | 1.55E−52 |
| Huwe1 | 7.13E−57 | 0.177152033 | 0.536 | 0.314 | 1.77E−52 |
| Slc25a1 | 8.37E−57 | 0.187178873 | 0.724 | 0.499 | 2.08E−52 |
| Dennd1b | 8.64E−57 | 0.104019094 | 0.285 | 0.117 | 2.14E−52 |
| Naa50 | 9.38E−57 | 0.187968549 | 0.586 | 0.358 | 2.33E−52 |
| Nab1 | 1.35E−56 | 0.151021868 | 0.533 | 0.315 | 3.36E−52 |
| Spag7 | 1.45E−56 | 0.101789122 | 0.331 | 0.151 | 3.60E−52 |
| F11r | 1.56E−56 | 0.135500375 | 0.421 | 0.216 | 3.88E−52 |
| Pah | 1.79E−56 | 0.196282042 | 0.983 | 0.891 | 4.44E−52 |
| Uhrf1bp1l | 2.27E−56 | 0.100980999 | 0.397 | 0.207 | 5.63E−52 |
| Slc30a9 | 2.34E−56 | 0.116028379 | 0.438 | 0.235 | 5.80E−52 |
| Psap | 2.57E−56 | 0.145158043 | 0.736 | 0.513 | 6.38E−52 |
| Nr0b2 | 2.67E−56 | −0.203060823 | 0.227 | 0.28 | 6.63E−52 |
| Ggcx | 2.98E−56 | 0.118127678 | 0.421 | 0.225 | 7.41E−52 |
| Itih1 | 3.10E−56 | 0.213777239 | 0.946 | 0.816 | 7.71E−52 |
| Pdzd8 | 3.45E−56 | 0.138689794 | 0.407 | 0.206 | 8.57E−52 |
| Ppp2r2d | 3.50E−56 | 0.110486018 | 0.45 | 0.247 | 8.70E−52 |
| Vwa8 | 3.52E−56 | 0.184402682 | 0.534 | 0.311 | 8.74E−52 |
| Mcl1 | 3.90E−56 | 0.206406082 | 0.601 | 0.371 | 9.67E−52 |
| Acadl | 4.72E−56 | 0.192143091 | 0.819 | 0.612 | 1.17E−51 |
| Cebpg | 4.86E−56 | 0.13869941 | 0.447 | 0.239 | 1.21E−51 |
| Trp53inp2 | 4.90E−56 | 0.176936721 | 0.655 | 0.428 | 1.22E−51 |
| Kpna1 | 5.49E−56 | 0.161541953 | 0.411 | 0.206 | 1.36E−51 |
| Ndufa4 | 6.92E−56 | −0.196233158 | 0.941 | 0.889 | 1.72E−51 |
| Chchd1 | 6.96E−56 | −0.178218308 | 0.469 | 0.438 | 1.73E−51 |
| Rhou | 6.96E−56 | 0.192074068 | 0.559 | 0.333 | 1.73E−51 |
| Pcca | 7.64E−56 | 0.150589178 | 0.594 | 0.373 | 1.90E−51 |
| Ap3m1 | 9.11E−56 | 0.133817705 | 0.408 | 0.211 | 2.26E−51 |
| Arl5a | 9.40E−56 | 0.169926786 | 0.508 | 0.289 | 2.33E−51 |
| Clcn4-2 | 1.11E−55 | 0.160606741 | 0.492 | 0.276 | 2.75E−51 |
| Ugt3a1 | 1.20E−55 | 0.100417636 | 0.243 | 0.088 | 2.97E−51 |
| Pank1 | 1.25E−55 | 0.222539616 | 0.873 | 0.704 | 3.11E−51 |
| Zfp110 | 1.53E−55 | 0.112121418 | 0.306 | 0.131 | 3.80E−51 |
| Eif4e2 | 2.21E−55 | 0.11430292 | 0.472 | 0.268 | 5.48E−51 |
| Rn7sk | 2.30E−55 | −0.149783999 | 0.242 | 0.186 | 5.70E−51 |
| Nufip2 | 2.32E−55 | 0.118078669 | 0.318 | 0.14 | 5.76E−51 |
| Phlda1 | 2.67E−55 | 0.201287476 | 0.583 | 0.356 | 6.64E−51 |
| Srsf6 | 2.84E−55 | 0.127543054 | 0.467 | 0.261 | 7.06E−51 |
| Ugt1a1 | 2.92E−55 | 0.183517826 | 0.502 | 0.283 | 7.25E−51 |
| Amfr | 2.93E−55 | 0.184512887 | 0.738 | 0.516 | 7.27E−51 |
| Nars | 2.97E−55 | 0.191856779 | 0.696 | 0.471 | 7.38E−51 |
| Paip1 | 3.87E−55 | 0.108346951 | 0.471 | 0.272 | 9.62E−51 |
| Hspa4 | 4.00E−55 | 0.171672026 | 0.728 | 0.506 | 9.94E−51 |
| Arf4 | 5.70E−55 | 0.159979368 | 0.58 | 0.36 | 1.42E−50 |
| Ubxn7 | 6.79E−55 | 0.100852418 | 0.267 | 0.107 | 1.69E−50 |
| Uap1 | 7.92E−55 | 0.104727103 | 0.298 | 0.126 | 1.97E−50 |
| Idh1 | 8.76E−55 | 0.208112098 | 0.921 | 0.766 | 2.18E−50 |
| Lysmd3 | 9.24E−55 | 0.119777827 | 0.352 | 0.166 | 2.29E−50 |
| Arih1 | 9.58E−55 | 0.113132381 | 0.408 | 0.216 | 2.38E−50 |
| Furin | 9.60E−55 | 0.191384876 | 0.643 | 0.417 | 2.38E−50 |
| Fam214a | 9.86E−55 | 0.133009262 | 0.448 | 0.248 | 2.45E−50 |
| 0610005C13Rik | 1.05E−54 | −0.2011928 | 0.677 | 0.653 | 2.60E−50 |
| Atp5a1 | 1.05E−54 | 0.174138529 | 0.984 | 0.895 | 2.60E−50 |
| Med13 | 1.07E−54 | 0.128372588 | 0.353 | 0.168 | 2.66E−50 |
| Ddx17 | 1.15E−54 | 0.159893824 | 0.491 | 0.277 | 2.85E−50 |
| Vps13d | 1.17E−54 | 0.117630793 | 0.367 | 0.181 | 2.91E−50 |
| Tmem30a | 1.18E−54 | 0.147452363 | 0.858 | 0.664 | 2.94E−50 |
| Glyr1 | 1.26E−54 | 0.139056743 | 0.476 | 0.269 | 3.14E−50 |
| Pank3 | 1.32E−54 | 0.128601194 | 0.451 | 0.251 | 3.28E−50 |
| Ranbp9 | 1.35E−54 | 0.114807175 | 0.35 | 0.169 | 3.36E−50 |
| St3gal4 | 1.41E−54 | 0.159434184 | 0.456 | 0.244 | 3.49E−50 |
| Etf1 | 1.44E−54 | 0.198764917 | 0.636 | 0.409 | 3.59E−50 |
| Tbc1d15 | 1.56E−54 | 0.1185677 | 0.443 | 0.244 | 3.88E−50 |
| Ndufb8 | 1.63E−54 | −0.158878768 | 0.79 | 0.746 | 4.05E−50 |
| Ttyh2 | 1.67E−54 | 0.113959235 | 0.362 | 0.179 | 4.14E−50 |
| 8430408G22Rik | 1.98E−54 | −0.221336883 | 0.336 | 0.392 | 4.93E−50 |
| Fam168b | 3.06E−54 | 0.106478831 | 0.292 | 0.124 | 7.59E−50 |
| Gdf15 | 3.33E−54 | 0.246982665 | 0.595 | 0.37 | 8.27E−50 |
| Gpd1 | 4.47E−54 | 0.200332863 | 0.856 | 0.665 | 1.11E−49 |
| Cgnl1 | 4.57E−54 | 0.139773771 | 0.44 | 0.238 | 1.13E−49 |
| Ndufs4 | 4.82E−54 | −0.100492283 | 0.662 | 0.562 | 1.20E−49 |
| Brd2 | 4.94E−54 | 0.258446853 | 0.629 | 0.408 | 1.23E−49 |
| Lclat1 | 4.98E−54 | 0.119397464 | 0.344 | 0.162 | 1.24E−49 |
| Dnajc5 | 5.50E−54 | 0.134563348 | 0.42 | 0.223 | 1.37E−49 |
| Tlcd2 | 5.97E−54 | −0.168255558 | 0.563 | 0.536 | 1.48E−49 |
| Mon2 | 6.36E−54 | 0.112969342 | 0.393 | 0.206 | 1.58E−49 |
| Calu | 7.05E−54 | 0.135275013 | 0.438 | 0.236 | 1.75E−49 |
| Mup14 | 7.18E−54 | −0.340890047 | 0.699 | 0.616 | 1.78E−49 |
| Onecut2 | 9.06E−54 | 0.133054586 | 0.53 | 0.323 | 2.25E−49 |
| Swi5 | 9.79E−54 | −0.115995539 | 0.572 | 0.501 | 2.43E−49 |
| Hopx | 1.01E−53 | 0.11914525 | 0.407 | 0.212 | 2.50E−49 |
| Gtf2b | 1.35E−53 | 0.140783181 | 0.383 | 0.189 | 3.35E−49 |
| Cebpb | 1.82E−53 | −0.114648972 | 0.548 | 0.45 | 4.53E−49 |
| Ubr5 | 1.96E−53 | 0.130354935 | 0.447 | 0.248 | 4.86E−49 |
| Prkd3 | 2.04E−53 | −0.146969299 | 0.538 | 0.49 | 5.07E−49 |
| Akap13 | 2.11E−53 | 0.147641979 | 0.416 | 0.218 | 5.25E−49 |
| Xpr1 | 2.30E−53 | 0.117363241 | 0.294 | 0.125 | 5.70E−49 |
| Adam9 | 2.79E−53 | 0.145575974 | 0.406 | 0.206 | 6.92E−49 |
| Hc | 3.23E−53 | 0.223732951 | 0.976 | 0.903 | 8.02E−49 |
| Atp5j2 | 3.89E−53 | −0.218750887 | 0.887 | 0.852 | 9.67E−49 |
| Ifitm2 | 3.99E−53 | 0.199025687 | 0.568 | 0.347 | 9.91E−49 |
| Bpnt1 | 4.01E−53 | 0.113495161 | 0.461 | 0.264 | 9.96E−49 |
| Cp | 6.52E−53 | 0.17916098 | 0.993 | 0.943 | 1.62E−48 |
| Rabgap1l | 6.94E−53 | 0.13415085 | 0.338 | 0.158 | 1.72E−48 |
| Adh6-ps1 | 8.20E−53 | −0.22400228 | 0.259 | 0.313 | 2.04E−48 |
| Aars | 8.79E−53 | 0.199299553 | 0.699 | 0.479 | 2.18E−48 |
| Slc30a10 | 8.80E−53 | 0.166623301 | 0.442 | 0.238 | 2.19E−48 |
| Eps15 | 1.07E−52 | 0.104532417 | 0.351 | 0.173 | 2.65E−48 |
| Nckap1 | 1.10E−52 | 0.109949167 | 0.552 | 0.349 | 2.72E−48 |
| Dap | 1.10E−52 | 0.20987441 | 0.842 | 0.654 | 2.72E−48 |
| Slc16a1 | 1.15E−52 | 0.136030092 | 0.383 | 0.191 | 2.85E−48 |
| Ubb | 1.22E−52 | −0.263773933 | 0.448 | 0.45 | 3.04E−48 |
| Zfand4 | 1.71E−52 | 0.121779444 | 0.309 | 0.136 | 4.25E−48 |
| Ireb2 | 1.72E−52 | 0.163596475 | 0.536 | 0.321 | 4.27E−48 |
| Ugdh | 1.95E−52 | 0.175644914 | 0.808 | 0.604 | 4.84E−48 |
| Gm24245 | 1.97E−52 | −0.254157233 | 0.192 | 0.214 | 4.90E−48 |
| Ces1g | 1.99E−52 | −0.129319624 | 0.627 | 0.569 | 4.93E−48 |
| Ywhab | 2.03E−52 | 0.186791118 | 0.656 | 0.434 | 5.04E−48 |
| Hgfac | 2.16E−52 | 0.181878452 | 0.66 | 0.438 | 5.38E−48 |
| Rangap1 | 2.23E−52 | 0.116918523 | 0.44 | 0.244 | 5.53E−48 |
| Purb | 2.27E−52 | 0.118154372 | 0.337 | 0.158 | 5.63E−48 |
| Cpeb4 | 2.49E−52 | 0.107417249 | 0.496 | 0.299 | 6.19E−48 |
| Dusp3 | 2.73E−52 | 0.121478974 | 0.415 | 0.221 | 6.78E−48 |
| Golph3 | 2.78E−52 | 0.111893985 | 0.415 | 0.223 | 6.90E−48 |
| Rpl35 | 2.79E−52 | −0.175144379 | 0.216 | 0.225 | 6.93E−48 |
| Prdx1 | 3.09E−52 | −0.174743472 | 0.971 | 0.927 | 7.67E−48 |
| Capzb | 3.11E−52 | 0.162969545 | 0.561 | 0.344 | 7.71E−48 |
| Parva | 3.29E−52 | 0.146537234 | 0.504 | 0.296 | 8.18E−48 |
| Nr5a2 | 3.59E−52 | 0.139283512 | 0.425 | 0.228 | 8.91E−48 |
| Pdcd6ip | 3.70E−52 | 0.181372134 | 0.556 | 0.338 | 9.18E−48 |
| Akr1c14 | 3.92E−52 | −0.173913844 | 0.758 | 0.707 | 9.73E−48 |
| Sppl2a | 4.25E−52 | 0.169493485 | 0.769 | 0.56 | 1.06E−47 |
| Gstm1 | 4.43E−52 | −0.221666006 | 0.984 | 0.935 | 1.10E−47 |
| As3mt | 5.14E−52 | 0.123667446 | 0.359 | 0.175 | 1.28E−47 |
| Atp5d | 5.35E−52 | 0.209976794 | 0.871 | 0.712 | 1.33E−47 |
| Ahcyl2 | 5.51E−52 | 0.103953438 | 0.231 | 0.084 | 1.37E−47 |
| Man1a | 5.66E−52 | 0.190591338 | 0.799 | 0.595 | 1.40E−47 |
| Atp11b | 5.99E−52 | 0.129558793 | 0.388 | 0.199 | 1.49E−47 |
| Arcn1 | 6.54E−52 | 0.158407448 | 0.582 | 0.366 | 1.62E−47 |
| Med1 | 6.78E−52 | 0.100895572 | 0.274 | 0.116 | 1.68E−47 |
| Narf | 6.98E−52 | 0.163638744 | 0.46 | 0.253 | 1.73E−47 |
| Chordc1 | 9.30E−52 | 0.116647052 | 0.391 | 0.204 | 2.31E−47 |
| Klkb1 | 1.08E−51 | −0.123984363 | 0.409 | 0.363 | 2.67E−47 |
| Ssr3 | 1.10E−51 | 0.214262703 | 0.796 | 0.596 | 2.74E−47 |
| Ubr4 | 1.11E−51 | 0.17740179 | 0.455 | 0.25 | 2.77E−47 |
| Tmf1 | 1.20E−51 | 0.127343798 | 0.512 | 0.307 | 2.98E−47 |
| Lin7c | 1.26E−51 | 0.133833672 | 0.384 | 0.194 | 3.12E−47 |
| Ifrd1 | 1.28E−51 | 0.175156039 | 0.379 | 0.185 | 3.17E−47 |
| Ppp6r3 | 1.29E−51 | 0.122373925 | 0.378 | 0.191 | 3.20E−47 |
| Sdc1 | 1.42E−51 | 0.202649914 | 0.686 | 0.469 | 3.52E−47 |
| Cd164 | 1.47E−51 | 0.139864927 | 0.749 | 0.542 | 3.66E−47 |
| Zfr | 1.55E−51 | 0.136139327 | 0.486 | 0.283 | 3.85E−47 |
| Sec23a | 1.59E−51 | 0.106870804 | 0.487 | 0.291 | 3.94E−47 |
| Rraga | 1.62E−51 | 0.117337603 | 0.383 | 0.196 | 4.03E−47 |
| Pxmp4 | 1.77E−51 | 0.233212684 | 0.731 | 0.526 | 4.40E−47 |
| Nlrp12 | 1.80E−51 | 0.153172692 | 0.648 | 0.43 | 4.46E−47 |
| Ythdf1 | 1.80E−51 | 0.102530375 | 0.317 | 0.147 | 4.48E−47 |
| Fam195a | 1.82E−51 | −0.190271304 | 0.355 | 0.373 | 4.52E−47 |
| Mier1 | 1.86E−51 | 0.10569398 | 0.409 | 0.224 | 4.61E−47 |
| Mrp63 | 2.10E−51 | −0.184375029 | 0.563 | 0.531 | 5.21E−47 |
| Bhmt2 | 2.27E−51 | −0.108586116 | 0.719 | 0.637 | 5.63E−47 |
| Usp33 | 2.40E−51 | 0.102734502 | 0.35 | 0.176 | 5.95E−47 |
| 1110008F13Rik | 2.78E−51 | −0.197564988 | 0.666 | 0.648 | 6.90E−47 |
| Plaa | 3.40E−51 | 0.113721305 | 0.383 | 0.198 | 8.45E−47 |
| 6-Mar | 3.70E−51 | 0.140127792 | 0.56 | 0.352 | 9.18E−47 |
| Uqcr11 | 4.09E−51 | −0.196760374 | 0.918 | 0.879 | 1.01E−46 |
| Celf1 | 4.96E−51 | 0.126385885 | 0.515 | 0.313 | 1.23E−46 |
| Lmbrd1 | 5.80E−51 | 0.113233603 | 0.474 | 0.279 | 1.44E−46 |
| Cat | 6.02E−51 | 0.151152692 | 0.998 | 0.971 | 1.50E−46 |
| Gm4952 | 6.05E−51 | −0.180740052 | 0.551 | 0.533 | 1.50E−46 |
| Iars | 6.26E−51 | 0.114030301 | 0.329 | 0.155 | 1.56E−46 |
| Gna12 | 6.93E−51 | −0.191090072 | 0.352 | 0.354 | 1.72E−46 |
| Sh3d19 | 7.12E−51 | 0.107931616 | 0.467 | 0.275 | 1.77E−46 |
| Hadhb | 7.55E−51 | −0.134029493 | 0.383 | 0.33 | 1.88E−46 |
| Mfn1 | 8.21E−51 | 0.11641477 | 0.353 | 0.175 | 2.04E−46 |
| Ppm1k | 8.28E−51 | −0.198117939 | 0.469 | 0.45 | 2.06E−46 |
| Pmpca | 8.29E−51 | 0.132846702 | 0.45 | 0.254 | 2.06E−46 |
| Hint1 | 8.36E−51 | −0.15108001 | 0.915 | 0.858 | 2.08E−46 |
| Api5 | 8.58E−51 | 0.12264057 | 0.358 | 0.176 | 2.13E−46 |
| Zzef1 | 9.17E−51 | 0.111336837 | 0.282 | 0.122 | 2.28E−46 |
| Cltb | 9.26E−51 | 0.111216553 | 0.533 | 0.333 | 2.30E−46 |
| Ssr1 | 1.00E−50 | 0.140942479 | 0.546 | 0.339 | 2.48E−46 |
| Vcpip1 | 1.17E−50 | 0.134850634 | 0.437 | 0.241 | 2.90E−46 |
| Slc35d1 | 1.46E−50 | 0.1666456 | 0.651 | 0.435 | 3.63E−46 |
| Sult1d1 | 1.70E−50 | −0.183796178 | 0.739 | 0.684 | 4.21E−46 |
| Bzw1 | 1.98E−50 | 0.162308789 | 0.558 | 0.344 | 4.93E−46 |
| Lats1 | 2.50E−50 | 0.101875138 | 0.317 | 0.149 | 6.22E−46 |
| Ces1e | 2.57E−50 | 0.166391723 | 0.779 | 0.576 | 6.37E−46 |
| Blvrb | 3.17E−50 | −0.243251643 | 0.562 | 0.564 | 7.88E−46 |
| Abca6 | 3.37E−50 | 0.198869605 | 0.719 | 0.507 | 8.37E−46 |
| Als2 | 3.42E−50 | 0.131619164 | 0.32 | 0.147 | 8.48E−46 |
| Rps18 | 3.43E−50 | −0.269206741 | 0.584 | 0.578 | 8.51E−46 |
| Cflar | 3.48E−50 | 0.110786697 | 0.421 | 0.231 | 8.63E−46 |
| Gss | 3.67E−50 | 0.141566962 | 0.557 | 0.346 | 9.10E−46 |
| Nudt4 | 3.71E−50 | 0.183172596 | 0.773 | 0.568 | 9.22E−46 |
| Dnm1l | 3.84E−50 | 0.110229955 | 0.414 | 0.227 | 9.53E−46 |
| Fabp2 | 3.96E−50 | −0.226921259 | 0.631 | 0.622 | 9.82E−46 |
| Pkp4 | 4.09E−50 | 0.102644807 | 0.445 | 0.258 | 1.02E−45 |
| Grlf1 | 4.50E−50 | 0.163008664 | 0.457 | 0.257 | 1.12E−45 |
| Wdr82 | 4.69E−50 | 0.105623725 | 0.313 | 0.144 | 1.17E−45 |
| Apol7a | 5.09E−50 | −0.165242629 | 0.39 | 0.382 | 1.26E−45 |
| Son | 5.49E−50 | 0.188921988 | 0.573 | 0.358 | 1.36E−45 |
| Lgals9 | 5.83E−50 | 0.184003378 | 0.936 | 0.793 | 1.45E−45 |
| Sec24d | 6.42E−50 | 0.138799708 | 0.447 | 0.251 | 1.60E−45 |
| Insr | 6.47E−50 | 0.133005386 | 0.495 | 0.295 | 1.61E−45 |
| Sel1l | 6.89E−50 | 0.100522207 | 0.586 | 0.392 | 1.71E−45 |
| Rtfdc1 | 7.59E−50 | 0.16242752 | 0.627 | 0.412 | 1.88E−45 |
| Hnrnpab | 8.17E−50 | 0.135931142 | 0.768 | 0.565 | 2.03E−45 |
| Itpr2 | 8.28E−50 | 0.100488718 | 0.353 | 0.179 | 2.06E−45 |
| Serbp1 | 1.03E−49 | 0.186271124 | 0.824 | 0.634 | 2.55E−45 |
| Dnaja3 | 1.31E−49 | 0.137126337 | 0.63 | 0.42 | 3.25E−45 |
| Tmem50a | 1.46E−49 | 0.122161634 | 0.533 | 0.33 | 3.62E−45 |
| Bnip2 | 1.64E−49 | 0.101515343 | 0.371 | 0.196 | 4.07E−45 |
| Osbpl9 | 1.68E−49 | 0.161985999 | 0.661 | 0.448 | 4.17E−45 |
| Uhmk1 | 1.72E−49 | 0.128955106 | 0.354 | 0.177 | 4.26E−45 |
| Hsd17b2 | 1.78E−49 | −0.143475762 | 0.775 | 0.711 | 4.42E−45 |
| Ttc36 | 1.82E−49 | −0.175280872 | 0.983 | 0.95 | 4.52E−45 |
| Pqlc1 | 1.93E−49 | −0.161639859 | 0.364 | 0.36 | 4.79E−45 |
| Setd2 | 2.06E−49 | 0.108533511 | 0.333 | 0.165 | 5.12E−45 |
| Ppp3r1 | 2.23E−49 | 0.111279553 | 0.309 | 0.142 | 5.55E−45 |
| Nek6 | 2.72E−49 | 0.136185787 | 0.436 | 0.242 | 6.76E−45 |
| Ugt2a3 | 2.99E−49 | −0.147859157 | 0.84 | 0.773 | 7.43E−45 |
| Zbtb20 | 3.04E−49 | 0.197963292 | 0.657 | 0.445 | 7.55E−45 |
| Eprs | 3.05E−49 | 0.19400896 | 0.7 | 0.488 | 7.57E−45 |
| A1cf | 3.13E−49 | 0.163393145 | 0.66 | 0.449 | 7.78E−45 |
| Rab18 | 4.02E−49 | 0.139929705 | 0.603 | 0.393 | 9.99E−45 |
| Dnajc11 | 4.18E−49 | 0.117471006 | 0.346 | 0.17 | 1.04E−44 |
| Ethe1 | 5.13E−49 | −0.13151581 | 0.532 | 0.49 | 1.27E−44 |
| Agt | 5.58E−49 | 0.169771558 | 0.941 | 0.817 | 1.39E−44 |
| S100a1 | 6.36E−49 | −0.11753536 | 0.75 | 0.661 | 1.58E−44 |
| Lrrc59 | 6.94E−49 | 0.163232541 | 0.484 | 0.28 | 1.72E−44 |
| Ubr2 | 6.97E−49 | 0.158348096 | 0.464 | 0.265 | 1.73E−44 |
| Ppp2r5e | 7.06E−49 | 0.107846882 | 0.445 | 0.259 | 1.75E−44 |
| Slain2 | 7.34E−49 | 0.115222374 | 0.354 | 0.179 | 1.82E−44 |
| Psma1 | 7.88E−49 | −0.105583275 | 0.606 | 0.521 | 1.96E−44 |
| Aco2 | 9.05E−49 | 0.155048742 | 0.702 | 0.493 | 2.25E−44 |
| Faah | 1.00E−48 | 0.139243096 | 0.623 | 0.415 | 2.48E−44 |
| Pten | 1.24E−48 | 0.166403783 | 0.669 | 0.458 | 3.07E−44 |
| Akr1c13 | 1.24E−48 | −0.11922584 | 0.517 | 0.457 | 3.09E−44 |
| Cbs | 1.27E−48 | 0.199528185 | 0.901 | 0.749 | 3.15E−44 |
| Slc25a5 | 1.29E−48 | −0.141114149 | 0.778 | 0.717 | 3.19E−44 |
| 2610507B11Rik | 1.29E−48 | 0.14984373 | 0.513 | 0.31 | 3.21E−44 |
| Slc38a10 | 1.30E−48 | 0.110122581 | 0.481 | 0.291 | 3.22E−44 |
| Ttc23 | 1.33E−48 | 0.111807305 | 0.375 | 0.199 | 3.30E−44 |
| Pdhb | 1.57E−48 | 0.129818342 | 0.67 | 0.468 | 3.89E−44 |
| Psma2 | 1.59E−48 | −0.123180488 | 0.607 | 0.538 | 3.95E−44 |
| Ociad1 | 1.69E−48 | 0.138322793 | 0.659 | 0.45 | 4.19E−44 |
| Dhcr24 | 1.81E−48 | 0.165813919 | 0.932 | 0.783 | 4.50E−44 |
| Car8 | 2.09E−48 | −0.101794556 | 0.539 | 0.465 | 5.19E−44 |
| Trim25 | 2.14E−48 | 0.126667357 | 0.421 | 0.233 | 5.31E−44 |
| Pygl | 2.32E−48 | 0.19507544 | 0.92 | 0.776 | 5.77E−44 |
| Prkar1a | 2.53E−48 | 0.165756705 | 0.67 | 0.46 | 6.28E−44 |
| Cxadr | 2.55E−48 | 0.144004472 | 0.545 | 0.345 | 6.32E−44 |
| Commd6 | 4.18E−48 | −0.168923514 | 0.391 | 0.39 | 1.04E−43 |
| Chchd2 | 4.90E−48 | −0.200916511 | 0.953 | 0.916 | 1.22E−43 |
| Cacul1 | 5.03E−48 | 0.127222559 | 0.423 | 0.236 | 1.25E−43 |
| Cyp2f2 | 5.05E−48 | 0.275177176 | 0.965 | 0.863 | 1.25E−43 |
| Nfe2l1 | 5.22E−48 | 0.132452495 | 0.497 | 0.303 | 1.30E−43 |
| Dennd5b | 5.95E−48 | 0.129106401 | 0.531 | 0.334 | 1.48E−43 |
| Klc4 | 6.82E−48 | 0.102388831 | 0.4 | 0.223 | 1.69E−43 |
| Glol | 6.86E−48 | −0.154642914 | 0.804 | 0.749 | 1.70E−43 |
| Cul4a | 7.75E−48 | 0.117942041 | 0.494 | 0.303 | 1.93E−43 |
| Cul1 | 7.81E−48 | 0.116195619 | 0.511 | 0.313 | 1.94E−43 |
| Atxn7l3b | 7.88E−48 | 0.191387966 | 0.686 | 0.476 | 1.96E−43 |
| Dnajc19 | 8.94E−48 | −0.156899631 | 0.412 | 0.387 | 2.22E−43 |
| Col18a1 | 9.46E−48 | 0.159648412 | 0.934 | 0.79 | 2.35E−43 |
| Tmem243 | 1.05E−47 | −0.137755038 | 0.613 | 0.557 | 2.61E−43 |
| Nudt12 | 1.13E−47 | 0.112919569 | 0.446 | 0.26 | 2.80E−43 |
| Metap2 | 1.14E−47 | 0.120185231 | 0.479 | 0.285 | 2.83E−43 |
| Rpl36al | 1.22E−47 | −0.186625637 | 0.601 | 0.551 | 3.02E−43 |
| 1300002K09Rik | 1.26E−47 | 0.134310334 | 0.713 | 0.514 | 3.12E−43 |
| F5 | 1.41E−47 | 0.202197412 | 0.886 | 0.728 | 3.50E−43 |
| Creld2 | 1.42E−47 | 0.138271923 | 0.494 | 0.296 | 3.53E−43 |
| Agpat2 | 1.57E−47 | −0.124813217 | 0.699 | 0.638 | 3.90E−43 |
| Tmem248 | 1.61E−47 | 0.118092361 | 0.428 | 0.242 | 3.99E−43 |
| Ppp2ca | 1.61E−47 | 0.154757155 | 0.64 | 0.43 | 4.00E−43 |
| Paics | 1.65E−47 | 0.198478345 | 0.842 | 0.662 | 4.10E−43 |
| Gspt1 | 1.72E−47 | 0.141363417 | 0.665 | 0.458 | 4.27E−43 |
| Prdx3 | 1.89E−47 | 0.134710601 | 0.504 | 0.305 | 4.69E−43 |
| Dnaja2 | 1.92E−47 | 0.160071794 | 0.612 | 0.402 | 4.77E−43 |
| Psma6 | 1.96E−47 | −0.114376366 | 0.765 | 0.675 | 4.87E−43 |
| Slco1a1 | 2.00E−47 | 0.134873793 | 0.927 | 0.777 | 4.97E−43 |
| Psma4 | 2.34E−47 | −0.153086391 | 0.544 | 0.495 | 5.82E−43 |
| Cyp2e1 | 2.61E−47 | −0.301497571 | 0.817 | 0.911 | 6.49E−43 |
| Gsta2 | 2.63E−47 | −0.188661153 | 0.296 | 0.333 | 6.53E−43 |
| Nrd1 | 3.09E−47 | 0.127908934 | 0.495 | 0.298 | 7.68E−43 |
| C1d | 3.10E−47 | −0.142249747 | 0.377 | 0.354 | 7.70E−43 |
| Vps37a | 3.47E−47 | 0.104057579 | 0.464 | 0.278 | 8.62E−43 |
| Nadk | 3.81E−47 | 0.15446959 | 0.661 | 0.454 | 9.47E−43 |
| Bckdha | 4.33E−47 | −0.144632079 | 0.693 | 0.63 | 1.08E−42 |
| 2-Sep | 4.81E−47 | 0.114840052 | 0.409 | 0.227 | 1.20E−42 |
| Mtpn | 5.14E−47 | 0.145939415 | 0.657 | 0.452 | 1.28E−42 |
| Fxr1 | 5.32E−47 | 0.101455713 | 0.421 | 0.241 | 1.32E−42 |
| Rabac1 | 5.83E−47 | −0.211715679 | 0.522 | 0.524 | 1.45E−42 |
| Osbpl8 | 7.65E−47 | 0.102268292 | 0.354 | 0.185 | 1.90E−42 |
| Slc31a1 | 8.37E−47 | 0.182041621 | 0.693 | 0.486 | 2.08E−42 |
| Pde4dip | 9.36E−47 | 0.135519348 | 0.442 | 0.251 | 2.32E−42 |
| Lamtor4 | 9.51E−47 | −0.133507304 | 0.42 | 0.372 | 2.36E−42 |
| Abce1 | 9.90E−47 | 0.132820596 | 0.506 | 0.31 | 2.46E−42 |
| Sco2 | 1.08E−46 | −0.198692618 | 0.507 | 0.508 | 2.68E−42 |
| Erp44 | 1.22E−46 | 0.108210732 | 0.525 | 0.336 | 3.03E−42 |
| Lman1 | 1.25E−46 | 0.147126353 | 0.838 | 0.655 | 3.12E−42 |
| Tm4sf4 | 1.42E−46 | 0.105750727 | 0.439 | 0.256 | 3.53E−42 |
| Hook1 | 1.44E−46 | 0.118548066 | 0.481 | 0.291 | 3.58E−42 |
| Osbp | 1.72E−46 | 0.106784104 | 0.323 | 0.158 | 4.27E−42 |
| Ninj1 | 1.74E−46 | −0.146496556 | 0.647 | 0.6 | 4.33E−42 |
| Yme1l1 | 1.75E−46 | 0.128865754 | 0.657 | 0.455 | 4.35E−42 |
| Gnas | 1.82E−46 | 0.159624116 | 0.947 | 0.82 | 4.52E−42 |
| Nampt | 1.96E−46 | 0.120285537 | 0.434 | 0.248 | 4.87E−42 |
| Pdha1 | 2.13E−46 | 0.133742662 | 0.675 | 0.474 | 5.28E−42 |
| AI182371 | 2.26E−46 | 0.179018804 | 0.81 | 0.622 | 5.62E−42 |
| Crem | 2.31E−46 | 0.101912709 | 0.392 | 0.218 | 5.74E−42 |
| Mrpl41 | 2.54E−46 | −0.172436199 | 0.341 | 0.348 | 6.30E−42 |
| Timm13 | 2.82E−46 | −0.204422177 | 0.695 | 0.671 | 7.01E−42 |
| Fyttd1 | 3.01E−46 | 0.101452644 | 0.354 | 0.186 | 7.47E−42 |
| Becn1 | 3.03E−46 | 0.126325957 | 0.382 | 0.204 | 7.51E−42 |
| Khk | 3.09E−46 | 0.101177619 | 0.976 | 0.863 | 7.67E−42 |
| Gnai3 | 3.18E−46 | 0.126569862 | 0.559 | 0.363 | 7.91E−42 |
| Gchfr | 3.33E−46 | −0.152643272 | 0.833 | 0.768 | 8.26E−42 |
| Mapk1 | 3.43E−46 | 0.120232033 | 0.453 | 0.27 | 8.53E−42 |
| Gyk | 3.49E−46 | 0.150378816 | 0.553 | 0.35 | 8.66E−42 |
| Ywhae | 3.56E−46 | 0.148553311 | 0.758 | 0.56 | 8.85E−42 |
| Ppp1r15b | 4.10E−46 | 0.120958229 | 0.48 | 0.291 | 1.02E−41 |
| Ctnna1 | 4.17E−46 | 0.12068953 | 0.588 | 0.391 | 1.04E−41 |
| Gckr | 4.43E−46 | 0.141702795 | 0.505 | 0.307 | 1.10E−41 |
| Sbfm1 | 4.65E−46 | −0.132167436 | 0.544 | 0.484 | 1.15E−41 |
| Etnk1 | 4.96E−46 | 0.15045907 | 0.527 | 0.328 | 1.23E−41 |
| Trap1 | 6.48E−46 | 0.101198423 | 0.731 | 0.541 | 1.61E−41 |
| Ndufs6 | 6.75E−46 | −0.195648216 | 0.771 | 0.724 | 1.68E−41 |
| Crot | 7.15E−46 | −0.119666122 | 0.988 | 0.942 | 1.77E−41 |
| Mbnl1 | 7.21E−46 | 0.103083775 | 0.48 | 0.297 | 1.79E−41 |
| Aldoa | 7.94E−46 | 0.122026615 | 0.511 | 0.316 | 1.97E−41 |
| Copb1 | 8.11E−46 | 0.132958439 | 0.628 | 0.429 | 2.01E−41 |
| AW112010 | 8.57E−46 | −0.128497272 | 0.473 | 0.435 | 2.13E−41 |
| Sptbn1 | 8.60E−46 | 0.11420622 | 0.445 | 0.26 | 2.14E−41 |
| Cxcl12 | 1.11E−45 | −0.119856412 | 0.483 | 0.419 | 2.77E−41 |
| Zfp106 | 1.17E−45 | 0.116608539 | 0.437 | 0.254 | 2.91E−41 |
| 9430023L20Rik | 1.20E−45 | −0.164422856 | 0.264 | 0.282 | 2.99E−41 |
| C1rl | 1.24E−45 | 0.10472398 | 0.601 | 0.404 | 3.09E−41 |
| Secisbp2l | 1.36E−45 | 0.117268869 | 0.506 | 0.317 | 3.37E−41 |
| 5-Mar | 1.37E−45 | 0.113105187 | 0.508 | 0.317 | 3.40E−41 |
| Golga4 | 1.44E−45 | 0.120034328 | 0.5 | 0.31 | 3.56E−41 |
| Ccdc50 | 1.54E−45 | 0.117452166 | 0.428 | 0.246 | 3.83E−41 |
| Map1lc3b | 1.57E−45 | 0.167697241 | 0.602 | 0.395 | 3.90E−41 |
| Pigyl | 1.75E−45 | −0.14064376 | 0.312 | 0.3 | 4.34E−41 |
| Cd81 | 1.93E−45 | −0.10012454 | 0.764 | 0.675 | 4.80E−41 |
| Clmn | 2.12E−45 | 0.159639031 | 0.509 | 0.31 | 5.25E−41 |
| Tsc22d1 | 2.25E−45 | −0.235196773 | 0.158 | 0.242 | 5.58E−41 |
| Ap1g1 | 2.57E−45 | 0.111599757 | 0.431 | 0.252 | 6.39E−41 |
| Usp32 | 2.65E−45 | 0.103466162 | 0.28 | 0.127 | 6.57E−41 |
| Bola1 | 2.65E−45 | −0.12138055 | 0.347 | 0.307 | 6.59E−41 |
| Xiap | 2.90E−45 | 0.11609823 | 0.575 | 0.378 | 7.20E−41 |
| Edem1 | 2.95E−45 | 0.172255296 | 0.635 | 0.429 | 7.33E−41 |
| Stat1 | 3.53E−45 | 0.130244409 | 0.353 | 0.18 | 8.76E−41 |
| Hdac11 | 3.81E−45 | −0.196507741 | 0.251 | 0.285 | 9.47E−41 |
| Fah | 3.98E−45 | 0.188483379 | 0.941 | 0.834 | 9.89E−41 |
| Acad11 | 4.08E−45 | 0.148432334 | 0.759 | 0.564 | 1.01E−40 |
| Uso1 | 4.50E−45 | 0.125106881 | 0.634 | 0.434 | 1.12E−40 |
| Tacc1 | 5.30E−45 | 0.10371879 | 0.339 | 0.171 | 1.32E−40 |
| St6gal1 | 5.67E−45 | 0.12698386 | 0.405 | 0.226 | 1.41E−40 |
| Tkt | 5.71E−45 | 0.104051911 | 0.615 | 0.424 | 1.42E−40 |
| Sar1a | 5.99E−45 | 0.138437968 | 0.541 | 0.343 | 1.49E−40 |
| Adh4 | 6.51E−45 | −0.13090615 | 0.571 | 0.521 | 1.62E−40 |
| Pbld1 | 6.67E−45 | −0.1818338 | 0.592 | 0.565 | 1.66E−40 |
| Crp | 6.74E−45 | 0.178468102 | 0.905 | 0.756 | 1.67E−40 |
| Spcs1 | 7.39E−45 | −0.141150139 | 0.446 | 0.413 | 1.84E−40 |
| Tgoln1 | 7.77E−45 | 0.176355766 | 0.768 | 0.575 | 1.93E−40 |
| Erf3b | 7.78E−45 | 0.128303984 | 0.563 | 0.366 | 1.93E−40 |
| Gstt3 | 7.97E−45 | −0.159722425 | 0.296 | 0.286 | 1.98E−40 |
| Gstm4 | 8.06E−45 | −0.157553603 | 0.478 | 0.444 | 2.00E−40 |
| Neat1 | 9.67E−45 | 0.164921803 | 0.293 | 0.135 | 2.40E−40 |
| Rps19 | 1.08E−44 | −0.151004347 | 0.562 | 0.474 | 2.69E−40 |
| Ndufb2 | 1.15E−44 | −0.154329774 | 0.685 | 0.629 | 2.86E−40 |
| Ndufa2 | 1.15E−44 | −0.194549142 | 0.792 | 0.738 | 2.86E−40 |
| Cyp4f15 | 1.21E−44 | −0.136129684 | 0.486 | 0.428 | 3.00E−40 |
| Anp32a | 1.28E−44 | −0.154984115 | 0.436 | 0.413 | 3.17E−40 |
| Rps15a | 1.29E−44 | −0.139214885 | 0.337 | 0.296 | 3.21E−40 |
| Tmed7 | 1.30E−44 | 0.118885811 | 0.557 | 0.367 | 3.22E−40 |
| Gfm1 | 1.32E−44 | 0.132118047 | 0.525 | 0.332 | 3.28E−40 |
| Psme2 | 1.47E−44 | −0.120525793 | 0.315 | 0.276 | 3.64E−40 |
| Cyp3a44 | 1.56E−44 | −0.205800676 | 0.164 | 0.183 | 3.87E−40 |
| Nfia | 1.59E−44 | 0.132814201 | 0.43 | 0.245 | 3.95E−40 |
| Prrc2c | 1.86E−44 | 0.108378548 | 0.369 | 0.201 | 4.62E−40 |
| Surf4 | 1.87E−44 | 0.150111361 | 0.746 | 0.551 | 4.64E−40 |
| 1110058L19Rik | 2.30E−44 | −0.160260673 | 0.272 | 0.281 | 5.71E−40 |
| Hspa9 | 2.77E−44 | 0.158702904 | 0.947 | 0.824 | 6.88E−40 |
| Vamp8 | 2.81E−44 | −0.111795351 | 0.713 | 0.636 | 6.98E−40 |
| Sdha | 2.85E−44 | 0.152056738 | 0.902 | 0.747 | 7.08E−40 |
| Proz | 2.91E−44 | 0.135145053 | 0.724 | 0.53 | 7.22E−40 |
| Brk1 | 2.94E−44 | −0.119149676 | 0.393 | 0.349 | 7.31E−40 |
| Mrps21 | 2.99E−44 | −0.121789459 | 0.257 | 0.215 | 7.43E−40 |
| Rnase4 | 3.02E−44 | 0.221993349 | 0.986 | 0.921 | 7.49E−40 |
| Ccs | 3.19E−44 | −0.107043033 | 0.754 | 0.676 | 7.91E−40 |
| Trip12 | 3.24E−44 | 0.158568104 | 0.544 | 0.345 | 8.06E−40 |
| Acadsb | 3.49E−44 | 0.161705277 | 0.79 | 0.601 | 8.66E−40 |
| Dlg1 | 3.65E−44 | 0.120208184 | 0.38 | 0.208 | 9.05E−40 |
| Rpl34 | 3.76E−44 | −0.130799019 | 0.465 | 0.398 | 9.34E−40 |
| Nr1h4 | 5.00E−44 | 0.142664592 | 0.634 | 0.434 | 1.24E−39 |
| Qprt | 5.29E−44 | 0.156064415 | 0.698 | 0.498 | 1.31E−39 |
| Spop | 7.17E−44 | 0.114116164 | 0.59 | 0.394 | 1.78E−39 |
| Fdft1 | 7.30E−44 | −0.11322184 | 0.369 | 0.335 | 1.81E−39 |
| Chd4 | 7.40E−44 | −0.114509098 | 0.484 | 0.39 | 1.84E−39 |
| Afm | 7.47E−44 | 0.159606308 | 0.745 | 0.55 | 1.86E−39 |
| Dhx15 | 9.21E−44 | 0.107938501 | 0.456 | 0.276 | 2.29E−39 |
| Afg3l2 | 9.25E−44 | 0.110518283 | 0.491 | 0.308 | 2.30E−39 |
| Ccnd1 | 9.27E−44 | 0.130127323 | 0.144 | 0.035 | 2.30E−39 |
| Iigp1 | 9.65E−44 | 0.232002466 | 0.895 | 0.759 | 2.40E−39 |
| 2810007J24Rik | 9.98E−44 | −0.118701956 | 0.988 | 0.943 | 2.48E−39 |
| Spcs3 | 1.06E−43 | 0.129856927 | 0.472 | 0.283 | 2.63E−39 |
| Psmc5 | 1.09E−43 | 0.135453234 | 0.64 | 0.441 | 2.70E−39 |
| Cyp4a10 | 1.35E−43 | −0.289941035 | 0.614 | 0.657 | 3.36E−39 |
| Fbxo21 | 1.42E−43 | −0.200640929 | 0.249 | 0.298 | 3.53E−39 |
| Gm5424 | 1.63E−43 | −0.137211896 | 0.345 | 0.3 | 4.05E−39 |
| Hrsp12 | 1.75E−43 | −0.156961195 | 0.98 | 0.939 | 4.33E−39 |
| H2-D1 | 1.78E−43 | 0.167536811 | 0.916 | 0.774 | 4.42E−39 |
| Slc35a3 | 1.90E−43 | 0.117116355 | 0.424 | 0.246 | 4.71E−39 |
| Cpn1 | 1.94E−43 | 0.146334172 | 0.84 | 0.665 | 4.82E−39 |
| Dpp4 | 2.07E−43 | 0.102369468 | 0.583 | 0.394 | 5.14E−39 |
| Chdh | 2.26E−43 | 0.1451577 | 0.587 | 0.392 | 5.62E−39 |
| Bche | 2.49E−43 | 0.162843566 | 0.566 | 0.369 | 6.18E−39 |
| Gas2 | 2.51E−43 | 0.142777263 | 0.55 | 0.358 | 6.24E−39 |
| Slc22a1 | 2.62E−43 | −0.199196167 | 0.777 | 0.747 | 6.51E−39 |
| Tpp2 | 2.79E−43 | 0.101764106 | 0.354 | 0.19 | 6.93E−39 |
| Skp1a | 2.88E−43 | 0.172529381 | 0.791 | 0.606 | 7.14E−39 |
| Got2 | 3.06E−43 | 0.104352218 | 0.823 | 0.644 | 7.60E−39 |
| G3bp2 | 3.51E−43 | 0.126428906 | 0.551 | 0.359 | 8.71E−39 |
| Rps8 | 3.56E−43 | −0.118925202 | 0.545 | 0.471 | 8.84E−39 |
| Usp24 | 3.81E−43 | 0.11323894 | 0.436 | 0.255 | 9.46E−39 |
| Csnk1a1 | 3.98E−43 | 0.136356879 | 0.64 | 0.443 | 9.89E−39 |
| Slc20a2 | 4.01E−43 | 0.110778719 | 0.383 | 0.215 | 9.96E−39 |
| Igf2r | 4.28E−43 | 0.102105026 | 0.32 | 0.164 | 1.06E−38 |
| Dazap2 | 4.28E−43 | −0.101392856 | 0.536 | 0.472 | 1.06E−38 |
| Rrp1 | 5.42E−43 | 0.103292166 | 0.511 | 0.327 | 1.35E−38 |
| Gucd1 | 5.48E−43 | −0.1039149 | 0.581 | 0.511 | 1.36E−38 |
| Tspo | 5.70E−43 | −0.171115456 | 0.532 | 0.506 | 1.42E−38 |
| Pgrmc1 | 5.75E−43 | 0.159063843 | 0.99 | 0.94 | 1.43E−38 |
| Vps35 | 7.21E−43 | 0.105568566 | 0.509 | 0.329 | 1.79E−38 |
| Stt3b | 7.63E−43 | 0.102410897 | 0.518 | 0.335 | 1.90E−38 |
| Ssu72 | 7.77E−43 | −0.112771136 | 0.553 | 0.487 | 1.93E−38 |
| Mmadhc | 8.60E−43 | 0.108169821 | 0.471 | 0.288 | 2.13E−38 |
| Cyp3a13 | 8.99E−43 | 0.148503232 | 0.759 | 0.569 | 2.23E−38 |
| Chp1 | 9.73E−43 | 0.142681268 | 0.709 | 0.515 | 2.42E−38 |
| Cyp2c44 | 9.92E−43 | 0.164710905 | 0.863 | 0.698 | 2.46E−38 |
| Adi1 | 1.04E−42 | 0.117291753 | 0.611 | 0.419 | 2.59E−38 |
| Aco1 | 1.14E−42 | 0.123631223 | 0.714 | 0.525 | 2.83E−38 |
| Gng12 | 1.16E−42 | 0.103486756 | 0.44 | 0.265 | 2.88E−38 |
| Akr1a1 | 1.27E−42 | −0.118556816 | 0.919 | 0.857 | 3.16E−38 |
| Prdx5 | 1.31E−42 | −0.145867251 | 0.878 | 0.834 | 3.26E−38 |
| Pex16 | 1.59E−42 | −0.131815455 | 0.466 | 0.416 | 3.94E−38 |
| Arhgap5 | 1.80E−42 | 0.146393752 | 0.651 | 0.454 | 4.47E−38 |
| 1810022K09Rik | 1.90E−42 | −0.180096346 | 0.286 | 0.301 | 4.71E−38 |
| 1600014C10Rik | 2.28E−42 | −0.120294622 | 0.517 | 0.462 | 5.66E−38 |
| Slc39a4 | 2.34E−42 | 0.13395424 | 0.479 | 0.293 | 5.81E−38 |
| Sirt3 | 3.06E−42 | −0.139298161 | 0.45 | 0.413 | 7.61E−38 |
| 2810428I15Rik | 3.07E−42 | −0.101899359 | 0.418 | 0.349 | 7.62E−38 |
| Krtcap2 | 3.08E−42 | 0.101091751 | 0.488 | 0.308 | 7.65E−38 |
| Abca3 | 3.23E−42 | 0.102981662 | 0.338 | 0.179 | 8.03E−38 |
| Samm50 | 3.43E−42 | 0.131598944 | 0.534 | 0.341 | 8.51E−38 |
| Tax1bp1 | 3.59E−42 | 0.11382647 | 0.784 | 0.601 | 8.93E−38 |
| Klf9 | 3.63E−42 | 0.100500247 | 0.709 | 0.52 | 9.01E−38 |
| Fam120a | 4.13E−42 | 0.130340244 | 0.582 | 0.391 | 1.03E−37 |
| Sidt2 | 4.22E−42 | 0.110437187 | 0.395 | 0.223 | 1.05E−37 |
| Sec16a | 4.49E−42 | 0.125833914 | 0.382 | 0.211 | 1.11E−37 |
| Akr1d1 | 4.73E−42 | −0.121611839 | 0.563 | 0.515 | 1.17E−37 |
| Kcnk5 | 5.02E−42 | −0.168374452 | 0.107 | 0.173 | 1.25E−37 |
| Cab39l | 5.15E−42 | 0.116519219 | 0.515 | 0.331 | 1.28E−37 |
| D10Jhu81e | 5.21E−42 | −0.132961769 | 0.618 | 0.564 | 1.29E−37 |
| Shmt2 | 5.37E−42 | −0.116891554 | 0.533 | 0.465 | 1.33E−37 |
| Selenbp1 | 5.64E−42 | −0.185040969 | 0.725 | 0.699 | 1.40E−37 |
| Srp72 | 6.51E−42 | 0.123323755 | 0.651 | 0.461 | 1.62E−37 |
| Car5a | 6.86E−42 | −0.117324599 | 0.371 | 0.331 | 1.70E−37 |
| Snrpd2 | 7.08E−42 | −0.105764256 | 0.292 | 0.236 | 1.76E−37 |
| Rpl3 | 7.86E−42 | −0.104783236 | 0.402 | 0.332 | 1.95E−37 |
| Sec61a1 | 8.15E−42 | 0.141299735 | 0.67 | 0.476 | 2.02E−37 |
| Scarb1 | 8.93E−42 | 0.134417249 | 0.549 | 0.358 | 2.22E−37 |
| Rbpms | 9.27E−42 | 0.105037375 | 0.389 | 0.222 | 2.30E−37 |
| Psmb3 | 9.93E−42 | −0.12442445 | 0.398 | 0.345 | 2.47E−37 |
| Fkbp3 | 1.11E−41 | −0.113911562 | 0.366 | 0.323 | 2.75E−37 |
| Cox6a1 | 1.30E−41 | −0.131489256 | 0.939 | 0.887 | 3.23E−37 |
| Fetub | 1.39E−41 | 0.174405959 | 0.859 | 0.703 | 3.46E−37 |
| Slc25a44 | 1.50E−41 | 0.10239118 | 0.397 | 0.228 | 3.72E−37 |
| Tor1aip2 | 1.59E−41 | 0.114296102 | 0.573 | 0.386 | 3.94E−37 |
| Adk | 1.76E−41 | 0.127467378 | 0.967 | 0.86 | 4.37E−37 |
| Gpr146 | 2.17E−41 | 0.107393278 | 0.482 | 0.305 | 5.40E−37 |
| Rxra | 2.21E−41 | 0.116073514 | 0.759 | 0.574 | 5.50E−37 |
| Cox8a | 2.28E−41 | 0.176195798 | 0.796 | 0.621 | 5.66E−37 |
| Cct6a | 2.40E−41 | 0.104052442 | 0.586 | 0.399 | 5.95E−37 |
| Anpep | 2.60E−41 | 0.100416126 | 0.32 | 0.166 | 6.47E−37 |
| Ei24 | 3.14E−41 | 0.127457502 | 0.672 | 0.48 | 7.79E−37 |
| Mrpl20 | 3.63E−41 | −0.109230853 | 0.565 | 0.489 | 9.02E−37 |
| Serping1 | 3.66E−41 | 0.153322882 | 0.963 | 0.87 | 9.09E−37 |
| Srp9 | 3.97E−41 | −0.160772329 | 0.333 | 0.336 | 9.85E−37 |
| Tpr | 4.36E−41 | 0.117035633 | 0.521 | 0.339 | 1.08E−36 |
| Sos1 | 4.75E−41 | 0.105944408 | 0.385 | 0.221 | 1.18E−36 |
| Cox7a2 | 5.09E−41 | −0.139135338 | 0.858 | 0.784 | 1.26E−36 |
| Tm9sf3 | 5.60E−41 | 0.118956421 | 0.765 | 0.582 | 1.39E−36 |
| Lyrm5 | 5.81E−41 | −0.128193124 | 0.422 | 0.396 | 1.44E−36 |
| Sucla2 | 5.85E−41 | 0.115777694 | 0.727 | 0.544 | 1.45E−36 |
| 2410015M20Rik | 5.99E−41 | −0.169256363 | 0.635 | 0.604 | 1.49E−36 |
| Gstt2 | 6.55E−41 | −0.190732451 | 0.397 | 0.404 | 1.63E−36 |
| Cdk2ap2 | 7.14E−41 | −0.131121465 | 0.273 | 0.272 | 1.77E−36 |
| Pglyrp2 | 7.14E−41 | 0.150653064 | 0.607 | 0.414 | 1.77E−36 |
| Apof | 7.15E−41 | 0.13917521 | 0.98 | 0.897 | 1.78E−36 |
| Hsd17b7 | 7.52E−41 | 0.132910173 | 0.538 | 0.353 | 1.87E−36 |
| Mug2 | 9.79E−41 | 0.179746266 | 0.887 | 0.738 | 2.43E−36 |
| Rpl22 | 9.89E−41 | −0.10259468 | 0.556 | 0.47 | 2.46E−36 |
| Rpl32 | 1.11E−40 | −0.190948597 | 0.8 | 0.757 | 2.75E−36 |
| Sptan1 | 1.11E−40 | 0.102152857 | 0.37 | 0.21 | 2.76E−36 |
| Selt | 1.12E−40 | 0.141954072 | 0.694 | 0.503 | 2.77E−36 |
| Psmb6 | 1.12E−40 | −0.131951208 | 0.747 | 0.683 | 2.78E−36 |
| Ghitm | 1.13E−40 | 0.129508966 | 0.891 | 0.737 | 2.80E−36 |
| Rps10 | 1.16E−40 | −0.13253262 | 0.518 | 0.453 | 2.87E−36 |
| Copb2 | 1.67E−40 | 0.122594343 | 0.661 | 0.471 | 4.16E−36 |
| Dnajb9 | 1.69E−40 | 0.120160928 | 0.416 | 0.241 | 4.19E−36 |
| Wnk1 | 2.04E−40 | 0.10779068 | 0.392 | 0.227 | 5.08E−36 |
| Cyr61 | 2.07E−40 | 0.122951564 | 0.242 | 0.102 | 5.14E−36 |
| Rnaseh2c | 2.14E−40 | −0.112974422 | 0.445 | 0.387 | 5.30E−36 |
| Pltp | 2.46E−40 | 0.112739945 | 0.156 | 0.045 | 6.10E−36 |
| Abcf1 | 3.56E−40 | 0.117230118 | 0.501 | 0.315 | 8.83E−36 |
| PrpfS | 3.62E−40 | 0.112914767 | 0.505 | 0.331 | 8.98E−36 |
| Tmem106b | 3.79E−40 | 0.153891421 | 0.743 | 0.557 | 9.40E−36 |
| Eva1a | 3.89E−40 | −0.125757849 | 0.528 | 0.47 | 9.67E−36 |
| Hmgcl | 4.81E−40 | −0.133535955 | 0.793 | 0.729 | 1.19E−35 |
| Tomm70a | 4.81E−40 | 0.151256378 | 0.685 | 0.494 | 1.20E−35 |
| Mettl20 | 6.82E−40 | −0.163343248 | 0.311 | 0.327 | 1.69E−35 |
| Hibch | 9.07E−40 | −0.123582904 | 0.455 | 0.407 | 2.25E−35 |
| Sacm1l | 9.38E−40 | 0.102054913 | 0.457 | 0.29 | 2.33E−35 |
| Itgb1 | 1.10E−39 | 0.121718186 | 0.651 | 0.464 | 2.73E−35 |
| Lactb | 1.22E−39 | −0.118616118 | 0.454 | 0.403 | 3.03E−35 |
| Gadd45b | 1.23E−39 | −0.219990566 | 0.254 | 0.291 | 3.07E−35 |
| Eif5 | 1.26E−39 | −0.133564962 | 0.812 | 0.74 | 3.14E−35 |
| S100a10 | 1.29E−39 | −0.177702028 | 0.676 | 0.662 | 3.20E−35 |
| Ptprd | 1.41E−39 | 0.125532384 | 0.605 | 0.42 | 3.51E−35 |
| Chuk | 1.66E−39 | 0.125058676 | 0.607 | 0.421 | 4.11E−35 |
| Mtbfd1 | 1.75E−39 | 0.10691059 | 0.722 | 0.546 | 4.35E−35 |
| Abcb11 | 1.83E−39 | 0.127231815 | 0.926 | 0.791 | 4.55E−35 |
| Etfb | 2.14E−39 | −0.13767417 | 0.977 | 0.94 | 5.31E−35 |
| Hspe1 | 2.30E−39 | −0.157908691 | 0.6 | 0.557 | 5.72E−35 |
| Rpl23 | 2.35E−39 | −0.141561405 | 0.453 | 0.403 | 5.84E−35 |
| Htatip2 | 2.57E−39 | −0.152544453 | 0.334 | 0.329 | 6.38E−35 |
| 1110001A16Rik | 3.27E−39 | −0.11299875 | 0.212 | 0.208 | 8.12E−35 |
| Sqrdl | 3.48E−39 | 0.125432563 | 0.674 | 0.488 | 8.64E−35 |
| Rbp4 | 3.69E−39 | −0.134223218 | 1 | 0.999 | 9.15E−35 |
| Crls1 | 3.80E−39 | −0.102433805 | 0.288 | 0.263 | 9.45E−35 |
| Echs1 | 3.85E−39 | −0.101419752 | 0.89 | 0.817 | 9.55E−35 |
| Sqstm1 | 4.10E−39 | 0.16784685 | 0.923 | 0.805 | 1.02E−34 |
| Ndufb3 | 4.24E−39 | −0.145234718 | 0.47 | 0.43 | 1.05E−34 |
| Atp11c | 4.50E−39 | 0.107408002 | 0.76 | 0.587 | 1.12E−34 |
| Pitpnb | 4.52E−39 | 0.101346214 | 0.568 | 0.393 | 1.12E−34 |
| Dlst | 5.28E−39 | 0.119678303 | 0.694 | 0.512 | 1.31E−34 |
| Rrbp1 | 5.94E−39 | 0.147797245 | 0.904 | 0.768 | 1.47E−34 |
| Ndufa7 | 6.00E−39 | −0.18650272 | 0.739 | 0.718 | 1.49E−34 |
| Pcx | 7.57E−39 | 0.127735069 | 0.828 | 0.661 | 1.88E−34 |
| Ubr3 | 1.07E−38 | 0.107986329 | 0.587 | 0.41 | 2.65E−34 |
| Canx | 1.12E−38 | 0.163661783 | 0.941 | 0.835 | 2.79E−34 |
| Mlxipl | 1.54E−38 | −0.103437041 | 0.354 | 0.298 | 3.82E−34 |
| Slc9a3r1 | 1.55E−38 | 0.120027637 | 0.668 | 0.483 | 3.85E−34 |
| Mrpl42 | 1.70E−38 | −0.126024897 | 0.412 | 0.386 | 4.23E−34 |
| Gdi2 | 1.73E−38 | 0.15496797 | 0.782 | 0.605 | 4.30E−34 |
| Sphk2 | 2.01E−38 | −0.107275977 | 0.209 | 0.188 | 4.99E−34 |
| Sucnr1 | 2.78E−38 | −0.171877738 | 0.101 | 0.201 | 6.91E−34 |
| Cyp4f14 | 3.07E−38 | 0.122432234 | 0.773 | 0.6 | 7.63E−34 |
| Rab7 | 4.15E−38 | 0.115589878 | 0.583 | 0.402 | 1.03E−33 |
| Zfyve21 | 5.89E−38 | −0.129446613 | 0.227 | 0.245 | 1.46E−33 |
| 1500017E21Rik | 6.12E−38 | −0.116910818 | 0.52 | 0.476 | 1.52E−33 |
| Pfdn5 | 6.30E−38 | −0.104229394 | 0.579 | 0.511 | 1.57E−33 |
| C130074G19Rik | 6.57E−38 | −0.139536015 | 0.476 | 0.439 | 1.63E−33 |
| Cpox | 6.95E−38 | 0.109354164 | 0.628 | 0.45 | 1.73E−33 |
| Smlr1 | 9.30E−38 | −0.143751976 | 0.327 | 0.314 | 2.31E−33 |
| Mgea5 | 9.46E−38 | 0.115140568 | 0.436 | 0.268 | 2.35E−33 |
| Mrpl14 | 9.59E−38 | −0.133065855 | 0.567 | 0.523 | 2.38E−33 |
| 150001 1K16Rik | 9.85E−38 | −0.156897838 | 0.297 | 0.312 | 2.45E−33 |
| Lonp1 | 1.03E−37 | 0.11924964 | 0.65 | 0.468 | 2.56E−33 |
| Ube2h | 1.08E−37 | −0.109697361 | 0.257 | 0.228 | 2.69E−33 |
| Smg1 | 1.16E−37 | 0.102024606 | 0.24 | 0.106 | 2.89E−33 |
| Slc37a4 | 1.47E−37 | 0.154508476 | 0.687 | 0.502 | 3.66E−33 |
| Hfe2 | 1.73E−37 | −0.100511022 | 0.332 | 0.301 | 4.29E−33 |
| Dgcr6 | 2.21E−37 | −0.112390889 | 0.423 | 0.373 | 5.50E−33 |
| Sfxn1 | 3.26E−37 | 0.177292254 | 0.68 | 0.496 | 8.10E−33 |
| Churc1 | 4.30E−37 | −0.112101868 | 0.199 | 0.186 | 1.07E−32 |
| Mrpl27 | 4.60E−37 | −0.130784318 | 0.287 | 0.275 | 1.14E−32 |
| Hadha | 4.81E−37 | 0.125402769 | 0.888 | 0.744 | 1.19E−32 |
| Ubxn1 | 5.35E−37 | −0.107380689 | 0.655 | 0.57 | 1.33E−32 |
| Abca8a | 6.50E−37 | 0.112860383 | 0.442 | 0.277 | 1.61E−32 |
| Entpd5 | 6.69E−37 | −0.114063163 | 0.647 | 0.578 | 1.66E−32 |
| 1110065P20Rik | 6.85E−37 | −0.110034234 | 0.3 | 0.273 | 1.70E−32 |
| Uqcrh | 7.79E−37 | −0.146211767 | 0.861 | 0.81 | 1.93E−32 |
| Emg1 | 9.41E−37 | −0.106005411 | 0.369 | 0.325 | 2.34E−32 |
| Dld | 9.96E−37 | 0.10036701 | 0.627 | 0.451 | 2.47E−32 |
| Cml2 | 1.19E−36 | 0.14309935 | 0.957 | 0.857 | 2.96E−32 |
| Tmem176a | 1.24E−36 | 0.129581291 | 0.612 | 0.429 | 3.08E−32 |
| Acox2 | 1.32E−36 | 0.125739523 | 0.696 | 0.517 | 3.28E−32 |
| Hmgcr | 1.98E−36 | 0.106466986 | 0.435 | 0.272 | 4.91E−32 |
| Mrps24 | 2.00E−36 | −0.139387979 | 0.608 | 0.566 | 4.96E−32 |
| Slc25a13 | 2.05E−36 | 0.118116385 | 0.819 | 0.655 | 5.09E−32 |
| Rpl18 | 2.11E−36 | −0.114603362 | 0.313 | 0.266 | 5.24E−32 |
| Smagp | 2.11E−36 | −0.108071023 | 0.23 | 0.209 | 5.25E−32 |
| Psmb5 | 2.24E−36 | −0.125235705 | 0.395 | 0.348 | 5.55E−32 |
| Serpinf2 | 2.74E−36 | 0.121084682 | 0.953 | 0.843 | 6.81E−32 |
| Slc25a23 | 2.79E−36 | 0.113100224 | 0.572 | 0.399 | 6.92E−32 |
| Eif3c | 3.08E−36 | 0.102123395 | 0.715 | 0.541 | 7.64E−32 |
| Rplp0 | 3.22E−36 | −0.152097782 | 0.828 | 0.791 | 8.00E−32 |
| Rnf181 | 3.86E−36 | −0.118990143 | 0.35 | 0.318 | 9.59E−32 |
| Dnajc3 | 3.88E−36 | 0.122737182 | 0.906 | 0.772 | 9.64E−32 |
| Sdf2 | 4.05E−36 | −0.108593657 | 0.301 | 0.279 | 1.00E−31 |
| Tsc22d3 | 4.47E−36 | −0.117456945 | 0.326 | 0.312 | 1.11E−31 |
| Rplp1 | 4.72E−36 | −0.196523046 | 0.782 | 0.757 | 1.17E−31 |
| Ifi27 | 4.81E−36 | 0.228184801 | 0.632 | 0.453 | 1.19E−31 |
| Mrpl18 | 5.29E−36 | −0.106603574 | 0.391 | 0.343 | 1.31E−31 |
| Ube2b | 5.62E−36 | 0.140018225 | 0.657 | 0.478 | 1.39E−31 |
| Gpx4 | 6.41E−36 | −0.101573086 | 0.301 | 0.25 | 1.59E−31 |
| Mrpl33 | 7.02E−36 | −0.105537953 | 0.471 | 0.419 | 1.74E−31 |
| Ssr4 | 7.96E−36 | −0.131894778 | 0.736 | 0.687 | 1.98E−31 |
| Naprt1 | 8.70E−36 | −0.112711561 | 0.56 | 0.492 | 2.16E−31 |
| Hsp90ab1 | 9.49E−36 | 0.137555293 | 0.982 | 0.922 | 2.36E−31 |
| Hsd17b12 | 1.08E−35 | 0.150192807 | 0.811 | 0.647 | 2.69E−31 |
| Smdt1 | 1.45E−35 | −0.136251907 | 0.623 | 0.573 | 3.59E−31 |
| 1100001G20Rik | 1.79E−35 | −0.181522579 | 0.998 | 0.982 | 4.44E−31 |
| Pxdc1 | 1.82E−35 | −0.133589092 | 0.345 | 0.333 | 4.53E−31 |
| Gm11273 | 2.05E−35 | −0.154535944 | 0.034 | 0.102 | 5.09E−31 |
| 1110001J03Rik | 3.16E−35 | −0.131166164 | 0.498 | 0.456 | 7.84E−31 |
| Cald1 | 3.35E−35 | 0.130849221 | 0.889 | 0.749 | 8.32E−31 |
| Lsmd1 | 3.46E−35 | −0.107665711 | 0.404 | 0.357 | 8.59E−31 |
| Snord118 | 3.53E−35 | 0.102458085 | 0.401 | 0.248 | 8.78E−31 |
| Gnpnat1 | 3.96E−35 | −0.102162123 | 0.309 | 0.279 | 9.84E−31 |
| H2afv | 5.34E−35 | −0.108111525 | 0.291 | 0.275 | 1.33E−30 |
| Hspa8 | 5.38E−35 | 0.148342283 | 0.889 | 0.756 | 1.34E−30 |
| Stard5 | 6.66E−35 | −0.106226141 | 0.351 | 0.315 | 1.65E−30 |
| Cyp2d9 | 6.68E−35 | −0.10468367 | 0.997 | 0.959 | 1.66E−30 |
| Atf4 | 1.03E−34 | 0.170664039 | 0.622 | 0.442 | 2.56E−30 |
| Cluh | 1.34E−34 | 0.10709751 | 0.801 | 0.641 | 3.32E−30 |
| Tmem56 | 1.51E−34 | 0.130642059 | 0.779 | 0.612 | 3.75E−30 |
| Rps9 | 1.57E−34 | −0.160672445 | 0.733 | 0.675 | 3.89E−30 |
| Rnf213 | 2.52E−34 | 0.120260244 | 0.258 | 0.123 | 6.25E−30 |
| Fam162a | 2.59E−34 | −0.115030119 | 0.595 | 0.53 | 6.43E−30 |
| Oplah | 2.74E−34 | 0.10196367 | 0.626 | 0.455 | 6.81E−30 |
| Dhrs4 | 3.20E−34 | −0.115542613 | 0.773 | 0.69 | 7.96E−30 |
| 2310001H17Rik | 3.22E−34 | −0.145935249 | 0.163 | 0.212 | 8.00E−30 |
| Anapc13 | 3.37E−34 | −0.103891949 | 0.501 | 0.427 | 8.38E−30 |
| 2810459M11Rik | 3.87E−34 | −0.128519254 | 0.303 | 0.298 | 9.60E−30 |
| Mrpl11 | 4.74E−34 | −0.11725047 | 0.376 | 0.345 | 1.18E−29 |
| Irf6 | 5.40E−34 | −0.108059035 | 0.212 | 0.214 | 1.34E−29 |
| Slc25a3 | 6.95E−34 | 0.149859347 | 0.89 | 0.758 | 1.73E−29 |
| Rpl13 | 8.40E−34 | −0.102068358 | 0.536 | 0.464 | 2.09E−29 |
| Rpl13a-ps1 | 9.43E−34 | −0.136090818 | 0.03 | 0.102 | 2.34E−29 |
| Pnpla8 | 1.02E−33 | 0.125201153 | 0.715 | 0.544 | 2.54E−29 |
| Camk1d | 1.06E−33 | 0.130256351 | 0.405 | 0.247 | 2.64E−29 |
| Pa2g4 | 1.17E−33 | 0.112190082 | 0.589 | 0.418 | 2.90E−29 |
| Txn1 | 1.37E−33 | −0.116589612 | 0.869 | 0.809 | 3.41E−29 |
| Serf2 | 1.38E−33 | −0.102114553 | 0.271 | 0.237 | 3.43E−29 |
| Arrdc3 | 1.74E−33 | −0.1460998 | 0.208 | 0.213 | 4.32E−29 |
| Ephx1 | 1.80E−33 | −0.102641923 | 0.874 | 0.8 | 4.48E−29 |
| Sgpl1 | 1.84E−33 | −0.102012642 | 0.344 | 0.3 | 4.57E−29 |
| mt-Ti | 1.95E−33 | −0.175221151 | 0.091 | 0.167 | 4.85E−29 |
| Chac2 | 2.43E−33 | −0.116526774 | 0.25 | 0.251 | 6.03E−29 |
| Gm2000 | 2.73E−33 | −0.101274241 | 0.168 | 0.137 | 6.77E−29 |
| Rnf11 | 2.96E−33 | 0.115434821 | 0.646 | 0.476 | 7.36E−29 |
| Etnppl | 3.39E−33 | 0.199542145 | 0.769 | 0.606 | 8.41E−29 |
| Sdr42e1 | 3.62E−33 | −0.10244655 | 0.411 | 0.363 | 8.99E−29 |
| Fahd1 | 3.64E−33 | −0.119584582 | 0.287 | 0.272 | 9.03E−29 |
| Krt18 | 3.68E−33 | 0.185376047 | 0.771 | 0.617 | 9.15E−29 |
| Cstf3 | 5.02E−33 | −0.130987349 | 0.128 | 0.186 | 1.25E−28 |
| Alkbh7 | 5.19E−33 | −0.126292957 | 0.202 | 0.215 | 1.29E−28 |
| Srp14 | 5.79E−33 | −0.103296322 | 0.396 | 0.359 | 1.44E−28 |
| Etfdh | 1.05E−32 | 0.125403001 | 0.847 | 0.7 | 2.61E−28 |
| Acaa2 | 1.17E−32 | 0.112138898 | 0.996 | 0.96 | 2.91E−28 |
| Mxd4 | 1.19E−32 | −0.100169237 | 0.358 | 0.321 | 2.96E−28 |
| F9 | 1.37E−32 | 0.120261398 | 0.791 | 0.632 | 3.41E−28 |
| H2-Q10 | 1.94E−32 | 0.109441992 | 1 | 0.982 | 4.83E−28 |
| Mup20 | 2.37E−32 | −0.244695365 | 1 | 1 | 5.87E−28 |
| Mcfd2 | 2.42E−32 | 0.109751379 | 0.798 | 0.643 | 6.00E−28 |
| Ugt3a2 | 2.43E−32 | 0.110842492 | 0.836 | 0.689 | 6.04E−28 |
| Slc19a2 | 2.66E−32 | −0.105653788 | 0.315 | 0.293 | 6.61E−28 |
| Cox5b | 2.83E−32 | −0.184963493 | 0.747 | 0.726 | 7.02E−28 |
| C6 | 4.05E−32 | 0.126254945 | 0.798 | 0.642 | 1.01E−27 |
| Psmb4 | 5.34E−32 | −0.1235222 | 0.764 | 0.705 | 1.33E−27 |
| Gm2788 | 5.45E−32 | −0.119044189 | 0.279 | 0.264 | 1.35E−27 |
| Cyp2d10 | 5.96E−32 | −0.114162001 | 0.958 | 0.921 | 1.48E−27 |
| Bcar3 | 6.99E−32 | −0.154603512 | 0.132 | 0.193 | 1.74E−27 |
| Wdr89 | 7.20E−32 | −0.157854036 | 0.08 | 0.118 | 1.79E−27 |
| Polr2f | 1.08E−31 | −0.10168269 | 0.363 | 0.321 | 2.68E−27 |
| Gm16157 | 1.10E−31 | −0.100720355 | 0.215 | 0.197 | 2.73E−27 |
| Immp1l | 1.14E−31 | −0.117878413 | 0.317 | 0.313 | 2.83E−27 |
| Lect2 | 1.87E−31 | −0.274252442 | 0.297 | 0.414 | 4.64E−27 |
| Acsl1 | 2.77E−31 | −0.149382996 | 0.958 | 0.924 | 6.89E−27 |
| Maob | 3.15E−31 | 0.107182064 | 0.734 | 0.575 | 7.82E−27 |
| Gm6484 | 3.49E−31 | 0.123791929 | 0.314 | 0.174 | 8.67E−27 |
| Ybx1 | 4.79E−31 | −0.108005701 | 0.685 | 0.605 | 1.19E−26 |
| Pla2g12b | 6.31E−31 | −0.10068138 | 0.517 | 0.463 | 1.57E−26 |
| Pithd1 | 6.72E−31 | −0.101630538 | 0.177 | 0.184 | 1.67E−26 |
| 1810009A15Rik | 7.82E−31 | −0.105379118 | 0.227 | 0.218 | 1.94E−26 |
| Hnrnpa2b1 | 7.98E−31 | 0.119047009 | 0.86 | 0.722 | 1.98E−26 |
| Tecr | 9.10E−31 | 0.101458193 | 0.647 | 0.486 | 2.26E−26 |
| Ddt | 1.69E−30 | −0.172944365 | 0.847 | 0.829 | 4.20E−26 |
| Ccl9 | 1.84E−30 | −0.101196684 | 0.534 | 0.486 | 4.57E−26 |
| Gm13773 | 2.84E−30 | −0.13782302 | 0.047 | 0.11 | 7.05E−26 |
| Tubb5 | 3.08E−30 | −0.118258355 | 0.276 | 0.271 | 7.64E−26 |
| Gm11789 | 3.31E−30 | −0.107724995 | 0.235 | 0.228 | 8.22E−26 |
| Rps21 | 4.02E−30 | −0.113921701 | 0.651 | 0.579 | 9.98E−26 |
| Slc40a1 | 4.18E−30 | −0.111681574 | 0.353 | 0.328 | 1.04E−25 |
| Ide | 4.60E−30 | −0.128451427 | 0.524 | 0.494 | 1.14E−25 |
| Urah | 5.21E−30 | −0.109034054 | 0.911 | 0.852 | 1.29E−25 |
| 1500012F01Rik | 6.40E−30 | −0.116078802 | 0.2 | 0.205 | 1.59E−25 |
| Cml5 | 1.19E−29 | −0.124698142 | 0.151 | 0.178 | 2.94E−25 |
| Cenpv | 1.47E−29 | −0.10852708 | 0.222 | 0.21 | 3.64E−25 |
| Sh3bp5 | 1.70E−29 | −0.131584862 | 0.191 | 0.21 | 4.22E−25 |
| Lsr | 1.70E−29 | 0.118122924 | 0.677 | 0.517 | 4.22E−25 |
| Hsd17b10 | 1.71E−29 | −0.114042727 | 0.759 | 0.7 | 4.25E−25 |
| Acadvl | 1.87E−29 | 0.126009037 | 0.9 | 0.783 | 4.65E−25 |
| Creb3l3 | 1.90E−29 | 0.124229468 | 0.93 | 0.831 | 4.71E−25 |
| Gm10275 | 2.32E−29 | −0.131334292 | 0.068 | 0.126 | 5.76E−25 |
| Ptms | 2.41E−29 | 0.110348643 | 0.831 | 0.689 | 5.99E−25 |
| Cyp2c68 | 3.17E−29 | −0.113343173 | 0.593 | 0.558 | 7.87E−25 |
| Cyp4v3 | 5.18E−29 | 0.136434082 | 0.932 | 0.827 | 1.29E−24 |
| Sod2 | 6.44E−29 | 0.100502591 | 0.828 | 0.687 | 1.60E−24 |
| Hjurp | 7.95E−29 | −0.138156531 | 0.223 | 0.219 | 1.97E−24 |
| Saa3 | 8.86E−29 | 0.298054041 | 0.195 | 0.087 | 2.20E−24 |
| C8b | 9.77E−29 | 0.113345886 | 0.963 | 0.881 | 2.43E−24 |
| Ctsz | 1.05E−28 | 0.125462706 | 0.789 | 0.641 | 2.60E−24 |
| Hdhd3 | 1.06E−28 | −0.120077709 | 0.172 | 0.201 | 2.63E−24 |
| Rpl31 | 2.64E−28 | −0.114404945 | 0.166 | 0.16 | 6.54E−24 |
| Paqr7 | 3.16E−28 | −0.115007751 | 0.144 | 0.159 | 7.84E−24 |
| Bola3 | 5.01E−28 | −0.115283165 | 0.574 | 0.525 | 1.24E−23 |
| Oat | 5.60E−28 | −0.255537891 | 0.502 | 0.611 | 1.39E−23 |
| Serpina12 | 6.30E−28 | 0.135573206 | 0.648 | 0.492 | 1.56E−23 |
| Csad | 9.84E−28 | 0.204004276 | 0.872 | 0.744 | 2.44E−23 |
| Slc46a3 | 1.39E−27 | −0.111772252 | 0.203 | 0.211 | 3.44E−23 |
| Sdhaf1 | 5.54E−27 | −0.10322437 | 0.113 | 0.138 | 1.37E−22 |
| Rgn | 6.33E−27 | −0.13339713 | 0.807 | 0.874 | 1.57E−22 |
| Tyw5 | 1.29E−26 | −0.115955707 | 0.141 | 0.161 | 3.20E−22 |
| Bok | 1.52E−26 | −0.107596384 | 0.062 | 0.111 | 3.78E−22 |
| Nr2f2 | 3.29E−26 | −0.135717133 | 0.11 | 0.163 | 8.16E−22 |
| Clec2h | 1.15E−25 | −0.120494736 | 0.059 | 0.126 | 2.85E−21 |
| Ebp | 2.12E−25 | 0.120141088 | 0.801 | 0.666 | 5.25E−21 |
| Aldh2 | 2.19E−25 | 0.108487481 | 0.994 | 0.973 | 5.44E−21 |
| Ctsc | 8.47E−25 | 0.12557031 | 0.413 | 0.271 | 2.10E−20 |
| Apoh | 1.17E−24 | −0.113138266 | 0.997 | 0.986 | 2.91E−20 |
| mmu-mir-6240 | 3.74E−24 | −0.197588471 | 0.054 | 0.115 | 9.29E−20 |
| Mrap | 5.13E−24 | 0.100266553 | 0.408 | 0.272 | 1.27E−19 |
| 9030616G12Rik | 8.16E−23 | −0.113944704 | 0.075 | 0.126 | 2.03E−18 |
| Serpina1a | 1.69E−22 | −0.130631577 | 0.998 | 0.991 | 4.20E−18 |
| Car3 | 2.72E−22 | −0.118093283 | 0.999 | 0.974 | 6.75E−18 |
| Cox5a | 4.36E−22 | 0.119858632 | 0.822 | 0.702 | 1.08E−17 |
| Pigr | 6.75E−22 | 0.116709563 | 0.988 | 0.947 | 1.68E−17 |
| Dlc1 | 2.09E−21 | −0.12318528 | 0.1 | 0.148 | 5.18E−17 |
| Cyp7a1 | 2.57E−21 | −0.191761184 | 0.186 | 0.259 | 6.39E−17 |
| Gulo | 1.21E−20 | −0.161523894 | 0.482 | 0.497 | 2.99E−16 |
| Cyp26a1 | 2.63E−18 | −0.115338737 | 0.155 | 0.184 | 6.52E−14 |
| Acaa1a | 7.70E−18 | −0.106217042 | 0.735 | 0.679 | 1.91E−13 |
| Fitm1 | 5.83E−16 | −0.136552777 | 0.175 | 0.208 | 1.45E−11 |
| Fam13a | 3.40E−14 | −0.101488514 | 0.102 | 0.118 | 8.43E−10 |
| Cyp4a14 | 1.89E−13 | 0.136365312 | 0.541 | 0.431 | 4.70E−09 |
| Gnmt | 1.77E−10 | −0.105899231 | 0.999 | 0.988 | 4.39E−06 |
| Trf | 6.06E−10 | −0.106899897 | 0.999 | 1 | 1.51E−05 |
| Aldh3a2 | 6.02E−09 | −0.141606596 | 0.454 | 0.529 | 0.000149421 |
| Mup7 | 0.007918627 | −0.293978105 | 0.998 | 0.998 | 1 |
| Hamp | 0.31057435 | 0.10479673 | 0.888 | 0.884 | 1 |

TABLE 5b
Differentially expressed genes between time point PHX3 FC.2 and untreated (UT).

| p_val | avg_logFC | pct.1 | pct.2 | p_val_adj | |
|---|---|---|---|---|---|
| Saa2 | 0 | 2.971862499 | 0.999 | 0.222 | 0 |
| Mt1 | 0 | 2.610854994 | 1 | 0.556 | 0 |
| Mt2 | 0 | 2.6060431 | 1 | 0.365 | 0 |
| Saa1 | 0 | 2.202156726 | 1 | 0.662 | 0 |
| Steap4 | 0 | 1.890015167 | 0.973 | 0.222 | 0 |
| Serpina3n | 0 | 1.759758939 | 0.992 | 0.574 | 0 |
| Lcn2 | 0 | 1.499141183 | 0.815 | 0.07 | 0 |
| Lpin1 | 0 | 1.327657625 | 0.94 | 0.31 | 0 |
| n-R5-8s1 | 0 | 1.289598504 | 0.805 | 0.031 | 0 |
| Igfbp1 | 0 | 1.288789902 | 0.99 | 0.717 | 0 |
| Cxcl1 | 0 | 1.230398019 | 0.919 | 0.314 | 0 |
| Scd1 | 0 | 1.081676999 | 0.998 | 0.901 | 0 |
| Gm26924 | 0 | 1.063463323 | 1 | 1 | 0 |
| Il1r1 | 0 | 0.998168131 | 0.8 | 0.167 | 0 |
| Fga | 0 | 0.981249724 | 1 | 0.997 | 0 |
| Gm24601 | 0 | 0.924568273 | 0.661 | 0.015 | 0 |
| Nnmt | 0 | 0.901052904 | 0.97 | 0.716 | 0 |
| Itih4 | 0 | 0.887186241 | 0.999 | 0.948 | 0 |
| St3gal5 | 0 | 0.882761517 | 0.915 | 0.49 | 0 |
| Tacc2 | 0 | 0.877623973 | 0.707 | 0.071 | 0 |
| Lrg1 | 0 | 0.870250525 | 0.981 | 0.736 | 0 |
| Fam134b | 0 | 0.865578177 | 0.876 | 0.405 | 0 |
| Tat | 0 | 0.857965136 | 0.998 | 0.903 | 0 |
| Saa4 | 0 | 0.854921603 | 0.981 | 0.749 | 0 |
| Thrsp | 0 | 0.836598219 | 0.98 | 0.627 | 0 |
| Slc39a14 | 0 | 0.831584936 | 0.849 | 0.343 | 0 |
| Lars2 | 0 | 0.816795688 | 1 | 0.994 | 0 |
| Fgg | 0 | 0.809322713 | 1 | 0.991 | 0 |
| Slc38a2 | 0 | 0.797899442 | 0.874 | 0.413 | 0 |
| Serpina3m | 0 | 0.779052745 | 0.961 | 0.722 | 0 |
| Hp | 0 | 0.774785279 | 1 | 0.999 | 0 |
| Fgb | 0 | 0.739519125 | 1 | 1 | 0 |
| Gm15564 | 0 | 0.735157137 | 0.999 | 0.956 | 0 |
| Serpina10 | 0 | 0.733655099 | 0.934 | 0.641 | 0 |
| Plin2 | 0 | 0.720463076 | 0.952 | 0.714 | 0 |
| Aass | 0 | 0.705615345 | 0.965 | 0.716 | 0 |
| Fmo5 | 0 | 0.690160801 | 0.991 | 0.837 | 0 |
| Fn1 | 0 | 0.684266092 | 0.983 | 0.821 | 0 |
| Fkbp5 | 0 | 0.663304836 | 0.665 | 0.117 | 0 |
| Slc25a47 | 0 | 0.635928777 | 0.994 | 0.927 | 0 |
| Mat1a | 0 | 0.623734998 | 0.999 | 0.966 | 0 |
| Aldob | 0 | 0.490200848 | 1 | 0.996 | 0 |
| Mup3 | 0 | −0.526410044 | 1 | 1 | 0 |
| Cyb5 | 0 | −0.71965743 | 0.992 | 0.983 | 0 |
| mt-Rnr1 | 0 | −0.872186844 | 1 | 1 | 0 |
| mt-Rnr2 | 0 | −0.873557854 | 1 | 1 | 0 |
| Mup11 | 0 | −0.976755523 | 0.996 | 1 | 0 |
| mt-Nd2 | 0 | −1.128776891 | 0.996 | 0.999 | 0 |
| mt-Nd1 | 0 | −1.136736863 | 0.999 | 1 | 0 |
| mt-Nd5 | 0 | −1.145550707 | 0.974 | 0.995 | 0 |
| mt-Co3 | 0 | −1.233961828 | 0.106 | 0.603 | 0 |
| Gm13775 | 0 | −1.255657303 | 0.205 | 0.761 | 0 |
| mt-Cytb | 0 | −1.296043506 | 0.993 | 0.999 | 0 |
| mt-Co2 | 0 | −1.341686736 | 0.1 | 0.629 | 0 |
| mt-Co1 | 0 | −1.381307687 | 0.986 | 0.999 | 0 |
| mt-Nd4 | 0 | −1.630375247 | 0.977 | 0.998 | 0 |
| Mup4 | 0 | −1.805301681 | 0.374 | 0.898 | 0 |
| Mup6 | 0 | −2.386597202 | 0.714 | 0.992 | 0 |
| Mup9 | 0 | −2.453596744 | 0.401 | 0.976 | 0 |
| Mup17 | 0 | −2.514472885 | 0.899 | 1 | 0 |
| Mup18 | 0 | −2.617739049 | 0.547 | 0.997 | 0 |
| Mup19 | 0 | −2.932276479 | 0.84 | 1 | 0 |
| Acox1 | 1.48219693752374e−323 | 0.540022158 | 0.998 | 0.966 | 3.68059143525895e−319 |
| Hnf4a | 5.92878775009496e−323 | 0.644195253 | 0.969 | 0.743 | 1.47223657410358e−318 |
| B3galt1 | 1.64029794419294e−321 | 0.788734938 | 0.634 | 0.163 | 4.0731878550199e−317 |
| Cyp2b10 | 2.20501497738948e−320 | −1.254972098 | 0.132 | 0.542 | 5.47549319185356e−316 |
| Mup5 | 4.03503412958546e−319 | −1.484560547 | 0.52 | 0.839 | 1.00197967505866e−314 |
| H3f3b | 7.69670285060869e−319 | 0.755567107 | 0.831 | 0.419 | 1.91124525186315e−314 |
| Btg2 | 6.7929354703014e−310 | 0.772686832 | 0.708 | 0.241 | 1.69E−305 |
| Sephs2 | 7.26878065222103e−310 | 0.618707003 | 0.974 | 0.761 | 1.80E−305 |
| Ankrd55 | 1.43E−296 | −1.464696038 | 0.374 | 0.696 | 3.54E−292 |
| Hspb8 | 1.52E−295 | 0.667847253 | 0.914 | 0.597 | 3.78E−291 |
| Slc7a2 | 4.69E−288 | 0.622326529 | 0.964 | 0.735 | 1.17E−283 |
| Cldn14 | 8.97E−284 | 0.511583526 | 0.505 | 0.076 | 2.23E−279 |
| C3 | 4.33E−282 | 0.482674202 | 0.999 | 0.99 | 1.07E−277 |
| Gjb2 | 5.86E−278 | 0.639507436 | 0.901 | 0.572 | 1.46E−273 |
| Rcan1 | 1.11E−277 | 0.509239866 | 0.522 | 0.085 | 2.75E−273 |
| Errfi1 | 7.45E−268 | 0.500850526 | 0.998 | 0.911 | 1.85E−263 |
| Pfkfb3 | 1.00E−265 | 0.426580733 | 0.421 | 0.039 | 2.50E−261 |
| Arhgef26 | 1.50E−264 | 0.568544702 | 0.67 | 0.203 | 3.71E−260 |
| Cyp2c29 | 3.44E−262 | −1.027267705 | 0.751 | 0.93 | 8.53E−258 |
| Egfr | 8.47E−255 | 0.555834247 | 0.989 | 0.809 | 2.10E−250 |
| Gsta3 | 1.25E−250 | −0.53784222 | 0.993 | 0.956 | 3.11E−246 |
| Bcl3 | 1.67E−248 | 0.419650143 | 0.479 | 0.074 | 4.14E−244 |
| Snx10 | 3.08E−247 | 0.558244633 | 0.63 | 0.188 | 7.65E−243 |
| Pck1 | 7.20E−247 | 0.43447474 | 0.999 | 0.987 | 1.79E−242 |
| Mup16 | 3.02E−246 | −0.848726713 | 0.816 | 0.934 | 7.49E−242 |
| Bach1 | 1.01E−243 | 0.533237374 | 0.589 | 0.151 | 2.51E−239 |
| Tifa | 8.52E−243 | 0.392339477 | 0.393 | 0.036 | 2.12E−238 |
| Hpx | 7.87E−242 | 0.460272423 | 1 | 0.997 | 1.95E−237 |
| Ugt2b5 | 6.48E−241 | −0.423085187 | 0.991 | 0.959 | 1.61E−236 |
| Etnk2 | 2.21E−240 | 0.592353201 | 0.918 | 0.655 | 5.48E−236 |
| 2010003K11Rik | 8.37E−239 | 0.372219971 | 0.412 | 0.046 | 2.08E−234 |
| Rapgef4 | 8.03E−238 | 0.588317735 | 0.836 | 0.436 | 1.99E−233 |
| Tiparp | 5.00E−233 | 0.489122153 | 0.485 | 0.087 | 1.24E−228 |
| Gpt2 | 2.89E−230 | 0.533331785 | 0.949 | 0.725 | 7.18E−226 |
| Slc25a25 | 6.62E−227 | 0.544465891 | 0.945 | 0.704 | 1.64E−222 |
| Mup10 | 6.77E−224 | 0.428268029 | 0.965 | 0.692 | 1.68E−219 |
| Txnip | 3.37E−223 | 0.552612237 | 0.741 | 0.297 | 8.37E−219 |
| Cyp2c50 | 9.25E−220 | −0.763912176 | 0.693 | 0.888 | 2.30E−215 |
| Xbp1 | 2.56E−219 | 0.539986 | 0.89 | 0.57 | 6.35E−215 |
| Upp2 | 4.48E−218 | 0.541753512 | 0.934 | 0.673 | 1.11E−213 |
| Mfsd2a | 1.49E−217 | 0.496351884 | 0.563 | 0.148 | 3.69E−213 |
| Actb | 1.79E−216 | 0.456981443 | 0.99 | 0.886 | 4.44E−212 |
| mmu-mir-6236 | 6.91E−215 | 0.468866739 | 0.989 | 0.837 | 1.72E−210 |
| Fbf1 | 1.36E−212 | 0.298573909 | 0.336 | 0.027 | 3.38E−208 |
| Hsd3b5 | 1.76E−209 | −0.587528886 | 0.49 | 0.587 | 4.36E−205 |
| Trib1 | 2.60E−209 | 0.581754281 | 0.705 | 0.308 | 6.45E−205 |
| Arg1 | 4.87E−205 | 0.381616351 | 1 | 0.982 | 1.21E−200 |
| Plin5 | 2.75E−204 | 0.489257529 | 0.736 | 0.315 | 6.82E−200 |
| Plk3 | 4.61E−204 | 0.488016367 | 0.644 | 0.226 | 1.15E−199 |
| Mup13 | 1.15E−203 | −0.755608269 | 0.303 | 0.546 | 2.86E−199 |
| Mtss1 | 5.64E−203 | 0.552137981 | 0.812 | 0.454 | 1.40E−198 |
| Klf6 | 1.08E−202 | 0.3616937 | 0.346 | 0.034 | 2.69E−198 |
| Itih3 | 6.73E−202 | 0.469631651 | 0.988 | 0.89 | 1.67E−197 |
| Gm23935 | 3.21E−201 | 0.498066925 | 0.999 | 0.925 | 7.96E−197 |
| Mup21 | 2.39E−199 | −0.668770594 | 0.829 | 0.929 | 5.93E−195 |
| Adck3 | 5.76E−199 | −0.482940158 | 0.729 | 0.764 | 1.43E−194 |
| Cyp2c54 | 3.82E−198 | −0.712859584 | 0.491 | 0.759 | 9.48E−194 |
| Serpina3c | 1.71E−196 | −0.554657351 | 0.118 | 0.385 | 4.23E−192 |
| Slc10a2 | 2.81E−196 | 0.30563824 | 0.376 | 0.05 | 6.99E−192 |
| Sdc4 | 3.39E−196 | 0.43560504 | 0.986 | 0.867 | 8.41E−192 |
| Egr1 | 3.50E−196 | 0.620480469 | 0.79 | 0.447 | 8.70E−192 |
| Apcs | 4.35E−195 | 0.573439593 | 0.854 | 0.525 | 1.08E−190 |
| Atf3 | 8.39E−194 | 0.597274889 | 0.56 | 0.179 | 2.08E−189 |
| Hsd3b7 | 1.35E−191 | −0.451728474 | 0.795 | 0.798 | 3.36E−187 |
| Orm1 | 2.91E−190 | 0.463411533 | 0.993 | 0.952 | 7.24E−186 |
| Pzp | 6.72E−190 | 0.387351113 | 0.999 | 0.986 | 1.67E−185 |
| Glul | 9.26E−190 | 0.478908893 | 0.58 | 0.18 | 2.30E−185 |
| Prg4 | 6.84E−189 | 0.480504574 | 0.585 | 0.188 | 1.70E−184 |
| Fgl1 | 1.44E−188 | 0.49338252 | 0.961 | 0.784 | 3.57E−184 |
| Gpcpd1 | 3.50E−188 | 0.53780646 | 0.778 | 0.398 | 8.70E−184 |
| Klf10 | 4.90E−188 | 0.304779554 | 0.388 | 0.06 | 1.22E−183 |
| Rgs16 | 2.29E−187 | 0.438105488 | 0.482 | 0.113 | 5.68E−183 |
| Ptp4a1 | 3.23E−187 | 0.393137367 | 0.528 | 0.145 | 8.02E−183 |
| mt-Atp6 | 3.00E−186 | −0.852555757 | 0.138 | 0.452 | 7.45E−182 |
| Fam35a | 2.25E−184 | 0.470461753 | 0.642 | 0.246 | 5.59E−180 |
| Rhob | 2.93E−184 | 0.37207281 | 0.4 | 0.069 | 7.28E−180 |
| Mup15 | 6.78E−184 | −1.074409915 | 0.357 | 0.617 | 1.68E−179 |
| Cyp3a25 | 7.66E−184 | −0.41051207 | 0.928 | 0.919 | 1.90E−179 |
| Got1 | 1.96E−183 | 0.542891511 | 0.866 | 0.587 | 4.86E−179 |
| Ces1d | 6.99E−180 | −0.385155368 | 0.944 | 0.923 | 1.74E−175 |
| Shb | 1.09E−178 | 0.430613558 | 0.589 | 0.204 | 2.71E−174 |
| Sds | 5.06E−177 | 0.787094904 | 0.868 | 0.67 | 1.26E−172 |
| Abca1 | 3.96E−176 | 0.50506234 | 0.756 | 0.377 | 9.82E−172 |
| 1810055G02Rik | 7.47E−174 | 0.382584093 | 0.528 | 0.155 | 1.86E−169 |
| Stat3 | 9.40E−173 | 0.460515229 | 0.701 | 0.318 | 2.33E−168 |
| Akr1c6 | 3.12E−171 | −0.43492211 | 0.992 | 0.971 | 7.75E−167 |
| Apoe | 6.43E−171 | −0.26209828 | 1 | 1 | 1.60E−166 |
| Igfbp4 | 4.07E−170 | 0.387861679 | 0.986 | 0.866 | 1.01E−165 |
| Cdkn1a | 2.71E−169 | 0.449325471 | 0.572 | 0.195 | 6.74E−165 |
| Gfra1 | 5.61E−169 | 0.474694426 | 0.835 | 0.498 | 1.39E−164 |
| Gc | 9.68E−169 | 0.233764264 | 1 | 0.999 | 2.40E−164 |
| Nrg4 | 3.42E−168 | 0.246089001 | 0.306 | 0.034 | 8.49E−164 |
| Ivns1abp | 1.34E−166 | 0.46254094 | 0.688 | 0.315 | 3.32E−162 |
| Fh1 | 2.21E−165 | 0.471428771 | 0.915 | 0.648 | 5.48E−161 |
| Myo1e | 9.01E−164 | 0.399456672 | 0.551 | 0.182 | 2.24E−159 |
| Slc41a2 | 1.15E−163 | 0.3685439 | 0.482 | 0.134 | 2.86E−159 |
| Mup8 | 1.92E−163 | −0.793731191 | 0.111 | 0.373 | 4.78E−159 |
| Slc25a51 | 3.30E−163 | 0.372157664 | 0.522 | 0.161 | 8.21E−159 |
| Psen2 | 1.62E−162 | −0.419555606 | 0.749 | 0.774 | 4.03E−158 |
| Dak | 3.36E−162 | 0.481947664 | 0.867 | 0.563 | 8.34E−158 |
| Apoc4 | 3.03E−160 | −0.356086407 | 0.999 | 0.98 | 7.52E−156 |
| Fasn | 1.83E−159 | 0.467168076 | 0.719 | 0.345 | 4.54E−155 |
| Bhlhe40 | 9.28E−159 | 0.403249298 | 0.628 | 0.251 | 2.30E−154 |
| Tmem87b | 4.54E−156 | 0.288330587 | 0.415 | 0.096 | 1.13E−151 |
| Ddx3x | 1.61E−155 | 0.47100162 | 0.823 | 0.513 | 4.00E−151 |
| Ttr | 1.71E−155 | 0.248320061 | 1 | 1 | 4.24E−151 |
| Aldh1a1 | 6.92E−152 | −0.40245097 | 0.984 | 0.956 | 1.72E−147 |
| Slc25a22 | 2.16E−151 | 0.390931532 | 0.649 | 0.282 | 5.36E−147 |
| Rnf103 | 8.20E−150 | 0.438540394 | 0.701 | 0.35 | 2.04E−145 |
| Scara5 | 1.55E−149 | 0.221632911 | 0.324 | 0.054 | 3.85E−145 |
| B4galt1 | 3.66E−147 | 0.321380868 | 0.494 | 0.155 | 9.09E−143 |
| Rnf125 | 1.05E−146 | −0.463320018 | 0.312 | 0.493 | 2.62E−142 |
| Hacl1 | 1.08E−146 | −0.332899987 | 0.842 | 0.797 | 2.67E−142 |
| Gm26917 | 1.95E−145 | 0.46739621 | 0.523 | 0.181 | 4.85E−141 |
| Junb | 2.38E−145 | 0.458500682 | 0.788 | 0.47 | 5.91E−141 |
| Cps1 | 3.74E−145 | 0.336429448 | 0.999 | 0.971 | 9.30E−141 |
| Atp1b1 | 1.85E−144 | 0.424801231 | 0.567 | 0.226 | 4.59E−140 |
| Glud1 | 6.49E−144 | 0.341724783 | 0.991 | 0.905 | 1.61E−139 |
| D10Wsu102e | 2.64E−142 | 0.256022156 | 0.338 | 0.063 | 6.54E−138 |
| Hyou1 | 2.91E−142 | 0.431109873 | 0.731 | 0.385 | 7.23E−138 |
| Jmjd1c | 3.12E−142 | 0.39612601 | 0.516 | 0.178 | 7.75E−138 |
| Apoa5 | 6.44E−142 | 0.30259839 | 0.999 | 0.974 | 1.60E−137 |
| 4933426M11Rik | 8.78E−142 | 0.28624304 | 0.419 | 0.109 | 2.18E−137 |
| Scp2 | 3.84E−141 | 0.279911381 | 0.997 | 0.986 | 9.52E−137 |
| Jun | 2.06E−140 | 0.479246114 | 0.864 | 0.593 | 5.12E−136 |
| Mup12 | 3.48E−140 | −0.589618573 | 0.792 | 0.86 | 8.63E−136 |
| Cyp2a5 | 3.99E−140 | −0.938183249 | 0.278 | 0.555 | 9.91E−136 |
| Cpt1a | 7.36E−140 | 0.42860734 | 0.894 | 0.651 | 1.83E−135 |
| Pik3r1 | 1.27E−139 | 0.389193614 | 0.577 | 0.231 | 3.16E−135 |
| Apob | 4.15E−139 | 0.416824707 | 0.997 | 0.989 | 1.03E−134 |
| Epas1 | 1.69E−136 | 0.38425941 | 0.646 | 0.297 | 4.19E−132 |
| Ugt2b36 | 6.78E−136 | −0.287193139 | 0.978 | 0.93 | 1.68E−131 |
| Mbd1 | 2.07E−135 | 0.348094524 | 0.507 | 0.176 | 5.14E−131 |
| Mup2 | 5.79E−135 | −0.650107006 | 0.161 | 0.392 | 1.44E−130 |
| Foxq1 | 5.09E−134 | 0.22551931 | 0.377 | 0.096 | 1.26E−129 |
| Gadd45g | 2.13E−133 | 0.392031882 | 0.899 | 0.639 | 5.28E−129 |
| Fos | 5.48E−133 | 0.524733327 | 0.785 | 0.51 | 1.36E−128 |
| Bcl2l1 | 6.25E−133 | 0.280315334 | 0.426 | 0.122 | 1.55E−128 |
| Ptpn1 | 8.78E−133 | 0.268263225 | 0.402 | 0.107 | 2.18E−128 |
| Slc25a15 | 1.87E−132 | 0.402049896 | 0.931 | 0.738 | 4.63E−128 |
| Tdo2 | 5.72E−132 | 0.25156007 | 0.999 | 0.991 | 1.42E−127 |
| Rnd3 | 2.06E−131 | 0.285147682 | 0.375 | 0.091 | 5.11E−127 |
| Adh1 | 2.50E−131 | −0.295980789 | 0.995 | 0.96 | 6.22E−127 |
| Gm5096 | 4.91E−131 | −0.399548106 | 0.364 | 0.468 | 1.22E−126 |
| Litaf | 5.25E−131 | 0.396007707 | 0.603 | 0.278 | 1.30E−126 |
| Wbp1l | 8.03E−130 | 0.409138156 | 0.867 | 0.606 | 1.99E−125 |
| Grn | 4.87E−129 | 0.403315761 | 0.726 | 0.396 | 1.21E−124 |
| Tns1 | 5.97E−129 | 0.33981795 | 0.524 | 0.196 | 1.48E−124 |
| Cyp2c37 | 6.92E−129 | −0.662335734 | 0.314 | 0.608 | 1.72E−124 |
| Ahcy | 1.09E−128 | −0.375627724 | 0.918 | 0.916 | 2.71E−124 |
| Foxa3 | 4.50E−128 | 0.307992015 | 0.505 | 0.182 | 1.12E−123 |
| Qsox1 | 5.91E−128 | 0.378486857 | 0.925 | 0.707 | 1.47E−123 |
| Cth | 8.38E−128 | −0.308847665 | 0.898 | 0.838 | 2.08E−123 |
| Id2 | 2.52E−127 | 0.430147785 | 0.826 | 0.579 | 6.25E−123 |
| Myh9 | 8.87E−127 | 0.399128031 | 0.653 | 0.318 | 2.20E−122 |
| Cox6c | 5.03E−126 | −0.41128283 | 0.893 | 0.884 | 1.25E−121 |
| Raph1 | 2.97E−125 | 0.242670968 | 0.382 | 0.1 | 7.38E−121 |
| Ier2 | 4.45E−123 | 0.452598264 | 0.763 | 0.49 | 1.10E−118 |
| Cfh | 5.44E−123 | 0.312615767 | 0.991 | 0.897 | 1.35E−118 |
| Itih2 | 1.39E−122 | 0.336714211 | 0.984 | 0.884 | 3.45E−118 |
| Rsp14 | 1.25E−121 | −0.346340977 | 0.936 | 0.917 | 3.11E−117 |
| Cyp8b1 | 1.41E−121 | −0.364571013 | 0.714 | 0.745 | 3.50E−117 |
| Ccnl1 | 1.51E−121 | 0.341338448 | 0.526 | 0.206 | 3.75E−117 |
| Cox4i1 | 1.81E−121 | −0.288035158 | 0.992 | 0.958 | 4.50E−117 |
| Eef1a1 | 5.57E−121 | 0.340768186 | 0.967 | 0.85 | 1.38E−116 |
| Nedd4l | 2.65E−120 | 0.271827098 | 0.447 | 0.147 | 6.58E−116 |
| Inmt | 5.73E−120 | −0.308364158 | 0.966 | 0.884 | 1.42E−115 |
| Cep85l | 1.49E−119 | 0.202664771 | 0.347 | 0.089 | 3.71E−115 |
| Elovl5 | 6.23E−119 | 0.382267188 | 0.863 | 0.58 | 1.55E−114 |
| Zbtb16 | 6.92E−119 | 0.232600907 | 0.414 | 0.131 | 1.72E−114 |
| Il17ra | 2.85E−118 | 0.203529938 | 0.302 | 0.061 | 7.08E−114 |
| Hip1r | 3.33E−118 | 0.213858215 | 0.362 | 0.098 | 8.27E−114 |
| Plg | 4.13E−118 | 0.26619661 | 0.998 | 0.961 | 1.03E−113 |
| Eif4g2 | 6.98E−118 | 0.384302798 | 0.905 | 0.678 | 1.73E−113 |
| Atg14 | 1.11E−117 | 0.231648357 | 0.369 | 0.1 | 2.75E−113 |
| Ppip5k2 | 1.67E−117 | 0.30299148 | 0.51 | 0.197 | 4.16E−113 |
| Ulk1 | 3.53E−117 | 0.297841785 | 0.499 | 0.189 | 8.77E−113 |
| Pon1 | 1.04E−116 | −0.302819466 | 0.936 | 0.906 | 2.59E−112 |
| Elovl6 | 1.22E−116 | 0.37962641 | 0.553 | 0.24 | 3.04E−112 |
| Il6ra | 5.95E−116 | 0.251696836 | 0.431 | 0.142 | 1.48E−111 |
| Ndufb11 | 4.83E−115 | −0.36792318 | 0.722 | 0.735 | 1.20E−110 |
| Igfbp2 | 3.10E−113 | −0.348403589 | 0.886 | 0.885 | 7.70E−109 |
| Fads2 | 1.76E−112 | 0.323744791 | 0.592 | 0.273 | 4.37E−108 |
| Tnfrsf1a | 2.59E−112 | 0.346845029 | 0.551 | 0.244 | 6.43E−108 |
| Ugt2b34 | 3.49E−112 | −0.290735843 | 0.918 | 0.874 | 8.65E−108 |
| Gcnt2 | 3.49E−112 | 0.221618104 | 0.333 | 0.082 | 8.67E−108 |
| Ell2 | 5.30E−112 | 0.371745977 | 0.72 | 0.416 | 1.32E−107 |
| Ly6e | 5.39E−112 | 0.42022985 | 0.68 | 0.386 | 1.34E−107 |
| Rps2 | 1.20E−110 | −0.44250848 | 0.675 | 0.757 | 2.99E−106 |
| Epb4.1 | 3.29E−110 | 0.349430673 | 0.617 | 0.304 | 8.16E−106 |
| Ptpn2 | 1.67E−109 | 0.225408583 | 0.354 | 0.097 | 4.15E−105 |
| Clpx | 6.99E−109 | −0.306093278 | 0.818 | 0.783 | 1.74E−104 |
| Zc3h13 | 8.36E−109 | 0.204542393 | 0.325 | 0.083 | 2.08E−104 |
| Rps27l | 3.68E−108 | −0.361138678 | 0.855 | 0.849 | 9.13E−104 |
| Dst | 5.39E−108 | 0.329905553 | 0.507 | 0.206 | 1.34E−103 |
| Atl2 | 1.06E−107 | 0.321873809 | 0.674 | 0.361 | 2.64E−103 |
| Gm4076 | 1.66E−107 | −0.342157966 | 0.007 | 0.184 | 4.12E−103 |
| Gm20594 | 2.54E−107 | −0.559577825 | 0.108 | 0.294 | 6.30E−103 |
| Lpgat1 | 2.69E−107 | 0.390146943 | 0.776 | 0.502 | 6.68E−103 |
| Hmgcs1 | 5.90E−107 | −0.258009688 | 0.778 | 0.703 | 1.46E−102 |
| Esd | 1.57E−106 | −0.302906103 | 0.794 | 0.784 | 3.91E−102 |
| Oaz1 | 2.04E−106 | −0.339004868 | 0.759 | 0.75 | 5.06E−102 |
| Pptc7 | 7.95E−106 | 0.227095018 | 0.412 | 0.141 | 1.98E−101 |
| Sult1a1 | 1.09E−105 | 0.371023537 | 0.899 | 0.707 | 2.72E−101 |
| Nfil3 | 2.41E−105 | 0.338730712 | 0.617 | 0.309 | 5.98E−101 |
| Aqp8 | 3.51E−105 | −0.331182913 | 0.444 | 0.505 | 8.71E−101 |
| Gpx1 | 3.94E−105 | −0.222094695 | 1 | 0.999 | 9.79E−101 |
| Cyp3a11 | 4.68E−105 | −0.251219977 | 0.999 | 1 | 1.16E−100 |
| Cml1 | 2.22E−104 | −0.277339974 | 0.803 | 0.757 | 5.50E−100 |
| Apoa4 | 2.46E−104 | 0.609214916 | 0.604 | 0.339 | 6.11E−100 |
| Jak1 | 3.27E−104 | 0.362766687 | 0.648 | 0.342 | 8.12E−100 |
| Arid5b | 4.76E−104 | 0.232391653 | 0.472 | 0.186 | 1.18E−99 |
| Ralgapa2 | 2.31E−103 | 0.20791298 | 0.334 | 0.092 | 5.73E−99 |
| G6pc | 4.45E−103 | 0.341403933 | 0.948 | 0.752 | 1.11E−98 |
| Ifitm3 | 2.02E−102 | 0.331673739 | 0.958 | 0.812 | 5.02E−98 |
| Zfp36 | 2.26E−102 | 0.407014847 | 0.776 | 0.522 | 5.61E−98 |
| Oaf | 5.19E−102 | −0.333766768 | 0.79 | 0.794 | 1.29E−97 |
| Mgst1 | 5.80E−102 | −0.322742924 | 1 | 0.997 | 1.44E−97 |
| Uqcrq | 9.27E−102 | −0.316451873 | 0.98 | 0.951 | 2.30E−97 |
| Garem | 4.42E−101 | 0.204881738 | 0.351 | 0.104 | 1.10E−96 |
| B2m | 4.67E−101 | 0.246889569 | 0.997 | 0.963 | 1.16E−96 |
| Hebp1 | 2.97E−100 | −0.313009472 | 0.742 | 0.75 | 7.38E−96 |
| Sod1 | 3.76E−100 | −0.238388169 | 0.998 | 0.983 | 9.33E−96 |
| Man2a1 | 3.97E−100 | 0.369999358 | 0.83 | 0.583 | 9.87E−96 |
| mt-Nd6 | 5.56E−100 | −0.515107457 | 0.389 | 0.577 | 1.38E−95 |
| Hint2 | 6.90E−100 | −0.299309844 | 0.685 | 0.676 | 1.71E−95 |
| Map3k5 | 9.44E−100 | 0.21643947 | 0.411 | 0.147 | 2.34E−95 |
| Ndufb6 | 1.29E−99 | −0.311202811 | 0.77 | 0.759 | 3.20E−95 |
| Palmd | 2.20E−99 | 0.284673594 | 0.51 | 0.219 | 5.47E−95 |
| Eef2 | 2.45E−99 | 0.289562847 | 0.979 | 0.873 | 6.07E−95 |
| Vtn | 7.94E−99 | 0.225160672 | 0.999 | 0.983 | 1.97E−94 |
| Mgll | 8.46E−99 | 0.321840174 | 0.739 | 0.449 | 2.10E−94 |
| Aff4 | 2.96E−98 | 0.344186222 | 0.635 | 0.337 | 7.35E−94 |
| Aidh1l1 | 5.72E−98 | 0.277372649 | 0.991 | 0.904 | 1.42E−93 |
| Wdr45b | 7.36E−98 | 0.246836045 | 0.459 | 0.181 | 1.83E−93 |
| Gpd2 | 4.41E−97 | 0.23284812 | 0.393 | 0.135 | 1.10E−92 |
| Atp2a2 | 5.61E−97 | 0.350363266 | 0.771 | 0.495 | 1.39E−92 |
| Gadd45a | 2.31E−96 | 0.231268974 | 0.41 | 0.149 | 5.73E−92 |
| Mbnl2 | 3.16E−96 | 0.249293642 | 0.444 | 0.172 | 7.84E−92 |
| Eif1a | 3.45E−96 | 0.295219945 | 0.583 | 0.289 | 8.58E−92 |
| Igf1 | 4.40E−96 | 0.277138761 | 0.989 | 0.904 | 1.09E−91 |
| Acaa1b | 4.51E−96 | −0.263859024 | 0.968 | 0.969 | 1.12E−91 |
| Slc30a5 | 6.46E−96 | 0.207315427 | 0.378 | 0.126 | 1.60E−91 |
| Cyb5r3 | 1.19E−95 | 0.337684684 | 0.906 | 0.725 | 2.97E−91 |
| Il13ra1 | 1.45E−95 | 0.214635625 | 0.402 | 0.143 | 3.59E−91 |
| Slc3a1 | 8.64E−95 | 0.290117272 | 0.464 | 0.189 | 2.15E−90 |
| Serpina1e | 1.78E−94 | 0.233787997 | 1 | 0.999 | 4.43E−90 |
| Nop58 | 2.05E−94 | 0.257566653 | 0.428 | 0.162 | 5.09E−90 |
| Gm2a | 2.88E−94 | 0.341145926 | 0.704 | 0.427 | 7.15E−90 |
| Mvd | 3.47E−94 | −0.327327327 | 0.085 | 0.269 | 8.62E−90 |
| Por | 5.01E−94 | 0.290745567 | 0.898 | 0.675 | 1.24E−89 |
| Sparcl1 | 7.09E−94 | −0.253616986 | 0 | 0.136 | 1.76E−89 |
| Rpl13a | 2.36E−93 | −0.314606263 | 0.858 | 0.868 | 5.86E−89 |
| Enpp2 | 4.99E−93 | 0.316882991 | 0.635 | 0.345 | 1.24E−88 |
| Mpc2 | 5.01E−93 | −0.268047152 | 0.887 | 0.857 | 1.24E−88 |
| Ambp | 1.48E−92 | 0.211238054 | 0.999 | 0.993 | 3.68E−88 |
| Cyp1a2 | 2.74E−92 | −0.481919967 | 0.685 | 0.794 | 6.81E−88 |
| Dstn | 2.99E−92 | 0.271381717 | 0.664 | 0.37 | 7.43E−88 |
| Sec14l4 | 1.89E−91 | 0.285169227 | 0.724 | 0.436 | 4.69E−87 |
| Slc38a3 | 3.88E−91 | 0.292400046 | 0.971 | 0.858 | 9.64E−87 |
| Xdh | 1.64E−90 | 0.27224472 | 0.541 | 0.258 | 4.07E−86 |
| Cfhr2 | 2.18E−90 | −0.246923339 | 0.949 | 0.868 | 5.42E−86 |
| Slc16a6 | 3.73E−90 | 0.21530975 | 0.251 | 0.054 | 9.27E−86 |
| Spin1 | 4.09E−90 | 0.232119983 | 0.478 | 0.206 | 1.02E−85 |
| Dusp1 | 1.05E−89 | 0.300628481 | 0.539 | 0.259 | 2.62E−85 |
| Ubc | 1.64E−89 | −0.273242853 | 0.907 | 0.872 | 4.07E−85 |
| Aadac | 2.86E−89 | −0.230855362 | 0.952 | 0.86 | 7.10E−85 |
| Txnrd1 | 3.72E−89 | 0.314979852 | 0.721 | 0.441 | 9.23E−85 |
| Serinc3 | 1.70E−88 | 0.293964974 | 0.713 | 0.433 | 4.23E−84 |
| Dbi | 2.25E−88 | 0.243173797 | 0.999 | 0.971 | 5.58E−84 |
| C8a | 4.69E−88 | 0.294064237 | 0.978 | 0.887 | 1.16E−83 |
| Eci2 | 1.71E−87 | 0.288159513 | 0.896 | 0.698 | 4.24E−83 |
| Chd1 | 1.87E−87 | 0.213257509 | 0.42 | 0.166 | 4.65E−83 |
| Elovl2 | 2.58E−87 | 0.285038606 | 0.912 | 0.699 | 6.42E−83 |
| Atf6 | 2.81E−87 | 0.280521162 | 0.553 | 0.273 | 6.99E−83 |
| Lrp6 | 3.24E−87 | 0.270740438 | 0.505 | 0.232 | 8.05E−83 |
| Sigmar1 | 3.24E−86 | −0.282364617 | 0.482 | 0.514 | 8.06E−82 |
| Myo18a | 4.13E−86 | 0.233717966 | 0.476 | 0.211 | 1.03E−81 |
| Gck | 6.71E−86 | 0.260705425 | 0.557 | 0.278 | 1.67E−81 |
| Creg1 | 7.37E−86 | −0.235698597 | 0.966 | 0.926 | 1.83E−81 |
| Atp5f1 | 1.54E−85 | −0.217555876 | 0.943 | 0.869 | 3.81E−81 |
| Ndufa6 | 1.99E−85 | −0.25272382 | 0.846 | 0.798 | 4.93E−81 |
| Id3 | 2.15E−85 | 0.399520373 | 0.688 | 0.426 | 5.34E−81 |
| Asl | 3.26E−85 | 0.297673772 | 0.934 | 0.787 | 8.09E−81 |
| Tgm2 | 7.71E−85 | 0.316575272 | 0.524 | 0.258 | 1.91E−80 |
| Cpb2 | 8.35E−85 | 0.280302458 | 0.959 | 0.813 | 2.07E−80 |
| Uroc1 | 2.12E−84 | 0.329587334 | 0.837 | 0.615 | 5.26E−80 |
| Aox3 | 2.49E−84 | 0.275388909 | 0.989 | 0.894 | 6.17E−80 |
| Ddi2 | 3.40E−84 | 0.302519699 | 0.642 | 0.365 | 8.45E−80 |
| Flnb | 1.82E−83 | 0.212710481 | 0.376 | 0.139 | 4.52E−79 |
| Arl4d | 2.11E−83 | −0.328520283 | 0.326 | 0.437 | 5.23E−79 |
| Fgf1 | 2.25E−83 | 0.287983029 | 0.647 | 0.369 | 5.60E−79 |
| H6pd | 1.67E−82 | 0.300492669 | 0.825 | 0.595 | 4.14E−78 |
| Ctsb | 5.86E−82 | 0.268200419 | 0.943 | 0.781 | 1.46E−77 |
| Ddx5 | 6.19E−82 | 0.251675045 | 0.778 | 0.515 | 1.54E−77 |
| Megf9 | 6.32E−82 | 0.211011671 | 0.401 | 0.158 | 1.57E−77 |
| Mttp | 7.25E−82 | 0.296651415 | 0.888 | 0.697 | 1.80E−77 |
| Il6st | 7.72E−82 | 0.262348153 | 0.544 | 0.273 | 1.92E−77 |
| Gm9843 | 1.32E−81 | −0.343302667 | 0.197 | 0.299 | 3.27E−77 |
| Gde1 | 2.63E−81 | 0.222485647 | 0.531 | 0.263 | 6.54E−77 |
| Trp53inp1 | 4.94E−81 | 0.269278401 | 0.582 | 0.308 | 1.23E−76 |
| Rmnd5a | 6.65E−81 | 0.273473023 | 0.628 | 0.352 | 1.65E−76 |
| Fads1 | 7.07E−81 | 0.267264464 | 0.889 | 0.663 | 1.76E−76 |
| Gm10250 | 8.47E−81 | −0.30688429 | 0.124 | 0.251 | 2.10E−76 |
| Nrp1 | 1.23E−80 | 0.268796793 | 0.518 | 0.253 | 3.05E−76 |
| Rbm47 | 1.71E−80 | 0.21054927 | 0.475 | 0.218 | 4.24E−76 |
| Lrp1 | 1.77E−80 | 0.310226628 | 0.831 | 0.591 | 4.39E−76 |
| C4bp | 2.36E−80 | 0.26668764 | 0.948 | 0.788 | 5.86E−76 |
| Prox1 | 2.55E−80 | 0.31520499 | 0.622 | 0.351 | 6.34E−76 |
| Cebpa | 2.62E−80 | −0.260692112 | 0.688 | 0.672 | 6.50E−76 |
| Angptl4 | 3.50E−80 | 0.315315425 | 0.838 | 0.629 | 8.70E−76 |
| Lbp | 9.96E−80 | 0.264248666 | 0.605 | 0.333 | 2.47E−75 |
| Hs6st1 | 1.18E−79 | 0.206647988 | 0.524 | 0.261 | 2.94E−75 |
| Dcxr | 1.24E−79 | −0.271339616 | 0.629 | 0.631 | 3.07E−75 |
| Zfp683 | 1.36E−79 | −0.421689112 | 0 | 0.116 | 3.38E−75 |
| Clu | 1.37E−79 | 0.2220984 | 0.995 | 0.958 | 3.40E−75 |
| Cox7b | 1.54E−79 | −0.304638563 | 0.91 | 0.878 | 3.82E−75 |
| Picalm | 1.72E−79 | 0.276902165 | 0.648 | 0.376 | 4.27E−75 |
| Acly | 1.84E−79 | 0.287663867 | 0.715 | 0.445 | 4.57E−75 |
| Malat1 | 2.78E−79 | −0.652623643 | 0.704 | 0.78 | 6.90E−75 |
| Elovl3 | 4.42E−79 | −0.25229278 | 0.838 | 0.827 | 1.10E−74 |
| Atp5h | 9.55E−79 | −0.351854327 | 0.688 | 0.682 | 2.37E−74 |
| Hs3st3b1 | 1.01E−78 | 0.242350838 | 0.501 | 0.241 | 2.50E−74 |
| Ppp2r5a | 3.76E−78 | −0.263513608 | 0.593 | 0.594 | 9.33E−74 |
| Azin1 | 4.10E−78 | 0.268891169 | 0.59 | 0.322 | 1.02E−73 |
| Tmed5 | 5.21E−78 | 0.296018777 | 0.74 | 0.492 | 1.29E−73 |
| Tbl1xr1 | 8.70E−78 | 0.204315292 | 0.418 | 0.176 | 2.16E−73 |
| Hist1h1c | 1.16E−77 | −0.331045753 | 0.24 | 0.372 | 2.88E−73 |
| Copa | 1.43E−77 | 0.251410027 | 0.633 | 0.363 | 3.55E−73 |
| Gnl3 | 4.69E−77 | 0.242175469 | 0.454 | 0.204 | 1.16E−72 |
| Bhmt | 5.22E−77 | −0.236483408 | 0.988 | 0.956 | 1.30E−72 |
| Sord | 8.74E−77 | −0.228716308 | 0.964 | 0.931 | 2.17E−72 |
| Pik3c2a | 2.22E−76 | 0.232920912 | 0.444 | 0.198 | 5.51E−72 |
| Sec14l2 | 3.93E−76 | −0.220330298 | 0.905 | 0.856 | 9.77E−72 |
| Smoc1 | 7.72E−76 | 0.212439914 | 0.49 | 0.238 | 1.92E−71 |
| Hipk3 | 1.02E−75 | 0.225614187 | 0.472 | 0.223 | 2.53E−71 |
| Hhex | 1.26E−75 | −0.283483187 | 0.229 | 0.307 | 3.12E−71 |
| Ppib | 1.31E−75 | −0.223880669 | 0.823 | 0.767 | 3.26E−71 |
| Ctsl | 1.52E−75 | 0.256520866 | 0.976 | 0.857 | 3.77E−71 |
| Iqgap2 | 2.67E−75 | 0.283192936 | 0.879 | 0.677 | 6.63E−71 |
| Acsl4 | 3.34E−75 | 0.250793464 | 0.588 | 0.323 | 8.30E−71 |
| Ddc | 5.23E−75 | −0.216024433 | 0.475 | 0.46 | 1.30E−70 |
| Sc4mol | 1.02E−74 | −0.23053256 | 0.669 | 0.642 | 2.54E−70 |
| Slc38a4 | 1.12E−74 | 0.244607846 | 0.976 | 0.877 | 2.78E−70 |
| Lrpprc | 1.15E−74 | 0.257016288 | 0.662 | 0.398 | 2.84E−70 |
| Lurap1l | 1.62E−74 | 0.241965054 | 0.563 | 0.301 | 4.03E−70 |
| Cyp2c70 | 3.14E−74 | 0.25600888 | 0.971 | 0.834 | 7.79E−70 |
| Hsd3b3 | 5.05E−74 | −0.226222481 | 0.631 | 0.605 | 1.26E−69 |
| Gstm3 | 1.28E−73 | −0.470077922 | 0.343 | 0.414 | 3.17E−69 |
| Ass1 | 1.92E−73 | 0.216965784 | 0.996 | 0.962 | 4.76E−69 |
| Cfi | 2.17E−73 | 0.226479679 | 0.988 | 0.905 | 5.38E−69 |
| Ppp1r3b | 5.81E−73 | 0.259418401 | 0.642 | 0.38 | 1.44E−68 |
| Arhgef12 | 7.47E−73 | 0.280147378 | 0.667 | 0.408 | 1.85E−68 |
| Usp9x | 1.10E−72 | 0.233121474 | 0.556 | 0.299 | 2.72E−68 |
| Ranbp2 | 1.47E−72 | 0.208744477 | 0.596 | 0.338 | 3.64E−68 |
| Fbp1 | 1.75E−72 | 0.236214341 | 0.997 | 0.938 | 4.33E−68 |
| Ndufc2 | 1.98E−72 | −0.202304309 | 0.639 | 0.579 | 4.91E−68 |
| Nr2f6 | 2.24E−72 | −0.219631094 | 0.417 | 0.434 | 5.57E−68 |
| Ywhag | 6.54E−72 | 0.215838932 | 0.563 | 0.308 | 1.62E−67 |
| Dpy19l1 | 7.13E−72 | 0.23352829 | 0.531 | 0.277 | 1.77E−67 |
| Srpr | 7.66E−72 | 0.223990237 | 0.647 | 0.387 | 1.90E−67 |
| Cmah | 7.79E−72 | 0.247781083 | 0.721 | 0.465 | 1.93E−67 |
| Atp5j | 2.01E−71 | −0.234964444 | 0.782 | 0.757 | 5.00E−67 |
| Atp1a1 | 2.96E−71 | 0.261718912 | 0.709 | 0.457 | 7.36E−67 |
| Cyp2c55 | 5.49E−71 | −0.316565473 | 0.103 | 0.243 | 1.36E−66 |
| Calr | 5.63E−71 | 0.232562138 | 0.972 | 0.885 | 1.40E−66 |
| Fdx1 | 9.23E−71 | −0.213846254 | 0.669 | 0.625 | 2.29E−66 |
| Gls2 | 1.99E−70 | 0.296143664 | 0.868 | 0.657 | 4.94E−66 |
| Alas1 | 2.15E−70 | 0.283195713 | 0.905 | 0.729 | 5.33E−66 |
| Ftl1 | 2.87E−70 | −0.346327339 | 0.394 | 0.409 | 7.14E−66 |
| Pklr | 3.63E−70 | 0.25984035 | 0.624 | 0.368 | 9.00E−66 |
| Hsd17b13 | 6.39E−70 | 0.351593878 | 0.907 | 0.74 | 1.59E−65 |
| Pnpla7 | 6.61E−70 | 0.251226201 | 0.807 | 0.572 | 1.64E−65 |
| Tprkb | 1.03E−69 | −0.254084951 | 0.508 | 0.528 | 2.56E−65 |
| Osbpl1a | 1.14E−69 | 0.247696779 | 0.721 | 0.473 | 2.84E−65 |
| Comt | 1.83E−69 | 0.220396792 | 0.99 | 0.931 | 4.54E−65 |
| Nucks1 | 2.40E−69 | 0.249253158 | 0.683 | 0.43 | 5.96E−65 |
| Pim3 | 2.64E−69 | 0.280137371 | 0.764 | 0.537 | 6.55E−65 |
| Cldn1 | 3.97E−69 | −0.246774022 | 0.21 | 0.306 | 9.86E−65 |
| Nek7 | 4.17E−69 | 0.204786835 | 0.517 | 0.271 | 1.03E−64 |
| P4hb | 5.11E−69 | 0.204526132 | 0.995 | 0.941 | 1.27E−64 |
| Tm9sf2 | 5.15E−69 | 0.208759009 | 0.616 | 0.362 | 1.28E−64 |
| Akr1c20 | 6.04E−69 | −0.230255116 | 0.563 | 0.564 | 1.50E−64 |
| Ldha | 6.21E−69 | 0.234882634 | 0.974 | 0.896 | 1.54E−64 |
| Mtus1 | 1.13E−68 | 0.268076242 | 0.674 | 0.42 | 2.80E−64 |
| Me1 | 1.63E−68 | 0.229105587 | 0.699 | 0.447 | 4.06E−64 |
| Ptplad1 | 1.74E−68 | 0.255273084 | 0.741 | 0.496 | 4.33E−64 |
| Cnbp | 6.67E−68 | −0.203726646 | 0.828 | 0.778 | 1.66E−63 |
| Prdx4 | 9.03E−68 | −0.205807709 | 0.653 | 0.607 | 2.24E−63 |
| Cyp4a12a | 1.79E−67 | 0.259776426 | 0.823 | 0.597 | 4.43E−63 |
| Hdlbp | 2.95E−67 | 0.267317937 | 0.894 | 0.713 | 7.33E−63 |
| Dhrs3 | 6.96E−67 | −0.21828148 | 0.451 | 0.465 | 1.73E−62 |
| Nedd4 | 1.42E−66 | 0.259978699 | 0.762 | 0.524 | 3.52E−62 |
| Rpl14 | 2.56E−66 | −0.261157069 | 0.559 | 0.574 | 6.35E−62 |
| Mafb | 2.56E−66 | 0.216162055 | 0.577 | 0.33 | 6.36E−62 |
| Aplp2 | 3.47E−66 | 0.234842456 | 0.844 | 0.625 | 8.61E−62 |
| Kif21a | 1.13E−65 | 0.240302185 | 0.537 | 0.293 | 2.82E−61 |
| Macf1 | 1.69E−65 | 0.203727526 | 0.447 | 0.219 | 4.19E−61 |
| Myo1b | 2.38E−65 | 0.247482254 | 0.776 | 0.542 | 5.91E−61 |
| Uba1 | 3.31E−65 | 0.23027391 | 0.662 | 0.416 | 8.22E−61 |
| Kmo | 9.11E−65 | 0.262269388 | 0.891 | 0.732 | 2.26E−60 |
| Copz1 | 9.86E−65 | 0.221023165 | 0.631 | 0.385 | 2.45E−60 |
| Slc2a2 | 1.09E−64 | 0.218249447 | 0.722 | 0.479 | 2.70E−60 |
| Efna1 | 2.10E−64 | 0.256152989 | 0.551 | 0.309 | 5.22E−60 |
| Lpin2 | 2.47E−64 | 0.232988713 | 0.817 | 0.597 | 6.13E−60 |
| Ak3 | 2.57E−64 | 0.224559231 | 0.893 | 0.7 | 6.38E−60 |
| Agpat6 | 3.96E−64 | 0.242639639 | 0.749 | 0.517 | 9.83E−60 |
| Rcl1 | 1.06E−63 | 0.273905375 | 0.734 | 0.514 | 2.63E−59 |
| Ndufb4 | 1.26E−63 | −0.233018121 | 0.486 | 0.49 | 3.13E−59 |
| Zfp36l1 | 7.83E−63 | 0.271823824 | 0.636 | 0.402 | 1.95E−58 |
| D4Wsu53e | 3.61E−62 | −0.201263472 | 0.393 | 0.385 | 8.97E−58 |
| Wdr26 | 1.03E−61 | 0.207089713 | 0.646 | 0.406 | 2.55E−57 |
| Nr1i3 | 1.86E−61 | −0.317940692 | 0.294 | 0.377 | 4.63E−57 |
| Coa6 | 2.21E−61 | −0.242879305 | 0.344 | 0.373 | 5.49E−57 |
| Mup1 | 2.62E−61 | −0.490972296 | 0.609 | 0.583 | 6.50E−57 |
| Vkorc1 | 3.89E−61 | −0.206999378 | 0.545 | 0.54 | 9.65E−57 |
| Minos1 | 7.23E−61 | −0.201913176 | 0.638 | 0.595 | 1.80E−56 |
| Mir692-1 | 9.07E−61 | −0.20556011 | 0.012 | 0.126 | 2.25E−56 |
| Dusp6 | 9.26E−61 | 0.205655249 | 0.491 | 0.261 | 2.30E−56 |
| Slco1b2 | 1.29E−60 | −0.207101647 | 0.976 | 0.951 | 3.21E−56 |
| Cldn3 | 4.13E−60 | −0.24877302 | 0.273 | 0.355 | 1.02E−55 |
| Csde1 | 4.27E−60 | 0.209918785 | 0.797 | 0.577 | 1.06E−55 |
| Cyp3a59 | 1.17E−59 | −0.263118105 | 0.281 | 0.344 | 2.91E−55 |
| Mpc1 | 1.33E−59 | −0.205050162 | 0.349 | 0.369 | 3.31E−55 |
| 4931406C07Rik | 1.39E−59 | −0.233221778 | 0.789 | 0.748 | 3.45E−55 |
| Eif4g1 | 7.29E−59 | 0.201528669 | 0.865 | 0.669 | 1.81E−54 |
| Rab1 | 8.62E−59 | 0.211939923 | 0.673 | 0.438 | 2.14E−54 |
| Cyp3a16 | 9.45E−59 | −0.229013809 | 0.05 | 0.17 | 2.35E−54 |
| Dio1 | 5.55E−58 | −0.238782365 | 0.544 | 0.542 | 1.38E−53 |
| Cltc | 7.51E−58 | 0.238358233 | 0.647 | 0.414 | 1.86E−53 |
| Mrps18c | 8.71E−58 | −0.211258064 | 0.403 | 0.394 | 2.16E−53 |
| Dpyd | 1.40E−57 | 0.207189814 | 0.857 | 0.659 | 3.48E−53 |
| Reep3 | 2.41E−57 | 0.201856133 | 0.654 | 0.422 | 5.99E−53 |
| Hsdl2 | 2.60E−57 | 0.206098727 | 0.669 | 0.438 | 6.46E−53 |
| Gas5 | 2.71E−57 | −0.204205779 | 0.552 | 0.527 | 6.72E−53 |
| Tmem19 | 3.39E−57 | 0.214785487 | 0.693 | 0.465 | 8.41E−53 |
| Rps15 | 4.18E−57 | −0.213338023 | 0.581 | 0.528 | 1.04E−52 |
| Ogdh | 6.23E−57 | 0.220189108 | 0.601 | 0.37 | 1.55E−52 |
| Nr0b2 | 1.12E−56 | −0.203060823 | 0.227 | 0.28 | 2.79E−52 |
| Itih1 | 2.96E−56 | 0.213777239 | 0.946 | 0.816 | 7.34E−52 |
| Mcl1 | 3.89E−56 | 0.206406082 | 0.601 | 0.371 | 9.67E−52 |
| Pank1 | 1.20E−55 | 0.222539616 | 0.873 | 0.704 | 2.97E−51 |
| Phlda1 | 2.67E−55 | 0.201287476 | 0.583 | 0.356 | 6.63E−51 |
| 0610005C13Rik | 7.80E−55 | −0.2011928 | 0.677 | 0.653 | 1.94E−50 |
| Idh1 | 8.52E−55 | 0.208112098 | 0.921 | 0.766 | 2.12E−50 |
| 8430408G22Rik | 1.10E−54 | −0.221336883 | 0.336 | 0.392 | 2.73E−50 |
| Gdf15 | 3.32E−54 | 0.246982665 | 0.595 | 0.37 | 8.25E−50 |
| Gpd1 | 4.44E−54 | 0.200332863 | 0.856 | 0.665 | 1.10E−49 |
| Brd2 | 4.86E−54 | 0.258446853 | 0.629 | 0.408 | 1.21E−49 |
| Mup14 | 6.29E−54 | −0.340890047 | 0.699 | 0.616 | 1.56E−49 |
| Hc | 3.01E−53 | 0.223732951 | 0.976 | 0.903 | 7.48E−49 |
| Atp5j2 | 3.31E−53 | −0.218750887 | 0.887 | 0.852 | 8.22E−49 |
| Adh6-ps1 | 4.46E−53 | −0.22400228 | 0.259 | 0.313 | 1.11E−48 |
| Ubb | 8.42E−53 | −0.263773933 | 0.448 | 0.45 | 2.09E−48 |
| Dap | 1.08E−52 | 0.20987441 | 0.842 | 0.654 | 2.68E−48 |
| Gm24245 | 1.08E−52 | −0.254157233 | 0.192 | 0.214 | 2.68E−48 |
| Gstm1 | 4.17E−52 | −0.221666006 | 0.984 | 0.935 | 1.03E−47 |
| Atp5d | 5.05E−52 | 0.209976794 | 0.871 | 0.712 | 1.25E−47 |
| Ssr3 | 1.09E−51 | 0.214262703 | 0.796 | 0.596 | 2.71E−47 |
| Sdc1 | 1.41E−51 | 0.202649914 | 0.686 | 0.469 | 3.51E−47 |
| Pxmp4 | 1.72E−51 | 0.233212684 | 0.731 | 0.526 | 4.27E−47 |
| Blvrb | 2.50E−50 | −0.243251643 | 0.562 | 0.564 | 6.20E−46 |
| Rps18 | 2.68E−50 | −0.269206741 | 0.584 | 0.578 | 6.67E−46 |
| Fabp2 | 3.16E−50 | −0.226921259 | 0.631 | 0.622 | 7.84E−46 |
| Chchd2 | 4.37E−48 | −0.200916511 | 0.953 | 0.916 | 1.09E−43 |
| Cyp2f2 | 4.96E−48 | 0.275177176 | 0.965 | 0.863 | 1.23E−43 |
| F5 | 1.38E−47 | 0.202197412 | 0.886 | 0.728 | 3.44E−43 |
| Cyp2e1 | 2.57E−47 | −0.301497571 | 0.817 | 0.911 | 6.38E−43 |
| Rabac1 | 4.21E−47 | −0.211715679 | 0.522 | 0.524 | 1.05E−42 |
| Timm13 | 2.26E−46 | −0.204422177 | 0.695 | 0.671 | 5.61E−42 |
| Tsc22d1 | 1.34E−45 | −0.235196773 | 0.158 | 0.242 | 3.32E−41 |
| Cyp3a44 | 8.16E−45 | −0.205800676 | 0.164 | 0.183 | 2.03E−40 |
| Rnase4 | 2.92E−44 | 0.221993349 | 0.986 | 0.921 | 7.25E−40 |
| Fbxo21 | 8.37E−44 | −0.200640929 | 0.249 | 0.298 | 2.08E−39 |
| Iigp1 | 9.35E−44 | 0.232002466 | 0.895 | 0.759 | 2.32E−39 |
| Cyp4a10 | 1.19E−43 | −0.289941035 | 0.614 | 0.657 | 2.96E−39 |
| Gadd45b | 8.19E−40 | −0.219990566 | 0.254 | 0.291 | 2.03E−35 |
| Ifi27 | 4.76E−36 | 0.228184801 | 0.632 | 0.453 | 1.18E−31 |
| Mup20 | 2.24E−32 | −0.244695365 | 1 | 1 | 5.57E−28 |
| Lect2 | 1.65E−31 | −0.274252442 | 0.297 | 0.414 | 4.10E−27 |
| Saa3 | 8.72E−29 | 0.298054041 | 0.195 | 0.087 | 2.17E−24 |
| Oat | 5.39E−28 | −0.255537891 | 0.502 | 0.611 | 1.34E−23 |
| Csad | 9.84E−28 | 0.204004276 | 0.872 | 0.744 | 2.44E−23 |
| Mup7 | 0.007896043 | −0.293978105 | 0.998 | 0.998 | 1 |

TABLE 5c
Differentially expressed genes between time point PHX3 FC.15 and untreated (UT).

| p_val | avg_logFC | pct.1 | pct.2 | p_val_adj | |
|---|---|---|---|---|---|
| Saa2 | 0 | 2.971862499 | 0.999 | 0.222 | 0 |
| Mt1 | 0 | 2.610854994 | 1 | 0.556 | 0 |
| Mt2 | 0 | 2.6060431 | 1 | 0.365 | 0 |
| Saa1 | 0 | 2.202156726 | 1 | 0.662 | 0 |
| Steap4 | 0 | 1.890015167 | 0.973 | 0.222 | 0 |
| Serpina3n | 0 | 1.759758939 | 0.992 | 0.574 | 0 |
| Lcn2 | 0 | 1.499141183 | 0.815 | 0.07 | 0 |
| Lpin1 | 0 | 1.327657625 | 0.94 | 0.31 | 0 |
| n-R5-8s1 | 0 | 1.289598504 | 0.805 | 0.031 | 0 |
| Igfbp1 | 0 | 1.288789902 | 0.99 | 0.717 | 0 |
| Cxcl1 | 0 | 1.230398019 | 0.919 | 0.314 | 0 |
| Scd1 | 0 | 1.081676999 | 0.998 | 0.901 | 0 |
| Gm26924 | 0 | 1.063463323 | 1 | 1 | 0 |
| Il1r1 | 0 | 0.998168131 | 0.8 | 0.167 | 0 |
| Fga | 0 | 0.981249724 | 1 | 0.997 | 0 |
| Gm24601 | 0 | 0.924568273 | 0.661 | 0.015 | 0 |
| Nnmt | 0 | 0.901052904 | 0.97 | 0.716 | 0 |
| Itih4 | 0 | 0.887186241 | 0.999 | 0.948 | 0 |
| St3gal5 | 0 | 0.882761517 | 0.915 | 0.49 | 0 |
| Tacc2 | 0 | 0.877623973 | 0.707 | 0.071 | 0 |
| Lrg1 | 0 | 0.870250525 | 0.981 | 0.736 | 0 |
| Fam134b | 0 | 0.865578177 | 0.876 | 0.405 | 0 |
| Tat | 0 | 0.857965136 | 0.998 | 0.903 | 0 |
| Saa4 | 0 | 0.854921603 | 0.981 | 0.749 | 0 |
| Thrsp | 0 | 0.836598219 | 0.98 | 0.627 | 0 |
| Slc39a14 | 0 | 0.831584936 | 0.849 | 0.343 | 0 |
| Lars2 | 0 | 0.816795688 | 1 | 0.994 | 0 |
| Fgg | 0 | 0.809322713 | 1 | 0.991 | 0 |
| Slc38a2 | 0 | 0.797899442 | 0.874 | 0.413 | 0 |
| Serpina3m | 0 | 0.779052745 | 0.961 | 0.722 | 0 |
| Hp | 0 | 0.774785279 | 1 | 0.999 | 0 |
| Fgb | 0 | 0.739519125 | 1 | 1 | 0 |
| Gm15564 | 0 | 0.735157137 | 0.999 | 0.956 | 0 |
| Serpina10 | 0 | 0.733655099 | 0.934 | 0.641 | 0 |
| Plin2 | 0 | 0.720463076 | 0.952 | 0.714 | 0 |
| Aass | 0 | 0.705615345 | 0.965 | 0.716 | 0 |
| Fmo5 | 0 | 0.690160801 | 0.991 | 0.837 | 0 |
| Fn1 | 0 | 0.684266092 | 0.983 | 0.821 | 0 |
| Fkbp5 | 0 | 0.663304836 | 0.665 | 0.117 | 0 |
| Slc25a47 | 0 | 0.635928777 | 0.994 | 0.927 | 0 |
| Mat1a | 0 | 0.623734998 | 0.999 | 0.966 | 0 |
| Aldob | 0 | 0.490200848 | 1 | 0.996 | 0 |
| Mup3 | 0 | −0.526410044 | 1 | 1 | 0 |
| Cyb5 | 0 | −0.71965743 | 0.992 | 0.983 | 0 |
| mt-Rnr1 | 0 | −0.872186844 | 1 | 1 | 0 |
| mt-Rnr2 | 0 | −0.873557854 | 1 | 1 | 0 |
| Mup11 | 0 | −0.976755523 | 0.996 | 1 | 0 |
| mt-Nd2 | 0 | −1.128776891 | 0.996 | 0.999 | 0 |
| mt-Nd1 | 0 | −1.136736863 | 0.999 | 1 | 0 |
| mt-Nd5 | 0 | −1.145550707 | 0.974 | 0.995 | 0 |
| mt-Co3 | 0 | −1.233961828 | 0.106 | 0.603 | 0 |
| Gm13775 | 0 | −1.255657303 | 0.205 | 0.761 | 0 |
| mt-Cytb | 0 | −1.296043506 | 0.993 | 0.999 | 0 |
| mt-Co2 | 0 | −1.341686736 | 0.1 | 0.629 | 0 |
| mt-Co1 | 0 | −1.381307687 | 0.986 | 0.999 | 0 |
| mt-Nd4 | 0 | −1.630375247 | 0.977 | 0.998 | 0 |
| Mup4 | 0 | −1.805301681 | 0.374 | 0.898 | 0 |
| Mup6 | 0 | −2.386597202 | 0.714 | 0.992 | 0 |
| Mup9 | 0 | −2.453596744 | 0.401 | 0.976 | 0 |
| Mup17 | 0 | −2.514472885 | 0.899 | 1 | 0 |
| Mup18 | 0 | −2.617739049 | 0.547 | 0.997 | 0 |
| Mup19 | 0 | −2.932276479 | 0.84 | 1 | 0 |
| Acox1 | 1.48219693752374e−323 | 0.540022158 | 0.998 | 0.966 | 3.68059143525895e−319 |
| Hnf4a | 7.4109846876187e−323 | 0.644195253 | 0.969 | 0.743 | 1.84029571762948e−318 |
| B3galt1 | 1.9812032398234e−321 | 0.788734938 | 0.634 | 0.163 | 4.91972388512946e−317 |
| Cyp2b10 | 2.53603896010312e−320 | −1.254972098 | 0.132 | 0.542 | 6.29749194572806e−316 |
| Mup5 | 4.3570167109802e−319 | −1.484560547 | 0.52 | 0.839 | 1.0819343896706e−314 |
| H3f3b | 9.60962621817683e−319 | 0.755567107 | 0.831 | 0.419 | 2.38626238249767e−314 |
| Btg2 | 8.06389468707594e−310 | 0.772686832 | 0.708 | 0.241 | 2.00E−305 |
| Sephs2 | 8.7749437218295e−310 | 0.618707003 | 0.974 | 0.761 | 2.18E−305 |
| Ankrd55 | 1.57E−296 | −1.464696038 | 0.374 | 0.696 | 3.90E−292 |
| Hspb8 | 1.87E−295 | 0.667847253 | 0.914 | 0.597 | 4.63E−291 |
| Slc7a2 | 5.58E−288 | 0.622326529 | 0.964 | 0.735 | 1.38E−283 |
| Cldn14 | 9.96E−284 | 0.511583526 | 0.505 | 0.076 | 2.47E−279 |
| C3 | 5.41E−282 | 0.482674202 | 0.999 | 0.99 | 1.34E−277 |
| Gjb2 | 7.14E−278 | 0.639507436 | 0.901 | 0.572 | 1.77E−273 |
| Rcan1 | 1.17E−277 | 0.509239866 | 0.522 | 0.085 | 2.90E−273 |
| Errfi1 | 8.92E−268 | 0.500850526 | 0.998 | 0.911 | 2.21E−263 |
| Pfkfb3 | 1.02E−265 | 0.426580733 | 0.421 | 0.039 | 2.53E−261 |
| Arhgef26 | 1.60E−264 | 0.568544702 | 0.67 | 0.203 | 3.96E−260 |
| Cyp2c29 | 3.59E−262 | −1.027267705 | 0.751 | 0.93 | 8.92E−258 |
| Egfr | 9.60E−255 | 0.555834247 | 0.989 | 0.809 | 2.39E−250 |
| Gsta3 | 1.42E−250 | −0.53784222 | 0.993 | 0.956 | 3.53E−246 |
| Bcl3 | 1.69E−248 | 0.419650143 | 0.479 | 0.074 | 4.19E−244 |
| Snx10 | 3.34E−247 | 0.558244633 | 0.63 | 0.188 | 8.29E−243 |
| Pck1 | 8.81E−247 | 0.43447474 | 0.999 | 0.987 | 2.19E−242 |
| Mup16 | 3.38E−246 | −0.848726713 | 0.816 | 0.934 | 8.40E−242 |
| Bach1 | 1.06E−243 | 0.533237374 | 0.589 | 0.151 | 2.63E−239 |
| Tifa | 8.54E−243 | 0.392339477 | 0.393 | 0.036 | 2.12E−238 |
| Hpx | 9.49E−242 | 0.460272423 | 1 | 0.997 | 2.36E−237 |
| Ugt2b5 | 8.09E−241 | −0.423085187 | 0.991 | 0.959 | 2.01E−236 |
| Etnk2 | 2.61E−240 | 0.592353201 | 0.918 | 0.655 | 6.47E−236 |
| 2010003K11Rik | 8.39E−239 | 0.372219971 | 0.412 | 0.046 | 2.08E−234 |
| Rapgef4 | 8.83E−238 | 0.588317735 | 0.836 | 0.436 | 2.19E−233 |
| Tiparp | 5.11E−233 | 0.489122153 | 0.485 | 0.087 | 1.27E−228 |
| Gpt2 | 3.36E−230 | 0.533331785 | 0.949 | 0.725 | 8.35E−226 |
| Slc25a25 | 7.55E−227 | 0.544465891 | 0.945 | 0.704 | 1.87E−222 |
| Mup10 | 7.12E−224 | 0.428268029 | 0.965 | 0.692 | 1.77E−219 |
| Txnip | 3.44E−223 | 0.552612237 | 0.741 | 0.297 | 8.55E−219 |
| Cyp2c50 | 1.01E−219 | −0.763912176 | 0.693 | 0.888 | 2.51E−215 |
| Xbp1 | 2.95E−219 | 0.539986 | 0.89 | 0.57 | 7.32E−215 |
| Upp2 | 5.05E−218 | 0.541753512 | 0.934 | 0.673 | 1.25E−213 |
| Mfsd2a | 1.53E−217 | 0.496351884 | 0.563 | 0.148 | 3.79E−213 |
| Ephb3 | 3.99E−217 | 0.188403248 | 0.244 | 0.001 | 9.91E−213 |
| Actb | 2.09E−216 | 0.456981443 | 0.99 | 0.886 | 5.19E−212 |
| mmu-mir-6236 | 7.20E−215 | 0.468866739 | 0.989 | 0.837 | 1.79E−210 |
| Fbf1 | 1.39E−212 | 0.298573909 | 0.336 | 0.027 | 3.44E−208 |
| Hsd3b5 | 2.37E−209 | −0.587528886 | 0.49 | 0.587 | 5.88E−205 |
| Trib1 | 2.88E−209 | 0.581754281 | 0.705 | 0.308 | 7.14E−205 |
| Arg1 | 5.85E−205 | 0.381616351 | 1 | 0.982 | 1.45E−200 |
| Plin5 | 2.84E−204 | 0.489257529 | 0.736 | 0.315 | 7.05E−200 |
| Plk3 | 4.75E−204 | 0.488016367 | 0.644 | 0.226 | 1.18E−199 |
| Mup13 | 1.41E−203 | −0.755608269 | 0.303 | 0.546 | 3.49E−199 |
| Mtss1 | 6.23E−203 | 0.552137981 | 0.812 | 0.454 | 1.55E−198 |
| Klf6 | 1.09E−202 | 0.3616937 | 0.346 | 0.034 | 2.71E−198 |
| Itih3 | 7.66E−202 | 0.469631651 | 0.988 | 0.89 | 1.90E−197 |
| Gm23935 | 3.35E−201 | 0.498066925 | 0.999 | 0.925 | 8.33E−197 |
| Mup21 | 2.63E−199 | −0.668770594 | 0.829 | 0.929 | 6.54E−195 |
| Adck3 | 7.31E−199 | −0.482940158 | 0.729 | 0.764 | 1.81E−194 |
| Cyp2c54 | 4.33E−198 | −0.712859584 | 0.491 | 0.759 | 1.08E−193 |
| Serpina3c | 2.44E−196 | −0.554657351 | 0.118 | 0.385 | 6.07E−192 |
| Slc10a2 | 2.81E−196 | 0.30563824 | 0.376 | 0.05 | 6.99E−192 |
| Egr1 | 3.87E−196 | 0.620480469 | 0.79 | 0.447 | 9.61E−192 |
| Sdc4 | 3.89E−196 | 0.43560504 | 0.986 | 0.867 | 9.65E−192 |
| Apcs | 4.78E−195 | 0.573439593 | 0.854 | 0.525 | 1.19E−190 |
| Atf3 | 8.99E−194 | 0.597274889 | 0.56 | 0.179 | 2.23E−189 |
| Hsd3b7 | 1.65E−191 | −0.451728474 | 0.795 | 0.798 | 4.11E−187 |
| Orm1 | 3.32E−190 | 0.463411533 | 0.993 | 0.952 | 8.25E−186 |
| Pzp | 7.87E−190 | 0.387351113 | 0.999 | 0.986 | 1.95E−185 |
| Glul | 9.26E−190 | 0.478908893 | 0.58 | 0.18 | 2.30E−185 |
| Prg4 | 6.96E−189 | 0.480504574 | 0.585 | 0.188 | 1.73E−184 |
| Fgl1 | 1.60E−188 | 0.49338252 | 0.961 | 0.784 | 3.96E−184 |
| Gpcpd1 | 3.68E−188 | 0.53780646 | 0.778 | 0.398 | 9.13E−184 |
| Klf10 | 4.93E−188 | 0.304779554 | 0.388 | 0.06 | 1.22E−183 |
| Rgs16 | 2.29E−187 | 0.438105488 | 0.482 | 0.113 | 5.69E−183 |
| Ptp4a1 | 3.26E−187 | 0.393137367 | 0.528 | 0.145 | 8.09E−183 |
| mt-Atp6 | 3.25E−186 | −0.852555757 | 0.138 | 0.452 | 8.08E−182 |
| Fam35a | 2.34E−184 | 0.470461753 | 0.642 | 0.246 | 5.82E−180 |
| Rhob | 2.95E−184 | 0.37207281 | 0.4 | 0.069 | 7.32E−180 |
| Mup15 | 7.32E−184 | −1.074409915 | 0.357 | 0.617 | 1.82E−179 |
| Cyp3a25 | 9.18E−184 | −0.41051207 | 0.928 | 0.919 | 2.28E−179 |
| Got1 | 2.20E−183 | 0.542891511 | 0.866 | 0.587 | 5.45E−179 |
| Ces1d | 8.21E−180 | −0.385155368 | 0.944 | 0.923 | 2.04E−175 |
| Shb | 1.13E−178 | 0.430613558 | 0.589 | 0.204 | 2.80E−174 |
| Sds | 5.46E−177 | 0.787094904 | 0.868 | 0.67 | 1.36E−172 |
| Abca1 | 4.14E−176 | 0.50506234 | 0.756 | 0.377 | 1.03E−171 |
| 1810055G02Rik | 7.48E−174 | 0.382584093 | 0.528 | 0.155 | 1.86E−169 |
| Stat3 | 9.83E−173 | 0.460515229 | 0.701 | 0.318 | 2.44E−168 |
| Akr1c6 | 3.47E−171 | −0.43492211 | 0.992 | 0.971 | 8.62E−167 |
| Apoe | 9.56E−171 | −0.26209828 | 1 | 1 | 2.37E−166 |
| Igfbp4 | 4.54E−170 | 0.387861679 | 0.986 | 0.866 | 1.13E−165 |
| Cdkn1a | 2.74E−169 | 0.449325471 | 0.572 | 0.195 | 6.81E−165 |
| Gfra1 | 5.93E−169 | 0.474694426 | 0.835 | 0.498 | 1.47E−164 |
| Gc | 1.32E−168 | 0.233764264 | 1 | 0.999 | 3.29E−164 |
| Nrg4 | 3.45E−168 | 0.246089001 | 0.306 | 0.034 | 8.57E−164 |
| Ivns1abp | 1.41E−166 | 0.46254094 | 0.688 | 0.315 | 3.49E−162 |
| Fh1 | 2.37E−165 | 0.471428771 | 0.915 | 0.648 | 5.89E−161 |
| Myo1e | 9.03E−164 | 0.399456672 | 0.551 | 0.182 | 2.24E−159 |
| Slc41a2 | 1.17E−163 | 0.3685439 | 0.482 | 0.134 | 2.92E−159 |
| Mup8 | 2.13E−163 | −0.793731191 | 0.111 | 0.373 | 5.28E−159 |
| Slc25a51 | 3.33E−163 | 0.372157664 | 0.522 | 0.161 | 8.26E−159 |
| Psen2 | 1.99E−162 | −0.419555606 | 0.749 | 0.774 | 4.95E−158 |
| Dak | 3.59E−162 | 0.481947664 | 0.867 | 0.563 | 8.92E−158 |
| Apoc4 | 3.42E−160 | −0.356086407 | 0.999 | 0.98 | 8.50E−156 |
| Arid5a | 1.19E−159 | 0.163384737 | 0.208 | 0.005 | 2.96E−155 |
| Fasn | 1.87E−159 | 0.467168076 | 0.719 | 0.345 | 4.65E−155 |
| Bhlhe40 | 9.36E−159 | 0.403249298 | 0.628 | 0.251 | 2.32E−154 |
| Tmem87b | 4.56E−156 | 0.288330587 | 0.415 | 0.096 | 1.13E−151 |
| Ddx3x | 1.73E−155 | 0.47100162 | 0.823 | 0.513 | 4.30E−151 |
| Ttr | 2.14E−155 | 0.248320061 | 1 | 1 | 5.31E−151 |
| Aldh1a1 | 7.53E−152 | −0.40245097 | 0.984 | 0.956 | 1.87E−147 |
| Slc25a22 | 2.20E−151 | 0.390931532 | 0.649 | 0.282 | 5.45E−147 |
| Rnf103 | 8.64E−150 | 0.438540394 | 0.701 | 0.35 | 2.15E−145 |
| Scara5 | 1.64E−149 | 0.221632911 | 0.324 | 0.054 | 4.07E−145 |
| B4galt1 | 3.67E−147 | 0.321380868 | 0.494 | 0.155 | 9.10E−143 |
| Hacl1 | 1.26E−146 | −0.332899987 | 0.842 | 0.797 | 3.14E−142 |
| Rnf125 | 1.44E−146 | −0.463320018 | 0.312 | 0.493 | 3.57E−142 |
| Gm26917 | 1.97E−145 | 0.46739621 | 0.523 | 0.181 | 4.89E−141 |
| Junb | 2.51E−145 | 0.458500682 | 0.788 | 0.47 | 6.24E−141 |
| Cps1 | 4.13E−145 | 0.336429448 | 0.999 | 0.971 | 1.03E−140 |
| Atp1b1 | 1.93E−144 | 0.424801231 | 0.567 | 0.226 | 4.80E−140 |
| Glud1 | 7.16E−144 | 0.341724783 | 0.991 | 0.905 | 1.78E−139 |
| D10Wsu102e | 2.65E−142 | 0.256022156 | 0.338 | 0.063 | 6.58E−138 |
| Hyou1 | 3.00E−142 | 0.431109873 | 0.731 | 0.385 | 7.45E−138 |
| Jmjd1c | 3.16E−142 | 0.39612601 | 0.516 | 0.178 | 7.84E−138 |
| Apoa5 | 7.36E−142 | 0.30259839 | 0.999 | 0.974 | 1.83E−137 |
| 4933426M11Rik | 8.78E−142 | 0.28624304 | 0.419 | 0.109 | 2.18E−137 |
| Scp2 | 4.38E−141 | 0.279911381 | 0.997 | 0.986 | 1.09E−136 |
| Jun | 2.16E−140 | 0.479246114 | 0.864 | 0.593 | 5.36E−136 |
| Mup12 | 3.83E−140 | −0.589618573 | 0.792 | 0.86 | 9.51E−136 |
| Cyp2a5 | 4.16E−140 | −0.938183249 | 0.278 | 0.555 | 1.03E−135 |
| Cpt1a | 7.83E−140 | 0.42860734 | 0.894 | 0.651 | 1.94E−135 |
| Pik3r1 | 1.29E−139 | 0.389193614 | 0.577 | 0.231 | 3.21E−135 |
| Apob | 4.48E−139 | 0.416824707 | 0.997 | 0.989 | 1.11E−134 |
| Hmox1 | 1.22E−137 | 0.169723562 | 0.22 | 0.016 | 3.03E−133 |
| Epas1 | 1.71E−136 | 0.38425941 | 0.646 | 0.297 | 4.25E−132 |
| Ugt2b36 | 7.63E−136 | −0.287193139 | 0.978 | 0.93 | 1.90E−131 |
| Mbd1 | 2.07E−135 | 0.348094524 | 0.507 | 0.176 | 5.15E−131 |
| Mup2 | 6.51E−135 | −0.650107006 | 0.161 | 0.392 | 1.62E−130 |
| Pik3ap1 | 9.38E−135 | 0.19440062 | 0.283 | 0.043 | 2.33E−130 |
| Foxq1 | 5.32E−134 | 0.22551931 | 0.377 | 0.096 | 1.32E−129 |
| Slc22a5 | 7.77E−134 | 0.185456618 | 0.32 | 0.065 | 1.93E−129 |
| Gadd45g | 2.21E−133 | 0.392031882 | 0.899 | 0.639 | 5.49E−129 |
| Fos | 5.81E−133 | 0.524733327 | 0.785 | 0.51 | 1.44E−128 |
| Bcl2l1 | 6.28E−133 | 0.280315334 | 0.426 | 0.122 | 1.56E−128 |
| Ptpn1 | 8.84E−133 | 0.268263225 | 0.402 | 0.107 | 2.20E−128 |
| Slc25a15 | 2.01E−132 | 0.402049896 | 0.931 | 0.738 | 4.98E−128 |
| Tdo2 | 6.78E−132 | 0.25156007 | 0.999 | 0.991 | 1.68E−127 |
| Rnd3 | 2.06E−131 | 0.285147682 | 0.375 | 0.091 | 5.12E−127 |
| Adh1 | 2.79E−131 | −0.295980789 | 0.995 | 0.96 | 6.92E−127 |
| Litaf | 5.60E−131 | 0.396007707 | 0.603 | 0.278 | 1.39E−126 |
| Gm5096 | 6.85E−131 | −0.399548106 | 0.364 | 0.468 | 1.70E−126 |
| Wbp1l | 8.48E−130 | 0.409138156 | 0.867 | 0.606 | 2.11E−125 |
| Grn | 5.01E−129 | 0.403315761 | 0.726 | 0.396 | 1.24E−124 |
| Tns1 | 5.99E−129 | 0.33981795 | 0.524 | 0.196 | 1.49E−124 |
| Cyp2c37 | 7.22E−129 | −0.662335734 | 0.314 | 0.608 | 1.79E−124 |
| Ahcy | 1.24E−128 | −0.375627724 | 0.918 | 0.916 | 3.07E−124 |
| Foxa3 | 4.50E−128 | 0.307992015 | 0.505 | 0.182 | 1.12E−123 |
| Qsox1 | 6.27E−128 | 0.378486857 | 0.925 | 0.707 | 1.56E−123 |
| Cth | 9.41E−128 | −0.308847665 | 0.898 | 0.838 | 2.34E−123 |
| Id2 | 2.73E−127 | 0.430147785 | 0.826 | 0.579 | 6.79E−123 |
| 1700017B05Rik | 6.88E−127 | 0.182766029 | 0.263 | 0.037 | 1.71E−122 |
| Myh9 | 9.03E−127 | 0.399128031 | 0.653 | 0.318 | 2.24E−122 |
| Cox6c | 5.60E−126 | −0.41128283 | 0.893 | 0.884 | 1.39E−121 |
| Raph1 | 2.99E−125 | 0.242670968 | 0.382 | 0.1 | 7.42E−121 |
| Ier2 | 4.77E−123 | 0.452598264 | 0.763 | 0.49 | 1.18E−118 |
| Cfh | 5.82E−123 | 0.312615767 | 0.991 | 0.897 | 1.44E−118 |
| Itih2 | 1.49E−122 | 0.336714211 | 0.984 | 0.884 | 3.70E−118 |
| Rsp14 | 1.37E−121 | −0.346340977 | 0.936 | 0.917 | 3.39E−117 |
| Ccnl1 | 1.51E−121 | 0.341338448 | 0.526 | 0.206 | 3.76E−117 |
| Cyp8b1 | 1.66E−121 | −0.364571013 | 0.714 | 0.745 | 4.13E−117 |
| Cox4i1 | 1.99E−121 | −0.288035158 | 0.992 | 0.958 | 4.94E−117 |
| Eef1a1 | 5.92E−121 | 0.340768186 | 0.967 | 0.85 | 1.47E−116 |
| Nedd4l | 2.66E−120 | 0.271827098 | 0.447 | 0.147 | 6.59E−116 |
| Inmt | 6.09E−120 | −0.308364158 | 0.966 | 0.884 | 1.51E−115 |
| Cep85l | 1.56E−119 | 0.202664771 | 0.347 | 0.089 | 3.89E−115 |
| Elovl5 | 6.36E−119 | 0.382267188 | 0.863 | 0.58 | 1.58E−114 |
| Zbtb16 | 7.15E−119 | 0.232600907 | 0.414 | 0.131 | 1.77E−114 |
| Il17ra | 2.90E−118 | 0.203529938 | 0.302 | 0.061 | 7.21E−114 |
| Hip1r | 3.46E−118 | 0.213858215 | 0.362 | 0.098 | 8.58E−114 |
| Plg | 4.59E−118 | 0.26619661 | 0.998 | 0.961 | 1.14E−113 |
| Gcc1 | 5.36E−118 | 0.175053461 | 0.258 | 0.04 | 1.33E−113 |
| Eif4g2 | 7.33E−118 | 0.384302798 | 0.905 | 0.678 | 1.82E−113 |
| Atg14 | 1.12E−117 | 0.231648357 | 0.369 | 0.1 | 2.77E−113 |
| Ppip5k2 | 1.68E−117 | 0.30299148 | 0.51 | 0.197 | 4.16E−113 |
| Ulk1 | 3.53E−117 | 0.297841785 | 0.499 | 0.189 | 8.77E−113 |
| Pon1 | 1.13E−116 | −0.302819466 | 0.936 | 0.906 | 2.81E−112 |
| Elovl6 | 1.25E−116 | 0.37962641 | 0.553 | 0.24 | 3.10E−112 |
| Il6ra | 6.00E−116 | 0.251696836 | 0.431 | 0.142 | 1.49E−111 |
| Adamts1 | 3.81E−115 | 0.15752777 | 0.202 | 0.018 | 9.45E−111 |
| Ndufb11 | 5.66E−115 | −0.36792318 | 0.722 | 0.735 | 1.40E−110 |
| Ets2 | 9.44E−114 | 0.183062901 | 0.285 | 0.056 | 2.34E−109 |
| Igfbp2 | 3.42E−113 | −0.348403589 | 0.886 | 0.885 | 8.50E−109 |
| Fads2 | 1.76E−112 | 0.323744791 | 0.592 | 0.273 | 4.38E−108 |
| Tnfrsf1a | 2.66E−112 | 0.346845029 | 0.551 | 0.244 | 6.61E−108 |
| Gcnt2 | 3.52E−112 | 0.221618104 | 0.333 | 0.082 | 8.73E−108 |
| Ugt2b34 | 3.85E−112 | −0.290735843 | 0.918 | 0.874 | 9.57E−108 |
| Ell2 | 5.46E−112 | 0.371745977 | 0.72 | 0.416 | 1.36E−107 |
| Ly6e | 5.67E−112 | 0.42022985 | 0.68 | 0.386 | 1.41E−107 |
| Rps2 | 1.37E−110 | −0.44250848 | 0.675 | 0.757 | 3.40E−106 |
| Epb4.1 | 3.33E−110 | 0.349430673 | 0.617 | 0.304 | 8.26E−106 |
| Dscr3 | 6.45E−110 | 0.180232898 | 0.339 | 0.09 | 1.60E−105 |
| Ptpn2 | 1.68E−109 | 0.225408583 | 0.354 | 0.097 | 4.16E−105 |
| Clpx | 7.87E−109 | −0.306093278 | 0.818 | 0.783 | 1.95E−104 |
| Zc3h13 | 8.62E−109 | 0.204542393 | 0.325 | 0.083 | 2.14E−104 |
| Rps27l | 4.06E−108 | −0.361138678 | 0.855 | 0.849 | 1.01E−103 |
| Dst | 5.39E−108 | 0.329905553 | 0.507 | 0.206 | 1.34E−103 |
| Atl2 | 1.07E−107 | 0.321873809 | 0.674 | 0.361 | 2.67E−103 |
| Gm4076 | 1.67E−107 | −0.342157966 | 0.007 | 0.184 | 4.16E−103 |
| Lpgat1 | 2.80E−107 | 0.390146943 | 0.776 | 0.502 | 6.94E−103 |
| Gm20594 | 2.89E−107 | −0.559577825 | 0.108 | 0.294 | 7.17E−103 |
| Hmgcs1 | 6.61E−107 | −0.258009688 | 0.778 | 0.703 | 1.64E−102 |
| Esd | 1.82E−106 | −0.302906103 | 0.794 | 0.784 | 4.52E−102 |
| Oaz1 | 2.34E−106 | −0.339004868 | 0.759 | 0.75 | 5.80E−102 |
| Pptc7 | 8.06E−106 | 0.227095018 | 0.412 | 0.141 | 2.00E−101 |
| Sult1a1 | 1.16E−105 | 0.371023537 | 0.899 | 0.707 | 2.89E−101 |
| Ctif | 2.20E−105 | 0.18604392 | 0.312 | 0.078 | 5.46E−101 |
| Nfil3 | 2.44E−105 | 0.338730712 | 0.617 | 0.309 | 6.05E−101 |
| Aqp8 | 4.48E−105 | −0.331182913 | 0.444 | 0.505 | 1.11E−100 |
| Gpx1 | 4.61E−105 | −0.222094695 | 1 | 0.999 | 1.15E−100 |
| Cyp3a11 | 5.24E−105 | −0.251219977 | 0.999 | 1 | 1.30E−100 |
| Socs3 | 1.50E−104 | 0.194515821 | 0.456 | 0.183 | 3.73E−100 |
| Cml1 | 2.50E−104 | −0.277339974 | 0.803 | 0.757 | 6.22E−100 |
| Apoa4 | 2.57E−104 | 0.609214916 | 0.604 | 0.339 | 6.38E−100 |
| Jak1 | 3.30E−104 | 0.362766687 | 0.648 | 0.342 | 8.21E−100 |
| Arid5b | 4.81E−104 | 0.232391653 | 0.472 | 0.186 | 1.19E−99 |
| Rasgef1b | 6.74E−104 | 0.174426927 | 0.279 | 0.06 | 1.67E−99 |
| Baiap2l1 | 8.59E−104 | 0.187103281 | 0.395 | 0.136 | 2.13E−99 |
| Rrs1 | 1.28E−103 | 0.167967739 | 0.314 | 0.082 | 3.18E−99 |
| Ralgapa2 | 2.35E−103 | 0.20791298 | 0.334 | 0.092 | 5.82E−99 |
| G6pc | 4.54E−103 | 0.341403933 | 0.948 | 0.752 | 1.13E−98 |
| Rlf | 6.60E−103 | 0.176204022 | 0.321 | 0.086 | 1.64E−98 |
| Ifitm3 | 2.12E−102 | 0.331673739 | 0.958 | 0.812 | 5.27E−98 |
| Zfp36 | 2.37E−102 | 0.407014847 | 0.776 | 0.522 | 5.90E−98 |
| Oaf | 5.88E−102 | −0.333766768 | 0.79 | 0.794 | 1.46E−97 |
| Mgst1 | 6.37E−102 | −0.322742924 | 1 | 0.997 | 1.58E−97 |
| Uqcrq | 9.98E−102 | −0.316451873 | 0.98 | 0.951 | 2.48E−97 |
| Garem | 4.50E−101 | 0.204881738 | 0.351 | 0.104 | 1.12E−96 |
| B2m | 5.10E−101 | 0.246889569 | 0.997 | 0.963 | 1.27E−96 |
| Hebp1 | 3.43E−100 | −0.313009472 | 0.742 | 0.75 | 8.51E−96 |
| Man2al | 4.10E−100 | 0.369999358 | 0.83 | 0.583 | 1.02E−95 |
| Sod1 | 4.17E−100 | −0.238388169 | 0.998 | 0.983 | 1.04E−95 |
| mt-Nd6 | 5.99E−100 | −0.515107457 | 0.389 | 0.577 | 1.49E−95 |
| Hint2 | 8.14E−100 | −0.299309844 | 0.685 | 0.676 | 2.02E−95 |
| Map3k5 | 9.57E−100 | 0.21643947 | 0.411 | 0.147 | 2.38E−95 |
| Ndufb6 | 1.46E−99 | −0.311202811 | 0.77 | 0.759 | 3.63E−95 |
| Palmd | 2.20E−99 | 0.284673594 | 0.51 | 0.219 | 5.47E−95 |
| Eef2 | 2.59E−99 | 0.289562847 | 0.979 | 0.873 | 6.43E−95 |
| 3110043O21Rik | 3.51E−99 | 0.171966272 | 0.282 | 0.065 | 8.72E−95 |
| Heatr1 | 4.14E−99 | 0.163145002 | 0.275 | 0.062 | 1.03E−94 |
| Mgll | 8.58E−99 | 0.321840174 | 0.739 | 0.449 | 2.13E−94 |
| Vtn | 8.69E−99 | 0.225160672 | 0.999 | 0.983 | 2.16E−94 |
| Aff4 | 2.98E−98 | 0.344186222 | 0.635 | 0.337 | 7.41E−94 |
| Aldh1l1 | 6.03E−98 | 0.277372649 | 0.991 | 0.904 | 1.50E−93 |
| Wdr45b | 7.37E−98 | 0.246836045 | 0.459 | 0.181 | 1.83E−93 |
| Kdm4a | 1.44E−97 | 0.172058145 | 0.3 | 0.077 | 3.57E−93 |
| Gpd2 | 4.44E−97 | 0.23284812 | 0.393 | 0.135 | 1.10E−92 |
| Atp2a2 | 5.73E−97 | 0.350363266 | 0.771 | 0.495 | 1.42E−92 |
| Gadd45a | 2.33E−96 | 0.231268974 | 0.41 | 0.149 | 5.79E−92 |
| Mbnl2 | 3.16E−96 | 0.249293642 | 0.444 | 0.172 | 7.86E−92 |
| Eif1a | 3.47E−96 | 0.295219945 | 0.583 | 0.289 | 8.63E−92 |
| Igf1 | 4.62E−96 | 0.277138761 | 0.989 | 0.904 | 1.15E−91 |
| Acaa1b | 4.81E−96 | −0.263859024 | 0.968 | 0.969 | 1.20E−91 |
| Slc30a5 | 6.51E−96 | 0.207315427 | 0.378 | 0.126 | 1.62E−91 |
| Cyb5r3 | 1.26E−95 | 0.337684684 | 0.906 | 0.725 | 3.12E−91 |
| Il13ral | 1.46E−95 | 0.214635625 | 0.402 | 0.143 | 3.62E−91 |
| Dopey2 | 3.25E−95 | 0.19521799 | 0.33 | 0.097 | 8.06E−91 |
| Slc3a1 | 8.66E−95 | 0.290117272 | 0.464 | 0.189 | 2.15E−90 |
| Serpina1e | 1.97E−94 | 0.233787997 | 1 | 0.999 | 4.90E−90 |
| Nop58 | 2.05E−94 | 0.257566653 | 0.428 | 0.162 | 5.10E−90 |
| Gm2a | 2.97E−94 | 0.341145926 | 0.704 | 0.427 | 7.38E−90 |
| Ell | 3.83E−94 | 0.161665745 | 0.291 | 0.076 | 9.52E−90 |
| Mvd | 4.11E−94 | −0.327327327 | 0.085 | 0.269 | 1.02E−89 |
| Por | 5.09E−94 | 0.290745567 | 0.898 | 0.675 | 1.26E−89 |
| Sparcl1 | 7.09E−94 | −0.253616986 | 0 | 0.136 | 1.76E−89 |
| Rpl13a | 2.58E−93 | −0.314606263 | 0.858 | 0.868 | 6.42E−89 |
| Enpp2 | 5.04E−93 | 0.316882991 | 0.635 | 0.345 | 1.25E−88 |
| Kng1 | 5.14E−93 | 0.194558698 | 1 | 0.991 | 1.28E−88 |
| Mpc2 | 5.50E−93 | −0.268047152 | 0.887 | 0.857 | 1.36E−88 |
| Cep110 | 7.61E−93 | 0.192096419 | 0.362 | 0.121 | 1.89E−88 |
| Creb3l2 | 1.57E−92 | 0.161215013 | 0.278 | 0.068 | 3.90E−88 |
| Ambp | 1.66E−92 | 0.211238054 | 0.999 | 0.993 | 4.13E−88 |
| Cyp1a2 | 2.87E−92 | −0.481919967 | 0.685 | 0.794 | 7.13E−88 |
| Dstn | 2.99E−92 | 0.271381717 | 0.664 | 0.37 | 7.43E−88 |
| Ahsg | 1.27E−91 | 0.171853168 | 1 | 1 | 3.15E−87 |
| Pfkfb2 | 1.64E−91 | 0.160198891 | 0.27 | 0.067 | 4.08E−87 |
| Sec14l4 | 1.90E−91 | 0.285169227 | 0.724 | 0.436 | 4.71E−87 |
| Slc38a3 | 4.07E−91 | 0.292400046 | 0.971 | 0.858 | 1.01E−86 |
| Xdh | 1.64E−90 | 0.27224472 | 0.541 | 0.258 | 4.07E−86 |
| Cfhr2 | 2.31E−90 | −0.246923339 | 0.949 | 0.868 | 5.75E−86 |
| Slc16a6 | 3.74E−90 | 0.21530975 | 0.251 | 0.054 | 9.29E−86 |
| Spin1 | 4.10E−90 | 0.232119983 | 0.478 | 0.206 | 1.02E−85 |
| Coq10b | 5.98E−90 | 0.153862213 | 0.313 | 0.093 | 1.48E−85 |
| Dusp1 | 1.06E−89 | 0.300628481 | 0.539 | 0.259 | 2.64E−85 |
| Ubc | 1.78E−89 | −0.273242853 | 0.907 | 0.872 | 4.42E−85 |
| Aadac | 3.01E−89 | −0.230855362 | 0.952 | 0.86 | 7.48E−85 |
| Txnrd1 | 3.75E−89 | 0.314979852 | 0.721 | 0.441 | 9.32E−85 |
| Serinc3 | 1.72E−88 | 0.293964974 | 0.713 | 0.433 | 4.27E−84 |
| Dbi | 2.41E−88 | 0.243173797 | 0.999 | 0.971 | 5.98E−84 |
| C8a | 4.89E−88 | 0.294064237 | 0.978 | 0.887 | 1.22E−83 |
| Eci2 | 1.76E−87 | 0.288159513 | 0.896 | 0.698 | 4.36E−83 |
| Chd1 | 1.89E−87 | 0.213257509 | 0.42 | 0.166 | 4.68E−83 |
| Elovl2 | 2.62E−87 | 0.285038606 | 0.912 | 0.699 | 6.50E−83 |
| Atf6 | 2.81E−87 | 0.280521162 | 0.553 | 0.273 | 6.99E−83 |
| Lrp6 | 3.24E−87 | 0.270740438 | 0.505 | 0.232 | 8.05E−83 |
| Sigmar1 | 4.00E−86 | −0.282364617 | 0.482 | 0.514 | 9.94E−82 |
| Myo18a | 4.15E−86 | 0.233717966 | 0.476 | 0.211 | 1.03E−81 |
| Gck | 6.71E−86 | 0.260705425 | 0.557 | 0.278 | 1.67E−81 |
| D5Ertd579e | 7.69E−86 | 0.197825601 | 0.328 | 0.104 | 1.91E−81 |
| Creg1 | 7.90E−86 | −0.235698597 | 0.966 | 0.926 | 1.96E−81 |
| Atp5f1 | 1.64E−85 | −0.217555876 | 0.943 | 0.869 | 4.08E−81 |
| Zfp445 | 1.92E−85 | 0.159662672 | 0.371 | 0.138 | 4.77E−81 |
| Ndufa6 | 2.18E−85 | −0.25272382 | 0.846 | 0.798 | 5.42E−81 |
| Id3 | 2.19E−85 | 0.399520373 | 0.688 | 0.426 | 5.45E−81 |
| Nfkbiz | 2.84E−85 | 0.157772695 | 0.249 | 0.059 | 7.06E−81 |
| Asl | 3.40E−85 | 0.297673772 | 0.934 | 0.787 | 8.43E−81 |
| Tgm2 | 7.84E−85 | 0.316575272 | 0.524 | 0.258 | 1.95E−80 |
| Cpb2 | 8.61E−85 | 0.280302458 | 0.959 | 0.813 | 2.14E−80 |
| Uroc1 | 2.18E−84 | 0.329587334 | 0.837 | 0.615 | 5.42E−80 |
| Aox3 | 2.55E−84 | 0.275388909 | 0.989 | 0.894 | 6.34E−80 |
| Ddi2 | 3.43E−84 | 0.302519699 | 0.642 | 0.365 | 8.51E−80 |
| Flnb | 1.83E−83 | 0.212710481 | 0.376 | 0.139 | 4.55E−79 |
| Fgf1 | 2.26E−83 | 0.287983029 | 0.647 | 0.369 | 5.61E−79 |
| Arl4d | 2.64E−83 | −0.328520283 | 0.326 | 0.437 | 6.55E−79 |
| Cobll1 | 3.37E−83 | 0.158603105 | 0.353 | 0.13 | 8.38E−79 |
| H6pd | 1.71E−82 | 0.300492669 | 0.825 | 0.595 | 4.25E−78 |
| Tsc22d2 | 1.99E−82 | 0.165988347 | 0.349 | 0.124 | 4.94E−78 |
| Ctsb | 6.03E−82 | 0.268200419 | 0.943 | 0.781 | 1.50E−77 |
| Ddx5 | 6.22E−82 | 0.251675045 | 0.778 | 0.515 | 1.54E−77 |
| Megf9 | 6.35E−82 | 0.211011671 | 0.401 | 0.158 | 1.58E−77 |
| Mttp | 7.49E−82 | 0.296651415 | 0.888 | 0.697 | 1.86E−77 |
| Il6st | 7.72E−82 | 0.262348153 | 0.544 | 0.273 | 1.92E−77 |
| Gm9843 | 1.66E−81 | −0.343302667 | 0.197 | 0.299 | 4.13E−77 |
| Gde1 | 2.64E−81 | 0.222485647 | 0.531 | 0.263 | 6.55E−77 |
| Slc35g1 | 3.46E−81 | 0.166162728 | 0.36 | 0.133 | 8.58E−77 |
| Trp53inp1 | 4.94E−81 | 0.269278401 | 0.582 | 0.308 | 1.23E−76 |
| Rmnd5a | 6.65E−81 | 0.273473023 | 0.628 | 0.352 | 1.65E−76 |
| Fads1 | 7.11E−81 | 0.267264464 | 0.889 | 0.663 | 1.77E−76 |
| Gm10250 | 1.09E−80 | −0.30688429 | 0.124 | 0.251 | 2.72E−76 |
| Nrp1 | 1.23E−80 | 0.268796793 | 0.518 | 0.253 | 3.05E−76 |
| Rbm47 | 1.71E−80 | 0.21054927 | 0.475 | 0.218 | 4.25E−76 |
| Lrp1 | 1.78E−80 | 0.310226628 | 0.831 | 0.591 | 4.43E−76 |
| C4bp | 2.42E−80 | 0.26668764 | 0.948 | 0.788 | 6.01E−76 |
| Prox1 | 2.57E−80 | 0.31520499 | 0.622 | 0.351 | 6.37E−76 |
| Cebpa | 2.97E−80 | −0.260692112 | 0.688 | 0.672 | 7.37E−76 |
| Angptl4 | 3.62E−80 | 0.315315425 | 0.838 | 0.629 | 8.99E−76 |
| Clip1 | 4.55E−80 | 0.166877376 | 0.438 | 0.196 | 1.13E−75 |
| Cish | 6.12E−80 | 0.172549219 | 0.302 | 0.093 | 1.52E−75 |
| Lbp | 9.99E−80 | 0.264248666 | 0.605 | 0.333 | 2.48E−75 |
| Hs6st1 | 1.19E−79 | 0.206647988 | 0.524 | 0.261 | 2.96E−75 |
| Zfp683 | 1.36E−79 | −0.421689112 | 0 | 0.116 | 3.38E−75 |
| Dcxr | 1.42E−79 | −0.271339616 | 0.629 | 0.631 | 3.54E−75 |
| Clu | 1.46E−79 | 0.2220984 | 0.995 | 0.958 | 3.62E−75 |
| Cox7b | 1.64E−79 | −0.304638563 | 0.91 | 0.878 | 4.07E−75 |
| Picalm | 1.73E−79 | 0.276902165 | 0.648 | 0.376 | 4.29E−75 |
| Acly | 1.84E−79 | 0.287663867 | 0.715 | 0.445 | 4.57E−75 |
| Malat1 | 2.85E−79 | −0.652623643 | 0.704 | 0.78 | 7.08E−75 |
| Elovl3 | 4.71E−79 | −0.25229278 | 0.838 | 0.827 | 1.17E−74 |
| Hs3st3b1 | 1.01E−78 | 0.242350838 | 0.501 | 0.241 | 2.50E−74 |
| Atp5h | 1.05E−78 | −0.351854327 | 0.688 | 0.682 | 2.61E−74 |
| Nit2 | 2.04E−78 | −0.196867848 | 0.62 | 0.577 | 5.06E−74 |
| Ipo7 | 2.49E−78 | 0.176153152 | 0.402 | 0.168 | 6.18E−74 |
| Uox | 3.63E−78 | 0.189986074 | 0.998 | 0.965 | 9.02E−74 |
| Azin1 | 4.11E−78 | 0.268891169 | 0.59 | 0.322 | 1.02E−73 |
| Ppp2r5a | 4.38E−78 | −0.263513608 | 0.593 | 0.594 | 1.09E−73 |
| Tmed5 | 5.30E−78 | 0.296018777 | 0.74 | 0.492 | 1.32E−73 |
| Tbl1xr1 | 8.75E−78 | 0.204315292 | 0.418 | 0.176 | 2.17E−73 |
| Hist1h1c | 1.42E−77 | −0.331045753 | 0.24 | 0.372 | 3.53E−73 |
| Copa | 1.43E−77 | 0.251410027 | 0.633 | 0.363 | 3.55E−73 |
| Gnl3 | 4.69E−77 | 0.242175469 | 0.454 | 0.204 | 1.16E−72 |
| Bhmt | 5.54E−77 | −0.236483408 | 0.988 | 0.956 | 1.38E−72 |
| Sord | 9.31E−77 | −0.228716308 | 0.964 | 0.931 | 2.31E−72 |
| Nr1d2 | 9.97E−77 | 0.159943118 | 0.411 | 0.179 | 2.48E−72 |
| Rhobtb1 | 1.53E−76 | 0.178172942 | 0.406 | 0.172 | 3.79E−72 |
| Pik3c2a | 2.22E−76 | 0.232920912 | 0.444 | 0.198 | 5.51E−72 |
| Sec14l2 | 4.24E−76 | −0.220330298 | 0.905 | 0.856 | 1.05E−71 |
| Ythdc1 | 5.44E−76 | 0.15157378 | 0.369 | 0.15 | 1.35E−71 |
| Smoc1 | 7.76E−76 | 0.212439914 | 0.49 | 0.238 | 1.93E−71 |
| Plec | 8.16E−76 | 0.197499812 | 0.424 | 0.186 | 2.03E−71 |
| Hipk3 | 1.02E−75 | 0.225614187 | 0.472 | 0.223 | 2.54E−71 |
| Ppib | 1.44E−75 | −0.223880669 | 0.823 | 0.767 | 3.57E−71 |
| Ctsl | 1.56E−75 | 0.256520866 | 0.976 | 0.857 | 3.88E−71 |
| Bag3 | 1.58E−75 | 0.187468684 | 0.376 | 0.148 | 3.92E−71 |
| Hhex | 1.66E−75 | −0.283483187 | 0.229 | 0.307 | 4.12E−71 |
| Angptl3 | 1.98E−75 | −0.18094369 | 0.993 | 0.944 | 4.92E−71 |
| Iqgap2 | 2.71E−75 | 0.283192936 | 0.879 | 0.677 | 6.73E−71 |
| Acsl4 | 3.34E−75 | 0.250793464 | 0.588 | 0.323 | 8.30E−71 |
| Nr1i2 | 4.36E−75 | 0.198788095 | 0.505 | 0.252 | 1.08E−70 |
| Clp1 | 5.19E−75 | 0.153048593 | 0.307 | 0.103 | 1.29E−70 |
| Ddc | 6.37E−75 | −0.216024433 | 0.475 | 0.46 | 1.58E−70 |
| Lrpprc | 1.15E−74 | 0.257016288 | 0.662 | 0.398 | 2.85E−70 |
| Sc4mol | 1.15E−74 | −0.23053256 | 0.669 | 0.642 | 2.86E−70 |
| Slc38a4 | 1.16E−74 | 0.244607846 | 0.976 | 0.877 | 2.89E−70 |
| Lurap1l | 1.62E−74 | 0.241965054 | 0.563 | 0.301 | 4.03E−70 |
| Akap11 | 2.01E−74 | 0.187807202 | 0.392 | 0.164 | 4.99E−70 |
| Cyp2c70 | 3.18E−74 | 0.25600888 | 0.971 | 0.834 | 7.90E−70 |
| Gstp1 | 5.43E−74 | 0.197542109 | 0.962 | 0.822 | 1.35E−69 |
| Hsd3b3 | 5.80E−74 | −0.226222481 | 0.631 | 0.605 | 1.44E−69 |
| Sec24a | 8.91E−74 | 0.188750112 | 0.504 | 0.255 | 2.21E−69 |
| Sepp1 | 1.07E−73 | 0.16148927 | 1 | 1 | 2.66E−69 |
| Gstm3 | 1.41E−73 | −0.470077922 | 0.343 | 0.414 | 3.51E−69 |
| Ass1 | 2.01E−73 | 0.216965784 | 0.996 | 0.962 | 5.00E−69 |
| Cfi | 2.24E−73 | 0.226479679 | 0.988 | 0.905 | 5.56E−69 |
| Ppp1r3b | 5.81E−73 | 0.259418401 | 0.642 | 0.38 | 1.44E−68 |
| Arhgef12 | 7.49E−73 | 0.280147378 | 0.667 | 0.408 | 1.86E−68 |
| Usp9x | 1.10E−72 | 0.233121474 | 0.556 | 0.299 | 2.72E−68 |
| Ranbp2 | 1.47E−72 | 0.208744477 | 0.596 | 0.338 | 3.65E−68 |
| Fbp1 | 1.80E−72 | 0.236214341 | 0.997 | 0.938 | 4.47E−68 |
| Ndufc2 | 2.25E−72 | −0.202304309 | 0.639 | 0.579 | 5.59E−68 |
| Trip11 | 2.44E−72 | 0.163424021 | 0.429 | 0.2 | 6.05E−68 |
| Nr2f6 | 2.86E−72 | −0.219631094 | 0.417 | 0.434 | 7.10E−68 |
| 2900026A02Rik | 4.55E−72 | 0.175166554 | 0.416 | 0.187 | 1.13E−67 |
| Rc3h1 | 4.74E−72 | 0.165519544 | 0.33 | 0.122 | 1.18E−67 |
| Ywhag | 6.56E−72 | 0.215838932 | 0.563 | 0.308 | 1.63E−67 |
| Dpy19l1 | 7.13E−72 | 0.23352829 | 0.531 | 0.277 | 1.77E−67 |
| Srpr | 7.66E−72 | 0.223990237 | 0.647 | 0.387 | 1.90E−67 |
| Cmah | 7.79E−72 | 0.247781083 | 0.721 | 0.465 | 1.93E−67 |
| Ssfa2 | 8.16E−72 | 0.162814615 | 0.354 | 0.141 | 2.03E−67 |
| Apoc3 | 1.24E−71 | 0.187850611 | 0.999 | 0.993 | 3.08E−67 |
| Atp5j | 2.23E−71 | −0.234964444 | 0.782 | 0.757 | 5.53E−67 |
| Atp1a1 | 2.98E−71 | 0.261718912 | 0.709 | 0.457 | 7.40E−67 |
| Ppp1r15a | 5.29E−71 | 0.157408911 | 0.336 | 0.13 | 1.31E−66 |
| Calr | 5.87E−71 | 0.232562138 | 0.972 | 0.885 | 1.46E−66 |
| Cyp2c55 | 6.60E−71 | −0.316565473 | 0.103 | 0.243 | 1.64E−66 |
| Fdx1 | 1.04E−70 | −0.213846254 | 0.669 | 0.625 | 2.58E−66 |
| Gls2 | 2.00E−70 | 0.296143664 | 0.868 | 0.657 | 4.98E−66 |
| Alas1 | 2.18E−70 | 0.283195713 | 0.905 | 0.729 | 5.42E−66 |
| Cdip1 | 3.17E−70 | 0.161158186 | 0.514 | 0.273 | 7.88E−66 |
| Ftl1 | 3.30E−70 | −0.346327339 | 0.394 | 0.409 | 8.20E−66 |
| Pklr | 3.63E−70 | 0.25984035 | 0.624 | 0.368 | 9.02E−66 |
| Tmem205 | 4.55E−70 | −0.196745573 | 0.978 | 0.934 | 1.13E−65 |
| Hsd17b13 | 6.48E−70 | 0.351593878 | 0.907 | 0.74 | 1.61E−65 |
| Pnpla7 | 6.64E−70 | 0.251226201 | 0.807 | 0.572 | 1.65E−65 |
| Ech1 | 9.17E−70 | −0.191877671 | 0.908 | 0.856 | 2.28E−65 |
| Osbpl1a | 1.15E−69 | 0.247696779 | 0.721 | 0.473 | 2.86E−65 |
| Tprkb | 1.21E−69 | −0.254084951 | 0.508 | 0.528 | 3.01E−65 |
| Arl8b | 1.47E−69 | 0.198760529 | 0.468 | 0.228 | 3.65E−65 |
| Comt | 1.92E−69 | 0.220396792 | 0.99 | 0.931 | 4.76E−65 |
| Nucks1 | 2.40E−69 | 0.249253158 | 0.683 | 0.43 | 5.97E−65 |
| Pim3 | 2.68E−69 | 0.280137371 | 0.764 | 0.537 | 6.66E−65 |
| Nek7 | 4.18E−69 | 0.204786835 | 0.517 | 0.271 | 1.04E−64 |
| Tm9sf2 | 5.16E−69 | 0.208759009 | 0.616 | 0.362 | 1.28E−64 |
| Cldn1 | 5.28E−69 | −0.246774022 | 0.21 | 0.306 | 1.31E−64 |
| P4hb | 5.34E−69 | 0.204526132 | 0.995 | 0.941 | 1.33E−64 |
| Ldha | 6.49E−69 | 0.234882634 | 0.974 | 0.896 | 1.61E−64 |
| Akr1c20 | 6.92E−69 | −0.230255116 | 0.563 | 0.564 | 1.72E−64 |
| Ripk1 | 7.80E−69 | 0.174862366 | 0.407 | 0.183 | 1.94E−64 |
| Ibtk | 1.11E−68 | 0.188149661 | 0.485 | 0.247 | 2.76E−64 |
| Mtus1 | 1.13E−68 | 0.268076242 | 0.674 | 0.42 | 2.80E−64 |
| Rdh7 | 1.26E−68 | −0.197994968 | 0.993 | 0.969 | 3.13E−64 |
| Me1 | 1.64E−68 | 0.229105587 | 0.699 | 0.447 | 4.06E−64 |
| Ptplad1 | 1.75E−68 | 0.255273084 | 0.741 | 0.496 | 4.35E−64 |
| Scarb2 | 6.47E−68 | 0.19345963 | 0.603 | 0.353 | 1.61E−63 |
| Cnbp | 7.25E−68 | −0.203726646 | 0.828 | 0.778 | 1.80E−63 |
| Hagh | 9.52E−68 | −0.157822564 | 0.933 | 0.858 | 2.36E−63 |
| Prdx4 | 1.02E−67 | −0.205807709 | 0.653 | 0.607 | 2.52E−63 |
| Cep85 | 1.30E−67 | 0.171686021 | 0.383 | 0.167 | 3.22E−63 |
| Cyp4a12a | 1.79E−67 | 0.259776426 | 0.823 | 0.597 | 4.45E−63 |
| Hdlbp | 3.00E−67 | 0.267317937 | 0.894 | 0.713 | 7.44E−63 |
| St3gal1 | 4.24E−67 | 0.192410856 | 0.454 | 0.221 | 1.05E−62 |
| Gpc4 | 4.64E−67 | 0.16318479 | 0.349 | 0.141 | 1.15E−62 |
| Dhrs3 | 8.37E−67 | −0.21828148 | 0.451 | 0.465 | 2.08E−62 |
| Nedd4 | 1.42E−66 | 0.259978699 | 0.762 | 0.524 | 3.53E−62 |
| Mafb | 2.56E−66 | 0.216162055 | 0.577 | 0.33 | 6.36E−62 |
| Rpl14 | 2.95E−66 | −0.261157069 | 0.559 | 0.574 | 7.34E−62 |
| Aplp2 | 3.47E−66 | 0.234842456 | 0.844 | 0.625 | 8.63E−62 |
| Cap1 | 5.72E−66 | 0.156999287 | 0.42 | 0.202 | 1.42E−61 |
| Nploc4 | 6.46E−66 | 0.164369249 | 0.436 | 0.214 | 1.60E−61 |
| Atl3 | 7.09E−66 | 0.191666389 | 0.506 | 0.27 | 1.76E−61 |
| Kif21a | 1.13E−65 | 0.240302185 | 0.537 | 0.293 | 2.82E−61 |
| Cmbl | 1.44E−65 | −0.198257983 | 0.724 | 0.678 | 3.56E−61 |
| Macf1 | 1.70E−65 | 0.203727526 | 0.447 | 0.219 | 4.22E−61 |
| Alas2 | 2.09E−65 | 0.17568703 | 0.586 | 0.344 | 5.20E−61 |
| Actr2 | 2.14E−65 | 0.170960453 | 0.638 | 0.394 | 5.33E−61 |
| Gsk3b | 2.28E−65 | 0.159364703 | 0.41 | 0.193 | 5.65E−61 |
| Pcm1 | 2.32E−65 | 0.153416983 | 0.343 | 0.142 | 5.76E−61 |
| Myo1b | 2.39E−65 | 0.247482254 | 0.776 | 0.542 | 5.93E−61 |
| Ddx21 | 2.58E−65 | 0.186649034 | 0.448 | 0.22 | 6.41E−61 |
| Apoa2 | 2.71E−65 | −0.174241915 | 1 | 1 | 6.74E−61 |
| Itpr1 | 2.94E−65 | 0.161076343 | 0.356 | 0.152 | 7.30E−61 |
| Uba1 | 3.32E−65 | 0.23027391 | 0.662 | 0.416 | 8.23E−61 |
| Rap1b | 3.35E−65 | 0.174034663 | 0.491 | 0.258 | 8.31E−61 |
| Kmo | 9.36E−65 | 0.262269388 | 0.891 | 0.732 | 2.33E−60 |
| Copz1 | 9.86E−65 | 0.221023165 | 0.631 | 0.385 | 2.45E−60 |
| Slc2a2 | 1.09E−64 | 0.218249447 | 0.722 | 0.479 | 2.70E−60 |
| Plbd2 | 1.20E−64 | 0.169456289 | 0.42 | 0.199 | 2.99E−60 |
| Ythdf3 | 1.72E−64 | 0.174850983 | 0.474 | 0.243 | 4.28E−60 |
| Efna1 | 2.10E−64 | 0.256152989 | 0.551 | 0.309 | 5.23E−60 |
| Lpin2 | 2.48E−64 | 0.232988713 | 0.817 | 0.597 | 6.15E−60 |
| Dnttip2 | 2.57E−64 | 0.166547988 | 0.418 | 0.199 | 6.37E−60 |
| Ak3 | 2.58E−64 | 0.224559231 | 0.893 | 0.7 | 6.42E−60 |
| Supt6 | 2.61E−64 | 0.166662973 | 0.338 | 0.137 | 6.48E−60 |
| Wasl | 3.52E−64 | 0.178274463 | 0.449 | 0.223 | 8.75E−60 |
| Cd1d1 | 3.89E−64 | −0.182746213 | 0.644 | 0.612 | 9.67E−60 |
| Gbe1 | 3.98E−64 | 0.183485273 | 0.577 | 0.338 | 9.89E−60 |
| Aypat6 | 3.99E−64 | 0.242639639 | 0.749 | 0.517 | 9.90E−60 |
| Nme1 | 4.59E−64 | −0.195199734 | 0.674 | 0.622 | 1.14E−59 |
| Snd1 | 6.72E−64 | 0.18406298 | 0.549 | 0.312 | 1.67E−59 |
| Rcl1 | 1.08E−63 | 0.273905375 | 0.734 | 0.514 | 2.68E−59 |
| Ndufb4 | 1.48E−63 | −0.233018121 | 0.486 | 0.49 | 3.67E−59 |
| Gpam | 3.53E−63 | 0.169852715 | 0.417 | 0.199 | 8.76E−59 |
| Zfp36l1 | 7.93E−63 | 0.271823824 | 0.636 | 0.402 | 1.97E−58 |
| Fgd6 | 2.48E−62 | 0.16919996 | 0.348 | 0.147 | 6.16E−58 |
| Larp4 | 2.62E−62 | 0.156912192 | 0.396 | 0.184 | 6.50E−58 |
| Atp13a3 | 4.01E−62 | 0.197384706 | 0.65 | 0.41 | 9.95E−58 |
| D4Wsu53e | 4.36E−62 | −0.201263472 | 0.393 | 0.385 | 1.08E−57 |
| Fndc3b | 5.07E−62 | 0.151940318 | 0.276 | 0.096 | 1.26E−57 |
| Slc27a2 | 9.23E−62 | 0.192264004 | 0.991 | 0.949 | 2.29E−57 |
| Wdr26 | 1.03E−61 | 0.207089713 | 0.646 | 0.406 | 2.56E−57 |
| Nrn1 | 1.22E−61 | −0.19828092 | 0.704 | 0.675 | 3.04E−57 |
| Gns | 1.29E−61 | 0.166751532 | 0.516 | 0.288 | 3.20E−57 |
| Pex11a | 1.58E−61 | 0.185588983 | 0.681 | 0.441 | 3.93E−57 |
| Nr1i3 | 2.15E−61 | −0.317940692 | 0.294 | 0.377 | 5.33E−57 |
| Enpep | 2.37E−61 | 0.192022994 | 0.374 | 0.166 | 5.87E−57 |
| Coa6 | 2.69E−61 | −0.242879305 | 0.344 | 0.373 | 6.69E−57 |
| Mup1 | 2.75E−61 | −0.490972296 | 0.609 | 0.583 | 6.84E−57 |
| Lrrc58 | 3.23E−61 | 0.196709613 | 0.597 | 0.359 | 8.03E−57 |
| Dync1h1 | 3.27E−61 | 0.187521298 | 0.428 | 0.21 | 8.11E−57 |
| Slc16a10 | 4.35E−61 | 0.194608601 | 0.547 | 0.315 | 1.08E−56 |
| Vkorc1 | 4.55E−61 | −0.206999378 | 0.545 | 0.54 | 1.13E−56 |
| Selk | 6.14E−61 | −0.155942939 | 0.545 | 0.486 | 1.53E−56 |
| Sfpq | 7.57E−61 | 0.159417591 | 0.425 | 0.211 | 1.88E−56 |
| Minos1 | 8.02E−61 | −0.201913176 | 0.638 | 0.595 | 1.99E−56 |
| Dusp6 | 9.26E−61 | 0.205655249 | 0.491 | 0.261 | 2.30E−56 |
| Mir692-1 | 9.43E−61 | −0.20556011 | 0.012 | 0.126 | 2.34E−56 |
| Slco1b2 | 1.34E−60 | −0.207101647 | 0.976 | 0.951 | 3.32E−56 |
| Hist1h2bc | 1.64E−60 | −0.197841473 | 0.811 | 0.777 | 4.08E−56 |
| Itch | 2.42E−60 | 0.151077267 | 0.399 | 0.195 | 6.01E−56 |
| Kif5b | 3.67E−60 | 0.150172912 | 0.53 | 0.303 | 9.11E−56 |
| Csde1 | 4.27E−60 | 0.209918785 | 0.797 | 0.577 | 1.06E−55 |
| Ptprk | 4.67E−60 | 0.158881882 | 0.354 | 0.156 | 1.16E−55 |
| Cldn3 | 5.09E−60 | −0.24877302 | 0.273 | 0.355 | 1.26E−55 |
| Rab6a | 6.65E−60 | 0.191200862 | 0.57 | 0.335 | 1.65E−55 |
| Tspan4 | 1.11E−59 | 0.181956703 | 0.451 | 0.23 | 2.76E−55 |
| Hao1 | 1.22E−59 | 0.196088416 | 0.924 | 0.758 | 3.02E−55 |
| Cyp3a59 | 1.41E−59 | −0.263118105 | 0.281 | 0.344 | 3.49E−55 |
| Birc6 | 1.47E−59 | 0.164860409 | 0.502 | 0.28 | 3.64E−55 |
| 4931406C07Rik | 1.48E−59 | −0.233221778 | 0.789 | 0.748 | 3.67E−55 |
| Alkbh5 | 1.49E−59 | 0.198532353 | 0.507 | 0.279 | 3.69E−55 |
| Mpc1 | 1.66E−59 | −0.205050162 | 0.349 | 0.369 | 4.13E−55 |
| Ccdc25 | 6.51E−59 | 0.153512231 | 0.493 | 0.271 | 1.62E−54 |
| Acacb | 6.52E−59 | 0.172458783 | 0.33 | 0.137 | 1.62E−54 |
| Eif4g1 | 7.31E−59 | 0.201528669 | 0.865 | 0.669 | 1.82E−54 |
| Rab1 | 8.62E−59 | 0.211939923 | 0.673 | 0.438 | 2.14E−54 |
| Cyp3a16 | 1.09E−58 | −0.229013809 | 0.05 | 0.17 | 2.70E−54 |
| Uqcrb | 1.79E−58 | −0.157541559 | 0.721 | 0.641 | 4.44E−54 |
| Ghr | 1.87E−58 | 0.197884255 | 0.963 | 0.833 | 4.63E−54 |
| Vgll4 | 1.91E−58 | 0.152050279 | 0.363 | 0.164 | 4.75E−54 |
| Psmb1 | 2.41E−58 | −0.18427868 | 0.822 | 0.759 | 5.97E−54 |
| Ebpl | 2.69E−58 | −0.155251391 | 0.48 | 0.451 | 6.68E−54 |
| F11 | 4.57E−58 | 0.183078168 | 0.512 | 0.287 | 1.13E−53 |
| Cpne3 | 5.54E−58 | 0.182285896 | 0.537 | 0.31 | 1.38E−53 |
| C8g | 5.92E−58 | −0.1570956 | 0.965 | 0.91 | 1.47E−53 |
| Akr1c19 | 6.19E−58 | −0.179403044 | 0.547 | 0.527 | 1.54E−53 |
| Dio1 | 6.28E−58 | −0.238782365 | 0.544 | 0.542 | 1.56E−53 |
| Cltc | 7.51E−58 | 0.238358233 | 0.647 | 0.414 | 1.87E−53 |
| Cct8 | 9.58E−58 | 0.156517607 | 0.674 | 0.445 | 2.38E−53 |
| Mrps18c | 1.03E−57 | −0.211258064 | 0.403 | 0.394 | 2.55E−53 |
| Dpyd | 1.41E−57 | 0.207189814 | 0.857 | 0.659 | 3.49E−53 |
| Tram1 | 1.71E−57 | 0.180903738 | 0.737 | 0.512 | 4.26E−53 |
| Sec31a | 2.22E−57 | 0.151339369 | 0.53 | 0.31 | 5.50E−53 |
| Reep3 | 2.41E−57 | 0.201856133 | 0.654 | 0.422 | 5.99E−53 |
| Hsdl2 | 2.60E−57 | 0.206098727 | 0.669 | 0.438 | 6.46E−53 |
| Eif4b | 3.01E−57 | 0.163112238 | 0.666 | 0.438 | 7.47E−53 |
| Gas5 | 3.06E−57 | −0.204205779 | 0.552 | 0.527 | 7.60E−53 |
| Tmem19 | 3.39E−57 | 0.214785487 | 0.693 | 0.465 | 8.43E−53 |
| Sf3b1 | 3.86E−57 | 0.196390125 | 0.637 | 0.406 | 9.58E−53 |
| Gys2 | 3.98E−57 | 0.152105722 | 0.582 | 0.358 | 9.87E−53 |
| Rps15 | 4.60E−57 | −0.213338023 | 0.581 | 0.528 | 1.14E−52 |
| Ogdh | 6.23E−57 | 0.220189108 | 0.601 | 0.37 | 1.55E−52 |
| Huwe1 | 7.01E−57 | 0.177152033 | 0.536 | 0.314 | 1.74E−52 |
| Slc25a1 | 8.35E−57 | 0.187178873 | 0.724 | 0.499 | 2.07E−52 |
| Naa50 | 9.32E−57 | 0.187968549 | 0.586 | 0.358 | 2.31E−52 |
| Nab1 | 1.31E−56 | 0.151021868 | 0.533 | 0.315 | 3.25E−52 |
| Nr0b2 | 1.48E−56 | −0.203060823 | 0.227 | 0.28 | 3.68E−52 |
| Pah | 1.72E−56 | 0.196282042 | 0.983 | 0.891 | 4.27E−52 |
| Itih1 | 3.00E−56 | 0.213777239 | 0.946 | 0.816 | 7.46E−52 |
| Vwa8 | 3.49E−56 | 0.184402682 | 0.534 | 0.311 | 8.66E−52 |
| Mcl1 | 3.89E−56 | 0.206406082 | 0.601 | 0.371 | 9.67E−52 |
| Acadl | 4.71E−56 | 0.192143091 | 0.819 | 0.612 | 1.17E−51 |
| Trp53inp2 | 4.88E−56 | 0.176936721 | 0.655 | 0.428 | 1.21E−51 |
| Chchd1 | 5.13E−56 | −0.178218308 | 0.469 | 0.438 | 1.27E−51 |
| Kpna1 | 5.35E−56 | 0.161541953 | 0.411 | 0.206 | 1.33E−51 |
| Ndufa4 | 6.33E−56 | −0.196233158 | 0.941 | 0.889 | 1.57E−51 |
| Rhou | 6.93E−56 | 0.192074068 | 0.559 | 0.333 | 1.72E−51 |
| Pcca | 7.47E−56 | 0.150589178 | 0.594 | 0.373 | 1.85E−51 |
| Arl5a | 9.28E−56 | 0.169926786 | 0.508 | 0.289 | 2.30E−51 |
| Clcn4-2 | 1.08E−55 | 0.160606741 | 0.492 | 0.276 | 2.68E−51 |
| Pank1 | 1.21E−55 | 0.222539616 | 0.873 | 0.704 | 3.01E−51 |
| Phlda1 | 2.67E−55 | 0.201287476 | 0.583 | 0.356 | 6.63E−51 |
| Ugt1a1 | 2.89E−55 | 0.183517826 | 0.502 | 0.283 | 7.18E−51 |
| Amfr | 2.93E−55 | 0.184512887 | 0.738 | 0.516 | 7.27E−51 |
| Nars | 2.97E−55 | 0.191856779 | 0.696 | 0.471 | 7.38E−51 |
| Hspa4 | 4.00E−55 | 0.171672026 | 0.728 | 0.506 | 9.93E−51 |
| Arf4 | 5.56E−55 | 0.159979368 | 0.58 | 0.36 | 1.38E−50 |
| 0610005C13Rik | 8.56E−55 | −0.2011928 | 0.677 | 0.653 | 2.13E−50 |
| Idh1 | 8.60E−55 | 0.208112098 | 0.921 | 0.766 | 2.13E−50 |
| Furin | 9.57E−55 | 0.191384876 | 0.643 | 0.417 | 2.38E−50 |
| Atp5a1 | 1.01E−54 | 0.174138529 | 0.984 | 0.895 | 2.50E−50 |
| Ddx17 | 1.12E−54 | 0.159893824 | 0.491 | 0.277 | 2.79E−50 |
| 8430408G22Rik | 1.33E−54 | −0.221336883 | 0.336 | 0.392 | 3.30E−50 |
| St3gal4 | 1.39E−54 | 0.159434184 | 0.456 | 0.244 | 3.44E−50 |
| Ndufb8 | 1.42E−54 | −0.158878768 | 0.79 | 0.746 | 3.53E−50 |
| Etf1 | 1.44E−54 | 0.198764917 | 0.636 | 0.409 | 3.58E−50 |
| Gdf15 | 3.33E−54 | 0.246982665 | 0.595 | 0.37 | 8.26E−50 |
| Gpd1 | 4.45E−54 | 0.200332863 | 0.856 | 0.665 | 1.11E−49 |
| Tlcd2 | 4.61E−54 | −0.168255558 | 0.563 | 0.536 | 1.14E−49 |
| Brd2 | 4.88E−54 | 0.258446853 | 0.629 | 0.408 | 1.21E−49 |
| Mup14 | 6.56E−54 | −0.340890047 | 0.699 | 0.616 | 1.63E−49 |
| Hc | 3.08E−53 | 0.223732951 | 0.976 | 0.903 | 7.65E−49 |
| Atp5j2 | 3.49E−53 | −0.218750887 | 0.887 | 0.852 | 8.66E−49 |
| Ifitm2 | 3.98E−53 | 0.199025687 | 0.568 | 0.347 | 9.88E−49 |
| Adh6-ps1 | 5.41E−53 | −0.22400228 | 0.259 | 0.313 | 1.34E−48 |
| Cp | 6.17E−53 | 0.17916098 | 0.993 | 0.943 | 1.53E−48 |
| Slc30a10 | 8.57E−53 | 0.166623301 | 0.442 | 0.238 | 2.13E−48 |
| Aars | 8.79E−53 | 0.199299553 | 0.699 | 0.479 | 2.18E−48 |
| Ubb | 9.48E−53 | −0.263773933 | 0.448 | 0.45 | 2.35E−48 |
| Dap | 1.08E−52 | 0.20987441 | 0.842 | 0.654 | 2.69E−48 |
| Gm24245 | 1.31E−52 | −0.254157233 | 0.192 | 0.214 | 3.25E−48 |
| Rpl35 | 1.57E−52 | −0.175144379 | 0.216 | 0.225 | 3.91E−48 |
| Ireb2 | 1.70E−52 | 0.163596475 | 0.536 | 0.321 | 4.21E−48 |
| Ugdh | 1.95E−52 | 0.175644914 | 0.808 | 0.604 | 4.84E−48 |
| Ywhab | 2.03E−52 | 0.186791118 | 0.656 | 0.434 | 5.04E−48 |
| Hgfac | 2.16E−52 | 0.181878452 | 0.66 | 0.438 | 5.37E−48 |
| Prdx1 | 2.84E−52 | −0.174743472 | 0.971 | 0.927 | 7.05E−48 |
| Capzb | 3.07E−52 | 0.162969545 | 0.561 | 0.344 | 7.63E−48 |
| Akr1c14 | 3.37E−52 | −0.173913844 | 0.758 | 0.707 | 8.37E−48 |
| Pdcd6ip | 3.67E−52 | 0.181372134 | 0.556 | 0.338 | 9.12E−48 |
| Sppl2a | 4.25E−52 | 0.169493485 | 0.769 | 0.56 | 1.05E−47 |
| Gstm1 | 4.25E−52 | −0.221666006 | 0.984 | 0.935 | 1.06E−47 |
| Atp5d | 5.14E−52 | 0.209976794 | 0.871 | 0.712 | 1.28E−47 |
| Man1a | 5.65E−52 | 0.190591338 | 0.799 | 0.595 | 1.40E−47 |
| Arcn1 | 6.44E−52 | 0.158407448 | 0.582 | 0.366 | 1.60E−47 |
| Narf | 6.90E−52 | 0.163638744 | 0.46 | 0.253 | 1.71E−47 |
| Ssr3 | 1.09E−51 | 0.214262703 | 0.796 | 0.596 | 2.72E−47 |
| Ubr4 | 1.10E−51 | 0.17740179 | 0.455 | 0.25 | 2.74E−47 |
| Fam195a | 1.24E−51 | −0.190271304 | 0.355 | 0.373 | 3.08E−47 |
| Ifrd1 | 1.27E−51 | 0.175156039 | 0.379 | 0.185 | 3.15E−47 |
| Sdc1 | 1.41E−51 | 0.202649914 | 0.686 | 0.469 | 3.51E−47 |
| Mrp63 | 1.68E−51 | −0.184375029 | 0.563 | 0.531 | 4.18E−47 |
| Pxmp4 | 1.74E−51 | 0.233212684 | 0.731 | 0.526 | 4.32E−47 |
| Nlrp12 | 1.79E−51 | 0.153172692 | 0.648 | 0.43 | 4.44E−47 |
| 1110008F13Rik | 2.27E−51 | −0.197564988 | 0.666 | 0.648 | 5.64E−47 |
| Uqcr11 | 3.71E−51 | −0.196760374 | 0.918 | 0.879 | 9.21E−47 |
| Gna12 | 4.75E−51 | −0.191090072 | 0.352 | 0.354 | 1.18E−46 |
| Gm4952 | 4.76E−51 | −0.180740052 | 0.551 | 0.533 | 1.18E−46 |
| Cat | 5.57E−51 | 0.151152692 | 0.998 | 0.971 | 1.38E−46 |
| Ppm1k | 6.46E−51 | −0.198117939 | 0.469 | 0.45 | 1.60E−46 |
| Hint1 | 7.66E−51 | −0.15108001 | 0.915 | 0.858 | 1.90E−46 |
| Slc35d1 | 1.45E−50 | 0.1666456 | 0.651 | 0.435 | 3.61E−46 |
| Sult1d1 | 1.50E−50 | −0.183796178 | 0.739 | 0.684 | 3.72E−46 |
| Bzw1 | 1.97E−50 | 0.162308789 | 0.558 | 0.344 | 4.89E−46 |
| Ces1e | 2.56E−50 | 0.166391723 | 0.779 | 0.576 | 6.36E−46 |
| Blvrb | 2.69E−50 | −0.243251643 | 0.562 | 0.564 | 6.69E−46 |
| Rps18 | 2.90E−50 | −0.269206741 | 0.584 | 0.578 | 7.20E−46 |
| Abca6 | 3.37E−50 | 0.198869605 | 0.719 | 0.507 | 8.36E−46 |
| Fabp2 | 3.39E−50 | −0.226921259 | 0.631 | 0.622 | 8.42E−46 |
| Apol7a | 3.48E−50 | −0.165242629 | 0.39 | 0.382 | 8.65E−46 |
| Nudt4 | 3.71E−50 | 0.183172596 | 0.773 | 0.568 | 9.21E−46 |
| Grlf1 | 4.39E−50 | 0.163008664 | 0.457 | 0.257 | 1.09E−45 |
| Son | 5.47E−50 | 0.188921988 | 0.573 | 0.358 | 1.36E−45 |
| Lgals9 | 5.77E−50 | 0.184003378 | 0.936 | 0.793 | 1.43E−45 |
| Rtfdc1 | 7.57E−50 | 0.16242752 | 0.627 | 0.412 | 1.88E−45 |
| Serbp1 | 1.02E−49 | 0.186271124 | 0.824 | 0.634 | 2.54E−45 |
| Pqlc1 | 1.31E−49 | −0.161639859 | 0.364 | 0.36 | 3.26E−45 |
| Osbpl9 | 1.67E−49 | 0.161985999 | 0.661 | 0.448 | 4.15E−45 |
| Ttc36 | 1.68E−49 | −0.175280872 | 0.983 | 0.95 | 4.17E−45 |
| Zbtb20 | 3.03E−49 | 0.197963292 | 0.657 | 0.445 | 7.53E−45 |
| Eprs | 3.05E−49 | 0.19400896 | 0.7 | 0.488 | 7.57E−45 |
| A1cf | 3.11E−49 | 0.163393145 | 0.66 | 0.449 | 7.72E−45 |
| Agt | 5.44E−49 | 0.169771558 | 0.941 | 0.817 | 1.35E−44 |
| Ubr2 | 6.83E−49 | 0.158348096 | 0.464 | 0.265 | 1.70E−44 |
| Lrrc59 | 6.85E−49 | 0.163232541 | 0.484 | 0.28 | 1.70E−44 |
| Aco2 | 9.03E−49 | 0.155048742 | 0.702 | 0.493 | 2.24E−44 |
| Pten | 1.24E−48 | 0.166403783 | 0.669 | 0.458 | 3.07E−44 |
| Cbs | 1.25E−48 | 0.199528185 | 0.901 | 0.749 | 3.10E−44 |
| Dhcr24 | 1.81E−48 | 0.165813919 | 0.932 | 0.783 | 4.49E−44 |
| Pygl | 2.29E−48 | 0.19507544 | 0.92 | 0.776 | 5.68E−44 |
| Prkar1a | 2.52E−48 | 0.165756705 | 0.67 | 0.46 | 6.26E−44 |
| Commd6 | 2.96E−48 | −0.168923514 | 0.391 | 0.39 | 7.36E−44 |
| Chchd2 | 4.53E−48 | −0.200916511 | 0.953 | 0.916 | 1.13E−43 |
| Cyp2f2 | 4.99E−48 | 0.275177176 | 0.965 | 0.863 | 1.24E−43 |
| Glol | 5.98E−48 | −0.154642914 | 0.804 | 0.749 | 1.49E−43 |
| Dnajc19 | 6.41E−48 | −0.156899631 | 0.412 | 0.387 | 1.59E−43 |
| Atxn7l3b | 7.88E−48 | 0.191387966 | 0.686 | 0.476 | 1.96E−43 |
| Col18a1 | 9.42E−48 | 0.159648412 | 0.934 | 0.79 | 2.34E−43 |
| Rpl36a1 | 1.01E−47 | −0.186625637 | 0.601 | 0.551 | 2.51E−43 |
| F5 | 1.39E−47 | 0.202197412 | 0.886 | 0.728 | 3.45E−43 |
| Ppp2ca | 1.60E−47 | 0.154757155 | 0.64 | 0.43 | 3.98E−43 |
| Paics | 1.64E−47 | 0.198478345 | 0.842 | 0.662 | 4.08E−43 |
| Gsta2 | 1.74E−47 | −0.188661153 | 0.296 | 0.333 | 4.33E−43 |
| Psma4 | 1.83E−47 | −0.153086391 | 0.544 | 0.495 | 4.55E−43 |
| Dnaja2 | 1.91E−47 | 0.160071794 | 0.612 | 0.402 | 4.75E−43 |
| Cyp2e1 | 2.58E−47 | −0.301497571 | 0.817 | 0.911 | 6.41E−43 |
| Nadk | 3.79E−47 | 0.15446959 | 0.661 | 0.454 | 9.40E−43 |
| Rabac1 | 4.67E−47 | −0.211715679 | 0.522 | 0.524 | 1.16E−42 |
| Slc31a1 | 8.37E−47 | 0.182041621 | 0.693 | 0.486 | 2.08E−42 |
| Sco2 | 8.41E−47 | −0.198692618 | 0.507 | 0.508 | 2.09E−42 |
| Mrpl41 | 1.76E−46 | −0.172436199 | 0.341 | 0.348 | 4.38E−42 |
| Gnas | 1.80E−46 | 0.159624116 | 0.947 | 0.82 | 4.47E−42 |
| AI182371 | 2.26E−46 | 0.179018804 | 0.81 | 0.622 | 5.61E−42 |
| Timm13 | 2.42E−46 | −0.204422177 | 0.695 | 0.671 | 6.02E−42 |
| Gchfr | 3.03E−46 | −0.152643272 | 0.833 | 0.768 | 7.51E−42 |
| Gyk | 3.44E−46 | 0.150378816 | 0.553 | 0.35 | 8.55E−42 |
| Etnk1 | 4.88E−46 | 0.15045907 | 0.527 | 0.328 | 1.21E−41 |
| Ndufs6 | 6.02E−46 | −0.195648216 | 0.771 | 0.724 | 1.49E−41 |
| 9430023L20Rik | 7.56E−46 | −0.164422856 | 0.264 | 0.282 | 1.88E−41 |
| Map1lc3b | 1.57E−45 | 0.167697241 | 0.602 | 0.395 | 3.89E−41 |
| Tsc22d1 | 1.58E−45 | −0.235196773 | 0.158 | 0.242 | 3.92E−41 |
| Clmn | 2.09E−45 | 0.159639031 | 0.509 | 0.31 | 5.20E−41 |
| Hdac11 | 2.63E−45 | −0.196507741 | 0.251 | 0.285 | 6.53E−41 |
| Edem1 | 2.95E−45 | 0.172255296 | 0.635 | 0.429 | 7.32E−41 |
| Fah | 3.84E−45 | 0.188483379 | 0.941 | 0.834 | 9.54E−41 |
| Gstt3 | 5.49E−45 | −0.159722425 | 0.296 | 0.286 | 1.36E−40 |
| Pbld1 | 5.50E−45 | −0.1818338 | 0.592 | 0.565 | 1.37E−40 |
| Gstm4 | 6.47E−45 | −0.157553603 | 0.478 | 0.444 | 1.61E−40 |
| Crp | 6.68E−45 | 0.178468102 | 0.905 | 0.756 | 1.66E−40 |
| Tgoln1 | 7.76E−45 | 0.176355766 | 0.768 | 0.575 | 1.93E−40 |
| Rps19 | 9.26E−45 | −0.151004347 | 0.562 | 0.474 | 2.30E−40 |
| Neat1 | 9.39E−45 | 0.164921803 | 0.293 | 0.135 | 2.33E−40 |
| Anp32a | 9.68E−45 | −0.154984115 | 0.436 | 0.413 | 2.40E−40 |
| Ndufb2 | 1.00E−44 | −0.154329774 | 0.685 | 0.629 | 2.49E−40 |
| Cyp3a44 | 1.00E−44 | −0.205800676 | 0.164 | 0.183 | 2.49E−40 |
| Ndufa2 | 1.04E−44 | −0.194549142 | 0.792 | 0.738 | 2.57E−40 |
| 1110058L19Rik | 1.49E−44 | −0.160260673 | 0.272 | 0.281 | 3.69E−40 |
| Surf4 | 1.87E−44 | 0.150111361 | 0.746 | 0.551 | 4.64E−40 |
| Hspa9 | 2.74E−44 | 0.158702904 | 0.947 | 0.824 | 6.81E−40 |
| Sdha | 2.84E−44 | 0.152056738 | 0.902 | 0.747 | 7.06E−40 |
| Rnase4 | 2.95E−44 | 0.221993349 | 0.986 | 0.921 | 7.33E−40 |
| Trip12 | 3.21E−44 | 0.158568104 | 0.544 | 0.345 | 7.98E−40 |
| Acadsb | 3.48E−44 | 0.161705277 | 0.79 | 0.601 | 8.65E−40 |
| Qprt | 5.28E−44 | 0.156064415 | 0.698 | 0.498 | 1.31E−39 |
| Afm | 7.47E−44 | 0.159606308 | 0.745 | 0.55 | 1.86E−39 |
| Iigp1 | 9.44E−44 | 0.232002466 | 0.895 | 0.759 | 2.34E−39 |
| Fbxo21 | 9.92E−44 | −0.200640929 | 0.249 | 0.298 | 2.46E−39 |
| Cyp4a10 | 1.24E−43 | −0.289941035 | 0.614 | 0.657 | 3.08E−39 |
| Hrsp12 | 1.65E−43 | −0.156961195 | 0.98 | 0.939 | 4.11E−39 |
| H2-D1 | 1.76E−43 | 0.167536811 | 0.916 | 0.774 | 4.38E−39 |
| Slc22a1 | 2.39E−43 | −0.199196167 | 0.777 | 0.747 | 5.94E−39 |
| Bche | 2.46E−43 | 0.162843566 | 0.566 | 0.369 | 6.11E−39 |
| Skp1a | 2.87E−43 | 0.172529381 | 0.791 | 0.606 | 7.13E−39 |
| Tspo | 4.64E−43 | −0.171115456 | 0.532 | 0.506 | 1.15E−38 |
| Pgrmc1 | 5.46E−43 | 0.159063843 | 0.99 | 0.94 | 1.36E−38 |
| Cyp2c44 | 9.90E−43 | 0.164710905 | 0.863 | 0.698 | 2.46E−38 |
| 1810022K09Rik | 1.34E−42 | −0.180096346 | 0.286 | 0.301 | 3.32E−38 |
| Kcnk5 | 2.83E−42 | −0.168374452 | 0.107 | 0.173 | 7.02E−38 |
| Selenbp1 | 4.91E−42 | −0.185040969 | 0.725 | 0.699 | 1.22E−37 |
| Fetub | 1.37E−41 | 0.174405959 | 0.859 | 0.703 | 3.41E−37 |
| Cox8a | 2.26E−41 | 0.176195798 | 0.796 | 0.621 | 5.62E−37 |
| Srp9 | 2.81E−41 | −0.160772329 | 0.333 | 0.336 | 6.97E−37 |
| Serping1 | 3.58E−41 | 0.153322882 | 0.963 | 0.87 | 8.90E−37 |
| Gstt2 | 5.06E−41 | −0.190732451 | 0.397 | 0.404 | 1.26E−36 |
| 2410015M20Rik | 5.10E−41 | −0.169256363 | 0.635 | 0.604 | 1.27E−36 |
| Pglyrp2 | 7.08E−41 | 0.150653064 | 0.607 | 0.414 | 1.76E−36 |
| Mug2 | 9.73E−41 | 0.179746266 | 0.887 | 0.738 | 2.42E−36 |
| Rpl32 | 1.01E−40 | −0.190948597 | 0.8 | 0.757 | 2.51E−36 |
| Tmem106b | 3.79E−40 | 0.153891421 | 0.743 | 0.557 | 9.40E−36 |
| Tomm70a | 4.81E−40 | 0.151256378 | 0.685 | 0.494 | 1.19E−35 |
| Mettl20 | 4.88E−40 | −0.163343248 | 0.311 | 0.327 | 1.21E−35 |
| Gadd45b | 9.33E−40 | −0.219990566 | 0.254 | 0.291 | 2.32E−35 |
| S100a10 | 1.12E−39 | −0.177702028 | 0.676 | 0.662 | 2.79E−35 |
| Htatip2 | 1.90E−39 | −0.152544453 | 0.334 | 0.329 | 4.73E−35 |
| Hspe1 | 1.98E−39 | −0.157908691 | 0.6 | 0.557 | 4.91E−35 |
| Sqstm1 | 4.03E−39 | 0.16784685 | 0.923 | 0.805 | 1.00E−34 |
| Ndufa7 | 5.40E−39 | −0.18650272 | 0.739 | 0.718 | 1.34E−34 |
| Canx | 1.10E−38 | 0.163661783 | 0.941 | 0.835 | 2.74E−34 |
| Gdi2 | 1.73E−38 | 0.15496797 | 0.782 | 0.605 | 4.30E−34 |
| Sucnr1 | 2.03E−38 | −0.171877738 | 0.101 | 0.201 | 5.04E−34 |
| 1500011K16Rik | 7.21E−38 | −0.156897838 | 0.297 | 0.312 | 1.79E−33 |
| Slc37a4 | 1.47E−37 | 0.154508476 | 0.687 | 0.502 | 3.66E−33 |
| Sfxn1 | 3.26E−37 | 0.177292254 | 0.68 | 0.496 | 8.10E−33 |
| Rplp0 | 2.95E−36 | −0.152097782 | 0.828 | 0.791 | 7.32E−32 |
| Rplp1 | 4.29E−36 | −0.196523046 | 0.782 | 0.757 | 1.06E−31 |
| Ifi27 | 4.78E−36 | 0.228184801 | 0.632 | 0.453 | 1.19E−31 |
| Hsd17b12 | 1.08E−35 | 0.150192807 | 0.811 | 0.647 | 2.69E−31 |
| Gm11273 | 1.47E−35 | −0.154535944 | 0.034 | 0.102 | 3.66E−31 |
| 1100001G20Rik | 1.71E−35 | −0.181522579 | 0.998 | 0.982 | 4.24E−31 |
| Atf4 | 1.03E−34 | 0.170664039 | 0.622 | 0.442 | 2.55E−30 |
| Rps9 | 1.44E−34 | −0.160672445 | 0.733 | 0.675 | 3.57E−30 |
| mt-Ti | 1.42E−33 | −0.175221151 | 0.091 | 0.167 | 3.52E−29 |
| Etnppl | 3.38E−33 | 0.199542145 | 0.769 | 0.606 | 8.40E−29 |
| Krt18 | 3.64E−33 | 0.185376047 | 0.771 | 0.617 | 9.04E−29 |
| Mup20 | 2.28E−32 | −0.244695365 | 1 | 1 | 5.67E−28 |
| Cox5b | 2.57E−32 | −0.184963493 | 0.747 | 0.726 | 6.38E−28 |
| Wdr89 | 4.58E−32 | −0.157854036 | 0.08 | 0.118 | 1.14E−27 |
| Bcar3 | 4.86E−32 | −0.154603512 | 0.132 | 0.193 | 1.21E−27 |
| Lect2 | 1.72E−31 | −0.274252442 | 0.297 | 0.414 | 4.27E−27 |
| Ddt | 1.57E−30 | −0.172944365 | 0.847 | 0.829 | 3.91E−26 |
| Saa3 | 8.77E−29 | 0.298054041 | 0.195 | 0.087 | 2.18E−24 |
| Oat | 5.46E−28 | −0.255537891 | 0.502 | 0.611 | 1.36E−23 |
| Csad | 9.84E−28 | 0.204004276 | 0.872 | 0.744 | 2.44E−23 |
| mmu-mir-6240 | 3.16E−24 | −0.197588471 | 0.054 | 0.115 | 7.85E−20 |
| Cyp7a1 | 2.27E−21 | −0.191761184 | 0.186 | 0.259 | 5.64E−17 |
| Gulo | 1.13E−20 | −0.161523894 | 0.482 | 0.497 | 2.80E−16 |
| Mup7 | 0.007903215 | −0.293978105 | 0.998 | 0.998 | 1 |

TABLE 6
Differentially Expressed genes between time point PH48 and untreated (UT)

| | p_val | avg_logFC | pct. 1 | pct. 2 | p_val_adj |
|---|---|---|---|---|---|
| Saa2 | 0 | 4.43818759 | 0.995 | 0.222 | 0 |
| Saa1 | 0 | 3.650785996 | 0.999 | 0.662 | 0 |
| Orm2 | 0 | 2.405086745 | 0.882 | 0.09 | 0 |
| Lcn2 | 0 | 2.39758181 | 0.862 | 0.07 | 0 |
| Saa3 | 0 | 2.199786317 | 0.629 | 0.087 | 0 |
| Apes | 0 | 2.049485412 | 0.922 | 0.525 | 0 |
| Orm1 | 0 | 1.823760769 | 0.997 | 0.952 | 0 |
| Hp | 0 | 1.584889895 | 1 | 0.999 | 0 |
| Fgl1 | 0 | 1.561880768 | 0.967 | 0.784 | 0 |
| Gm26924 | 0 | 1.445355764 | 1 | 1 | 0 |
| Hpx | 0 | 1.443685235 | 1 | 0.997 | 0 |
| Itih4 | 0 | 1.171742648 | 0.99 | 0.948 | 0 |
| C3 | 0 | 0.982337034 | 0.998 | 0.99 | 0 |
| Apoe | 0 | −0.615882949 | 0.999 | 1 | 0 |
| Apoa2 | 0 | −0.615981195 | 0.997 | 1 | 0 |
| mt-Nd1 | 0 | −1.154630438 | 0.967 | 1 | 0 |
| mt-Nd2 | 0 | −1.189410874 | 0.935 | 0.999 | 0 |
| Mup20 | 0 | −1.237817166 | 0.94 | 1 | 0 |
| mt-Cytb | 0 | −1.378313317 | 0.921 | 0.999 | 0 |
| mt-Co1 | 0 | −1.399110403 | 0.911 | 0.999 | 0 |
| Mup11 | 0 | −1.415092329 | 0.925 | 1 | 0 |
| Mup3 | 0 | −1.423759103 | 0.999 | 1 | 0 |
| mt-Nd4 | 0 | −1.673140231 | 0.851 | 0.998 | 0 |
| Mup4 | 0 | −1.945646142 | 0.168 | 0.898 | 0 |
| Mup9 | 0 | −2.219144293 | 0.238 | 0.976 | 0 |
| Mup17 | 0 | −2.2696211 | 0.683 | 1 | 0 |
| Mup18 | 0 | −2.373348742 | 0.455 | 0.997 | 0 |
| Mup6 | 0 | −2.645373468 | 0.413 | 0.992 | 0 |
| Mup19 | 0 | −3.632170505 | 0.355 | 1 | 0 |
| Car3 | 8.09E−306 | −1.292191881 | 0.738 | 0.974 | 2.01E−301 |
| mt-Nd5 | 5.14E−305 | −1.397897502 | 0.833 | 0.995 | 1.28E−300 |
| Itih3 | 1.63E−301 | 1.223022315 | 0.963 | 0.89 | 4.04E−297 |
| Mt1 | 3.98E−297 | 1.733946488 | 0.899 | 0.556 | 9.88E−293 |
| Mup21 | 5.31E−295 | −1.346645152 | 0.379 | 0.929 | 1.32E−290 |
| n-R5-8s1 | 1.18E−287 | 1.223121237 | 0.464 | 0.031 | 2.94E−283 |
| Mup5 | 8.36E−285 | −1.768987649 | 0.189 | 0.839 | 2.08E−280 |
| 2810007J24Rik | 1.06E−277 | −1.093998287 | 0.598 | 0.943 | 2.63E−273 |
| Mt2 | 8.20E−276 | 1.558205591 | 0.812 | 0.365 | 2.04E−271 |
| Tdo2 | 2.59E−271 | −0.736504592 | 0.9 | 0.991 | 6.44E−267 |
| Rbp4 | 5.33E−264 | −0.580599925 | 0.98 | 0.999 | 1.32E−259 |
| Ankrd55 | 1.01E−251 | −1.950942364 | 0.023 | 0.696 | 2.52E−247 |
| Fga | 3.73E−250 | 0.968748472 | 0.992 | 0.997 | 9.27E−246 |
| Mup12 | 4.44E−248 | −1.210517374 | 0.304 | 0.86 | 1.10E−243 |
| Lars2 | 1.90E−226 | 0.849538102 | 0.996 | 0.994 | 4.73E−222 |
| Gm13775 | 1.60E−220 | −1.145210977 | 0.163 | 0.761 | 3.97E−216 |
| Fabp1 | 3.30E−218 | −0.542167351 | 0.993 | 1 | 8.21E−214 |
| Lrg1 | 6.20E−218 | 1.051046086 | 0.905 | 0.736 | 1.54E−213 |
| Hsd11b1 | 1.59E−213 | −0.998666341 | 0.494 | 0.875 | 3.95E−209 |
| mt-Co2 | 2.22E−213 | −1.417328219 | 0.024 | 0.629 | 5.51E−209 |
| Ces3a | 5.70E−211 | −0.721008165 | 0.796 | 0.967 | 1.41E−206 |
| Hmgcs2 | 2.42E−210 | −0.664269004 | 0.865 | 0.986 | 6.01E−206 |
| Mup15 | 9.41E−207 | −1.364182598 | 0.022 | 0.617 | 2.34E−202 |
| Serpina1d | 2.36E−203 | −0.586852756 | 0.896 | 0.988 | 5.86E−199 |
| Selenbp2 | 3.91E−198 | −0.857199402 | 0.581 | 0.937 | 9.70E−194 |
| Ugt2b5 | 5.94E−195 | −0.683262994 | 0.84 | 0.959 | 1.47E−190 |
| Hrsp12 | 6.38E−187 | −0.800669662 | 0.701 | 0.939 | 1.58E−182 |
| Fgb | 5.69E−184 | 0.677592366 | 0.999 | 1 | 1.41E−179 |
| Gm24601 | 1.08E−180 | 0.675379727 | 0.292 | 0.015 | 2.68E−176 |
| Acaa1b | 3.58E−179 | −0.742478092 | 0.668 | 0.969 | 8.89E−175 |
| mt-Co3 | 3.92E−179 | −1.212388388 | 0.066 | 0.603 | 9.73E−175 |
| Serpina3n | 1.88E−173 | 1.082720183 | 0.805 | 0.574 | 4.67E−169 |
| Fgg | 1.38E−171 | 0.702140119 | 0.996 | 0.991 | 3.42E−167 |
| Ambp | 1.08E−167 | 0.4774812 | 0.995 | 0.993 | 2.69E−163 |
| Ces3b | 2.76E−165 | −0.819558687 | 0.344 | 0.787 | 6.86E−161 |
| Cyp2d9 | 3.51E−155 | −0.616676578 | 0.806 | 0.959 | 8.72E−151 |
| Elovl3 | 7.26E−155 | −0.812250701 | 0.396 | 0.827 | 1.80E−150 |
| Mup16 | 8.52E−154 | −0.87654486 | 0.529 | 0.934 | 2.12E−149 |
| Trf | 6.44E−153 | 0.436833817 | 0.997 | 1 | 1.60E−148 |
| Retsat | 2.20E−152 | −0.784380771 | 0.457 | 0.83 | 5.47E−148 |
| Gm15564 | 1.74E−150 | 0.746211054 | 0.962 | 0.956 | 4.32E−146 |
| Ugt2b36 | 4.45E−150 | −0.629671114 | 0.751 | 0.93 | 1.10E−145 |
| Cox4i1 | 9.84E−146 | −0.57523009 | 0.814 | 0.958 | 2.44E−141 |
| mt-Rnr2 | 1.08E−145 | −0.51967923 | 1 | 1 | 2.69E−141 |
| Rnase4 | 1.72E−145 | 0.836959148 | 0.933 | 0.921 | 4.28E−141 |
| Prtn3 | 3.48E−145 | 0.327267898 | 0.2 | 0.003 | 8.65E−141 |
| Ugt2b1 | 2.29E−144 | −0.722102286 | 0.573 | 0.871 | 5.69E−140 |
| Serpina3k | 1.35E−142 | −0.279762031 | 0.999 | 1 | 3.34E−138 |
| H2-K1 | 7.00E−142 | −0.596584494 | 0.803 | 0.936 | 1.74E−137 |
| Apoa4 | 1.21E−141 | 1.186863065 | 0.658 | 0.339 | 3.01E−137 |
| Cyp2f2 | 2.11E−140 | −0.887034648 | 0.469 | 0.863 | 5.25E−136 |
| Pigr | 4.34E−139 | −0.661452 | 0.741 | 0.947 | 1.08E−134 |
| Rdh7 | 2.25E−136 | −0.496910675 | 0.848 | 0.969 | 5.58E−132 |
| Hsd3b5 | 1.44E−133 | −0.886463454 | 0.146 | 0.587 | 3.58E−129 |
| Mup13 | 7.62E−132 | −0.870972392 | 0.107 | 0.546 | 1.89E−127 |
| Apoa1 | 2.09E−131 | −0.336207866 | 1 | 1 | 5.20E−127 |
| Apoh | 1.60E−130 | −0.417935459 | 0.945 | 0.986 | 3.98E−126 |
| Aadac | 2.72E−126 | −0.747342823 | 0.623 | 0.86 | 6.76E−122 |
| Ephx2 | 6.00E−126 | −0.570800981 | 0.72 | 0.937 | 1.49E−121 |
| Gpx1 | 9.51E−125 | −0.321984135 | 0.991 | 0.999 | 2.36E−120 |
| Gsta3 | 1.01E−122 | −0.579823037 | 0.89 | 0.956 | 2.50E−118 |
| Fam25c | 1.11E−122 | 0.96762143 | 0.653 | 0.356 | 2.76E−118 |
| Alb | 1.03E−121 | −0.427719117 | 1 | 1 | 2.56E−117 |
| mt-Atp6 | 1.35E−121 | −0.914635453 | 0.034 | 0.452 | 3.35E−117 |
| Otc | 6.05E−121 | −0.584972427 | 0.713 | 0.914 | 1.50E−116 |
| Cp | 5.68E−120 | 0.620221202 | 0.952 | 0.943 | 1.41E−115 |
| Dhcr24 | 6.64E−120 | 0.759730578 | 0.862 | 0.783 | 1.65E−115 |
| Apoa5 | 1.27E−119 | −0.470514722 | 0.85 | 0.974 | 3.16E−115 |
| Mup7 | 1.06E−118 | −0.847726732 | 0.936 | 0.998 | 2.63E−114 |
| Cyb5 | 5.01E−113 | −0.486476838 | 0.951 | 0.983 | 1.24E−108 |
| Serpina10 | 1.45E−111 | 0.871044179 | 0.772 | 0.641 | 3.60E−107 |
| Nnmt | 1.55E−111 | 0.768076272 | 0.825 | 0.716 | 3.84E−107 |
| Mug1 | 1.45E−109 | −0.497189268 | 0.891 | 0.989 | 3.61E−105 |
| Slc25a47 | 1.13E−108 | −0.555532536 | 0.691 | 0.927 | 2.81E−104 |
| Acsl1 | 1.46E−108 | −0.568030382 | 0.652 | 0.924 | 3.63E−104 |
| Akr1c6 | 4.20E−108 | −0.501143524 | 0.901 | 0.971 | 1.04E−103 |
| Cfh | 1.01E−107 | 0.588715272 | 0.911 | 0.897 | 2.52E−103 |
| Igfbp2 | 3.61E−107 | −0.576682095 | 0.605 | 0.885 | 8.96E−103 |
| Kng1 | 1.58E−106 | 0.419944891 | 0.979 | 0.991 | 3.92E−102 |
| Cyp4v3 | 5.88E−106 | −0.600614165 | 0.542 | 0.827 | 1.46E−101 |
| Slco1a1 | 2.22E−105 | −0.632477905 | 0.443 | 0.777 | 5.52E−101 |
| Csf2ra | 1.40E−104 | 0.342712543 | 0.158 | 0.005 | 3.48E−100 |
| Mup2 | 8.60E−103 | −0.738721984 | 0.024 | 0.392 | 2.14E−98 |
| Cyp4a10 | 1.05E−102 | −0.698160085 | 0.263 | 0.657 | 2.61E−98 |
| Cfhr2 | 3.06E−102 | −0.630617674 | 0.669 | 0.868 | 7.59E−98 |
| B2m | 8.51E−102 | −0.430851295 | 0.886 | 0.963 | 2.11E−97 |
| mmu-mir-6236 | 2.21E−101 | 0.632369146 | 0.903 | 0.837 | 5.49E−97 |
| D17H6S56E-5 | 8.56E−100 | 0.560241333 | 0.152 | 0.005 | 2.13E−95 |
| Hsd3b7 | 2.07E−97 | −0.581642142 | 0.495 | 0.798 | 5.15E−93 |
| Mup8 | 1.52E−95 | −0.829956435 | 0.025 | 0.373 | 3.77E−91 |
| Khk | 3.58E−95 | −0.530150378 | 0.672 | 0.863 | 8.89E−91 |
| Apoc1 | 4.18E−95 | −0.31073779 | 0.993 | 0.999 | 1.04E−90 |
| Cyp8b1 | 4.37E−95 | −0.565483524 | 0.393 | 0.745 | 1.09E−90 |
| C4bp | 3.00E−94 | 0.530516995 | 0.852 | 0.788 | 7.45E−90 |
| Uox | 5.71E−94 | −0.480422343 | 0.901 | 0.965 | 1.42E−89 |
| Rarres2 | 7.11E−94 | −0.492013911 | 0.712 | 0.905 | 1.76E−89 |
| Serpina12 | 3.03E−93 | −0.612367467 | 0.122 | 0.492 | 7.52E−89 |
| Cxcl14 | 3.17E−93 | 0.268462966 | 0.132 | 0.002 | 7.87E−89 |
| Serpina3c | 3.47E−93 | −0.589926828 | 0.042 | 0.385 | 8.61E−89 |
| Cyp2b10 | 4.76E−92 | −0.995219016 | 0.209 | 0.542 | 1.18E−87 |
| Serpinc1 | 5.56E−92 | −0.333556351 | 0.966 | 0.983 | 1.38E−87 |
| Ugt2a3 | 9.37E−92 | −0.54739922 | 0.46 | 0.773 | 2.33E−87 |
| Serpinf1 | 1.23E−89 | −0.506061854 | 0.631 | 0.864 | 3.06E−85 |
| Hsp90ab1 | 4.96E−88 | 0.455851506 | 0.923 | 0.922 | 1.23E−83 |
| Ttr | 1.56E−86 | −0.286138394 | 0.998 | 1 | 3.87E−82 |
| Adck3 | 2.56E−86 | −0.569692886 | 0.504 | 0.764 | 6.36E−82 |
| Ces1d | 9.77E−86 | −0.445283361 | 0.726 | 0.923 | 2.43E−81 |
| Tmem176a | 1.45E−85 | 0.675060004 | 0.637 | 0.429 | 3.59E−81 |
| C8g | 9.70E−85 | −0.455638561 | 0.751 | 0.91 | 2.41E−80 |
| Isyna1 | 3.42E−84 | 0.320415943 | 0.219 | 0.034 | 8.49E−80 |
| Cd5l | 8.35E−84 | 0.460825248 | 0.199 | 0.026 | 2.07E−79 |
| Cox6c | 1.85E−83 | −0.517417109 | 0.665 | 0.884 | 4.59E−79 |
| Saa4 | 2.78E−83 | 0.635837715 | 0.804 | 0.749 | 6.90E−79 |
| Ccnd1 | 2.52E−82 | 0.385849227 | 0.218 | 0.035 | 6.27E−78 |
| Cyp2c54 | 9.56E−82 | −0.542960224 | 0.426 | 0.759 | 2.37E−77 |
| Serpina1a | 1.36E−79 | −0.35536875 | 0.932 | 0.991 | 3.38E−75 |
| P4hb | 6.98E−79 | 0.38506968 | 0.949 | 0.941 | 1.73E−74 |
| Serpina1b | 8.00E−79 | −0.294480791 | 0.96 | 0.996 | 1.99E−74 |
| Tmem176b | 1.62E−78 | 0.612457008 | 0.744 | 0.632 | 4.03E−74 |
| Gas6 | 2.14E−78 | 0.35878079 | 0.215 | 0.036 | 5.31E−74 |
| Gm23935 | 8.90E−78 | 0.562414557 | 0.975 | 0.925 | 2.21E−73 |
| Ahcy | 3.00E−77 | −0.455985571 | 0.701 | 0.916 | 7.46E−73 |
| mt-Nd6 | 4.60E−77 | −0.560268731 | 0.223 | 0.577 | 1.14E−72 |
| Hmgcs1 | 1.63E−76 | 0.702526059 | 0.819 | 0.703 | 4.05E−72 |
| Serpina1e | 4.63E−76 | −0.308360801 | 0.99 | 0.999 | 1.15E−71 |
| Etnppl | 1.08E−75 | −0.549126978 | 0.257 | 0.606 | 2.68E−71 |
| Aug | 1.14E−75 | 0.644516227 | 0.773 | 0.687 | 2.84E−71 |
| Marco | 4.70E−75 | 0.30539135 | 0.106 | 0.002 | 1.17E−70 |
| 1500017E21Rik | 7.78E−74 | −0.517930222 | 0.164 | 0.476 | 1.93E−69 |
| Ahsg | 2.93E−73 | 0.281450293 | 0.998 | 1 | 7.29E−69 |
| Atp5f1 | 4.47E−73 | −0.460709866 | 0.681 | 0.869 | 1.11E−68 |
| Ndufb6 | 1.65E−72 | −0.479162745 | 0.49 | 0.759 | 4.11E−68 |
| Ces1f | 3.89E−71 | −0.45903711 | 0.577 | 0.817 | 9.66E−67 |
| Cebpa | 1.18E−70 | −0.481611184 | 0.37 | 0.672 | 2.92E−66 |
| Cyp4a12a | 4.03E−70 | −0.4760363 | 0.275 | 0.597 | 1.00E−65 |
| Baat | 4.05E−70 | −0.445833101 | 0.496 | 0.767 | 1.01E−65 |
| Inmt | 6.48E−70 | −0.502414474 | 0.676 | 0.884 | 1.61E−65 |
| Gm12744 | 6.80E−70 | 0.350643059 | 0.231 | 0.05 | 1.69E−65 |
| Pon1 | 7.92E−69 | −0.42825679 | 0.753 | 0.906 | 1.97E−64 |
| Aldob | 9.05E−69 | −0.254127817 | 0.986 | 0.996 | 2.25E−64 |
| Clu | 1.45E−68 | 0.374886754 | 0.954 | 0.958 | 3.60E−64 |
| Rbp1 | 2.09E−68 | 0.396801127 | 0.264 | 0.069 | 5.19E−64 |
| Fabp2 | 2.20E−68 | −0.488533591 | 0.305 | 0.622 | 5.45E−64 |
| Serpina1c | 4.97E−68 | −0.279835146 | 0.948 | 0.998 | 1.24E−63 |
| Sod1 | 6.75E−68 | −0.277853669 | 0.952 | 0.983 | 1.68E−63 |
| Cdo1 | 8.26E−68 | −0.343333854 | 0.843 | 0.96 | 2.05E−63 |
| Steap4 | 1.90E−67 | 0.631013696 | 0.455 | 0.222 | 4.71E−63 |
| Cml1 | 2.23E−67 | −0.47269345 | 0.507 | 0.757 | 5.55E−63 |
| Ech1 | 1.04E−66 | −0.414674796 | 0.617 | 0.856 | 2.57E−62 |
| Insig2 | 1.92E−66 | −0.447606183 | 0.554 | 0.81 | 4.77E−62 |
| Cox6a1 | 6.74E−66 | −0.390617907 | 0.665 | 0.887 | 1.67E−61 |
| Azgp1 | 4.66E−65 | −0.286918536 | 0.904 | 0.968 | 1.16E−60 |
| Ces1c | 2.36E−64 | −0.274525359 | 0.96 | 0.985 | 5.85E−60 |
| Pah | 3.48E−64 | −0.379853874 | 0.71 | 0.891 | 8.65E−60 |
| C1qb | 1.01E−63 | 0.361309767 | 0.163 | 0.024 | 2.50E−59 |
| Lipc | 1.28E−63 | −0.423055461 | 0.294 | 0.59 | 3.19E−59 |
| Dio1 | 3.68E−63 | −0.469732353 | 0.255 | 0.542 | 9.15E−59 |
| Aox3 | 4.95E−63 | −0.429460462 | 0.705 | 0.894 | 1.23E−58 |
| Gstm1 | 1.23E−62 | −0.448355325 | 0.783 | 0.935 | 3.06E−58 |
| Mgst1 | 1.50E−62 | −0.286240934 | 0.997 | 0.997 | 3.71E−58 |
| Rnf125 | 4.04E−62 | −0.423361675 | 0.201 | 0.493 | 1.00E−57 |
| Slc27a5 | 4.35E−62 | −0.374625102 | 0.749 | 0.893 | 1.08E−57 |
| Serpina3m | 5.13E−62 | 0.519751144 | 0.76 | 0.722 | 1.28E−57 |
| Amy1 | 5.64E−62 | −0.441174 | 0.383 | 0.676 | 1.40E−57 |
| Mki67 | 6.42E−62 | 0.39305852 | 0.086 | 0.001 | 1.59E−57 |
| Tmem205 | 2.42E−61 | −0.363762607 | 0.791 | 0.934 | 6.01E−57 |
| Cyp2a12 | 2.80E−61 | −0.396631721 | 0.664 | 0.884 | 6.96E−57 |
| Acnat2 | 7.56E−61 | 0.33547101 | 0.252 | 0.069 | 1.88E−56 |
| Angptl3 | 6.16E−60 | −0.315601765 | 0.879 | 0.944 | 1.53E−55 |
| Qsox1 | 9.01E−60 | 0.551776463 | 0.752 | 0.707 | 2.24E−55 |
| Top2a | 4.44E−59 | 0.33486876 | 0.09 | 0.003 | 1.10E−54 |
| Cyp7b1 | 5.53E−59 | −0.438158056 | 0.496 | 0.757 | 1.37E−54 |
| Hpd | 8.05E−59 | −0.317943335 | 0.91 | 0.976 | 2.00E−54 |
| Cyp3a25 | 9.34E−59 | −0.34306526 | 0.745 | 0.919 | 2.32E−54 |
| Ugp2 | 2.65E−58 | −0.394209561 | 0.486 | 0.749 | 6.59E−54 |
| Clpx | 4.49E−58 | −0.410840842 | 0.571 | 0.783 | 1.11E−53 |
| Clec4f | 4.74E−58 | 0.380337956 | 0.2 | 0.043 | 1.18E−53 |
| Ndufa4 | 4.11E−57 | −0.4035957 | 0.71 | 0.889 | 1.02E−52 |
| Gm20594 | 4.89E−57 | −0.573781187 | 0.048 | 0.294 | 1.21E−52 |
| Cyp2c50 | 6.18E−57 | −0.467298435 | 0.656 | 0.888 | 1.54E−52 |
| Pzp | 1.05E−56 | 0.382796555 | 0.975 | 0.986 | 2.61E−52 |
| Nudt7 | 3.82E−56 | −0.566043759 | 0.662 | 0.83 | 9.48E−52 |
| Uqcrq | 4.46E−56 | −0.341869458 | 0.857 | 0.951 | 1.11E−51 |
| Oaz1 | 5.13E−56 | −0.426286153 | 0.499 | 0.75 | 1.27E−51 |
| Rpl41 | 6.29E−56 | 0.444557582 | 0.528 | 0.278 | 1.56E−51 |
| Cdca3 | 7.55E−56 | 0.286393603 | 0.1 | 0.007 | 1.87E−51 |
| Slc10a1 | 3.28E−55 | −0.395488081 | 0.688 | 0.843 | 8.15E−51 |
| Ifitm2 | 4.32E−55 | 0.526891524 | 0.532 | 0.347 | 1.07E−50 |
| Scp2 | 4.62E−55 | −0.319519472 | 0.902 | 0.986 | 1.15E−50 |
| Rps27l | 3.21E−54 | −0.377566144 | 0.632 | 0.849 | 7.98E−50 |
| Sco2 | 4.20E−54 | −0.395951512 | 0.24 | 0.508 | 1.04E−49 |
| Esd | 7.80E−54 | −0.381539462 | 0.549 | 0.784 | 1.94E−49 |
| Cyp2d10 | 1.51E−53 | −0.325332653 | 0.742 | 0.921 | 3.76E−49 |
| Ndufb11 | 2.89E−53 | −0.398618793 | 0.471 | 0.735 | 7.18E−49 |
| Chchd2 | 6.84E−53 | −0.369755887 | 0.769 | 0.916 | 1.70E−48 |
| Idh3b | 1.69E−52 | −0.360516268 | 0.387 | 0.655 | 4.19E−48 |
| Dbi | 3.82E−52 | 0.360631658 | 0.969 | 0.971 | 9.49E−48 |
| Bckdha | 9.15E−52 | −0.404702898 | 0.365 | 0.63 | 2.27E−47 |
| Glo1 | 1.41E−51 | −0.379233492 | 0.498 | 0.749 | 3.51E−47 |
| Glud1 | 1.89E−51 | 0.359099708 | 0.901 | 0.905 | 4.69E−47 |
| M2 | 5.51E−51 | −0.333366521 | 0.297 | 0.579 | 1.37E−46 |
| Sdhb | 1.01E−50 | −0.357362255 | 0.577 | 0.797 | 2.51E−46 |
| Cat | 4.03E−50 | −0.286016441 | 0.912 | 0.971 | 1.00E−45 |
| Ces1e | 8.30E−50 | −0.356936678 | 0.304 | 0.576 | 2.06E−45 |
| Tat | 9.87E−50 | 0.369618242 | 0.91 | 0.903 | 2.45E−45 |
| Cox5b | 1.37E−49 | −0.393691854 | 0.454 | 0.726 | 3.39E−45 |
| Mup10 | 1.94E−49 | 0.281431551 | 0.781 | 0.692 | 4.82E−45 |
| Aqp8 | 3.30E−49 | −0.359788885 | 0.242 | 0.505 | 8.19E−45 |
| Atp5g3 | 9.30E−49 | −0.302277876 | 0.74 | 0.911 | 2.31E−44 |
| Hint2 | 1.05E−48 | −0.373773572 | 0.433 | 0.676 | 2.60E−44 |
| Actb | 1.06E−48 | 0.399035393 | 0.897 | 0.886 | 2.62E−44 |
| Itm2b | 1.69E−48 | −0.261048932 | 0.869 | 0.951 | 4.20E−44 |
| Cox7b | 4.24E−48 | −0.386524219 | 0.704 | 0.878 | 1.05E−43 |
| Fmo5 | 7.05E−48 | 0.379271143 | 0.841 | 0.837 | 1.75E−43 |
| Etfdh | 7.29E−48 | −0.33708519 | 0.436 | 0.7 | 1.81E−43 |
| 1100001G20Rik | 1.66E−47 | 0.310291029 | 0.982 | 0.982 | 4.13E−43 |
| Gsto1 | 2.07E−47 | −0.368935961 | 0.269 | 0.52 | 5.15E−43 |
| Sds | 3.22E−47 | −0.352238286 | 0.35 | 0.67 | 7.99E−43 |
| Hsd3b3 | 1.54E−46 | −0.347460551 | 0.359 | 0.605 | 3.82E−42 |
| Slc27a2 | 8.99E−46 | −0.305294922 | 0.812 | 0.949 | 2.23E−41 |
| Hsd17b6 | 1.04E−45 | −0.36218325 | 0.189 | 0.453 | 2.58E−41 |
| Gm4076 | 1.16E−45 | −0.345826319 | 0.001 | 0.184 | 2.87E−41 |
| Mat1a | 2.28E−45 | 0.378728792 | 0.938 | 0.966 | 5.65E−41 |
| Abcd3 | 5.85E−45 | −0.326838469 | 0.677 | 0.879 | 1.45E−40 |
| Cyp2c37 | 6.44E−45 | −0.412938667 | 0.326 | 0.608 | 1.60E−40 |
| Mdh1 | 8.20E−45 | −0.329025797 | 0.747 | 0.887 | 2.04E−40 |
| Prdx4 | 1.37E−44 | −0.378024564 | 0.368 | 0.607 | 3.39E−40 |
| Hsd17b13 | 2.14E−44 | −0.376251609 | 0.464 | 0.74 | 5.32E−40 |
| Proc | 6.67E−44 | −0.319776879 | 0.696 | 0.874 | 1.66E−39 |
| Agxt | 7.21E−44 | −0.303827932 | 0.555 | 0.789 | 1.79E−39 |
| Gls2 | 8.71E−44 | −0.353616749 | 0.395 | 0.657 | 2.16E−39 |
| Cpb2 | 4.37E−43 | 0.395699062 | 0.838 | 0.813 | 1.09E−38 |
| Ugt2b38 | 4.82E−43 | −0.250921219 | 0.021 | 0.209 | 1.20E−38 |
| Cyp2a5 | 5.97E−43 | −0.525680085 | 0.295 | 0.555 | 1.48E−38 |
| Ndufb9 | 8.65E−43 | −0.291910914 | 0.729 | 0.888 | 2.15E−38 |
| Cdc20 | 1.26E−42 | 0.272554242 | 0.062 | 0.001 | 3.12E−38 |
| Rabac1 | 1.31E−42 | −0.342063679 | 0.284 | 0.524 | 3.25E−38 |
| Slc22a30 | 3.13E−42 | −0.331870745 | 0.26 | 0.493 | 7.77E−38 |
| Etfa | 3.25E−42 | −0.33793403 | 0.595 | 0.802 | 8.07E−38 |
| Cd302 | 7.31E−42 | 0.384976006 | 0.864 | 0.86 | 1.82E−37 |
| Csf1r | 1.53E−41 | 0.284684372 | 0.106 | 0.015 | 3.79E−37 |
| Cth | 1.76E−41 | −0.332234114 | 0.681 | 0.838 | 4.36E−37 |
| Atp5j | 2.00E−41 | −0.302644835 | 0.512 | 0.757 | 4.96E−37 |
| Uqcrfs1 | 2.26E−41 | −0.335765197 | 0.583 | 0.787 | 5.60E−37 |
| Cyp27a1 | 5.89E−41 | −0.326228094 | 0.636 | 0.837 | 1.46E−36 |
| Hal | 1.22E−40 | −0.304647432 | 0.47 | 0.749 | 3.02E−36 |
| Ppp2r5a | 1.36E−40 | −0.315098874 | 0.366 | 0.594 | 3.37E−36 |
| Sar1b | 1.79E−40 | −0.301891484 | 0.56 | 0.772 | 4.44E−36 |
| Aldh6a1 | 1.87E−40 | −0.30870904 | 0.471 | 0.704 | 4.64E−36 |
| Il1rap | 7.80E−40 | −0.302807638 | 0.35 | 0.58 | 1.94E−35 |
| S100a1 | 8.91E−40 | −0.355544479 | 0.454 | 0.661 | 2.21E−35 |
| Creg1 | 1.17E−39 | −0.258963412 | 0.787 | 0.926 | 2.90E−35 |
| Gstm2 | 1.61E−39 | 0.466218536 | 0.345 | 0.169 | 3.99E−35 |
| Mbl2 | 2.21E−39 | −0.254854549 | 0.625 | 0.829 | 5.49E−35 |
| Apoc2 | 2.61E−39 | 0.517142972 | 0.803 | 0.802 | 6.48E−35 |
| 1300017J02Rik | 4.81E−39 | −0.308343818 | 0.59 | 0.803 | 1.20E−34 |
| Acat1 | 7.30E−39 | −0.290805818 | 0.628 | 0.838 | 1.81E−34 |
| Pklr | 2.19E−38 | −0.283149611 | 0.157 | 0.368 | 5.43E−34 |
| Krt18 | 2.37E−38 | 0.520354076 | 0.681 | 0.617 | 5.88E−34 |
| Dpys | 3.56E−38 | −0.313405671 | 0.447 | 0.682 | 8.84E−34 |
| Aadat | 3.68E−38 | −0.325731706 | 0.28 | 0.507 | 9.13E−34 |
| Mup1 | 4.45E−38 | −0.62326727 | 0.367 | 0.583 | 1.11E−33 |
| Gm5096 | 5.11E−38 | −0.336704156 | 0.249 | 0.468 | 1.27E−33 |
| Cd81 | 6.03E−38 | −0.30883572 | 0.435 | 0.675 | 1.50E−33 |
| Ndufc2 | 6.26E−38 | −0.370889898 | 0.371 | 0.579 | 1.56E−33 |
| Bphl | 6.83E−38 | −0.299478155 | 0.355 | 0.579 | 1.70E−33 |
| Qdpr | 7.25E−38 | −0.316595182 | 0.649 | 0.814 | 1.80E−33 |
| Gsta2 | 1.84E−37 | −0.284180552 | 0.129 | 0.333 | 4.56E−33 |
| Acsm3 | 2.12E−37 | −0.280708005 | 0.191 | 0.407 | 5.27E−33 |
| Cyp2d26 | 8.21E−37 | −0.270278674 | 0.755 | 0.903 | 2.04E−32 |
| Cyp2c44 | 8.41E−37 | −0.312764543 | 0.48 | 0.698 | 2.09E−32 |
| Hagh | 1.20E−36 | −0.288430674 | 0.699 | 0.858 | 2.99E−32 |
| Ndufa6 | 1.25E−36 | −0.330965466 | 0.603 | 0.798 | 3.11E−32 |
| Ces2a | 1.73E−36 | −0.288140076 | 0.628 | 0.846 | 4.30E−32 |
| Fn1 | 2.06E−36 | 0.469732274 | 0.819 | 0.821 | 5.12E−32 |
| Gm10250 | 2.24E−36 | −0.304478582 | 0.068 | 0.251 | 5.57E−32 |
| Gas5 | 2.54E−36 | −0.298417518 | 0.294 | 0.527 | 6.31E−32 |
| Alas1 | 3.08E−36 | 0.423999349 | 0.749 | 0.729 | 7.65E−32 |
| Hebp1 | 3.34E−36 | −0.297464234 | 0.539 | 0.75 | 8.29E−32 |
| Uqcrb | 5.39E−36 | −0.339504918 | 0.425 | 0.641 | 1.34E−31 |
| Gm4788 | 6.02E−36 | −0.297042332 | 0.575 | 0.766 | 1.49E−31 |
| Slc2a2 | 1.00E−35 | −0.291267847 | 0.261 | 0.479 | 2.49E−31 |
| Tmsb4x | 3.03E−35 | 0.347657914 | 0.183 | 0.057 | 7.53E−31 |
| Cela1 | 4.17E−35 | −0.252611197 | 0.112 | 0.304 | 1.03E−30 |
| Hgd | 9.92E−35 | −0.273998205 | 0.693 | 0.861 | 2.46E−30 |
| Dnajc12 | 1.02E−34 | 0.312907586 | 0.256 | 0.108 | 2.54E−30 |
| Gadd45g | 1.32E−34 | −0.336240737 | 0.402 | 0.639 | 3.29E−30 |
| 1110008F13Rik | 1.52E−34 | −0.284458511 | 0.424 | 0.648 | 3.77E−30 |
| Csad | 1.75E−34 | −0.308703065 | 0.516 | 0.744 | 4.35E−30 |
| Gabarapl1 | 8.13E−34 | −0.297156374 | 0.563 | 0.779 | 2.02E−29 |
| Eci1 | 1.80E−33 | −0.292606814 | 0.482 | 0.709 | 4.47E−29 |
| Sparcl1 | 2.40E−33 | −0.253616986 | 0 | 0.136 | 5.97E−29 |
| Atp5h | 2.89E−33 | −0.352362982 | 0.47 | 0.682 | 7.17E−29 |
| Amdhd1 | 3.26E−33 | −0.272469871 | 0.442 | 0.661 | 8.10E−29 |
| Lyz2 | 4.87E−33 | 0.275390862 | 0.132 | 0.032 | 1.21E−28 |
| Uqcrh | 6.10E−33 | −0.290259964 | 0.629 | 0.81 | 1.52E−28 |
| 0610011F06Rik | 1.41E−32 | −0.28593883 | 0.437 | 0.653 | 3.51E−28 |
| Ftcd | 2.20E−32 | −0.299187456 | 0.473 | 0.691 | 5.47E−28 |
| Cyp4f14 | 2.60E−32 | −0.283565035 | 0.394 | 0.6 | 6.47E−28 |
| BC048546 | 4.68E−32 | 0.287060939 | 0.265 | 0.119 | 1.16E−27 |
| Ttc39c | 9.43E−32 | −0.289612311 | 0.454 | 0.642 | 2.34E−27 |
| Mup14 | 9.56E−32 | −0.497427273 | 0.421 | 0.616 | 2.38E−27 |
| Ndufv1 | 9.79E−32 | −0.256865664 | 0.462 | 0.683 | 2.43E−27 |
| Pdhb | 2.21E−31 | −0.271619421 | 0.269 | 0.468 | 5.48E−27 |
| Hsd17b11 | 3.37E−31 | −0.272884574 | 0.379 | 0.592 | 8.36E−27 |
| Gstm3 | 5.90E−31 | 0.793361403 | 0.543 | 0.414 | 1.46E−26 |
| Fcgrt | 7.31E−31 | −0.270945405 | 0.382 | 0.599 | 1.81E−26 |
| Arl4d | 1.23E−30 | −0.288941612 | 0.245 | 0.437 | 3.05E−26 |
| Lyrm5 | 1.24E−30 | −0.251171881 | 0.206 | 0.396 | 3.08E−26 |
| Bnip3 | 1.25E−30 | −0.275946132 | 0.335 | 0.55 | 3.10E−26 |
| Pygl | 5.20E−30 | −0.25405228 | 0.572 | 0.776 | 1.29E−25 |
| C8b | 6.04E−30 | −0.251084368 | 0.722 | 0.881 | 1.50E−25 |
| Atp5j2 | 1.03E−29 | −0.255051714 | 0.678 | 0.852 | 2.55E−25 |
| Igfbp1 | 1.08E−29 | 0.466237818 | 0.716 | 0.717 | 2.67E−25 |
| Fxyd1 | 1.19E−29 | −0.273422293 | 0.499 | 0.721 | 2.95E−25 |
| Slc41a2 | 2.25E−29 | 0.318516467 | 0.278 | 0.134 | 5.60E−25 |
| Nit2 | 2.74E−29 | −0.255321274 | 0.375 | 0.577 | 6.79E−25 |
| Sdha | 3.20E−29 | −0.286156431 | 0.554 | 0.747 | 7.96E−25 |
| Pccb | 3.22E−29 | −0.266663529 | 0.433 | 0.644 | 8.00E−25 |
| Park7 | 3.63E−29 | −0.270929309 | 0.572 | 0.784 | 9.02E−25 |
| Cmbl | 3.89E−29 | −0.265365994 | 0.476 | 0.678 | 9.66E−25 |
| Upp2 | 4.15E−29 | −0.265836776 | 0.451 | 0.673 | 1.03E−24 |
| Prg4 | 5.85E−29 | 0.355545985 | 0.344 | 0.188 | 1.45E−24 |
| Itih2 | 9.24E−29 | 0.312933325 | 0.866 | 0.884 | 2.29E−24 |
| Zfp683 | 2.98E−28 | −0.421689112 | 0 | 0.116 | 7.40E−24 |
| Ces2e | 3.51E−28 | −0.272708804 | 0.335 | 0.535 | 8.71E−24 |
| Cebpb | 6.97E−28 | −0.306330499 | 0.272 | 0.45 | 1.73E−23 |
| Pbld1 | 8.97E−28 | −0.264627734 | 0.366 | 0.565 | 2.23E−23 |
| Gm9843 | 3.14E−27 | −0.298882573 | 0.135 | 0.299 | 7.79E−23 |
| Adipor2 | 4.45E−27 | −0.252551165 | 0.638 | 0.807 | 1.11E−22 |
| Ephx1 | 5.06E−27 | −0.261640077 | 0.589 | 0.8 | 1.26E−22 |
| Pim3 | 1.08E−26 | −0.255337561 | 0.345 | 0.537 | 2.68E−22 |
| C6 | 8.21E−26 | −0.255102014 | 0.434 | 0.642 | 2.04E−21 |
| Hspa8 | 1.73E−25 | 0.302152434 | 0.744 | 0.756 | 4.31E−21 |
| Rps2 | 8.86E−25 | −0.269321392 | 0.544 | 0.757 | 2.20E−20 |
| Ly6e | 2.02E−24 | 0.525599776 | 0.49 | 0.386 | 5.02E−20 |
| Txnip | 5.00E−24 | −0.310098222 | 0.142 | 0.297 | 1.24E−19 |
| Dynll1 | 7.21E−24 | 0.289016997 | 0.283 | 0.154 | 1.79E−19 |
| Cyp2e1 | 9.88E−24 | −0.298608744 | 0.715 | 0.911 | 2.45E−19 |
| Tspan4 | 1.37E−23 | 0.32084381 | 0.364 | 0.23 | 3.41E−19 |
| Thrsp | 6.26E−23 | −0.374383397 | 0.497 | 0.627 | 1.55E−18 |
| Il1r1 | 1.12E−22 | 0.299449962 | 0.301 | 0.167 | 2.79E−18 |
| Kng2 | 1.19E−22 | 0.3169695 | 0.687 | 0.684 | 2.95E−18 |
| 2200002D01Rik | 6.87E−22 | 0.257614019 | 0.261 | 0.136 | 1.71E−17 |
| G6pc | 1.49E−21 | −0.259264856 | 0.597 | 0.752 | 3.71E−17 |
| Cyp51 | 1.75E−21 | 0.40339747 | 0.67 | 0.649 | 4.34E−17 |
| Cyp7a1 | 2.38E−21 | 0.598824806 | 0.372 | 0.259 | 5.91E−17 |
| Tgm2 | 2.39E−20 | 0.37123494 | 0.374 | 0.258 | 5.94E−16 |
| Mlec | 5.88E−20 | 0.400321297 | 0.436 | 0.323 | 1.46E−15 |
| Myh9 | 6.40E−20 | 0.438269269 | 0.426 | 0.318 | 1.59E−15 |
| Smc4 | 1.53E−18 | 0.338338192 | 0.177 | 0.083 | 3.80E−14 |
| Rn7sk | 2.49E−18 | −0.276399379 | 0.073 | 0.186 | 6.19E−14 |
| Hrg | 2.76E−18 | 0.270451116 | 0.807 | 0.831 | 6.86E−14 |
| Snx10 | 5.15E−18 | 0.253822304 | 0.304 | 0.188 | 1.28E−13 |
| Gm24245 | 1.34E−17 | −0.264578368 | 0.097 | 0.214 | 3.33E−13 |
| Eef1a1 | 5.03E−17 | 0.254969818 | 0.794 | 0.85 | 1.25E−12 |
| Sdc1 | 5.77E−17 | 0.330139692 | 0.531 | 0.469 | 1.43E−12 |
| Sult1a1 | 8.71E−17 | 0.344836675 | 0.69 | 0.707 | 2.16E−12 |
| Ctsb | 8.73E−17 | 0.310468937 | 0.777 | 0.781 | 2.17E−12 |
| Hspa5 | 9.12E−17 | 0.270882455 | 0.929 | 0.944 | 2.27E−12 |
| Gjb2 | 9.83E−17 | 0.328679811 | 0.61 | 0.572 | 2.44E−12 |
| Dap | 1.60E−16 | 0.290008119 | 0.667 | 0.654 | 3.97E−12 |
| Slc3a1 | 7.18E−16 | 0.321802388 | 0.29 | 0.189 | 1.78E−11 |
| Hsph1 | 4.12E−15 | 0.345997935 | 0.353 | 0.255 | 1.02E−10 |
| Mrap | 4.18E−15 | 0.280974611 | 0.376 | 0.272 | 1.04E−10 |
| Grn | 1.80E−14 | 0.293077345 | 0.48 | 0.396 | 4.47E−10 |
| Lpgat1 | 4.23E−14 | 0.347767347 | 0.545 | 0.502 | 1.05E−09 |
| Creld2 | 4.49E−13 | 0.362477611 | 0.385 | 0.296 | 1.11E−08 |
| Cpox | 1.20E−12 | 0.294319953 | 0.513 | 0.45 | 2.98E−08 |
| Ftl1 | 6.02E−12 | −0.259151442 | 0.298 | 0.409 | 1.49E−07 |
| Ebp | 1.48E−11 | 0.252819864 | 0.634 | 0.666 | 3.69E−07 |
| Fdps | 1.57E−10 | 0.303232732 | 0.492 | 0.445 | 3.91E−06 |
| Btg2 | 9.56E−10 | 0.280923811 | 0.321 | 0.241 | 2.37E−05 |
| Gm26917 | 1.01E−09 | 0.475554164 | 0.255 | 0.181 | 2.52E−05 |
| Lrp1 | 1.11E−09 | 0.314138344 | 0.597 | 0.591 | 2.75E−05 |
| Stat3 | 1.14E−09 | 0.308012615 | 0.386 | 0.318 | 2.82E−05 |
| Ugdh | 1.89E−09 | 0.260443532 | 0.602 | 0.604 | 4.68E−05 |
| Tmpo | 2.13E−09 | 0.2573532 | 0.236 | 0.162 | 5.30E−05 |
| Cxcl1 | 2.93E−09 | 0.353012683 | 0.374 | 0.314 | 7.28E−05 |
| Lss | 5.24E−09 | 0.294433908 | 0.282 | 0.211 | 0.000130068 |
| Hyou1 | 6.67E−09 | 0.315760783 | 0.44 | 0.385 | 0.000165574 |
| Akr1d1 | 1.25E−08 | 0.258293189 | 0.537 | 0.515 | 0.00030931 |
| Snord13 | 1.32E−08 | 0.408788625 | 0.247 | 0.175 | 0.000327047 |
| Acsl4 | 1.24E−07 | 0.273275871 | 0.376 | 0.323 | 0.003086449 |
| Lsr | 2.32E−07 | 0.262580416 | 0.512 | 0.517 | 0.005770468 |
| Tuba1c | 2.44E−07 | 0.302939755 | 0.415 | 0.37 | 0.006056912 |
| Caprin1 | 6.74E−07 | 0.303020851 | 0.497 | 0.481 | 0.016747307 |
| Atl3 | 8.51E−07 | 0.255322039 | 0.334 | 0.27 | 0.021123411 |
| Manf | 9.37E−07 | 0.282950631 | 0.466 | 0.44 | 0.023268676 |
| Txnrd1 | 1.31E−06 | 0.252234489 | 0.468 | 0.441 | 0.032529357 |
| Avpi1 | 4.09E−06 | 0.30186919 | 0.409 | 0.377 | 0.101466193 |
| Krt8 | 7.36E−06 | 0.252276759 | 0.552 | 0.578 | 0.182829207 |
| Sqle | 5.87E−05 | 0.27714068 | 0.379 | 0.349 | 1 |
| Nucks1 | 9.19E−05 | 0.271055696 | 0.445 | 0.43 | 1 |
| Lect2 | 0.000105656 | 0.288512824 | 0.427 | 0.414 | 1 |
| Oat | 0.000221224 | 0.741679089 | 0.542 | 0.611 | 1 |
| Tubb5 | 0.00072943 | 0.313824522 | 0.304 | 0.271 | 1 |
| Tubb4b | 0.032455023 | 0.28149027 | 0.347 | 0.343 | 1 |
| Glul | 0.033844352 | 0.611936929 | 0.203 | 0.18 | 1 |

TABLE 7
Differentially Expressed genes between timepoint PH120 and Untreated (UT)

| | p_val | avg_logFC | pct. 1 | pct. 2 | p_val_adj |
|---|---|---|---|---|---|
| Saa2 | 0 | 3.598008691 | 0.948 | 0.222 | 0 |
| Saa1 | 0 | 2.959370227 | 0.996 | 0.662 | 0 |
| Lcn2 | 0 | 2.127649068 | 0.925 | 0.07 | 0 |
| Mt1 | 0 | 1.65972779 | 0.975 | 0.556 | 0 |
| Orm2 | 0 | 1.536747401 | 0.813 | 0.09 | 0 |
| Gm15564 | 0 | 1.485885928 | 1 | 0.956 | 0 |
| Mt2 | 0 | 1.471840722 | 0.93 | 0.365 | 0 |
| Lars2 | 0 | 1.419129056 | 1 | 0.994 | 0 |
| Fgl1 | 0 | 1.379926505 | 0.993 | 0.784 | 0 |
| Gm26924 | 0 | 1.379756734 | 1 | 1 | 0 |
| Lrg1 | 0 | 1.260509735 | 0.982 | 0.736 | 0 |
| Serpina3n | 0 | 1.205364333 | 0.948 | 0.574 | 0 |
| Itih4 | 0 | 1.18011817 | 0.998 | 0.948 | 0 |
| Itih3 | 0 | 1.177912468 | 0.991 | 0.89 | 0 |
| Saa4 | 0 | 1.158596154 | 0.982 | 0.749 | 0 |
| Hp | 0 | 1.107075607 | 0.999 | 0.999 | 0 |
| Ly6e | 0 | 1.093957206 | 0.821 | 0.386 | 0 |
| C3 | 0 | 1.071950932 | 0.999 | 0.99 | 0 |
| n-R5-8s1 | 0 | 1.066654791 | 0.578 | 0.031 | 0 |
| Orm1 | 0 | 1.05827481 | 1 | 0.952 | 0 |
| Fga | 0 | 1.039432587 | 0.999 | 0.997 | 0 |
| Rnase4 | 0 | 1.031156304 | 0.993 | 0.921 | 0 |
| Fn1 | 0 | 1.018772906 | 0.968 | 0.821 | 0 |
| Hpx | 0 | 0.994363825 | 1 | 0.997 | 0 |
| Cfh | 0 | 0.83288399 | 0.991 | 0.897 | 0 |
| C4b | 0 | 0.820022988 | 0.98 | 0.883 | 0 |
| Clu | 0 | 0.745288027 | 0.997 | 0.958 | 0 |
| Fgg | 0 | 0.686743278 | 1 | 0.991 | 0 |
| Gm24601 | 0 | 0.675086715 | 0.422 | 0.015 | 0 |
| Pzp | 0 | 0.636851805 | 0.999 | 0.986 | 0 |
| Fgb | 0 | 0.605783409 | 0.999 | 1 | 0 |
| Plg | 0 | 0.596524395 | 0.997 | 0.961 | 0 |
| Kng1 | 0 | 0.546835494 | 0.999 | 0.991 | 0 |
| Serpina1e | 0 | −0.72519719 | 0.993 | 0.999 | 0 |
| Cyp3a11 | 0 | −0.803553063 | 0.997 | 1 | 0 |
| Fabp1 | 0 | −0.826491833 | 0.995 | 1 | 0 |
| mt-Rnr1 | 0 | −0.844781496 | 1 | 1 | 0 |
| Acaa1b | 0 | −0.855527427 | 0.792 | 0.969 | 0 |
| mt-Rnr2 | 0 | −0.967453486 | 1 | 1 | 0 |
| Mup20 | 0 | −1.081531043 | 0.99 | 1 | 0 |
| Car3 | 0 | −1.168630683 | 0.905 | 0.974 | 0 |
| Ces1d | 0 | −1.201503329 | 0.586 | 0.923 | 0 |
| Mup12 | 0 | −1.228078411 | 0.39 | 0.86 | 0 |
| Mup16 | 0 | −1.245137265 | 0.578 | 0.934 | 0 |
| Mup3 | 0 | −1.310718039 | 1 | 1 | 0 |
| mt-Nd2 | 0 | −1.350784061 | 0.975 | 0.999 | 0 |
| mt-Nd5 | 0 | −1.49645814 | 0.9 | 0.995 | 0 |
| mt-Nd1 | 0 | −1.543996447 | 0.98 | 1 | 0 |
| mt-Cytb | 0 | −1.554026854 | 0.965 | 0.999 | 0 |
| mt-Co1 | 0 | −1.569950123 | 0.956 | 0.999 | 0 |
| Mup21 | 0 | −1.637812283 | 0.302 | 0.929 | 0 |
| Mup11 | 0 | −1.769206667 | 0.949 | 1 | 0 |
| Mup5 | 0 | −1.774609558 | 0.261 | 0.839 | 0 |
| Mup4 | 0 | −1.909428244 | 0.231 | 0.898 | 0 |
| mt-Nd4 | 0 | −1.953205815 | 0.909 | 0.998 | 0 |
| Mup17 | 0 | −2.382123881 | 0.798 | 1 | 0 |
| Mup9 | 0 | −2.520658851 | 0.309 | 0.976 | 0 |
| Mup18 | 0 | −2.585635085 | 0.505 | 0.997 | 0 |
| Mup6 | 0 | −2.588052175 | 0.543 | 0.992 | 0 |
| Mup19 | 0 | −3.348975652 | 0.594 | 1 | 0 |
| Gm23935 | 5.57E−302 | 1.207244314 | 0.997 | 0.925 | 1.38E−297 |
| Vtn | 3.61E−298 | 0.516935534 | 0.998 | 0.983 | 8.96E−294 |
| Cp | 2.34E−294 | 0.655511152 | 0.995 | 0.943 | 5.81E−290 |
| Ugt2b5 | 2.32E−293 | −0.716336886 | 0.923 | 0.959 | 5.77E−289 |
| Cyb5 | 8.57E−293 | −0.751689441 | 0.984 | 0.983 | 2.13E−288 |
| Gm13775 | 2.16E−292 | −1.171528238 | 0.197 | 0.761 | 5.37E−288 |
| Ambp | 2.76E−292 | 0.496213815 | 0.999 | 0.993 | 6.84E−288 |
| Steap4 | 5.28E−289 | 0.931387724 | 0.71 | 0.222 | 1.31E−284 |
| mmu-mir-6236 | 2.07E−288 | 1.257248389 | 0.989 | 0.837 | 5.14E−284 |
| Apoa2 | 4.19E−281 | −0.42773886 | 0.999 | 1 | 1.04E−276 |
| Apcs | 2.39E−279 | 0.965147497 | 0.879 | 0.525 | 5.94E−275 |
| mt-Co2 | 4.87E−279 | −1.39473643 | 0.04 | 0.629 | 1.21E−274 |
| Rbp4 | 1.82E−274 | −0.451760058 | 0.998 | 0.999 | 4.52E−270 |
| Mup15 | 2.40E−272 | −1.359329494 | 0.038 | 0.617 | 5.96E−268 |
| Csad | 2.12E−271 | −1.214863323 | 0.237 | 0.744 | 5.27E−267 |
| Apoa1 | 2.66E−257 | −0.411233725 | 1 | 1 | 6.60E−253 |
| Gsta3 | 1.24E−254 | −0.796926805 | 0.94 | 0.956 | 3.08E−250 |
| Mat1a | 1.17E−250 | 0.519634823 | 0.998 | 0.966 | 2.90E−246 |
| Prg4 | 3.37E−248 | 0.810835665 | 0.643 | 0.188 | 8.36E−244 |
| Serpina3k | 1.13E−246 | −0.298266533 | 1 | 1 | 2.79E−242 |
| Qsox1 | 9.01E−246 | 0.725062115 | 0.93 | 0.707 | 2.24E−241 |
| Lrp1 | 8.74E−236 | 0.914046975 | 0.874 | 0.591 | 2.17E−231 |
| Serpina10 | 5.42E−235 | 0.740578005 | 0.896 | 0.641 | 1.35E−230 |
| Elovl3 | 6.32E−234 | −0.924845607 | 0.48 | 0.827 | 1.57E−229 |
| mt-Co3 | 1.22E−233 | −1.229639242 | 0.097 | 0.603 | 3.03E−229 |
| Tmem176b | 1.30E−230 | 0.713749974 | 0.901 | 0.632 | 3.22E−226 |
| Ahsg | 1.31E−226 | 0.32482682 | 1 | 1 | 3.27E−222 |
| Aldh1l1 | 2.28E−225 | 0.564932962 | 0.989 | 0.904 | 5.65E−221 |
| Sod1 | 1.28E−220 | −0.483374172 | 0.983 | 0.983 | 3.17E−216 |
| Retsat | 1.74E−220 | −0.847260321 | 0.524 | 0.83 | 4.31E−216 |
| Pck1 | 3.48E−214 | 0.495042924 | 0.999 | 0.987 | 8.65E−210 |
| Hrg | 1.60E−213 | 0.58732025 | 0.966 | 0.831 | 3.97E−209 |
| Hc | 9.30E−212 | 0.611232959 | 0.974 | 0.903 | 2.31E−207 |
| Prtn3 | 1.80E−209 | 0.350182853 | 0.271 | 0.003 | 4.48E−205 |
| Scd1 | 1.49E−208 | 0.787835318 | 0.996 | 0.901 | 3.71E−204 |
| Egfr | 1.72E−203 | 0.65440823 | 0.964 | 0.809 | 4.26E−199 |
| Tat | 1.58E−198 | 0.614979826 | 0.988 | 0.903 | 3.93E−194 |
| Orm3 | 4.42E−196 | 0.517250103 | 0.47 | 0.093 | 1.10E−191 |
| Tmem176a | 5.12E−195 | 0.67082422 | 0.773 | 0.429 | 1.27E−190 |
| Igfbp2 | 1.45E−194 | −0.717832911 | 0.644 | 0.885 | 3.60E−190 |
| Ankrd55 | 1.65E−189 | −1.470505577 | 0.312 | 0.696 | 4.09E−185 |
| Cyp2c29 | 1.46E−188 | −0.997502622 | 0.799 | 0.93 | 3.62E−184 |
| Apoc4 | 4.78E−187 | −0.501759591 | 0.98 | 0.98 | 1.19E−182 |
| Sephs2 | 7.48E−187 | 0.604852301 | 0.928 | 0.761 | 1.86E−182 |
| Serping1 | 1.42E−183 | 0.535830748 | 0.964 | 0.87 | 3.54E−179 |
| Il1r1 | 1.89E−181 | 0.63980318 | 0.558 | 0.167 | 4.69E−177 |
| Mpeg1 | 1.71E−178 | 0.541170007 | 0.435 | 0.089 | 4.24E−174 |
| Lbp | 5.79E−176 | 0.656662588 | 0.702 | 0.333 | 1.44E−171 |
| Angptl3 | 1.07E−173 | −0.529458826 | 0.922 | 0.944 | 2.66E−169 |
| Cyp2b10 | 4.98E−173 | −1.247317124 | 0.138 | 0.542 | 1.24E−168 |
| Cox4i1 | 8.99E−173 | −0.518813262 | 0.938 | 0.958 | 2.23E−168 |
| Mup13 | 2.53E−169 | −0.875079323 | 0.129 | 0.546 | 6.29E−165 |
| Rps14 | 1.51E−156 | −0.607507888 | 0.793 | 0.917 | 3.76E−152 |
| Apob | 1.86E−155 | 0.505397578 | 0.997 | 0.989 | 4.63E−151 |
| Cyp2c70 | 2.58E−154 | 0.511802822 | 0.967 | 0.834 | 6.40E−150 |
| Ctsb | 1.82E−153 | 0.553974491 | 0.927 | 0.781 | 4.51E−149 |
| C4bp | 4.59E−153 | 0.505363658 | 0.934 | 0.788 | 1.14E−148 |
| Ifitm3 | 1.15E−152 | 0.506857449 | 0.944 | 0.812 | 2.86E−148 |
| Cox7b | 1.35E−148 | −0.650085753 | 0.747 | 0.878 | 3.36E−144 |
| Tifa | 1.62E−148 | 0.321485305 | 0.297 | 0.036 | 4.02E−144 |
| Cml2 | 2.92E−147 | 0.486040271 | 0.953 | 0.857 | 7.24E−143 |
| Mup7 | 3.24E−146 | −0.844127621 | 0.967 | 0.998 | 8.04E−142 |
| Itih1 | 5.47E−146 | 0.496617676 | 0.936 | 0.816 | 1.36E−141 |
| Myh9 | 1.14E−145 | 0.631035657 | 0.652 | 0.318 | 2.83E−141 |
| Ces2a | 3.16E−145 | −0.619093582 | 0.652 | 0.846 | 7.84E−141 |
| Rps27l | 5.72E−144 | −0.64582348 | 0.684 | 0.849 | 1.42E−139 |
| Inmt | 6.28E−144 | −0.679790467 | 0.791 | 0.884 | 1.56E−139 |
| Cd5l | 1.89E−142 | 0.49371428 | 0.263 | 0.026 | 4.68E−138 |
| mt-Atp6 | 1.26E−138 | −0.875890108 | 0.083 | 0.452 | 3.13E−134 |
| Hspb8 | 1.70E−137 | 0.52383406 | 0.837 | 0.597 | 4.23E−133 |
| P4hb | 5.28E−137 | 0.369961971 | 0.99 | 0.941 | 1.31E−132 |
| Cox6c | 9.25E−137 | −0.610222852 | 0.753 | 0.884 | 2.30E−132 |
| Dhcr24 | 5.84E−135 | 0.551421264 | 0.92 | 0.783 | 1.45E−130 |
| Cyp2c37 | 8.45E−135 | −0.820386118 | 0.239 | 0.608 | 2.10E−130 |
| Mgst1 | 8.85E−134 | −0.36875402 | 0.998 | 0.997 | 2.20E−129 |
| Igfbp4 | 5.12E−133 | 0.411174683 | 0.952 | 0.866 | 1.27E−128 |
| Ang | 1.34E−132 | 0.599634512 | 0.874 | 0.687 | 3.32E−128 |
| Slc41a2 | 1.76E−131 | 0.441321771 | 0.446 | 0.134 | 4.36E−127 |
| Creg1 | 6.09E−130 | −0.480113825 | 0.87 | 0.926 | 1.51E−125 |
| Uqcrq | 9.76E−129 | −0.485354968 | 0.922 | 0.951 | 2.42E−124 |
| Serpina3c | 1.89E−128 | −0.595568461 | 0.042 | 0.385 | 4.68E−124 |
| Comt | 8.75E−128 | 0.384473508 | 0.987 | 0.931 | 2.17E−123 |
| Akr1c6 | 1.28E−127 | −0.457641734 | 0.974 | 0.971 | 3.18E−123 |
| Mup8 | 1.53E−127 | −0.82718199 | 0.033 | 0.373 | 3.80E−123 |
| Cyp8b1 | 6.21E−125 | −0.599290325 | 0.449 | 0.745 | 1.54E−120 |
| Chchd2 | 4.44E−124 | −0.518624159 | 0.844 | 0.916 | 1.10E−119 |
| Clec4f | 6.98E−124 | 0.487336922 | 0.282 | 0.043 | 1.73E−119 |
| Calr | 6.49E−121 | 0.391955529 | 0.961 | 0.885 | 1.61E−116 |
| Ahcy | 6.32E−120 | −0.49011253 | 0.819 | 0.916 | 1.57E−115 |
| Mup2 | 2.15E−119 | −0.72405868 | 0.064 | 0.392 | 5.33E−115 |
| Cyp2c54 | 5.72E−119 | −0.632023168 | 0.475 | 0.759 | 1.42E−114 |
| Ech1 | 9.40E−119 | −0.504022226 | 0.722 | 0.856 | 2.34E−114 |
| Scara5 | 1.06E−118 | 0.285535495 | 0.301 | 0.054 | 2.62E−114 |
| Cfi | 8.71E−118 | 0.390834066 | 0.97 | 0.905 | 2.16E−113 |
| Mug2 | 1.37E−117 | 0.536577024 | 0.881 | 0.738 | 3.40E−113 |
| Ces1f | 3.73E−116 | −0.533638921 | 0.676 | 0.817 | 9.25E−112 |
| H6pd | 4.05E−116 | 0.491965789 | 0.8 | 0.595 | 1.01E−111 |
| Gas6 | 7.61E−115 | 0.269480866 | 0.253 | 0.036 | 1.89E−110 |
| mt-Nd6 | 1.11E−114 | −0.685505467 | 0.245 | 0.577 | 2.74E−110 |
| Hnf4a | 1.24E−114 | 0.470873585 | 0.887 | 0.743 | 3.08E−110 |
| Slc39a14 | 4.67E−113 | 0.506662289 | 0.638 | 0.343 | 1.16E−108 |
| Ndufa4 | 1.67E−111 | −0.512722876 | 0.803 | 0.889 | 4.13E−107 |
| Ugt2b34 | 4.49E−111 | −0.500802828 | 0.776 | 0.874 | 1.12E−106 |
| H2-D1 | 4.57E−110 | 0.433183706 | 0.911 | 0.774 | 1.14E−105 |
| Lpl | 7.13E−110 | 0.355011922 | 0.194 | 0.016 | 1.77E−105 |
| Mup10 | 1.62E−109 | 0.254194035 | 0.893 | 0.692 | 4.03E−105 |
| Ndufb9 | 4.31E−109 | −0.483703094 | 0.78 | 0.888 | 1.07E−104 |
| Rbp1 | 6.28E−109 | 0.32795786 | 0.308 | 0.069 | 1.56E−104 |
| Dap | 2.21E−107 | 0.465257779 | 0.842 | 0.654 | 5.49E−103 |
| Col18a1 | 2.40E−107 | 0.459095159 | 0.903 | 0.79 | 5.95E−103 |
| Tdo2 | 1.13E−106 | −0.283022598 | 0.991 | 0.991 | 2.80E−102 |
| Wfdc17 | 5.33E−106 | 0.317329894 | 0.195 | 0.018 | 1.32E−101 |
| Hint1 | 3.71E−105 | −0.487016283 | 0.737 | 0.858 | 9.22E−101 |
| Cpb2 | 1.34E−104 | 0.403790403 | 0.946 | 0.813 | 3.34E−100 |
| C1qb | 2.56E−104 | 0.337494489 | 0.208 | 0.024 | 6.35E−100 |
| Rpl13a | 9.02E−104 | −0.480174283 | 0.747 | 0.868 | 2.24E−99 |
| Cyp3a25 | 1.85E−103 | −0.416456818 | 0.851 | 0.919 | 4.60E−99 |
| Hspa5 | 4.12E−103 | 0.339769129 | 0.991 | 0.944 | 1.02E−98 |
| Stat3 | 7.07E−103 | 0.483904634 | 0.602 | 0.318 | 1.75E−98 |
| Fetub | 7.47E−103 | 0.425407474 | 0.866 | 0.703 | 1.86E−98 |
| Nnmt | 2.70E−102 | 0.508818069 | 0.877 | 0.716 | 6.69E−98 |
| C8a | 1.21E−100 | 0.404198047 | 0.964 | 0.887 | 3.00E−96 |
| Hsp90ab1 | 4.69E−99 | 0.323037124 | 0.977 | 0.922 | 1.17E−94 |
| Lyz2 | 1.34E−97 | 0.402735723 | 0.222 | 0.032 | 3.33E−93 |
| Slco1a1 | 2.66E−96 | −0.542407867 | 0.584 | 0.777 | 6.62E−92 |
| Ephx2 | 4.78E−96 | −0.398908219 | 0.874 | 0.937 | 1.19E−91 |
| Mup1 | 7.17E−96 | −0.882906956 | 0.306 | 0.583 | 1.78E−91 |
| Hyou1 | 1.36E−95 | 0.496156079 | 0.65 | 0.385 | 3.39E−91 |
| Gstp1 | 1.73E−94 | −0.671664785 | 0.644 | 0.822 | 4.28E−90 |
| Isyna1 | 1.08E−93 | 0.270110032 | 0.218 | 0.034 | 2.67E−89 |
| Slc25a15 | 1.65E−93 | 0.393990477 | 0.896 | 0.738 | 4.09E−89 |
| Scrpinf2 | 3.06E−92 | 0.361403772 | 0.944 | 0.843 | 7.60E−88 |
| Etnppl | 6.39E−92 | −0.566620392 | 0.312 | 0.606 | 1.59E−87 |
| H2-Q10 | 1.82E−91 | 0.250632773 | 0.999 | 0.982 | 4.52E−87 |
| Ugt2b36 | 1.93E−91 | −0.355795521 | 0.924 | 0.93 | 4.78E−87 |
| Hrsp12 | 2.78E−91 | −0.395396316 | 0.898 | 0.939 | 6.91E−87 |
| Acsl1 | 5.10E−90 | −0.417030322 | 0.855 | 0.924 | 1.27E−85 |
| Ndufb6 | 1.60E−89 | −0.518384931 | 0.603 | 0.759 | 3.96E−85 |
| Slc25a25 | 8.79E−89 | 0.442662636 | 0.866 | 0.704 | 2.18E−84 |
| Gnmt | 3.48E−88 | −0.288175489 | 0.996 | 0.988 | 8.65E−84 |
| Uqcrh | 2.37E−87 | −0.483113531 | 0.66 | 0.81 | 5.89E−83 |
| Aldh2 | 2.96E−86 | 0.296492218 | 0.992 | 0.973 | 7.35E−82 |
| Kng2 | 7.60E−86 | 0.368809413 | 0.833 | 0.684 | 1.89E−81 |
| Serpina1a | 2.51E−85 | −0.295262554 | 0.986 | 0.991 | 6.23E−81 |
| Cyp3a13 | 2.58E−85 | 0.420680082 | 0.774 | 0.569 | 6.41E−81 |
| Sc4mol | 4.25E−85 | −0.519568919 | 0.409 | 0.642 | 1.06E−80 |
| Slc38a3 | 1.97E−84 | 0.353500212 | 0.936 | 0.858 | 4.90E−80 |
| Igfbp1 | 4.63E−83 | 0.488899754 | 0.849 | 0.717 | 1.15E−78 |
| Atp5g3 | 7.53E−83 | −0.357248453 | 0.841 | 0.911 | 1.87E−78 |
| Cyp2c50 | 9.57E−83 | −0.516529985 | 0.762 | 0.888 | 2.38E−78 |
| Atp5f1 | 4.44E−82 | −0.428496879 | 0.827 | 0.869 | 1.10E−77 |
| Cps1 | 3.38E−81 | 0.312297912 | 0.991 | 0.971 | 8.39E−77 |
| Cyp2d9 | 6.27E−81 | −0.3250388 | 0.959 | 0.959 | 1.56E−76 |
| Fabp2 | 1.06E−80 | −0.527186942 | 0.377 | 0.622 | 2.63E−76 |
| Uroc1 | 1.61E−80 | 0.459322934 | 0.782 | 0.615 | 3.99E−76 |
| F5 | 4.28E−80 | 0.436709177 | 0.84 | 0.728 | 1.06E−75 |
| Ldhd | 4.17E−79 | 0.387086477 | 0.766 | 0.575 | 1.04E−74 |
| Sdc4 | 8.45E−79 | 0.315436107 | 0.956 | 0.867 | 2.10E−74 |
| 2810007J24Rik | 3.11E−78 | −0.337772907 | 0.919 | 0.943 | 7.72E−74 |
| Hsph1 | 7.72E−78 | 0.425977247 | 0.512 | 0.255 | 1.92E−73 |
| Prdx1 | 9.13E−78 | −0.353764247 | 0.914 | 0.927 | 2.27E−73 |
| Rpl32 | 1.78E−77 | −0.498592915 | 0.574 | 0.757 | 4.42E−73 |
| Hagh | 1.79E−77 | −0.398106396 | 0.799 | 0.858 | 4.45E−73 |
| Selenbp2 | 2.34E−77 | −0.354512086 | 0.905 | 0.937 | 5.81E−73 |
| Fmo5 | 2.40E−77 | 0.337493236 | 0.927 | 0.837 | 5.95E−73 |
| Eef2 | 4.10E−77 | 0.321705764 | 0.937 | 0.873 | 1.02E−72 |
| Ugt2a3 | 1.19E−76 | −0.457903234 | 0.637 | 0.773 | 2.95E−72 |
| Pcx | 1.34E−76 | 0.367136085 | 0.832 | 0.661 | 3.33E−72 |
| Fah | 6.06E−76 | 0.325518495 | 0.926 | 0.834 | 1.51E−71 |
| Hdlbp | 1.02E−75 | 0.394030651 | 0.856 | 0.713 | 2.52E−71 |
| Tmsb4x | 5.25E−75 | 0.341035762 | 0.24 | 0.057 | 1.30E−70 |
| Gjb2 | 7.57E−75 | 0.402811227 | 0.763 | 0.572 | 1.88E−70 |
| Ndufb11 | 8.48E−75 | −0.479044618 | 0.56 | 0.735 | 2.10E−70 |
| Bhlhe40 | 4.96E−74 | 0.370112004 | 0.5 | 0.251 | 1.23E−69 |
| Glud1 | 5.22E−74 | 0.307823999 | 0.972 | 0.905 | 1.30E−69 |
| Cyp2a5 | 7.78E−74 | −0.869524548 | 0.311 | 0.555 | 1.93E−69 |
| Aadac | 2.54E−73 | −0.466442698 | 0.84 | 0.86 | 6.31E−69 |
| Xdh | 5.78E−73 | 0.373181784 | 0.507 | 0.258 | 1.44E−68 |
| Acox1 | 7.26E−73 | 0.292890336 | 0.986 | 0.966 | 1.80E−68 |
| Btg2 | 1.08E−72 | 0.386164963 | 0.484 | 0.241 | 2.69E−68 |
| Mpc2 | 1.09E−72 | −0.39520967 | 0.763 | 0.857 | 2.71E−68 |
| Sar1b | 2.65E−72 | −0.422088437 | 0.655 | 0.772 | 6.58E−68 |
| Atp5h | 3.22E−72 | −0.555498653 | 0.496 | 0.682 | 7.99E−68 |
| Mup14 | 3.43E−72 | −0.741008747 | 0.407 | 0.616 | 8.52E−68 |
| Rps2 | 1.31E−71 | −0.474519664 | 0.579 | 0.757 | 3.26E−67 |
| Abhd2 | 5.22E−71 | 0.267241512 | 0.313 | 0.105 | 1.30E−66 |
| Atp5j2 | 1.30E−70 | −0.409576712 | 0.755 | 0.852 | 3.23E−66 |
| Ugt1a1 | 2.13E−70 | 0.370092558 | 0.526 | 0.283 | 5.30E−66 |
| Atp5j | 2.88E−70 | −0.415380683 | 0.594 | 0.757 | 7.16E−66 |
| Lpgat1 | 4.21E−69 | 0.400787984 | 0.705 | 0.502 | 1.05E−64 |
| Lpin1 | 1.83E−68 | 0.382955017 | 0.552 | 0.31 | 4.53E−64 |
| Cyr61 | 3.89E−68 | 0.302881921 | 0.3 | 0.102 | 9.66E−64 |
| Cfhr2 | 4.10E−68 | −0.428849834 | 0.845 | 0.868 | 1.02E−63 |
| Slc7a2 | 1.08E−66 | 0.374380964 | 0.846 | 0.735 | 2.68E−62 |
| Uqcr11 | 3.45E−65 | −0.368070557 | 0.805 | 0.879 | 8.58E−61 |
| Gstm1 | 6.97E−65 | −0.35224443 | 0.898 | 0.935 | 1.73E−60 |
| Pros1 | 1.21E−64 | 0.26812884 | 0.38 | 0.158 | 3.01E−60 |
| Slc22a7 | 5.79E−64 | 0.307857498 | 0.405 | 0.186 | 1.44E−59 |
| Tgm2 | 1.02E−63 | 0.342526865 | 0.487 | 0.258 | 2.52E−59 |
| Cox7a2 | 1.32E−63 | −0.441101884 | 0.686 | 0.784 | 3.27E−59 |
| Ndufa6 | 1.53E−63 | −0.418444742 | 0.71 | 0.798 | 3.80E−59 |
| Il6st | 1.35E−62 | 0.382881893 | 0.503 | 0.273 | 3.35E−58 |
| Hsd3b5 | 1.66E−62 | −0.506259792 | 0.377 | 0.587 | 4.13E−58 |
| Pon1 | 9.77E−62 | −0.32604762 | 0.857 | 0.906 | 2.43E−57 |
| Ctss | 1.08E−61 | 0.270183132 | 0.137 | 0.019 | 2.68E−57 |
| Plxnb2 | 1.84E−61 | 0.381208682 | 0.54 | 0.315 | 4.58E−57 |
| Gne | 1.85E−61 | 0.355262513 | 0.673 | 0.469 | 4.60E−57 |
| Abcb11 | 2.10E−61 | 0.351857633 | 0.877 | 0.791 | 5.21E−57 |
| Cpn2 | 2.42E−61 | 0.349996687 | 0.795 | 0.667 | 6.01E−57 |
| Rps3 | 2.86E−61 | −0.339903958 | 0.811 | 0.874 | 7.09E−57 |
| Gm4076 | 3.05E−60 | −0.342316728 | 0.007 | 0.184 | 7.57E−56 |
| Ndufb8 | 5.90E−60 | −0.389897205 | 0.594 | 0.746 | 1.46E−55 |
| Slc3a1 | 1.04E−59 | 0.381791335 | 0.393 | 0.189 | 2.59E−55 |
| Lgals9 | 1.46E−59 | 0.327550674 | 0.912 | 0.793 | 3.63E−55 |
| Mtss1 | 4.52E−59 | 0.339863924 | 0.649 | 0.454 | 1.12E−54 |
| Hamp2 | 7.68E−59 | 0.337201585 | 0.231 | 0.07 | 1.91E−54 |
| Thrsp | 1.73E−58 | −0.629247506 | 0.503 | 0.627 | 4.31E−54 |
| Etfb | 1.94E−58 | −0.270014172 | 0.925 | 0.94 | 4.81E−54 |
| Psap | 2.13E−58 | 0.318472244 | 0.709 | 0.513 | 5.30E−54 |
| Mafb | 4.64E−58 | 0.356246663 | 0.553 | 0.33 | 1.15E−53 |
| Cd36 | 9.49E−58 | 0.291074527 | 0.318 | 0.123 | 2.36E−53 |
| Pdia3 | 1.35E−57 | 0.276156858 | 0.879 | 0.797 | 3.36E−53 |
| Habp2 | 1.41E−57 | 0.313462443 | 0.61 | 0.413 | 3.50E−53 |
| Hmgcs1 | 2.77E−57 | −0.387891871 | 0.552 | 0.703 | 6.89E−53 |
| Hsd11b1 | 2.80E−57 | −0.332361427 | 0.85 | 0.875 | 6.95E−53 |
| Abca1 | 8.71E−57 | 0.370856836 | 0.594 | 0.377 | 2.16E−52 |
| Cyp2c55 | 8.91E−57 | −0.347841415 | 0.05 | 0.243 | 2.21E−52 |
| Apon | 1.18E−56 | 0.318989204 | 0.811 | 0.666 | 2.92E−52 |
| Nbr1 | 1.91E−56 | 0.337157409 | 0.613 | 0.413 | 4.73E−52 |
| Slc25a23 | 1.74E−55 | 0.332651576 | 0.603 | 0.399 | 4.31E−51 |
| Malat1 | 1.79E−55 | −0.41172143 | 0.568 | 0.78 | 4.45E−51 |
| Gm26917 | 2.88E−55 | 0.443747552 | 0.374 | 0.181 | 7.15E−51 |
| Leap2 | 5.13E−55 | −0.404792721 | 0.438 | 0.632 | 1.27E−50 |
| Cyp4a10 | 3.93E−54 | −0.453214203 | 0.455 | 0.657 | 9.75E−50 |
| F9 | 7.01E−54 | 0.303980935 | 0.784 | 0.632 | 1.74E−49 |
| Mtus1 | 1.70E−53 | 0.385483144 | 0.615 | 0.42 | 4.23E−49 |
| Kmo | 1.83E−53 | 0.305370487 | 0.836 | 0.732 | 4.54E−49 |
| Iigp1 | 1.99E−53 | 0.305889089 | 0.861 | 0.759 | 4.95E−49 |
| Agt | 2.00E−53 | 0.258205872 | 0.891 | 0.817 | 4.95E−49 |
| Gns | 3.76E−53 | 0.323226146 | 0.497 | 0.288 | 9.33E−49 |
| Serpina3m | 1.13E−52 | 0.305171041 | 0.835 | 0.722 | 2.80E−48 |
| Cth | 1.59E−52 | −0.339209942 | 0.806 | 0.838 | 3.95E−48 |
| Bsg | 2.32E−52 | 0.273171859 | 0.892 | 0.797 | 5.75E−48 |
| Scarb1 | 2.46E−52 | 0.32724782 | 0.552 | 0.358 | 6.10E−48 |
| Got1 | 2.96E−52 | 0.331700716 | 0.753 | 0.587 | 7.35E−48 |
| Grn | 4.53E−52 | 0.316049742 | 0.597 | 0.396 | 1.12E−47 |
| Uqcr10 | 7.78E−52 | −0.382321788 | 0.679 | 0.8 | 1.93E−47 |
| Aass | 1.49E−51 | 0.312355615 | 0.836 | 0.716 | 3.70E−47 |
| Arhgef26 | 1.82E−51 | 0.276634335 | 0.406 | 0.203 | 4.52E−47 |
| Man2a1 | 4.79E−51 | 0.323273082 | 0.741 | 0.583 | 1.19E−46 |
| Akr1c19 | 5.32E−51 | −0.38253326 | 0.342 | 0.527 | 1.32E−46 |
| Ddt | 5.65E−51 | −0.345838673 | 0.703 | 0.829 | 1.40E−46 |
| Hsd17b6 | 3.80E−50 | −0.364685233 | 0.224 | 0.453 | 9.45E−46 |
| Gm9843 | 3.82E−50 | −0.396322103 | 0.112 | 0.299 | 9.49E−46 |
| Eef1b2 | 7.65E−50 | −0.367328377 | 0.693 | 0.78 | 1.90E−45 |
| Serpina12 | 7.73E−50 | −0.404557025 | 0.273 | 0.492 | 1.92E−45 |
| Sardh | 1.19E−49 | 0.28626336 | 0.911 | 0.838 | 2.96E−45 |
| Esd | 2.03E−49 | −0.338909518 | 0.682 | 0.784 | 5.03E−45 |
| Gm10250 | 4.47E−49 | −0.336847064 | 0.074 | 0.251 | 1.11E−44 |
| Slc38a2 | 5.38E−49 | 0.333158401 | 0.609 | 0.413 | 1.34E−44 |
| Edem1 | 7.34E−49 | 0.301212478 | 0.614 | 0.429 | 1.82E−44 |
| Abat | 9.75E−49 | 0.305043176 | 0.808 | 0.677 | 2.42E−44 |
| Creld2 | 2.38E−48 | 0.307177214 | 0.499 | 0.296 | 5.90E−44 |
| Ifitm2 | 3.24E−48 | 0.281474354 | 0.538 | 0.347 | 8.05E−44 |
| Cyp2d10 | 3.72E−48 | −0.26237727 | 0.857 | 0.921 | 9.23E−44 |
| Dpyd | 3.81E−48 | 0.315635393 | 0.792 | 0.659 | 9.45E−44 |
| Ces1e | 3.91E−48 | −0.375733204 | 0.391 | 0.576 | 9.70E−44 |
| Tnfrsf1a | 7.65E−48 | 0.267537895 | 0.447 | 0.244 | 1.90E−43 |
| Alas1 | 8.49E−48 | 0.300367757 | 0.867 | 0.729 | 2.11E−43 |
| Chchd10 | 2.10E−47 | −0.28090926 | 0.935 | 0.944 | 5.22E−43 |
| Cox5b | 2.10E−47 | −0.373464063 | 0.569 | 0.726 | 5.23E−43 |
| Myo18a | 5.21E−47 | 0.253700048 | 0.409 | 0.211 | 1.29E−42 |
| Sparcl1 | 8.10E−47 | −0.253616986 | 0 | 0.136 | 2.01E−42 |
| St6gal1 | 1.18E−46 | 0.295334926 | 0.417 | 0.226 | 2.92E−42 |
| Tapbp | 1.11E−45 | 0.265071778 | 0.464 | 0.263 | 2.77E−41 |
| Cox6a1 | 1.73E−45 | −0.275276508 | 0.833 | 0.887 | 4.29E−41 |
| Rps24 | 2.78E−45 | −0.386464837 | 0.472 | 0.637 | 6.91E−41 |
| Ifih1 | 4.04E−45 | 0.264296168 | 0.383 | 0.195 | 1.00E−40 |
| Copa | 1.91E−44 | 0.294256247 | 0.559 | 0.363 | 4.75E−40 |
| B3galt1 | 3.47E−44 | 0.251993915 | 0.343 | 0.163 | 8.61E−40 |
| BC025446 | 1.79E−43 | 0.266911384 | 0.497 | 0.306 | 4.46E−39 |
| Pcdh1 | 3.18E−43 | 0.265458741 | 0.411 | 0.222 | 7.89E−39 |
| Eif4g2 | 3.25E−43 | 0.287105046 | 0.807 | 0.678 | 8.07E−39 |
| Ephx1 | 1.05E−42 | −0.332038055 | 0.694 | 0.8 | 2.60E−38 |
| Acot1 | 1.07E−42 | −0.299327096 | 0.07 | 0.237 | 2.65E−38 |
| 1300002K09Rik | 7.15E−42 | 0.30148927 | 0.673 | 0.514 | 1.78E−37 |
| Plec | 8.20E−42 | 0.267122123 | 0.364 | 0.186 | 2.04E−37 |
| Rpl14 | 8.70E−42 | −0.357060958 | 0.403 | 0.574 | 2.16E−37 |
| Caprin1 | 1.14E−41 | 0.295892842 | 0.644 | 0.481 | 2.84E−37 |
| Gm5096 | 3.78E−41 | −0.350534749 | 0.291 | 0.468 | 9.38E−37 |
| Uba1 | 7.69E−41 | 0.281371963 | 0.579 | 0.416 | 1.91E−36 |
| Hacl1 | 1.10E−40 | −0.294916761 | 0.749 | 0.797 | 2.73E−36 |
| Kynu | 1.18E−40 | 0.28095477 | 0.728 | 0.588 | 2.94E−36 |
| Rxra | 2.31E−40 | 0.264560061 | 0.713 | 0.574 | 5.74E−36 |
| Tfr2 | 3.41E−40 | 0.268628506 | 0.761 | 0.633 | 8.48E−36 |
| Ctnnd1 | 5.70E−40 | 0.252846161 | 0.398 | 0.217 | 1.41E−35 |
| Zfp683 | 1.26E−39 | −0.421689112 | 0 | 0.116 | 3.13E−35 |
| Fndc3a | 2.93E−39 | 0.273177056 | 0.458 | 0.277 | 7.28E−35 |
| Cpt1a | 4.79E−39 | 0.276570679 | 0.763 | 0.651 | 1.19E−34 |
| Rps18 | 4.80E−39 | −0.415076007 | 0.422 | 0.578 | 1.19E−34 |
| Gm20594 | 5.36E−39 | −0.496964367 | 0.131 | 0.294 | 1.33E−34 |
| 1110008F13Rik | 5.70E−39 | −0.327482854 | 0.51 | 0.648 | 1.42E−34 |
| Glo1 | 6.38E−39 | −0.317338548 | 0.644 | 0.749 | 1.59E−34 |
| Adh4 | 7.50E−39 | 0.282357284 | 0.676 | 0.521 | 1.86E−34 |
| Cyp3a44 | 9.72E−39 | −0.297417766 | 0.043 | 0.183 | 2.41E−34 |
| Ugt2b1 | 6.39E−38 | −0.261758027 | 0.835 | 0.871 | 1.59E−33 |
| Ndufb4 | 8.62E−38 | −0.331687642 | 0.322 | 0.49 | 2.14E−33 |
| C130074G19Rik | 1.30E−37 | 0.255729195 | 0.603 | 0.439 | 3.22E−33 |
| Ptprf | 1.81E−37 | 0.27768605 | 0.628 | 0.464 | 4.49E−33 |
| Uqcrb | 2.44E−37 | −0.359656695 | 0.508 | 0.641 | 6.07E−33 |
| Arhgef12 | 5.06E−37 | 0.304865834 | 0.573 | 0.408 | 1.26E−32 |
| Sdhd | 9.94E−37 | −0.302302961 | 0.565 | 0.677 | 2.47E−32 |
| Hint2 | 1.37E−36 | −0.33360366 | 0.569 | 0.676 | 3.41E−32 |
| Osbpl9 | 4.27E−36 | 0.270713808 | 0.598 | 0.448 | 1.06E−31 |
| Vwa8 | 6.13E−36 | 0.262040545 | 0.489 | 0.311 | 1.52E−31 |
| Oaz1 | 6.73E−36 | −0.327014451 | 0.679 | 0.75 | 1.67E−31 |
| Cyp51 | 2.31E−35 | −0.324000937 | 0.523 | 0.649 | 5.74E−31 |
| Plcxd2 | 2.57E−35 | 0.266285463 | 0.509 | 0.332 | 6.38E−31 |
| Ndufc2 | 3.99E−35 | −0.371342649 | 0.458 | 0.579 | 9.91E−31 |
| Rps5 | 4.72E−35 | −0.25674004 | 0.84 | 0.873 | 1.17E−30 |
| Ndufb2 | 4.85E−35 | −0.336855533 | 0.491 | 0.629 | 1.21E−30 |
| Serpina6 | 4.89E−35 | 0.269623074 | 0.463 | 0.296 | 1.21E−30 |
| Blvrb | 8.86E−35 | −0.351075319 | 0.409 | 0.564 | 2.20E−30 |
| Eci1 | 1.50E−34 | −0.31335345 | 0.592 | 0.709 | 3.71E−30 |
| Slc10a1 | 1.69E−34 | −0.273026083 | 0.836 | 0.843 | 4.19E−30 |
| Mbl2 | 2.61E−34 | 0.268002193 | 0.924 | 0.829 | 6.49E−30 |
| Rnf125 | 3.23E−34 | −0.310633092 | 0.337 | 0.493 | 8.03E−30 |
| Ndufs6 | 8.29E−34 | −0.341035035 | 0.627 | 0.724 | 2.06E−29 |
| Cnbp | 9.89E−34 | −0.286746587 | 0.71 | 0.778 | 2.46E−29 |
| Atp2a2 | 1.78E−33 | 0.263222967 | 0.645 | 0.495 | 4.42E−29 |
| Cyp4a14 | 4.43E−33 | −0.31778609 | 0.253 | 0.431 | 1.10E−28 |
| Prdx4 | 6.99E−33 | −0.339424675 | 0.491 | 0.607 | 1.74E−28 |
| Pid1 | 9.26E−33 | 0.263310286 | 0.549 | 0.381 | 2.30E−28 |
| Txn1 | 2.60E−32 | −0.277353299 | 0.75 | 0.809 | 6.45E−28 |
| Cmah | 2.64E−32 | 0.251319774 | 0.619 | 0.465 | 6.55E−28 |
| Atf3 | 1.25E−31 | 0.262281273 | 0.327 | 0.179 | 3.11E−27 |
| Baat | 5.96E−31 | −0.265815179 | 0.705 | 0.767 | 1.48E−26 |
| Rpl36al | 1.38E−30 | −0.331844566 | 0.407 | 0.551 | 3.44E−26 |
| Hebp1 | 1.59E−30 | −0.274169133 | 0.663 | 0.75 | 3.96E−26 |
| Aldh1b1 | 5.04E−30 | 0.360480753 | 0.255 | 0.132 | 1.25E−25 |
| Insig2 | 1.14E−29 | −0.275363349 | 0.75 | 0.81 | 2.82E−25 |
| Efna1 | 1.15E−29 | 0.268455179 | 0.451 | 0.309 | 2.85E−25 |
| Gadd45b | 1.76E−29 | −0.301158726 | 0.147 | 0.291 | 4.38E−25 |
| G0s2 | 2.10E−29 | −0.269113384 | 0.183 | 0.33 | 5.23E−25 |
| Fdps | 3.09E−28 | −0.254137438 | 0.293 | 0.445 | 7.67E−24 |
| Aqp8 | 1.90E−27 | −0.273149234 | 0.366 | 0.505 | 4.72E−23 |
| Ssr4 | 3.10E−27 | −0.283161199 | 0.597 | 0.687 | 7.71E−23 |
| Minos1 | 3.22E−27 | −0.308403636 | 0.493 | 0.595 | 8.00E−23 |
| Ndufa2 | 3.89E−27 | −0.308306345 | 0.651 | 0.738 | 9.67E−23 |
| Tmem243 | 8.31E−27 | −0.275109242 | 0.436 | 0.557 | 2.06E−22 |
| Rps21 | 1.13E−26 | −0.316866281 | 0.451 | 0.579 | 2.81E−22 |
| Ndufa5 | 1.69E−26 | −0.310280044 | 0.451 | 0.567 | 4.19E−22 |
| Tubb2a | 2.11E−26 | −0.260491764 | 0.297 | 0.431 | 5.24E−22 |
| Akr1c14 | 4.84E−26 | −0.280169437 | 0.63 | 0.707 | 1.20E−21 |
| Atp5o | 9.05E−26 | −0.268645357 | 0.58 | 0.712 | 2.25E−21 |
| Ppp2r5a | 1.31E−25 | −0.281895906 | 0.485 | 0.594 | 3.25E−21 |
| Clpx | 1.50E−25 | −0.250587235 | 0.732 | 0.783 | 3.74E−21 |
| Psmb1 | 2.29E−25 | −0.271574562 | 0.716 | 0.759 | 5.68E−21 |
| Rps9 | 2.57E−25 | −0.30861002 | 0.554 | 0.675 | 6.38E−21 |
| Nme1 | 3.82E−25 | −0.291609359 | 0.531 | 0.622 | 9.48E−21 |
| Ndufb5 | 5.36E−25 | −0.274484363 | 0.516 | 0.612 | 1.33E−20 |
| Rgn | 1.09E−24 | 0.261094268 | 0.911 | 0.874 | 2.71E−20 |
| Psma6 | 1.13E−24 | −0.281709731 | 0.596 | 0.675 | 2.82E−20 |
| Egr1 | 1.35E−24 | 0.271667356 | 0.565 | 0.447 | 3.36E−20 |
| Rplp1 | 2.78E−24 | −0.274821325 | 0.667 | 0.757 | 6.89E−20 |
| Fam162a | 4.60E−24 | −0.280519821 | 0.41 | 0.53 | 1.14E−19 |
| Rabac1 | 5.66E−24 | −0.275369587 | 0.403 | 0.524 | 1.41E−19 |
| 4931406C07Rik | 1.25E−23 | −0.28178071 | 0.671 | 0.748 | 3.10E−19 |
| Sc5d | 1.59E−23 | −0.274286355 | 0.779 | 0.776 | 3.96E−19 |
| Bnip3 | 5.74E−23 | −0.272700001 | 0.43 | 0.55 | 1.43E−18 |
| Timm13 | 5.78E−23 | −0.266267617 | 0.567 | 0.671 | 1.44E−18 |
| Ndufs4 | 9.09E−23 | −0.299960018 | 0.476 | 0.562 | 2.26E−18 |
| Selk | 1.84E−22 | −0.279248941 | 0.378 | 0.486 | 4.58E−18 |
| Ifi27 | 2.17E−22 | 0.274586258 | 0.565 | 0.453 | 5.38E−18 |
| Cyp1a2 | 3.26E−22 | −0.284567418 | 0.726 | 0.794 | 8.09E−18 |
| Mrps18c | 4.36E−22 | −0.27427115 | 0.273 | 0.394 | 1.08E−17 |
| Rpl13 | 1.42E−21 | −0.282418264 | 0.349 | 0.464 | 3.52E−17 |
| Rps8 | 1.02E−20 | −0.279845708 | 0.36 | 0.471 | 2.52E−16 |
| Sco2 | 1.24E−20 | −0.250104753 | 0.389 | 0.508 | 3.07E−16 |
| Rps15 | 1.38E−20 | −0.324542895 | 0.435 | 0.528 | 3.43E−16 |
| Nlrp12 | 4.33E−20 | −0.269307598 | 0.314 | 0.43 | 1.08E−15 |
| Timm8b | 9.75E−20 | −0.25098905 | 0.42 | 0.525 | 2.42E−15 |
| Slc22a30 | 9.94E−20 | −0.25736876 | 0.397 | 0.493 | 2.47E−15 |
| Dhrs4 | 2.40E−19 | −0.268862728 | 0.604 | 0.69 | 5.96E−15 |
| Rps10 | 1.55E−17 | −0.252659935 | 0.348 | 0.453 | 3.85E−13 |
| Rpl34 | 5.76E−17 | −0.264586248 | 0.299 | 0.398 | 1.43E−12 |
| Hspe1 | 1.26E−16 | −0.257910168 | 0.467 | 0.557 | 3.13E−12 |
| Ftl1 | 1.88E−15 | −0.347806606 | 0.328 | 0.409 | 4.66E−11 |
| Usmg5 | 4.44E−15 | −0.28365494 | 0.346 | 0.43 | 1.10E−10 |
| Cebpb | 1.24E−14 | −0.267669957 | 0.38 | 0.45 | 3.07E−10 |
| Gulo | 1.57E−14 | 0.282891961 | 0.573 | 0.497 | 3.90E−10 |
| Rpl26 | 5.46E−14 | −0.264487941 | 0.463 | 0.53 | 1.36E−09 |
| Rps19 | 2.07E−10 | −0.25849266 | 0.409 | 0.474 | 5.15E−06 |
| Cyp2e1 | 2.79E−10 | 0.296899706 | 0.88 | 0.911 | 6.94E−06 |
| Glul | 1.05E−08 | 0.495360357 | 0.25 | 0.18 | 0.000261488 |
| Slc1a2 | 2.46E−07 | 0.262926395 | 0.078 | 0.042 | 0.006115724 |

To identify what cellular programs activate in response to each injury model, clustering and differential expression was performed over APAP and UT clusters and PH and UT clusters. Upregulation of genes unique to each injury model were identified, such as Gclc, a rate limiting enzyme in the synthesis of the anti-oxidant glutathione for APAP.

Tables 8A and 8B provide Composite DEG for partial hepatectomy (PH) and acetaminophen (APAP) treatment. DEG for each individual treatment condition pooled across all time points within PH conditions and all time points within APAP conditions to generate a composite list of all genes which are differentially expressed at any time point relative to untreated for each injury type.

Table 8C shows differentially expressed genes between APAP and PH treatment, with Table 9 providing pathway enrichments unique to APAP and Table 10 the pathway enrichments unique to PH.

TABLE 8A
Differentially expressed genes between treatment (APAP and UT).

| ID | A6logFC | A24logFC | A48logFC | A6p | A24p |
|---|---|---|---|---|---|
| Mup17 | −2.354358557 | −1.800348604 | −2.09635042 | 1.37E−209 | 0 |
| Mup6 | −1.871852037 | −1.678184828 | −2.032084932 | 2.16E−149 | 1.18E−255 |
| Mup9 | −2.004163996 | −1.847446915 | −1.93588422 | 9.10E−177 | 0 |
| Mup19 | −2.396345291 | −1.699352699 | −1.730868177 | 4.33E−227 | 0 |
| Mup11 | −3.213953203 | −1.611434806 | −1.621134615 | 1.04E−235 | 0 |
| Mup18 | −1.627956781 | −1.727178347 | −1.590870011 | 4.19E−157 | 0 |
| Selenbp2 | −1.009165718 | −0.383324562 | −1.190221952 | 6.93E−118 | 5.26E−63 |
| Mup7 | −0.941370494 | NA | −1.115587848 | 1.78E−67 | NA |
| Mup16 | −1.602085539 | −1.018975936 | −1.100681479 | 1.22E−168 | 2.06E−200 |
| mt-Co1 | NA | −1.43342755 | −1.043307563 | NA | 0 |
| Mup21 | 0.827249928 | −0.697735774 | −1.008171912 | 2.40E−75 | 2.15E−121 |
| mt-Nd4 | 0.638848134 | −1.957424825 | −0.936405909 | 7.92E−71 | 0 |
| mt-Nd2 | NA | −1.241926048 | −0.898054789 | NA | 0 |
| mt-Cytb | NA | −1.571232911 | −0.8545555 | NA | 0 |
| mt-Nd5 | NA | −1.383591054 | −0.799147191 | NA | 0 |
| Serpina1e | −0.268413792 | −0.284344569 | −0.662045945 | 1.97E−26 | 8.67E−65 |
| mt-Nd1 | −0.388336084 | −1.419683574 | −0.591646758 | 2.80E−49 | 0 |
| Mup3 | −0.703483173 | −0.806219448 | −0.379596014 | 1.31E−157 | 0 |
| Apoe | −0.368966531 | −0.421271729 | 0.407256897 | 3.19E−71 | 3.30E−238 |
| Ces1c | NA | −0.278292823 | 0.466837177 | NA | 1.32E−78 |
| Hmgcs2 | NA | −0.317281221 | 0.629289122 | NA | 1.04E−73 |
| Angptl3 | NA | NA | 0.7089371 | NA | NA |
| Ang | −0.662883759 | NA | 0.7089371 | 5.46E−47 | NA |
| Chchd10 | −0.40191686 | NA | 0.720691838 | 2.84E−29 | NA |
| Chchd1 | −0.439819068 | NA | 0.720691838 | 4.34E−20 | NA |
| Mat1a | 0.790765268 | NA | 1.000433202 | 3.26E−113 | NA |
| Cyp4a10 | 0.456645767 | −0.742207081 | 1.135370857 | 1.93E−12 | 2.66E−98 |
| Cyp4a14 | 1.122498795 | −0.372863761 | 1.704093543 | 2.33E−95 | 6.77E−27 |
| Hbb-bs | 2.862044849 | 1.042581651 | NA | 0 | 2.58E−59 |
| Hmox1 | 1.347585217 | NA | NA | 0 | NA |
| Gm26924 | 1.16085221 | 1.419027502 | NA | 3.70E−144 | 0 |
| Fga | 0.69335023 | 0.828543313 | NA | 2.44E−115 | 0 |
| n-R5-8s1 | 0.766100662 | 1.252818829 | NA | 1.81E−109 | 0 |
| Fgg | 0.615329068 | 0.680530925 | NA | 1.29E−106 | 0 |
| Fgb | 0.646934246 | 0.744973762 | NA | 4.40E−105 | 0 |
| Grip2 | NA | 0.877868293 | NA | NA | 0 |
| Apoa2 | −0.542784959 | −0.545518454 | NA | 6.54E−122 | 0 |
| Apoh | −0.262314298 | NA | 0.411790107 | 4.51E−22 | NA |
| Ttpa | NA | NA | 0.601240096 | NA | NA |
| Lars2 | 1.391744455 | 1.711400221 | NA | 9.69E−109 | 1.38E−293 |
| Gm13775 | −1.065618856 | −1.032451941 | −1.07808822 | 3.12E−88 | 3.97E−187 |
| Cdo1 | NA | NA | 0.517045053 | NA | NA |
| Gm15564 | 1.443066465 | 1.820580657 | NA | 4.17E−104 | 6.31E−292 |
| S100a9 | 1.342849617 | NA | NA | 5.31E−281 | NA |
| Tnfrsf12a | 1.117506296 | NA | NA | 5.70E−279 | NA |
| Gm24601 | 0.46836359 | 0.984391691 | NA | 4.45E−106 | 2.17E−275 |
| Apoc2 | NA | NA | 0.782126085 | NA | NA |
| Retsat | 0.491096059 | −0.365840188 | 0.681307283 | 4.57E−36 | 3.00E−43 |
| Ttr | −0.530625643 | NA | 0.388907438 | 3.44E−101 | NA |
| Sephs2 | NA | 0.889992859 | NA | NA | 2.83E−263 |
| S100a8 | 1.127879209 | NA | NA | 1.92E−258 | NA |
| Nudt7 | −1.564266936 | NA | −1.14157535 | 1.44E−129 | NA |
| mt-Rnr2 | NA | −0.778341998 | −0.559446336 | NA | 5.04E−251 |
| Ppl | 0.879840886 | NA | NA | 9.97E−249 | NA |
| Serpina1a | −0.378457074 | −0.675201963 | NA | 8.08E−39 | 4.32E−247 |
| Acaa1b | −0.732823459 | −0.85084133 | NA | 9.23E−93 | 4.51E−247 |
| Sepp1 | NA | 0.448586822 | 0.33970689 | NA | 1.35E−231 |
| Srxn1 | 1.878352012 | NA | NA | 2.22E−243 | NA |
| Apoc1 | −0.871282735 | −0.380336769 | 0.392095407 | 1.40E−171 | 2.01E−117 |
| Mup5 | −1.23728834 | −1.370621307 | −1.351810718 | 1.04E−61 | 6.49E−171 |
| Fgl1 | NA | 0.864933885 | NA | NA | 1.60E−238 |
| Gpx1 | −0.768556331 | −0.50797139 | NA | 9.80E−161 | 6.77E−238 |
| Mt2 | 2.06273693 | NA | NA | 1.90E−237 | NA |
| Ankrd55 | −1.250834631 | −1.8735659 | −0.974280427 | 1.82E−54 | 4.38E−235 |
| Mup15 | −1.316464146 | −1.234928375 | −1.26121256 | 1.10E−84 | 1.08E−153 |
| Mfsd2a | 0.666142747 | NA | 0.612232714 | 7.88E−65 | NA |
| Mt1 | 2.387707773 | −0.348193009 | 0.356326784 | 3.51E−232 | 3.61E−18 |
| Ndrg2 | NA | NA | 0.41219937 | NA | NA |
| Ahsg | NA | NA | 0.292489576 | NA | NA |
| Hrg | NA | NA | 0.518895355 | NA | NA |
| mt-Co2 | −1.105332856 | −1.396543228 | −0.918784455 | 3.38E−58 | 7.79E−219 |
| Sult1a1 | 0.318989978 | NA | 0.560453494 | 6.64E−07 | NA |
| Agt | NA | NA | 0.520454244 | NA | NA |
| Cyb5 | −0.71018958 | −0.704808492 | NA | 7.09E−94 | 8.36E−212 |
| Fth1 | NA | NA | 0.378367166 | NA | NA |
| Snord118 | NA | 0.391412496 | 0.617425052 | NA | 4.02E−38 |
| Fabp1 | −1.016174848 | NA | NA | 1.10E−198 | NA |
| Slco1a4 | 0.313739505 | NA | 0.552641329 | 4.36E−10 | NA |
| mt-Rnr1 | NA | −0.456211361 | −0.485399855 | NA | 2.01E−123 |
| Saa1 | NA | 0.558211954 | −1.078220383 | NA | 9.12E−37 |
| Hilpda | 0.778625914 | NA | NA | 3.64E−193 | NA |
| Gclc | 1.902837282 | NA | NA | 1.67E−192 | NA |
| Cp | 0.254076812 | 0.317404056 | 0.436243259 | 2.41E−10 | 1.74E−45 |
| Igfbp4 | NA | NA | 0.43528879 | NA | NA |
| Akr1c6 | −1.586259995 | −0.317638782 | −0.468988487 | 2.54E−187 | 2.86E−52 |
| Rbp4 | NA | NA | 0.301440617 | NA | NA |
| Mup12 | −1.269973728 | NA | −0.767698995 | 4.63E−127 | NA |
| Serpina1d | −0.289011223 | −0.494007182 | NA | 2.07E−26 | 1.74E−184 |
| Hspa5 | 0.268221373 | 0.610549064 | 0.291184144 | 9.35E−08 | 9.07E−184 |
| Mgst1 | −1.060154854 | NA | NA | 2.13E−181 | NA |
| Rdh7 | −0.418625957 | −0.269043262 | 0.364161562 | 7.41E−48 | 1.69E−54 |
| Apoc3 | −0.768781074 | −0.440433124 | 0.327008256 | 1.59E−133 | 3.26E−151 |
| Herpud1 | −0.497586072 | NA | 0.513571228 | 1.98E−36 | NA |
| Aldob | 0.315339576 | NA | 0.365460648 | 1.58E−35 | NA |
| Abcd2 | NA | NA | 0.285090597 | NA | NA |
| Gc | NA | NA | 0.259816642 | NA | NA |
| Abcb4 | NA | NA | 0.494736917 | NA | NA |
| mt-Co3 | −0.705930608 | −1.167027737 | −0.915108473 | 1.72E−25 | 9.57E−168 |
| Ugt2b5 | −0.603351248 | −0.576498765 | NA | 3.90E−81 | 9.60E−175 |
| Slc27a2 | NA | NA | 0.385256962 | NA | NA |
| Ccl6 | 0.78914235 | NA | NA | 1.24E−171 | NA |
| St3gal5 | 0.607639775 | NA | 0.542378437 | 6.13E−38 | NA |
| Hbb-bt | 1.430134393 | 0.364374989 | NA | 2.56E−170 | 3.52E−29 |
| Reep6 | NA | NA | 0.514272451 | NA | NA |
| Slc16a6 | 0.911357108 | NA | NA | 4.06E−168 | NA |
| Ttc36 | −0.660645659 | NA | 0.38128834 | 8.42E−81 | NA |
| Adh1 | −1.255028206 | −0.342018972 | NA | 1.90E−167 | 8.71E−80 |
| Gsta3 | −0.806290466 | −0.66935487 | NA | 6.16E−95 | 2.44E−166 |
| Cvp2e1 | −2.318570161 | −0.499033632 | NA | 4.79E−166 | 3.18E−51 |
| Plin2 | 1.461295758 | NA | NA | 8.58E−163 | NA |
| Il1r1 | 1.244999819 | NA | NA | 1.47E−162 | NA |
| F2 | NA | NA | 0.32133198 | NA | NA |
| Decr1 | NA | NA | 0.466364339 | NA | NA |
| Actb | 1.059835055 | 0.38446276 | −0.294140078 | 6.06E−159 | 5.37E−69 |
| Slc25a47 | 0.428932953 | NA | 0.502661475 | 2.18E−12 | NA |
| Srgn | 0.934067453 | NA | NA | 5.07E−158 | NA |
| Pabpc1 | 0.410051805 | NA | 0.494914201 | 4.92E−19 | NA |
| Cyp2d9 | −0.640698605 | −0.567166215 | NA | 2.15E−73 | 3.96E−157 |
| Hyou1 | NA | 0.846484822 | NA | NA | 1.62E−156 |
| Lcat | NA | NA | 0.436082968 | NA | NA |
| Grn | 0.540768685 | NA | 0.473252349 | 1.38E−25 | NA |
| Hsd3b5 | −1.069520224 | NA | −0.830145941 | 2.04E−80 | NA |
| Ube2r2 | NA | NA | 0.46659877 | NA | NA |
| Hamp | −1.02112368 | −0.826148443 | NA | 2.14E−94 | 2.59E−153 |
| Apoa5 | NA | −0.298987336 | 0.323188383 | NA | 3.48E−59 |
| Cdkn1a | 1.2152557 | NA | NA | 2.99E−152 | NA |
| Serpina1c | NA | −0.402626732 | NA | NA | 7.17E−151 |
| Gstp1 | −0.264374672 | −0.430197368 | −0.855831436 | 0.00132565 | 1.39E−27 |
| Tubb6 | 0.576687679 | NA | NA | 6.13E−150 | NA |
| Apoc4 | −1.002265767 | −0.428496936 | NA | 9.54E−150 | 3.56E−114 |
| Nid1 | 0.342599117 | NA | NA | 1.04E−149 | NA |
| Calr | NA | 0.555145472 | NA | NA | 1.67E−148 |
| Klf6 | 0.732407935 | NA | NA | 1.50E−147 | NA |
| Cers6 | 0.532818648 | NA | NA | 8.16E−146 | NA |
| Hmgcl | NA | NA | 0.473794522 | NA | NA |
| Aldh9a1 | NA | NA | 0.415934269 | NA | NA |
| Txnrd1 | 1.443578942 | 0.260866185 | NA | 1.43E−144 | 1.44E−15 |
| Il1b | 0.742893545 | NA | NA | 1.02E−143 | NA |
| Hspa8 | NA | −0.359978165 | 0.420379148 | NA | 1.12E−41 |
| Psat1 | 0.301248562 | NA | NA | 8.43E−143 | NA |
| Gm23935 | 0.424222263 | 0.721300311 | 0.50194422 | 1 | 0.068577969 |
| Tmbim6 | −0.271070995 | NA | 0.338652766 | 2.03E−15 | NA |
| Rgn | −1.573093006 | NA | NA | 9.97E−141 | NA |
| Amy1 | −0.282967905 | NA | 0.432738347 | 2.05E−09 | NA |
| Uqcrq | −0.88665513 | −0.595921048 | NA | 4.23E−106 | 1.50E−140 |
| Scd1 | −0.466125013 | 0.726687286 | 0.519218079 | 5.04E−19 | 2.08E−139 |
| Itih4 | NA | 0.525074867 | NA | NA | 4.26E−137 |
| Gyk | 0.368418571 | NA | 0.402444184 | 5.71E−07 | NA |
| Hp | NA | 0.431328341 | NA | NA | 8.95E−137 |
| Chka | 1.375010255 | NA | NA | 2.35E−136 | NA |
| Pdia3 | NA | 0.593769188 | NA | NA | 7.36E−136 |
| Decr2 | NA | NA | 0.418851877 | NA | NA |
| Cyp3a25 | −0.386094858 | NA | 0.318573266 | 1.37E−27 | NA |
| Serping1 | NA | NA | 0.41280952 | NA | NA |
| Wbp1l | 0.553507326 | NA | 0.440462928 | 2.48E−26 | NA |
| Hsp90b1 | −0.261479921 | 0.534208217 | NA | 2.81E−10 | 6.37E−134 |
| Gnmt | −0.33340449 | −0.46036106 | NA | 1.40E−31 | 1.33E−133 |
| Atp5k | −0.47725773 | NA | 0.408878378 | 2.10E−29 | NA |
| 2010003K11Rik | 0.588354735 | NA | NA | 2.61E−131 | NA |
| Serpina3m | 0.967441963 | NA | 0.432583404 | 2.50E−94 | NA |
| Slco1b2 | −1.087378176 | NA | 0.386023002 | 3.68E−129 | NA |
| Lipa | NA | NA | 0.41833763 | NA | NA |
| Mup20 | NA | −0.281871664 | −0.476207303 | NA | 9.36E−26 |
| Rgs16 | NA | NA | 0.322559157 | NA | NA |
| Bhmt | −0.311413339 | −0.520184894 | NA | 5.24E−16 | 1.20E−127 |
| Vtn | NA | NA | 0.333811366 | NA | NA |
| Plin5 | 0.296281512 | −0.277759837 | 0.352087102 | 6.88E−09 | 2.38E−16 |
| C3 | 0.44276078 | 0.449521065 | 0.259166718 | 1.55E−58 | 1.23E−125 |
| Agpat6 | NA | NA | 0.396260224 | NA | NA |
| Acat1 | NA | NA | 0.368871999 | NA | NA |
| Ets2 | 0.695623802 | NA | NA | 2.30E−125 | NA |
| Mup4 | −0.99530818 | −1.087038527 | −0.953101828 | 1.26E−43 | 1.96E−121 |
| Atp5h | −0.6398091 | −0.579498914 | 0.434163543 | 8.64E−40 | 7.16E−69 |
| Car3 | −1.036093133 | NA | −0.405308591 | 1.61E−124 | NA |
| Acbd5 | −0.3952179 | NA | 0.385608793 | 2.30E−17 | NA |
| Nr1i3 | −0.412654588 | −0.282083592 | 0.425883248 | 1.00E−16 | 1.07E−16 |
| Crat | NA | NA | 0.255626614 | NA | NA |
| Slc27a5 | NA | NA | 0.336994904 | NA | NA |
| Oaf | −0.7328674 | NA | 0.403567608 | 4.77E−65 | NA |
| Cyp3a11 | −0.423130745 | −0.363891177 | 0.313927334 | 1.90E−61 | 1.05E−122 |
| Krt18 | 1.257881489 | NA | NA | 3.97E−121 | NA |
| Elovl3 | −0.506724135 | −0.675967012 | −0.442652848 | 4.74E−35 | 6.30E−121 |
| Cox5a | NA | NA | 0.40236048 | NA | NA |
| Ahcy | NA | −0.562968597 | NA | NA | 6.43E−120 |
| Sdhc | NA | NA | 0.394392518 | NA | NA |
| Gstt3 | NA | NA | 0.350739865 | NA | NA |
| Asl | 0.558638879 | NA | 0.438458339 | 8.13E−37 | NA |
| Atp5b | NA | NA | 0.291727476 | NA | NA |
| C9 | −0.395507776 | NA | −0.427055258 | 5.97E−29 | NA |
| Cyp2b10 | −0.517460658 | −1.164490931 | NA | 2.09E−08 | 9.40E−117 |
| 1100001G20Rik | −0.851607217 | NA | NA | 1.14E−116 | NA |
| Slc25a20 | NA | NA | 0.380017899 | NA | NA |
| Hba-a1 | 0.734957385 | NA | NA | 2.58E−115 | NA |
| Ildr2 | NA | NA | 0.267607713 | NA | NA |
| Ppara | −0.287133273 | NA | 0.35885744 | 1.36E−08 | NA |
| Dusp5 | 0.296374788 | NA | NA | 1.01E−114 | NA |
| Mup8 | −0.752577709 | −0.613334367 | −0.79575704 | 2.07E−27 | 4.14E−25 |
| P4hb | NA | 0.405223744 | 0.250660872 | NA | 2.27E−113 |
| 2810007J24Rik | −0.914301122 | NA | NA | 6.76E−113 | NA |
| Rpl38 | NA | NA | 0.254329206 | NA | NA |
| Tgoln1 | 0.265747623 | NA | 0.38833408 | 9.27E−05 | NA |
| Cox4i1 | −0.515989169 | −0.454243631 | NA | 5.83E−60 | 4.62E−112 |
| Brap | NA | NA | 0.349229859 | NA | NA |
| Cox8a | −0.399332537 | NA | 0.44171223 | 1.73E−19 | NA |
| Bfar | NA | NA | 0.290332897 | NA | NA |
| D17Wsu92e | 0.252482331 | NA | 0.371951808 | 0.259418286 | NA |
| Glud1 | NA | 0.451048591 | NA | NA | 6.55E−110 |
| Slc40a1 | 0.466969696 | NA | 0.356026755 | 6.46E−08 | NA |
| Ephx2 | NA | −0.369303553 | 0.281258562 | NA | 3.71E−68 |
| Bnip3 | −0.336168189 | −0.292803703 | 0.403206563 | 1.17E−13 | 2.86E−22 |
| Rnase4 | −1.049196753 | 0.517836403 | NA | 1.89E−107 | 3.56E−82 |
| Dhcr24 | NA | 0.423756767 | 0.387274317 | NA | 3.44E−48 |
| Myh9 | 1.160233135 | 0.6391137 | NA | 3.10E−107 | 2.47E−87 |
| Acad11 | NA | NA | 0.357135026 | NA | NA |
| Fn1 | 0.521230804 | 0.699634526 | 0.292522175 | 2.99E−26 | 1.87E−106 |
| Mia3 | NA | NA | 0.364623797 | NA | NA |
| Gm6484 | NA | NA | 0.304268201 | NA | NA |
| Zfp91 | NA | NA | 0.370686172 | NA | NA |
| Gm2a | NA | NA | 0.383109253 | NA | NA |
| Sec14l2 | −0.789737513 | NA | 0.321333578 | 5.48E−79 | NA |
| Cyp2a12 | NA | NA | 0.374332361 | NA | NA |
| Krt8 | 1.195874806 | NA | NA | 1.04E−103 | NA |
| Lpin2 | 0.686687426 | 0.277576437 | 0.385908326 | 6.95E−36 | 6.61E−21 |
| Clec4f | 1.108180782 | NA | NA | 2.17E−103 | NA |
| Cidec | 0.299384206 | NA | NA | 1.71E−102 | NA |
| B2m | −0.330354919 | −0.417973886 | NA | 5.62E−26 | 1.86E−102 |
| Slc4a4 | 0.420259653 | NA | 0.331670131 | 8.17E−17 | NA |
| Sh3glb1 | NA | NA | 0.326006747 | NA | NA |
| Tmed5 | 0.401633325 | NA | 0.345007803 | 1.17E−13 | NA |
| Serpinf1 | NA | NA | 0.319550979 | NA | NA |
| 0610005C13Rik | −0.425135941 | NA | 0.34237016 | 1.47E−21 | NA |
| Rpl4 | NA | NA | 0.283935593 | NA | NA |
| Dbi | 0.716365271 | NA | NA | 3.56E−101 | NA |
| Apoa4 | 1.367998503 | NA | 0.495579268 | 8.56E−101 | NA |
| Acot12 | NA | NA | 0.307840041 | NA | NA |
| Acot1 | −0.288638646 | −0.286204152 | 0.307840041 | 3.05E−10 | 5.23E−33 |
| Scp2 | −0.519124916 | NA | −0.325161752 | 1.05E−60 | NA |
| Uroc1 | NA | NA | 0.416226092 | NA | NA |
| Bcl2l1 | 0.775118224 | NA | NA | 1.02E−99 | NA |
| Mmp8 | 0.359005863 | NA | NA | 1.02E−99 | NA |
| Cyp2c29 | −1.149629979 | −0.586003377 | NA | 1.97E−99 | 2.74E−69 |
| Snord13 | NA | NA | 0.261078586 | NA | NA |
| Acadl | NA | NA | 0.316216015 | NA | NA |
| Cpt1a | 0.261119591 | NA | 0.372935212 | 0.000289401 | NA |
| Akr1d1 | −0.278865899 | NA | 0.361037667 | 6.11E−10 | NA |
| Cyp7b1 | −0.65802273 | NA | −0.542403458 | 4.96E−47 | NA |
| Mgll | NA | NA | 0.333899751 | NA | NA |
| Sult1d1 | −0.316458159 | NA | 0.45987026 | 4.92E−13 | NA |
| Slc25a10 | −0.282805644 | NA | 0.343272504 | 4.64E−10 | NA |
| Vnn3 | NA | NA | 0.276458974 | NA | NA |
| Pigr | 0.348711695 | NA | 0.386842112 | 8.87E−26 | NA |
| Pzp | NA | 0.41600896 | NA | NA | 3.34E−97 |
| Serpina1b | NA | −0.301770579 | NA | NA | 4.66E−97 |
| Tmem205 | −0.774742732 | −0.472107677 | NA | 1.21E−96 | 1.05E−96 |
| Mup14 | −0.697469805 | NA | −0.760766854 | 2.00E−25 | NA |
| Ces1b | NA | NA | 0.372958858 | NA | NA |
| C8g | −0.654510262 | −0.481964476 | NA | 3.81E−68 | 4.13E−96 |
| Cd14 | 0.362089625 | NA | NA | 2.79E−95 | NA |
| Mtdh | NA | NA | 0.30466184 | NA | NA |
| Hdlbp | 0.345721467 | 0.565147241 | NA | 5.40E−08 | 5.10E−95 |
| Tmbim4 | NA | NA | 0.32269634 | NA | NA |
| Saa2 | 0.558658405 | 0.964488054 | −0.390082382 | 0.000262015 | 8.96E−95 |
| Abcc3 | NA | NA | 0.363799323 | NA | NA |
| Serpina10 | NA | 0.565046404 | NA | NA | 8.21E−94 |
| Cyp2c44 | −0.372953076 | NA | 0.385935584 | 6.35E−18 | NA |
| Rcan1 | 0.707283787 | NA | NA | 1.24E−93 | NA |
| Arrdc3 | NA | 0.526871989 | NA | NA | 2.74E−93 |
| Hsd3b7 | −1.0059958 | −0.395256634 | −0.382738321 | 3.79E−93 | 2.93E−47 |
| Rell1 | 0.591181865 | NA | NA | 1.02E−92 | NA |
| Prdx1 | −0.529229113 | −0.267665106 | −0.361890861 | 7.29E−52 | 1.94E−38 |
| Fah | −0.259255187 | NA | 0.302494707 | 1.82E−10 | NA |
| Chpt1 | NA | NA | 0.35662056 | NA | NA |
| Mthfd2 | 0.262448025 | NA | NA | 1.46E−92 | NA |
| Gfra1 | 0.386382804 | NA | 0.320843264 | 8.44E−11 | NA |
| Retnlg | 0.375148336 | NA | NA | 4.49E−92 | NA |
| Pbld1 | −0.442542986 | NA | 0.322688203 | 7.81E−24 | NA |
| Creld2 | NA | 0.608652695 | NA | NA | 1.18E−91 |
| Aplp2 | NA | NA | 0.33938781 | NA | NA |
| Lifr | −0.626180925 | NA | −0.563681141 | 5.39E−42 | NA |
| Ces1d | NA | −0.428906017 | 0.285254637 | NA | 8.07E−91 |
| D10Wsu102e | 0.58780186 | NA | NA | 2.09E−90 | NA |
| Camk2n1 | NA | NA | 0.250109512 | NA | NA |
| Rps27l | −0.865927484 | −0.548506218 | NA | 1.39E−81 | 5.43E−90 |
| Bche | NA | NA | 0.313419985 | NA | NA |
| Itih3 | 0.546240791 | 0.492908293 | NA | 1.40E−47 | 1.11E−89 |
| Plscr1 | 0.444489505 | NA | NA | 2.65E−89 | NA |
| Gm24270 | NA | 0.26796863 | NA | NA | 3.13E−89 |
| Tmem56 | 0.273557142 | NA | 0.335238196 | 1 | NA |
| Rdx | NA | 0.373557306 | 0.318521875 | NA | 1.28E−35 |
| Abca6 | NA | NA | 0.33284944 | NA | NA |
| Gpld1 | NA | NA | 0.328106789 | NA | NA |
| Lrrc58 | 1.001096085 | NA | NA | 3.66E−88 | NA |
| Hba-a2 | 0.553546734 | NA | NA | 5.15E−88 | NA |
| Fxyd5 | 0.274279595 | NA | NA | 7.76E−88 | NA |
| Cyp8b1 | −0.889229825 | −0.553616206 | 0.282315072 | 6.32E−84 | 8.76E−88 |
| Cox6a1 | −0.48722865 | −0.366697192 | 0.292128773 | 1.69E−41 | 9.18E−60 |
| Ctage5 | NA | NA | 0.328678207 | NA | NA |
| Pex11a | NA | NA | 0.350827626 | NA | NA |
| Ass1 | NA | NA | −0.314400604 | NA | NA |
| Mttp | NA | NA | 0.311504107 | NA | NA |
| Eci2 | NA | NA | 0.311004843 | NA | NA |
| Prodh2 | NA | NA | 0.345294809 | NA | NA |
| Gadd45g | −0.355407686 | −0.614019025 | NA | 3.32E−18 | 7.38E−86 |
| Psen2 | −0.947373772 | NA | NA | 1.77E−85 | NA |
| Creg1 | NA | NA | 0.276773054 | NA | NA |
| BC025446 | NA | 0.29422883 | 0.272610935 | NA | 6.33E−31 |
| Serpina3n | 0.848313239 | 0.413696164 | 0.346482791 | 4.98E−61 | 4.35E−45 |
| Slc10a1 | −0.325757408 | NA | 0.314325805 | 3.37E−14 | NA |
| Met | NA | 0.288332662 | 0.283916114 | NA | 8.49E−25 |
| Mettl7b | −0.452656656 | NA | 0.283916114 | 2.76E−30 | NA |
| Pck1 | −0.652190949 | NA | 0.271859881 | 1.90E−84 | NA |
| Oat | −1.224853019 | −0.521209343 | NA | 6.25E−84 | 1.40E−70 |
| Pcbp2 | NA | NA | 0.310347013 | NA | NA |
| Atf5 | NA | NA | 0.329004772 | NA | NA |
| Tyrobp | 0.466551501 | NA | NA | 1.85E−83 | NA |
| C230081A13Rik | 0.426976229 | NA | NA | 3.42E−83 | NA |
| Hnf4a | 0.879108718 | 0.454905579 | 0.314710574 | 6.36E−71 | 9.91E−71 |
| Atxn7l3b | NA | NA | 0.306239775 | NA | NA |
| G0s2 | NA | NA | 0.346452213 | NA | NA |
| F10 | −0.271936054 | NA | 0.279095541 | 3.10E−11 | NA |
| Pla2g7 | 0.282344956 | NA | NA | 9.47E−83 | NA |
| Rplp0 | NA | NA | −0.430901519 | NA | NA |
| Gcdh | −0.300320098 | NA | 0.273385895 | 7.95E−15 | NA |
| Creb3l3 | NA | NA | 0.275700825 | NA | NA |
| Ssr3 | NA | 0.521769281 | NA | NA | 5.71E−82 |
| Eef1a1 | 0.282586942 | 0.439251983 | NA | 1.02E−09 | 2.20E−81 |
| Slc39a14 | 1.012749203 | 0.323040019 | NA | 2.25E−81 | 8.94E−22 |
| Man2a1 | 0.497115325 | 0.28327895 | 0.332755834 | 6.13E−22 | 2.79E−19 |
| Cox6c | −0.844280539 | −0.502802751 | NA | 3.24E−81 | 2.39E−77 |
| Il1r2 | 0.323447978 | NA | NA | 5.62E−81 | NA |
| Actn4 | 0.312388865 | NA | 0.299765947 | 1.98E−06 | NA |
| Ptprc | 0.416505491 | NA | NA | 1.46E−80 | NA |
| Eif5 | 0.576301866 | NA | 0.284340466 | 6.87E−46 | NA |
| Slc25a13 | NA | NA | 0.285473174 | NA | NA |
| Hint1 | −0.673880575 | −0.473665273 | NA | 4.75E−61 | 1.71E−80 |
| Echs1 | −0.331606352 | NA | 0.318692311 | 4.24E−19 | NA |
| Aco2 | NA | NA | 0.285855791 | NA | NA |
| Pnpla8 | NA | NA | 0.295631611 | NA | NA |
| C1qb | 0.681679654 | NA | NA | 1.02E−79 | NA |
| Hsd17b10 | 0.377206484 | NA | 0.361579378 | 4.44E−19 | NA |
| Egfr | NA | 0.504957782 | 0.473388687 | NA | 1.72E−79 |
| Cyp2a5 | −1.498910166 | −0.681077987 | NA | 3.74E−79 | 3.04E−57 |
| Asgr2 | NA | NA | 0.306445529 | NA | NA |
| Tmsb4x | 0.780616209 | NA | NA | 7.99E−79 | NA |
| Hsd17b13 | 0.680828119 | NA | 0.550399594 | 1.17E−50 | NA |
| Dennd5b | NA | 0.293458876 | 0.266493539 | NA | 1.07E−18 |
| Ctsh | NA | NA | 0.32445036 | NA | NA |
| Igf1 | NA | NA | 0.295791825 | NA | NA |
| Apc | 0.27896726 | 0.606730845 | NA | 7.91E−19 | 2.16E−78 |
| Apcs | 0.298925674 | 0.606730845 | NA | 0.75840106 | 2.16E−78 |
| Ctss | 0.764167083 | NA | NA | 2.26E−78 | NA |
| Cyp2d26 | NA | NA | 0.259136874 | NA | NA |
| Tcea3 | −0.355007815 | NA | 0.3163181 | 1.20E−14 | NA |
| Klf9 | NA | NA | 0.293415875 | NA | NA |
| Orm1 | NA | 0.365144434 | −0.31003929 | NA | 4.87E−78 |
| Clec4d | 0.26359638 | NA | NA | 5.98E−78 | NA |
| Mlycd | −0.290948387 | NA | 0.313046697 | 3.48E−09 | NA |
| Maob | NA | NA | 0.32037326 | NA | NA |
| Kif1b | 0.371764707 | NA | 0.299610278 | 0.000173425 | NA |
| Insig2 | 0.669991504 | NA | 0.300825179 | 1.24E−48 | NA |
| Ces3a | −0.52533635 | −0.36873562 | NA | 1.00E−62 | 5.80E−77 |
| G6pc | −0.960058274 | NA | NA | 1.00E−76 | NA |
| Cth | NA | −0.510899065 | NA | NA | 1.34E−76 |
| Pgrmc1 | NA | 0.350152674 | NA | NA | 1.59E−76 |
| Ugt2b1 | −0.776816931 | −0.289461704 | NA | 2.28E−76 | 4.20E−34 |
| Mup13 | −0.77574306 | −0.694889672 | −0.55255936 | 8.39E−43 | 2.45E−76 |
| Gabarap | −0.442546824 | NA | 0.289400057 | 2.22E−27 | NA |
| Arhgap5 | 0.264043476 | 0.295235575 | 0.288514066 | 0.057064443 | 2.61E−16 |
| Cyp1a2 | −0.993935836 | NA | −0.445766232 | 1.08E−75 | NA |
| Hgd | −0.758572265 | NA | NA | 1.96E−75 | NA |
| Adck3 | −0.87611538 | −0.28251534 | NA | 2.27E−75 | 6.63E−24 |
| Abhd14b | −0.39366782 | NA | 0.299363281 | 4.62E−20 | NA |
| Serinc3 | 0.35928911 | NA | 0.292320724 | 1.17E−06 | NA |
| Gltscr2 | NA | NA | 0.255310647 | NA | NA |
| Vcam1 | 0.457401491 | NA | NA | 6.00E−75 | NA |
| Urah | −0.764109132 | NA | NA | 1.55E−74 | NA |
| Sord | −0.383628337 | NA | 0.26850985 | 4.92E−29 | NA |
| Agpat9 | 0.327040595 | NA | NA | 2.14E−74 | NA |
| Etnppl | 0.41816318 | −0.428291487 | 0.496712405 | 2.26E−13 | 4.23E−36 |
| Por | 0.335963246 | −0.271635533 | 0.33194923 | 4.49E−09 | 2.42E−08 |
| Rsp14 | −0.402093042 | −0.292251029 | −0.348405505 | 1.50E−30 | 3.26E−44 |
| Lamp1 | NA | NA | 0.268283663 | NA | NA |
| Plek | 0.308857869 | NA | NA | 1.40E−73 | NA |
| Ctsa | NA | NA | 0.252009224 | NA | NA |
| Mrap | 0.884099926 | NA | NA | 2.65E−73 | NA |
| Dnajc3 | NA | 0.45638751 | NA | NA | 2.66E−73 |
| Adamts1 | 0.282556845 | NA | NA | 8.88E−73 | NA |
| Hadh | NA | NA | 0.288772268 | NA | NA |
| C8b | −0.315917765 | 0.318703124 | −0.360171247 | 1.33E−15 | 1.74E−50 |
| Csad | −0.559786018 | −0.531252493 | NA | 1.25E−34 | 1.92E−72 |
| Nr1d2 | NA | 0.430636791 | NA | NA | 2.60E−72 |
| Sod1 | −0.439319795 | −0.303629074 | NA | 7.55E−60 | 3.30E−72 |
| Ech1 | NA | −0.429132615 | 0.250481301 | NA | 5.62E−72 |
| Ddb1 | NA | NA | 0.277809186 | NA | NA |
| Kng1 | NA | 0.265777676 | NA | NA | 1.32E−71 |
| Shmt1 | −0.393237106 | NA | 0.278820727 | 1.10E−18 | NA |
| Acsl1 | −0.343424014 | −0.370316988 | 0.256335416 | 2.16E−20 | 1.40E−54 |
| C4b | NA | NA | 0.283273876 | NA | NA |
| Gcnt2 | 0.537509676 | NA | NA | 4.50E−70 | NA |
| Csf1r | 0.704458586 | NA | NA | 4.70E−70 | NA |
| Ggnbp2 | NA | NA | 0.272604502 | NA | NA |
| Nfe2l1 | NA | NA | 0.27034473 | NA | NA |
| Tomm70a | NA | 0.268786029 | 0.265762726 | NA | 3.18E−17 |
| Steap4 | 0.437231109 | 0.475973731 | NA | 3.54E−11 | 6.59E−69 |
| Atp2a2 | 0.494962728 | NA | 0.305636215 | 2.90E−18 | NA |
| Cd1d1 | NA | NA | 0.291156805 | NA | NA |
| Comt | −0.368377448 | NA | −0.286248465 | 2.17E−28 | NA |
| Sec62 | NA | NA | 0.251575572 | NA | NA |
| Cxcr2 | 0.40229963 | NA | NA | 2.86E−68 | NA |
| Slc7a2 | NA | 0.352564931 | 0.34497807 | NA | 1.75E−31 |
| C130074G19Rik | −0.356338948 | NA | 0.250322698 | 8.18E−13 | NA |
| Apob | 0.580156235 | 0.4272457 | NA | 5.69E−61 | 4.36E−68 |
| Furin | 0.253587917 | NA | 0.278163716 | 0.405633752 | NA |
| Cxcl1 | 0.370070418 | 0.449766784 | 0.257250112 | 4.89E−08 | 1.26E−30 |
| Cxcl12 | −0.305838679 | NA | 0.257250112 | 6.12E−10 | NA |
| Khk | −0.491453217 | −0.29761703 | 0.310103866 | 3.36E−37 | 4.03E−35 |
| Ei24 | NA | NA | 0.257327011 | NA | NA |
| Cd163 | 0.440845529 | NA | NA | 1.51E−67 | NA |
| Trp53inp1 | 1.008414088 | 0.367960906 | NA | 1.52E−67 | 2.95E−29 |
| Eif4g2 | 0.559692663 | 0.465990267 | NA | 5.94E−33 | 1.62E−67 |
| Gys2 | NA | NA | 0.274401611 | NA | NA |
| Dmgdh | NA | NA | 0.265846058 | NA | NA |
| Tob1 | −0.338705886 | 0.312925608 | 0.284985634 | 9.48E−15 | 1.91E−21 |
| Ubc | −0.661839716 | −0.370487607 | NA | 4.04E−67 | 2.42E−49 |
| Cbr1 | 0.284717454 | NA | 0.260731659 | 7.95E−05 | NA |
| Pon1 | −0.685751275 | −0.322748466 | NA | 1.66E−66 | 5.87E−44 |
| Suds3 | NA | NA | 0.25388963 | NA | NA |
| 1700017B05Rik | 0.330656885 | NA | NA | 2.40E−66 | NA |
| Ugp2 | NA | NA | 0.297335445 | NA | NA |
| Iigp1 | 0.768753667 | 0.454178892 | NA | 2.65E−29 | 7.59E−66 |
| Nop58 | 0.663449653 | NA | NA | 2.13E−65 | NA |
| Padi3 | 0.295682522 | NA | NA | 2.63E−65 | NA |
| Pipox | NA | NA | 0.272543042 | NA | NA |
| Ndufa4 | −0.683126695 | −0.443311137 | NA | 1.11E−61 | 3.70E−65 |
| Gsta1 | 0.407606856 | NA | NA | 7.64E−65 | NA |
| F9 | NA | NA | 0.256805166 | NA | NA |
| B4galt5 | 0.476306558 | NA | NA | 1.12E−64 | NA |
| F12 | NA | NA | 0.256737037 | NA | NA |
| Fcgrt | NA | −0.269314211 | 0.258478842 | NA | 8.21E−19 |
| Fmo5 | 0.286219883 | NA | 0.252070931 | 1.05E−08 | NA |
| Rnf144a | 0.313046134 | NA | NA | 7.90E−64 | NA |
| Ugt2b36 | −0.583431487 | −0.329316239 | NA | 1.36E−63 | 1.07E−57 |
| Sardh | NA | NA | 0.269183147 | NA | NA |
| Iqgap1 | 0.45544473 | NA | NA | 1.82E−63 | NA |
| Creb3l2 | 0.427180051 | NA | NA | 1.91E−63 | NA |
| Slc22a1 | −0.797941816 | NA | NA | 2.27E−63 | NA |
| Lyz2 | 0.732183369 | NA | NA | 2.49E−63 | NA |
| Bach1 | 0.832333098 | NA | NA | 2.51E−63 | NA |
| Lpgat1 | 0.595219291 | 0.394625955 | 0.257600415 | 4.45E−21 | 3.86E−28 |
| Atp1a1 | 0.419079339 | 0.270031448 | 0.262770413 | 2.42E−07 | 1.97E−06 |
| Fam134b | 0.893562894 | NA | NA | 5.02E−63 | NA |
| Hsd17b4 | NA | NA | 0.253232967 | NA | NA |
| Gabarapl1 | NA | NA | 0.260716513 | NA | NA |
| Cd5l | 0.693485826 | NA | NA | 1.08E−62 | NA |
| Elovl2 | NA | 0.45224665 | NA | NA | 1.22E−62 |
| Lrg1 | 0.490792829 | 0.436838294 | NA | 1.92E−28 | 1.37E−62 |
| Hgfac | NA | NA | 0.252189991 | NA | NA |
| Atp5d | NA | NA | 0.250062798 | NA | NA |
| Efhd2 | 0.583831769 | NA | NA | 3.26E−62 | NA |
| Pcbd1 | −0.687761883 | NA | NA | 6.29E−62 | NA |
| mmu-mir-6240 | 1.101237189 | NA | NA | 9.82E−62 | NA |
| Hagh | −0.674009711 | NA | NA | 1.62E−61 | NA |
| Hsd3b3 | −0.779501123 | NA | −0.299662144 | 1.98E−61 | NA |
| Tmem106b | 0.280204492 | NA | 0.270357032 | 0.106548775 | NA |
| Ctsd | NA | NA | 0.257862212 | NA | NA |
| Cd44 | 0.303745221 | NA | NA | 5.24E−61 | NA |
| Rdh16 | NA | NA | 0.269182372 | NA | NA |
| Cyp2c70 | −0.551134023 | 0.336400438 | −0.395158527 | 4.62E−37 | 2.87E−40 |
| Thrsp | −0.724961807 | NA | −0.689057582 | 3.13E−30 | NA |
| Hadha | NA | NA | 0.250937029 | NA | NA |
| Sdhd | −0.335261252 | NA | 0.320841923 | 2.07E−18 | NA |
| Mbl1 | −0.713006005 | NA | NA | 1.05E−60 | NA |
| Ddt | −0.522221147 | −0.422410948 | NA | 1.48E−41 | 1.33E−60 |
| Eif1a | 0.795281298 | NA | NA | 1.80E−60 | NA |
| Alb | 0.420298986 | NA | NA | 1.86E−60 | NA |
| Pdia4 | −0.27191836 | 0.488166898 | NA | 8.81E−08 | 1.94E−60 |
| F5 | NA | NA | 0.300566587 | NA | NA |
| Laptm5 | 0.338180771 | NA | NA | 3.31E−60 | NA |
| Slc2a2 | −0.312348392 | NA | 0.272297971 | 2.36E−14 | NA |
| Sfxn1 | NA | NA | 0.304885886 | NA | NA |
| Epas1 | 0.775763153 | NA | NA | 7.57E−60 | NA |
| Alas1 | NA | −0.489837477 | NA | NA | 8.49E−60 |
| Cd97 | 0.345774315 | NA | NA | 1.02E−59 | NA |
| Hacl1 | −0.706897734 | NA | NA | 1.20E−59 | NA |
| Oaz1 | −0.347228062 | −0.374797984 | −0.418528822 | 1.73E−17 | 8.14E−43 |
| Hpd | −0.518180386 | −0.313324027 | NA | 2.47E−59 | 1.72E−56 |
| Cd74 | 0.453937562 | NA | NA | 3.15E−59 | NA |
| Aldh3a2 | −0.834229334 | −0.300776093 | 0.307877353 | 3.38E−59 | 3.40E−27 |
| Gclm | 0.269864161 | NA | 0.258765469 | 2.58E−05 | NA |
| Serpina3c | −0.287011328 | −0.408878947 | −0.472511045 | 7.83E−06 | 1.55E−34 |
| Sdc4 | 0.619597869 | NA | NA | 9.47E−59 | NA |
| Ubxn4 | NA | NA | 0.250313647 | NA | NA |
| Ptms | NA | NA | 0.267838974 | NA | NA |
| Wfdc17 | 0.486653465 | NA | NA | 2.57E−58 | NA |
| Gsta2 | NA | NA | 0.252161786 | NA | NA |
| 1110008F13Rik | −0.37509166 | −0.455270589 | NA | 2.46E−19 | 4.13E−58 |
| Fkbp5 | 0.516128063 | NA | NA | 1.44E−57 | NA |
| mt-Nd6 | NA | −0.477851134 | NA | NA | 1.67E−57 |
| Kctd3 | 0.260833465 | NA | NA | 2.11E−57 | NA |
| Sec61a1 | NA | 0.313944633 | 0.263729835 | NA | 2.96E−24 |
| Ddx21 | 0.73138069 | NA | NA | 2.25E−57 | NA |
| Pdcd4 | NA | 0.48815423 | NA | NA | 3.64E−57 |
| Upb1 | −0.418695518 | NA | 0.25038758 | 3.32E−25 | NA |
| Cyp2c37 | −0.886903784 | −0.487522606 | NA | 9.93E−57 | 9.75E−56 |
| Cd53 | 0.265579528 | NA | NA | 1.37E−56 | NA |
| Park7 | −0.260230015 | NA | 0.251517689 | 5.75E−09 | NA |
| Hic2 | 0.289767391 | NA | NA | 2.03E−56 | NA |
| Sat1 | 0.68869896 | NA | NA | 2.31E−56 | NA |
| Btg2 | 0.832086378 | NA | NA | 3.81E−56 | NA |
| Usmg5 | −0.43049483 | NA | 0.278730923 | 2.85E−18 | NA |
| Rgs2 | 0.395729601 | NA | NA | 7.50E−56 | NA |
| Chchd2 | −0.552503492 | −0.394916772 | NA | 2.39E−48 | 7.88E−56 |
| Slc37a4 | NA | NA | 0.268318739 | NA | NA |
| Inmt | −0.731016404 | −0.44352952 | −0.25322177 | 1.21E−55 | 1.57E−51 |
| Cyp2d10 | NA | −0.323151441 | NA | NA | 1.31E−55 |
| Itgb2 | 0.272302012 | NA | NA | 1.36E−55 | NA |
| Cps1 | NA | 0.32450205 | NA | NA | 1.45E−55 |
| Keg1 | −0.824548137 | NA | −0.521611435 | 1.46E−55 | NA |
| C1qc | 0.586092716 | NA | NA | 2.05E−55 | NA |
| AI182371 | −0.319894937 | 0.447854829 | NA | 9.02E−17 | 2.62E−55 |
| Rock2 | 0.594094466 | NA | NA | 2.90E−55 | NA |
| Sqstm1 | 0.743150456 | NA | NA | 4.59E−55 | NA |
| Fndc3b | 0.409645989 | 0.348474602 | NA | 1.32E−37 | 4.69E−55 |
| Uox | −0.553115511 | NA | NA | 6.13E−55 | NA |
| Gulo | −0.801665727 | NA | NA | 6.80E−55 | NA |
| mt-Tq | NA | 0.287889985 | NA | NA | 7.13E−55 |
| C8a | NA | 0.333650228 | −0.327131388 | NA | 3.84E−39 |
| Tiparp | 0.545567123 | NA | NA | 8.73E−55 | NA |
| Fcgr3 | 0.308247077 | NA | NA | 1.21E−54 | NA |
| Uqcr11 | −0.603079991 | −0.263241396 | NA | 1.37E−54 | 5.87E−27 |
| Atp5e | −0.650247857 | −0.313641686 | NA | 2.15E−54 | 1.78E−29 |
| Tram1 | NA | 0.411080257 | NA | NA | 2.39E−54 |
| Prg4 | NA | 0.430643187 | NA | NA | 3.20E−54 |
| Upp2 | −0.69025963 | NA | NA | 3.29E−54 | NA |
| H2-Ab1 | 0.460956687 | NA | NA | 3.42E−54 | NA |
| Gm26917 | 0.60120474 | 0.823736053 | NA | 6.50E−24 | 5.26E−54 |
| Aldh8a1 | −0.683676902 | NA | NA | 5.66E−54 | NA |
| Glyat | −0.647687911 | NA | NA | 1.11E−53 | NA |
| Msrb1 | −0.59903268 | NA | NA | 1.16E−53 | NA |
| Manf | NA | 0.468881688 | NA | NA | 1.60E−53 |
| Spred1 | 0.388422711 | NA | NA | 1.65E−53 | NA |
| Orm2 | NA | 0.271460545 | NA | NA | 2.71E−53 |
| Sparcl1 | −0.253616986 | −0.253616986 | −0.253616986 | 2.56E−11 | 1.01E−31 |
| Cat | NA | 0.262624418 | NA | NA | 7.22E−53 |
| Ndufb11 | −0.509671057 | −0.42752525 | −0.275645983 | 3.19E−34 | 8.28E−53 |
| C1qa | 0.514846404 | NA | NA | 1.51E−52 | NA |
| Cldn14 | 0.364540783 | NA | NA | 1.86E−52 | NA |
| Heatr1 | 0.406005371 | NA | NA | 2.23E−52 | NA |
| Calm1 | NA | 0.413146039 | NA | NA | 2.28E−52 |
| Insig1 | NA | 0.465534816 | NA | NA | 3.03E−52 |
| Cfh | NA | 0.400919606 | −0.306325678 | NA | 3.24E−52 |
| Ppp2r5a | −0.260504538 | NA | 0.250897706 | 9.42E−10 | NA |
| Hc | NA | 0.367935082 | NA | NA | 1.11E−51 |
| Hebp1 | −0.635432267 | NA | NA | 1.18E−51 | NA |
| Ces1e | NA | NA | 0.265618893 | NA | NA |
| Cox7b | −0.642240776 | −0.267397469 | NA | 3.42E−51 | 4.55E−23 |
| Abhd2 | 0.484366625 | NA | NA | 1.13E−50 | NA |
| Picalm | 0.754529703 | NA | NA | 1.39E−50 | NA |
| Igfbp1 | 1.058689301 | −0.316195447 | NA | 1.68E−50 | 9.05E−32 |
| Haao | −0.591181029 | NA | NA | 2.08E−50 | NA |
| Canx | NA | 0.343383207 | NA | NA | 2.09E−50 |
| Cebpb | −0.361738286 | −0.471579534 | NA | 9.79E−11 | 2.38E−50 |
| Stat3 | 0.715400235 | 0.297428438 | NA | 2.74E−50 | 8.01E−18 |
| Fgl2 | 0.281793883 | NA | NA | 2.86E−50 | NA |
| AA474408 | 0.347079511 | 0.43640813 | NA | 8.58E−25 | 3.01E−50 |
| Lyst | 0.409280587 | NA | NA | 8.39E−50 | NA |
| Myo1e | 0.614341001 | NA | NA | 9.34E−50 | NA |
| Tmem87b | 0.434399269 | NA | NA | 1.07E−49 | NA |
| Slc10a2 | 0.299006165 | NA | NA | 2.04E−49 | NA |
| Gnl3 | 0.575970422 | NA | NA | 2.63E−49 | NA |
| Lcn2 | 0.412194792 | NA | NA | 2.72E−49 | NA |
| Jmjd1c | 0.636619821 | NA | NA | 3.43E−49 | NA |
| Cox7a2 | −0.650338386 | −0.363458039 | NA | 2.00E−48 | 5.69E−35 |
| Cltc | 0.784985399 | 0.438067775 | NA | 3.05E−48 | 2.75E−44 |
| Rarres2 | −0.552194576 | −0.266616471 | NA | 3.29E−48 | 1.17E−31 |
| Sc5d | −0.500900917 | 0.345016882 | NA | 2.09E−27 | 3.57E−48 |
| Ndufa6 | −0.613649978 | −0.306815302 | NA | 4.43E−48 | 4.71E−27 |
| 2-Mar | 0.55994977 | NA | NA | 4.66E−48 | NA |
| Hspa13 | NA | 0.354844877 | NA | NA | 7.52E−48 |
| Btg1 | 0.458007046 | NA | NA | 1.02E−47 | NA |
| Pah | −0.52207695 | NA | NA | 1.25E−47 | NA |
| Leap2 | −0.671300937 | −0.274778129 | NA | 1.25E−47 | 1.45E−22 |
| Cytip | 0.267599208 | NA | NA | 1.05E−46 | NA |
| Clpx | NA | −0.404735614 | NA | NA | 1.07E−46 |
| Sdc3 | 0.359804895 | NA | NA | 1.11E−46 | NA |
| mt-Tm | NA | 0.284684642 | NA | NA | 1.20E−46 |
| Gm24187 | 0.431940663 | NA | NA | 1.37E−46 | NA |
| Stk40 | 0.536418312 | NA | NA | 2.15E−46 | NA |
| Cyp2c50 | −0.661823297 | −0.290483498 | NA | 2.92E−46 | 1.48E−24 |
| Cfp | 0.356178244 | NA | NA | 6.11E−46 | NA |
| Dot1l | 0.397036294 | NA | NA | 7.81E−46 | NA |
| Saa4 | −0.571021905 | 0.375528565 | NA | 2.31E−40 | 1.16E−45 |
| Atp8b1 | NA | 0.362807422 | NA | NA | 1.42E−45 |
| Mdn1 | 0.317989246 | NA | NA | 1.53E−45 | NA |
| Ndufb2 | −0.577154458 | −0.341226284 | 0.251595204 | 5.29E−40 | 2.52E−28 |
| Hsd17b2 | −0.595878526 | NA | −0.313480586 | 3.18E−45 | NA |
| H2-Eb1 | 0.327872672 | NA | NA | 3.30E−45 | NA |
| Cxadr | 0.698450944 | NA | NA | 4.99E−45 | NA |
| Gstm3 | −0.411092337 | −0.318590137 | −0.598573335 | 1.22E−06 | 2.69E−10 |
| Cpb2 | −0.321495225 | 0.322179327 | NA | 4.76E−14 | 5.47E−45 |
| Spcs3 | NA | 0.394206972 | NA | NA | 9.16E−45 |
| Cd302 | −0.549925078 | NA | NA | 1.10E−44 | NA |
| Plg | NA | 0.261736006 | NA | NA | 1.34E−44 |
| Lactb2 | −0.596505052 | NA | NA | 1.72E−44 | NA |
| Tsr1 | 0.360287403 | NA | NA | 2.32E−44 | NA |
| Igfbp2 | −0.298981027 | −0.345087789 | NA | 1.25E−14 | 4.02E−44 |
| Rps2 | −0.387862655 | −0.384454173 | −0.374246561 | 2.13E−20 | 6.76E−40 |
| Dgkd | 0.370844881 | NA | NA | 1.62E−43 | NA |
| Cml1 | −0.61424052 | −0.326909468 | NA | 1.70E−43 | 2.10E−29 |
| Sorl1 | 0.306655621 | NA | NA | 1.87E−43 | NA |
| Tra2a | 0.712328169 | NA | NA | 2.35E−43 | NA |
| Dcxr | −0.440851615 | −0.410666503 | NA | 2.61E−26 | 2.61E−43 |
| Aadac | −0.619958028 | −0.40376663 | NA | 4.18E−43 | 5.65E−43 |
| Ppib | −0.589699514 | NA | −0.260118322 | 1.14E−42 | NA |
| Lect2 | −0.626967771 | NA | NA | 1.41E−42 | NA |
| Tlcd2 | −0.594498821 | NA | NA | 1.60E−42 | NA |
| Lpin1 | NA | NA | 0.277494868 | NA | NA |
| Ttc39c | −0.636274928 | NA | −0.374504475 | 2.03E−42 | NA |
| Tacc2 | 0.329332932 | NA | NA | 3.95E−42 | NA |
| Gchfr | −0.554783473 | −0.386867237 | NA | 5.12E−39 | 4.46E−42 |
| Tmed7 | NA | 0.386771014 | NA | NA | 6.68E−42 |
| Usp9x | 0.318385696 | 0.407617259 | NA | 2.35E−09 | 9.98E−42 |
| Aldh1a1 | −0.478797447 | NA | NA | 1.02E−41 | NA |
| Sdad1 | 0.312421192 | NA | NA | 1.30E−41 | NA |
| Fdx1 | −0.623439092 | −0.344715083 | NA | 1.55E−41 | 4.65E−26 |
| Eef2 | 0.525855561 | 0.270115373 | NA | 2.71E−41 | 2.54E−28 |
| Gstz1 | −0.464086834 | NA | NA | 2.81E−41 | NA |
| Ptpn1 | 0.41633121 | NA | NA | 3.51E−41 | NA |
| Tnfrsf1a | 0.599573483 | NA | NA | 3.86E−41 | NA |
| Rad54l2 | 0.388705156 | NA | NA | 4.52E−41 | NA |
| Il13ra1 | 0.278527191 | 0.30296595 | NA | 6.54E−12 | 4.97E−41 |
| Pemt | −0.551184095 | NA | NA | 7.86E−41 | NA |
| Acss2 | −0.620071374 | NA | −0.258362678 | 8.55E−41 | NA |
| 1-Mar | −0.477762617 | NA | NA | 9.43E−41 | NA |
| Ddx3x | 0.656890035 | NA | NA | 1.07E−40 | NA |
| Hs3st3b1 | NA | 0.343007923 | NA | NA | 1.60E−40 |
| Acox1 | NA | 0.275171428 | NA | NA | 1.78E−40 |
| Atp5j2 | −0.51308051 | NA | NA | 3.32E−40 | NA |
| 8430408G22Rik | −0.50210224 | NA | NA | 4.85E−40 | NA |
| Ndufb9 | −0.503828812 | NA | NA | 5.48E−40 | NA |
| Hif1a | 0.516911277 | NA | NA | 5.71E−40 | NA |
| Hes6 | −0.561302344 | NA | −0.262263942 | 5.81E−40 | NA |
| Tsku | 0.36762763 | NA | NA | 6.05E−40 | NA |
| Dnajc12 | NA | 0.322094554 | NA | NA | 1.14E−39 |
| Eaf1 | 0.521796522 | NA | NA | 1.19E−39 | NA |
| Qdpr | −0.52357376 | NA | NA | 1.45E−39 | NA |
| Atp5f1 | −0.517061165 | −0.28724025 | NA | 2.05E−39 | 1.01E−28 |
| Gm4076 | NA | −0.325469418 | −0.308321155 | NA | 1.37E−35 |
| Uso1 | NA | 0.388069473 | NA | NA | 3.58E−39 |
| Maff | 0.355300257 | NA | NA | 4.89E−39 | NA |
| Qsox1 | NA | 0.342352868 | NA | NA | 9.20E−39 |
| Adh4 | −0.526868306 | 0.376928101 | NA | 1.92E−38 | 1.20E−30 |
| Chic2 | 0.419790279 | NA | NA | 2.33E−38 | NA |
| Eef1b2 | NA | NA | −0.317323205 | NA | NA |
| Adh5 | −0.491433382 | NA | NA | 2.88E−38 | NA |
| Itih2 | 0.501091194 | 0.298659728 | NA | 3.43E−38 | 6.17E−35 |
| Gjb2 | 0.60822545 | 0.391136188 | NA | 1.12E−27 | 3.74E−38 |
| Fabp2 | −0.615569339 | −0.39219613 | NA | 4.18E−38 | 3.19E−36 |
| Slc20a1 | 0.357248475 | NA | NA | 6.66E−38 | NA |
| Slc3a1 | NA | 0.340997565 | NA | NA | 7.10E−38 |
| Mup2 | −0.50447051 | −0.274243623 | −0.55305847 | 9.59E−13 | 1 |
| Abca1 | 0.724477701 | 0.439378423 | NA | 8.81E−38 | 4.14E−37 |
| Ndufa7 | −0.555912274 | NA | NA | 1.32E−37 | NA |
| Lgmn | 0.484586871 | NA | NA | 1.80E−37 | NA |
| Gamt | −0.560282685 | NA | NA | 2.41E−37 | NA |
| Bcl3 | 0.289017645 | NA | NA | 3.63E−37 | NA |
| Hint2 | −0.424321102 | −0.37016853 | NA | 5.24E−24 | 6.90E−37 |
| Mpc2 | −0.482839417 | NA | NA | 7.20E−37 | NA |
| Atxn1 | NA | 0.304881734 | NA | NA | 9.14E−37 |
| Cyp2c54 | −0.39493764 | −0.326859698 | 0.250387024 | 1.78E−20 | 3.25E−32 |
| Fos | NA | 0.432003299 | NA | NA | 1.43E−36 |
| St3gal1 | 0.528739882 | NA | NA | 1.81E−36 | NA |
| Msn | 0.260172491 | NA | NA | 1.86E−36 | NA |
| Sds | 0.390741627 | −0.407006722 | 0.297273523 | 7.29E−12 | 2.82E−36 |
| Rpn1 | NA | 0.41242919 | NA | NA | 5.16E−36 |
| Ucp2 | 0.261164666 | NA | NA | 8.74E−36 | NA |
| Tcerg1 | 0.357549618 | NA | NA | 1.14E−35 | NA |
| Akr1c20 | −0.554152364 | NA | NA | 1.48E−35 | NA |
| Dst | 0.510971252 | 0.324301394 | NA | 2.08E−26 | 1.75E−35 |
| Dstn | NA | 0.324301394 | NA | NA | 1.75E−35 |
| S100a1 | −0.374338118 | −0.376186192 | NA | 1.68E−18 | 1.76E−35 |
| Sar1b | −0.307930709 | −0.317607513 | NA | 2.07E−14 | 1.79E−35 |
| Crp | −0.506869364 | NA | NA | 2.22E−35 | NA |
| Pfkfb2 | 0.290016291 | NA | NA | 2.41E−35 | NA |
| Gm4952 | −0.49867829 | NA | NA | 4.03E−35 | NA |
| Ndufb6 | −0.507682563 | −0.306403298 | NA | 4.30E−35 | 4.45E−28 |
| Vsig4 | 0.483290635 | NA | NA | 4.48E−35 | NA |
| Hao1 | NA | 0.32303248 | NA | NA | 4.98E−35 |
| Tubb2a | 0.749433744 | NA | NA | 5.17E−35 | NA |
| Arl4d | −0.507228317 | NA | NA | 6.71E−35 | NA |
| mmu-mir-6236 | 0.520569164 | 0.787474192 | −0.429737333 | 2.18E−05 | 7.24E−35 |
| Apobec1 | 0.305082858 | NA | NA | 9.49E−35 | NA |
| Akr1c14 | −0.518570798 | NA | NA | 1.08E−34 | NA |
| Tmem30a | NA | 0.332760453 | NA | NA | 1.95E−34 |
| Cfhr2 | −0.44846316 | −0.321928662 | −0.306325678 | 4.83E−25 | 4.87E−29 |
| Slc41a2 | 0.260152635 | 0.278262888 | NA | 6.42E−16 | 2.13E−34 |
| Ipo7 | 0.324580152 | 0.25915239 | NA | 1.14E−13 | 2.19E−34 |
| Tsc22d2 | 0.391059638 | NA | NA | 2.43E−34 | NA |
| Cyb5r3 | NA | 0.339033056 | NA | NA | 3.10E−34 |
| Apol7a | −0.492294435 | NA | NA | 3.11E−34 | NA |
| Fxyd1 | −0.479908398 | −0.311759144 | NA | 2.89E−33 | 3.83E−34 |
| Cox5b | −0.498783616 | −0.337825031 | NA | 4.68E−34 | 1.67E−32 |
| Rplp1 | −0.504662964 | −0.286640389 | NA | 4.91E−34 | 5.94E−23 |
| Ugdh | 0.633076584 | NA | NA | 5.17E−34 | NA |
| H2-Aa | 0.327934784 | NA | NA | 6.84E−34 | NA |
| H3f3b | 0.702914634 | NA | NA | 7.03E−34 | NA |
| Ppan | 0.272410087 | NA | NA | 7.48E−34 | NA |
| Ugt2a3 | −0.307528085 | −0.333441405 | NA | 3.75E−16 | 8.21E−34 |
| Xbp1 | 0.603165814 | NA | NA | 9.59E−34 | NA |
| Tjp2 | 0.411461753 | NA | NA | 1.10E−33 | NA |
| Tatdn2 | 0.373296072 | NA | NA | 1.22E−33 | NA |
| Hibadh | −0.499409672 | NA | NA | 1.26E−33 | NA |
| Pten | NA | 0.355902258 | NA | NA | 1.92E−33 |
| Ctsl | 0.530101805 | NA | NA | 1.93E−33 | NA |
| Hspb8 | 0.633933124 | NA | NA | 2.00E−33 | NA |
| Atp5j | −0.48373528 | NA | NA | 3.59E−33 | NA |
| Usp4 | 0.279964381 | 0.324732042 | NA | 8.83E−09 | 4.02E−33 |
| Nolc1 | 0.47222269 | NA | NA | 4.11E−33 | NA |
| Ier5 | 0.288994609 | NA | NA | 4.64E−33 | NA |
| Lonp2 | −0.467365882 | NA | NA | 5.23E−33 | NA |
| Rpl32 | −0.532218378 | −0.254910119 | −0.284689184 | 1.10E−32 | 2.90E−16 |
| Rrbp1 | NA | 0.306944991 | NA | NA | 1.11E−32 |
| Dio1 | −0.545283744 | NA | NA | 1.11E−32 | NA |
| Tmem256 | −0.518510202 | NA | NA | 1.19E−32 | NA |
| Pdia6 | −0.389966782 | 0.336506977 | NA | 5.65E−20 | 1.21E−32 |
| Ssr1 | NA | 0.327818552 | NA | NA | 1.30E−32 |
| Gm5096 | NA | −0.363089147 | NA | NA | 1.95E−32 |
| Mcm10 | −0.487427572 | NA | NA | 2.29E−32 | NA |
| Ibtk | NA | 0.331106132 | NA | NA | 4.10E−32 |
| Nit2 | −0.485940613 | NA | NA | 4.49E−32 | NA |
| Ppm1k | −0.558394496 | NA | NA | 6.56E−32 | NA |
| Son | 0.542911509 | NA | NA | 7.13E−32 | NA |
| Adh6-ps1 | −0.418504072 | NA | NA | 9.38E−32 | NA |
| Selenbp1 | −0.493707332 | −0.280021704 | NA | 9.52E−32 | 1.67E−21 |
| Uggt1 | NA | 0.284457726 | NA | NA | 1.12E−31 |
| Efna1 | NA | 0.336097237 | NA | NA | 1.73E−31 |
| Ssr4 | −0.507577243 | NA | NA | 1.87E−31 | NA |
| Ndufs6 | −0.537929827 | −0.359160953 | NA | 2.00E−31 | 4.72E−31 |
| 1600014C10Rik | −0.498182953 | NA | NA | 2.43E−31 | NA |
| Gas5 | NA | −0.336153154 | NA | NA | 2.74E−31 |
| Nop56 | 0.265670844 | NA | NA | 2.87E−31 | NA |
| Etfb | −0.365490277 | NA | NA | 4.01E−31 | NA |
| Dusp16 | 0.46051738 | NA | NA | 4.23E−31 | NA |
| Rora | 0.644354953 | NA | NA | 5.02E−31 | NA |
| Nedd4 | 0.281232817 | 0.377388355 | NA | 4.90E−18 | 6.69E−31 |
| Epb4.1 | 0.562789274 | NA | NA | 1.67E−30 | NA |
| Csde1 | 0.315554781 | 0.326883793 | NA | 2.59E−06 | 1.68E−30 |
| Ranbp2 | 0.598499679 | 0.26594209 | NA | 2.18E−30 | 6.71E−19 |
| Kmo | −0.384440188 | 0.302208311 | NA | 6.51E−22 | 3.26E−30 |
| Ndufa13 | −0.43876131 | NA | NA | 3.68E−30 | NA |
| Rnd3 | 0.36548808 | NA | NA | 3.93E−30 | NA |
| Prkd3 | NA | 0.366517526 | NA | NA | 3.93E−30 |
| Nr5a2 | NA | 0.322039014 | NA | NA | 5.05E−30 |
| Gm23388 | 0.416208382 | NA | NA | 5.50E−30 | NA |
| Ddx5 | 0.522944475 | NA | NA | 5.90E−30 | NA |
| Blvrb | −0.545112721 | NA | NA | 6.23E−30 | NA |
| Lrp1 | NA | 0.505128824 | NA | NA | 6.85E−30 |
| Cfi | NA | 0.251606433 | NA | NA | 7.20E−30 |
| Mmp14 | 0.288913135 | NA | NA | 7.66E−30 | NA |
| Plec | 0.467512335 | NA | NA | 8.30E−30 | NA |
| Rab1 | NA | 0.352627435 | NA | NA | 1.03E−29 |
| Jak1 | 0.603850485 | 0.254122657 | NA | 1.34E−29 | 1.20E−17 |
| Slco1a1 | −0.362843757 | NA | −0.295640242 | 2.16E−16 | NA |
| Errfi1 | 0.393680047 | NA | NA | 1.52E−29 | NA |
| Slc38a4 | NA | 0.263698621 | NA | NA | 2.48E−29 |
| Uqcr10 | −0.45190185 | NA | NA | 2.60E−29 | NA |
| Nab1 | NA | 0.33421071 | NA | NA | 3.00E−29 |
| Il6st | 0.316786986 | 0.333486533 | NA | 1.69E−06 | 3.02E−29 |
| Mpc1 | −0.280163495 | −0.281762702 | NA | 1.13E−10 | 3.39E−29 |
| Cmah | NA | 0.361552447 | NA | NA | 5.19E−29 |
| Calu | NA | 0.304112196 | NA | NA | 5.69E−29 |
| Mlec | NA | 0.324558531 | NA | NA | 7.94E−29 |
| Tm4sf4 | 0.512552747 | NA | NA | 1.16E−28 | NA |
| Hal | NA | NA | 0.280113531 | NA | NA |
| Cmbl | −0.442706435 | −0.312422041 | NA | 2.71E−26 | 1.48E−28 |
| Baz1a | 0.259610685 | NA | NA | 1.78E−28 | NA |
| Ak3 | NA | 0.289040998 | NA | NA | 1.86E−28 |
| Nrp1 | 0.360791537 | 0.334097753 | NA | 2.25E−08 | 1.93E−28 |
| Gpt2 | 0.539114072 | NA | NA | 2.49E−28 | NA |
| Etf1 | 0.559444816 | NA | NA | 2.60E−28 | NA |
| Hook1 | NA | 0.327423989 | NA | NA | 3.21E−28 |
| Ndufb4 | −0.453693039 | −0.325145539 | NA | 2.93E−26 | 3.64E−28 |
| Lgr4 | NA | 0.268964381 | NA | NA | 9.73E−28 |
| Lrrc59 | 0.377173076 | 0.318083941 | NA | 1.88E−10 | 1.35E−27 |
| Dkc1 | 0.32575964 | NA | NA | 1.54E−27 | NA |
| Stard10 | −0.338316568 | NA | NA | 1.68E−27 | NA |
| Hpx | −0.303288229 | NA | NA | 1.79E−27 | NA |
| Etnk1 | 0.315992963 | 0.352068809 | NA | 1.00E−05 | 1.82E−27 |
| Agmat | −0.471832829 | NA | NA | 2.33E−27 | NA |
| Stard4 | NA | 0.351527506 | NA | NA | 2.40E−27 |
| Cyp39a1 | 0.351128984 | NA | NA | 2.44E−27 | NA |
| Malat1 | 0.920628097 | NA | NA | 2.62E−27 | NA |
| Dusp6 | 0.59164596 | NA | NA | 2.71E−27 | NA |
| Ywhag | 0.58241474 | NA | NA | 3.05E−27 | NA |
| Adipor2 | −0.473791512 | NA | NA | 3.14E−27 | NA |
| Arcn1 | NA | 0.327775216 | NA | NA | 3.29E−27 |
| Serp1 | NA | 0.312399212 | −0.267566027 | NA | 3.50E−27 |
| Ifitm3 | −0.425091425 | NA | −0.254348675 | 1.16E−25 | NA |
| Ces3b | −0.36637965 | −0.286685415 | −0.263292817 | 6.63E−22 | 1.58E−25 |
| Fcna | 0.377428055 | NA | NA | 5.71E−27 | NA |
| App | 0.361832458 | NA | NA | 6.12E−27 | NA |
| Cyp2c67 | −0.463488581 | NA | NA | 1.02E−26 | NA |
| Lrrfip1 | 0.445928709 | NA | NA | 1.19E−26 | NA |
| Dhrs3 | −0.467621234 | NA | NA | 1.25E−26 | NA |
| Rpl36a1 | −0.475541391 | NA | NA | 1.26E−26 | NA |
| Cml2 | −0.397916138 | 0.265441744 | NA | 4.33E−26 | 1.85E−26 |
| Ppp1r3b | −0.402657788 | NA | NA | 2.29E−26 | NA |
| Eif4g1 | 0.374860037 | 0.310782755 | NA | 3.98E−13 | 2.42E−26 |
| Ptpn12 | 0.33417878 | NA | NA | 5.69E−26 | NA |
| Pik3c2a | 0.324870962 | 0.25938652 | NA | 1.17E−09 | 5.98E−26 |
| Cyp4a12a | −0.456827492 | NA | 0.277051162 | 6.69E−26 | NA |
| Mug2 | 0.368295871 | 0.370406564 | NA | 9.47E−13 | 6.96E−26 |
| Lman1 | NA | 0.29860668 | NA | NA | 9.00E−26 |
| Hspe1 | −0.480734946 | −0.290582957 | 0.281318916 | 9.37E−26 | 1.53E−19 |
| Mrpl14 | −0.459040925 | NA | NA | 9.74E−26 | NA |
| Zfp683 | −0.421689112 | −0.421395009 | NA | 5.25E−09 | 1.05E−25 |
| Mak16 | 0.317126116 | NA | NA | 1.09E−25 | NA |
| Acsl4 | 0.298529302 | 0.314743242 | NA | 0.000351815 | 1.14E−25 |
| Copa | NA | 0.345094339 | NA | NA | 1.16E−25 |
| mt-Atp6 | −0.47818742 | −0.573743033 | 0.323426357 | 1.61E−07 | 1.31E−25 |
| Uqcrh | −0.429164519 | NA | NA | 1.41E−25 | NA |
| Slc38a3 | −0.40978981 | NA | NA | 1.45E−25 | NA |
| Minos1 | −0.455548025 | NA | NA | 2.00E−25 | NA |
| Ebpl | −0.450334658 | NA | NA | 2.09E−25 | NA |
| Atox1 | −0.446203219 | NA | NA | 2.48E−25 | NA |
| Tbc1d15 | 0.488646353 | NA | NA | 2.52E−25 | NA |
| Aox3 | −0.333319828 | 0.287845433 | NA | 2.16E−15 | 2.60E−25 |
| Map2k3 | 0.434654367 | NA | NA | 2.73E−25 | NA |
| Vkorc1 | −0.404176916 | −0.297366597 | NA | 3.54E−22 | 3.15E−25 |
| Cyp7a1 | −0.431201844 | NA | NA | 3.19E−25 | NA |
| Trip12 | NA | 0.328304235 | NA | NA | 3.31E−25 |
| Hpgd | −0.387752401 | NA | NA | 3.49E−25 | NA |
| Lmo4 | 0.320534245 | NA | NA | 3.97E−25 | NA |
| Cyb5b | −0.423345561 | NA | NA | 4.82E−25 | NA |
| Klf15 | −0.451831953 | NA | NA | 5.02E−25 | NA |
| Gnl2 | 0.390473036 | NA | NA | 5.63E−25 | NA |
| 3110043O21Rik | 0.269386631 | NA | NA | 5.64E−25 | NA |
| Asgr1 | −0.385655364 | NA | NA | 6.52E−25 | NA |
| Mtus1 | NA | 0.362060296 | NA | NA | 7.17E−25 |
| Cyp2j5 | −0.398305774 | NA | NA | 7.33E−25 | NA |
| Mup1 | −0.737727332 | −0.261837735 | −0.556836871 | 2.63E−21 | 1 |
| Prdx5 | −0.28208825 | −0.260937182 | NA | 2.68E−15 | 8.22E−25 |
| Chd1 | 0.378763061 | NA | NA | 8.51E−25 | NA |
| Rarres1 | −0.455632451 | NA | −0.291851226 | 8.53E−25 | NA |
| Sel1l | NA | 0.33595882 | NA | NA | 8.76E−25 |
| Arl5a | NA | 0.264805346 | NA | NA | 1.52E−24 |
| Mpeg1 | 0.493478112 | NA | NA | 1.80E−24 | NA |
| Wdr43 | 0.343830267 | NA | NA | 1.89E−24 | NA |
| Aff4 | 0.616309327 | NA | NA | 1.96E−24 | NA |
| Timm13 | −0.432961802 | NA | NA | 2.46E−24 | NA |
| 1500017E21Rik | −0.460997215 | NA | NA | 2.47E−24 | NA |
| Nop10 | −0.291908137 | −0.264928395 | NA | 3.59E−14 | 2.62E−24 |
| Serpina12 | −0.453399356 | NA | −0.324744036 | 1.67E−21 | NA |
| Fam107b | 0.463320341 | NA | NA | 2.90E−24 | NA |
| Cep85l | 0.277893187 | NA | NA | 3.24E−24 | NA |
| Srpr | NA | 0.305179041 | NA | NA | 4.78E−24 |
| Narf | NA | 0.262933124 | NA | NA | 6.15E−24 |
| 2410015M20Rik | −0.402123463 | NA | NA | 6.84E−24 | NA |
| Gtpbp4 | 0.482239467 | NA | NA | 6.84E−24 | NA |
| Prdx4 | −0.417588892 | −0.307442113 | −0.251975081 | 1.27E−20 | 6.96E−24 |
| Ccs | −0.426694207 | NA | NA | 7.67E−24 | NA |
| Rps15 | −0.455155041 | −0.356137747 | NA | 4.49E−19 | 7.79E−24 |
| Tns1 | 0.434939232 | NA | NA | 8.90E−24 | NA |
| Esd | −0.280760023 | −0.25379488 | NA | 1.14E−13 | 9.90E−24 |
| Ncl | 0.48450982 | NA | NA | 1.05E−23 | NA |
| Stom | 0.323229029 | NA | NA | 1.13E−23 | NA |
| Amacr | −0.413582585 | NA | NA | 1.14E−23 | NA |
| Sco2 | −0.260508515 | −0.292870629 | NA | 5.95E−07 | 1.32E−23 |
| Glo1 | −0.406574019 | NA | NA | 1.87E−23 | NA |
| Mcl1 | 0.570934356 | NA | NA | 2.05E−23 | NA |
| Mybbp1a | 0.431161557 | NA | NA | 2.11E−23 | NA |
| Nars | 0.437769953 | 0.353230364 | NA | 7.84E−17 | 2.87E−23 |
| Slc38a10 | NA | 0.285548034 | NA | NA | 2.98E−23 |
| Gm10800 | 0.562597493 | NA | NA | 3.19E−23 | NA |
| Stt3b | NA | 0.319405267 | NA | NA | 3.84E−23 |
| Srp72 | NA | 0.310685136 | NA | NA | 5.31E−23 |
| Prrc2c | 0.439271845 | NA | NA | 6.04E−23 | NA |
| Zyx | 0.298338034 | NA | NA | 6.24E−23 | NA |
| Sc4mol | −0.326170476 | −0.273479653 | NA | 7.58E−15 | 6.26E−23 |
| Mrp63 | −0.442201567 | NA | NA | 7.79E−23 | NA |
| Arg1 | 0.317948822 | NA | NA | 8.62E−23 | NA |
| Arhgef12 | 0.590764816 | 0.304679431 | NA | 8.83E−23 | 1.41E−20 |
| Acsm1 | −0.369245009 | NA | NA | 9.63E−23 | NA |
| Mat2a | 0.482570924 | NA | NA | 1.45E−22 | NA |
| Cars | 0.315814572 | NA | NA | 1.93E−22 | NA |
| Rps9 | −0.371793799 | −0.313951151 | NA | 1.65E−17 | 1.94E−22 |
| Ell2 | 0.417241283 | 0.288090516 | NA | 8.49E−12 | 2.55E−22 |
| Gm24245 | 0.546463773 | 0.42420314 | NA | 4.23E−18 | 3.25E−22 |
| Rpl13a | −0.381462137 | NA | NA | 3.58E−22 | NA |
| Bzw1 | 0.294810535 | 0.284147153 | NA | 1.35E−07 | 3.59E−22 |
| Grlf1 | NA | 0.295046691 | NA | NA | 3.99E−22 |
| Bbox1 | −0.408245616 | NA | NA | 4.17E−22 | NA |
| Map3k5 | 0.33471162 | NA | NA | 4.76E−22 | NA |
| Pecr | −0.398084036 | NA | NA | 4.93E−22 | NA |
| Ndufa3 | −0.41160475 | NA | NA | 5.38E−22 | NA |
| Tmem14c | −0.368514287 | NA | NA | 6.04E−22 | NA |
| Atp13a3 | 0.394480252 | 0.303730845 | NA | 2.58E−11 | 6.61E−22 |
| Pxk | 0.281854648 | NA | NA | 6.95E−22 | NA |
| Rps18 | −0.488540797 | NA | NA | 8.18E−22 | NA |
| Mrps24 | −0.390380395 | NA | NA | 8.75E−22 | NA |
| Mtss1 | NA | 0.287581972 | NA | NA | 1.08E−21 |
| Gjb1 | −0.31656535 | NA | NA | 1.13E−21 | NA |
| Edem1 | NA | 0.286881059 | NA | NA | 1.13E−21 |
| Spp2 | −0.382096801 | NA | NA | 1.28E−21 | NA |
| Kif5b | 0.50116686 | NA | NA | 1.57E−21 | NA |
| Acly | NA | 0.328803101 | NA | NA | 1.74E−21 |
| Rpl13 | −0.298816085 | −0.299693639 | NA | 1.55E−10 | 1.78E−21 |
| 1110001J03Rik | −0.432785224 | NA | NA | 2.04E−21 | NA |
| Dync1li1 | 0.262406206 | NA | NA | 2.23E−21 | NA |
| Ndufa2 | −0.396473631 | NA | NA | 2.46E−21 | NA |
| Txndc5 | NA | 0.290124872 | NA | NA | 3.56E−21 |
| Psme1 | −0.394006165 | NA | NA | 3.58E−21 | NA |
| Ppp1r12a | 0.356295305 | NA | NA | 3.60E−21 | NA |
| Bphl | −0.40747923 | NA | NA | 5.02E−21 | NA |
| Trf | −0.261883505 | NA | NA | 5.32E−21 | NA |
| Cyp2c68 | −0.398830868 | NA | NA | 7.05E−21 | NA |
| Trib1 | 0.331693479 | 0.287458438 | NA | 0.001450331 | 7.49E−21 |
| Akr1c19 | NA | −0.272667086 | NA | NA | 7.78E−21 |
| Cpne3 | NA | 0.258007443 | NA | NA | 8.12E−21 |
| Pnpla7 | NA | 0.29608325 | −0.301626901 | NA | 1.01E−20 |
| Dnajb11 | NA | 0.315357308 | NA | NA | 1.04E−20 |
| Phc2 | 0.31203626 | NA | NA | 1.14E−20 | NA |
| Atp6v1f | NA | −0.252893521 | NA | NA | 1.18E−20 |
| Tars | 0.492059498 | NA | NA | 1.28E−20 | NA |
| Ctnnb1 | NA | 0.295681078 | NA | NA | 1.58E−20 |
| Id3 | NA | −0.351088732 | NA | NA | 1.84E−20 |
| Tmem243 | −0.379053275 | −0.26555147 | NA | 2.52E−19 | 2.35E−20 |
| Eci1 | NA | −0.276383144 | NA | NA | 2.48E−20 |
| Lap3 | −0.364371999 | NA | NA | 2.65E−20 | NA |
| Cebpa | −0.39155301 | NA | NA | 3.89E−20 | NA |
| Tfpi2 | −0.366342462 | NA | NA | 4.58E−20 | NA |
| Il6ra | 0.304392668 | NA | NA | 5.08E−20 | NA |
| Cald1 | 0.373703655 | 0.258910981 | NA | 3.11E−13 | 5.69E−20 |
| Neat1 | 0.63740381 | NA | NA | 5.72E−20 | NA |
| Ptprf | NA | 0.307126385 | NA | NA | 6.50E−20 |
| Uqcrb | −0.408416744 | NA | NA | 6.59E−20 | NA |
| Ostc | NA | 0.265829876 | NA | NA | 7.22E−20 |
| Sec31a | NA | 0.263042758 | NA | NA | 7.68E−20 |
| Mbnl1 | 0.451258107 | 0.25020478 | NA | 9.50E−20 | 2.20E−15 |
| Osbpl9 | NA | 0.278773264 | NA | NA | 1.04E−19 |
| Alad | −0.420641438 | NA | NA | 1.16E−19 | NA |
| Gch1 | −0.269053605 | −0.282499843 | NA | 1.54E−09 | 1.62E−19 |
| Lrrfip2 | 0.373099277 | NA | NA | 1.85E−19 | NA |
| Ireb2 | NA | 0.265741209 | NA | NA | 1.87E−19 |
| Cisd1 | −0.372694857 | NA | NA | 2.37E−19 | NA |
| Mocs2 | −0.346681141 | NA | NA | 2.62E−19 | NA |
| Coa6 | −0.411409685 | NA | NA | 2.73E−19 | NA |
| Mrc1 | 0.340444916 | NA | NA | 2.90E−19 | NA |
| Copb2 | NA | 0.283928956 | NA | NA | 3.01E−19 |
| Stk17b | 0.291050054 | NA | NA | 3.56E−19 | NA |
| Ghr | −0.364775879 | NA | NA | 3.66E−19 | NA |
| Ptp4a1 | 0.262691158 | NA | NA | 3.82E−19 | NA |
| Osbpl1a | NA | 0.272788799 | NA | NA | 4.18E−19 |
| Atp5g3 | −0.300221819 | NA | NA | 4.43E−19 | NA |
| Cyp27a1 | −0.373510332 | −0.251578373 | NA | 3.99E−18 | 4.44E−19 |
| Akr1a1 | −0.326935362 | NA | NA | 4.63E−19 | NA |
| Atp5c1 | −0.347648538 | NA | NA | 5.04E−19 | NA |
| Nme1 | −0.432885306 | NA | −0.285705103 | 5.43E−19 | NA |
| Azin1 | NA | 0.270139699 | NA | NA | 6.16E−19 |
| Purb | 0.35248294 | NA | NA | 6.99E−19 | NA |
| Timm8b | −0.393581904 | NA | NA | 8.39E−19 | NA |
| Uhrf1bp1l | 0.419802385 | NA | NA | 8.66E−19 | NA |
| Sfpq | 0.393977901 | NA | NA | 9.08E−19 | NA |
| Gsk3b | 0.406458835 | NA | NA | 9.12E−19 | NA |
| Iqgap2 | NA | 0.254698841 | NA | NA | 9.72E−19 |
| Cideb | −0.368365389 | −0.27071201 | NA | 5.77E−18 | 1.02E−18 |
| Ndufc2 | −0.425284817 | −0.308039389 | NA | 1.31E−18 | 1.02E−18 |
| Palld | 0.341357023 | NA | NA | 1.34E−18 | NA |
| D10Jhu81e | −0.395730917 | NA | NA | 1.93E−18 | NA |
| Pxmp2 | −0.367129885 | NA | NA | 2.40E−18 | NA |
| 15-Sep | −0.409167133 | NA | NA | 2.53E−18 | NA |
| Smg1 | 0.334990596 | NA | NA | 2.85E−18 | NA |
| Aass | 0.477517138 | NA | NA | 3.07E−18 | NA |
| Hnrnpdl | 0.397994117 | NA | NA | 3.09E−18 | NA |
| Akap11 | 0.346362286 | NA | NA | 4.54E−18 | NA |
| Nedd4l | 0.281232817 | NA | NA | 4.90E−18 | NA |
| Jhdm1d | 0.304101624 | NA | NA | 4.92E−18 | NA |
| 1500011K16Rik | −0.329639846 | NA | NA | 5.16E−18 | NA |
| Rps3 | −0.349320751 | NA | NA | 5.25E−18 | NA |
| Slc22a30 | −0.374598404 | NA | −0.295400819 | 5.26E−18 | NA |
| Erp44 | NA | 0.279450299 | NA | NA | 5.51E−18 |
| Coro1c | 0.306679199 | NA | NA | 6.74E−18 | NA |
| Ptpn2 | 0.273418437 | NA | NA | 7.37E−18 | NA |
| Commd6 | −0.364529559 | NA | NA | 7.88E−18 | NA |
| Fitm1 | −0.324153911 | NA | NA | 7.90E−18 | NA |
| Dgat2 | −0.343266251 | NA | NA | 8.35E−18 | NA |
| Surf4 | NA | 0.258577589 | NA | NA | 9.74E−18 |
| Gm10801 | 0.265934225 | NA | NA | 1.30E−17 | NA |
| Cox6b1 | −0.362017449 | NA | NA | 1.47E−17 | NA |
| Adtrp | −0.366413277 | NA | NA | 1.53E−17 | NA |
| Ankrd11 | 0.415626852 | NA | NA | 1.70E−17 | NA |
| Ccbl1 | −0.36488302 | NA | NA | 2.18E−17 | NA |
| Ftl1 | −0.26029441 | −0.349777423 | −0.29880847 | 0.34648571 | 2.43E−17 |
| Prkaa2 | NA | 0.263640766 | NA | NA | 2.46E−17 |
| Arl6ip1 | −0.364945533 | NA | NA | 2.55E−17 | NA |
| Egr1 | 0.584558888 | NA | NA | 2.80E−17 | NA |
| Dnajb9 | NA | 0.25864192 | NA | NA | 2.89E−17 |
| Smdt1 | −0.367317155 | NA | NA | 3.21E−17 | NA |
| Ddc | −0.360752783 | NA | NA | 3.48E−17 | NA |
| Ndufc1 | −0.344432714 | NA | NA | 3.78E−17 | NA |
| Ndufb8 | −0.348076577 | NA | NA | 3.81E−17 | NA |
| Zbtb20 | 0.471532978 | 0.379090332 | NA | 1.83E−11 | 4.06E−17 |
| Xiap | NA | 0.26016306 | NA | NA | 4.75E−17 |
| Tjp1 | 0.286163863 | NA | NA | 4.89E−17 | NA |
| Anp32a | −0.316957742 | NA | NA | 5.23E−17 | NA |
| Ide | NA | 0.251691529 | NA | NA | 5.60E−17 |
| Ehhadh | NA | −0.250472633 | NA | NA | 7.29E−17 |
| Sptbn1 | 0.42268289 | NA | NA | 7.32E−17 | NA |
| Mbl2 | −0.346518253 | NA | NA | 7.59E−17 | NA |
| Kpnb1 | NA | 0.280415627 | NA | NA | 7.91E−17 |
| Mien1 | −0.370437762 | NA | NA | 8.08E−17 | NA |
| Gne | −0.425142761 | NA | NA | 9.04E−17 | NA |
| Bola3 | −0.368862428 | NA | NA | 1.08E−16 | NA |
| Akap13 | 0.495253767 | NA | NA | 1.14E−16 | NA |
| Hist1h1c | −0.300906532 | NA | NA | 1.17E−16 | NA |
| Nr2f6 | −0.375433952 | NA | NA | 1.37E−16 | NA |
| Ces1f | −0.312589958 | NA | NA | 1.45E−16 | NA |
| Ndufv3 | −0.397145193 | NA | NA | 1.45E−16 | NA |
| Cast | 0.400886525 | NA | NA | 1.50E−16 | NA |
| Golga4 | 0.352084678 | 0.271769698 | NA | 3.49E−05 | 1.56E−16 |
| Apom | −0.302410414 | NA | NA | 1.68E−16 | NA |
| Ebp | −0.355328266 | NA | NA | 1.79E−16 | NA |
| Gpam | NA | 0.250377871 | NA | NA | 1.86E−16 |
| Edf1 | −0.381679393 | NA | NA | 1.87E−16 | NA |
| Srebf1 | NA | 0.27619787 | NA | NA | 1.90E−16 |
| Ndufs8 | −0.361439192 | NA | NA | 1.93E−16 | NA |
| Eprs | 0.37008595 | 0.301270543 | NA | 1.04E−06 | 1.94E−16 |
| Ndufa1 | −0.364515751 | NA | NA | 1.96E−16 | NA |
| Litaf | 0.394366845 | NA | NA | 2.04E−16 | NA |
| H6pd | 0.343718187 | 0.261357904 | NA | 9.26E−07 | 2.22E−16 |
| Bptf | 0.324463857 | NA | NA | 2.66E−16 | NA |
| Ifrd1 | 0.490389826 | NA | NA | 2.92E−16 | NA |
| Plk3 | 0.416624571 | NA | NA | 3.12E−16 | NA |
| Nadk2 | NA | 0.257852312 | NA | NA | 3.23E−16 |
| Myl12a | 0.390780681 | NA | NA | 3.32E−16 | NA |
| Psmb1 | −0.358482353 | NA | NA | 3.95E−16 | NA |
| Baat | −0.308582001 | NA | NA | 4.31E−16 | NA |
| Cnpy2 | −0.372850093 | NA | NA | 4.94E−16 | NA |
| Bhmt2 | −0.311413339 | NA | NA | 5.24E−16 | NA |
| Gstm4 | −0.380861133 | NA | NA | 5.61E−16 | NA |
| Chac2 | −0.286511682 | NA | NA | 6.00E−16 | NA |
| Mettl20 | −0.331367185 | NA | NA | 6.50E−16 | NA |
| Bckdha | −0.349979609 | NA | NA | 6.82E−16 | NA |
| BC089597 | NA | 0.262237515 | NA | NA | 6.90E−16 |
| Sirt3 | −0.345997558 | NA | NA | 7.30E−16 | NA |
| Mdm2 | 0.328296258 | NA | NA | 8.15E−16 | NA |
| Acy3 | −0.313748988 | NA | NA | 8.20E−16 | NA |
| Hsp90ab1 | 0.322748941 | NA | NA | 8.34E−16 | NA |
| Mrpl12 | −0.308200176 | NA | NA | 9.04E−16 | NA |
| Fam120a | NA | 0.280043634 | NA | NA | 9.81E−16 |
| Atp5o | −0.331628292 | NA | NA | 9.93E−16 | NA |
| Fkbp4 | −0.353366286 | NA | NA | 1.01E−15 | NA |
| Cldn3 | −0.314469359 | NA | NA | 1.08E−15 | NA |
| Ipo5 | 0.268735631 | NA | NA | 1.12E−15 | NA |
| Fpgs | −0.34940945 | NA | NA | 1.15E−15 | NA |
| Sdsl | 0.259290764 | NA | NA | 1.41E−15 | NA |
| Tgm2 | 0.399330712 | NA | NA | 1.43E−15 | NA |
| Phlda1 | 0.549174347 | NA | NA | 1.46E−15 | NA |
| Macf1 | 0.404177263 | 0.261666188 | NA | 4.26E−11 | 1.47E−15 |
| Mrps18c | −0.366291099 | NA | NA | 1.60E−15 | NA |
| Sbno1 | 0.367100512 | 0.250529348 | NA | 2.44E−13 | 1.62E−15 |
| Nedd8 | −0.356380686 | NA | NA | 1.86E−15 | NA |
| Magt1 | NA | 0.263295188 | NA | NA | 1.95E−15 |
| Sdhb | −0.289642823 | NA | NA | 2.09E−15 | NA |
| Ethe1 | −0.333417645 | NA | NA | 2.45E−15 | NA |
| Mdh1 | −0.307657821 | NA | NA | 3.99E−15 | NA |
| Nrip1 | 0.272854584 | NA | NA | 4.11E−15 | NA |
| Lcp1 | 0.484011001 | NA | NA | 4.22E−15 | NA |
| Srsf11 | 0.401209055 | NA | NA | 4.47E−15 | NA |
| Shb | 0.315236194 | NA | NA | 4.68E−15 | NA |
| Bsg | −0.31515012 | NA | NA | 5.31E−15 | NA |
| Hibch | −0.327130099 | NA | NA | 6.26E−15 | NA |
| Mki67ip | 0.318372964 | NA | NA | 6.27E−15 | NA |
| Otc | −0.29903416 | NA | NA | 6.99E−15 | NA |
| Smap2 | 0.348911433 | NA | NA | 9.52E−15 | NA |
| Nr0b2 | −0.293010319 | NA | NA | 1.07E−14 | NA |
| Pank1 | −0.352650118 | NA | NA | 1.09E−14 | NA |
| Ak2 | −0.345102135 | NA | NA | 1.13E−14 | NA |
| Rlim | 0.31023282 | NA | NA | 1.42E−14 | NA |
| Erbb2ip | 0.333163506 | 0.279650574 | NA | 2.35E−07 | 1.69E−14 |
| Huwe1 | 0.387931325 | NA | NA | 1.75E−14 | NA |
| Hmgcs1 | −0.349112403 | NA | NA | 1.98E−14 | NA |
| Rps21 | −0.364450868 | NA | NA | 2.02E−14 | NA |
| Grhpr | −0.353058662 | NA | NA | 2.13E−14 | NA |
| Tm7sf2 | −0.316381125 | NA | NA | 2.28E−14 | NA |
| Txn1 | −0.309123787 | NA | NA | 2.50E−14 | NA |
| Sh3bgrl | NA | 0.25128227 | NA | NA | 2.71E−14 |
| 0610011F06Rik | −0.303688511 | NA | NA | 2.80E−14 | NA |
| Psmb6 | −0.319377533 | NA | NA | 2.87E−14 | NA |
| Gm2788 | −0.283138796 | NA | NA | 2.89E−14 | NA |
| AW112010 | −0.298104828 | NA | NA | 3.17E−14 | NA |
| Dhdh | −0.334814135 | NA | NA | 4.04E−14 | NA |
| Sf1 | 0.30230624 | NA | NA | 4.12E−14 | NA |
| Tbl1xr1 | 0.307509115 | NA | NA | 4.15E−14 | NA |
| Coq9 | −0.347936449 | NA | NA | 4.20E−14 | NA |
| Il4ra | 0.25739098 | NA | NA | 4.44E−14 | NA |
| Tceb2 | −0.32073626 | NA | NA | 4.50E−14 | NA |
| Rps10 | −0.33690905 | NA | NA | 4.59E−14 | NA |
| Psme4 | NA | 0.262584214 | NA | NA | 4.62E−14 |
| Gm4951 | −0.322087312 | NA | NA | 5.37E−14 | NA |
| Pak2 | 0.281467155 | NA | NA | 5.57E−14 | NA |
| Metap2 | 0.368604828 | NA | NA | 6.59E−14 | NA |
| 2010107E04Rik | −0.318247806 | NA | NA | 7.21E−14 | NA |
| Prox1 | NA | 0.266329524 | NA | NA | 7.66E−14 |
| Ddx6 | 0.444275069 | NA | NA | 7.70E−14 | NA |
| Pacsin2 | 0.370825053 | NA | NA | 7.89E−14 | NA |
| Hdac11 | −0.33684651 | NA | NA | 8.63E−14 | NA |
| Ubl5 | −0.303313218 | NA | NA | 1.12E−13 | NA |
| Mettl7a1 | −0.322098553 | NA | NA | 1.21E−13 | NA |
| Larp4b | 0.34214672 | NA | NA | 1.22E−13 | NA |
| Uqcc2 | −0.326858302 | NA | NA | 1.27E−13 | NA |
| 1810022K09Rik | −0.300081452 | NA | NA | 1.29E−13 | NA |
| Selk | −0.338314398 | NA | −0.289300641 | 1.79E−12 | NA |
| Dhx9 | 0.350364065 | NA | NA | 1.41E−13 | NA |
| Psmb3 | −0.330302519 | NA | NA | 1.47E−13 | NA |
| Rexo2 | −0.3263129 | NA | NA | 1.48E−13 | NA |
| Hipk3 | 0.339385214 | NA | NA | 1.58E−13 | NA |
| Cyp4f14 | −0.295933211 | NA | NA | 1.60E−13 | NA |
| Wdr26 | 0.432510772 | NA | NA | 1.94E−13 | NA |
| Sec61b | −0.290284119 | NA | NA | 2.20E−13 | NA |
| Paqr9 | −0.332341635 | NA | NA | 2.23E−13 | NA |
| Tsc22d1 | −0.299728636 | NA | NA | 2.38E−13 | NA |
| Glyctk | 0.297701904 | NA | NA | 2.68E−13 | NA |
| Mapk6 | 0.256831906 | NA | NA | 3.22E−13 | NA |
| Atp1b1 | 0.370203015 | NA | NA | 3.27E−13 | NA |
| Aqp9 | −0.310863805 | NA | NA | 3.38E−13 | NA |
| Suco | 0.250962583 | NA | NA | 3.39E−13 | NA |
| Dync1h1 | 0.379607116 | NA | NA | 4.50E−13 | NA |
| Ctsb | 0.521310448 | NA | NA | 4.83E−13 | NA |
| Tpr | 0.440921219 | NA | NA | 5.22E−13 | NA |
| Supt6 | 0.291294768 | NA | NA | 5.48E−13 | NA |
| Lsmd1 | −0.313140806 | NA | NA | 5.95E−13 | NA |
| Kif21a | 0.404170578 | NA | NA | 6.46E−13 | NA |
| Sgk1 | 0.284303028 | NA | NA | 7.18E−13 | NA |
| Proz | NA | 0.263204472 | NA | NA | 7.77E−13 |
| Romo1 | −0.335854841 | NA | NA | 7.99E−13 | NA |
| Cfhr1 | −0.325332852 | NA | NA | 8.01E−13 | NA |
| Deptor | NA | 0.260618372 | NA | NA | 8.58E−13 |
| Atp2b1 | 0.332096305 | NA | NA | 9.01E−13 | NA |
| Rnf13 | NA | 0.255296715 | NA | NA | 9.16E−13 |
| Dhrs4 | −0.332074421 | NA | NA | 9.59E−13 | NA |
| Lasp1 | NA | 0.253021439 | NA | NA | 9.93E−13 |
| Tmem147 | 0.283785217 | NA | NA | 1.00E−12 | NA |
| Fus | 0.339324351 | NA | NA | 1.03E−12 | NA |
| Yap1 | 0.328109852 | NA | NA | 1.07E−12 | NA |
| Acads | −0.32421998 | NA | NA | 1.10E−12 | NA |
| Fam25c | −0.297673676 | NA | NA | 1.12E−12 | NA |
| Ndufb3 | −0.327185415 | NA | NA | 1.16E−12 | NA |
| Agpat2 | −0.298159013 | NA | NA | 1.22E−12 | NA |
| Bst2 | −0.315565973 | NA | NA | 1.52E−12 | NA |
| Caprin1 | NA | 0.262825966 | NA | NA | 1.56E−12 |
| Syncrip | 0.27476476 | NA | NA | 1.65E−12 | NA |
| Rnaseh2c | −0.325683719 | NA | NA | 1.67E−12 | NA |
| Cldn2 | −0.251076838 | NA | NA | 1.86E−12 | NA |
| Klf3 | 0.267357487 | NA | NA | 1.89E−12 | NA |
| Tnks2 | 0.317626402 | NA | NA | 1.91E−12 | NA |
| Derl2 | −0.331734152 | NA | NA | 2.28E−12 | NA |
| Ndufv2 | −0.327639406 | NA | NA | 2.48E−12 | NA |
| Eif4b | 0.36816236 | 0.255987641 | NA | 3.78E−07 | 2.53E−12 |
| Cyp4f15 | −0.299840345 | NA | NA | 2.53E−12 | NA |
| Brd2 | 0.571515166 | 0.266388486 | NA | 1.73E−11 | 2.65E−12 |
| Copz1 | NA | 0.257722711 | NA | NA | 2.65E−12 |
| Ddx27 | 0.252155421 | NA | NA | 3.46E−12 | NA |
| Mcee | −0.31991829 | NA | NA | 3.52E−12 | NA |
| Dnajc19 | −0.316006732 | NA | NA | 3.54E−12 | NA |
| Aldh4a1 | −0.29070878 | NA | NA | 3.75E−12 | NA |
| Fdps | −0.266347718 | NA | NA | 4.58E−12 | NA |
| Reep3 | 0.409014614 | NA | NA | 5.12E−12 | NA |
| Car5a | −0.308769058 | NA | NA | 5.42E−12 | NA |
| Tmem37 | −0.282207793 | NA | NA | 6.77E−12 | NA |
| Tspo | −0.315729205 | NA | NA | 6.95E−12 | NA |
| Tspan31 | −0.295881364 | NA | NA | 7.18E−12 | NA |
| Atp11c | NA | 0.260536506 | NA | NA | 7.22E−12 |
| Atf6 | 0.377819844 | NA | NA | 7.66E−12 | NA |
| Rpl14 | −0.277637754 | NA | NA | 7.87E−12 | NA |
| Med13 | 0.294918978 | NA | NA | 7.95E−12 | NA |
| Sdr9c7 | −0.259988635 | NA | NA | 8.11E−12 | NA |
| Rps8 | NA | NA | −0.274601282 | NA | NA |
| Kpna1 | 0.32304803 | NA | NA | 8.67E−12 | NA |
| Btaf1 | 0.275463861 | NA | NA | 8.92E−12 | NA |
| Ddah1 | −0.312000429 | NA | NA | 9.85E−12 | NA |
| Ndufs4 | −0.313160933 | NA | NA | 1.01E−11 | NA |
| Fam214a | −0.265769677 | NA | NA | 1.02E−11 | NA |
| Rbm25 | 0.421459678 | NA | NA | 1.03E−11 | NA |
| Ndufb10 | −0.300358086 | NA | NA | 1.04E−11 | NA |
| Slirp | −0.308519262 | NA | NA | 1.04E−11 | NA |
| Scaf11 | 0.359845436 | NA | NA | 1.30E−11 | NA |
| Anapc13 | −0.364181956 | NA | NA | 1.31E−11 | NA |
| Arpc1b | 0.259676842 | NA | NA | 1.36E−11 | NA |
| Pnkd | −0.292772175 | NA | NA | 1.41E−11 | NA |
| Abcd3 | −0.271805105 | NA | NA | 1.44E−11 | NA |
| Hmgcr | 0.416711628 | NA | NA | 1.61E−11 | NA |
| Mxd1 | 0.297767874 | NA | NA | 1.65E−11 | NA |
| Bet1 | −0.296563627 | NA | NA | 1.73E−11 | NA |
| Cela1 | −0.272296838 | NA | NA | 1.78E−11 | NA |
| Tmem160 | −0.339044834 | NA | NA | 2.24E−11 | NA |
| Snx10 | 0.285642594 | NA | NA | 2.35E−11 | NA |
| Ubb | −0.275030382 | −0.259339434 | −0.264432797 | 6.19E−10 | 2.58E−11 |
| Lgals9 | −0.269998113 | NA | NA | 2.87E−11 | NA |
| Aadat | −0.318627951 | NA | NA | 3.04E−11 | NA |
| Mrpl33 | −0.265447876 | NA | NA | 3.06E−11 | NA |
| Tmem258 | −0.27689533 | NA | NA | 3.18E−11 | NA |
| Cyp2d40 | −0.321717407 | NA | NA | 3.46E−11 | NA |
| Birc6 | 0.344367807 | NA | NA | 3.56E−11 | NA |
| Naprt1 | −0.338143058 | NA | NA | 4.15E−11 | NA |
| Agxt | −0.258383387 | NA | NA | 4.28E−11 | NA |
| Kdelr2 | 0.397729248 | NA | NA | 4.46E−11 | NA |
| Stbd1 | −0.273216024 | NA | NA | 5.39E−11 | NA |
| Phf20l1 | 0.308488005 | NA | NA | 5.49E−11 | NA |
| Tmprss6 | −0.280234101 | NA | NA | 5.62E−11 | NA |
| Gstk1 | −0.282802747 | NA | NA | 5.77E−11 | NA |
| Gadd45gip1 | −0.304090556 | NA | NA | 6.25E−11 | NA |
| Ubr4 | 0.330144527 | NA | NA | 7.63E−11 | NA |
| Sigmar1 | −0.305468332 | NA | NA | 7.64E−11 | NA |
| Ythdc1 | 0.264130576 | NA | NA | 8.40E−11 | NA |
| Psma6 | −0.30353658 | NA | NA | 8.48E−11 | NA |
| Acaa1a | −0.301848759 | NA | NA | 9.21E−11 | NA |
| Cyp2c55 | −0.290435372 | NA | NA | 9.36E−11 | NA |
| Proc | −0.270719362 | NA | NA | 9.66E−11 | NA |
| Mrpl20 | −0.309348635 | NA | NA | 1.03E−10 | NA |
| Myeov2 | −0.305189789 | NA | NA | 1.05E−10 | NA |
| Dnajc5 | 0.326483514 | NA | NA | 1.16E−10 | NA |
| Kdm5a | 0.351390096 | NA | NA | 1.27E−10 | NA |
| Commd3 | −0.265556375 | NA | NA | 1.46E−10 | NA |
| Mrpl42 | −0.284507519 | NA | NA | 1.47E−10 | NA |
| Bdhl | −0.293611347 | NA | NA | 1.50E−10 | NA |
| Mrpl15 | −0.291228504 | NA | NA | 1.56E−10 | NA |
| Abcb11 | −0.261778471 | NA | NA | 1.57E−10 | NA |
| Ddost | NA | 0.250356033 | NA | NA | 1.89E−10 |
| Anxa5 | 0.365372347 | NA | NA | 2.08E−10 | NA |
| Gsr | 0.412829579 | NA | NA | 2.10E−10 | NA |
| Atf4 | 0.341134734 | NA | NA | 2.33E−10 | NA |
| Iah1 | −0.292985785 | NA | NA | 2.41E−10 | NA |
| Pdzd8 | 0.286270568 | NA | NA | 2.46E−10 | NA |
| Fbxo21 | −0.263087536 | NA | NA | 2.85E−10 | NA |
| Ftsj3 | 0.255227431 | NA | NA | 2.86E−10 | NA |
| C6 | NA | NA | −0.26698766 | NA | NA |
| Acot13 | −0.288638646 | NA | NA | 3.05E−10 | NA |
| Acat3 | −0.311510623 | NA | NA | 3.16E−10 | NA |
| Magi3 | 0.269961841 | NA | NA | 3.24E−10 | NA |
| Cybb | 0.377799564 | NA | NA | 3.48E−10 | NA |
| Tmem242 | −0.272538433 | NA | NA | 3.57E−10 | NA |
| Mrpl41 | −0.267412137 | NA | NA | 3.73E−10 | NA |
| Sema4g | −0.250459516 | NA | NA | 3.89E−10 | NA |
| Cap1 | 0.325055169 | NA | NA | 4.46E−10 | NA |
| Csrp3 | −0.293996249 | NA | NA | 4.47E−10 | NA |
| Hpn | −0.268601326 | NA | NA | 4.81E−10 | NA |
| Slc25a51 | 0.263985158 | NA | NA | 6.14E−10 | NA |
| Spcs1 | −0.26287037 | NA | NA | 6.30E−10 | NA |
| Apof | −0.252173807 | NA | NA | 6.73E−10 | NA |
| Cep85 | 0.28389234 | NA | NA | 6.87E−10 | NA |
| Aes | −0.264214993 | NA | NA | 7.53E−10 | NA |
| Pet100 | −0.28003555 | NA | NA | 7.60E−10 | NA |
| Gcat | −0.258728349 | NA | NA | 7.71E−10 | NA |
| Rps24 | −0.262473665 | NA | NA | 8.21E−10 | NA |
| Dcaf11 | v0.281659388 | NA | NA | 9.55E−10 | NA |
| Pmpcb | v0.263615722 | NA | NA | 1.03E−09 | NA |
| Lipc | −0.254841484 | NA | NA | 1.09E−09 | NA |
| Ptpn11 | 0.277804552 | NA | NA | 1.15E−09 | NA |
| Ccdc50 | 0.285830087 | NA | NA | 1.18E−09 | NA |
| Ivns1abp | 0.356610399 | NA | NA | 1.20E−09 | NA |
| Nqo2 | −0.267802053 | NA | NA | 1.22E−09 | NA |
| Abcg2 | −0.280923445 | NA | NA | 1.38E−09 | NA |
| Dera | −0.267441985 | NA | NA | 1.43E−09 | NA |
| Fasn | NA | 0.291406736 | NA | NA | 1.44E−09 |
| Rabac1 | −0.265987205 | NA | NA | 1.45E−09 | NA |
| Gm20594 | 0.295686612 | −0.360000649 | −0.356558629 | 1.60E−05 | 2.58E−08 |
| Clic4 | 0.299795775 | NA | NA | 1.64E−09 | NA |
| Ugt3a2 | −0.258613351 | NA | NA | 1.89E−09 | NA |
| Mvd | −0.270409867 | NA | NA | 1.91E−09 | NA |
| Ptpmt1 | −0.255981462 | NA | NA | 2.02E−09 | NA |
| 0610007P14Rik | −0.257015045 | NA | NA | 2.31E−09 | NA |
| Swi5 | −0.262086804 | NA | NA | 2.33E−09 | NA |
| Tpi1 | −0.26157943 | NA | NA | 2.35E−09 | NA |
| Plcxd2 | −0.275192522 | NA | NA | 2.48E−09 | NA |
| Tmem150a | −0.253507791 | NA | NA | 2.61E−09 | NA |
| Rnf169 | 0.333974467 | NA | NA | 2.63E−09 | NA |
| Deb1 | −0.267029156 | NA | NA | 2.74E−09 | NA |
| Hsbp1 | −0.286726926 | NA | NA | 2.99E−09 | NA |
| Slc26a1 | −0.28165635 | NA | NA | 3.01E−09 | NA |
| Rsl1d1 | 0.346056141 | NA | NA | 3.82E−09 | NA |
| Map3k2 | 0.251791147 | NA | NA | 3.83E−09 | NA |
| Abcf1 | 0.303190272 | NA | NA | 4.09E−09 | NA |
| Psma1 | −0.256671259 | NA | NA | 4.64E−09 | NA |
| Fam195a | −0.264890901 | NA | NA | 4.66E−09 | NA |
| Mrpl11 | −0.268482417 | NA | NA | 4.92E−09 | NA |
| Selo | −0.257421903 | NA | NA | 5.24E−09 | NA |
| Plbd2 | 0.264873115 | NA | NA | 5.33E−09 | NA |
| Rap1b | 0.340766029 | NA | NA | 6.89E−09 | NA |
| Ppip5k2 | 0.320939415 | NA | NA | 7.41E−09 | NA |
| Pcm1 | 0.265397644 | NA | NA | 8.07E−09 | NA |
| Acsm5 | −0.261394035 | NA | NA | 8.53E−09 | NA |
| Rangap1 | 0.296226768 | NA | NA | 1.08E−08 | NA |
| Rraga | 0.262613483 | NA | NA | 1.10E−08 | NA |
| 1110058L19Rik | −0.258088587 | NA | NA | 1.12E−08 | NA |
| Dsp | 0.265533344 | NA | NA | 1.15E−08 | NA |
| Ccnl1 | 0.256623048 | NA | NA | 1.44E−08 | NA |
| Chmp4b | 0.300153098 | NA | NA | 1.52E−08 | NA |
| Ubr5 | 0.305718363 | NA | NA | 1.72E−08 | NA |
| Net1 | 0.34785505 | NA | NA | 1.84E−08 | NA |
| H2-D1 | 0.286522764 | NA | NA | 2.31E−08 | NA |
| Srd5a1 | −0.277347272 | NA | NA | 2.35E−08 | NA |
| Papss2 | −0.254475327 | NA | NA | 2.73E−08 | NA |
| Dnaja2 | 0.372342331 | NA | NA | 2.79E−08 | NA |
| Srrm2 | 0.328159189 | NA | NA | 3.12E−08 | NA |
| Herc1 | 0.256188826 | NA | NA | 3.26E−08 | NA |
| Gtf2a2 | 0.279207862 | NA | NA | 3.58E−08 | NA |
| Pkp4 | 0.323033636 | NA | NA | 4.02E−08 | NA |
| Trip11 | 0.347886673 | NA | NA | 4.15E−08 | NA |
| Slc16a2 | −0.251969763 | NA | NA | 4.57E−08 | NA |
| Ctsc | 0.361021766 | 0.300373343 | NA | 4.80E−08 | 9.04E−06 |
| Megf9 | 0.276890033 | NA | NA | 6.26E−08 | NA |
| Mrpl28 | −0.269896048 | NA | NA | 6.87E−08 | NA |
| Rpl34 | −0.297598539 | NA | NA | 8.13E−08 | NA |
| Mrpl34 | −0.283750306 | NA | NA | 8.42E−08 | NA |
| Mgea5 | 0.32630901 | NA | NA | 9.07E−08 | NA |
| C1d | −0.255261652 | NA | NA | 9.45E−08 | NA |
| Gcgr | −0.257636913 | NA | NA | 1.04E−07 | NA |
| Sptan1 | 0.312663889 | NA | NA | 1.10E−07 | NA |
| Zfp259 | 0.255636639 | NA | NA | 1.10E−07 | NA |
| Xrn2 | 0.298471923 | NA | NA | 1.28E−07 | NA |
| Hipk1 | 0.291600227 | NA | NA | 1.40E−07 | NA |
| Alcam | 0.323788815 | NA | NA | 1.46E−07 | NA |
| Gnas | 0.258853087 | NA | NA | 1.47E−07 | NA |
| Mapkapk2 | 0.31382654 | NA | NA | 1.63E−07 | NA |
| Coasy | −0.253428655 | NA | NA | 1.78E−07 | NA |
| Gars | 0.257638792 | NA | NA | 2.09E−07 | NA |
| Diap1 | 0.325941798 | NA | NA | 2.19E−07 | NA |
| Rab6a | 0.32343226 | NA | NA | 2.23E−07 | NA |
| Pvrl2 | −0.261260991 | NA | NA | 2.66E−07 | NA |
| 2810428115Rik | −0.263117928 | NA | NA | 2.80E−07 | NA |
| Nucks1 | 0.355785584 | NA | NA | 2.82E−07 | NA |
| Hnrnpu | 0.339415607 | NA | NA | 3.35E−07 | NA |
| Chuk | 0.372437977 | NA | NA | 5.21E−07 | NA |
| Serbp1 | 0.322652522 | NA | NA | 5.43E−07 | NA |
| Ctnnd1 | 0.251848095 | NA | NA | 5.48E−07 | NA |
| Nploc4 | 0.291804954 | NA | NA | 5.69E−07 | NA |
| Zfp106 | 0.336920255 | NA | NA | 6.34E−07 | NA |
| Arhgdia | 0.256326455 | NA | NA | 7.14E−07 | NA |
| BC005537 | 0.354337911 | NA | NA | 1.05E−06 | NA |
| Akr1e1 | −0.258207619 | NA | NA | 1.09E−06 | NA |
| Eific | 0.301979757 | NA | NA | 1.10E−06 | NA |
| Tmem86b | −0.258236533 | NA | NA | 1.18E−06 | NA |
| Mrps12 | −0.250441318 | NA | NA | 1.26E−06 | NA |
| Cdh1 | 0.269696738 | NA | NA | 1.61E−06 | NA |
| Ddx3y | 0.339517654 | NA | NA | 1.83E−06 | NA |
| Ndufa5 | −0.251988986 | NA | NA | 1.96E−06 | NA |
| Ppm1g | 0.252225215 | NA | NA | 2.04E−06 | NA |
| Elovl5 | 0.335236661 | NA | NA | 2.54E−06 | NA |
| Impad1 | 0.32267465 | NA | NA | 3.04E−06 | NA |
| Tab2 | 0.348412001 | NA | NA | 3.51E−06 | NA |
| Cyp3a13 | 0.284411943 | NA | NA | 4.93E−06 | NA |
| Pgls | −0.274714156 | NA | NA | 5.62E−06 | NA |
| Actr3 | 0.275997927 | NA | NA | 5.66E−06 | NA |
| St3gal4 | 0.275033534 | NA | NA | 5.81E−06 | NA |
| 4932438A13Rik | 0.287850722 | NA | NA | 7.97E−06 | NA |
| Abce1 | 0.279955892 | NA | NA | 8.43E−06 | NA |
| Pde3b | −0.258970093 | NA | NA | 8.55E−06 | NA |
| Smoc1 | 0.26157412 | NA | NA | 8.57E−06 | NA |
| 4931406C07Rik | −0.254363633 | NA | NA | 9.62E−06 | NA |
| Rmnd5a | 0.318903459 | NA | NA | 1.09E−05 | NA |
| Ash11 | 0.265693821 | NA | NA | 1.23E−05 | NA |
| Sf3b1 | 0.28884767 | NA | NA | 1.32E−05 | NA |
| Spag9 | 0.318820155 | NA | NA | 1.33E−05 | NA |
| Nek7 | 0.291098417 | NA | NA | 1.63E−05 | NA |
| Eif4a1 | 0.251906299 | NA | NA | 2.25E−05 | NA |
| Usp7 | 0.287320099 | NA | NA | 2.90E−05 | NA |
| Tbca | −0.27261031 | NA | NA | 3.43E−05 | NA |
| Pi4k2a | 0.353561144 | NA | NA | 3.46E−05 | NA |
| Prpf19 | 0.25876049 | NA | NA | 3.47E−05 | NA |
| Usp34 | 0.285942387 | NA | NA | 5.16E−05 | NA |
| Slc16a10 | −0.254826191 | NA | NA | 9.62E−05 | NA |
| Eea1 | 0.261201113 | NA | NA | 0.000132332 | NA |
| Sars | 0.276853119 | NA | NA | 0.00019395 | NA |
| Herc4 | 0.267474882 | NA | NA | 0.000255647 | NA |
| Gadd45a | 0.282867411 | NA | NA | 0.000337547 | NA |
| Bc1af1 | 0.26848767 | NA | NA | 0.000425718 | NA |
| Gm5424 | 0.269800095 | NA | NA | 0.000520866 | NA |
| Slc35d1 | 0.309435259 | NA | NA | 0.000579989 | NA |
| Cu11 | 0.259206487 | NA | NA | 0.000597201 | NA |
| Cu13 | 0.278802183 | NA | NA | 0.000803381 | NA |
| Slc30a1 | 0.250570123 | NA | NA | 0.000818871 | NA |
| Eifia | 0.314713983 | NA | NA | 0.00096785 | NA |
| Tln1 | 0.321033046 | NA | NA | 0.001767724 | NA |
| Zfand5 | 0.28815165 | NA | NA | 0.00189648 | NA |
| Glul | NA | NA | 0.379494728 | NA | NA |
| Eif4ebp1 | 0.261682735 | NA | NA | 0.003063164 | NA |
| Hectd1 | 0.30802968 | NA | NA | 0.003395612 | NA |
| Gldc | 0.280156601 | NA | NA | 0.00370491 | NA |
| Rassf3 | 0.291236708 | NA | NA | 0.004723315 | NA |
| Nudt4 | 0.25418892 | NA | NA | 0.007016738 | NA |
| Rock1 | 0.2607635 | NA | NA | 0.009308994 | NA |
| Rbm39 | 0.265732671 | NA | NA | 0.012545192 | NA |
| Abcc2 | 0.289681267 | NA | NA | 0.018266512 | NA |
| Naa50 | 0.265659058 | NA | NA | 0.019114384 | NA |
| Pa2g4 | 0.289349263 | NA | NA | 0.02381992 | NA |
| Riok3 | 0.291562038 | NA | NA | 0.055677881 | NA |
| Myo1b | 0.262427327 | NA | NA | 0.075213075 | NA |
| Eifib | 0.250328103 | NA | NA | 0.08550598 | NA |
| Ldlr | 0.282025946 | NA | NA | 0.146582345 | NA |
| Psap | 0.257800256 | NA | NA | 0.360973931 | NA |
| Gda | 0.266276129 | NA | NA | 0.627476336 | NA |
| Gnai2 | 0.281667665 | NA | NA | 1 | NA |

| ID | A48p | avg_logFC | p_val_adj |
|---|---|---|---|
| Mup17 | 0 | −2.354358557 | 0 |
| Mup6 | 0 | −2.032084932 | 0 |
| Mup9 | 0 | −2.004163996 | 0 |
| Mup19 | 0 | −2.396345291 | 0 |
| Mup11 | 0 | −3.213953203 | 0 |
| Mup18 | 0 | −1.727178347 | 0 |
| Selenbp2 | 0 | −1.190221952 | 0 |
| Mup7 | 0 | −1.115587848 | 0 |
| Mup16 | 0 | −1.602085539 | 0 |
| mt-Co1 | 0 | −1.43342755 | 0 |
| Mup21 | 0 | −1.008171912 | 0 |
| mt-Nd4 | 0 | −1.957424825 | 0 |
| mt-Nd2 | 0 | −1.241926048 | 0 |
| mt-Cytb | 0 | −1.571232911 | 0 |
| mt-Nd5 | 7.18E−267 | −1.383591054 | 0 |
| Serpina1e | 0 | −0.662045945 | 0 |
| mt-Nd1 | 5.29E−267 | −1.419683574 | 0 |
| Mup3 | 7.73E−237 | −0.806219448 | 0 |
| Apoe | 0 | −0.421271729 | 0 |
| Ces1c | 0 | 0.466837177 | 0 |
| Hmgcs2 | 0 | 0.629289122 | 0 |
| Angptl3 | 0 | 0.7089371 | 0 |
| Ang | 0 | 0.7089371 | 0 |
| Chchd10 | 0 | 0.720691838 | 0 |
| Chchd1 | 0 | 0.720691838 | 0 |
| Mat1a | 0 | 1.000433202 | 0 |
| Cyp4a10 | 0 | 1.135370857 | 0 |
| Cyp4a14 | 0 | 1.704093543 | 0 |
| Hbb-bs | NA | 2.862044849 | 0 |
| Hmox1 | NA | 1.347585217 | 0 |
| Gm26924 | NA | 1.419027502 | 0 |
| Fga | NA | 0.828543313 | 0 |
| n-R5-8s1 | NA | 1.252818829 | 0 |
| Fgg | NA | 0.680530925 | 0 |
| Fgb | NA | 0.744973762 | 0 |
| Grip2 | NA | 0.877868293 | 0 |
| Apoa2 | NA | −0.545518454 | 0 |
| Apoh | 8.57E−300 | 0.411790107 | 8.57E−300 |
| Ttpa | 4.46E−295 | 0.601240096 | 4.46E−295 |
| Lars2 | NA | 1.711400221 | 1.38E−293 |
| Gm13775 | 1.37E−292 | −1.07808822 | 1.37E−292 |
| Cdo1 | 3.00E−292 | 0.517045053 | 3.00E−292 |
| Gm15564 | NA | 1.820580657 | 6.31E−292 |
| S100a9 | NA | 1.342849617 | 5.31E−281 |
| Tnfrsf12a | NA | 1.117506296 | 5.70E−279 |
| Gm24601 | NA | 0.984391691 | 2.17E−275 |
| Apoc2 | 3.07E−267 | 0.782126085 | 3.07E−267 |
| Retsat | 9.77E−266 | 0.681307283 | 9.77E−266 |
| Ttr | 5.79E−264 | −0.530625643 | 5.79E−264 |
| Sephs2 | NA | 0.889992859 | 2.83E−263 |
| S100a8 | NA | 1.127879209 | 1.92E−258 |
| Nudt7 | 2.67E−255 | −1.564266936 | 2.67E−255 |
| mt-Rnr2 | 1.98E−232 | −0.778341998 | 5.04E−251 |
| Ppl | NA | 0.879840886 | 9.97E−249 |
| Serpina1a | NA | −0.675201963 | 4.32E−247 |
| Acaa1b | NA | −0.85084133 | 4.51E−247 |
| Sepp1 | 3.03E−246 | 0.448586822 | 3.03E−246 |
| Srxn1 | NA | 1.878352012 | 2.22E−243 |
| Apoc1 | 7.90E−242 | −0.871282735 | 7.90E−242 |
| Mup5 | 1.94E−240 | −1.370621307 | 1.94E−240 |
| Fgl1 | NA | 0.864933885 | 1.60E−238 |
| Gpx1 | NA | −0.768556331 | 6.77E−238 |
| Mt2 | NA | 2.06273693 | 1.90E−237 |
| Ankrd55 | 1.45E−68 | −1.8735659 | 4.38E−235 |
| Mup15 | 1.22E−233 | −1.316464146 | 1.22E−233 |
| Mfsd2a | 5.34E−233 | 0.666142747 | 5.34E−233 |
| Mt1 | 1.20E−56 | 2.387707773 | 3.51E−232 |
| Ndrg2 | 2.62E−223 | 0.41219937 | 2.62E−223 |
| Ahsg | 1.68E−221 | 0.292489576 | 1.68E−221 |
| Hrg | 3.81E−221 | 0.518895355 | 3.81E−221 |
| mt-Co2 | 1.79E−173 | −1.396543228 | 7.79E−219 |
| Sult1a1 | 9.07E−216 | 0.560453494 | 9.07E−216 |
| Agt | 1.09E−213 | 0.520454244 | 1.09E−213 |
| Cyb5 | NA | −0.71018958 | 8.36E−212 |
| Fth1 | 5.81E−204 | 0.378367166 | 5.81E−204 |
| Snord118 | 2.66E−203 | 0.617425052 | 2.66E−203 |
| Fabp1 | NA | −1.016174848 | 1.10E−198 |
| Slco1a4 | 1.65E−197 | 0.552641329 | 1.65E−197 |
| mt-Rnr1 | 1.71E−195 | −0.485399855 | 1.71E−195 |
| Saa1 | 3.34E−194 | −1.078220383 | 3.34E−194 |
| Hilpda | NA | 0.778625914 | 3.64E−193 |
| Gclc | NA | 1.902837282 | 1.67E−192 |
| Cp | 6.52E−192 | 0.436243259 | 6.52E−192 |
| Igfbp4 | 1.54E−189 | 0.43528879 | 1.54E−189 |
| Akr1c6 | 8.38E−143 | −1.586259995 | 2.54E−187 |
| Rbp4 | 1.88E−186 | 0.301440617 | 1.88E−186 |
| Mup12 | 1.14E−185 | −1.269973728 | 1.14E−185 |
| Serpina1d | NA | −0.494007182 | 1.74E−184 |
| Hspa5 | 2.27E−84 | 0.610549064 | 9.07E−184 |
| Mgst1 | NA | −1.060154854 | 2.13E−181 |
| Rdh7 | 6.63E−181 | −0.418625957 | 6.63E−181 |
| Apoc3 | 8.39E−179 | −0.768781074 | 8.39E−179 |
| Herpud1 | 9.68E−179 | 0.513571228 | 9.68E−179 |
| Aldob | 9.03E−178 | 0.365460648 | 9.03E−178 |
| Abcd2 | 1.40E−177 | 0.285090597 | 1.40E−177 |
| Gc | 2.82E−177 | 0.259816642 | 2.82E−177 |
| Abcb4 | 3.66E−176 | 0.494736917 | 3.66E−176 |
| mt-Co3 | 4.34E−176 | −1.167027737 | 4.34E−176 |
| Ugt2b5 | NA | −0.603351248 | 9.60E−175 |
| Slc27a2 | 1.69E−174 | 0.385256962 | 1.69E−174 |
| Ccl6 | NA | 0.78914235 | 1.24E−171 |
| St3gal5 | 1.06E−170 | 0.607639775 | 1.06E−170 |
| Hbb-bt | NA | 1.430134393 | 2.56E−170 |
| Reep6 | 2.74E−169 | 0.514272451 | 2.74E−169 |
| Slc16a6 | NA | 0.911357108 | 4.06E−168 |
| Ttc36 | 1.00E−167 | −0.660645659 | 1.00E−167 |
| Adh1 | NA | −1.255028206 | 1.90E−167 |
| Gsta3 | NA | −0.806290466 | 2.44E−166 |
| Cvp2e1 | NA | −2.318570161 | 4.79E−166 |
| Plin2 | NA | 1.461295758 | 8.58E−163 |
| Il1r1 | NA | 1.244999819 | 1.47E−162 |
| F2 | 3.99E−160 | 0.32133198 | 3.99E−160 |
| Decr1 | 4.55E−160 | 0.466364339 | 4.55E−160 |
| Actb | 8.47E−52 | 1.059835055 | 6.06E−159 |
| Slc25a47 | 1.45E−158 | 0.502661475 | 1.45E−158 |
| Srgn | NA | 0.934067453 | 5.07E−158 |
| Pabpc1 | 1.11E−157 | 0.494914201 | 1.11E−157 |
| Cyp2d9 | NA | −0.640698605 | 3.96E−157 |
| Hyou1 | NA | 0.846484822 | 1.62E−156 |
| Lcat | 8.53E−155 | 0.436082968 | 8.53E−155 |
| Grn | 1.07E−154 | 0.540768685 | 1.07E−154 |
| Hsd3b5 | 1.41E−154 | −1.069520224 | 1.41E−154 |
| Ube2r2 | 1.35E−153 | 0.46659877 | 1.35E−153 |
| Hamp | NA | −1.02112368 | 2.59E−153 |
| Apoa5 | 8.39E−153 | 0.323188383 | 8.39E−153 |
| Cdkn1a | NA | 1.2152557 | 2.99E−152 |
| Serpina1c | NA | −0.402626732 | 7.17E−151 |
| Gstp1 | 1.88E−150 | −0.855831436 | 1.88E−150 |
| Tubb6 | NA | 0.576687679 | 6.13E−150 |
| Apoc4 | NA | −1.002265767 | 9.54E−150 |
| Nid1 | NA | 0.342599117 | 1.04E−149 |
| Calr | NA | 0.555145472 | 1.67E−148 |
| Klf6 | NA | 0.732407935 | 1.50E−147 |
| Cers6 | NA | 0.532818648 | 8.16E−146 |
| Hmgcl | 1.74E−145 | 0.473794522 | 1.74E−145 |
| Aldh9a1 | 1.28E−144 | 0.415934269 | 1.28E−144 |
| Txnrd1 | NA | 1.443578942 | 1.43E−144 |
| Il1b | NA | 0.742893545 | 1.02E−143 |
| Hspa8 | 1.80E−143 | 0.420379148 | 1.80E−143 |
| Psat1 | NA | 0.301248562 | 8.43E−143 |
| Gm23935 | 3.70E−142 | 1 | 3.70E−142 |
| Tmbim6 | 4.11E−142 | 0.338652766 | 4.11E−142 |
| Rgn | NA | −1.573093006 | 9.97E−141 |
| Amy1 | 1.36E−140 | 0.432738347 | 1.36E−140 |
| Uqcrq | NA | −0.88665513 | 1.50E−140 |
| Scd1 | 5.06E−126 | 0.726687286 | 2.08E−139 |
| Itih4 | NA | 0.525074867 | 4.26E−137 |
| Gyk | 8.15E−137 | 0.402444184 | 8.15E−137 |
| Hp | NA | 0.431328341 | 8.95E−137 |
| Chka | NA | 1.375010255 | 2.35E−136 |
| Pdia3 | NA | 0.593769188 | 7.36E−136 |
| Decr2 | 6.71E−135 | 0.418851877 | 6.71E−135 |
| Cyp3a25 | 1.19E−134 | −0.386094858 | 1.19E−134 |
| Serping1 | 1.56E−134 | 0.41280952 | 1.56E−134 |
| Wbp1l | 4.08E−134 | 0.553507326 | 4.08E−134 |
| Hsp90b1 | NA | 0.534208217 | 6.37E−134 |
| Gnmt | NA | −0.46036106 | 1.33E−133 |
| Atp5k | 1.43E−133 | −0.47725773 | 1.43E−133 |
| 2010003K11Rik | NA | 0.588354735 | 2.61E−131 |
| Serpina3m | 1.04E−130 | 0.967441963 | 1.04E−130 |
| Slco1b2 | 1.55E−55 | −1.087378176 | 3.68E−129 |
| Lipa | 3.72E−129 | 0.41833763 | 3.72E−129 |
| Mup20 | 3.88E−128 | −0.476207303 | 3.88E−128 |
| Rgs16 | 5.95E−128 | 0.322559157 | 5.95E−128 |
| Bhmt | NA | −0.520184894 | 1.20E−127 |
| Vtn | 2.90E−126 | 0.333811366 | 2.90E−126 |
| Plin5 | 1.22E−125 | 0.352087102 | 1.22E−125 |
| C3 | 1.44E−100 | 0.449521065 | 1.23E−125 |
| Agpat6 | 2.13E−125 | 0.396260224 | 2.13E−125 |
| Acat1 | 2.19E−125 | 0.368871999 | 2.19E−125 |
| Ets2 | NA | 0.695623802 | 2.30E−125 |
| Mup4 | 9.25E−125 | −1.087038527 | 9.25E−125 |
| Atp5h | 1.41E−124 | −0.6398091 | 1.41E−124 |
| Car3 | 4.54E−111 | −1.036093133 | 1.61E−124 |
| Acbd5 | 5.03E−124 | −0.3952179 | 5.03E−124 |
| Nr1i3 | 2.82E−123 | 0.425883248 | 2.82E−123 |
| Crat | 3.69E−123 | 0.255626614 | 3.69E−123 |
| Slc27a5 | 4.04E−123 | 0.336994904 | 4.04E−123 |
| Oaf | 1.03E−122 | −0.7328674 | 1.03E−122 |
| Cyp3a11 | 2.07E−107 | −0.423130745 | 1.05E−122 |
| Krt18 | NA | 1.257881489 | 3.97E−121 |
| Elovl3 | 5.92E−91 | −0.675967012 | 6.30E−121 |
| Cox5a | 6.48E−121 | 0.40236048 | 6.48E−121 |
| Ahcy | NA | −0.562968597 | 6.43E−120 |
| Sdhc | 1.27E−118 | 0.394392518 | 1.27E−118 |
| Gstt3 | 3.52E−118 | 0.350739865 | 3.52E−118 |
| Asl | 3.45E−117 | 0.558638879 | 3.45E−117 |
| Atp5b | 3.82E−117 | 0.291727476 | 3.82E−117 |
| C9 | 4.51E−117 | −0.427055258 | 4.51E−117 |
| Cyp2b10 | NA | −1.164490931 | 9.40E−117 |
| 1100001G20Rik | NA | −0.851607217 | 1.14E−116 |
| Slc25a20 | 4.01E−116 | 0.380017899 | 4.01E−116 |
| Hba-a1 | NA | 0.734957385 | 2.58E−115 |
| Ildr2 | 2.61E−115 | 0.267607713 | 2.61E−115 |
| Ppara | 4.20E−115 | 0.35885744 | 4.20E−115 |
| Dusp5 | NA | 0.296374788 | 1.01E−114 |
| Mup8 | 1.23E−114 | −0.79575704 | 1.23E−114 |
| P4hb | 1.25E−84 | 0.405223744 | 2.27E−113 |
| 2810007J24Rik | NA | −0.914301122 | 6.76E−113 |
| Rpl38 | 9.16E−113 | 0.254329206 | 9.16E−113 |
| Tgoln1 | 1.14E−112 | 0.38833408 | 1.14E−112 |
| Cox4i1 | NA | −0.515989169 | 4.62E−112 |
| Brap | 5.49E−112 | 0.349229859 | 5.49E−112 |
| Cox8a | 5.50E−112 | 0.44171223 | 5.50E−112 |
| Bfar | 4.46E−110 | 0.290332897 | 4.46E−110 |
| D17Wsu92e | 5.84E−110 | 0.371951808 | 5.84E−110 |
| Glud1 | NA | 0.451048591 | 6.55E−110 |
| Slc40a1 | 1.18E−109 | 0.466969696 | 1.18E−109 |
| Ephx2 | 3.91E−109 | −0.369303553 | 3.91E−109 |
| Bnip3 | 1.26E−108 | 0.403206563 | 1.26E−108 |
| Rnase4 | NA | −1.049196753 | 1.89E−107 |
| Dhcr24 | 3.03E−107 | 0.423756767 | 3.03E−107 |
| Myh9 | NA | 1.160233135 | 3.10E−107 |
| Acad11 | 7.32E−107 | 0.357135026 | 7.32E−107 |
| Fn1 | 3.08E−64 | 0.699634526 | 1.87E−106 |
| Mia3 | 3.36E−106 | 0.364623797 | 3.36E−106 |
| Gm6484 | 3.87E−106 | 0.304268201 | 3.87E−106 |
| Zfp91 | 6.43E−106 | 0.370686172 | 6.43E−106 |
| Gm2a | 1.13E−105 | 0.383109253 | 1.13E−105 |
| Sec14l2 | 4.44E−105 | −0.789737513 | 4.44E−105 |
| Cyp2a12 | 7.61E−104 | 0.374332361 | 7.61E−104 |
| Krt8 | NA | 1.195874806 | 1.04E−103 |
| Lpin2 | 1.43E−103 | 0.686687426 | 1.43E−103 |
| Clec4f | NA | 1.108180782 | 2.17E−103 |
| Cidec | NA | 0.299384206 | 1.71E−102 |
| B2m | NA | −0.417973886 | 1.86E−102 |
| Slc4a4 | 2.71E−102 | 0.420259653 | 2.71E−102 |
| Sh3glb1 | 3.84E−102 | 0.326006747 | 3.84E−102 |
| Tmed5 | 5.01E−102 | 0.401633325 | 5.01E−102 |
| Serpinf1 | 5.71E−102 | 0.319550979 | 5.71E−102 |
| 0610005C13Rik | 2.41E−101 | −0.425135941 | 2.41E−101 |
| Rpl4 | 2.66E−101 | 0.283935593 | 2.66E−101 |
| Dbi | NA | −0.716365271 | 3.56E−101 |
| Apoa4 | 8.65E−87 | 1.367998503 | 8.56E−101 |
| Acot12 | 1.49E−100 | 0.307840041 | 1.49E−100 |
| Acot1 | 1.49E−100 | 0.307840041 | 1.49E−100 |
| Scp2 | 3.33E−100 | −0.519124916 | 3.33E−100 |
| Uroc1 | 3.97E−100 | 0.416226092 | 3.97E−100 |
| Bcl2l1 | NA | 0.775118224 | 1.02E−99 |
| Mmp8 | NA | 0.359005863 | 1.02E−99 |
| Cyp2c29 | NA | −1.149629979 | 1.97E−99 |
| Snord13 | 2.42E−99 | 0.261078586 | 2.42E−99 |
| Acadl | 4.33E−99 | 0.316216015 | 4.33E−99 |
| Cpt1a | 2.13E−98 | 0.372935212 | 2.13E−98 |
| Akr1d1 | 2.99E−98 | 0.361037667 | 2.99E−98 |
| Cyp7b1 | 3.09E−98 | −0.65802273 | 3.09E−98 |
| Mgll | 5.21E−98 | 0.333899751 | 5.21E−98 |
| Sult1d1 | 1.12E−97 | 0.45987026 | 1.12E−97 |
| Slc25a10 | 1.85E−97 | 0.343272504 | 1.85E−97 |
| Vnn3 | 1.94E−97 | 0.276458974 | 1.94E−97 |
| Pigr | 2.38E−97 | 0.386842112 | 2.38E−97 |
| Pzp | NA | 0.41600896 | 3.34E−97 |
| Serpina1b | NA | −0.301770579 | 4.66E−97 |
| Tmem205 | NA | −0.774742732 | 1.05E−96 |
| Mup14 | 2.64E−96 | −0.760766854 | 2.64E−96 |
| Ces1b | 3.47E−96 | 0.372958858 | 3.47E−96 |
| C8g | NA | −0.654510262 | 4.13E−96 |
| Cd14 | NA | 0.362089625 | 2.79E−95 |
| Mtdh | 2.84E−95 | 0.30466184 | 2.84E−95 |
| Hdlbp | NA | 0.565147241 | 5.10E−95 |
| Tmbim4 | 5.14E−95 | 0.32269634 | 5.14E−95 |
| Saa2 | 6.06E−64 | 0.964488054 | 8.96E−95 |
| Abcc3 | 9.62E−95 | 0.363799323 | 9.62E−95 |
| Serpina10 | NA | 0.565046404 | 8.21E−94 |
| Cyp2c44 | 1.11E−93 | 0.385935584 | 1.11E−93 |
| Rcan1 | NA | 0.707283787 | 1.24E−93 |
| Arrdc3 | NA | 0.526871989 | 2.74E−93 |
| Hsd3b7 | 1.78E−61 | −1.0059958 | 3.79E−93 |
| Rell1 | NA | 0.591181865 | 1.02E−92 |
| Prdx1 | 1.10E−92 | −0.529229113 | 1.10E−92 |
| Fah | 1.16E−92 | 0.302494707 | 1.16E−92 |
| Chpt1 | 1.36E−92 | 0.35662056 | 1.36E−92 |
| Mthfd2 | NA | 0.262448025 | 1.46E−92 |
| Gfra1 | 2.65E−92 | 0.386382804 | 2.65E−92 |
| Retnlg | NA | 0.375148336 | 4.49E−92 |
| Pbld1 | 4.53E−92 | −0.442542986 | 4.53E−92 |
| Creld2 | NA | 0.608652695 | 1.18E−91 |
| Aplp2 | 1.48E−91 | 0.33938781 | 1.48E−91 |
| Lifr | 2.60E−91 | −0.626180925 | 2.60E−91 |
| Ces1d | 7.83E−69 | −0.428906017 | 8.07E−91 |
| D10Wsu102e | NA | 0.58780186 | 2.09E−90 |
| Camk2n1 | 4.24E−90 | 0.250109512 | 4.24E−90 |
| Rps27l | NA | −0.865927484 | 5.43E−90 |
| Bche | 7.85E−90 | 0.313419985 | 7.85E−90 |
| Itih3 | NA | 0.546240791 | 1.11E−89 |
| Plscr1 | NA | 0.444489505 | 2.65E−89 |
| Gm24270 | NA | 0.26796863 | 3.13E−89 |
| Tmem56 | 6.36E−89 | 1 | 6.36E−89 |
| Rdx | 1.02E−88 | 0.373557306 | 1.02E−88 |
| Abca6 | 1.30E−88 | 0.33284944 | 1.30E−88 |
| Gpld1 | 1.61E−88 | 0.328106789 | 1.61E−88 |
| Lrrc58 | NA | 1.001096085 | 3.66E−88 |
| Hba-a2 | NA | 0.553546734 | 5.15E−88 |
| Fxyd5 | NA | 0.274279595 | 7.76E−88 |
| Cyp8b1 | 3.14E−45 | −0.889229825 | 8.76E−88 |
| Cox6a1 | 1.78E−87 | −0.48722865 | 1.78E−87 |
| Ctage5 | 2.41E−87 | 0.328678207 | 2.41E−87 |
| Pex11a | 3.08E−87 | 0.350827626 | 3.08E−87 |
| Ass1 | 5.20E−87 | −0.314400604 | 5.20E−87 |
| Mttp | 1.44E−86 | 0.311504107 | 1.44E−86 |
| Eci2 | 1.53E−86 | 0.311004843 | 1.53E−86 |
| Prodh2 | 1.88E−86 | 0.345294809 | 1.88E−86 |
| Gadd45g | NA | −0.614019025 | 7.38E−86 |
| Psen2 | NA | −0.947373772 | 1.77E−85 |
| Creg1 | 2.77E−85 | 0.276773054 | 2.77E−85 |
| BC025446 | 4.26E−85 | 0.29422883 | 4.26E−85 |
| Serpina3n | 9.93E−85 | 0.848313239 | 9.93E−85 |
| Slc10a1 | 1.24E−84 | −0.325757408 | 1.24E−84 |
| Met | 1.73E−84 | 0.288332662 | 1.73E−84 |
| Mettl7b | 1.73E−84 | −0.452656656 | 1.73E−84 |
| Pck1 | 1.26E−61 | −0.652190949 | 1.90E−84 |
| Oat | NA | −1.224853019 | 6.25E−84 |
| Pcbp2 | 1.30E−83 | 0.310347013 | 1.30E−83 |
| Atf5 | 1.74E−83 | 0.329004772 | 1.74E−83 |
| Tyrobp | NA | 0.466551501 | 1.85E−83 |
| C230081A13Rik | NA | 0.426976229 | 3.42E−83 |
| Hnf4a | 3.56E−83 | 0.879108718 | 3.56E−83 |
| Atxn7l3b | 6.16E−83 | 0.306239775 | 6.16E−83 |
| G0s2 | 8.79E−83 | 0.346452213 | 8.79E−83 |
| F10 | 9.43E−83 | 0.279095541 | 9.43E−83 |
| Pla2g7 | NA | 0.282344956 | 9.47E−83 |
| Rplp0 | 1.83E−82 | −0.430901519 | 1.83E−82 |
| Gcdh | 4.27E−82 | −0.300320098 | 4.27E−82 |
| Creb3l3 | 5.52E−82 | 0.275700825 | 5.52E−82 |
| Ssr3 | NA | 0.521769281 | 5.71E−82 |
| Eef1a1 | NA | 0.439251983 | 2.20E−81 |
| Slc39a14 | NA | 1.012749203 | 2.25E−81 |
| Man2a1 | 2.32E−81 | 0.497115325 | 2.32E−81 |
| Cox6c | NA | −0.844280539 | 3.24E−81 |
| Il1r2 | NA | 0.323447978 | 5.62E−81 |
| Actn4 | 7.73E−81 | 0.312388865 | 7.73E−81 |
| Ptprc | NA | 0.416505491 | 1.46E−80 |
| Eif5 | 1.55E−80 | 0.576301866 | 1.55E−80 |
| Slc25a13 | 1.65E−80 | 0.285473174 | 1.65E−80 |
| Hint1 | NA | −0.673880575 | 1.71E−80 |
| Echs1 | 3.04E−80 | −0.331606352 | 3.04E−80 |
| Aco2 | 5.35E−80 | 0.285855791 | 5.35E−80 |
| Pnpla8 | 6.50E−80 | 0.295631611 | 6.50E−80 |
| C1qb | NA | 0.681679654 | 1.02E−79 |
| Hsd17b10 | 1.43E−79 | −0.377206484 | 1.43E−79 |
| Egfr | 5.82E−71 | 0.504957782 | 1.72E−79 |
| Cyp2a5 | NA | −1.498910166 | 3.74E−79 |
| Asgr2 | 6.28E−79 | 0.306445529 | 6.28E−79 |
| Tmsb4x | NA | 0.780616209 | 7.99E−79 |
| Hsd17b13 | 8.89E−79 | 0.680828119 | 8.89E−79 |
| Dennd5b | 1.08E−78 | 0.293458876 | 1.08E−78 |
| Ctsh | 1.35E−78 | 0.32445036 | 1.35E−78 |
| Igf1 | 1.83E−78 | 0.295791825 | 1.83E−78 |
| Apc | NA | 0.606730845 | 2.16E−78 |
| Apcs | NA | 0.75840106 | 2.16E−78 |
| Ctss | NA | 0.764167083 | 2.26E−78 |
| Cyp2d26 | 2.76E−78 | 0.259136874 | 2.76E−78 |
| Tcea3 | 2.91E−78 | −0.355007815 | 2.91E−78 |
| Klf9 | 3.25E−78 | 0.293415875 | 3.25E−78 |
| Orm1 | 1.56E−69 | 0.365144434 | 4.87E−78 |
| Clec4d | NA | 0.26359638 | 5.98E−78 |
| Mlycd | 7.66E−78 | 0.313046697 | 7.66E−78 |
| Maob | 2.74E−77 | 0.32037326 | 2.74E−77 |
| Kif1b | 2.75E−77 | 0.371764707 | 2.75E−77 |
| Insig2 | 5.68E−77 | 0.669991504 | 5.68E−77 |
| Ces3a | NA | −0.52533635 | 5.80E−77 |
| G6pc | NA | −0.960058274 | 1.00E−76 |
| Cth | NA | −0.510899065 | 1.34E−76 |
| Pgrmc1 | NA | 0.350152674 | 1.59E−76 |
| Ugt2b1 | NA | −0.776816931 | 2.28E−76 |
| Mup13 | 6.20E−45 | −0.77574306 | 2.45E−76 |
| Gabarap | 3.49E−76 | −0.442546824 | 3.49E−76 |
| Arhgap5 | 4.88E−76 | 0.295235575 | 4.88E−76 |
| Cyp1a2 | 2.09E−66 | −0.993935836 | 1.08E−75 |
| Hgd | NA | −0.758572265 | 1.96E−75 |
| Adck3 | NA | −0.87611538 | 2.27E−75 |
| Abhd14b | 2.40E−75 | −0.39366782 | 2.40E−75 |
| Serinc3 | 4.67E−75 | 0.35928911 | 4.67E−75 |
| Gltscr2 | 5.18E−75 | 0.255310647 | 5.18E−75 |
| Vcam1 | NA | 0.457401491 | 6.00E−75 |
| Urah | NA | −0.764109132 | 1.55E−74 |
| Sord | 1.84E−74 | −0.383628337 | 1.84E−74 |
| Agpat9 | NA | 0.327040595 | 2.14E−74 |
| Etnppl | 3.56E−74 | 0.496712405 | 3.56E−74 |
| Por | 1.06E−73 | 0.335963246 | 1.06E−73 |
| Rsp14 | 1.07E−73 | −0.402093042 | 1.07E−73 |
| Lamp1 | 1.16E−73 | 0.268283663 | 1.16E−73 |
| Plek | NA | 0.308857869 | 1.40E−73 |
| Ctsa | 2.08E−73 | 0.252009224 | 2.08E−73 |
| Mrap | NA | 0.884099926 | 2.65E−73 |
| Dnajc3 | NA | 0.45638751 | 2.66E−73 |
| Adamts1 | NA | 0.282556845 | 8.88E−73 |
| Hadh | 9.69E−73 | 0.288772268 | 9.69E−73 |
| C8b | 1.12E−72 | −0.360171247 | 1.12E−72 |
| Csad | NA | −0.559786018 | 1.92E−72 |
| Nr1d2 | NA | 0.430636791 | 2.60E−72 |
| Sod1 | NA | −0.439319795 | 3.30E−72 |
| Ech1 | 1.35E−56 | −0.429132615 | 5.62E−72 |
| Ddb1 | 1.19E−71 | 0.277809186 | 1.19E−71 |
| Kng1 | NA | 0.265777676 | 1.32E−71 |
| Shmt1 | 8.69E−71 | −0.393237106 | 8.69E−71 |
| Acsl1 | 1.12E−70 | −0.370316988 | 1.12E−70 |
| C4b | 3.16E−70 | 0.283273876 | 3.16E−70 |
| Gcnt2 | NA | 0.537509676 | 4.50E−70 |
| Csf1r | NA | 0.704458586 | 4.70E−70 |
| Ggnbp2 | 9.71E−70 | 0.272604502 | 9.71E−70 |
| Nfe2l1 | 1.23E−69 | 0.27034473 | 1.23E−69 |
| Tomm70a | 1.99E−69 | 0.268786029 | 1.99E−69 |
| Steap4 | NA | 0.475973731 | 6.59E−69 |
| Atp2a2 | 7.58E−69 | 0.494962728 | 7.58E−69 |
| Cd1d1 | 7.99E−69 | 0.291156805 | 7.99E−69 |
| Comt | 1.01E−68 | −0.368377448 | 1.01E−68 |
| Sec62 | 1.84E−68 | 0.251575572 | 1.84E−68 |
| Cxcr2 | NA | 0.40229963 | 2.86E−68 |
| Slc7a2 | 3.47E−68 | 0.352564931 | 3.47E−68 |
| C130074G19Rik | 4.06E−68 | −0.356338948 | 4.06E−68 |
| Apob | NA | 0.580156235 | 4.36E−68 |
| Furin | 8.30E−68 | 0.405633752 | 8.30E−68 |
| Cxcl1 | 9.38E−68 | 0.449766784 | 9.38E−68 |
| Cxcl12 | 9.38E−68 | −0.305838679 | 9.38E−68 |
| Khk | 1.03E−67 | −0.491453217 | 1.03E−67 |
| Ei24 | 1.18E−67 | 0.257327011 | 1.18E−67 |
| Cd163 | NA | 0.440845529 | 1.51E−67 |
| Trp53inp1 | NA | 1.008414088 | 1.52E−67 |
| Eif4g2 | NA | 0.559692663 | 1.62E−67 |
| Gys2 | 1.83E−67 | 0.274401611 | 1.83E−67 |
| Dmgdh | 2.36E−67 | 0.265846058 | 2.36E−67 |
| Tob1 | 3.20E−67 | −0.338705886 | 3.20E−67 |
| Ubc | NA | −0.661839716 | 4.04E−67 |
| Cbr1 | 7.07E−67 | 0.284717454 | 7.07E−67 |
| Pon1 | NA | −0.685751275 | 1.66E−66 |
| Suds3 | 1.93E−66 | 0.25388963 | 1.93E−66 |
| 1700017B05Rik | NA | 0.330656885 | 2.40E−66 |
| Ugp2 | 6.86E−66 | 0.297335445 | 6.86E−66 |
| Iigp1 | NA | 0.768753667 | 7.59E−66 |
| Nop58 | NA | 0.663449653 | 2.13E−65 |
| Padi3 | NA | 0.295682522 | 2.63E−65 |
| Pipox | 2.97E−65 | 0.272543042 | 2.97E−65 |
| Ndufa4 | NA | −0.683126695 | 3.70E−65 |
| Gsta1 | NA | 0.407606856 | 7.64E−65 |
| F9 | 8.09E−65 | 0.256805166 | 8.09E−65 |
| B4galt5 | NA | 0.476306558 | 1.12E−64 |
| F12 | 2.98E−64 | 0.256737037 | 2.98E−64 |
| Fcgrt | 4.12E−64 | −0.269314211 | 4.12E−64 |
| Fmo5 | 4.71E−64 | 0.286219883 | 4.71E−64 |
| Rnf144a | NA | 0.313046134 | 7.90E−64 |
| Ugt2b36 | NA | −0.583431487 | 1.36E−63 |
| Sardh | 1.48E−63 | 0.269183147 | 1.48E−63 |
| Iqgap1 | NA | 0.45544473 | 1.82E−63 |
| Creb3l2 | NA | 0.427180051 | 1.91E−63 |
| Slc22a1 | NA | −0.797941816 | 2.27E−63 |
| Lyz2 | NA | 0.732183369 | 2.49E−63 |
| Bach1 | NA | 0.832333098 | 2.51E−63 |
| Lpgat1 | 3.53E−63 | 0.595219291 | 3.53E−63 |
| Atp1a1 | 3.57E−63 | 0.419079339 | 3.57E−63 |
| Fam134b | NA | 0.893562894 | 5.02E−63 |
| Hsd17b4 | 6.45E−63 | 0.253232967 | 6.45E−63 |
| Gabarapl1 | 9.11E−63 | 0.260716513 | 9.11E−63 |
| Cd5l | NA | 0.693485826 | 1.08E−62 |
| Elovl2 | NA | 0.45224665 | 1.22E−62 |
| Lrg1 | NA | 0.490792829 | 1.37E−62 |
| Hgfac | 1.72E−62 | 0.252189991 | 1.72E−62 |
| Atp5d | 2.36E−62 | 0.250062798 | 2.36E−62 |
| Efhd2 | NA | 0.583831769 | 3.26E−62 |
| Pcbd1 | NA | −0.687761883 | 6.29E−62 |
| mmu-mir-6240 | NA | 1.101237189 | 9.82E−62 |
| Hagh | NA | −0.674009711 | 1.62E−61 |
| Hsd3b3 | 3.00E−20 | −0.779501123 | 1.98E−61 |
| Tmem106b | 2.12E−61 | 0.280204492 | 2.12E−61 |
| Ctsd | 4.43E−61 | 0.257862212 | 4.43E−61 |
| Cd44 | NA | 0.303745221 | 5.24E−61 |
| Rdh16 | 5.97E−61 | 0.269182372 | 5.97E−61 |
| Cyp2c70 | 6.16E−61 | −0.551134023 | 6.16E−61 |
| Thrsp | 6.83E−61 | −0.724961807 | 6.83E−61 |
| Hadha | 7.14E−61 | 0.250937029 | 7.14E−61 |
| Sdhd | 8.20E−61 | −0.335261252 | 8.20E−61 |
| Mbl1 | NA | −0.713006005 | 1.05E−60 |
| Ddt | NA | −0.522221147 | 1.33E−60 |
| Eif1a | NA | 0.795281298 | 1.80E−60 |
| Alb | NA | 0.420298986 | 1.86E−60 |
| Pdia4 | NA | 0.488166898 | 1.94E−60 |
| F5 | 3.21E−60 | 0.300566587 | 3.21E−60 |
| Laptm5 | NA | 0.338180771 | 3.31E−60 |
| Slc2a2 | 5.54E−60 | −0.312348392 | 5.54E−60 |
| Sfxn1 | 7.27E−60 | 0.304885886 | 7.27E−60 |
| Epas1 | NA | 0.775763153 | 7.57E−60 |
| Alas1 | NA | −0.489837477 | 8.49E−60 |
| Cd97 | NA | 0.345774315 | 1.02E−59 |
| Hacl1 | NA | −0.706897734 | 1.20E−59 |
| Oaz1 | 1.75E−59 | −0.418528822 | 1.75E−59 |
| Hpd | NA | −0.518180386 | 2.47E−59 |
| Cd74 | NA | 0.453937562 | 3.15E−59 |
| Aldh3a2 | 1.43E−09 | −0.834229334 | 3.38E−59 |
| Gclm | 3.66E−59 | 0.269864161 | 3.66E−59 |
| Serpina3c | 8.19E−59 | −0.472511045 | 8.19E−59 |
| Sdc4 | NA | 0.619597869 | 9.47E−59 |
| Ubxn4 | 1.03E−58 | 0.250313647 | 1.03E−58 |
| Ptms | 1.24E−58 | 0.267838974 | 1.24E−58 |
| Wfdc17 | NA | 0.486653465 | 2.57E−58 |
| Gsta2 | 2.63E−58 | 0.252161786 | 2.63E−58 |
| 1110008F13Rik | NA | −0.455270589 | 4.13E−58 |
| Fkbp5 | NA | 0.516128063 | 1.44E−57 |
| mt-Nd6 | NA | −0.477851134 | 1.67E−57 |
| Kctd3 | NA | 0.260833465 | 2.11E−57 |
| Sec61a1 | 2.23E−57 | 0.313944633 | 2.23E−57 |
| Ddx21 | NA | 0.73138069 | 2.25E−57 |
| Pdcd4 | NA | 0.48815423 | 3.64E−57 |
| Upb1 | 7.94E−57 | −0.418695518 | 7.94E−57 |
| Cyp2c37 | NA | −0.886903784 | 9.93E−57 |
| Cd53 | NA | 0.265579528 | 1.37E−56 |
| Park7 | 1.55E−56 | −0.260230015 | 1.55E−56 |
| Hic2 | NA | 0.289767391 | 2.03E−56 |
| Sat1 | NA | 0.68869896 | 2.31E−56 |
| Btg2 | NA | 0.832086378 | 3.81E−56 |
| Usmg5 | 4.55E−56 | −0.43049483 | 4.55E−56 |
| Rgs2 | NA | 0.395729601 | 7.50E−56 |
| Chchd2 | NA | −0.552503492 | 7.88E−56 |
| Slc37a4 | 1.06E−55 | 0.268318739 | 1.06E−55 |
| Inmt | 7.77E−21 | −0.731016404 | 1.21E−55 |
| Cyp2d10 | NA | −0.323151441 | 1.31E−55 |
| Itgb2 | NA | 0.272302012 | 1.36E−55 |
| Cps1 | NA | 0.32450205 | 1.45E−55 |
| Keg1 | 2.32E−52 | −0.824548137 | 1.46E−55 |
| C1qc | NA | 0.586092716 | 2.05E−55 |
| AI182371 | NA | 0.447854829 | 2.62E−55 |
| Rock2 | NA | 0.594094466 | 2.90E−55 |
| Sqstm1 | NA | 0.743150456 | 4.59E−55 |
| Fndc3b | NA | 0.409645989 | 4.69E−55 |
| Uox | NA | −0.553115511 | 6.13E−55 |
| Gulo | NA | −0.801665727 | 6.80E−55 |
| mt-Tq | NA | 0.287889985 | 7.13E−55 |
| C8a | 7.61E−55 | 0.333650228 | 7.61E−55 |
| Tiparp | NA | 0.545567123 | 8.73E−55 |
| Fcgr3 | NA | 0.308247077 | 1.21E−54 |
| Uqcr11 | NA | −0.603079991 | 1.37E−54 |
| Atp5e | NA | −0.650247857 | 2.15E−54 |
| Tram1 | NA | 0.411080257 | 2.39E−54 |
| Prg4 | NA | 0.430643187 | 3.20E−54 |
| Upp2 | NA | −0.69025963 | 3.29E−54 |
| H2-Ab1 | NA | 0.460956687 | 3.42E−54 |
| Gm26917 | NA | 0.823736053 | 5.26E−54 |
| Aldh8a1 | NA | −0.683676902 | 5.66E−54 |
| Glyat | NA | −0.647687911 | 1.11E−53 |
| Msrb1 | NA | −0.59903268 | 1.16E−53 |
| Manf | NA | 0.468881688 | 1.60E−53 |
| Spred1 | NA | 0.388422711 | 1.65E−53 |
| Orm2 | NA | 0.271460545 | 2.71E−53 |
| Sparcl1 | 3.40E−53 | −0.253616986 | 3.40E−53 |
| Cat | NA | 0.262624418 | 7.22E−53 |
| Ndufb11 | 4.56E−22 | −0.509671057 | 8.28E−53 |
| C1qa | NA | 0.514846404 | 1.51E−52 |
| Cldn14 | NA | 0.364540783 | 1.86E−52 |
| Heatr1 | NA | 0.406005371 | 2.23E−52 |
| Calm1 | NA | 0.413146039 | 2.28E−52 |
| Insig1 | NA | 0.465534816 | 3.03E−52 |
| Cfh | 2.09E−34 | 0.400919606 | 3.24E−52 |
| Ppp2r5a | 7.14E−52 | −0.260504538 | 7.14E−52 |
| Hc | NA | 0.367935082 | 1.11E−51 |
| Hebp1 | NA | −0.635432267 | 1.18E−51 |
| Ces1e | 2.36E−51 | 0.265618893 | 2.36E−51 |
| Cox7b | NA | −0.642240776 | 3.42E−51 |
| Abhd2 | NA | 0.484366625 | 1.13E−50 |
| Picalm | NA | 0.754529703 | 1.39E−50 |
| Igfbp1 | NA | 1.058689301 | 1.68E−50 |
| Haao | NA | −0.591181029 | 2.08E−50 |
| Canx | NA | 0.343383207 | 2.09E−50 |
| Cebpb | NA | −0.471579534 | 2.38E−50 |
| Stat3 | NA | 0.715400235 | 2.74E−50 |
| Fgl2 | NA | 0.281793883 | 2.86E−50 |
| AA474408 | NA | 0.43640813 | 3.01E−50 |
| Lyst | NA | 0.409280587 | 8.39E−50 |
| Myo1e | NA | 0.614341001 | 9.34E−50 |
| Tmem87b | NA | 0.434399269 | 1.07E−49 |
| Slc10a2 | NA | 0.299006165 | 2.04E−49 |
| Gnl3 | NA | 0.575970422 | 2.63E−49 |
| Lcn2 | NA | 0.412194792 | 2.72E−49 |
| Jmjd1c | NA | 0.636619821 | 3.43E−49 |
| Cox7a2 | NA | −0.650338386 | 2.00E−48 |
| Cltc | NA | 0.784985399 | 3.05E−48 |
| Rarres2 | NA | −0.552194576 | 3.29E−48 |
| Sc5d | NA | −0.500900917 | 3.57E−48 |
| Ndufa6 | NA | −0.613649978 | 4.43E−48 |
| 2-Mar | NA | −0.55994977 | 4.66E−48 |
| Hspa13 | NA | 0.354844877 | 7.52E−48 |
| Btg1 | NA | 0.458007046 | 1.02E−47 |
| Pah | NA | −0.52207695 | 1.25E−47 |
| Leap2 | NA | −0.671300937 | 1.25E−47 |
| Cytip | NA | 0.267599208 | 1.05E−46 |
| Clpx | NA | −0.404735614 | 1.07E−46 |
| Sdc3 | NA | 0.359804895 | 1.11E−46 |
| mt-Tm | NA | 0.284684642 | 1.20E−46 |
| Gm24187 | NA | 0.431940663 | 1.37E−46 |
| Stk40 | NA | 0.536418312 | 2.15E−46 |
| Cyp2c50 | NA | −0.661823297 | 2.92E−46 |
| Cfp | NA | 0.356178244 | 6.11E−46 |
| Dot1l | NA | 0.397036294 | 7.81E−46 |
| Saa4 | NA | −0.571021905 | 1.16E−45 |
| Atp8b1 | NA | 0.362807422 | 1.42E−45 |
| Mdn1 | NA | 0.317989246 | 1.53E−45 |
| Ndufb2 | 1.66E−45 | −0.577154458 | 1.66E−45 |
| Hsd17b2 | 2.36E−31 | −0.595878526 | 3.18E−45 |
| H2-Eb1 | NA | 0.327872672 | 3.30E−45 |
| Cxadr | NA | 0.698450944 | 4.99E−45 |
| Gstm3 | 5.34E−45 | −0.598573335 | 5.34E−45 |
| Cpb2 | NA | 0.322179327 | 5.47E−45 |
| Spcs3 | NA | 0.394206972 | 9.16E−45 |
| Cd302 | NA | −0.549925078 | 1.10E−44 |
| Plg | NA | 0.261736006 | 1.34E−44 |
| Lactb2 | NA | −0.596505052 | 1.72E−44 |
| Tsr1 | NA | 0.360287403 | 2.32E−44 |
| Igfbp2 | NA | −0.345087789 | 4.02E−44 |
| Rps2 | 5.09E−44 | −0.387862655 | 5.09E−44 |
| Dgkd | NA | 0.370844881 | 1.62E−43 |
| Cml1 | NA | −0.61424052 | 1.70E−43 |
| Sorl1 | NA | 0.306655621 | 1.87E−43 |
| Tra2a | NA | 0.712328169 | 2.35E−43 |
| Dcxr | NA | −0.440851615 | 2.61E−43 |
| Aadac | NA | −0.619958028 | 4.18E−43 |
| Ppib | 6.71E−21 | −0.589699514 | 1.14E−42 |
| Lect2 | NA | −0.626967771 | 1.41E−42 |
| Tlcd2 | NA | −0.594498821 | 1.60E−42 |
| Lpin1 | 1.78E−42 | 0.277494868 | 1.78E−42 |
| Ttc39c | 2.88E−40 | −0.636274928 | 2.03E−42 |
| Tacc2 | NA | 0.329332932 | 3.95E−42 |
| Gchfr | NA | −0.554783473 | 4.46E−42 |
| Tmed7 | NA | 0.386771014 | 6.68E−42 |
| Usp9x | NA | 0.407617259 | 9.98E−42 |
| Aldh1a1 | NA | −0.478797447 | 1.02E−41 |
| Sdad1 | NA | 0.312421192 | 1.30E−41 |
| Fdx1 | NA | −0.623439092 | 1.55E−41 |
| Eef2 | NA | 0.525855561 | 2.71E−41 |
| Gstz1 | NA | −0.464086834 | 2.81E−41 |
| Ptpn1 | NA | 0.41633121 | 3.51E−41 |
| Tnfrsf1a | NA | 0.599573483 | 3.86E−41 |
| Rad54l2 | NA | 0.388705156 | 4.52E−41 |
| Il13ra1 | NA | 0.30296595 | 4.97E−41 |
| Pemt | NA | −0.551184095 | 7.86E−41 |
| Acss2 | 3.19E−11 | −0.620071374 | 8.55E−41 |
| 1-Mar | NA | −0.477762617 | 9.43E−41 |
| Ddx3x | NA | 0.656890035 | 1.07E−40 |
| Hs3st3b1 | NA | 0.343007923 | 1.60E−40 |
| Acox1 | NA | 0.275171428 | 1.78E−40 |
| Atp5j2 | NA | −0.51308051 | 3.32E−40 |
| 8430408G22Rik | NA | −0.50210224 | 4.85E−40 |
| Ndufb9 | NA | −0.503828812 | 5.48E−40 |
| Hif1a | NA | 0.516911277 | 5.71E−40 |
| Hes6 | 1.91E−06 | −0.561302344 | 5.81E−40 |
| Tsku | NA | 0.36762763 | 6.05E−40 |
| Dnajc12 | NA | 0.322094554 | 1.14E−39 |
| Eaf1 | NA | 0.521796522 | 1.19E−39 |
| Qdpr | NA | −0.52357376 | 1.45E−39 |
| Atp5f1 | NA | −0.517061165 | 2.05E−39 |
| Gm4076 | 2.57E−39 | −0.325469418 | 2.57E−39 |
| Uso1 | NA | 0.388069473 | 3.58E−39 |
| Maff | NA | 0.355300257 | 4.89E−39 |
| Qsox1 | NA | 0.342352868 | 9.20E−39 |
| Adh4 | NA | −0.526868306 | 1.92E−38 |
| Chic2 | NA | 0.419790279 | 2.33E−38 |
| Eef1b2 | 2.36E−38 | −0.317323205 | 2.36E−38 |
| Adh5 | NA | −0.491433382 | 2.88E−38 |
| Itih2 | NA | 0.501091194 | 3.43E−38 |
| Gjb2 | NA | 0.60822545 | 3.74E−38 |
| Fabp2 | NA | −0.615569339 | 4.18E−38 |
| Slc20a1 | NA | 0.357248475 | 6.66E−38 |
| Slc3a1 | NA | 0.340997565 | 7.10E−38 |
| Mup2 | 8.16E−38 | 1 | 8.16E−38 |
| Abca1 | NA | 0.724477701 | 8.81E−38 |
| Ndufa7 | NA | −0.555912274 | 1.32E−37 |
| Lgmn | NA | 0.484586871 | 1.80E−37 |
| Gamt | NA | −0.560282685 | 2.41E−37 |
| Bcl3 | NA | 0.289017645 | 3.63E−37 |
| Hint2 | NA | −0.424321102 | 6.90E−37 |
| Mpc2 | NA | −0.482839417 | 7.20E−37 |
| Atxn1 | NA | 0.304881734 | 9.14E−37 |
| Cyp2c54 | 9.37E−37 | −0.39493764 | 9.37E−37 |
| Fos | NA | 0.432003299 | 1.43E−36 |
| St3gal1 | NA | 0.528739882 | 1.81E−36 |
| Msn | NA | 0.260172491 | 1.86E−36 |
| Sds | 3.53E−09 | −0.407006722 | 2.82E−36 |
| Rpn1 | NA | 0.41242919 | 5.16E−36 |
| Ucp2 | NA | 0.261164666 | 8.74E−36 |
| Tcerg1 | NA | 0.357549618 | 1.14E−35 |
| Akr1c20 | NA | −0.554152364 | 1.48E−35 |
| Dst | NA | 0.510971252 | 1.75E−35 |
| Dstn | NA | 0.324301394 | 1.75E−35 |
| S100a1 | NA | −0.376186192 | 1.76E−35 |
| Sar1b | NA | −0.317607513 | 1.79E−35 |
| Crp | NA | −0.506869364 | 2.22E−35 |
| Pfkfb2 | NA | 0.290016291 | 2.41E−35 |
| Gm4952 | NA | −0.49867829 | 4.03E−35 |
| Ndufb6 | NA | −0.507682563 | 4.30E−35 |
| Vsig4 | NA | 0.483290635 | 4.48E−35 |
| Hao1 | NA | 0.32303248 | 4.98E−35 |
| Tubb2a | NA | 0.749433744 | 5.17E−35 |
| Arl4d | NA | −0.507228317 | 6.71E−35 |
| mmu-mir-6236 | 2.00E−10 | 0.787474192 | 7.24E−35 |
| Apobec1 | NA | 0.305082858 | 9.49E−35 |
| Akr1c14 | NA | −0.518570798 | 1.08E−34 |
| Tmem30a | NA | 0.332760453 | 1.95E−34 |
| Cfhr2 | 2.09E−34 | −0.44846316 | 2.09E−34 |
| Slc41a2 | NA | 0.278262888 | 2.13E−34 |
| Ipo7 | NA | 0.324580152 | 2.19E−34 |
| Tsc22d2 | NA | 0.391059638 | 2.43E−34 |
| Cyb5r3 | NA | 0.339033056 | 3.10E−34 |
| Apol7a | NA | −0.492294435 | 3.11E−34 |
| Fxyd1 | NA | −0.479908398 | 3.83E−34 |
| Cox5b | NA | −0.498783616 | 4.68E−34 |
| Rplp1 | NA | −0.504662964 | 4.91E−34 |
| Ugdh | NA | 0.633076584 | 5.17E−34 |
| H2-Aa | NA | 0.327934784 | 6.84E−34 |
| H3f3b | NA | 0.702914634 | 7.03E−34 |
| Ppan | NA | 0.272410087 | 7.48E−34 |
| Ugt2a3 | NA | −0.333441405 | 8.21E−34 |
| Xbp1 | NA | 0.603165814 | 9.59E−34 |
| Tjp2 | NA | 0.411461753 | 1.10E−33 |
| Tatdn2 | NA | 0.373296072 | 1.22E−33 |
| Hibadh | NA | −0.499409672 | 1.26E−33 |
| Pten | NA | 0.355902258 | 1.92E−33 |
| Ctsl | NA | 0.530101805 | 1.93E−33 |
| Hspb8 | NA | 0.633933124 | 2.00E−33 |
| Atp5j | NA | −0.48373528 | 3.59E−33 |
| Usp4 | NA | 0.324732042 | 4.02E−33 |
| Nolc1 | NA | 0.47222269 | 4.11E−33 |
| Ier5 | NA | 0.288994609 | 4.64E−33 |
| Lonp2 | NA | −0.467365882 | 5.23E−33 |
| Rpl32 | 8.16E−20 | −0.532218378 | 1.10E−32 |
| Rrbp1 | NA | 0.306944991 | 1.11E−32 |
| Dio1 | NA | −0.545283744 | 1.11E−32 |
| Tmem256 | NA | −0.518510202 | 1.19E−32 |
| Pdia6 | NA | −0.389966782 | 1.21E−32 |
| Ssr1 | NA | 0.327818552 | 1.30E−32 |
| Gm5096 | NA | −0.363089147 | 1.95E−32 |
| Mcm10 | NA | −0.487427572 | 2.29E−32 |
| Ibtk | NA | 0.331106132 | 4.10E−32 |
| Nit2 | NA | −0.485940613 | 4.49E−32 |
| Ppm1k | NA | −0.558394496 | 6.56E−32 |
| Son | NA | 0.542911509 | 7.13E−32 |
| Adh6-ps1 | NA | −0.418504072 | 9.38E−32 |
| Selenbp1 | NA | −0.493707332 | 9.52E−32 |
| Uggt1 | NA | 0.284457726 | 1.12E−31 |
| Efna1 | NA | 0.336097237 | 1.73E−31 |
| Ssr4 | NA | −0.507577243 | 1.87E−31 |
| Ndufs6 | NA | −0.537929827 | 2.00E−31 |
| 1600014C10Rik | NA | −0.498182953 | 2.43E−31 |
| Gas5 | NA | −0.336153154 | 2.74E−31 |
| Nop56 | NA | 0.265670844 | 2.87E−31 |
| Etfb | NA | −0.365490277 | 4.01E−31 |
| Dusp16 | NA | 0.46051738 | 4.23E−31 |
| Rora | NA | 0.644354953 | 5.02E−31 |
| Nedd4 | NA | 0.377388355 | 6.69E−31 |
| Epb4.1 | NA | 0.562789274 | 1.67E−30 |
| Csde1 | NA | 0.326883793 | 1.68E−30 |
| Ranbp2 | NA | 0.598499679 | 2.18E−30 |
| Kmo | NA | −0.384440188 | 3.26E−30 |
| Ndufa13 | NA | −0.43876131 | 3.68E−30 |
| Rnd3 | NA | 0.36548808 | 3.93E−30 |
| Prkd3 | NA | 0.366517526 | 3.93E−30 |
| Nr5a2 | NA | 0.322039014 | 5.05E−30 |
| Gm23388 | NA | 0.416208382 | 5.50E−30 |
| Ddx5 | NA | 0.522944475 | 5.90E−30 |
| Blvrb | NA | −0.545112721 | 6.23E−30 |
| Lrp1 | NA | 0.505128824 | 6.85E−30 |
| Cfi | NA | 0.251606433 | 7.20E−30 |
| Mmp14 | NA | 0.288913135 | 7.66E−30 |
| Plec | NA | 0.467512335 | 8.30E−30 |
| Rab1 | NA | 0.352627435 | 1.03E−29 |
| Jak1 | NA | 0.603850485 | 1.34E−29 |
| Slco1a1 | 1.36E−29 | −0.362843757 | 1.36E−29 |
| Errfi1 | NA | 0.393680047 | 1.52E−29 |
| Slc38a4 | NA | 0.263698621 | 2.48E−29 |
| Uqcr10 | NA | −0.45190185 | 2.60E−29 |
| Nab1 | NA | 0.33421071 | 3.00E−29 |
| Il6st | NA | 0.333486533 | 3.02E−29 |
| Mpc1 | NA | −0.281762702 | 3.39E−29 |
| Cmah | NA | 0.361552447 | 5.19E−29 |
| Calu | NA | 0.304112196 | 5.69E−29 |
| Mlec | NA | 0.324558531 | 7.94E−29 |
| Tm4sf4 | NA | 0.512552747 | 1.16E−28 |
| Hal | 1.20E−28 | 0.280113531 | 1.20E−28 |
| Cmbl | NA | −0.442706435 | 1.48E−28 |
| Baz1a | NA | 0.259610685 | 1.78E−28 |
| Ak3 | NA | 0.289040998 | 1.86E−28 |
| Nrp1 | NA | 0.360791537 | 1.93E−28 |
| Gpt2 | NA | 0.539114072 | 2.49E−28 |
| Etf1 | NA | 0.559444816 | 2.60E−28 |
| Hook1 | NA | 0.327423989 | 3.21E−28 |
| Ndufb4 | NA | −0.453693039 | 3.64E−28 |
| Lgr4 | NA | 0.268964381 | 9.73E−28 |
| Lrrc59 | NA | 0.377173076 | 1.35E−27 |
| Dkc1 | NA | 0.32575964 | 1.54E−27 |
| Stard10 | NA | −0.338316568 | 1.68E−27 |
| Hpx | NA | −0.303288229 | 1.79E−27 |
| Etnk1 | NA | 0.352068809 | 1.82E−27 |
| Agmat | NA | −0.471832829 | 2.33E−27 |
| Stard4 | NA | 0.351527506 | 2.40E−27 |
| Cyp39a1 | NA | 0.351128984 | 2.44E−27 |
| Malat1 | NA | 0.920628097 | 2.62E−27 |
| Dusp6 | NA | 0.59164596 | 2.71E−27 |
| Ywhag | NA | 0.58241474 | 3.05E−27 |
| Adipor2 | NA | −0.473791512 | 3.14E−27 |
| Arcn1 | NA | 0.327775216 | 3.29E−27 |
| Serp1 | 1.29E−19 | 0.312399212 | 3.50E−27 |
| Ifitm3 | 4.18E−27 | −0.425091425 | 4.18E−27 |
| Ces3b | 5.30E−27 | −0.36637965 | 5.30E−27 |
| Fcna | NA | 0.377428055 | 5.71E−27 |
| App | NA | 0.361832458 | 6.12E−27 |
| Cyp2c67 | NA | −0.463488581 | 1.02E−26 |
| Lrrfip1 | NA | 0.445928709 | 1.19E−26 |
| Dhrs3 | NA | −0.467621234 | 1.25E−26 |
| Rpl36a1 | NA | −0.475541391 | 1.26E−26 |
| Cml2 | NA | −0.397916138 | 1.85E−26 |
| Ppp1r3b | NA | −0.402657788 | 2.29E−26 |
| Eif4g1 | NA | 0.374860037 | 2.42E−26 |
| Ptpn12 | NA | 0.33417878 | 5.69E−26 |
| Pik3c2a | NA | 0.324870962 | 5.98E−26 |
| Cyp4a12a | 7.00E−24 | −0.456827492 | 6.69E−26 |
| Mug2 | NA | 0.370406564 | 6.96E−26 |
| Lman1 | NA | 0.29860668 | 9.00E−26 |
| Hspe1 | 1.46E−15 | −0.480734946 | 9.37E−26 |
| Mrpl14 | NA | −0.459040925 | 9.74E−26 |
| Zfp683 | NA | −0.421689112 | 1.05E−25 |
| Mak16 | NA | 0.317126116 | 1.09E−25 |
| Acsl4 | NA | 0.314743242 | 1.14E−25 |
| Copa | NA | 0.345094339 | 1.16E−25 |
| mt-Atp6 | 3.18E−23 | −0.573743033 | 1.31E−25 |
| Uqcrh | NA | −0.429164519 | 1.41E−25 |
| Slc38a3 | NA | −0.40978981 | 1.45E−25 |
| Minos1 | NA | −0.455548025 | 2.00E−25 |
| Ebpl | NA | −0.450334658 | 2.09E−25 |
| Atox1 | NA | −0.446203219 | 2.48E−25 |
| Tbc1d15 | NA | 0.488646353 | 2.52E−25 |
| Aox3 | NA | −0.333319828 | 2.60E−25 |
| Map2k3 | NA | 0.434654367 | 2.73E−25 |
| Vkorc1 | NA | −0.404176916 | 3.15E−25 |
| Cyp7a1 | NA | −0.431201844 | 3.19E−25 |
| Trip12 | NA | 0.328304235 | 3.31E−25 |
| Hpgd | NA | −0.387752401 | 3.49E−25 |
| Lmo4 | NA | 0.320534245 | 3.97E−25 |
| Cyb5b | NA | −0.423345561 | 4.82E−25 |
| Klf15 | NA | −0.451831953 | 5.02E−25 |
| Gnl2 | NA | 0.390473036 | 5.63E−25 |
| 3110043O21Rik | NA | 0.269386631 | 5.64E−25 |
| Asgr1 | NA | −0.385655364 | 6.52E−25 |
| Mtus1 | NA | 0.362060296 | 7.17E−25 |
| Cyp2j5 | NA | −0.398305774 | 7.33E−25 |
| Mup1 | 7.40E−25 | 1 | 7.40E−25 |
| Prdx5 | NA | −0.28208825 | 8.22E−25 |
| Chd1 | NA | 0.378763061 | 8.51E−25 |
| Rarres1 | 1.03E−20 | −0.455632451 | 8.53E−25 |
| Sel1l | NA | 0.33595882 | 8.76E−25 |
| Arl5a | NA | 0.264805346 | 1.52E−24 |
| Mpeg1 | NA | 0.493478112 | 1.80E−24 |
| Wdr43 | NA | 0.343830267 | 1.89E−24 |
| Aff4 | NA | 0.616309327 | 1.96E−24 |
| Timm13 | NA | −0.432961802 | 2.46E−24 |
| 1500017E21Rik | NA | −0.460997215 | 2.47E−24 |
| Nop10 | NA | −0.291908137 | 2.62E−24 |
| Serpina12 | 2.90E−24 | −0.453399356 | 2.90E−24 |
| Fam107b | NA | 0.463320341 | 2.90E−24 |
| Cep85l | NA | 0.277893187 | 3.24E−24 |
| Srpr | NA | 0.305179041 | 4.78E−24 |
| Narf | NA | 0.262933124 | 6.15E−24 |
| 2410015M20Rik | NA | −0.402123463 | 6.84E−24 |
| Gtpbp4 | NA | 0.482239467 | 6.84E−24 |
| Prdx4 | 8.80E−10 | −0.417588892 | 6.96E−24 |
| Ccs | NA | −0.426694207 | 7.67E−24 |
| Rps15 | NA | −0.455155041 | 7.79E−24 |
| Tns1 | NA | 0.434939232 | 8.90E−24 |
| Esd | NA | −0.280760023 | 9.90E−24 |
| Ncl | NA | 0.48450982 | 1.05E−23 |
| Stom | NA | 0.323229029 | 1.13E−23 |
| Amacr | NA | −0.413582585 | 1.14E−23 |
| Sco2 | NA | −0.292870629 | 1.32E−23 |
| Glo1 | NA | −0.406574019 | 1.87E−23 |
| Mcl1 | NA | 0.570934356 | 2.05E−23 |
| Mybbp1a | NA | 0.431161557 | 2.11E−23 |
| Nars | NA | 0.437769953 | 2.87E−23 |
| Slc38a10 | NA | 0.285548034 | 2.98E−23 |
| Gm10800 | NA | 0.562597493 | 3.19E−23 |
| Stt3b | NA | 0.319405267 | 3.84E−23 |
| Srp72 | NA | 0.310685136 | 5.31E−23 |
| Prrc2c | NA | 0.439271845 | 6.04E−23 |
| Zyx | NA | 0.298338034 | 6.24E−23 |
| Sc4mol | NA | −0.326170476 | 6.26E−23 |
| Mrp63 | NA | −0.442201567 | 7.79E−23 |
| Arg1 | NA | 0.317948822 | 8.62E−23 |
| Arhgef12 | NA | 0.590764816 | 8.83E−23 |
| Acsm1 | NA | −0.369245009 | 9.63E−23 |
| Mat2a | NA | 0.482570924 | 1.45E−22 |
| Cars | NA | 0.315814572 | 1.93E−22 |
| Rps9 | NA | −0.371793799 | 1.94E−22 |
| Ell2 | NA | 0.417241283 | 2.55E−22 |
| Gm24245 | NA | 0.546463773 | 3.25E−22 |
| Rpl13a | NA | −0.381462137 | 3.58E−22 |
| Bzw1 | NA | 0.294810535 | 3.59E−22 |
| Grlf1 | NA | 0.295046691 | 3.99E−22 |
| Bbox1 | NA | −0.408245616 | 4.17E−22 |
| Map3k5 | NA | 0.33471162 | 4.76E−22 |
| Pecr | NA | −0.398084036 | 4.93E−22 |
| Ndufa3 | NA | −0.41160475 | 5.38E−22 |
| Tmem14c | NA | −0.368514287 | 6.04E−22 |
| Atp13a3 | NA | 0.394480252 | 6.61E−22 |
| Pxk | NA | 0.281854648 | 6.95E−22 |
| Rps18 | NA | −0.488540797 | 8.18E−22 |
| Mrps24 | NA | −0.390380395 | 8.75E−22 |
| Mtss1 | NA | 0.287581972 | 1.08E−21 |
| Gjb1 | NA | −0.31656535 | 1.13E−21 |
| Edem1 | NA | 0.286881059 | 1.13E−21 |
| Spp2 | NA | −0.382096801 | 1.28E−21 |
| Kif5b | NA | 0.50116686 | 1.57E−21 |
| Acly | NA | 0.328803101 | 1.74E−21 |
| Rpl13 | NA | −0.299693639 | 1.78E−21 |
| 1110001J03Rik | NA | −0.432785224 | 2.04E−21 |
| Dync1li1 | NA | 0.262406206 | 2.23E−21 |
| Ndufa2 | NA | −0.396473631 | 2.46E−21 |
| Txndc5 | NA | 0.290124872 | 3.56E−21 |
| Psme1 | NA | −0.394006165 | 3.58E−21 |
| Ppp1r12a | NA | 0.356295305 | 3.60E−21 |
| Bphl | NA | −0.40747923 | 5.02E−21 |
| Trf | NA | −0.261883505 | 5.32E−21 |
| Cyp2c68 | NA | −0.398830868 | 7.05E−21 |
| Trib1 | NA | 0.331693479 | 7.49E−21 |
| Akr1c19 | NA | −0.272667086 | 7.78E−21 |
| Cpne3 | NA | 0.258007443 | 8.12E−21 |
| Pnpla7 | 2.65E−15 | −0.301626901 | 1.01E−20 |
| Dnajb11 | NA | 0.315357308 | 1.04E−20 |
| Phc2 | NA | 0.31203626 | 1.14E−20 |
| Atp6v1f | NA | −0.252893521 | 1.18E−20 |
| Tars | NA | 0.492059498 | 1.28E−20 |
| Ctnnb1 | NA | 0.295681078 | 1.58E−20 |
| Id3 | NA | −0.351088732 | 1.84E−20 |
| Tmem243 | NA | −0.379053275 | 2.35E−20 |
| Eci1 | NA | −0.276383144 | 2.48E−20 |
| Lap3 | NA | −0.364371999 | 2.65E−20 |
| Cebpa | NA | −0.39155301 | 3.89E−20 |
| Tfpi2 | NA | −0.366342462 | 4.58E−20 |
| Il6ra | NA | 0.304392668 | 5.08E−20 |
| Cald1 | NA | 0.373703655 | 5.69E−20 |
| Neat1 | NA | 0.63740381 | 5.72E−20 |
| Ptprf | NA | 0.307126385 | 6.50E−20 |
| Uqcrb | NA | −0.408416744 | 6.59E−20 |
| Ostc | NA | 0.265829876 | 7.22E−20 |
| Sec31a | NA | 0.263042758 | 7.68E−20 |
| Mbnl1 | NA | 0.451258107 | 9.50E−20 |
| Osbpl9 | NA | 0.278773264 | 1.04E−19 |
| Alad | NA | −0.420641438 | 1.16E−19 |
| Gch1 | NA | −0.282499843 | 1.62E−19 |
| Lrrfip2 | NA | 0.373099277 | 1.85E−19 |
| Ireb2 | NA | 0.265741209 | 1.87E−19 |
| Cisd1 | NA | −0.372694857 | 2.37E−19 |
| Mocs2 | NA | −0.346681141 | 2.62E−19 |
| Coa6 | NA | −0.411409685 | 2.73E−19 |
| Mrc1 | NA | 0.340444916 | 2.90E−19 |
| Copb2 | NA | 0.283928956 | 3.01E−19 |
| Stk17b | NA | 0.291050054 | 3.56E−19 |
| Ghr | NA | −0.364775879 | 3.66E−19 |
| Ptp4a1 | NA | 0.262691158 | 3.82E−19 |
| Osbpl1a | NA | 0.272788799 | 4.18E−19 |
| Atp5g3 | NA | −0.300221819 | 4.43E−19 |
| Cyp27a1 | NA | −0.373510332 | 4.44E−19 |
| Akr1a1 | NA | −0.326935362 | 4.63E−19 |
| Atp5c1 | NA | −0.347648538 | 5.04E−19 |
| Nme1 | 4.26E−14 | −0.432885306 | 5.43E−19 |
| Azin1 | NA | 0.270139699 | 6.16E−19 |
| Purb | NA | 0.35248294 | 6.99E−19 |
| Timm8b | NA | −0.393581904 | 8.39E−19 |
| Uhrf1bp1l | NA | 0.419802385 | 8.66E−19 |
| Sfpq | NA | 0.393977901 | 9.08E−19 |
| Gsk3b | NA | 0.406458835 | 9.12E−19 |
| Iqgap2 | NA | 0.254698841 | 9.72E−19 |
| Cideb | NA | −0.368365389 | 1.02E−18 |
| Ndufc2 | NA | −0.425284817 | 1.02E−18 |
| Palld | NA | 0.341357023 | 1.34E−18 |
| D10Jhu81e | NA | −0.395730917 | 1.93E−18 |
| Pxmp2 | NA | −0.367129885 | 2.40E−18 |
| 15-Sep | NA | −0.409167133 | 2.53E−18 |
| Smg1 | NA | 0.334990596 | 2.85E−18 |
| Aass | NA | 0.477517138 | 3.07E−18 |
| Hnrnpdl | NA | 0.397994117 | 3.09E−18 |
| Akap11 | NA | 0.346362286 | 4.54E−18 |
| Nedd4l | NA | 0.281232817 | 4.90E−18 |
| Jhdm1d | NA | 0.304101624 | 4.92E−18 |
| 1500011K16Rik | NA | −0.329639846 | 5.16E−18 |
| Rps3 | NA | −0.349320751 | 5.25E−18 |
| Slc22a30 | 1.34E−16 | −0.374598404 | 5.26E−18 |
| Erp44 | NA | 0.279450299 | 5.51E−18 |
| Coro1c | NA | 0.306679199 | 6.74E−18 |
| Ptpn2 | NA | 0.273418437 | 7.37E−18 |
| Commd6 | NA | −0.364529559 | 7.88E−18 |
| Fitm1 | NA | −0.324153911 | 7.90E−18 |
| Dgat2 | NA | −0.343266251 | 8.35E−18 |
| Surf4 | NA | 0.258577589 | 9.74E−18 |
| Gm10801 | NA | 0.265934225 | 1.30E−17 |
| Cox6b1 | NA | −0.362017449 | 1.47E−17 |
| Adtrp | NA | −0.366413277 | 1.53E−17 |
| Ankrd11 | NA | 0.415626852 | 1.70E−17 |
| Ccbl1 | NA | −0.36488302 | 2.18E−17 |
| Ftl1 | 1 | 1 | 2.43E−17 |
| Prkaa2 | NA | 0.263640766 | 2.46E−17 |
| Arl6ip1 | NA | −0.364945533 | 2.55E−17 |
| Egr1 | NA | 0.584558888 | 2.80E−17 |
| Dnajb9 | NA | 0.25864192 | 2.89E−17 |
| Smdt1 | NA | −0.367317155 | 3.21E−17 |
| Ddc | NA | −0.360752783 | 3.48E−17 |
| Ndufc1 | NA | −0.344432714 | 3.78E−17 |
| Ndufb8 | NA | −0.348076577 | 3.81E−17 |
| Zbtb20 | NA | 0.471532978 | 4.06E−17 |
| Xiap | NA | 0.26016306 | 4.75E−17 |
| Tjp1 | NA | 0.286163863 | 4.89E−17 |
| Anp32a | NA | −0.316957742 | 5.23E−17 |
| Ide | NA | 0.251691529 | 5.60E−17 |
| Ehhadh | NA | −0.250472633 | 7.29E−17 |
| Sptbn1 | NA | 0.42268289 | 7.32E−17 |
| Mbl2 | NA | −0.346518253 | 7.59E−17 |
| Kpnb1 | NA | 0.280415627 | 7.91E−17 |
| Mien1 | NA | −0.370437762 | 8.08E−17 |
| Gne | NA | −0.425142761 | 9.04E−17 |
| Bola3 | NA | −0.368862428 | 1.08E−16 |
| Akap13 | NA | 0.495253767 | 1.14E−16 |
| Hist1h1c | NA | −0.300906532 | 1.17E−16 |
| Nr2f6 | NA | −0.375433952 | 1.37E−16 |
| Ces1f | NA | −0.312589958 | 1.45E−16 |
| Ndufv3 | NA | −0.397145193 | 1.45E−16 |
| Cast | NA | 0.400886525 | 1.50E−16 |
| Golga4 | NA | 0.352084678 | 1.56E−16 |
| Apom | NA | −0.302410414 | 1.68E−16 |
| Ebp | NA | −0.355328266 | 1.79E−16 |
| Gpam | NA | 0.250377871 | 1.86E−16 |
| Edf1 | NA | −0.381679393 | 1.87E−16 |
| Srebf1 | NA | 0.27619787 | 1.90E−16 |
| Ndufs8 | NA | −0.361439192 | 1.93E−16 |
| Eprs | NA | 0.37008595 | 1.94E−16 |
| Ndufa1 | NA | −0.364515751 | 1.96E−16 |
| Litaf | NA | 0.394366845 | 2.04E−16 |
| H6pd | NA | 0.343718187 | 2.22E−16 |
| Bptf | NA | 0.324463857 | 2.66E−16 |
| Ifrd1 | NA | 0.490389826 | 2.92E−16 |
| Plk3 | NA | 0.416624571 | 3.12E−16 |
| Nadk2 | NA | 0.257852312 | 3.23E−16 |
| Myl12a | NA | 0.390780681 | 3.32E−16 |
| Psmb1 | NA | −0.358482353 | 3.95E−16 |
| Baat | NA | −0.308582001 | 4.31E−16 |
| Cnpy2 | NA | −0.372850093 | 4.94E−16 |
| Bhmt2 | NA | −0.311413339 | 5.24E−16 |
| Gstm4 | NA | −0.380861133 | 5.61E−16 |
| Chac2 | NA | −0.286511682 | 6.00E−16 |
| Mettl20 | NA | −0.331367185 | 6.50E−16 |
| Bckdha | NA | −0.349979609 | 6.82E−16 |
| BC089597 | NA | 0.262237515 | 6.90E−16 |
| Sirt3 | NA | −0.345997558 | 7.30E−16 |
| Mdm2 | NA | 0.328296258 | 8.15E−16 |
| Acy3 | NA | −0.313748988 | 8.20E−16 |
| Hsp90ab1 | NA | 0.322748941 | 8.34E−16 |
| Mrpl12 | NA | −0.308200176 | 9.04E−16 |
| Fam120a | NA | 0.280043634 | 9.81E−16 |
| Atp5o | NA | −0.331628292 | 9.93E−16 |
| Fkbp4 | NA | −0.353366286 | 1.01E−15 |
| Cldn3 | NA | −0.314469359 | 1.08E−15 |
| Ipo5 | NA | 0.268735631 | 1.12E−15 |
| Fpgs | NA | −0.34940945 | 1.15E−15 |
| Sdsl | NA | 0.259290764 | 1.41E−15 |
| Tgm2 | NA | 0.399330712 | 1.43E−15 |
| Phlda1 | NA | 0.549174347 | 1.46E−15 |
| Macf1 | NA | 0.404177263 | 1.47E−15 |
| Mrps18c | NA | −0.366291099 | 1.60E−15 |
| Sbno1 | NA | 0.367100512 | 1.62E−15 |
| Nedd8 | NA | −0.356380686 | 1.86E−15 |
| Magt1 | NA | 0.263295188 | 1.95E−15 |
| Sdhb | NA | −0.289642823 | 2.09E−15 |
| Ethe1 | NA | −0.333417645 | 2.45E−15 |
| Mdh1 | NA | −0.307657821 | 3.99E−15 |
| Nrip1 | NA | 0.272854584 | 4.11E−15 |
| Lcp1 | NA | 0.484011001 | 4.22E−15 |
| Srsf11 | NA | 0.401209055 | 4.47E−15 |
| Shb | NA | 0.315236194 | 4.68E−15 |
| Bsg | NA | −0.31515012 | 5.31E−15 |
| Hibch | NA | −0.327130099 | 6.26E−15 |
| Mki67ip | NA | 0.318372964 | 6.27E−15 |
| Otc | NA | −0.29903416 | 6.99E−15 |
| Smap2 | NA | 0.348911433 | 9.52E−15 |
| Nr0b2 | NA | −0.293010319 | 1.07E−14 |
| Pank1 | NA | −0.352650118 | 1.09E−14 |
| Ak2 | NA | −0.345102135 | 1.13E−14 |
| Rlim | NA | 0.31023282 | 1.42E−14 |
| Erbb2ip | NA | 0.333163506 | 1.69E−14 |
| Huwe1 | NA | 0.387931325 | 1.75E−14 |
| Hmgcs1 | NA | −0.349112403 | 1.98E−14 |
| Rps21 | NA | −0.364450868 | 2.02E−14 |
| Grhpr | NA | −0.353058662 | 2.13E−14 |
| Tm7sf2 | NA | −0.316381125 | 2.28E−14 |
| Txn1 | NA | −0.309123787 | 2.50E−14 |
| Sh3bgrl | NA | 0.25128227 | 2.71E−14 |
| 0610011F06Rik | NA | −0.303688511 | 2.80E−14 |
| Psmb6 | NA | −0.319377533 | 2.87E−14 |
| Gm2788 | NA | −0.283138796 | 2.89E−14 |
| AW112010 | NA | −0.298104828 | 3.17E−14 |
| Dhdh | NA | −0.334814135 | 4.04E−14 |
| Sf1 | NA | 0.30230624 | 4.12E−14 |
| Tbl1xr1 | NA | 0.307509115 | 4.15E−14 |
| Coq9 | NA | −0.347936449 | 4.20E−14 |
| Il4ra | NA | 0.25739098 | 4.44E−14 |
| Tceb2 | NA | −0.32073626 | 4.50E−14 |
| Rps10 | NA | −0.33690905 | 4.59E−14 |
| Psme4 | NA | 0.262584214 | 4.62E−14 |
| Gm4951 | NA | −0.322087312 | 5.37E−14 |
| Pak2 | NA | 0.281467155 | 5.57E−14 |
| Metap2 | NA | 0.368604828 | 6.59E−14 |
| 2010107E04Rik | NA | −0.318247806 | 7.21E−14 |
| Prox1 | NA | 0.266329524 | 7.66E−14 |
| Ddx6 | NA | 0.444275069 | 7.70E−14 |
| Pacsin2 | NA | 0.370825053 | 7.89E−14 |
| Hdac11 | NA | −0.33684651 | 8.63E−14 |
| Ubl5 | NA | −0.303313218 | 1.12E−13 |
| Mettl7a1 | NA | −0.322098553 | 1.21E−13 |
| Larp4b | NA | 0.34214672 | 1.22E−13 |
| Uqcc2 | NA | −0.326858302 | 1.27E−13 |
| 1810022K09Rik | NA | −0.300081452 | 1.29E−13 |
| Selk | 1.30E−13 | −0.338314398 | 1.30E−13 |
| Dhx9 | NA | 0.350364065 | 1.41E−13 |
| Psmb3 | NA | −0.330302519 | 1.47E−13 |
| Rexo2 | NA | −0.3263129 | 1.48E−13 |
| Hipk3 | NA | 0.339385214 | 1.58E−13 |
| Cyp4f14 | NA | −0.295933211 | 1.60E−13 |
| Wdr26 | NA | 0.432510772 | 1.94E−13 |
| Sec61b | NA | −0.290284119 | 2.20E−13 |
| Paqr9 | NA | −0.332341635 | 2.23E−13 |
| Tsc22d1 | NA | −0.299728636 | 2.38E−13 |
| Glyctk | NA | −0.297701904 | 2.68E−13 |
| Mapk6 | NA | 0.256831906 | 3.22E−13 |
| Atp1b1 | NA | 0.370203015 | 3.27E−13 |
| Aqp9 | NA | −0.310863805 | 3.38E−13 |
| Suco | NA | 0.250962583 | 3.39E−13 |
| Dync1h1 | NA | 0.379607116 | 4.50E−13 |
| Ctsb | NA | 0.521310448 | 4.83E−13 |
| Tpr | NA | 0.440921219 | 5.22E−13 |
| Supt6 | NA | 0.291294768 | 5.48E−13 |
| Lsmd1 | NA | −0.313140806 | 5.95E−13 |
| Kif21a | NA | 0.404170578 | 6.46E−13 |
| Sgk1 | NA | 0.284303028 | 7.18E−13 |
| Proz | NA | 0.263204472 | 7.77E−13 |
| Romo1 | NA | −0.335854841 | 7.99E−13 |
| Cfhr1 | NA | −0.325332852 | 8.01E−13 |
| Deptor | NA | 0.260618372 | 8.58E−13 |
| Atp2b1 | NA | 0.332096305 | 9.01E−13 |
| Rnf13 | NA | 0.255296715 | 9.16E−13 |
| Dhrs4 | NA | −0.332074421 | 9.59E−13 |
| Lasp1 | NA | 0.253021439 | 9.93E−13 |
| Tmem147 | NA | −0.283785217 | 1.00E−12 |
| Fus | NA | 0.339324351 | 1.03E−12 |
| Yap1 | NA | 0.328109852 | 1.07E−12 |
| Acads | NA | −0.32421998 | 1.10E−12 |
| Fam25c | NA | −0.297673676 | 1.12E−12 |
| Ndufb3 | NA | −0.327185415 | 1.16E−12 |
| Agpat2 | NA | −0.298159013 | 1.22E−12 |
| Bst2 | NA | −0.315565973 | 1.52E−12 |
| Caprin1 | NA | 0.262825966 | 1.56E−12 |
| Syncrip | NA | 0.27476476 | 1.65E−12 |
| Rnaseh2c | NA | −0.325683719 | 1.67E−12 |
| Cldn2 | NA | −0.251076838 | 1.86E−12 |
| Klf3 | NA | 0.267357487 | 1.89E−12 |
| Tnks2 | NA | 0.317626402 | 1.91E−12 |
| Derl2 | NA | −0.331734152 | 2.28E−12 |
| Ndufv2 | NA | −0.327639406 | 2.48E−12 |
| Eif4b | NA | 0.36816236 | 2.53E−12 |
| Cyp4f15 | NA | −0.299840345 | 2.53E−12 |
| Brd2 | NA | 0.571515166 | 2.65E−12 |
| Copz1 | NA | 0.257722711 | 2.65E−12 |
| Ddx27 | NA | 0.252155421 | 3.46E−12 |
| Mcee | NA | −0.31991829 | 3.52E−12 |
| Dnajc19 | NA | −0.316006732 | 3.54E−12 |
| Aldh4a1 | NA | −0.29070878 | 3.75E−12 |
| Fdps | NA | −0.266347718 | 4.58E−12 |
| Reep3 | NA | 0.409014614 | 5.12E−12 |
| Car5a | NA | −0.308769058 | 5.42E−12 |
| Tmem37 | NA | −0.282207793 | 6.77E−12 |
| Tspo | NA | −0.315729205 | 6.95E−12 |
| Tspan31 | NA | −0.295881364 | 7.18E−12 |
| Atp11c | NA | 0.260536506 | 7.22E−12 |
| Atf6 | NA | 0.377819844 | 7.66E−12 |
| Rpl14 | NA | −0.277637754 | 7.87E−12 |
| Med13 | NA | 0.294918978 | 7.95E−12 |
| Sdr9c7 | NA | −0.259988635 | 8.11E−12 |
| Rps8 | 8.37E−12 | −0.274601282 | 8.37E−12 |
| Kpna1 | NA | 0.32304803 | 8.67E−12 |
| Btaf1 | NA | 0.275463861 | 8.92E−12 |
| Ddah1 | NA | −0.312000429 | 9.85E−12 |
| Ndufs4 | NA | −0.313160933 | 1.01E−11 |
| Fam214a | NA | −0.265769677 | 1.02E−11 |
| Rbm25 | NA | 0.421459678 | 1.03E−11 |
| Ndufb10 | NA | −0.300358086 | 1.04E−11 |
| Slirp | NA | −0.308519262 | 1.04E−11 |
| Scaf11 | NA | 0.359845436 | 1.30E−11 |
| Anapc13 | NA | −0.364181956 | 1.31E−11 |
| Arpc1b | NA | 0.259676842 | 1.36E−11 |
| Pnkd | NA | −0.292772175 | 1.41E−11 |
| Abcd3 | NA | −0.271805105 | 1.44E−11 |
| Hmgcr | NA | 0.416711628 | 1.61E−11 |
| Mxd1 | NA | 0.297767874 | 1.65E−11 |
| Bet1 | NA | −0.296563627 | 1.73E−11 |
| Cela1 | NA | −0.272296838 | 1.78E−11 |
| Tmem160 | NA | −0.339044834 | 2.24E−11 |
| Snx10 | NA | 0.285642594 | 2.35E−11 |
| Ubb | 0.120646029 | −0.275030382 | 2.58E−11 |
| Lgals9 | NA | −0.269998113 | 2.87E−11 |
| Aadat | NA | −0.318627951 | 3.04E−11 |
| Mrpl33 | NA | −0.265447876 | 3.06E−11 |
| Tmem258 | NA | −0.27689533 | 3.18E−11 |
| Cyp2d40 | NA | −0.321717407 | 3.46E−11 |
| Birc6 | NA | 0.344367807 | 3.56E−11 |
| Naprt1 | NA | −0.338143058 | 4.15E−11 |
| Agxt | NA | −0.258383387 | 4.28E−11 |
| Kdelr2 | NA | 0.397729248 | 4.46E−11 |
| Stbd1 | NA | −0.273216024 | 5.39E−11 |
| Phf20l1 | NA | 0.308488005 | 5.49E−11 |
| Tmprss6 | NA | −0.280234101 | 5.62E−11 |
| Gstk1 | NA | −0.282802747 | 5.77E−11 |
| Gadd45gip1 | NA | −0.304090556 | 6.25E−11 |
| Ubr4 | NA | 0.330144527 | 7.63E−11 |
| Sigmar1 | NA | −0.305468332 | 7.64E−11 |
| Ythdc1 | NA | 0.264130576 | 8.40E−11 |
| Psma6 | NA | −0.30353658 | 8.48E−11 |
| Acaa1a | NA | −0.301848759 | 9.21E−11 |
| Cyp2c55 | NA | −0.290435372 | 9.36E−11 |
| Proc | NA | −0.270719362 | 9.66E−11 |
| Mrpl20 | NA | −0.309348635 | 1.03E−10 |
| Myeov2 | NA | −0.305189789 | 1.05E−10 |
| Dnajc5 | NA | 0.326483514 | 1.16E−10 |
| Kdm5a | NA | 0.351390096 | 1.27E−10 |
| Commd3 | NA | −0.265556375 | 1.46E−10 |
| Mrpl42 | NA | −0.284507519 | 1.47E−10 |
| Bdhl | NA | −0.293611347 | 1.50E−10 |
| Mrpl15 | NA | −0.291228504 | 1.56E−10 |
| Abcb11 | NA | −0.261778471 | 1.57E−10 |
| Ddost | NA | 0.250356033 | 1.89E−10 |
| Anxa5 | NA | 0.365372347 | 2.08E−10 |
| Gsr | NA | 0.412829579 | 2.10E−10 |
| Atf4 | NA | 0.341134734 | 2.33E−10 |
| Iah1 | NA | −0.292985785 | 2.41E−10 |
| Pdzd8 | NA | 0.286270568 | 2.46E−10 |
| Fbxo21 | NA | −0.263087536 | 2.85E−10 |
| Ftsj3 | NA | 0.255227431 | 2.86E−10 |
| C6 | 3.03E−10 | −0.26698766 | 3.03E−10 |
| Acot13 | NA | −0.288638646 | 3.05E−10 |
| Acat3 | NA | −0.311510623 | 3.16E−10 |
| Magi3 | NA | 0.269961841 | 3.24E−10 |
| Cybb | NA | 0.377799564 | 3.48E−10 |
| Tmem242 | NA | −0.272538433 | 3.57E−10 |
| Mrpl41 | NA | −0.267412137 | 3.73E−10 |
| Sema4g | NA | −0.250459516 | 3.89E−10 |
| Cap1 | NA | 0.325055169 | 4.46E−10 |
| Csrp3 | NA | −0.293996249 | 4.47E−10 |
| Hpn | NA | −0.268601326 | 4.81E−10 |
| Slc25a51 | NA | 0.263985158 | 6.14E−10 |
| Spcs1 | NA | −0.26287037 | 6.30E−10 |
| Apof | NA | −0.252173807 | 6.73E−10 |
| Cep85 | NA | 0.28389234 | 6.87E−10 |
| Aes | NA | −0.264214993 | 7.53E−10 |
| Pet100 | NA | −0.28003555 | 7.60E−10 |
| Gcat | NA | −0.258728349 | 7.71E−10 |
| Rps24 | NA | −0.262473665 | 8.21E−10 |
| Dcaf11 | NA | −0.281659388 | 9.55E−10 |
| Pmpcb | NA | −0.263615722 | 1.03E−09 |
| Lipc | NA | −0.254841484 | 1.09E−09 |
| Ptpn11 | NA | 0.277804552 | 1.15E−09 |
| Ccdc50 | NA | 0.285830087 | 1.18E−09 |
| Ivns1abp | NA | 0.356610399 | 1.20E−09 |
| Nqo2 | NA | −0.267802053 | 1.22E−09 |
| Abcg2 | NA | −0.280923445 | 1.38E−09 |
| Dera | NA | −0.267441985 | 1.43E−09 |
| Fasn | NA | 0.291406736 | 1.44E−09 |
| Rabac1 | NA | −0.265987205 | 1.45E−09 |
| Gm20594 | 1.55E−09 | −0.360000649 | 1.55E−09 |
| Clic4 | NA | 0.299795775 | 1.64E−09 |
| Ugt3a2 | NA | −0.258613351 | 1.89E−09 |
| Mvd | NA | −0.270409867 | 1.91E−09 |
| Ptpmt1 | NA | −0.255981462 | 2.02E−09 |
| 0610007P14Rik | NA | −0.257015045 | 2.31E−09 |
| Swi5 | NA | −0.262086804 | 2.33E−09 |
| Tpi1 | NA | −0.26157943 | 2.35E−09 |
| Plcxd2 | NA | −0.275192522 | 2.48E−09 |
| Tmem150a | NA | −0.253507791 | 2.61E−09 |
| Rnf169 | NA | 0.333974467 | 2.63E−09 |
| Deb1 | NA | −0.267029156 | 2.74E−09 |
| Hsbp1 | NA | −0.286726926 | 2.99E−09 |
| Slc26a1 | NA | −0.28165635 | 3.01E−09 |
| Rsl1d1 | NA | 0.346056141 | 3.82E−09 |
| Map3k2 | NA | 0.251791147 | 3.83E−09 |
| Abcf1 | NA | 0.303190272 | 4.09E−09 |
| Psma1 | NA | −0.256671259 | 4.64E−09 |
| Fam195a | NA | −0.264890901 | 4.66E−09 |
| Mrpl11 | NA | −0.268482417 | 4.92E−09 |
| Selo | NA | −0.257421903 | 5.24E−09 |
| Plbd2 | NA | 0.264873115 | 5.33E−09 |
| Rap1b | NA | 0.340766029 | 6.89E−09 |
| Ppip5k2 | NA | 0.320939415 | 7.41E−09 |
| Pcm1 | NA | 0.265397644 | 8.07E−09 |
| Acsm5 | NA | −0.261394035 | 8.53E−09 |
| Rangap1 | NA | 0.296226768 | 1.08E−08 |
| Rraga | NA | 0.262613483 | 1.10E−08 |
| 1110058L19Rik | NA | −0.258088587 | 1.12E−08 |
| Dsp | NA | 0.265533344 | 1.15E−08 |
| Ccnl1 | NA | 0.256623048 | 1.44E−08 |
| Chmp4b | NA | 0.300153098 | 1.52E−08 |
| Ubr5 | NA | 0.305718363 | 1.72E−08 |
| Net1 | NA | 0.34785505 | 1.84E−08 |
| H2-D1 | NA | 0.286522764 | 2.31E−08 |
| Srd5a1 | NA | −0.277347272 | 2.35E−08 |
| Papss2 | NA | −0.254475327 | 2.73E−08 |
| Dnaja2 | NA | 0.372342331 | 2.79E−08 |
| Srrm2 | NA | 0.328159189 | 3.12E−08 |
| Herc1 | NA | 0.256188826 | 3.26E−08 |
| Gtf2a2 | NA | 0.279207862 | 3.58E−08 |
| Pkp4 | NA | 0.323033636 | 4.02E−08 |
| Trip11 | NA | 0.347886673 | 4.15E−08 |
| Slc16a2 | NA | −0.251969763 | 4.57E−08 |
| Ctsc | NA | 0.361021766 | 4.80E−08 |
| Megf9 | NA | 0.276890033 | 6.26E−08 |
| Mrpl28 | NA | −0.269896048 | 6.87E−08 |
| Rpl34 | NA | −0.297598539 | 8.13E−08 |
| Mrpl34 | NA | −0.283750306 | 8.42E−08 |
| Mgea5 | NA | 0.32630901 | 9.07E−08 |
| C1d | NA | −0.255261652 | 9.45E−08 |
| Gcgr | NA | −0.257636913 | 1.04E−07 |
| Sptan1 | NA | 0.312663889 | 1.10E−07 |
| Zfp259 | NA | 0.255636639 | 1.10E−07 |
| Xrn2 | NA | 0.298471923 | 1.28E−07 |
| Hipk1 | NA | 0.291600227 | 1.40E−07 |
| Alcam | NA | 0.323788815 | 1.46E−07 |
| Gnas | NA | 0.258853087 | 1.47E−07 |
| Mapkapk2 | NA | 0.31382654 | 1.63E−07 |
| Coasy | NA | −0.253428655 | 1.78E−07 |
| Gars | NA | 0.257638792 | 2.09E−07 |
| Diap1 | NA | 0.325941798 | 2.10E−07 |
| Rab6a | NA | 0.32343226 | 2.23E−07 |
| Pvrl2 | NA | −0.261260991 | 2.66E−07 |
| 2810428115Rik | NA | −0.263117928 | 2.80E−07 |
| Nucks1 | NA | 0.355785584 | 2.82E−07 |
| Hnrnpu | NA | 0.339415607 | 3.35E−07 |
| Chuk | NA | 0.372437977 | 5.21E−07 |
| Serbp1 | NA | 0.322652522 | 5.43E−07 |
| Ctnnd1 | NA | 0.251848095 | 5.48E−07 |
| Nploc4 | NA | 0.291804954 | 5.69E−07 |
| Zfp106 | NA | 0.336920255 | 6.34E−07 |
| Arhgdia | NA | 0.256326455 | 7.14E−07 |
| BC005537 | NA | 0.354337911 | 1.05E−06 |
| Akr1e1 | NA | −0.258207619 | 1.09E−06 |
| Eific | NA | 0.301979757 | 1.10E−06 |
| Tmem86b | NA | −0.258236533 | 1.18E−06 |
| Mrps12 | NA | −0.250441318 | 1.26E−06 |
| Cdh1 | NA | 0.269696738 | 1.61E−06 |
| Ddx3y | NA | 0.339517654 | 1.83E−06 |
| Ndufa5 | NA | −0.251988986 | 1.96E−06 |
| Ppm1g | NA | 0.252225215 | 2.04E−06 |
| Elovl5 | NA | 0.335236661 | 2.54E−06 |
| Impad1 | NA | 0.32267465 | 3.04E−06 |
| Tab2 | NA | 0.348412001 | 3.51E−06 |
| Cyp3a13 | NA | 0.284411943 | 4.93E−06 |
| Pgls | NA | −0.274714156 | 5.62E−06 |
| Actr3 | NA | 0.275997927 | 5.66E−06 |
| St3gal4 | NA | 0.275033534 | 5.81E−06 |
| 4932438A13Rik | NA | 0.287850722 | 7.97E−06 |
| Abce1 | NA | 0.279955892 | 8.43E−06 |
| Pde3b | NA | −0.258970093 | 8.55E−06 |
| Smoc1 | NA | 0.26157412 | 8.57E−06 |
| 4931406C07Rik | NA | −0.254363633 | 9.62E−06 |
| Rmnd5a | NA | 0.318903459 | 1.09E−05 |
| Ash11 | NA | 0.265693821 | 1.23E−05 |
| Sf3b1 | NA | 0.28884767 | 1.32E−05 |
| Spag9 | NA | 0.318820155 | 1.33E−05 |
| Nek7 | NA | 0.291098417 | 1.63E−05 |
| Eif4a1 | NA | 0.251906299 | 2.25E−05 |
| Usp7 | NA | 0.287320099 | 2.90E−05 |
| Tbca | NA | −0.27261031 | 3.43E−05 |
| Pi4k2a | NA | 0.353561144 | 3.46E−05 |
| Prpf19 | NA | 0.25876049 | 3.47E−05 |
| Usp34 | NA | 0.285942387 | 5.16E−05 |
| Slc16a10 | NA | −0.254826191 | 9.62E−05 |
| Eea1 | NA | 0.261201113 | 0.000132332 |
| Sars | NA | 0.276853119 | 0.00019395 |
| Herc4 | NA | 0.267474882 | 0.000255647 |
| Gadd45a | NA | 0.282867411 | 0.000337547 |
| Bc1af1 | NA | 0.26848767 | 0.000425718 |
| Gm5424 | NA | 0.269800095 | 0.000520866 |
| Slc35d1 | NA | 0.309435259 | 0.000579989 |
| Cu11 | NA | 0.259206487 | 0.000597201 |
| Cu13 | NA | 0.278802183 | 0.000803381 |
| Slc30a1 | NA | 0.250570123 | 0.000818871 |
| Eifia | NA | 0.314713983 | 0.00096785 |
| Tln1 | NA | 0.321033046 | 0.001767724 |
| Zfand5 | NA | 0.28815165 | 0.00189648 |
| Glul | 0.00256055 | 0.379494728 | 0.00256055 |
| Eif4ebp1 | NA | 0.261682735 | 0.003063164 |
| Hectd1 | NA | 0.30802968 | 0.003395612 |
| Gldc | NA | 0.280156601 | 0.00370491 |
| Rassf3 | NA | 0.291236708 | 0.004723315 |
| Nudt4 | NA | 0.25418892 | 0.007016738 |
| Rock1 | NA | 0.2607635 | 0.009308994 |
| Rbm39 | NA | 0.265732671 | 0.012545192 |
| Abcc2 | NA | 0.289681267 | 0.018266512 |
| Naa50 | NA | 0.265659058 | 0.019114384 |
| Pa2g4 | NA | 0.289349263 | 0.02381992 |
| Riok3 | NA | 0.291562038 | 0.055677881 |
| Myo1b | NA | 0.262427327 | 0.075213075 |
| Eifib | NA | 0.250328103 | 0.08550598 |
| Ldlr | NA | 0.282025946 | 0.146582345 |
| Psap | NA | 0.360973931 | 0.257800256 |
| Gda | NA | 0.627476336 | 0.266276129 |
| Gnai2 | NA | 1 | 0.281667665 |

TABLE 8B
Differentially expressed genes between treatment (PH and UT).

| ID | PH3logFC | PH48logFC | PH120logFC | PH3p | PH48p |
|---|---|---|---|---|---|
| Saa2 | 2.971862499 | 4.43818759 | 3.598008691 | 0 | 0 |
| Mt1 | 2.610854994 | 1.733946488 | 1.65972779 | 0 | 9.88E−293 |
| Mt2 | 2.6060431 | 1.558205591 | 1.471840722 | 0 | 2.04E−271 |
| Saa1 | 2.202156726 | 3.650785996 | 2.959370227 | 0 | 0 |
| Steap4 | 1.890015167 | 0.631013696 | 0.931387724 | 0 | 4.71E−63 |
| Serpina3n | 1.759758939 | 1.082720183 | 1.205364333 | 0 | 4.67E−169 |
| Lcn2 | 1.499141183 | 2.39758181 | 2.127649068 | 0 | 0 |
| Lpin1 | 1.327657625 | NA | 0.382955017 | 0 | NA |
| n-R5-8s1 | 1.289598504 | 1.223121237 | 1.066654791 | 0 | 2.94E−283 |
| Igfbp1 | 1.288789902 | 0.466237818 | 0.488899754 | 0 | 2.67E−25 |
| Cxcl1 | 1.230398019 | 0.353012683 | NA | 0 | 7.28E−05 |
| Scd1 | 1.081676999 | NA | 0.787835318 | 0 | NA |
| Gm26924 | 1.063463323 | 1.445355764 | 1.379756734 | 0 | 0 |
| Il1r1 | 0.998168131 | 0.299449962 | 0.63980318 | 0 | 2.79E−18 |
| Fga | 0.981249724 | 0.968748472 | 1.039432587 | 0 | 9.27E−246 |
| Gm24601 | 0.924568273 | 0.675379727 | 0.675086715 | 0 | 2.68E−176 |
| Nnmt | 0.901052904 | 0.768076272 | 0.508818069 | 0 | 3.84E−107 |
| Itih4 | 0.887186241 | 1.171742648 | 1.18011817 | 0 | 0 |
| St3gal5 | 0.882761517 | NA | NA | 0 | NA |
| Tacc2 | 0.877623973 | NA | NA | 0 | NA |
| Lrg1 | 0.870250525 | 1.051046086 | 1.260509735 | 0 | 1.54E−213 |
| Fam134b | 0.865578177 | NA | NA | 0 | NA |
| Tat | 0.857965136 | 0.369618242 | 0.614979826 | 0 | 2.45E−45 |
| Saa4 | 0.854921603 | 0.635837715 | 1.158596154 | 0 | 6.90E−79 |
| Thrsp | 0.836598219 | −0.374383397 | −0.629247506 | 0 | 1.55E−18 |
| Slc39a14 | 0.831584936 | NA | 0.506662289 | 0 | NA |
| Lars2 | 0.816795688 | 0.849538102 | 1.419129056 | 0 | 4.73E−222 |
| Fgg | 0.809322713 | 0.702140119 | 0.686743278 | 0 | 3.42E−167 |
| Slc38a2 | 0.797899442 | NA | 0.333158401 | 0 | NA |
| Serpina3m | 0.779052745 | 0.519751144 | 0.305171041 | 0 | 1.28E−57 |
| Hp | 0.774785279 | 1.584889895 | 1.107075607 | 0 | 0 |
| Fgb | 0.739519125 | 0.677592366 | 0.605783409 | 0 | 1.41E−179 |
| Gm15564 | 0.735157137 | 0.746211054 | 1.485885928 | 0 | 4.32E−146 |
| Serpina10 | 0.733655099 | 0.871044179 | 0.740578005 | 0 | 3.60E−107 |
| Plin2 | 0.720463076 | NA | NA | 0 | NA |
| Fmo5 | 0.690160801 | 0.379271143 | 0.337493236 | 0 | 1.75E−43 |
| Fkbp5 | 0.663304836 | NA | NA | 0 | NA |
| Slc25a47 | 0.635928777 | −0.555532536 | NA | 0 | 2.81E−104 |
| Mat1a | 0.623734998 | 0.378728792 | 0.519634823 | 0 | 5.65E−41 |
| Acox1 | 0.540022158 | NA | 0.292890336 | 0 | NA |
| Aldob | 0.490200848 | −0.254127817 | NA | 0 | 2.25E−64 |
| C3 | 0.482674202 | 0.982337034 | 1.071950932 | 0 | 0 |
| B3galt1 | 0.788734938 | NA | 0.251993915 | 3.52E−301 | NA |
| Fn1 | 0.684266092 | 0.469732274 | 1.018772906 | 5.56E−298 | 5.12E−32 |
| Hnf4a | 0.644195253 | NA | 0.470873585 | 5.68E−290 | NA |
| Btg2 | 0.772686832 | 0.280923811 | 0.386164963 | 7.24E−290 | 2.37E−05 |
| H3f3b | 0.755567107 | NA | NA | 4.44E−288 | NA |
| Aass | 0.705615345 | NA | 0.312355615 | 1.07E−286 | NA |
| Sephs2 | 0.618707003 | NA | 0.604852301 | 1.06E−281 | NA |
| Cldn14 | 0.511583526 | NA | NA | 4.32E−281 | NA |
| Rcan1 | 0.509239866 | NA | NA | 4.58E−279 | NA |
| Hspb8 | 0.667847253 | NA | 0.52383406 | 9.19E−267 | NA |
| Arhgef26 | 0.568544702 | NA | 0.276634335 | 4.92E−265 | NA |
| Slc7a2 | 0.622326529 | NA | 0.374380964 | 6.74E−265 | NA |
| Pfkfb3 | 0.426580733 | NA | NA | 3.82E−264 | NA |
| Pck1 | 0.43447474 | NA | 0.495042924 | 4.86E−264 | NA |
| Errfi1 | 0.500850526 | NA | NA | 7.43E−262 | NA |
| Gjb2 | 0.639507436 | 0.328679811 | 0.402811227 | 9.92E−255 | 2.44E−12 |
| Snx10 | 0.558244633 | 0.253822304 | NA | 1.59E−251 | 1.28E−13 |
| Bcl3 | 0.419650143 | NA | NA | 3.05E−251 | NA |
| Hpx | 0.460272423 | 1.443685235 | 0.994363825 | 2.87E−247 | 0 |
| Bach1 | 0.533237374 | NA | NA | 5.54E−247 | NA |
| Tifa | 0.392339477 | NA | 0.321485305 | 3.72E−241 | NA |
| 2010003K11Rik | 0.372219971 | NA | NA | 1.06E−239 | NA |
| Pzp | 0.387351113 | 0.382796555 | 0.636851805 | 2.85E−237 | 2.61E−52 |
| Tiparp | 0.489122153 | NA | NA | 7.07E−236 | NA |
| Rapgef4 | 0.588317735 | NA | NA | 4.64E−233 | NA |
| Mup10 | 0.428268029 | 0.281431551 | 0.254194035 | 1.14E−231 | 4.82E−45 |
| Egfr | 0.555834247 | NA | 0.65440823 | 5.47E−228 | NA |
| mmu-mir-6236 | 0.468866739 | 0.632369146 | 1.257248389 | 4.51E−221 | 5.49E−97 |
| Etnk2 | 0.592353201 | NA | NA | 7.87E−221 | NA |
| Mfsd2a | 0.496351884 | NA | NA | 1.64E−219 | NA |
| Txnip | 0.552612237 | −0.310098222 | NA | 1.04E−218 | 1.24E−19 |
| Arg1 | 0.381616351 | NA | NA | 2.23E−218 | NA |
| Gpt2 | 0.533331785 | NA | NA | 3.67E−218 | NA |
| Slc25a25 | 0.544465891 | NA | 0.442662636 | 2.55E−217 | NA |
| Actb | 0.456981443 | 0.399035393 | NA | 2.15E−210 | 2.62E−44 |
| Xbp1 | 0.539986 | NA | NA | 1.34E−208 | NA |
| Upp2 | 0.541753512 | −0.265836776 | NA | 2.02E−206 | 1.03E−24 |
| Fbf1 | 0.298573909 | NA | NA | 4.53E−206 | NA |
| Plin5 | 0.489257529 | NA | NA | 1.01E−205 | NA |
| Plk3 | 0.488016367 | NA | NA | 8.22E−205 | NA |
| Trib1 | 0.581754281 | NA | NA | 1.57E−204 | NA |
| Mtss1 | 0.552137981 | NA | 0.339863924 | 2.07E−204 | NA |
| Klf6 | 0.3616937 | NA | NA | 8.00E−203 | NA |
| Itih3 | 0.469631651 | 1.223022315 | 1.177912468 | 1.12E−197 | 4.04E−297 |
| Slc10a2 | 0.30563824 | NA | NA | 6.54E−197 | NA |
| Sdc4 | 0.43560504 | NA | 0.315436107 | 2.70E−193 | NA |
| Atf3 | 0.597274889 | NA | 0.262281273 | 1.23E−191 | NA |
| Ptp4a1 | 0.393137367 | NA | NA | 6.10E−190 | NA |
| Prg4 | 0.480504574 | 0.3555459X5 | 0.810835665 | 4.35E−189 | 1.45E−24 |
| Rgs16 | 0.438105488 | NA | NA | 3.55E−188 | NA |
| Gpcpd1 | 0.53780646 | NA | NA | 5.36E−188 | NA |
| Rhob | 0.37207281 | NA | NA | 4.39E−187 | NA |
| Glul | 0.478908893 | 0.611936929 | 0.495360357 | 1.01E−186 | 1 |
| Fam35a | 0.470461753 | NA | NA | 7.67E−186 | NA |
| Klf10 | 0.304779554 | NA | NA | 1.06E−185 | NA |
| Egr1 | 0.620480469 | NA | 0.271667356 | 7.27E−185 | NA |
| Apcs | 0.573439593 | 2.049485412 | 0.965147497 | 2.06E−182 | 0 |
| Shb | 0.430613558 | NA | NA | 4.03E−182 | NA |
| Orm1 | 0.463411533 | 1.823760769 | 1.05827481 | 4.58E−180 | 0 |
| Fgl1 | 0.49338252 | 1.561880768 | 1.379926505 | 2.36E−175 | 0 |
| 1810055G02Rik | 0.382584093 | NA | NA | 7.73E−175 | NA |
| Stat3 | 0.460515229 | 0.308012615 | 0.483904634 | 3.58E−174 | 2.82E−05 |
| Abca1 | 0.50506234 | NA | 0.370856836 | 3.19E−172 | NA |
| Igfbp4 | 0.387861679 | NA | 0.411174683 | 6.69E−171 | NA |
| Got1 | 0.542891511 | NA | 0.331700716 | 7.82E−171 | NA |
| Cdkn1a | 0.449325471 | NA | NA | 7.06E−169 | NA |
| Apob | 0.416824707 | NA | 0.505397578 | 8.05E−169 | NA |
| Gfra1 | 0.474694426 | NA | NA | 4.73E−168 | NA |
| Ivns1abp | 0.46254094 | NA | NA | 9.44E−168 | NA |
| Slc41a2 | 0.3685439 | 0.318516467 | 0.441321771 | 2.22E−166 | 5.60E−25 |
| Slc25a51 | 0.372157664 | NA | NA | 1.11E−164 | NA |
| Myo1e | 0.399456672 | NA | NA | 7.83E−162 | NA |
| Gm23935 | 0.498066925 | 0.562414557 | 1.207244314 | 8.57E−162 | 2.21E−73 |
| Bhlhe40 | 0.403249298 | NA | 0.370112004 | 3.04E−158 | NA |
| Ddx3x | 0.47100162 | NA | NA | 1.36E−155 | NA |
| Tmem87b | 0.288330587 | NA | NA | 8.72E−155 | NA |
| Dak | 0.481947664 | NA | NA | 1.71E−153 | NA |
| Slc25a22 | 0.390931532 | NA | NA | 7.19E−153 | NA |
| Fasn | 0.467168076 | NA | NA | 2.55E−152 | NA |
| Fh1 | 0.471428771 | NA | NA | 7.97E−152 | NA |
| Rnf103 | 0.438540394 | NA | NA | 1.39E−149 | NA |
| Sds | 0.787094904 | −0.352238286 | NA | 5.50E−149 | 7.99E−43 |
| B4galt1 | 0.321380868 | NA | NA | 4.77E−146 | NA |
| Cps1 | 0.336429448 | NA | 0.312297912 | 1.19E−145 | NA |
| Atp1b1 | 0.424801231 | NA | NA | 1.32E−145 | NA |
| Jmjd1c | 0.39612601 | NA | NA | 1.05E−144 | NA |
| Gm26917 | 0.46739621 | 0.475554164 | 0.443747552 | 2.42E−144 | 2.52E−05 |
| Junb | 0.458500682 | NA | NA | 1.57E−143 | NA |
| 4933426M11Rik | 0.28624304 | NA | NA | 1.78E−142 | NA |
| Pik3r1 | 0.389193614 | NA | NA | 1.89E−142 | NA |
| D10Wsu102e | 0.256022156 | NA | NA | 1.03E−141 | NA |
| Hyou1 | 0.431109873 | 0.315760783 | 0.496156079 | 3.60E−141 | 0.000165574 |
| Glud1 | 0.341724783 | 0.359099708 | 0.307823999 | 1.11E−139 | 4.69E−47 |
| Cpt1a | 0.42860734 | NA | 0.276570679 | 3.31E−139 | NA |
| Epas1 | 0.38425941 | NA | NA | 2.12E−138 | NA |
| Jun | 0.479246114 | NA | NA | 2.64E−135 | NA |
| Mbd1 | 0.348094524 | NA | NA | 2.44E−134 | NA |
| Rnd3 | 0.285147682 | NA | NA | 4.66E−134 | NA |
| Litaf | 0.396007707 | NA | NA | 2.34E−132 | NA |
| Ptpn1 | 0.268263225 | NA | NA | 6.92E−130 | NA |
| Bcl2l1 | 0.280315334 | NA | NA | 1.17E−128 | NA |
| Grn | 0.403315761 | 0.293077345 | 0.316049742 | 3.44E−128 | 4.47E−10 |
| Tns1 | 0.33981795 | NA | NA | 4.58E−128 | NA |
| Slc25a15 | 0.402049896 | NA | 0.393990477 | 6.99E−128 | NA |
| Gadd45g | 0.392031882 | −0.336240737 | NA | 7.78E−128 | 3.29E−30 |
| Wbp1l | 0.409138156 | NA | NA | 1.29E−127 | NA |
| Foxa3 | 0.307992015 | NA | NA | 2.28E−126 | NA |
| Myh9 | 0.399128031 | 0.438269269 | 0.631035657 | 3.04E−126 | 1.59E−15 |
| Fos | 0.524733327 | NA | NA | 3.55E−125 | NA |
| Id2 | 0.430147785 | −0.333366521 | NA | 5.03E−124 | 1.37E−46 |
| Itih2 | 0.336714211 | 0.312933325 | NA | 1.56E−122 | 2.29E−24 |
| Plg | 0.26619661 | NA | 0.596524395 | 1.35E−121 | NA |
| Qsox1 | 0.378486857 | 0.551776463 | 0.725062115 | 3.75E−121 | 2.24E−55 |
| Ccnl1 | 0.341338448 | NA | NA | 1.67E−120 | NA |
| Eef1a1 | 0.340768186 | 0.254969818 | NA | 6.14E−119 | 1.25E−12 |
| Ier2 | 0.452598264 | NA | NA | 1.60E−118 | NA |
| Elovl6 | 0.37962641 | NA | NA | 5.92E−117 | NA |
| Nedd4l | 0.271827098 | NA | NA | 1.51E−116 | NA |
| Ppip5k2 | 0.30299148 | NA | NA | 3.63E−115 | NA |
| Epb4.1 | 0.349430673 | NA | NA | 1.93E−113 | NA |
| Cfh | 0.312615767 | 0.588715272 | 0.83288399 | 3.46E−113 | 2.52E−103 |
| Ulk1 | 0.297841785 | NA | NA | 5.13E−113 | NA |
| Ell2 | 0.371745977 | NA | NA | 9.30E−113 | NA |
| Tnfrsf1a | 0.346845029 | NA | 0.267537895 | 3.42E−112 | NA |
| Eif4g2 | 0.384302798 | NA | 0.287105046 | 2.51E−110 | NA |
| Ly6e | 0.42022985 | 0.525599776 | 1.093957206 | 1.47E−108 | 5.02E−20 |
| Il6ra | 0.251696836 | NA | NA | 2.93E−108 | NA |
| Lpgat1 | 0.390146943 | 0.347767347 | 0.400787984 | 1.25E−107 | 1.05E−09 |
| Fads2 | 0.323744791 | NA | NA | 4.78E−106 | NA |
| Atl2 | 0.321873809 | NA | NA | 5.02E−106 | NA |
| Dst | 0.329905553 | NA | NA | 7.40E−105 | NA |
| Jak1 | 0.362766687 | NA | NA | 2.44E−103 | NA |
| Nfil3 | 0.338730712 | NA | NA | 5.23E−103 | NA |
| Elovl5 | 0.382267188 | NA | NA | 1.08E−102 | NA |
| Mgll | 0.321840174 | NA | NA | 6.61E−101 | NA |
| Sult1a1 | 0.371023537 | 0.344836675 | NA | 2.67E−99 | 2.16E−12 |
| Man2a1 | 0.369999358 | NA | 0.323273082 | 3.60E−98 | NA |
| Eef2 | 0.289562847 | NA | 0.321705764 | 1.70E−97 | NA |
| Aff4 | 0.344186222 | NA | NA | 7.00E−97 | NA |
| Palmd | 0.284673594 | NA | NA | 9.43E−97 | NA |
| Zfp36 | 0.407014847 | NA | NA | 1.05E−96 | NA |
| Eif1a | 0.295219945 | NA | NA | 7.80E−96 | NA |
| Slc3a1 | 0.290117272 | 0.321802388 | 0.381791335 | 1.93E−94 | 1.78E−11 |
| Ifitm3 | 0.331673739 | NA | 0.506857449 | 3.64E−94 | NA |
| Atp2a2 | 0.350363266 | NA | 0.263222967 | 8.21E−94 | NA |
| Nop58 | 0.257566653 | NA | NA | 1.12E−93 | NA |
| Enpp2 | 0.316882991 | NA | NA | 4.48E−93 | NA |
| Gm2a | 0.341145926 | NA | NA | 6.61E−93 | NA |
| Cyb5r3 | 0.337684684 | NA | NA | 1.90E−92 | NA |
| Apoa4 | 0.609214916 | 1.186863065 | NA | 2.34E−92 | 3.01E−137 |
| Aldh1l1 | 0.277372649 | NA | 0.564932962 | 2.62E−88 | NA |
| Eci2 | 0.288159513 | NA | NA | 5.23E−88 | NA |
| Slc38a3 | 0.292400046 | NA | 0.353500212 | 1.28E−87 | NA |
| Dusp1 | 0.300628481 | NA | NA | 1.26E−86 | NA |
| Igf1 | 0.277138761 | NA | NA | 2.86E−86 | NA |
| Serinc3 | 0.293964974 | NA | NA | 2.71E−85 | NA |
| Tgm2 | 0.316575272 | 0.37123494 | 0.342526865 | 5.50E−85 | 5.94E−16 |
| C8a | 0.294064237 | NA | 0.404198047 | 1.39E−84 | NA |
| Txnrd1 | 0.314979852 | 0.252234489 | NA | 3.22E−84 | 0.032529357 |
| Asl | 0.297673772 | NA | NA | 1.76E−83 | NA |
| Por | 0.290745567 | NA | NA | 3.93E−83 | NA |
| Sec14l4 | 0.285169227 | NA | NA | 5.54E−83 | NA |
| Uroc1 | 0.329587334 | NA | 0.459322934 | 1.13E−82 | NA |
| Lrp6 | 0.270740438 | NA | NA | 1.18E−82 | NA |
| Atf6 | 0.280521162 | NA | NA | 1.55E−82 | NA |
| Ddi2 | 0.302519699 | NA | NA | 2.71E−82 | NA |
| Dstn | 0.271381717 | NA | NA | 2.90E−82 | NA |
| Id3 | 0.399520373 | NA | NA | 9.94E−82 | NA |
| H6pd | 0.300492669 | NA | 0.491965789 | 1.91E−81 | NA |
| Xdh | 0.27224472 | NA | 0.373181784 | 8.54E−81 | NA |
| Tmed5 | 0.296018777 | NA | NA | 1.36E−80 | NA |
| G6pc | 0.341403933 | −0.259264856 | NA | 2.15E−80 | 3.71E−17 |
| Mttp | 0.296651415 | NA | NA | 2.14E−79 | NA |
| Angptl4 | 0.315315425 | NA | NA | 2.44E−79 | NA |
| Nrp1 | 0.268796793 | NA | NA | 7.49E−79 | NA |
| Fgf1 | 0.287983029 | NA | NA | 7.97E−79 | NA |
| Prox1 | 0.31520499 | NA | NA | 3.03E−77 | NA |
| Gck | 0.260705425 | NA | NA | 6.82E−77 | NA |
| Picalm | 0.276902165 | NA | NA | 1.27E−76 | NA |
| Lbp | 0.264248666 | NA | 0.656662588 | 4.58E−76 | NA |
| Azin1 | 0.268891169 | NA | NA | 7.99E−76 | NA |
| Ddx5 | 0.251675045 | NA | NA | 3.02E−75 | NA |
| Cpb2 | 0.280302458 | 0.395699062 | 0.403790403 | 1.59E−74 | 1.09E−38 |
| Il6st | 0.262348153 | NA | 0.382881893 | 3.02E−73 | NA |
| Rmnd5a | 0.273473023 | NA | NA | 5.48E−73 | NA |
| Trp53inp1 | 0.269278401 | NA | NA | 1.01E−72 | NA |
| Ctsb | 0.268200419 | 0.310468937 | 0.553974491 | 1.09E−72 | 2.17E−12 |
| Acsl4 | 0.250793464 | 0.273275871 | NA | 5.43E−70 | 0.003086449 |
| Elovl2 | 0.285038606 | NA | NA | 2.60E−69 | NA |
| Iqgap2 | 0.283192936 | NA | NA | 5.91E−69 | NA |
| Lrp1 | 0.310226628 | 0.314138344 | 0.914046975 | 2.25E−68 | 2.75E−05 |
| Arhgef12 | 0.280147378 | NA | 0.304865834 | 1.49E−67 | NA |
| Pim3 | 0.280137371 | −0.255337561 | NA | 3.27E−67 | 2.68E−22 |
| C4bp | 0.26668764 | 0.530516995 | 0.505363658 | 4.30E−67 | 7.45E−90 |
| Lrpprc | 0.257016288 | NA | NA | 6.41E−67 | NA |
| Atp1a1 | 0.261718912 | NA | NA | 2.96E−65 | NA |
| Copa | 0.251410027 | NA | 0.294256247 | 1.69E−64 | NA |
| Acly | 0.287663867 | NA | NA | 4.67E−64 | NA |
| Rcl1 | 0.273905375 | NA | NA | 1.37E−63 | NA |
| Alas1 | 0.283195713 | 0.423999349 | 0.300367757 | 6.82E−63 | 7.65E−32 |
| Kmo | 0.262269388 | NA | 0.305370487 | 2.45E−62 | NA |
| Ppp1r3b | 0.259418401 | NA | NA | 1.58E−61 | NA |
| Zfp36l1 | 0.271823824 | NA | NA | 4.54E−61 | NA |
| Efna1 | 0.256152989 | NA | 0.268455179 | 2.55E−60 | NA |
| Ctsl | 0.256520866 | NA | NA | 1.56E−59 | NA |
| Ptplad1 | 0.255273084 | NA | NA | 2.04E−59 | NA |
| Hdlbp | 0.267317937 | NA | 0.394030651 | 3.33E−59 | NA |
| Pnpla7 | 0.251226201 | NA | NA | 4.93E−58 | NA |
| Fads1 | 0.267264464 | NA | NA | 2.35E−57 | NA |
| Nedd4 | 0.259978699 | NA | NA | 7.35E−56 | NA |
| Mtus1 | 0.268076242 | NA | 0.385483144 | 2.92E−54 | NA |
| Cyp2c70 | 0.25600888 | NA | 0.511802822 | 8.20E−51 | NA |
| Brd2 | 0.258446853 | NA | NA | 8.84E−51 | NA |
| Saa3 | 0.298054041 | 2.199786317 | NA | 7.93E−23 | 0 |
| Orm2 | NA | 2.405086745 | 1.536747401 | NA | 0 |
| Ambp | NA | 0.4774812 | 0.496213815 | NA | 2.69E−163 |
| Trf | NA | 0.436833817 | NA | NA | 1.60E−148 |
| Rnase4 | NA | 0.836959148 | 1.031156304 | NA | 4.28E−141 |
| Prtn3 | NA | 0.327267898 | 0.350182853 | NA | 8.65E−141 |
| Fam25c | NA | 0.96762143 | NA | NA | 2.76E−118 |
| Cp | NA | 0.620221202 | 0.655511152 | NA | 1.41E−115 |
| Dhcr24 | NA | 0.759730578 | 0.551421264 | NA | 1.65E−115 |
| Kng1 | NA | 0.419944891 | 0.546835494 | NA | 3.92E−102 |
| Csf2ra | NA | 0.342712543 | NA | NA | 3.48E−100 |
| D17H6S56E-5 | NA | 0.560241333 | NA | NA | 2.13E−95 |
| Cxcl14 | NA | 0.268462966 | NA | NA | 7.87E−89 |
| Hsp90ab1 | NA | 0.455851506 | 0.323037124 | NA | 1.23E−83 |
| Tmem176a | NA | 0.675060004 | 0.67082422 | NA | 3.59E−81 |
| Isyna1 | NA | 0.320415943 | 0.270110032 | NA | 8.49E−80 |
| Cd5l | NA | 0.460825248 | 0.49371428 | NA | 2.07E−79 |
| Ccnd1 | NA | 0.385849227 | NA | NA | 6.27E−78 |
| P4hb | NA | 0.38506968 | 0.369961971 | NA | 1.73E−74 |
| Tmem176b | NA | 0.612457008 | 0.713749974 | NA | 4.03E−74 |
| Gas6 | NA | 0.35878079 | 0.269480866 | NA | 5.31E−74 |
| Hmgcs1 | −0.258009688 | 0.702526059 | −0.387891871 | 1.33E−13 | 4.05E−72 |
| Ang | 0.315315425 | 0.644516227 | 0.599634512 | 2.44E−79 | 2.84E−71 |
| Marco | NA | 0.30539135 | NA | NA | 1.17E−70 |
| Ahsg | NA | 0.281450293 | 0.32482682 | NA | 7.29E−69 |
| Gm12744 | NA | 0.350643059 | NA | NA | 1.69E−65 |
| Clu | NA | 0.374886754 | 0.745288027 | NA | 3.60E−64 |
| Rbp1 | NA | 0.396801127 | 0.32795786 | NA | 5.19E−64 |
| C1qb | NA | 0.361309767 | 0.337494489 | NA | 2.50E−59 |
| Mki67 | NA | 0.39305852 | NA | NA | 1.59E−57 |
| Acnat2 | NA | 0.33547101 | NA | NA | 1.88E−56 |
| Top2a | NA | 0.33486876 | NA | NA | 1.10E−54 |
| Clec4f | NA | 0.380337956 | 0.487336922 | NA | 1.18E−53 |
| Rpl41 | NA | 0.444557582 | NA | NA | 1.56E−51 |
| Cdca3 | NA | 0.286393603 | NA | NA | 1.87E−51 |
| Ifitm2 | NA | 0.526891524 | 0.281474354 | NA | 1.07E−50 |
| Dbi | NA | 0.360631658 | NA | NA | 9.49E−48 |
| 1100001G20Rik | NA | 0.310291029 | NA | NA | 4.13E−43 |
| Cdc20 | NA | 0.272554242 | NA | NA | 3.12E−38 |
| Cd302 | NA | 0.384976006 | NA | NA | 1.82E−37 |
| Csf1r | NA | 0.284684372 | NA | NA | 3.79E−37 |
| Gstm2 | NA | 0.466218536 | NA | NA | 3.99E−35 |
| Apoc2 | NA | 0.517142972 | NA | NA | 6.48E−35 |
| Krt18 | NA | 0.520354076 | NA | NA | 5.88E−34 |
| Tmsb4x | NA | 0.347657914 | 0.341035762 | NA | 7.53E−31 |
| Dnajc12 | NA | 0.312907586 | NA | NA | 2.54E−30 |
| Lyz2 | NA | 0.275390862 | 0.402735723 | NA | 1.21E−28 |
| BC048546 | NA | 0.287060939 | NA | NA | 1.16E−27 |
| Gstm3 | −0.470077922 | 0.793361403 | NA | 8.30E−17 | 1.46E−26 |
| Hspa8 | NA | 0.302152434 | NA | NA | 4.31E−21 |
| Dynll1 | NA | 0.289016997 | NA | NA | 1.79E−19 |
| Tspan4 | NA | 0.32084381 | NA | NA | 3.41E−19 |
| Kng2 | NA | 0.3169695 | 0.368809413 | NA | 2.95E−18 |
| 2200002D01Rik | NA | 0.257614019 | NA | NA | 1.71E−17 |
| Cyp51 | NA | 0.40339747 | −0.324000937 | NA | 4.34E−17 |
| Cyp7a1 | NA | 0.598824806 | NA | NA | 5.91E−17 |
| Mlec | NA | 0.400321297 | NA | NA | 1.46E−15 |
| Smc4 | NA | 0.338338192 | NA | NA | 3.80E−14 |
| Hrg | NA | 0.270451116 | 0.58732025 | NA | 6.86E−14 |
| Sdc1 | NA | 0.330139692 | NA | NA | 1.43E−12 |
| Hspa5 | NA | 0.270882455 | 0.339769129 | NA | 2.27E−12 |
| Dap | NA | 0.290008119 | 0.465257779 | NA | 3.97E−12 |
| Hsph1 | NA | 0.345997935 | 0.425977247 | NA | 1.02E−10 |
| Mrap | NA | 0.280974611 | NA | NA | 1.04E−10 |
| Creld2 | NA | 0.362477611 | 0.307177214 | NA | 1.11E−08 |
| Cpox | NA | 0.294319953 | NA | NA | 2.98E−08 |
| Ebp | NA | 0.252819864 | NA | NA | 3.69E−07 |
| Fdps | NA | 0.303232732 | −0.254137438 | NA | 3.91E−06 |
| Ugdh | NA | 0.260443532 | NA | NA | 4.68E−05 |
| Tmpo | NA | 0.2573532 | NA | NA | 5.30E−05 |
| Lss | NA | 0.294433908 | NA | NA | 0.000130068 |
| Akr1d1 | NA | 0.258293189 | NA | NA | 0.00030931 |
| Snord13 | NA | 0.408788625 | NA | NA | 0.000327047 |
| Lsr | NA | 0.262580416 | NA | NA | 0.005770468 |
| Tuba1c | NA | 0.302939755 | NA | NA | 0.006056912 |
| Caprin1 | NA | 0.303020851 | 0.295892842 | NA | 0.016747307 |
| Atl3 | NA | 0.255322039 | NA | NA | 0.021123411 |
| Manf | NA | 0.282950631 | NA | NA | 0.023268676 |
| Avpi1 | NA | 0.30186919 | NA | NA | 0.101466193 |
| Krt8 | NA | 0.252276759 | NA | NA | 0.182829207 |
| Sqle | NA | 0.27714068 | NA | NA | 1 |
| Nucks1 | NA | 0.271055696 | NA | NA | 1 |
| Lect2 | −0.274252442 | 0.288512824 | NA | 1.07E−19 | 1 |
| Oat | −0.255537891 | 0.741679089 | NA | 4.82E−23 | 1 |
| Tubb5 | NA | 0.313824522 | NA | NA | 1 |
| Tubb4b | NA | 0.28149027 | NA | NA | 1 |
| C4b | 0.26668764 | 0.530516995 | 0.820022988 | 4.30E−67 | 7.45E−90 |
| Vtn | NA | NA | 0.516935534 | NA | NA |
| Hc | NA | NA | 0.611232959 | NA | NA |
| Orm3 | NA | NA | 0.517250103 | NA | NA |
| Serping1 | NA | NA | 0.535830748 | NA | NA |
| Mpeg1 | NA | NA | 0.541170007 | NA | NA |
| Cml2 | NA | NA | 0.486040271 | NA | NA |
| Itih1 | NA | NA | 0.496617676 | NA | NA |
| Comt | NA | NA | 0.384473508 | NA | NA |
| Calr | NA | NA | 0.391955529 | NA | NA |
| Scara5 | NA | NA | 0.285535495 | NA | NA |
| Cfi | NA | NA | 0.390834066 | NA | NA |
| Mug2 | NA | NA | 0.536577024 | NA | NA |
| H2-D1 | NA | NA | 0.433183706 | NA | NA |
| Lpl | NA | NA | 0.355011922 | NA | NA |
| Col18a1 | NA | NA | 0.459095159 | NA | NA |
| Wfdc17 | NA | NA | 0.317329894 | NA | NA |
| Fetub | NA | NA | 0.425407474 | NA | NA |
| Serpinf2 | NA | NA | 0.361403772 | NA | NA |
| H2-Q10 | NA | NA | 0.250632773 | NA | NA |
| Aldh2 | NA | NA | 0.296492218 | NA | NA |
| Cyp3a13 | NA | NA | 0.420680082 | NA | NA |
| F5 | NA | NA | 0.436709177 | NA | NA |
| Ldhd | NA | NA | 0.387086477 | NA | NA |
| Pcx | NA | NA | 0.367136085 | NA | NA |
| Fah | NA | NA | 0.325518495 | NA | NA |
| Abhd2 | NA | NA | 0.267241512 | NA | NA |
| Ugt1a1 | NA | NA | 0.370092558 | NA | NA |
| Cyr61 | NA | NA | 0.302881921 | NA | NA |
| Pros1 | NA | NA | 0.26812884 | NA | NA |
| Slc22a7 | NA | NA | 0.307857498 | NA | NA |
| Ctss | NA | NA | 0.270183132 | NA | NA |
| P1xnb2 | NA | NA | 0.381208682 | NA | NA |
| Gne | NA | NA | 0.355262513 | NA | NA |
| Abcb11 | NA | NA | 0.351857633 | NA | NA |
| Cpn2 | NA | NA | 0.349996687 | NA | NA |
| Lgals9 | NA | NA | 0.327550674 | NA | NA |
| Hamp2 | NA | NA | 0.337201585 | NA | NA |
| Psap | NA | NA | 0.318472244 | NA | NA |
| Mafb | NA | NA | 0.356246663 | NA | NA |
| Cd36 | NA | NA | 0.291074527 | NA | NA |
| Pdia3 | NA | NA | 0.276156858 | NA | NA |
| Habp2 | NA | NA | 0.313462443 | NA | NA |
| Apon | NA | NA | 0.318989204 | NA | NA |
| Nbr1 | NA | NA | 0.337157409 | NA | NA |
| Slc25a23 | NA | NA | 0.332651576 | NA | NA |
| F9 | NA | NA | 0.303980935 | NA | NA |
| Iigp1 | NA | NA | 0.305889089 | NA | NA |
| Agt | NA | NA | 0.258205872 | NA | NA |
| Gns | NA | NA | 0.323226146 | NA | NA |
| Bsg | NA | NA | 0.273171859 | NA | NA |
| Scarb1 | NA | NA | 0.32724782 | NA | NA |
| Sardh | NA | NA | 0.28626336 | NA | NA |
| Edem1 | NA | NA | 0.301212478 | NA | NA |
| Abat | NA | NA | 0.305043176 | NA | NA |
| Dpyd | NA | NA | 0.315635393 | NA | NA |
| Myo18a | NA | NA | 0.253700048 | NA | NA |
| St6gal1 | NA | NA | 0.295334926 | NA | NA |
| Tapbp | NA | NA | 0.265071778 | NA | NA |
| Ifih1 | NA | NA | 0.264296168 | NA | NA |
| BC025446 | NA | NA | 0.266911384 | NA | NA |
| Pcdh1 | NA | NA | 0.265458741 | NA | NA |
| 1300002K09Rik | NA | NA | 0.30148927 | NA | NA |
| Plec | NA | NA | 0.267122123 | NA | NA |
| Uba1 | NA | NA | 0.281371963 | NA | NA |
| Kynu | NA | NA | 0.28095477 | NA | NA |
| Rxra | NA | NA | 0.264560061 | NA | NA |
| Tfr2 | NA | NA | 0.268628506 | NA | NA |
| Ctnnd1 | NA | NA | 0.252846161 | NA | NA |
| Fndc3a | NA | NA | 0.273177056 | NA | NA |
| Adh4 | NA | NA | 0.282357284 | NA | NA |
| C130074G19Rik | NA | NA | 0.255729195 | NA | NA |
| Ptprf | NA | NA | 0.27768605 | NA | NA |
| Osbp19 | NA | NA | 0.270713808 | NA | NA |
| Vwa8 | NA | NA | 0.262040545 | NA | NA |
| Plcxd2 | NA | NA | 0.266285463 | NA | NA |
| Serpina6 | NA | NA | 0.269623074 | NA | NA |
| Mbl2 | NA | −0.254854549 | 0.268002193 | NA | 5.49E−35 |
| Pid1 | NA | NA | 0.263310286 | NA | NA |
| Cmah | NA | NA | 0.251319774 | NA | NA |
| Aldh1b1 | NA | NA | 0.360480753 | NA | NA |
| Rgn | NA | NA | 0.261094268 | NA | NA |
| Ifi27 | NA | NA | 0.274586258 | NA | NA |
| Gulo | NA | NA | 0.282891961 | NA | NA |
| Slc1a2 | NA | NA | 0.262926395 | NA | NA |
| Mup3 | −0.526410044 | −1.423759103 | −1.310718039 | 0 | 0 |
| Cyb5 | −0.71965743 | −0.486476838 | −0.751689441 | 0 | 1.24E−108 |
| mt-Rnr1 | −0.872186844 | NA | −0.844781496 | 0 | NA |
| mt-Rnr2 | −0.873557854 | −0.51967923 | −0.967453486 | 0 | 2.69E−141 |
| Mup11 | −0.976755523 | −1.415092329 | −1.769206667 | 0 | 0 |
| mt-Nd2 | −1.128776891 | −1.189410874 | −1.350784061 | 0 | 0 |
| mt-Nd1 | −1.136736863 | −1.154630438 | −1.543996447 | 0 | 0 |
| mt-Nd5 | −1.145550707 | −1.397897502 | −1.49645814 | 0 | 1.28E−300 |
| Gm13775 | −1.255657303 | −1.145210977 | −1.171528238 | 0 | 3.97E−216 |
| mt-Cytb | −1.296043506 | −1.378313317 | −1.554026854 | 0 | 0 |
| mt-Co2 | −1.341686736 | −1.417328219 | −1.39473643 | 0 | 5.51E−209 |
| mt-Co1 | −1.381307687 | −1.399110403 | −1.569950123 | 0 | 0 |
| Mup5 | −1.484560547 | −1.768987649 | −1.774609558 | 0 | 2.08E−280 |
| mt-Nd4 | −1.630375247 | −1.673140231 | −1.953205815 | 0 | 0 |
| Mup4 | −1.805301681 | −1.945646142 | −1.909428244 | 0 | 0 |
| Mup6 | −2.386597202 | −2.645373468 | −2.588052175 | 0 | 0 |
| Mup9 | −2.453596744 | −2.219144293 | −2.520658851 | 0 | 0 |
| Mup17 | −2.514472885 | −2.2696211 | −2.382123881 | 0 | 0 |
| Mup18 | −2.617739049 | −2.373348742 | −2.585635085 | 0 | 0 |
| Mup19 | −2.932276479 | −3.632170505 | −3.348975652 | 0 | 0 |
| mt-Co3 | −1.233961828 | −1.212388388 | −1.229639242 | 1.47E−287 | 9.73E−175 |
| Cyp2c29 | −1.027267705 | NA | −0.997502622 | 2.76E−235 | NA |
| Mup16 | −0.848726713 | −0.87654486 | −1.245137265 | 1.76E−222 | 2.12E−149 |
| Cyp2b10 | −1.254972098 | −0.995219016 | −1.247317124 | 1.74E−217 | 1.18E−87 |
| Ankrd55 | −1.464696038 | −1.950942364 | −1.470505577 | 6.28E−208 | 2.52E−247 |
| Ugt2b5 | −0.423085187 | −0.683262994 | −0.716336886 | 8.09E−193 | 1.47E−190 |
| Apoe | −0.26209828 | −0.615882949 | NA | 3.58E−191 | 0 |
| Gsta3 | −0.53784222 | −0.579823037 | −0.796926805 | 2.50E−189 | 2.50E−118 |
| Cyp2c50 | −0.763912176 | −0.467298435 | −0.516529985 | 8.52E−186 | 1.54E−52 |
| Mup21 | −0.668770594 | −1.346645152 | −1.637812283 | 6.47E−176 | 1.32E−290 |
| Cyp2c54 | −0.712859584 | −0.542960224 | −0.632023168 | 1.75E−163 | 2.37E−77 |
| Akr1c6 | −0.43492211 | −0.501143524 | −0.457641734 | 6.85E−150 | 1.04E−103 |
| Apoc4 | −0.356086407 | NA | −0.501759591 | 2.38E−146 | NA |
| mt-Atp6 | −0.852555757 | −0.914635453 | −0.875890108 | 7.98E−137 | 3.35E−117 |
| Mup15 | −1.074409915 | −1.364182598 | −1.359329494 | 5.07E−136 | 2.34E−202 |
| Cyp3a11 | −0.251219977 | NA | −0.803553063 | 8.18E−135 | NA |
| Mgst1 | −0.322742924 | −0.286240934 | −0.36875402 | 2.50E−131 | 3.71E−58 |
| Cyp3a25 | −0.41051207 | −0.34306526 | −0.416456818 | 5.44E−129 | 2.32E−54 |
| Ces1d | −0.385155368 | −0.445283361 | −1.201503329 | 2.67E−121 | 2.43E−81 |
| Mup13 | −0.755608269 | −0.870972392 | −0.875079323 | 4.35E−116 | 1.89E−127 |
| Cyp2c37 | −0.662335734 | −0.412938667 | −0.820386118 | 4.16E−114 | 1.60E−40 |
| Cyp2a5 | −0.938183249 | −0.525680085 | −0.869524548 | 8.06E−111 | 1.48E−38 |
| Serpina3c | −0.554657351 | −0.589926828 | −0.595568461 | 8.44E−109 | 8.61E−89 |
| Adh1 | −0.295980789 | NA | NA | 3.27E−106 | NA |
| Mup8 | −0.793731191 | −0.829956435 | −0.82718199 | 9.31E−106 | 3.77E−91 |
| Mup12 | −0.589618573 | −1.210517374 | −1.228078411 | 2.77E−105 | 1.10E−243 |
| Aldh1a1 | −0.40245097 | NA | NA | 1.39E−102 | NA |
| Adck3 | −0.482940158 | −0.569692886 | NA | 4.38E−91 | 6.36E−82 |
| Ahcy | −0.375627724 | −0.455985571 | −0.49011253 | 1.82E−89 | 7.46E−73 |
| Cox4i1 | −0.288035158 | −0.57523009 | −0.518813262 | 5.97E−88 | 2.44E−141 |
| Hsd3b7 | −0.451728474 | −0.581642142 | NA | 3.52E−87 | 5.15E−93 |
| Mup2 | −0.650107006 | −0.738721984 | −0.72405868 | 4.29E−84 | 2.14E−98 |
| Rsp14 | −0.346340977 | NA | −0.607507888 | 1.86E−83 | NA |
| Ugt2b36 | −0.287193139 | −0.629671114 | −0.355795521 | 5.82E−81 | 1.10E−145 |
| Acaa1b | −0.263859024 | −0.742478092 | −0.855527427 | 2.20E−79 | 8.89E−175 |
| Psen2 | −0.419555606 | NA | NA | 1.69E−77 | NA |
| Cox6c | −0.41128283 | −0.517417109 | −0.610222852 | 1.20E−74 | 4.59E−79 |
| Gm4076 | −0.342157966 | −0.345826319 | −0.342316728 | 7.98E−74 | 2.87E−41 |
| Uqcrq | −0.316451873 | −0.341869458 | −0.485354968 | 1.28E−72 | 1.11E−51 |
| Igfbp2 | −0.348403589 | −0.576682095 | −0.717832911 | 3.39E−70 | 8.96E−103 |
| Rnf125 | −0.463320018 | −0.423361675 | −0.310633092 | 9.28E−70 | 1.00E−57 |
| mt-Nd6 | −0.515107457 | −0.560268731 | −0.685505467 | 1.57E−69 | 1.14E−72 |
| Rps2 | −0.44250848 | −0.269321392 | −0.474519664 | 4.24E−69 | 2.20E−20 |
| Hsd3b5 | −0.587528886 | −0.886463454 | −0.506259792 | 1.00E−67 | 3.58E−129 |
| Cyp1a2 | −0.481919967 | NA | −0.284567418 | 3.86E−66 | NA |
| Rpl13a | −0.314606263 | NA | −0.480174283 | 7.45E−64 | NA |
| Rps27l | −0.361138678 | −0.377566144 | −0.64582348 | 1.30E−60 | 7.98E−50 |
| Pon1 | −0.302819466 | −0.42825679 | −0.32604762 | 2.10E−60 | 1.97E−64 |
| Gm20594 | −0.559577825 | −0.573781187 | −0.496964367 | 4.48E−60 | 1.21E−52 |
| Mvd | −0.327327327 | NA | NA | 2.22E−59 | NA |
| Sparcl1 | −0.253616986 | −0.253616986 | −0.253616986 | 3.78E−57 | 5.97E−29 |
| Malat1 | −0.652623643 | NA | −0.41172143 | 6.64E−56 | NA |
| Cyp8b1 | −0.364571013 | −0.565483524 | −0.599290325 | 3.21E−55 | 1.09E−90 |
| Ugt2b34 | −0.290735843 | NA | −0.500802828 | 1.26E−52 | NA |
| Hacl1 | −0.332899987 | NA | −0.294916761 | 4.71E−52 | NA |
| Zfp683 | −0.421689112 | −0.421689112 | −0.421689112 | 1.10E−47 | 7.40E−24 |
| Oaf | −0.333766768 | NA | NA | 3.11E−47 | NA |
| Cth | −0.308847665 | −0.332234114 | −0.339209942 | 6.91E−47 | 4.36E−37 |
| Ndufb11 | −0.36792318 | −0.398618793 | −0.479044618 | 4.92E−46 | 7.18E−49 |
| Esd | −0.302906103 | −0.381539462 | −0.338909518 | 9.71E−44 | 1.94E−49 |
| Mpc2 | −0.268047152 | NA | −0.39520967 | 4.77E−43 | NA |
| Ubc | −0.273242853 | NA | NA | 3.58E−41 | NA |
| Hebp1 | −0.313009472 | −0.297464234 | −0.274169133 | 1.26E−40 | 8.29E−32 |
| Gm5096 | −0.399548106 | −0.336704156 | −0.350534749 | 4.99E−39 | 1.27E−33 |
| Oaz1 | −0.339004868 | −0.426286153 | −0.327014451 | 2.14E−37 | 1.27E−51 |
| Cox7b | −0.304638563 | −0.386524219 | −0.650085753 | 6.26E−37 | 1.05E−43 |
| Cyp2c55 | −0.316565473 | NA | −0.347841415 | 6.52E−37 | NA |
| Inmt | −0.308364158 | −0.502414474 | −0.679790467 | 9.17E−37 | 1.61E−65 |
| Cyp2e1 | −0.301497571 | −0.298608744 | 0.296899706 | 4.23E−36 | 2.45E−19 |
| Clpx | −0.306093278 | −0.410840842 | −0.250587235 | 6.23E−36 | 1.11E−53 |
| Ndufb6 | −0.311202811 | −0.479162745 | −0.518384931 | 1.04E−35 | 4.11E−68 |
| Hist1h1c | −0.331045753 | NA | NA | 5.78E−33 | NA |
| Elovl3 | −0.25229278 | −0.812250701 | −0.924845607 | 2.20E−32 | 1.80E−150 |
| Gm10250 | −0.30688429 | −0.304478582 | −0.336847064 | 1.80E−31 | 5.57E−32 |
| Arl4d | −0.328520283 | −0.288941612 | NA | 5.36E−30 | 3.05E−26 |
| Cml1 | −0.277339974 | −0.47269345 | NA | 2.15E−27 | 5.55E−63 |
| Hint2 | −0.299309844 | −0.373773572 | −0.33360366 | 6.95E−27 | 2.60E−44 |
| Aqp8 | −0.331182913 | −0.359788885 | −0.273149234 | 1.22E−25 | 8.19E−45 |
| Ndufa6 | −0.25272382 | −0.330965466 | −0.418444742 | 8.39E−24 | 3.11E−32 |
| Gm9843 | −0.343302667 | −0.298882573 | −0.396322103 | 1.60E−22 | 7.79E−23 |
| Atp5h | −0.351854327 | −0.352362982 | −0.555498653 | 4.84E−19 | 7.17E−29 |
| Dcxr | −0.271339616 | NA | NA | 1.59E−18 | NA |
| Cebpa | −0.260692112 | −0.481611184 | NA | 6.82E−18 | 2.92E−66 |
| Cyp4a10 | −0.289941035 | −0.698160085 | −0.453214203 | 4.30E−17 | 2.61E−98 |
| Sigmar1 | −0.282364617 | NA | NA | 5.67E−17 | NA |
| Nr1i3 | −0.317940692 | NA | NA | 1.11E−16 | NA |
| Ppp2r5a | −0.263513608 | −0.315098874 | −0.281895906 | 3.57E−15 | 3.37E−36 |
| Hhex | −0.283483187 | NA | NA | 9.95E−15 | NA |
| Rpl14 | −0.261157069 | NA | −0.357060958 | 2.50E−13 | NA |
| Tprkb | −0.254084951 | NA | NA | 1.31E−12 | NA |
| Cyp3a59 | −0.263118105 | NA | NA | 3.34E−11 | NA |
| Rps18 | −0.269206741 | NA | −0.415076007 | 5.39E−06 | NA |
| Ftl1 | −0.346327339 | −0.259151442 | −0.347806606 | 3.34E−05 | 1.49E−07 |
| Ubb | −0.263773933 | NA | NA | 0.0138701 | NA |
| Mup1 | −0.490972296 | −0.62326727 | −0.882906956 | 0.022977 | 1.11E−33 |
| Mup7 | −0.293978105 | −0.847726732 | −0.844127621 | 0.067179 | 2.63E−114 |
| Gm24245 | −0.254157233 | −0.264578368 | NA | 1 | 3.33E−13 |
| Mup14 | −0.340890047 | −0.497427273 | −0.741008747 | 1 | 2.38E−27 |
| Apoa2 | NA | −0.615981195 | −0.42773886 | NA | 0 |
| Mup20 | NA | −1.237817166 | −1.081531043 | NA | 0 |
| Car3 | NA | −1.292191881 | −1.168630683 | NA | 2.01E−301 |
| 2810007J24Rik | NA | −1.093998287 | −0.337772907 | NA | 2.63E−273 |
| Tdo2 | 0.25156007 | −0.736504592 | −0.283022598 | 2.99E−135 | 6.44E−267 |
| Rbp4 | NA | −0.580599925 | −0.451760058 | NA | 1.32E−259 |
| Fabp1 | NA | −0.542167351 | −0.826491833 | NA | 8.21E−214 |
| Hsd11b1 | NA | −0.998666341 | −0.332361427 | NA | 3.95E−209 |
| Ces3a | NA | −0.721008165 | NA | NA | 1.41E−206 |
| Hmgcs2 | NA | −0.664269004 | NA | NA | 6.01E−206 |
| Serpina1d | NA | −0.586852756 | NA | NA | 5.86E−199 |
| Selenbp2 | NA | −0.857199402 | −0.354512086 | NA | 9.70E−194 |
| Hrsp12 | NA | −0.800669662 | −0.395396316 | NA | 1.58E−182 |
| Ces3b | NA | −0.819558687 | NA | NA | 6.86E−161 |
| Cyp2d9 | NA | −0.616676578 | −0.3250388 | NA | 8.72E−151 |
| Retsat | NA | −0.784380771 | −0.847260321 | NA | 5.47E−148 |
| Ugt2b1 | NA | −0.722102286 | −0.261758027 | NA | 5.69E−140 |
| Serpina3k | NA | −0.279762031 | −0.298266533 | NA | 3.34E−138 |
| H2-K1 | NA | −0.596584494 | NA | NA | 1.74E−137 |
| Cyp2f2 | 0.275177176 | −0.887034648 | NA | 2.08E−44 | 5.25E−136 |
| Pigr | NA | −0.661452 | NA | NA | 1.08E−134 |
| Rdh7 | NA | −0.496910675 | NA | NA | 5.58E−132 |
| Apoa1 | NA | −0.336207866 | −0.411233725 | NA | 5.20E−127 |
| Apoh | NA | −0.417935459 | NA | NA | 3.98E−126 |
| Aadac | NA | −0.747342823 | −0.466442698 | NA | 6.76E−122 |
| Ephx2 | NA | −0.570800981 | −0.398908219 | NA | 1.49E−121 |
| Gpx1 | NA | −0.321984135 | NA | NA | 2.36E−120 |
| Alb | NA | −0.427719117 | NA | NA | 2.56E−117 |
| Otc | NA | −0.584972427 | NA | NA | 1.50E−116 |
| Apoa5 | 0.30259839 | −0.470514722 | NA | 5.32E−149 | 3.16E−115 |
| Mug1 | NA | −0.497189268 | NA | NA | 3.61E−105 |
| Acsl1 | NA | −0.568030382 | −0.417030322 | NA | 3.63E−104 |
| Cyp4v3 | NA | −0.600614165 | NA | NA | 1.46E−101 |
| Slco1a1 | NA | −0.632477905 | −0.542407867 | NA | 5.52E−101 |
| Cfhr2 | NA | −0.630617674 | −0.428849834 | NA | 7.59E−98 |
| B2m | NA | −0.430851295 | NA | NA | 2.11E−97 |
| Khk | NA | −0.530150378 | NA | NA | 8.89E−91 |
| Apoc1 | NA | −0.31073779 | NA | NA | 1.04E−90 |
| Uox | NA | −0.480422343 | NA | NA | 1.42E−89 |
| Rarres2 | NA | −0.492013911 | NA | NA | 1.76E−89 |
| Serpina12 | NA | −0.612367467 | −0.404557025 | NA | 7.52E−89 |
| Serpinc1 | NA | −0.333556351 | NA | NA | 1.38E−87 |
| Ugt2a3 | NA | −0.54739922 | −0.457903234 | NA | 2.33E−87 |
| Serpinf1 | NA | −0.506061854 | NA | NA | 3.06E−85 |
| Ttr | NA | −0.286138394 | NA | NA | 3.87E−82 |
| C8g | NA | −0.455638561 | NA | NA | 2.41E−80 |
| Serpina1a | NA | −0.35536875 | −0.295262554 | NA | 3.38E−75 |
| Serpina1b | NA | −0.294480791 | NA | NA | 1.99E−74 |
| Serpina1e | NA | −0.308360801 | −0.72519719 | NA | 1.15E−71 |
| Etnppl | NA | −0.549126978 | −0.566620392 | NA | 2.68E−71 |
| 1500017E21Rik | NA | −0.517930222 | NA | NA | 1.93E−69 |
| Atp5f1 | NA | −0.460709866 | −0.428496879 | NA | 1.11E−68 |
| Ces1f | NA | −0.45903711 | −0.533638921 | NA | 9.66E−67 |
| Cyp4a12a | 0.259776426 | −0.4760363 | NA | 1.18E−52 | 1.00E−65 |
| Baat | NA | −0.445833101 | −0.265815179 | NA | 1.01E−65 |
| Fabp2 | NA | −0.488533591 | −0.527186942 | NA | 5.45E−64 |
| Serpina1e | NA | −0.279835146 | NA | NA | 1.24E−63 |
| Sod1 | NA | −0.277853669 | −0.483374172 | NA | 1.68E−63 |
| Cdo1 | NA | −0.343333854 | NA | NA | 2.05E−63 |
| Ech1 | NA | −0.414674796 | −0.504022226 | NA | 2.57E−62 |
| Insig2 | NA | −0.447606183 | −0.275363349 | NA | 4.77E−62 |
| Cox6a1 | NA | −0.390617907 | −0.275276508 | NA | 1.67E−61 |
| Azgp1 | NA | −0.286918536 | NA | NA | 1.16E−60 |
| Ces1c | NA | −0.274525359 | NA | NA | 5.85E−60 |
| Pah | NA | −0.379853874 | NA | NA | 8.65E−60 |
| Lipc | NA | −0.423055461 | NA | NA | 3.19E−59 |
| Dio1 | NA | −0.469732353 | NA | NA | 9.15E−59 |
| Aox3 | 0.275388909 | −0.429460462 | NA | 1.72E−66 | 1.23E−58 |
| Gstm1 | NA | −0.448355325 | −0.35224443 | NA | 3.06E−58 |
| Slc27a5 | NA | −0.374625102 | NA | NA | 1.08E−57 |
| Amy1 | NA | −0.441174 | NA | NA | 1.40E−57 |
| Tmem205 | NA | −0.363762607 | NA | NA | 6.01E−57 |
| Cyp2a12 | NA | −0.396631721 | NA | NA | 6.96E−57 |
| Angptl3 | NA | −0.315601765 | −0.529458826 | NA | 1.53E−55 |
| Cyp7b1 | NA | −0.438158056 | NA | NA | 1.37E−54 |
| Hpd | NA | −0.317943335 | NA | NA | 2.00E−54 |
| Ugp2 | NA | −0.394209561 | NA | NA | 6.59E−54 |
| Ndufa4 | NA | −0.4035957 | −0.512722876 | NA | 1.02E−52 |
| Nudt7 | NA | −0.566043759 | NA | NA | 9.48E−52 |
| Slc10a1 | NA | −0.395488081 | −0.273026083 | NA | 8.15E−51 |
| Scp2 | 0.279911381 | −0.319519472 | NA | 5.16E−159 | 1.15E−50 |
| Sco2 | NA | −0.395951512 | −0.250104753 | NA | 1.04E−49 |
| Cyp2d10 | NA | −0.325332653 | −0.26237727 | NA | 3.76E−49 |
| Chchd2 | NA | −0.369755887 | −0.518624159 | NA | 1.70E−48 |
| Idh3b | NA | −0.360516268 | NA | NA | 4.19E−48 |
| Bckdha | NA | −0.404702898 | NA | NA | 2.27E−47 |
| Glo1 | NA | −0.379233492 | −0.317338548 | NA | 3.51E−47 |
| Sdhb | NA | −0.357362255 | NA | NA | 2.51E−46 |
| Cat | NA | −0.286016441 | NA | NA | 1.00E−45 |
| Ces1e | NA | −0.356936678 | −0.375733204 | NA | 2.06E−45 |
| Cox5b | NA | −0.393691854 | −0.373464063 | NA | 3.39E−45 |
| Atp5g3 | NA | −0.302277876 | −0.357248453 | NA | 2.31E−44 |
| Itm2b | NA | −0.261048932 | NA | NA | 4.20E−44 |
| Etfdh | NA | −0.33708519 | NA | NA | 1.81E−43 |
| Gsto1 | NA | −0.368935961 | NA | NA | 5.15E−43 |
| Hsd3b3 | NA | −0.347460551 | NA | NA | 3.82E−42 |
| Slc27a2 | NA | −0.305294922 | NA | NA | 2.23E−41 |
| Hsd17b6 | NA | −0.36218325 | −0.364685233 | NA | 2.58E−41 |
| Abcd3 | NA | −0.326838469 | NA | NA | 1.45E−40 |
| Mdh1 | NA | −0.329025797 | NA | NA | 2.04E−40 |
| Prdx4 | NA | −0.378024564 | −0.339424675 | NA | 3.39E−40 |
| Hsd17b13 | 0.351593878 | −0.376251609 | NA | 1.14E−64 | 5.32E−40 |
| Proc | NA | −0.319776879 | NA | NA | 1.66E−39 |
| Agxt | NA | −0.303827932 | NA | NA | 1.79E−39 |
| Gls2 | 0.296143664 | −0.353616749 | NA | 1.09E−57 | 2.16E−39 |
| Ugt2b38 | NA | −0.250921219 | NA | NA | 1.20E−38 |
| Ndufb9 | NA | −0.291910914 | −0.483703094 | NA | 2.15E−38 |
| Rabac1 | NA | −0.342063679 | −0.275369587 | NA | 3.25E−38 |
| Slc22a30 | NA | −0.331870745 | −0.25736876 | NA | 7.77E−38 |
| Etfa | NA | −0.33793403 | NA | NA | 8.07E−38 |
| Atp5j | NA | −0.302644835 | −0.415380683 | NA | 4.96E−37 |
| Uqcrfs1 | NA | −0.335765197 | NA | NA | 5.60E−37 |
| Cyp27a1 | NA | −0.326228094 | NA | NA | 1.46E−36 |
| Hal | NA | −0.304647432 | NA | NA | 3.02E−36 |
| Sar1b | NA | −0.301891484 | −0.422088437 | NA | 4.44E−36 |
| Aldh6a1 | NA | −0.30870904 | NA | NA | 4.64E−36 |
| Il1rap | NA | −0.302807638 | NA | NA | 1.94E−35 |
| S100a1 | NA | −0.355544479 | NA | NA | 2.21E−35 |
| Creg1 | NA | −0.258963412 | −0.480113825 | NA | 2.90E−35 |
| 1300017J02Rik | NA | −0.308343818 | NA | NA | 1.20E−34 |
| Acat1 | NA | −0.290805818 | NA | NA | 1.81E−34 |
| Pklr | 0.25984035 | −0.283149611 | NA | 4.04E−64 | 5.43E−34 |
| Dpys | NA | −0.313405671 | NA | NA | 8.84E−34 |
| Aadat | NA | −0.325731706 | NA | NA | 9.13E−34 |
| Cd81 | NA | −0.30883572 | NA | NA | 1.50E−33 |
| Ndufc2 | NA | −0.370889898 | −0.371342649 | NA | 1.56E−33 |
| Bphl | NA | −0.299478155 | NA | NA | 1.70E−33 |
| Qdpr | NA | −0.316595182 | NA | NA | 1.80E−33 |
| Gsta2 | NA | −0.284180552 | NA | NA | 4.56E−33 |
| Acsm3 | NA | −0.280708005 | NA | NA | 5.27E−33 |
| Cyp2d26 | NA | −0.270278674 | NA | NA | 2.04E−32 |
| Cyp2c44 | NA | −0.312764543 | NA | NA | 2.09E−32 |
| Hagh | NA | −0.288430674 | −0.398106396 | NA | 2.99E−32 |
| Ces2a | NA | −0.288140076 | −0.619093582 | NA | 4.30E−32 |
| Gas5 | NA | −0.298417518 | NA | NA | 6.31E−32 |
| Uqcrb | NA | −0.339504918 | −0.359656695 | NA | 1.34E−31 |
| Gm4788 | NA | −0.297042332 | NA | NA | 1.49E−31 |
| Slc2a2 | NA | −0.291267847 | NA | NA | 2.49E−31 |
| Cela1 | NA | −0.252611197 | NA | NA | 1.03E−30 |
| Hgd | NA | −0.273998205 | NA | NA | 2.46E−30 |
| 1110008F13Rik | NA | −0.284458511 | −0.327482854 | NA | 3.77E−30 |
| Csad | NA | −0.308703065 | −1.214863323 | NA | 4.35E−30 |
| Gabarapl1 | NA | −0.297156374 | NA | NA | 2.02E−29 |
| Eci1 | NA | −0.292606814 | −0.31335345 | NA | 4.47E−29 |
| Amdhd1 | NA | −0.272469871 | NA | NA | 8.10E−29 |
| Uqcrh | NA | −0.290259964 | −0.483113531 | NA | 1.52E−28 |
| 0610011F06Rik | NA | −0.28593883 | NA | NA | 3.51E−28 |
| Ftcd | NA | −0.299187456 | NA | NA | 5.47E−28 |
| Cyp4f14 | NA | −0.283565035 | NA | NA | 6.47E−28 |
| Ttc39c | NA | −0.289612311 | NA | NA | 2.34E−27 |
| Ndufv1 | NA | −0.256865664 | NA | NA | 2.43E−27 |
| Pdhb | NA | −0.271619421 | NA | NA | 5.48E−27 |
| Hsd17b11 | NA | −0.272884574 | NA | NA | 8.36E−27 |
| Fcgrt | NA | −0.270945405 | NA | NA | 1.81E−26 |
| Lyrm5 | NA | −0.251171881 | NA | NA | 3.08E−26 |
| Bnip3 | NA | −0.275946132 | −0.272700001 | NA | 3.10E−26 |
| Pygl | NA | −0.25405228 | NA | NA | 1.29E−25 |
| C8b | NA | −0.251084368 | NA | NA | 1.50E−25 |
| Atp5j2 | NA | −0.255051714 | −0.409576712 | NA | 2.55E−25 |
| Fxyd1 | NA | −0.273422293 | NA | NA | 2.95E−25 |
| Nit2 | NA | −0.255321274 | NA | NA | 6.79E−25 |
| Sdha | NA | −0.286156431 | NA | NA | 7.96E−25 |
| Pccb | NA | −0.266663529 | NA | NA | 8.00E−25 |
| Park7 | NA | −0.270929309 | NA | NA | 9.02E−25 |
| Cmbl | NA | −0.265365994 | NA | NA | 9.66E−25 |
| Ces2e | NA | −0.272708804 | NA | NA | 8.71E−24 |
| Cebpb | NA | −0.306330499 | −0.267669957 | NA | 1.73E−23 |
| Pbld1 | NA | −0.264627734 | NA | NA | 2.23E−23 |
| Adipor2 | NA | −0.252551165 | NA | NA | 1.11E−22 |
| Ephx1 | NA | −0.261640077 | −0.332038055 | NA | 1.26E−22 |
| C6 | NA | −0.255102014 | NA | NA | 2.04E−21 |
| Rn7sk | NA | −0.276399379 | NA | NA | 6.19E−14 |
| Hint1 | NA | NA | −0.487016283 | NA | NA |
| Gstp1 | NA | NA | −0.671664785 | NA | NA |
| Gnmt | NA | NA | −0.288175489 | NA | NA |
| Sc4mol | NA | NA | −0.519568919 | NA | NA |
| Prdx1 | NA | NA | −0.353764247 | NA | NA |
| Rpl32 | NA | NA | −0.498592915 | NA | NA |
| Uqcr11 | NA | NA | −0.368070557 | NA | NA |
| Cox7a2 | NA | NA | −0.441101884 | NA | NA |
| Rps3 | NA | NA | −0.339903958 | NA | NA |
| Ndufb8 | NA | NA | −0.389897205 | NA | NA |
| Etfb | NA | NA | −0.270014172 | NA | NA |
| Leap2 | NA | NA | −0.404792721 | NA | NA |
| Uqcr10 | NA | NA | −0.382321788 | NA | NA |
| Akr1c19 | NA | NA | −0.38253326 | NA | NA |
| Ddt | NA | NA | −0.345838673 | NA | NA |
| Eef1b2 | NA | NA | −0.367328377 | NA | NA |
| Chchd10 | NA | NA | −0.28090926 | NA | NA |
| Rps24 | NA | NA | −0.386464837 | NA | NA |
| Acot1 | NA | NA | −0.299327096 | NA | NA |
| Cyp3a44 | NA | NA | −0.297417766 | NA | NA |
| Ndufb4 | NA | NA | −0.331687642 | NA | NA |
| Sdhd | NA | NA | −0.302302961 | NA | NA |
| Rps5 | NA | NA | −0.25674004 | NA | NA |
| Ndufb2 | NA | NA | −0.336855533 | NA | NA |
| Blvrb | NA | NA | −0.351075319 | NA | NA |
| Ndufs6 | NA | NA | −0.341035035 | NA | NA |
| Cnbp | NA | NA | −0.286746587 | NA | NA |
| Cyp4a14 | NA | NA | −0.31778609 | NA | NA |
| Txn1 | NA | NA | −0.277353299 | NA | NA |
| Rpl36al | NA | NA | −0.331844566 | NA | NA |
| Gadd45b | NA | NA | −0.301158726 | NA | NA |
| G0s2 | NA | NA | −0.269113384 | NA | NA |
| Ssr4 | NA | NA | −0.283161199 | NA | NA |
| Minos1 | NA | NA | −0.308403636 | NA | NA |
| Ndufa2 | NA | NA | −0.308306345 | NA | NA |
| Tmem243 | NA | NA | −0.275109242 | NA | NA |
| Rps21 | NA | NA | −0.316866281 | NA | NA |
| Ndufa5 | NA | NA | −0.310280044 | NA | NA |
| Tubb2a | NA | NA | −0.260491764 | NA | NA |
| Akr1c14 | NA | NA | −0.280169437 | NA | NA |
| Atp5o | NA | NA | −0.268645357 | NA | NA |
| Psmb1 | NA | NA | −0.271574562 | NA | NA |
| Rps9 | NA | NA | −0.30861002 | NA | NA |
| Nme1 | NA | NA | −0.291609359 | NA | NA |
| Ndufb5 | NA | NA | −0.274484363 | NA | NA |
| Psma6 | NA | NA | −0.281709731 | NA | NA |
| Rplp1 | NA | NA | −0.274821325 | NA | NA |
| Fam162a | NA | NA | −0.280519821 | NA | NA |
| 4931406C07Rik | NA | NA | −0.28178071 | NA | NA |
| Sc5d | NA | NA | −0.274286355 | NA | NA |
| Timm13 | NA | NA | −0.266267617 | NA | NA |
| Ndufs4 | NA | NA | −0.299960018 | NA | NA |
| Selk | NA | NA | −0.279248941 | NA | NA |
| Mrps18c | NA | NA | −0.27427115 | NA | NA |
| Rpl13 | −0.314606263 | NA | −0.282418264 | 7.45E−64 | NA |
| Rps8 | NA | NA | −0.279845708 | NA | NA |
| Rps15 | NA | NA | −0.324542895 | NA | NA |
| N1rp12 | NA | NA | −0.269307598 | NA | NA |
| Timm8b | NA | NA | −0.25098905 | NA | NA |
| Dhrs4 | NA | NA | −0.268862728 | NA | NA |
| Rps10 | NA | NA | −0.252659935 | NA | NA |
| Rpl34 | NA | NA | −0.264586248 | NA | NA |
| Hspe1 | NA | NA | −0.257910168 | NA | NA |
| Usmg5 | NA | NA | −0.28365494 | NA | NA |
| Rpl26 | NA | NA | −0.264487941 | NA | NA |
| Rps19 | NA | NA | −0.25849266 | NA | NA |

| ID | PH120p | avg_logFC | p_val_adj |
|---|---|---|---|
| Saa2 | 0 | 4.43818759 | 0 |
| Mt1 | 0 | 2.610854994 | 0 |
| Mt2 | 0 | 2.6060431 | 0 |
| Saa1 | 0 | 3.650785996 | 0 |
| Steap4 | 1.31E−284 | 1.890015167 | 0 |
| Serpina3n | 0 | 1.759758939 | 0 |
| Lcn2 | 0 | 2.39758181 | 0 |
| Lpin1 | 4.53E−64 | 1.327657625 | 0 |
| n-R5-8s1 | 0 | 1.289598504 | 0 |
| Igfbp1 | 1.15E−78 | 1.288789902 | 0 |
| Cxcl1 | NA | 1.230398019 | 0 |
| Scd1 | 3.71E−204 | 1.081676999 | 0 |
| Gm26924 | 0 | 1.445355764 | 0 |
| Il1r1 | 4.69E−177 | 0.998168131 | 0 |
| Fga | 0 | 1.039432587 | 0 |
| Gm24601 | 0 | 0.924568273 | 0 |
| Nnmt | 6.69E−98 | 0.901052904 | 0 |
| Itih4 | 0 | 1.18011817 | 0 |
| St3gal5 | NA | 0.882761517 | 0 |
| Tacc2 | NA | 0.877623973 | 0 |
| Lrg1 | 0 | 1.260509735 | 0 |
| Fam134b | NA | 0.865578177 | 0 |
| Tat | 3.93E−194 | 0.857965136 | 0 |
| Saa4 | 0 | 1.158596154 | 0 |
| Thrsp | 4.31E−54 | 0.836598219 | 0 |
| Slc39a14 | 1.16E−108 | 0.831584936 | 0 |
| Lars2 | 0 | 1.419129056 | 0 |
| Fgg | 0 | 0.809322713 | 0 |
| Slc38a2 | 1.34E−44 | 0.797899442 | 0 |
| Serpina3m | 2.80E−48 | 0.779052745 | 0 |
| Hp | 0 | 1.584889895 | 0 |
| Fgb | 0 | 0.739519125 | 0 |
| Gm15564 | 0 | 1.485885928 | 0 |
| Serpina10 | 1.35E−230 | 0.871044179 | 0 |
| Plin2 | NA | 0.720463076 | 0 |
| Fmo5 | 5.95E−73 | 0.690160801 | 0 |
| Fkbp5 | NA | 0.663304836 | 0 |
| Slc25a47 | NA | 0.635928777 | 0 |
| Mat1a | 2.90E−246 | 0.623734998 | 0 |
| Acox1 | 1.80E−68 | 0.540022158 | 0 |
| Aldob | NA | 0.490200848 | 0 |
| C3 | 0 | 1.071950932 | 0 |
| B3galt1 | 8.61E−40 | 0.788734938 | 3.52E−301 |
| Fn1 | 0 | 1.018772906 | 0 |
| Hnf4a | 3.08E−110 | 0.644195253 | 5.68E−290 |
| Btg2 | 2.69E−68 | 0.772686832 | 7.24E−290 |
| H3f3b | NA | 0.755567107 | 4.44E−288 |
| Aass | 3.70E−47 | 0.705615345 | 1.07E−286 |
| Sephs2 | 1.86E−182 | 0.618707003 | 1.06E−281 |
| Cldn14 | NA | 0.511583526 | 4.32E−281 |
| Rcan1 | NA | 0.509239866 | 4.58E−279 |
| Hspb8 | 4.23E−133 | 0.667847253 | 9.19E−267 |
| Arhgef26 | 4.52E−47 | 0.568544702 | 4.92E−265 |
| Slc7a2 | 2.68E−62 | 0.622326529 | 6.74E−265 |
| Pfkfb3 | NA | 0.426580733 | 3.82E−264 |
| Pck1 | 8.65E−210 | 0.495042924 | 4.86E−264 |
| Errfi1 | NA | 0.500850526 | 7.43E−262 |
| Gjb2 | 1.88E−70 | 0.639507436 | 9.92E−255 |
| Snx10 | NA | 0.558244633 | 1.59E−251 |
| Bcl3 | NA | 0.419650143 | 3.05E−251 |
| Hpx | 0 | 1.443685235 | 0 |
| Bach1 | NA | 0.533237374 | 5.54E−247 |
| Tifa | 4.02E−144 | 0.392339477 | 3.72E−241 |
| 2010003K11Rik | NA | 0.372219971 | 1.06E−239 |
| Pzp | 0 | 0.636851805 | 0 |
| Tiparp | NA | 0.489122153 | 7.07E−236 |
| Rapgef4 | NA | 0.588317735 | 4.64E−233 |
| Mup10 | 4.03E−105 | 0.428268029 | 1.14E−231 |
| Egfr | 4.26E−199 | 0.65440823 | 5.47E−228 |
| mmu-mir-6236 | 5.14E−284 | 1.257248389 | 5.14E−284 |
| Etnk2 | NA | 0.592353201 | 7.87E−221 |
| Mfsd2a | NA | 0.496351884 | 1.64E−219 |
| Txnip | NA | 0.552612237 | 1.04E−218 |
| Arg1 | NA | 0.381616351 | 2.23E−218 |
| Gpt2 | NA | 0.533331785 | 3.67E−218 |
| Slc25a25 | 2.18E−84 | 0.544465891 | 2.55E−217 |
| Actb | NA | 0.456981443 | 2.15E−210 |
| Xbp1 | NA | 0.539986 | 1.34E−208 |
| Upp2 | NA | 0.541753512 | 2.02E−206 |
| Fbf1 | NA | 0.298573909 | 4.53E−206 |
| Plin5 | NA | 0.489257529 | 1.01E−205 |
| Plk3 | NA | 0.488016367 | 8.22E−205 |
| Trib1 | NA | 0.581754281 | 1.57E−204 |
| Mtss1 | 1.12E−54 | 0.552137981 | 2.07E−204 |
| Klf6 | NA | 0.3616937 | 8.00E−203 |
| Itih3 | 0 | 1.223022315 | 0 |
| Slc10a2 | NA | 0.30563824 | 6.54E−197 |
| Sdc4 | 2.10E−74 | 0.43560504 | 2.70E−193 |
| Atf3 | 3.11E−27 | 0.597274889 | 1.23E−191 |
| Ptp4a1 | NA | 0.393137367 | 6.10E−190 |
| Prg4 | 8.36E−244 | 0.810835665 | 8.36E−244 |
| Rgs16 | NA | 0.438105488 | 3.55E−188 |
| Gpcpd1 | NA | 0.53780646 | 5.36E−188 |
| Rhob | NA | 0.37207281 | 4.39E−187 |
| Glul | 0.000261488 | 1 | 1.01E−186 |
| Fam35a | NA | 0.470461753 | 7.67E−186 |
| Klf10 | NA | 0.304779554 | 1.06E−185 |
| Egr1 | 3.36E−20 | 0.620480469 | 7.27E−185 |
| Apcs | 5.94E−275 | 2.049485412 | 0 |
| Shb | NA | 0.430613558 | 4.03E−182 |
| Orm1 | 0 | 1.823760769 | 0 |
| Fgl1 | 0 | 1.561880768 | 0 |
| 1810055G02Rik | NA | 0.382584093 | 7.73E−175 |
| Stat3 | 1.75E−98 | 0.483904634 | 3.58E−174 |
| Abca1 | 2.16E−52 | 0.50506234 | 3.19E−172 |
| Igfbp4 | 1.27E−128 | 0.411174683 | 6.69E−171 |
| Got1 | 7.35E−48 | 0.542891511 | 7.82E−171 |
| Cdkn1a | NA | 0.449325471 | 7.06E−169 |
| Apob | 4.63E−151 | 0.505397578 | 8.05E−169 |
| Gfra1 | NA | 0.474694426 | 4.73E−168 |
| Ivns1abp | NA | 0.46254094 | 9.44E−168 |
| Slc41a2 | 4.36E−127 | 0.441321771 | 2.22E−166 |
| Slc25a51 | NA | 0.372157664 | 1.11E−164 |
| Myo1e | NA | 0.399456672 | 7.83E−162 |
| Gm23935 | 1.38E−297 | 1.207244314 | 1.38E−297 |
| Bhlhe40 | 1.23E−69 | 0.403249298 | 3.04E−158 |
| Ddx3x | NA | 0.47100162 | 1.36E−155 |
| Tmem87b | NA | 0.288330587 | 8.72E−155 |
| Dak | NA | 0.481947664 | 1.71E−153 |
| Slc25a22 | NA | 0.390931532 | 7.19E−153 |
| Fasn | NA | 0.467168076 | 2.55E−152 |
| Fh1 | NA | 0.471428771 | 7.97E−152 |
| Rnf103 | NA | 0.438540394 | 1.39E−149 |
| Sds | NA | 0.787094904 | 5.50E−149 |
| B4galt1 | NA | 0.321380868 | 4.77E−146 |
| Cps1 | 8.39E−77 | 0.336429448 | 1.19E−145 |
| Atp1b1 | NA | 0.424801231 | 1.32E−145 |
| Jmjd1c | NA | 0.39612601 | 1.05E−144 |
| Gm26917 | 7.15E−51 | 0.475554164 | 2.42E−144 |
| Junb | NA | 0.458500682 | 1.57E−143 |
| 4933426M11Rik | NA | 0.28624304 | 1.78E−142 |
| Pik3r1 | NA | 0.389193614 | 1.89E−142 |
| D10Wsu102e | NA | 0.256022156 | 1.03E−141 |
| Hyou1 | 3.39E−91 | 0.496156079 | 3.60E−141 |
| Glud1 | 1.30E−69 | 0.359099708 | 1.11E−139 |
| Cpt1a | 1.19E−34 | 0.42860734 | 3.31E−139 |
| Epas1 | NA | 0.38425941 | 2.12E−138 |
| Jun | NA | 0.479246114 | 2.64E−135 |
| Mbd1 | NA | 0.348094524 | 2.44E−134 |
| Rnd3 | NA | 0.285147682 | 4.66E−134 |
| Litaf | NA | 0.396007707 | 2.34E−132 |
| Ptpn1 | NA | 0.268263225 | 6.92E−130 |
| Bcl2l1 | NA | 0.280315334 | 1.17E−128 |
| Grn | 1.12E−47 | 0.403315761 | 3.44E−128 |
| Tns1 | NA | 0.33981795 | 4.58E−128 |
| Slc25a15 | 4.09E−89 | 0.402049896 | 6.99E−128 |
| Gadd45g | NA | 0.392031882 | 7.78E−128 |
| Wbp1l | NA | 0.409138156 | 1.29E−127 |
| Foxa3 | NA | 0.307992015 | 2.28E−126 |
| Myh9 | 2.83E−141 | 0.631035657 | 2.83E−141 |
| Fos | NA | 0.524733327 | 3.55E−125 |
| Id2 | NA | 0.430147785 | 5.03E−124 |
| Itih2 | NA | 0.336714211 | 1.56E−122 |
| Plg | 0 | 0.596524395 | 0 |
| Qsox1 | 2.24E−241 | 0.725062115 | 2.24E−241 |
| Ccnl1 | NA | 0.341338448 | 1.67E−120 |
| Eef1a1 | NA | 0.340768186 | 6.14E−119 |
| Ier2 | NA | 0.452598264 | 1.60E−118 |
| Elovl6 | NA | 0.37962641 | 5.92E−117 |
| Nedd4l | NA | 0.271827098 | 1.51E−116 |
| Ppip5k2 | NA | 0.30299148 | 3.63E−115 |
| Epb4.1 | NA | 0.349430673 | 1.93E−113 |
| Cfh | 0 | 0.83288399 | 0 |
| Ulk1 | NA | 0.297841785 | 5.13E−113 |
| Ell2 | NA | 0.371745977 | 9.30E−113 |
| Tnfrsf1a | 1.90E−43 | 0.346845029 | 3.42E−112 |
| Eif4g2 | 8.07E−39 | 0.384302798 | 2.51E−110 |
| Ly6e | 0 | 1.093957206 | 0 |
| Il6ra | NA | 0.251696836 | 2.93E−108 |
| Lpgat1 | 1.05E−64 | 0.400787984 | 1.25E−107 |
| Fads2 | NA | 0.323744791 | 4.78E−106 |
| Atl2 | NA | 0.321873809 | 5.02E−106 |
| Dst | NA | 0.329905553 | 7.40E−105 |
| Jak1 | NA | 0.362766687 | 2.44E−103 |
| Nfil3 | NA | 0.338730712 | 5.23E−103 |
| Elovl5 | NA | 0.382267188 | 1.08E−102 |
| Mgll | NA | 0.321840174 | 6.61E−101 |
| Sult1a1 | NA | 0.371023537 | 2.67E−99 |
| Man2a1 | 1.19E−46 | 0.369999358 | 3.60E−98 |
| Eef2 | 1.02E−72 | 0.321705764 | 1.70E−97 |
| Aff4 | NA | 0.344186222 | 7.00E−97 |
| Palmd | NA | 0.284673594 | 9.43E−97 |
| Zfp36 | NA | 0.407014847 | 1.05E−96 |
| Eif1a | NA | 0.295219945 | 7.80E−96 |
| Slc3a1 | 2.59E−55 | 0.381791335 | 1.93E−94 |
| Ifitm3 | 2.86E−148 | 0.506857449 | 2.86E−148 |
| Atp2a2 | 4.42E−29 | 0.350363266 | 8.21E−94 |
| Nop58 | NA | 0.257566653 | 1.12E−93 |
| Enpp2 | NA | 0.316882991 | 4.48E−93 |
| Gm2a | NA | 0.341145926 | 6.61E−93 |
| Cyb5r3 | NA | 0.337684684 | 1.90E−92 |
| Apoa4 | NA | 1.186863065 | 3.01E−137 |
| Aldh1l1 | 5.65E−221 | 0.564932962 | 5.65E−221 |
| Eci2 | NA | 0.288159513 | 5.23E−88 |
| Slc38a3 | 4.90E−80 | 0.353500212 | 1.28E−87 |
| Dusp1 | NA | 0.300628481 | 1.26E−86 |
| Igf1 | NA | 0.277138761 | 2.86E−86 |
| Serinc3 | NA | 0.293964974 | 2.71E−85 |
| Tgm2 | 2.52E−59 | 0.37123494 | 5.50E−85 |
| C8a | 3.00E−96 | 0.404198047 | 3.00E−96 |
| Txnrd1 | NA | 0.314979852 | 3.22E−84 |
| Asl | NA | 0.297673772 | 1.76E−83 |
| Por | NA | 0.290745567 | 3.93E−83 |
| Sec14l4 | NA | 0.285169227 | 5.54E−83 |
| Uroc1 | 3.99E−76 | 0.459322934 | 1.13E−82 |
| Lrp6 | NA | 0.270740438 | 1.18E−82 |
| Atf6 | NA | 0.280521162 | 1.55E−82 |
| Ddi2 | NA | 0.302519699 | 2.71E−82 |
| Dstn | NA | 0.271381717 | 2.90E−82 |
| Id3 | NA | 0.399520373 | 9.94E−82 |
| H6pd | 1.01E−111 | 0.491965789 | 1.01E−111 |
| Xdh | 1.44E−68 | 0.373181784 | 8.54E−81 |
| Tmed5 | NA | 0.296018777 | 1.36E−80 |
| G6pc | NA | 0.341403933 | 2.15E−80 |
| Mttp | NA | 0.296651415 | 2.14E−79 |
| Angptl4 | NA | 0.315315425 | 2.44E−79 |
| Nrp1 | NA | 0.268796793 | 7.49E−79 |
| Fgf1 | NA | 0.287983029 | 7.97E−79 |
| Prox1 | NA | 0.31520499 | 3.03E−77 |
| Gck | NA | 0.260705425 | 6.82E−77 |
| Picalm | NA | 0.276902165 | 1.27E−76 |
| Lbp | 1.44E−171 | 0.656662588 | 1.44E−171 |
| Azin1 | NA | 0.268891169 | 7.99E−76 |
| Ddx5 | NA | 0.251675045 | 3.02E−75 |
| Cpb2 | 3.34E−100 | 0.403790403 | 3.34E−100 |
| Il6st | 3.35E−58 | 0.382881893 | 3.02E−73 |
| Rmnd5a | NA | 0.273473023 | 5.48E−73 |
| Trp53inp1 | NA | 0.269278401 | 1.01E−72 |
| Ctsb | 4.51E−149 | 0.553974491 | 4.51E−149 |
| Acsl4 | NA | 0.273275871 | 5.43E−70 |
| Elovl2 | NA | 0.285038606 | 2.60E−69 |
| Iqgap2 | NA | 0.283192936 | 5.91E−69 |
| Lrp1 | 2.17E−231 | 0.914046975 | 2.17E−231 |
| Arhgef12 | 1.26E−32 | 0.304865834 | 1.49E−67 |
| Pim3 | NA | 0.280137371 | 3.27E−67 |
| C4bp | 1.14E−148 | 0.530516995 | 1.14E−148 |
| Lrpprc | NA | 0.257016288 | 6.41E−67 |
| Atp1a1 | NA | 0.261718912 | 2.96E−65 |
| Copa | 4.75E−40 | 0.294256247 | 1.69E−64 |
| Acly | NA | 0.287663867 | 4.67E−64 |
| Rcl1 | NA | 0.273905375 | 1.37E−63 |
| Alas1 | 2.11E−43 | 0.423999349 | 6.82E−63 |
| Kmo | 4.54E−49 | 0.305370487 | 2.45E−62 |
| Ppp1r3b | NA | 0.259418401 | 1.58E−61 |
| Zfp36l1 | NA | 0.271823824 | 4.54E−61 |
| Efna1 | 2.85E−25 | 0.268455179 | 2.55E−60 |
| Ctsl | NA | 0.256520866 | 1.56E−59 |
| Ptplad1 | NA | 0.255273084 | 2.04E−59 |
| Hdlbp | 2.52E−71 | 0.394030651 | 2.52E−71 |
| Pnpla7 | NA | 0.251226201 | 4.93E−58 |
| Fads1 | NA | 0.267264464 | 2.35E−57 |
| Nedd4 | NA | 0.259978699 | 7.35E−56 |
| Mtus1 | 4.23E−49 | 0.385483144 | 2.92E−54 |
| Cyp2c70 | 6.40E−150 | 0.511802822 | 6.40E−150 |
| Brd2 | NA | 0.258446853 | 8.84E−51 |
| Saa3 | NA | 2.199786317 | 0 |
| Orm2 | 0 | 2.405086745 | 0 |
| Ambp | 6.84E−288 | 0.496213815 | 6.84E−288 |
| Trf | NA | 0.436833817 | 1.60E−148 |
| Rnase4 | 0 | 1.031156304 | 0 |
| Prtn3 | 4.48E−205 | 0.350182853 | 4.48E−205 |
| Fam25c | NA | 0.96762143 | 2.76E−118 |
| Cp | 5.81E−290 | 0.655511152 | 5.81E−290 |
| Dhcr24 | 1.45E−130 | 0.759730578 | 1.45E−130 |
| Kng1 | 0 | 0.546835494 | 0 |
| Csf2ra | NA | 0.342712543 | 3.48E−100 |
| D17H6S56E-5 | NA | 0.560241333 | 2.13E−95 |
| Cxcl14 | NA | 0.268462966 | 7.87E−89 |
| Hsp90ab1 | 1.17E−94 | 0.455851506 | 1.17E−94 |
| Tmem176a | 1.27E−190 | 0.675060004 | 1.27E−190 |
| Isyna1 | 2.67E−89 | 0.320415943 | 2.67E−89 |
| Cd5l | 4.68E−138 | 0.49371428 | 4.68E−138 |
| Ccnd1 | NA | 0.385849227 | 6.27E−78 |
| P4hb | 1.31E−132 | 0.38506968 | 1.31E−132 |
| Tmem176b | 3.22E−226 | 0.713749974 | 3.22E−226 |
| Gas6 | 1.89E−110 | 0.35878079 | 1.89E−110 |
| Hmgcs1 | 6.89E−53 | 0.702526059 | 4.05E−72 |
| Ang | 3.32E−128 | 0.644516227 | 3.32E−128 |
| Marco | NA | 0.30539135 | 1.17E−70 |
| Ahsg | 3.27E−222 | 0.32482682 | 3.27E−222 |
| Gm12744 | NA | 0.350643059 | 1.69E−65 |
| Clu | 0 | 0.745288027 | 0 |
| Rbp1 | 1.56E−104 | 0.396801127 | 1.56E−104 |
| C1qb | 6.35E−100 | 0.361309767 | 6.35E−100 |
| Mki67 | NA | 0.39305852 | 1.59E−57 |
| Acnat2 | NA | 0.33547101 | 1.88E−56 |
| Top2a | NA | 0.33486876 | 1.10E−54 |
| Clec4f | 1.73E−119 | 0.487336922 | 1.73E−119 |
| Rpl41 | NA | 0.444557582 | 1.56E−51 |
| Cdca3 | NA | 0.286393603 | 1.87E−51 |
| Ifitm2 | 8.05E−44 | 0.526891524 | 1.07E−50 |
| Dbi | NA | 0.360631658 | 9.49E−48 |
| 1100001G20Rik | NA | 0.310291029 | 4.13E−43 |
| Cdc20 | NA | 0.272554242 | 3.12E−38 |
| Cd302 | NA | 0.384976006 | 1.82E−37 |
| Csf1r | NA | 0.284684372 | 3.79E−37 |
| Gstm2 | NA | 0.466218536 | 3.99E−35 |
| Apoc2 | NA | 0.517142972 | 6.48E−35 |
| Krt18 | NA | 0.520354076 | 5.88E−34 |
| Tmsb4x | 1.30E−70 | 0.347657914 | 1.30E−70 |
| Dnajc12 | NA | 0.312907586 | 2.54E−30 |
| Lyz2 | 3.33E−93 | 0.402735723 | 3.33E−93 |
| BC048546 | NA | 0.287060939 | 1.16E−27 |
| Gstm3 | NA | 0.793361403 | 1.46E−26 |
| Hspa8 | NA | 0.302152434 | 4.31E−21 |
| Dynll1 | NA | 0.289016997 | 1.79E−19 |
| Tspan4 | NA | 0.32084381 | 3.41E−19 |
| Kng2 | 1.89E−81 | 0.368809413 | 1.89E−81 |
| 2200002D01Rik | NA | 0.257614019 | 1.71E−17 |
| Cyp51 | 5.74E−31 | 0.40339747 | 5.74E−31 |
| Cyp7a1 | NA | 0.598824806 | 5.91E−17 |
| Mlec | NA | 0.400321297 | 1.46E−15 |
| Smc4 | NA | 0.338338192 | 3.80E−14 |
| Hrg | 3.97E−209 | 0.58732025 | 3.97E−209 |
| Sdc1 | NA | 0.330139692 | 1.43E−12 |
| Hspa5 | 1.02E−98 | 0.339769129 | 1.02E−98 |
| Dap | 5.49E−103 | 0.465257779 | 5.49E−103 |
| Hsph1 | 1.92E−73 | 0.425977247 | 1.92E−73 |
| Mrap | NA | 0.280974611 | 1.04E−10 |
| Creld2 | 5.90E−44 | 0.362477611 | 5.90E−44 |
| Cpox | NA | 0.294319953 | 2.98E−08 |
| Ebp | NA | 0.252819864 | 3.69E−07 |
| Fdps | 7.67E−24 | 0.303232732 | 7.67E−24 |
| Ugdh | NA | 0.260443532 | 4.68E−05 |
| Tmpo | NA | 0.2573532 | 5.30E−05 |
| Lss | NA | 0.294433908 | 0.00013007 |
| Akr1d1 | NA | 0.258293189 | 0.00030931 |
| Snord13 | NA | 0.408788625 | 0.00032705 |
| Lsr | NA | 0.262580416 | 0.00577047 |
| Tuba1c | NA | 0.302939755 | 0.00605691 |
| Caprin1 | 2.84E−37 | 0.303020851 | 2.84E−37 |
| Atl3 | NA | 0.255322039 | 0.02112341 |
| Manf | NA | 0.282950631 | 0.02326868 |
| Avpi1 | NA | 0.30186919 | 0.10146619 |
| Krt8 | NA | 0.252276759 | 0.18282921 |
| Sqle | NA | 1 | 0.27714068 |
| Nucks1 | NA | 1 | 0.2710557 |
| Lect2 | NA | 1 | 1.07E−19 |
| Oat | NA | 1 | 4.82E−23 |
| Tubb5 | NA | 1 | 0.31382452 |
| Tubb4b | NA | 1 | 0.28149027 |
| C4b | 0 | 0.820022988 | 0 |
| Vtn | 8.96E−294 | 0.516935534 | 8.96E−294 |
| Hc | 2.31E−207 | 0.611232959 | 2.31E−207 |
| Orm3 | 1.10E−191 | 0.517250103 | 1.10E−191 |
| Serping1 | 3.54E−179 | 0.535830748 | 3.54E−179 |
| Mpeg1 | 4.24E−174 | 0.541170007 | 4.24E−174 |
| Cml2 | 7.24E−143 | 0.486040271 | 7.24E−143 |
| Itih1 | 1.36E−141 | 0.496617676 | 1.36E−141 |
| Comt | 2.17E−123 | 0.384473508 | 2.17E−123 |
| Calr | 1.61E−116 | 0.391955529 | 1.61E−116 |
| Scara5 | 2.62E−114 | 0.285535495 | 2.62E−114 |
| Cfi | 2.16E−113 | 0.390834066 | 2.16E−113 |
| Mug2 | 3.40E−113 | 0.536577024 | 3.40E−113 |
| H2-D1 | 1.14E−105 | 0.433183706 | 1.14E−105 |
| Lpl | 1.77E−105 | 0.355011922 | 1.77E−105 |
| Col18a1 | 5.95E−103 | 0.459095159 | 5.95E−103 |
| Wfdc17 | 1.32E−101 | 0.317329894 | 1.32E−101 |
| Fetub | 1.86E−98 | 0.425407474 | 1.86E−98 |
| Serpinf2 | 7.60E−88 | 0.361403772 | 7.60E−88 |
| H2-Q10 | 4.52E−87 | 0.250632773 | 4.52E−87 |
| Aldh2 | 7.35E−82 | 0.296492218 | 7.35E−82 |
| Cyp3a13 | 6.41E−81 | 0.420680082 | 6.41E−81 |
| F5 | 1.06E−75 | 0.436709177 | 1.06E−75 |
| Ldhd | 1.04E−74 | 0.387086477 | 1.04E−74 |
| Pcx | 3.33E−72 | 0.367136085 | 3.33E−72 |
| Fah | 1.51E−71 | 0.325518495 | 1.51E−71 |
| Abhd2 | 1.30E−66 | 0.267241512 | 1.30E−66 |
| Ugt1a1 | 5.30E−66 | 0.370092558 | 5.30E−66 |
| Cyr61 | 9.66E−64 | 0.302881921 | 9.66E−64 |
| Pros1 | 3.01E−60 | 0.26812884 | 3.01E−60 |
| Slc22a7 | 1.44E−59 | 0.307857498 | 1.44E−59 |
| Ctss | 2.68E−57 | 0.270183132 | 2.68E−57 |
| P1xnb2 | 4.58E−57 | 0.381208682 | 4.58E−57 |
| Gne | 4.60E−57 | 0.355262513 | 4.60E−57 |
| Abcb11 | 5.21E−57 | 0.351857633 | 5.21E−57 |
| Cpn2 | 6.01E−57 | 0.349996687 | 6.01E−57 |
| Lgals9 | 3.63E−55 | 0.327550674 | 3.63E−55 |
| Hamp2 | 1.91E−54 | 0.337201585 | 1.91E−54 |
| Psap | 5.30E−54 | 0.318472244 | 5.30E−54 |
| Mafb | 1.15E−53 | 0.356246663 | 1.15E−53 |
| Cd36 | 2.36E−53 | 0.291074527 | 2.36E−53 |
| Pdia3 | 3.36E−53 | 0.276156858 | 3.36E−53 |
| Habp2 | 3.50E−53 | 0.313462443 | 3.50E−53 |
| Apon | 2.92E−52 | 0.318989204 | 2.92E−52 |
| Nbr1 | 4.73E−52 | 0.337157409 | 4.73E−52 |
| Slc25a23 | 4.31E−51 | 0.332651576 | 4.31E−51 |
| F9 | 1.74E−49 | 0.303980935 | 1.74E−49 |
| Iigp1 | 4.95E−49 | 0.305889089 | 4.95E−49 |
| Agt | 4.95E−49 | 0.258205872 | 4.95E−49 |
| Gns | 9.33E−49 | 0.323226146 | 9.33E−49 |
| Bsg | 5.75E−48 | 0.273171859 | 5.75E−48 |
| Scarb1 | 6.10E−48 | 0.32724782 | 6.10E−48 |
| Sardh | 2.96E−45 | 0.28626336 | 2.96E−45 |
| Edem1 | 1.82E−44 | 0.301212478 | 1.82E−44 |
| Abat | 2.42E−44 | 0.305043176 | 2.42E−44 |
| Dpyd | 9.45E−44 | 0.315635393 | 9.45E−44 |
| Myo18a | 1.29E−42 | 0.253700048 | 1.29E−42 |
| St6gal1 | 2.92E−42 | 0.295334926 | 2.92E−42 |
| Tapbp | 2.77E−41 | 0.265071778 | 2.77E−41 |
| Ifih1 | 1.00E−40 | 0.264296168 | 1.00E−40 |
| BC025446 | 4.46E−39 | 0.266911384 | 4.46E−39 |
| Pcdh1 | 7.89E−39 | 0.265458741 | 7.89E−39 |
| 1300002K09Rik | 1.78E−37 | 0.30148927 | 1.78E−37 |
| Plec | 2.04E−37 | 0.267122123 | 2.04E−37 |
| Uba1 | 1.91E−36 | 0.281371963 | 1.91E−36 |
| Kynu | 2.94E−36 | 0.28095477 | 2.94E−36 |
| Rxra | 5.74E−36 | 0.264560061 | 5.74E−36 |
| Tfr2 | 8.48E−36 | 0.268628506 | 8.48E−36 |
| Ctnnd1 | 1.41E−35 | 0.252846161 | 1.41E−35 |
| Fndc3a | 7.28E−35 | 0.273177056 | 7.28E−35 |
| Adh4 | 1.86E−34 | 0.282357284 | 1.86E−34 |
| C130074G19Rik | 3.22E−33 | 0.255729195 | 3.22E−33 |
| Ptprf | 4.49E−33 | 0.27768605 | 4.49E−33 |
| Osbp19 | 1.06E−31 | 0.270713808 | 1.06E−31 |
| Vwa8 | 1.52E−31 | 0.262040545 | 1.52E−31 |
| Plcxd2 | 6.38E−31 | 0.266285463 | 6.38E−31 |
| Serpina6 | 1.21E−30 | 0.269623074 | 1.21E−30 |
| Mbl2 | 6.49E−30 | 0.268002193 | 5.49E−35 |
| Pid1 | 2.30E−28 | 0.263310286 | 2.30E−28 |
| Cmah | 6.55E−28 | 0.251319774 | 6.55E−28 |
| Aldh1b1 | 1.25E−25 | 0.360480753 | 1.25E−25 |
| Rgn | 2.71E−20 | 0.261094268 | 2.71E−20 |
| Ifi27 | 5.38E−18 | 0.274586258 | 5.38E−18 |
| Gulo | 3.90E−10 | 0.282891961 | 3.90E−10 |
| Slc1a2 | 0.006115724 | 0.262926395 | 0.00611572 |
| Mup3 | 0 | −1.423759103 | 0 |
| Cyb5 | 2.13E−288 | −0.751689441 | 0 |
| mt-Rnr1 | 0 | −0.872186844 | 0 |
| mt-Rnr2 | 0 | −0.967453486 | 0 |
| Mup11 | 0 | −1.769206667 | 0 |
| mt-Nd2 | 0 | −1.350784061 | 0 |
| mt-Nd1 | 0 | −1.543996447 | 0 |
| mt-Nd5 | 0 | −1.49645814 | 0 |
| Gm13775 | 5.37E−288 | −1.255657303 | 0 |
| mt-Cytb | 0 | −1.554026854 | 0 |
| mt-Co2 | 1.21E−274 | −1.417328219 | 0 |
| mt-Co1 | 0 | −1.569950123 | 0 |
| Mup5 | 0 | −1.774609558 | 0 |
| mt-Nd4 | 0 | −1.953205815 | 0 |
| Mup4 | 0 | −1.945646142 | 0 |
| Mup6 | 0 | −2.645373468 | 0 |
| Mup9 | 0 | −2.520658851 | 0 |
| Mup17 | 0 | −2.514472885 | 0 |
| Mup18 | 0 | −2.617739049 | 0 |
| Mup19 | 0 | −3.632170505 | 0 |
| mt-Co3 | 3.03E−229 | −1.233961828 | 1.47E−287 |
| Cyp2c29 | 3.62E−184 | −1.027267705 | 2.76E−235 |
| Mup16 | 0 | −1.245137265 | 0 |
| Cyp2b10 | 1.24E−168 | −1.254972098 | 1.74E−217 |
| Ankrd55 | 4.09E−185 | −1.950942364 | 2.52E−247 |
| Ugt2b5 | 5.77E−289 | −0.716336886 | 5.77E−289 |
| Apoe | NA | −0.615882949 | 0 |
| Gsta3 | 3.08E−250 | −0.796926805 | 3.08E−250 |
| Cyp2c50 | 2.38E−78 | −0.763912176 | 8.52E−186 |
| Mup21 | 0 | −1.637812283 | 0 |
| Cyp2c54 | 1.42E−114 | −0.712859584 | 1.75E−163 |
| Akr1c6 | 3.18E−123 | −0.501143524 | 6.85E−150 |
| Apoc4 | 1.19E−182 | −0.501759591 | 1.19E−182 |
| mt-Atp6 | 3.13E−134 | −0.914635453 | 7.98E−137 |
| Mup15 | 5.96E−268 | −1.364182598 | 5.96E−268 |
| Cyp3a11 | 0 | −0.803553063 | 0 |
| Mgst1 | 2.20E−129 | −0.36875402 | 2.50E−131 |
| Cyp3a25 | 4.60E−99 | −0.416456818 | 5.44E−129 |
| Ces1d | 0 | −1.201503329 | 0 |
| Mup13 | 6.29E−165 | −0.875079323 | 6.29E−165 |
| Cyp2c37 | 2.10E−130 | −0.820386118 | 2.10E−130 |
| Cyp2a5 | 1.93E−69 | −0.938183249 | 8.06E−111 |
| Serpina3c | 4.68E−124 | −0.595568461 | 4.68E−124 |
| Adh1 | NA | −0.295980789 | 3.27E−106 |
| Mup8 | 3.80E−123 | −0.829956435 | 3.80E−123 |
| Mup12 | 0 | −1.228078411 | 0 |
| Aldh1a1 | NA | −0.40245097 | 1.39E−102 |
| Adck3 | NA | −0.569692886 | 4.38E−91 |
| Ahcy | 1.57E−115 | −0.49011253 | 1.57E−115 |
| Cox4i1 | 2.23E−168 | −0.57523009 | 2.23E−168 |
| Hsd3b7 | NA | −0.581642142 | 5.15E−93 |
| Mup2 | 5.33E−115 | −0.738721984 | 5.33E−115 |
| Rsp14 | 3.76E−152 | −0.607507888 | 3.76E−152 |
| Ugt2b36 | 4.78E−87 | −0.629671114 | 1.10E−145 |
| Acaa1b | 0 | −0.855527427 | 0 |
| Psen2 | NA | −0.419555606 | 1.69E−77 |
| Cox6c | 2.30E−132 | −0.610222852 | 2.30E−132 |
| Gm4076 | 7.57E−56 | −0.345826319 | 7.98E−74 |
| Uqcrq | 2.42E−124 | −0.485354968 | 2.42E−124 |
| Igfbp2 | 3.60E−190 | −0.717832911 | 3.60E−190 |
| Rnf125 | 8.03E−30 | −0.463320018 | 9.28E−70 |
| mt-Nd6 | 2.74E−110 | −0.685505467 | 2.74E−110 |
| Rps2 | 3.26E−67 | −0.474519664 | 4.24E−69 |
| Hsd3b5 | 4.13E−58 | −0.886463454 | 3.58E−129 |
| Cyp1a2 | 8.09E−18 | −0.481919967 | 3.86E−66 |
| Rpl13a | 2.24E−99 | −0.480174283 | 2.24E−99 |
| Rps27l | 1.42E−139 | −0.64582348 | 1.42E−139 |
| Pon1 | 2.43E−57 | −0.42825679 | 1.97E−64 |
| Gm20594 | 1.33E−34 | −0.573781187 | 4.48E−60 |
| Mvd | NA | −0.327327327 | 2.22E−59 |
| Sparcl1 | 2.01E−42 | −0.253616986 | 3.78E−57 |
| Malat1 | 4.45E−51 | −0.652623643 | 6.64E−56 |
| Cyp8b1 | 1.54E−120 | −0.599290325 | 1.54E−120 |
| Ugt2b34 | 1.12E−106 | −0.500802828 | 1.12E−106 |
| Hacl1 | 2.73E−36 | −0.332899987 | 4.71E−52 |
| Zfp683 | 3.13E−35 | −0.421689112 | 1.10E−47 |
| Oaf | NA | −0.333766768 | 3.11E−47 |
| Cth | 3.95E−48 | −0.339209942 | 3.95E−48 |
| Ndufb11 | 2.10E−70 | −0.479044618 | 2.10E−70 |
| Esd | 5.03E−45 | −0.381539462 | 1.94E−49 |
| Mpc2 | 2.71E−68 | −0.39520967 | 2.71E−68 |
| Ubc | NA | −0.273242853 | 3.58E−41 |
| Hebp1 | 3.96E−26 | −0.313009472 | 1.26E−40 |
| Gm5096 | 9.38E−37 | −0.399548106 | 4.99E−39 |
| Oaz1 | 1.67E−31 | −0.426286153 | 1.27E−51 |
| Cox7b | 3.36E−144 | −0.650085753 | 3.36E−144 |
| Cyp2c55 | 2.21E−52 | −0.347841415 | 2.21E−52 |
| Inmt | 1.56E−139 | −0.679790467 | 1.56E−139 |
| Cyp2e1 | 6.94E−06 | −0.301497571 | 4.23E−36 |
| Clpx | 3.74E−21 | −0.410840842 | 1.11E−53 |
| Ndufb6 | 3.96E−85 | −0.518384931 | 3.96E−85 |
| Hist1h1c | NA | −0.331045753 | 5.78E−33 |
| Elovl3 | 1.57E−229 | −0.924845607 | 1.57E−229 |
| Gm10250 | 1.11E−44 | −0.336847064 | 1.11E−44 |
| Arl4d | NA | −0.328520283 | 5.36E−30 |
| Cml1 | NA | −0.47269345 | 5.55E−63 |
| Hint2 | 3.41E−32 | −0.373773572 | 2.60E−44 |
| Aqp8 | 4.72E−23 | −0.359788885 | 8.19E−45 |
| Ndufa6 | 3.80E−59 | −0.418444742 | 3.80E−59 |
| Gm9843 | 9.49E−46 | −0.396322103 | 9.49E−46 |
| Atp5h | 7.99E−68 | −0.555498653 | 7.99E−68 |
| Dcxr | NA | −0.271339616 | 1.59E−18 |
| Cebpa | NA | −0.481611184 | 2.92E−66 |
| Cyp4a10 | 9.75E−50 | −0.698160085 | 2.61E−98 |
| Sigmar1 | NA | −0.282364617 | 5.67E−17 |
| Nr1i3 | NA | −0.317940692 | 1.11E−16 |
| Ppp2r5a | 3.25E−21 | −0.315098874 | 3.37E−36 |
| Hhex | NA | −0.283483187 | 9.95E−15 |
| Rpl14 | 2.16E−37 | −0.357060958 | 2.16E−37 |
| Tprkb | NA | −0.254084951 | 1.31E−12 |
| Cyp3a59 | NA | −0.263118105 | 3.34E−11 |
| Rps18 | 1.19E−34 | −0.415076007 | 1.19E−34 |
| Ftl1 | 4.66E−11 | −0.347806606 | 4.66E−11 |
| Ubb | NA | −0.263773933 | 0.01387012 |
| Mup1 | 1.78E−91 | −0.882906956 | 1.78E−91 |
| Mup7 | 8.04E−142 | −0.847726732 | 8.04E−142 |
| Gm24245 | NA | 1 | 3.33E−13 |
| Mup14 | 8.52E−68 | 1 | 8.52E−68 |
| Apoa2 | 1.04E−276 | −0.615981195 | 0 |
| Mup20 | 0 | −1.237817166 | 0 |
| Car3 | 0 | −1.292191881 | 0 |
| 2810007J24Rik | 7.72E−74 | −1.093998287 | 2.63E−273 |
| Tdo2 | 2.80E−102 | −0.736504592 | 6.44E−267 |
| Rbp4 | 4.52E−270 | −0.580599925 | 4.52E−270 |
| Fabp1 | 0 | −0.826491833 | 0 |
| Hsd11b1 | 6.95E−53 | −0.998666341 | 3.95E−209 |
| Ces3a | NA | −0.721008165 | 1.41E−206 |
| Hmgcs2 | NA | −0.664269004 | 6.01E−206 |
| Serpina1d | NA | −0.586852756 | 5.86E−199 |
| Selenbp2 | 5.81E−73 | −0.857199402 | 9.70E−194 |
| Hrsp12 | 6.91E−87 | −0.800669662 | 1.58E−182 |
| Ces3b | NA | −0.819558687 | 6.86E−161 |
| Cyp2d9 | 1.56E−76 | −0.616676578 | 8.72E−151 |
| Retsat | 4.31E−216 | −0.847260321 | 4.31E−216 |
| Ugt2b1 | 1.59E−33 | −0.722102286 | 5.69E−140 |
| Serpina3k | 2.79E−242 | −0.298266533 | 2.79E−242 |
| H2-K1 | NA | −0.596584494 | 1.74E−137 |
| Cyp2f2 | NA | −0.887034648 | 5.25E−136 |
| Pigr | NA | −0.661452 | 1.08E−134 |
| Rdh7 | NA | −0.496910675 | 5.58E−132 |
| Apoa1 | 6.60E−253 | −0.411233725 | 6.60E−253 |
| Apoh | NA | −0.417935459 | 3.98E−126 |
| Aadac | 6.31E−69 | −0.747342823 | 6.76E−122 |
| Ephx2 | 1.19E−91 | −0.570800981 | 1.49E−121 |
| Gpx1 | NA | −0.321984135 | 2.36E−120 |
| Alb | NA | −0.427719117 | 2.56E−117 |
| Otc | NA | −0.584972427 | 1.50E−116 |
| Apoa5 | NA | −0.470514722 | 5.32E−149 |
| Mug1 | NA | −0.497189268 | 3.61E−105 |
| Acsl1 | 1.27E−85 | −0.568030382 | 3.63E−104 |
| Cyp4v3 | NA | −0.600614165 | 1.46E−101 |
| Slco1a1 | 6.62E−92 | −0.632477905 | 5.52E−101 |
| Cfhr2 | 1.02E−63 | −0.630617674 | 7.59E−98 |
| B2m | NA | −0.430851295 | 2.11E−97 |
| Khk | NA | −0.530150378 | 8.89E−91 |
| Apoc1 | NA | −0.31073779 | 1.04E−90 |
| Uox | NA | −0.480422343 | 1.42E−89 |
| Rarres2 | NA | −0.492013911 | 1.76E−89 |
| Serpina12 | 1.92E−45 | −0.612367467 | 7.52E−89 |
| Serpinc1 | NA | −0.333556351 | 1.38E−87 |
| Ugt2a3 | 2.95E−72 | −0.54739922 | 2.33E−87 |
| Serpinf1 | NA | −0.506061854 | 3.06E−85 |
| Ttr | NA | −0.286138394 | 3.87E−82 |
| C8g | NA | −0.455638561 | 2.41E−80 |
| Serpina1a | 6.23E−81 | −0.35536875 | 6.23E−81 |
| Serpina1b | NA | −0.294480791 | 1.99E−74 |
| Serpina1e | 0 | −0.72519719 | 0 |
| Etnppl | 1.59E−87 | −0.566620392 | 1.59E−87 |
| 1500017E21Rik | NA | −0.517930222 | 1.93E−69 |
| Atp5f1 | 1.10E−77 | −0.460709866 | 1.10E−77 |
| Ces1f | 9.25E−112 | −0.533638921 | 9.25E−112 |
| Cyp4a12a | NA | −0.4760363 | 1.00E−65 |
| Baat | 1.48E−26 | −0.445833101 | 1.01E−65 |
| Fabp2 | 2.63E−76 | −0.527186942 | 2.63E−76 |
| Serpina1e | NA | −0.279835146 | 1.24E−63 |
| Sod1 | 3.17E−216 | −0.483374172 | 3.17E−216 |
| Cdo1 | NA | −0.343333854 | 2.05E−63 |
| Ech1 | 2.34E−114 | −0.504022226 | 2.34E−114 |
| Insig2 | 2.82E−25 | −0.447606183 | 4.77E−62 |
| Cox6a1 | 4.29E−41 | −0.390617907 | 1.67E−61 |
| Azgp1 | NA | −0.286918536 | 1.16E−60 |
| Ces1c | NA | −0.274525359 | 5.85E−60 |
| Pah | NA | −0.379853874 | 8.65E−60 |
| Lipc | NA | −0.423055461 | 3.19E−59 |
| Dio1 | NA | −0.469732353 | 9.15E−59 |
| Aox3 | NA | −0.429460462 | 1.72E−66 |
| Gstm1 | 1.73E−60 | −0.448355325 | 1.73E−60 |
| Slc27a5 | NA | −0.374625102 | 1.08E−57 |
| Amy1 | NA | −0.441174 | 1.40E−57 |
| Tmem205 | NA | −0.363762607 | 6.01E−57 |
| Cyp2a12 | NA | −0.396631721 | 6.96E−57 |
| Angptl3 | 2.66E−169 | −0.529458826 | 2.66E−169 |
| Cyp7b1 | NA | −0.438158056 | 1.37E−54 |
| Hpd | NA | −0.317943335 | 2.00E−54 |
| Ugp2 | NA | −0.394209561 | 6.59E−54 |
| Ndufa4 | 4.13E−107 | −0.512722876 | 4.13E−107 |
| Nudt7 | NA | −0.566043759 | 9.48E−52 |
| Slc10a1 | 4.19E−30 | −0.395488081 | 8.15E−51 |
| Scp2 | NA | −0.319519472 | 5.16E−159 |
| Sco2 | 3.07E−16 | −0.395951512 | 1.04E−49 |
| Cyp2d10 | 9.23E−44 | −0.325332653 | 3.76E−49 |
| Chchd2 | 1.10E−119 | −0.518624159 | 1.10E−119 |
| Idh3b | NA | −0.360516268 | 4.19E−48 |
| Bckdha | NA | −0.404702898 | 2.27E−47 |
| Glo1 | 1.59E−34 | −0.379233492 | 3.51E−47 |
| Sdhb | NA | −0.357362255 | 2.51E−46 |
| Cat | NA | −0.286016441 | 1.00E−45 |
| Ces1e | 9.70E−44 | −0.375733204 | 2.06E−45 |
| Cox5b | 5.23E−43 | −0.393691854 | 3.39E−45 |
| Atp5g3 | 1.87E−78 | −0.357248453 | 1.87E−78 |
| Itm2b | NA | −0.261048932 | 4.20E−44 |
| Etfdh | NA | −0.33708519 | 1.81E−43 |
| Gsto1 | NA | −0.368935961 | 5.15E−43 |
| Hsd3b3 | NA | −0.347460551 | 3.82E−42 |
| Slc27a2 | NA | −0.305294922 | 2.23E−41 |
| Hsd17b6 | 9.45E−46 | −0.364685233 | 9.45E−46 |
| Abcd3 | NA | −0.326838469 | 1.45E−40 |
| Mdh1 | NA | −0.329025797 | 2.04E−40 |
| Prdx4 | 1.74E−28 | −0.378024564 | 3.39E−40 |
| Hsd17b13 | NA | −0.376251609 | 1.14E−64 |
| Proc | NA | −0.319776879 | 1.66E−39 |
| Agxt | NA | −0.303827932 | 1.79E−39 |
| Gls2 | NA | −0.353616749 | 1.09E−57 |
| Ugt2b38 | NA | −0.250921219 | 1.20E−38 |
| Ndufb9 | 1.07E−104 | −0.483703094 | 1.07E−104 |
| Rabac1 | 1.41E−19 | −0.342063679 | 3.25E−38 |
| Slc22a30 | 2.47E−15 | −0.331870745 | 7.77E−38 |
| Etfa | NA | −0.33793403 | 8.07E−38 |
| Atp5j | 7.16E−66 | −0.415380683 | 7.16E−66 |
| Uqcrfs1 | NA | −0.335765197 | 5.60E−37 |
| Cyp27a1 | NA | −0.326228094 | 1.46E−36 |
| Hal | NA | −0.304647432 | 3.02E−36 |
| Sar1b | 6.58E−68 | −0.422088437 | 6.58E−68 |
| Aldh6a1 | NA | −0.30870904 | 4.64E−36 |
| Il1rap | NA | −0.302807638 | 1.94E−35 |
| S100a1 | NA | −0.355544479 | 2.21E−35 |
| Creg1 | 1.51E−125 | −0.480113825 | 1.51E−125 |
| 1300017J02Rik | NA | −0.308343818 | 1.20E−34 |
| Acat1 | NA | −0.290805818 | 1.81E−34 |
| Pklr | NA | −0.283149611 | 4.04E−64 |
| Dpys | NA | −0.313405671 | 8.84E−34 |
| Aadat | NA | −0.325731706 | 9.13E−34 |
| Cd81 | NA | −0.30883572 | 1.50E−33 |
| Ndufc2 | 9.91E−31 | −0.371342649 | 1.56E−33 |
| Bphl | NA | −0.299478155 | 1.70E−33 |
| Qdpr | NA | −0.316595182 | 1.80E−33 |
| Gsta2 | NA | −0.284180552 | 4.56E−33 |
| Acsm3 | NA | −0.280708005 | 5.27E−33 |
| Cyp2d26 | NA | −0.270278674 | 2.04E−32 |
| Cyp2c44 | NA | −0.312764543 | 2.09E−32 |
| Hagh | 4.45E−73 | −0.398106396 | 4.45E−73 |
| Ces2a | 7.84E−141 | −0.619093582 | 7.84E−141 |
| Gas5 | NA | −0.298417518 | 6.31E−32 |
| Uqcrb | 6.07E−33 | −0.359656695 | 6.07E−33 |
| Gm4788 | NA | −0.297042332 | 1.49E−31 |
| Slc2a2 | NA | −0.291267847 | 2.49E−31 |
| Cela1 | NA | −0.252611197 | 1.03E−30 |
| Hgd | NA | −0.273998205 | 2.46E−30 |
| 1110008F13Rik | 1.42E−34 | −0.327482854 | 1.42E−34 |
| Csad | 5.27E−267 | −1.214863323 | 5.27E−267 |
| Gabarapl1 | NA | −0.297156374 | 2.02E−29 |
| Eci1 | 3.71E−30 | −0.31335345 | 3.71E−30 |
| Amdhd1 | NA | −0.272469871 | 8.10E−29 |
| Uqcrh | 5.89E−83 | −0.483113531 | 5.89E−83 |
| 0610011F06Rik | NA | −0.28593883 | 3.51E−28 |
| Ftcd | NA | −0.299187456 | 5.47E−28 |
| Cyp4f14 | NA | −0.283565035 | 6.47E−28 |
| Ttc39c | NA | −0.289612311 | 2.34E−27 |
| Ndufv1 | NA | −0.256865664 | 2.43E−27 |
| Pdhb | NA | −0.271619421 | 5.48E−27 |
| Hsd17b11 | NA | −0.272884574 | 8.36E−27 |
| Fcgrt | NA | −0.270945405 | 1.81E−26 |
| Lyrm5 | NA | −0.251171881 | 3.08E−26 |
| Bnip3 | 1.43E−18 | −0.275946132 | 3.10E−26 |
| Pygl | NA | −0.25405228 | 1.29E−25 |
| C8b | NA | −0.251084368 | 1.50E−25 |
| Atp5j2 | 3.23E−66 | −0.409576712 | 3.23E−66 |
| Fxyd1 | NA | −0.273422293 | 2.95E−25 |
| Nit2 | NA | −0.255321274 | 6.79E−25 |
| Sdha | NA | −0.286156431 | 7.96E−25 |
| Pccb | NA | −0.266663529 | 8.00E−25 |
| Park7 | NA | −0.270929309 | 9.02E−25 |
| Cmbl | NA | −0.265365994 | 9.66E−25 |
| Ces2e | NA | −0.272708804 | 8.71E−24 |
| Cebpb | 3.07E−10 | −0.306330499 | 1.73E−23 |
| Pbld1 | NA | −0.264627734 | 2.23E−23 |
| Adipor2 | NA | −0.252551165 | 1.11E−22 |
| Ephx1 | 2.60E−38 | −0.332038055 | 2.60E−38 |
| C6 | NA | −0.255102014 | 2.04E−21 |
| Rn7sk | NA | −0.276399379 | 6.19E−14 |
| Hint1 | 9.22E−101 | −0.487016283 | 9.22E−101 |
| Gstp1 | 4.28E−90 | −0.671664785 | 4.28E−90 |
| Gnmt | 8.65E−84 | −0.288175489 | 8.65E−84 |
| Sc4mol | 1.06E−80 | −0.519568919 | 1.06E−80 |
| Prdx1 | 2.27E−73 | −0.353764247 | 2.27E−73 |
| Rpl32 | 4.42E−73 | −0.498592915 | 4.42E−73 |
| Uqcr11 | 8.58E−61 | −0.368070557 | 8.58E−61 |
| Cox7a2 | 3.27E−59 | −0.441101884 | 3.27E−59 |
| Rps3 | 7.09E−57 | −0.339903958 | 7.09E−57 |
| Ndufb8 | 1.46E−55 | −0.389897205 | 1.46E−55 |
| Etfb | 4.81E−54 | −0.270014172 | 4.81E−54 |
| Leap2 | 1.27E−50 | −0.404792721 | 1.27E−50 |
| Uqcr10 | 1.93E−47 | −0.382321788 | 1.93E−47 |
| Akr1c19 | 1.32E−46 | −0.38253326 | 1.32E−46 |
| Ddt | 1.40E−46 | −0.345838673 | 1.40E−46 |
| Eef1b2 | 1.90E−45 | −0.367328377 | 1.90E−45 |
| Chchd10 | 5.22E−43 | −0.28090926 | 5.22E−43 |
| Rps24 | 6.91E−41 | −0.386464837 | 6.91E−41 |
| Acot1 | 2.65E−38 | −0.299327096 | 2.65E−38 |
| Cyp3a44 | 2.41E−34 | −0.297417766 | 2.41E−34 |
| Ndufb4 | 2.14E−33 | −0.331687642 | 2.14E−33 |
| Sdhd | 2.47E−32 | −0.302302961 | 2.47E−32 |
| Rps5 | 1.17E−30 | −0.25674004 | 1.17E−30 |
| Ndufb2 | 1.21E−30 | −0.336855533 | 1.21E−30 |
| Blvrb | 2.20E−30 | −0.351075319 | 2.20E−30 |
| Ndufs6 | 2.06E−29 | −0.341035035 | 2.06E−29 |
| Cnbp | 2.46E−29 | −0.286746587 | 2.46E−29 |
| Cyp4a14 | 1.10E−28 | −0.31778609 | 1.10E−28 |
| Txn1 | 6.45E−28 | −0.277353299 | 6.45E−28 |
| Rpl36al | 3.44E−26 | −0.331844566 | 3.44E−26 |
| Gadd45b | 4.38E−25 | −0.301158726 | 4.38E−25 |
| G0s2 | 5.23E−25 | −0.269113384 | 5.23E−25 |
| Ssr4 | 7.71E−23 | −0.283161199 | 7.71E−23 |
| Minos1 | 8.00E−23 | −0.308403636 | 8.00E−23 |
| Ndufa2 | 9.67E−23 | −0.308306345 | 9.67E−23 |
| Tmem243 | 2.06E−22 | −0.275109242 | 2.06E−22 |
| Rps21 | 2.81E−22 | −0.316866281 | 2.81E−22 |
| Ndufa5 | 4.19E−22 | −0.310280044 | 4.19E−22 |
| Tubb2a | 5.24E−22 | −0.260491764 | 5.24E−22 |
| Akr1c14 | 1.20E−21 | −0.280169437 | 1.20E−21 |
| Atp5o | 2.25E−21 | −0.268645357 | 2.25E−21 |
| Psmb1 | 5.68E−21 | −0.271574562 | 5.68E−21 |
| Rps9 | 6.38E−21 | −0.30861002 | 6.38E−21 |
| Nme1 | 9.48E−21 | −0.291609359 | 9.48E−21 |
| Ndufb5 | 1.33E−20 | −0.274484363 | 1.33E−20 |
| Psma6 | 2.82E−20 | −0.281709731 | 2.82E−20 |
| Rplp1 | 6.89E−20 | −0.274821325 | 6.89E−20 |
| Fam162a | 1.14E−19 | −0.280519821 | 1.14E−19 |
| 4931406C07Rik | 3.10E−19 | −0.28178071 | 3.10E−19 |
| Sc5d | 3.96E−19 | −0.274286355 | 3.96E−19 |
| Timm13 | 1.44E−18 | −0.266267617 | 1.44E−18 |
| Ndufs4 | 2.26E−18 | −0.299960018 | 2.26E−18 |
| Selk | 4.58E−18 | −0.279248941 | 4.58E−18 |
| Mrps18c | 1.08E−17 | −0.27427115 | 1.08E−17 |
| Rpl13 | 3.52E−17 | −0.314606263 | 7.45E−64 |
| Rps8 | 2.52E−16 | −0.279845708 | 2.52E−16 |
| Rps15 | 3.43E−16 | −0.324542895 | 3.43E−16 |
| N1rp12 | 1.08E−15 | −0.269307598 | 1.08E−15 |
| Timm8b | 2.42E−15 | −0.25098905 | 2.42E−15 |
| Dhrs4 | 5.96E−15 | −0.268862728 | 5.96E−15 |
| Rps10 | 3.85E−13 | −0.252659935 | 3.85E−13 |
| Rpl34 | 1.43E−12 | −0.264586248 | 1.43E−12 |
| Hspe1 | 3.13E−12 | −0.257910168 | 3.13E−12 |
| Usmg5 | 1.10E−10 | −0.28365494 | 1.10E−10 |
| Rpl26 | 1.36E−09 | −0.264487941 | 1.36E−09 |
| Rps19 | 5.15E−06 | −0.25849266 | 5.15E−06 |

TABLE 8C
Differentially expressed genes between treatment (APAP and PH).

| | p_val | avg_logFC | pct.1 | pct.2 | p_val_adj | cluster | gene |
|---|---|---|---|---|---|---|---|
| Hbb-bs | 0 | 2.585900747 | 0.537 | 0.038 | 0 | A6 | Hbb-bs |
| S100a9 | 0 | 1.342310796 | 0.399 | 0.005 | 0 | A6 | S100a9 |
| Hmox1 | 0 | 1.339570861 | 0.558 | 0.026 | 0 | A6 | Hmox1 |
| S100a8 | 0 | 1.127181553 | 0.353 | 0.002 | 0 | A6 | S100a8 |
| Tnfrsf12a | 4.64E−274 | 1.104985626 | 0.623 | 0.086 | 1.15E−269 | A6 | Tnfrsf12a |
| Ccl6 | 8.63E−274 | 0.786807473 | 0.274 | 0.006 | 2.14E−269 | A6 | Ccl6 |
| Hilpda | 3.58E−254 | 0.780347895 | 0.403 | 0.028 | 8.90E−250 | A6 | Hilpda |
| Mt2 | 4.30E−249 | 2.037681937 | 0.938 | 0.405 | 1.07E−244 | A6 | Mt2 |
| Srgn | 3.95E−244 | 0.928927613 | 0.284 | 0.01 | 9.80E−240 | A6 | Srgn |
| Mt1 | 5.85E−244 | 2.320387581 | 0.981 | 0.602 | 1.45E−239 | A6 | Mt1 |
| Srxn1 | 1.13E−243 | 1.864212779 | 0.821 | 0.239 | 2.80E−239 | A6 | Srxn1 |
| Ppl | 1.02E−233 | 0.861750145 | 0.47 | 0.049 | 2.54E−229 | A6 | Ppl |
| Slc16a6 | 7.70E−208 | 0.913971966 | 0.47 | 0.058 | 1.91E−203 | A6 | Slc16a6 |
| Gclc | 1.87E−200 | 1.802382727 | 0.962 | 0.764 | 4.64E−196 | A6 | Gclc |
| Cdkn1a | 1.22E−180 | 1.251876494 | 0.702 | 0.195 | 3.04E−176 | A6 | Cdkn1a |
| Plin2 | 1.48E−175 | 1.454290064 | 0.943 | 0.742 | 3.67E−171 | A6 | Plin2 |
| Tubb6 | 1.52E−170 | 0.571531819 | 0.368 | 0.04 | 3.78E−166 | A6 | Tubb6 |
| Actb | 9.38E−169 | 1.051604326 | 0.979 | 0.893 | 2.33E−164 | A6 | Actb |
| Clec4f | 3.46E−163 | 1.124057361 | 0.339 | 0.034 | 8.59E−159 | A6 | Clec4f |
| Chka | 5.96E−156 | 1.403236367 | 0.716 | 0.24 | 1.48E−151 | A6 | Chka |
| Txnrd1 | 7.87E−143 | 1.374708706 | 0.838 | 0.516 | 1.95E−138 | A6 | Txnrd1 |
| Il1r1 | 5.63E−137 | 1.188669997 | 0.683 | 0.247 | 1.40E−132 | A6 | Il1r1 |
| Krt18 | 3.50E−128 | 1.22726005 | 0.864 | 0.653 | 8.70E−124 | A6 | Krt18 |
| Cidec | 1.77E−123 | 0.30673991 | 0.253 | 0.024 | 4.40E−119 | A6 | Cidec |
| Krt8 | 8.28E−122 | 1.273475842 | 0.826 | 0.564 | 2.06E−117 | A6 | Krt8 |
| Klf6 | 8.56E−120 | 0.70668227 | 0.382 | 0.066 | 2.13E−115 | A6 | Klf6 |
| Ets2 | 2.20E−109 | 0.68055198 | 0.415 | 0.087 | 5.47E−105 | A6 | Ets2 |
| 2010003K11Rik | 3.63E−105 | 0.572900848 | 0.396 | 0.081 | 9.02E−101 | A6 | 2010003K11Rik |
| Tmsb4x | 6.61E−105 | 0.791101995 | 0.332 | 0.055 | 1.64E−100 | A6 | Tmsb4x |
| Cers6 | 1.33E−104 | 0.50707625 | 0.332 | 0.055 | 3.29E−100 | A6 | Cers6 |
| Plscr1 | 2.32E−103 | 0.449697464 | 0.329 | 0.055 | 5.75E−99 | A6 | Plscr1 |
| Rcan1 | 1.90E−94 | 0.700981932 | 0.425 | 0.105 | 4.72E−90 | A6 | Rcan1 |
| Gm26924 | 2.05E−93 | 0.726922776 | 1 | 1 | 5.10E−89 | A6 | Gm26924 |
| Myh9 | 2.39E−90 | 1.024834729 | 0.718 | 0.401 | 5.92E−86 | A6 | Myh9 |
| Fgg | 1.41E−82 | 0.47507836 | 0.986 | 0.995 | 3.50E−78 | A6 | Fgg |
| Serpina3m | 2.54E−82 | 0.793187331 | 0.85 | 0.772 | 6.30E−78 | A6 | Serpina3m |
| C230081A13Rik | 5.32E−82 | 0.418417199 | 0.279 | 0.049 | 1.32E−77 | A6 | C230081A13Rik |
| Lrrc58 | 6.20E−82 | 0.939637116 | 0.711 | 0.429 | 1.54E−77 | A6 | Lrrc58 |
| Fga | 8.43E−81 | 0.50099721 | 0.986 | 0.997 | 2.09E−76 | A6 | Fga |
| Apoa4 | 3.02E−80 | 1.166038714 | 0.721 | 0.434 | 7.51E−76 | A6 | Apoa4 |
| Rell1 | 5.69E−79 | 0.579256897 | 0.375 | 0.095 | 1.41E−74 | A6 | Rell1 |
| Bcl2l1 | 9.17E−79 | 0.741502458 | 0.501 | 0.182 | 2.28E−74 | A6 | Bcl2l1 |
| Fgb | 2.77E−76 | 0.477409638 | 0.995 | 1 | 6.88E−72 | A6 | Fgb |
| Lars2 | 4.16E−75 | 0.814259119 | 1 | 0.995 | 1.03E−70 | A6 | Lars2 |
| D10Wsu102e | 3.45E−74 | 0.569827642 | 0.37 | 0.097 | 8.56E−70 | A6 | D10Wsu102e |
| Gsta1 | 2.23E−70 | 0.408922304 | 0.32 | 0.073 | 5.53E−66 | A6 | Gsta1 |
| Slc39a14 | 6.11E−70 | 0.916094428 | 0.678 | 0.428 | 1.52E−65 | A6 | Slc39a14 |
| Mrap | 9.06E−70 | 0.861482917 | 0.606 | 0.323 | 2.25E−65 | A6 | Mrap |
| Gm15564 | 3.80E−66 | 0.786113662 | 0.983 | 0.967 | 9.43E−62 | A6 | Gm15564 |
| 1700017B05Rik | 1.21E−62 | 0.327661725 | 0.255 | 0.052 | 3.00E−58 | A6 | 1700017B05Rik |
| Igfbp1 | 9.02E−61 | 1.089898094 | 0.792 | 0.702 | 2.24E−56 | A6 | Igfbp1 |
| Sdc4 | 7.44E−59 | 0.567251857 | 0.909 | 0.892 | 1.85E−54 | A6 | Sdc4 |
| Cldn14 | 1.21E−58 | 0.369504106 | 0.32 | 0.085 | 3.00E−54 | A6 | Cldn14 |
| Alb | 4.24E−58 | 0.374865074 | 1 | 1 | 1.05E−53 | A6 | Alb |
| Sat1 | 3.50E−57 | 0.688728161 | 0.47 | 0.193 | 8.68E−53 | A6 | Sat1 |
| Hnf4a | 7.04E−57 | 0.696248102 | 0.835 | 0.805 | 1.75E−52 | A6 | Hnf4a |
| Sqstm1 | 1.54E−56 | 0.699213054 | 0.859 | 0.828 | 3.83E−52 | A6 | Sqstm1 |
| Fam134b | 3.22E−56 | 0.815231577 | 0.678 | 0.482 | 8.00E−52 | A6 | Fam134b |
| Nop58 | 2.76E−54 | 0.634296193 | 0.492 | 0.216 | 6.86E−50 | A6 | Nop58 |
| Efhd2 | 4.11E−53 | 0.576644145 | 0.468 | 0.197 | 1.02E−48 | A6 | Efhd2 |
| mmu-mir-6240 | 5.36E−53 | 1.031610172 | 0.413 | 0.149 | 1.33E−48 | A6 | mmu-mir-6240 |
| Trp53inp1 | 9.22E−52 | 0.882913173 | 0.637 | 0.398 | 2.29E−47 | A6 | Trp53inp1 |
| Epas1 | 5.89E−51 | 0.713771221 | 0.604 | 0.373 | 1.46E−46 | A6 | Epas1 |
| Apob | 8.74E−50 | 0.480248058 | 0.99 | 0.989 | 2.17E−45 | A6 | Apob |
| Gcnt2 | 4.90E−49 | 0.503845296 | 0.372 | 0.131 | 1.22E−44 | A6 | Gcnt2 |
| Eif1a | 5.09E−49 | 0.722796481 | 0.599 | 0.373 | 1.26E−44 | A6 | Eif1a |
| Insig2 | 1.13E−47 | 0.608089025 | 0.854 | 0.83 | 2.80E−43 | A6 | Insig2 |
| Serpina3n | 2.16E−47 | 0.664148633 | 0.747 | 0.658 | 5.38E−43 | A6 | Serpina3n |
| Btg2 | 1.37E−46 | 0.766889798 | 0.549 | 0.303 | 3.40E−42 | A6 | Btg2 |
| Agpat9 | 3.40E−46 | 0.304853757 | 0.277 | 0.077 | 8.45E−42 | A6 | Agpat9 |
| Tra2a | 1.23E−45 | 0.708051809 | 0.337 | 0.114 | 3.05E−41 | A6 | Tra2a |
| Creb3l2 | 1.45E−44 | 0.397122997 | 0.329 | 0.11 | 3.61E−40 | A6 | Creb3l2 |
| Stat3 | 1.26E−43 | 0.651998495 | 0.604 | 0.387 | 3.12E−39 | A6 | Stat3 |
| Picalm | 3.98E−43 | 0.676555334 | 0.647 | 0.452 | 9.89E−39 | A6 | Picalm |
| Tubb2a | 8.70E−43 | 0.785991182 | 0.618 | 0.443 | 2.16E−38 | A6 | Tubb2a |
| Mat1a | 9.42E−43 | 0.388871208 | 0.986 | 0.973 | 2.34E−38 | A6 | Mat1a |
| Malat1 | 3.57E−42 | 0.985365756 | 0.871 | 0.75 | 8.87E−38 | A6 | Malat1 |
| Cxadr | 1.77E−41 | 0.656840533 | 0.606 | 0.408 | 4.40E−37 | A6 | Cxadr |
| Myo1e | 5.97E−41 | 0.578639718 | 0.475 | 0.237 | 1.48E−36 | A6 | Myo1e |
| Bach1 | 8.93E−41 | 0.76858139 | 0.461 | 0.236 | 2.22E−36 | A6 | Bach1 |
| Eif5 | 1.03E−40 | 0.480890827 | 0.816 | 0.786 | 2.56E−36 | A6 | Eif5 |
| Tiparp | 2.49E−40 | 0.524808466 | 0.346 | 0.131 | 6.18E−36 | A6 | Tiparp |
| Cltc | 7.70E−40 | 0.685231275 | 0.661 | 0.494 | 1.91E−35 | A6 | Cltc |
| Ddx21 | 1.55E−39 | 0.655929312 | 0.535 | 0.316 | 3.84E−35 | A6 | Ddx21 |
| Mdn1 | 2.43E−39 | 0.30793324 | 0.251 | 0.072 | 6.04E−35 | A6 | Mdn1 |
| Heatr1 | 4.55E−39 | 0.381462092 | 0.291 | 0.095 | 1.13E−34 | A6 | Heatr1 |
| H3f3b | 4.97E−39 | 0.718378155 | 0.613 | 0.443 | 1.23E−34 | A6 | H3f3b |
| Jmjd1c | 3.17E−38 | 0.596320002 | 0.47 | 0.239 | 7.87E−34 | A6 | Jmjd1c |
| Dot1l | 4.16E−38 | 0.38526892 | 0.282 | 0.092 | 1.03E−33 | A6 | Dot1l |
| Ctsl | 6.53E−38 | 0.51275344 | 0.878 | 0.88 | 1.62E−33 | A6 | Ctsl |
| Slc10a2 | 1.11E−37 | 0.270447751 | 0.255 | 0.077 | 2.75E−33 | A6 | Slc10a2 |
| B4galt5 | 1.26E−37 | 0.423884611 | 0.332 | 0.126 | 3.12E−33 | A6 | B4galt5 |
| Rock2 | 2.45E−37 | 0.555177626 | 0.425 | 0.199 | 6.08E−33 | A6 | Rock2 |
| Dgkd | 6.05E−37 | 0.358416905 | 0.282 | 0.094 | 1.50E−32 | A6 | Dgkd |
| Lcn2 | 1.67E−36 | 0.387574636 | 0.301 | 0.106 | 4.14E−32 | A6 | Lcn2 |
| Hspb8 | 1.95E−36 | 0.608924075 | 0.709 | 0.633 | 4.84E−32 | A6 | Hspb8 |
| Ugdh | 3.59E−36 | 0.608545157 | 0.721 | 0.647 | 8.92E−32 | A6 | Ugdh |
| Ppan | 9.70E−36 | 0.276685555 | 0.255 | 0.079 | 2.41E−31 | A6 | Ppan |
| Xbp1 | 1.86E−35 | 0.569393474 | 0.692 | 0.611 | 4.62E−31 | A6 | Xbp1 |
| Ddx5 | 2.16E−35 | 0.539662714 | 0.685 | 0.543 | 5.37E−31 | A6 | Ddx5 |
| Tnfrsf1a | 2.76E−35 | 0.568140336 | 0.508 | 0.305 | 6.86E−31 | A6 | Tnfrsf1a |
| Tsr1 | 5.76E−35 | 0.339217996 | 0.341 | 0.135 | 1.43E−30 | A6 | Tsr1 |
| Itih3 | 6.08E−35 | 0.404217046 | 0.907 | 0.913 | 1.51E−30 | A6 | Itih3 |
| Ddx3x | 6.10E−35 | 0.561165624 | 0.692 | 0.592 | 1.51E−30 | A6 | Ddx3x |
| St3gal1 | 9.22E−35 | 0.516994152 | 0.473 | 0.262 | 2.29E−30 | A6 | St3gal1 |
| Eaf1 | 1.20E−34 | 0.50717323 | 0.427 | 0.212 | 2.98E−30 | A6 | Eaf1 |
| Hsd17b13 | 2.16E−34 | 0.480378763 | 0.864 | 0.761 | 5.37E−30 | A6 | Hsd17b13 |
| Abca1 | 1.44E−33 | 0.65392515 | 0.604 | 0.438 | 3.58E−29 | A6 | Abca1 |
| Hif1a | 4.80E−33 | 0.487565104 | 0.427 | 0.213 | 1.19E−28 | A6 | Hif1a |
| Tmem87b | 1.08E−32 | 0.405411811 | 0.351 | 0.151 | 2.68E−28 | A6 | Tmem87b |
| Fkbp5 | 1.20E−32 | 0.456737162 | 0.415 | 0.197 | 2.97E−28 | A6 | Fkbp5 |
| C3 | 2.78E−32 | 0.278458208 | 0.993 | 0.993 | 6.90E−28 | A6 | C3 |
| Spred1 | 3.89E−31 | 0.352013469 | 0.308 | 0.122 | 9.65E−27 | A6 | Spred1 |
| Eef2 | 8.96E−31 | 0.405940418 | 0.9 | 0.899 | 2.23E−26 | A6 | Eef2 |
| Gnl3 | 1.03E−30 | 0.49017644 | 0.501 | 0.3 | 2.56E−26 | A6 | Gnl3 |
| Itih2 | 1.23E−30 | 0.392672893 | 0.893 | 0.914 | 3.06E−26 | A6 | Itih2 |
| Stk40 | 4.64E−30 | 0.493002444 | 0.382 | 0.184 | 1.15E−25 | A6 | Stk40 |
| Tjp2 | 4.42E−29 | 0.396131523 | 0.332 | 0.147 | 1.10E−24 | A6 | Tjp2 |
| Lyst | 1.82E−28 | 0.371733248 | 0.31 | 0.127 | 4.52E−24 | A6 | Lyst |
| Errfi1 | 2.21E−28 | 0.342085301 | 0.943 | 0.931 | 5.50E−24 | A6 | Errfi1 |
| Chic2 | 3.18E−28 | 0.396396418 | 0.37 | 0.181 | 7.89E−24 | A6 | Chic2 |
| Rad54l2 | 3.33E−28 | 0.364207232 | 0.325 | 0.139 | 8.26E−24 | A6 | Rad54l2 |
| Mfsd2a | 4.59E−28 | 0.461048631 | 0.465 | 0.263 | 1.14E−23 | A6 | Mfsd2a |
| Sdad1 | 1.02E−27 | 0.296939107 | 0.289 | 0.115 | 2.54E−23 | A6 | Sdad1 |
| Bcl3 | 2.52E−27 | 0.274060002 | 0.277 | 0.109 | 6.26E−23 | A6 | Bcl3 |
| Neat1 | 3.08E−27 | 0.655410081 | 0.31 | 0.135 | 7.66E−23 | A6 | Neat1 |
| Asl | 4.46E−27 | 0.41345396 | 0.826 | 0.822 | 1.11E−22 | A6 | Asl |
| Tcerg1 | 5.22E−27 | 0.346807528 | 0.305 | 0.132 | 1.30E−22 | A6 | Tcerg1 |
| Tacc2 | 5.92E−27 | 0.312763874 | 0.289 | 0.116 | 1.47E−22 | A6 | Tacc2 |
| Gpt2 | 9.88E−27 | 0.469474425 | 0.764 | 0.767 | 2.45E−22 | A6 | Gpt2 |
| Abhd2 | 2.12E−26 | 0.414892908 | 0.37 | 0.181 | 5.25E−22 | A6 | Abhd2 |
| Cyp4a14 | 2.69E−26 | 0.333371288 | 0.776 | 0.554 | 6.69E−22 | A6 | Cyp4a14 |
| Ptpn1 | 4.30E−26 | 0.386530538 | 0.353 | 0.165 | 1.07E−21 | A6 | Ptpn1 |
| Fndc3b | 9.70E−26 | 0.349610838 | 0.315 | 0.14 | 2.41E−21 | A6 | Fndc3b |
| Tsku | 3.63E−25 | 0.333177586 | 0.327 | 0.148 | 9.01E−21 | A6 | Tsku |
| Nolc1 | 6.52E−25 | 0.444492735 | 0.363 | 0.183 | 1.62E−20 | A6 | Nolc1 |
| Jak1 | 1.07E−24 | 0.543711884 | 0.554 | 0.415 | 2.67E−20 | A6 | Jak1 |
| Tsc22d2 | 2.31E−24 | 0.362913692 | 0.353 | 0.175 | 5.73E−20 | A6 | Tsc22d2 |
| Btg1 | 2.53E−24 | 0.398422802 | 0.356 | 0.177 | 6.29E−20 | A6 | Btg1 |
| Son | 2.57E−24 | 0.455496144 | 0.601 | 0.439 | 6.39E−20 | A6 | Son |
| Lpin2 | 3.86E−24 | 0.516521417 | 0.745 | 0.687 | 9.59E−20 | A6 | Lpin2 |
| Iigp1 | 5.96E−24 | 0.643134023 | 0.78 | 0.805 | 1.48E−19 | A6 | Iigp1 |
| St3gal5 | 6.19E−24 | 0.435791231 | 0.706 | 0.581 | 1.54E−19 | A6 | St3gal5 |
| n-R5-8s1 | 1.02E−23 | 0.400510777 | 0.308 | 0.135 | 2.53E−19 | A6 | n-R5-8s1 |
| Eif4g2 | 1.15E−23 | 0.412563051 | 0.757 | 0.748 | 2.86E−19 | A6 | Eif4g2 |
| Gjb2 | 2.31E−23 | 0.503822785 | 0.678 | 0.644 | 5.73E−19 | A6 | Gjb2 |
| Dusp16 | 1.08E−22 | 0.427321245 | 0.372 | 0.203 | 2.68E−18 | A6 | Dusp16 |
| Pfkfb2 | 1.30E−22 | 0.269394194 | 0.26 | 0.108 | 3.23E−18 | A6 | Pfkfb2 |
| Tars | 1.56E−22 | 0.471782286 | 0.666 | 0.587 | 3.87E−18 | A6 | Tars |
| Rora | 2.50E−22 | 0.540962509 | 0.549 | 0.433 | 6.22E−18 | A6 | Rora |
| Plec | 3.98E−22 | 0.418829547 | 0.422 | 0.244 | 9.88E−18 | A6 | Plec |
| Gm26917 | 6.93E−22 | 0.423906459 | 0.382 | 0.204 | 1.72E−17 | A6 | Gm26917 |
| Mpeg1 | 1.75E−21 | 0.466789938 | 0.263 | 0.113 | 4.34E−17 | A6 | Mpeg1 |
| Mat2a | 3.17E−21 | 0.475830508 | 0.487 | 0.332 | 7.86E−17 | A6 | Mat2a |
| Gtpbp4 | 3.37E−21 | 0.462339077 | 0.477 | 0.317 | 8.37E−17 | A6 | Gtpbp4 |
| Ranbp2 | 4.52E−21 | 0.507425104 | 0.566 | 0.435 | 1.12E−16 | A6 | Ranbp2 |
| Map2k3 | 6.02E−21 | 0.414970013 | 0.427 | 0.262 | 1.50E−16 | A6 | Map2k3 |
| Mcl1 | 6.44E−21 | 0.524900127 | 0.573 | 0.434 | 1.60E−16 | A6 | Mcl1 |
| Slc20a1 | 1.39E−20 | 0.326037889 | 0.284 | 0.131 | 3.44E−16 | A6 | Slc20a1 |
| Cars | 1.86E−20 | 0.306735271 | 0.329 | 0.166 | 4.61E−16 | A6 | Cars |
| Ywhag | 3.03E−20 | 0.513182681 | 0.518 | 0.392 | 7.52E−16 | A6 | Ywhag |
| Retsat | 4.73E−20 | 0.291831196 | 0.845 | 0.85 | 1.18E−15 | A6 | Retsat |
| Etf1 | 4.99E−20 | 0.452556434 | 0.606 | 0.499 | 1.24E−15 | A6 | Etf1 |
| Epb4.1 | 6.09E−20 | 0.467770072 | 0.537 | 0.403 | 1.51E−15 | A6 | Epb4.1 |
| Myl12a | 8.41E−20 | 0.402881373 | 0.461 | 0.306 | 2.09E−15 | A6 | Myl12a |
| Dusp6 | 3.85E−19 | 0.508677973 | 0.482 | 0.335 | 9.57E−15 | A6 | Dusp6 |
| Gm20594 | 7.21E−19 | 0.440502144 | 0.442 | 0.259 | 1.79E−14 | A6 | Gm20594 |
| Lrg1 | 7.52E−19 | 0.332404269 | 0.783 | 0.797 | 1.87E−14 | A6 | Lrg1 |
| Dkc1 | 8.07E−19 | 0.30544641 | 0.315 | 0.162 | 2.00E−14 | A6 | Dkc1 |
| App | 1.30E−18 | 0.338059459 | 0.315 | 0.162 | 3.23E−14 | A6 | App |
| Rnd3 | 1.41E−18 | 0.326746608 | 0.284 | 0.14 | 3.50E−14 | A6 | Rnd3 |
| Ncl | 1.56E−18 | 0.388188482 | 0.69 | 0.639 | 3.88E−14 | A6 | Ncl |
| Tatdn2 | 1.76E−18 | 0.324760981 | 0.334 | 0.176 | 4.36E−14 | A6 | Tatdn2 |
| Fam107b | 2.12E−18 | 0.416215114 | 0.449 | 0.307 | 5.26E−14 | A6 | Fam107b |
| Tns1 | 3.23E−18 | 0.400122808 | 0.403 | 0.254 | 8.02E−14 | A6 | Tns1 |
| Phc2 | 3.78E−18 | 0.298986746 | 0.284 | 0.141 | 9.39E−14 | A6 | Phc2 |
| Tbc1d15 | 3.97E−18 | 0.432216558 | 0.461 | 0.319 | 9.86E−14 | A6 | Tbc1d15 |
| Dst | 3.98E−18 | 0.447341116 | 0.43 | 0.276 | 9.87E−14 | A6 | Dst |
| Lmo4 | 4.29E−18 | 0.296248817 | 0.308 | 0.162 | 1.06E−13 | A6 | Lmo4 |
| Egr1 | 5.65E−18 | 0.537039177 | 0.589 | 0.481 | 1.40E−13 | A6 | Egr1 |
| Hnrnpdl | 8.07E−18 | 0.399328599 | 0.406 | 0.254 | 2.00E−13 | A6 | Hnrnpdl |
| Aff4 | 1.70E−17 | 0.536338474 | 0.537 | 0.423 | 4.23E−13 | A6 | Aff4 |
| Gn12 | 1.83E−17 | 0.358031458 | 0.384 | 0.235 | 4.55E−13 | A6 | Gn12 |
| Lrrfip1 | 2.21E−17 | 0.410825691 | 0.37 | 0.221 | 5.49E−13 | A6 | Lrrfip1 |
| Aass | 2.35E−17 | 0.40708364 | 0.747 | 0.763 | 5.83E−13 | A6 | Aass |
| Kif5b | 8.07E−17 | 0.452231438 | 0.494 | 0.379 | 2.00E−12 | A6 | Kif5b |
| Wbp1l | 1.01E−16 | 0.389703337 | 0.697 | 0.683 | 2.52E−12 | A6 | Wbp1l |
| Stom | 1.12E−16 | 0.299958852 | 0.329 | 0.184 | 2.77E−12 | A6 | Stom |
| Arhgef12 | 1.63E−16 | 0.485612023 | 0.578 | 0.494 | 4.05E−12 | A6 | Arhgef12 |
| Pxk | 4.69E−16 | 0.265227811 | 0.303 | 0.161 | 1.16E−11 | A6 | Pxk |
| Prrc2c | 6.21E−16 | 0.390594415 | 0.415 | 0.266 | 1.54E−11 | A6 | Prrc2c |
| Sds | 6.91E−16 | 0.351547188 | 0.73 | 0.632 | 1.72E−11 | A6 | Sds |
| Mybbp1a | 7.16E−16 | 0.401802664 | 0.396 | 0.263 | 1.78E−11 | A6 | Mybbp1a |
| Wdr43 | 8.36E−16 | 0.308292044 | 0.353 | 0.208 | 2.08E−11 | A6 | Wdr43 |
| Mmp14 | 9.37E−16 | 0.257143847 | 0.253 | 0.125 | 2.33E−11 | A6 | Mmp14 |
| Ptpn2 | 9.68E−16 | 0.270962207 | 0.255 | 0.124 | 2.40E−11 | A6 | Ptpn2 |
| Mak16 | 1.01E−15 | 0.28973233 | 0.267 | 0.134 | 2.51E−11 | A6 | Mak16 |
| Plk3 | 1.73E−15 | 0.415826723 | 0.413 | 0.269 | 4.29E−11 | A6 | Plk3 |
| Tm4sf4 | 2.46E−15 | 0.400558068 | 0.484 | 0.363 | 6.11E−11 | A6 | Tm4sf4 |
| Cyp39a1 | 4.02E−15 | 0.299582361 | 0.305 | 0.171 | 9.99E−11 | A6 | Cyp39a1 |
| Nedd4l | 4.88E−15 | 0.263544573 | 0.332 | 0.189 | 1.21E−10 | A6 | Nedd4l |
| Ptpn12 | 6.24E−15 | 0.309327486 | 0.263 | 0.135 | 1.55E−10 | A6 | Ptpn12 |
| Map3k5 | 6.60E−15 | 0.313064805 | 0.346 | 0.206 | 1.64E−10 | A6 | Map3k5 |
| Nars | 7.95E−15 | 0.368685334 | 0.606 | 0.539 | 1.97E−10 | A6 | Nars |
| Ptp4a1 | 9.30E−15 | 0.251709471 | 0.334 | 0.192 | 2.31E−10 | A6 | Ptp4a1 |
| Gm24245 | 1.17E−14 | 0.498425809 | 0.422 | 0.269 | 2.89E−10 | A6 | Gm24245 |
| Ifrd1 | 1.21E−14 | 0.476476582 | 0.365 | 0.226 | 3.00E−10 | A6 | Ifrd1 |
| AA474408 | 1.38E−14 | 0.266419343 | 0.274 | 0.142 | 3.42E−10 | A6 | AA474408 |
| Zyx | 2.09E−14 | 0.279857668 | 0.263 | 0.137 | 5.20E−10 | A6 | Zyx |
| Smg1 | 2.32E−14 | 0.304468325 | 0.265 | 0.141 | 5.76E−10 | A6 | Smg1 |
| Man2a1 | 3.27E−14 | 0.343929917 | 0.671 | 0.659 | 8.13E−10 | A6 | Man2a1 |
| Lrrfip2 | 3.29E−14 | 0.356490426 | 0.384 | 0.247 | 8.16E−10 | A6 | Lrrfip2 |
| Ppp1r12a | 3.38E−14 | 0.325164021 | 0.317 | 0.182 | 8.39E−10 | A6 | Ppp1r12a |
| Chd1 | 4.71E−14 | 0.314128487 | 0.377 | 0.243 | 1.17E−09 | A6 | Chd1 |
| Lpgat1 | 5.37E−14 | 0.443498962 | 0.618 | 0.593 | 1.33E−09 | A6 | Lpgat1 |
| Palld | 6.63E−14 | 0.3083699 | 0.353 | 0.218 | 1.65E−09 | A6 | Palld |
| H2-D1 | 6.78E−14 | 0.298627546 | 0.766 | 0.781 | 1.68E−09 | A6 | H2-D1 |
| Grn | 7.22E−14 | 0.373811784 | 0.582 | 0.499 | 1.79E−09 | A6 | Grn |
| Fus | 1.11E−13 | 0.338818431 | 0.394 | 0.267 | 2.76E−09 | A6 | Fus |
| Litaf | 1.26E−13 | 0.351166994 | 0.451 | 0.34 | 3.13E−09 | A6 | Litaf |
| Mki67ip | 1.50E−13 | 0.312167827 | 0.344 | 0.212 | 3.73E−09 | A6 | Mki67ip |
| Lcp1 | 2.39E−13 | 0.464895097 | 0.492 | 0.365 | 5.94E−09 | A6 | Lcp1 |
| mt-Rnr1 | 2.60E−13 | 0.390086647 | 1 | 1 | 6.45E−09 | A6 | mt-Rnr1 |
| Ctsb | 2.79E−13 | 0.469114925 | 0.804 | 0.82 | 6.94E−09 | A6 | Ctsb |
| Sptbn1 | 3.21E−13 | 0.376219831 | 0.439 | 0.325 | 7.98E−09 | A6 | Sptbn1 |
| Sfpq | 5.31E−13 | 0.34484544 | 0.406 | 0.277 | 1.32E−08 | A6 | Sfpq |
| Tgm2 | 5.71E−13 | 0.362144298 | 0.446 | 0.314 | 1.42E−08 | A6 | Tgm2 |
| Eif4g1 | 1.45E−12 | 0.314414152 | 0.723 | 0.719 | 3.60E−08 | A6 | Eif4g1 |
| Fn1 | 2.32E−12 | 0.28519688 | 0.847 | 0.866 | 5.76E−08 | A6 | Fn1 |
| Purb | 2.57E−12 | 0.310815573 | 0.341 | 0.219 | 6.37E−08 | A6 | Purb |
| Phlda1 | 2.84E−12 | 0.472335823 | 0.494 | 0.429 | 7.05E−08 | A6 | Phlda1 |
| Atp2a2 | 3.95E−12 | 0.364622996 | 0.616 | 0.575 | 9.81E−08 | A6 | Atp2a2 |
| Etnppl | 6.99E−12 | 0.30440999 | 0.706 | 0.62 | 1.74E−07 | A6 | Etnppl |
| Akap13 | 7.44E−12 | 0.443592591 | 0.406 | 0.282 | 1.85E−07 | A6 | Akap13 |
| Wdr26 | 1.07E−11 | 0.372353239 | 0.549 | 0.472 | 2.66E−07 | A6 | Wdr26 |
| Huwe1 | 1.08E−11 | 0.333579982 | 0.492 | 0.38 | 2.69E−07 | A6 | Huwe1 |
| Cald1 | 1.16E−11 | 0.293474512 | 0.766 | 0.795 | 2.89E−07 | A6 | Cald1 |
| Ankrd11 | 1.30E−11 | 0.371489147 | 0.389 | 0.263 | 3.24E−07 | A6 | Ankrd11 |
| Mbnl1 | 2.38E−11 | 0.34826585 | 0.492 | 0.394 | 5.91E−07 | A6 | Mbnl1 |
| Mug2 | 2.45E−11 | 0.277303056 | 0.776 | 0.77 | 6.09E−07 | A6 | Mug2 |
| Rn7sk | 2.61E−11 | 0.277601787 | 0.339 | 0.212 | 6.48E−07 | A6 | Rn7sk |
| Anxa5 | 2.84E−11 | 0.357988662 | 0.391 | 0.278 | 7.05E−07 | A6 | Anxa5 |
| Metap2 | 4.08E−11 | 0.327962496 | 0.453 | 0.353 | 1.01E−06 | A6 | Metap2 |
| Shb | 4.85E−11 | 0.283085429 | 0.382 | 0.264 | 1.20E−06 | A6 | Shb |
| Gsr | 4.90E−11 | 0.392388862 | 0.453 | 0.346 | 1.22E−06 | A6 | Gsr |
| Gsk3b | 5.45E−11 | 0.35404888 | 0.384 | 0.272 | 1.35E−06 | A6 | Gsk3b |
| Kdelr2 | 5.86E−11 | 0.36902044 | 0.484 | 0.413 | 1.45E−06 | A6 | Kdelr2 |
| Tjp1 | 1.36E−10 | 0.252895758 | 0.251 | 0.142 | 3.37E−06 | A6 | Tjp1 |
| Sf1 | 2.46E−10 | 0.277710053 | 0.351 | 0.233 | 6.11E−06 | A6 | Sf1 |
| Dhx9 | 3.77E−10 | 0.305614472 | 0.427 | 0.319 | 9.37E−06 | A6 | Dhx9 |
| Smap2 | 4.08E−10 | 0.317056211 | 0.363 | 0.246 | 1.01E−05 | A6 | Smap2 |
| Chmp4b | 4.61E−10 | 0.293914095 | 0.442 | 0.344 | 1.14E−05 | A6 | Chmp4b |
| Cxcl1 | 5.14E−10 | 0.309204773 | 0.444 | 0.334 | 1.28E−05 | A6 | Cxcl1 |
| Snx10 | 5.28E−10 | 0.264074664 | 0.341 | 0.232 | 1.31E−05 | A6 | Snx10 |
| Pacsin2 | 5.77E−10 | 0.342565625 | 0.37 | 0.264 | 1.43E−05 | A6 | Pacsin2 |
| Cast | 7.34E−10 | 0.341920461 | 0.399 | 0.293 | 1.82E−05 | A6 | Cast |
| Akap11 | 7.52E−10 | 0.297231801 | 0.351 | 0.24 | 1.87E−05 | A6 | Akap11 |
| Srsf11 | 7.58E−10 | 0.354232945 | 0.418 | 0.313 | 1.88E−05 | A6 | Srsf11 |
| Reep3 | 9.04E−10 | 0.335070143 | 0.535 | 0.502 | 2.24E−05 | A6 | Reep3 |
| mmu-mir-6236 | 1.28E−09 | 0.420467035 | 0.831 | 0.853 | 3.19E−05 | A6 | mmu-mir-6236 |
| Atf4 | 2.02E−09 | 0.286714844 | 0.58 | 0.505 | 5.02E−05 | A6 | Atf4 |
| Clic4 | 2.32E−09 | 0.2810953 | 0.368 | 0.265 | 5.77E−05 | A6 | Clic4 |
| Ubr4 | 2.43E−09 | 0.291606896 | 0.411 | 0.302 | 6.03E−05 | A6 | Ubr4 |
| Por | 2.58E−09 | 0.268724257 | 0.704 | 0.721 | 6.42E−05 | A6 | Por |
| Bptf | 2.63E−09 | 0.281824549 | 0.332 | 0.222 | 6.53E−05 | A6 | Bptf |
| Zbtb20 | 2.91E−09 | 0.378048195 | 0.566 | 0.519 | 7.23E−05 | A6 | Zbtb20 |
| Dync1h1 | 3.26E−09 | 0.341961739 | 0.379 | 0.275 | 8.10E−05 | A6 | Dync1h1 |
| Rlim | 3.27E−09 | 0.275758769 | 0.305 | 0.199 | 8.12E−05 | A6 | Rlim |
| Birc6 | 4.85E−09 | 0.292199154 | 0.446 | 0.34 | 0.000120422 | A6 | Birc6 |
| Atp1b1 | 5.41E−09 | 0.313967935 | 0.391 | 0.294 | 0.000134226 | A6 | Atp1b1 |
| Slc25a47 | 6.75E−09 | 0.288429921 | 0.869 | 0.931 | 0.000167694 | A6 | Slc25a47 |
| Slc25a51 | 7.23E−09 | 0.253289156 | 0.308 | 0.203 | 0.000179545 | A6 | Slc25a51 |
| Jhdm1d | 7.29E−09 | 0.254152235 | 0.279 | 0.177 | 0.000181067 | A6 | Jhdm1d |
| Steap4 | 9.00E−09 | 0.36530445 | 0.37 | 0.275 | 0.000223544 | A6 | Steap4 |
| Mapkapk2 | 9.71E−09 | 0.30529864 | 0.396 | 0.294 | 0.000241093 | A6 | Mapkapk2 |
| Gtf2a2 | 1.09E−08 | 0.265404263 | 0.379 | 0.28 | 0.000271551 | A6 | Gtf2a2 |
| Slc4a4 | 1.12E−08 | 0.295268979 | 0.513 | 0.439 | 0.000278519 | A6 | Slc4a4 |
| Tbl1xr1 | 1.79E−08 | 0.255321469 | 0.341 | 0.246 | 0.000443552 | A6 | Tbl1xr1 |
| Sbno1 | 1.89E−08 | 0.296206562 | 0.394 | 0.301 | 0.000469821 | A6 | Sbno1 |
| Nrp1 | 1.98E−08 | 0.320317373 | 0.391 | 0.295 | 0.00049277 | A6 | Nrp1 |
| Tpr | 2.01E−08 | 0.352440039 | 0.501 | 0.429 | 0.000499279 | A6 | Tpr |
| Kif21a | 2.01E−08 | 0.333362777 | 0.461 | 0.377 | 0.000500319 | A6 | Kif21a |
| Mdm2 | 2.06E−08 | 0.275809807 | 0.305 | 0.204 | 0.000510843 | A6 | Mdm2 |
| Ddx6 | 2.13E−08 | 0.347331371 | 0.527 | 0.46 | 0.000529761 | A6 | Ddx6 |
| Rbm25 | 2.41E−08 | 0.365024904 | 0.461 | 0.369 | 0.000598306 | A6 | Rbm25 |
| Uhrf1bp1l | 2.44E−08 | 0.319283953 | 0.399 | 0.306 | 0.000606861 | A6 | Uhrf1bp1l |
| Cep85 | 3.36E−08 | 0.270833691 | 0.313 | 0.215 | 0.00083347 | A6 | Cep85 |
| Hmgcr | 3.52E−08 | 0.344898662 | 0.425 | 0.343 | 0.000873878 | A6 | Hmgcr |
| Saa2 | 4.22E−08 | 0.38920839 | 0.315 | 0.221 | 0.001047242 | A6 | Saa2 |
| Tnks2 | 5.05E−08 | 0.267384994 | 0.358 | 0.265 | 0.001254373 | A6 | Tnks2 |
| Net1 | 5.81E−08 | 0.320110712 | 0.408 | 0.324 | 0.001441888 | A6 | Net1 |
| Rangap1 | 6.81E−08 | 0.271467828 | 0.387 | 0.298 | 0.001690824 | A6 | Rangap1 |
| Atp13a3 | 8.04E−08 | 0.298039121 | 0.532 | 0.494 | 0.001997402 | A6 | Atp13a3 |
| Tmed5 | 9.87E−08 | 0.262840646 | 0.594 | 0.587 | 0.002451811 | A6 | Tmed5 |
| Atp2b1 | 1.14E−07 | 0.289377372 | 0.334 | 0.239 | 0.002830088 | A6 | Atp2b1 |
| Ell2 | 1.24E−07 | 0.307296281 | 0.535 | 0.508 | 0.003081707 | A6 | Ell2 |
| Eif4a1 | 1.50E−07 | 0.260785213 | 0.418 | 0.331 | 0.003715312 | A6 | Eif4a1 |
| Macf1 | 1.58E−07 | 0.347805477 | 0.377 | 0.287 | 0.003914779 | A6 | Macf1 |
| Srrm2 | 1.83E−07 | 0.286594156 | 0.456 | 0.372 | 0.004531922 | A6 | Srrm2 |
| Brd2 | 1.86E−07 | 0.459822776 | 0.542 | 0.493 | 0.004608791 | A6 | Brd2 |
| Ipo7 | 1.95E−07 | 0.267191431 | 0.334 | 0.241 | 0.004846455 | A6 | Ipo7 |
| Cap1 | 2.19E−07 | 0.285316115 | 0.356 | 0.261 | 0.005437156 | A6 | Cap1 |
| Lrrc59 | 2.71E−07 | 0.30237878 | 0.422 | 0.354 | 0.006720029 | A6 | Lrrc59 |
| Scaf11 | 3.09E−07 | 0.287560725 | 0.449 | 0.382 | 0.007680311 | A6 | Scaf11 |
| Phf20l1 | 3.47E−07 | 0.265681682 | 0.372 | 0.282 | 0.008627157 | A6 | Phf20l1 |
| Hipk3 | 3.52E−07 | 0.260488512 | 0.396 | 0.309 | 0.008752398 | A6 | Hipk3 |
| Prpf19 | 3.65E−07 | 0.256584518 | 0.432 | 0.356 | 0.009072104 | A6 | Prpf19 |
| Actr3 | 4.09E−07 | 0.263828451 | 0.36 | 0.273 | 0.0101555 | A6 | Actr3 |
| Gfra1 | 5.26E−07 | 0.275822348 | 0.599 | 0.583 | 0.013059775 | A6 | Gfra1 |
| Dnajc5 | 5.58E−07 | 0.263545549 | 0.372 | 0.294 | 0.013867302 | A6 | Dnajc5 |
| Gm5424 | 7.53E−07 | 0.264697117 | 0.411 | 0.325 | 0.018693371 | A6 | Gm5424 |
| Ppip5k2 | 7.82E−07 | 0.300238752 | 0.344 | 0.254 | 0.019412805 | A6 | Ppip5k2 |
| Yap1 | 8.00E−07 | 0.261525975 | 0.375 | 0.288 | 0.019872963 | A6 | Yap1 |
| mt-Co1 | 1.20E−06 | 0.351015168 | 0.998 | 0.988 | 0.029744292 | A6 | mt-Co1 |
| Mgea5 | 1.25E−06 | 0.290424441 | 0.413 | 0.333 | 0.031079611 | A6 | Mgea5 |
| Kpna1 | 1.55E−06 | 0.255454573 | 0.363 | 0.286 | 0.038470274 | A6 | Kpna1 |
| Atf6 | 1.79E−06 | 0.292221667 | 0.432 | 0.365 | 0.04443216 | A6 | Atf6 |
| Ivns1abp | 1.94E−06 | 0.275508294 | 0.446 | 0.398 | 0.048239404 | A6 | Ivns1abp |
| Dnaja2 | 2.46E−06 | 0.299312472 | 0.516 | 0.482 | 0.06120923 | A6 | Dnaja2 |
| Ctsc | 2.59E−06 | 0.257615328 | 0.403 | 0.321 | 0.064323031 | A6 | Ctsc |
| Kdm5a | 2.84E−06 | 0.310177967 | 0.308 | 0.221 | 0.070537642 | A6 | Kdm5a |
| Abce1 | 2.87E−06 | 0.250233228 | 0.437 | 0.365 | 0.071298568 | A6 | Abce1 |
| Rsl1d1 | 3.31E−06 | 0.274880418 | 0.47 | 0.423 | 0.08207937 | A6 | Rsl1d1 |
| Eprs | 4.18E−06 | 0.297658536 | 0.566 | 0.558 | 0.10382179 | A6 | Eprs |
| Hnrnpu | 5.29E−06 | 0.250578107 | 0.625 | 0.651 | 0.131375216 | A6 | Hnrnpu |
| H6pd | 5.50E−06 | 0.256150539 | 0.623 | 0.652 | 0.136642791 | A6 | H6pd |
| mt-Nd5 | 6.52E−06 | 0.341125985 | 0.983 | 0.969 | 0.161943697 | A6 | mt-Nd5 |
| Rnf169 | 7.00E−06 | 0.296323635 | 0.315 | 0.235 | 0.173720819 | A6 | Rnf169 |
| Rap1b | 7.33E−06 | 0.284122894 | 0.406 | 0.335 | 0.181944357 | A6 | Rap1b |
| Chuk | 8.66E−06 | 0.295478719 | 0.523 | 0.495 | 0.214923523 | A6 | Chuk |
| Sf3b1 | 9.81E−06 | 0.250611588 | 0.508 | 0.475 | 0.243582219 | A6 | Sf3b1 |
| Serinc3 | 1.22E−05 | 0.2811911 | 0.525 | 0.498 | 0.303917936 | A6 | Serinc3 |
| Pi4k2a | 1.25E−05 | 0.327022483 | 0.425 | 0.381 | 0.310307299 | A6 | Pi4k2a |
| Alcam | 1.35E−05 | 0.264774262 | 0.396 | 0.329 | 0.33420498 | A6 | Alcam |
| Gadd45a | 1.39E−05 | 0.282094324 | 0.251 | 0.178 | 0.344761555 | A6 | Gadd45a |
| Diap1 | 1.79E−05 | 0.281572774 | 0.382 | 0.306 | 0.444812392 | A6 | Diap1 |
| Ddx3y | 3.35E−05 | 0.277824135 | 0.47 | 0.43 | 0.83189176 | A6 | Ddx3y |
| Eif4b | 3.52E−05 | 0.278878236 | 0.542 | 0.521 | 0.873426916 | A6 | Eif4b |
| Nploc4 | 5.58E−05 | 0.251530839 | 0.351 | 0.28 | 1 | A6 | Nploc4 |
| Nucks1 | 7.26E−05 | 0.260380087 | 0.532 | 0.517 | 1 | A6 | Nucks1 |
| Tab2 | 7.82E−05 | 0.293074476 | 0.425 | 0.374 | 1 | A6 | Tab2 |
| Slc35d1 | 8.85E−05 | 0.253786261 | 0.489 | 0.496 | 1 | A6 | Slc35d1 |
| Abcc2 | 8.98E−05 | 0.260028664 | 0.632 | 0.654 | 1 | A6 | Abcc2 |
| Atp1a1 | 9.45E−05 | 0.291312422 | 0.53 | 0.538 | 1 | A6 | Atp1a1 |
| Slc40a1 | 0.00029608 | 0.349948116 | 0.456 | 0.417 | 1 | A6 | Slc40a1 |
| Zfp106 | 0.000319641 | 0.270672764 | 0.391 | 0.333 | 1 | A6 | Zfp106 |
| Ier2 | 0.000367025 | 0.250748851 | 0.551 | 0.517 | 1 | A6 | Ier2 |
| Trip11 | 0.000589236 | 0.287017056 | 0.341 | 0.284 | 1 | A6 | Trip11 |
| Srsf5 | 0.000641609 | 0.253489779 | 0.387 | 0.34 | 1 | A6 | Srsf5 |
| Spag9 | 0.001729185 | 0.25323185 | 0.415 | 0.373 | 1 | A6 | Spag9 |
| Tln1 | 0.00250885 | 0.293695038 | 0.31 | 0.259 | 1 | A6 | Tln1 |
| Riok3 | 0.003018499 | 0.258919292 | 0.43 | 0.406 | 1 | A6 | Riok3 |
| mt-Nd6 | 0.003486244 | 0.290617021 | 0.527 | 0.502 | 1 | A6 | mt-Nd6 |
| Gm269241 | 0 | 1.253161452 | 1 | 1 | 0 | A24 | Gm26924 |
| n-R5-8s11 | 0 | 1.150811396 | 0.522 | 0.075 | 0 | A24 | n-R5-8s1 |
| Grip2 | 0 | 0.877370568 | 0.475 | 0.003 | 0 | A24 | Grip2 |
| Fga1 | 0 | 0.762040686 | 0.998 | 0.996 | 0 | A24 | Fga |
| Fgb1 | 0 | 0.681029638 | 1 | 0.999 | 0 | A24 | Fgb |
| Fgg1 | 0 | 0.633396491 | 0.999 | 0.993 | 0 | A24 | Fgg |
| Lars21 | 1.08E−286 | 1.493131216 | 0.998 | 0.995 | 2.69E−282 | A24 | Lars2 |
| Sephs21 | 1.58E−286 | 0.853566054 | 0.949 | 0.785 | 3.91E−282 | A24 | Sephs2 |
| Fgl1 | 5.87E−275 | 0.874063594 | 0.938 | 0.799 | 1.46E−270 | A24 | Fgl1 |
| Gm24601 | 4.02E−274 | 0.932441655 | 0.404 | 0.047 | 9.99E−270 | A24 | Gm24601 |
| Gm155641 | 3.89E−272 | 1.555434308 | 0.982 | 0.965 | 9.67E−268 | A24 | Gm15564 |
| Calr1 | 5.92E−193 | 0.611584258 | 0.952 | 0.882 | 1.47E−188 | A24 | Calr |
| Pdia31 | 3.00E−171 | 0.642410377 | 0.895 | 0.805 | 7.45E−167 | A24 | Pdia3 |
| Hyou1 | 1.92E−169 | 0.843594109 | 0.717 | 0.423 | 4.77E−165 | A24 | Hyou1 |
| Saa21 | 1.56E−157 | 1.00910228 | 0.52 | 0.172 | 3.88E−153 | A24 | Saa2 |
| Hsp90b11 | 2.63E−155 | 0.545125445 | 0.954 | 0.948 | 6.53E−151 | A24 | Hsp90b1 |
| Sepp1 | 5.04E−153 | 0.326034322 | 1 | 1 | 1.25E−148 | A24 | Sepp1 |
| Hspa5 | 1.66E−152 | 0.489951106 | 0.974 | 0.954 | 4.12E−148 | A24 | Hspa5 |
| Orm1 | 6.97E−135 | 0.466115424 | 0.963 | 0.942 | 1.73E−130 | A24 | Orm1 |
| Itih4 | 2.79E−129 | 0.46889806 | 0.974 | 0.956 | 6.93E−125 | A24 | Itih4 |
| Egfr | 2.69E−128 | 0.62342268 | 0.882 | 0.802 | 6.67E−124 | A24 | Egfr |
| Glud1 | 1.70E−115 | 0.42115272 | 0.958 | 0.921 | 4.22E−111 | A24 | Glud1 |
| Hp | 2.71E−113 | 0.364354354 | 0.998 | 0.999 | 6.73E−109 | A24 | Hp |
| Mup71 | 1.68E−111 | 0.268129829 | 0.985 | 0.965 | 4.16E−107 | A24 | Mup7 |
| Serpina101 | 2.00E−108 | 0.570729192 | 0.813 | 0.667 | 4.97E−104 | A24 | Serpina10 |
| Rnase41 | 2.35E−108 | 0.554006694 | 0.963 | 0.898 | 5.83E−104 | A24 | Rnase4 |
| Scd11 | 2.78E−104 | 0.558572468 | 0.98 | 0.925 | 6.90E−100 | A24 | Scd1 |
| Eef1a1 | 2.17E−101 | 0.456135198 | 0.919 | 0.858 | 5.38E−97 | A24 | Eef1a1 |
| Saa1 | 2.48E−100 | 0.781172408 | 0.792 | 0.549 | 6.16E−96 | A24 | Saa1 |
| C8b1 | 1.53E−99 | 0.440139433 | 0.898 | 0.871 | 3.79E−95 | A24 | C8b |
| Hdlbp | 2.71E−98 | 0.527847395 | 0.853 | 0.753 | 6.72E−94 | A24 | Hdlbp |
| Cyp2c701 | 7.78E−90 | 0.479527584 | 0.89 | 0.823 | 1.93E−85 | A24 | Cyp2c70 |
| P4hb1 | 2.74E−89 | 0.332542787 | 0.973 | 0.945 | 6.82E−85 | A24 | P4hb |
| Apcs | 5.47E−89 | 0.605911685 | 0.738 | 0.556 | 1.36E−84 | A24 | Apcs |
| Creld2 | 5.15E−88 | 0.594060178 | 0.583 | 0.352 | 1.28E−83 | A24 | Creld2 |
| C91 | 2.56E−84 | 0.375762746 | 0.952 | 0.926 | 6.35E−80 | A24 | C9 |
| Orm2 | 5.62E−84 | 0.304652907 | 0.285 | 0.081 | 1.40E−79 | A24 | Orm2 |
| Ssr3 | 1.77E−83 | 0.486117928 | 0.765 | 0.642 | 4.40E−79 | A24 | Ssr3 |
| Pzp | 4.35E−83 | 0.354307454 | 0.984 | 0.988 | 1.08E−78 | A24 | Pzp |
| C31 | 2.09E−82 | 0.323813995 | 0.996 | 0.993 | 5.18E−78 | A24 | C3 |
| Arrdc31 | 1.19E−81 | 0.498916524 | 0.52 | 0.272 | 2.97E−77 | A24 | Arrdc3 |
| Saa41 | 1.26E−81 | 0.476275836 | 0.823 | 0.734 | 3.13E−77 | A24 | Saa4 |
| Fn11 | 3.93E−80 | 0.553626994 | 0.905 | 0.858 | 9.76E−76 | A24 | Fn1 |
| Cfh | 3.78E−79 | 0.454722845 | 0.921 | 0.904 | 9.38E−75 | A24 | Cfh |
| Cat | 3.96E−78 | 0.285637918 | 0.978 | 0.977 | 9.84E−74 | A24 | Cat |
| Actb1 | 4.53E−78 | 0.322827073 | 0.919 | 0.895 | 1.12E−73 | A24 | Actb |
| Pdia41 | 2.83E−76 | 0.520868525 | 0.733 | 0.616 | 7.02E−72 | A24 | Pdia4 |
| Sc5d1 | 4.88E−76 | 0.398810219 | 0.879 | 0.797 | 1.21E−71 | A24 | Sc5d |
| Steap41 | 5.67E−76 | 0.466342007 | 0.481 | 0.243 | 1.41E−71 | A24 | Steap4 |
| C8a | 1.03E−72 | 0.424002753 | 0.902 | 0.877 | 2.55E−68 | A24 | C8a |
| Pgrmc1 | 1.30E−70 | 0.307565593 | 0.953 | 0.947 | 3.22E−66 | A24 | Pgrmc1 |
| Itih31 | 3.45E−70 | 0.393234979 | 0.931 | 0.909 | 8.58E−66 | A24 | Itih3 |
| Nudt71 | 3.41E−69 | 0.34725512 | 0.884 | 0.74 | 8.47E−65 | A24 | Nudt7 |
| Hc | 5.21E−67 | 0.388992856 | 0.905 | 0.904 | 1.29E−62 | A24 | Hc |
| Tram1 | 1.87E−66 | 0.42942297 | 0.7 | 0.545 | 4.63E−62 | A24 | Tram1 |
| Myh91 | 1.24E−65 | 0.491944623 | 0.595 | 0.388 | 3.08E−61 | A24 | Myh9 |
| Mup121 | 1.65E−65 | 0.453275054 | 0.799 | 0.759 | 4.10E−61 | A24 | Mup12 |
| Gm269171 | 8.37E−65 | 0.787057247 | 0.391 | 0.183 | 2.08E−60 | A24 | Gm26917 |
| Dnajc3 | 2.12E−64 | 0.38898869 | 0.85 | 0.808 | 5.26E−60 | A24 | Dnajc3 |
| Hao1 | 7.24E−61 | 0.391622737 | 0.83 | 0.767 | 1.80E−56 | A24 | Hao1 |
| Pdia61 | 8.25E−60 | 0.420925025 | 0.779 | 0.702 | 2.05E−55 | A24 | Pdia6 |
| Apob1 | 9.57E−60 | 0.353979053 | 0.983 | 0.99 | 2.38E−55 | A24 | Apob |
| Manf | 2.64E−58 | 0.465253736 | 0.632 | 0.478 | 6.55E−54 | A24 | Manf |
| Pdcd41 | 4.76E−58 | 0.46790935 | 0.677 | 0.542 | 1.18E−53 | A24 | Pdcd4 |
| AI1823711 | 9.61E−58 | 0.423055602 | 0.747 | 0.654 | 2.39E−53 | A24 | AI182371 |
| Comt1 | 5.49E−57 | 0.302019368 | 0.928 | 0.92 | 1.36E−52 | A24 | Comt |
| Cpb21 | 2.17E−56 | 0.331551897 | 0.879 | 0.827 | 5.40E−52 | A24 | Cpb2 |
| Serp1 | 2.18E−55 | 0.402045023 | 0.809 | 0.725 | 5.42E−51 | A24 | Serp1 |
| Cml21 | 5.80E−55 | 0.34556361 | 0.896 | 0.849 | 1.44E−50 | A24 | Cml2 |
| Elovl2 | 1.73E−53 | 0.380671427 | 0.826 | 0.751 | 4.29E−49 | A24 | Elovl2 |
| mmu-mir-62361 | 6.37E−53 | 0.845892758 | 0.896 | 0.843 | 1.58E−48 | A24 | mmu-mir-6236 |
| Iigp11 | 1.35E−52 | 0.333549341 | 0.832 | 0.798 | 3.36E−48 | A24 | Iigp1 |
| Calm1 | 1.03E−50 | 0.370158531 | 0.794 | 0.759 | 2.56E−46 | A24 | Calm1 |
| Cps1 | 2.48E−50 | 0.277513678 | 0.982 | 0.977 | 6.16E−46 | A24 | Cps1 |
| Eif4g21 | 6.14E−50 | 0.351474078 | 0.8 | 0.739 | 1.52E−45 | A24 | Eif4g2 |
| Acox1 | 1.63E−48 | 0.274311383 | 0.966 | 0.968 | 4.05E−44 | A24 | Acox1 |
| Fndc3b1 | 3.80E−48 | 0.320238213 | 0.291 | 0.125 | 9.44E−44 | A24 | Fndc3b |
| Slc41a2 | 1.01E−47 | 0.28795823 | 0.302 | 0.132 | 2.51E−43 | A24 | Slc41a2 |
| Nr1d2 | 5.02E−47 | 0.385472609 | 0.442 | 0.263 | 1.25E−42 | A24 | Nr1d2 |
| Insig1 | 1.30E−46 | 0.412470778 | 0.626 | 0.487 | 3.23E−42 | A24 | Insig1 |
| Aox31 | 1.16E−45 | 0.348891224 | 0.916 | 0.896 | 2.88E−41 | A24 | Aox3 |
| Spcs3 | 6.70E−45 | 0.390709307 | 0.483 | 0.325 | 1.66E−40 | A24 | Spcs3 |
| Atxn1 | 2.39E−44 | 0.325041158 | 0.311 | 0.148 | 5.93E−40 | A24 | Atxn1 |
| Cmah | 3.14E−44 | 0.424294686 | 0.607 | 0.472 | 7.80E−40 | A24 | Cmah |
| Pnpla7 | 4.00E−44 | 0.396590342 | 0.678 | 0.561 | 9.93E−40 | A24 | Pnpla7 |
| Dnajc12 | 1.33E−42 | 0.324049682 | 0.275 | 0.125 | 3.31E−38 | A24 | Dnajc12 |
| Atp8b1 | 3.45E−42 | 0.361014487 | 0.391 | 0.221 | 8.56E−38 | A24 | Atp8b1 |
| Hspa13 | 3.59E−42 | 0.341967618 | 0.404 | 0.237 | 8.92E−38 | A24 | Hspa13 |
| AA4744081 | 1.41E−41 | 0.414816707 | 0.278 | 0.126 | 3.51E−37 | A24 | AA474408 |
| Canx | 1.09E−40 | 0.277059295 | 0.872 | 0.862 | 2.70E−36 | A24 | Canx |
| Tmed7 | 1.35E−40 | 0.371496345 | 0.563 | 0.417 | 3.35E−36 | A24 | Tmed7 |
| Il13ra1 | 1.42E−40 | 0.288909598 | 0.33 | 0.168 | 3.53E−36 | A24 | Il13ra1 |
| Sdf2l1 | 2.54E−40 | 0.307771718 | 0.412 | 0.24 | 6.31E−36 | A24 | Sdf2l1 |
| Acly | 5.13E−40 | 0.410128052 | 0.597 | 0.448 | 1.27E−35 | A24 | Acly |
| Cxcl11 | 8.60E−40 | 0.453014277 | 0.482 | 0.315 | 2.14E−35 | A24 | Cxcl1 |
| Prg4 | 3.70E−39 | 0.400695342 | 0.421 | 0.254 | 9.19E−35 | A24 | Prg4 |
| Adh41 | 9.82E−39 | 0.391972485 | 0.667 | 0.538 | 2.44E−34 | A24 | Adh4 |
| Lrg11 | 4.41E−38 | 0.30956022 | 0.849 | 0.786 | 1.09E−33 | A24 | Lrg1 |
| Hsd17b21 | 1.09E−37 | 0.323137514 | 0.758 | 0.682 | 2.72E−33 | A24 | Hsd17b2 |
| Hsd3b51 | 1.25E−36 | 0.30091435 | 0.638 | 0.451 | 3.10E−32 | A24 | Hsd3b5 |
| Hs3st3b1 | 3.00E−36 | 0.321902053 | 0.442 | 0.286 | 7.44E−32 | A24 | Hs3st3b1 |
| Rpn1 | 3.41E−36 | 0.399145856 | 0.605 | 0.494 | 8.47E−32 | A24 | Rpn1 |
| Tmem30a | 9.46E−36 | 0.305621509 | 0.766 | 0.712 | 2.35E−31 | A24 | Tmem30a |
| Rrbp1 | 2.26E−35 | 0.296754853 | 0.823 | 0.794 | 5.62E−31 | A24 | Rrbp1 |
| Cyb5r3 | 9.28E−35 | 0.309656426 | 0.8 | 0.757 | 2.30E−30 | A24 | Cyb5r3 |
| Hnf4a1 | 9.32E−34 | 0.254948257 | 0.85 | 0.799 | 2.31E−29 | A24 | Hnf4a |
| Fos | 1.67E−33 | 0.363187233 | 0.654 | 0.54 | 4.16E−29 | A24 | Fos |
| Abca11 | 3.07E−33 | 0.383089812 | 0.556 | 0.428 | 7.62E−29 | A24 | Abca1 |
| Cltc1 | 1.19E−32 | 0.33966836 | 0.61 | 0.485 | 2.96E−28 | A24 | Cltc |
| Qsox1 | 6.53E−32 | 0.281749774 | 0.784 | 0.741 | 1.62E−27 | A24 | Qsox1 |
| Lrp1 | 2.33E−31 | 0.483743655 | 0.694 | 0.626 | 5.78E−27 | A24 | Lrp1 |
| Mlec | 2.33E−31 | 0.340526351 | 0.496 | 0.361 | 5.78E−27 | A24 | Mlec |
| Ssr1 | 3.21E−31 | 0.312867678 | 0.523 | 0.389 | 7.96E−27 | A24 | Ssr1 |
| Nrp11 | 1.23E−30 | 0.329978654 | 0.426 | 0.278 | 3.05E−26 | A24 | Nrp1 |
| Ostc | 3.89E−30 | 0.308187678 | 0.514 | 0.373 | 9.65E−26 | A24 | Ostc |
| Kmo1 | 9.66E−30 | 0.268702408 | 0.778 | 0.756 | 2.40E−25 | A24 | Kmo |
| Ctnnb1 | 7.11E−29 | 0.328983549 | 0.566 | 0.457 | 1.77E−24 | A24 | Ctnnb1 |
| Uggt1 | 9.70E−29 | 0.280537946 | 0.385 | 0.245 | 2.41E−24 | A24 | Uggt1 |
| Gjb21 | 1.47E−28 | 0.29979701 | 0.709 | 0.635 | 3.66E−24 | A24 | Gjb2 |
| Uso1 | 2.20E−28 | 0.320233643 | 0.597 | 0.51 | 5.47E−24 | A24 | Uso1 |
| Slc3a1 | 2.88E−28 | 0.30743968 | 0.378 | 0.24 | 7.15E−24 | A24 | Slc3a1 |
| Efna1 | 5.06E−28 | 0.316132363 | 0.482 | 0.361 | 1.26E−23 | A24 | Efna1 |
| Usp9x | 1.17E−27 | 0.338687365 | 0.502 | 0.378 | 2.91E−23 | A24 | Usp9x |
| Sec61b1 | 2.12E−27 | 0.30077496 | 0.549 | 0.435 | 5.27E−23 | A24 | Sec61b |
| Calu | 4.28E−27 | 0.300472867 | 0.407 | 0.276 | 1.06E−22 | A24 | Calu |
| Ak3 | 4.61E−27 | 0.250448075 | 0.772 | 0.74 | 1.14E−22 | A24 | Ak3 |
| Nedd4 | 1.15E−26 | 0.330547905 | 0.647 | 0.583 | 2.86E−22 | A24 | Nedd4 |
| Usp4 | 5.04E−26 | 0.295902719 | 0.476 | 0.347 | 1.25E−21 | A24 | Usp4 |
| Gm54241 | 8.58E−26 | 0.2619882 | 0.445 | 0.309 | 2.13E−21 | A24 | Gm5424 |
| Dhcr24 | 1.11E−25 | 0.282375698 | 0.857 | 0.827 | 2.77E−21 | A24 | Dhcr24 |
| Rab1 | 1.32E−25 | 0.314483472 | 0.588 | 0.498 | 3.29E−21 | A24 | Rab1 |
| Eif4g11 | 3.05E−25 | 0.276286732 | 0.745 | 0.715 | 7.57E−21 | A24 | Eif4g1 |
| Acsl4 | 5.14E−25 | 0.299041198 | 0.478 | 0.365 | 1.28E−20 | A24 | Acsl4 |
| Dnajb11 | 1.54E−24 | 0.328784649 | 0.557 | 0.467 | 3.83E−20 | A24 | Dnajb11 |
| Srebf1 | 1.85E−24 | 0.314852133 | 0.439 | 0.319 | 4.59E−20 | A24 | Srebf1 |
| Arcn1 | 1.92E−24 | 0.304399062 | 0.53 | 0.421 | 4.76E−20 | A24 | Arcn1 |
| Copa | 6.77E−24 | 0.333406263 | 0.525 | 0.417 | 1.68E−19 | A24 | Copa |
| Mug21 | 1.27E−23 | 0.316623495 | 0.784 | 0.767 | 3.15E−19 | A24 | Mug2 |
| Il6st | 1.30E−23 | 0.301098452 | 0.448 | 0.329 | 3.23E−19 | A24 | Il6st |
| Etnk1 | 2.89E−23 | 0.319444078 | 0.49 | 0.384 | 7.17E−19 | A24 | Etnk1 |
| Prkd31 | 3.51E−23 | 0.304130117 | 0.631 | 0.557 | 8.72E−19 | A24 | Prkd3 |
| Rdx1 | 4.33E−23 | 0.276007288 | 0.729 | 0.695 | 1.07E−18 | A24 | Rdx |
| Ibtk | 5.93E−23 | 0.295901316 | 0.43 | 0.312 | 1.47E−18 | A24 | Ibtk |
| Lgr4 | 1.20E−22 | 0.257205387 | 0.399 | 0.279 | 2.99E−18 | A24 | Lgr4 |
| Srpr | 5.67E−22 | 0.291410089 | 0.532 | 0.442 | 1.41E−17 | A24 | Srpr |
| Lman11 | 6.76E−22 | 0.251809068 | 0.75 | 0.697 | 1.68E−17 | A24 | Lman1 |
| Edem1 | 1.41E−20 | 0.267537214 | 0.566 | 0.477 | 3.50E−16 | A24 | Edem1 |
| Pten | 1.56E−20 | 0.266292608 | 0.612 | 0.536 | 3.87E−16 | A24 | Pten |
| Lrrc591 | 5.25E−20 | 0.268768371 | 0.449 | 0.341 | 1.30E−15 | A24 | Lrrc59 |
| Srp72 | 6.96E−20 | 0.279992168 | 0.597 | 0.524 | 1.73E−15 | A24 | Srp72 |
| Nars1 | 1.10E−19 | 0.312705274 | 0.593 | 0.534 | 2.74E−15 | A24 | Nars |
| Ptprf1 | 1.19E−19 | 0.288833759 | 0.588 | 0.514 | 2.95E−15 | A24 | Ptprf |
| Trip12 | 1.43E−19 | 0.283180183 | 0.504 | 0.407 | 3.54E−15 | A24 | Trip12 |
| Mtus1 | 1.55E−19 | 0.312462778 | 0.563 | 0.485 | 3.85E−15 | A24 | Mtus1 |
| Stard4 | 2.95E−19 | 0.292186548 | 0.559 | 0.471 | 7.32E−15 | A24 | Stard4 |
| Proz | 3.55E−19 | 0.27996652 | 0.622 | 0.56 | 8.83E−15 | A24 | Proz |
| Txndc5 | 6.96E−19 | 0.275830553 | 0.48 | 0.378 | 1.73E−14 | A24 | Txndc5 |
| Echdc1 | 8.90E−19 | 0.281783333 | 0.503 | 0.423 | 2.21E−14 | A24 | Echdc1 |
| Gm242451 | 1.07E−18 | 0.41398979 | 0.369 | 0.262 | 2.65E−14 | A24 | Gm24245 |
| Sec31a | 1.72E−18 | 0.259599359 | 0.462 | 0.361 | 4.27E−14 | A24 | Sec31a |
| Sh3bgrl | 2.81E−18 | 0.26678946 | 0.512 | 0.42 | 6.99E−14 | A24 | Sh3bgrl |
| Mtss1 | 4.50E−18 | 0.252212375 | 0.581 | 0.508 | 1.12E−13 | A24 | Mtss1 |
| Sel1l | 7.10E−18 | 0.283345281 | 0.544 | 0.46 | 1.76E−13 | A24 | Sel1l |
| Ddost | 8.86E−18 | 0.290898225 | 0.493 | 0.411 | 2.20E−13 | A24 | Ddost |
| Slc38a10 | 2.68E−17 | 0.255657178 | 0.449 | 0.354 | 6.66E−13 | A24 | Slc38a10 |
| Nab1 | 5.44E−17 | 0.260054412 | 0.491 | 0.398 | 1.35E−12 | A24 | Nab1 |
| Nr5a2 | 9.20E−17 | 0.259898952 | 0.403 | 0.305 | 2.28E−12 | A24 | Nr5a2 |
| Met | 1.57E−16 | 0.251179491 | 0.434 | 0.344 | 3.91E−12 | A24 | Met |
| Stt3b | 1.78E−15 | 0.264816696 | 0.485 | 0.404 | 4.42E−11 | A24 | Stt3b |
| Atp11c | 2.49E−15 | 0.256760355 | 0.649 | 0.621 | 6.17E−11 | A24 | Atp11c |
| Lasp1 | 2.85E−15 | 0.2607968 | 0.361 | 0.268 | 7.09E−11 | A24 | Lasp1 |
| Grlf1 | 5.07E−15 | 0.255561206 | 0.413 | 0.321 | 1.26E−10 | A24 | Grlf1 |
| Fam120a | 2.45E−14 | 0.258279375 | 0.51 | 0.444 | 6.09E−10 | A24 | Fam120a |
| Eprs1 | 1.69E−12 | 0.251287379 | 0.587 | 0.553 | 4.20E−08 | A24 | Eprs |
| Lpgat11 | 2.69E−12 | 0.252503149 | 0.635 | 0.587 | 6.67E−08 | A24 | Lpgat1 |
| Zbtb201 | 5.86E−12 | 0.313482745 | 0.566 | 0.513 | 1.46E−07 | A24 | Zbtb20 |
| Fasn | 5.64E−09 | 0.269895808 | 0.449 | 0.394 | 0.000140063 | A24 | Fasn |
| Cyp4a142 | 0 | 1.280817508 | 0.991 | 0.5 | 0 | A48 | Cyp4a14 |
| Mat1a2 | 0 | 0.775338592 | 1 | 0.97 | 0 | A48 | Mat1a |
| Hmgcs22 | 0 | 0.768656201 | 1 | 0.977 | 0 | A48 | Hmgcs2 |
| Apoe2 | 1.02E−282 | 0.504851654 | 1 | 1 | 2.53E−278 | A48 | Apoe |
| Ces1c2 | 1.85E−278 | 0.548779002 | 0.999 | 0.984 | 4.59E−274 | A48 | Ces1c |
| Cyp4a101 | 2.26E−270 | 0.932584678 | 0.991 | 0.643 | 5.62E−266 | A48 | Cyp4a10 |
| Chchd102 | 1.01E−265 | 0.751681455 | 0.995 | 0.925 | 2.50E−261 | A48 | Chchd10 |
| Apoc21 | 4.27E−263 | 0.863852376 | 0.98 | 0.8 | 1.06E−258 | A48 | Apoc2 |
| Mfsd2a2 | 8.03E−247 | 0.661383645 | 0.731 | 0.203 | 1.99E−242 | A48 | Mfsd2a |
| Ttc362 | 5.87E−246 | 0.563789091 | 0.996 | 0.93 | 1.46E−241 | A48 | Ttc36 |
| Angptl32 | 1.51E−240 | 0.611080136 | 0.995 | 0.946 | 3.74E−236 | A48 | Angptl3 |
| Apoh2 | 2.05E−235 | 0.462791749 | 0.998 | 0.979 | 5.10E−231 | A48 | Apoh |
| Dbp | 5.52E−234 | 0.289556365 | 0.52 | 0.091 | 1.37E−229 | A48 | Dbp |
| Ahsg1 | 5.89E−227 | 0.359159618 | 1 | 0.999 | 1.46E−222 | A48 | Ahsg |
| Vtn1 | 2.03E−225 | 0.522813418 | 1 | 0.98 | 5.04E−221 | A48 | Vtn |
| Rgs16 | 5.76E−225 | 0.454464187 | 0.601 | 0.138 | 1.43E−220 | A48 | Rgs16 |
| Retsat2 | 2.28E−218 | 0.709570556 | 0.988 | 0.827 | 5.66E−214 | A48 | Retsat |
| Abcd2 | 2.96E−203 | 0.281300305 | 0.473 | 0.087 | 7.35E−199 | A48 | Abcd2 |
| Agt1 | 5.52E−203 | 0.57952745 | 0.981 | 0.814 | 1.37E−198 | A48 | Agt |
| Herpud11 | 1.06E−202 | 0.604376811 | 0.985 | 0.795 | 2.64E−198 | A48 | Herpud1 |
| Aldob | 1.64E−196 | 0.437497663 | 1 | 0.996 | 4.06E−192 | A48 | Aldob |
| Serping11 | 3.48E−195 | 0.577711276 | 0.987 | 0.863 | 8.63E−191 | A48 | Serping1 |
| Reep61 | 1.30E−193 | 0.623290477 | 0.967 | 0.791 | 3.24E−189 | A48 | Reep6 |
| Apoa51 | 1.41E−191 | 0.44834918 | 0.998 | 0.961 | 3.49E−187 | A48 | Apoa5 |
| Decr12 | 3.46E−191 | 0.566910147 | 0.96 | 0.678 | 8.60E−187 | A48 | Decr1 |
| Slc25a472 | 1.27E−188 | 0.61748351 | 0.992 | 0.917 | 3.16E−184 | A48 | Slc25a47 |
| F2 | 2.67E−188 | 0.395152093 | 0.999 | 0.969 | 6.63E−184 | A48 | F2 |
| Rdh72 | 6.84E−187 | 0.468441105 | 0.996 | 0.958 | 1.70E−182 | A48 | Rdh7 |
| Hrg1 | 3.57E−183 | 0.531568931 | 0.987 | 0.834 | 8.86E−179 | A48 | Hrg |
| Rbp41 | 4.56E−183 | 0.394209205 | 1 | 0.997 | 1.13E−178 | A48 | Rbp4 |
| Tef | 4.84E−177 | 0.339416398 | 0.651 | 0.192 | 1.20E−172 | A48 | Tef |
| Slco1a4 | 2.11E−176 | 0.509091975 | 0.77 | 0.291 | 5.23E−172 | A48 | Slco1a4 |
| Gc | 3.39E−175 | 0.297890227 | 1 | 0.998 | 8.41E−171 | A48 | Gc |
| Gm6484 | 3.94E−173 | 0.410774201 | 0.627 | 0.191 | 9.79E−169 | A48 | Gm6484 |
| Gm23935 | 1.27E−167 | 0.473837006 | 0.997 | 0.93 | 3.15E−163 | A48 | Gm23935 |
| Ugt2b52 | 3.54E−167 | 0.438817409 | 0.996 | 0.942 | 8.78E−163 | A48 | Ugt2b5 |
| Ttr2 | 2.12E−165 | 0.422488039 | 1 | 0.999 | 5.26E−161 | A48 | Ttr |
| Rpl38 | 1.26E−162 | 0.332203899 | 0.596 | 0.173 | 3.14E−158 | A48 | Rpl38 |
| St3gal52 | 9.27E−162 | 0.55492927 | 0.914 | 0.537 | 2.30E−157 | A48 | St3gal5 |
| Lcat1 | 4.41E−155 | 0.505352395 | 0.973 | 0.77 | 1.10E−150 | A48 | Lcat |
| Atp5h2 | 8.78E−153 | 0.597753903 | 0.933 | 0.64 | 2.18E−148 | A48 | Atp5h |
| Apoc32 | 4.46E−151 | 0.406980262 | 1 | 0.977 | 1.11E−146 | A48 | Apoc3 |
| Cp | 1.69E−147 | 0.427391341 | 0.995 | 0.947 | 4.21E−143 | A48 | Cp |
| Plin51 | 4.30E−146 | 0.398503015 | 0.788 | 0.332 | 1.07E−141 | A48 | Plin5 |
| Prodh21 | 2.43E−145 | 0.505471452 | 0.918 | 0.598 | 6.02E−141 | A48 | Prodh2 |
| Oaf1 | 3.43E−145 | 0.502082973 | 0.969 | 0.783 | 8.51E−141 | A48 | Oaf |
| Scand1 | 3.53E−145 | 0.270683065 | 0.579 | 0.175 | 8.77E−141 | A48 | Scand1 |
| Khk2 | 6.78E−145 | 0.520248448 | 0.982 | 0.855 | 1.68E−140 | A48 | Khk |
| Hmgcl2 | 1.35E−144 | 0.550084606 | 0.956 | 0.723 | 3.36E−140 | A48 | Hmgcl |
| Lims2 | 6.42E−144 | 0.376119911 | 0.679 | 0.253 | 1.59E−139 | A48 | Lims2 |
| Pnpla21 | 9.86E−144 | 0.346497418 | 0.687 | 0.253 | 2.45E−139 | A48 | Pnpla2 |
| Sult1a11 | 4.81E−142 | 0.482660126 | 0.963 | 0.718 | 1.20E−137 | A48 | Sult1a1 |
| Fth1 | 1.45E−141 | 0.37500962 | 0.996 | 0.975 | 3.60E−137 | A48 | Fth1 |
| Lpin11 | 9.79E−141 | 0.514964358 | 0.736 | 0.315 | 2.43E−136 | A48 | Lpin1 |
| Hdac111 | 4.34E−139 | 0.355729038 | 0.713 | 0.277 | 1.08E−134 | A48 | Hdac11 |
| Syvn1 | 4.46E−139 | 0.304118127 | 0.603 | 0.201 | 1.11E−134 | A48 | Syvn1 |
| Ttpa | 1.70E−138 | 0.443146694 | 0.991 | 0.876 | 4.23E−134 | A48 | Ttpa |
| Nr1i32 | 3.96E−138 | 0.47586273 | 0.814 | 0.371 | 9.85E−134 | A48 | Nr1i3 |
| Fkbp51 | 1.26E−136 | 0.257227973 | 0.54 | 0.158 | 3.12E−132 | A48 | Fkbp5 |
| Ephx21 | 6.88E−135 | 0.386872421 | 0.993 | 0.919 | 1.71E−130 | A48 | Ephx2 |
| Snord1181 | 9.71E−135 | 0.477121215 | 0.766 | 0.324 | 2.41E−130 | A48 | Snord118 |
| Slc27a51 | 3.81E−134 | 0.406176884 | 0.987 | 0.885 | 9.45E−130 | A48 | Slc27a5 |
| Ces1b | 1.19E−132 | 0.482951665 | 0.838 | 0.445 | 2.95E−128 | A48 | Ces1b |
| Ndrg21 | 1.81E−132 | 0.348203765 | 1 | 0.939 | 4.50E−128 | A48 | Ndrg2 |
| Cox8a2 | 3.15E−131 | 0.552001416 | 0.89 | 0.599 | 7.83E−127 | A48 | Cox8a |
| Cyp2a12 | 1.00E−129 | 0.466431941 | 0.981 | 0.879 | 2.49E−125 | A48 | Cyp2a12 |
| Cyp3a252 | 2.18E−129 | 0.360731032 | 0.995 | 0.902 | 5.41E−125 | A48 | Cyp3a25 |
| Ambp | 5.70E−128 | 0.285979982 | 1 | 0.991 | 1.42E−123 | A48 | Ambp |
| Pabpc1 | 5.17E−127 | 0.478200371 | 0.912 | 0.629 | 1.28E−122 | A48 | Pabpc1 |
| Mlycd1 | 9.54E−127 | 0.43784239 | 0.893 | 0.5 | 2.37E−122 | A48 | Mlycd |
| F101 | 1.50E−125 | 0.392724712 | 0.989 | 0.886 | 3.73E−121 | A48 | F10 |
| Gys21 | 2.76E−124 | 0.423060822 | 0.784 | 0.376 | 6.84E−120 | A48 | Gys2 |
| Cdo11 | 1.19E−123 | 0.397073051 | 0.997 | 0.956 | 2.96E−119 | A48 | Cdo1 |
| Echs11 | 2.01E−123 | 0.464869604 | 0.971 | 0.806 | 5.00E−119 | A48 | Echs1 |
| Bnip32 | 3.06E−123 | 0.462029744 | 0.876 | 0.529 | 7.61E−119 | A48 | Bnip3 |
| Slc37a41 | 4.81E−123 | 0.446122628 | 0.858 | 0.484 | 1.19E−118 | A48 | Slc37a4 |
| Tmbim61 | 1.33E−122 | 0.371305411 | 0.996 | 0.943 | 3.30E−118 | A48 | Tmbim6 |
| Bfar | 3.12E−122 | 0.308011346 | 0.742 | 0.306 | 7.75E−118 | A48 | Bfar |
| Ube2r21 | 4.52E−122 | 0.442298634 | 0.884 | 0.54 | 1.12E−117 | A48 | Ube2r2 |
| Abcb41 | 2.99E−121 | 0.442520105 | 0.956 | 0.688 | 7.42E−117 | A48 | Abcb4 |
| Grn1 | 6.37E−120 | 0.431036339 | 0.844 | 0.449 | 1.58E−115 | A48 | Grn |
| Slc25a102 | 3.22E−119 | 0.431271222 | 0.877 | 0.526 | 8.00E−115 | A48 | Slc25a10 |
| Hpx1 | 7.59E−118 | 0.385111507 | 0.999 | 0.997 | 1.89E−113 | A48 | Hpx |
| Vnn3 | 4.99E−116 | 0.300835625 | 0.684 | 0.275 | 1.24E−111 | A48 | Vnn3 |
| Fcgrt1 | 2.63E−115 | 0.408442054 | 0.91 | 0.576 | 6.53E−111 | A48 | Fcgrt |
| Fah1 | 8.72E−115 | 0.391286468 | 0.974 | 0.821 | 2.17E−110 | A48 | Fah |
| Atp5b | 1.69E−114 | 0.328113534 | 0.997 | 0.94 | 4.19E−110 | A48 | Atp5b |
| Amy12 | 4.53E−114 | 0.421579258 | 0.943 | 0.66 | 1.13E−109 | A48 | Amy1 |
| Gstt3 | 3.70E−113 | 0.338366426 | 0.737 | 0.312 | 9.19E−109 | A48 | Gstt3 |
| Ech11 | 1.29E−112 | 0.418013348 | 0.98 | 0.828 | 3.21E−108 | A48 | Ech1 |
| Hsd17b131 | 1.32E−112 | 0.622071742 | 0.934 | 0.741 | 3.29E−108 | A48 | Hsd17b13 |
| Serpina3m1 | 6.99E−112 | 0.40339321 | 0.949 | 0.749 | 1.74E−107 | A48 | Serpina3m |
| Serpina1d2 | 2.32E−111 | 0.30529594 | 0.997 | 0.977 | 5.77E−107 | A48 | Serpina1d |
| Decr21 | 2.53E−109 | 0.412881852 | 0.92 | 0.644 | 6.29E−105 | A48 | Decr2 |
| Hspa82 | 5.67E−109 | 0.410735253 | 0.952 | 0.731 | 1.41E−104 | A48 | Hspa8 |
| Gltscr2 | 1.30E−108 | 0.343207156 | 0.83 | 0.435 | 3.24E−104 | A48 | Gltscr2 |
| Slc25a20 | 2.03E−108 | 0.387540813 | 0.877 | 0.508 | 5.03E−104 | A48 | Slc25a20 |
| Abhd14b1 | 5.35E−108 | 0.405910897 | 0.885 | 0.554 | 1.33E−103 | A48 | Abhd14b |
| Cebpb2 | 2.85E−107 | 0.368375731 | 0.815 | 0.414 | 7.09E−103 | A48 | Cebpb |
| Ahcy1 | 6.37E−107 | 0.339983635 | 0.988 | 0.875 | 1.58E−102 | A48 | Ahcy |
| F12 | 9.50E−107 | 0.388008981 | 0.966 | 0.803 | 2.36E−102 | A48 | F12 |
| Cox5a1 | 2.15E−106 | 0.41972236 | 0.938 | 0.693 | 5.34E−102 | A48 | Cox5a |
| Tcea31 | 3.05E−106 | 0.39376107 | 0.825 | 0.448 | 7.58E−102 | A48 | Tcea3 |
| Tmbim41 | 6.73E−106 | 0.369287383 | 0.784 | 0.407 | 1.67E−101 | A48 | Tmbim4 |
| Acad11 | 1.16E−105 | 0.38508652 | 0.896 | 0.583 | 2.88E−101 | A48 | Acad11 |
| Gabarap1 | 8.50E−105 | 0.40143345 | 0.956 | 0.75 | 2.11E−100 | A48 | Gabarap |
| Aldh9a1 | 9.77E−105 | 0.382553818 | 0.964 | 0.772 | 2.43E−100 | A48 | Aldh9a1 |
| Ddb1 | 2.16E−103 | 0.354415928 | 0.83 | 0.452 | 5.37E−99 | A48 | Ddb1 |
| P4hb2 | 5.46E−103 | 0.298062011 | 0.995 | 0.942 | 1.36E−98 | A48 | P4hb |
| Yipf3 | 1.81E−102 | 0.278463646 | 0.68 | 0.291 | 4.50E−98 | A48 | Yipf3 |
| Sh3glb1 | 9.56E−102 | 0.304360744 | 0.695 | 0.306 | 2.37E−97 | A48 | Sh3glb1 |
| Rpl4 | 2.39E−101 | 0.328862408 | 0.987 | 0.912 | 5.93E−97 | A48 | Rpl4 |
| Ctsh1 | 2.95E−101 | 0.422416234 | 0.945 | 0.72 | 7.32E−97 | A48 | Ctsh |
| Apoa41 | 1.97E−100 | 0.494713556 | 0.778 | 0.4 | 4.90E−96 | A48 | Apoa4 |
| Mgll | 3.72E−100 | 0.365109404 | 0.844 | 0.477 | 9.23E−96 | A48 | Mgll |
| Ppara1 | 6.73E−100 | 0.339317968 | 0.831 | 0.443 | 1.67E−95 | A48 | Ppara |
| 1600002H07Rik | 1.17E−98 | 0.30157504 | 0.736 | 0.353 | 2.91E−94 | A48 | 1600002H07Rik |
| G0s21 | 1.31E−98 | 0.387970035 | 0.692 | 0.32 | 3.26E−94 | A48 | G0s2 |
| Cox6a12 | 1.81E−98 | 0.371822664 | 0.987 | 0.854 | 4.49E−94 | A48 | Cox6a1 |
| Igfbp4 | 6.46E−98 | 0.339536078 | 0.99 | 0.875 | 1.60E−93 | A48 | Igfbp4 |
| C4b1 | 2.60E−97 | 0.398907532 | 0.975 | 0.876 | 6.46E−93 | A48 | C4b |
| Sdhc | 2.46E−96 | 0.38650315 | 0.91 | 0.645 | 6.10E−92 | A48 | Sdhc |
| Apoc42 | 3.94E−96 | 0.409636949 | 0.998 | 0.965 | 9.79E−92 | A48 | Apoc4 |
| Serpinf1 | 4.30E−96 | 0.356520958 | 0.976 | 0.855 | 1.07E−91 | A48 | Serpinf1 |
| Wbp1l1 | 7.44E−96 | 0.38750996 | 0.91 | 0.648 | 1.85E−91 | A48 | Wbp1l |
| Aldoa | 1.89E−95 | 0.277740845 | 0.719 | 0.329 | 4.70E−91 | A48 | Aldoa |
| Atp5k1 | 2.67E−95 | 0.384272907 | 0.935 | 0.664 | 6.63E−91 | A48 | Atp5k |
| Aspdh | 5.69E−95 | 0.321708599 | 0.776 | 0.397 | 1.41E−90 | A48 | Aspdh |
| Gm2a | 4.55E−94 | 0.349188726 | 0.827 | 0.459 | 1.13E−89 | A48 | Gm2a |
| Etnppl2 | 1.17E−93 | 0.525305142 | 0.866 | 0.587 | 2.89E−89 | A48 | Etnppl |
| Etfb1 | 8.54E−93 | 0.309173682 | 0.996 | 0.928 | 2.12E−88 | A48 | Etfb |
| Sec14l21 | 1.30E−92 | 0.352363823 | 0.98 | 0.835 | 3.23E−88 | A48 | Sec14l2 |
| Camk2n11 | 2.32E−92 | 0.273006774 | 0.691 | 0.315 | 5.77E−88 | A48 | Camk2n1 |
| Slc10a11 | 2.86E−92 | 0.375331232 | 0.984 | 0.84 | 7.10E−88 | A48 | Slc10a1 |
| Ifnar2 | 6.13E−92 | 0.274914839 | 0.731 | 0.34 | 1.52E−87 | A48 | Ifnar2 |
| Cpt2 | 8.07E−92 | 0.326691619 | 0.807 | 0.437 | 2.00E−87 | A48 | Cpt2 |
| Azgp1 | 2.23E−91 | 0.252008502 | 0.997 | 0.966 | 5.53E−87 | A48 | Azgp1 |
| Uroc1 | 1.35E−90 | 0.407426649 | 0.914 | 0.637 | 3.34E−86 | A48 | Uroc1 |
| Pbld11 | 6.93E−90 | 0.345326717 | 0.881 | 0.566 | 1.72E−85 | A48 | Pbld1 |
| Brap | 1.06E−89 | 0.315930007 | 0.829 | 0.465 | 2.62E−85 | A48 | Brap |
| Shmt11 | 1.20E−89 | 0.347962893 | 0.882 | 0.556 | 2.98E−85 | A48 | Shmt1 |
| Slc27a2 | 1.39E−89 | 0.313906428 | 0.996 | 0.945 | 3.44E−85 | A48 | Slc27a2 |
| Asgr21 | 7.88E−89 | 0.348507534 | 0.879 | 0.552 | 1.96E−84 | A48 | Asgr2 |
| Aco2 | 3.32E−88 | 0.326709931 | 0.846 | 0.507 | 8.24E−84 | A48 | Aco2 |
| Ctsd | 1.28E−87 | 0.354466764 | 0.916 | 0.642 | 3.17E−83 | A48 | Ctsd |
| Cyp3a112 | 3.21E−87 | 0.397040898 | 1 | 0.995 | 7.98E−83 | A48 | Cyp3a11 |
| Sdhd2 | 4.40E−87 | 0.434718095 | 0.891 | 0.655 | 1.09E−82 | A48 | Sdhd |
| 0610005C13Rik1 | 6.63E−86 | 0.335559673 | 0.944 | 0.665 | 1.65E−81 | A48 | 0610005C13Rik |
| Slc40a11 | 9.34E−86 | 0.272553886 | 0.744 | 0.367 | 2.32E−81 | A48 | Slc40a1 |
| Cyp2c441 | 1.12E−85 | 0.414935231 | 0.924 | 0.692 | 2.77E−81 | A48 | Cyp2c44 |
| Lamp11 | 1.61E−85 | 0.327385277 | 0.982 | 0.877 | 4.01E−81 | A48 | Lamp1 |
| Creb3l3 | 5.60E−85 | 0.330494287 | 0.971 | 0.818 | 1.39E−80 | A48 | Creb3l3 |
| Pigr | 1.16E−84 | 0.40014252 | 0.977 | 0.945 | 2.89E−80 | A48 | Pigr |
| Apoc12 | 4.87E−84 | 0.292819411 | 1 | 0.992 | 1.21E−79 | A48 | Apoc1 |
| Smlr11 | 5.67E−84 | 0.251582244 | 0.68 | 0.317 | 1.41E−79 | A48 | Sm1r1 |
| Snx3 | 7.63E−84 | 0.256302059 | 0.693 | 0.331 | 1.89E−79 | A48 | Snx3 |
| Atp5g31 | 1.55E−83 | 0.328843609 | 0.985 | 0.888 | 3.85E−79 | A48 | Atp5g3 |
| Cyp2d26 | 4.14E−83 | 0.305047306 | 0.982 | 0.897 | 1.03E−78 | A48 | Cyp2d26 |
| Klf9 | 4.69E−83 | 0.332611254 | 0.862 | 0.549 | 1.17E−78 | A48 | Klf9 |
| Eci2 | 5.28E−83 | 0.328111149 | 0.944 | 0.697 | 1.31E−78 | A48 | Eci2 |
| Slc2a21 | 7.42E−83 | 0.356962096 | 0.824 | 0.485 | 1.84E−78 | A48 | Slc2a2 |
| Acot12 | 6.96E−82 | 0.278479827 | 0.825 | 0.454 | 1.73E−77 | A48 | Acot12 |
| Tmem120a | 1.04E−81 | 0.252496012 | 0.708 | 0.343 | 2.59E−77 | A48 | Tmem120a |
| Pglyrp2 | 6.41E−81 | 0.307416307 | 0.754 | 0.402 | 1.59E−76 | A48 | Pglyrp2 |
| Abcc3 | 1.62E−80 | 0.35576814 | 0.859 | 0.549 | 4.03E−76 | A48 | Abcc3 |
| Fam195a1 | 4.52E−80 | 0.286110606 | 0.726 | 0.359 | 1.12E−75 | A48 | Fam195a |
| Ctsa | 8.45E−80 | 0.25495645 | 0.731 | 0.36 | 2.10E−75 | A48 | Ctsa |
| Oplah | 1.28E−79 | 0.303362318 | 0.805 | 0.457 | 3.19E−75 | A48 | Oplah |
| Acat11 | 4.32E−79 | 0.307325333 | 0.978 | 0.842 | 1.07E−74 | A48 | Acat1 |
| Park71 | 3.18E−78 | 0.343705589 | 0.953 | 0.774 | 7.89E−74 | A48 | Park7 |
| Pbld2 | 4.10E−78 | 0.312075493 | 0.891 | 0.58 | 1.02E−73 | A48 | Pbld2 |
| Serpina3n1 | 9.63E−78 | 0.318139347 | 0.902 | 0.626 | 2.39E−73 | A48 | Serpina3n |
| Serinc31 | 3.42E−77 | 0.289735353 | 0.793 | 0.452 | 8.48E−73 | A48 | Serinc3 |
| Pitpnb | 8.76E−77 | 0.266116041 | 0.795 | 0.431 | 2.18E−72 | A48 | Pitpnb |
| Usmg52 | 9.41E−77 | 0.376132402 | 0.755 | 0.419 | 2.34E−72 | A48 | Usmg5 |
| Ninj11 | 9.74E−77 | 0.346226664 | 0.874 | 0.587 | 2.42E−72 | A48 | Ninj1 |
| Hsd17b102 | 2.43E−76 | 0.404734527 | 0.927 | 0.686 | 6.03E−72 | A48 | Hsd17b10 |
| Eef1d | 6.80E−75 | 0.257204457 | 0.795 | 0.434 | 1.69E−70 | A48 | Eef1d |
| Lipa1 | 2.40E−74 | 0.31862164 | 0.904 | 0.63 | 5.96E−70 | A48 | Lipa |
| Agpat6 | 3.14E−74 | 0.289480086 | 0.884 | 0.558 | 7.81E−70 | A48 | Agpat6 |
| Pim31 | 3.82E−74 | 0.313735047 | 0.845 | 0.52 | 9.48E−70 | A48 | Pim3 |
| Pcx | 5.84E−74 | 0.315978553 | 0.912 | 0.66 | 1.45E−69 | A48 | Pcx |
| Creg11 | 3.96E−73 | 0.2979552 | 0.993 | 0.917 | 9.83E−69 | A48 | Creg1 |
| Gcdh1 | 4.36E−73 | 0.288485226 | 0.977 | 0.848 | 1.08E−68 | A48 | Gcdh |
| Atp5d1 | 1.54E−72 | 0.307608726 | 0.935 | 0.694 | 3.81E−68 | A48 | Atp5d |
| Gfra11 | 1.91E−72 | 0.288958416 | 0.867 | 0.539 | 4.74E−68 | A48 | Gfra1 |
| Pck11 | 2.03E−72 | 0.350075486 | 0.999 | 0.974 | 5.03E−68 | A48 | Pck1 |
| Gstt2 | 1.93E−71 | 0.25926769 | 0.754 | 0.398 | 4.80E−67 | A48 | Gstt2 |
| Sord1 | 3.59E−71 | 0.301872945 | 0.987 | 0.917 | 8.92E−67 | A48 | Sord |
| Tmem371 | 7.54E−71 | 0.261063123 | 0.786 | 0.429 | 1.87E−66 | A48 | Tmem37 |
| Pla2g12b | 8.56E−71 | 0.27882702 | 0.798 | 0.457 | 2.13E−66 | A48 | Pla2g12b |
| Ndufb22 | 1.82E−70 | 0.348400962 | 0.888 | 0.589 | 4.53E−66 | A48 | Ndufb2 |
| Atf5 | 2.73E−70 | 0.317193075 | 0.909 | 0.649 | 6.78E−66 | A48 | Atf5 |
| Sfxn1 | 2.87E−70 | 0.329269537 | 0.822 | 0.522 | 7.13E−66 | A48 | Sfxn1 |
| Glrx52 | 3.18E−70 | 0.299962013 | 0.886 | 0.568 | 7.89E−66 | A48 | Glrx5 |
| Atp5j1 | 2.90E−69 | 0.350683282 | 0.916 | 0.73 | 7.19E−65 | A48 | Atp5j |
| Ggnbp2 | 3.08E−69 | 0.257783021 | 0.818 | 0.458 | 7.65E−65 | A48 | Ggnbp2 |
| Furin | 4.55E−69 | 0.280556705 | 0.781 | 0.445 | 1.13E−64 | A48 | Furin |
| Ndufb101 | 7.41E−69 | 0.316418226 | 0.907 | 0.669 | 1.84E−64 | A48 | Ndufb10 |
| Dhrs41 | 1.15E−68 | 0.276531082 | 0.94 | 0.671 | 2.87E−64 | A48 | Dhrs4 |
| Sec61a1 | 1.50E−68 | 0.289912357 | 0.825 | 0.513 | 3.72E−64 | A48 | Sec61a1 |
| Zfp91 | 1.51E−68 | 0.275372132 | 0.855 | 0.531 | 3.75E−64 | A48 | Zfp91 |
| D17Wsu92e | 1.81E−68 | 0.282647467 | 0.909 | 0.62 | 4.49E−64 | A48 | D17Wsu92e |
| Sult1d12 | 2.07E−68 | 0.43989704 | 0.936 | 0.68 | 5.14E−64 | A48 | Sult1d1 |
| Mst1 | 3.99E−68 | 0.277541098 | 0.81 | 0.489 | 9.91E−64 | A48 | Mst1 |
| Igf1 | 7.06E−68 | 0.302677789 | 0.985 | 0.902 | 1.75E−63 | A48 | Igf1 |
| Acadvl | 8.03E−68 | 0.280297075 | 0.959 | 0.782 | 1.99E−63 | A48 | Acadvl |
| Hgfac | 1.41E−67 | 0.265617858 | 0.786 | 0.457 | 3.49E−63 | A48 | Hgfac |
| 11-Mar | 1.82E−67 | 0.28124308 | 0.984 | 0.869 | 4.51E−63 | A48 | 1-Mar |
| Tgoln1 | 4.48E−67 | 0.305516812 | 0.891 | 0.602 | 1.11E−62 | A48 | Tgoln1 |
| Upb11 | 4.61E−67 | 0.305876376 | 0.945 | 0.746 | 1.14E−62 | A48 | Upb1 |
| Acbd51 | 7.35E−67 | 0.284090869 | 0.943 | 0.711 | 1.82E−62 | A48 | Acbd5 |
| Sds2 | 2.26E−66 | 0.553946754 | 0.799 | 0.612 | 5.62E−62 | A48 | Sds |
| Map2k2 | 2.40E−66 | 0.255067193 | 0.832 | 0.517 | 5.96E−62 | A48 | Map2k2 |
| Ndufs71 | 2.84E−66 | 0.254510701 | 0.847 | 0.51 | 7.05E−62 | A48 | Ndufs7 |
| Dcxr2 | 3.22E−66 | 0.298871488 | 0.884 | 0.591 | 7.99E−62 | A48 | Dcxr |
| Gpt1 | 3.35E−66 | 0.288097988 | 0.94 | 0.737 | 8.32E−62 | A48 | Gpt |
| Gpld1 | 6.68E−66 | 0.288714573 | 0.877 | 0.595 | 1.66E−61 | A48 | Gpld1 |
| 1100001G20Rik1 | 7.71E−66 | 0.341544398 | 0.993 | 0.968 | 1.91E−61 | A48 | 110000 1G20Rik |
| Ndufs81 | 2.39E−65 | 0.314878255 | 0.854 | 0.581 | 5.94E−61 | A48 | Ndufs8 |
| Abca6 | 5.35E−65 | 0.284030069 | 0.849 | 0.548 | 1.33E−60 | A48 | Abca6 |
| Actn4 | 4.59E−64 | 0.25335849 | 0.789 | 0.465 | 1.14E−59 | A48 | Actn4 |
| Gchfr2 | 2.18E−63 | 0.323950621 | 0.953 | 0.727 | 5.42E−59 | A48 | Gchfr |
| Mia3 | 6.64E−63 | 0.284751778 | 0.91 | 0.64 | 1.65E−58 | A48 | Mia3 |
| Cd1d11 | 8.52E−63 | 0.292681939 | 0.887 | 0.611 | 2.11E−58 | A48 | Cd1d1 |
| Tmem176b1 | 9.86E−63 | 0.288247134 | 0.89 | 0.63 | 2.45E−58 | A48 | Tmem176b |
| Lpin21 | 1.01E−62 | 0.304127419 | 0.921 | 0.653 | 2.52E−58 | A48 | Lpin2 |
| Pccb | 1.17E−62 | 0.280165918 | 0.901 | 0.642 | 2.90E−58 | A48 | Pccb |
| Cideb2 | 1.52E−62 | 0.315951718 | 0.892 | 0.65 | 3.79E−58 | A48 | Cideb |
| Itih1 | 2.19E−62 | 0.288782591 | 0.974 | 0.819 | 5.45E−58 | A48 | Itih1 |
| B2m2 | 2.44E−62 | 0.257225676 | 0.997 | 0.949 | 6.06E−58 | A48 | B2m |
| Maob | 5.02E−62 | 0.288484521 | 0.889 | 0.597 | 1.25E−57 | A48 | Maob |
| Slc25a39 | 6.32E−62 | 0.295889572 | 0.938 | 0.762 | 1.57E−57 | A48 | Slc25a39 |
| Acadl | 9.46E−62 | 0.260302089 | 0.916 | 0.643 | 2.35E−57 | A48 | Acadl |
| Hadh1 | 1.41E−61 | 0.279987697 | 0.908 | 0.641 | 3.49E−57 | A48 | Hadh |
| Akr1c191 | 5.83E−61 | 0.295184218 | 0.821 | 0.507 | 1.45E−56 | A48 | Akr1c19 |
| Os9 | 1.17E−60 | 0.268248599 | 0.911 | 0.673 | 2.90E−56 | A48 | Os9 |
| Pemt1 | 1.39E−60 | 0.316750177 | 0.948 | 0.754 | 3.46E−56 | A48 | Pemt |
| Hsbp11 | 1.15E−59 | 0.2547665 | 0.792 | 0.495 | 2.86E−55 | A48 | Hsbp1 |
| Serpinf2 | 2.36E−59 | 0.262659763 | 0.966 | 0.847 | 5.86E−55 | A48 | Serpinf2 |
| Ndufa131 | 6.13E−59 | 0.286435118 | 0.959 | 0.769 | 1.52E−54 | A48 | Ndufa13 |
| Gsto1 | 6.52E−59 | 0.279937059 | 0.818 | 0.518 | 1.62E−54 | A48 | Gsto1 |
| Ubxn4 | 2.22E−58 | 0.25604972 | 0.893 | 0.601 | 5.52E−54 | A48 | Ubxn4 |
| Slc25a13 | 2.47E−58 | 0.263619116 | 0.924 | 0.668 | 6.14E−54 | A48 | Slc25a13 |
| Hadha | 3.06E−58 | 0.279003545 | 0.943 | 0.743 | 7.60E−54 | A48 | Hadha |
| Surf4 | 3.88E−58 | 0.252260406 | 0.873 | 0.578 | 9.64E−54 | A48 | Surf4 |
| Hal1 | 4.19E−58 | 0.382660599 | 0.908 | 0.741 | 1.04E−53 | A48 | Ha1 |
| Akr1d11 | 1.29E−57 | 0.264802354 | 0.852 | 0.543 | 3.22E−53 | A48 | Akr1d1 |
| Rabac12 | 5.41E−56 | 0.250868483 | 0.807 | 0.498 | 1.34E−51 | A48 | Rabac1 |
| Tspo1 | 7.02E−56 | 0.266009062 | 0.784 | 0.477 | 1.74E−51 | A48 | Tspo |
| Mttp | 9.90E−56 | 0.261697738 | 0.933 | 0.709 | 2.46E−51 | A48 | Mttp |
| Ctage5 | 2.71E−55 | 0.27450678 | 0.941 | 0.698 | 6.74E−51 | A48 | Ctage5 |
| Agpat3 | 3.06E−55 | 0.25259632 | 0.874 | 0.617 | 7.61E−51 | A48 | Agpat3 |
| Gamt1 | 3.37E−55 | 0.268064254 | 0.841 | 0.564 | 8.36E−51 | A48 | Gamt |
| Dgat21 | 3.63E−55 | 0.26370048 | 0.947 | 0.753 | 9.02E−51 | A48 | Dgat2 |
| Sardh | 7.35E−53 | 0.278384633 | 0.959 | 0.829 | 1.82E−48 | A48 | Sardh |
| Asl1 | 2.33E−51 | 0.336107029 | 0.953 | 0.801 | 5.79E−47 | A48 | Asl |
| Acsl12 | 5.59E−51 | 0.254529662 | 0.978 | 0.895 | 1.39E−46 | A48 | Acsl1 |
| Ccs1 | 9.34E−50 | 0.264321005 | 0.895 | 0.655 | 2.32E−45 | A48 | Ccs |
| BC089597 | 1.42E−49 | 0.250990415 | 0.813 | 0.516 | 3.52E−45 | A48 | BC089597 |
| Por2 | 5.03E−49 | 0.289329631 | 0.921 | 0.688 | 1.25E−44 | A48 | Por |
| Uqcrfs1 | 6.79E−48 | 0.257925751 | 0.949 | 0.781 | 1.69E−43 | A48 | Uqcrfs1 |
| Hagh1 | 4.03E−47 | 0.287377171 | 0.959 | 0.834 | 1.00E−42 | A48 | Hagh |
| Tmem14c1 | 5.76E−47 | 0.250667772 | 0.837 | 0.561 | 1.43E−42 | A48 | Tmem14c |
| Dak | 5.90E−47 | 0.296630929 | 0.852 | 0.575 | 1.47E−42 | A48 | Dak |
| Leap22 | 2.60E−44 | 0.335628781 | 0.833 | 0.576 | 6.45E−40 | A48 | Leap2 |
| Cpt1a | 1.94E−43 | 0.264989382 | 0.896 | 0.681 | 4.81E−39 | A48 | Cpt1a |
| Mpc22 | 1.37E−42 | 0.287450301 | 0.953 | 0.826 | 3.40E−38 | A48 | Mpc2 |
| Cd3021 | 6.25E−40 | 0.271235165 | 0.956 | 0.844 | 1.55E−35 | A48 | Cd302 |
| Rps27l2 | 8.74E−39 | 0.286856723 | 0.924 | 0.795 | 2.17E−34 | A48 | Rps27l |
| Cyp2f21 | 3.99E−31 | 0.296079879 | 0.912 | 0.833 | 9.91E−27 | A48 | Cyp2f2 |
| Slc7a2 | 3.98E−30 | 0.250213104 | 0.921 | 0.758 | 9.87E−26 | A48 | Slc7a2 |
| Glul | 2.05E−13 | 0.540145967 | 0.308 | 0.18 | 5.09E−09 | A48 | Glul |
| Apoc13 | 1.14E−259 | 0.549525053 | 1 | 0.992 | 2.84E−255 | A96 | Apoc1 |
| Cyp4a143 | 2.66E−250 | 0.970243185 | 0.975 | 0.511 | 6.60E−246 | A96 | Cyp4a14 |
| Cyp4a102 | 2.15E−216 | 0.868151957 | 0.974 | 0.651 | 5.34E−212 | A96 | Cyp4a10 |
| Mat1a3 | 3.10E−210 | 0.596679364 | 1 | 0.97 | 7.70E−206 | A96 | Mat1a |
| Angptl33 | 5.10E−206 | 0.560557268 | 0.998 | 0.946 | 1.27E−201 | A96 | Angptl3 |
| Cdo12 | 3.07E−182 | 0.508585858 | 0.998 | 0.957 | 7.61E−178 | A96 | Cdo1 |
| Apoa22 | 2.85E−177 | 0.344745894 | 1 | 0.999 | 7.07E−173 | A96 | Apoa2 |
| Zfp683 | 2.01E−176 | 0.41843816 | 0.476 | 0.092 | 4.99E−172 | A96 | Zfp683 |
| Apoe3 | 2.87E−148 | 0.316142135 | 1 | 1 | 7.13E−144 | A96 | Apoe |
| Chchd103 | 1.38E−138 | 0.47683652 | 0.994 | 0.927 | 3.43E−134 | A96 | Chchd10 |
| Ttr3 | 2.28E−137 | 0.317447356 | 1 | 0.999 | 5.66E−133 | A96 | Ttr |
| Ttpa1 | 4.82E−129 | 0.492631097 | 0.98 | 0.879 | 1.20E−124 | A96 | Ttpa |
| Scd12 | 2.34E−118 | 0.520262727 | 0.989 | 0.926 | 5.82E−114 | A96 | Scd1 |
| Ubc2 | 2.86E−109 | 0.480972081 | 0.968 | 0.839 | 7.10E−105 | A96 | Ubc |
| Apoh3 | 2.32E−107 | 0.308461461 | 0.998 | 0.979 | 5.76E−103 | A96 | Apoh |
| Ces1d1 | 9.98E−105 | 0.450848726 | 0.977 | 0.905 | 2.48E−100 | A96 | Ces1d |
| Apoc33 | 1.84E−102 | 0.338145432 | 1 | 0.978 | 4.56E−98 | A96 | Apoc3 |
| Ces1c3 | 8.24E−97 | 0.298285129 | 1 | 0.985 | 2.05E−92 | A96 | Ces1c |
| Hmgcs23 | 2.29E−95 | 0.304092138 | 0.996 | 0.977 | 5.70E−91 | A96 | Hmgcs2 |
| Slco1b21 | 3.01E−92 | 0.515680504 | 0.998 | 0.929 | 7.48E−88 | A96 | Slco1b2 |
| Cyp8b12 | 6.15E−88 | 0.516691772 | 0.897 | 0.691 | 1.53E−83 | A96 | Cyp8b1 |
| Hamp2 | 4.13E−86 | 0.548864769 | 0.92 | 0.788 | 1.03E−81 | A96 | Hamp |
| Slc27a21 | 7.10E−80 | 0.325141901 | 0.988 | 0.947 | 1.76E−75 | A96 | Slc27a2 |
| Hspa83 | 1.35E−77 | 0.414165913 | 0.905 | 0.741 | 3.34E−73 | A96 | Hspa8 |
| Mettl7b1 | 3.18E−76 | 0.370125266 | 0.949 | 0.866 | 7.90E−72 | A96 | Mettl7b |
| Cyp3a113 | 1.94E−75 | 0.275004032 | 0.999 | 0.995 | 4.82E−71 | A96 | Cyp3a11 |
| Crot1 | 7.79E−75 | 0.314330235 | 0.989 | 0.942 | 1.94E−70 | A96 | Crot |
| Cyp2c292 | 1.44E−68 | 0.551021967 | 0.962 | 0.85 | 3.59E−64 | A96 | Cyp2c29 |
| Acbd52 | 3.67E−64 | 0.390303062 | 0.87 | 0.725 | 9.12E−60 | A96 | Acbd5 |
| Acsl13 | 2.22E−63 | 0.329698108 | 0.977 | 0.897 | 5.52E−59 | A96 | Acsl1 |
| Sult1a12 | 2.88E−62 | 0.384980426 | 0.881 | 0.733 | 7.15E−58 | A96 | Sult1a1 |
| Aldh2 | 6.54E−62 | 0.2825177 | 0.995 | 0.968 | 1.62E−57 | A96 | Aldh2 |
| Retsat3 | 2.39E−61 | 0.365551797 | 0.957 | 0.834 | 5.92E−57 | A96 | Retsat |
| Abcb42 | 3.19E−61 | 0.389954192 | 0.878 | 0.704 | 7.92E−57 | A96 | Abcb4 |
| Ndrg22 | 1.13E−60 | 0.2700141 | 0.985 | 0.942 | 2.81E−56 | A96 | Ndrg2 |
| Aldh3a22 | 3.23E−60 | 0.565894922 | 0.687 | 0.451 | 8.02E−56 | A96 | Aldh3a2 |
| Atp5k2 | 9.30E−60 | 0.371467827 | 0.837 | 0.682 | 2.31E−55 | A96 | Atp5k |
| Amy13 | 2.54E−58 | 0.386150545 | 0.831 | 0.681 | 6.31E−54 | A96 | Amy1 |
| Igfbp41 | 2.23E−56 | 0.295776474 | 0.964 | 0.881 | 5.53E−52 | A96 | Igfbp4 |
| Chpt11 | 2.52E−56 | 0.380201964 | 0.848 | 0.694 | 6.27E−52 | A96 | Chpt1 |
| Pex11a1 | 4.35E−56 | 0.446250068 | 0.7 | 0.484 | 1.08E−51 | A96 | Pex11a |
| Hhex1 | 7.20E−54 | 0.355789479 | 0.543 | 0.313 | 1.79E−49 | A96 | Hhex |
| Sult1d13 | 2.09E−51 | 0.389921134 | 0.855 | 0.696 | 5.20E−47 | A96 | Sult1d1 |
| Atp5h3 | 1.30E−50 | 0.314445524 | 0.848 | 0.657 | 3.24E−46 | A96 | Atp5h |
| Cyp3a253 | 5.89E−50 | 0.2702962 | 0.981 | 0.905 | 1.46E−45 | A96 | Cyp3a25 |
| Lipa2 | 1.77E−49 | 0.355944215 | 0.791 | 0.651 | 4.41E−45 | A96 | Lipa |
| Cyp2e12 | 4.42E−49 | 0.511826271 | 0.93 | 0.809 | 1.10E−44 | A96 | Cyp2e1 |
| Prlr | 2.34E−48 | 0.346406091 | 0.574 | 0.359 | 5.82E−44 | A96 | Prlr |
| Clpx1 | 3.44E−48 | 0.325403599 | 0.884 | 0.764 | 8.54E−44 | A96 | Clpx |
| Tmem56 | 4.52E−47 | 0.334247655 | 0.812 | 0.643 | 1.12E−42 | A96 | Tmem56 |
| Insig22 | 1.46E−46 | 0.285386985 | 0.928 | 0.818 | 3.63E−42 | A96 | Insig2 |
| Hrg2 | 6.03E−46 | 0.280535908 | 0.923 | 0.845 | 1.50E−41 | A96 | Hrg |
| Ube2r22 | 1.09E−45 | 0.338184842 | 0.747 | 0.565 | 2.70E−41 | A96 | Ube2r2 |
| Ppp2r5a1 | 3.21E−45 | 0.350536833 | 0.764 | 0.615 | 7.96E−41 | A96 | Ppp2r5a |
| Nr1i33 | 6.04E−44 | 0.348125519 | 0.625 | 0.405 | 1.50E−39 | A96 | Nr1i3 |
| Cox6a13 | 9.03E−44 | 0.274547323 | 0.943 | 0.862 | 2.24E−39 | A96 | Cox6a1 |
| Creg12 | 1.75E−43 | 0.251687315 | 0.985 | 0.919 | 4.34E−39 | A96 | Creg1 |
| Acaa1b2 | 3.05E−43 | 0.281984752 | 0.986 | 0.919 | 7.56E−39 | A96 | Acaa1b |
| Cyp7a1 | 1.08E−42 | 0.378826941 | 0.398 | 0.199 | 2.68E−38 | A96 | Cyp7a1 |
| Cyp2c542 | 1.49E−42 | 0.344687089 | 0.86 | 0.726 | 3.70E−38 | A96 | Cyp2c54 |
| Cyp2c502 | 4.24E−42 | 0.324188529 | 0.943 | 0.847 | 1.05E−37 | A96 | Cyp2c50 |
| Snord1182 | 9.05E−42 | 0.378239005 | 0.564 | 0.36 | 2.25E−37 | A96 | Snord118 |
| Asl2 | 9.71E−42 | 0.275035314 | 0.896 | 0.811 | 2.41E−37 | A96 | Asl |
| Csad2 | 9.95E−42 | 0.420651804 | 0.843 | 0.684 | 2.47E−37 | A96 | Csad |
| Sc4mol2 | 2.31E−41 | 0.336092423 | 0.781 | 0.631 | 5.74E−37 | A96 | Sc4mol |
| Igfbp13 | 2.40E−41 | 0.260251838 | 0.826 | 0.691 | 5.95E−37 | A96 | Igfbp1 |
| Gabarapl11 | 6.61E−41 | 0.293237647 | 0.881 | 0.78 | 1.64E−36 | A96 | Gabarapl1 |
| Tsc22d3 | 8.65E−41 | 0.336254391 | 0.534 | 0.338 | 2.15E−36 | A96 | Tsc22d3 |
| Acot11 | 1.16E−40 | 0.310587102 | 0.459 | 0.257 | 2.88E−36 | A96 | Acot1 |
| Gyk | 2.01E−40 | 0.313401573 | 0.616 | 0.441 | 5.00E−36 | A96 | Gyk |
| Decr22 | 3.24E−40 | 0.301753937 | 0.813 | 0.664 | 8.03E−36 | A96 | Decr2 |
| Cth1 | 3.77E−40 | 0.296643035 | 0.944 | 0.826 | 9.37E−36 | A96 | Cth |
| Pon12 | 4.32E−40 | 0.289869231 | 0.965 | 0.888 | 1.07E−35 | A96 | Pon1 |
| Cd36 | 9.83E−40 | 0.312878157 | 0.299 | 0.131 | 2.44E−35 | A96 | Cd36 |
| Aplp2 | 1.81E−39 | 0.290277561 | 0.826 | 0.67 | 4.48E−35 | A96 | Aplp2 |
| Agpat61 | 2.42E−38 | 0.314279412 | 0.743 | 0.584 | 6.01E−34 | A96 | Agpat6 |
| Agt2 | 2.70E−38 | 0.255008722 | 0.912 | 0.827 | 6.71E−34 | A96 | Agt |
| Hmgcl3 | 1.80E−37 | 0.274375388 | 0.871 | 0.739 | 4.47E−33 | A96 | Hmgcl |
| Gstt31 | 3.07E−37 | 0.302584013 | 0.541 | 0.347 | 7.61E−33 | A96 | Gstt3 |
| Aqp91 | 3.47E−37 | 0.317770961 | 0.801 | 0.664 | 8.62E−33 | A96 | Aqp9 |
| Akr1d12 | 6.11E−37 | 0.299364499 | 0.721 | 0.567 | 1.52E−32 | A96 | Akr1d1 |
| Slc4a41 | 7.42E−37 | 0.297654558 | 0.609 | 0.42 | 1.84E−32 | A96 | Slc4a4 |
| Ptms1 | 8.05E−37 | 0.286253315 | 0.806 | 0.71 | 2.00E−32 | A96 | Ptms |
| Rgn2 | 9.16E−37 | 0.368626566 | 0.899 | 0.786 | 2.27E−32 | A96 | Rgn |
| Lcat2 | 9.19E−37 | 0.261581087 | 0.886 | 0.786 | 2.28E−32 | A96 | Lcat |
| Hpgd1 | 1.45E−36 | 0.341178641 | 0.588 | 0.41 | 3.59E−32 | A96 | Hpgd |
| Hmgcs11 | 1.88E−36 | 0.325148716 | 0.858 | 0.718 | 4.67E−32 | A96 | Hmgcs1 |
| Rdh16 | 2.69E−36 | 0.347667254 | 0.494 | 0.31 | 6.69E−32 | A96 | Rdh16 |
| Cyp2c372 | 2.78E−36 | 0.414355767 | 0.71 | 0.521 | 6.89E−32 | A96 | Cyp2c37 |
| Tob11 | 1.03E−35 | 0.295824005 | 0.649 | 0.487 | 2.55E−31 | A96 | Tobi |
| Kif1b | 2.07E−35 | 0.296097725 | 0.686 | 0.507 | 5.15E−31 | A96 | Kif1b |
| Bnip33 | 3.84E−35 | 0.320840361 | 0.716 | 0.558 | 9.53E−31 | A96 | Bnip3 |
| Ndufa31 | 4.91E−35 | 0.274293228 | 0.737 | 0.598 | 1.22E−30 | A96 | Ndufa3 |
| Apoc22 | 5.09E−35 | 0.278106979 | 0.901 | 0.815 | 1.26E−30 | A96 | Apoc2 |
| Aldh9a11 | 7.77E−35 | 0.258476419 | 0.884 | 0.786 | 1.93E−30 | A96 | Aldh9a1 |
| Cpt1a1 | 4.44E−34 | 0.262562089 | 0.837 | 0.693 | 1.10E−29 | A96 | Cpt1a |
| Hsd17b7 | 7.40E−34 | 0.30278955 | 0.56 | 0.386 | 1.84E−29 | A96 | Hsd17b7 |
| Acat12 | 7.72E−34 | 0.255795606 | 0.917 | 0.852 | 1.92E−29 | A96 | Acat1 |
| D4Wsu53e | 8.98E−34 | 0.292811118 | 0.597 | 0.413 | 2.23E−29 | A96 | D4Wsu53e |
| Cox5a2 | 9.78E−34 | 0.272834577 | 0.813 | 0.715 | 2.43E−29 | A96 | Cox5a |
| Gsta21 | 1.28E−33 | 0.31098189 | 0.522 | 0.356 | 3.17E−29 | A96 | Gsta2 |
| Cxcl121 | 1.54E−33 | 0.28214551 | 0.635 | 0.48 | 3.82E−29 | A96 | Cxcl12 |
| Baat1 | 1.66E−33 | 0.272783991 | 0.862 | 0.769 | 4.13E−29 | A96 | Baat |
| Cyp2c681 | 1.69E−33 | 0.293077044 | 0.695 | 0.527 | 4.20E−29 | A96 | Cyp2c68 |
| Hsd17b103 | 4.80E−33 | 0.280202519 | 0.841 | 0.702 | 1.19E−28 | A96 | Hsd17b10 |
| Ndufa72 | 7.78E−33 | 0.260000131 | 0.818 | 0.695 | 1.93E−28 | A96 | Ndufa7 |
| Rdx2 | 1.06E−32 | 0.256615237 | 0.824 | 0.683 | 2.63E−28 | A96 | Rdx |
| Gas51 | 1.39E−32 | 0.282282316 | 0.68 | 0.525 | 3.46E−28 | A96 | Gas5 |
| G6pc1 | 2.86E−32 | 0.254149322 | 0.86 | 0.728 | 7.09E−28 | A96 | G6pc |
| Cpeb4 | 3.54E−32 | 0.301102867 | 0.527 | 0.357 | 8.79E−28 | A96 | Cpeb4 |
| Tgoln11 | 3.58E−32 | 0.280892109 | 0.756 | 0.626 | 8.89E−28 | A96 | Tgoln1 |
| Tmed51 | 4.26E−32 | 0.252317476 | 0.714 | 0.569 | 1.06E−27 | A96 | Tmed5 |
| Por3 | 4.35E−32 | 0.286727601 | 0.858 | 0.701 | 1.08E−27 | A96 | Por |
| Abcc31 | 9.20E−32 | 0.274329395 | 0.733 | 0.572 | 2.28E−27 | A96 | Abcc3 |
| Etfdh | 9.29E−32 | 0.263880086 | 0.811 | 0.706 | 2.31E−27 | A96 | Etfdh |
| Arl4a | 1.24E−31 | 0.270622637 | 0.517 | 0.342 | 3.09E−27 | A96 | Arl4a |
| Cpox | 1.57E−31 | 0.322921483 | 0.634 | 0.486 | 3.91E−27 | A96 | Cpox |
| Zfp36l1 | 2.69E−31 | 0.296352274 | 0.601 | 0.447 | 6.69E−27 | A96 | Zfp36l1 |
| Dhdh1 | 2.78E−31 | 0.26256299 | 0.734 | 0.615 | 6.90E−27 | A96 | Dhdh |
| Slco1a41 | 5.51E−31 | 0.304909008 | 0.507 | 0.336 | 1.37E−26 | A96 | Slco1a4 |
| Cyp2c442 | 1.06E−30 | 0.273539159 | 0.828 | 0.71 | 2.64E−26 | A96 | Cyp2c44 |
| Ctage51 | 1.29E−29 | 0.253519236 | 0.837 | 0.716 | 3.22E−25 | A96 | Ctage5 |
| Mia31 | 2.42E−29 | 0.260448824 | 0.81 | 0.659 | 6.02E−25 | A96 | Mia3 |
| Slc25a201 | 3.50E−29 | 0.25073463 | 0.671 | 0.543 | 8.69E−25 | A96 | Slc25a20 |
| G0s22 | 7.31E−29 | 0.265563376 | 0.525 | 0.35 | 1.81E−24 | A96 | G0s2 |
| Oat2 | 9.25E−29 | 0.490397584 | 0.653 | 0.478 | 2.30E−24 | A96 | Oat |
| F5 | 5.35E−28 | 0.256577637 | 0.858 | 0.754 | 1.33E−23 | A96 | F5 |
| Cox8a3 | 1.48E−27 | 0.251853134 | 0.737 | 0.626 | 3.67E−23 | A96 | Cox8a |
| Gm49521 | 2.35E−27 | 0.258080982 | 0.663 | 0.536 | 5.82E−23 | A96 | Gm4952 |
| Pxmp4 | 1.07E−26 | 0.255478862 | 0.687 | 0.556 | 2.65E−22 | A96 | Pxmp4 |
| Ppp1r3b1 | 1.27E−26 | 0.250974794 | 0.536 | 0.377 | 3.16E−22 | A96 | Ppp1r3b |
| Fmo1 | 3.23E−26 | 0.264953339 | 0.606 | 0.446 | 8.01E−22 | A96 | Fmo1 |
| Zfp911 | 9.57E−26 | 0.277539527 | 0.693 | 0.56 | 2.38E−21 | A96 | Zfp91 |
| Apol7a1 | 3.42E−23 | 0.25175601 | 0.559 | 0.42 | 8.50E−19 | A96 | Apo17a |
| Ces1e | 5.02E−22 | 0.256602575 | 0.71 | 0.608 | 1.25E−17 | A96 | Ces1e |
| Gadd45b | 5.16E−21 | 0.259769736 | 0.452 | 0.311 | 1.28E−16 | A96 | Gadd45b |
| Etnppl3 | 6.58E−13 | 0.315432141 | 0.677 | 0.619 | 1.63E−08 | A96 | Etnppl |
| Mup174 | 0 | 2.016622219 | 1 | 0.947 | 0 | UT | Mup17 |
| Mup94 | 0 | 1.914490248 | 0.976 | 0.594 | 0 | UT | Mup9 |
| Mup64 | 0 | 1.881843585 | 0.992 | 0.804 | 0 | UT | Mup6 |
| Mup194 | 0 | 1.785867292 | 1 | 0.916 | 0 | UT | Mup19 |
| Mup115 | 0 | 1.728428444 | 1 | 0.84 | 0 | UT | Mup11 |
| Mup184 | 0 | 1.638566323 | 0.997 | 0.838 | 0 | UT | Mup18 |
| Mup54 | 0 | 1.341986627 | 0.839 | 0.541 | 0 | UT | Mup5 |
| Mup154 | 0 | 1.259561807 | 0.617 | 0.166 | 0 | UT | Mup15 |
| Mup164 | 0 | 1.12499966 | 0.934 | 0.594 | 0 | UT | Mup16 |
| mt-Nd44 | 0 | 1.115678214 | 0.998 | 0.942 | 0 | UT | mt-Nd4 |
| mt-Co24 | 0 | 1.077728564 | 0.629 | 0.157 | 0 | UT | mt-Co2 |
| Gm137754 | 0 | 1.061267629 | 0.761 | 0.306 | 0 | UT | Gm13775 |
| mt-Co14 | 0 | 0.921896864 | 0.999 | 0.977 | 0 | UT | mt-Co1 |
| mt-Cytb3 | 0 | 0.877546618 | 0.999 | 0.971 | 0 | UT | mt-Cytb |
| Mup214 | 0 | 0.872348794 | 0.929 | 0.702 | 0 | UT | Mup21 |
| mt-Nd23 | 0 | 0.832515437 | 0.999 | 0.978 | 0 | UT | mt-Nd2 |
| Selenbp23 | 0 | 0.830430695 | 0.937 | 0.751 | 0 | UT | Selenbp2 |
| mt-Nd54 | 0 | 0.779679711 | 0.995 | 0.942 | 0 | UT | mt-Nd5 |
| mt-Nd13 | 0 | 0.759986171 | 1 | 0.978 | 0 | UT | mt-Nd1 |
| mt-Rnr22 | 0 | 0.545826779 | 1 | 1 | 0 | UT | mt-Rnr2 |
| Mup32 | 0 | 0.542484173 | 1 | 0.999 | 0 | UT | Mup3 |
| mt-Co34 | 1.54E−290 | 0.958403256 | 0.603 | 0.198 | 3.82E−286 | UT | mt-Co3 |
| Serpina1e1 | 1.35E−287 | 0.468116476 | 0.999 | 0.99 | 3.36E−283 | UT | Serpina1e |
| Akr1c62 | 4.68E−230 | 0.504664681 | 0.971 | 0.92 | 1.16E−225 | UT | Akr1c6 |
| Ankrd554 | 6.63E−225 | 1.230858809 | 0.696 | 0.451 | 1.65E−220 | UT | Ankrd55 |
| Mup43 | 8.62E−211 | 1.000765415 | 0.898 | 0.716 | 2.14E−206 | UT | Mup4 |
| mt-Rnr13 | 1.37E−205 | 0.353829755 | 1 | 1 | 3.40E−201 | UT | mt-Rnr1 |
| Fabp11 | 3.44E−187 | 0.30775699 | 1 | 0.992 | 8.54E−183 | UT | Fabp1 |
| Elovl33 | 5.11E−177 | 0.522135473 | 0.827 | 0.65 | 1.27E−172 | UT | Elovl3 |
| Gsta32 | 4.62E−168 | 0.404911812 | 0.956 | 0.958 | 1.15E−163 | UT | Gsta3 |
| Mup74 | 2.96E−167 | 0.601531682 | 0.998 | 0.937 | 7.35E−163 | UT | Mup7 |
| Nudt74 | 3.51E−163 | 0.6192372 | 0.83 | 0.691 | 8.71E−159 | UT | Nudt7 |
| Serpina1a1 | 1.94E−157 | 0.33473551 | 0.991 | 0.96 | 4.81E−153 | UT | Serpina1a |
| Uqcrq2 | 7.40E−157 | 0.410137125 | 0.951 | 0.902 | 1.84E−152 | UT | Uqcrq |
| Car32 | 2.61E−147 | 0.376245944 | 0.974 | 0.956 | 6.47E−143 | UT | Car3 |
| Ces3a2 | 1.36E−137 | 0.317352764 | 0.967 | 0.93 | 3.38E−133 | UT | Ces3a |
| Mup84 | 1.52E−136 | 0.726763962 | 0.373 | 0.13 | 3.77E−132 | UT | Mup8 |
| Adh11 | 1.12E−132 | 0.31221001 | 0.96 | 0.941 | 2.79E−128 | UT | Adh1 |
| Prdx12 | 2.41E−131 | 0.349862194 | 0.927 | 0.878 | 5.99E−127 | UT | Prdx1 |
| Gstp12 | 4.61E−130 | 0.608807368 | 0.822 | 0.702 | 1.14E−125 | UT | Gstp1 |
| Hsd3b72 | 3.94E−128 | 0.450130172 | 0.798 | 0.693 | 9.79E−124 | UT | Hsd3b7 |
| Acaa1b3 | 1.79E−119 | 0.315795941 | 0.969 | 0.883 | 4.44E−115 | UT | Acaa1b |
| Mup201 | 2.74E−117 | 0.372499315 | 1 | 0.992 | 6.81E−113 | UT | Mup20 |
| Mup133 | 3.18E−117 | 0.624965563 | 0.546 | 0.351 | 7.90E−113 | UT | Mup13 |
| Rps142 | 1.62E−111 | 0.336423845 | 0.917 | 0.856 | 4.03E−107 | UT | Rsp14 |
| Cyb52 | 5.75E−109 | 0.284450423 | 0.983 | 0.972 | 1.43E−104 | UT | Cyb5 |
| 2810007J24Rik1 | 3.82E−107 | 0.287090402 | 0.943 | 0.909 | 9.49E−103 | UT | 2810007J24Rik |
| Hsd3b54 | 2.16E−104 | 0.471194244 | 0.587 | 0.367 | 5.37E−100 | UT | Hsd3b5 |
| Mup124 | 3.61E−104 | 0.371327569 | 0.86 | 0.665 | 8.97E−100 | UT | Mup12 |
| Cox4i12 | 2.61E−94 | 0.262749367 | 0.958 | 0.941 | 6.48E−90 | UT | Cox4i1 |
| Oaz11 | 5.51E−93 | 0.394430119 | 0.75 | 0.651 | 1.37E−88 | UT | Oaz1 |
| Tmem2052 | 1.10E−91 | 0.295263774 | 0.934 | 0.877 | 2.74E−87 | UT | Tmem205 |
| Serpina3c3 | 4.51E−84 | 0.425256202 | 0.385 | 0.208 | 1.12E−79 | UT | Serpina3c |
| Ugt2b11 | 1.86E−83 | 0.30063164 | 0.871 | 0.817 | 4.62E−79 | UT | Ugt2b1 |
| Rps23 | 3.51E−80 | 0.379370911 | 0.757 | 0.637 | 8.73E−76 | UT | Rps2 |
| Inmt2 | 1.03E−77 | 0.366227618 | 0.884 | 0.871 | 2.55E−73 | UT | Inmt |
| Cox6c2 | 1.98E−73 | 0.343178548 | 0.884 | 0.808 | 4.92E−69 | UT | Cox6c |
| Ndufb112 | 7.98E−73 | 0.352187855 | 0.735 | 0.637 | 1.98E−68 | UT | Ndufb11 |
| Cyp7b13 | 1.18E−72 | 0.351246722 | 0.757 | 0.654 | 2.92E−68 | UT | Cyp7b1 |
| Cyp1a22 | 2.21E−72 | 0.303458132 | 0.794 | 0.621 | 5.49E−68 | UT | Cyp1a2 |
| C8g2 | 9.19E−72 | 0.258246671 | 0.91 | 0.859 | 2.28E−67 | UT | C8g |
| Lifr3 | 5.26E−70 | 0.353193545 | 0.694 | 0.608 | 1.31E−65 | UT | Lifr |
| Hamp3 | 1.12E−69 | 0.274177459 | 0.884 | 0.718 | 2.79E−65 | UT | Hamp |
| Cfhr21 | 1.47E−66 | 0.329112182 | 0.868 | 0.854 | 3.64E−62 | UT | Cfhr2 |
| Mup143 | 2.61E−61 | 0.524421073 | 0.616 | 0.469 | 6.49E−57 | UT | Mup14 |
| Aadac2 | 2.88E−59 | 0.324280061 | 0.86 | 0.849 | 7.15E−55 | UT | Aadac |
| Keg13 | 4.07E−58 | 0.385687903 | 0.545 | 0.441 | 1.01E−53 | UT | Keg1 |
| Cyp2a52 | 7.24E−58 | 0.327337224 | 0.555 | 0.388 | 1.80E−53 | UT | Cyp2a5 |
| Chchd22 | 3.96E−57 | 0.257495549 | 0.916 | 0.874 | 9.83E−53 | UT | Chchd2 |
| Ces3b1 | 1.20E−56 | 0.283988668 | 0.787 | 0.727 | 2.98E−52 | UT | Ces3b |
| Ttc39c2 | 4.98E−56 | 0.318694789 | 0.642 | 0.573 | 1.24E−51 | UT | Ttc39c |
| Gstm32 | 2.30E−53 | 0.47361305 | 0.414 | 0.269 | 5.70E−49 | UT | Gstm3 |
| Gadd45g1 | 9.92E−53 | 0.340515267 | 0.639 | 0.503 | 2.46E−48 | UT | Gadd45g |
| Ndufa42 | 7.60E−52 | 0.261719376 | 0.889 | 0.851 | 1.89E−47 | UT | Ndufa4 |
| Cml11 | 7.90E−52 | 0.295319392 | 0.757 | 0.709 | 1.96E−47 | UT | Cml1 |
| Thrsp3 | 3.45E−50 | 0.460250446 | 0.627 | 0.583 | 8.56E−46 | UT | Thrsp |
| mt-Atp62 | 2.06E−48 | 0.419380785 | 0.452 | 0.311 | 5.11E−44 | UT | mt-Atp6 |
| Rpl321 | 1.00E−47 | 0.30384302 | 0.757 | 0.688 | 2.49E−43 | UT | Rpl32 |
| Hspe11 | 2.75E−44 | 0.308522832 | 0.557 | 0.462 | 6.83E−40 | UT | Hspe1 |
| Hsd3b31 | 2.62E−41 | 0.282851137 | 0.605 | 0.519 | 6.51E−37 | UT | Hsd3b3 |
| Serpina122 | 6.83E−41 | 0.279022117 | 0.492 | 0.362 | 1.70E−36 | UT | Serpina12 |
| Prdx41 | 1.19E−38 | 0.290503348 | 0.607 | 0.55 | 2.95E−34 | UT | Prdx4 |
| Mup24 | 3.60E−37 | 0.446904247 | 0.392 | 0.285 | 8.95E−33 | UT | Mup2 |
| Cox7a22 | 2.72E−36 | 0.251096024 | 0.784 | 0.722 | 6.76E−32 | UT | Cox7a2 |
| Cyp2b102 | 2.41E−35 | 0.502782767 | 0.542 | 0.493 | 5.98E−31 | UT | Cyp2b10 |
| Ndufs62 | 3.36E−32 | 0.272052906 | 0.724 | 0.678 | 8.33E−28 | UT | Ndufs6 |
| Ndufb41 | 1.61E−29 | 0.253090085 | 0.49 | 0.409 | 3.99E−25 | UT | Ndufb4 |
| Rpl36al1 | 8.10E−27 | 0.2702077 | 0.551 | 0.495 | 2.01E−22 | UT | Rpl36al |
| Mup116 | 1.57E−24 | 0.470008649 | 0.583 | 0.522 | 3.91E−20 | UT | Mup1 |
| Rps152 | 4.98E−19 | 0.272193403 | 0.528 | 0.49 | 1.24E−14 | UT | Rps15 |
| Ubb | 5.31E−19 | 0.264147083 | 0.45 | 0.394 | 1.32E−14 | UT | Ubb |
| Ftl1 | 2.27E−15 | 0.309991928 | 0.409 | 0.367 | 5.64E−11 | UT | Ftl1 |

Surprisingly, Applicants noticed many of the response genes have very similar patterns of upregulation in both injury models, including upregulation of genes related to damage response, redox, liver function (Alb) and pathway enrichments shared between APAP and PH as shown in Table 11. Upregulation of anti-oxidative stress genes unsurprisingly follows APAP exposure, in which involvement of ROS is a major cause of injury. However, upregulation of select anti-oxidative stress genes within the PH model, which does not involve a component of oxidative stress, suggests these and other shared genes may participate in a general insult-response module, which is activated in response to many types of damage. Pathway enrichments unique to APAP are provided in Table 9, Pathway enrichments unique to PH, in Table 10.

TABLE 9
Pathway enrichments unique to APAP.

| | Genes. | Stat.non. | p.non. | padj.non. | Genes. | Stat.mix. | p.mix. | padj.mix. | Genes. | Stat.mix. | p.mix. | padj.mix. |
|---|---|---|---|---|---|---|---|---|---|---|---|---|
| | tot._{—} | dir.._{—} | dir.._{—} | dir.._{—} | up._{—} | dir.up._{—} | dir.up._{—} | dir.up._{—} | down._{—} | dir.dn._{—} | dir.dn._{—} | dir.dn._{—} |
| | APAP | APAP | APAP | APAP | APAP | APAP | APAP | APAP | APAP | APAP | APAP | APAP |
| KEGG_PPAR_SIGNALING_PATHWAY | 22 | 7823.4 | 0.00 | 0.02 | 9 | 3462.1 | 0.00 | 0.04 | 13 | 4361.3 | 0.00 | 0.57 |
| KEGG_COMPLEMENT_AND_COAGULATION_CASCADES | 26 | 8046.9 | 0.00 | 0.06 | 20 | 6277.6 | 0.01 | 0.10 | 6 | 1769.3 | 0.11 | 1.00 |
| REACTOME_PPARA_ACTIVATES_GENE_EXPRESSION | 23 | 7289.1 | 0.00 | 0.06 | 17 | 5546.6 | 0.01 | 0.09 | 6 | 1742.5 | 0.12 | 1.00 |
| REACTOME_BIOLOGICAL_OXIDATIONS | 30 | 9098.2 | 0.00 | 0.06 | 12 | 3471.3 | 0.10 | 0.45 | 18 | 5627 | 0.00 | 0.57 |
| REACTOME_METABOLISM_OF_LIPIDS_AND_LIPOPROTEINS | 101 | 28033 | 0.00 | 0.04 | 67 | 20622 | 0.00 | 0.01 | 34 | 7410.2 | 0.39 | 1.00 |
| SENGUPTA_NASOPHARYNGEAL_CARCINOMA_DN | 23 | 7693.5 | 0.00 | 0.04 | 15 | 5172.6 | 0.00 | 0.05 | 8 | 2520.9 | 0.03 | 0.94 |
| TIEN_INTESTINE_PROBIOTICS_24 HR_DN | 62 | 17374 | 0.00 | 0.06 | 47 | 14200 | 0.00 | 0.03 | 15 | 3174.2 | 0.48 | 1.00 |
| TARTE_PLASMA_CELL_VS_PLASMABLAST_UP | 46 | 13841 | 0.00 | 0.04 | 25 | 7761 | 0.01 | 0.08 | 21 | 6079.9 | 0.01 | 0.69 |
| LEE_LIVER_CANCER_DENA_DN | 36 | 10808 | 0.00 | 0.04 | 7 | 2520 | 0.02 | 0.16 | 29 | 8288.4 | 0.00 | 0.69 |
| HSIAO_LIVER_SPECIFIC_GENES | 131 | 42605 | 0.00 | 0.02 | 73 | 26630 | 0.00 | 0.01 | 58 | 15975 | 0.00 | 0.39 |
| MUNSHI_MULTIPLE_MYELOMA_UP | 20 | 6523.9 | 0.00 | 0.06 | 12 | 4427.7 | 0.00 | 0.03 | 8 | 2096.2 | 0.18 | 1.00 |
| TAKAO_RESPONSE_TO_UVB_RADIATION_UP | 26 | 7896.4 | 0.00 | 0.09 | 8 | 2743.9 | 0.02 | 0.18 | 18 | 5152.5 | 0.02 | 0.82 |
| KAAB_HEART_ATRIUM_VS_VENTRICLE_UP | 22 | 6839.7 | 0.00 | 0.09 | 15 | 5295.8 | 0.00 | 0.04 | 7 | 1543.9 | 0.43 | 1.00 |
| RODWELL_AGING_KIDNEY_NO_BLOOD_UP | 25 | 7724.8 | 0.00 | 0.06 | 19 | 6355.2 | 0.00 | 0.04 | 6 | 1369.6 | 0.38 | 1.00 |
| CREIGHTON_ENDOCRINE_THERAPY_RESISTANCE_3 | 91 | 23927 | 0.00 | 0.10 | 74 | 19930 | 0.01 | 0.12 | 17 | 3997.3 | 0.25 | 1.00 |
| ACEVEDO_LIVER_CANCER_DN | 138 | 39248 | 0.00 | 0.02 | 80 | 26286 | 0.00 | 0.01 | 58 | 12962 | 0.25 | 1.00 |
| SMID_BREAST_CANCER_BASAL_DN | 82 | 22484 | 0.00 | 0.04 | 50 | 14455 | 0.00 | 0.06 | 32 | 8028.5 | 0.07 | 1.00 |
| BOCHKIS_FOXA2_TARGETS | 106 | 27826 | 0.00 | 0.08 | 63 | 16811 | 0.03 | 0.22 | 43 | 11015 | 0.02 | 0.85 |
| BOQUEST_STEM_CELL_UP | 26 | 8561.2 | 0.00 | 0.04 | 16 | 5723 | 0.00 | 0.03 | 10 | 2838.2 | 0.08 | 1.00 |
| SWEET_LUNG_CANCER_KRAS_DN | 55 | 15677 | 0.00 | 0.06 | 36 | 10466 | 0.01 | 0.10 | 19 | 5211.5 | 0.04 | 0.94 |
| ZHANG_TLX_TARGETS_60 HR_UP | 32 | 9533.3 | 0.00 | 0.09 | 23 | 7214.3 | 0.01 | 0.08 | 9 | 2318.9 | 0.18 | 1.00 |
| BOYAULT_LIVER_CANCER_SUBCLASS_G123_DN | 23 | 7152.8 | 0.00 | 0.08 | 13 | 4470.2 | 0.00 | 0.07 | 10 | 2682.6 | 0.13 | 1.00 |
| CHIANG_LIVER_CANCER_SUBCLASS_PROLIFERATION_DN | 77 | 20983 | 0.00 | 0.06 | 33 | 11045 | 0.00 | 0.01 | 44 | 9938 | 0.25 | 1.00 |
| YAMASHITA_LIVER_CANCER_STEM_CELL_DN | 33 | 10027 | 0.00 | 0.06 | 17 | 5107.1 | 0.03 | 0.27 | 16 | 4919.9 | 0.01 | 0.69 |
| WOO_LIVER_CANCER_RECURRENCE_DN | 38 | 11695 | 0.00 | 0.03 | 17 | 5508.3 | 0.01 | 0.10 | 21 | 6186.7 | 0.01 | 0.69 |
| HOSHIDA_LIVER_CANCER_SUBCLASS_S3 | 111 | 30365 | 0.00 | 0.04 | 54 | 17777 | 0.00 | 0.01 | 57 | 12588 | 0.29 | 1.00 |
| CAIRO_HEPATOBLASTOMA_DN | 102 | 27531 | 0.00 | 0.04 | 53 | 16333 | 0.00 | 0.03 | 49 | 11198 | 0.20 | 1.00 |
| CAIRO_HEPATOBLASTOMA_CLASSES_DN | 45 | 12828 | 0.00 | 0.09 | 26 | 7144.5 | 0.07 | 0.38 | 19 | 5683.9 | 0.01 | 0.69 |
| CAIRO_LIVER_DEVELOPMENT_DN | 100 | 28189 | 0.00 | 0.03 | 63 | 20717 | 0.00 | 0.01 | 37 | 7471.8 | 0.64 | 1.00 |
| SERVITJA_LIVER_HNF1A_TARGETS_UP | 20 | 6447.4 | 0.00 | 0.06 | 15 | 4903.8 | 0.01 | 0.11 | 5 | 1543.6 | 0.09 | 1.00 |
| NABA_MATRISOME | 69 | 20317 | 0.00 | 0.02 | 59 | 18644 | 0.00 | 0.01 | 10 | 1673.9 | 0.81 | 1.00 |
| VILLANUEVA_LIVER_CANCER_KRT19_DN | 24 | 7562.1 | 0.00 | 0.06 | 11 | 4125.1 | 0.00 | 0.04 | 13 | 3437 | 0.11 | 1.00 |
| GO_FATTY_ACID_CATABOLIC_PROCESS | 28 | 8095 | 0.01 | 0.08 | 15 | 5348.5 | 0.00 | 0.01 | 13 | 2746.5 | 0.48 | 0.98 |
| GO_AMMONIUM_ION_METABOLIC_PROCESS | 34 | 11097 | 0.00 | 0.01 | 21 | 7993.5 | 0.00 | 0.00 | 13 | 3103.2 | 0.26 | 0.83 |
| GO_PROTEIN_ACTIVATION_CASCADE | 26 | 8061.5 | 0.00 | 0.03 | 20 | 6309.4 | 0.01 | 0.04 | 6 | 1752.1 | 0.10 | 0.64 |
| GO_REGULATION_OF_WOUND_HEALING | 21 | 6945.8 | 0.00 | 0.01 | 18 | 6360.3 | 0.00 | 0.01 | 3 | 585.59 | 0.56 | 1.00 |
| GO_RESPONSE_TO_ESTRADIOL | 24 | 7016.7 | 0.02 | 0.09 | 17 | 5334.2 | 0.01 | 0.07 | 7 | 1682.5 | 0.30 | 0.86 |
| GO_RESPONSE_TO_ACID_CHEMICAL | 57 | 15642 | 0.00 | 0.04 | 37 | 10651 | 0.01 | 0.06 | 20 | 4991.6 | 0.13 | 0.66 |
| GO_STEROL_HOMEOSTASIS | 21 | 6247.9 | 0.02 | 0.09 | 11 | 3538.4 | 0.03 | 0.12 | 10 | 2709.5 | 0.11 | 0.64 |
| GO_LIPOPROTEIN_METABOLIC_PROCESS | 20 | 6956.1 | 0.00 | 0.01 | 14 | 4569.3 | 0.01 | 0.06 | 6 | 2386.8 | 0.00 | 0.26 |
| GO_FATTY_ACID_BETA_OXIDATION | 22 | 6503.2 | 0.01 | 0.09 | 12 | 4501.3 | 0.00 | 0.01 | 10 | 2001.9 | 0.57 | 1.00 |
| GO_PROTEIN_MATURATION | 32 | 9466.7 | 0.00 | 0.03 | 23 | 7517.9 | 0.00 | 0.02 | 9 | 1948.8 | 0.44 | 0.96 |
| GO_POSITIVE_REGULATION_OF_LOCOMOTION | 38 | 10715 | 0.01 | 0.07 | 33 | 9976.1 | 0.00 | 0.03 | 5 | 739.16 | 0.81 | 1.00 |
| GO_ORGANIC_HYDROXY_COMPOUND_METABOLIC_PROCESS | 113 | 30659 | 0.00 | 0.01 | 61 | 19192 | 0.00 | 0.00 | 52 | 11468 | 0.31 | 0.88 |
| GO_ORGANIC_ACID_BIOSYNTHETIC_PROCESS | 69 | 18105 | 0.01 | 0.09 | 30 | 9094.8 | 0.00 | 0.04 | 39 | 9010 | 0.19 | 0.74 |
| GO_MONOCARBOXYLIC_ACID_METABOLIC_PROCESS | 115 | 30536 | 0.00 | 0.01 | 55 | 17539 | 0.00 | 0.00 | 60 | 12996 | 0.37 | 0.91 |
| GO_RESPONSE_TO_INORGANIC_SUBSTANCE | 88 | 22912 | 0.01 | 0.06 | 59 | 15860 | 0.02 | 0.11 | 29 | 7052.4 | 0.12 | 0.66 |
| GO_REGULATION_OF_VESICLE_MEDIATED_TRANSPORT | 34 | 11187 | 0.00 | 0.01 | 28 | 9240.7 | 0.00 | 0.01 | 6 | 1946.2 | 0.04 | 0.55 |
| GO_REGULATION_OF_FATTY_ACID_METABOLIC_PROCESS | 21 | 7538.8 | 0.00 | 0.00 | 14 | 5374.9 | 0.00 | 0.00 | 7 | 2163.9 | 0.06 | 0.56 |
| GO_ORGANIC_HYDROXY_COMPOUND_TRANSPORT | 31 | 9865.3 | 0.00 | 0.01 | 18 | 6306 | 0.00 | 0.01 | 13 | 3559.2 | 0.07 | 0.56 |
| GO_DEFENSE_RESPONSE | 121 | 32545 | 0.00 | 0.01 | 89 | 23374 | 0.02 | 0.09 | 32 | 9171.1 | 0.00 | 0.26 |
| GO_ORGANOPHOSPHATE_ESTER_TRANSPORT | 20 | 6642 | 0.00 | 0.01 | 13 | 3866.1 | 0.06 | 0.22 | 7 | 2775.9 | 0.00 | 0.26 |
| GO_LIPID_HOMEOSTASIS | 35 | 10260 | 0.00 | 0.03 | 21 | 6598.3 | 0.01 | 0.05 | 14 | 3661.5 | 0.11 | 0.64 |
| GO_SECRETION | 58 | 17186 | 0.00 | 0.00 | 48 | 15502 | 0.00 | 0.00 | 10 | 1684 | 0.80 | 1.00 |
| GO_ANION_TRANSPORT | 59 | 15946 | 0.01 | 0.06 | 38 | 11431 | 0.00 | 0.03 | 21 | 4515.4 | 0.44 | 0.95 |
| GO_REGULATION_OF_LIPID_BIOSYNTHETIC_PROCESS | 34 | 10760 | 0.00 | 0.01 | 23 | 7092.6 | 0.01 | 0.05 | 11 | 3667.3 | 0.01 | 0.35 |
| GO_RESPONSE_TO_XENOBIOTIC_STIMULUS | 21 | 6511 | 0.01 | 0.05 | 6 | 2131.7 | 0.02 | 0.12 | 15 | 4379.4 | 0.02 | 0.44 |
| GO_POSITIVE_REGULATION_OF_RESPONSE_TO_EXTERNAL_{—} | 30 | 9595.6 | 0.00 | 0.01 | 26 | 8757.6 | 0.00 | 0.01 | 4 | 837.98 | 0.48 | 0.98 |
| STIMULUS | | | | | | | | | | | | |
| GO_SMALL_MOLECULE_BIOSYNTHETIC_PROCESS | 106 | 28989 | 0.00 | 0.01 | 52 | 17188 | 0.00 | 0.00 | 54 | 11801 | 0.33 | 0.90 |
| GO_REGULATION_OF_RESPONSE_TO_EXTERNAL_STIMULUS | 89 | 25237 | 0.00 | 0.00 | 69 | 19702 | 0.00 | 0.01 | 20 | 5534.6 | 0.03 | 0.52 |
| GO_REGULATION_OF_LIPID_METABOLIC_PROCESS | 56 | 17326 | 0.00 | 0.00 | 34 | 10855 | 0.00 | 0.01 | 22 | 6471.7 | 0.01 | 0.35 |
| GO_AMINE_METABOLIC_PROCESS | 32 | 10016 | 0.00 | 0.01 | 17 | 6153.3 | 0.00 | 0.00 | 15 | 3862.4 | 0.13 | 0.66 |
| GO_AUTOPHAGY | 35 | 9748.3 | 0.02 | 0.10 | 26 | 7361.8 | 0.04 | 0.15 | 9 | 2386.5 | 0.15 | 0.70 |
| GO_REGULATION_OF_SECRETION | 56 | 15716 | 0.00 | 0.03 | 47 | 13424 | 0.01 | 0.04 | 9 | 2292.3 | 0.20 | 0.75 |
| GO_APOPTOTIC_SIGNALING_PATHWAY | 34 | 9850.6 | 0.01 | 0.05 | 26 | 7310 | 0.04 | 0.17 | 8 | 2540.6 | 0.03 | 0.50 |
| GO_ACUTE_INFLAMMATORY_RESPONSE | 25 | 8681.7 | 0.00 | 0.01 | 18 | 6505.8 | 0.00 | 0.00 | 7 | 2175.9 | 0.05 | 0.56 |
| GO_POSITIVE_REGULATION_OF_CATABOLIC_PROCESS | 61 | 17142 | 0.00 | 0.02 | 42 | 12406 | 0.00 | 0.03 | 19 | 4736.1 | 0.14 | 0.68 |
| GO_MAINTENANCE_OF_LOCATION | 22 | 7094.1 | 0.00 | 0.02 | 18 | 6373.1 | 0.00 | 0.01 | 4 | 721.06 | 0.62 | 1.00 |
| GO_POSITIVE_REGULATION_OF_PROTEIN_SECRETION | 21 | 6254.7 | 0.01 | 0.09 | 18 | 5654.2 | 0.01 | 0.06 | 3 | 600.47 | 0.53 | 1.00 |
| GO_ORGANIC_ACID_METABOLIC_PROCESS | 202 | 51076 | 0.00 | 0.02 | 100 | 29277 | 0.00 | 0.00 | 102 | 21799 | 0.40 | 0.94 |
| GO_RESPONSE_TO_OXYGEN_LEVELS | 35 | 9850.8 | 0.01 | 0.08 | 25 | 7215.4 | 0.03 | 0.12 | 10 | 2635.4 | 0.14 | 0.68 |
| GO_REGULATION_OF_CELLULAR_KETONE_METABOLIC_PROCESS | 37 | 10386 | 0.01 | 0.07 | 18 | 6076.1 | 0.00 | 0.02 | 19 | 4309.7 | 0.32 | 0.89 |
| GO_ORGANIC_ACID_CATABOLIC_PROCESS | 74 | 19313 | 0.01 | 0.09 | 32 | 10483 | 0.00 | 0.01 | 42 | 8830.5 | 0.49 | 0.98 |
| GO_REGULATION_OF_RESPONSE_TO_WOUNDING | 57 | 17902 | 0.00 | 0.00 | 45 | 14043 | 0.00 | 0.00 | 12 | 3858.7 | 0.01 | 0.38 |
| GO_REGULATION_OF_EXTRINSIC_APOPTOTIC_SIGNALING_{—} | 23 | 7361 | 0.00 | 0.01 | 20 | 6215.2 | 0.01 | 0.06 | 3 | 1145.7 | 0.04 | 0.53 |
| PATHWAY | | | | | | | | | | | | |
| GO_CELLULAR_LIPID_CATABOLIC_PROCESS | 42 | 13000 | 0.00 | 0.01 | 23 | 8176.2 | 0.00 | 0.00 | 19 | 4824.1 | 0.11 | 0.65 |
| GO_REGULATION_OF_REACTIVE_OXYGEN_SPECIES_METABOLIC_{—} | 23 | 6763.4 | 0.01 | 0.09 | 13 | 4359.2 | 0.01 | 0.05 | 10 | 2404.1 | 0.27 | 0.83 |
| PROCESS | | | | | | | | | | | | |
| GO_DETOXIFICATION | 23 | 7116.3 | 0.00 | 0.03 | 9 | 2877.1 | 0.04 | 0.17 | 14 | 4239.1 | 0.02 | 0.40 |
| GO_REGULATION_OF_PEPTIDASE_ACTIVITY | 53 | 14643 | 0.01 | 0.05 | 36 | 11427 | 0.00 | 0.01 | 17 | 3216.6 | 0.71 | 1.00 |
| GO_ORGANIC_ANION_TRANSPORT | 54 | 15332 | 0.00 | 0.02 | 35 | 11024 | 0.00 | 0.01 | 19 | 4308.2 | 0.32 | 0.89 |
| GO_INFLAMMATORY_RESPONSE | 58 | 17317 | 0.00 | 0.00 | 43 | 13110 | 0.00 | 0.01 | 15 | 4206.7 | 0.05 | 0.55 |
| GO_NEGATIVE_REGULATION_OF_CELL_DEATH | 107 | 28526 | 0.00 | 0.01 | 85 | 22340 | 0.02 | 0.11 | 22 | 6185.4 | 0.02 | 0.40 |
| GO_FATTY_ACID_METABOLIC_PROCESS | 69 | 19656 | 0.00 | 0.01 | 40 | 13112 | 0.00 | 0.00 | 29 | 6544.5 | 0.28 | 0.84 |
| GO_LIPID_METABOLIC_PROCESS | 196 | 52216 | 0.00 | 0.01 | 113 | 33135 | 0.00 | 0.00 | 83 | 19081 | 0.11 | 0.64 |
| GO_DIVALENT_INORGANIC_CATION_HOMEOSTASIS | 25 | 7615.9 | 0.00 | 0.04 | 19 | 5393.1 | 0.06 | 0.22 | 6 | 2222.8 | 0.01 | 0.35 |
| GO_REGULATION_OF_ENDOCYTOSIS | 20 | 6706.3 | 0.00 | 0.01 | 15 | 4807.9 | 0.01 | 0.07 | 5 | 1898.5 | 0.01 | 0.38 |
| GO_ALCOHOL_METABOLIC_PROCESS | 81 | 21911 | 0.00 | 0.03 | 44 | 13202 | 0.00 | 0.01 | 37 | 8709 | 0.15 | 0.71 |
| GO_IMMUNE_RESPONSE | 86 | 22263 | 0.01 | 0.09 | 63 | 15638 | 0.16 | 0.41 | 23 | 6624.3 | 0.01 | 0.38 |
| GO_CELLULAR_LIPID_METABOLIC_PROCESS | 148 | 40329 | 0.00 | 0.00 | 88 | 26491 | 0.00 | 0.00 | 60 | 13838 | 0.14 | 0.68 |
| GO_MONOCARBOXYLIC_ACID_TRANSPORT | 28 | 8200.8 | 0.01 | 0.06 | 18 | 6208.2 | 0.00 | 0.01 | 10 | 1992.7 | 0.58 | 1.00 |
| GO_NEGATIVE_REGULATION_OF_APOPTOTIC_SIGNALING_{—} | 30 | 9140.9 | 0.00 | 0.02 | 24 | 7115.5 | 0.02 | 0.09 | 6 | 2025.3 | 0.03 | 0.50 |
| PATHWAY | | | | | | | | | | | | |
| GO_MULTICELLULAR_ORGANISMAL_HOMEOSTASIS | 26 | 8484.4 | 0.00 | 0.01 | 20 | 6159.3 | 0.01 | 0.07 | 6 | 2325.1 | 0.00 | 0.26 |
| GO_ORGANIC_HYDROXY_COMPOUND_BIOSYNTHETIC_PROCESS | 48 | 13228 | 0.01 | 0.06 | 24 | 8180.2 | 0.00 | 0.01 | 24 | 5047.9 | 0.50 | 0.98 |
| GO_REGULATION_OF_CELL_DEATH | 159 | 40571 | 0.00 | 0.03 | 126 | 32290 | 0.02 | 0.09 | 33 | 8281.4 | 0.07 | 0.56 |
| GO_LIPID_CATABOLIC_PROCESS | 54 | 15902 | 0.00 | 0.01 | 31 | 10400 | 0.00 | 0.00 | 23 | 5502.7 | 0.19 | 0.74 |
| GO_POSITIVE_REGULATION_OF_LIPID_METABOLIC_PROCESS | 30 | 11217 | 0.00 | 0.00 | 21 | 7735.3 | 0.00 | 0.01 | 9 | 3481.6 | 0.00 | 0.26 |
| GO_REGULATION_OF_STEROID_METABOLIC_PROCESS | 25 | 7971.2 | 0.00 | 0.01 | 14 | 4365 | 0.02 | 0.11 | 11 | 3606.2 | 0.01 | 0.38 |
| GO_REGULATION_OF_INFLAMMATORY_RESPONSE | 41 | 13291 | 0.00 | 0.00 | 30 | 9480.9 | 0.00 | 0.02 | 11 | 3809.6 | 0.00 | 0.26 |
| GO_RESPONSE_TO_TOXIC_SUBSTANCE | 48 | 13391 | 0.01 | 0.04 | 23 | 6552.7 | 0.04 | 0.17 | 25 | 6838.1 | 0.02 | 0.42 |
| GO_CELLULAR_CHEMICAL_HOMEOSTASIS | 62 | 17171 | 0.00 | 0.02 | 42 | 12277 | 0.01 | 0.04 | 20 | 4894.2 | 0.16 | 0.71 |
| GO_POSITIVE_REGULATION_OF_TRANSPORT | 86 | 22281 | 0.01 | 0.09 | 64 | 16810 | 0.04 | 0.16 | 22 | 5471.6 | 0.12 | 0.66 |
| GO_REGULATION_OF_LIPID_CATABOLIC_PROCESS | 21 | 7420.9 | 0.00 | 0.00 | 12 | 4869.3 | 0.00 | 0.00 | 9 | 2551.6 | 0.09 | 0.59 |
| GO_REGULATION_OF_IMMUNE_EFFECTOR_PROCESS | 36 | 10243 | 0.01 | 0.07 | 24 | 6890.9 | 0.04 | 0.15 | 12 | 3351.6 | 0.07 | 0.56 |
| GO_LIPID_LOCALIZATION | 61 | 17939 | 0.00 | 0.00 | 41 | 13294 | 0.00 | 0.00 | 20 | 4644.4 | 0.26 | 0.83 |
| GO_REGULATION_OF_PROTEOLYSIS | 93 | 23839 | 0.01 | 0.09 | 61 | 17051 | 0.00 | 0.03 | 32 | 6788.6 | 0.47 | 0.97 |
| GO_POSITIVE_REGULATION_OF_SECRETION | 30 | 9267.7 | 0.00 | 0.01 | 26 | 8328.8 | 0.00 | 0.02 | 4 | 938.94 | 0.37 | 0.91 |
| GO_NEGATIVE_REGULATION_OF_RESPONSE_TO_WOUNDING | 27 | 8785.1 | 0.00 | 0.01 | 21 | 6818 | 0.00 | 0.03 | 6 | 1967.1 | 0.04 | 0.53 |
| GO_NEGATIVE_REGULATION_OF_MULTICELLULAR_ORGANISMAL_{—} | 90 | 23438 | 0.01 | 0.06 | 64 | 16486 | 0.07 | 0.23 | 26 | 6951.7 | 0.03 | 0.52 |
| PROCESS | | | | | | | | | | | | |
| GO_CELLULAR_HOMEOSTASIS | 79 | 21946 | 0.00 | 0.01 | 54 | 15425 | 0.00 | 0.03 | 25 | 6521.7 | 0.05 | 0.56 |
| GO_SINGLE_ORGANISM_CATABOLIC_PROCESS | 166 | 42191 | 0.00 | 0.03 | 83 | 23989 | 0.00 | 0.00 | 83 | 18202 | 0.28 | 0.84 |
| GO_RESPONSE_TO_OXIDATIVE_STRESS | 61 | 16457 | 0.01 | 0.06 | 39 | 10687 | 0.03 | 0.13 | 22 | 5770.5 | 0.06 | 0.56 |
| GO_BEHAVIOR | 31 | 8972 | 0.01 | 0.06 | 21 | 6660.3 | 0.01 | 0.04 | 10 | 2311.7 | 0.33 | 0.90 |
| GO_ACUTE_PHASE_RESPONSE | 21 | 7319.5 | 0.00 | 0.01 | 15 | 5604.1 | 0.00 | 0.00 | 6 | 1715.4 | 0.12 | 0.66 |
| GO_NEGATIVE_REGULATION_OF_RESPONSE_TO_EXTERNAL_{—} | 35 | 9962.4 | 0.01 | 0.07 | 27 | 7899.4 | 0.02 | 0.09 | 8 | 2063 | 0.20 | 0.75 |
| STIMULUS | | | | | | | | | | | | |
| GO_REGULATION_OF_APOPTOTIC_SIGNALING_PATHWAY | 54 | 15199 | 0.00 | 0.02 | 43 | 12306 | 0.01 | 0.04 | 11 | 2892.7 | 0.14 | 0.68 |
| GO_NEGATIVE_REGULATION_OF_CELL_PROLIFERATION | 48 | 14057 | 0.00 | 0.01 | 34 | 10182 | 0.00 | 0.03 | 14 | 3875.5 | 0.06 | 0.56 |
| GO_RESPONSE_TO_REACTIVE_OXYGEN_SPECIES | 36 | 10248 | 0.01 | 0.07 | 23 | 6148.8 | 0.12 | 0.34 | 13 | 4099.2 | 0.01 | 0.38 |
| GO_ENDOCYTIC_VESICLE | 25 | 7350.4 | 0.01 | 0.08 | 19 | 5246.5 | 0.09 | 0.29 | 6 | 2103.9 | 0.02 | 0.40 |
| GO_ENDOPLASMIC_RETICULUM_PART | 168 | 42254 | 0.00 | 0.04 | 119 | 31832 | 0.00 | 0.02 | 49 | 10422 | 0.46 | 0.97 |
| GO_ENDOPLASMIC_RETICULUM_LUMEN | 36 | 10132 | 0.01 | 0.09 | 28 | 8391.3 | 0.01 | 0.05 | 8 | 1740.3 | 0.44 | 0.95 |
| GO_ENDOPLASMIC_RETICULUM | 214 | 53290 | 0.00 | 0.03 | 152 | 39291 | 0.01 | 0.04 | 62 | 13999 | 0.21 | 0.75 |
| GO_LIPID_TRANSPORTER_ACTIVITY | 27 | 9133.9 | 0.00 | 0.00 | 17 | 6244.4 | 0.00 | 0.00 | 10 | 2889.5 | 0.06 | 0.56 |
| GO_SULFUR_COMPOUND_BINDING | 36 | 12559 | 0.00 | 0.00 | 23 | 8121.4 | 0.00 | 0.00 | 13 | 4437.8 | 0.00 | 0.26 |
| GO_STEROID_BINDING | 20 | 6566.6 | 0.00 | 0.01 | 10 | 3560.3 | 0.01 | 0.04 | 10 | 3006.3 | 0.04 | 0.53 |
| GO_GLYCOSAMINOGLYCAN_BINDING | 20 | 6655.2 | 0.00 | 0.01 | 17 | 5696.2 | 0.00 | 0.03 | 3 | 959.02 | 0.14 | 0.68 |
| GO_ALCOHOL_BINDING | 20 | 7130.5 | 0.00 | 0.01 | 10 | 3920.7 | 0.00 | 0.01 | 10 | 3209.7 | 0.01 | 0.40 |
| GO_CARBOXYLIC_ESTER_HYDROLASE_ACTIVITY | 21 | 6241.5 | 0.02 | 0.10 | 12 | 4474.4 | 0.00 | 0.01 | 9 | 1767.1 | 0.60 | 1.00 |
| GO_LIPID_BINDING | 81 | 21990 | 0.00 | 0.03 | 53 | 14919 | 0.01 | 0.04 | 28 | 7070.2 | 0.07 | 0.56 |
| GO_RECEPTOR_BINDING | 144 | 38089 | 0.00 | 0.00 | 105 | 28493 | 0.00 | 0.02 | 39 | 9595.4 | 0.07 | 0.56 |
| GO_COENZYME_BINDING | 46 | 13083 | 0.00 | 0.03 | 27 | 8640.9 | 0.00 | 0.02 | 19 | 4442.1 | 0.26 | 0.83 |
| HALLMARK_ADIPOGENESIS | 66 | 18461 | 0.00 | 0.01 | 35 | 11301 | 0.00 | 0.00 | 31 | 7160 | 0.22 | 0.80 |
| HALLMARK_XENOBIOTIC_METABOLISM | 79 | 25124 | 0.00 | 0.00 | 45 | 16165 | 0.00 | 0.00 | 34 | 8958.6 | 0.02 | 0.58 |
| HALLMARK_FATTY_ACID_METABOLISM | 56 | 14864 | 0.02 | 0.06 | 29 | 9719.9 | 0.00 | 0.00 | 27 | 5144.5 | 0.75 | 0.99 |
| HALLMARK_COAGULATION | 44 | 12802 | 0.00 | 0.01 | 32 | 9497 | 0.01 | 0.02 | 12 | 3304.8 | 0.08 | 0.80 |
| HALLMARK_BILE_ACID_METABOLISM | 47 | 13258 | 0.00 | 0.02 | 21 | 7236 | 0.00 | 0.00 | 26 | 6022.1 | 0.24 | 0.80 |
| HALLMARK_PEROXISOME | 38 | 10985 | 0.01 | 0.03 | 20 | 6649.6 | 0.00 | 0.01 | 18 | 4335.2 | 0.20 | 0.80 |

TABLE 10
Pathway enrichments unique to PH.

| | Genes. | Stat.non. | p.non. | padj.non. | Genes. | Stat.mix. | p.mix. | padj.mix. | Genes. | Stat.mix. | p.mix. | padj.mix. |
|---|---|---|---|---|---|---|---|---|---|---|---|---|
| | tot._{—} | dir.._{—} | dir.._{—} | dir.._{—} | up._{—} | dir.up._{—} | dir.up._{—} | dir.up._{—} | down._{—} | dir.dn._{—} | dir.dn._{—} | dir.dn._{—} |
| | APAP | APAP | APAP | APAP | APAP | APAP | APAP | APAP | APAP | APAP | APAP | APAP |
| SENESE_HDAC3_TARGETS_UP | 75 | 13473 | 0.99 | 1.00 | 70 | 12747 | 1.00 | 1.00 | 5 | 725.8 | 0.82 | 1.00 |
| BUYTAERT_PHOTODYNAMIC_THERAPY_STRESS_UP | 102 | 21480 | 0.77 | 1.00 | 90 | 18877 | 0.90 | 1.00 | 12 | 2603.8 | 0.42 | 1.00 |
| GERY_CEBP_TARGETS | 28 | 7584.2 | 0.05 | 0.38 | 22 | 6232.4 | 0.05 | 0.33 | 6 | 1351.8 | 0.40 | 1.00 |
| FOSTER_TOLERANT_MACROPHAGE_DN | 48 | 10226 | 0.65 | 1.00 | 45 | 9539.9 | 0.77 | 1.00 | 3 | 686.22 | 0.39 | 1.00 |
| BLUM_RESPONSE_TO_SALIRASIB_UP | 53 | 12422 | 0.28 | 0.86 | 50 | 11521 | 0.47 | 0.99 | 3 | 901.04 | 0.18 | 1.00 |
| WONG_ADULT_TISSUE_STEM_MODULE | 83 | 19476 | 0.22 | 0.79 | 63 | 14741 | 0.41 | 0.92 | 20 | 4734.8 | 0.22 | 1.00 |
| PILON_KLF1_TARGETS_UP | 93 | 23146 | 0.04 | 0.35 | 72 | 19825 | 0.00 | 0.07 | 21 | 3321 | 0.94 | 1.00 |
| GO_ANATOMICAL_STRUCTURE_FORMATION_INVOLVED_IN_{—} | 56 | 13042 | 0.29 | 0.66 | 49 | 12197 | 0.19 | 0.45 | 7 | 845.45 | 0.94 | 1.00 |
| MORPHOGENESIS | | | | | | | | | | | | |
| GO_CIRCULATORY_SYSTEM_PROCESS | 35 | 9196.8 | 0.06 | 0.24 | 24 | 6528.3 | 0.09 | 0.28 | 11 | 2668.5 | 0.25 | 0.82 |
| GO_CELLULAR_RESPONSE_TO_EXTERNAL_STIMULUS | 37 | 9083.2 | 0.17 | 0.48 | 32 | 7625.3 | 0.37 | 0.70 | 5 | 1457.8 | 0.13 | 0.66 |
| GO_RESPONSE_TO_WOUNDING | 69 | 17868 | 0.02 | 0.11 | 59 | 15896 | 0.02 | 0.11 | 10 | 1971.7 | 0.60 | 1.00 |
| GO_CELLULAR_RESPONSE_TO_EXTRACELLULAR_STIMULUS | 25 | 6176.3 | 0.21 | 0.53 | 20 | 4718.4 | 0.42 | 0.77 | 5 | 1457.8 | 0.13 | 0.66 |
| GO_CELL_ACTIVATION | 41 | 9934.8 | 0.19 | 0.52 | 37 | 8671.2 | 0.43 | 0.78 | 4 | 1263.6 | 0.09 | 0.61 |
| GO_CELLULAR_RESPONSE_TO_OXYGEN_CONTAINING_COMPOUND | 102 | 21984 | 0.66 | 1.00 | 71 | 15558 | 0.72 | 1.00 | 31 | 6426.1 | 0.53 | 1.00 |
| GO_VESICLE_MEDIATED_TRANSPORT | 129 | 29919 | 0.22 | 0.55 | 111 | 26242 | 0.30 | 0.61 | 18 | 3676.9 | 0.56 | 1.00 |
| GO_ENZYME_LINKED_RECEPTOR_PROTEIN_SIGNALING_PATHWAY | 60 | 11703 | 0.91 | 1.00 | 50 | 9424.2 | 0.97 | 1.00 | 10 | 2278.4 | 0.36 | 0.91 |
| GO_RESPONSE_TO_BACTERIUM | 54 | 14413 | 0.02 | 0.10 | 36 | 9569.4 | 0.08 | 0.27 | 18 | 4843.7 | 0.06 | 0.56 |
| GO_REGULATION_OF_CATABOLIC_PROCESS | 91 | 23179 | 0.02 | 0.12 | 61 | 16224 | 0.03 | 0.13 | 30 | 6954.8 | 0.22 | 0.80 |
| GO_RESPONSE_TO_KETONE | 36 | 9267.4 | 0.09 | 0.31 | 28 | 8068.1 | 0.02 | 0.11 | 8 | 1199.3 | 0.86 | 1.00 |
| GO_POSITIVE_REGULATION_OF_CELL_ADHESION | 29 | 7617.2 | 0.08 | 0.29 | 25 | 6568.2 | 0.14 | 0.36 | 4 | 1049.1 | 0.25 | 0.83 |
| GO_RESPONSE_TO_HORMONE | 130 | 31935 | 0.03 | 0.17 | 92 | 24993 | 0.00 | 0.03 | 38 | 6942.1 | 0.87 | 1.00 |
| GO_BLOOD_VESSEL_MORPHOGENESIS | 35 | 9233.1 | 0.06 | 0.23 | 29 | 7973.1 | 0.06 | 0.22 | 6 | 1260.1 | 0.49 | 0.98 |
| GO_RESPONSE_TO_ESTROGEN | 31 | 8617.4 | 0.02 | 0.13 | 21 | 6012.8 | 0.05 | 0.19 | 10 | 2604.5 | 0.16 | 0.71 |
| GO_RESPONSE_TO_RADIATION | 29 | 5711.7 | 0.80 | 1.00 | 23 | 4585.5 | 0.83 | 1.00 | 6 | 1126.1 | 0.63 | 1.00 |
| GO_RESPONSE_TO_EXTRACELLULAR_STIMULUS | 71 | 17910 | 0.05 | 0.22 | 44 | 11428 | 0.10 | 0.29 | 27 | 6481.9 | 0.16 | 0.71 |
| GO_CELLULAR_RESPONSE_TO_NITROGEN_COMPOUND | 57 | 11255 | 0.89 | 1.00 | 44 | 8785.4 | 0.90 | 1.00 | 13 | 2469.4 | 0.68 | 1.00 |
| GO_NEGATIVE_REGULATION_OF_CATALYTIC_ACTIVITY | 104 | 25690 | 0.04 | 0.19 | 77 | 19130 | 0.13 | 0.35 | 27 | 6560 | 0.13 | 0.67 |
| GO_VASCULATURE_DEVELOPMENT | 39 | 9667.3 | 0.14 | 0.42 | 33 | 8407.2 | 0.17 | 0.43 | 6 | 1260.1 | 0.49 | 0.98 |
| GO_RESPONSE_TO_ABIOTIC_STIMULUS | 93 | 22945 | 0.05 | 0.22 | 69 | 17712 | 0.07 | 0.24 | 24 | 5233.9 | 0.40 | 0.94 |
| GO_RESPONSE_TO_DRUG | 72 | 17868 | 0.07 | 0.27 | 43 | 11067 | 0.12 | 0.33 | 29 | 6800.6 | 0.19 | 0.74 |
| GO_RESPONSE_TO_GROWTH_FACTOR | 39 | 7982.8 | 0.75 | 1.00 | 32 | 6574.3 | 0.81 | 1.00 | 7 | 1408.5 | 0.55 | 1.00 |
| GO_NEGATIVE_REGULATION_OF_PROTEIN_METABOLIC_PROCESS | 120 | 27553 | 0.27 | 0.63 | 90 | 22010 | 0.17 | 0.42 | 30 | 5542.5 | 0.82 | 1.00 |
| GO_TRANSMEMBRANE_RECEPTOR_PROTEIN_TYROSINE_KINASE_{—} | 46 | 8645.1 | 0.93 | 1.00 | 40 | 7347.9 | 0.97 | 1.00 | 6 | 1297.2 | 0.46 | 0.97 |
| SIGNALING_PATHWAY | | | | | | | | | | | | |
| GO_CELLULAR_RESPONSE_TO_PEPTIDE | 39 | 7740.3 | 0.82 | 1.00 | 31 | 6406.6 | 0.79 | 1.00 | 8 | 1333.8 | 0.78 | 1.00 |
| GO_HEXOSE_METABOLIC_PROCESS | 23 | 5891.2 | 0.15 | 0.43 | 11 | 3367.8 | 0.05 | 0.20 | 12 | 2523.3 | 0.48 | 0.98 |
| GO_CELLULAR_RESPONSE_TO_STRESS | 149 | 32891 | 0.53 | 0.97 | 115 | 24498 | 0.88 | 1.00 | 34 | 8393.2 | 0.08 | 0.58 |
| GO_ENDOCYTOSIS | 55 | 12969 | 0.25 | 0.60 | 45 | 10610 | 0.39 | 0.74 | 10 | 2358.7 | 0.30 | 0.86 |
| GO_CIRCULATORY_SYSTEM_DEVELOPMENT | 56 | 12966 | 0.31 | 0.69 | 45 | 11136 | 0.22 | 0.51 | 11 | 1830.1 | 0.83 | 1.00 |
| GO_RESPONSE_TO_STARVATION | 26 | 6430.6 | 0.19 | 0.52 | 19 | 5300.9 | 0.08 | 0.27 | 7 | 1129.7 | 0.79 | 1.00 |
| GO_HEMOSTASIS | 38 | 10217 | 0.03 | 0.17 | 33 | 9397.9 | 0.02 | 0.10 | 5 | 819.47 | 0.73 | 1.00 |
| GO_RESPONSE_TO_MOLECULE_OF_BACTERIAL_ORIGIN | 38 | 9958.5 | 0.06 | 0.23 | 26 | 6585.1 | 0.21 | 0.50 | 12 | 3373.4 | 0.06 | 0.56 |
| NA | NA | NA | NA | NA | NA | NA | NA | NA | NA | NA | NA | NA |
| GO_CELL_SURFACE | 63 | 15190 | 0.15 | 0.45 | 55 | 14046 | 0.10 | 0.31 | 8 | 1143.9 | 0.89 | 1.00 |
| GO_PEPTIDASE_REGULATOR_ACTIVITY | 32 | 8831.6 | 0.02 | 0.13 | 24 | 7480.4 | 0.01 | 0.04 | 8 | 1351.2 | 0.76 | 1.00 |
| NA.1 | NA | NA | NA | NA | NA | NA | NA | NA | NA | NA | NA | NA |
| GO_MOLECULAR_FUNCTION_REGULATOR | 109 | 27456 | 0.02 | 0.11 | 84 | 20954 | 0.11 | 0.32 | 25 | 6501.3 | 0.05 | 0.56 |
| GO_PEPTIDASE_INHIBITOR_ACTIVITY | 24 | 6956.3 | 0.02 | 0.11 | 19 | 6368.7 | 0.00 | 0.02 | 5 | 587.56 | 0.91 | 1.00 |
| GO_PHOSPHOLIPID_BINDING | 39 | 9728.5 | 0.13 | 0.41 | 28 | 6245.1 | 0.58 | 0.94 | 11 | 3483.4 | 0.02 | 0.40 |
| HALLMARK_TNFA_SIGNALING_VIA_NFKB | 41 | 9591.7 | 0.30 | 0.43 | 38 | 9112.1 | 0.34 | 0.48 | 3 | 479.62 | 0.69 | 0.99 |
| | Genes. | Stat.non. | p.non. | padj.non. | Genes. | Stat.mix. | p.mix. | padj.mix. | Genes. | Stat.mix. | p.mix. | padj.mix. |
| | tot._{—} | dir.._{—} | dir.._{—} | dir.._{—} | up._{—} | dir.up._{—} | dir.up._{—} | dir.up._{—} | down._{—} | dir.dn._{—} | dir.dn._{—} | dir.dn._{—} |
| | PH | PH | PH | PH | PH | PH | PH | PH | PH | PH | PH | PH |
| SENESE_HDAC3_TARGETS_UP | 32 | 13300 | 0.00 | 0.06 | 29 | 12340 | 0.00 | 0.15 | 3 | 960.52 | 0.53 | 1.00 |
| BUYTAERT_PHOTODYNAMIC_THERAPY_STRESS_UP | 50 | 20022 | 0.00 | 0.06 | 44 | 18582 | 0.00 | 0.12 | 6 | 1440 | 0.92 | 1.00 |
| GERY_CEBP_TARGETS | 30 | 12477 | 0.00 | 0.08 | 27 | 11455 | 0.01 | 0.18 | 3 | 1022.1 | 0.45 | 1.00 |
| FOSTER_TOLERANT_MACROPHAGE_DN | 27 | 11513 | 0.00 | 0.05 | 25 | 10728 | 0.00 | 0.15 | 2 | 785.41 | 0.27 | 1.00 |
| BLUM_RESPONSE_TO_SALIRASIB_UP | 20 | 8679.2 | 0.00 | 0.06 | 17 | 7593.1 | 0.00 | 0.14 | 3 | 1086.1 | 0.30 | 1.00 |
| WONG_ADULT_TISSUE_STEM_MODULE | 61 | 24461 | 0.00 | 0.05 | 51 | 21066 | 0.00 | 0.15 | 10 | 3395.8 | 0.34 | 1.00 |
| PILON_KLF1_TARGETS_UP | 51 | 20388 | 0.00 | 0.08 | 36 | 14725 | 0.02 | 0.28 | 15 | 5662.9 | 0.05 | 1.00 |
| GO_ANATOMICAL_STRUCTURE_FORMATION_INVOLVED_IN_{—} | 34 | 13552 | 0.01 | 0.06 | 29 | 11738 | 0.06 | 0.28 | 5 | 1813.7 | 0.26 | 1.00 |
| MORPHOGENESIS | | | | | | | | | | | | |
| GO_CIRCULATORY_SYSTEM_PROCESS | 22 | 8978.4 | 0.01 | 0.08 | 17 | 6980.5 | 0.08 | 0.34 | 5 | 1997.9 | 0.10 | 0.80 |
| GO_CELLULAR_RESPONSE_TO_EXTERNAL_STIMULUS | 28 | 11316 | 0.01 | 0.07 | 24 | 10140 | 0.01 | 0.21 | 4 | 1175.3 | 0.67 | 1.00 |
| GO_RESPONSE_TO_WOUNDING | 51 | 20277 | 0.00 | 0.03 | 42 | 17126 | 0.02 | 0.21 | 9 | 3150.7 | 0.27 | 1.00 |
| GO_CELLULAR_RESPONSE_TO_EXTRACELLULAR_STIMULUS | 23 | 9751.4 | 0.00 | 0.03 | 20 | 8698.6 | 0.00 | 0.18 | 3 | 1052.7 | 0.39 | 1.00 |
| GO_CELL_ACTIVATION | 27 | 11250 | 0.00 | 0.03 | 24 | 10335 | 0.00 | 0.18 | 3 | 915.54 | 0.59 | 1.00 |
| GO_CELLULAR_RESPONSE_TO_OXYGEN_CONTAINING_COMPOUND | 56 | 21925 | 0.00 | 0.05 | 41 | 16778 | 0.02 | 0.21 | 15 | 5147.1 | 0.27 | 1.00 |
| GO_VESICLE_MEDIATED_TRANSPORT | 82 | 31632 | 0.00 | 0.04 | 70 | 27402 | 0.07 | 0.31 | 12 | 4230.5 | 0.22 | 1.00 |
| GO_ENZYME_LINKED_RECEPTOR_PROTEIN_SIGNALING_PATHWAY | 32 | 13199 | 0.00 | 0.03 | 27 | 11210 | 0.02 | 0.21 | 5 | 1989.9 | 0.10 | 0.80 |
| GO_RESPONSE_TO_BACTERIUM | 36 | 14568 | 0.00 | 0.04 | 28 | 11501 | 0.03 | 0.24 | 8 | 3067.3 | 0.10 | 0.80 |
| GO_REGULATION_OF_CATABOLIC_PROCESS | 54 | 20833 | 0.01 | 0.10 | 33 | 13205 | 0.07 | 0.31 | 21 | 7628.7 | 0.08 | 0.74 |
| GO_RESPONSE_TO_KETONE | 21 | 8843 | 0.00 | 0.04 | 18 | 7837.3 | 0.01 | 0.20 | 3 | 1005.7 | 0.48 | 1.00 |
| GO_POSITIVE_REGULATION_OF_CELL_ADHESION | 21 | 8588.5 | 0.01 | 0.09 | 19 | 7667.5 | 0.12 | 0.39 | 2 | 921.03 | 0.14 | 0.88 |
| GO_RESPONSE_TO_HORMONE | 79 | 29962 | 0.01 | 0.09 | 55 | 22279 | 0.01 | 0.21 | 24 | 7683.7 | 0.53 | 1.00 |
| GO_BLOOD_VESSEL_MORPHOGENESIS | 30 | 12059 | 0.01 | 0.08 | 25 | 10245 | 0.05 | 0.28 | 5 | 1813.7 | 0.26 | 1.00 |
| GO_RESPONSE_TO_ESTROGEN | 20 | 8429.9 | 0.00 | 0.04 | 14 | 5989.2 | 0.03 | 0.23 | 6 | 2440.7 | 0.06 | 0.68 |
| GO_RESPONSE_TO_RADIATION | 21 | 9009 | 0.00 | 0.03 | 19 | 8327.5 | 0.00 | 0.15 | 2 | 681.51 | 0.40 | 1.00 |
| GO_RESPONSE_TO_EXTRACELLULAR_STIMULUS | 58 | 22855 | 0.00 | 0.03 | 38 | 15652 | 0.01 | 0.21 | 20 | 7204 | 0.10 | 0.80 |
| GO_CELLULAR_RESPONSE_TO_NITROGEN_COMPOUND | 28 | 11402 | 0.01 | 0.05 | 22 | 9147.5 | 0.03 | 0.24 | 6 | 2254.5 | 0.16 | 0.92 |
| GO_NEGATIVE_REGULATION_OF_CATALYTIC_ACTIVITY | 61 | 23692 | 0.01 | 0.05 | 39 | 15895 | 0.02 | 0.21 | 22 | 7797.4 | 0.12 | 0.85 |
| GO_VASCULATURE_DEVELOPMENT | 32 | 12980 | 0.00 | 0.04 | 27 | 11166 | 0.02 | 0.22 | 5 | 1813.7 | 0.26 | 1.00 |
| GO_RESPONSE_TO_ABIOTIC_STIMULUS | 63 | 25227 | 0.00 | 0.02 | 48 | 19967 | 0.00 | 0.14 | 15 | 5260.1 | 0.20 | 0.98 |
| GO_RESPONSE_TO_DRUG | 47 | 18305 | 0.01 | 0.09 | 29 | 12241 | 0.00 | 0.19 | 18 | 6064 | 0.31 | 1.00 |
| GO_RESPONSE_TO_GROWTH_FACTOR | 23 | 9758.3 | 0.00 | 0.03 | 18 | 8031.8 | 0.00 | 0.14 | 5 | 1726.5 | 0.36 | 1.00 |
| GO_NEGATIVE_REGULATION_OF_PROTEIN_METABOLIC_PROCESS | 73 | 28292 | 0.00 | 0.04 | 52 | 21388 | 0.01 | 0.19 | 21 | 6904.6 | 0.41 | 1.00 |
| GO_TRANSMEMBRANE_RECEPTOR_PROTEIN_TYROSINE_KINASE_{—} | 23 | 9498.8 | 0.01 | 0.05 | 19 | 7969.4 | 0.03 | 0.24 | 4 | 1529.4 | 0.19 | 0.98 |
| SIGNALING_PATHWAY | | | | | | | | | | | | |
| GO_CELLULAR_RESPONSE_TO_PEPTIDE | 21 | 8753.6 | 0.01 | 0.05 | 18 | 7556.8 | 0.03 | 0.24 | 3 | 1196.7 | 0.17 | 0.95 |
| GO_HEXOSE_METABOLIC_PROCESS | 21 | 8616.8 | 0.01 | 0.08 | 13 | 5865.8 | 0.00 | 0.14 | 8 | 2751 | 0.33 | 1.00 |
| GO_CELLULAR_RESPONSE_TO_STRESS | 87 | 33202 | 0.01 | 0.05 | 63 | 25236 | 0.02 | 0.21 | 24 | 7965.7 | 0.36 | 1.00 |
| GO_ENDOCYTOSIS | 44 | 17555 | 0.00 | 0.04 | 39 | 15854 | 0.02 | 0.23 | 5 | 1701.1 | 0.39 | 1.00 |
| GO_CIRCULATORY_SYSTEM_DEVELOPMENT | 41 | 16113 | 0.01 | 0.08 | 34 | 13665 | 0.06 | 0.28 | 7 | 2448.2 | 0.31 | 1.00 |
| GO_RESPONSE_TO_STARVATION | 26 | 10695 | 0.00 | 0.05 | 22 | 9438.5 | 0.01 | 0.21 | 4 | 1256.2 | 0.54 | 1.00 |
| GO_HEMOSTASIS | 27 | 11311 | 0.00 | 0.03 | 24 | 10276 | 0.00 | 0.19 | 3 | 1035.4 | 0.43 | 1.00 |
| GO_RESPONSE_TO_MOLECULE_OF_BACTERIAL_ORIGIN | 28 | 11380 | 0.01 | 0.06 | 22 | 9029.7 | 0.06 | 0.28 | 6 | 2349.8 | 0.10 | 0.80 |
| NA | 20 | 8636.2 | 0.00 | 0.03 | 19 | 8175.6 | 0.01 | 0.21 | 1 | 460.52 | 0.38 | 1.00 |
| GO_CELL_SURFACE | 42 | 16481 | 0.01 | 0.08 | 37 | 14367 | 0.20 | 0.49 | 5 | 2114.3 | 0.04 | 0.60 |
| GO_PEPTIDASE_REGULATOR_ACTIVITY | 26 | 10611 | 0.01 | 0.06 | 20 | 8341.2 | 0.04 | 0.25 | 6 | 2269.3 | 0.15 | 0.91 |
| NA.1 | 20 | 8196.3 | 0.01 | 0.09 | 14 | 5686.3 | 0.14 | 0.43 | 6 | 2509.9 | 0.03 | 0.48 |
| GO_MOLECULAR_FUNCTION_REGULATOR | 77 | 30265 | 0.00 | 0.02 | 57 | 22685 | 0.04 | 0.25 | 20 | 7579.9 | 0.03 | 0.48 |
| GO_PEPTIDASE_INHIBITOR_ACTIVITY | 22 | 9121.8 | 0.01 | 0.05 | 19 | 7880.7 | 0.05 | 0.28 | 3 | 1241.1 | 0.14 | 0.88 |
| GO_PHOSPHOLIPID_BINDING | 21 | 8989.6 | 0.00 | 0.03 | 12 | 4844.9 | 0.19 | 0.47 | 9 | 4144.7 | 0.00 | 0.05 |
| HALLMARK_TNFA_SIGNALING_VIA_NFKB | 37 | 14438 | 0.02 | 0.10 | 33 | 13859 | 0.00 | 0.08 | 4 | 579.08 | 1.00 | 1.00 |

TABLE 11
Pathway enrichments shared between APAP and PH.

| | Genes. | Stat.non. | p.non. | padj.non. | Genes. | Stat.mix. | p.mix. | padj.mix. | Genes. | Stat.mix. | p.mix. | padj.mix. |
|---|---|---|---|---|---|---|---|---|---|---|---|---|
| | tot._{—} | dir.._{—} | dir.._{—} | dir.._{—} | up._{—} | dir.up._{—} | dir.up._{—} | dir.up._{—} | down._{—} | dir.dn._{—} | dir.dn._{—} | dir.dn._{—} |
| | APAP | APAP | APAP | APAP | APAP | APAP | APAP | APAP | APAP | APAP | APAP | APAP |
| GO_NEGATIVE_REGULATION_OF_PROTEOLYSIS | 4.3 | 12343 | 0.00 | 0.03 | 34 | 10537 | 0.00 | 0.01 | 9 | 1806.5 | 0.56 | 1.00 |
| GO_CHEMICAL_HOMEOSTASIS | 112 | 30618 | 0.00 | 0.01 | 77 | 22096 | 0.00 | 0.01 | 35 | 8522.2 | 0.10 | 0.63 |
| GO_SECRETION_BY_CELL | 46 | 13917 | 0.00 | 0.01 | 41 | 12965 | 0.00 | 0.00 | 5 | 952.32 | 0.59 | 1.00 |
| GO_REGULATION_OF_HYDROLASE_ACTIVITY | 118 | 30643 | 0.00 | 0.03 | 92 | 24535 | 0.01 | 0.05 | 26 | 6108.3 | 0.20 | 0.75 |
| GO_REGULATION_OF_BODY_FLUID_LEVELS | 49 | 14206 | 0.00 | 0.01 | 41 | 12431 | 0.00 | 0.01 | 8 | 1775 | 0.41 | 0.94 |
| GO_HOMEOSTATIC_PROCESS | 150 | 40881 | 0.00 | 0.00 | 105 | 29582 | 0.00 | 0.00 | 45 | 11298 | 0.04 | 0.53 |
| GO_WOUND_HEALING | 58 | 16314 | 0.00 | 0.02 | 51 | 14915 | 0.00 | 0.02 | 7 | 1398.7 | 0.56 | 1.00 |
| GO_GLYCEROLIPID_METABOLIC_PROCESS | 59 | 17149 | 0.00 | 0.01 | 40 | 11310 | 0.02 | 0.09 | 19 | 5839.1 | 0.00 | 0.26 |
| GO_RESPONSE_TO_EXTERNAL_STIMULUS | 166 | 41066 | 0.01 | 0.08 | 113 | 28480 | 0.05 | 0.19 | 53 | 12586 | 0.10 | 0.63 |
| GO_RESPONSE_TO_OXYGEN_CONTAINING_COMPOUND | 186 | 45695 | 0.01 | 0.09 | 124 | 32182 | 0.01 | 0.05 | 62 | 13512 | 0.34 | 0.90 |
| GO_RESPONSE_TO_LIPID | 120 | 30247 | 0.01 | 0.09 | 82 | 22400 | 0.00 | 0.03 | 38 | 7846.9 | 0.56 | 1.00 |
| GO_NEUTRAL_LIPID_METABOLIC_PROCESS | 34 | 10847 | 0.00 | 0.01 | 20 | 6084.9 | 0.02 | 0.09 | 14 | 4762.1 | 0.00 | 0.26 |
| GO_NEGATIVE_REGULATION_OF_HYDROLASE_ACTIVITY | 57 | 16248 | 0.00 | 0.01 | 43 | 12401 | 0.00 | 0.04 | 14 | 3847.1 | 0.07 | 0.56 |
| GO_ION_HOMEOSTASIS | 65 | 17747 | 0.00 | 0.04 | 47 | 13484 | 0.01 | 0.04 | 18 | 4262.7 | 0.23 | 0.80 |
| GO_RESPONSE_TO_BIOTIC_STIMULUS | 83 | 21979 | 0.01 | 0.05 | 60 | 15941 | 0.03 | 0.13 | 23 | 6037.9 | 0.05 | 0.56 |
| GO_EXOCYTOSIS | 36 | 10787 | 0.00 | 0.02 | 32 | 10043 | 0.00 | 0.01 | 4 | 743.54 | 0.59 | 1.00 |
| GO_PLATELET_DEGRANULATION | 31 | 10055 | 0.00 | 0.01 | 28 | 9340.2 | 0.00 | 0.01 | 3 | 714.91 | 0.36 | 0.91 |
| GO_RESPONSE_TO_ALCOHOL | 65 | 17315 | 0.01 | 0.07 | 48 | 13614 | 0.01 | 0.04 | 17 | 3701.2 | 0.42 | 0.94 |
| GO_REGULATED_EXOCYTOSIS | 33 | 10189 | 0.00 | 0.01 | 30 | 9473.9 | 0.00 | 0.02 | 3 | 714.91 | 0.36 | 0.91 |
| GO_NEGATIVE_REGULATION_OF_PEPTIDASE_ACTIVITY | 37 | 10915 | 0.00 | 0.03 | 28 | 9108.3 | 0.00 | 0.01 | 9 | 1806.5 | 0.56 | 1.00 |
| GO_EXTRACELLULAR_MATRIX | 29 | 8933.1 | 0.00 | 0.02 | 24 | 7347.9 | 0.01 | 0.05 | 5 | 1585.2 | 0.07 | 0.56 |
| GO_SECRETORY_GRANULE | 38 | 11617 | 0.00 | 0.01 | 33 | 10766 | 0.00 | 0.01 | 5 | 851.27 | 0.70 | 1.00 |
| GO_PLATELET_ALPHA_GRANULE | 20 | 6903.9 | 0.00 | 0.01 | 20 | 6903.9 | 0.00 | 0.01 | 0 | NA | NA | NA |
| GO_SECRETORY_GRANULE_LUMEN | 25 | 8665.9 | 0.00 | 0.01 | 23 | 8340.8 | 0.00 | 0.00 | 2 | 325.07 | 0.62 | 1.00 |
| GO_SECRETORY_VESICLE | 46 | 12933 | 0.00 | 0.04 | 39 | 11952 | 0.00 | 0.01 | 7 | 980.73 | 0.88 | 1.00 |
| GO_VESICLE_LUMEN | 34 | 12292 | 0.00 | 0.00 | 29 | 10917 | 0.00 | 0.00 | 5 | 1375.6 | 0.18 | 0.74 |
| GO_BLOOD_MICROPARTICLE | 45 | 15877 | 0.00 | 0.00 | 37 | 13416 | 0.00 | 0.00 | 8 | 2460.8 | 0.05 | 0.55 |
| GO_INTRACELLULAR_VESICLE | 134 | 33750 | 0.01 | 0.07 | 109 | 27946 | 0.03 | 0.12 | 25 | 5804.5 | 0.24 | 0.81 |
| GO_EXTRACELLULAR_SPACE | 158 | 47392 | 0.00 | 0.00 | 111 | 35581 | 0.00 | 0.00 | 47 | 11811 | 0.03 | 0.52 |
| GO_CYTOPLASMIC_VESICLE_PART | 70 | 19517 | 0.00 | 0.01 | 60 | 16840 | 0.00 | 0.04 | 10 | 2676.2 | 0.12 | 0.66 |
| GO_PROTEIN_LIPID_COMPLEX | 22 | 7615.8 | 0.00 | 0.01 | 11 | 4064.5 | 0.00 | 0.01 | 11 | 3551.4 | 0.01 | 0.38 |
| GO_ENZYME_INHIBITOR_ACTIVITY | 46 | 13024 | 0.00 | 0.03 | 36 | 10396 | 0.01 | 0.06 | 10 | 2628.1 | 0.14 | 0.68 |
| GO_ENZYME_REGULATOR_ACTIVIIY | 92 | 24477 | 0.00 | 0.03 | 70 | 18618 | 0.02 | 0.11 | 22 | 5858.4 | 0.05 | 0.55 |
| HALLMARK_HYPOχIA | 31 | 8762.2 | 0.02 | 0.06 | 25 | 7882 | 0.00 | 0.01 | 6 | 880.21 | 0.83 | 0.99 |
| HALLMARK_APOPTOSIS | 33 | 9090.3 | 0.02 | 0.07 | 25 | 6879.2 | 0.07 | 0.15 | 8 | 2211.1 | 0.12 | 0.80 |
| HALLMARK_COMPLEMENT | 44 | 12175 | 0.01 | 0.04 | 36 | 10358 | 0.01 | 0.03 | 8 | 1816.6 | 0.37 | 0.84 |
| | Genes. | Stat.non. | p.non. | padj.non. | Genes. | Stat.mix. | p.mix. | padj.mix. | Genes. | Stat.mix. | p.mix. | padj.mix. |
| | tot._{—} | dir.._{—} | dir.._{—} | dir.._{—} | up._{—} | dir.up._{—} | dir.up._{—} | dir.up._{—} | down._{—} | dir.dn._{—} | dir.dn._{—} | dir.dn._{—} |
| | PH | PH | PH | PH | PH | PH | PH | PH | PH | PH | PH | PH |
| GO_NEGATIVE_REGULATION_OF_PROTEOLYSIS | 32 | 13244 | 0.00 | 0.03 | 26 | 10935 | 0.01 | 0.21 | 6 | 2308.3 | 0.13 | 0.85 |
| GO_CHEMICAL_HOMEOSTASIS | 70 | 26876 | 0.01 | 0.06 | 48 | 18932 | 0.09 | 0.34 | 22 | 7944.6 | 0.08 | 0.75 |
| GO_SECRETION_BY_CELL | 35 | 14350 | 0.00 | 0.03 | 28 | 11419 | 0.05 | 0.27 | 7 | 2930.8 | 0.03 | 0.48 |
| GO_REGULATION_OF_HYDROLASE_ACTIVITY | 79 | 31167 | 0.00 | 0.02 | 58 | 23315 | 0.02 | 0.21 | 21 | 7852 | 0.03 | 0.48 |
| GO_REGULATION_OF_BODY_FLUID_LEVELS | 38 | 15254 | 0.00 | 0.04 | 32 | 13261 | 0.01 | 0.21 | 6 | 1993.5 | 0.43 | 1.00 |
| GO_HOMEOSTATIC_PROCESS | 87 | 33210 | 0.01 | 0.05 | 55 | 21855 | 0.05 | 0.27 | 32 | 11356 | 0.08 | 0.74 |
| GO_WOUND_HEALING | 39 | 15745 | 0.00 | 0.03 | 33 | 13681 | 0.01 | 0.21 | 6 | 2063.7 | 0.35 | 1.00 |
| GO_GLYCEROLIPID_METABOLIC_PROCESS | 38 | 15218 | 0.00 | 0.04 | 22 | 8957.4 | 0.08 | 0.31 | 16 | 6260.9 | 0.01 | 0.38 |
| GO_RESPONSE_TO_EXTERNAL_STIMULUS | 117 | 44970 | 0.00 | 0.03 | 87 | 34412 | 0.02 | 0.21 | 30 | 10558 | 0.11 | 0.81 |
| GO_RESPONSE_TO_OXYGEN_CONTAINING_COMPOUND | 117 | 44639 | 0.00 | 0.03 | 80 | 31974 | 0.01 | 0.21 | 37 | 12664 | 0.16 | 0.92 |
| GO_RESPONSE_TO_LIPID | 74 | 28488 | 0.01 | 0.05 | 51 | 20165 | 0.07 | 0.31 | 23 | 8323.5 | 0.07 | 0.71 |
| GO_NEUTRAL_LIPID_METABOLIC_PROCESS | 26 | 10471 | 0.01 | 0.09 | 11 | 4670.9 | 0.06 | 0.30 | 15 | 5800.4 | 0.03 | 0.48 |
| GO_NEGATIVE_REGULATION_OF_HYDROLASE_ACTIVITY | 39 | 15809 | 0.00 | 0.03 | 29 | 11659 | 0.07 | 0.31 | 10 | 4150.4 | 0.01 | 0.38 |
| GO_ION_HOMEOSTASIS | 36 | 14581 | 0.00 | 0.03 | 28 | 11124 | 0.12 | 0.39 | 8 | 3456.5 | 0.01 | 0.34 |
| GO_RESPONSE_TO_BIOTIC_STIMULUS | 53 | 21055 | 0.00 | 0.03 | 42 | 17231 | 0.01 | 0.21 | 11 | 3824.1 | 0.26 | 1.00 |
| GO_EXOCYTOSIS | 29 | 12266 | 0.00 | 0.02 | 24 | 9996.6 | 0.02 | 0.21 | 5 | 2269.4 | 0.01 | 0.38 |
| GO_PLATELET_DEGRANULATION | 28 | 11805 | 0.00 | 0.02 | 23 | 9536.1 | 0.03 | 0.24 | 5 | 2269.4 | 0.01 | 0.38 |
| GO_RESPONSE_TO_ALCOHOL | 42 | 16602 | 0.01 | 0.06 | 30 | 12215 | 0.04 | 0.26 | 12 | 4387.7 | 0.13 | 0.85 |
| GO_REGULATED_EXOCYTOSIS | 29 | 12266 | 0.00 | 0.02 | 24 | 9996.6 | 0.02 | 0.21 | 5 | 2269.4 | 0.01 | 0.38 |
| GO_NEGATIVE_REGULATION_OF_PEPTIDASE_ACTIVITY | 30 | 12322 | 0.00 | 0.03 | 24 | 10014 | 0.02 | 0.21 | 6 | 2308.3 | 0.13 | 0.85 |
| GO_EXTRACELLULAR_MATRIX | 23 | 9693.5 | 0.00 | 0.03 | 15 | 6421.9 | 0.03 | 0.23 | 8 | 3271.7 | 0.03 | 0.48 |
| GO_SECRETORY_GRANULE | 31 | 12775 | 0.00 | 0.03 | 26 | 10505 | 0.07 | 0.31 | 5 | 2269.4 | 0.01 | 0.38 |
| GO_PLATELET_ALPHA_GRANULE | 21 | 8854.2 | 0.00 | 0.04 | 20 | 8393.7 | 0.03 | 0.23 | 1 | 460.52 | 0.38 | 1.00 |
| GO_SECRETORY_GRANULE_LUMEN | 25 | 10906 | 0.00 | 0.02 | 21 | 9096.7 | 0.00 | 0.18 | 4 | 1808.8 | 0.03 | 0.48 |
| GO_SECRETORY_VESICLE | 33 | 13409 | 0.00 | 0.04 | 27 | 10966 | 0.06 | 0.28 | 6 | 2443.2 | 0.06 | 0.68 |
| GO_VESICLE_LUMEN | 34 | 14920 | 0.00 | 0.02 | 29 | 12650 | 0.00 | 0.10 | 5 | 2269.4 | 0.01 | 0.38 |
| GO_BLOOD_MICROPARTICLE | 44 | 19027 | 0.00 | 0.02 | 36 | 15505 | 0.00 | 0.14 | 8 | 3521.8 | 0.00 | 0.24 |
| GO_INTRACELLULAR_VESICLE | 75 | 29546 | 0.00 | 0.03 | 57 | 22628 | 0.04 | 0.27 | 18 | 6917.8 | 0.02 | 0.44 |
| GO_EXTRACELLULAR_SPACE | 126 | 49402 | 0.00 | 0.02 | 90 | 36270 | 0.00 | 0.14 | 36 | 13132 | 0.02 | 0.48 |
| GO_CYTOPLASMIC_VESICLE_PART | 49 | 19540 | 0.00 | 0.03 | 38 | 15441 | 0.03 | 0.23 | 11 | 4098.9 | 0.11 | 0.80 |
| GO_PROTEIN_LIPID_COMPLEX | 20 | 8548.6 | 0.00 | 0.03 | 11 | 4585.6 | 0.10 | 0.37 | 9 | 3963 | 0.00 | 0.23 |
| GO_ENZYME_INHIBITOR_ACTIVITY | 35 | 14558 | 0.00 | 0.02 | 27 | 11014 | 0.05 | 0.27 | 8 | 3543.7 | 0.00 | 0.23 |
| GO_ENZYME_REGULATOR_ACTIVITY | 68 | 26821 | 0.00 | 0.03 | 49 | 19383 | 0.07 | 0.31 | 19 | 7437.9 | 0.01 | 0.38 |
| HALLMARK_HYPOXIA | 32 | 12844 | 0.01 | 0.10 | 29 | 12176 | 0.01 | 0.09 | 3 | 668.01 | 0.90 | 1.00 |
| HALLMARK_APOPTOSIS | 21 | 8486.6 | 0.02 | 0.10 | 14 | 5742.2 | 0.11 | 0.32 | 7 | 2744.4 | 0.08 | 0.53 |
| HALLMARK_COMPLEMENT | 22 | 8854.8 | 0.02 | 0.10 | 19 | 7868.5 | 0.05 | 0.30 | 3 | 986.27 | 0.50 | 0.99 |

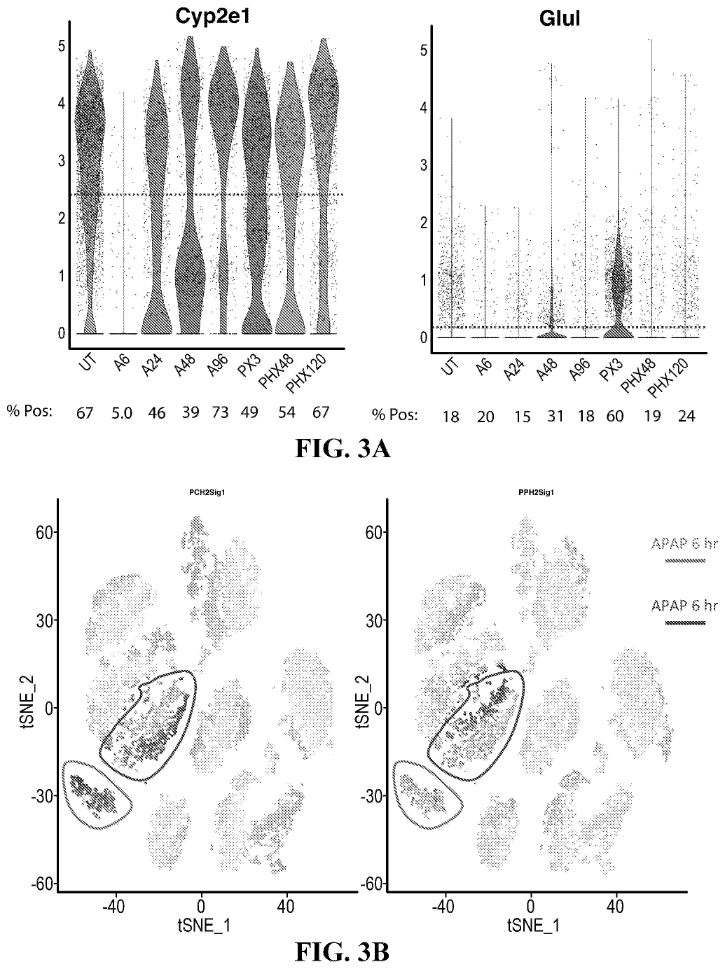

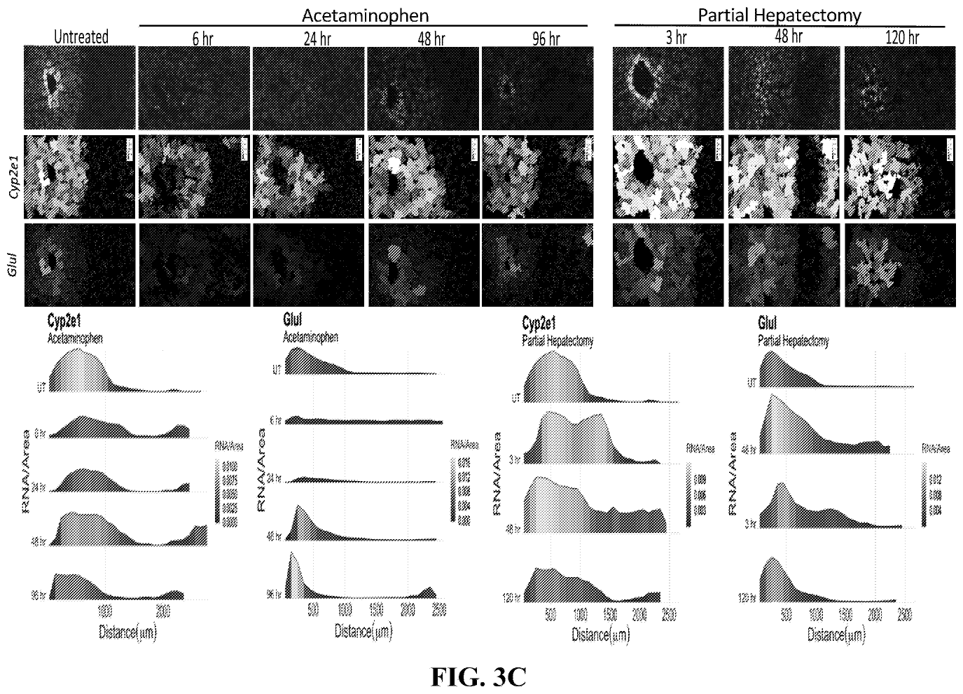

scRNA-seq is a powerful tool for the assessment of transcriptional changes following a perturbation, such as acute injury; however, the spatial location of each cell is lost following dissociation of the liver. Therefore, Applicants next wanted to validate, quantify, and spatially resolve genes of interest identified in the scRNA-Seq data set by performing smFISH analysis for select genes that span essential hepatic functions including: anti-oxidant response (Mt1, Txnrd1, Srxn1, Gclc), serum protein synthesis (Alb), glucose homeostasis (Pck1, Slc2a2), glutamate metabolism (Glut), and clotting factor synthesis (F2). Genes known to have either pericentral (Cyp2e1, Glu1, Sox9) or periportal (Lgr5, Tbx3, Axing, Arg1, Cdh1, Gls2, Ppargc1a) localization, as well as genes thought to be markers for hepatic stem cells (Sox9, Tbx3, Lgr5, Axing) are also targets. Applicants conducted a combinatorial analysis of smFISH by using an algorithm to define cellular outlines (CellProfiler) and counting transcripts (FISH-quant) to allow for a high-throughput method for analyzing the large number of genes that span many liver functions. FISH-quant was used to convert spot counts from every cell outline defined by CellProfiler into a representative heat map of the liver lobule for a given gene (e.g. Cyp2e1 and Glu1, FIG. 3C). smFISH analysis confirms the loss of the Cyp2e1-positive cell population directly surrounding the central vein in the APAP model at 6 and 24 hrs following exposure. Interestingly, the Cyp2e1-positive area spans an increased number of cells at 24 and 48 hrs post-APAP. This can be further confirmed by comparing the number of transcripts/area from a defined reference point (central vein) (FIG. 3C). Expression of Cyp2e1 reaches further into the midzone of the liver lobule following injury of the area directly surrounding the central vein in the APAP model. This phenomenon is further evident in the PH model in which the number of Cyp2e1-positive cells span further into the midzone of the liver lobule along with a dramatic increase in the number of Cyp2e1 transcripts/area. This suggests that midzonal and periportal hepatocytes have the ability to adapt to a loss of hepatocytes by upregulating genes that may not be normally expressed in that particular cell population (or zone) of the liver lobule.

Another example of functional plasticity is the expression of glutamate synthetase (Glut). As mentioned previously, Glu1 is normally expressed in a one cell thick layer of cells directly surrounding the central vein. As expected, this population of cells is eliminated following APAP exposure (6 and 24 hr post-APAP) (FIG. 3A). However, a small, but significant, amount of up-regulation of Glu1 can be observed in hepatocytes that span the entire liver lobule at both of these time points. This is further confirmed by the number of transcripts/area measured (FIG. 3C). The Glut-positive area returns to be primarily localized around the central vein by 48 hrs post-APAP. Interestingly, the PH model confirms the plasticity of hepatocytes in regard to Glu1 expression by revealing a marked up-regulation of the gene in pericentral hepatocytes, but also in midzonal hepatocytes, in which it is not normally expressed.

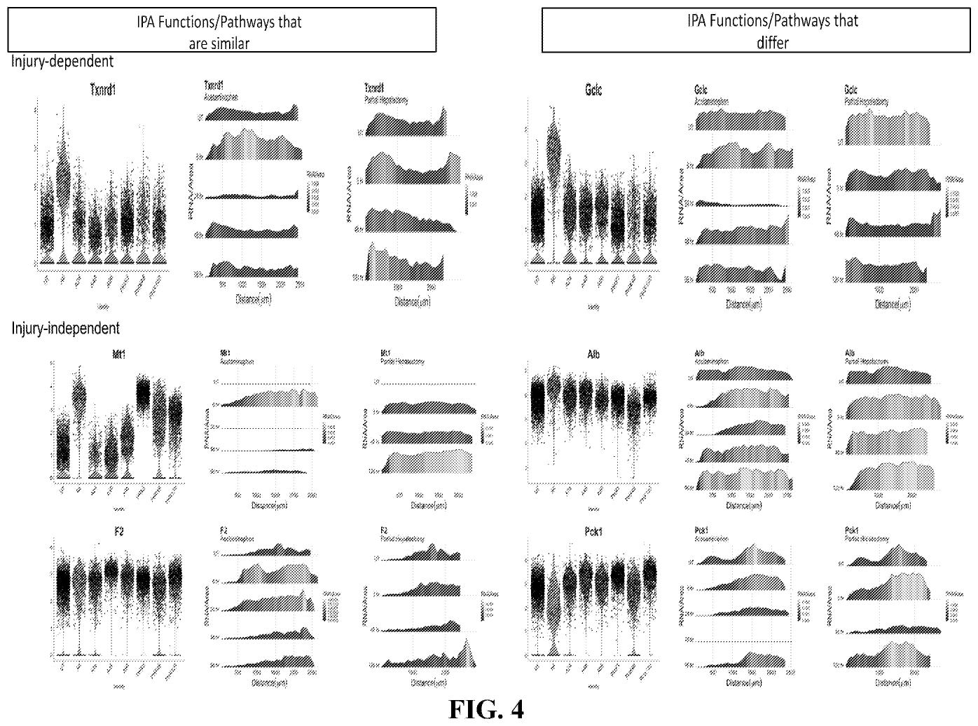

The status of functional hepatic marker genes was assessed in order to define the kinetics of transcriptional changes in an injury-dependent manner (FIG. 4). It is not surprising that select functions appear to be driven by injury mechanisms. For example, the anti-oxidant response appears to be strongest in the zone-dependent (APAP) model. This can be explained by the generation of ROS in this model, which drives the injury mechanism. Genes such as thioredoxin (Txnrd1) and sulfiredoxin (Srxn1) are classic anti-oxidant response genes that are most extremely up-regulated in the zone-dependent (APAP) model. However, there are an increased number of cells expressing these genes in the zone-independent (PH) model suggesting that there is still a compensatory expansion of expression due to the loss of functional tissue. It is also interesting to note that smFISH data reveals an up-regulation of these genes across the liver lobule reaching into the periportal region in the APAP model. This suggests that the compensatory up-regulation is not exclusive to the area surrounding injury, but that all hepatocytes can respond in a similar fashion.

A similar functional compensatory response can be seen for other classic hepatic marker genes (FIG. 4). Albumin, the most abundant serum protein, is produced by hepatocytes across the liver lobule with the highest expression in the periportal region. Acute injury results in a marked up-regulation of albumin across the entire liver lobule for both models. However, this up-regulation is most extreme in the PH model. Without being bound by theory, it is believed that this is due to differences in the amount of functional tissue loss between the two models. The PH model results in a much larger loss of functional tissue resulting in a higher demand for compensation. Similar observations can be made for genes such as the gluconeogenesis gene Pck1 and the coagulation factor F2.

One of the most dramatically changed genes following acute injury in both the zone-dependent (APAP) and -independent (PH) models is metallothionein (Mt1). Metallothionein has a high affinity for bivalent cations in the liver. It is thought to play an important role in the anti-oxidant response by providing a reservoir for bivalent cations in order to activate metalloenzymes, such as superoxide dismutase. Also, Mt1 has been shown to be induced following cellular damage and during the regenerative response where it is thought to provide cations for transcription factor and growth factor function. Applicants observe a massive increase in the expression of Mt1 in both zone-dependent (APAP) and -independent (PH) injury models within the earliest time points examined. The up-regulation of Mt1 happens in all hepatocytes highlighting not only the rapidity of hepatic functional adaptation, but also, the plasticity of the majority of hepatocytes across the liver lobule. MU expression returns to normal at 24 hrs post-APAP exposure, but can then be found in an increased number of hepatocytes at both 48 and 96 hrs post-APAP. This suggests that a second wave of Mt1 expression is important during the cell proliferation response. This is corroborated by the observation that Mt1 expression remains elevated throughout the PH time course, where an increased demand is present due to the increased loss of cell mass in this model.

### Hepatic Functional Compensation and Hepatocyte Proliferation

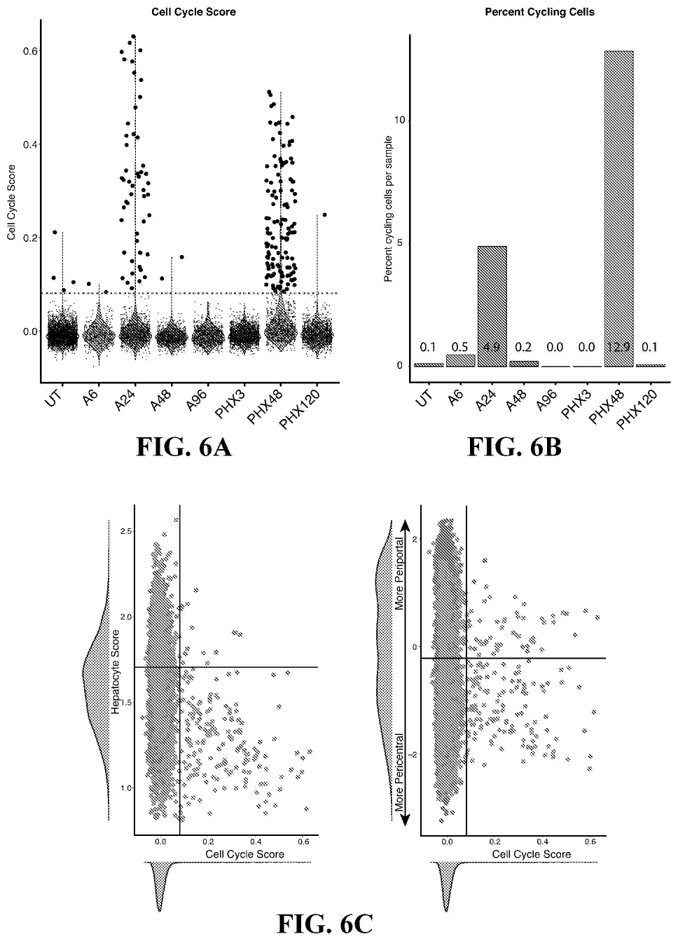

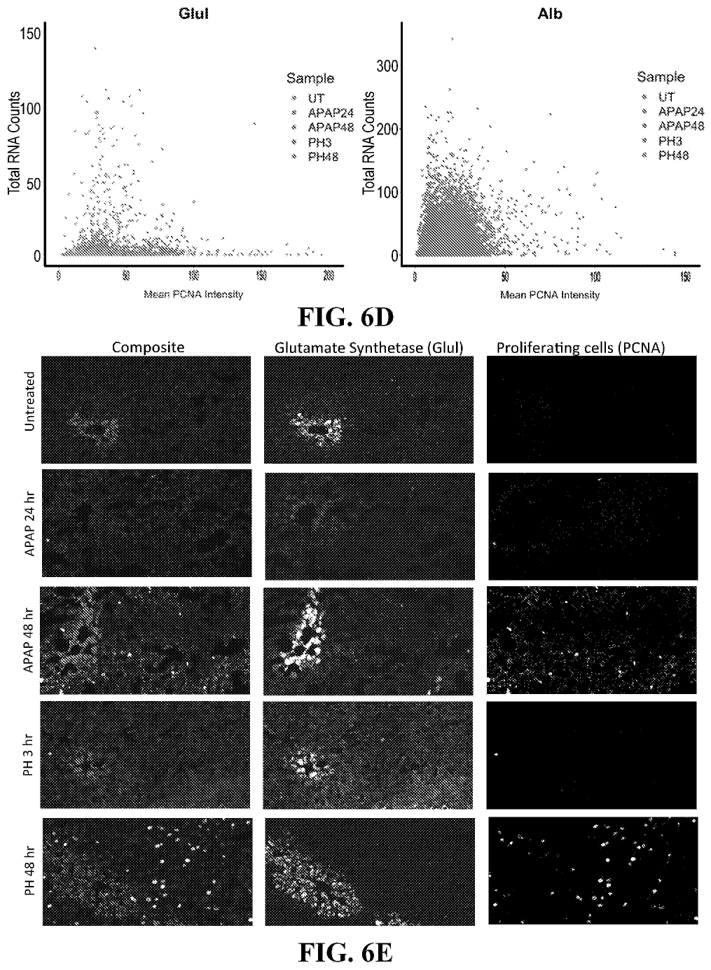

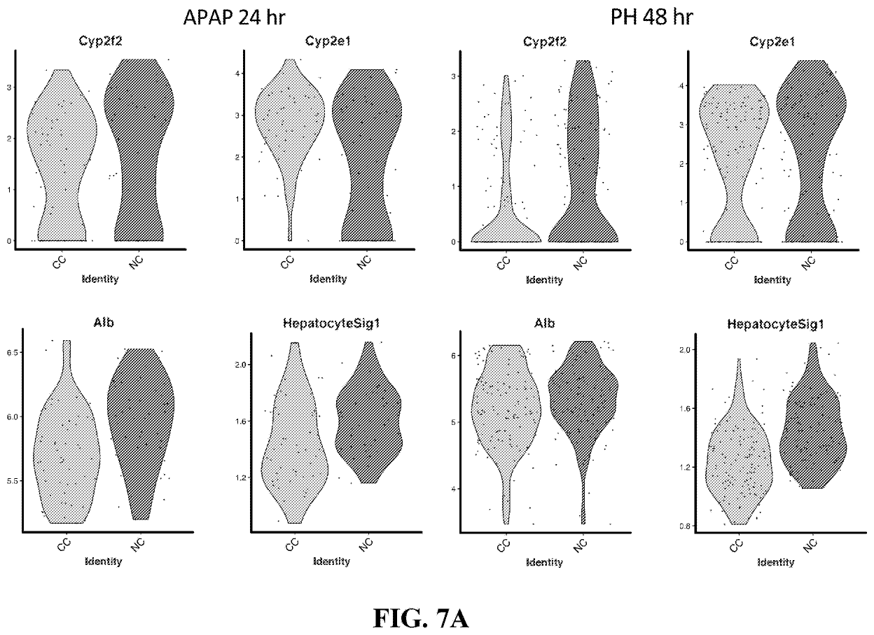

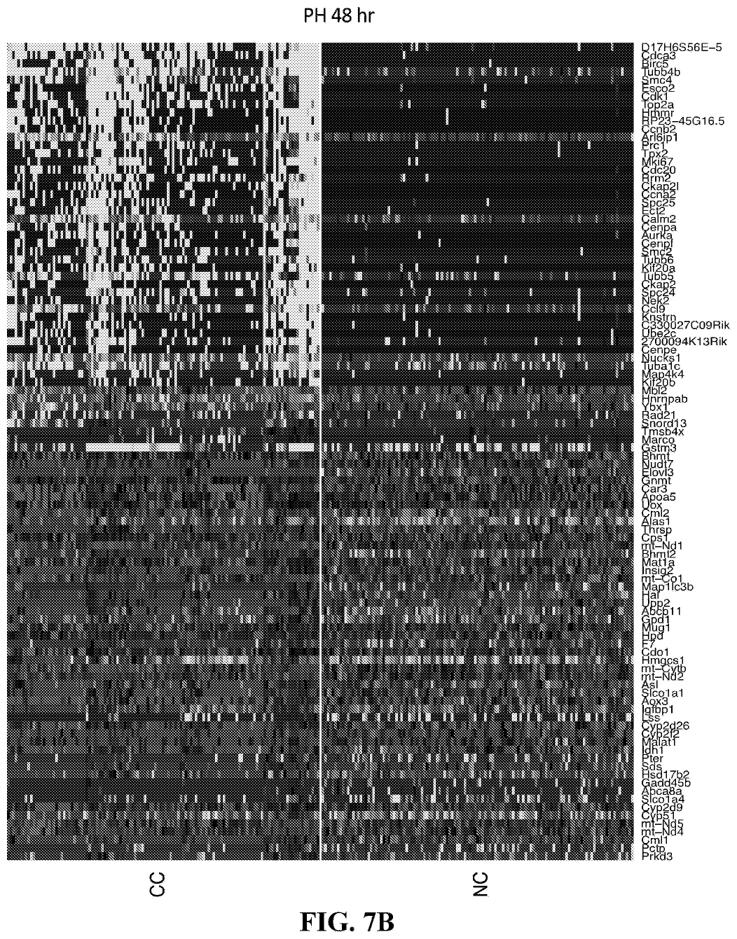

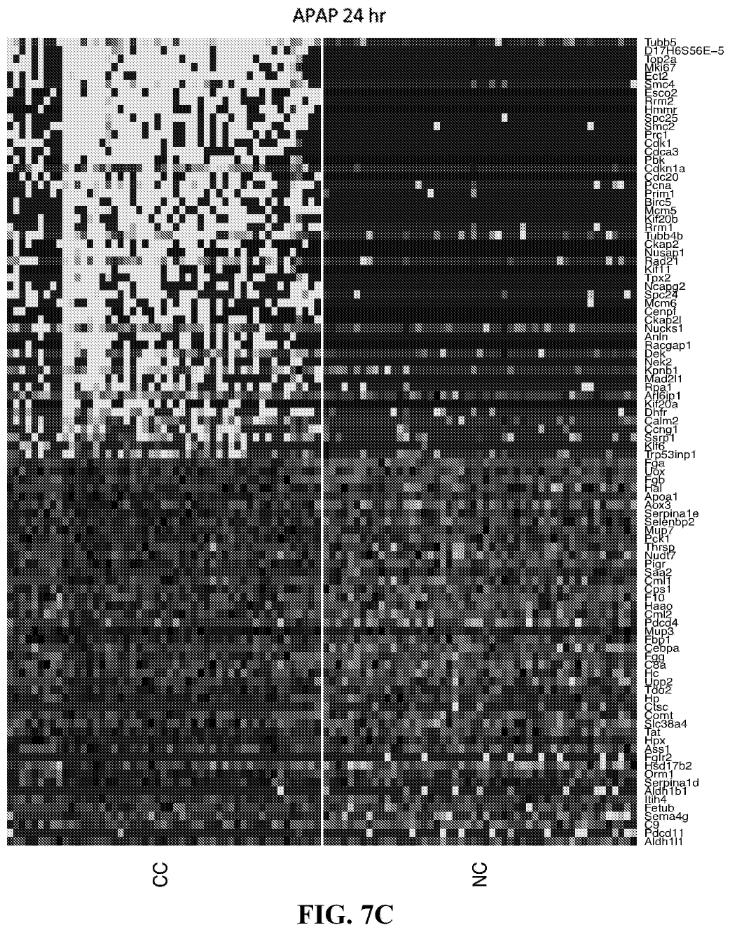

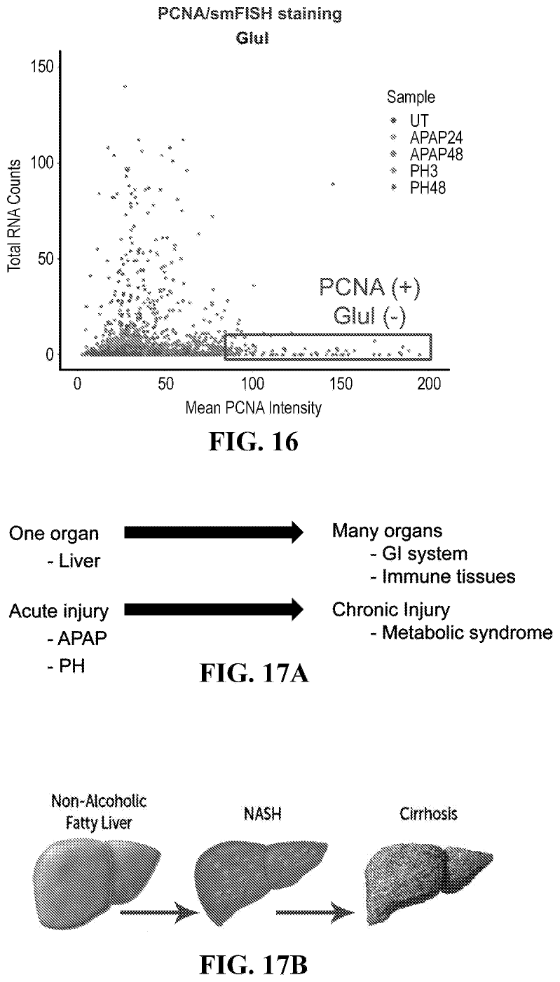

Liver function is known to be maintained with only slight alterations following acute liver injury. Here, Applicants describe a functional adaptive response throughout a time course of recovery. An important hallmark of liver regeneration is the ability for hepatocytes to proliferate following acute injury. This has been the most well-studied characteristic of liver regeneration to date. However, little is known about the maintenance of function within hepatocytes that are actively dividing. To investigate this, Applicants have performed a combinatorial analysis of gene expression (smFISH) and proliferation (PCNA immunofluorescence) in order to assess whether hepatocytes have the ability to both functionally adapt to a loss of tissue while maintaining the ability to proliferate or if these are mutually exclusive events. (See, e.g. FIGS. 6D, 6E, 16)

### Mechanisms Surrounding the Maintenance of Liver Function: A Novel Dual Role for Wnt Signaling

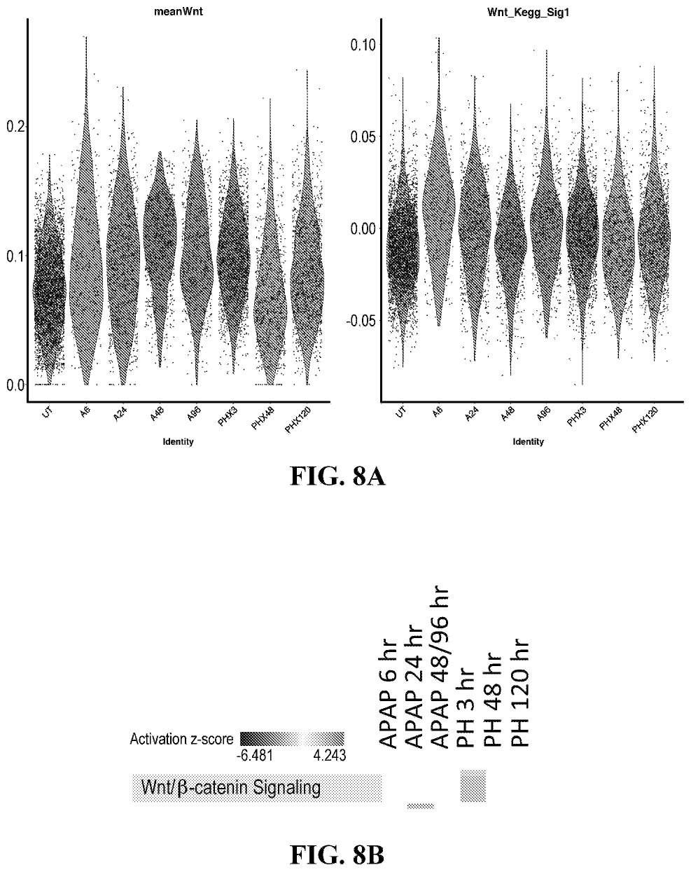

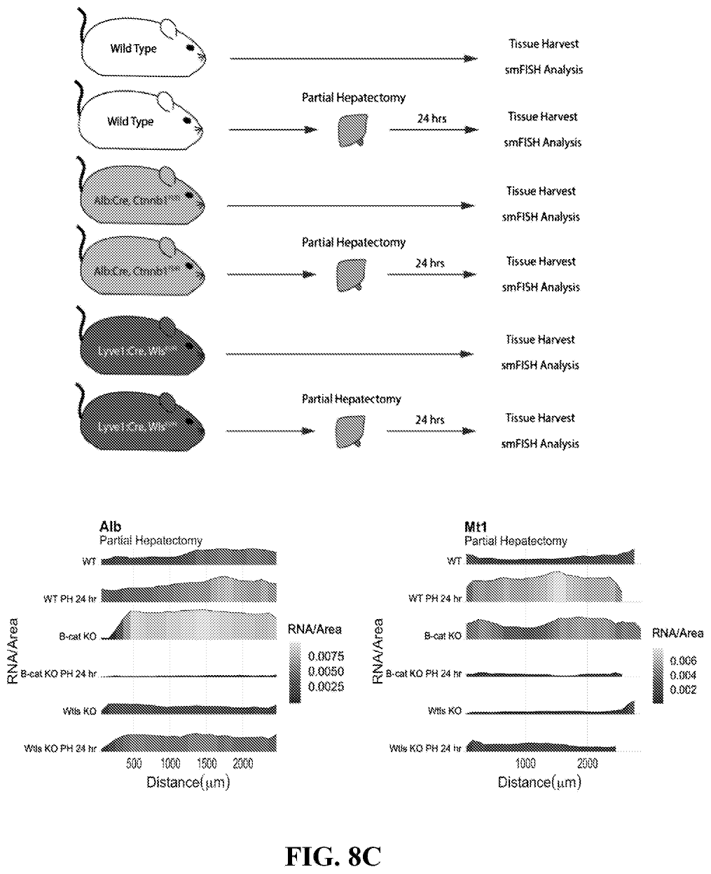

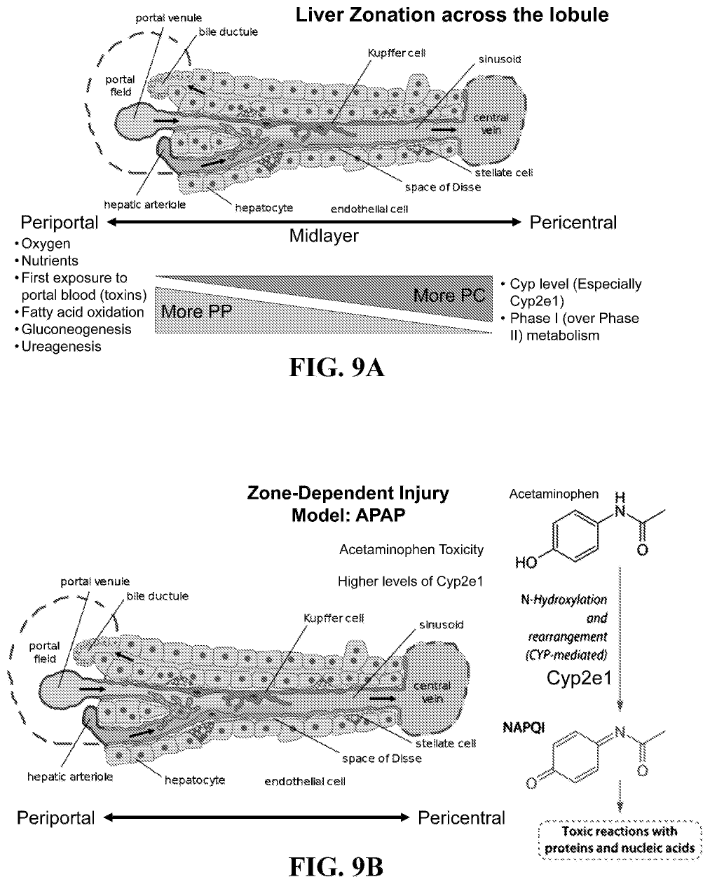

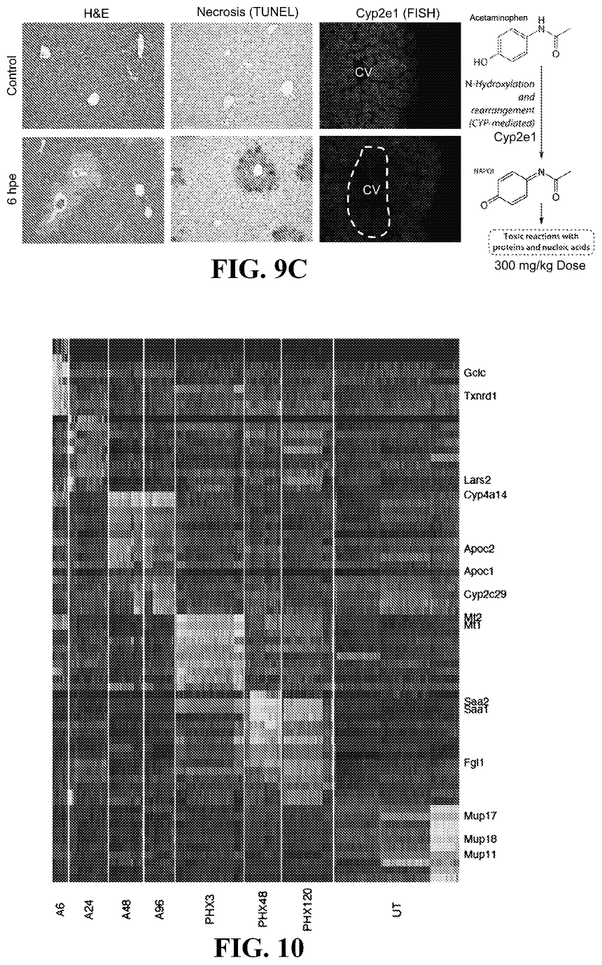

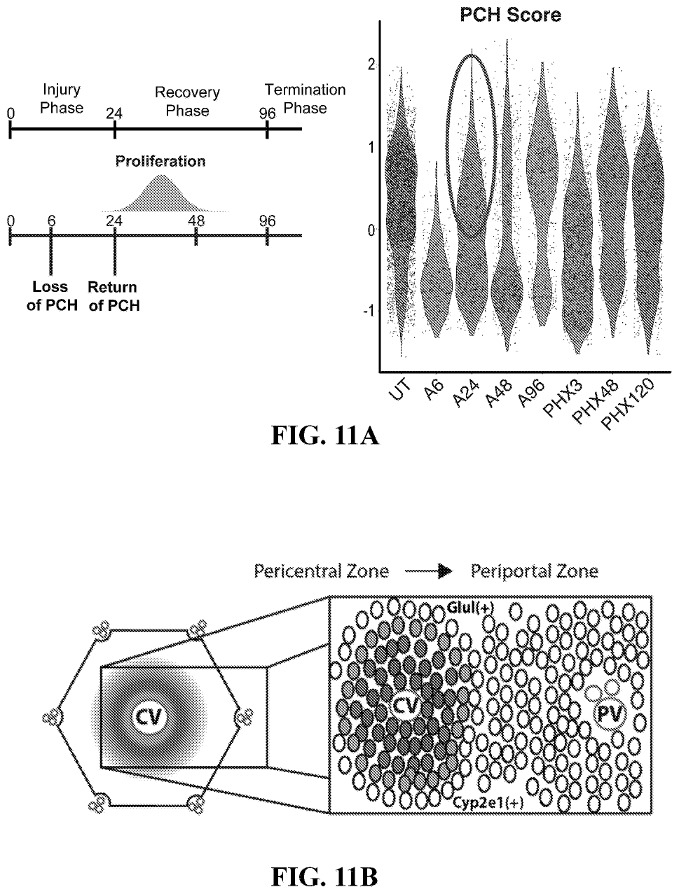

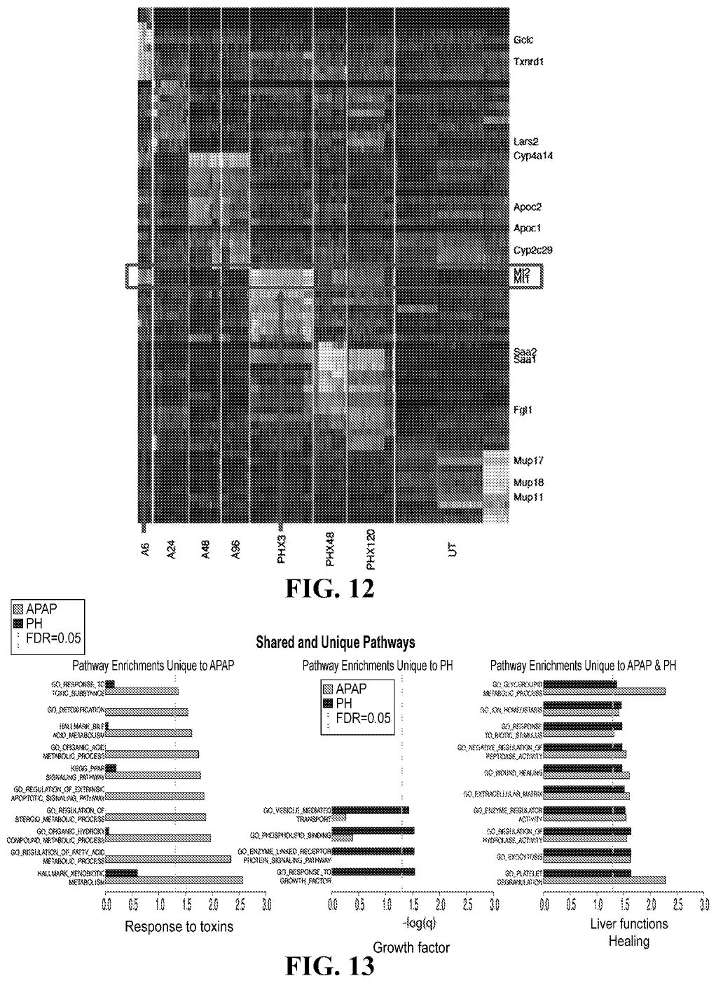

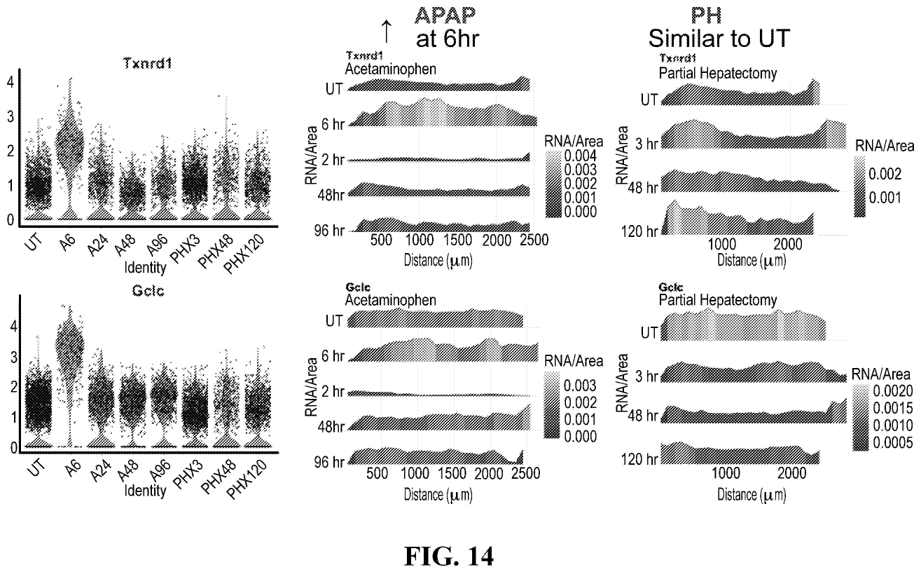

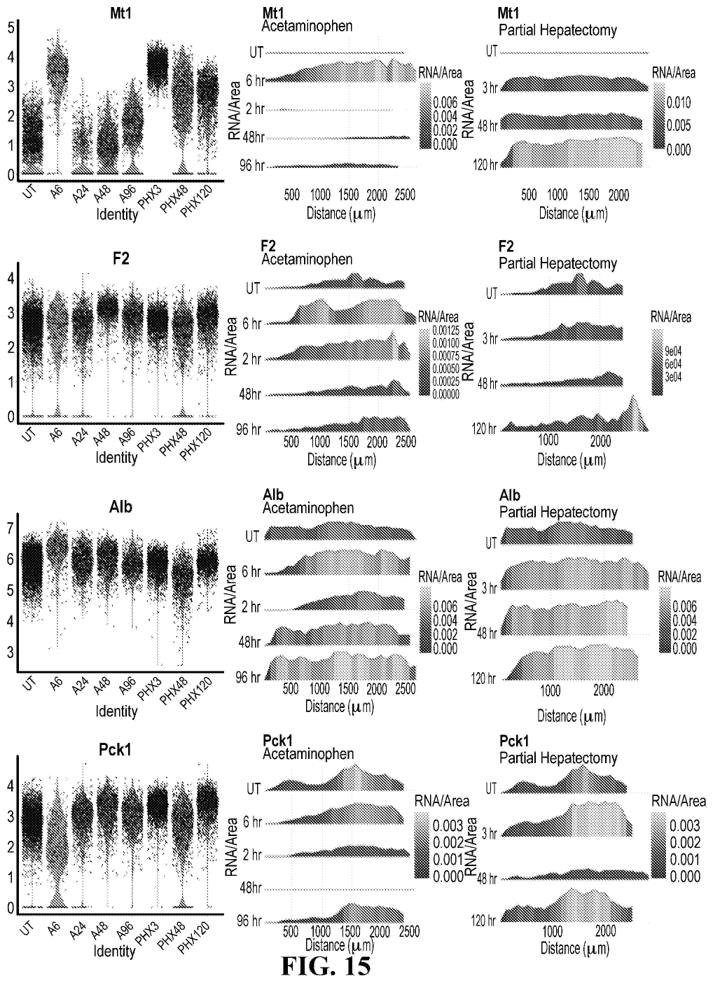

The next aim was to identify potential signaling cascades that respond to acute liver injury to promote both functional adaptation through transcriptional changes and promotion of cell proliferation to replenish lost cell mass. The Wnt signaling pathway fits both categories as it is known to play an important role in both the establishment of hepatic zonation as well as being necessary for the cell proliferation response during hepatic regeneration. This suggests that Wnt signaling may play a dual role in the liver following acute injury by which it not only promotes cell proliferation and a return to pre-injury cell number and mass but that the pathway also activates reprogramming of already present hepatocytes to maintain essential hepatic function. (FIG. 8A-8C)

The study in this example provides a novel view of liver regeneration revealing a functional compensatory response to lost functional mass during acute liver injury.

### Methods

Liver dissociation: At time period for evaluation post-injury, liver was dissociated by the following steps: Anesthetize mouse, Open abdomen, Clamp the thoracic inferior vena cava, Insert catheter into abdominal inferior vena cava, Perfuse liver with perfusion medium and cut portal vein for drainage, Perfuse liver with digestion medium, Remove liver.

Study of gene expression patterns across liver lobule followed the general approach of K B Halpern et al. Nature 1-5 (2017) doi:10.1038/nature21065, defining a spatial barcode of zonated landmark genes in untreated mice. This approach was utilized as a starting point for the study of gene expression patterns in the PH and APAP injured livers.

### Example 2—High Fat Diet as Model for Chronic Injury

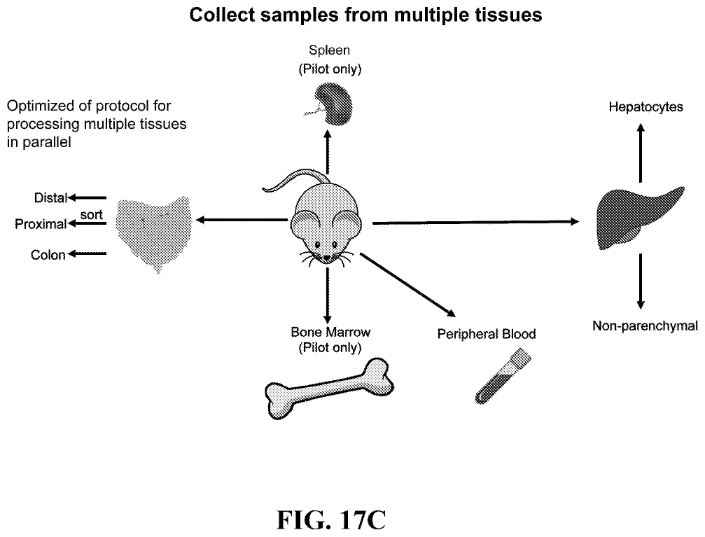

As provided in FIG. 17A, future studies will be used to extend the investigation described in Example 1 to biologically expand analysis to other organs and from acute injury to chronic injury. High fat diets can lead to liver and intestinal inflammation and cancer. Use of High Fat Diet mouse models will extend the approach from profiling alone to multiple GI and immune issues in a chronic injury model, allowing identification and characterization of potential cancer progenitors in the gut and liver. Utilization of this model will allow exploration of cross-talk between GI organs through immune cells, hormones, or other molecules (e.g. bile acids. A schematic of a protocol that can be used in biological expansion studies utilizing high fat diet will include study of multiple organs including liver and intestines with subsequent sorting of cells, peripheral blood, and spleen and bone marrow (pilot only) (FIG. 17C). Distinct cell types and shift in expression between control diet and high fat diet subjects can be identified, including clustering of intestinal, liver and immune cell types and shifts by diets in particular cell types using approaches disclosed herein.

Further studies can identify reproducible shifts in cell types or expression patterns between diet conditions. Hepatic organoid assays will be performed to assess organoid forming efficiency and effect of lipids on organoid growth. Use of smFISH or IHC imaging to further characterize genes/proteins and pathways of interest in space would be a further goal of the extended biological studies.

### Example 3—Acute Injury

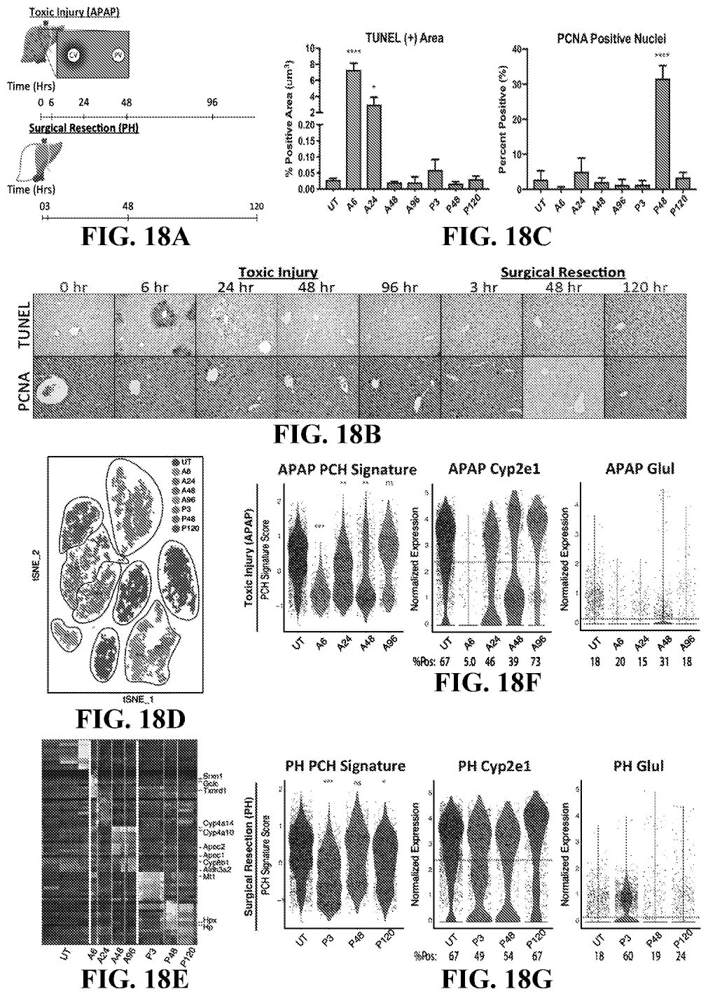

Applicants investigated compensatory responses in the liver during key phases of liver regeneration in both a toxic (APAP) model and following surgical resection (partial hepatectomy, PH) (FIG. 18A). Utilizing the powerful combinatorial approach of Seq-Well, a massively-parallel single-cell RNA-seq (scRNA-Seq) platform ideally suited for fragile cells like hepatocytes, and single-molecule fluorescent in situ hybridization (smFISH), Applicants define transcriptional changes after injury⁹. Disclosed herein is the discovery that remaining hepatocytes functionally compensate for lost liver mass by increased transcriptional output of key hepatocyte genes. Importantly, hepatocytes also alter their zone-dictated functional identities within the liver lobule to help maintain global expression of select transcripts. These studies find that hepatocyte functional compensation precedes the peak phase of cell proliferation and that cycling cells do not participate to the same degree as non-cycling hepatocytes during the regeneration phase. Both cycling and non-cycling cells show upregulation of targets of Wnt signaling—known to play a central role in normal hepatocyte development, maintenance and liver regeneration. Applicants demonstrate that compensation depends on intact β-catenin activation through macrophage-secreted Wnts. Overall, our results identify previously unappreciated plasticity among hepatocytes during a newly discovered compensatory phase after liver injury, as well as Wnt/β-catenin signaling as a therapeutically relevant pathway for maintaining and re-establishing homeostatic liver function.

Transcriptional Adaption after Liver Injury

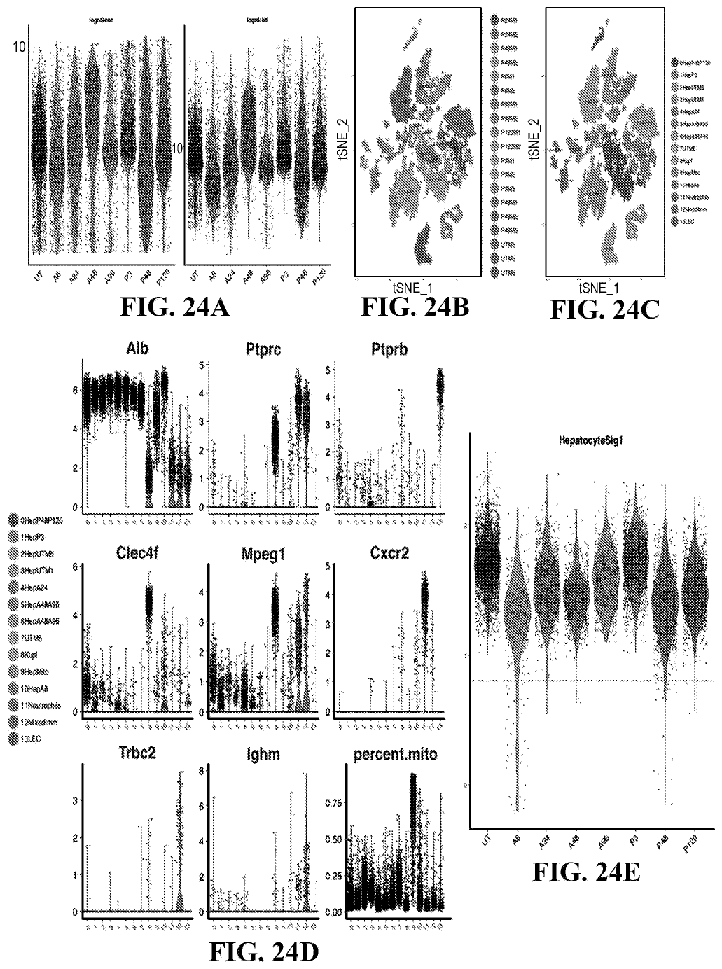

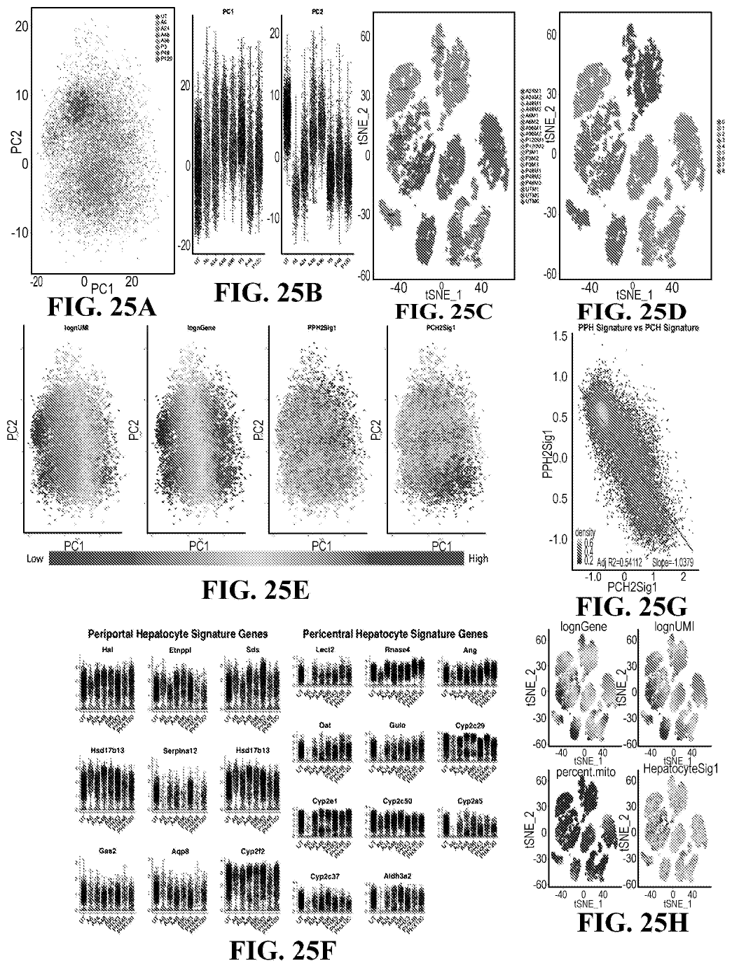

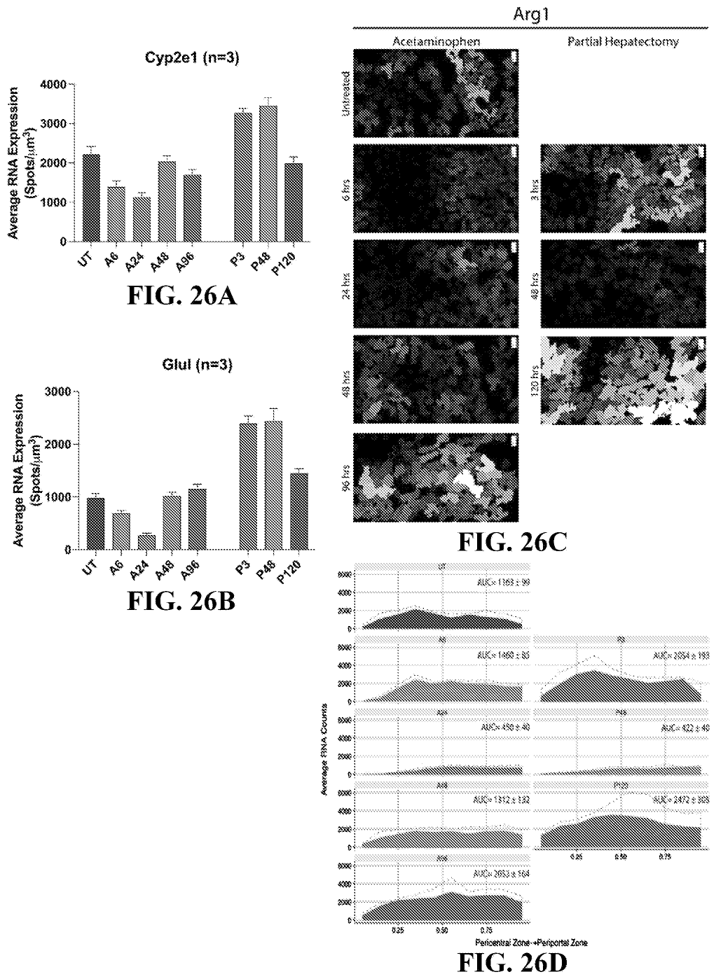

To assess global transcriptional shifts in hepatocytes at single-cell resolution following acute liver injury, Applicants employed scRNA-Seq to characterize response dynamics in both PH and APAP models, capturing the injury, regeneration, and termination phases of liver regeneration⁴ (FIG. 18B, 18C). Applicants profiled a total of 16,019 cells across 19 different experiments to an average sequencing depth of >48,000 reads/cell (FIG. 24A-24C, Methods). Immune and endothelial cell types as well as low quality cells were filtered out from the dataset, retaining 10,762 high-quality hepatocyte transcriptomes for subsequent analyses (FIG. 24D, 24E, Table 12, Methods). Shared nearest neighbour clustering (SNN) visualized on a t-Stochastic Neighbor Embedding (t-SNE) plot revealed hepatocyte populations that cluster by injury model and post-injury time point (FIG. 18D, Methods).

TABLE 12
Sequencing metrics table containing Average reads, Average genes,
Average UMIs, and total number of cells passing quality and hepatocyte
identitiy filtering for each sample in the dataset.

| | Ave | Ave | Ave | |
|---|---|---|---|---|
| Sample | ReadsAll | GenesAll | UMIsAll | nCellsFil |
| UTM1 | 29350 | 2345 | 11709 | 1188 |
| UTM5 | 31794 | 1705 | 8312 | 1435 |
| UTM6 | 79301 | 892 | 4003 | 778 |
| A6M1 | 34132 | 1298 | 6010 | 291 |
| A6M2 | 27584 | 1315 | 5696 | 128 |
| A24M1 | 34357 | 1948 | 9366 | 852 |
| A24M2 | 18777 | 828 | 3997 | 191 |
| A48M1 | 28188 | 2490 | 14543 | 790 |
| A48M2 | 54952 | 1536 | 14358 | 120 |
| A96M1 | 35995 | 2165 | 11572 | 757 |
| A96M2 | 441 | 126 | 441 | 52 |
| PHX3M1 | 99976 | 3320 | 35423 | 249 |
| PHX3M2 | 20333 | 2149 | 10102 | 1330 |
| PHX3M3 | 92468 | 3222 | 32670 | 266 |
| PHX48M1 | 11125 | 1139 | 5850 | 165 |
| PHX48M2 | 54248 | 1976 | 12051 | 440 |
| PHX48M3 | 92468 | 1324 | 6648 | 352 |
| PHX120M1 | 93284 | 3216 | 26576 | 287 |
| PHX120M2 | 22696 | 1826 | 8031 | 1091 |

TABLE 13
Differentially expressed genes (DEG) between the three untreated (UT) samples. Calculated
using the FindAllMarkers function in the R package Seurat using the Wilcox test.

| | p_val | avg_logFC | pct.1 | pct.2 | p_val_adj | cluster | gene |
|---|---|---|---|---|---|---|---|
| Thrsp | 0 | 1.21818366 | 0.977 | 0.439 | 0 | UTM1 | Thrsp |
| Nudt7 | 0 | 1.11928024 | 0.998 | 0.74 | 0 | UTM1 | Nudt7 |
| Scd1 | 0 | 0.99322463 | 1 | 0.848 | 0 | UTM1 | Scd1 |
| Hsd3b5 | 0 | 0.97409205 | 0.944 | 0.396 | 0 | UTM1 | Hsd3b5 |
| Cfhr2 | 0 | 0.81994799 | 0.998 | 0.798 | 0 | UTM1 | Cfhr2 |
| Uox | 0 | 0.71024072 | 0.999 | 0.947 | 0 | UTM1 | Uox |
| Trf | 0 | 0.52918211 | 1 | 1 | 0 | UTM1 | Trf |
| Aadac | 7.97E−305 | 0.81646299 | 0.999 | 0.786 | 1.98E−300 | UTM1 | Aadac |
| Gnmt | 4.34E−304 | 0.52785298 | 1 | 0.982 | 1.08E−299 | UTM1 | Gnmt |
| Inmt | 1.17E−288 | 0.82783916 | 0.998 | 0.822 | 2.90E−284 | UTM1 | Inmt |
| Alb | 3.66E−274 | 0.48743601 | 1 | 1 | 9.08E−270 | UTM1 | Alb |
| Car3 | 1.52E−269 | 0.59669431 | 1 | 0.96 | 3.78E−265 | UTM1 | Car3 |
| Keg1 | 6.58E−250 | 0.78906431 | 0.896 | 0.357 | 1.63E−245 | UTM1 | Keg1 |
| Acly | 4.57E−246 | 0.76348807 | 0.815 | 0.246 | 1.14E−241 | UTM1 | Acly |
| Sc5d | 3.32E−228 | 0.7161617 | 0.984 | 0.665 | 8.24E−224 | UTM1 | Sc5d |
| Cyp7a1 | 2.92E−219 | 0.61454812 | 0.59 | 0.081 | 7.25E−215 | UTM1 | Cyp7a1 |
| Otc | 1.35E−217 | 0.55985355 | 0.998 | 0.869 | 3.34E−213 | UTM1 | Otc |
| Akr1c6 | 5.76E−213 | 0.53304224 | 1 | 0.955 | 1.43E−208 | UTM1 | Akr1c6 |
| G6pc | 3.04E−212 | 0.68559678 | 0.976 | 0.631 | 7.55E−208 | UTM1 | G6pc |
| Cyp2c70 | 4.92E−202 | 0.63146884 | 0.988 | 0.751 | 1.22E−197 | UTM1 | Cyp2c70 |
| Atp5f1 | 2.34E−201 | 0.57886961 | 0.995 | 0.801 | 5.80E−197 | UTM1 | Atp5f1 |
| Fbp1 | 6.59E−199 | 0.51619137 | 0.998 | 0.906 | 1.64E−194 | UTM1 | Fbp1 |
| Fabp1 | 5.43E−197 | 0.309557 | 1 | 1 | 1.35E−192 | UTM1 | Fabp1 |
| Hsd11b1 | 2.73E−196 | 0.56232512 | 0.994 | 0.811 | 6.77E−192 | UTM1 | Hsd11b1 |
| Adipor2 | 7.77E−196 | 0.63114559 | 0.98 | 0.714 | 1.93E−191 | UTM1 | Adipor2 |
| Serpina3c | 1.06E−195 | 0.50459729 | 0.755 | 0.186 | 2.64E−191 | UTM1 | Serpina3c |
| Sepp1 | 2.79E−195 | 0.28399703 | 1 | 1 | 6.92E−191 | UTM1 | Sepp1 |
| Aacs | 5.61E−195 | 0.40975071 | 0.441 | 0.032 | 1.39E−190 | UTM1 | Aacs |
| Mgst1 | 1.31E−193 | 0.47663366 | 1 | 0.996 | 3.24E−189 | UTM1 | Mgst1 |
| Fasn | 2.04E−186 | 0.55115081 | 0.675 | 0.168 | 5.06E−182 | UTM1 | Fasn |
| Egfr | 1.52E−185 | 0.64603405 | 0.981 | 0.716 | 3.79E−181 | UTM1 | Egfr |
| Hhex | 1.16E−184 | 0.49641357 | 0.63 | 0.134 | 2.87E−180 | UTM1 | Hhex |
| Fh1 | 1.19E−179 | 0.62319183 | 0.929 | 0.498 | 2.96E−175 | UTM1 | Fh1 |
| Cat | 7.06E−179 | 0.43531632 | 0.999 | 0.955 | 1.75E−174 | UTM1 | Cat |
| Errfi1 | 2.37E−173 | 0.51395691 | 0.997 | 0.864 | 5.88E−169 | UTM1 | Errfi1 |
| Ddc | 1.65E−172 | 0.56196541 | 0.791 | 0.282 | 4.10E−168 | UTM1 | Ddc |
| Elovl2 | 1.71E−170 | 0.58928921 | 0.947 | 0.566 | 4.24E−166 | UTM1 | Elovl2 |
| Prdx4 | 9.64E−163 | 0.6055411 | 0.884 | 0.458 | 2.39E−158 | UTM1 | Prdx4 |
| Cth | 6.01E−162 | 0.54591986 | 0.991 | 0.756 | 1.49E−157 | UTM1 | Cth |
| Cps1 | 8.04E−162 | 0.43042778 | 1 | 0.956 | 2.00E−157 | UTM1 | Cps1 |
| Igf1 | 3.51E−161 | 0.47768807 | 0.994 | 0.855 | 8.71E−157 | UTM1 | Igf1 |
| Bhmt | 6.12E−161 | 0.43322273 | 0.999 | 0.934 | 1.52E−156 | UTM1 | Bhmt |
| Mup3 | 1.14E−159 | 0.34482089 | 1 | 1 | 2.84E−155 | UTM1 | Mup3 |
| Elovl5 | 8.41E−159 | 0.61224151 | 0.868 | 0.425 | 2.09E−154 | UTM1 | Elovl5 |
| Cyp2f2 | 1.40E−158 | 0.60967373 | 0.982 | 0.799 | 3.48E−154 | UTM1 | Cyp2f2 |
| Fgb | 2.78E−158 | 0.28109091 | 1 | 1 | 6.90E−154 | UTM1 | Fgb |
| Serpind1 | 2.39E−156 | 0.53913725 | 0.974 | 0.676 | 5.94E−152 | UTM1 | Serpind1 |
| Echdc2 | 8.02E−154 | 0.52918421 | 0.796 | 0.315 | 1.99E−149 | UTM1 | Echdc2 |
| Hspa5 | 2.56E−153 | 0.43540717 | 0.999 | 0.914 | 6.36E−149 | UTM1 | Hspa5 |
| Slc10a1 | 1.72E−152 | 0.51702656 | 0.987 | 0.765 | 4.27E−148 | UTM1 | Slc10a1 |
| Serp1 | 1.63E−151 | 0.55530728 | 0.944 | 0.601 | 4.05E−147 | UTM1 | Serp1 |
| Aqp8 | 2.56E−150 | 0.5507968 | 0.817 | 0.338 | 6.36E−146 | UTM1 | Aqp8 |
| Slco1a1 | 4.64E−150 | 0.52689959 | 0.97 | 0.674 | 1.15E−145 | UTM1 | Slco1a1 |
| C9 | 8.51E−150 | 0.43561512 | 0.995 | 0.908 | 2.11E−145 | UTM1 | C9 |
| Cstf3 | 1.59E−143 | 0.3219351 | 0.423 | 0.058 | 3.95E−139 | UTM1 | Cstf3 |
| Mdh1 | 6.57E−143 | 0.46155291 | 0.988 | 0.832 | 1.63E−138 | UTM1 | Mdh1 |
| Hmgcr | 1.22E−142 | 0.42082632 | 0.545 | 0.126 | 3.04E−138 | UTM1 | Hmgcr |
| Hsd17b6 | 1.38E−142 | 0.52097308 | 0.769 | 0.284 | 3.42E−138 | UTM1 | Hsd17b6 |
| Qdpr | 2.12E−141 | 0.50438731 | 0.982 | 0.723 | 5.28E−137 | UTM1 | Qdpr |
| Hrsp12 | 1.29E−140 | 0.44167003 | 0.995 | 0.908 | 3.20E−136 | UTM1 | Hrsp12 |
| Serpinc1 | 1.43E−140 | 0.32566392 | 1 | 0.974 | 3.54E−136 | UTM1 | Serpinc1 |
| Hes6 | 7.04E−140 | 0.46539249 | 0.722 | 0.258 | 1.75E−135 | UTM1 | Hes6 |
| 1500017E21Rik | 4.96E−139 | 0.54206772 | 0.774 | 0.317 | 1.23E−134 | UTM1 | 1500017E21Rik |
| Gm4788 | 8.33E−139 | 0.49918037 | 0.965 | 0.659 | 2.07E−134 | UTM1 | Gm4788 |
| Tubb2a | 1.08E−138 | 0.473813 | 0.735 | 0.268 | 2.68E−134 | UTM1 | Tubb2a |
| Deb1 | 3.87E−135 | 0.49455146 | 0.737 | 0.285 | 9.61E−131 | UTM1 | Deb1 |
| Ttc39c | 8.05E−135 | 0.5016707 | 0.907 | 0.501 | 2.00E−130 | UTM1 | Ttc39c |
| Ndufv2 | 1.68E−134 | 0.5030504 | 0.905 | 0.51 | 4.18E−130 | UTM1 | Ndufv2 |
| Ndufc2 | 1.17E−133 | 0.54556827 | 0.86 | 0.428 | 2.90E−129 | UTM1 | Ndufc2 |
| Aldob | 2.10E−133 | 0.27645126 | 1 | 0.995 | 5.22E−129 | UTM1 | Aldob |
| Gls2 | 1.90E−130 | 0.54901012 | 0.91 | 0.522 | 4.71E−126 | UTM1 | Gls2 |
| Arg1 | 1.92E−129 | 0.33011144 | 1 | 0.972 | 4.76E−125 | UTM1 | Arg1 |
| Ass1 | 2.99E−127 | 0.37456042 | 0.998 | 0.942 | 7.42E−123 | UTM1 | Ass1 |
| Hmgcs1 | 5.26E−125 | 0.52691875 | 0.932 | 0.581 | 1.31E−120 | UTM1 | Hmgcs1 |
| B2m | 5.63E−124 | 0.35038582 | 0.999 | 0.944 | 1.40E−119 | UTM1 | B2m |
| Selk | 5.20E−122 | 0.4300711 | 0.773 | 0.332 | 1.29E−117 | UTM1 | Selk |
| Cfhr1 | 3.40E−121 | 0.48091782 | 0.838 | 0.43 | 8.44E−117 | UTM1 | Cfhr1 |
| Insig1 | 3.75E−121 | 0.42772787 | 0.713 | 0.272 | 9.31E−117 | UTM1 | Insig1 |
| Ndufs4 | 9.96E−120 | 0.4886043 | 0.831 | 0.418 | 2.47E−115 | UTM1 | Ndufs4 |
| Sqle | 1.16E−119 | 0.40342481 | 0.623 | 0.202 | 2.88E−115 | UTM1 | Sqle |
| Tat | 7.79E−119 | 0.41673949 | 0.994 | 0.854 | 1.93E−114 | UTM1 | Tat |
| Vmp1 | 1.20E−118 | 0.44115404 | 0.765 | 0.329 | 2.99E−114 | UTM1 | Vmp1 |
| Aox3 | 1.82E−117 | 0.43544215 | 0.99 | 0.843 | 4.52E−113 | UTM1 | Aox3 |
| Adh6-ps1 | 5.83E−117 | 0.37703004 | 0.577 | 0.172 | 1.45E−112 | UTM1 | Adh6-ps1 |
| C1d | 9.18E−117 | 0.38281513 | 0.626 | 0.208 | 2.28E−112 | UTM1 | C1d |
| Slc22a30 | 1.04E−116 | 0.40119661 | 0.779 | 0.338 | 2.58E−112 | UTM1 | Slc22a30 |
| Tmem30a | 8.89E−116 | 0.46673515 | 0.899 | 0.538 | 2.21E−111 | UTM1 | Tmem30a |
| D4Wsu53e | 2.39E−115 | 0.38697768 | 0.671 | 0.232 | 5.93E−111 | UTM1 | D4Wsu53e |
| Hacl1 | 1.19E−113 | 0.43962629 | 0.971 | 0.703 | 2.95E−109 | UTM1 | Hacl1 |
| Cml1 | 1.24E−113 | 0.46331845 | 0.949 | 0.654 | 3.08E−109 | UTM1 | Cml1 |
| Cmtm6 | 1.39E−113 | 0.36019667 | 0.614 | 0.204 | 3.45E−109 | UTM1 | Cmtm6 |
| H2-K1 | 1.60E−112 | 0.36327199 | 1 | 0.902 | 3.97E−108 | UTM1 | H2-K1 |
| 2810007J24Rik | 5.68E−112 | 0.36295773 | 0.997 | 0.913 | 1.41E−107 | UTM1 | 2810007J24Rik |
| Cxadr | 1.23E−111 | 0.38792689 | 0.607 | 0.204 | 3.05E−107 | UTM1 | Cxadr |
| Stard4 | 2.16E−111 | 0.3943334 | 0.67 | 0.253 | 5.35E−107 | UTM1 | Stard4 |
| Slc26a1 | 1.22E−108 | 0.42555394 | 0.776 | 0.343 | 3.02E−104 | UTM1 | Slc26a1 |
| Ppib | 1.80E−106 | 0.42232707 | 0.955 | 0.666 | 4.47E−102 | UTM1 | Ppib |
| Hao1 | 4.36E−105 | 0.41128035 | 0.944 | 0.658 | 1.08E−100 | UTM1 | Hao1 |
| Nit2 | 5.05E−105 | 0.41499963 | 0.836 | 0.437 | 1.25E−100 | UTM1 | Nit2 |
| Gas2 | 9.53E−105 | 0.40152064 | 0.614 | 0.221 | 2.37E−100 | UTM1 | Gas2 |
| Eny2 | 3.13E−104 | 0.3871366 | 0.706 | 0.292 | 7.77E−100 | UTM1 | Eny2 |
| Lifr | 1.83E−103 | 0.4005345 | 0.91 | 0.578 | 4.54E−99 | UTM1 | Lifr |
| mt-Cytb | 3.21E−103 | 0.32028033 | 1 | 0.999 | 7.97E−99 | UTM1 | mt-Cytb |
| Dak | 1.12E−102 | 0.40580329 | 0.828 | 0.421 | 2.78E−98 | UTM1 | Dak |
| Elovl6 | 4.00E−102 | 0.29994929 | 0.465 | 0.12 | 9.93E−98 | UTM1 | Elovl6 |
| Nme1 | 9.03E−102 | 0.41494271 | 0.874 | 0.486 | 2.24E−97 | UTM1 | Nme1 |
| Pklr | 1.39E−101 | 0.33117616 | 0.631 | 0.227 | 3.45E−97 | UTM1 | Pklr |
| Dnajb9 | 5.24E−101 | 0.27843915 | 0.463 | 0.122 | 1.30E−96 | UTM1 | Dnajb9 |
| Oaz1 | 1.23E−100 | 0.39502814 | 0.935 | 0.65 | 3.06E−96 | UTM1 | Oaz1 |
| Mbl2 | 4.21E−100 | 0.40599301 | 0.975 | 0.751 | 1.05E−95 | UTM1 | Mb12 |
| Manf | 2.64E−99 | 0.38367758 | 0.704 | 0.299 | 6.55E−95 | UTM1 | Manf |
| Acat3 | 7.89E−99 | 0.40666807 | 0.848 | 0.467 | 1.96E−94 | UTM1 | Acat3 |
| Etnppl | 1.87E−98 | 0.42088555 | 0.875 | 0.461 | 4.64E−94 | UTM1 | Etnppl |
| Plcxd2 | 1.39E−97 | 0.34692004 | 0.577 | 0.201 | 3.45E−93 | UTM1 | Plcxd2 |
| Ostc | 2.57E−97 | 0.36316755 | 0.604 | 0.225 | 6.39E−93 | UTM1 | Ostc |
| Pnpla7 | 2.61E−97 | 0.36692003 | 0.831 | 0.432 | 6.47E−93 | UTM1 | Pnpla7 |
| Hnmt | 3.66E−97 | 0.26396164 | 0.404 | 0.092 | 9.08E−93 | UTM1 | Hnmt |
| Eef1b2 | 9.52E−97 | 0.3987244 | 0.957 | 0.685 | 2.36E−92 | UTM1 | Eef1b2 |
| Abca1 | 2.41E−96 | 0.35753365 | 0.631 | 0.24 | 5.98E−92 | UTM1 | Abca1 |
| Fkbp3 | 9.55E−96 | 0.31527341 | 0.569 | 0.191 | 2.37E−91 | UTM1 | Fkbp3 |
| Atp5a1 | 7.99E−95 | 0.35267049 | 0.993 | 0.843 | 1.98E−90 | UTM1 | Atp5a1 |
| Aldh8a1 | 4.04E−93 | 0.384016 | 0.949 | 0.656 | 1.00E−88 | UTM1 | Aldh8a1 |
| 0610009D07Rik | 5.21E−93 | 0.30838393 | 0.497 | 0.151 | 1.29E−88 | UTM1 | 0610009D07Rik |
| Abca8a | 1.75E−92 | 0.27435964 | 0.505 | 0.155 | 4.33E−88 | UTM1 | Abca8a |
| Scfd1 | 5.18E−91 | 0.29620765 | 0.548 | 0.188 | 1.29E−86 | UTM1 | Scfd1 |
| Srebf1 | 7.07E−91 | 0.2732677 | 0.542 | 0.181 | 1.76E−86 | UTM1 | Srebf1 |
| Tmed7 | 8.15E−91 | 0.31475685 | 0.616 | 0.233 | 2.02E−86 | UTM1 | Tmed7 |
| Irf6 | 9.98E−91 | 0.28002315 | 0.414 | 0.107 | 2.48E−86 | UTM1 | Irf6 |
| Nr2f6 | 6.62E−90 | 0.36238079 | 0.69 | 0.296 | 1.64E−85 | UTM1 | Nr2f6 |
| Sigmar1 | 6.07E−89 | 0.37158458 | 0.765 | 0.38 | 1.51E−84 | UTM1 | Sigmar1 |
| 8430408G22Rik | 7.17E−89 | 0.33485641 | 0.641 | 0.258 | 1.78E−84 | UTM1 | 8430408G22Rik |
| Sppl2a | 4.27E−88 | 0.38180422 | 0.807 | 0.427 | 1.06E−83 | UTM1 | Sppl2a |
| C8g | 1.75E−87 | 0.34788408 | 0.997 | 0.863 | 4.34E−83 | UTM1 | C8g |
| BC089597 | 2.97E−87 | 0.36854001 | 0.743 | 0.351 | 7.38E−83 | UTM1 | BC089597 |
| Cmah | 6.65E−87 | 0.29941181 | 0.725 | 0.326 | 1.65E−82 | UTM1 | Cmah |
| Derl2 | 1.22E−86 | 0.34866072 | 0.753 | 0.363 | 3.02E−82 | UTM1 | Derl2 |
| Lypla1 | 1.79E−86 | 0.35084069 | 0.794 | 0.402 | 4.44E−82 | UTM1 | Lypla1 |
| Mettl9 | 2.28E−86 | 0.25341257 | 0.501 | 0.16 | 5.67E−82 | UTM1 | Mettl9 |
| Dhcr24 | 3.92E−86 | 0.4030962 | 0.954 | 0.692 | 9.73E−82 | UTM1 | Dhcr24 |
| Calm2 | 4.65E−86 | 0.320263 | 0.645 | 0.268 | 1.15E−81 | UTM1 | Calm2 |
| Cnpy2 | 2.44E−85 | 0.36416686 | 0.816 | 0.431 | 6.06E−81 | UTM1 | Cnpy2 |
| Commd6 | 3.71E−85 | 0.34118041 | 0.631 | 0.261 | 9.22E−81 | UTM1 | Commd6 |
| Chchd3 | 4.45E−85 | 0.34865152 | 0.774 | 0.385 | 1.10E−80 | UTM1 | Chchd3 |
| Hsd17b13 | 5.39E−85 | 0.37710065 | 0.939 | 0.634 | 1.34E−80 | UTM1 | Hsd17b13 |
| Pdhb | 1.32E−84 | 0.32631569 | 0.723 | 0.33 | 3.28E−80 | UTM1 | Pdhb |
| Sar1a | 1.56E−84 | 0.28167626 | 0.579 | 0.216 | 3.88E−80 | UTM1 | Sar1a |
| Rbbp4 | 2.11E−84 | 0.28766422 | 0.542 | 0.197 | 5.25E−80 | UTM1 | Rbbp4 |
| Cfh | 2.26E−84 | 0.32111506 | 0.987 | 0.848 | 5.61E−80 | UTM1 | Cfh |
| Hsd3b7 | 4.01E−84 | 0.38516037 | 0.961 | 0.71 | 9.96E−80 | UTM1 | Hsd3b7 |
| Sdha | 4.33E−84 | 0.37972281 | 0.938 | 0.645 | 1.08E−79 | UTM1 | Sdha |
| Ghr | 1.85E−83 | 0.35301973 | 0.971 | 0.76 | 4.59E−79 | UTM1 | Ghr |
| Tmem126a | 4.05E−83 | 0.30443005 | 0.552 | 0.203 | 1.01E−78 | UTM1 | Tmem126a |
| Cyc1 | 5.59E−83 | 0.39233732 | 0.911 | 0.587 | 1.39E−78 | UTM1 | Cyc1 |
| 1110058L19Rik | 2.22E−82 | 0.30158077 | 0.495 | 0.166 | 5.52E−78 | UTM1 | 1110058L19Rik |
| Sc4mol | 2.65E−82 | 0.38075178 | 0.876 | 0.516 | 6.58E−78 | UTM1 | Sc4mol |
| Ugt2a3 | 5.27E−82 | 0.37107252 | 0.947 | 0.68 | 1.31E−77 | UTM1 | Ugt2a3 |
| Commd3 | 1.87E−81 | 0.30521803 | 0.705 | 0.314 | 4.64E−77 | UTM1 | Commd3 |
| Atp6v0e | 2.87E−81 | 0.28872503 | 0.59 | 0.23 | 7.13E−77 | UTM1 | Atp6v0e |
| Spcs2 | 5.20E−81 | 0.34031177 | 0.759 | 0.382 | 1.29E−76 | UTM1 | Spcs2 |
| Ldlr | 6.47E−81 | 0.29459271 | 0.599 | 0.238 | 1.61E−76 | UTM1 | Ldlr |
| Psmb1 | 7.06E−81 | 0.38386195 | 0.934 | 0.665 | 1.75E−76 | UTM1 | Psmb1 |
| Mal2 | 1.03E−80 | 0.25266762 | 0.465 | 0.146 | 2.56E−76 | UTM1 | Mal2 |
| Ctnnb1 | 2.43E−80 | 0.27274635 | 0.68 | 0.305 | 6.04E−76 | UTM1 | Ctnnb1 |
| Tmem242 | 2.62E−80 | 0.25957867 | 0.531 | 0.188 | 6.50E−76 | UTM1 | Tmem242 |
| Nsdhl | 4.64E−80 | 0.31225962 | 0.639 | 0.27 | 1.15E−75 | UTM1 | Nsdhl |
| Hsd3b3 | 1.28E−79 | 0.35671743 | 0.839 | 0.479 | 3.18E−75 | UTM1 | Hsd3b3 |
| Dsg2 | 1.83E−79 | 0.32296016 | 0.721 | 0.343 | 4.54E−75 | UTM1 | Dsg2 |
| Prdx1 | 2.34E−79 | 0.34215053 | 0.991 | 0.893 | 5.81E−75 | UTM1 | Prdx1 |
| Ftcd | 1.91E−78 | 0.36144063 | 0.907 | 0.575 | 4.75E−74 | UTM1 | Ftcd |
| Adh5 | 2.70E−78 | 0.33120057 | 0.981 | 0.786 | 6.69E−74 | UTM1 | Adh5 |
| Elovl3 | 2.80E−78 | 0.3774663 | 0.979 | 0.746 | 6.95E−74 | UTM1 | Elovl3 |
| F2 | 3.01E−78 | 0.26845462 | 1 | 0.952 | 7.46E−74 | UTM1 | F2 |
| Chchd2 | 6.51E−78 | 0.32338065 | 0.987 | 0.878 | 1.62E−73 | UTM1 | Chchd2 |
| Bbox1 | 6.89E−78 | 0.35313715 | 0.789 | 0.419 | 1.71E−73 | UTM1 | Bbox1 |
| 1300002K09Rik | 1.92E−77 | 0.31041035 | 0.768 | 0.377 | 4.76E−73 | UTM1 | 1300002K09Rik |
| Khk | 2.02E−77 | 0.33758057 | 0.99 | 0.795 | 5.03E−73 | UTM1 | Khk |
| Cpb2 | 2.12E−77 | 0.32650203 | 0.969 | 0.73 | 5.27E−73 | UTM1 | Cpb2 |
| Psma1 | 1.03E−76 | 0.33310152 | 0.76 | 0.393 | 2.55E−72 | UTM1 | Psma1 |
| Iah1 | 3.92E−76 | 0.35199549 | 0.838 | 0.491 | 9.74E−72 | UTM1 | Iah1 |
| Gjb2 | 5.88E−76 | 0.32668553 | 0.815 | 0.442 | 1.46E−71 | UTM1 | Gjb2 |
| Selenbp2 | 6.78E−76 | 0.28572648 | 0.997 | 0.905 | 1.68E−71 | UTM1 | Selenbp2 |
| Agpat2 | 8.28E−76 | 0.33512398 | 0.872 | 0.513 | 2.06E−71 | UTM1 | Agpat2 |
| Pvrl3 | 2.24E−75 | 0.27299335 | 0.516 | 0.19 | 5.56E−71 | UTM1 | Pvrl3 |
| Slc25a15 | 4.50E−75 | 0.35215253 | 0.928 | 0.637 | 1.12E−70 | UTM1 | Slc25a15 |
| Ndufs1 | 9.39E−75 | 0.27648065 | 0.719 | 0.34 | 2.33E−70 | UTM1 | Ndufs1 |
| Tob1 | 1.26E−74 | 0.29859543 | 0.669 | 0.302 | 3.13E−70 | UTM1 | Tob1 |
| Etnk1 | 1.33E−74 | 0.26868318 | 0.547 | 0.21 | 3.31E−70 | UTM1 | Etnk1 |
| Cox7b | 1.74E−74 | 0.43393382 | 0.99 | 0.818 | 4.32E−70 | UTM1 | Cox7b |
| Ssr2 | 2.25E−74 | 0.30092095 | 0.698 | 0.33 | 5.58E−70 | UTM1 | Ssr2 |
| Ssu72 | 2.77E−74 | 0.30608708 | 0.726 | 0.359 | 6.89E−70 | UTM1 | Ssu72 |
| Ndufa4 | 4.03E−74 | 0.40135649 | 0.982 | 0.838 | 1.00E−69 | UTM1 | Ndufa4 |
| Cox7a2 | 4.67E−74 | 0.41356585 | 0.932 | 0.705 | 1.16E−69 | UTM1 | Cox7a2 |
| Ugt2b36 | 1.27E−73 | 0.3136071 | 0.997 | 0.894 | 3.16E−69 | UTM1 | Ugt2b36 |
| Pqlc1 | 3.16E−73 | 0.25791546 | 0.587 | 0.239 | 7.85E−69 | UTM1 | Pqlc1 |
| Rdh11 | 6.13E−73 | 0.28222183 | 0.571 | 0.23 | 1.52E−68 | UTM1 | Rdh11 |
| Prlr | 1.07E−72 | 0.25462979 | 0.531 | 0.201 | 2.65E−68 | UTM1 | Prlr |
| Uqcrb | 2.52E−72 | 0.35800773 | 0.843 | 0.532 | 6.27E−68 | UTM1 | Uqcrb |
| Laptm4a | 4.14E−72 | 0.30178581 | 0.817 | 0.462 | 1.03E−67 | UTM1 | Laptm4a |
| Tram1 | 5.55E−72 | 0.2883247 | 0.752 | 0.383 | 1.38E−67 | UTM1 | Tram1 |
| Ubl5 | 1.20E−71 | 0.28691562 | 0.609 | 0.262 | 2.98E−67 | UTM1 | Ubl5 |
| Copb1 | 1.49E−71 | 0.28426912 | 0.663 | 0.303 | 3.69E−67 | UTM1 | Copb1 |
| Deptor | 1.50E−71 | 0.26550315 | 0.72 | 0.341 | 3.72E−67 | UTM1 | Deptor |
| Rnf13 | 1.88E−71 | 0.29963446 | 0.752 | 0.39 | 4.67E−67 | UTM1 | Rnf13 |
| Cyp51 | 3.23E−71 | 0.34072444 | 0.874 | 0.528 | 8.03E−67 | UTM1 | Cyp51 |
| Uqcrfs1 | 4.97E−71 | 0.32365738 | 0.957 | 0.696 | 1.23E−66 | UTM1 | Uqcrfs1 |
| Atg3 | 6.60E−71 | 0.28289063 | 0.73 | 0.358 | 1.64E−66 | UTM1 | Atg3 |
| Apcs | 7.87E−71 | 0.33098607 | 0.767 | 0.395 | 1.95E−66 | UTM1 | Apcs |
| Apof | 8.05E−71 | 0.27900656 | 0.986 | 0.85 | 2.00E−66 | UTM1 | Apof |
| Arf4 | 3.11E−70 | 0.2516462 | 0.579 | 0.242 | 7.72E−66 | UTM1 | Arf4 |
| Tpi1 | 4.79E−70 | 0.31291177 | 0.907 | 0.598 | 1.19E−65 | UTM1 | Tpi1 |
| Sub1 | 9.00E−70 | 0.25980103 | 0.573 | 0.234 | 2.23E−65 | UTM1 | Sub1 |
| Serpina12 | 3.66E−69 | 0.30327542 | 0.727 | 0.365 | 9.08E−65 | UTM1 | Serpina12 |
| C8a | 4.69E−69 | 0.28701937 | 0.983 | 0.835 | 1.17E−64 | UTM1 | C8a |
| Dhcr7 | 5.00E−69 | 0.29409414 | 0.602 | 0.267 | 1.24E−64 | UTM1 | Dhcr7 |
| F13b | 1.25E−68 | 0.30662429 | 0.855 | 0.527 | 3.11E−64 | UTM1 | F13b |
| Lyrm5 | 1.27E−68 | 0.28597928 | 0.619 | 0.277 | 3.15E−64 | UTM1 | Lyrm5 |
| Ppp1r3b | 1.35E−68 | 0.25254489 | 0.604 | 0.26 | 3.34E−64 | UTM1 | Ppp1r3b |
| Ifitm3 | 1.73E−68 | 0.31398282 | 0.947 | 0.74 | 4.29E−64 | UTM1 | Ifitm3 |
| Hsd17b2 | 2.17E−68 | 0.31035164 | 0.908 | 0.606 | 5.39E−64 | UTM1 | Hsd17b2 |
| Rab10 | 2.97E−68 | 0.268356 | 0.647 | 0.289 | 7.37E−64 | UTM1 | Rab10 |
| Bud31 | 3.01E−68 | 0.26276769 | 0.636 | 0.285 | 7.47E−64 | UTM1 | Bud31 |
| Swi5 | 4.56E−68 | 0.29995649 | 0.736 | 0.375 | 1.13E−63 | UTM1 | Swi5 |
| Emc7 | 4.89E−68 | 0.27676545 | 0.721 | 0.365 | 1.22E−63 | UTM1 | Emc7 |
| Cltc | 4.95E−68 | 0.25557028 | 0.641 | 0.291 | 1.23E−63 | UTM1 | Cltc |
| Cct5 | 7.65E−68 | 0.27451127 | 0.782 | 0.421 | 1.90E−63 | UTM1 | Cct5 |
| Atf4 | 1.20E−67 | 0.27512816 | 0.671 | 0.319 | 2.99E−63 | UTM1 | Atf4 |
| Ndufb5 | 1.21E−67 | 0.33996987 | 0.826 | 0.497 | 3.01E−63 | UTM1 | Ndufb5 |
| Gm5096 | 1.43E−67 | 0.26079 | 0.701 | 0.343 | 3.55E−63 | UTM1 | Gm5096 |
| Perp | 2.15E−67 | 0.26670351 | 0.688 | 0.333 | 5.34E−63 | UTM1 | Perp |
| Rnf11 | 2.34E−67 | 0.2556776 | 0.717 | 0.346 | 5.81E−63 | UTM1 | Rnf11 |
| Ndufa5 | 2.84E−67 | 0.29341766 | 0.791 | 0.446 | 7.06E−63 | UTM1 | Ndufa5 |
| Serinc1 | 7.86E−67 | 0.30145323 | 0.84 | 0.478 | 1.95E−62 | UTM1 | Serinc1 |
| Cyp2j5 | 2.08E−66 | 0.28302319 | 0.979 | 0.825 | 5.16E−62 | UTM1 | Cyp2j5 |
| Fads1 | 2.27E−66 | 0.33006468 | 0.875 | 0.55 | 5.62E−62 | UTM1 | Fads1 |
| Sucla2 | 3.33E−66 | 0.29066376 | 0.771 | 0.422 | 8.28E−62 | UTM1 | Sucla2 |
| Pah | 8.49E−66 | 0.28093156 | 0.981 | 0.843 | 2.11E−61 | UTM1 | Pah |
| Sdhb | 9.55E−66 | 0.31480778 | 0.951 | 0.715 | 2.37E−61 | UTM1 | Sdhb |
| Cox4i1 | 1.20E−65 | 0.27616571 | 1 | 0.935 | 2.98E−61 | UTM1 | Cox4i1 |
| Pipox | 1.97E−65 | 0.2935053 | 0.776 | 0.427 | 4.90E−61 | UTM1 | Pipox |
| Baat | 4.65E−65 | 0.3059397 | 0.933 | 0.678 | 1.15E−60 | UTM1 | Baat |
| Ctsl | 5.37E−65 | 0.29836773 | 0.973 | 0.794 | 1.33E−60 | UTM1 | Ctsl |
| Prkd3 | 2.21E−64 | 0.29616642 | 0.713 | 0.371 | 5.49E−60 | UTM1 | Prkd3 |
| Akr1c20 | 1.15E−63 | 0.33238402 | 0.784 | 0.446 | 2.85E−59 | UTM1 | Akr1c20 |
| Evi5 | 1.49E−63 | 0.26301342 | 0.713 | 0.357 | 3.70E−59 | UTM1 | Evi5 |
| Mrps14 | 5.20E−63 | 0.26008108 | 0.569 | 0.246 | 1.29E−58 | UTM1 | Mrps14 |
| Sfxn1 | 1.09E−62 | 0.265142 | 0.737 | 0.367 | 2.70E−58 | UTM1 | Sfxn1 |
| Cyb5b | 1.38E−62 | 0.32175064 | 0.911 | 0.624 | 3.43E−58 | UTM1 | Cyb5b |
| Ccbl2 | 4.42E−62 | 0.28251976 | 0.801 | 0.451 | 1.10E−57 | UTM1 | Ccbl2 |
| Qprt | 7.81E−62 | 0.2899791 | 0.722 | 0.378 | 1.94E−57 | UTM1 | Qprt |
| Ube2b | 1.03E−61 | 0.27510318 | 0.704 | 0.356 | 2.57E−57 | UTM1 | Ube2b |
| Kynu | 1.28E−61 | 0.29239464 | 0.817 | 0.465 | 3.17E−57 | UTM1 | Kynu |
| Timm8b | 1.62E−61 | 0.29597925 | 0.747 | 0.406 | 4.03E−57 | UTM1 | Timm8b |
| Glud1 | 2.91E−61 | 0.26489254 | 0.989 | 0.86 | 7.22E−57 | UTM1 | Glud1 |
| Cnbp | 6.36E−61 | 0.29562084 | 0.947 | 0.687 | 1.58E−56 | UTM1 | Cnbp |
| Acss2 | 1.52E−60 | 0.30324766 | 0.808 | 0.449 | 3.78E−56 | UTM1 | Acss2 |
| Cml2 | 2.02E−60 | 0.26434013 | 0.972 | 0.795 | 5.02E−56 | UTM1 | Cml2 |
| Hal | 5.96E−60 | 0.32620613 | 0.933 | 0.651 | 1.48E−55 | UTM1 | Hal |
| Lgals9 | 1.05E−59 | 0.30869764 | 0.954 | 0.707 | 2.62E−55 | UTM1 | Lgals9 |
| 15-Sep | 1.54E−59 | 0.32295173 | 0.896 | 0.578 | 3.82E−55 | UTM1 | 15-Sep |
| Iigp1 | 2.59E−59 | 0.25498285 | 0.938 | 0.664 | 6.43E−55 | UTM1 | Iigp1 |
| Tlcd2 | 3.62E−59 | 0.28264137 | 0.765 | 0.413 | 8.98E−55 | UTM1 | Tlcd2 |
| Aldh1a7 | 6.57E−59 | 0.29599695 | 0.854 | 0.529 | 1.63E−54 | UTM1 | Aldh1a7 |
| Sar1b | 1.79E−58 | 0.30909884 | 0.943 | 0.68 | 4.45E−54 | UTM1 | Sar1b |
| Sdc2 | 2.74E−58 | 0.27931341 | 0.816 | 0.487 | 6.81E−54 | UTM1 | Sdc2 |
| Ugt2b5 | 3.75E−58 | 0.25716502 | 0.999 | 0.937 | 9.32E−54 | UTM1 | Ugt2b5 |
| Paics | 9.16E−58 | 0.28698914 | 0.865 | 0.553 | 2.27E−53 | UTM1 | Paics |
| Slc38a3 | 2.21E−57 | 0.28617356 | 0.971 | 0.797 | 5.48E−53 | UTM1 | Slc38a3 |
| Zbtb20 | 2.34E−57 | 0.26498064 | 0.668 | 0.324 | 5.81E−53 | UTM1 | Zbtb20 |
| Ghitm | 3.39E−57 | 0.27864994 | 0.92 | 0.639 | 8.42E−53 | UTM1 | Ghitm |
| Adk | 7.84E−57 | 0.27373253 | 0.979 | 0.796 | 1.95E−52 | UTM1 | Adk |
| Ssr3 | 4.83E−56 | 0.25203204 | 0.809 | 0.481 | 1.20E−51 | UTM1 | Ssr3 |
| Pygl | 5.88E−56 | 0.28584416 | 0.955 | 0.68 | 1.46E−51 | UTM1 | Pygl |
| Etfa | 4.41E−55 | 0.2569097 | 0.947 | 0.723 | 1.09E−50 | UTM1 | Etfa |
| Csad | 4.48E−55 | 0.34555095 | 0.902 | 0.659 | 1.11E−50 | UTM1 | Csad |
| Mrpl12 | 6.08E−55 | 0.27496438 | 0.799 | 0.467 | 1.51E−50 | UTM1 | Mrpl12 |
| Amdhd1 | 2.55E−54 | 0.28188607 | 0.87 | 0.549 | 6.33E−50 | UTM1 | Amdhd1 |
| C1s | 2.71E−54 | 0.25697751 | 0.897 | 0.582 | 6.73E−50 | UTM1 | C1s |
| Psma2 | 4.37E−54 | 0.27369868 | 0.744 | 0.428 | 1.09E−49 | UTM1 | Psma2 |
| S100a1 | 5.86E−54 | 0.29112411 | 0.858 | 0.555 | 1.45E−49 | UTM1 | S100a1 |
| Tm7sf2 | 6.73E−54 | 0.26535039 | 0.736 | 0.404 | 1.67E−49 | UTM1 | Tm7sf2 |
| Fdx1 | 1.81E−53 | 0.27825732 | 0.823 | 0.518 | 4.50E−49 | UTM1 | Fdx1 |
| Adck3 | 4.36E−53 | 0.282871 | 0.948 | 0.665 | 1.08E−48 | UTM1 | Adck3 |
| Hsd17b12 | 7.99E−53 | 0.25465919 | 0.85 | 0.539 | 1.98E−48 | UTM1 | Hsd17b12 |
| Mcfd2 | 3.47E−52 | 0.26216867 | 0.857 | 0.529 | 8.63E−48 | UTM1 | Mcfd2 |
| Ces1f | 3.57E−52 | 0.27147075 | 0.955 | 0.744 | 8.87E−48 | UTM1 | Ces1f |
| Hebp1 | 1.89E−49 | 0.26932242 | 0.913 | 0.662 | 4.70E−45 | UTM1 | Hebp1 |
| Gnb2l1 | 3.81E−49 | 0.27040884 | 0.963 | 0.751 | 9.46E−45 | UTM1 | Gnb2l1 |
| Ndufb8 | 7.67E−48 | 0.26808735 | 0.912 | 0.657 | 1.91E−43 | UTM1 | Ndufb8 |
| Ugt3a2 | 1.72E−47 | 0.25614571 | 0.879 | 0.587 | 4.28E−43 | UTM1 | Ugt3a2 |
| Tfr2 | 8.70E−47 | 0.25214071 | 0.832 | 0.526 | 2.16E−42 | UTM1 | Tfr2 |
| Tmprss6 | 2.49E−44 | 0.25597646 | 0.914 | 0.643 | 6.18E−40 | UTM1 | Tmprss6 |
| Cyp2b101 | 0 | 1.25971488 | 0.913 | 0.272 | 0 | UTM5 | Cyp2b10 |
| Gm239351 | 0 | 1.13877874 | 0.999 | 0.871 | 0 | UTM5 | Gm23935 |
| Ttr | 0 | 0.46574445 | 1 | 0.999 | 0 | UTM5 | Ttr |
| Apoe | 0 | 0.43350247 | 1 | 1 | 0 | UTM5 | Apoe |
| Hpd1 | 8.94E−267 | 0.55870766 | 0.999 | 0.96 | 2.22E−262 | UTM5 | Hpd |
| Ankrd551 | 2.77E−259 | 1.04657324 | 0.972 | 0.494 | 6.87E−255 | UTM5 | Ankrd55 |
| Cyb5 | 4.10E−251 | 0.54320014 | 1 | 0.971 | 1.02E−246 | UTM5 | Cyb5 |
| Saa11 | 5.70E−245 | 0.91748275 | 0.905 | 0.484 | 1.42E−240 | UTM5 | Saa1 |
| Gstm11 | 7.92E−243 | 0.76000515 | 0.998 | 0.89 | 1.97E−238 | UTM5 | Gstm1 |
| Gstm31 | 3.48E−236 | 1.08145502 | 0.699 | 0.207 | 8.63E−232 | UTM5 | Gstm3 |
| Mt11 | 5.28E−234 | 0.89508131 | 0.822 | 0.362 | 1.31E−229 | UTM5 | Mt1 |
| Vtn | 1.81E−219 | 0.48408458 | 1 | 0.97 | 4.50E−215 | UTM5 | Vtn |
| Cyp2c291 | 2.49E−215 | 0.81028694 | 0.972 | 0.899 | 6.19E−211 | UTM5 | Cyp2c29 |
| Cyp2a51 | 1.92E−211 | 1.0797726 | 0.822 | 0.361 | 4.76E−207 | UTM5 | Cyp2a5 |
| Apoa21 | 1.29E−202 | 0.28295599 | 1 | 1 | 3.21E−198 | UTM5 | Apoa2 |
| Apoa11 | 1.97E−200 | 0.28070646 | 1 | 1 | 4.90E−196 | UTM5 | Apoa1 |
| Mt21 | 1.41E−195 | 0.72818605 | 0.636 | 0.168 | 3.50E−191 | UTM5 | Mt2 |
| Hp1 | 6.85E−185 | 0.32448757 | 1 | 0.997 | 1.70E−180 | UTM5 | Hp |
| Atp5h1 | 7.01E−179 | 0.67755519 | 0.867 | 0.547 | 1.74E−174 | UTM5 | Atp5h |
| Gpx1 | 3.55E−154 | 0.28180291 | 1 | 0.997 | 8.81E−150 | UTM5 | Gpx1 |
| Chchd101 | 4.15E−148 | 0.447182 | 0.991 | 0.91 | 1.03E−143 | UTM5 | Chchd10 |
| Adh1 | 5.65E−144 | 0.39999808 | 0.999 | 0.932 | 1.40E−139 | UTM5 | Adh1 |
| Aldh1a11 | 6.03E−143 | 0.4817687 | 0.997 | 0.926 | 1.50E−138 | UTM5 | Aldh1a1 |
| Lrg11 | 3.27E−142 | 0.55473352 | 0.877 | 0.633 | 8.11E−138 | UTM5 | Lrg1 |
| Gdf151 | 1.02E−137 | 0.61534668 | 0.58 | 0.216 | 2.54E−133 | UTM5 | Gdf15 |
| Rnase41 | 2.68E−137 | 0.51253382 | 0.99 | 0.871 | 6.65E−133 | UTM5 | Rnase4 |
| Hpx | 4.74E−135 | 0.38094396 | 1 | 0.995 | 1.18E−130 | UTM5 | Hpx |
| Apoc4 | 2.97E−132 | 0.37324314 | 0.999 | 0.966 | 7.37E−128 | UTM5 | Apoc4 |
| Cyp2e11 | 1.64E−130 | 0.61429276 | 0.976 | 0.864 | 4.07E−126 | UTM5 | Cyp2e1 |
| Serping11 | 4.00E−130 | 0.52201124 | 0.96 | 0.804 | 9.94E−126 | UTM5 | Serping1 |
| Id31 | 1.50E−127 | 0.62698859 | 0.629 | 0.278 | 3.72E−123 | UTM5 | Id3 |
| Cyp4a101 | 4.32E−127 | 0.56062409 | 0.829 | 0.531 | 1.07E−122 | UTM5 | Cyp4a10 |
| Stard101 | 3.57E−123 | 0.34738438 | 0.999 | 0.944 | 8.86E−119 | UTM5 | Stard10 |
| Cyp2d91 | 1.16E−120 | 0.36911806 | 0.999 | 0.93 | 2.88E−116 | UTM5 | Cyp2d9 |
| Cyp1a21 | 3.03E−120 | 0.59697802 | 0.919 | 0.702 | 7.52E−116 | UTM5 | Cyp1a2 |
| Cebpb1 | 1.84E−115 | 0.52562681 | 0.644 | 0.309 | 4.57E−111 | UTM5 | Cebpb |
| Cyp2c501 | 3.03E−114 | 0.51306773 | 0.964 | 0.832 | 7.53E−110 | UTM5 | Cyp2c50 |
| Serpina1a1 | 4.36E−113 | 0.29692434 | 1 | 0.984 | 1.08E−108 | UTM5 | Serpina1a |
| Gstp11 | 2.22E−107 | 0.35098945 | 0.953 | 0.726 | 5.51E−103 | UTM5 | Gstp1 |
| C4b1 | 3.15E−107 | 0.44347886 | 0.958 | 0.828 | 7.81E−103 | UTM5 | C4b |
| Mug11 | 5.94E−104 | 0.29868649 | 0.999 | 0.982 | 1.47E−99 | UTM5 | Mug1 |
| Ttc361 | 6.13E−102 | 0.35591693 | 0.992 | 0.919 | 1.52E−97 | UTM5 | Ttc36 |
| Gadd45b1 | 1.96E−98 | 0.55034957 | 0.466 | 0.163 | 4.87E−94 | UTM5 | Gadd45b |
| Gsta3 | 9.99E−98 | 0.37015533 | 0.999 | 0.925 | 2.48E−93 | UTM5 | Gsta3 |
| 1-Jun | 9.07E−96 | 0.52377024 | 0.737 | 0.488 | 2.25E−91 | UTM5 | Jun |
| 1100001G20Rik1 | 1.92E−93 | 0.30823546 | 0.999 | 0.97 | 4.76E−89 | UTM5 | 1100001G20Rik |
| Ces1c | 3.51E−90 | 0.26725555 | 1 | 0.974 | 8.73E−86 | UTM5 | Ces1c |
| Cyp27a1 | 8.39E−87 | 0.43363589 | 0.937 | 0.764 | 2.08E−82 | UTM5 | Cyp27a1 |
| Fos1 | 4.46E−84 | 0.54575752 | 0.655 | 0.404 | 1.11E−79 | UTM5 | Fos |
| Sephs21 | 6.09E−83 | 0.41975136 | 0.866 | 0.684 | 1.51E−78 | UTM5 | Sephs2 |
| Itih4 | 3.62E−81 | 0.34372846 | 0.992 | 0.916 | 8.98E−77 | UTM5 | Itih4 |
| Rps151 | 7.09E−81 | 0.37609074 | 0.685 | 0.414 | 1.76E−76 | UTM5 | Rps15 |
| Pim31 | 7.78E−81 | 0.44118967 | 0.675 | 0.436 | 1.93E−76 | UTM5 | Pim3 |
| Ephx11 | 1.29E−80 | 0.41078893 | 0.888 | 0.736 | 3.20E−76 | UTM5 | Ephx1 |
| Clu | 2.42E−80 | 0.31373997 | 0.995 | 0.93 | 6.01E−76 | UTM5 | Clu |
| Por1 | 2.09E−78 | 0.35994138 | 0.813 | 0.575 | 5.19E−74 | UTM5 | Por |
| Sult1d11 | 4.93E−78 | 0.45000439 | 0.806 | 0.594 | 1.22E−73 | UTM5 | Sult1d1 |
| Junb1 | 1.08E−77 | 0.45123021 | 0.614 | 0.365 | 2.69E−73 | UTM5 | Junb |
| Usmg51 | 2.84E−76 | 0.42129894 | 0.587 | 0.316 | 7.06E−72 | UTM5 | Usmg5 |
| Cxcl1 | 6.62E−76 | 0.48969282 | 0.46 | 0.208 | 1.64E−71 | UTM5 | Cxcl1 |
| mt-Nd41 | 5.60E−75 | 0.38334466 | 0.999 | 0.997 | 1.39E−70 | UTM5 | mt-Nd4 |
| Ahcy1 | 1.92E−74 | 0.31198715 | 0.978 | 0.871 | 4.77E−70 | UTM5 | Ahcy |
| Lcat1 | 3.72E−74 | 0.38055835 | 0.873 | 0.714 | 9.23E−70 | UTM5 | Lcat |
| Fth1 | 9.59E−74 | 0.25419048 | 0.999 | 0.963 | 2.38E−69 | UTM5 | Fth1 |
| Clpx | 2.75E−73 | 0.41799583 | 0.9 | 0.698 | 6.82E−69 | UTM5 | Clpx |
| Grhpr1 | 6.45E−72 | 0.37056505 | 0.868 | 0.705 | 1.60E−67 | UTM5 | Grhpr |
| mt-Co11 | 1.16E−71 | 0.3699118 | 0.999 | 0.999 | 2.88E−67 | UTM5 | mt-Co1 |
| Gchfr | 9.17E−69 | 0.40305281 | 0.871 | 0.692 | 2.28E−64 | UTM5 | Gchfr |
| Rpl351 | 3.15E−68 | 0.31190843 | 0.367 | 0.121 | 7.83E−64 | UTM5 | Rpl35 |
| Cyp2c551 | 6.39E−68 | 0.42673384 | 0.382 | 0.142 | 1.59E−63 | UTM5 | Cyp2c55 |
| Cox8a1 | 1.45E−67 | 0.37035824 | 0.735 | 0.537 | 3.60E−63 | UTM5 | Cox8a |
| Eef1a11 | 1.55E−65 | 0.25671984 | 0.934 | 0.788 | 3.86E−61 | UTM5 | Eef1a1 |
| Insig2 | 1.40E−64 | 0.36315872 | 0.907 | 0.739 | 3.47E−60 | UTM5 | Insig2 |
| Ces2a1 | 6.29E−64 | 0.32781496 | 0.918 | 0.792 | 1.56E−59 | UTM5 | Ces2a |
| Gulo1 | 7.25E−64 | 0.46673554 | 0.629 | 0.401 | 1.80E−59 | UTM5 | Gulo |
| Haao | 1.02E−62 | 0.34164895 | 0.952 | 0.794 | 2.53E−58 | UTM5 | Haao |
| Cd302 | 1.20E−62 | 0.34750482 | 0.941 | 0.801 | 2.98E−58 | UTM5 | Cd302 |
| Slc25a471 | 6.73E−62 | 0.30668037 | 0.976 | 0.891 | 1.67E−57 | UTM5 | Slc25a47 |
| Mettl7b1 | 1.12E−60 | 0.29547032 | 0.939 | 0.819 | 2.79E−56 | UTM5 | Mettl7b |
| Serpina3n1 | 3.29E−60 | 0.36591685 | 0.686 | 0.492 | 8.17E−56 | UTM5 | Serpina3n |
| Saa21 | 5.98E−58 | 0.34882206 | 0.354 | 0.126 | 1.49E−53 | UTM5 | Saa2 |
| Ehhadh1 | 2.11E−57 | 0.3315863 | 0.793 | 0.616 | 5.25E−53 | UTM5 | Ehhadh |
| Ttpa | 9.77E−57 | 0.30846742 | 0.941 | 0.816 | 2.43E−52 | UTM5 | Ttpa |
| Tmem176b | 1.05E−56 | 0.39429345 | 0.733 | 0.558 | 2.61E−52 | UTM5 | Tmem176b |
| Cyp2c541 | 3.08E−56 | 0.35166996 | 0.856 | 0.688 | 7.65E−52 | UTM5 | Cyp2c54 |
| Creg1 | 5.60E−54 | 0.27676691 | 0.977 | 0.888 | 1.39E−49 | UTM5 | Creg1 |
| Blvrb1 | 5.56E−53 | 0.35628811 | 0.671 | 0.486 | 1.38E−48 | UTM5 | Blvrb |
| mt-Nd51 | 3.78E−52 | 0.30458553 | 0.997 | 0.994 | 9.39E−48 | UTM5 | mt-Nd5 |
| Ang | 4.85E−52 | 0.34485878 | 0.791 | 0.61 | 1.20E−47 | UTM5 | Ang |
| Pcbd1 | 7.00E−52 | 0.30897079 | 0.926 | 0.772 | 1.74E−47 | UTM5 | Pcbd1 |
| Gstm4 | 8.03E−52 | 0.39950266 | 0.557 | 0.361 | 1.99E−47 | UTM5 | Gstm4 |
| Hmgcl | 1.77E−51 | 0.34120969 | 0.814 | 0.667 | 4.38E−47 | UTM5 | Hmgcl |
| Fgl11 | 2.73E−51 | 0.28479212 | 0.866 | 0.725 | 6.78E−47 | UTM5 | Fgl1 |
| Sqstm11 | 4.59E−51 | 0.30323927 | 0.891 | 0.743 | 1.14E−46 | UTM5 | Sqstm1 |
| Rarres2 | 2.72E−48 | 0.270449 | 0.969 | 0.858 | 6.75E−44 | UTM5 | Rarres2 |
| Cyp2c371 | 3.23E−48 | 0.35466642 | 0.725 | 0.523 | 8.02E−44 | UTM5 | Cyp2c37 |
| Ier2 | 4.25E−47 | 0.36177787 | 0.595 | 0.413 | 1.05E−42 | UTM5 | Ier2 |
| Ndufs8 | 3.46E−46 | 0.32706621 | 0.704 | 0.537 | 8.59E−42 | UTM5 | Ndufs8 |
| Abcc31 | 1.11E−45 | 0.33711807 | 0.643 | 0.467 | 2.76E−41 | UTM5 | Abcc3 |
| Slc22a11 | 1.81E−45 | 0.34693238 | 0.824 | 0.691 | 4.49E−41 | UTM5 | Slc22a1 |
| Gstt31 | 6.29E−45 | 0.28628594 | 0.403 | 0.201 | 1.56E−40 | UTM5 | Gstt3 |
| Hrg | 1.33E−44 | 0.29365973 | 0.911 | 0.773 | 3.29E−40 | UTM5 | Hrg |
| Rnf125 | 5.51E−44 | 0.35260939 | 0.59 | 0.422 | 1.37E−39 | UTM5 | Rnf125 |
| F10 | 7.41E−44 | 0.26087857 | 0.958 | 0.848 | 1.84E−39 | UTM5 | F10 |
| Decr21 | 1.03E−43 | 0.26539891 | 0.726 | 0.567 | 2.56E−39 | UTM5 | Decr2 |
| Nr1i31 | 1.14E−43 | 0.30480484 | 0.495 | 0.291 | 2.82E−39 | UTM5 | Nr1i3 |
| Arrdc3 | 6.96E−43 | 0.30193418 | 0.317 | 0.136 | 1.73E−38 | UTM5 | Arrdc3 |
| Lect21 | 7.92E−43 | 0.32510938 | 0.526 | 0.332 | 1.97E−38 | UTM5 | Lect2 |
| 4931406C07Rik1 | 6.05E−42 | 0.26266358 | 0.822 | 0.694 | 1.50E−37 | UTM5 | 4931406C07Rik |
| Zfp36 | 3.34E−40 | 0.34031672 | 0.616 | 0.453 | 8.29E−36 | UTM5 | Zfp36 |
| Gstm2 | 4.91E−40 | 0.30094499 | 0.263 | 0.101 | 1.22E−35 | UTM5 | Gstm2 |
| Ndufa7 | 1.62E−39 | 0.28033658 | 0.804 | 0.655 | 4.01E−35 | UTM5 | Ndufa7 |
| Selenbp1 | 1.24E−38 | 0.31122695 | 0.779 | 0.64 | 3.07E−34 | UTM5 | Selenbp1 |
| Akr1c14 | 1.49E−38 | 0.29991907 | 0.796 | 0.642 | 3.70E−34 | UTM5 | Akr1c14 |
| Cdkn1a | 1.01E−37 | 0.31716116 | 0.288 | 0.127 | 2.51E−33 | UTM5 | Cdkn1a |
| Cyp4f151 | 1.32E−37 | 0.28293592 | 0.529 | 0.355 | 3.27E−33 | UTM5 | Cyp4f15 |
| Slc27a5 | 3.39E−37 | 0.25311303 | 0.967 | 0.839 | 8.41E−33 | UTM5 | Slc27a5 |
| Grn1 | 3.92E−37 | 0.31937969 | 0.49 | 0.327 | 9.74E−33 | UTM5 | Grn |
| Jund1 | 1.07E−36 | 0.26166892 | 0.402 | 0.227 | 2.66E−32 | UTM5 | Jund |
| Papss2 | 4.28E−36 | 0.32662518 | 0.592 | 0.446 | 1.06E−31 | UTM5 | Papss2 |
| Tmem14c | 1.87E−35 | 0.27112198 | 0.668 | 0.524 | 4.66E−31 | UTM5 | Tmem14c |
| Serpinf2 | 9.46E−35 | 0.25152649 | 0.932 | 0.778 | 2.35E−30 | UTM5 | Serpinf2 |
| Atf3 | 1.61E−34 | 0.26673538 | 0.268 | 0.115 | 3.99E−30 | UTM5 | Atf3 |
| Car5a | 1.23E−33 | 0.27823285 | 0.426 | 0.261 | 3.05E−29 | UTM5 | Car5a |
| Aldh3a21 | 1.96E−33 | 0.28055406 | 0.627 | 0.457 | 4.87E−29 | UTM5 | Aldh3a2 |
| Gclc | 2.02E−33 | 0.27548506 | 0.81 | 0.652 | 5.02E−29 | UTM5 | Gclc |
| Oaf | 2.11E−33 | 0.26638374 | 0.875 | 0.736 | 5.23E−29 | UTM5 | Oaf |
| Apoc2 | 3.74E−33 | 0.26484153 | 0.874 | 0.749 | 9.28E−29 | UTM5 | Apoc2 |
| Gpld1 | 4.73E−33 | 0.27600756 | 0.656 | 0.519 | 1.17E−28 | UTM5 | Gpld1 |
| Fcgrt | 1.15E−32 | 0.26729682 | 0.67 | 0.548 | 2.85E−28 | UTM5 | Fcgrt |
| Os91 | 2.32E−32 | 0.27254554 | 0.74 | 0.607 | 5.75E−28 | UTM5 | Os9 |
| Egr1 | 4.39E−31 | 0.30443562 | 0.534 | 0.383 | 1.09E−26 | UTM5 | Egr1 |
| AI182371 | 3.39E−30 | 0.25241508 | 0.7 | 0.565 | 8.43E−26 | UTM5 | AI182371 |
| Dcxr | 1.15E−29 | 0.25873551 | 0.702 | 0.579 | 2.87E−25 | UTM5 | Dcxr |
| Mvd | 3.99E−24 | 0.26099975 | 0.344 | 0.214 | 9.91E−20 | UTM5 | Mvd |
| Hsbp1 | 6.04E−23 | 0.25129426 | 0.56 | 0.453 | 1.50E−18 | UTM5 | Hsbp1 |
| Gstm6 | 2.26E−19 | 0.25216132 | 0.418 | 0.303 | 5.61E−15 | UTM5 | Gstm6 |
| Mup62 | 0 | 2.1978265 | 1 | 0.99 | 0 | UTM6 | Mup6 |
| Mup192 | 0 | 1.93347001 | 1 | 1 | 0 | UTM6 | Mup19 |
| Mup52 | 0 | 1.84833854 | 0.956 | 0.804 | 0 | UTM6 | Mup5 |
| Mup92 | 0 | 1.72069868 | 1 | 0.969 | 0 | UTM6 | Mup9 |
| Mup42 | 0 | 1.70689156 | 0.977 | 0.875 | 0 | UTM6 | Mup4 |
| Mup182 | 0 | 1.70527221 | 1 | 0.996 | 0 | UTM6 | Mup18 |
| Mup72 | 0 | 1.47233248 | 1 | 0.997 | 0 | UTM6 | Mup7 |
| Mup171 | 6.34E−294 | 1.18231635 | 1 | 1 | 1.57E−289 | UTM6 | Mup17 |
| Mup111 | 1.10E−284 | 0.88902183 | 1 | 1 | 2.72E−280 | UTM6 | Mup11 |
| Mup152 | 4.14E−269 | 1.73856185 | 0.882 | 0.538 | 1.03E−264 | UTM6 | Mup15 |
| Zfp683 | 1.40E−265 | 1.15363136 | 0.464 | 0.013 | 3.48E−261 | UTM6 | Zfp683 |
| mmu-mir-62362 | 2.75E−253 | 1.74996425 | 0.937 | 0.807 | 6.84E−249 | UTM6 | mmu-mir-6236 |
| Mup202 | 2.90E−223 | 0.88182043 | 1 | 1 | 7.21E−219 | UTM6 | Mup20 |
| Mup112 | 2.80E−219 | 1.50547218 | 0.832 | 0.51 | 6.96E−215 | UTM6 | Mup1 |
| Mup102 | 1.09E−218 | 1.51308329 | 0.855 | 0.644 | 2.70E−214 | UTM6 | Mup10 |
| mt-Co21 | 1.01E−209 | 1.54504766 | 0.85 | 0.563 | 2.51E−205 | UTM6 | mt-Co2 |
| Mup162 | 3.06E−209 | 1.07242857 | 0.941 | 0.932 | 7.59E−205 | UTM6 | Mup16 |
| Malat11 | 1.37E−207 | 1.32741509 | 0.909 | 0.742 | 3.41E−203 | UTM6 | Malat1 |
| Mup141 | 3.97E−190 | 1.32922546 | 0.817 | 0.556 | 9.87E−186 | UTM6 | Mup14 |
| Mup212 | 2.78E−182 | 0.86449671 | 0.943 | 0.924 | 6.90E−178 | UTM6 | Mup21 |
| Mup82 | 3.15E−165 | 1.37369891 | 0.668 | 0.285 | 7.83E−161 | UTM6 | Mup8 |
| Lars21 | 9.42E−162 | 0.76849968 | 0.99 | 0.996 | 2.34E−157 | UTM6 | Lars2 |
| Mup122 | 4.34E−151 | 0.93855833 | 0.875 | 0.855 | 1.08E−146 | UTM6 | Mup12 |
| mt-Atp62 | 2.51E−137 | 1.29404948 | 0.686 | 0.383 | 6.23E−133 | UTM6 | mt-Atp6 |
| mt-Co31 | 4.13E−117 | 1.09991599 | 0.762 | 0.555 | 1.03E−112 | UTM6 | mt-Co3 |
| Mup23 | 1.11E−113 | 1.13035871 | 0.616 | 0.326 | 2.75E−109 | UTM6 | Mup2 |
| Gm205942 | 7.76E−113 | 1.04926452 | 0.549 | 0.219 | 1.93E−108 | UTM6 | Gm20594 |
| Gm269241 | 3.84E−107 | 0.56665866 | 1 | 1 | 9.53E−103 | UTM6 | Gm26924 |
| Gm137751 | 9.06E−88 | 0.83647132 | 0.772 | 0.757 | 2.25E−83 | UTM6 | Gm13775 |
| Gstp12 | 9.87E−87 | 0.68055321 | 0.871 | 0.807 | 2.45E−82 | UTM6 | Gstp1 |
| Rpl412 | 2.55E−78 | 0.95632944 | 0.482 | 0.218 | 6.34E−74 | UTM6 | Rpl41 |
| Scp21 | 5.17E−76 | 0.42678693 | 0.952 | 0.996 | 1.28E−71 | UTM6 | Scp2 |
| mt-Rnr1 | 3.95E−68 | 0.42177683 | 1 | 1 | 9.81E−64 | UTM6 | mt-Rnr1 |
| Sparcl1 | 6.00E−68 | 0.60925972 | 0.308 | 0.085 | 1.49E−63 | UTM6 | Sparcl1 |
| Ftl12 | 1.01E−56 | 0.86494473 | 0.537 | 0.371 | 2.51E−52 | UTM6 | Ftl1 |
| Hamp1 | 1.07E−54 | 0.53431054 | 0.835 | 0.899 | 2.65E−50 | UTM6 | Hamp |
| Gm155641 | 6.11E−52 | 0.49223563 | 0.9 | 0.972 | 1.52E−47 | UTM6 | Gm15564 |
| Gm242451 | 6.15E−44 | 0.67966077 | 0.36 | 0.171 | 1.53E−39 | UTM6 | Gm24245 |
| Cyp3a44 | 8.24E−41 | 0.5893919 | 0.323 | 0.142 | 2.05E−36 | UTM6 | Cyp3a44 |
| Gm4076 | 2.95E−36 | 0.61348099 | 0.314 | 0.146 | 7.33E−32 | UTM6 | Gm4076 |
| Mup131 | 1.47E−32 | 0.63253389 | 0.59 | 0.533 | 3.65E−28 | UTM6 | Mup13 |
| mt-Nd52 | 6.68E−32 | 0.37550761 | 0.988 | 0.997 | 1.66E−27 | UTM6 | mt-Nd5 |
| Hsp90b11 | 8.04E−29 | 0.37916528 | 0.888 | 0.972 | 2.00E−24 | UTM6 | Hsp90b1 |
| Eef1a12 | 7.93E−27 | 0.42249546 | 0.746 | 0.881 | 1.97E−22 | UTM6 | Eef1a1 |
| Cyp2e12 | 5.38E−26 | 0.39331803 | 0.897 | 0.915 | 1.34E−21 | UTM6 | Cyp2e1 |
| mt-Nd2 | 6.69E−23 | 0.29590227 | 0.996 | 1 | 1.66E−18 | UTM6 | mt-Nd2 |
| Rn7sk | 3.38E−22 | 0.53767783 | 0.283 | 0.157 | 8.38E−18 | UTM6 | Rn7sk |
| Cfb1 | 1.06E−20 | 0.51119164 | 0.564 | 0.57 | 2.63E−16 | UTM6 | Cfb |
| Ubb1 | 1.76E−20 | 0.62082198 | 0.485 | 0.439 | 4.36E−16 | UTM6 | Ubb |
| Rps181 | 5.88E−18 | 0.58975018 | 0.555 | 0.585 | 1.46E−13 | UTM6 | Rps18 |
| Acaa1a1 | 1.88E−15 | 0.47992175 | 0.602 | 0.702 | 4.66E−11 | UTM6 | Acaa1a |
| Acsl1 | 1.45E−13 | 0.29198038 | 0.812 | 0.958 | 3.59E−09 | UTM6 | Acsl1 |
| Rplp1 | 1.81E−11 | 0.38988375 | 0.644 | 0.791 | 4.50E−07 | UTM6 | Rplp1 |
| Rps3a11 | 2.43E−11 | 0.51647276 | 0.373 | 0.324 | 6.03E−07 | UTM6 | Rps3a1 |
| Rps22 | 1.83E−10 | 0.39471423 | 0.638 | 0.792 | 4.55E−06 | UTM6 | Rps2 |
| mt-Nd61 | 6.18E−10 | 0.53191088 | 0.531 | 0.591 | 1.53E−05 | UTM6 | mt-Nd6 |
| Rps191 | 7.08E−10 | 0.56897855 | 0.46 | 0.478 | 1.76E−05 | UTM6 | Rps19 |
| Rpl371 | 3.50E−09 | 0.44536423 | 0.28 | 0.221 | 8.70E−05 | UTM6 | Rpl37 |
| Gm98431 | 4.44E−09 | 0.49576113 | 0.333 | 0.289 | 0.00011029 | UTM6 | Gm9843 |
| Cyp7b11 | 6.85E−08 | 0.34003655 | 0.65 | 0.788 | 0.00170044 | UTM6 | Cyp7b1 |
| Txnip1 | 2.08E−07 | 0.46854072 | 0.328 | 0.288 | 0.00515553 | UTM6 | Txnip |
| Acadm | 3.44E−07 | 0.41172686 | 0.593 | 0.768 | 0.00854147 | UTM6 | Acadm |
| Por2 | 4.81E−07 | 0.40526153 | 0.586 | 0.702 | 0.01194371 | UTM6 | Por |
| Rpl35a | 7.34E−07 | 0.50694911 | 0.373 | 0.364 | 0.01823153 | UTM6 | Rpl35a |
| Cyp4a141 | 9.89E−07 | 0.49263055 | 0.419 | 0.434 | 0.02454877 | UTM6 | Cyp4a14 |
| Rpl261 | 1.34E−06 | 0.40870741 | 0.485 | 0.544 | 0.03335525 | UTM6 | Rpl26 |
| St3gal51 | 1.55E−06 | 0.47782408 | 0.46 | 0.499 | 0.03844786 | UTM6 | St3gal5 |
| Plin2 | 8.88E−06 | 0.39991811 | 0.584 | 0.753 | 0.22063007 | UTM6 | Plin2 |
| Atp5e1 | 9.71E−05 | 0.30778372 | 0.69 | 0.875 | 1 | UTM6 | Atp5e |
| Rpl71 | 9.76E−05 | 0.3671681 | 0.257 | 0.226 | 1 | UTM6 | Rpl7 |
| Rps91 | 0.00010397 | 0.39135344 | 0.557 | 0.71 | 1 | UTM6 | Rps9 |
| Gm102501 | 0.00071118 | 0.35509478 | 0.266 | 0.246 | 1 | UTM6 | Gm10250 |
| Epb4.1 | 0.00164191 | 0.26962248 | 0.224 | 0.328 | 1 | UTM6 | Epb4.1 |
| Rpl36al1 | 0.00170775 | 0.40245344 | 0.472 | 0.575 | 1 | UTM6 | Rpl36al |
| Pdap1 | 0.00208461 | 0.26844074 | 0.239 | 0.354 | 1 | UTM6 | Pdap1 |
| Cox6b1 | 0.00306562 | 0.26517687 | 0.656 | 0.855 | 1 | UTM6 | Cox6b1 |
| Alas11 | 0.00687451 | 0.28080817 | 0.6 | 0.767 | 1 | UTM6 | Alas1 |
| Rps201 | 0.00787049 | 0.37390189 | 0.49 | 0.613 | 1 | UTM6 | Rps20 |
| Eif1 | 0.00888028 | 0.36530092 | 0.409 | 0.481 | 1 | UTM6 | Eif1 |

While hepatocytes from each untreated mouse clustered independently, the injury samples grouped by time point and injury type, rather than mouse of origin, indicating that the transcriptional response to injury causes individual hepatocytes to become more similar to one another. To confirm that this clustering captures biological, rather than technical, variation, Applicants performed differential expression to identify genes unique to each cluster. Clusters were defined by many genes related to liver function, injury response, and oxidative stress (FIG. 18D, see, e.g. Tables 2-5c), and technical gradients led to variation within, rather than across, clusters (nGene, nUMI; FIG. 25A-25H). Regression over technical variables (i.e., number of genes) largely removed these technical gradients, but preserved other, biologically important signals; removal of PC1, which captured technical effects, similarly resulted in a reduction of technical signals while preserving key biological ones. Since regression changed very little, other than downweighting technical differences in cell quality, and the biological signals on which this work focuses were robust to regression, Applicants opted to use the non-regressed dataset in our downstream analysis to avoid possible introduction of artificial variation.

APAP injury resulted in pericentral necrosis after 6 hrs as demonstrated by histological analysis (hereafter A6; FIG. 18B, 18C). Hepatocytes scoring high for a pericentral hepatocyte signature (PCHSig) were absent at 6 hours (hrs) post-APAP (A6, FIG. 18F). Surprisingly, at 24 hrs post-APAP, the pericentral hepatocyte expression signature returned (A24, FIG. 18F), despite histology showing persistent pericentral necrosis (A24, FIG. 18B, 18C). In particular, expression of two typically pericentrally restricted genes—Cyp2e1, responsible for metabolizing APAP, and Glu1, which assimilates ammonia into glutamine—was maintained, or returned, following pericentral injury. For example, Cyp2e1+ hepatocytes decreased from 67% (Untreated, UT) to 5% (A6), but returned back to 46% by 24 hrs with no significant change in Glu1+ hepatocytes at any time point. These results suggest the intriguing possibility of compensatory expression of pericentral genes by non-pericentral hepatocytes.

TABLE 14a
APAP vs. UT

| ID | p_val | avg_logFC | pct.1 | pct.2 | p_val_adj |
|---|---|---|---|---|---|
| Mat1a | 0 | 0.750044423 | 0.983 | 0.966 | 0 |
| Sepp1 | 0 | 0.345459872 | 1 | 1 | 0 |
| Mup3 | 0 | −0.542484173 | 0.999 | 1 | 0 |
| mt-Rnr2 | 0 | −0.545826779 | 1 | 1 | 0 |
| mt-Nd1 | 0 | −0.759986171 | 0.978 | 1 | 0 |
| mt-Nd5 | 0 | −0.779679711 | 0.942 | 0.995 | 0 |
| Selenbp2 | 0 | −0.830430695 | 0.751 | 0.937 | 0 |
| mt-Nd2 | 0 | −0.832515437 | 0.978 | 0.999 | 0 |
| Mup21 | 0 | −0.872348794 | 0.702 | 0.929 | 0 |
| mt-Cytb | 0 | −0.877546618 | 0.971 | 0.999 | 0 |
| mt-Co1 | 0 | −0.921896864 | 0.977 | 0.999 | 0 |
| Gm13775 | 0 | −1.061267629 | 0.306 | 0.761 | 0 |
| mt-Co2 | 0 | −1.077728564 | 0.157 | 0.629 | 0 |
| mt-Nd4 | 0 | −1.115678214 | 0.942 | 0.998 | 0 |
| Mup16 | 0 | −1.12499966 | 0.594 | 0.934 | 0 |
| Mup15 | 0 | −1.259561807 | 0.166 | 0.617 | 0 |
| Mup5 | 0 | −1.341986627 | 0.541 | 0.839 | 0 |
| Mup18 | 0 | −1.638566323 | 0.838 | 0.997 | 0 |
| Mup11 | 0 | −1.728428444 | 0.84 | 1 | 0 |
| Mup19 | 0 | −1.785867292 | 0.916 | 1 | 0 |
| Mup6 | 0 | −1.881843585 | 0.804 | 0.992 | 0 |
| Mup9 | 0 | −1.914490248 | 0.594 | 0.976 | 0 |
| Mup17 | 0 | −2.016622219 | 0.947 | 1 | 0 |
| Cyp4a14 | 1.20E−300 | 1.281150046 | 0.714 | 0.431 | 2.97E−296 |
| mt-Co3 | 1.54E−290 | −0.958403256 | 0.198 | 0.603 | 3.82E−286 |
| Serpina1e | 1.35E−287 | −0.468116476 | 0.99 | 0.999 | 3.36E−283 |
| Gm15564 | 1.15E−262 | 1.171130141 | 0.981 | 0.956 | 2.85E−258 |
| Akr1c6 | 4.68E−230 | −0.504664681 | 0.92 | 0.971 | 1.16E−225 |
| Ankrd55 | 6.63E−225 | −1.230858809 | 0.451 | 0.696 | 1.65E−220 |
| Lars2 | 2.20E−221 | 1.06892618 | 0.997 | 0.994 | 5.46E−217 |
| Gm26924 | 1.56E−214 | 0.85076884 | 1 | 1 | 3.86E−210 |
| Mup4 | 8.62E−211 | −1.000765415 | 0.716 | 0.898 | 2.14E−206 |
| mt-Rnr1 | 1.37E−205 | −0.353829755 | 1 | 1 | 3.40E−201 |
| C3 | 9.78E−200 | 0.350190783 | 0.997 | 0.99 | 2.43E−195 |
| Fabp1 | 3.44E−187 | −0.30775699 | 0.992 | 1 | 8.54E−183 |
| Hspa5 | 2.31E−180 | 0.404849756 | 0.971 | 0.944 | 5.73E−176 |
| Cp | 9.33E−179 | 0.375775919 | 0.965 | 0.943 | 2.32E−174 |
| Elovl3 | 5.11E−177 | −0.522135473 | 0.65 | 0.827 | 1.27E−172 |
| Gsta3 | 4.62E−168 | −0.404911812 | 0.958 | 0.956 | 1.15E−163 |
| Mup7 | 2.96E−167 | −0.601531682 | 0.937 | 0.998 | 7.35E−163 |
| n-R5-8s1 | 8.50E−167 | 0.696528218 | 0.268 | 0.031 | 2.11E−162 |
| Nudt7 | 3.51E−163 | −0.6192372 | 0.691 | 0.83 | 8.71E−159 |
| Serpina1a | 1.94E−157 | −0.33473551 | 0.96 | 0.991 | 4.81E−153 |
| Uqcrq | 7.40E−157 | −0.410137125 | 0.902 | 0.951 | 1.84E−152 |
| Hnf4a | 6.16E−155 | 0.453812022 | 0.876 | 0.743 | 1.53E−150 |
| Ttpa | 9.28E−150 | 0.393179802 | 0.916 | 0.869 | 2.30E−145 |
| Car3 | 2.61E−147 | −0.376245944 | 0.956 | 0.974 | 6.47E−143 |
| Angptl3 | 1.63E−146 | 0.414020213 | 0.961 | 0.944 | 4.04E−142 |
| Serpina3m | 1.77E−141 | 0.459745121 | 0.836 | 0.722 | 4.39E−137 |
| Fga | 3.27E−140 | 0.433534845 | 0.996 | 0.997 | 8.13E−136 |
| Fn1 | 2.00E−138 | 0.473677819 | 0.912 | 0.821 | 4.95E−134 |
| Scd1 | 4.70E−138 | 0.512243564 | 0.969 | 0.901 | 1.17E−133 |
| Ces3a | 1.36E−137 | −0.317352764 | 0.93 | 0.967 | 3.38E−133 |
| Mfsd2a | 3.04E−137 | 0.448359194 | 0.413 | 0.148 | 7.55E−133 |
| Snord118 | 3.94E−137 | 0.470651684 | 0.531 | 0.248 | 9.78E−133 |
| Mup8 | 1.52E−136 | −0.726763962 | 0.13 | 0.373 | 3.77E−132 |
| Ndrg2 | 9.66E−134 | 0.28448062 | 0.956 | 0.939 | 2.40E−129 |
| Igfbp4 | 6.05E−133 | 0.31482773 | 0.919 | 0.866 | 1.50E−128 |
| Adh1 | 1.12E−132 | −0.31221001 | 0.941 | 0.96 | 2.79E−128 |
| Serpina3n | 3.83E−132 | 0.449619441 | 0.76 | 0.574 | 9.50E−128 |
| Hp | 8.34E−132 | 0.260394974 | 0.998 | 0.999 | 2.07E−127 |
| Prdx1 | 2.41E−131 | −0.349862194 | 0.878 | 0.927 | 5.99E−127 |
| Gm24601 | 3.54E−131 | 0.4961857 | 0.198 | 0.015 | 8.80E−127 |
| Gstp1 | 4.61E−130 | −0.608807368 | 0.702 | 0.822 | 1.14E−125 |
| Hsd3b7 | 3.94E−128 | −0.450130172 | 0.693 | 0.798 | 9.79E−124 |
| Grip2 | 8.48E−125 | 0.379665448 | 0.158 | 0.002 | 2.11E−120 |
| Apoc2 | 7.71E−123 | 0.510711685 | 0.851 | 0.802 | 1.91E−118 |
| Cyp4a10 | 1.33E−120 | 0.715856279 | 0.727 | 0.657 | 3.31E−116 |
| Acaa1b | 1.79E−119 | −0.315795941 | 0.883 | 0.969 | 4.44E−115 |
| Fgb | 2.98E−118 | 0.390519359 | 0.999 | 1 | 7.40E−114 |
| Mup20 | 2.74E−117 | −0.372499315 | 0.992 | 1 | 6.81E−113 |
| Mup13 | 3.18E−117 | −0.624965563 | 0.351 | 0.546 | 7.90E−113 |
| Aldob | 1.56E−114 | 0.257594907 | 0.997 | 0.996 | 3.87E−110 |
| Lpin2 | 1.59E−114 | 0.398387997 | 0.79 | 0.597 | 3.95E−110 |
| Rsp14 | 1.62E−111 | −0.336423845 | 0.856 | 0.917 | 4.03E−107 |
| Sephs2 | 1.60E−110 | 0.428627671 | 0.865 | 0.761 | 3.97E−106 |
| Lrg1 | 1.71E−110 | 0.347811428 | 0.861 | 0.736 | 4.24E−106 |
| Slco1a4 | 2.27E−110 | 0.382614872 | 0.484 | 0.238 | 5.64E−106 |
| Pabpc1 | 4.89E−109 | 0.373452962 | 0.75 | 0.592 | 1.21E−104 |
| Cyb5 | 5.75E−109 | −0.284450423 | 0.972 | 0.983 | 1.43E−104 |
| P4hb | 2.06E−108 | 0.258583074 | 0.958 | 0.941 | 5.13E−104 |
| 2810007J24Rik | 3.82E−107 | −0.287090402 | 0.909 | 0.943 | 9.49E−103 |
| Apoa4 | 1.21E−105 | 0.598398872 | 0.574 | 0.339 | 3.01E−101 |
| Dhcr24 | 1.89E−105 | 0.362875988 | 0.883 | 0.783 | 4.69E−101 |
| Grn | 1.70E−104 | 0.369164433 | 0.62 | 0.396 | 4.21E−100 |
| Hsd3b5 | 2.16E−104 | −0.471194244 | 0.367 | 0.587 | 5.37E−100 |
| Mup12 | 3.61E−104 | −0.371327569 | 0.665 | 0.86 | 8.97E−100 |
| St3gal5 | 4.68E−104 | 0.387569967 | 0.695 | 0.49 | 1.16E−99 |
| Sparcl1 | 7.00E−103 | −0.253616986 | 0 | 0.136 | 1.74E−98 |
| Wbp1l | 1.55E−102 | 0.366182386 | 0.768 | 0.606 | 3.84E−98 |
| Hrg | 7.10E−102 | 0.321041129 | 0.88 | 0.831 | 1.76E−97 |
| Sult1a1 | 1.38E−97 | 0.365154041 | 0.799 | 0.707 | 3.42E−93 |
| Fgg | 1.52E−96 | 0.339113048 | 0.997 | 0.991 | 3.78E−92 |
| Cox4i1 | 2.61E−94 | −0.262749367 | 0.941 | 0.958 | 6.48E−90 |
| Gyk | 2.78E−94 | 0.314409774 | 0.583 | 0.35 | 6.90E−90 |
| Oaz1 | 5.51E−93 | −0.394430119 | 0.651 | 0.75 | 1.37E−88 |
| Eef2 | 2.12E−92 | 0.291153401 | 0.928 | 0.873 | 5.27E−88 |
| Tmem205 | 1.10E−91 | −0.295263774 | 0.877 | 0.934 | 2.74E−87 |
| Man2a1 | 2.30E−91 | 0.340428532 | 0.742 | 0.583 | 5.70E−87 |
| Itih3 | 2.87E−91 | 0.330302128 | 0.937 | 0.89 | 7.12E−87 |
| Itih4 | 2.97E−91 | 0.282959368 | 0.97 | 0.948 | 7.36E−87 |
| Gclc | 6.76E−90 | 0.670994545 | 0.839 | 0.718 | 1.68E−85 |
| Eif4g2 | 1.91E−89 | 0.340677509 | 0.824 | 0.678 | 4.75E−85 |
| Il1r1 | 7.47E−88 | 0.363254586 | 0.39 | 0.167 | 1.85E−83 |
| Tm4sf4 | 1.42E−87 | 0.275958137 | 0.493 | 0.256 | 3.54E−83 |
| Chchd10 | 8.26E−87 | 0.383325007 | 0.925 | 0.944 | 2.05E−82 |
| Tmed5 | 4.26E−86 | 0.302739481 | 0.689 | 0.492 | 1.06E−81 |
| Retsat | 4.94E−86 | 0.412599616 | 0.87 | 0.83 | 1.23E−81 |
| Lpgat1 | 6.97E−85 | 0.353866188 | 0.693 | 0.502 | 1.73E−80 |
| Serpina3c | 4.51E−84 | −0.425256202 | 0.208 | 0.385 | 1.12E−79 |
| Ugt2b1 | 1.86E−83 | −0.30063164 | 0.817 | 0.871 | 4.62E−79 |
| Ddx21 | 1.16E−81 | 0.257603416 | 0.447 | 0.22 | 2.87E−77 |
| Trp53inp1 | 6.93E−81 | 0.397717854 | 0.526 | 0.308 | 1.72E−76 |
| Rps2 | 3.51E−80 | −0.379370911 | 0.637 | 0.757 | 8.73E−76 |
| Myh9 | 5.00E−80 | 0.450738112 | 0.531 | 0.318 | 1.24E−75 |
| Itih2 | 7.80E−80 | 0.268155336 | 0.942 | 0.884 | 1.94E−75 |
| Gm23935 | 9.33E−80 | 0.570137239 | 0.954 | 0.925 | 2.32E−75 |
| Bach1 | 1.69E−79 | 0.261285488 | 0.356 | 0.151 | 4.19E−75 |
| Asl | 5.93E−79 | 0.337397732 | 0.859 | 0.787 | 1.47E−74 |
| Agt | 1.51E−78 | 0.300654386 | 0.859 | 0.817 | 3.75E−74 |
| Slc4a4 | 3.95E−78 | 0.282733417 | 0.558 | 0.336 | 9.81E−74 |
| Slc7a2 | 5.73E−78 | 0.333442671 | 0.829 | 0.735 | 1.42E−73 |
| Inmt | 1.03E−77 | −0.366227618 | 0.871 | 0.884 | 2.55E−73 |
| Fmo5 | 4.81E−77 | 0.254462826 | 0.914 | 0.837 | 1.19E−72 |
| Aldh9a1 | 9.61E−75 | 0.271493861 | 0.836 | 0.763 | 2.39E−70 |
| Dnajc3 | 2.25E−74 | 0.270821541 | 0.86 | 0.772 | 5.60E−70 |
| Csde1 | 4.00E−74 | 0.273863255 | 0.749 | 0.577 | 9.94E−70 |
| Agpat6 | 1.55E−73 | 0.27062679 | 0.695 | 0.517 | 3.86E−69 |
| Cox6c | 1.98E−73 | −0.343178548 | 0.808 | 0.884 | 4.92E−69 |
| Hmgcs2 | 7.75E−73 | 0.310476909 | 0.974 | 0.986 | 1.92E−68 |
| Ndufb11 | 7.98E−73 | −0.352187855 | 0.637 | 0.735 | 1.98E−68 |
| Cdo1 | 9.77E−73 | 0.266520006 | 0.964 | 0.96 | 2.43E−68 |
| Cyp7b1 | 1.18E−72 | −0.351246722 | 0.654 | 0.757 | 2.92E−68 |
| Cyp1a2 | 2.21E−72 | −0.303458132 | 0.621 | 0.794 | 5.49E−68 |
| Epb4.1 | 3.08E−72 | 0.256691619 | 0.527 | 0.304 | 7.65E−68 |
| Sh3glb1 | 9.12E−72 | 0.252065411 | 0.47 | 0.258 | 2.27E−67 |
| C8g | 9.19E−72 | −0.258246671 | 0.859 | 0.91 | 2.28E−67 |
| Rdx | 1.14E−71 | 0.282860025 | 0.775 | 0.63 | 2.82E−67 |
| Eif5 | 1.43E−71 | 0.259766522 | 0.84 | 0.74 | 3.55E−67 |
| Iigp1 | 1.79E−71 | 0.34482133 | 0.851 | 0.759 | 4.45E−67 |
| Arhgap5 | 3.14E−71 | 0.287540755 | 0.647 | 0.454 | 7.79E−67 |
| D17Wsu92e | 3.82E−71 | 0.280932562 | 0.736 | 0.588 | 9.48E−67 |
| Atp2a2 | 5.01E−71 | 0.303121987 | 0.666 | 0.495 | 1.24E−66 |
| Uroc1 | 1.56E−70 | 0.313479852 | 0.74 | 0.615 | 3.86E−66 |
| Cpt1a | 2.48E−70 | 0.285036071 | 0.775 | 0.651 | 6.15E−66 |
| Slc39a14 | 2.80E−70 | 0.355950632 | 0.552 | 0.343 | 6.96E−66 |
| Pigr | 3.30E−70 | 0.274660157 | 0.952 | 0.947 | 8.21E−66 |
| Lifr | 5.26E−70 | −0.353193545 | 0.608 | 0.694 | 1.31E−65 |
| Hamp | 1.12E−69 | −0.274177459 | 0.718 | 0.884 | 2.79E−65 |
| Etf1 | 1.81E−69 | 0.275594116 | 0.61 | 0.409 | 4.48E−65 |
| Tnfrsf12a | 2.18E−69 | 0.258809369 | 0.193 | 0.052 | 5.42E−65 |
| Abcb4 | 5.90E−69 | 0.275923839 | 0.78 | 0.675 | 1.46E−64 |
| Ranbp2 | 2.49E−68 | 0.256706494 | 0.555 | 0.338 | 6.17E−64 |
| Mbnl1 | 3.62E−68 | 0.250989808 | 0.511 | 0.297 | 9.00E−64 |
| Zfp91 | 1.31E−67 | 0.261914675 | 0.666 | 0.492 | 3.26E−63 |
| Rora | 1.63E−67 | 0.285339115 | 0.547 | 0.341 | 4.04E−63 |
| Tgoln1 | 3.36E−67 | 0.267654107 | 0.713 | 0.575 | 8.35E−63 |
| Hsd17b13 | 4.56E−67 | 0.443731877 | 0.798 | 0.74 | 1.13E−62 |
| Cfhr2 | 1.47E−66 | −0.329112182 | 0.854 | 0.868 | 3.64E−62 |
| Elovl2 | 2.70E−66 | 0.275127171 | 0.831 | 0.699 | 6.69E−62 |
| Gm2a | 4.22E−66 | 0.281407096 | 0.599 | 0.427 | 1.05E−61 |
| Dennd5b | 4.24E−66 | 0.256205947 | 0.55 | 0.334 | 1.05E−61 |
| Abca6 | 6.79E−66 | 0.26370262 | 0.679 | 0.507 | 1.69E−61 |
| Hbb-bs | 9.29E−66 | 1.33215306 | 0.125 | 0.018 | 2.31E−61 |
| Mia3 | 1.03E−65 | 0.255732645 | 0.749 | 0.61 | 2.57E−61 |
| Pten | 1.04E−65 | 0.264976186 | 0.645 | 0.458 | 2.58E−61 |
| Txnrd1 | 1.49E−65 | 0.44747429 | 0.64 | 0.441 | 3.70E−61 |
| Sdhc | 1.53E−65 | 0.260461296 | 0.738 | 0.63 | 3.81E−61 |
| Herpud1 | 2.81E−65 | 0.297286245 | 0.842 | 0.803 | 6.98E−61 |
| Ddx3x | 8.72E−64 | 0.275330763 | 0.689 | 0.513 | 2.17E−59 |
| Gfra1 | 2.53E−63 | 0.253571589 | 0.676 | 0.498 | 6.29E−59 |
| Eif1a | 4.82E−63 | 0.266737555 | 0.492 | 0.289 | 1.20E−58 |
| Fgl1 | 1.26E−62 | 0.361141465 | 0.861 | 0.784 | 3.12E−58 |
| Ell2 | 5.11E−62 | 0.257245989 | 0.609 | 0.416 | 1.27E−57 |
| Usp9x | 1.80E−61 | 0.254069319 | 0.503 | 0.299 | 4.47E−57 |
| Mup14 | 2.61E−61 | −0.524421073 | 0.469 | 0.616 | 6.49E−57 |
| Lipa | 3.53E−61 | 0.257743912 | 0.73 | 0.61 | 8.77E−57 |
| Srxn1 | 3.87E−61 | 0.563990466 | 0.369 | 0.189 | 9.60E−57 |
| Sec61a1 | 4.35E−61 | 0.264849626 | 0.642 | 0.476 | 1.08E−56 |
| Reep6 | 5.73E−61 | 0.289187421 | 0.837 | 0.794 | 1.42E−56 |
| Bche | 9.44E−61 | 0.256643093 | 0.562 | 0.369 | 2.34E−56 |
| Aplp2 | 1.24E−60 | 0.263796183 | 0.759 | 0.625 | 3.09E−56 |
| Slc40a1 | 1.54E−60 | 0.276808969 | 0.517 | 0.328 | 3.84E−56 |
| Aadac | 2.88E−59 | −0.324280061 | 0.849 | 0.86 | 7.15E−55 |
| Kif1b | 3.89E−59 | 0.263987547 | 0.629 | 0.435 | 9.66E−55 |
| Arhgef12 | 4.79E−59 | 0.278318571 | 0.597 | 0.408 | 1.19E−54 |
| Atp1a1 | 3.90E−58 | 0.287145639 | 0.623 | 0.457 | 9.68E−54 |
| Keg1 | 4.07E−58 | −0.385687903 | 0.441 | 0.545 | 1.01E−53 |
| Gm4076 | 4.99E−58 | −0.269795145 | 0.057 | 0.184 | 1.24E−53 |
| Cyp2a5 | 7.24E−58 | −0.327337224 | 0.388 | 0.555 | 1.80E−53 |
| Fam134b | 2.09E−57 | 0.298654652 | 0.589 | 0.405 | 5.19E−53 |
| Chchd2 | 3.96E−57 | −0.257495549 | 0.874 | 0.916 | 9.83E−53 |
| Creld2 | 4.34E−57 | 0.262631686 | 0.487 | 0.296 | 1.08E−52 |
| Hyou1 | 5.77E−57 | 0.365752306 | 0.561 | 0.385 | 1.43E−52 |
| Hmox1 | 9.91E−57 | 0.329838257 | 0.108 | 0.016 | 2.46E−52 |
| Ces3b | 1.20E−56 | −0.283988668 | 0.727 | 0.787 | 2.98E−52 |
| Ttc39c | 4.98E−56 | −0.318694789 | 0.573 | 0.642 | 1.24E−51 |
| Ube2r2 | 6.52E−56 | 0.260699825 | 0.655 | 0.525 | 1.62E−51 |
| Picalm | 8.44E−55 | 0.266596403 | 0.558 | 0.376 | 2.10E−50 |
| Cite | 1.40E−54 | 0.30770172 | 0.602 | 0.414 | 3.47E−50 |
| Slc25a47 | 1.66E−54 | 0.309555605 | 0.927 | 0.927 | 4.13E−50 |
| Lrrc58 | 1.38E−53 | 0.299758126 | 0.542 | 0.359 | 3.42E−49 |
| Brd2 | 1.64E−53 | 0.285550428 | 0.59 | 0.408 | 4.07E−49 |
| Gstm3 | 2.30E−53 | −0.47361305 | 0.269 | 0.414 | 5.70E−49 |
| F5 | 7.46E−53 | 0.260671616 | 0.809 | 0.728 | 1.85E−48 |
| Mt1 | 8.42E−53 | 0.890943921 | 0.701 | 0.556 | 2.09E−48 |
| Gadd45g | 9.92E−53 | −0.340515267 | 0.503 | 0.639 | 2.46E−48 |
| Apob | 1.44E−52 | 0.267840983 | 0.989 | 0.989 | 3.59E−48 |
| Hdlbp | 3.91E−52 | 0.27404334 | 0.828 | 0.713 | 9.72E−48 |
| Gjb2 | 6.01E−52 | 0.28026592 | 0.725 | 0.572 | 1.49E−47 |
| Ndufa4 | 7.60E−52 | −0.261719376 | 0.851 | 0.889 | 1.89E−47 |
| Cml1 | 7.90E−52 | −0.295319392 | 0.709 | 0.757 | 1.96E−47 |
| Sfxn1 | 5.35E−51 | 0.262434679 | 0.636 | 0.496 | 1.33E−46 |
| Thrsp | 3.45E−50 | −0.460250446 | 0.583 | 0.627 | 8.56E−46 |
| Ssr3 | 6.72E−49 | 0.252936524 | 0.732 | 0.596 | 1.67E−44 |
| mt-Atp6 | 2.06E−48 | −0.419380785 | 0.311 | 0.452 | 5.11E−44 |
| Rpl32 | 1.00E−47 | −0.30384302 | 0.688 | 0.757 | 2.49E−43 |
| Btg2 | 4.30E−45 | 0.263524699 | 0.401 | 0.241 | 1.07E−40 |
| Hspe1 | 2.75E−44 | −0.308522832 | 0.462 | 0.557 | 6.83E−40 |
| Insig1 | 1.88E−43 | 0.254088613 | 0.597 | 0.426 | 4.67E−39 |
| Mt2 | 7.29E−43 | 0.669743697 | 0.518 | 0.365 | 1.81E−38 |
| Hsd3b3 | 2.62E−41 | −0.282851137 | 0.519 | 0.605 | 6.51E−37 |
| Serpina12 | 6.83E−41 | −0.279022117 | 0.362 | 0.492 | 1.70E−36 |
| S100a9 | 7.01E−39 | 0.317582792 | 0.058 | 0.004 | 1.74E−34 |
| Prdx4 | 1.19E−38 | −0.290503348 | 0.55 | 0.607 | 2.95E−34 |
| Mup2 | 3.60E−37 | −0.446904247 | 0.285 | 0.392 | 8.95E−33 |
| Hbb-bt | 1.55E−36 | 0.448173614 | 0.052 | 0.002 | 3.86E−32 |
| Cox7a2 | 2.72E−36 | −0.251096024 | 0.722 | 0.784 | 6.76E−32 |
| Cyp2b10 | 2.41E−35 | −0.502782767 | 0.493 | 0.542 | 5.98E−31 |
| Ndufs6 | 3.36E−32 | −0.272052906 | 0.678 | 0.724 | 8.33E−28 |
| Krt18 | 5.62E−32 | 0.330676662 | 0.72 | 0.617 | 1.39E−27 |
| mmu-mir-6240 | 1.64E−30 | 0.339455801 | 0.22 | 0.115 | 4.06E−26 |
| Ndufb4 | 1.61E−29 | −0.253090085 | 0.409 | 0.49 | 3.99E−25 |
| Fos | 3.19E−28 | 0.263299308 | 0.609 | 0.51 | 7.91E−24 |
| Plin2 | 3.89E−27 | 0.371436511 | 0.799 | 0.714 | 9.66E−23 |
| Rpl36al | 8.10E−27 | −0.2702077 | 0.495 | 0.551 | 2.01E−22 |
| Mup1 | 1.57E−24 | −0.470008649 | 0.522 | 0.583 | 3.91E−20 |
| Rps15 | 4.98E−19 | −0.272193403 | 0.49 | 0.528 | 1.24E−14 |
| Ubb | 5.31E−19 | −0.264147083 | 0.394 | 0.45 | 1.32E−14 |
| Etnppl | 2.49E−17 | 0.263747104 | 0.647 | 0.606 | 6.18E−13 |
| Gm26917 | 2.71E−16 | 0.39502224 | 0.253 | 0.181 | 6.74E−12 |
| Ftl1 | 2.27E−15 | −0.309991928 | 0.367 | 0.409 | 5.64E−11 |
| Chka | 1.77E−12 | 0.28917469 | 0.312 | 0.231 | 4.40E−08 |
| mmu-mir-6236 | 0.004690575 | 0.257694673 | 0.868 | 0.837 | 1 |
| Saa2 | 0.008288556 | 0.375915223 | 0.231 | 0.222 | 1 |

TABLE 14b
PH vs UT Markers

| ID | p_val | avg_logFC | pct.1 | pct.2 | p_val_adj |
|---|---|---|---|---|---|
| Saa2 | 0 | 3.689742641 | 0.981 | 0.222 | 0 |
| Saa1 | 0 | 2.953116544 | 0.999 | 0.662 | 0 |
| Mt1 | 0 | 2.20137648 | 0.968 | 0.556 | 0 |
| Mt2 | 0 | 2.140389237 | 0.934 | 0.365 | 0 |
| Lcn2 | 0 | 1.982609697 | 0.862 | 0.07 | 0 |
| Orm2 | 0 | 1.521515479 | 0.596 | 0.09 | 0 |
| Serpina3n | 0 | 1.468184429 | 0.935 | 0.574 | 0 |
| Steap4 | 0 | 1.432404924 | 0.767 | 0.222 | 0 |
| Gm26924 | 0 | 1.269761148 | 1 | 1 | 0 |
| Apcs | 0 | 1.231432603 | 0.878 | 0.525 | 0 |
| n-R5-8s1 | 0 | 1.205558807 | 0.652 | 0.031 | 0 |
| Fgl1 | 0 | 1.13936575 | 0.973 | 0.784 | 0 |
| Hp | 0 | 1.122408731 | 1 | 0.999 | 0 |
| Orm1 | 0 | 1.121622286 | 0.996 | 0.952 | 0 |
| Lars2 | 0 | 1.06369286 | 0.999 | 0.994 | 0 |
| Itih4 | 0 | 1.059091356 | 0.997 | 0.948 | 0 |
| Lrg1 | 0 | 1.054853379 | 0.964 | 0.736 | 0 |
| Gm15564 | 0 | 1.050908678 | 0.991 | 0.956 | 0 |
| Fga | 0 | 0.99801394 | 0.998 | 0.997 | 0 |
| Hpx | 0 | 0.940144751 | 1 | 0.997 | 0 |
| Itih3 | 0 | 0.937820769 | 0.983 | 0.89 | 0 |
| Saa4 | 0 | 0.924382378 | 0.941 | 0.749 | 0 |
| Igfbp1 | 0 | 0.91788643 | 0.881 | 0.717 | 0 |
| mmu-mir-6236 | 0 | 0.830874915 | 0.969 | 0.837 | 0 |
| C3 | 0 | 0.82839829 | 0.999 | 0.99 | 0 |
| Gm23935 | 0 | 0.802263862 | 0.993 | 0.925 | 0 |
| Gm24601 | 0 | 0.792992967 | 0.498 | 0.015 | 0 |
| Serpina10 | 0 | 0.76903021 | 0.885 | 0.641 | 0 |
| Fn1 | 0 | 0.767461447 | 0.941 | 0.821 | 0 |
| Il1r1 | 0 | 0.756862098 | 0.606 | 0.167 | 0 |
| Nnmt | 0 | 0.755485266 | 0.906 | 0.716 | 0 |
| Fgg | 0 | 0.746061837 | 0.999 | 0.991 | 0 |
| Tat | 0 | 0.684122974 | 0.975 | 0.903 | 0 |
| Fgb | 0 | 0.682932942 | 1 | 1 | 0 |
| Cfh | 0 | 0.573278509 | 0.973 | 0.897 | 0 |
| Mat1a | 0 | 0.537803765 | 0.985 | 0.966 | 0 |
| Pzp | 0 | 0.475711355 | 0.993 | 0.986 | 0 |
| Ambp | 0 | 0.375515564 | 0.998 | 0.993 | 0 |
| Kng1 | 0 | 0.374458693 | 0.995 | 0.991 | 0 |
| Apoe | 0 | −0.300107411 | 1 | 1 | 0 |
| Apoa2 | 0 | −0.343336611 | 0.999 | 1 | 0 |
| Acaa1b | 0 | −0.530837085 | 0.841 | 0.969 | 0 |
| Ugt2b5 | 0 | −0.569543251 | 0.934 | 0.959 | 0 |
| Ces1d | 0 | −0.604928809 | 0.776 | 0.923 | 0 |
| Gsta3 | 0 | −0.626297488 | 0.952 | 0.956 | 0 |
| Mup20 | 0 | −0.646719137 | 0.983 | 1 | 0 |
| mt-Rnr1 | 0 | −0.656606417 | 1 | 1 | 0 |
| Cyb5 | 0 | −0.671123253 | 0.98 | 0.983 | 0 |
| mt-Rnr2 | 0 | −0.808086948 | 1 | 1 | 0 |
| mt-Atp6 | 0 | −0.874171981 | 0.096 | 0.452 | 0 |
| Mup12 | 0 | −0.892704406 | 0.548 | 0.86 | 0 |
| Mup3 | 0 | −0.904471306 | 1 | 1 | 0 |
| Mup16 | 0 | −0.969957455 | 0.672 | 0.934 | 0 |
| Mup21 | 0 | −1.050450968 | 0.552 | 0.929 | 0 |
| Cyp2b10 | 0 | −1.18690109 | 0.151 | 0.542 | 0 |
| Gm13775 | 0 | −1.201481557 | 0.193 | 0.761 | 0 |
| mt-Nd2 | 0 | −1.211183598 | 0.975 | 0.999 | 0 |
| Mup15 | 0 | −1.224451421 | 0.175 | 0.617 | 0 |
| mt-Co3 | 0 | −1.227561419 | 0.094 | 0.603 | 0 |
| mt-Nd1 | 0 | −1.258161993 | 0.985 | 1 | 0 |
| Mup11 | 0 | −1.280114586 | 0.964 | 1 | 0 |
| mt-Nd5 | 0 | −1.306454077 | 0.917 | 0.995 | 0 |
| mt-Co2 | 0 | −1.37598008 | 0.063 | 0.629 | 0 |
| mt-Cytb | 0 | −1.393705562 | 0.967 | 0.999 | 0 |
| mt-Co1 | 0 | −1.443944016 | 0.959 | 0.999 | 0 |
| Ankrd55 | 0 | −1.559084018 | 0.273 | 0.696 | 0 |
| Mup5 | 0 | −1.635008876 | 0.359 | 0.839 | 0 |
| mt-Nd4 | 0 | −1.736336239 | 0.926 | 0.998 | 0 |
| Mup4 | 0 | −1.869924265 | 0.28 | 0.898 | 0 |
| Mup17 | 0 | −2.409962327 | 0.816 | 1 | 0 |
| Mup9 | 0 | −2.4152335 | 0.333 | 0.976 | 0 |
| Mup6 | 0 | −2.505776694 | 0.589 | 0.992 | 0 |
| Mup18 | 0 | −2.546220887 | 0.512 | 0.997 | 0 |
| Mup19 | 0 | −3.190512309 | 0.648 | 1 | 0 |
| Rbp4 | 5.65E−307 | −0.323113791 | 0.995 | 0.999 | 1.40E−302 |
| Car3 | 1.27E−295 | −0.584176786 | 0.908 | 0.974 | 3.16E−291 |
| Lpin1 | 1.18E−294 | 0.866565964 | 0.66 | 0.31 | 2.93E−290 |
| Mup13 | 3.27E−293 | −0.819687605 | 0.201 | 0.546 | 8.12E−289 |
| Clu | 3.19E−291 | 0.456701196 | 0.986 | 0.958 | 7.92E−287 |
| Fmo5 | 3.56E−281 | 0.516820181 | 0.936 | 0.837 | 8.84E−277 |
| Scd1 | 5.92E−279 | 0.79323843 | 0.966 | 0.901 | 1.47E−274 |
| Serpina3m | 5.01E−275 | 0.584599049 | 0.873 | 0.722 | 1.24E−270 |
| Tacc2 | 5.43E−271 | 0.548585735 | 0.428 | 0.071 | 1.35E−266 |
| Slc39a14 | 1.76E−266 | 0.617728658 | 0.675 | 0.343 | 4.37E−262 |
| Sephs2 | 1.85E−265 | 0.525890516 | 0.896 | 0.761 | 4.60E−261 |
| Mup8 | 4.61E−265 | −0.812904236 | 0.065 | 0.373 | 1.15E−260 |
| Rnase4 | 1.95E−264 | 0.695293911 | 0.976 | 0.921 | 4.85E−260 |
| Serpina3c | 2.75E−263 | −0.576033114 | 0.076 | 0.385 | 6.82E−259 |
| Cxcl1 | 4.03E−262 | 0.809516105 | 0.645 | 0.314 | 1.00E−257 |
| Qsox1 | 4.32E−257 | 0.54389261 | 0.887 | 0.707 | 1.07E−252 |
| Egfr | 1.70E−249 | 0.525141895 | 0.938 | 0.809 | 4.21E−245 |
| Cp | 3.84E−248 | 0.462891672 | 0.984 | 0.943 | 9.54E−244 |
| Akr1c6 | 2.09E−247 | −0.457244965 | 0.965 | 0.971 | 5.19E−243 |
| Mup10 | 2.25E−246 | 0.340599862 | 0.899 | 0.692 | 5.58E−242 |
| Cyp2c29 | 1.52E−245 | −0.719965927 | 0.772 | 0.93 | 3.77E−241 |
| Prg4 | 5.38E−245 | 0.577764422 | 0.549 | 0.188 | 1.34E−240 |
| Cox4i1 | 4.28E−244 | −0.421671698 | 0.933 | 0.958 | 1.06E−239 |
| Cyp2c54 | 6.04E−243 | −0.645057569 | 0.471 | 0.759 | 1.50E−238 |
| Cyp3a11 | 2.52E−239 | −0.296384714 | 0.999 | 1 | 6.25E−235 |
| Plg | 6.49E−234 | 0.359061279 | 0.987 | 0.961 | 1.61E−229 |
| Ly6e | 9.54E−234 | 0.714707911 | 0.683 | 0.386 | 2.37E−229 |
| Hspb8 | 3.35E−233 | 0.531530338 | 0.812 | 0.597 | 8.32E−229 |
| Mup2 | 7.36E−233 | −0.693969951 | 0.098 | 0.392 | 1.83E−228 |
| Sod1 | 7.22E−231 | −0.322303248 | 0.983 | 0.983 | 1.79E−226 |
| Vtn | 9.66E−226 | 0.319614974 | 0.994 | 0.983 | 2.40E−221 |
| Hnf4a | 4.11E−221 | 0.49323747 | 0.875 | 0.743 | 1.02E−216 |
| Fabp1 | 1.66E−220 | −0.337243781 | 0.997 | 1 | 4.11E−216 |
| Igfbp2 | 2.43E−220 | −0.509220055 | 0.742 | 0.885 | 6.03E−216 |
| Elovl3 | 1.67E−219 | −0.552813954 | 0.619 | 0.827 | 4.14E−215 |
| Pck1 | 2.26E−213 | 0.356838712 | 0.982 | 0.987 | 5.60E−209 |
| Apoc4 | 4.63E−211 | −0.333934616 | 0.985 | 0.98 | 1.15E−206 |
| Mgst1 | 9.64E−211 | −0.329076093 | 0.999 | 0.997 | 2.39E−206 |
| Cyp2c50 | 1.98E−209 | −0.605557967 | 0.707 | 0.888 | 4.92E−205 |
| Cyp2c37 | 2.30E−201 | −0.645818031 | 0.292 | 0.608 | 5.72E−197 |
| Gjb2 | 3.64E−200 | 0.499499448 | 0.789 | 0.572 | 9.05E−196 |
| Tifa | 2.20E−197 | 0.319491862 | 0.306 | 0.036 | 5.46E−193 |
| Btg2 | 7.63E−194 | 0.556244998 | 0.546 | 0.241 | 1.89E−189 |
| Fkbp5 | 9.37E−192 | 0.393979234 | 0.427 | 0.117 | 2.33E−187 |
| Ugt2b36 | 1.11E−191 | −0.379582842 | 0.908 | 0.93 | 2.77E−187 |
| C4bp | 4.37E−189 | 0.413371192 | 0.921 | 0.788 | 1.09E−184 |
| Cyp3a25 | 9.86E−189 | −0.396588983 | 0.861 | 0.919 | 2.45E−184 |
| Acox1 | 8.58E−186 | 0.351743194 | 0.97 | 0.966 | 2.13E−181 |
| Actb | 2.50E−182 | 0.376789439 | 0.956 | 0.886 | 6.22E−178 |
| Cox6c | 2.51E−182 | −0.497413871 | 0.794 | 0.884 | 6.22E−178 |
| Ahcy | 1.37E−181 | −0.430442246 | 0.835 | 0.916 | 3.40E−177 |
| Slc38a2 | 2.21E−180 | 0.526051309 | 0.671 | 0.413 | 5.50E−176 |
| mt-Nd6 | 1.13E−174 | −0.578855337 | 0.303 | 0.577 | 2.81E−170 |
| Igfbp4 | 2.03E−174 | 0.3361593 | 0.932 | 0.866 | 5.03E−170 |
| Apob | 1.78E−173 | 0.382325227 | 0.989 | 0.989 | 4.41E−169 |
| Slc41a2 | 2.85E−173 | 0.382165778 | 0.423 | 0.134 | 7.07E−169 |
| B3galt1 | 1.82E−172 | 0.521682374 | 0.446 | 0.163 | 4.53E−168 |
| Glud1 | 3.14E−171 | 0.334724449 | 0.964 | 0.905 | 7.80E−167 |
| P4hb | 9.55E−171 | 0.304018972 | 0.983 | 0.941 | 2.37E−166 |
| Slc25a25 | 4.57E−169 | 0.429973659 | 0.849 | 0.704 | 1.14E−164 |
| Gm4076 | 2.11E−168 | −0.343049018 | 0.006 | 0.184 | 5.25E−164 |
| Cyp8b1 | 3.50E−167 | −0.481824636 | 0.553 | 0.745 | 8.69E−163 |
| Myh9 | 6.99E−167 | 0.49004916 | 0.601 | 0.318 | 1.74E−162 |
| Hsd3b5 | 1.80E−161 | −0.619275352 | 0.374 | 0.587 | 4.48E−157 |
| Sdc4 | 6.03E−161 | 0.338637436 | 0.938 | 0.867 | 1.50E−156 |
| Stat3 | 8.58E−161 | 0.435626527 | 0.596 | 0.318 | 2.13E−156 |
| Uqcrq | 1.69E−160 | −0.375118557 | 0.933 | 0.951 | 4.20E−156 |
| Lrp1 | 2.72E−160 | 0.552561179 | 0.792 | 0.591 | 6.77E−156 |
| Cyp2a5 | 1.64E−158 | −0.806866679 | 0.292 | 0.555 | 4.07E−154 |
| Rps27l | 2.91E−157 | −0.450411941 | 0.748 | 0.849 | 7.24E−153 |
| Faml34b | 1.52E−155 | 0.505513689 | 0.637 | 0.405 | 3.76E−151 |
| Snx10 | 1.02E−154 | 0.374844995 | 0.464 | 0.188 | 2.53E−150 |
| Tmem176a | 1.64E−154 | 0.468074534 | 0.671 | 0.429 | 4.06E−150 |
| Arhgef26 | 6.46E−154 | 0.379910814 | 0.483 | 0.203 | 1.60E−149 |
| Aldh1l1 | 7.31E−154 | 0.344751078 | 0.962 | 0.904 | 1.81E−149 |
| Rsp14 | 1.24E−153 | −0.359378616 | 0.859 | 0.917 | 3.09E−149 |
| Slc10a2 | 1.69E−153 | 0.252457327 | 0.285 | 0.05 | 4.20E−149 |
| Angptl3 | 2.80E−152 | −0.315450205 | 0.944 | 0.944 | 6.95E−148 |
| 2810007J24Rik | 5.24E−152 | −0.351230039 | 0.876 | 0.943 | 1.30E−147 |
| Dhcr24 | 4.52E−144 | 0.459081587 | 0.912 | 0.783 | 1.12E−139 |
| Inmt | 2.84E−143 | −0.462286253 | 0.842 | 0.884 | 7.06E−139 |
| Tmem176b | 9.96E−143 | 0.449221175 | 0.803 | 0.632 | 2.47E−138 |
| Ifitm3 | 5.65E−142 | 0.361933739 | 0.91 | 0.812 | 1.40E−137 |
| Hc | 1.34E−141 | 0.372595616 | 0.949 | 0.903 | 3.33E−137 |
| Aass | 2.32E−141 | 0.442569419 | 0.837 | 0.716 | 5.75E−137 |
| Ctsb | 3.68E−141 | 0.380577928 | 0.9 | 0.781 | 9.13E−137 |
| Cpb2 | 6.91E−141 | 0.349207497 | 0.927 | 0.813 | 1.72E−136 |
| Hyou1 | 8.18E−141 | 0.42831956 | 0.638 | 0.385 | 2.03E−136 |
| Bcl3 | 1.44E−140 | 0.254384767 | 0.308 | 0.074 | 3.56E−136 |
| Rcan1 | 9.55E−137 | 0.297888563 | 0.317 | 0.085 | 2.37E−132 |
| St3gal5 | 1.41E−135 | 0.486994688 | 0.701 | 0.49 | 3.51E−131 |
| Pfkfb3 | 3.04E−135 | 0.259894095 | 0.243 | 0.039 | 7.56E−131 |
| Hrsp12 | 2.39E−134 | −0.35371508 | 0.889 | 0.939 | 5.93E−130 |
| Cox7b | 2.39E−133 | −0.426009228 | 0.809 | 0.878 | 5.93E−129 |
| Sparcl1 | 2.50E−133 | −0.253616986 | 0 | 0.136 | 6.22E−129 |
| Cldn14 | 3.85E−132 | 0.28211242 | 0.298 | 0.076 | 9.56E−128 |
| Errfi1 | 4.39E−131 | 0.31039351 | 0.953 | 0.911 | 1.09E−126 |
| Apoa4 | 1.99E−129 | 0.679443346 | 0.572 | 0.339 | 4.93E−125 |
| Rhob | 5.41E−128 | 0.25813998 | 0.287 | 0.069 | 1.34E−123 |
| Creg1 | 8.19E−128 | −0.315552444 | 0.894 | 0.926 | 2.03E−123 |
| Slc7a2 | 2.08E−124 | 0.399579476 | 0.823 | 0.735 | 5.17E−120 |
| C4b | 2.43E−124 | 0.391765742 | 0.937 | 0.883 | 6.04E−120 |
| Itih2 | 3.35E−124 | 0.299717266 | 0.946 | 0.884 | 8.32E−120 |
| Abca1 | 4.28E−124 | 0.403462344 | 0.621 | 0.377 | 1.06E−119 |
| Hsp90ab1 | 1.07E−123 | 0.279914616 | 0.967 | 0.922 | 2.66E−119 |
| Lbp | 6.64E−123 | 0.38653516 | 0.577 | 0.333 | 1.65E−118 |
| Calr | 2.26E−122 | 0.28388138 | 0.937 | 0.885 | 5.61E−118 |
| Pon1 | 2.91E−120 | −0.337990401 | 0.868 | 0.906 | 7.23E−116 |
| Etnk2 | 1.29E−119 | 0.372806041 | 0.793 | 0.655 | 3.21E−115 |
| Mtss1 | 1.02E−118 | 0.372989855 | 0.657 | 0.454 | 2.54E−114 |
| Ndufb6 | 8.09E−118 | −0.413332747 | 0.651 | 0.759 | 2.01E−113 |
| Gm20594 | 1.01E−117 | −0.541666642 | 0.102 | 0.294 | 2.50E−113 |
| Serping1 | 1.10E−117 | 0.304093562 | 0.933 | 0.87 | 2.74E−113 |
| Slc25a15 | 4.49E−117 | 0.330021491 | 0.856 | 0.738 | 1.12E−112 |
| Lpgat1 | 1.65E−116 | 0.384155769 | 0.7 | 0.502 | 4.10E−112 |
| Grn | 2.86E−115 | 0.350498754 | 0.627 | 0.396 | 7.10E−111 |
| Plin2 | 5.72E−115 | 0.400511805 | 0.807 | 0.714 | 1.42E−110 |
| H3f3b | 8.65E−115 | 0.440105486 | 0.62 | 0.419 | 2.15E−110 |
| Ech1 | 2.35E−113 | −0.335883409 | 0.78 | 0.856 | 5.83E−109 |
| Gm26917 | 2.94E−113 | 0.46154986 | 0.413 | 0.181 | 7.31E−109 |
| Zfp683 | 5.62E−113 | −0.421689112 | 0 | 0.116 | 1.40E−108 |
| Rbp1 | 2.51E−111 | 0.25848775 | 0.266 | 0.069 | 6.24E−107 |
| Rnf125 | 4.39E−111 | −0.401552322 | 0.295 | 0.493 | 1.09E−106 |
| Bhlhe40 | 5.21E−111 | 0.31908752 | 0.493 | 0.251 | 1.29E−106 |
| Mfsd2a | 1.96E−110 | 0.296148518 | 0.376 | 0.148 | 4.86E−106 |
| Ndufb11 | 6.07E−110 | −0.41040464 | 0.611 | 0.735 | 1.51E−105 |
| Cyp2d9 | 4.91E−108 | −0.27539583 | 0.94 | 0.959 | 1.22E−103 |
| Rps2 | 1.52E−107 | −0.4101328 | 0.613 | 0.757 | 3.78E−103 |
| Comt | 1.91E−107 | 0.259910468 | 0.965 | 0.931 | 4.74E−103 |
| Atf3 | 2.53E−107 | 0.402014064 | 0.399 | 0.179 | 6.29E−103 |
| Bach1 | 1.21E−106 | 0.286794151 | 0.372 | 0.151 | 2.99E−102 |
| Aadac | 1.56E−106 | −0.407054513 | 0.84 | 0.86 | 3.87E−102 |
| C8a | 2.53E−106 | 0.300098611 | 0.933 | 0.887 | 6.29E−102 |
| Cps1 | 5.25E−106 | 0.254838343 | 0.978 | 0.971 | 1.30E−101 |
| Gfra1 | 9.01E−106 | 0.342777741 | 0.696 | 0.498 | 2.24E−101 |
| Eef1a1 | 1.02E−105 | 0.27081899 | 0.913 | 0.85 | 2.53E−101 |
| Chchd2 | 3.57E−105 | −0.334788452 | 0.875 | 0.916 | 8.85E−101 |
| Plk3 | 1.17E−104 | 0.3008626 | 0.458 | 0.226 | 2.90E−100 |
| Cfi | 1.89E−104 | 0.259039083 | 0.953 | 0.905 | 4.69E−100 |
| Shb | 1.40E−103 | 0.277770125 | 0.432 | 0.204 | 3.47E−99 |
| Mpeg1 | 3.00E−103 | 0.287855502 | 0.286 | 0.089 | 7.45E−99 |
| Cfhr2 | 1.38E−102 | −0.383650774 | 0.851 | 0.868 | 3.43E−98 |
| H6pd | 7.14E−102 | 0.329467549 | 0.747 | 0.595 | 1.77E−97 |
| Retsat | 3.66E−101 | −0.353340297 | 0.69 | 0.83 | 9.09E−97 |
| Atp5f1 | 4.54E−101 | −0.336466912 | 0.845 | 0.869 | 1.13E−96 |
| Acsl1 | 5.82E−101 | −0.318662804 | 0.854 | 0.924 | 1.44E−96 |
| Itih1 | 5.89E−101 | 0.301421351 | 0.906 | 0.816 | 1.46E−96 |
| Ugt2b34 | 1.10E−100 | −0.304452716 | 0.834 | 0.874 | 2.72E−96 |
| Malat1 | 1.50E−100 | −0.401852303 | 0.613 | 0.78 | 3.72E−96 |
| Tgm2 | 7.01E−100 | 0.337876607 | 0.477 | 0.258 | 1.74E−95 |
| Ndufa4 | 1.87E−99 | −0.338157892 | 0.843 | 0.889 | 4.63E−95 |
| Saa3 | 2.83E−98 | 1.098349208 | 0.272 | 0.087 | 7.03E−94 |
| Dap | 5.60E−98 | 0.318746478 | 0.802 | 0.654 | 1.39E−93 |
| Cyp2c70 | 7.42E−98 | 0.309156286 | 0.927 | 0.834 | 1.84E−93 |
| Cdkn1a | 1.98E−97 | 0.305119488 | 0.418 | 0.195 | 4.92E−93 |
| Egr1 | 8.67E−97 | 0.418876559 | 0.627 | 0.447 | 2.15E−92 |
| Slc3a1 | 1.14E−96 | 0.32839591 | 0.401 | 0.189 | 2.84E−92 |
| Tnfrsf1a | 3.20E−96 | 0.294748954 | 0.466 | 0.244 | 7.96E−92 |
| Alas1 | 2.50E−95 | 0.322730245 | 0.857 | 0.729 | 6.20E−91 |
| Hsd3b7 | 2.77E−95 | −0.344848377 | 0.732 | 0.798 | 6.88E−91 |
| Litaf | 1.88E−93 | 0.304879493 | 0.49 | 0.278 | 4.67E−89 |
| Ifitm2 | 5.90E−93 | 0.309819544 | 0.55 | 0.347 | 1.46E−88 |
| Xbp1 | 6.66E−93 | 0.326303947 | 0.727 | 0.57 | 1.65E−88 |
| Esd | 7.38E−93 | −0.332305281 | 0.701 | 0.784 | 1.83E−88 |
| Eif4g2 | 1.69E−92 | 0.318021838 | 0.812 | 0.678 | 4.20E−88 |
| Cyp4a10 | 4.95E−92 | −0.425069794 | 0.481 | 0.657 | 1.23E−87 |
| Gpt2 | 2.14E−91 | 0.303778712 | 0.817 | 0.725 | 5.32E−87 |
| Cml2 | 5.10E−91 | 0.279615284 | 0.921 | 0.857 | 1.27E−86 |
| Cth | 1.52E−90 | −0.324114713 | 0.818 | 0.838 | 3.77E−86 |
| Trib1 | 1.01E−89 | 0.345110824 | 0.51 | 0.308 | 2.52E−85 |
| Ephx2 | 1.32E−89 | −0.279612105 | 0.886 | 0.937 | 3.27E−85 |
| Mpc2 | 4.49E−89 | −0.292946135 | 0.806 | 0.857 | 1.11E−84 |
| Sult1a1 | 4.64E−89 | 0.326135758 | 0.813 | 0.707 | 1.15E−84 |
| Rpl13a | 5.96E−89 | −0.255832076 | 0.8 | 0.868 | 1.48E−84 |
| Got1 | 1.28E−88 | 0.349895796 | 0.73 | 0.587 | 3.18E−84 |
| Gm10250 | 8.85E−88 | −0.316106405 | 0.095 | 0.251 | 2.20E−83 |
| Slc25a22 | 1.55E−86 | 0.257081972 | 0.495 | 0.282 | 3.84E−82 |
| Glul | 1.80E−86 | 0.516246513 | 0.385 | 0.18 | 4.47E−82 |
| Rapgef4 | 2.38E−85 | 0.320885046 | 0.62 | 0.436 | 5.90E−81 |
| Adck3 | 5.88E−84 | −0.332543616 | 0.695 | 0.764 | 1.46E−79 |
| Ang | 9.86E−84 | 0.394462539 | 0.811 | 0.687 | 2.45E−79 |
| Xdh | 1.19E−83 | 0.28935916 | 0.472 | 0.258 | 2.95E−79 |
| Man2a1 | 1.36E−83 | 0.31713355 | 0.734 | 0.583 | 3.38E−79 |
| Epas1 | 1.79E−83 | 0.265618306 | 0.505 | 0.297 | 4.44E−79 |
| Selenbp2 | 4.88E−83 | −0.258842945 | 0.867 | 0.937 | 1.21E−78 |
| Txnip | 5.00E−83 | 0.280540465 | 0.51 | 0.297 | 1.24E−78 |
| Cd51 | 9.74E−82 | 0.30004343 | 0.158 | 0.026 | 2.42E−77 |
| Fabp2 | 3.34E−81 | −0.375664441 | 0.473 | 0.622 | 8.30E−77 |
| Fh1 | 4.65E−81 | 0.2953128 | 0.798 | 0.648 | 1.16E−76 |
| Ugt2a3 | 1.16E−80 | −0.326140058 | 0.686 | 0.773 | 2.88E−76 |
| Gm5096 | 3.98E−80 | −0.368621118 | 0.314 | 0.468 | 9.88E−76 |
| Oaz1 | 4.28E−80 | −0.35427991 | 0.673 | 0.75 | 1.06E−75 |
| Hsd11b1 | 1.51E−79 | −0.28823804 | 0.819 | 0.875 | 3.74E−75 |
| Hrg | 5.55E−79 | 0.287679276 | 0.911 | 0.831 | 1.38E−74 |
| Atp5j | 1.74E−78 | −0.306893156 | 0.658 | 0.757 | 4.31E−74 |
| Gstm1 | 2.88E−78 | −0.312458518 | 0.91 | 0.935 | 7.16E−74 |
| Uroc1 | 9.57E−78 | 0.326783149 | 0.741 | 0.615 | 2.38E−73 |
| Ivns1abp | 9.91E−78 | 0.281892175 | 0.512 | 0.315 | 2.46E−73 |
| Hdlbp | 1.25E−77 | 0.295510036 | 0.823 | 0.713 | 3.11E−73 |
| Mvd | 1.52E−77 | −0.257890778 | 0.112 | 0.269 | 3.78E−73 |
| Clec4f | 1.69E−77 | 0.286061284 | 0.184 | 0.043 | 4.20E−73 |
| Gpcpd1 | 1.51E−76 | 0.282654586 | 0.587 | 0.398 | 3.74E−72 |
| Ndufa6 | 2.82E−75 | −0.322691385 | 0.745 | 0.798 | 7.00E−71 |
| Atp5h | 1.24E−73 | −0.414641214 | 0.575 | 0.682 | 3.08E−69 |
| Ndufb9 | 3.72E−73 | −0.256394271 | 0.841 | 0.888 | 9.23E−69 |
| Il6st | 9.58E−73 | 0.286771056 | 0.472 | 0.273 | 2.38E−68 |
| Ces1f | 1.88E−72 | −0.281138036 | 0.759 | 0.817 | 4.68E−68 |
| Clpx | 4.35E−72 | −0.31005374 | 0.733 | 0.783 | 1.08E−67 |
| Fam35a | 6.84E−72 | 0.255039805 | 0.435 | 0.246 | 1.70E−67 |
| Kmo | 2.31E−70 | 0.256834292 | 0.822 | 0.732 | 5.74E−66 |
| Fos | 2.62E−70 | 0.342537163 | 0.66 | 0.51 | 6.52E−66 |
| Hagh | 2.65E−70 | −0.26145888 | 0.835 | 0.858 | 6.58E−66 |
| Atp5j2 | 7.98E−70 | −0.286422324 | 0.796 | 0.852 | 1.98E−65 |
| Hebp1 | 1.62E−69 | −0.296503386 | 0.67 | 0.75 | 4.03E−65 |
| Ces2a | 1.70E−69 | −0.282358828 | 0.765 | 0.846 | 4.21E−65 |
| Gm9843 | 1.88E−69 | −0.34994897 | 0.155 | 0.299 | 4.66E−65 |
| Cyb5r3 | 2.44E−69 | 0.263785288 | 0.824 | 0.725 | 6.06E−65 |
| Hint2 | 3.13E−69 | −0.327239077 | 0.589 | 0.676 | 7.76E−65 |
| Mup7 | 5.98E−69 | −0.564270613 | 0.974 | 0.998 | 1.48E−64 |
| Cpt1a | 3.17E−66 | 0.268110758 | 0.758 | 0.651 | 7.88E−62 |
| Cyp3a13 | 9.97E−65 | 0.262155243 | 0.717 | 0.569 | 2.48E−60 |
| Mup1 | 2.02E−64 | −0.63630453 | 0.454 | 0.583 | 5.02E−60 |
| Upp2 | 3.37E−64 | 0.284431808 | 0.761 | 0.673 | 8.36E−60 |
| Aqp8 | 4.21E−64 | −0.318028572 | 0.372 | 0.505 | 1.05E−59 |
| Uqcr11 | 5.01E−64 | −0.256632134 | 0.833 | 0.879 | 1.24E−59 |
| Fasn | 1.22E−62 | 0.272244147 | 0.527 | 0.345 | 3.03E−58 |
| Wbp1l | 2.18E−62 | 0.251700028 | 0.732 | 0.606 | 5.40E−58 |
| Mtusl | 5.66E−62 | 0.30024906 | 0.596 | 0.42 | 1.40E−57 |
| Jun | 5.97E−62 | 0.293637263 | 0.723 | 0.593 | 1.48E−57 |
| Uqcrh | 9.05E−62 | −0.279749266 | 0.742 | 0.81 | 2.25E−57 |
| Ddx3x | 1.29E−61 | 0.264853975 | 0.655 | 0.513 | 3.21E−57 |
| Ell2 | 5.00E−61 | 0.250568745 | 0.582 | 0.416 | 1.24E−56 |
| Hsph1 | 5.93E−61 | 0.270501218 | 0.434 | 0.255 | 1.47E−56 |
| Krt18 | 1.41E−60 | 0.282094099 | 0.735 | 0.617 | 3.50E−56 |
| Atp2a2 | 1.67E−58 | 0.260679425 | 0.645 | 0.495 | 4.15E−54 |
| Fam25c | 6.81E−55 | 0.371649508 | 0.519 | 0.356 | 1.69E−50 |
| Cox5b | 5.78E−52 | −0.290064648 | 0.621 | 0.726 | 1.43E−47 |
| Ppp2r5a | 5.65E−49 | −0.281184325 | 0.506 | 0.594 | 1.40E−44 |
| Glo1 | 2.28E−48 | −0.255075136 | 0.682 | 0.749 | 5.65E−44 |
| Mug2 | 1.71E−47 | 0.260256341 | 0.813 | 0.738 | 4.24E−43 |
| 1110008F13Rik | 1.62E−46 | −0.258608944 | 0.559 | 0.648 | 4.03E−42 |
| Cyp1a2 | 1.66E−46 | −0.252439299 | 0.704 | 0.794 | 4.11E−42 |
| Prdx4 | 2.77E−43 | −0.286424739 | 0.534 | 0.607 | 6.87E−39 |
| Rpl14 | 2.85E−43 | −0.261131992 | 0.474 | 0.574 | 7.07E−39 |
| Cox7a2 | 6.39E−41 | −0.253037939 | 0.746 | 0.784 | 1.59E−36 |
| Sco2 | 4.21E−39 | −0.257919482 | 0.407 | 0.508 | 1.04E−34 |
| Rabac1 | 6.06E−39 | −0.261231844 | 0.428 | 0.524 | 1.51E−34 |
| Ndufc2 | 2.62E−38 | −0.293093496 | 0.518 | 0.579 | 6.51E−34 |
| Mup14 | 3.52E−38 | −0.4939069 | 0.539 | 0.616 | 8.73E−34 |
| Slc25a47 | 5.57E−37 | 0.255728584 | 0.908 | 0.927 | 1.38E−32 |
| Uqcrb | 1.37E−33 | −0.261145805 | 0.583 | 0.641 | 3.41E−29 |
| Nr1i3 | 2.64E−32 | −0.254276065 | 0.277 | 0.377 | 6.54E−28 |
| Thrsp | 8.05E−31 | 0.301660902 | 0.712 | 0.627 | 2.00E−26 |
| Ftl1 | 1.01E−21 | −0.326164787 | 0.35 | 0.409 | 2.50E−17 |
| Sds | 3.64E−15 | 0.384727442 | 0.641 | 0.67 | 9.04E−11 |

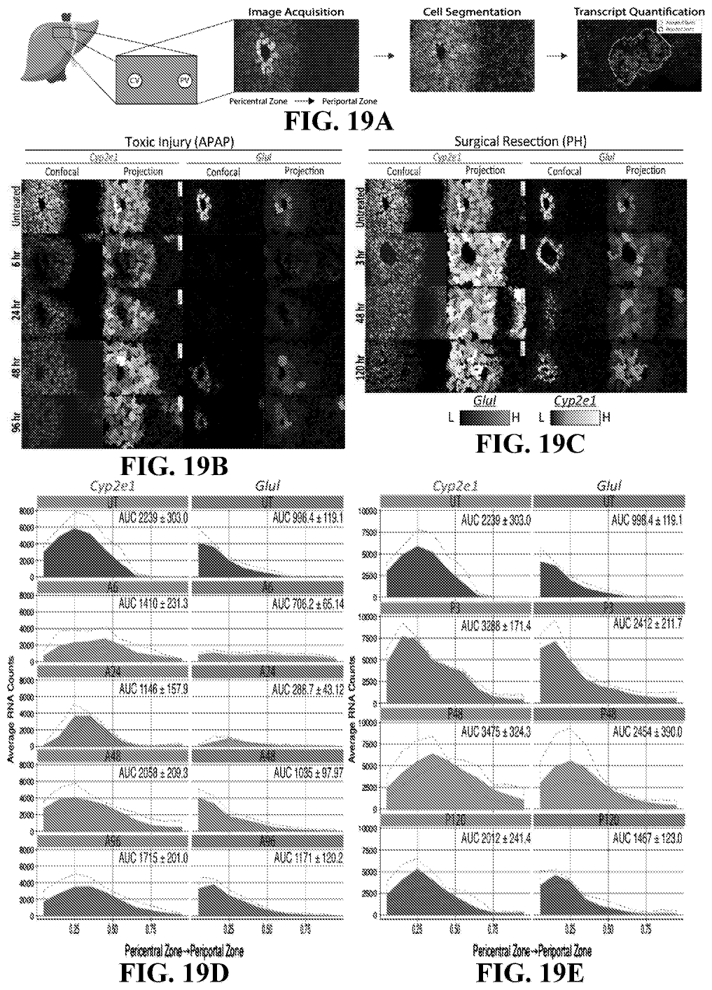

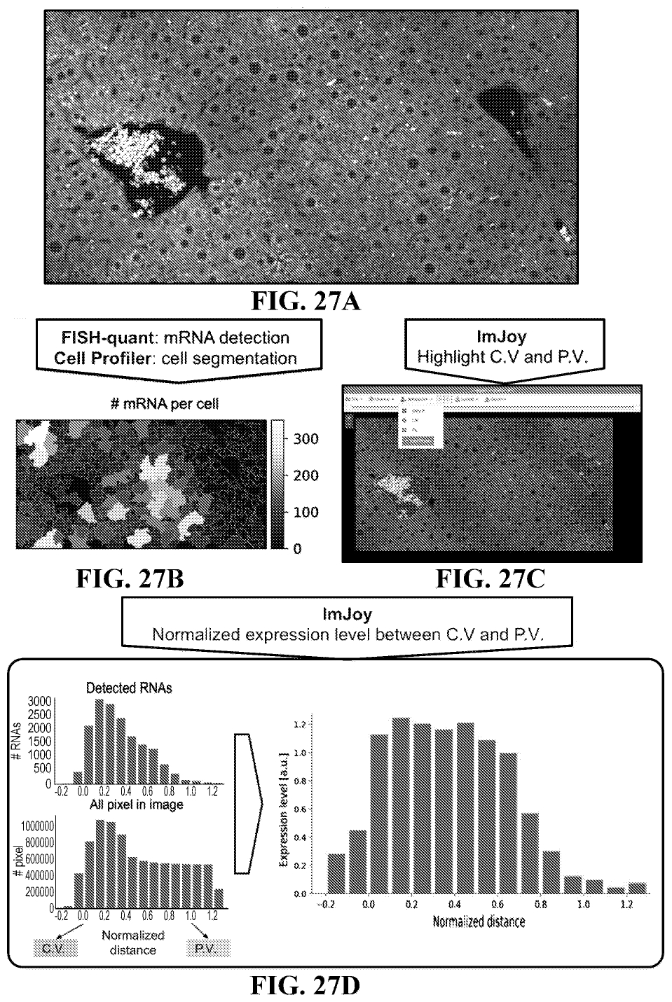

To validate these findings in a spatiotemporal context, Applicants queried the distribution of the pericentral markers Cyp2e1 and Glu1 using smFISH analyses (FIG. 19A, 19E; FIG. 27A-27D). Cyp2e1 extended further into the lobular midzone following APAP exposure, with pericentral necrosis at A6 and A24 (FIG. 19B). Expression then normalized at A48, following the cell proliferative response. Glu1 expression is normally restricted to a single layer of cells surrounding the central vein²⁵, which underwent necrosis following APAP overdose (FIG. 19B). Surprisingly, Glu1 was now expressed at low levels across the entire liver lobule indicating an effort to maintain Glu1 expression, but (A6, A24, FIG. 19B). Glu1 expression patterns returns to normal by A48 (FIG. 19B). These findings reveal compensatory expression of pericentral genes through adaptive reprogramming of midzonal hepatocytes after pericentral toxic injury.

In contrast to APAP, PH does not produce zone-dependent injury but a massive loss of ˜70% of liver cell mass (compared to ˜10% total cell loss after APAP exposure), imposing extreme functional demand on the remaining hepatocytes. Functional compensation was also observed after PH, evident from a dramatic increase in Glu1+ hepatoctyes (FIG. 18G) from 18% (Control) to 60% (P3). This is further supported by the observation that Cyp2e1+ hepatocytes only decrease by 18% (67% to 49%) at P3 (FIG. 18G). smFISH analysis confirmed increased expression zones and total expression levels for both Cyp2e1 and Glu1 in PH (FIG. 19C). In addition, Applicants observed functional compensatory up-regulation of the periportal marker Arg1 suggesting that adaptive reprogramming of hepatocytes is not exclusive to pericentral genes (FIG. 27D). These results highlight that zonal transcriptional compensation is independent of the form of liver injury occurring after both zone-specific injury and also after massive cellular loss.

### Acute Liver Injury Causes Both Injury-Specific and Non-Specific Responses

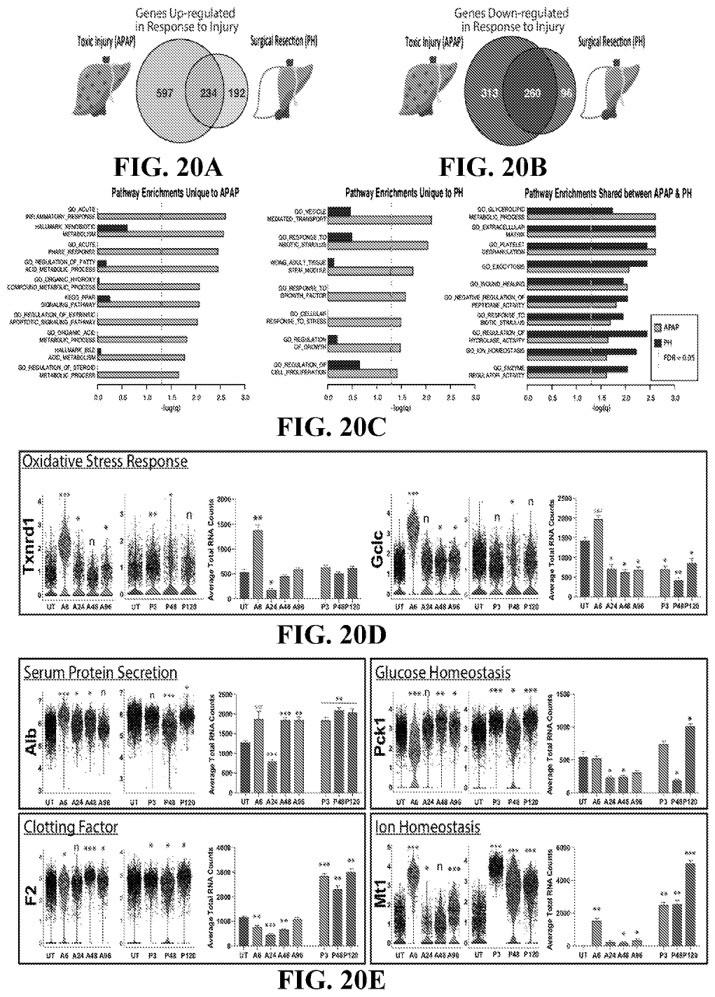

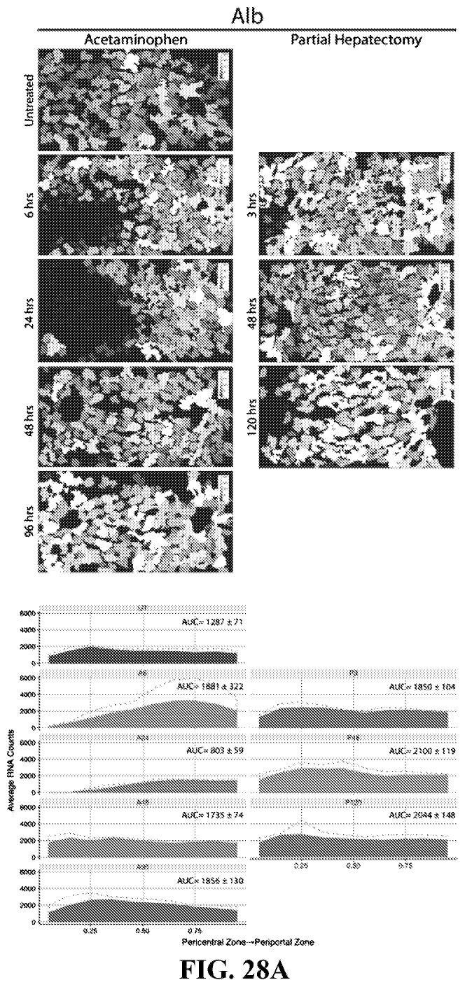

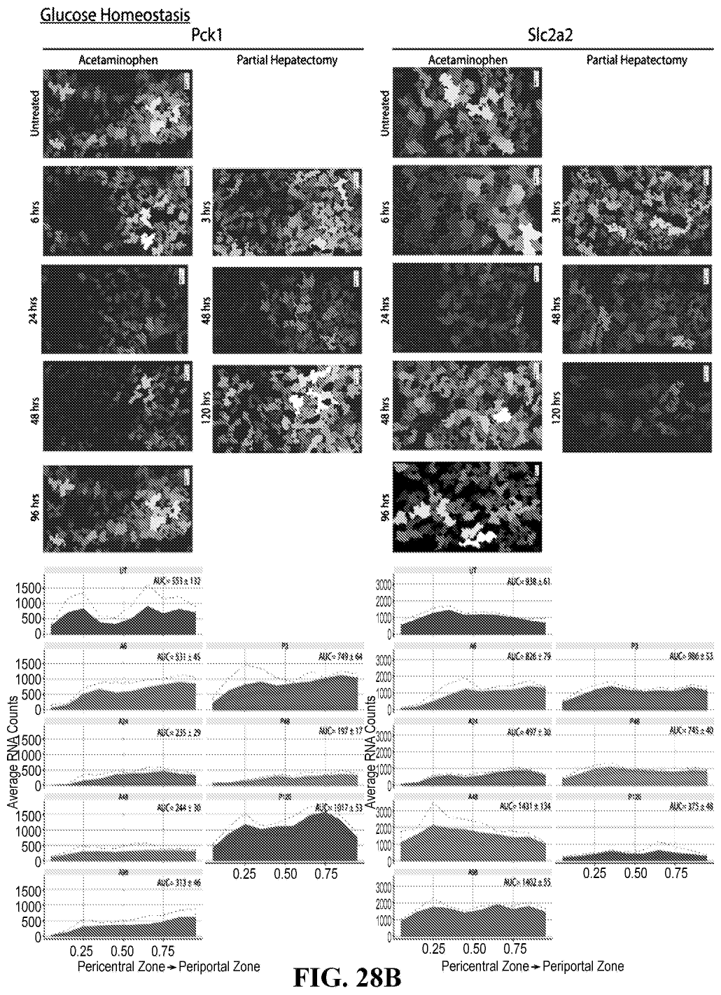

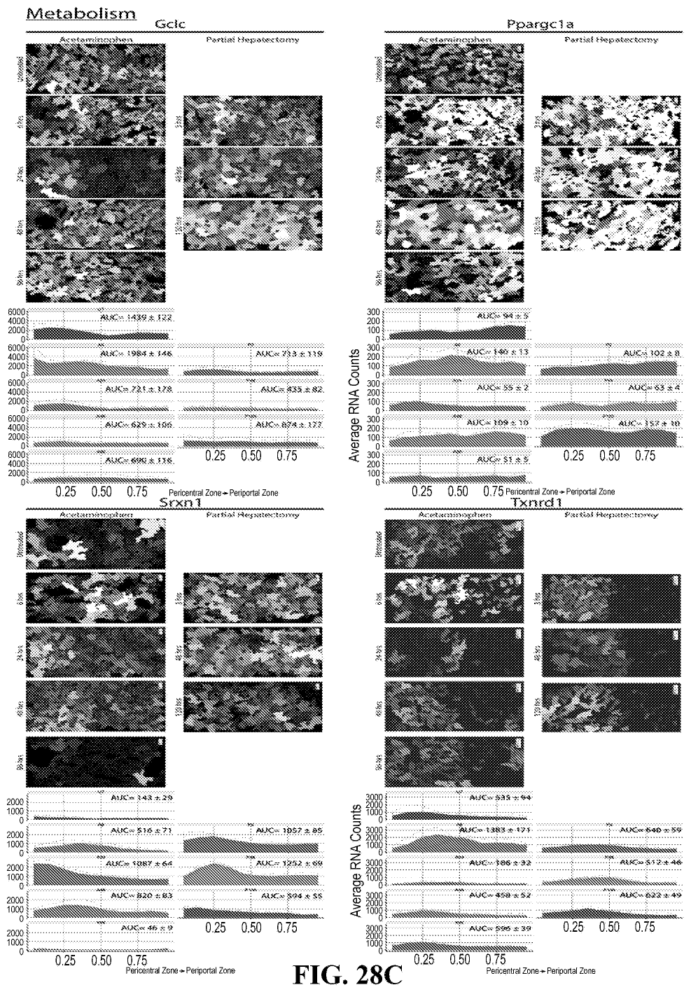

To further define shared and unique responses in both liver injury models, Applicants determined differentially expressed genes (DEG) between each treatment condition and untreated controls (UT), and then pooled results to reveal composite DEG results for APAP and PH (Methods, Table 8a, 8b), for which Applicants performed select validations using smFISH (FIG. 20, FIG. 28A-28D). A number of gene expression alterations could be attributed to injury-dependent effects. A large number of gene expression changes, however, were shared between the two models, indicating they do not reflect the specific nature of injury (FIG. 20A, 20B, Table 15a-b). Gene set analysis (GSA) revealed an enrichment of pathways involved in toxic injury within the APAP model, including GO Acute Inflammatory Response, Hallmark Xenobiotic Metabolism, Go Acute Phase Response, Go Regulation of Fatty Acid Metabolic Process, Go Organic Hydroxy Compound Metabolic Process, KEGG PPAR Signaling Pathway, Go Regulation of Extrinsic Apoptotic Signaling Pathway, Go Organic Acid Metabolic Process, Hallmark Bile Acid metabolism, Go Regulation of Steroid Metabolic Process. (FIG. 20C, Tables 16A-16C of U.S. Provisional Application 62/925,693, filed Oct. 24, 2019, and incorporated herein by reference)²⁶. The PH model, meanwhile, exhibited an enrichment among pathways involved in cell proliferation, which could be attributed to differences in the extent of injury between the two models (FIG. 20C, Tables 16A-16C of U.S. Provisional Application 62/925,693, filed Oct. 24, 2019, and incorporated herein by reference). Pathway enrichments unique to PH may include Vesicle Mediated Transport, Phospholipid Binding, Enzyme Linked Receptor Protein Signaling Pathway, Response to Growth Factor, Response to Abiotic Stimulus, Wong Adult Tissue Stem Module, Cellular Response to Stress, Regulation of Growth, Regulation of Cell Proliferation. Following APAP overdose, there was a substantial anti-oxidant response by GSA and expression of individual anti-oxidant response genes, such as thioredoxin (Txnrd1) and glutamate-cysteine ligase subunit c (Gclc) (FIG. 20D). Pathway Enrichments shared between APAP and PH may include Glycerolipid Metabolic Process, Extracellular Matrix, Platelet Degranulation, Exocytosis, Wound Healing, Negative Regulation of Peptidase Activity, Response to Biotic Stimulus, Regulation of Hydrolase Activity, Ion Homeostatis, and Enzyme Regulator Activity, associated with liver functions healing. (FIG. 13, 20C) Surprisingly, smFISH analysis revealed an up-regulation of Txnrd1 and Gclc across the entire liver lobule reaching into the periportal region (FIG. 20D). These findings suggest that some injury responses are not exclusive to the region of injury.

TABLE 15a
Shared composite DEG. Table of genes with concordant (up or down) regulation relative to untreated in
both APAP and PH. NA indicates that a particular gene was not significantly differentially expressed.

| | avg_logFC_APAP | avg_logFC_PH | p_val_adj_APAP | p_val_adj_PH | A6logFC | A24logFC |
|---|---|---|---|---|---|---|
| Mup11 | −3.2139532 | −1.76921 | 0 | 0 | −3.2139532 | −1.6114348 |
| Mup19 | −2.3963453 | −3.63217 | 0 | 0 | −2.39634529 | −1.6993527 |
| Mup17 | −2.3543586 | −2.51447 | 0 | 0 | −2.35435856 | −1.8003486 |
| Fabp1 | −1.0161748 | −0.82649 | 1.10E−198 | 0 | −1.01617485 | NA |
| Akr1c6 | −1.58626 | −0.50114 | 2.54E−187 | 6.85E−150 | −1.58626 | −0.3176388 |
| Mgst1 | −1.0601549 | −0.36875 | 2.13E−181 | 2.50E−131 | −1.06015485 | NA |
| Mup9 | −2.004164 | −2.52066 | 0 | 0 | −2.004164 | −1.8474469 |
| Apoc1 | −0.8712827 | −0.31074 | 7.90E−242 | 1.04E−90 | −0.87128274 | −0.3803368 |
| Mup16 | −1.6020855 | −1.24514 | 0 | 0 | −1.60208554 | −1.0189759 |
| Adh1 | −1.2550282 | −0.29598 | 1.90E−167 | 3.27E−106 | −1.25502821 | −0.342019 |
| Cyp2e1 | −2.3185702 | −0.3015 | 4.79E−166 | 4.23E−36 | −2.31857016 | −0.4990336 |
| Gpx1 | −0.7685563 | −0.32198 | 6.77E−238 | 2.36E−120 | −0.76855633 | −0.5079714 |
| Mup3 | −0.8062194 | −1.42376 | 0 | 0 | −0.70348317 | −0.8062194 |
| Mup18 | −1.7271783 | −2.61774 | 0 | 0 | −1.62795678 | −1.7271783 |
| Apoc4 | −1.0022658 | −0.50176 | 9.54E−150 | 1.19E−182 | −1.00226577 | −0.4284969 |
| Mup6 | −2.0320849 | −2.64537 | 0 | 0 | −1.87185204 | −1.6781848 |
| Nudt7 | −1.5642669 | −0.56604 | 2.67E−255 | 9.48E−52 | −1.56426694 | NA |
| Mup12 | −1.2699737 | −1.22808 | 1.14E−185 | 0 | −1.26997373 | NA |
| Car3 | −1.0360931 | −1.29219 | 1.61E−124 | 0 | −1.03609313 | NA |
| Apoa2 | −0.5455185 | −0.61598 | 0 | 0 | −0.54278496 | −0.5455185 |
| Selenbp2 | −1.190222 | −0.8572 | 0 | 9.70E−194 | −1.00916572 | −0.3833246 |
| 2810007J24Rik | −0.9143011 | −1.094 | 6.76E−113 | 2.63E−273 | −0.91430112 | NA |
| Uqcrq | −0.8866551 | −0.48535 | 1.50E−140 | 2.42E−124 | −0.88665513 | −0.595921 |
| Ttr | −0.5306256 | −0.28614 | 5.79E−264 | 3.87E−82 | −0.53062564 | NA |
| Cyp2c29 | −1.14963 | −1.02727 | 1.97E−99 | 2.76E−235 | −1.14962998 | −0.5860034 |
| Tmem205 | −0.7747427 | −0.36376 | 1.05E−96 | 6.01E−57 | −0.77474273 | −0.4721077 |
| Gsta3 | −0.8062905 | −0.79693 | 2.44E−166 | 3.08E−250 | −0.80629047 | −0.6693549 |
| Cyb5 | −0.7101896 | −0.75169 | 8.36E−212 | 0 | −0.71018958 | −0.7048085 |
| Hsd3b7 | −1.0059958 | −0.58164 | 3.79E−93 | 5.15E−93 | −1.0059958 | −0.3952566 |
| Acaa1b | −0.8508413 | −0.85553 | 4.51E−247 | 0 | −0.73282346 | −0.8508413 |
| Gm13775 | −1.0780882 | −1.25566 | 1.37E−292 | 0 | −1.06561886 | −1.0324519 |
| Psen2 | −0.9473738 | −0.41956 | 1.77E−85 | 1.69E−77 | −0.94737377 | NA |
| Mup15 | −1.3164641 | −1.36418 | 1.22E−233 | 5.96E−268 | −1.31646415 | −1.2349284 |
| Cyp8b1 | −0.8892298 | −0.59929 | 8.76E−88 | 1.54E−120 | −0.88922983 | −0.5536162 |
| Rps27l | −0.8659275 | −0.64582 | 5.43E−90 | 1.42E−139 | −0.86592748 | −0.5485062 |
| Cox6c | −0.8442805 | −0.61022 | 3.24E−81 | 2.30E−132 | −0.84428054 | −0.5028028 |
| Ugt2b5 | −0.6033512 | −0.71634 | 9.60E−175 | 5.77E−289 | −0.60335125 | −0.5764988 |
| Hsd3b5 | −1.0695202 | −0.88646 | 1.41E−154 | 3.58E−129 | −1.06952022 | NA |
| Cyp2a5 | −1.4989102 | −0.93818 | 3.74E−79 | 8.06E−111 | −1.49891017 | −0.681078 |
| Ugt2b1 | −0.7768169 | −0.7221 | 2.28E−76 | 5.69E−140 | −0.77681693 | −0.2894617 |
| Cyp1a2 | −0.9939358 | −0.48192 | 1.08E−75 | 3.86E−66 | −0.99393584 | NA |
| Hgd | −0.7585723 | −0.274 | 1.96E−75 | 2.46E−30 | −0.75857227 | NA |
| Adck3 | −0.8761154 | −0.56969 | 2.27E−75 | 4.38E−91 | −0.87611538 | −0.2825153 |
| Mup21 | −1.0081719 | −1.63781 | 0 | 0 | −0.82724993 | −0.6977358 |
| Cyp2d9 | −0.6406986 | −0.61668 | 3.96E−157 | 8.72E−151 | −0.64069861 | −0.5671662 |
| Apoe | −0.4212717 | −0.61588 | 0 | 0 | −0.36896653 | −0.4212717 |
| mt-Nd4 | −1.9574248 | −1.95321 | 0 | 0 | −0.63884813 | −1.9574248 |
| C8g | −0.6545103 | −0.45564 | 4.13E−96 | 2.41E−80 | −0.65451026 | −0.4819645 |
| Mup7 | −1.1155878 | −0.84773 | 0 | 8.04E−142 | −0.94137049 | NA |
| Ubc | −0.6618397 | −0.27324 | 4.04E−67 | 3.58E−41 | −0.66183972 | −0.3704876 |
| Pon1 | −0.6857513 | −0.42826 | 1.66E−66 | 1.97E−64 | −0.68575128 | −0.3227485 |
| Oaf | −0.7328674 | −0.33377 | 1.03E−122 | 3.11E−47 | −0.7328674 | NA |
| Ugt2b36 | −0.5834315 | −0.62967 | 1.36E−63 | 1.10E−145 | −0.58343149 | −0.3293162 |
| Ces3a | −0.5253364 | −0.72101 | 5.80E−77 | 1.41E−206 | −0.52533635 | −0.3687356 |
| Mup5 | −1.3706213 | −1.77461 | 1.94E−240 | 0 | −1.23728834 | −1.3706213 |
| Ndufa4 | −0.6831267 | −0.51272 | 3.70E−65 | 4.13E−107 | −0.6831267 | −0.4433111 |
| Hagh | −0.6740097 | −0.39811 | 1.62E−61 | 4.45E−73 | −0.67400971 | NA |
| Cyp3a11 | −0.4231307 | −0.80355 | 1.05E−122 | 0 | −0.42313075 | −0.3638912 |
| Hsd3b3 | −0.7795011 | −0.34746 | 1.98E−61 | 3.82E−42 | −0.77950112 | NA |
| Hint1 | −0.6738806 | −0.48702 | 1.71E−80 | 9.22E−101 | −0.67388058 | −0.4736653 |
| Scp2 | −0.5191249 | −0.31952 | 3.33E−100 | 5.16E−159 | −0.51912492 | NA |
| Cox4i1 | −0.5159892 | −0.57523 | 4.62E−112 | 2.23E−168 | −0.51598917 | −0.4542436 |
| Sod1 | −0.4393198 | −0.48337 | 3.30E−72 | 3.17E−216 | −0.4393198 | −0.3036291 |
| Hacl1 | −0.7068977 | −0.3329 | 1.20E−59 | 4.71E−52 | −0.70689773 | NA |
| Hpd | −0.5181804 | −0.31794 | 2.47E−59 | 2.00E−54 | −0.51818039 | −0.313324 |
| mt-Co2 | −1.3965432 | −1.41733 | 7.79E−219 | 0 | −1.10533286 | −1.3965432 |
| Cyp2c37 | −0.8869038 | −0.82039 | 9.93E−57 | 2.10E−130 | −0.88690378 | −0.4875226 |
| Inmt | −0.7310164 | −0.67979 | 1.21E−55 | 1.56E−139 | −0.7310164 | −0.4435295 |
| Uox | −0.5531155 | −0.48042 | 6.13E−55 | 1.42E−89 | −0.55311551 | NA |
| Uqcr11 | −0.60308 | −0.36807 | 1.37E−54 | 8.58E−61 | −0.60307999 | −0.2632414 |
| Ankrd55 | −1.8735659 | −1.95094 | 4.38E−235 | 2.52E−247 | −1.25083463 | −1.8735659 |
| Prdx1 | −0.5292291 | −0.35376 | 1.10E−92 | 2.27E−73 | −0.52922911 | −0.2676651 |
| Hebp1 | −0.6354323 | −0.31301 | 1.18E−51 | 1.26E−40 | −0.63543227 | NA |
| Cox7b | −0.6422408 | −0.65009 | 3.42E−51 | 3.36E−144 | −0.64224078 | −0.2673975 |
| mt-Nd1 | −1.4196836 | −1.544 | 0 | 0 | −0.38833608 | −1.4196836 |
| Cox7a2 | −0.6503384 | −0.4411 | 2.00E−48 | 3.27E−59 | −0.65033839 | −0.363458 |
| Chchd2 | −0.5525035 | −0.51862 | 7.88E−56 | 1.10E−119 | −0.55250349 | −0.3949168 |
| Rarres2 | −0.5521946 | −0.49201 | 3.29E−48 | 1.76E−89 | −0.55219458 | −0.2666165 |
| Ndufa6 | −0.61365 | −0.41844 | 4.43E−48 | 3.80E−59 | −0.61364998 | −0.3068153 |
| Rdh7 | −0.418626 | −0.49691 | 6.63E−181 | 5.58E−132 | −0.41862596 | −0.2690433 |
| Pah | −0.522077 | −0.37985 | 1.25E−47 | 8.65E−60 | −0.52207695 | NA |
| Leap2 | −0.6713009 | −0.40479 | 1.25E−47 | 1.27E−50 | −0.67130094 | −0.2747781 |
| Cyp7b1 | −0.6580227 | −0.43816 | 3.09E−98 | 1.37E−54 | −0.65802273 | NA |
| Cyp2c50 | −0.6618233 | −0.76391 | 2.92E−46 | 8.52E−186 | −0.6618233 | −0.2904835 |
| Mup4 | −1.0870385 | −1.94565 | 9.25E−125 | 0 | −0.99530818 | −1.0870385 |
| Cml1 | −0.6142405 | −0.47269 | 1.70E−43 | 5.55E−63 | −0.61424052 | −0.3269095 |
| Aadac | −0.619958 | −0.74734 | 4.18E−43 | 6.76E−122 | −0.61995803 | −0.4037666 |
| Mup13 | −0.7757431 | −0.87508 | 2.45E−76 | 6.29E−165 | −0.77574306 | −0.6948897 |
| Ttc39c | −0.6362749 | −0.28961 | 2.03E−42 | 2.34E−27 | −0.63627493 | NA |
| Aldh1a1 | −0.4787974 | −0.40245 | 1.02E−41 | 1.39E−102 | −0.47879745 | NA |
| Ddt | −0.5222211 | −0.34584 | 1.33E−60 | 1.40E−46 | −0.52222115 | −0.4224109 |
| Cox6a1 | −0.4872287 | −0.39062 | 1.78E−87 | 1.67E−61 | −0.48722865 | −0.3666972 |
| Atp5j2 | −0.5130805 | −0.40958 | 3.32E−40 | 3.23E−66 | −0.51308051 | NA |
| Ndufb2 | −0.5771545 | −0.33686 | 1.66E−45 | 1.21E−30 | −0.57715446 | −0.3412263 |
| Ndufb9 | −0.5038288 | −0.4837 | 5.48E−40 | 1.07E−104 | −0.50382881 | NA |
| Atp5h | −0.6398091 | −0.5555 | 1.41E−124 | 7.99E−68 | −0.6398091 | −0.5794989 |
| Qdpr | −0.5235738 | −0.3166 | 1.45E−39 | 1.80E−33 | −0.52357376 | NA |
| Atp5f1 | −0.5170612 | −0.46071 | 2.05E−39 | 1.10E−77 | −0.51706117 | −0.2872403 |
| Serpina1a | −0.675202 | −0.35537 | 4.32E−247 | 6.23E−81 | −0.37845707 | −0.675202 |
| Fabp2 | −0.6155693 | −0.52719 | 4.18E−38 | 2.63E−76 | −0.61556934 | −0.3921961 |
| Khk | −0.4914532 | −0.53015 | 1.03E−67 | 8.89E−91 | −0.49145322 | −0.297617 |
| Mpc2 | −0.4828394 | −0.39521 | 7.20E−37 | 2.71E−68 | −0.48283942 | NA |
| Ndufb6 | −0.5076826 | −0.51838 | 4.30E−35 | 3.96E−85 | −0.50768256 | −0.3064033 |
| Elovl3 | −0.675967 | −0.92485 | 6.30E−121 | 1.57E−229 | −0.50672414 | −0.675967 |
| Arl4d | −0.5072283 | −0.32852 | 6.71E−35 | 5.36E−30 | −0.50722832 | NA |
| Akr1c14 | −0.5185708 | −0.28017 | 1.08E−34 | 1.20E−21 | −0.5185708 | NA |
| Csad | −0.559786 | −1.21486 | 1.92E−72 | 5.27E−267 | −0.55978602 | −0.5312525 |
| Ndufb11 | −0.5096711 | −0.47904 | 8.28E−53 | 2.10E−70 | −0.50967106 | −0.4275253 |
| Cox5b | −0.4987836 | −0.39369 | 4.68E−34 | 3.39E−45 | −0.49878362 | −0.337825 |
| Rplp1 | −0.504663 | −0.27482 | 4.91E−34 | 6.89E−20 | −0.50466296 | −0.2866404 |
| Fxyd1 | −0.4799084 | −0.27342 | 3.83E−34 | 2.95E−25 | −0.4799084 | −0.3117591 |
| Atp5j | −0.4837353 | −0.41538 | 3.59E−33 | 7.16E−66 | −0.48373528 | NA |
| Rpl32 | −0.5322184 | −0.49859 | 1.10E−32 | 4.42E−73 | −0.53221838 | −0.2549101 |
| Dio1 | −0.5452837 | −0.46973 | 1.11E−32 | 9.15E−59 | −0.54528374 | NA |
| Nit2 | −0.4859406 | −0.25532 | 4.49E−32 | 6.79E−25 | −0.48594061 | NA |
| Gnmt | −0.4603611 | −0.28818 | 1.33E−133 | 8.65E−84 | −0.33340449 | −0.4603611 |
| Ssr4 | −0.5075772 | −0.28316 | 1.87E−31 | 7.71E−23 | −0.50757724 | NA |
| Ndufs6 | −0.5379298 | −0.34104 | 2.00E−31 | 2.06E−29 | −0.53792983 | −0.359161 |
| Etfb | −0.3654903 | −0.27001 | 4.01E−31 | 4.81E−54 | −0.36549028 | NA |
| Rsp14 | −0.402093 | −0.60751 | 1.07E−73 | 3.76E−152 | −0.40209304 | −0.292251 |
| Blvrb | −0.5451127 | −0.35108 | 6.23E−30 | 2.20E−30 | −0.54511272 | NA |
| Uqcr10 | −0.4519019 | −0.38232 | 2.60E−29 | 1.93E−47 | −0.45190185 | NA |
| Cyp3a25 | −0.3860949 | −0.41646 | 1.19E−134 | 5.44E−129 | −0.38609486 | NA |
| Mup8 | −0.795757 | −0.82996 | 1.23E−114 | 3.80E−123 | −0.75257771 | −0.6133344 |
| Sc5d | −0.5009009 | −0.27429 | 3.57E−48 | 3.96E−19 | −0.50090092 | 0.34501688 |
| Adipor2 | −0.4737915 | −0.25255 | 3.14E−27 | 1.11E−22 | −0.47379151 | NA |
| Rpl36a1 | −0.4755414 | −0.33184 | 1.26E−26 | 3.44E−26 | −0.47554139 | NA |
| Serpina1e | −0.6620459 | −0.7252 | 0 | 0 | −0.26841379 | −0.2843446 |
| Serpina1d | −0.4940072 | −0.58685 | 1.74E−184 | 5.86E−199 | −0.28901122 | −0.4940072 |
| Dcxr | −0.4408516 | −0.27134 | 2.61E−43 | 1.59E−18 | −0.44085162 | −0.4106665 |
| Cmbl | −0.4427064 | −0.26537 | 1.48E−28 | 9.66E−25 | −0.44270644 | −0.312422 |
| Ndufb4 | −0.453693 | −0.33169 | 3.64E−28 | 2.14E−33 | −0.45369304 | −0.3251455 |
| B2m | −0.4179739 | −0.43085 | 1.86E−102 | 2.11E−97 | −0.33035492 | −0.4179739 |
| Cyp4a12a | −0.4568275 | −0.47604 | 6.69E−26 | 1.00E−65 | −0.45682749 | NA |
| Hspe1 | −0.4807349 | −0.25791 | 9.37E−26 | 3.13E−12 | −0.48073495 | −0.290583 |
| Uqcrh | −0.4291645 | −0.48311 | 1.41E−25 | 5.89E−83 | −0.42916452 | NA |
| mt-Co3 | −1.1670277 | −1.23396 | 4.34E−176 | 1.47E−287 | −0.70593061 | −1.1670277 |
| Minos1 | −0.455548 | −0.3084 | 2.00E−25 | 8.00E−23 | −0.45554803 | NA |
| Mup14 | −0.7607669 | 1 | 2.64E−96 | 8.52E−68 | −0.69746981 | NA |
| Cfhr2 | −0.4484632 | −0.63062 | 2.09E−34 | 7.59E−98 | −0.44846316 | −0.3219287 |
| Timm13 | −0.4329618 | −0.26627 | 2.46E−24 | 1.44E−18 | −0.4329618 | NA |
| 1500017E21Rik | −0.4609972 | −0.51793 | 2.47E−24 | 1.93E−69 | −0.46099722 | NA |
| Hint2 | −0.4243211 | −0.37377 | 6.90E−37 | 2.60E−44 | −0.4243211 | −0.3701685 |
| Pbld1 | −0.442543 | −0.26463 | 4.53E−92 | 2.23E−23 | −0.44254299 | NA |
| Glo1 | −0.406574 | −0.37923 | 1.87E−23 | 3.51E−47 | −0.40657402 | NA |
| Rpl13a | −0.3814621 | −0.48017 | 3.58E−22 | 2.24E−99 | −0.38146214 | NA |
| Ces3b | −0.3663797 | −0.81956 | 5.30E−27 | 6.86E−161 | −0.36637965 | −0.2866854 |
| Rps18 | −0.4885408 | −0.41508 | 8.18E−22 | 1.19E−34 | −0.4885408 | NA |
| Serpina12 | −0.4533994 | −0.61237 | 2.90E−24 | 7.52E−89 | −0.45339936 | NA |
| Ndufa2 | −0.3964736 | −0.30831 | 2.46E−21 | 9.67E−23 | −0.39647363 | NA |
| Mup1 | 1 | −0.88291 | 7.40E−25 | 1.78E−91 | −0.73772733 | −0.2618377 |
| Bphl | −0.4074792 | −0.29948 | 5.02E−21 | 1.70E−33 | −0.40747923 | NA |
| Prdx4 | −0.4175889 | −0.37802 | 6.96E−24 | 3.39E−40 | −0.41758889 | −0.3074421 |
| Cyp2c54 | −0.3949376 | −0.71286 | 9.37E−37 | 1.75E−163 | −0.39493764 | −0.3268597 |
| Rps2 | −0.3878627 | −0.47452 | 5.09E−44 | 4.24E−69 | −0.38786266 | −0.3844542 |
| Acsl1 | −0.370317 | −0.56803 | 1.12E−70 | 3.63E−104 | −0.34342401 | −0.370317 |
| Cebpa | −0.391553 | −0.48161 | 3.89E−20 | 2.92E−66 | −0.39155301 | NA |
| Uqcrb | −0.4084167 | −0.35966 | 6.59E−20 | 6.07E−33 | −0.40841674 | NA |
| 1110008F13Rik | −0.4552706 | −0.32748 | 4.13E−58 | 1.42E−34 | −0.37509166 | −0.4552706 |
| Tmem243 | −0.3790533 | −0.27511 | 2.35E−20 | 2.06E−22 | −0.37905328 | −0.2655515 |
| Atp5g3 | −0.3002218 | −0.35725 | 4.43E−19 | 1.87E−78 | −0.30022182 | NA |
| Rps15 | −0.455155 | −0.32454 | 7.79E−24 | 3.43E−16 | −0.45515504 | −0.3561377 |
| Nme1 | −0.4328853 | −0.29161 | 5.43E−19 | 9.48E−21 | −0.43288531 | NA |
| Timm8b | −0.3935819 | −0.25099 | 8.39E−19 | 2.42E−15 | −0.3935819 | NA |
| Ndufc2 | −0.4252848 | −0.37134 | 1.02E−18 | 1.56E−33 | −0.42528482 | −0.3080394 |
| S100a1 | −0.3761862 | −0.35554 | 1.76E−35 | 2.21E−35 | −0.37433812 | −0.3761862 |
| Sdhd | −0.3352613 | −0.3023 | 8.20E−61 | 2.47E−32 | −0.33526125 | NA |
| Usmg5 | −0.4304948 | −0.28365 | 4.55E−56 | 1.10E−10 | −0.43049483 | NA |
| Cyp27a1 | −0.3735103 | −0.32623 | 4.44E−19 | 1.46E−36 | −0.37351033 | −0.2515784 |
| Rps3 | −0.3493208 | −0.3399 | 5.25E−18 | 7.09E−57 | −0.34932075 | NA |
| Slc22a30 | −0.3745984 | −0.33187 | 5.26E−18 | 7.77E−38 | −0.3745984 | NA |
| Rps9 | −0.3717938 | −0.30861 | 1.94E−22 | 6.38E−21 | −0.3717938 | −0.3139512 |
| Oaz1 | −0.4185288 | −0.42629 | 1.75E−59 | 1.27E−51 | −0.34722806 | −0.374798 |
| Ndufb8 | −0.3480766 | −0.3899 | 3.81E−17 | 1.46E−55 | −0.34807658 | NA |
| Hist1h1c | −0.3009065 | −0.33105 | 1.17E−16 | 5.78E−33 | −0.30090653 | NA |
| Ces1f | −0.31259 | −0.53364 | 1.45E−16 | 9.25E−112 | −0.31258996 | NA |
| Slco1a1 | −0.3628438 | −0.63248 | 1.36E−29 | 5.52E−101 | −0.36284376 | NA |
| Ugt2a3 | −0.3334414 | −0.5474 | 8.21E−34 | 2.33E−87 | −0.30752809 | −0.3334414 |
| Psmb1 | −0.3584824 | −0.27157 | 3.95E−16 | 5.68E−21 | −0.35848235 | NA |
| Baat | −0.308582 | −0.44583 | 4.31E−16 | 1.01E−65 | −0.308582 | NA |
| Bckdha | −0.3499796 | −0.4047 | 6.82E−16 | 2.27E−47 | −0.34997961 | NA |
| Atp5o | −0.3316283 | −0.26865 | 9.93E−16 | 2.25E−21 | −0.33162829 | NA |
| C8b | −0.3601712 | −0.25108 | 1.12E−72 | 1.50E−25 | −0.31591777 | 0.31870312 |
| Mrps18c | −0.3662911 | −0.27427 | 1.60E−15 | 1.08E−17 | −0.3662911 | NA |
| Sdhb | −0.2896428 | −0.35736 | 2.09E−15 | 2.51E−46 | −0.28964282 | NA |
| Aox3 | −0.3333198 | −0.42946 | 2.60E−25 | 1.72E−66 | −0.33331983 | 0.28784543 |
| Mdh1 | −0.3076578 | −0.32903 | 3.99E−15 | 2.04E−40 | −0.30765782 | NA |
| Otc | −0.2990342 | −0.58497 | 6.99E−15 | 1.50E−116 | −0.29903416 | NA |
| Sc4mol | −0.3261705 | −0.51957 | 6.26E−23 | 1.06E−80 | −0.32617048 | −0.2734797 |
| Igfbp2 | −0.3450878 | −0.71783 | 4.02E−44 | 3.60E−190 | −0.29898103 | −0.3450878 |
| Rps21 | −0.3644509 | −0.31687 | 2.02E−14 | 2.81E−22 | −0.36445087 | NA |
| Sar1b | −0.3176075 | −0.42209 | 1.79E−35 | 6.58E−68 | −0.30793071 | −0.3176075 |
| Slc2a2 | −0.3123484 | −0.29127 | 5.54E−60 | 2.49E−31 | −0.31234839 | NA |
| Txn1 | −0.3091238 | −0.27735 | 2.50E−14 | 6.45E−28 | −0.30912379 | NA |
| 0610011F06Rik | −0.3036885 | −0.28594 | 2.80E−14 | 3.51E−28 | −0.30368851 | NA |
| Slc10a1 | −0.3257574 | −0.39549 | 1.24E−84 | 8.15E−51 | −0.32575741 | NA |
| Rps10 | −0.3369091 | −0.25266 | 4.59E−14 | 3.85E−13 | −0.33690905 | NA |
| Esd | −0.28076 | −0.38154 | 9.90E−24 | 1.94E−49 | −0.28076002 | −0.2537949 |
| Cyp4f14 | −0.2959332 | −0.28357 | 1.60E−13 | 6.47E−28 | −0.29593321 | NA |
| Dhrs4 | −0.3320744 | −0.26886 | 9.59E−13 | 5.96E−15 | −0.33207442 | NA |
| Mup2 | 1 | −0.73872 | 8.16E−38 | 5.33E−115 | −0.50447051 | −0.2742436 |
| Selk | −0.3383144 | −0.27925 | 1.30E−13 | 4.58E−18 | −0.3383144 | NA |
| Rpl14 | −0.2776378 | −0.35706 | 7.87E−12 | 2.16E−37 | −0.27763775 | NA |
| Ndufs4 | −0.3131609 | −0.29996 | 1.01E−11 | 2.26E−18 | −0.31316093 | NA |
| Abcd3 | −0.2718051 | −0.32684 | 1.44E−11 | 1.45E−40 | −0.27180511 | NA |
| Cela1 | −0.2722968 | −0.25261 | 1.78E−11 | 1.03E−30 | −0.27229684 | NA |
| Sparcl1 | −0.253617 | −0.25362 | 3.40E−53 | 3.78E−57 | −0.25361699 | −0.253617 |
| Aadat | −0.318628 | −0.32573 | 3.04E−11 | 9.13E−34 | −0.31862795 | NA |
| Agxt | −0.2583834 | −0.30383 | 4.28E−11 | 1.79E−39 | −0.25838339 | NA |
| Sigmar1 | −0.3054683 | −0.28236 | 7.64E−11 | 5.67E−17 | −0.30546833 | NA |
| Psma6 | −0.3035366 | −0.28171 | 8.48E−11 | 2.82E−20 | −0.30353658 | NA |
| Cyp2c55 | −0.2904354 | −0.34784 | 9.36E−11 | 2.21E−52 | −0.29043537 | NA |
| Proc | −0.2707194 | −0.31978 | 9.66E−11 | 1.66E−39 | −0.27071936 | NA |
| Cebpb | −0.4715795 | −0.30633 | 2.38E−50 | 1.73E−23 | −0.36173829 | −0.4715795 |
| Rpl13 | −0.2996936 | −0.31461 | 1.78E−21 | 7.45E−64 | −0.29881609 | −0.2996936 |
| Ubb | −0.2750304 | −0.26377 | 2.58E−11 | 0.0138701 | −0.27503038 | −0.2593394 |
| Rps24 | −0.2624737 | −0.38646 | 8.21E−10 | 6.91E−41 | −0.26247367 | NA |
| Ppp2r5a | −0.2605045 | −0.3151 | 7.14E−52 | 3.37E−36 | −0.26050454 | NA |
| Lipc | −0.2548415 | −0.42306 | 1.09E−09 | 3.19E−59 | −0.25484148 | NA |
| Rabac1 | −0.2659872 | −0.34206 | 1.45E−09 | 3.25E−38 | −0.26598721 | NA |
| Mvd | −0.2704099 | −0.32733 | 1.91E−09 | 2.22E−59 | −0.27040987 | NA |
| Zfp683 | −0.4216891 | −0.42169 | 1.05E−25 | 1.10E−47 | −0.42168911 | −0.421395 |
| Park7 | −0.26023 | −0.27093 | 1.55E−56 | 9.02E−25 | −0.26023002 | NA |
| Cyp2b10 | −1.1644909 | −1.25497 | 9.40E−117 | 1.74E−217 | −0.51746066 | −1.1644909 |
| Rpl34 | −0.2975985 | −0.26459 | 8.13E−08 | 1.43E−12 | −0.29759854 | NA |
| mt-Atp6 | −0.573743 | −0.91464 | 1.31E−25 | 7.98E−137 | −0.47818742 | −0.573743 |
| Sco2 | −0.2928706 | −0.39595 | 1.32E−23 | 1.04E−49 | −0.26050852 | −0.2928706 |
| Ndufa5 | −0.251989 | −0.31028 | 1.96E−06 | 4.19E−22 | −0.25198899 | NA |
| Serpina3c | −0.472511 | −0.59557 | 8.19E−59 | 4.68E−124 | −0.28701133 | −0.4088789 |
| 4931406C07Rik | −0.2543636 | −0.28178 | 9.62E−06 | 3.10E−19 | −0.25436363 | NA |
| Gstp1 | −0.8558314 | −0.67166 | 1.88E−150 | 4.28E−90 | −0.26437467 | −0.4301974 |
| Ftl1 | 1 | −0.34781 | 2.43E−17 | 4.66E−11 | −0.26029441 | −0.3497774 |
| mt-Nd2 | −1.241926 | −1.35078 | 0 | 0 | NA | −1.241926 |
| mt-Nd5 | −1.3835911 | −1.49646 | 0 | 0 | NA | −1.3835911 |
| mt-Co1 | −1.4334276 | −1.56995 | 0 | 0 | NA | −1.4334276 |
| mt-Cytb | −1.5712329 | −1.55403 | 0 | 0 | NA | −1.5712329 |
| mt-Rnr2 | −0.778342 | −0.96745 | 5.04E−251 | 0 | NA | −0.778342 |
| Serpina1e | −0.4026267 | −0.27984 | 7.17E−151 | 1.24E−63 | NA | −0.4026267 |
| mt-Rnr1 | −0.4853999 | −0.87219 | 1.71E−195 | 0 | NA | −0.4562114 |
| Ahcy | −0.5629686 | −0.49011 | 6.43E−120 | 1.57E−115 | NA | −0.5629686 |
| Serpina1b | −0.3017706 | −0.29448 | 4.66E−97 | 1.99E−74 | NA | −0.3017706 |
| Ces1d | −0.428906 | −1.2015 | 8.07E−91 | 0 | NA | −0.428906 |
| Cth | −0.5108991 | −0.33921 | 1.34E−76 | 3.95E−48 | NA | −0.5108991 |
| Ech1 | −0.4291326 | −0.50402 | 5.62E−72 | 2.34E−114 | NA | −0.4291326 |
| Ephx2 | −0.3693036 | −0.5708 | 3.91E−109 | 1.49E−121 | NA | −0.3693036 |
| mt-Nd6 | −0.4778511 | −0.68551 | 1.67E−57 | 2.74E−110 | NA | −0.4778511 |
| Cyp2d10 | −0.3231514 | −0.32533 | 1.31E−55 | 3.76E−49 | NA | −0.3231514 |
| Clpx | −0.4047356 | −0.41084 | 1.07E−46 | 1.11E−53 | NA | −0.4047356 |
| Gm4076 | −0.3254694 | −0.34583 | 2.57E−39 | 7.98E−74 | NA | −0.3254694 |
| Acot1 | 0.30784004 | −0.29933 | 1.49E−100 | 2.65E−38 | −0.28863865 | −0.2862042 |
| Gm5096 | −0.3630891 | −0.39955 | 1.95E−32 | 4.99E−39 | NA | −0.3630891 |
| Gas5 | −0.3361532 | −0.29842 | 2.74E−31 | 6.31E−32 | NA | −0.3361532 |
| Mup20 | −0.4762073 | −1.23782 | 3.88E−128 | 0 | NA | −0.2818717 |
| Akr1c19 | −0.2726671 | −0.38253 | 7.78E−21 | 1.32E−46 | NA | −0.2726671 |
| Eci1 | −0.2763831 | −0.31335 | 2.48E−20 | 3.71E−30 | NA | −0.2763831 |
| Fcgrt | −0.2693142 | −0.27095 | 4.12E−64 | 1.81E−26 | NA | −0.2693142 |
| Gm20594 | −0.3600006 | −0.57378 | 1.55E−09 | 4.48E−60 | 0.295686612 | −0.3600006 |
| Eef1b2 | −0.3173232 | −0.36733 | 2.36E−38 | 1.90E−45 | NA | NA |
| Rps8 | −0.2746013 | −0.27985 | 8.37E−12 | 2.52E−16 | NA | NA |
| C6 | −0.2669877 | −0.2551 | 3.03E−10 | 2.04E−21 | NA | NA |

| | A48A96logFC | A6p | A24p | A48A96p | PH3logFC |
|---|---|---|---|---|---|
| Mup11 | −1.6211346 | 1.04E−235 | 0 | 0 | −0.976756 |
| Mup19 | −1.7308682 | 4.33E−227 | 0 | 0 | −2.932276 |
| Mup17 | −2.0963504 | 1.37E−209 | 0 | 0 | −2.514473 |
| Fabp1 | NA | 1.10E−198 | NA | NA | NA |
| Akr1c6 | −0.4689885 | 2.54E−187 | 2.86E−52 | 8.38E−143 | −0.434922 |
| Mgst1 | NA | 2.13E−181 | NA | NA | −0.322743 |
| Mup9 | −1.9358842 | 9.10E−177 | 0 | 0 | −2.453597 |
| Apoc1 | 0.39209541 | 1.40E−171 | 2.01E−117 | 7.90E−242 | NA |
| Mup16 | −1.1006815 | 1.22E−168 | 2.06E−200 | 0 | −0.848727 |
| Adh1 | NA | 1.90E−167 | 8.71E−80 | NA | −0.295981 |
| Cyp2e1 | NA | 4.79E−166 | 3.18E−51 | NA | −0.301498 |
| Gpx1 | NA | 9.80E−161 | 6.77E−238 | NA | NA |
| Mup3 | −0.379596 | 1.31E−157 | 0 | 7.73E−237 | −0.52641 |
| Mup18 | −1.59087 | 4.19E−157 | 0 | 0 | −2.617739 |
| Apoc4 | NA | 9.54E−150 | 3.56E−114 | NA | −0.356086 |
| Mup6 | −2.0320849 | 2.16E−149 | 1.18E−255 | 0 | −2.386597 |
| Nudt7 | −1.1415754 | 1.44E−129 | NA | 2.67E−255 | NA |
| Mup12 | −0.767699 | 4.63E−127 | NA | 1.14E−185 | −0.589619 |
| Car3 | −0.4053086 | 1.61E−124 | NA | 4.54E−111 | NA |
| Apoa2 | NA | 6.54E−122 | 0 | NA | NA |
| Selenbp2 | −1.190222 | 6.93E−118 | 5.26E−63 | 0 | NA |
| 2810007J24Rik | NA | 6.76E−113 | NA | NA | NA |
| Uqcrq | NA | 4.23E−106 | 1.50E−140 | NA | −0.316452 |
| Ttr | 0.38890744 | 3.44E−101 | NA | 5.79E−264 | NA |
| Cyp2c29 | NA | 1.97E−99 | 2.74E−69 | NA | −1.027268 |
| Tmem205 | NA | 1.21E−96 | 1.05E−96 | NA | NA |
| Gsta3 | NA | 6.16E−95 | 2.44E−166 | NA | −0.537842 |
| Cyb5 | NA | 7.09E−94 | 8.36E−212 | NA | −0.719657 |
| Hsd3b7 | −0.3827383 | 3.79E−93 | 2.93E−47 | 1.78E−61 | −0.451728 |
| Acaa1b | NA | 9.23E−93 | 4.51E−247 | NA | −0.263859 |
| Gm13775 | −1.0780882 | 3.12E−88 | 3.97E−187 | 1.37E−292 | −1.255657 |
| Psen2 | NA | 1.77E−85 | NA | NA | −0.419556 |
| Mup15 | −1.2612126 | 1.10E−84 | 1.08E−153 | 1.22E−233 | −1.07441 |
| Cyp8b1 | 0.28231507 | 6.32E−84 | 8.76E−88 | 3.14E−45 | −0.364571 |
| Rps27l | NA | 1.39E−81 | 5.43E−90 | NA | −0.361139 |
| Cox6c | NA | 3.24E−81 | 2.39E−77 | NA | −0.411283 |
| Ugt2b5 | NA | 3.90E−81 | 9.60E−175 | NA | −0.423085 |
| Hsd3b5 | −0.8301459 | 2.04E−80 | NA | 1.41E−154 | −0.587529 |
| Cyp2a5 | NA | 3.74E−79 | 3.04E−57 | NA | −0.938183 |
| Ugt2b1 | NA | 2.28E−76 | 4.20E−34 | NA | NA |
| Cyp1a2 | −0.4457662 | 1.08E−75 | NA | 2.09E−66 | −0.48192 |
| Hgd | NA | 1.96E−75 | NA | NA | NA |
| Adck3 | NA | 2.27E−75 | 6.63E−24 | NA | −0.48294 |
| Mup21 | −1.0081719 | 2.40E−75 | 2.15E−121 | 0 | −0.668771 |
| Cyp2d9 | NA | 2.15E−73 | 3.96E−157 | NA | NA |
| Apoe | 0.4072569 | 3.19E−71 | 3.30E−238 | 0 | −0.262098 |
| mt-Nd4 | −0.9364059 | 7.92E−71 | 0 | 0 | −1.630375 |
| C8g | NA | 3.81E−68 | 4.13E−96 | NA | NA |
| Mup7 | −1.1155878 | 1.78E−67 | NA | 0 | −0.293978 |
| Ubc | NA | 4.04E−67 | 2.42E−49 | NA | −0.273243 |
| Pon1 | NA | 1.66E−66 | 5.87E−44 | NA | −0.302819 |
| Oaf | 0.40356761 | 4.77E−65 | NA | 1.03E−122 | −0.333767 |
| Ugt2b36 | NA | 1.36E−63 | 1.07E−57 | NA | −0.287193 |
| Ces3a | NA | 1.00E−62 | 5.80E−77 | NA | NA |
| Mup5 | −1.3518107 | 1.04E−61 | 6.49E−171 | 1.94E−240 | −1.484561 |
| Ndufa4 | NA | 1.11E−61 | 3.70E−65 | NA | NA |
| Hagh | NA | 1.62E−61 | NA | NA | NA |
| Cyp3a11 | 0.31392733 | 1.90E−61 | 1.05E−122 | 2.07E−107 | −0.25122 |
| Hsd3b3 | −0.2996621 | 1.98E−61 | NA | 3.00E−20 | NA |
| Hint1 | NA | 4.75E−61 | 1.71E−80 | NA | NA |
| Scp2 | −0.3251618 | 1.05E−60 | NA | 3.33E−100 | −0.279911 |
| Cox4i1 | NA | 5.83E−60 | 4.62E−112 | NA | −0.288035 |
| Sod1 | NA | 7.55E−60 | 3.30E−72 | NA | NA |
| Hacl1 | NA | 1.20E−59 | NA | NA | −0.3329 |
| Hpd | NA | 2.47E−59 | 1.72E−56 | NA | NA |
| mt-Co2 | −0.9187845 | 3.38E−58 | 7.79E−219 | 1.79E−173 | −1.341687 |
| Cyp2c37 | NA | 9.93E−57 | 9.75E−56 | NA | −0.662336 |
| Inmt | −0.2532218 | 1.21E−55 | 1.57E−51 | 7.77E−21 | −0.308364 |
| Uox | NA | 6.13E−55 | NA | NA | NA |
| Uqcr11 | NA | 1.37E−54 | 5.87E−27 | NA | NA |
| Ankrd55 | −0.9742804 | 1.82E−54 | 4.38E−235 | 1.45E−68 | −1.464696 |
| Prdx1 | −0.3618909 | 7.29E−52 | 1.94E−38 | 1.10E−92 | NA |
| Hebp1 | NA | 1.18E−51 | NA | NA | −0.313009 |
| Cox7b | NA | 3.42E−51 | 4.55E−23 | NA | −0.304639 |
| mt-Nd1 | −0.5916468 | 2.80E−49 | 0 | 5.29E−267 | −1.136737 |
| Cox7a2 | NA | 2.00E−48 | 5.69E−35 | NA | NA |
| Chchd2 | NA | 2.39E−48 | 7.88E−56 | NA | NA |
| Rarres2 | NA | 3.29E−48 | 1.17E−31 | NA | NA |
| Ndufa6 | NA | 4.43E−48 | 4.71E−27 | NA | −0.252724 |
| Rdh7 | 0.36416156 | 7.41E−48 | 1.69E−54 | 6.63E−181 | NA |
| Pah | NA | 1.25E−47 | NA | NA | NA |
| Leap2 | NA | 1.25E−47 | 1.45E−22 | NA | NA |
| Cyp7b1 | −0.5424035 | 4.96E−47 | NA | 3.09E−98 | NA |
| Cyp2c50 | NA | 2.92E−46 | 1.48E−24 | NA | −0.763912 |
| Mup4 | −0.9531018 | 1.26E−43 | 1.96E−121 | 9.25E−125 | −1.805302 |
| Cml1 | NA | 1.70E−43 | 2.10E−29 | NA | −0.27734 |
| Aadac | NA | 4.18E−43 | 5.65E−43 | NA | NA |
| Mup13 | −0.5525594 | 8.39E−43 | 2.45E−76 | 6.20E−45 | −0.755608 |
| Ttc39c | −0.3745045 | 2.03E−42 | NA | 2.88E−40 | NA |
| Aldh1a1 | NA | 1.02E−41 | NA | NA | −0.402451 |
| Ddt | NA | 1.48E−41 | 1.33E−60 | NA | NA |
| Cox6a1 | 0.29212877 | 1.69E−41 | 9.18E−60 | 1.78E−87 | NA |
| Atp5j2 | NA | 3.32E−40 | NA | NA | NA |
| Ndufb2 | 0.2515952 | 5.29E−40 | 2.52E−28 | 1.66E−45 | NA |
| Ndufb9 | NA | 5.48E−40 | NA | NA | NA |
| Atp5h | 0.43416354 | 8.64E−40 | 7.16E−69 | 1.41E−124 | −0.351854 |
| Qdpr | NA | 1.45E−39 | NA | NA | NA |
| Atp5f1 | NA | 2.05E−39 | 1.01E−28 | NA | NA |
| Serpina1a | NA | 8.08E−39 | 4.32E−247 | NA | NA |
| Fabp2 | NA | 4.18E−38 | 3.19E−36 | NA | NA |
| Khk | 0.31010387 | 3.36E−37 | 4.03E−35 | 1.03E−67 | NA |
| Mpc2 | NA | 7.20E−37 | NA | NA | −0.268047 |
| Ndufb6 | NA | 4.30E−35 | 4.45E−28 | NA | −0.311203 |
| Elovl3 | −0.4426528 | 4.74E−35 | 6.30E−121 | 5.92E−91 | −0.252293 |
| Arl4d | NA | 6.71E−35 | NA | NA | −0.32852 |
| Akr1c14 | NA | 1.08E−34 | NA | NA | NA |
| Csad | NA | 1.25E−34 | 1.92E−72 | NA | NA |
| Ndufb11 | −0.275646 | 3.19E−34 | 8.28E−53 | 4.56E−22 | −0.367923 |
| Cox5b | NA | 4.68E−34 | 1.67E−32 | NA | NA |
| Rplp1 | NA | 4.91E−34 | 5.94E−23 | NA | NA |
| Fxyd1 | NA | 2.89E−33 | 3.83E−34 | NA | NA |
| Atp5j | NA | 3.59E−33 | NA | NA | NA |
| Rpl32 | −0.2846892 | 1.10E−32 | 2.90E−16 | 8.16E−20 | NA |
| Dio1 | NA | 1.11E−32 | NA | NA | NA |
| Nit2 | NA | 4.49E−32 | NA | NA | NA |
| Gnmt | NA | 1.40E−31 | 1.33E−133 | NA | NA |
| Ssr4 | NA | 1.87E−31 | NA | NA | NA |
| Ndufs6 | NA | 2.00E−31 | 4.72E−31 | NA | NA |
| Etfb | NA | 4.01E−31 | NA | NA | NA |
| Rsp14 | −0.3484055 | 1.50E−30 | 3.26E−44 | 1.07E−73 | −0.346341 |
| Blvrb | NA | 6.23E−30 | NA | NA | NA |
| Uqcr10 | NA | 2.60E−29 | NA | NA | NA |
| Cyp3a25 | 0.31857327 | 1.37E−27 | NA | 1.19E−134 | −0.410512 |
| Mup8 | −0.795757 | 2.07E−27 | 4.14E−25 | 1.23E−114 | −0.793731 |
| Sc5d | NA | 2.09E−27 | 3.57E−48 | NA | NA |
| Adipor2 | NA | 3.14E−27 | NA | NA | NA |
| Rpl36a1 | NA | 1.26E−26 | NA | NA | NA |
| Serpina1e | −0.6620459 | 1.97E−26 | 8.67E−65 | 0 | NA |
| Serpina1d | NA | 2.07E−26 | 1.74E−184 | NA | NA |
| Dcxr | NA | 2.61E−26 | 2.61E−43 | NA | −0.27134 |
| Cmbl | NA | 2.71E−26 | 1.48E−28 | NA | NA |
| Ndufb4 | NA | 2.93E−26 | 3.64E−28 | NA | NA |
| B2m | NA | 5.62E−26 | 1.86E−102 | NA | NA |
| Cyp4a12a | −0.2770512 | 6.69E−26 | NA | 7.00E−24 | −0.259776 |
| Hspe1 | −0.2813189 | 9.37E−26 | 1.53E−19 | 1.46E−15 | NA |
| Uqcrh | NA | 1.41E−25 | NA | NA | NA |
| mt-Co3 | −0.9151085 | 1.72E−25 | 9.57E−168 | 4.34E−176 | −1.233962 |
| Minos1 | NA | 2.00E−25 | NA | NA | NA |
| Mup14 | −0.7607669 | 2.00E−25 | NA | 2.64E−96 | −0.34089 |
| Cfhr2 | −0.3063257 | 4.83E−25 | 4.87E−29 | 2.09E−34 | NA |
| Timm13 | NA | 2.46E−24 | NA | NA | NA |
| 1500017E21Rik | NA | 2.47E−24 | NA | NA | NA |
| Hint2 | NA | 5.24E−24 | 6.90E−37 | NA | −0.29931 |
| Pbld1 | 0.3226882 | 7.81E−24 | NA | 4.53E−92 | NA |
| Glo1 | NA | 1.87E−23 | NA | NA | NA |
| Rpl13a | NA | 3.58E−22 | NA | NA | −0.314606 |
| Ces3b | −0.2632928 | 6.63E−22 | 1.58E−25 | 5.30E−27 | NA |
| Rps18 | NA | 8.18E−22 | NA | NA | −0.269207 |
| Serpina12 | −0.324744 | 1.67E−21 | NA | 2.90E−24 | NA |
| Ndufa2 | NA | 2.46E−21 | NA | NA | NA |
| Mup1 | −0.5568369 | 2.63E−21 | 1 | 7.40E−25 | −0.490972 |
| Bphl | NA | 5.02E−21 | NA | NA | NA |
| Prdx4 | −0.2519751 | 1.27E−20 | 6.96E−24 | 8.80E−10 | NA |
| Cyp2c54 | 0.25038702 | 1.78E−20 | 3.25E−32 | 9.37E−37 | −0.71286 |
| Rps2 | −0.3742466 | 2.13E−20 | 6.76E−40 | 5.09E−44 | −0.442508 |
| Acsl1 | 0.25633542 | 2.16E−20 | 1.40E−54 | 1.12E−70 | NA |
| Cebpa | NA | 3.89E−20 | NA | NA | −0.260692 |
| Uqcrb | NA | 6.59E−20 | NA | NA | NA |
| 1110008F13Rik | NA | 2.46E−19 | 4.13E−58 | NA | NA |
| Tmem243 | NA | 2.52E−19 | 2.35E−20 | NA | NA |
| Atp5g3 | NA | 4.43E−19 | NA | NA | NA |
| Rps15 | NA | 4.49E−19 | 7.79E−24 | NA | NA |
| Nme1 | −0.2857051 | 5.43E−19 | NA | 4.26E−14 | NA |
| Timm8b | NA | 8.39E−19 | NA | NA | NA |
| Ndufc2 | NA | 1.31E−18 | 1.02E−18 | NA | NA |
| S100a1 | NA | 1.68E−18 | 1.76E−35 | NA | NA |
| Sdhd | 0.32084192 | 2.07E−18 | NA | 8.20E−61 | NA |
| Usmg5 | 0.27873092 | 2.85E−18 | NA | 4.55E−56 | NA |
| Cyp27a1 | NA | 3.99E−18 | 4.44E−19 | NA | NA |
| Rps3 | NA | 5.25E−18 | NA | NA | NA |
| Slc22a30 | −0.2954008 | 5.26E−18 | NA | 1.34E−16 | NA |
| Rps9 | NA | 1.65E−17 | 1.94E−22 | NA | NA |
| Oaz1 | −0.4185288 | 1.73E−17 | 8.14E−43 | 1.75E−59 | −0.339005 |
| Ndufb8 | NA | 3.81E−17 | NA | NA | NA |
| Hist1h1c | NA | 1.17E−16 | NA | NA | −0.331046 |
| Ces1f | NA | 1.45E−16 | NA | NA | NA |
| Slco1a1 | −0.2956402 | 2.16E−16 | NA | 1.36E−29 | NA |
| Ugt2a3 | NA | 3.75E−16 | 8.21E−34 | NA | NA |
| Psmb1 | NA | 3.95E−16 | NA | NA | NA |
| Baat | NA | 4.31E−16 | NA | NA | NA |
| Bckdha | NA | 6.82E−16 | NA | NA | NA |
| Atp5o | NA | 9.93E−16 | NA | NA | NA |
| C8b | −0.3601712 | 1.33E−15 | 1.74E−50 | 1.12E−72 | NA |
| Mrps18c | NA | 1.60E−15 | NA | NA | NA |
| Sdhb | NA | 2.09E−15 | NA | NA | NA |
| Aox3 | NA | 2.16E−15 | 2.60E−25 | NA | −0.275389 |
| Mdh1 | NA | 3.99E−15 | NA | NA | NA |
| Otc | NA | 6.99E−15 | NA | NA | NA |
| Sc4mol | NA | 7.58E−15 | 6.26E−23 | NA | NA |
| Igfbp2 | NA | 1.25E−14 | 4.02E−44 | NA | −0.348404 |
| Rps21 | NA | 2.02E−14 | NA | NA | NA |
| Sar1b | NA | 2.07E−14 | 1.79E−35 | NA | NA |
| Slc2a2 | 0.27229797 | 2.36E−14 | NA | 5.54E−60 | NA |
| Txn1 | NA | 2.50E−14 | NA | NA | NA |
| 0610011F06Rik | NA | 2.80E−14 | NA | NA | NA |
| Slc10a1 | 0.31432581 | 3.37E−14 | NA | 1.24E−84 | NA |
| Rps10 | NA | 4.59E−14 | NA | NA | NA |
| Esd | NA | 1.14E−13 | 9.90E−24 | NA | −0.302906 |
| Cyp4f14 | NA | 1.60E−13 | NA | NA | NA |
| Dhrs4 | NA | 9.59E−13 | NA | NA | NA |
| Mup2 | −0.5530585 | 9.59E−13 | 1 | 8.16E−38 | −0.650107 |
| Selk | −0.2893006 | 1.79E−12 | NA | 1.30E−13 | NA |
| Rpl14 | NA | 7.87E−12 | NA | NA | −0.261157 |
| Ndufs4 | NA | 1.01E−11 | NA | NA | NA |
| Abcd3 | NA | 1.44E−11 | NA | NA | NA |
| Cela1 | NA | 1.78E−11 | NA | NA | NA |
| Sparcl1 | −0.253617 | 2.56E−11 | 1.01E−31 | 3.40E−53 | −0.253617 |
| Aadat | NA | 3.04E−11 | NA | NA | NA |
| Agxt | NA | 4.28E−11 | NA | NA | NA |
| Sigmar1 | NA | 7.64E−11 | NA | NA | −0.282365 |
| Psma6 | NA | 8.48E−11 | NA | NA | NA |
| Cyp2c55 | NA | 9.36E−11 | NA | NA | −0.316565 |
| Proc | NA | 9.66E−11 | NA | NA | NA |
| Cebpb | NA | 9.79E−11 | 2.38E−50 | NA | NA |
| Rpl13 | NA | 1.55E−10 | 1.78E−21 | NA | −0.314606 |
| Ubb | −0.2644328 | 6.19E−10 | 2.58E−11 | 0.12064603 | −0.263774 |
| Rps24 | NA | 8.21E−10 | NA | NA | NA |
| Ppp2r5a | 0.25089771 | 9.42E−10 | NA | 7.14E−52 | −0.263514 |
| Lipc | NA | 1.09E−09 | NA | NA | NA |
| Rabac1 | NA | 1.45E−09 | NA | NA | NA |
| Mvd | NA | 1.91E−09 | NA | NA | −0.327327 |
| Zfp683 | NA | 5.25E−09 | 1.05E−25 | NA | −0.421689 |
| Park7 | 0.25151769 | 5.75E−09 | NA | 1.55E−56 | NA |
| Cyp2b10 | NA | 2.09E−08 | 9.40E−117 | NA | −1.254972 |
| Rpl34 | NA | 8.13E−08 | NA | NA | NA |
| mt-Atp6 | −0.3234264 | 1.61E−07 | 1.31E−25 | 3.18E−23 | −0.852556 |
| Sco2 | NA | 5.95E−07 | 1.32E−23 | NA | NA |
| Ndufa5 | NA | 1.96E−06 | NA | NA | NA |
| Serpina3c | −0.472511 | 7.83E−06 | 1.55E−34 | 8.19E−59 | −0.554657 |
| 4931406C07Rik | NA | 9.62E−06 | NA | NA | NA |
| Gstp1 | −0.8558314 | 0.00132565 | 1.39E−27 | 1.88E−150 | NA |
| Ftl1 | −0.2988085 | 0.34648571 | 2.43E−17 | 1 | −0.346327 |
| mt-Nd2 | −0.8980548 | NA | 0 | 0 | −1.128777 |
| mt-Nd5 | −0.7991472 | NA | 0 | 7.18E−267 | −1.145551 |
| mt-Co1 | −1.0433076 | NA | 0 | 0 | −1.381308 |
| mt-Cytb | −0.8545555 | NA | 0 | 0 | −1.296044 |
| mt-Rnr2 | −0.5594463 | NA | 5.04E−251 | 1.98E−232 | −0.873558 |
| Serpina1e | NA | NA | 7.17E−151 | NA | NA |
| mt-Rnr1 | −0.4853999 | NA | 2.01E−123 | 1.71E−195 | −0.872187 |
| Ahcy | NA | NA | 6.43E−120 | NA | −0.375628 |
| Serpina1b | NA | NA | 4.66E−97 | NA | NA |
| Ces1d | 0.28525464 | NA | 8.07E−91 | 7.83E−69 | −0.385155 |
| Cth | NA | NA | 1.34E−76 | NA | −0.308848 |
| Ech1 | 0.2504813 | NA | 5.62E−72 | 1.35E−56 | NA |
| Ephx2 | 0.28125856 | NA | 3.71E−68 | 3.91E−109 | NA |
| mt-Nd6 | NA | NA | 1.67E−57 | NA | −0.515107 |
| Cyp2d10 | NA | NA | 1.31E−55 | NA | NA |
| Clpx | NA | NA | 1.07E−46 | NA | −0.306093 |
| Gm4076 | −0.3083212 | NA | 1.37E−35 | 2.57E−39 | −0.342158 |
| Acot1 | 0.30784004 | 3.05E−10 | 5.23E−33 | 1.49E−100 | NA |
| Gm5096 | NA | NA | 1.95E−32 | NA | −0.399548 |
| Gas5 | NA | NA | 2.74E−31 | NA | NA |
| Mup20 | −0.4762073 | NA | 9.36E−26 | 3.88E−128 | NA |
| Akr1c19 | NA | NA | 7.78E−21 | NA | NA |
| Eci1 | NA | NA | 2.48E−20 | NA | NA |
| Fcgrt | 0.25847884 | NA | 8.21E−19 | 4.12E−64 | NA |
| Gm20594 | −0.3565586 | 1.60E−05 | 2.58E−08 | 1.55E−09 | −0.559578 |
| Eef1b2 | −0.3173232 | NA | NA | 2.36E−38 | NA |
| Rps8 | −0.2746013 | NA | NA | 8.37E−12 | NA |
| C6 | −0.2669877 | NA | NA | 3.03E−10 | NA |

| | PH48logFC | PH120logFC | PH3p | PH48p | PH120p |
|---|---|---|---|---|---|
| Mup11 | −1.41509233 | −1.769207 | 0 | 0 | 0 |
| Mup19 | −3.63217051 | −3.348976 | 0 | 0 | 0 |
| Mup17 | −2.2696211 | −2.382124 | 0 | 0 | 0 |
| Fabp1 | −0.54216735 | −0.826492 | NA | 8.21E−214 | 0 |
| Akr1c6 | −0.50114352 | −0.457642 | 6.85E−150 | 1.04E−103 | 3.18E−123 |
| Mgst1 | −0.28624093 | −0.368754 | 2.50E−131 | 3.71E−58 | 2.20E−129 |
| Mup9 | −2.21914429 | −2.520659 | 0 | 0 | 0 |
| Apoc1 | −0.31073779 | NA | NA | 1.04E−90 | NA |
| Mup16 | −0.87654486 | −1.245137 | 1.76E−222 | 2.12E−149 | 0 |
| Adh1 | NA | NA | 3.27E−106 | NA | NA |
| Cyp2e1 | −0.29860874 | 0.2969 | 4.23E−36 | 2.45E−19 | 6.94E−06 |
| Gpx1 | −0.32198413 | NA | NA | 2.36E−120 | NA |
| Mup3 | −1.4237591 | −1.310718 | 0 | 0 | 0 |
| Mup18 | −2.37334874 | −2.585635 | 0 | 0 | 0 |
| Apoc4 | NA | −0.50176 | 2.38E−146 | NA | 1.19E−182 |
| Mup6 | −2.64537347 | −2.588052 | 0 | 0 | 0 |
| Nudt7 | −0.56604376 | NA | NA | 9.48E−52 | NA |
| Mup12 | −1.21051737 | −1.228078 | 2.77E−105 | 1.10E−243 | 0 |
| Car3 | −1.29219188 | −1.168631 | NA | 2.01E−301 | 0 |
| Apoa2 | −0.61598119 | −0.427739 | NA | 0 | 1.04E−276 |
| Selenbp2 | −0.8571994 | −0.354512 | NA | 9.70E−194 | 5.81E−73 |
| 2810007J24Rik | −1.09399829 | −0.337773 | NA | 2.63E−273 | 7.72E−74 |
| Uqcrq | −0.34186946 | −0.485355 | 1.28E−72 | 1.11E−51 | 2.42E−124 |
| Ttr | −0.28613839 | NA | NA | 3.87E−82 | NA |
| Cyp2c29 | NA | −0.997503 | 2.76E−235 | NA | 3.62E−184 |
| Tmem205 | −0.36376261 | NA | NA | 6.01E−57 | NA |
| Gsta3 | −0.57982304 | −0.796927 | 2.50E−189 | 2.50E−118 | 3.08E−250 |
| Cyb5 | −0.48647684 | −0.751689 | 0 | 1.24E−108 | 2.13E−288 |
| Hsd3b7 | −0.58164214 | NA | 3.52E−87 | 5.15E−93 | NA |
| Acaa1b | −0.74247809 | −0.855527 | 2.20E−79 | 8.89E−175 | 0 |
| Gm13775 | −1.14521098 | −1.171528 | 0 | 3.97E−216 | 5.37E−288 |
| Psen2 | NA | NA | 1.69E−77 | NA | NA |
| Mup15 | −1.3641826 | −1.359329 | 5.07E−136 | 2.34E−202 | 5.96E−268 |
| Cyp8b1 | −0.56548352 | −0.59929 | 3.21E−55 | 1.09E−90 | 1.54E−120 |
| Rps27l | −0.37756614 | −0.645823 | 1.30E−60 | 7.98E−50 | 1.42E−139 |
| Cox6c | −0.51741711 | −0.610223 | 1.20E−74 | 4.59E−79 | 2.30E−132 |
| Ugt2b5 | −0.68326299 | −0.716337 | 8.09E−193 | 1.47E−190 | 5.77E−289 |
| Hsd3b5 | −0.88646345 | −0.50626 | 1.00E−67 | 3.58E−129 | 4.13E−58 |
| Cyp2a5 | −0.52568008 | −0.869525 | 8.06E−111 | 1.48E−38 | 1.93E−69 |
| Ugt2b1 | −0.72210229 | −0.261758 | NA | 5.69E−140 | 1.59E−33 |
| Cyp1a2 | NA | −0.284567 | 3.86E−66 | NA | 8.09E−18 |
| Hgd | −0.2739982 | NA | NA | 2.46E−30 | NA |
| Adck3 | −0.56969289 | NA | 4.38E−91 | 6.36E−82 | NA |
| Mup21 | −1.34664515 | −1.637812 | 6.47E−176 | 1.32E−290 | 0 |
| Cyp2d9 | −0.61667658 | −0.325039 | NA | 8.72E−151 | 1.56E−76 |
| Apoe | −0.61588295 | NA | 3.58E−191 | 0 | NA |
| mt-Nd4 | −1.67314023 | −1.953206 | 0 | 0 | 0 |
| C8g | −0.45563856 | NA | NA | 2.41E−80 | NA |
| Mup7 | −0.84772673 | −0.844128 | 0.067179 | 2.63E−114 | 8.04E−142 |
| Ubc | NA | NA | 3.58E−41 | NA | NA |
| Pon1 | −0.42825679 | −0.326048 | 2.10E−60 | 1.97E−64 | 2.43E−57 |
| Oaf | NA | NA | 3.11E−47 | NA | NA |
| Ugt2b36 | −0.62967111 | −0.355796 | 5.82E−81 | 1.10E−145 | 4.78E−87 |
| Ces3a | −0.72100816 | NA | NA | 1.41E−206 | NA |
| Mup5 | −1.76898765 | −1.77461 | 0 | 2.08E−280 | 0 |
| Ndufa4 | −0.4035957 | −0.512723 | NA | 1.02E−52 | 4.13E−107 |
| Hagh | −0.28843067 | −0.398106 | NA | 2.99E−32 | 4.45E−73 |
| Cyp3a11 | NA | −0.803553 | 8.18E−135 | NA | 0 |
| Hsd3b3 | −0.34746055 | NA | NA | 3.82E−42 | NA |
| Hint1 | NA | −0.487016 | NA | NA | 9.22E−101 |
| Scp2 | −0.31951947 | NA | 5.16E−159 | 1.15E−50 | NA |
| Cox4i1 | −0.57523009 | −0.518813 | 5.97E−88 | 2.44E−141 | 2.23E−168 |
| Sod1 | −0.27785367 | −0.483374 | NA | 1.68E−63 | 3.17E−216 |
| Hacl1 | NA | −0.294917 | 4.71E−52 | NA | 2.73E−36 |
| Hpd | −0.31794334 | NA | NA | 2.00E−54 | NA |
| mt-Co2 | −1.41732822 | −1.394736 | 0 | 5.51E−209 | 1.21E−274 |
| Cyp2c37 | −0.41293867 | −0.820386 | 4.16E−114 | 1.60E−40 | 2.10E−130 |
| Inmt | −0.50241447 | −0.67979 | 9.17E−37 | 1.61E−65 | 1.56E−139 |
| Uox | −0.48042234 | NA | NA | 1.42E−89 | NA |
| Uqcr11 | NA | −0.368071 | NA | NA | 8.58E−61 |
| Ankrd55 | −1.95094236 | −1.470506 | 6.28E−208 | 2.52E−247 | 4.09E−185 |
| Prdx1 | NA | −0.353764 | NA | NA | 2.27E−73 |
| Hebp1 | −0.29746423 | −0.274169 | 1.26E−40 | 8.29E−32 | 3.96E−26 |
| Cox7b | −0.38652422 | −0.650086 | 6.26E−37 | 1.05E−43 | 3.36E−144 |
| mt-Nd1 | −1.15463044 | −1.543996 | 0 | 0 | 0 |
| Cox7a2 | NA | −0.441102 | NA | NA | 3.27E−59 |
| Chchd2 | −0.36975589 | −0.518624 | NA | 1.70E−48 | 1.10E−119 |
| Rarres2 | −0.49201391 | NA | NA | 1.76E−89 | NA |
| Ndufa6 | −0.33096547 | −0.418445 | 8.39E−24 | 3.11E−32 | 3.80E−59 |
| Rdh7 | −0.49691067 | NA | NA | 5.58E−132 | NA |
| Pah | −0.37985387 | NA | NA | 8.65E−60 | NA |
| Leap2 | NA | −0.404793 | NA | NA | 1.27E−50 |
| Cyp7b1 | −0.43815806 | NA | NA | 1.37E−54 | NA |
| Cyp2c50 | −0.46729844 | −0.51653 | 8.52E−186 | 1.54E−52 | 2.38E−78 |
| Mup4 | −1.94564614 | −1.909428 | 0 | 0 | 0 |
| Cml1 | −0.47269345 | NA | 2.15E−27 | 5.55E−63 | NA |
| Aadac | −0.74734282 | −0.466443 | NA | 6.76E−122 | 6.31E−69 |
| Mup13 | −0.87097239 | −0.875079 | 4.35E−116 | 1.89E−127 | 6.29E−165 |
| Ttc39c | −0.28961231 | NA | NA | 2.34E−27 | NA |
| Aldh1a1 | NA | NA | 1.39E−102 | NA | NA |
| Ddt | NA | −0.345839 | NA | NA | 1.40E−46 |
| Cox6a1 | −0.39061791 | −0.275277 | NA | 1.67E−61 | 4.29E−41 |
| Atp5j2 | −0.25505171 | −0.409577 | NA | 2.55E−25 | 3.23E−66 |
| Ndufb2 | NA | −0.336856 | NA | NA | 1.21E−30 |
| Ndufb9 | −0.29191091 | −0.483703 | NA | 2.15E−38 | 1.07E−104 |
| Atp5h | −0.35236298 | −0.555499 | 4.84E−19 | 7.17E−29 | 7.99E−68 |
| Qdpr | −0.31659518 | NA | NA | 1.80E−33 | NA |
| Atp5f1 | −0.46070987 | −0.428497 | NA | 1.11E−68 | 1.10E−77 |
| Serpina1a | −0.35536875 | −0.295263 | NA | 3.38E−75 | 6.23E−81 |
| Fabp2 | −0.48853359 | −0.527187 | NA | 5.45E−64 | 2.63E−76 |
| Khk | −0.53015038 | NA | NA | 8.89E−91 | NA |
| Mpc2 | NA | −0.39521 | 4.77E−43 | NA | 2.71E−68 |
| Ndufb6 | −0.47916274 | −0.518385 | 1.04E−35 | 4.11E−68 | 3.96E−85 |
| Elovl3 | −0.8122507 | −0.924846 | 2.20E−32 | 1.80E−150 | 1.57E−229 |
| Arl4d | −0.28894161 | NA | 5.36E−30 | 3.05E−26 | NA |
| Akr1c14 | NA | −0.280169 | NA | NA | 1.20E−21 |
| Csad | −0.30870307 | −1.214863 | NA | 4.35E−30 | 5.27E−267 |
| Ndufb11 | −0.39861879 | −0.479045 | 4.92E−46 | 7.18E−49 | 2.10E−70 |
| Cox5b | −0.39369185 | −0.373464 | NA | 3.39E−45 | 5.23E−43 |
| Rplp1 | NA | −0.274821 | NA | NA | 6.89E−20 |
| Fxyd1 | −0.27342229 | NA | NA | 2.95E−25 | NA |
| Atp5j | −0.30264483 | −0.415381 | NA | 4.96E−37 | 7.16E−66 |
| Rpl32 | NA | −0.498593 | NA | NA | 4.42E−73 |
| Dio1 | −0.46973235 | NA | NA | 9.15E−59 | NA |
| Nit2 | −0.25532127 | NA | NA | 6.79E−25 | NA |
| Gnmt | NA | −0.288175 | NA | NA | 8.65E−84 |
| Ssr4 | NA | −0.283161 | NA | NA | 7.71E−23 |
| Ndufs6 | NA | −0.341035 | NA | NA | 2.06E−29 |
| Etfb | NA | −0.270014 | NA | NA | 4.81E−54 |
| Rsp14 | NA | −0.607508 | 1.86E−83 | NA | 3.76E−152 |
| Blvrb | NA | −0.351075 | NA | NA | 2.20E−30 |
| Uqcr10 | NA | −0.382322 | NA | NA | 1.93E−47 |
| Cyp3a25 | −0.34306526 | −0.416457 | 5.44E−129 | 2.32E−54 | 4.60E−99 |
| Mup8 | −0.82995644 | −0.827182 | 9.31E−106 | 3.77E−91 | 3.80E−123 |
| Sc5d | NA | −0.274286 | NA | NA | 3.96E−19 |
| Adipor2 | −0.25255116 | NA | NA | 1.11E−22 | NA |
| Rpl36a1 | NA | −0.331845 | NA | NA | 3.44E−26 |
| Serpina1e | −0.3083608 | −0.725197 | NA | 1.15E−71 | 0 |
| Serpina1d | −0.58685276 | NA | NA | 5.86E−199 | NA |
| Dcxr | NA | NA | 1.59E−18 | NA | NA |
| Cmbl | −0.26536599 | NA | NA | 9.66E−25 | NA |
| Ndufb4 | NA | −0.331688 | NA | NA | 2.14E−33 |
| B2m | −0.43085129 | NA | NA | 2.11E−97 | NA |
| Cyp4a12a | −0.4760363 | NA | 1.18E−52 | 1.00E−65 | NA |
| Hspe1 | NA | −0.25791 | NA | NA | 3.13E−12 |
| Uqcrh | −0.29025996 | −0.483114 | NA | 1.52E−28 | 5.89E−83 |
| mt-Co3 | −1.21238839 | −1.229639 | 1.47E−287 | 9.73E−175 | 3.03E−229 |
| Minos1 | NA | −0.308404 | NA | NA | 8.00E−23 |
| Mup14 | −0.49742727 | −0.741009 | 1 | 2.38E−27 | 8.52E−68 |
| Cfhr2 | −0.63061767 | −0.42885 | NA | 7.59E−98 | 1.02E−63 |
| Timm13 | NA | −0.266268 | NA | NA | 1.44E−18 |
| 1500017E21Rik | −0.51793022 | NA | NA | 1.93E−69 | NA |
| Hint2 | −0.37377357 | −0.333604 | 6.95E−27 | 2.60E−44 | 3.41E−32 |
| Pbld1 | −0.26462773 | NA | NA | 2.23E−23 | NA |
| Glo1 | −0.37923349 | −0.317339 | NA | 3.51E−47 | 1.59E−34 |
| Rpl13a | NA | −0.480174 | 7.45E−64 | NA | 2.24E−99 |
| Ces3b | −0.81955869 | NA | NA | 6.86E−161 | NA |
| Rps18 | NA | −0.415076 | 5.39E−06 | NA | 1.19E−34 |
| Serpina12 | −0.61236747 | −0.404557 | NA | 7.52E−89 | 1.92E−45 |
| Ndufa2 | NA | −0.308306 | NA | NA | 9.67E−23 |
| Mup1 | −0.62326727 | −0.882907 | 0.022977 | 1.11E−33 | 1.78E−91 |
| Bphl | −0.29947816 | NA | NA | 1.70E−33 | NA |
| Prdx4 | −0.37802456 | −0.339425 | NA | 3.39E−40 | 1.74E−28 |
| Cyp2c54 | −0.54296022 | −0.632023 | 1.75E−163 | 2.37E−77 | 1.42E−114 |
| Rps2 | −0.26932139 | −0.47452 | 4.24E−69 | 2.20E−20 | 3.26E−67 |
| Acsl1 | −0.56803038 | −0.41703 | NA | 3.63E−104 | 1.27E−85 |
| Cebpa | −0.48161118 | NA | 6.82E−18 | 2.92E−66 | NA |
| Uqcrb | −0.33950492 | −0.359657 | NA | 1.34E−31 | 6.07E−33 |
| 1110008F13Rik | −0.28445851 | −0.327483 | NA | 3.77E−30 | 1.42E−34 |
| Tmem243 | NA | −0.275109 | NA | NA | 2.06E−22 |
| Atp5g3 | −0.30227788 | −0.357248 | NA | 2.31E−44 | 1.87E−78 |
| Rps15 | NA | −0.324543 | NA | NA | 3.43E−16 |
| Nme1 | NA | −0.291609 | NA | NA | 9.48E−21 |
| Timm8b | NA | −0.250989 | NA | NA | 2.42E−15 |
| Ndufc2 | −0.3708899 | −0.371343 | NA | 1.56E−33 | 9.91E−31 |
| S100a1 | −0.35554448 | NA | NA | 2.21E−35 | NA |
| Sdhd | NA | −0.302303 | NA | NA | 2.47E−32 |
| Usmg5 | NA | −0.283655 | NA | NA | 1.10E−10 |
| Cyp27a1 | −0.32622809 | NA | NA | 1.46E−36 | NA |
| Rps3 | NA | −0.339904 | NA | NA | 7.09E−57 |
| Slc22a30 | −0.33187075 | −0.257369 | NA | 7.77E−38 | 2.47E−15 |
| Rps9 | NA | −0.30861 | NA | NA | 6.38E−21 |
| Oaz1 | −0.42628615 | −0.327014 | 2.14E−37 | 1.27E−51 | 1.67E−31 |
| Ndufb8 | NA | −0.389897 | NA | NA | 1.46E−55 |
| Hist1h1c | NA | NA | 5.78E−33 | NA | NA |
| Ces1f | −0.45903711 | −0.533639 | NA | 9.66E−67 | 9.25E−112 |
| Slco1a1 | −0.63247791 | −0.542408 | NA | 5.52E−101 | 6.62E−92 |
| Ugt2a3 | −0.54739922 | −0.457903 | NA | 2.33E−87 | 2.95E−72 |
| Psmb1 | NA | −0.271575 | NA | NA | 5.68E−21 |
| Baat | −0.4458331 | −0.265815 | NA | 1.01E−65 | 1.48E−26 |
| Bckdha | −0.4047029 | NA | NA | 2.27E−47 | NA |
| Atp5o | NA | −0.268645 | NA | NA | 2.25E−21 |
| C8b | −0.25108437 | NA | NA | 1.50E−25 | NA |
| Mrps18c | NA | −0.274271 | NA | NA | 1.08E−17 |
| Sdhb | −0.35736225 | NA | NA | 2.51E−46 | NA |
| Aox3 | −0.42946046 | NA | 1.72E−66 | 1.23E−58 | NA |
| Mdh1 | −0.3290258 | NA | NA | 2.04E−40 | NA |
| Otc | −0.58497243 | NA | NA | 1.50E−116 | NA |
| Sc4mol | NA | −0.519569 | NA | NA | 1.06E−80 |
| Igfbp2 | −0.5766821 | −0.717833 | 3.39E−70 | 8.96E−103 | 3.60E−190 |
| Rps21 | NA | −0.316866 | NA | NA | 2.81E−22 |
| Sar1b | −0.30189148 | −0.422088 | NA | 4.44E−36 | 6.58E−68 |
| Slc2a2 | −0.29126785 | NA | NA | 2.49E−31 | NA |
| Txn1 | NA | −0.277353 | NA | NA | 6.45E−28 |
| 0610011F06Rik | −0.28593883 | NA | NA | 3.51E−28 | NA |
| Slc10a1 | −0.39548808 | −0.273026 | NA | 8.15E−51 | 4.19E−30 |
| Rps10 | NA | −0.25266 | NA | NA | 3.85E−13 |
| Esd | −0.38153946 | −0.33891 | 9.71E−44 | 1.94E−49 | 5.03E−45 |
| Cyp4f14 | −0.28356503 | NA | NA | 6.47E−28 | NA |
| Dhrs4 | NA | −0.268863 | NA | NA | 5.96E−15 |
| Mup2 | −0.73872198 | −0.724059 | 4.29E−84 | 2.14E−98 | 5.33E−115 |
| Selk | NA | −0.279249 | NA | NA | 4.58E−18 |
| Rpl14 | NA | −0.357061 | 2.50E−13 | NA | 2.16E−37 |
| Ndufs4 | NA | −0.29996 | NA | NA | 2.26E−18 |
| Abcd3 | −0.32683847 | NA | NA | 1.45E−40 | NA |
| Cela1 | −0.2526112 | NA | NA | 1.03E−30 | NA |
| Sparcl1 | −0.25361699 | −0.253617 | 3.78E−57 | 5.97E−29 | 2.01E−42 |
| Aadat | −0.32573171 | NA | NA | 9.13E−34 | NA |
| Agxt | −0.30382793 | NA | NA | 1.79E−39 | NA |
| Sigmar1 | NA | NA | 5.67E−17 | NA | NA |
| Psma6 | NA | −0.28171 | NA | NA | 2.82E−20 |
| Cyp2c55 | NA | −0.347841 | 6.52E−37 | NA | 2.21E−52 |
| Proc | −0.31977688 | NA | NA | 1.66E−39 | NA |
| Cebpb | −0.3063305 | −0.26767 | NA | 1.73E−23 | 3.07E−10 |
| Rpl13 | NA | −0.282418 | 7.45E−64 | NA | 3.52E−17 |
| Ubb | NA | NA | 0.0138701 | NA | NA |
| Rps24 | NA | −0.386465 | NA | NA | 6.91E−41 |
| Ppp2r5a | −0.31509887 | −0.281896 | 3.57E−15 | 3.37E−36 | 3.25E−21 |
| Lipc | −0.42305546 | NA | NA | 3.19E−59 | NA |
| Rabac1 | −0.34206368 | −0.27537 | NA | 3.25E−38 | 1.41E−19 |
| Mvd | NA | NA | 2.22E−59 | NA | NA |
| Zfp683 | −0.42168911 | −0.421689 | 1.10E−47 | 7.40E−24 | 3.13E−35 |
| Park7 | −0.27092931 | NA | NA | 9.02E−25 | NA |
| Cyp2b10 | −0.99521902 | −1.247317 | 1.74E−217 | 1.18E−87 | 1.24E−168 |
| Rpl34 | NA | −0.264586 | NA | NA | 1.43E−12 |
| mt-Atp6 | −0.91463545 | −0.87589 | 7.98E−137 | 3.35E−117 | 3.13E−134 |
| Sco2 | −0.39595151 | −0.250105 | NA | 1.04E−49 | 3.07E−16 |
| Ndufa5 | NA | −0.31028 | NA | NA | 4.19E−22 |
| Serpina3c | −0.58992683 | −0.595568 | 8.44E−109 | 8.61E−89 | 4.68E−124 |
| 4931406C07Rik | NA | −0.281781 | NA | NA | 3.10E−19 |
| Gstp1 | NA | −0.671665 | NA | NA | 4.28E−90 |
| Ftl1 | −0.25915144 | −0.347807 | 3.34E−05 | 1.49E−07 | 4.66E−11 |
| mt-Nd2 | −1.18941087 | −1.350784 | 0 | 0 | 0 |
| mt-Nd5 | −1.3978975 | −1.496458 | 0 | 1.28E−300 | 0 |
| mt-Co1 | −1.3991104 | −1.56995 | 0 | 0 | 0 |
| mt-Cytb | −1.37831332 | −1.554027 | 0 | 0 | 0 |
| mt-Rnr2 | −0.51967923 | −0.967453 | 0 | 2.69E−141 | 0 |
| Serpina1e | −0.27983515 | NA | NA | 1.24E−63 | NA |
| mt-Rnr1 | NA | −0.844781 | 0 | NA | 0 |
| Ahcy | −0.45598557 | −0.490113 | 1.82E−89 | 7.46E−73 | 1.57E−115 |
| Serpina1b | −0.29448079 | NA | NA | 1.99E−74 | NA |
| Ces1d | −0.44528336 | −1.201503 | 2.67E−121 | 2.43E−81 | 0 |
| Cth | −0.33223411 | −0.33921 | 6.91E−47 | 4.36E−37 | 3.95E−48 |
| Ech1 | −0.4146748 | −0.504022 | NA | 2.57E−62 | 2.34E−114 |
| Ephx2 | −0.57080098 | −0.398908 | NA | 1.49E−121 | 1.19E−91 |
| mt-Nd6 | −0.56026873 | −0.685505 | 1.57E−69 | 1.14E−72 | 2.74E−110 |
| Cyp2d10 | −0.32533265 | −0.262377 | NA | 3.76E−49 | 9.23E−44 |
| Clpx | −0.41084084 | −0.250587 | 6.23E−36 | 1.11E−53 | 3.74E−21 |
| Gm4076 | −0.34582632 | −0.342317 | 7.98E−74 | 2.87E−41 | 7.57E−56 |
| Acot1 | NA | −0.299327 | NA | NA | 2.65E−38 |
| Gm5096 | −0.33670416 | −0.350535 | 4.99E−39 | 1.27E−33 | 9.38E−37 |
| Gas5 | −0.29841752 | NA | NA | 6.31E−32 | NA |
| Mup20 | −1.23781717 | −1.081531 | NA | 0 | 0 |
| Akr1c19 | NA | −0.382533 | NA | NA | 1.32E−46 |
| Eci1 | −0.29260681 | −0.313353 | NA | 4.47E−29 | 3.71E−30 |
| Fcgrt | −0.27094541 | NA | NA | 1.81E−26 | NA |
| Gm20594 | −0.57378119 | −0.496964 | 4.48E−60 | 1.21E−52 | 1.33E−34 |
| Eef1b2 | NA | −0.367328 | NA | NA | 1.90E−45 |
| Rps8 | NA | −0.279846 | NA | NA | 2.52E−16 |
| C6 | −0.25510201 | NA | NA | 2.04E−21 | NA |

TABLE 15b
Shared Upregulated Differentially Expressed Genes

| ID | avg_logFC_APAP | avg_log_FC_PH | p_va_adj_APAP | p_val_adj_PH | A6logFC | A24logFC |
|---|---|---|---|---|---|---|
| Mt2 | 2.06273693 | 2.6060431 | 1.9E−237 | 0 | 2.06273693 | NA |
| Mt1 | 2.387707773 | 2.61085499 | 3.51E−232 | 0 | 2.387707773 | 0.348193009 |
| Plin2 | 1.461295758 | 0.72046308 | 8.58E−163 | 0 | 1.461295758 | NA |
| Il1r1 | 1.244999819 | 0.99816813 | 1.47E−162 | 0 | 1.244999819 | NA |
| Actb | 1.059835055 | 0.45698144 | 6.06E−159 | 2.1478E−210 | 1.059835055 | 0.38446276 |
| Cdkn1a | 1.2152557 | 0.44932547 | 2.99E−152 | 7.064E−169 | 1.2152557 | NA |
| Klf6 | 0.732407935 | 0.3616937 | 1.5E−147 | 8.0005E−203 | 0.732407935 | NA |
| Txnrd1 | 1.443578942 | 0.31497985 | 1.43E−144 | 3.22341E−84 | 1.443578942 | 0.260866185 |
| Gm26924 | 1.419027502 | 1.44535576 | 0 | 0 | 1.16085221 | 1.419027502 |
| 2010003K11Rik | 0.588354735 | 0.37221997 | 2.61E−131 | 1.0574E−239 | 0.588354735 | NA |
| Krt18 | 1.257881489 | 0.52035408 | 3.97E−121 | 5.88314E−34 | 1.257881489 | NA |
| Fga | 0.828543313 | 1.03943259 | 0 | 0 | 0.69335023 | 0.828543313 |
| Mat1a | 1.000433202 | 0.623735 | 0 | 0 | 0.790765268 | NA |
| n-R5-8s1 | 1.252818829 | 1.2895985 | 0 | 0 | 0.766100662 | 1.252818829 |
| Lars2 | 1.711400221 | 1.41912906 | 1.38E−293 | 0 | 1.391744455 | 1.711400221 |
| Myh9 | 1.160233135 | 0.63103566 | 3.1E−107 | 2.8315E−141 | 1.160233135 | 0.6391137 |
| Fgg | 0.680530925 | 0.80932271 | 0 | 0 | 0.615329068 | 0.680530925 |
| Gm24601 | 0.984391691 | 0.92456827 | 2.17E−275 | 0 | 0.46836359 | 0.984391691 |
| Fgb | 0.744973762 | 0.73951913 | 0 | 0 | 0.646934246 | 0.744973762 |
| Gm15564 | 1.820580657 | 1.48588593 | 6.31E−292 | 0 | 1.443066465 | 1.820580657 |
| Krt8 | 1.195874806 | 0.25227676 | 1.04E−103 | 0.182829207 | 1.195874806 | NA |
| Clec4f | 1.108180782 | 0.48733692 | 2.17E−103 | 1.7326E−119 | 1.108180782 | NA |
| Apoa4 | 1.367998503 | 1.18686306 | 8.56E−101 | 3.0126E−137 | 1.367998503 | NA |
| Bcl2l1 | 0.775118224 | 0.28031533 | 1.02E−99 | 1.1685E−128 | 0.775118224 | NA |
| Serpina3m | 0.967441963 | 0.77905275 | 1.04E−130 | 0 | 0.967441963 | NA |
| Rcan1 | 0.707283787 | 0.50923987 | 1.24E−93 | 4.5798E−279 | 0.707283787 | NA |
| D10Wsu102e | 0.58780186 | 0.25602216 | 2.09E−90 | 1.0336E−141 | 0.58780186 | NA |
| Slc39a14 | 1.012749203 | 0.83158494 | 2.25E−81 | 0 | 1.012749203 | 0.323040019 |
| C1qb | 0.681679654 | 0.36130977 | 1.02E−79 | 6.3479E−100 | 0.681679654 | NA |
| Tmsb4x | 0.780616209 | 0.34765791 | 7.99E−79 | 1.30373E−70 | 0.780616209 | NA |
| Ctss | 0.764167083 | 0.27018313 | 2.26E−78 | 2.67839E−57 | 0.764167083 | NA |
| Mrap | 0.884099926 | 0.28097461 | 2.65E−73 | 1.03675E−10 | 0.884099926 | NA |
| Hnf4a | 0.879108718 | 0.64419525 | 3.56E−83 | 5.6829E−290 | 0.879108718 | 0.454905579 |
| Csf1r | 0.704458586 | 0.28468437 | 4.7E−70 | 3.78986E−37 | 0.704458586 | NA |
| Trp53inp1 | 1.008414088 | 0.2692784 | 1.52E−67 | 1.01347E−72 | 1.008414088 | 0.367960906 |
| Nop58 | 0.663449653 | 0.25756665 | 2.13E−65 | 1.11973E−93 | 0.663449653 | NA |
| Mfsd2a | 0.666142747 | 0.49635188 | 5.34E−233 | 1.6404E−219 | 0.666142747 | NA |
| Lyz2 | 0.732183369 | 0.40273572 | 2.49E−63 | 3.32908E−93 | 0.732183369 | NA |
| Bach1 | 0.832333098 | 0.53323737 | 2.51E−63 | 5.5373E−247 | 0.832333098 | NA |
| Fam134b | 0.893562894 | 0.86557818 | 5.02E−63 | 0 | 0.893562894 | NA |
| Cd5l | 0.693485826 | 0.49371428 | 1.08E−62 | 4.6843E−138 | 0.693485826 | NA |
| Serpina3n | 0.848313239 | 1.75975894 | 9.93E−85 | 0 | 0.848313239 | 0.413696164 |
| Apob | 0.580156235 | 0.50539758 | 4.36E−68 | 8.048E−169 | 0.580156235 | 0.4272457 |
| Eif1a | 0.795281298 | 0.29521995 | 1.8E−60 | 7.80345E−96 | 0.795281298 | NA |
| Epas1 | 0.775763153 | 0.38425941 | 7.57E−60 | 2.1215E−138 | 0.775763153 | NA |
| Sdc4 | 0.619597869 | 0.43560504 | 9.47E−59 | 2.7024E−193 | 0.619597869 | NA |
| C3 | 0.449521065 | 1.07195093 | 1.23E−125 | 0 | 0.44276078 | 0.449521065 |
| Wfdc17 | 0.486653465 | 0.31732989 | 2.57E−58 | 1.3239E−101 | 0.486653465 | NA |
| Fkbp5 | 0.516128063 | 0.66330484 | 1.44E−57 | 0 | 0.516128063 | NA |
| Btg2 | 0.832086378 | 0.77268683 | 3.81E−56 | 7.2437E−290 | 0.832086378 | NA |
| Tiparp | 0.545567123 | 0.48912215 | 8.73E−55 | 7.0657E−236 | 0.545567123 | NA |
| Cldn14 | 0.364540783 | 0.51158353 | 1.86E−52 | 4.316E−281 | 0.364540783 | NA |
| Abhd2 | 0.484366625 | 0.26724151 | 1.13E−50 | 1.29723E−66 | 0.484366625 | NA |
| Picalm | 0.754529703 | 0.27690217 | 1.39E−50 | 1.27269E−76 | 0.754529703 | NA |
| Igfbp1 | 1.058689301 | 1.2887899 | 1.68E−50 | 0 | 1.058689301 | 0.316195447 |
| Stat3 | 0.715400235 | 0.48390463 | 2.74E−50 | 3.5753E−174 | 0.715400235 | 0.297428438 |
| Myo1e | 0.614341001 | 0.39945667 | 9.34E−50 | 7.8258E−162 | 0.614341001 | NA |
| Tmem87b | 0.434399269 | 0.28833059 | 1.07E−49 | 8.7183E−155 | 0.434399269 | NA |
| Slc10a2 | 0.299006165 | 0.30563824 | 2.04E−49 | 6.538E−197 | 0.299006165 | NA |
| Lcn2 | 0.412194792 | 2.39758181 | 2.72E−49 | 0 | 0.412194792 | NA |
| Jmjd1c | 0.636619821 | 0.39612601 | 3.43E−49 | 1.0512E−144 | 0.636619821 | NA |
| Itih3 | 0.546240791 | 1.22302231 | 1.11E−89 | 0 | 0.546240791 | 0.492908293 |
| Tacc2 | 0.329332932 | 0.87762397 | 3.95E−42 | 0 | 0.329332932 | NA |
| Eef2 | 0.525855561 | 0.32170576 | 2.71E−41 | 1.70104E−97 | 0.525855561 | 0.270115373 |
| Ptpn1 | 0.41633121 | 0.26826323 | 3.51E−41 | 6.9212E−130 | 0.41633121 | NA |
| Tnfrsf1a | 0.599573483 | 0.34684503 | 3.86E−41 | 3.4204E−112 | 0.599573483 | NA |
| Ddx3x | 0.656890035 | 0.47100162 | 1.07E−40 | 1.3581E−155 | 0.656890035 | NA |
| Itih2 | 0.501091194 | 0.33671421 | 3.43E−38 | 1.5587E−122 | 0.501091194 | 0.298659728 |
| St3gal5 | 0.607639775 | 0.88276152 | 1.06E−170 | 0 | 0.607639775 | NA |
| Abca1 | 0.724477701 | 0.50506234 | 8.81E−38 | 3.1915E−172 | 0.724477701 | 0.439378423 |
| Bcl3 | 0.289017645 | 0.41965014 | 3.63E−37 | 3.0494E−251 | 0.289017645 | NA |
| Asl | 0.558638879 | 0.29767377 | 3.45E−117 | 1.76334E−83 | 0.558638879 | NA |
| Aldob | 0.365460648 | 0.49020085 | 9.03E−178 | 0 | 0.315339576 | NA |
| Ugdh | 0.633076584 | 0.26044353 | 5.17E−34 | 4.68355E−05 | 0.633076584 | NA |
| H3f3b | 0.702914634 | 0.75556711 | 7.03E−34 | 4.4441E−288 | 0.702914634 | NA |
| Xbp1 | 0.603165814 | 0.539986 | 9.59E−34 | 1.3385E−208 | 0.603165814 | NA |
| Ctsl | 0.530101805 | 0.25652087 | 1.93E−33 | 1.56147E−59 | 0.530101805 | NA |
| Hspb8 | 0.633933124 | 0.66784725 | 2E−33 | 9.1859E−267 | 0.633933124 | NA |
| Eif4g2 | 0.559692663 | 0.3843028 | 1.62E−67 | 2.505E−110 | 0.559692663 | 0.465990267 |
| Epb4.1 | 0.562789274 | 0.34943067 | 1.67E−30 | 1.9295E−113 | 0.562789274 | NA |
| Rnd3 | 0.36548808 | 0.28514768 | 3.93E−30 | 4.6581E−134 | 0.36548808 | NA |
| Ddx5 | 0.522944475 | 0.25167504 | 5.9E−30 | 3.02351E−75 | 0.522944475 | NA |
| Plec | 0.467512335 | 0.26712212 | 8.3E−30 | 2.03726E−37 | 0.467512335 | NA |
| Jak1 | 0.603850485 | 0.36276669 | 1.34E−29 | 2.4382E−103 | 0.603850485 | 0.254122657 |
| Errfi1 | 0.393680047 | 0.50085053 | 1.52E−29 | 7.4336E−262 | 0.393680047 | NA |
| Iigp1 | 0.768753667 | 0.30588909 | 7.59E−66 | 4.94888E−49 | 0.768753667 | 0.454178892 |
| Lrg1 | 0.490792829 | 1.26050974 | 1.37E−62 | 0 | 0.490792829 | 0.436838294 |
| Gpt2 | 0.539114072 | 0.53333178 | 2.49E−28 | 3.6697E−218 | 0.539114072 | NA |
| Gjb2 | 0.60822545 | 0.63950744 | 3.74E−38 | 9.9227E−255 | 0.60822545 | 0.391136188 |
| Dst | 0.510971252 | 0.32990555 | 1.75E−35 | 7.3975E−105 | 0.510971252 | 0.324301394 |
| Wbp1l | 0.553507326 | 0.40913816 | 4.08E−134 | 1.2925E−127 | 0.553507326 | NA |
| Fn1 | 0.699634526 | 1.01877291 | 1.87E−106 | 0 | 0.521230804 | 0.699634526 |
| Grn | 0.540768685 | 0.40331576 | 1.07E−154 | 3.4391E−128 | 0.540768685 | NA |
| Mpeg1 | 0.493478112 | 0.54117001 | 1.8E−24 | 4.2393E−174 | 0.493478112 | NA |
| Aff4 | 0.616309327 | 0.34418622 | 1.96E−24 | 7.00106E−97 | 0.616309327 | NA |
| Gm26917 | 0.823736053 | 0.47555416 | 5.26E−54 | 2.4164E−144 | 0.60120474 | 0.823736053 |
| Tns1 | 0.434939232 | 0.33981795 | 8.9E−24 | 4.5763E−128 | 0.434939232 | NA |
| Arg1 | 0.317948822 | 0.38161635 | 8.62E−23 | 2.227E−218 | 0.317948822 | NA |
| Arhgef12 | 0.590764816 | 0.30486583 | 8.83E−23 | 1.49183E−67 | 0.590764816 | 0.304679431 |
| Man2a1 | 0.497115325 | 0.36999936 | 2.32E−81 | 3.60298E−98 | 0.497115325 | 0.28327895 |
| Lpgat1 | 0.595219291 | 0.40078798 | 3.53E−63 | 1.2472E−107 | 0.595219291 | 0.394625955 |
| Il6ra | 0.304392668 | 0.25169684 | 5.08E−20 | 2.9323E−108 | 0.304392668 | NA |
| Ptp4a1 | 0.262691158 | 0.39313737 | 3.82E−19 | 6.0997E−190 | 0.262691158 | NA |
| Atp2a2 | 0.494962728 | 0.35036327 | 7.58E−69 | 8.21385E−94 | 0.494962728 | NA |
| Aass | 0.477517138 | 0.70561534 | 3.07E−18 | 1.0663E−286 | 0.477517138 | NA |
| Nedd4l | 0.281232817 | 0.2718271 | 4.9E−18 | 1.5124E−116 | 0.281232817 | NA |
| Egr1 | 0.584558888 | 0.62048047 | 2.8E−17 | 7.2676E−185 | 0.584558888 | NA |
| Litaf | 0.394366845 | 0.39600771 | 2.04E−16 | 2.337E−132 | 0.394366845 | NA |
| Plk3 | 0.416624571 | 0.48801637 | 3.12E−16 | 8.2222E−205 | 0.416624571 | NA |
| Slc41a2 | 0.278262888 | 0.44132177 | 2.13E−34 | 2.2246E−166 | 0.260152635 | 0.278262888 |
| Hsp90ab1 | 0.322748941 | 0.45585151 | 8.34E−16 | 1.16529E−94 | 0.322748941 | NA |
| Tgm2 | 0.399330712 | 0.37123494 | 1.43E−15 | 5.49556E−85 | 0.399330712 | NA |
| Shb | 0.315236194 | 0.43061356 | 4.68E−15 | 4.0274E−182 | 0.315236194 | NA |
| Tmed5 | 0.401633325 | 0.29601878 | 5.01E−102 | 1.362E−80 | 0.401633325 | NA |
| Atp1b1 | 0.370203015 | 0.42480123 | 3.27E−13 | 1.3155E−145 | 0.370203015 | NA |
| Ctsb | 0.521310448 | 0.55397449 | 4.83E−13 | 4.5123E−149 | 0.521310448 | NA |
| Mug2 | 0.370406564 | 0.53657702 | 6.96E−26 | 3.3989E−113 | 0.368295871 | 0.370406564 |
| Slc25a47 | 0.502661475 | 0.63592878 | 1.45E−158 | 0 | 0.428932953 | NA |
| Atf6 | 0.377819844 | 0.28052116 | 7.66E−12 | 1.54665E−82 | 0.377819844 | NA |
| Ell2 | 0.417241283 | 0.37174598 | 2.55E−22 | 9.305E−113 | 0.417241283 | 0.288090516 |
| Brd2 | 0.571515166 | 0.25844685 | 2.65E−12 | 8.83728E−51 | 0.571515166 | 0.266388486 |
| Snx10 | 0.285642594 | 0.55824463 | 2.35E−11 | 1.5948E−251 | 0.285642594 | NA |
| Steap4 | 0.475973731 | 1.89001517 | 6.59E−69 | 0 | 0.437231109 | 0.475973731 |
| Gfra1 | 0.386382804 | 0.47469443 | 2.65E−92 | 4.734E−168 | 0.386382804 | NA |
| Cp | 0.436243259 | 0.65551115 | 6.52E−192 | 5.8053E−290 | 0.254076812 | 0.317404056 |
| Slc25a51 | 0.263985158 | 0.37215766 | 6.14E−10 | 1.1132E−164 | 0.263985158 | NA |
| Eef1a1 | 0.439251983 | 0.34076819 | 2.2E−81 | 6.1414E−119 | 0.282586942 | 0.439251983 |
| Ivns1abp | 0.356610399 | 0.46254094 | 1.2E−09 | 9.4406E−168 | 0.356610399 | NA |
| Por | 0.335963246 | 0.29074557 | 1.06E−73 | 3.92838E−83 | 0.335963246 | 0.271635533 |
| Plin5 | 0.352087102 | 0.48925753 | 1.22E−125 | 1.0072E−205 | 0.296281512 | 0.277759837 |
| Ppip5k2 | 0.320939415 | 0.30299148 | 7.41E−09 | 3.6258E−115 | 0.320939415 | NA |
| Fmo5 | 0.286219883 | 0.6901608 | 4.71E−64 | 0 | 0.286219883 | NA |
| Ccnl1 | 0.256623048 | 0.34133845 | 1.44E−08 | 1.6662E−120 | 0.256623048 | NA |
| Nrp1 | 0.360791537 | 0.26879679 | 1.93E−28 | 7.49426E−79 | 0.360791537 | 0.334097753 |
| H2-D1 | 0.286522764 | 0.43318371 | 2.31E−08 | 1.1359E−105 | 0.286522764 | NA |
| Cxcl1 | 0.449766784 | 1.23039802 | 9.38E−68 | 0 | 0.370070418 | 0.449766784 |
| Hdlbp | 0.565147241 | 0.39403065 | 5.1E−95 | 2.52472E−71 | 0.345721467 | 0.565147241 |
| Hspa5 | 0.610549064 | 0.33976913 | 9.07E−184 | 1.02395E−98 | 0.268221373 | 0.610549064 |
| Atp1a1 | 0.419079339 | 0.26171891 | 3.57E−63 | 2.95645E−65 | 0.419079339 | 0.270031448 |
| Nucks1 | 0.355785584 | 1 | 2.82E−07 | 0.271055696 | 0.355785584 | NA |
| Ctnnd1 | 0.251848095 | 0.25284616 | 5.48E−07 | 1.41477E−35 | 0.251848095 | NA |
| Sult1a1 | 0.560453494 | 0.37102354 | 9.07E−216 | 2.6725E−99 | 0.318989978 | NA |
| H6pd | 0.343718187 | 0.49196579 | 2.22E−16 | 1.0068E−111 | 0.343718187 | 0.261357904 |
| Serinc3 | 0.35928911 | 0.29396497 | 4.67E−75 | 2.70765E−85 | 0.35928911 | NA |
| Il6st | 0.333486533 | 0.38288189 | 3.02E−29 | 3.02061E−73 | 0.316786986 | 0.333486533 |
| Elovl5 | 0.335236661 | 0.38226719 | 0.00000254 | 1.0806E−102 | 0.335236661 | NA |
| Cyp3a13 | 0.284411943 | 0.42068008 | 0.00000493 | 6.40777E−81 | 0.284411943 | NA |
| Rmnd5a | 0.318903459 | 0.27347302 | 0.0000109 | 5.48218E−73 | 0.318903459 | NA |
| mmu-mir-6236 | 0.787474192 | 1.25724839 | 7.24E−35 | 5.137E−284 | 0.520569164 | 0.787474192 |
| Saa2 | 0.964488054 | 4.43818759 | 8.96E−95 | 0 | 0.558658405 | 0.964488054 |
| Cpt1a | 0.372935212 | 0.42860734 | 2.13E−98 | 3.3126E−139 | 0.261119591 | NA |
| Acsl4 | 0.314743242 | 0.27327587 | 1.14E−25 | 5.43114E−70 | 0.298529302 | 0.314743242 |
| Trib1 | 0.331693479 | 0.58175428 | 7.49E−21 | 1.5671E−204 | 0.331693479 | 0.287458438 |
| Psap | 0.360973931 | 0.31847224 | 0.25780026 | 5.29626E−54 | 0.257800256 | NA |
| Apcs | 0.75840106 | 2.04948541 | 2.16E−78 | 0 | 0.298925674 | 0.606730845 |
| Gm23935 | 1 | 1.20724431 | 3.7E−142 | 1.3842E−297 | 0.424222263 | 0.721300311 |
| Sephs2 | 0.889992859 | 0.618707 | 2.83E−263 | 1.0636E−281 | NA | 0.889992859 |
| Fgl1 | 0.864933885 | 1.56188077 | 1.6E−238 | 0 | NA | 0.864933885 |
| Hyou1 | 0.846484822 | 0.49615608 | 1.62E−156 | 3.5998E−141 | NA | 0.846484822 |
| Calr | 0.555145472 | 0.39195553 | 1.67E−148 | 1.6119E−116 | NA | 0.555145472 |
| Scd1 | 0.726687286 | 1.081677 | 2.08E−139 | 0 | 0.466125013 | 0.726687286 |
| Itih4 | 0.525074867 | 1.18011817 | 4.26E−137 | 0 | NA | 0.525074867 |
| Hp | 0.431328341 | 1.5848899 | 8.95E−137 | 0 | NA | 0.431328341 |
| Pdia3 | 0.593769188 | 0.27615686 | 7.36E−136 | 3.3607E−53 | NA | 0.593769188 |
| P4hb | 0.405223744 | 0.38506968 | 2.27E−113 | 1.31E−132 | NA | 0.405223744 |
| Glud1 | 0.451048591 | 0.35909971 | 6.55E−110 | 1.1097E−139 | NA | 0.451048591 |
| Pzp | 0.41600896 | 0.63685181 | 3.34E−97 | 0 | NA | 0.41600896 |
| Serpina10 | 0.565046404 | 0.87104418 | 8.21E−94 | 0 | NA | 0.565046404 |
| Creld2 | 0.608652695 | 0.36247761 | 1.18E−91 | 5.90495E−44 | NA | 0.608652695 |
| Egfr | 0.504957782 | 0.65440823 | 1.72E−79 | 5.4692E−228 | NA | 0.504957782 |
| Orm1 | 0.365144434 | 1.82376077 | 4.87E−78 | 0 | NA | 0.365144434 |
| Kng1 | 0.265777676 | 0.54683549 | 1.32E−71 | 0 | NA | 0.265777676 |
| Elovl2 | 0.45224665 | 0.28503861 | 1.22E−62 | 2.60256E−69 | NA | 0.45224665 |
| Cps1 | 0.32450205 | 0.33642945 | 1.45E−55 | 1.1876E−145 | NA | 0.32450205 |
| Prg4 | 0.430643187 | 0.81083567 | 3.2E−54 | 8.3602E−244 | NA | 0.430643187 |
| Manf | 0.468881688 | 0.28295063 | 1.6E−53 | 0.023268676 | NA | 0.468881688 |
| Orm2 | 0.271460545 | 2.40508674 | 2.71E−53 | 0 | NA | 0.271460545 |
| Cfh | 0.400919606 | 0.83288399 | 3.24E−52 | 0 | NA | 0.400919606 |
| Hc | 0.367935082 | 0.61123296 | 1.11E−51 | 2.3088E−207 | NA | 0.367935082 |
| Dhcr24 | 0.423756767 | 0.75973058 | 3.03E−107 | 1.4499E−130 | NA | 0.423756767 |
| Cpb2 | 0.322179327 | 0.4037904 | 5.47E−45 | 3.3355E−100 | 0.321495225 | 0.322179327 |
| Plg | 0.261736006 | 0.5965244 | 1.34E−44 | 0 | NA | 0.261736006 |
| Acox1 | 0.275171428 | 0.54002216 | 1.78E−40 | 0 | NA | 0.275171428 |
| Dnajc12 | 0.322094554 | 0.31290759 | 1.14E−39 | 2.5383E−30 | NA | 0.322094554 |
| C8a | 0.333650228 | 0.40419805 | 7.61E−55 | 2.99834E−96 | NA | 0.333650228 |
| Qsox1 | 0.342352868 | 0.72506211 | 9.2E−39 | 2.2371E−241 | NA | 0.342352868 |
| Slc3a1 | 0.340997565 | 0.38179133 | 7.1E−38 | 1.93288E−94 | NA | 0.340997565 |
| Fos | 0.432003299 | 0.52473333 | 1.43E−36 | 3.547E−125 | NA | 0.432003299 |
| Dstn | 0.324301394 | 0.27138172 | 1.75E−35 | 2.89799E−82 | NA | 0.324301394 |
| Cyb5r3 | 0.339033056 | 0.33768468 | 3.1E−34 | 1.89897E−92 | NA | 0.339033056 |
| Efna1 | 0.336097237 | 0.26845518 | 1.73E−31 | 2.54995E−60 | NA | 0.336097237 |
| Slc7a2 | 0.352564931 | 0.62232653 | 3.47E−68 | 6.7383E−265 | NA | 0.352564931 |
| BC025446 | 0.29422883 | 0.26691138 | 4.26E−85 | 4.45527E−39 | NA | 0.29422883 |
| Nedd4 | 0.377388355 | 0.2599787 | 6.69E−31 | 7.3475E−56 | 0.281232817 | 0.377388355 |
| Lrp1 | 0.505128824 | 0.91404697 | 6.85E−30 | 2.1691E−231 | NA | 0.505128824 |
| Cfi | 0.251606433 | 0.39083407 | 7.2E−30 | 2.1633E−113 | NA | 0.251606433 |
| Cmah | 0.361552447 | 0.25131977 | 5.19E−29 | 6.55013E−28 | NA | 0.361552447 |
| Mlec | 0.324558531 | 0.4003213 | 7.94E−29 | 1.46083E−15 | NA | 0.324558531 |
| Copa | 0.345094339 | 0.29425625 | 1.16E−25 | 1.69118E−64 | NA | 0.345094339 |
| Mtus1 | 0.362060296 | 0.38548314 | 7.17E−25 | 2.92023E−54 | NA | 0.362060296 |
| Mtss1 | 0.287581972 | 0.55213798 | 1.08E−21 | 2.0749E−204 | NA | 0.287581972 |
| Edem1 | 0.286881059 | 0.30121248 | 1.13E−21 | 1.82231E−44 | NA | 0.286881059 |
| Acly | 0.328803101 | 0.28766387 | 1.74E−21 | 4.67378E−64 | NA | 0.328803101 |
| Ptprf | 0.307126385 | 0.27768605 | 6.5E−20 | 4.49155E−33 | NA | 0.307126385 |
| Osbpl9 | 0.278773264 | 0.27071381 | 1.04E−19 | 1.06115E−31 | NA | 0.278773264 |
| Azin1 | 0.270139699 | 0.26889117 | 6.16E−19 | 7.98936E−76 | NA | 0.270139699 |
| Iqgap2 | 0.254698841 | 0.28319294 | 9.72E−19 | 5.91083E−69 | NA | 0.254698841 |
| Prox1 | 0.266329524 | 0.31520499 | 7.66E−14 | 3.0276E−77 | NA | 0.266329524 |
| Caprin1 | 0.262825966 | 0.30302085 | 1.56E−12 | 2.84105E−37 | NA | 0.262825966 |
| Fasn | 0.291406736 | 0.46716808 | 1.44E−09 | 2.5454E−152 | NA | 0.291406736 |
| Apoc2 | 0.782126085 | 0.51714297 | 3.07E−267 | 6.48459E−35 | NA | NA |
| Ahsg | 0.292489576 | 0.32482682 | 1.68E−221 | 3.2651E−222 | NA | NA |
| Hrg | 0.518895355 | 0.58732025 | 3.81E−221 | 3.9739E−209 | NA | NA |
| Agt | 0.520454244 | 0.25820587 | 1.09E−213 | 4.95463E−49 | NA | NA |
| Igfbp4 | 0.43528879 | 0.41117468 | 1.54E−189 | 6.6901E−171 | NA | NA |
| Hspa8 | 0.420379148 | 0.30215243 | 1.8E−143 | 4.30569E−21 | NA | 0.359978165 |
| Serping1 | 0.41280952 | 0.53583075 | 1.56E−134 | 3.5363E−179 | NA | NA |
| Rgs16 | 0.322559157 | 0.43810549 | 5.95E−128 | 3.5457E−188 | NA | NA |
| Vtn | 0.333811366 | 0.51693553 | 2.9E−126 | 8.9644E−294 | NA | NA |
| Gm2a | 0.383109253 | 0.34114593 | 1.13E−105 | 6.60916E−93 | NA | NA |
| Uroc1 | 0.416226092 | 0.45932293 | 3.97E−100 | 1.13457E−82 | NA | NA |
| Snord13 | 0.261078586 | 0.40878863 | 2.42E−99 | 0.000327047 | NA | NA |
| Akr1d1 | 0.361037667 | 0.25829319 | 2.99E−98 | 0.00030931 | 0.278865899 | NA |
| Mgll | 0.333899751 | 0.32184017 | 5.21E−98 | 6.615E−101 | NA | NA |
| Fah | 0.302494707 | 0.3255185 | 1.16E−92 | 1.50517E−71 | 0.259255187 | NA |
| Mttp | 0.311504107 | 0.29665141 | 1.44E−86 | 2.14293E−79 | NA | NA |
| Eci2 | 0.311004843 | 0.28815951 | 1.53E−86 | 5.23022E−88 | NA | NA |
| Igf1 | 0.295791825 | 0.27713876 | 1.83E−78 | 2.85872E−86 | NA | NA |
| C4b | 0.283273876 | 0.82002299 | 3.16E−70 | 0 | NA | NA |
| F9 | 0.256805166 | 0.30398094 | 8.09E−65 | 1.74064E−49 | NA | NA |
| Sardh | 0.269183147 | 0.28626336 | 1.48E−63 | 2.96273E−45 | NA | NA |
| F5 | 0.300566587 | 0.43670918 | 3.21E−60 | 1.06285E−75 | NA | NA |
| Lpin1 | 0.277494868 | 1.32765763 | 1.78E−42 | 0 | NA | NA |
| Glul | 0.379494728 | 1 | 0.00256055 | 1.0077E−186 | NA | NA |

| ID | A48A96logFC | A6p | A24p | A48A96p | PH3logFC | PH48logFC |
|---|---|---|---|---|---|---|
| Mt2 | NA | 1.9E−237 | NA | NA | 2.6060431 | 1.558205591 |
| Mt1 | 0.3563268 | 3.51E−232 | 3.61E−18 | 1.2E−56 | 2.610854994 | 1.733946488 |
| Plin2 | NA | 8.58E−163 | NA | NA | 0.720463076 | NA |
| Il1r1 | NA | 1.47E−162 | NA | NA | 0.998168131 | 0.299449962 |
| Actb | 0.29414 | 6.06E−159 | 5.37E−69 | 8.47E−52 | 0.456981443 | 0.399035393 |
| Cdkn1a | NA | 2.99E−152 | NA | NA | 0.449325471 | NA |
| Klf6 | NA | 1.5E−147 | NA | NA | 0.3616937 | NA |
| Txnrd1 | NA | 1.43E−144 | 1.44E−15 | NA | 0.314979852 | 0.252234489 |
| Gm26924 | NA | 3.7E−144 | 0 | NA | 1.063463323 | 1.445355764 |
| 2010003K11Rik | NA | 2.61E−131 | NA | NA | 0.372219971 | NA |
| Krt18 | NA | 3.97E−121 | NA | NA | NA | 0.520354076 |
| Fga | NA | 2.44E−115 | 0 | NA | 0.981249724 | 0.968748472 |
| Mat1a | 1.0004332 | 3.26E−113 | NA | 0 | 0.623734998 | 0.378728792 |
| n-R5-8s1 | NA | 1.81E−109 | 0 | NA | 1.289598504 | 1.223121237 |
| Lars2 | NA | 9.69E−109 | 1.38E−293 | NA | 0.816795688 | 0.849538102 |
| Myh9 | NA | 3.1E−107 | 2.47E−87 | NA | 0.399128031 | 0.438269269 |
| Fgg | NA | 1.29E−106 | 0 | NA | 0.809322713 | 0.702140119 |
| Gm24601 | NA | 4.45E−106 | 2.17E−275 | NA | 0.924568273 | 0.675379727 |
| Fgb | NA | 4.4E−105 | 0 | NA | 0.739519125 | 0.677592366 |
| Gm15564 | NA | 4.17E−104 | 6.31E−292 | NA | 0.735157137 | 0.746211054 |
| Krt8 | NA | 1.04E−103 | NA | NA | NA | 0.252276759 |
| Clec4f | NA | 2.17E−103 | NA | NA | NA | 0.380337956 |
| Apoa4 | 0.4955793 | 8.56E−101 | NA | 8.65E−87 | 0.609214916 | 1.186863065 |
| Bcl2l1 | NA | 1.02E−99 | NA | NA | 0.280315334 | NA |
| Serpina3m | 0.4325834 | 2.5E−94 | NA | 1.04E−130 | 0.779052745 | 0.519751144 |
| Rcan1 | NA | 1.24E−93 | NA | NA | 0.509239866 | NA |
| D10Wsu102e | NA | 2.09E−90 | NA | NA | 0.256022156 | NA |
| Slc39a14 | NA | 2.25E−81 | 8.94E−22 | NA | 0.831584936 | NA |
| C1qb | NA | 1.02E−79 | NA | NA | NA | 0.361309767 |
| Tmsb4x | NA | 7.99E−79 | NA | NA | NA | 0.347657914 |
| Ctss | NA | 2.26E−78 | NA | NA | NA | NA |
| Mrap | NA | 2.65E−73 | NA | NA | NA | 0.280974611 |
| Hnf4a | 0.3147106 | 6.36E−71 | 9.91E−71 | 3.56E−83 | 0.644195253 | NA |
| Csf1r | NA | 4.7E−70 | NA | NA | NA | 0.284684372 |
| Trp53inp1 | NA | 1.52E−67 | 2.95E−29 | NA | 0.269278401 | NA |
| Nop58 | NA | 2.13E−65 | NA | NA | 0.257566653 | NA |
| Mfsd2a | 0.6122327 | 7.88E−65 | NA | 5.34E−233 | 0.496351884 | NA |
| Lyz2 | NA | 2.49E−63 | NA | NA | NA | 0.275390862 |
| Bach1 | NA | 2.51E−63 | NA | NA | 0.533237374 | NA |
| Fam134b | NA | 5.02E−63 | NA | NA | 0.865578177 | NA |
| Cd5l | NA | 1.08E−62 | NA | NA | NA | 0.460825248 |
| Serpina3n | 0.3464828 | 4.98E−61 | 4.35E−45 | 9.93E−85 | 1.759758939 | 1.082720183 |
| Apob | NA | 5.69E−61 | 4.36E−68 | NA | 0.416824707 | NA |
| Eif1a | NA | 1.8E−60 | NA | NA | 0.295219945 | NA |
| Epas1 | NA | 7.57E−60 | NA | NA | 0.38425941 | NA |
| Sdc4 | NA | 9.47E−59 | NA | NA | 0.43560504 | NA |
| C3 | 0.2591667 | 1.55E−58 | 1.23E−125 | 1.44E−100 | 0.482674202 | 0.982337034 |
| Wfdc17 | NA | 2.57E−58 | NA | NA | NA | NA |
| Fkbp5 | NA | 1.44E−57 | NA | NA | 0.663304836 | NA |
| Btg2 | NA | 3.81E−56 | NA | NA | 0.772686832 | 0.280923811 |
| Tiparp | NA | 8.73E−55 | NA | NA | 0.489122153 | NA |
| Cldn14 | NA | 1.86E−52 | NA | NA | 0.511583526 | NA |
| Abhd2 | NA | 1.13E−50 | NA | NA | NA | NA |
| Picalm | NA | 1.39E−50 | NA | NA | 0.276902165 | NA |
| Igfbp1 | NA | 1.68E−50 | 9.05E−32 | NA | 1.288789902 | 0.466237818 |
| Stat3 | NA | 2.74E−50 | 8.01E−18 | NA | 0.460515229 | 0.308012615 |
| Myo1e | NA | 9.34E−50 | NA | NA | 0.399456672 | NA |
| Tmem87b | NA | 1.07E−49 | NA | NA | 0.288330587 | NA |
| Slc10a2 | NA | 2.04E−49 | NA | NA | 0.30563824 | NA |
| Lcn2 | NA | 2.72E−49 | NA | NA | 1.499141183 | 2.39758181 |
| Jmjd1c | NA | 3.43E−49 | NA | NA | 0.39612601 | NA |
| Itih3 | NA | 1.4E−47 | 1.11E−89 | NA | 0.469631651 | 1.223022315 |
| Tacc2 | NA | 3.95E−42 | NA | NA | 0.877623973 | NA |
| Eef2 | NA | 2.71E−41 | 2.54E−28 | NA | 0.289562847 | NA |
| Ptpn1 | NA | 3.51E−41 | NA | NA | 0.268263225 | NA |
| Tnfrsf1a | NA | 3.86E−41 | NA | NA | 0.346845029 | NA |
| Ddx3x | NA | 1.07E−40 | NA | NA | 0.47100162 | NA |
| Itih2 | NA | 3.43E−38 | 6.17E−35 | NA | 0.336714211 | 0.312933325 |
| St3gal5 | 0.5423784 | 6.13E−38 | NA | 1.06E−170 | 0.882761517 | NA |
| Abca1 | NA | 8.81E−38 | 4.14E−37 | NA | 0.50506234 | NA |
| Bcl3 | NA | 3.63E−37 | NA | NA | 0.419650143 | NA |
| Asl | 0.4384583 | 8.13E−37 | NA | 3.45E−117 | 0.297673772 | NA |
| Aldob | 0.3654606 | 1.58E−35 | NA | 9.03E−178 | 0.490200848 | 0.254127817 |
| Ugdh | NA | 5.17E−34 | NA | NA | NA | 0.260443532 |
| H3f3b | NA | 7.03E−34 | NA | NA | 0.755567107 | NA |
| Xbp1 | NA | 9.59E−34 | NA | NA | 0.539986 | NA |
| Ctsl | NA | 1.93E−33 | NA | NA | 0.256520866 | NA |
| Hspb8 | NA | 2E−33 | NA | NA | 0.667847253 | NA |
| Eif4g2 | NA | 5.94E−33 | 1.62E−67 | NA | 0.384302798 | NA |
| Epb4.1 | NA | 1.67E−30 | NA | NA | 0.349430673 | NA |
| Rnd3 | NA | 3.93E−30 | NA | NA | 0.285147682 | NA |
| Ddx5 | NA | 5.9E−30 | NA | NA | 0.251675045 | NA |
| Plec | NA | 8.3E−30 | NA | NA | NA | NA |
| Jak1 | NA | 1.34E−29 | 1.2E−17 | NA | 0.362766687 | NA |
| Errfi1 | NA | 1.52E−29 | NA | NA | 0.500850526 | NA |
| Iigp1 | NA | 2.65E−29 | 7.59E−66 | NA | NA | NA |
| Lrg1 | NA | 1.92E−28 | 1.37E−62 | NA | 0.870250525 | 1.051046086 |
| Gpt2 | NA | 2.49E−28 | NA | NA | 0.533331785 | NA |
| Gjb2 | NA | 1.12E−27 | 3.74E−38 | NA | 0.639507436 | 0.328679811 |
| Dst | NA | 2.08E−26 | 1.75E−35 | NA | 0.329905553 | NA |
| Wbp1l | 0.4404629 | 2.48E−26 | NA | 4.08E−134 | 0.409138156 | NA |
| Fn1 | 0.2925222 | 2.99E−26 | 1.87E−106 | 3.08E−64 | 0.684266092 | 0.469732274 |
| Grn | 0.4732523 | 1.38E−25 | NA | 1.07E−154 | 0.403315761 | 0.293077345 |
| Mpeg1 | NA | 1.8E−24 | NA | NA | NA | NA |
| Aff4 | NA | 1.96E−24 | NA | NA | 0.344186222 | NA |
| Gm26917 | NA | 6.5E−24 | 5.26E−54 | NA | 0.46739621 | 0.475554164 |
| Tns1 | NA | 8.9E−24 | NA | NA | 0.33981795 | NA |
| Arg1 | NA | 8.62E−23 | NA | NA | 0.381616351 | NA |
| Arhgef12 | NA | 8.83E−23 | 1.41E−20 | NA | 0.280147378 | NA |
| Man2a1 | 0.3327558 | 6.13E−22 | 2.79E−19 | 2.32E−81 | 0.369999358 | NA |
| Lpgat1 | 0.2576004 | 4.45E−21 | 3.86E−28 | 3.53E−63 | 0.390146943 | 0.347767347 |
| Il6ra | NA | 5.08E−20 | NA | NA | 0.251696836 | NA |
| Ptp4a1 | NA | 3.82E−19 | NA | NA | 0.393137367 | NA |
| Atp2a2 | 0.3056362 | 2.9E−18 | NA | 7.58E−69 | 0.350363266 | NA |
| Aass | NA | 3.07E−18 | NA | NA | 0.705615345 | NA |
| Nedd4l | NA | 4.9E−18 | NA | NA | 0.271827098 | NA |
| Egr1 | NA | 2.8E−17 | NA | NA | 0.620480469 | NA |
| Litaf | NA | 2.04E−16 | NA | NA | 0.396007707 | NA |
| Plk3 | NA | 3.12E−16 | NA | NA | 0.488016367 | NA |
| Slc41a2 | NA | 6.42E−16 | 2.13E−34 | NA | 0.3685439 | 0.318516467 |
| Hsp90ab1 | NA | 8.34E−16 | NA | NA | NA | 0.455851506 |
| Tgm2 | NA | 1.43E−15 | NA | NA | 0.316575272 | 0.37123494 |
| Shb | NA | 4.68E−15 | NA | NA | 0.430613558 | NA |
| Tmed5 | 0.3450078 | 1.17E−13 | NA | 5.01E−102 | 0.296018777 | NA |
| Atp1b1 | NA | 3.27E−13 | NA | NA | 0.424801231 | NA |
| Ctsb | NA | 4.83E−13 | NA | NA | 0.268200419 | 0.310468937 |
| Mug2 | NA | 9.47E−13 | 6.96E−26 | NA | NA | NA |
| Slc25a47 | 0.5026615 | 2.18E−12 | NA | 1.45E−158 | 0.635928777 | 0.555532536 |
| Atf6 | NA | 7.66E−12 | NA | NA | 0.280521162 | NA |
| Ell2 | NA | 8.49E−12 | 2.55E−22 | NA | 0.371745977 | NA |
| Brd2 | NA | 1.73E−11 | 2.65E−12 | NA | 0.258446853 | NA |
| Snx10 | NA | 2.35E−11 | NA | NA | 0.558244633 | 0.253822304 |
| Steap4 | NA | 3.54E−11 | 6.59E−69 | NA | 1.890015167 | 0.631013696 |
| Gfra1 | 0.3208433 | 8.44E−11 | NA | 2.65E−92 | 0.474694426 | NA |
| Cp | 0.4362433 | 2.41E−10 | 1.74E−45 | 6.52E−192 | NA | 0.620221202 |
| Slc25a51 | NA | 6.14E−10 | NA | NA | 0.372157664 | NA |
| Eef1a1 | NA | 1.02E−09 | 2.2E−81 | NA | 0.340768186 | 0.254969818 |
| Ivns1abp | NA | 1.2E−09 | NA | NA | 0.46254094 | NA |
| Por | 0.3319492 | 4.49E−09 | 2.42E−08 | 1.06E−73 | 0.290745567 | NA |
| Plin5 | 0.3520871 | 6.88E−09 | 2.38E−16 | 1.22E−125 | 0.489257529 | NA |
| Ppip5k2 | NA | 7.41E−09 | NA | NA | 0.30299148 | NA |
| Fmo5 | 0.2520709 | 1.05E−08 | NA | 4.71E−64 | 0.690160801 | 0.379271143 |
| Ccnl1 | NA | 1.44E−08 | NA | NA | 0.341338448 | NA |
| Nrp1 | NA | 2.25E−08 | 1.93E−28 | NA | 0.268796793 | NA |
| H2-D1 | NA | 2.31E−08 | NA | NA | NA | NA |
| Cxcl1 | 0.2572501 | 4.89E−08 | 1.26E−30 | 9.38E−68 | 1.230398019 | 0.353012683 |
| Hdlbp | NA | 0.000000054 | 5.1E−95 | NA | 0.267317937 | NA |
| Hspa5 | 0.2911841 | 9.35E−08 | 9.07E−184 | 2.27E−84 | NA | 0.270882455 |
| Atp1a1 | 0.2627704 | 0.000000242 | 0.00000197 | 3.57E−63 | 0.261718912 | NA |
| Nucks1 | NA | 0.000000282 | NA | NA | NA | 0.271055696 |
| Ctnnd1 | NA | 0.000000548 | NA | NA | NA | NA |
| Sult1a1 | 0.5604535 | 0.000000664 | NA | 9.07E−216 | 0.371023537 | 0.344836675 |
| H6pd | NA | 0.000000926 | 2.22E−16 | NA | 0.300492669 | NA |
| Serinc3 | 0.2923207 | 0.00000117 | NA | 4.67E−75 | 0.293964974 | NA |
| Il6st | NA | 0.00000169 | 3.02E−29 | NA | 0.262348153 | NA |
| Elovl5 | NA | 0.00000254 | NA | NA | 0.382267188 | NA |
| Cyp3a13 | NA | 0.00000493 | NA | NA | NA | NA |
| Rmnd5a | NA | 0.0000109 | NA | NA | 0.273473023 | NA |
| mmu-mir-6236 | 0.429737 | 0.0000218 | 7.24E−35 | 2E−10 | 0.468866739 | 0.632369146 |
| Saa2 | 0.390082 | 0.000262015 | 8.96E−95 | 6.06E−64 | 2.971862499 | 4.43818759 |
| Cpt1a | 0.3729352 | 0.000289401 | NA | 2.13E−98 | 0.42860734 | NA |
| Acsl4 | NA | 0.000351815 | 1.14E−25 | NA | 0.250793464 | 0.273275871 |
| Trib1 | NA | 0.001450331 | 7.49E−21 | NA | 0.581754281 | NA |
| Psap | NA | 0.360973931 | NA | NA | NA | NA |
| Apcs | NA | 0.75840106 | 2.16E−78 | NA | 0.573439593 | 2.049485412 |
| Gm23935 | 0.5019442 | 1 | 0.068577969 | 3.7E−142 | 0.498066925 | 0.562414557 |
| Sephs2 | NA | NA | 2.83E−263 | NA | 0.618707003 | NA |
| Fgl1 | NA | NA | 1.6E−238 | NA | 0.49338252 | 1.561880768 |
| Hyou1 | NA | NA | 1.62E−156 | NA | 0.431109873 | 0.315760783 |
| Calr | NA | NA | 1.67E−148 | NA | NA | NA |
| Scd1 | 0.5192181 | 5.04E−19 | 2.08E−139 | 5.06E−126 | 1.081676999 | NA |
| Itih4 | NA | NA | 4.26E−137 | NA | 0.887186241 | 1.171742648 |
| Hp | NA | NA | 8.95E−137 | NA | 0.774785279 | 1.584889895 |
| Pdia3 | NA | NA | 7.36E−136 | NA | NA | NA |
| P4hb | 0.2506609 | NA | 2.27E−113 | 1.25E−84 | NA | 0.38506968 |
| Glud1 | NA | NA | 6.55E−110 | NA | 0.341724783 | 0.359099708 |
| Pzp | NA | NA | 3.34E−97 | NA | 0.387351113 | 0.382796555 |
| Serpina10 | NA | NA | 8.21E−94 | NA | 0.733655099 | 0.871044179 |
| Creld2 | NA | NA | 1.18E−91 | NA | NA | 0.362477611 |
| Egfr | 0.473389 | NA | 1.72E−79 | 5.82E−71 | 0.555834247 | NA |
| Orm1 | 0.310039 | NA | 4.87E−78 | 1.56E−69 | 0.463411533 | 1.823760769 |
| Kng1 | NA | NA | 1.32E−71 | NA | NA | 0.419944891 |
| Elovl2 | NA | NA | 1.22E−62 | NA | 0.285038606 | NA |
| Cps1 | NA | NA | 1.45E−55 | NA | 0.336429448 | NA |
| Prg4 | NA | NA | 3.2E−54 | NA | 0.480504574 | 0.355545985 |
| Manf | NA | NA | 1.6E−53 | NA | NA | 0.282950631 |
| Orm2 | NA | NA | 2.71E−53 | NA | NA | 2.405086745 |
| Cfh | 0.306326 | NA | 3.24E−52 | 2.09E−34 | 0.312615767 | 0.588715272 |
| Hc | NA | NA | 1.11E−51 | NA | NA | NA |
| Dhcr24 | 0.3872743 | NA | 3.44E−48 | 3.03E−107 | NA | 0.759730578 |
| Cpb2 | NA | 4.76E−14 | 5.47E−45 | NA | 0.280302458 | 0.395699062 |
| Plg | NA | NA | 1.34E−44 | NA | 0.26619661 | NA |
| Acox1 | NA | NA | 1.78E−40 | NA | 0.540022158 | NA |
| Dnajc12 | NA | NA | 1.14E−39 | NA | NA | 0.312907586 |
| C8a | 0.327131 | NA | 3.84E−39 | 7.61E−55 | 0.294064237 | NA |
| Qsox1 | NA | NA | 9.2E−39 | NA | 0.378486857 | 0.551776463 |
| Slc3a1 | NA | NA | 7.1E−38 | NA | 0.290117272 | 0.321802388 |
| Fos | NA | NA | 1.43E−36 | NA | 0.524733327 | NA |
| Dstn | NA | NA | 1.75E−35 | NA | 0.271381717 | NA |
| Cyb5r3 | NA | NA | 3.1E−34 | NA | 0.337684684 | NA |
| Efna1 | NA | NA | 1.73E−31 | NA | 0.256152989 | NA |
| Slc7a2 | 0.3449781 | NA | 1.75E−31 | 3.47E−68 | 0.622326529 | NA |
| BC025446 | 0.2726109 | NA | 6.33E−31 | 4.26E−85 | NA | NA |
| Nedd4 | NA | 4.9E−18 | 6.69E−31 | NA | 0.259978699 | NA |
| Lrp1 | NA | NA | 6.85E−30 | NA | 0.310226628 | 0.314138344 |
| Cfi | NA | NA | 7.2E−30 | NA | NA | NA |
| Cmah | NA | NA | 5.19E−29 | NA | NA | NA |
| Mlec | NA | NA | 7.94E−29 | NA | NA | 0.400321297 |
| Copa | NA | NA | 1.16E−25 | NA | 0.251410027 | NA |
| Mtus1 | NA | NA | 7.17E−25 | NA | 0.268076242 | NA |
| Mtss1 | NA | NA | 1.08E−21 | NA | 0.552137981 | NA |
| Edem1 | NA | NA | 1.13E−21 | NA | NA | NA |
| Acly | NA | NA | 1.74E−21 | NA | 0.287663867 | NA |
| Ptprf | NA | NA | 6.5E−20 | NA | NA | NA |
| Osbpl9 | NA | NA | 1.04E−19 | NA | NA | NA |
| Azin1 | NA | NA | 6.16E−19 | NA | 0.268891169 | NA |
| Iqgap2 | NA | NA | 9.72E−19 | NA | 0.283192936 | NA |
| Prox1 | NA | NA | 7.66E−14 | NA | 0.31520499 | NA |
| Caprin1 | NA | NA | 1.56E−12 | NA | NA | 0.303020851 |
| Fasn | NA | NA | 1.44E−09 | NA | 0.467168076 | NA |
| Apoc2 | 0.7821261 | NA | NA | 3.07E−267 | NA | 0.517142972 |
| Ahsg | 0.2924896 | NA | NA | 1.68E−221 | NA | 0.281450293 |
| Hrg | 0.5188954 | NA | NA | 3.81E−221 | NA | 0.270451116 |
| Agt | 0.5204542 | NA | NA | 1.09E−213 | NA | NA |
| Igfbp4 | 0.4352888 | NA | NA | 1.54E−189 | 0.387861679 | NA |
| Hspa8 | 0.4203791 | NA | 1.12E−41 | 1.8E−143 | NA | 0.302152434 |
| Serping1 | 0.4128095 | NA | NA | 1.56E−134 | NA | NA |
| Rgs16 | 0.3225592 | NA | NA | 5.95E−128 | 0.438105488 | NA |
| Vtn | 0.3338114 | NA | NA | 2.9E−126 | NA | NA |
| Gm2a | 0.3831093 | NA | NA | 1.13E−105 | 0.341145926 | NA |
| Uroc1 | 0.4162261 | NA | NA | 3.97E−100 | 0.329587334 | NA |
| Snord13 | 0.2610786 | NA | NA | 2.42E−99 | NA | 0.408788625 |
| Akr1d1 | 0.3610377 | 6.11E−10 | NA | 2.99E−98 | NA | 0.258293189 |
| Mgll | 0.3338998 | NA | NA | 5.21E−98 | 0.321840174 | NA |
| Fah | 0.3024947 | 1.82E−10 | NA | 1.16E−92 | NA | NA |
| Mttp | 0.3115041 | NA | NA | 1.44E−86 | 0.296651415 | NA |
| Eci2 | 0.3110048 | NA | NA | 1.53E−86 | 0.288159513 | NA |
| Igf1 | 0.2957918 | NA | NA | 1.83E−78 | 0.277138761 | NA |
| C4b | 0.2832739 | NA | NA | 3.16E−70 | 0.26668764 | 0.530516995 |
| F9 | 0.2568052 | NA | NA | 8.09E−65 | NA | NA |
| Sardh | 0.2691831 | NA | NA | 1.48E−63 | NA | NA |
| F5 | 0.3005666 | NA | NA | 3.21E−60 | NA | NA |
| Lpin1 | 0.2774949 | NA | NA | 1.78E−42 | 1.327657625 | NA |
| Glul | 0.3794947 | NA | NA | 0.00256055 | 0.478908893 | 0.611936929 |

| ID | PH120logFC | PH3p | PH48p | PH120p |
|---|---|---|---|---|
| Mt2 | 1.471840722 | 0 | 2.0362E−271 | 0 |
| Mt1 | 1.65972779 | 0 | 9.8777E−293 | 0 |
| Plin2 | NA | 0 | NA | NA |
| Il1r1 | 0.63980318 | 0 | 2.79338E−18 | 4.6854E−177 |
| Actb | NA | 2.1478E−210 | 2.62127E−44 | NA |
| Cdkn1a | NA | 7.064E−169 | NA | NA |
| Klf6 | NA | 8.0005E−203 | NA | NA |
| Txnrd1 | NA | 3.22341E−84 | 0.032529357 | NA |
| Gm26924 | 1.379756734 | 0 | 0 | 0 |
| 2010003K11Rik | NA | 1.0574E−239 | NA | NA |
| Krt18 | NA | NA | 5.88314E−34 | NA |
| Fga | 1.039432587 | 0 | 9.2714E−246 | 0 |
| Mat1a | 0.519634823 | 0 | 5.65122E−41 | 2.896E−246 |
| n-R5-8s1 | 1.066654791 | 0 | 2.9367E−283 | 0 |
| Lars2 | 1.419129056 | 0 | 4.7257E−222 | 0 |
| Myh9 | 0.631035657 | 3.0354E−126 | 1.59002E−15 | 2.8315E−141 |
| Fgg | 0.686743278 | 0 | 3.4215E−167 | 0 |
| Gm24601 | 0.675086715 | 0 | 2.6798E−176 | 0 |
| Fgb | 0.605783409 | 0 | 1.4129E−179 | 0 |
| Gm15564 | 1.485885928 | 0 | 4.3231E−146 | 0 |
| Krt8 | NA | NA | 0.182829207 | NA |
| Clec4f | 0.487336922 | NA | 1.17731E−53 | 1.7326E−119 |
| Apoa4 | NA | 2.34147E−92 | 3.0126E−137 | NA |
| Bcl2l1 | NA | 1.1685E−128 | NA | NA |
| Serpina3m | 0.305171041 | 0 | 1.275E−57 | 2.79622E−48 |
| Rcan1 | NA | 4.5798E−279 | NA | NA |
| D10Wsu102e | NA | 1.0336E−141 | NA | NA |
| Slc39a14 | 0.506662289 | 0 | NA | 1.1603E−108 |
| C1qb | 0.337494489 | NA | 2.5047E−59 | 6.3479E−100 |
| Tmsb4x | 0.341035762 | NA | 7.53442E−31 | 1.30373E−70 |
| Ctss | 0.270183132 | NA | NA | 2.67839E−57 |
| Mrap | NA | NA | 1.03675E−10 | NA |
| Hnf4a | 0.470873585 | 5.6829E−290 | NA | 3.0788E−110 |
| Csf1r | NA | NA | 3.78986E−37 | NA |
| Trp53inp1 | NA | 1.01347E−72 | NA | NA |
| Nop58 | NA | 1.11973E−93 | NA | NA |
| Mfsd2a | NA | 1.6404E−219 | NA | NA |
| Lyz2 | 0.402735723 | NA | 1.20955E−28 | 3.32908E−93 |
| Bach1 | NA | 5.5373E−247 | NA | NA |
| Fam134b | NA | 0 | NA | NA |
| Cd5l | 0.49371428 | NA | 2.074E−79 | 4.6843E−138 |
| Serpina3n | 1.205364333 | 0 | 4.6706E−169 | 0 |
| Apob | 0.505397578 | 8.048E−169 | NA | 4.6275E−151 |
| Eif1a | NA | 7.80345E−96 | NA | NA |
| Epas1 | NA | 2.1215E−138 | NA | NA |
| Sdc4 | 0.315436107 | 2.7024E−193 | NA | 2.09944E−74 |
| C3 | 1.071950932 | 0 | 0 | 0 |
| Wfdc17 | 0.317329894 | NA | NA | 1.3239E−101 |
| Fkbp5 | NA | 0 | NA | NA |
| Btg2 | 0.386164963 | 7.2437E−290 | 2.37272E−05 | 2.69258E−68 |
| Tiparp | NA | 7.0657E−236 | NA | NA |
| Cldn14 | NA | 4.316E−281 | NA | NA |
| Abhd2 | 0.267241512 | NA | NA | 1.29723E−66 |
| Picalm | NA | 1.27269E−76 | NA | NA |
| Igfbp1 | 0.488899754 | 0 | 2.67298E−25 | 1.15024E−78 |
| Stat3 | 0.483904634 | 3.5753E−174 | 2.82354E−05 | 1.7545E−98 |
| Myo1e | NA | 7.8258E−162 | NA | NA |
| Tmem87b | NA | 8.7183E−155 | NA | NA |
| Slc10a2 | NA | 6.538E−197 | NA | NA |
| Lcn2 | 2.127649068 | 0 | 0 | 0 |
| Jmjd1c | NA | 1.0512E−144 | NA | NA |
| Itih3 | 1.177912468 | 1.1211E−197 | 4.0411E−297 | 0 |
| Tacc2 | NA | 0 | NA | NA |
| Eef2 | 0.321705764 | 1.70104E−97 | NA | 1.01772E−72 |
| Ptpn1 | NA | 6.9212E−130 | NA | NA |
| Tnfrsf1a | 0.267537895 | 3.4204E−112 | NA | 1.90007E−43 |
| Ddx3x | NA | 1.3581E−155 | NA | NA |
| Itih2 | NA | 1.5587E−122 | 2.29359E−24 | NA |
| St3gal5 | NA | 0 | NA | NA |
| Abca1 | 0.370856836 | 3.1915E−172 | NA | 2.16402E−52 |
| Bcl3 | NA | 3.0494E−251 | NA | NA |
| Asl | NA | 1.76334E−83 | NA | NA |
| Aldob | NA | 0 | 2.24769E−64 | NA |
| Ugdh | NA | NA | 4.68355E−05 | NA |
| H3f3b | NA | 4.4441E−288 | NA | NA |
| Xbp1 | NA | 1.3385E−208 | NA | NA |
| Ctsl | NA | 1.56147E−59 | NA | NA |
| Hspb8 | 0.52383406 | 9.1859E−267 | NA | 4.2261E−133 |
| Eif4g2 | 0.287105046 | 2.505E−110 | NA | 8.06734E−39 |
| Epb4.1 | NA | 1.9295E−113 | NA | NA |
| Rnd3 | NA | 4.6581E−134 | NA | NA |
| Ddx5 | NA | 3.02351E−75 | NA | NA |
| Plec | 0.267122123 | NA | NA | 2.03726E−37 |
| Jak1 | NA | 2.4382E−103 | NA | NA |
| Errfi1 | NA | 7.4336E−262 | NA | NA |
| Iigp1 | 0.305889089 | NA | NA | 4.94888E−49 |
| Lrg1 | 1.260509735 | 0 | 1.5393E−213 | 0 |
| Gpt2 | NA | 3.6697E−218 | NA | NA |
| Gjb2 | 0.402811227 | 9.9227E−255 | 2.44199E−12 | 1.87917E−70 |
| Dst | NA | 7.3975E−105 | NA | NA |
| Wbp1l | NA | 1.2925E−127 | NA | NA |
| Fn1 | 1.018772906 | 5.5621E−298 | 5.12048E−32 | 0 |
| Grn | 0.316049742 | 3.4391E−128 | 4.46789E−10 | 1.12476E−47 |
| Mpeg1 | 0.541170007 | NA | NA | 4.2393E−174 |
| Aff4 | NA | 7.00106E−97 | NA | NA |
| Gm26917 | 0.443747552 | 2.4164E−144 | 2.51697E−05 | 7.15171E−51 |
| Tns1 | NA | 4.5763E−128 | NA | NA |
| Arg1 | NA | 2.227E−218 | NA | NA |
| Arhgef12 | 0.304865834 | 1.49183E−67 | NA | 1.25661E−32 |
| Man2a1 | 0.323273082 | 3.60298E−98 | NA | 1.18903E−46 |
| Lpgat1 | 0.400787984 | 1.2472E−107 | 1.05076E−09 | 1.04662E−64 |
| Il6ra | NA | 2.9323E−108 | NA | NA |
| Ptp4a1 | NA | 6.0997E−190 | NA | NA |
| Atp2a2 | 0.263222967 | 8.21385E−94 | NA | 4.4193E−29 |
| Aass | 0.312355615 | 1.0663E−286 | NA | 3.70382E−47 |
| Nedd4l | NA | 1.5124E−116 | NA | NA |
| Egr1 | 0.271667356 | 7.2676E−185 | NA | 3.36174E−20 |
| Litaf | NA | 2.337E−132 | NA | NA |
| Plk3 | NA | 8.2222E−205 | NA | NA |
| Slc41a2 | 0.441321771 | 2.2246E−166 | 5.59542E−25 | 4.3631E−127 |
| Hsp90ab1 | 0.323037124 | NA | 1.23192E−83 | 1.16529E−94 |
| Tgm2 | 0.342526865 | 5.49556E−85 | 5.93696E−16 | 2.52488E−59 |
| Shb | NA | 4.0274E−182 | NA | NA |
| Tmed5 | NA | 1.362E−80 | NA | NA |
| Atp1b1 | NA | 1.3155E−145 | NA | NA |
| Ctsb | 0.553974491 | 1.08822E−72 | 2.1673E−12 | 4.5123E−149 |
| Mug2 | 0.536577024 | NA | NA | 3.3989E−113 |
| Slc25a47 | NA | 0 | 2.8075E−104 | NA |
| Atf6 | NA | 1.54665E−82 | NA | NA |
| Ell2 | NA | 9.305E−113 | NA | NA |
| Brd2 | NA | 8.83728E−51 | NA | NA |
| Snx10 | NA | 1.5948E−251 | 1.27886E−13 | NA |
| Steap4 | 0.931387724 | 0 | 4.71389E−63 | 1.3111E−284 |
| Gfra1 | NA | 4.734E−168 | NA | NA |
| Cp | 0.655511152 | NA | 1.411E−115 | 5.8053E−290 |
| Slc25a51 | NA | 1.1132E−164 | NA | NA |
| Eef1a1 | NA | 6.1414E−119 | 1.24947E−12 | NA |
| Ivns1abp | NA | 9.4406E−168 | NA | NA |
| Por | NA | 3.92838E−83 | NA | NA |
| Plin5 | NA | 1.0072E−205 | NA | NA |
| Ppip5k2 | NA | 3.6258E−115 | NA | NA |
| Fmo5 | 0.337493236 | 0 | 1.75071E−43 | 5.95154E−73 |
| Ccnl1 | NA | 1.6662E−120 | NA | NA |
| Nrp1 | NA | 7.49426E−79 | NA | NA |
| H2-D1 | 0.433183706 | NA | NA | 1.1359E−105 |
| Cxcl1 | NA | 0 | 7.28481E−05 | NA |
| Hdlbp | 0.394030651 | 3.33056E−59 | NA | 2.52472E−71 |
| Hspa5 | 0.339769129 | NA | 2.26522E−12 | 1.02395E−98 |
| Atp1a1 | NA | 2.95645E−65 | NA | NA |
| Nucks1 | NA | NA | 1 | NA |
| Ctnnd1 | 0.252846161 | NA | NA | 1.41477E−35 |
| Sult1a1 | NA | 2.6725E−99 | 2.16219E−12 | NA |
| H6pd | 0.491965789 | 1.9138E−81 | NA | 1.0068E−111 |
| Serinc3 | NA | 2.70765E−85 | NA | NA |
| Il6st | 0.382881893 | 3.02061E−73 | NA | 3.34974E−58 |
| Elovl5 | NA | 1.0806E−102 | NA | NA |
| Cyp3a13 | 0.420680082 | NA | NA | 6.40777E−81 |
| Rmnd5a | NA | 5.48218E−73 | NA | NA |
| mmu-mir-6236 | 1.257248389 | 4.5061E−221 | 5.48505E−97 | 5.137E−284 |
| Saa2 | 3.598008691 | 0 | 0 | 0 |
| Cpt1a | 0.276570679 | 3.3126E−139 | NA | 1.18854E−34 |
| Acsl4 | NA | 5.43114E−70 | 0.003086449 | NA |
| Trib1 | NA | 1.5671E−204 | NA | NA |
| Psap | 0.318472244 | NA | NA | 5.29626E−54 |
| Apcs | 0.965147497 | 2.0613E−182 | 0 | 5.9431E−275 |
| Gm23935 | 1.207244314 | 8.5684E−162 | 2.20888E−73 | 1.3842E−297 |
| Sephs2 | 0.604852301 | 1.0636E−281 | NA | 1.8569E−182 |
| Fgl1 | 1.379926505 | 2.3577E−175 | 0 | 0 |
| Hyou1 | 0.496156079 | 3.5998E−141 | 0.000165574 | 3.38682E−91 |
| Calr | 0.391955529 | NA | NA | 1.6119E−116 |
| Scd1 | 0.787835318 | 0 | NA | 3.7098E−204 |
| Itih4 | 1.18011817 | 0 | 0 | 0 |
| Hp | 1.107075607 | 0 | 0 | 0 |
| Pdia3 | 0.276156858 | NA | NA | 3.3607E−53 |
| P4hb | 0.369961971 | NA | 1.73293E−74 | 1.31E−132 |
| Glud1 | 0.307823999 | 1.1097E−139 | 4.69364E−47 | 1.29722E−69 |
| Pzp | 0.636851805 | 2.8474E−237 | 2.61472E−52 | 0 |
| Serpina10 | 0.740578005 | 0 | 3.5999E−107 | 1.3451E−230 |
| Creld2 | 0.307177214 | NA | 1.11415E−08 | 5.90495E−44 |
| Egfr | 0.65440823 | 5.4692E−228 | NA | 4.2603E−199 |
| Orm1 | 1.05827481 | 4.577E−180 | 0 | 0 |
| Kng1 | 0.546835494 | NA | 3.9248E−102 | 0 |
| Elovl2 | NA | 2.60256E−69 | NA | NA |
| Cps1 | 0.312297912 | 1.1876E−145 | NA | 8.38552E−77 |
| Prg4 | 0.810835665 | 4.3499E−189 | 1.45301E−24 | 8.3602E−244 |
| Manf | NA | NA | 0.023268676 | NA |
| Orm2 | 1.536747401 | NA | 0 | 0 |
| Cfh | 0.83288399 | 3.4561E−113 | 2.5189E−103 | 0 |
| Hc | 0.611232959 | NA | NA | 2.3088E−207 |
| Dhcr24 | 0.551421264 | NA | 1.6476E−115 | 1.4499E−130 |
| Cpb2 | 0.403790403 | 1.58681E−74 | 1.08559E−38 | 3.3355E−100 |
| Plg | 0.596524395 | 1.3469E−121 | NA | 0 |
| Acox1 | 0.292890336 | 0 | NA | 1.80286E−68 |
| Dnajc12 | NA | NA | 2.5383E−30 | NA |
| C8a | 0.404198047 | 1.39461E−84 | NA | 2.99834E−96 |
| Qsox1 | 0.725062115 | 3.7529E−121 | 2.238E−55 | 2.2371E−241 |
| Slc3a1 | 0.381791335 | 1.93288E−94 | 1.78412E−11 | 2.58695E−55 |
| Fos | NA | 3.547E−125 | NA | NA |
| Dstn | NA | 2.89799E−82 | NA | NA |
| Cyb5r3 | NA | 1.89897E−92 | NA | NA |
| Efna1 | 0.268455179 | 2.54995E−60 | NA | 2.85495E−25 |
| Slc7a2 | 0.374380964 | 6.7383E−265 | NA | 2.68204E−62 |
| BC025446 | 0.266911384 | NA | NA | 4.45527E−39 |
| Nedd4 | NA | 7.3475E−56 | NA | NA |
| Lrp1 | 0.914046975 | 2.25364E−68 | 2.74872E−05 | 2.1691E−231 |
| Cfi | 0.390834066 | NA | NA | 2.1633E−113 |
| Cmah | 0.251319774 | NA | NA | 6.55013E−28 |
| Mlec | NA | NA | 1.46083E−15 | NA |
| Copa | 0.294256247 | 1.69118E−64 | NA | 4.74634E−40 |
| Mtus1 | 0.385483144 | 2.92023E−54 | NA | 4.22517E−49 |
| Mtss1 | 0.339863924 | 2.0749E−204 | NA | 1.12341E−54 |
| Edem1 | 0.301212478 | NA | NA | 1.82231E−44 |
| Acly | NA | 4.67378E−64 | NA | NA |
| Ptprf | 0.27768605 | NA | NA | 4.49155E−33 |
| Osbpl9 | 0.270713808 | NA | NA | 1.06115E−31 |
| Azin1 | NA | 7.98936E−76 | NA | NA |
| Iqgap2 | NA | 5.91083E−69 | NA | NA |
| Prox1 | NA | 3.0276E−77 | NA | NA |
| Caprin1 | 0.295892842 | NA | 0.016747307 | 2.84105E−37 |
| Fasn | NA | 2.5454E−152 | NA | NA |
| Apoc2 | NA | NA | 6.48459E−35 | NA |
| Ahsg | 0.32482682 | NA | 7.28765E−69 | 3.2651E−222 |
| Hrg | 0.58732025 | NA | 6.86112E−14 | 3.9739E−209 |
| Agt | 0.258205872 | NA | NA | 4.95463E−49 |
| Igfbp4 | 0.411174683 | 6.6901E−171 | NA | 1.2719E−128 |
| Hspa8 | NA | NA | 4.30569E−21 | NA |
| Serping1 | 0.535830748 | NA | NA | 3.5363E−179 |
| Rgs16 | NA | 3.5457E−188 | NA | NA |
| Vtn | 0.516935534 | NA | NA | 8.9644E−294 |
| Gm2a | NA | 6.60916E−93 | NA | NA |
| Uroc1 | 0.459322934 | 1.13457E−82 | NA | 3.99387E−76 |
| Snord13 | NA | NA | 0.000327047 | NA |
| Akr1d1 | NA | NA | 0.00030931 | NA |
| Mgll | NA | 6.615E−101 | NA | NA |
| Fah | 0.325518495 | NA | NA | 1.50517E−71 |
| Mttp | NA | 2.14293E−79 | NA | NA |
| Eci2 | NA | 5.23022E−88 | NA | NA |
| Igf1 | NA | 2.85872E−86 | NA | NA |
| C4b | 0.820022988 | 4.29867E−67 | 7.45309E−90 | 0 |
| F9 | 0.303980935 | NA | NA | 1.74064E−49 |
| Sardh | 0.28626336 | NA | NA | 2.96273E−45 |
| F5 | 0.436709177 | NA | NA | 1.06285E−75 |
| Lpin1 | 0.382955017 | 0 | NA | 4.53406E−64 |
| Glul | 0.495360357 | 1.0077E−186 | 1 | 0.000261488 |

Albumin is the most abundant serum protein and is produced by all hepatocytes across the liver lobule, with the highest expression in the periportal region. Acute injury in both models resulted in a dramatic upregulation of albumin across the entire liver lobule beginning at the earliest observed time points (A6 and P3) (FIG. 20E). However, select genes involved in essential liver function responded at a level correlative to the extent of injury (F2 and Pck1). This is consistent with the larger total loss of hepatocytes in the PH model compared to the APAP model (˜70% vs ˜10%, respectively), resulting in a greater need for functional compensation. Applicants observed a dramatic up-regulation in metallothionein (Mt1) in both injury models (FIG. 20E). It has been suggested that Mt1 may serve two purposes in tissue injury: protection against further oxidative damage and support for the proliferative response²⁷. Further, Mt1 has previously been shown to be upregulated in the liver following PH^{28,29}. Mt1 was upregulated in all hepatocytes across the lobule and to a greater degree in PH than APAP. It remained elevated throughout the PH time course, where an increased proliferative demand is present due to increased tissue loss. These findings highlight not only the rapidity of hepatic functional adaptation but also the plasticity of hepatocytes across the liver lobule.

Cell Proliferation is Inversely Correlated with Functional Adaptation

Liver regeneration research has traditionally focused on hepatocyte proliferation⁴⁻⁷. It is unknown whether actively dividing hepatocytes can equally contribute to functional compensation. Applicants observed a down-regulation of many hepatic function genes during activation of the proliferative response (A24, P48, FIG. 20E). Therefore, Applicants identified cells that became transcriptionally active for cell cycle genes in the scRNA-Seq dataset (FIG. 21A), and analyzed hepatocyte-specific transcript output compared to those cells at all time points that are not cycling. Compared to non-cycling cells (NC), there was a significant down-regulation of the Hepatocyte Signature Score in cycling cells (CC) in both injury conditions (FIG. 21B, 21C, FIG. 29, Methods). DEG revealed substantial differences between cycling and non-cycling cells. CCs expressed many classic cell proliferation markers and exhibited down-regulation of many hepatic function genes (FIG. 21D, Table 16). Proliferating hepatocytes in general scored lower for hepatocyte markers. This was corroborated in individual cells by smFISH for the glucose transporter Slc2a2 and immunofluorescent co-staining for proliferating cell nuclear antigen (PCNA, FIG. 21E, 21F), as well as mathematical modeling of the correlation of gene expression and PCNA protein levels (FIG. 21G, Methods). Other genes, such as Alb, did not appreciably change in NC vs. CC populations (FIG. 21E-21G). Taken together, these data suggest that proliferating hepatocytes have the ability to maintain expression of select hepatic functional markers, while many other hepatic genes appear to be compensated predominantly by non-proliferating hepatocytes.

TABLE 16
DEG for each treatment between cycling cells vs noncycling cells (CC vs NC).
Positive log fold-change (avg_logFC) values indicate genes up-regulated
in the CCs, negative values indicate genes up-regulated in the NCs. Calculated
using the FindMarkers function in the R package Seurat using the Wilcox test.

| ID | p_val | avg_logFC | pct.1 | pct.2 | p_val_adj |
|---|---|---|---|---|---|
| D17H6S56E-5 | 8.62E−39 | 1.669915133 | 0.747 | 0.057 | 2.14E−34 |
| Top2a | 3.79E−30 | 1.423669967 | 0.586 | 0.023 | 9.42E−26 |
| Esco2 | 1.69E−28 | 1.063954263 | 0.563 | 0.023 | 4.19E−24 |
| Cdca3 | 1.87E−28 | 1.007478565 | 0.598 | 0.04 | 4.64E−24 |
| Birc5 | 2.19E−27 | 0.939238952 | 0.552 | 0.023 | 5.43E−23 |
| Tubb5 | 1.64E−26 | 1.236380405 | 0.741 | 0.27 | 4.07E−22 |
| Hmmr | 1.68E−26 | 1.114822336 | 0.511 | 0.006 | 4.17E−22 |
| Cdk1 | 6.10E−26 | 0.983632101 | 0.517 | 0.017 | 1.52E−21 |
| Prc1 | 2.43E−25 | 0.996936039 | 0.494 | 0.006 | 6.04E−21 |
| Ect2 | 5.32E−25 | 0.886912155 | 0.489 | 0.006 | 1.32E−20 |
| Tubb4b | 1.44E−24 | 1.145456059 | 0.753 | 0.322 | 3.56E−20 |
| Mki67 | 2.01E−24 | 1.416251498 | 0.523 | 0.034 | 4.99E−20 |
| Smc4 | 3.92E−23 | 1.307409004 | 0.649 | 0.195 | 9.74E−19 |
| Rrm2 | 9.27E−22 | 0.889506091 | 0.5 | 0.046 | 2.30E−17 |
| Smc2 | 2.33E−21 | 0.902524002 | 0.477 | 0.034 | 5.78E−17 |
| Cdc20 | 3.48E−21 | 1.087349379 | 0.454 | 0.023 | 8.65E−17 |
| Tpx2 | 7.77E−21 | 0.859308828 | 0.431 | 0.011 | 1.93E−16 |
| Ckap21 | 8.84E−21 | 0.876202937 | 0.408 | 0 | 2.20E−16 |
| Ckap2 | 1.88E−20 | 0.747241586 | 0.402 | 0 | 4.68E−16 |
| Ccna2 | 1.71E−19 | 0.604307576 | 0.397 | 0.006 | 4.24E−15 |
| Aurka | 3.03E−19 | 0.703458561 | 0.391 | 0.006 | 7.51E−15 |
| Spc25 | 3.03E−19 | 0.817339295 | 0.471 | 0.057 | 7.53E−15 |
| Cenpf | 8.07E−19 | 1.039724096 | 0.402 | 0.017 | 2.00E−14 |
| Pbk | 1.10E−18 | 0.591264838 | 0.385 | 0.006 | 2.72E−14 |
| Spc24 | 2.45E−18 | 0.770336501 | 0.534 | 0.126 | 6.09E−14 |
| Nek2 | 4.81E−18 | 0.722477841 | 0.379 | 0.011 | 1.19E−13 |
| Knstm | 1.04E−16 | 0.718716577 | 0.356 | 0.011 | 2.57E−12 |
| Kif20b | 1.19E−16 | 0.714194384 | 0.333 | 0 | 2.96E−12 |
| Nuf2 | 1.19E−16 | 0.537828732 | 0.333 | 0 | 2.96E−12 |
| Nusap1 | 2.41E−16 | 0.528310034 | 0.328 | 0 | 5.98E−12 |
| Racgap1 | 2.58E−16 | 0.493496977 | 0.339 | 0.006 | 6.42E−12 |
| Nucks1 | 3.01E−16 | 0.858143103 | 0.736 | 0.443 | 7.46E−12 |
| Kif20a | 4.27E−16 | 0.68044045 | 0.379 | 0.029 | 1.06E−11 |
| RP23-45G16.5 | 5.85E−16 | 0.702310221 | 0.385 | 0.034 | 1.45E−11 |
| Ube2c | 1.66E−15 | 0.722670005 | 0.345 | 0.017 | 4.12E−11 |
| Nudt7 | 3.26E−15 | −0.78138871 | 0.552 | 0.793 | 8.11E−11 |
| Cenpa | 3.29E−15 | 0.567785956 | 0.391 | 0.04 | 8.16E−11 |
| Ccnb2 | 9.67E−15 | 0.616222031 | 0.379 | 0.04 | 2.40E−10 |
| Anln | 1.14E−14 | 0.635712412 | 0.322 | 0.011 | 2.83E−10 |
| Prim1 | 1.26E−14 | 0.631016973 | 0.414 | 0.069 | 3.12E−10 |
| Rrm1 | 1.42E−14 | 0.677696452 | 0.391 | 0.052 | 3.52E−10 |
| Kif11 | 2.77E−14 | 0.564607575 | 0.31 | 0.011 | 6.87E−10 |
| Cdkn1a | 4.16E−14 | 0.758583248 | 0.5 | 0.155 | 1.03E−09 |
| C330027C09Rik | 6.06E−14 | 0.591386211 | 0.368 | 0.046 | 1.51E−09 |
| Tubb6 | 6.78E−14 | 0.681201202 | 0.408 | 0.075 | 1.68E−09 |
| Ncapg | 9.52E−14 | 0.473229133 | 0.316 | 0.017 | 2.36E−09 |
| Tuba1b | 9.63E−14 | 0.557598251 | 0.425 | 0.086 | 2.39E−09 |
| Tacc3 | 9.94E−14 | 0.475936269 | 0.339 | 0.029 | 2.47E−09 |
| Cenpq | 1.16E−13 | 0.525048378 | 0.333 | 0.029 | 2.87E−09 |
| Ezh2 | 1.92E−13 | 0.548750728 | 0.356 | 0.046 | 4.78E−09 |
| Arl6ip1 | 1.93E−13 | 0.911516088 | 0.799 | 0.649 | 4.79E−09 |
| Cdca8 | 3.66E−13 | 0.503278989 | 0.293 | 0.011 | 9.09E−09 |
| Kif4 | 4.33E−13 | 0.434892938 | 0.264 | 0 | 1.07E−08 |
| Dbf4 | 5.83E−13 | 0.561185724 | 0.351 | 0.046 | 1.45E−08 |
| Cenpe | 6.54E−13 | 0.655143344 | 0.287 | 0.011 | 1.63E−08 |
| Arhgap11a | 8.37E−13 | 0.391036423 | 0.259 | 0 | 2.08E−08 |
| Pcna | 8.96E−13 | 0.612264476 | 0.414 | 0.092 | 2.23E−08 |
| Mcm5 | 1.48E−12 | 0.50726949 | 0.31 | 0.029 | 3.67E−08 |
| Map4k4 | 2.73E−12 | 0.558455532 | 0.305 | 0.029 | 6.78E−08 |
| Aurkb | 5.93E−12 | 0.413096395 | 0.241 | 0 | 1.47E−07 |
| Ttk | 5.93E−12 | 0.390521577 | 0.241 | 0 | 1.47E−07 |
| Plk1 | 6.03E−12 | 0.404527572 | 0.264 | 0.011 | 1.50E−07 |
| Diap3 | 7.26E−12 | 0.400030606 | 0.264 | 0.011 | 1.80E−07 |
| Incenp | 8.17E−12 | 0.536649645 | 0.276 | 0.017 | 2.03E−07 |
| Apoa5 | 8.37E−12 | −0.482783443 | 0.759 | 0.931 | 2.08E−07 |
| Neurl1b | 8.62E−12 | 0.423286963 | 0.253 | 0.006 | 2.14E−07 |
| Uox | 9.44E−12 | −0.499476519 | 0.856 | 0.954 | 2.34E−07 |
| Shcbp1 | 1.13E−11 | 0.376352181 | 0.236 | 0 | 2.81E−07 |
| 2700094K13Rik | 1.52E−11 | 0.548631981 | 0.448 | 0.138 | 3.79E−07 |
| Calm2 | 1.76E−11 | 0.710664504 | 0.695 | 0.46 | 4.36E−07 |
| Kif2c | 2.15E−11 | 0.404282153 | 0.23 | 0 | 5.34E−07 |
| Mcm6 | 3.26E−11 | 0.512710004 | 0.316 | 0.046 | 8.09E−07 |
| Aox3 | 3.32E−11 | −0.684717258 | 0.632 | 0.851 | 8.23E−07 |
| Ccnb1 | 3.71E−11 | 0.363013517 | 0.241 | 0.006 | 9.21E−07 |
| Car3 | 4.24E−11 | −0.588546974 | 0.695 | 0.897 | 1.05E−06 |
| Rad51 | 4.34E−11 | 0.316938048 | 0.259 | 0.017 | 1.08E−06 |
| Hells | 4.56E−11 | 0.404612434 | 0.247 | 0.011 | 1.13E−06 |
| Casc5 | 6.49E−11 | 0.511352815 | 0.236 | 0.006 | 1.61E−06 |
| Tyms | 8.41E−11 | 0.462105237 | 0.362 | 0.086 | 2.09E−06 |
| Foxm1 | 8.70E−11 | 0.333261782 | 0.23 | 0.006 | 2.16E−06 |
| Elovl3 | 1.52E−10 | −0.63437531 | 0.259 | 0.58 | 3.78E−06 |
| Zwilch | 1.70E−10 | 0.29872803 | 0.224 | 0.006 | 4.21E−06 |
| Ncapg2 | 2.25E−10 | 0.341013594 | 0.259 | 0.023 | 5.60E−06 |
| Cdca2 | 2.72E−10 | 0.308760405 | 0.207 | 0 | 6.75E−06 |
| Mad2l1 | 3.19E−10 | 0.421535724 | 0.276 | 0.034 | 7.93E−06 |
| Mnrnpab | 3.20E−10 | 0.613103641 | 0.713 | 0.523 | 7.95E−06 |
| Cenpm | 4.51E−10 | 0.356059134 | 0.293 | 0.046 | 1.12E−05 |
| Tuba1c | 4.87E−10 | 0.741932397 | 0.644 | 0.42 | 1.21E−05 |
| Tdo2 | 5.03E−10 | −0.43203435 | 0.856 | 0.943 | 1.25E−05 |
| Serpinb8 | 8.51E−10 | 0.37625294 | 0.236 | 0.017 | 2.11E−05 |
| Abcb11 | 9.09E−10 | −0.548869051 | 0.603 | 0.816 | 2.26E−05 |
| Stmn1 | 9.54E−10 | 0.25333229 | 0.224 | 0.011 | 2.37E−05 |
| Hsd3b5 | 1.12E−09 | −0.663150283 | 0.184 | 0.466 | 2.79E−05 |
| Kif22 | 1.28E−09 | 0.326233499 | 0.218 | 0.011 | 3.17E−05 |
| Cdkn2c | 1.29E−09 | 0.414018009 | 0.282 | 0.046 | 3.21E−05 |
| Hjurp | 1.34E−09 | 0.746962345 | 0.517 | 0.253 | 3.33E−05 |
| Rad21 | 1.71E−09 | 0.693598694 | 0.506 | 0.236 | 4.24E−05 |
| Plk4 | 1.79E−09 | 0.445870522 | 0.241 | 0.023 | 4.45E−05 |
| Hal | 1.84E−09 | −0.904034035 | 0.408 | 0.603 | 4.58E−05 |
| Cps1 | 2.12E−09 | −0.449845949 | 0.931 | 0.96 | 5.26E−05 |
| Atad2 | 2.89E−09 | 0.538790709 | 0.247 | 0.029 | 7.18E−05 |
| Oip5 | 3.27E−09 | 0.203934231 | 0.184 | 0 | 8.11E−05 |
| 2810408I11Rik | 3.72E−09 | 0.364202374 | 0.276 | 0.046 | 9.23E−05 |
| Apoa1 | 4.10E−09 | −0.266547284 | 1 | 1 | 0.00010193 |
| Ncapd2 | 4.39E−09 | 0.272663933 | 0.207 | 0.011 | 0.000109071 |
| Lmnb1 | 5.28E−09 | 0.355776415 | 0.241 | 0.029 | 0.000131052 |
| Rbp4 | 5.80E−09 | −0.27547877 | 0.966 | 0.994 | 0.000144054 |
| Sgol2 | 6.04E−09 | 0.41927217 | 0.178 | 0 | 0.000149884 |
| Slfn9 | 6.27E−09 | 0.443882082 | 0.23 | 0.023 | 0.000155728 |
| Thrsp | 6.81E−09 | −0.571637606 | 0.379 | 0.621 | 0.000169221 |
| Tmpo | 9.73E−09 | 0.620801056 | 0.506 | 0.253 | 0.000241635 |
| Vars | 1.02E−08 | 0.45501932 | 0.391 | 0.132 | 0.000252944 |
| Mbl2 | 1.03E−08 | 0.510840856 | 0.822 | 0.736 | 0.000255976 |
| H2afx | 1.34E−08 | 0.293587722 | 0.224 | 0.023 | 0.000331923 |
| Lig1 | 1.35E−08 | 0.399890983 | 0.224 | 0.023 | 0.000335561 |
| Mdc1 | 1.60E−08 | 0.349180142 | 0.282 | 0.057 | 0.000398491 |
| Aspm | 1.65E−08 | 0.327831807 | 0.184 | 0.006 | 0.000409937 |
| Dlgap5 | 1.82E−08 | 0.324966932 | 0.184 | 0.006 | 0.000451854 |
| Arhgef39 | 2.04E−08 | 0.20651116 | 0.167 | 0 | 0.000507222 |
| Fabp1 | 2.31E−08 | −0.329852078 | 0.977 | 0.989 | 0.000574129 |
| Ncaph | 2.38E−08 | 0.319017176 | 0.207 | 0.017 | 0.000591378 |
| Hist1h1b | 2.74E−08 | 0.375177572 | 0.178 | 0.006 | 0.000680526 |
| Hat1 | 2.94E−08 | 0.350598047 | 0.287 | 0.069 | 0.000731007 |
| Dnajc9 | 3.45E−08 | 0.43096565 | 0.31 | 0.08 | 0.000855729 |
| Fen1 | 3.71E−08 | 0.324876638 | 0.224 | 0.029 | 0.000922489 |
| Prr11 | 3.74E−08 | 0.242134134 | 0.161 | 0 | 0.000929068 |
| Ska1 | 3.74E−08 | 0.231568321 | 0.161 | 0 | 0.000929068 |
| Ywhah | 4.02E−08 | 0.354504609 | 0.293 | 0.069 | 0.000998691 |
| Pecr | 4.99E−08 | −0.394577277 | 0.448 | 0.69 | 0.001239461 |
| Cdkn2d | 5.42E−08 | 0.258022749 | 0.218 | 0.029 | 0.001344828 |
| Bub1 | 5.43E−08 | 0.222527368 | 0.195 | 0.017 | 0.001347871 |
| Comt | 5.64E−08 | −0.336939912 | 0.81 | 0.943 | 0.001399472 |
| Mlf1ip | 5.65E−08 | 0.29860168 | 0.172 | 0.006 | 0.00140177 |
| Psip1 | 5.68E−08 | 0.412140435 | 0.402 | 0.155 | 0.001410087 |
| Ccdc34 | 6.19E−08 | 0.450221698 | 0.27 | 0.063 | 0.001537453 |
| Haus4 | 6.51E−08 | 0.312841092 | 0.287 | 0.069 | 0.001616969 |
| Alb | 6.61E−08 | −0.297392831 | 1 | 1 | 0.00164176 |
| Mastl | 6.79E−08 | 0.369179893 | 0.207 | 0.023 | 0.001686689 |
| Mxd3 | 6.83E−08 | 0.223725416 | 0.155 | 0 | 0.001696992 |
| Ndc80 | 8.36E−08 | 0.276132253 | 0.167 | 0.006 | 0.002076373 |
| Gpx7 | 8.67E−08 | 0.222864148 | 0.184 | 0.011 | 0.00215394 |
| Bhmt | 8.84E−08 | −0.436626346 | 0.828 | 0.908 | 0.002196332 |
| Hn1 | 1.11E−07 | 0.35302193 | 0.293 | 0.08 | 0.002746119 |
| Gins2 | 1.21E−07 | 0.259574009 | 0.224 | 0.034 | 0.002996965 |
| Tcf19 | 1.48E−07 | 0.294242686 | 0.195 | 0.023 | 0.003684451 |
| Mcm8 | 1.52E−07 | 0.207497457 | 0.161 | 0.006 | 0.003768811 |
| Dtymk | 1.55E−07 | 0.428448179 | 0.414 | 0.178 | 0.003845818 |
| Kpnb1 | 1.58E−07 | 0.642471426 | 0.598 | 0.374 | 0.003919368 |
| Rangap1 | 1.75E−07 | 0.497310257 | 0.54 | 0.333 | 0.004336791 |
| Klf6 | 1.77E−07 | 0.416981155 | 0.287 | 0.075 | 0.004404128 |
| Ces3b | 1.89E−07 | −0.468451514 | 0.368 | 0.586 | 0.00469596 |
| Cml2 | 1.90E−07 | −0.420806129 | 0.741 | 0.885 | 0.00472796 |
| Cdkn3 | 1.92E−07 | 0.294475986 | 0.207 | 0.029 | 0.004766244 |
| 4632434I11Rik | 1.99E−07 | 0.219596415 | 0.201 | 0.023 | 0.004939952 |
| Fignl1 | 2.26E−07 | 0.208834541 | 0.144 | 0 | 0.005615642 |
| Rbl1 | 2.74E−07 | 0.288971872 | 0.213 | 0.034 | 0.006797795 |
| Hpd | 2.75E−07 | −0.352708023 | 0.902 | 0.931 | 0.00683379 |
| Selenbp2 | 2.82E−07 | −0.546009344 | 0.632 | 0.787 | 0.006995798 |
| Tk1 | 2.90E−07 | 0.36336783 | 0.339 | 0.121 | 0.007199845 |
| Bub1b | 3.10E−07 | 0.254260074 | 0.155 | 0.006 | 0.007691412 |
| Chaf1a | 3.45E−07 | 0.231375382 | 0.178 | 0.017 | 0.008567106 |
| Sae1 | 3.47E−07 | 0.394616101 | 0.437 | 0.195 | 0.008620339 |
| Pole3 | 4.32E−07 | 0.360643954 | 0.402 | 0.172 | 0.01072822 |
| Ccl9 | 4.40E−07 | 0.66722565 | 0.655 | 0.448 | 0.010924443 |
| Cdk5rap2 | 4.62E−07 | 0.331940452 | 0.201 | 0.029 | 0.011466687 |
| Scp2 | 4.81E−07 | −0.331704281 | 0.862 | 0.954 | 0.011953871 |
| Suv39h1 | 4.94E−07 | 0.290883863 | 0.178 | 0.017 | 0.012257487 |
| Mcm7 | 5.46E−07 | 0.361201441 | 0.276 | 0.08 | 0.013549576 |
| Slco1a1 | 5.51E−07 | −0.459298123 | 0.477 | 0.678 | 0.013677968 |
| Gmnn | 6.82E−07 | 0.35501373 | 0.276 | 0.08 | 0.016938424 |
| Mnrnpa2b1 | 6.92E−07 | 0.364373341 | 0.799 | 0.73 | 0.017192749 |
| mt-Co1 | 7.02E−07 | −0.448010334 | 0.925 | 0.954 | 0.017435262 |
| Reep4 | 7.73E−07 | 0.301442648 | 0.23 | 0.052 | 0.019199816 |
| Hsd17b2 | 7.95E−07 | −0.452371152 | 0.523 | 0.695 | 0.019736346 |
| Upp2 | 8.61E−07 | −0.509440895 | 0.385 | 0.575 | 0.021384944 |
| Hsd17b6 | 8.67E−07 | −0.508243085 | 0.149 | 0.368 | 0.021524829 |
| Bub3 | 9.18E−07 | 0.410061599 | 0.368 | 0.155 | 0.022791598 |
| Pdcd4 | 9.47E−07 | −0 480756367 | 0 374 | 0 575 | 0 023527462 |
| Dek | 9.93E−07 | 0.565483667 | 0.523 | 0.322 | 0.02464934 |
| Tubb2a | 1.08E−06 | 0.465591059 | 0.586 | 0.374 | 0.026786812 |
| 2810007J24Rik | 1.10E−06 | −0.446441418 | 0.632 | 0.793 | 0.027231326 |
| Rpa1 | 1.13E−06 | 0.370621427 | 0.391 | 0.184 | 0.028159865 |
| Ranbp1 | 1.16E−06 | 0.446858539 | 0.466 | 0.241 | 0.02882351 |
| Hsp90aa1 | 1.18E−06 | 0.438704989 | 0.483 | 0.27 | 0.029289082 |
| Eri2 | 1.25E−06 | 0.236618768 | 0.19 | 0.029 | 0.030953592 |
| Hyls1 | 1.34E−06 | 0.272967156 | 0.126 | 0 | 0.033156502 |
| Insig2 | 1.34E−06 | −0.483909727 | 0.5 | 0.672 | 0.033305631 |
| Dhfr | 1.49E−06 | 0.423679824 | 0.489 | 0.287 | 0.037008278 |
| Mybl1 | 1.52E−06 | 0.287881605 | 0.167 | 0.017 | 0.037682685 |
| Fam111a | 1.72E−06 | 0.325832889 | 0.138 | 0.006 | 0.042742649 |
| Cenpk | 1.80E−06 | 0.202635563 | 0.149 | 0.011 | 0.044595626 |
| Mthfd2 | 2.03E−06 | 0.219619521 | 0.149 | 0.011 | 0.050442715 |
| Ndc1 | 2.13E−06 | 0.296534986 | 0.27 | 0.08 | 0.052968483 |
| Etnppl | 2.18E−06 | −0.484390252 | 0.178 | 0.379 | 0.054221467 |
| Nop58 | 2.23E−06 | 0.433692928 | 0.385 | 0.178 | 0.055321793 |
| Rfc4 | 2.26E−06 | 0.237784064 | 0.218 | 0.046 | 0.056232628 |
| Mup3 | 2.36E−06 | −0.30602015 | 1 | 1 | 0.058588809 |
| Trim59 | 2.40E−06 | 0.229039212 | 0.121 | 0 | 0.059637901 |
| Rad18 | 2.51E−06 | 0.261961421 | 0.161 | 0.017 | 0.062371563 |
| Cdo1 | 2.72E−06 | −0.330478369 | 0.833 | 0.943 | 0.067429661 |
| Timm50 | 2.74E−06 | 0.385094148 | 0.408 | 0.201 | 0.068009274 |
| Anp32e | 2.86E−06 | 0.300941339 | 0.351 | 0.149 | 0.071040964 |
| Pck1 | 2.96E−06 | −0.348158569 | 0.92 | 0.948 | 0.073458089 |
| Reep6 | 3.09E−06 | −0.370256216 | 0.621 | 0.782 | 0.076699226 |
| Lin54 | 3.11E−06 | 0.260601036 | 0.218 | 0.052 | 0.077314843 |
| Xpo1 | 3.11E−06 | 0.45065952 | 0.397 | 0.19 | 0.077346963 |
| Haus6 | 3.17E−06 | 0.287954493 | 0.167 | 0.023 | 0.078636347 |
| Eri1 | 3.53E−06 | 0.374929132 | 0.333 | 0.132 | 0.087545947 |
| Gas2l3 | 3.85E−06 | 0.301751089 | 0.253 | 0.075 | 0.095490635 |
| Dut | 3.86E−06 | 0.302722263 | 0.293 | 0.103 | 0.095759296 |
| E2f8 | 4.41E−06 | 0.273383207 | 0.184 | 0.034 | 0.109423351 |
| Rnaseh2c | 4.74E−06 | 0.395665408 | 0.443 | 0.224 | 0.11774774 |
| C8a | 4.93E−06 | −0.348005456 | 0.782 | 0.914 | 0.122504367 |
| Gins1 | 5.06E−06 | 0.225027831 | 0.19 | 0.034 | 0.12576907 |
| Ran | 5.35E−06 | 0.442539115 | 0.414 | 0.218 | 0.132864759 |
| mt-Nd2 | 5.70E−06 | −0.339042127 | 0.954 | 0.971 | 0.141510802 |
| Phf5a | 5.97E−06 | 0.330461091 | 0.385 | 0.184 | 0.148154476 |
| Cdkn1b | 6.08E−06 | 0.392627086 | 0.356 | 0.161 | 0.150875433 |
| Gnmt | 6.34E−06 | −0.235276025 | 0.977 | 0.977 | 0.157376056 |
| Atpif1 | 6.97E−06 | 0.400342706 | 0.385 | 0.19 | 0.173069233 |
| Glud1 | 7.38E−06 | −0.282143773 | 0.891 | 0.977 | 0.183294842 |
| Cyp2f2 | 7.51E−06 | −0.612374975 | 0.471 | 0.644 | 0.186415303 |
| Mis18bp1 | 7.72E−06 | 0.227536238 | 0.109 | 0 | 0.191659469 |
| Nudc | 8.23E−06 | 0.211503644 | 0.345 | 0.144 | 0.204357248 |
| Setd8 | 9.03E−06 | 0.375290352 | 0.414 | 0.224 | 0.22415728 |
| Serpina3n | 9.17E−06 | 0.394742592 | 0.828 | 0.77 | 0.227701048 |
| Tgm2 | 1.03E−05 | 0.459985731 | 0.529 | 0.345 | 0.25578588 |
| Serpina1b | 1.11E−05 | −0.20979262 | 0.966 | 0.971 | 0.276483244 |
| Ybx1 | 1.12E−05 | 0.433858711 | 0.667 | 0.489 | 0.278101738 |
| Scd1 | 1.14E−05 | −0.503565027 | 0.856 | 0.931 | 0.282777037 |
| Rpa2 | 1.18E−05 | 0.224823858 | 0.218 | 0.057 | 0.292962164 |
| Kmo | 1.24E−05 | 0.384931727 | 0.799 | 0.724 | 0.308137573 |
| H2afv | 1.25E−05 | 0.317443687 | 0.437 | 0.23 | 0.309287953 |
| Cml1 | 1.25E−05 | −0.406306558 | 0.391 | 0.592 | 0.311564113 |
| Pagr1a | 1.32E−05 | 0.277205013 | 0.224 | 0.063 | 0.327068434 |
| Slc38a4 | 1.32E−05 | −0.312113482 | 0.753 | 0.862 | 0.327971723 |
| Srsf3 | 1.36E−05 | 0.336110854 | 0.385 | 0.201 | 0.337484189 |
| Rps20 | 1.37E−05 | 0.382765037 | 0.667 | 0.494 | 0.339399222 |
| Ublcp1 | 1.39E−05 | 0.274341853 | 0.23 | 0.069 | 0.346164244 |
| Malat1 | 1.41E−05 | −0.80879767 | 0.448 | 0.667 | 0.349428979 |
| Snx7 | 1.46E−05 | 0.316271647 | 0.379 | 0.178 | 0.361577964 |
| Wbp5 | 1.51E−05 | 0.284833036 | 0.333 | 0.149 | 0.375816193 |
| Rfc5 | 1.55E−05 | 0.248445785 | 0.247 | 0.08 | 0.38412716 |
| Haao | 1.57E−05 | −0.31181035 | 0.695 | 0.828 | 0.388664443 |
| Pigr | 1.67E−05 | −0.440084062 | 0.787 | 0.845 | 0.414812064 |
| Ckap5 | 1.69E−05 | 0.217434157 | 0.259 | 0.086 | 0.420108397 |
| Txnrd1 | 1.73E−05 | 0.401234639 | 0.655 | 0.529 | 0.429420356 |
| Vrk1 | 1.74E−05 | 0.274134129 | 0.172 | 0.034 | 0.4323753 |
| Slc38a3 | 1.74E−05 | −0.361188229 | 0.701 | 0.851 | 0.432795193 |
| G3bp1 | 1.78E−05 | 0.375563886 | 0.362 | 0.167 | 0.441399057 |
| Cpn2 | 1.97E−05 | −0.344002539 | 0.483 | 0.678 | 0.489125563 |
| Bhmt2 | 2.08E−05 | −0.37069517 | 0.443 | 0.609 | 0.516393709 |
| Hspa8 | 2.37E−05 | 0.340473403 | 0.77 | 0.684 | 0.589258722 |
| 7-Sep | 2.38E−05 | 0.455670864 | 0.517 | 0.316 | 0.589819559 |
| Rfc2 | 2.45E−05 | 0.360830553 | 0.31 | 0.138 | 0.608206199 |
| Ppil1 | 2.45E−05 | 0.227042373 | 0.259 | 0.092 | 0.608514071 |
| Dctpp1 | 2.58E−05 | 0.35767839 | 0.397 | 0.207 | 0.640693109 |
| Mcm2 | 2.64E−05 | 0.285279656 | 0.19 | 0.046 | 0.654386851 |
| Rpn2 | 2.71E−05 | 0.405358032 | 0.569 | 0.408 | 0.67281896 |
| Ube2k | 2.83E−05 | 0.348055395 | 0.517 | 0.31 | 0.703678327 |
| Mbd1 | 2.87E−05 | 0.364824514 | 0.333 | 0.149 | 0.713355213 |
| Ndrg2 | 3.23E−05 | −0.296808072 | 0.868 | 0.931 | 0.80182229 |
| Srxn1 | 3.67E−05 | 0.319778489 | 0.379 | 0.195 | 0.912373853 |
| Calm3 | 3.76E−05 | 0.432242817 | 0.385 | 0.213 | 0.933445302 |
| Rnaseh2b | 4.08E−05 | 0.217676579 | 0.213 | 0.063 | 1 |
| Mat2a | 4.11E−05 | 0.482635376 | 0.471 | 0.305 | 1 |
| Atf3 | 4.15E−05 | 0.362943833 | 0.425 | 0.218 | 1 |
| Nfatc2ip | 4.47E−05 | 0.200297983 | 0.132 | 0.017 | 1 |
| Gpd1 | 4.48E−05 | −0.29447894 | 0.466 | 0.649 | 1 |
| G2e3 | 4.53E−05 | 0.272666918 | 0.201 | 0.057 | 1 |
| Fgg | 4.69E−05 | −0.203445263 | 0.994 | 1 | 1 |
| Mlec | 4.74E−05 | 0.490588829 | 0.621 | 0.483 | 1 |
| Ces2a | 4.90E−05 | −0.273049604 | 0.592 | 0.787 | 1 |
| Lap3 | 5.10E−05 | −0.274273748 | 0.638 | 0.782 | 1 |
| Tmem53 | 5.34E−05 | 0.281369697 | 0.339 | 0.161 | 1 |
| Asl | 5.78E−05 | −0.383514121 | 0.603 | 0.776 | 1 |
| Tcp1 | 6.52E−05 | 0.381115369 | 0.621 | 0.477 | 1 |
| Brca2 | 6.77E−05 | 0.311589577 | 0.138 | 0.023 | 1 |
| Otc | 6.95E−05 | −0.283470665 | 0.805 | 0.868 | 1 |
| Sds | 7.30E−05 | −0.536793811 | 0.247 | 0.408 | 1 |
| Snrpd1 | 7.67E−05 | 0.27940919 | 0.282 | 0.121 | 1 |
| Prtn3 | 7.78E−05 | 0.284555077 | 0.27 | 0.109 | 1 |
| Hp1bp3 | 7.93E−05 | 0.466899479 | 0.511 | 0.351 | 1 |
| Cat | 7.96E−05 | −0.248150835 | 0.925 | 0.948 | 1 |
| Snrpa1 | 7.97E−05 | 0.263512181 | 0.305 | 0.138 | 1 |
| Hn1l | 8.32E−05 | 0.226911148 | 0.299 | 0.132 | 1 |
| Egfr | 8.33E−05 | −0.261805449 | 0.81 | 0.862 | 1 |
| Pcnt | 8.62E−05 | 0.226629309 | 0.149 | 0.029 | 1 |
| Pgp | 8.70E−05 | 0.275068866 | 0.374 | 0.207 | 1 |
| Ldha | 8.93E−05 | 0.297270276 | 0.891 | 0.851 | 1 |
| Gstm3 | 9.25E−05 | 0.669387901 | 0.598 | 0.42 | 1 |
| Ccnf | 9.40E−05 | 0.268249638 | 0.253 | 0.098 | 1 |
| Hmgcs2 | 9.40E−05 | −0.266526208 | 0.868 | 0.914 | 1 |
| Ccdc77 | 9.48E−05 | 0.286413022 | 0.201 | 0.063 | 1 |
| Cbx5 | 0.000100141 | 0.36710943 | 0.356 | 0.19 | 1 |
| Tgoln1 | 0.000102696 | −0.325934331 | 0.414 | 0.569 | 1 |
| Rps11 | 0.000102835 | 0.365440855 | 0.489 | 0.316 | 1 |
| Mdh1 | 0.000104647 | −0.290065525 | 0.747 | 0.874 | 1 |
| Nubp1 | 0.000105157 | 0.381850948 | 0.379 | 0.213 | 1 |
| F7 | 0.000108949 | −0.400251784 | 0.425 | 0.569 | 1 |
| Smad4 | 0.000111273 | 0.253879461 | 0.351 | 0.172 | 1 |
| Adck3 | 0.000114764 | −0.374339709 | 0.54 | 0.638 | 1 |
| Tprkb | 0.000117286 | −0.299935577 | 0.339 | 0.506 | 1 |
| Slc25a48 | 0.000117538 | 0.381303403 | 0.253 | 0.103 | 1 |
| Hspa14 | 0.000117567 | 0.271589342 | 0.339 | 0.172 | 1 |
| Azgp1 | 0.000122515 | −0.242890214 | 0.92 | 0.971 | 1 |
| Tcirg1 | 0.000123612 | 0.200461262 | 0.27 | 0.109 | 1 |
| Cyp2d26 | 0.000125749 | −0.291556913 | 0.753 | 0.81 | 1 |
| Cenpl | 0.000131986 | 0.266357687 | 0.218 | 0.075 | 1 |
| Mug2 | 0.000132583 | −0.405169711 | 0.58 | 0.713 | 1 |
| Agxt | 0.000139728 | −0.39457087 | 0.529 | 0.661 | 1 |
| Mdm2 | 0.000153218 | 0.340634023 | 0.322 | 0.161 | 1 |
| Snrnp48 | 0.000154667 | 0.204158168 | 0.224 | 0.086 | 1 |
| Mrps6 | 0.000155642 | 0.310188333 | 0.253 | 0.109 | 1 |
| Nup205 | 0.000159713 | 0.261616453 | 0.213 | 0.075 | 1 |
| Aldh1l1 | 0.000160944 | −0.253939008 | 0.862 | 0.931 | 1 |
| Serpina1d | 0.000167571 | −0.259689069 | 0.868 | 0.943 | 1 |
| Emc8 | 0.000168202 | 0.231906188 | 0.333 | 0.167 | 1 |
| Ccng1 | 0.00016908 | 0.295124571 | 0.356 | 0.19 | 1 |
| Anp32b | 0.000176313 | 0.396891769 | 0.448 | 0.299 | 1 |
| Ykt6 | 0.000180442 | 0.380489397 | 0.351 | 0.195 | 1 |
| Nags | 0.000184693 | 0.338438646 | 0.466 | 0.282 | 1 |
| Slc27a2 | 0.000184889 | −0.252814105 | 0.845 | 0.92 | 1 |
| Gls2 | 0.000185814 | −0.326680339 | 0.402 | 0.586 | 1 |
| Hist1h1e | 0.000188497 | 0.244826085 | 0.224 | 0.08 | 1 |
| Afm | 0.000191908 | −0.290504337 | 0.466 | 0.621 | 1 |
| Zfp367 | 0.000193424 | 0.263894736 | 0.264 | 0.115 | 1 |
| Wfdc17 | 0.000208137 | 0.287356386 | 0.19 | 0.057 | 1 |
| Pola1 | 0.000213231 | 0.244320999 | 0.155 | 0.04 | 1 |
| Ptma | 0.000218547 | 0.21952298 | 0.27 | 0.121 | 1 |
| Lgals9 | 0.000228862 | −0.329391013 | 0.638 | 0.753 | 1 |
| mt-Cytb | 0.000232534 | −0.255568848 | 0.902 | 0.937 | 1 |
| Tkt | 0.000234475 | 0.344836871 | 0.552 | 0.402 | 1 |
| Ppp4c | 0.000234501 | 0.251294639 | 0.236 | 0.092 | 1 |
| Tmem97 | 0.000242512 | 0.400583107 | 0.46 | 0.299 | 1 |
| Apob | 0.000245863 | −0.299875775 | 0.977 | 0.966 | 1 |
| Tpr | 0.000251283 | 0.456623718 | 0.523 | 0.379 | 1 |
| Gm2788 | 0.000271859 | −0.211472801 | 0.092 | 0.23 | 1 |
| Scaf11 | 0.000279695 | 0.292403074 | 0.437 | 0.27 | 1 |
| Clcc1 | 0.000279736 | 0.28298206 | 0.379 | 0.213 | 1 |
| Csrp2 | 0.000298218 | 0.28424465 | 0.322 | 0.161 | 1 |
| Ccbl2 | 0.000299733 | −0.300791814 | 0.443 | 0.586 | 1 |
| Rgn | 0.000301617 | 0.20528053 | 0.931 | 0.77 | 1 |
| Psmb1 | 0.000307241 | 0.286652401 | 0.713 | 0.626 | 1 |
| Snx5 | 0.000307539 | 0.239409221 | 0.391 | 0.224 | 1 |
| Cd5l | 0.000313116 | 0.45974441 | 0.218 | 0.08 | 1 |
| Ptp4a2 | 0.000320524 | 0.310982553 | 0.632 | 0.5 | 1 |
| Rpl13a | 0.000327249 | 0.278346993 | 0.799 | 0.747 | 1 |
| Idh1 | 0.00033107 | −0.290981994 | 0.661 | 0.793 | 1 |
| Mcm3 | 0.000341799 | 0.235971207 | 0.172 | 0.052 | 1 |
| Exosc8 | 0.000344627 | 0.250463363 | 0.247 | 0.103 | 1 |
| Hgd | 0.000355827 | −0.254976188 | 0.644 | 0.81 | 1 |
| Pdik1l | 0.000355912 | 0.273144229 | 0.224 | 0.092 | 1 |
| Gsr | 0.000358394 | 0.23781591 | 0.489 | 0.316 | 1 |
| Atp6v1c1 | 0.00036526 | 0.347468443 | 0.345 | 0.195 | 1 |
| Lpin1 | 0.000399187 | −0.352223257 | 0.207 | 0.356 | 1 |
| Gjb2 | 0.000401614 | −0.280030843 | 0.546 | 0.678 | 1 |
| Cby1 | 0.000404829 | 0.217990914 | 0.195 | 0.069 | 1 |
| Ssx2ip | 0.000412834 | 0.214346928 | 0.207 | 0.08 | 1 |
| Rrbp1 | 0.000430781 | −0.271973566 | 0.73 | 0.793 | 1 |
| Arf6 | 0.000433644 | 0.313882002 | 0.397 | 0.236 | 1 |
| Dgat2 | 0.000435033 | −0.228770348 | 0.621 | 0.724 | 1 |
| Rbbp8 | 0.000438587 | 0.302400414 | 0.213 | 0.086 | 1 |
| Lsm2 | 0.000454397 | 0.249449387 | 0.241 | 0.103 | 1 |
| Vbp1 | 0.000456079 | 0.276953076 | 0.247 | 0.109 | 1 |
| Taf9 | 0.000463719 | 0.292774574 | 0.345 | 0.19 | 1 |
| Gclc | 0.000486647 | 0.358953864 | 0.724 | 0.632 | 1 |
| Fxn | 0.000498295 | 0.207127367 | 0.218 | 0.086 | 1 |
| Luc7l3 | 0.000504815 | 0.276290665 | 0.374 | 0.218 | 1 |
| Gstm1 | 0.000520109 | 0.330492518 | 0.879 | 0.897 | 1 |
| Psmc1 | 0.000526832 | 0.307490063 | 0.569 | 0.414 | 1 |
| Ilf3 | 0.000535316 | 0.223647749 | 0.282 | 0.132 | 1 |
| Kynu | 0.000551235 | −0.298905085 | 0.425 | 0.569 | 1 |
| Hrsp12 | 0.000553455 | −0.308816674 | 0.736 | 0.822 | 1 |
| Matr3 | 0.000554487 | 0.350299293 | 0.46 | 0.316 | 1 |
| Trp53inp1 | 0.000556941 | 0.362824706 | 0.529 | 0.402 | 1 |
| Chd1l | 0.000559277 | 0.265372607 | 0.236 | 0.103 | 1 |
| Rbbp7 | 0.000560518 | 0.231563045 | 0.402 | 0.241 | 1 |
| Sfpq | 0.000561829 | 0.38724728 | 0.437 | 0.282 | 1 |
| F10 | 0.00057882 | −0.207677398 | 0.822 | 0.902 | 1 |
| Nono | 0.000580189 | 0.242303202 | 0.293 | 0.149 | 1 |
| Mup7 | 0.00058141 | −0.321619611 | 0.943 | 0.977 | 1 |
| Lifr | 0.000581781 | −0.280666669 | 0.5 | 0.638 | 1 |
| Fam25c | 0.000599837 | 0.25164092 | 0.667 | 0.494 | 1 |
| Ephx1 | 0.000600839 | 0.280611043 | 0.724 | 0.644 | 1 |
| Anapc1 | 0.000607883 | 0.206380812 | 0.27 | 0.126 | 1 |
| Tinf2 | 0.000607904 | 0.20827297 | 0.172 | 0.057 | 1 |
| Pih1d1 | 0.000628092 | 0.273760415 | 0.339 | 0.184 | 1 |
| Abca8a | 0.000637377 | −0.260277819 | 0.149 | 0.287 | 1 |
| Hoga1 | 0.000663082 | −0.273858244 | 0.385 | 0.511 | 1 |
| Aldh1b1 | 0.0006771 | −0.249178685 | 0.046 | 0.155 | 1 |
| Lmna | 0.000679483 | 0.326326742 | 0.356 | 0.224 | 1 |
| Cnot6 | 0.00069247 | 0.343829212 | 0.402 | 0.264 | 1 |
| Ncaph2 | 0.000721239 | 0.275618301 | 0.443 | 0.293 | 1 |
| Lbr | 0.000745604 | 0.272446091 | 0.259 | 0.121 | 1 |
| Npm1 | 0.000758444 | 0.29098559 | 0.431 | 0.282 | 1 |
| Kpna4 | 0.000768146 | 0.283598161 | 0.333 | 0.19 | 1 |
| Ctr9 | 0.000769461 | 0.317614844 | 0.333 | 0.184 | 1 |
| Upb1 | 0.000782909 | −0.283983501 | 0.563 | 0.69 | 1 |
| Sephs2 | 0.000827697 | −0.259638362 | 0.753 | 0.845 | 1 |
| Ssrp1 | 0.000834114 | 0.398030517 | 0.477 | 0.322 | 1 |
| Slc2a2 | 0.000863816 | −0.319257104 | 0.27 | 0.414 | 1 |
| Mt1 | 0.000867867 | 0.30883204 | 0.77 | 0.649 | 1 |
| Mug1 | 0.000886085 | −0.252219139 | 0.908 | 0.92 | 1 |
| Serpina12 | 0.000904201 | −0.439014517 | 0.161 | 0.287 | 1 |
| 0610005C13Rik | 0.000950132 | −0.289522779 | 0.5 | 0.644 | 1 |
| D230025D16Rik | 0.000950291 | 0.31895727 | 0.305 | 0.167 | 1 |
| Naa50 | 0.000993554 | 0.352403593 | 0.517 | 0.385 | 1 |
| Kif1b | 0.001005797 | −0.322074636 | 0.316 | 0.466 | 1 |
| Snrnp70 | 0.001007289 | 0.231363362 | 0.414 | 0.247 | 1 |
| Psmd7 | 0.001016916 | 0.315581184 | 0.575 | 0.448 | 1 |
| Uspl1 | 0.001037787 | 0.25033896 | 0.224 | 0.098 | 1 |
| Cdc27 | 0.001041649 | 0.225305361 | 0.305 | 0.161 | 1 |
| Cpsf2 | 0.001048602 | 0.228178906 | 0.293 | 0.155 | 1 |
| Usp33 | 0.00105181 | 0.432922416 | 0.27 | 0.138 | 1 |
| Idh2 | 0.001053091 | 0.334148106 | 0.529 | 0.397 | 1 |
| Gpcpd1 | 0.001082717 | −0.21025995 | 0.253 | 0.408 | 1 |
| Slc37a4 | 0.001123966 | −0.359457571 | 0.276 | 0.414 | 1 |
| Hagh | 0.001130016 | −0.22804631 | 0.707 | 0.816 | 1 |
| Maob | 0.001142494 | −0.31742738 | 0.414 | 0.546 | 1 |
| Set | 0.001187474 | 0.254464638 | 0.374 | 0.23 | 1 |
| Ccne2 | 0.001209984 | 0.26317126 | 0.126 | 0.034 | 1 |
| Brk1 | 0.001215299 | 0.283487053 | 0.379 | 0.236 | 1 |
| Mrps16 | 0.001227646 | 0.211425725 | 0.287 | 0.149 | 1 |
| Pycrl | 0.001231949 | 0.223192893 | 0.31 | 0.172 | 1 |
| Fnbp1l | 0.001251014 | 0.220422078 | 0.236 | 0.109 | 1 |
| Ces1e | 0.00126714 | −0.364635829 | 0.385 | 0.511 | 1 |
| Ilf2 | 0.001274235 | 0.255415152 | 0.241 | 0.115 | 1 |
| Fam96a | 0.001315416 | 0.312612603 | 0.425 | 0.287 | 1 |
| Whsc1 | 0.001331048 | 0.318759797 | 0.356 | 0.218 | 1 |
| Dnajc10 | 0.001376642 | 0.246821061 | 0.368 | 0.218 | 1 |
| Lasp1 | 0.001377399 | 0.390083334 | 0.46 | 0.328 | 1 |
| Ddx39 | 0.001378811 | 0.2103436 | 0.224 | 0.103 | 1 |
| Pnpo | 0.00147794 | −0.309003494 | 0.471 | 0.603 | 1 |
| Rps3 | 0.001492324 | 0.212551746 | 0.851 | 0.816 | 1 |
| Sfmbt1 | 0.001558217 | 0.332239288 | 0.345 | 0.213 | 1 |
| Blvrb | 0.00157909 | 0.234388003 | 0.557 | 0.402 | 1 |
| Cebpa | 0.001599697 | −0.254983029 | 0.397 | 0.534 | 1 |
| mt-Nd5 | 0.001604259 | −0.276002669 | 0.816 | 0.891 | 1 |
| Glo1 | 0.001626667 | −0.292423358 | 0.517 | 0.621 | 1 |
| Cyp3a25 | 0.001638407 | −0.201190406 | 0.718 | 0.81 | 1 |
| Rpl38 | 0.001655339 | 0.231403686 | 0.218 | 0.103 | 1 |
| Haus3 | 0.001674455 | 0.209827476 | 0.172 | 0.069 | 1 |
| Hsd11b1 | 0.001710332 | −0.353381526 | 0.621 | 0.718 | 1 |
| Mrpl49 | 0.001820085 | 0.294845923 | 0.299 | 0.178 | 1 |
| Otud6b | 0.001827373 | 0.277520557 | 0.27 | 0.144 | 1 |
| Mup20 | 0.001879628 | −0.434017754 | 0.971 | 0.983 | 1 |
| Acsl1 | 0.001921669 | −0.27480221 | 0.649 | 0.764 | 1 |
| Nsmce4a | 0.001927311 | 0.223811104 | 0.322 | 0.19 | 1 |
| Tial1 | 0.001978044 | 0.253959367 | 0.351 | 0.207 | 1 |
| Rps7 | 0.001987208 | 0.202699839 | 0.207 | 0.092 | 1 |
| Polr2f | 0.002003606 | 0.232539505 | 0.385 | 0.247 | 1 |
| Snrpd2 | 0.002104173 | 0.246589047 | 0.351 | 0.213 | 1 |
| Clock | 0.002251358 | 0.294489034 | 0.328 | 0.201 | 1 |
| Pold3 | 0.002292673 | 0.222109813 | 0.195 | 0.086 | 1 |
| Snrpe | 0.002301841 | 0.224562608 | 0.305 | 0.172 | 1 |
| Mrpl51 | 0.002347961 | 0.215486219 | 0.345 | 0.213 | 1 |
| Gm4951 | 0.002357136 | −0.305065865 | 0.368 | 0.494 | 1 |
| Myo10 | 0.002366423 | 0.246835424 | 0.282 | 0.149 | 1 |
| Rcbtb2 | 0.002377524 | 0.238247244 | 0.282 | 0.155 | 1 |
| Pipox | 0.002416454 | −0.240575227 | 0.368 | 0.523 | 1 |
| Usp1 | 0.002481682 | 0.275810344 | 0.282 | 0.161 | 1 |
| Mup14 | 0.002489042 | −0.27120183 | 0.494 | 0.592 | 1 |
| Rpl37a | 0.002496318 | 0.256856261 | 0.339 | 0.207 | 1 |
| Acot1 | 0.002509249 | 0.201537266 | 0.253 | 0.132 | 1 |
| Suz12 | 0.002526887 | 0.280277593 | 0.31 | 0.178 | 1 |
| Srrt | 0.002534186 | 0.305896175 | 0.328 | 0.207 | 1 |
| Mup19 | 0.002541541 | −0.467484608 | 0.557 | 0.632 | 1 |
| Myo1c | 0.002559313 | 0.300308227 | 0.247 | 0.132 | 1 |
| Cds2 | 0.002587388 | 0.279313685 | 0.379 | 0.236 | 1 |
| Ncl | 0.002633004 | 0.316865625 | 0.655 | 0.575 | 1 |
| Stip1 | 0.002659945 | 0.28315643 | 0.483 | 0.379 | 1 |
| Hnrnpul1 | 0.002768498 | 0.244806598 | 0.391 | 0.264 | 1 |
| Adss | 0.00277119 | 0.30177346 | 0.385 | 0.259 | 1 |
| Rps19 | 0.002848401 | 0.249356214 | 0.575 | 0.448 | 1 |
| Eny2 | 0.002862696 | 0.261236803 | 0.5 | 0.362 | 1 |
| Krt8 | 0.002887003 | 0.274896508 | 0.621 | 0.489 | 1 |
| Mpv17l2 | 0.00292474 | 0.202123451 | 0.201 | 0.092 | 1 |
| Impad1 | 0.002949531 | 0.326238567 | 0.402 | 0.282 | 1 |
| Ugdh | 0.002968487 | 0.275976493 | 0.678 | 0.603 | 1 |
| Cth | 0.003053485 | −0.235854448 | 0.644 | 0.77 | 1 |
| Rps2 | 0.00308072 | 0.226119282 | 0.661 | 0.557 | 1 |
| Lman1 | 0.003235144 | −0.276383183 | 0.58 | 0.69 | 1 |
| Ebp | 0.003249538 | 0.276578351 | 0.695 | 0.592 | 1 |
| Timm9 | 0.003272703 | 0.246436845 | 0.328 | 0.201 | 1 |
| Caprin1 | 0.00327566 | 0.310159837 | 0.644 | 0.557 | 1 |
| Mup6 | 0.003278638 | −0.363001459 | 0.534 | 0.598 | 1 |
| Mmgt1 | 0.003323514 | 0.277568508 | 0.218 | 0.109 | 1 |
| Cdc42bpa | 0.003337449 | 0.296302291 | 0.27 | 0.149 | 1 |
| Gys2 | 0.003345786 | −0.266618519 | 0.247 | 0.362 | 1 |
| Tat | 0.003362872 | −0.204446885 | 0.891 | 0.948 | 1 |
| Arrdc3 | 0.003390694 | −0.274084677 | 0.201 | 0.328 | 1 |
| Decr2 | 0.003429858 | −0.248515355 | 0.471 | 0.58 | 1 |
| Fcna | 0.003546657 | 0.200314485 | 0.126 | 0.04 | 1 |
| Hspa4l | 0.003555845 | 0.224225179 | 0.356 | 0.224 | 1 |
| Faah | 0.003590172 | 0.223244291 | 0.511 | 0.362 | 1 |
| Dynll1 | 0.003631033 | 0.356189012 | 0.385 | 0.264 | 1 |
| Inmt | 0.003640717 | −0.256091272 | 0.684 | 0.793 | 1 |
| Smc1a | 0.003667723 | 0.26846514 | 0.362 | 0.236 | 1 |
| Mrps18a | 0.003669283 | 0.305636501 | 0.282 | 0.161 | 1 |
| Abhd6 | 0.003692342 | 0.230830064 | 0.368 | 0.236 | 1 |
| Cyp2c44 | 0.003724799 | −0.240647157 | 0.46 | 0.58 | 1 |
| Uroc1 | 0.003743767 | −0.284923771 | 0.517 | 0.609 | 1 |
| Marco | 0.003872675 | 0.460163442 | 0.149 | 0.057 | 1 |
| Uchl5 | 0.003891665 | 0.217812028 | 0.305 | 0.19 | 1 |
| Hamp | 0.003937766 | 0.278788719 | 0.718 | 0.609 | 1 |
| Pter | 0.003955936 | −0.298749309 | 0.264 | 0.385 | 1 |
| Insig1 | 0.004042461 | −0.246565903 | 0.443 | 0.563 | 1 |
| Cd302 | 0.004054903 | 0.216831932 | 0.879 | 0.868 | 1 |
| Ctsc | 0.004072786 | −0.224783491 | 0.19 | 0.316 | 1 |
| Med19 | 0.00426259 | 0.219441046 | 0.236 | 0.126 | 1 |
| Bckdha | 0.004281843 | −0.244274663 | 0.42 | 0.517 | 1 |
| Serinc3 | 0.004310832 | 0.234132553 | 0.494 | 0.379 | 1 |
| Saa4 | 0.004348051 | −0.261762833 | 0.753 | 0.81 | 1 |
| Gpr108 | 0.004425151 | −0.22603556 | 0.115 | 0.218 | 1 |
| Cmah | 0.004429229 | −0.212506919 | 0.385 | 0.523 | 1 |
| Mocs2 | 0.004501388 | −0.203503301 | 0.517 | 0.598 | 1 |
| Slc7a2 | 0.004632074 | −0.317210502 | 0.598 | 0.713 | 1 |
| Strap | 0.004743896 | 0.228584031 | 0.466 | 0.339 | 1 |
| Clec4f | 0.004797027 | 0.23190205 | 0.218 | 0.109 | 1 |
| Rpl22 | 0.004937805 | 0.275204914 | 0.546 | 0.42 | 1 |
| Elp5 | 0.004998547 | 0.209886016 | 0.218 | 0.109 | 1 |
| Serpina3m | 0.005024551 | 0.243811964 | 0.77 | 0.77 | 1 |
| Ywhag | 0.005074343 | 0.288452561 | 0.517 | 0.414 | 1 |
| Arpc1b | 0.00508026 | 0.245172175 | 0.305 | 0.178 | 1 |
| Nptn | 0.005127487 | 0.287320757 | 0.339 | 0.213 | 1 |
| Ces1b | 0.005217312 | 0.200087271 | 0.494 | 0.374 | 1 |
| Pdxk | 0.005251936 | 0.243284689 | 0.23 | 0.121 | 1 |
| Dpy30 | 0.005500491 | 0.216655337 | 0.316 | 0.19 | 1 |
| 1810037I17Rik | 0.005778623 | 0.211310694 | 0.345 | 0.218 | 1 |
| Rbbp4 | 0.005932398 | 0.310895251 | 0.477 | 0.356 | 1 |
| Snord13 | 0.006012681 | 0.417208326 | 0.46 | 0.333 | 1 |
| Csf1r | 0.006160528 | 0.260730004 | 0.132 | 0.046 | 1 |
| Nicn1 | 0.006222528 | 0.226145504 | 0.236 | 0.126 | 1 |
| Mup12 | 0.006246858 | −0.441572158 | 0.489 | 0.569 | 1 |
| Coq2 | 0.006338538 | 0.228017679 | 0.328 | 0.213 | 1 |
| Yme1l1 | 0.006361678 | 0.223955561 | 0.575 | 0.448 | 1 |
| Opa3 | 0.006456608 | 0.275149939 | 0.345 | 0.224 | 1 |
| Atp2c1 | 0.006528816 | 0.218202897 | 0.259 | 0.144 | 1 |
| Sun2 | 0.006773287 | 0.244395261 | 0.368 | 0.247 | 1 |
| Sar1b | 0.006796779 | −0.238677843 | 0.5 | 0.621 | 1 |
| Cenpb | 0.007197198 | 0.29209786 | 0.379 | 0.27 | 1 |
| Ftcd | 0.007254799 | −0.236915896 | 0.517 | 0.603 | 1 |
| Calu | 0.007575412 | 0.204995826 | 0.391 | 0.264 | 1 |
| Il1rap | 0.007778177 | −0.302722178 | 0.374 | 0.477 | 1 |
| Psmc5 | 0.008043174 | 0.213987203 | 0.557 | 0.414 | 1 |
| Smarcc1 | 0.0080442 | 0.207630543 | 0.345 | 0.23 | 1 |
| Actb | 0.008170796 | 0.20065238 | 0.902 | 0.96 | 1 |
| Rnf141 | 0.008612042 | 0.243430584 | 0.374 | 0.259 | 1 |
| Hint1 | 0.008673406 | 0.278109318 | 0.753 | 0.73 | 1 |
| Plxna2 | 0.00875936 | −0.211045239 | 0.052 | 0.132 | 1 |
| Csad | 0.008778135 | 0.237189009 | 0.557 | 0.431 | 1 |
| Gtf3c4 | 0.008803983 | 0.200525731 | 0.218 | 0.115 | 1 |
| Apex1 | 0.008816562 | 0.247646366 | 0.27 | 0.161 | 1 |
| Slc40a1 | 0.008850796 | 0.28276667 | 0.391 | 0.259 | 1 |
| B230219D22Rik | 0.008870372 | 0.21573072 | 0.345 | 0.218 | 1 |
| Rps5 | 0.008922041 | 0.308634542 | 0.839 | 0.845 | 1 |
| Mup4 | 0.008927704 | −0.278610154 | 0.305 | 0.402 | 1 |
| Aqp8 | 0.008944689 | −0.248202502 | 0.259 | 0.374 | 1 |
| Sh3bgrl | 0.008960366 | 0.304384057 | 0.557 | 0.414 | 1 |
| Cxcl1 | 0.009048319 | −0.30974824 | 0.368 | 0.489 | 1 |
| Acad11 | 0.0091016 | −0.279085199 | 0.397 | 0.5 | 1 |
| Os9 | 0.009262437 | −0.241560677 | 0.523 | 0.632 | 1 |
| Cct2 | 0.009296317 | 0.26056313 | 0.609 | 0.506 | 1 |
| Slc17a2 | 0.009411837 | −0.225152117 | 0.299 | 0.408 | 1 |
| Pcyt2 | 0.009491776 | −0.265725588 | 0.46 | 0.603 | 1 |
| Cacybp | 0.009506321 | 0.208121446 | 0.356 | 0.236 | 1 |
| H3f3b | 0.009520983 | 0.242133513 | 0.54 | 0.454 | 1 |
| Cpped1 | 0.009650292 | 0.245780009 | 0.425 | 0.31 | 1 |
| Arpc4 | 0.009701026 | 0.244147674 | 0.299 | 0.195 | 1 |
| mt-Nd4 | 0.009806959 | −0.330562447 | 0.851 | 0.868 | 1 |
| Cyp2d9 | 0.009863302 | −0.21251377 | 0.764 | 0.885 | 1 |
| Brd7 | 0.01022504 | 0.24880251 | 0.31 | 0.201 | 1 |
| Lmo4 | 0.01042178 | 0.230146494 | 0.224 | 0.121 | 1 |
| Mrpl18 | 0.010449979 | 0.271017845 | 0.385 | 0.27 | 1 |
| Mnrnpl | 0.010478934 | 0.20130563 | 0.333 | 0.224 | 1 |
| Ndufb10 | 0.010581051 | −0.230971243 | 0.557 | 0.644 | 1 |
| Hdgf | 0.011006886 | 0.298205008 | 0.552 | 0.477 | 1 |
| Ndufb7 | 0.011093102 | 0.281504386 | 0.569 | 0.454 | 1 |
| Kras | 0.011161828 | 0.239901841 | 0.328 | 0.218 | 1 |
| Mrpl33 | 0.011165541 | 0.295337683 | 0.443 | 0.345 | 1 |
| Gstm2 | 0.011172361 | 0.209182349 | 0.385 | 0.259 | 1 |
| Dap3 | 0.011395912 | 0.310029486 | 0.345 | 0.241 | 1 |
| Atl2 | 0.011399898 | 0.28646695 | 0.379 | 0.264 | 1 |
| Rpl26 | 0.011418585 | 0.302442081 | 0.632 | 0.523 | 1 |
| Abcb6 | 0.011429815 | 0.280241248 | 0.385 | 0.27 | 1 |
| Lipa | 0.011468112 | 0.200015494 | 0.684 | 0.603 | 1 |
| Banf1 | 0.01186588 | 0.236631164 | 0.356 | 0.253 | 1 |
| Orm2 | 0.011955778 | 0.216695694 | 0.695 | 0.575 | 1 |
| Tmsb4x | 0.012156792 | 0.352694664 | 0.241 | 0.138 | 1 |
| Leap2 | 0.012207481 | −0.300816683 | 0.408 | 0.517 | 1 |
| Casp8 | 0.012221596 | 0.213097538 | 0.236 | 0.138 | 1 |
| Srsf1 | 0.012357324 | 0.274604273 | 0.356 | 0.247 | 1 |
| Khk | 0.012440505 | −0.249137231 | 0.678 | 0.741 | 1 |
| Carhsp1 | 0.012531079 | 0.319710888 | 0.379 | 0.299 | 1 |
| Slc25a10 | 0.012542423 | 0.207832074 | 0.529 | 0.408 | 1 |
| Dis3l | 0.01263226 | 0.218699045 | 0.213 | 0.121 | 1 |
| Vps36 | 0.012987505 | 0.274038809 | 0.339 | 0.236 | 1 |
| Qdpr | 0.013483198 | −0.211506384 | 0.713 | 0.747 | 1 |
| Dnm1l | 0.013510207 | 0.234951606 | 0.362 | 0.259 | 1 |
| Hpgd | 0.013665829 | 0.226866333 | 0.425 | 0.322 | 1 |
| Triap1 | 0.013715135 | 0.206958913 | 0.207 | 0.115 | 1 |
| Ddah1 | 0.013913907 | −0.229307183 | 0.322 | 0.443 | 1 |
| Rhod | 0.014224771 | 0.203809463 | 0.282 | 0.184 | 1 |
| Mcm10 | 0.014232039 | −0.232484957 | 0.224 | 0.322 | 1 |
| Ibtk | 0.014339165 | 0.230852193 | 0.391 | 0.276 | 1 |
| Immt | 0.014527035 | 0.314198334 | 0.517 | 0.414 | 1 |
| Slc22a30 | 0.014677509 | −0.251268208 | 0.328 | 0.425 | 1 |
| Nudcd2 | 0.014812869 | 0.248670133 | 0.328 | 0.23 | 1 |
| Nsmce1 | 0.01486159 | 0.252864026 | 0.322 | 0.224 | 1 |
| Cpne3 | 0.015250747 | 0.294236673 | 0.511 | 0.402 | 1 |
| Slc3a2 | 0.015808689 | 0.219634219 | 0.236 | 0.144 | 1 |
| Fam210b | 0.01604669 | −0.22593416 | 0.397 | 0.5 | 1 |
| Hspd1 | 0.016067675 | 0.23534371 | 0.615 | 0.557 | 1 |
| Adipor2 | 0.016193716 | −0.257034087 | 0.718 | 0.776 | 1 |
| Oat | 0.016305262 | 0.292703373 | 0.5 | 0.397 | 1 |
| Gnai2 | 0.016596229 | 0.256616592 | 0.466 | 0.339 | 1 |
| Mob3b | 0.016709013 | 0.211167706 | 0.236 | 0.144 | 1 |
| Sumo3 | 0.016883831 | 0.295186967 | 0.471 | 0.391 | 1 |
| 2700060E02Rik | 0.016991549 | 0.246273177 | 0.408 | 0.305 | 1 |
| Pbld1 | 0.016999839 | −0.200773785 | 0.339 | 0.431 | 1 |
| Ctcf | 0.017060197 | 0.29778649 | 0.293 | 0.195 | 1 |
| Tmx2 | 0.017182975 | 0.208652952 | 0.322 | 0.224 | 1 |
| Eif6 | 0.017376562 | 0.220475649 | 0.408 | 0.31 | 1 |
| Saa1 | 0.017409969 | 0.235927452 | 0.937 | 0.879 | 1 |
| Gm6484 | 0.017707724 | −0.239084424 | 0.103 | 0.184 | 1 |
| Ppp2r5e | 0.017731156 | 0.213917029 | 0.362 | 0.264 | 1 |
| Rps27l | 0.017839688 | 0.259957822 | 0.701 | 0.672 | 1 |
| Shoc2 | 0.0179067 | 0.227313307 | 0.339 | 0.23 | 1 |
| Rdh11 | 0.017989816 | 0.234866085 | 0.443 | 0.339 | 1 |
| Fbxo30 | 0.018061788 | 0.247683896 | 0.167 | 0.086 | 1 |
| Pds5a | 0.018213315 | 0.2096822 | 0.264 | 0.167 | 1 |
| Nfyb | 0.018514036 | 0.201736814 | 0.27 | 0.172 | 1 |
| Rpl36al | 0.01867848 | 0.227430291 | 0.511 | 0.414 | 1 |
| Dpyd | 0.018939626 | −0.207660551 | 0.494 | 0.598 | 1 |
| Keap1 | 0.019209184 | 0.224305051 | 0.402 | 0.293 | 1 |
| Gzf1 | 0.019213526 | 0.294348759 | 0.339 | 0.236 | 1 |
| Gfm1 | 0.019244456 | 0.206633144 | 0.425 | 0.322 | 1 |
| Mbd3 | 0.019279057 | 0.262066315 | 0.379 | 0.282 | 1 |
| Gapvd1 | 0.019311281 | 0.223374216 | 0.293 | 0.195 | 1 |
| Txn2 | 0.019513804 | −0.205404835 | 0.46 | 0.557 | 1 |
| Sgk2 | 0.019702868 | −0.281901472 | 0.195 | 0.287 | 1 |
| Mrpl48 | 0.019837016 | 0.203084366 | 0.224 | 0.132 | 1 |
| Hebp1 | 0.020095167 | −0.21588023 | 0.546 | 0.644 | 1 |
| Arpp19 | 0.020184043 | 0.248525375 | 0.471 | 0.368 | 1 |
| G3bp2 | 0.020214404 | 0.254441829 | 0.477 | 0.374 | 1 |
| D17Wsu92e | 0.020375994 | 0.258509249 | 0.598 | 0.563 | 1 |
| Prkd3 | 0.020437553 | −0.230881843 | 0.431 | 0.529 | 1 |
| Lcn2 | 0.020792109 | 0.206498859 | 0.667 | 0.534 | 1 |
| Wapal | 0.020803332 | 0.23807797 | 0.517 | 0.42 | 1 |
| Cct5 | 0.020920087 | 0.285043345 | 0.603 | 0.534 | 1 |
| Retsat | 0.021162383 | −0.327711821 | 0.511 | 0.557 | 1 |
| Saa2 | 0.021583651 | 0.251309011 | 0.77 | 0.724 | 1 |
| Szrd1 | 0.021627753 | 0.220282685 | 0.385 | 0.282 | 1 |
| Etnk2 | 0.021720372 | −0.243330541 | 0.546 | 0.609 | 1 |
| Eef1d | 0.02187914 | 0.247282181 | 0.437 | 0.339 | 1 |
| Nup88 | 0.022150869 | 0.24232933 | 0.218 | 0.132 | 1 |
| Psmd2 | 0.022305574 | 0.243358975 | 0.511 | 0.443 | 1 |
| Fkbp2 | 0.022625189 | 0.250554432 | 0.546 | 0.437 | 1 |
| Dnajc22 | 0.022643266 | 0.26166465 | 0.552 | 0.494 | 1 |
| Blvra | 0.022971107 | 0.242991088 | 0.253 | 0.161 | 1 |
| Hsd3b3 | 0.023233447 | −0.258431285 | 0.425 | 0.517 | 1 |
| Dpp4 | 0.023405669 | 0.203659148 | 0.454 | 0.356 | 1 |
| Tmem106b | 0.023617735 | −0.230722925 | 0.448 | 0.523 | 1 |
| Akr1c19 | 0.023755169 | 0.23338723 | 0.466 | 0.362 | 1 |
| Egr1 | 0.023819407 | 0.220517026 | 0.494 | 0.414 | 1 |
| Sppl2a | 0.023896904 | 0.225826218 | 0.615 | 0.506 | 1 |
| Map1lc3b | 0.023897255 | −0.207314779 | 0.264 | 0.356 | 1 |
| Wdr48 | 0.024293713 | 0.21960522 | 0.27 | 0.184 | 1 |
| Dpys | 0.024446167 | −0.256803998 | 0.466 | 0.534 | 1 |
| Eif3h | 0.024456091 | 0.294440454 | 0.575 | 0.506 | 1 |
| Rer1 | 0.024496578 | 0.226471902 | 0.431 | 0.339 | 1 |
| Zfp91 | 0.024540292 | 0.251453484 | 0.546 | 0.466 | 1 |
| Utp3 | 0.024843557 | 0.265694599 | 0.362 | 0.259 | 1 |
| Dhx15 | 0.024933924 | 0.300789082 | 0.368 | 0.276 | 1 |
| Syne2 | 0.025020753 | 0.221539341 | 0.287 | 0.19 | 1 |
| Txndc9 | 0.025204473 | 0.320248454 | 0.287 | 0.195 | 1 |
| H2afy | 0.025209744 | 0.208626457 | 0.529 | 0.437 | 1 |
| Abat | 0.025345228 | −0.240152398 | 0.58 | 0.649 | 1 |
| Ndufa8 | 0.025803507 | 0.205315445 | 0.615 | 0.517 | 1 |
| Atg12 | 0.026361167 | 0.216199712 | 0.31 | 0.213 | 1 |
| Mnrnpm | 0.026509991 | 0.237984435 | 0.362 | 0.282 | 1 |
| Ipo5 | 0.026527598 | 0.209133978 | 0.276 | 0.19 | 1 |
| Mycbp | 0.026954934 | 0.203356679 | 0.264 | 0.172 | 1 |
| Mt2 | 0.026971886 | 0.31805591 | 0.695 | 0.609 | 1 |
| Dnajc2 | 0.027055074 | 0.203873598 | 0.236 | 0.149 | 1 |
| Cwf19l2 | 0.02729564 | 0.250617416 | 0.201 | 0.121 | 1 |
| Hibch | 0.027978397 | −0.286834561 | 0.276 | 0.374 | 1 |
| Dsp | 0.028292845 | −0.24308704 | 0.19 | 0.282 | 1 |
| Exoc4 | 0.028722276 | 0.270709894 | 0.293 | 0.201 | 1 |
| Sorbs2 | 0.028909173 | 0.213041815 | 0.397 | 0.299 | 1 |
| Rps18 | 0.028943546 | 0.202877972 | 0.632 | 0.529 | 1 |
| Fxr2 | 0.029093243 | 0.268694616 | 0.213 | 0.126 | 1 |
| Hras | 0.030100817 | 0.299616126 | 0.431 | 0.356 | 1 |
| Tcf4 | 0.030183418 | 0.201428644 | 0.149 | 0.08 | 1 |
| Rpl3 | 0.030330012 | 0.292268532 | 0.391 | 0.293 | 1 |
| Mrpl54 | 0.030987433 | 0.211022994 | 0.339 | 0.241 | 1 |
| Copz1 | 0.031526118 | −0.286190248 | 0.374 | 0.46 | 1 |
| Lrpprc | 0.033102616 | −0.236064383 | 0.385 | 0.477 | 1 |
| Ndufs6 | 0.03506636 | 0.215815094 | 0.598 | 0.529 | 1 |
| Aim1 | 0.035806526 | 0.219520531 | 0.287 | 0.195 | 1 |
| Saa3 | 0.036302206 | 0.577607147 | 0.391 | 0.299 | 1 |
| Lrrc58 | 0.036390449 | −0.240695487 | 0.333 | 0.431 | 1 |
| Decr1 | 0.036405359 | −0.215322531 | 0.517 | 0.615 | 1 |
| Spag9 | 0.036695211 | −0.212359016 | 0.282 | 0.379 | 1 |
| Tmed7 | 0.037888709 | −0.205329015 | 0.414 | 0.483 | 1 |
| Entpd5 | 0.038227053 | 0.210104663 | 0.621 | 0.557 | 1 |
| Tcerg1 | 0.038897924 | 0.267832269 | 0.195 | 0.121 | 1 |
| Acsf2 | 0.038897924 | −0.219388645 | 0.121 | 0.195 | 1 |
| Sptssa | 0.038938052 | 0.222943638 | 0.443 | 0.351 | 1 |
| Ap3b1 | 0.039319143 | 0.301743969 | 0.362 | 0.276 | 1 |
| Pbf14 | 0.040853389 | 0.246441091 | 0.236 | 0.149 | 1 |
| Mfn2 | 0.04092404 | 0.2025045 | 0.391 | 0.305 | 1 |
| Gfer | 0.042628023 | 0.240942535 | 0.339 | 0.259 | 1 |
| Nfia | 0.043029792 | 0.211281834 | 0.374 | 0.282 | 1 |
| Trappc13 | 0.043101637 | 0.217746706 | 0.259 | 0.178 | 1 |
| Tbl1x | 0.043601109 | 0.204898253 | 0.402 | 0.31 | 1 |
| Msl3 | 0.045102788 | 0.236211559 | 0.247 | 0.167 | 1 |
| Efna1 | 0.045838317 | −0.205634717 | 0.305 | 0.379 | 1 |
| Nipsnap1 | 0.046051269 | −0.220923712 | 0.615 | 0.661 | 1 |
| Magt1 | 0.046203205 | −0.211713805 | 0.402 | 0.466 | 1 |
| Ddt | 0.046305466 | 0.206340801 | 0.672 | 0.615 | 1 |
| Ndufa11 | 0.046330307 | 0.229735966 | 0.443 | 0.362 | 1 |
| C1qb | 0.046414021 | 0.391357306 | 0.167 | 0.098 | 1 |
| Myh10 | 0.046442872 | 0.243905291 | 0.218 | 0.144 | 1 |
| Rpl39 | 0.046825507 | 0.220871962 | 0.437 | 0.362 | 1 |
| Cdc34 | 0.046827233 | 0.244606779 | 0.534 | 0.471 | 1 |
| Tstd2 | 0.047364738 | 0.222703062 | 0.195 | 0.121 | 1 |
| Aldh6a1 | 0.047461614 | −0.235184768 | 0.54 | 0.603 | 1 |
| Anp32a | 0.047479235 | 0.247594131 | 0.408 | 0.333 | 1 |
| Serpinf1 | 0.047490175 | −0.20019453 | 0.713 | 0.73 | 1 |
| Gm24601 | 0.048145145 | 0.231716349 | 0.391 | 0.293 | 1 |
| BC005537 | 0.048785835 | 0.298901807 | 0.5 | 0.42 | 1 |
| Sfxn1 | 0.049152777 | −0.240207413 | 0.402 | 0.471 | 1 |
| Fubp1 | 0.049710915 | 0.203229491 | 0.241 | 0.167 | 1 |
| Mnrnpc | 0.050896017 | 0.200044122 | 0.448 | 0.351 | 1 |
| Grip2 | 0.051111051 | −0.619607732 | 0.149 | 0.236 | 1 |
| Cnot1 | 0.051175552 | 0.218985315 | 0.31 | 0.23 | 1 |
| Ggnbp2 | 0.051982034 | 0.21529549 | 0.471 | 0.368 | 1 |
| Gss | 0.052057686 | 0.22604635 | 0.454 | 0.374 | 1 |
| Aspg | 0.052401512 | −0.222298996 | 0.224 | 0.287 | 1 |
| Chd8 | 0.052401541 | 0.242974398 | 0.299 | 0.213 | 1 |
| Uqcr10 | 0.053864701 | 0.264431516 | 0.718 | 0.644 | 1 |
| Lyz2 | 0.054277151 | 0.29805528 | 0.167 | 0.098 | 1 |
| Trpc4ap | 0.056052262 | 0.2138402 | 0.293 | 0.218 | 1 |
| Specc1l | 0.056478111 | 0.218824052 | 0.161 | 0.098 | 1 |
| Cd163 | 0.057321755 | 0.269431124 | 0.098 | 0.046 | 1 |
| Cldn2 | 0.057579448 | −0.215656114 | 0.236 | 0.305 | 1 |
| Cd2ap | 0.05768152 | 0.23579619 | 0.276 | 0.201 | 1 |
| Qser1 | 0.057690417 | 0.283756443 | 0.213 | 0.144 | 1 |
| Mup1 | 0.058856335 | −0.208101355 | 0.397 | 0.46 | 1 |
| Cisd2 | 0.060516248 | 0.208851947 | 0.42 | 0.333 | 1 |
| Casp3 | 0.062306591 | 0.210912344 | 0.259 | 0.19 | 1 |
| Stub1 | 0.062888331 | 0.224769038 | 0.414 | 0.356 | 1 |
| Adrbk1 | 0.063207706 | 0.206901175 | 0.155 | 0.092 | 1 |
| Gm10800 | 0.065789182 | −0.295424977 | 0.023 | 0.063 | 1 |
| Vapa | 0.065815815 | 0.211922971 | 0.397 | 0.305 | 1 |
| Ndufaf2 | 0.066094467 | 0.208054391 | 0.339 | 0.264 | 1 |
| Gch1 | 0.06613008 | 0.207064291 | 0.557 | 0.511 | 1 |
| Mxd4 | 0.066182986 | −0.224203201 | 0.224 | 0.293 | 1 |
| Tor1b | 0.066283436 | 0.214789628 | 0.27 | 0.201 | 1 |
| Deptor | 0.068470633 | −0.219528623 | 0.397 | 0.46 | 1 |
| Eif3d | 0.068969412 | 0.212677163 | 0.42 | 0.351 | 1 |
| Cfl2 | 0.069774684 | 0.204527974 | 0.414 | 0.351 | 1 |
| Dnm2 | 0.069841224 | 0.201366691 | 0.287 | 0.213 | 1 |
| 2810004N23Rik | 0.071267147 | 0.279868496 | 0.351 | 0.27 | 1 |
| Mrpl20 | 0.073361269 | 0.207221171 | 0.466 | 0.391 | 1 |
| Ildr2 | 0.074426769 | 0.34186496 | 0.333 | 0.27 | 1 |
| Gars | 0.077067731 | 0.213365361 | 0.293 | 0.224 | 1 |
| Ctnnd1 | 0.077946124 | 0.231250746 | 0.339 | 0.259 | 1 |
| A1cf | 0.078522619 | 0.214908799 | 0.5 | 0.443 | 1 |
| Nmt2 | 0.082282004 | 0.207030081 | 0.218 | 0.155 | 1 |
| Rps24 | 0.083491925 | 0.240979347 | 0.632 | 0.569 | 1 |
| Ndufa3 | 0.085532305 | −0.205539056 | 0.483 | 0.552 | 1 |
| Ces1g | 0.086135024 | 0.228893144 | 0.517 | 0.477 | 1 |
| Cops3 | 0.088068119 | 0.222657556 | 0.362 | 0.287 | 1 |
| Arid4b | 0.088454061 | 0.22799984 | 0.236 | 0.172 | 1 |
| Litaf | 0.093099022 | 0.222901848 | 0.425 | 0.339 | 1 |
| Polr3c | 0.093769073 | 0.216488984 | 0.23 | 0.161 | 1 |
| Chordc1 | 0.094283632 | 0.24072197 | 0.374 | 0.287 | 1 |
| Anapc13 | 0.094582243 | 0.201825147 | 0.425 | 0.356 | 1 |
| Rpph1 | 0.098448086 | 0.202132446 | 0.23 | 0.167 | 1 |
| Mup9 | 0.099745235 | −0.271781283 | 0.368 | 0.443 | 1 |
| Eif5b | 0.10119655 | 0.218495171 | 0.443 | 0.362 | 1 |
| Trpm7 | 0.101862919 | 0.224519601 | 0.345 | 0.27 | 1 |
| Gsto1 | 0.102193646 | −0.242358799 | 0.322 | 0.374 | 1 |
| Hbb-bs | 0.103010489 | −0.208517986 | 0.075 | 0.126 | 1 |
| Zfp191 | 0.103122269 | 0.223545853 | 0.282 | 0.213 | 1 |
| Rock1 | 0.104733377 | 0.251852561 | 0.362 | 0.31 | 1 |
| Trmt2a | 0.10631232 | 0.201837146 | 0.155 | 0.098 | 1 |
| Hmgn5 | 0.108114199 | 0.200609931 | 0.379 | 0.316 | 1 |
| Mrpl13 | 0.110202369 | 0.238057228 | 0.5 | 0.454 | 1 |
| Brcc3 | 0.111565681 | 0.20572452 | 0.276 | 0.218 | 1 |
| Tspan9 | 0.112025748 | 0.211703014 | 0.115 | 0.069 | 1 |
| Snrnp200 | 0.115532314 | 0.217488694 | 0.264 | 0.195 | 1 |
| Fech | 0.1178627 | 0.212953066 | 0.557 | 0.489 | 1 |
| Slc4a4 | 0.11806357 | 0.20735045 | 0.414 | 0.339 | 1 |
| Eif3g | 0.119048105 | 0.200216768 | 0.385 | 0.322 | 1 |
| Gpr146 | 0.120071427 | −0.251942829 | 0.293 | 0.351 | 1 |
| Cnih1 | 0.125250208 | 0.219317444 | 0.368 | 0.316 | 1 |
| Actr3 | 0.12982029 | 0.20860417 | 0.287 | 0.236 | 1 |
| Ppm1k | 0.129962879 | 0.324630558 | 0.511 | 0.483 | 1 |
| Ablim3 | 0.135177431 | −0.223008512 | 0.086 | 0.132 | 1 |
| Cast | 0.139729608 | 0.261775559 | 0.339 | 0.276 | 1 |
| Papola | 0.142115112 | 0.225706284 | 0.471 | 0.425 | 1 |
| Slco1b2 | 0.143447728 | −0.212121676 | 0.874 | 0.908 | 1 |
| Pctp | 0.150651256 | −0.207440425 | 0.293 | 0.345 | 1 |
| Rps15a | 0.153126618 | 0.245141756 | 0.351 | 0.293 | 1 |
| Nckap1 | 0.160335985 | 0.210159632 | 0.397 | 0.345 | 1 |
| Plekha8 | 0.165399995 | 0.226910035 | 0.201 | 0.149 | 1 |
| Pbrm1 | 0.165692031 | 0.200710098 | 0.351 | 0.293 | 1 |
| Pgm3 | 0.177901845 | 0.20414768 | 0.241 | 0.184 | 1 |
| D8Ertd738e | 0.203182332 | 0.241338629 | 0.466 | 0.425 | 1 |
| Fyttd1 | 0.241823018 | 0.207298863 | 0.305 | 0.247 | 1 |
| Tbl1xr1 | 0.260541136 | 0.233716463 | 0.299 | 0.253 | 1 |
| Btg2 | 0.269475652 | 0.213428173 | 0.362 | 0.328 | 1 |
| Apoa4 | 0.29179639 | 0.238743063 | 0.563 | 0.546 | 1 |
| Actn4 | 0.315895266 | 0.223722393 | 0.489 | 0.46 | 1 |
| Gcc2 | 0.316131341 | 0.217374612 | 0.236 | 0.195 | 1 |
| Larp4b | 0.338255167 | 0.205260189 | 0.368 | 0.345 | 1 |
| Ptcd2 | 0.391938515 | 0.247969764 | 0.207 | 0.172 | 1 |
| n-R5-8s1 | 0.400728491 | 0.233544882 | 0.523 | 0.506 | 1 |
| Pcyt1a | 0.423598847 | 0.275819635 | 0.374 | 0.339 | 1 |
| Mettl16 | 0.458903759 | 0.204651364 | 0.201 | 0.172 | 1 |
| Mgea5 | 0.499176875 | 0.213978639 | 0.339 | 0.322 | 1 |
| Gm26917 | 0.514428432 | −0.298541162 | 0.293 | 0.328 | 1 |
| Marf1 | 0.515620318 | 0.236482432 | 0.213 | 0.195 | 1 |
| Wdr3 | 0.682566958 | 0.204292215 | 0.155 | 0.144 | 1 |
| Map3k12 | 0.755668288 | −0.225875211 | 0.029 | 0.034 | 1 |
| Cyp2a5 | 0.771556335 | 0.324941979 | 0.316 | 0.328 | 1 |
| Pdpk1 | 0.934116563 | −0.229596112 | 0.213 | 0.213 | 1 |

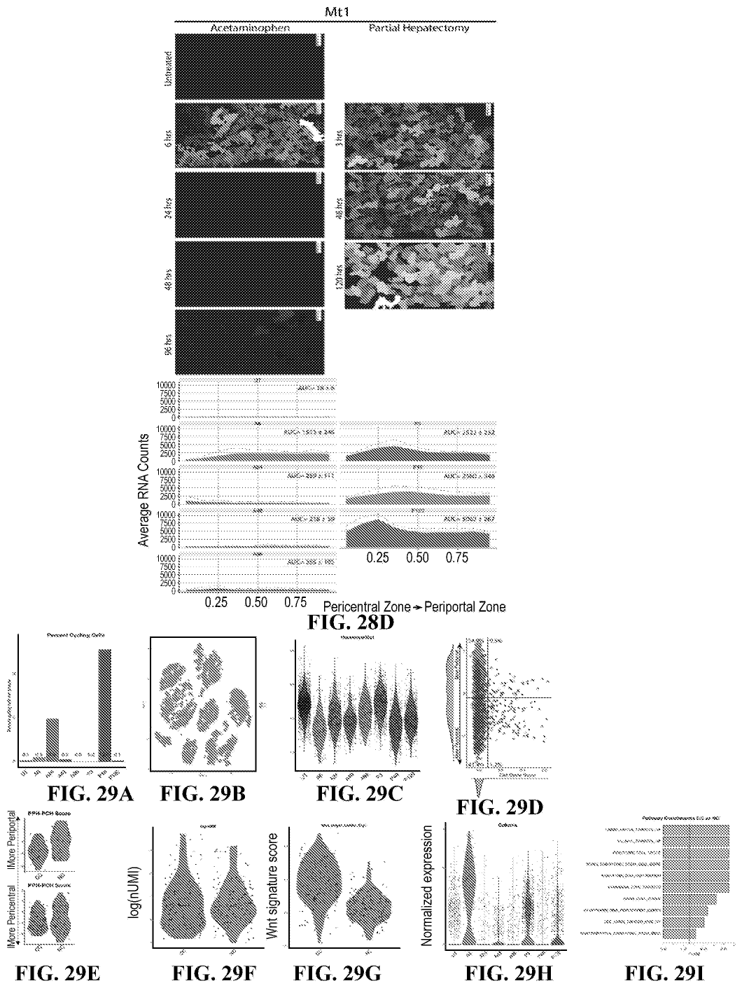

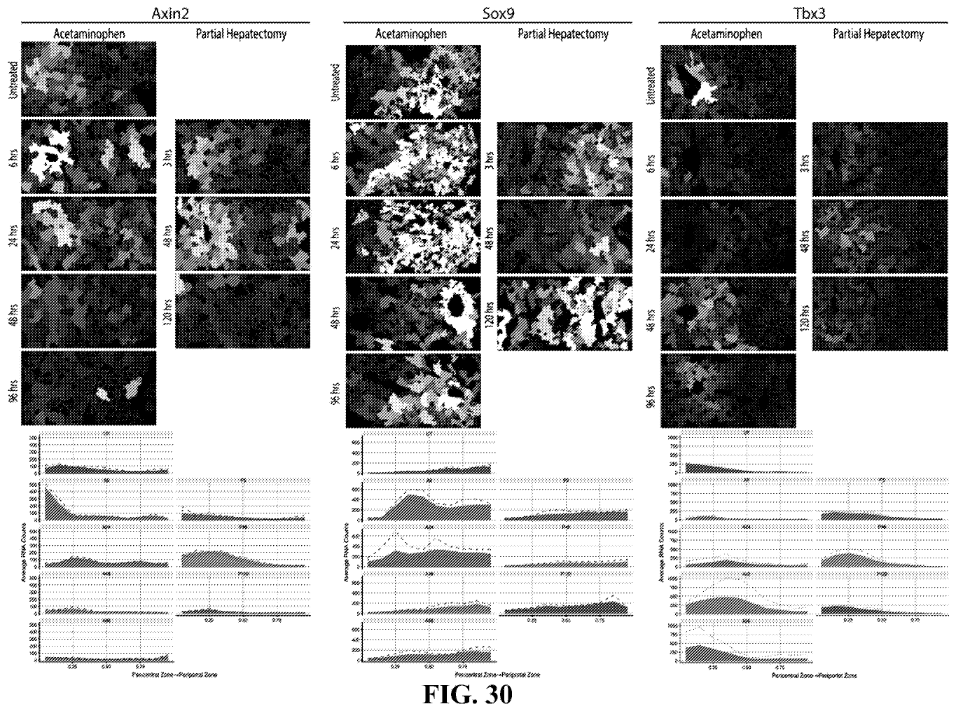

To identify pathways and potential upstream regulators involved in cell cycle activation, Applicants performed GSA over DEG calculated between CCs and NCs from A24 and P48, revealing an upregulation of cell cycle-related pathways. Further, there was a strong enrichment for Wnt-related pathways (FIG. 29). It has been shown extensively that Wnt signaling is involved with normal hepatocyte turnover and liver regeneration^{10,12,21-24,13-20} These Wnt factors are thought to be derived from the endothelium and contribute to the activation of hepatic progenitor cell genes (Axin2, Tbx3, and Sox9)^{12,30}. Applicants observed up-regulation of Axin2, Tbx3, and Sox9 in each acute injury model, with expression reaching multiple cell layers into the midzone FIG. 30). These data support activation of WNT/-catenin signaling in response to injury, which may be co-localized with the induction of genes associated with hepatic plasticity^{12,30}. This could explain the observation that hepatocytes outside of a particular zone have the ability to activate the expression of genes that may be lost due to zone-specific injury (APAP) or extreme tissue loss (PH), such as midzonal hepatocytes up-regulating Glu1 in either injury model.

### Wnt Signaling Mediates Functional Compensation in Addition to Proliferative Response

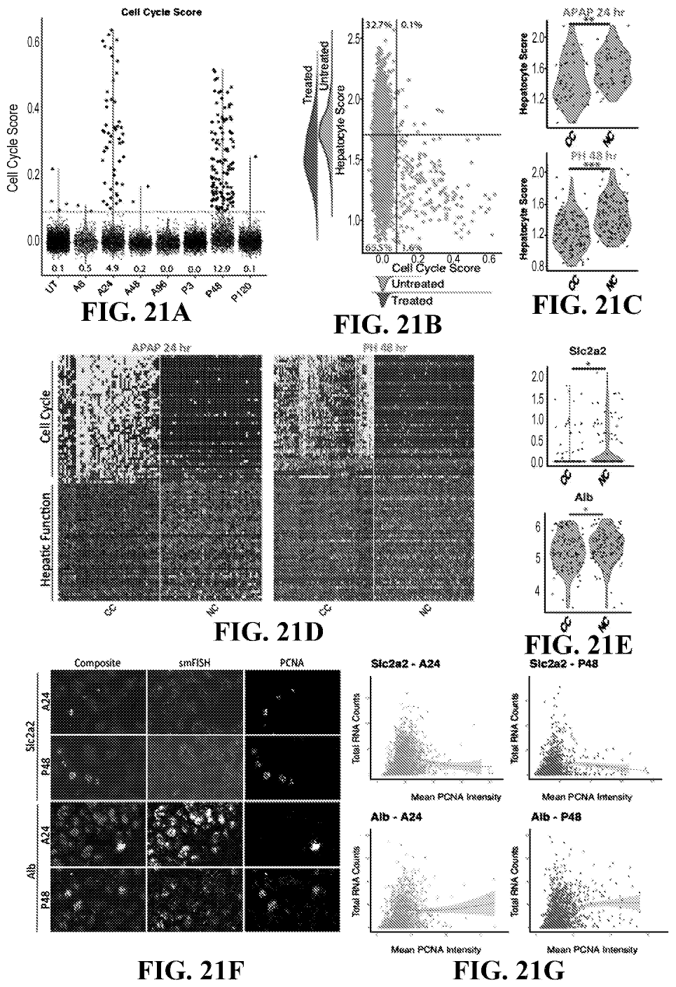

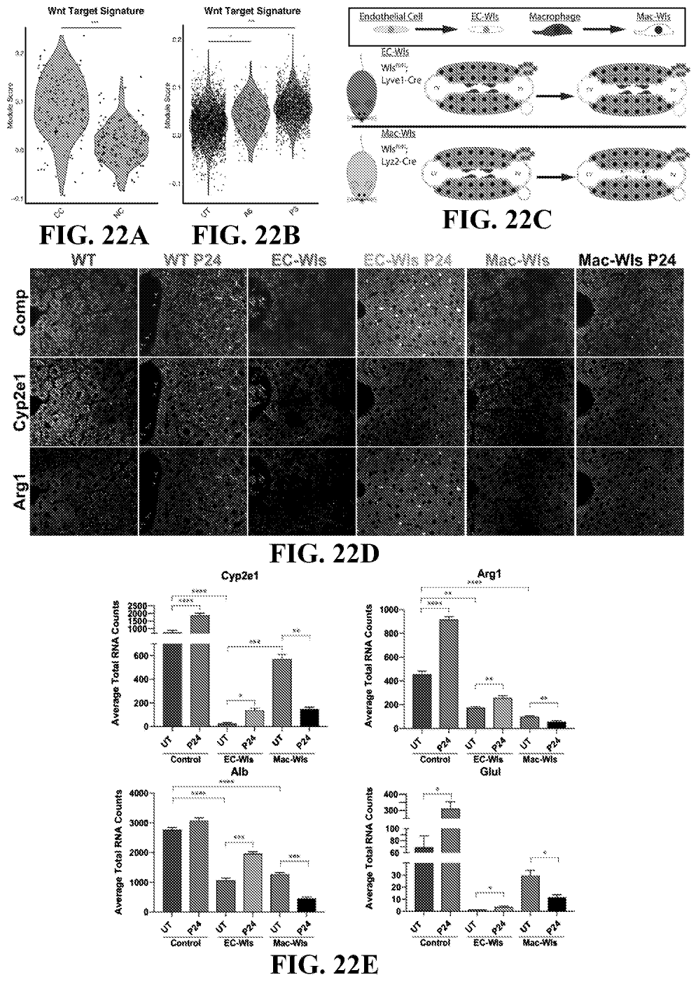

Given the demonstrated role of Wnt signaling in establishing liver zonation and proliferation during liver regeneration, Applicants investigated whether Wnt signaling might activate adaptation of already present hepatocytes to maintain essential hepatic function^{13,31-34}. scRNA-seq data corroborated previous observations of increased Wnt activity in proliferating hepatocytes (FIG. 22A)^{17,34,35}. Further, it revealed activation of Wnt target gene expression in the majority of hepatocytes in both the APAP and PH models, preceding the onset of cell proliferative activity (A6 and P3, FIG. 22B).

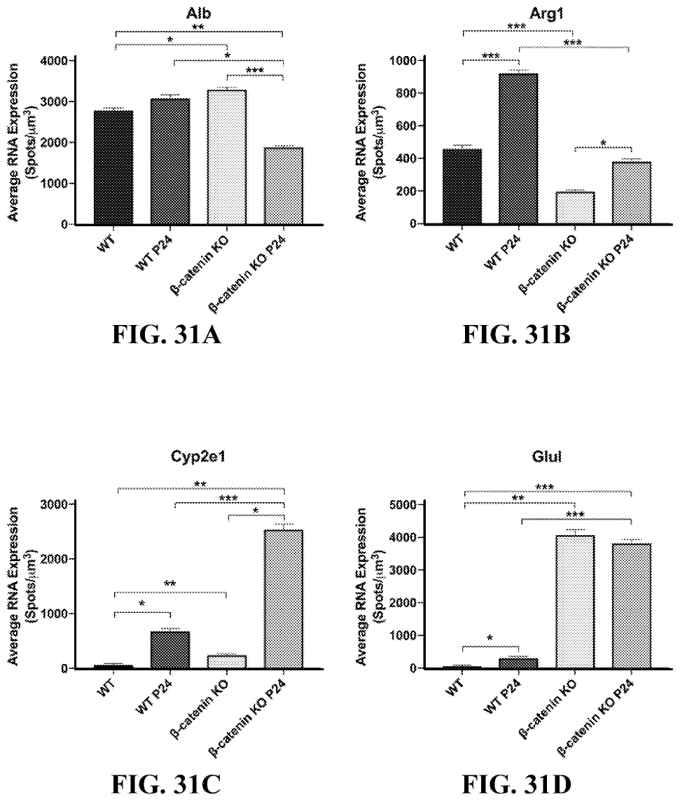

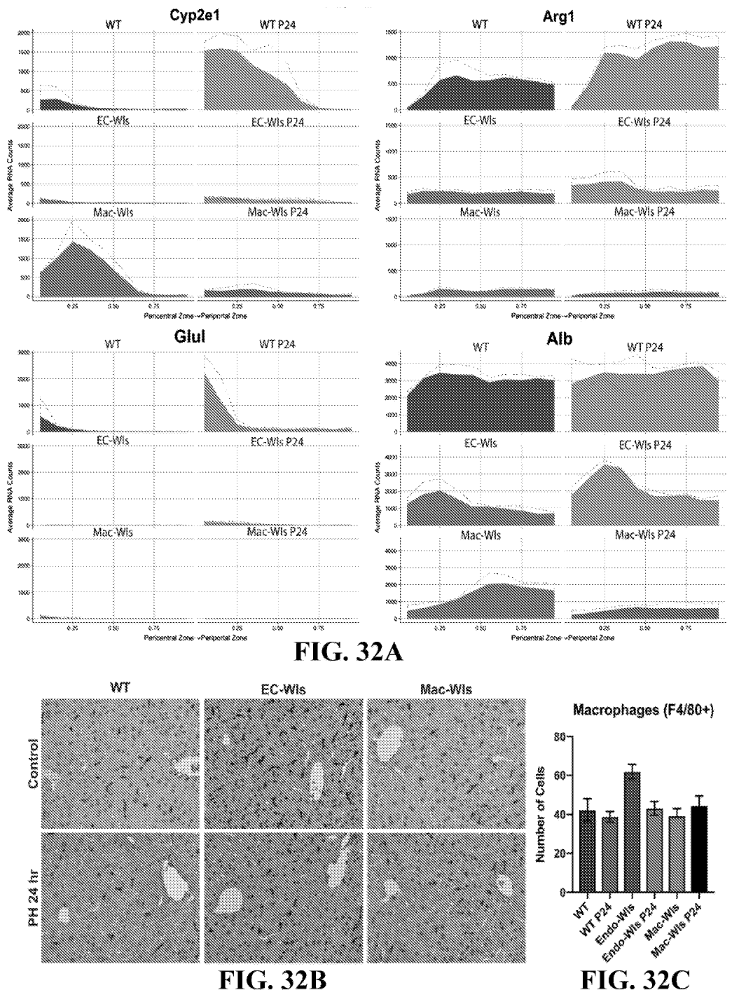

To identify if hepatic compensatory response following injury is also contributed by Wntβ-catenin signaling, Applicants evaluated hepatic gene expression via smFISH in hepatocyte-specific β-catenin KO mice after PH (FIG. 31). Functional compensation was β-catenin-dependent for select genes, such as Alb and Glu1. To identify the cellular source for secreted Wnt ligands responsible for functional compensation dependent on Wnt/β-catenin signaling, Applicants examined the contribution of both endothelium- and macrophage-derived Wnts using conditional Wntless (Wls) KO alleles that have intact β-catenin but lack proper Wnt processing and Wnt secretion from the respective cell populations (FIG. 22C)^{13,36}. Loss of either endothelium- or macrophage-derived Wnts, resulted in a down-regulation of hepatic function genes at baseline consistent with the role of Wnt signaling in the establishment and maintenance of hepatic gene expression during quiescence^{34,37,38} (FIG. 22D, 22E). Importantly, EC-Wls-KO mice are still able to transcriptionally compensate following PH. In contrast, loss of Wnt processing in macrophages of Mac-Wls-KO mice resulted in down-regulation of all examined hepatocyte genes in response to injury with the complete inability for transcriptional compensation (FIG. 22D). No change in resident hepatic macrophage numbers was observed (FIG. 32). Taken together, these data reveal that macrophage-derived Wnts are required for the observed functional compensation identified in this study, while endothelium-derived Wnts play a less significant role in this response.

### Discussion

The liver uniquely maintains complex metabolic function throughout injury and subsequent regeneration to enable survival of an organism^{39,40}. It has long been thought that the liver has sufficient functional reserve to maintain these functions through excess baseline capacity″, but the exact hepatic reserve capacity has been mostly a theoretical concept. Liver injury induces a regenerative response where functionally active hepatocytes are the major contributor to cellular regeneration. Turnover of hepatocytes in the uninjured organ is rather slow, with the entire liver being repopulated by new hepatocytes after ˜1 year^{12,45}. The liver can quickly respond to an acute insult, however, through activation of a regenerative response. Liver regeneration within the mouse model shows a peak of hepatocyte proliferation between 30-36 hrs for both PH and APAP-induced injury^{46,47}. Cell cycle genes are activated well before hepatocyte proliferation begins (priming phase) following injury^{39,40,48}. However, cell cycle inhibitors, such as p21 and p27, are concurrently up-regulated early in liver regeneration and block progression of hepatocytes into the cell cycle^{49,50}. It has been speculated that this co-expression of both stimulators and repressors of the cell cycle aides in the control of liver regeneration to a precise end point³⁹.

Here, Applicants describe a mechanism by which the liver has the ability to maintain essential liver function through transcriptional compensation when the proliferative response is delayed. Hepatocytes upregulate transcription of important liver genes, typically by adapting expression patterns extending beyond zonal boundaries. Importantly, many hepatocyte function genes are expressed predominantly in non-proliferating hepatocytes, while those cells that enter cell cycle by expression profile express hepatocyte function genes at lower levels. Collectively, our data define a novel compensatory phase following liver injury, thereby complementing previous studies which have elegantly established the field of liver regeneration by focusing on the hepatic proliferative response.

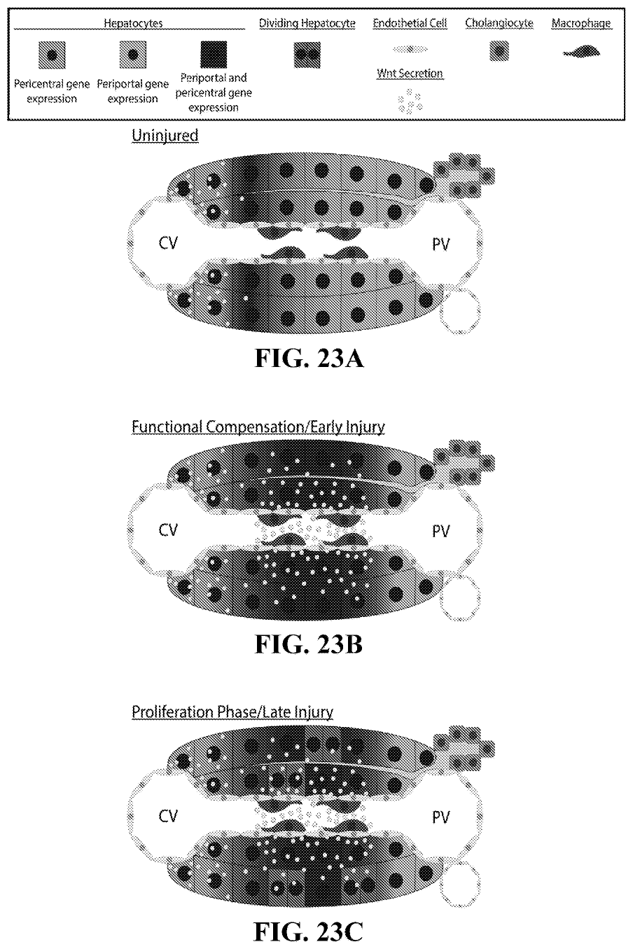

In addition, Applicants define a novel dual role for Wntβ-catenin signaling in liver regeneration: it not only promotes cell proliferation and cellular recovery, as shown in multiple studies^{10,12,21-24,13-20,} but it is also indispensable for functional compensation to maintain essential liver functions (FIG. 23). Applicants identified macrophages, but not endothelial cells, as a key source of secreted Wnts that enable transcriptional compensation. This is in contrast to other studies which have highlighted the contributions of endothelial-derived Wnts to maintenance of hepatic zonation as well as both endothelial and macrophage secreted Wnts to cellular proliferation^{13,23,37,38}. Applicants postulate that macrophages, which are responsible for broad inflammatory and immunologic functions⁵¹, are also essential for delivering Wnts locally throughout the entirety of the hepatic lobule (midzone and periportal areas) because of their ability to migrate and release Wnt ligands throughout the tissue. Our findings further highlight the potential of the Wnt/β-catenin pathway as a therapeutic target in acute liver failure and other liver pathologies, where maintenance of liver function is essential. Future studies will be needed to identify specific Wnt ligands to promote liver function, regeneration, and survival in regard to multiple pathologies that result in acute liver failure.

### Methods

### Animals

Three-month-old, male, C57BL/6J mice, purchased from Jackson Laboratories (Bar Harbor, Me., USA), were used in acute liver injury studies (APAP and PH). -catenin KO studies were conducted using Alb-cre^{+/-}; Ctnnb1^{flox/flox} mice. Wntless KO studies were conducted using Lyve1-cre^{+/-}; Wls^{flox/flox} (endothelial cell, EC-Wls) and Lyz2-cre^{+/-}; Wls^{flox/flox} (macrophages, Mac-Wls). All animals were housed in Association for Assessment and Accreditation of Laboratory Animal Care—accredited facilities at Brigham and Women's Hospital (Boston, Mass.) under a standard 12-hour light/dark cycle with access to chow and water ad libitum. The Institutional Animal Care and Use Committee at Brigham and Women's Hospital approved all studies.

### Acetaminophen (APAP) Exposure

Mice were fasted 12 hours before administration of APAP. APAP was dissolved in warm 0.9% saline, and mice were injected with 300 mg/kg APAP, i.p. Food was returned to the mice after APAP treatment. Mice were then used for isolation of primary hepatic cells for single cell RNA-sequencing or tissue harvest for further downstream analysis.

### Partial Hepatectomy

Partial hepatectomy surgeries were performed as previously described⁵². Mice were euthanized at 3 hrs, 48 hrs, and 5 days post-partial hepatectomy by cervical dislocation under isoflurane anesthesia and livers were harvested for downstream analysis. Further, mice were used for isolation of primary hepatic cells at 3 hrs, 48 hrs, and 5 days post-partial hepatectomy.

Isolation of Primary Hepatocytes and Non-Parenchymal Cells

Mouse hepatic cells were isolated by a modification of the two-step collagenase perfusion method⁵³. Cells were isolated from untreated (n=3 for each sex), APAP-treated mice (n=2 for each sex at 6, 24, 48, and 96 hours following APAP exposure), and mice subjected to partial hepatectomy (n=3; 3 hrs, 48 hrs, and 5 days). The digestion step was performed using Liver Digest Medium (Cat. #17703034; ThermoFisher Scientific; Pittsburgh, Pa., USA). Cell suspensions were used immediately for Seq-Well.

### Tissue Harvest

Untreated (n=3 for each sex) and APAP-treated mice (n=3 for 6, 24, 48, and 96 hours following APAP exposure and 3, 48, and 120 hours following PH) were euthanized by cervical dislocation following carbon dioxide exposure. A portion of liver tissue was fixed in 10% neutral buffered formalin for 48 hrs and further processed to obtain paraffin blocks and 5 μm thick sections. A portion of liver tissue was frozen in optimal cutting temperature (OCT) medium and used to obtain 10 μm fresh frozen sections.

### Library Preparation and Sequencing

Sequencing libraries were prepared from the single-cell suspension using the Seq-Well method as described in Gierahn et. al. 2017. Briefly, a microwell array was loaded with barcoded polyT mRNA capture beads (Chemgenes). Then 200 μl of media containing 15,000 single cells was loaded onto the array and allowed to settle into the wells by gravity. Membrane sealing, lysis, hybridization, reverse transcription, exonuclease digestion, second strand synthesis, PCR, and library construction by Nextera were all performed as previously described⁵⁴. Resulting libraries were quantified by Qubit and tape station (Agilent), and sequenced on an Illumina NextSeq 500 (UT and APAP samples, 2 arrays per run) or a NovaSeq (PH samples, 10 arrays per run) 30 cycle, paired end sequence reads, single 8 cycle index for NextSeq or dual 8 cycle indexes for NovaSeq.

### Single-Cell Sequencing Data Processing

Sequencing data was demultiplexed and aligned to mm10 with STAR aligner. Libraries were sequenced to an average depth of >48,000 reads per cell per sample. See Table 12 for additional sequencing and data quality metrics.

Barcodes with fewer than 400 genes were discarded from the genes x cells data matrix as non-cells, with 16,019 cells remaining. Data was log normalized and TPM-like (base 10,000) normalized and analyzed using the Seurat package in R⁵⁴. The resulting data displayed fairly even nGene and nUMI distributions across each sample type (FIG. 24H). Applicants performed a principal component analysis (PCA) and selected the top 13 principal components (PCs) for tSNE dimensional reduction. Applicants then performed shared nearest neighbors (SNN) clustering, and identified 14 distinct clusters in the data (FIG. 24C, 24D). Applicants calculated differential expression across the clusters using Wilcox test in the FindAllMarkers function in the Seurat R package and quantified expression of marker genes for known liver cell populations (FIG. 24D, 24E). Applicants identified nine high-quality hepatocyte clusters, separated by treatment condition; one low quality hepatocyte cluster with a high percent mitochondrial content and low nGene and nUMI; a kupffer cell cluster; a liver endothelial cell (LEC) cluster; a neutrophil cluster; and a mixed immune cluster, which appears to contain T cells, B cells and monocytes. Applicants calculated a hepatocyte signature score using AddModuleScore in Seurat over multiple highly expressed hepatocyte genes which span the lobule: Apoa1, Glu1, Acly, Asl, Cyp2e1, Cyp2f2, Ass1, Alb, Mup3, Pck1, G6pc, Fabp1.

In order to focus on hepatocyte responses, Applicants subsetted our data to include on the nine high-quality hepatocyte clusters. Following subsetting, Applicants observed a remaining few cells scoring low on the hepatocyte signature. Applicants filtered out any cells with a Hepatocyte Signature score less than 3 standard deviations below the average as non-hepatocytes (FIG. 24E). These non-hepatocyte cells originated primarily from the A6 sample, which dhad the largest immune infiltration in response to injury and the highest fraction on non-parenchymal cells in the total sample. The filtered non-hepatocytes are likely non-parenchymal cells incorrectly assigned to a hepatocyte cluster by SNN. Following these filtering steps, Applicants retained 10,833 high-quality hepatocytes for analysis.

### Single-Cell Sequencing Data Analysis (Hepatocyte Data)

Applicants performed dimensional reduction and clustering again on our filtered hepatocyte only dataset. Principal component 1 (PC1) describes 46.9% of and captures technical variation (nGene, nUMI) in the data (FIG. 25E). This is not surprising for a dataset comprised of a single cell type. Each of our treatment conditions scores similarly on PC1 (FIG. 25B). PC2 partly captures pericentral-periportal variation. Applicants identified zonally restricted genes in PC2 loadings (Cyp2e1, Cyp1a2, Gtsm3; Cyp2f2). Applicants also noted periportal-pericentral variation captured in PC4.

To more clearly visualize pericentral-periportal variation, Applicants scored cells on this pericentral periportal metric. To generate a list of pericentral genes, Applicants calculated gene-by-gene correlations with Cyp2e1, a canonical pericentral gene. To generate a pericentral gene list, Applicants selected genes positively correlated with Cyp2e1, and to generate a periportal gene list, Applicants selected genes negatively correlated with Cyp2e1 (Table 17). To generate a list of genes to be used for our signatures, Applicants considered all genes with a Cyp2e1 correlation >0.3 for PCHSig and a Cyp2e1 correlation <−0.3 for PPHSig. From the genes falling within this range of values, Applicants selected moderately expressed genes with large variability in expression across the dataset, removing lowly expressed genes and genes expressed in small numbers of cells. Positive correlations with Cyp2e1 range from 0.823 (Cyp2c29) to 0.356 (Ang); negative correlations with Cyp2e1 range from −0.569 (Cyp2f2) to −0.311 (Serpina12). The 0.3/−0.3 cutoff is more than 3 standard deviations above/below the mean of all gene correlations with Cyp2e1 (mean=−0.01280654; mean+3sd=0.2605311; mean−3sd=−0.2861442). Applicants then calculated the pericental hepatocyte (PCH) score and periportal hepatocyte (PPH) score using AddModuleScore for these genes. Applicants then confirmed that PCH Score and PPH Score are inversely correlated as expected. Applicants observe a pericental-periportal gradient across PC2 using these scores (FIG. 25A-25H). To generate a single score that captures pericentral-periportal character, Applicants subtracted the PCH Score from the PPH Score to create the PPH-PCH Score, in which pericentral hepatocytes will score negatively and periportal hepatocytes will score positively.

TABLE 17
Gene signatures used to define pericentral hepatocytes
(PCH) and periporatal hepatocytes (PPH).

| PCH genes | PPH genes |
|---|---|
| Lect2 | Hal |
| Rnase4 | Etnppl |
| Ang | Sds |
| Oat | Hsd17b13 |
| Gulo | Serpina12 |
| Cyp2c29 | Hsd17b13 |
| Cyp2e1 | Gas |
| Cyp2c50 | Aqp8 |
| Cyp2a5 | Cyp2f2 |
| Cyp2c37 | |
| Aldh3a2 | |

To better visualize the data, Applicants performed tSNE dimensional reduction. Hepatocytes from all samples look rather similar in lower PCs which describe shared variation, such as technical differences or cross-lobule variation, while the higher PCs capture inter-sample variation. Applicants calculated percent variation captured per PC and generated an elbow plot to determine the correct number of PCs to use in further analysis. Applicants selected the top 13 PCs to include in our analysis, which well separated samples by treatment condition and did not appear to be driven by technical artifacts. Applicants observe a technical gradient across each cluster (which is orthogonal to the pericentral-periportal gradient across each cluster), but the clusters themselves do not appear technically driven (FIG. 25H).

Heatmap genes were found using FindAllMarkers in Seurat, Wilcox test, min.percent=0.10, thresh.use=0.25. Mitochondrial and hemoglobin genes were removed from the list prior to heatmap plotting.

Shared and unique by injury model gene lists were assembled by combining DE results across all time points for each injury. Applicants ran differential expression using Wilcox test in the FindAllMarkers Seurat function between each treatment condition (A6, A24, A48, A96, PH3, PH48, PH120) individually and untreated (UT). Applicants then combined results across all time points within each injury model to obtain a list of all differentially expressed genes from any time point in APAP experiments (A6, A24, A48, A96) and PH (PH3, PH48, PH120). A small number of genes were up at one time point, but down at another. In these cases, the gene was retained in the list (up- or down-regulated) with the largest magnitude average log fold-change to capture to more significant change in expression.

Applicants ran pathway analysis on the composite DE results using the piano R package. Reference gene sets were downloaded from MSigDB (Broad Institute). Applicants used geneSetStat=“fisher”, adjMethod=“fdr”, and signifMethod=“geneSampling”. Applicants then parsed the results to identify shared and unique reference gene sets for each injury. Any reference gene set with a q-value greater than 0.05 was discarded as insignificant. Applicants then identified reference gene sets with significant overlaps with only APAP and with only PH composite DE results. To focus on truly unique responses, Applicants filtered out any reference gene set from the unique tables which had a q-value <0.2 for the other injury model. Applicants then identified shared responses by compiling all reference gene sets with a q-value of <0.05 in both APAP and PH. Selected reference gene set −log(q) are plotted in FIG. 20.

To identify cycling cells in the data, Applicants calculated Cell Cycle Score using AddModuleScore in Seurat over the cell cycle markers found in Tirosh et. al. 2015. Applicants classified cells with a Cell Cycle Score 2 standard deviations above the average as cycling cells (FIG. 4). To better compare cycling and non-cycling cells (CC and NC, respectively), Applicants subsetted the data to create a dataset containing all 51 CCs from the A24 condition and an equal number of NCs also from A24; similarly, Applicants created a dataset containing all 123 CC from PH48 and an equal number of NCs also from PH48. Pathway analysis was done on a DE result obtained from comparing 174 CC from A24 and PH48 against an equal number of NCs from these time points. Piano was run as described above. FIG. 21 plots −log(q) values for selected reference gene sets with a q value <0.05. Wnt Target Labbe Sig was calculated using AddModuleScore and the reference gene set LABBE_WNT3A_TARGETS_UP which was identified as significant in Piano gene set enrichment analysis.

### Immunohistochemical Analysis

Histology was performed by the histology core at Beth Israel Deaconess Medical Center using standard procedures and automated workflow. Samples were processed and embedded following fixation in 10% neutral buffered formalin for 48 hrs. Samples were embedded in paraffin and sectioned at 5 m thick. Immunohistochemistry was performed on a Leica autostainer (Leica Biosystems) with enzyme treatment (1:1000) using standard protocols. The antibody used for assessment of cell proliferation was PCNA (Cell Signaling, Cat. 13110, 1:800), and cell death was ApopTag Peroxidase In Situ Apoptosis Detection Kit (Millipore, Cat. #S7100). Macrophages were stained using the anti-F4/80 (Cell Signaling, Cat. 70076, 1:500). Sections were then counterstained with hematoxylin, dehydrated, and film cover slipped. Four representative images were captured per slide. TUNEL-positive area, PCNA-positive cells, and F4/80-positive cells were measured and averaged across the four images for each sample using Fiji.

Single Molecule Fluorescent In Situ Hybridization (smFISH)

smFISH was conducted using RNAscope technology (RNAscope Fluorescent Multiplex Kit; Cat. #320850; Advanced Cell Diagnostics; Neward, Calif., USA). Fresh frozen sections (10 m thick) were used following the manufacturer's guidelines. Probe sets were designed by the manufacturer and can be found at acdbio.com/catalog-probes. A 6×6 40× field was captured of a 10 μM z-stack (0.5 uM per slice). This resulted in multiple liver lobules available for analysis within a single section. Images were cropped to the size of a single liver lobule and cellular outlines were defined using CellProfiler⁵⁵. smFISH signal was then quantified using FISH-quant⁵⁶. Post-processing of mRNA detection was performed with custom-written Python scripts (available at bitbucket.org/muellerflorian/pyfishquant/). Pseudo-color images of transcript abundance were generated by setting the pixel values of each segmented cells to its corresponding transcript level. To determine the spatial expression gradients relatively to the central vein, Applicants manually outlined the vein as a polygon. This polygon served as a reference point to count RNAs in concentric rings. These counts were lastly renormalized by the ring area contained within the image.

### Statistical Analysis

Applicants calculated P values for shifts in gene expression or module scores using the Wilcox test, Bonferroni corrected for multiple testing. Gene set enrichment results in piano were calculated using Fisher's test and the gene sampling method and corrected by FDR. P values for average RNA expression (smFISH) and IHC counts (PCNA, F4/80, and TUNEL) were calculated using Welch's ANOVA tests.

The following includes references pertinent to example 3.
1. Kietzmann, T. Metabolic zonation of the liver: The oxygen gradient revisited. Redox Biol. 11, 622-630 (2017).
2. Budnitz, D. S., Lovegrove, M. C. & Crosby, A. E. Emergency Department Visits for Overdoses of Acetaminophen-Containing Products. AMEPRE 40, 585-592 (2011).
3. Couri, T. & Pillai, A. Goals and targets for personalized therapy for HCC. Hepatol. Int. 13, 125-137 (2019).
4. Michalopoulos, G. K. Hepatostat: Liver regeneration and normal liver tissue maintenance. Hepatology 65, 1384-1392 (2017).
5. Michalopoulos, G. K. Liver regeneration after partial hepatectomy: critical analysis of mechanistic dilemmas. Am. J. Pathol. 176, 2-13 (2010).
6. Michalopoulos, G. K. Liver regeneration. J. Cell. Physiol. 213, 286-300 (2007).
7. Michalopoulos, G. K. Principles of liver regeneration and growth homeostasis. Compr. Physiol. 3, 485-513 (2013).
8. Gonzales, K. A. U. & Fuchs, E. Skin and Its Regenerative Powers: An Alliance between Stem Cells and Their Niche. Dev. Cell 43, 387-401 (2017).
9. Gierahn, T. M. et al. Seq-Well: portable, low-cost RNA sequencing of single cells at high throughput. Nat. Methods 14, (2017).
10. Goessling, W. et al. APC mutant zebrafish uncover a changing temporal requirement for wnt signaling in liver development. Dev. Biol. 320, 161-74 (2008).
11. Ober, E. A., Verkade, H., Field, H. A. & Stainier, D. Y. R. Mesodermal Wnt2b signalling positively regulates liver specification. Nature 442, 688-91 (2006).
12. Wang, B., Zhao, L., Fish, M., Logan, C. Y. & Nusse, R. Self-renewing diploid Axin2(+) cells fuel homeostatic renewal of the liver. Nature 524, 180-5 (2015).
13. Preziosi, M., Okabe, H., Poddar, M., Singh, S. & Monga, S. P. Endothelial Wnts regulate β-catenin signaling in murine liver zonation and regeneration: A sequel to the Wnt-Wnt situation. Hepatol. Commun. 2, 845-860 (2018).
14. Ma, Y. et al. Wnt agonist stimulates liver regeneration after small-for-size liver transplantation in rats. Hepatol. Res. 46, E154-64 (2016).
15. Apte, U. et al. Beta-catenin activation promotes liver regeneration after acetaminophen-induced injury. Am. J. Pathol. 175, 1056-65 (2009).
16. Sodhi, D. et al. Morpholino oligonucleotide-triggered beta-catenin knockdown compromises normal liver regeneration. J. Hepatol. 43, 132-41 (2005).
17. Monga, S. P., Pediaditakis, P., Mule, K., Stolz, D. B. & Michalopoulos, G. K. Changes in WNT/beta-catenin pathway during regulated growth in rat liver regeneration. Hepatology 33, 1098-109 (2001).
18. Tan, X., Behari, J., Cieply, B., Michalopoulos, G. K. & Monga, S. P. S. Conditional deletion of beta-catenin reveals its role in liver growth and regeneration. Gastroenterology 131, 1561-72 (2006).
19. Apte, U. et al. Activation of Wnt/beta-catenin pathway during hepatocyte growth factor-induced hepatomegaly in mice. Hepatology 44, 992-1002 (2006).
20. Goessling, W. et al. Genetic interaction of PGE2 and Wnt signaling regulates developmental specification of stem cells and regeneration. Cell 136, 1136-47 (2009).
21. Huch, M. et al. In vitro expansion of single Lgr5+ liver stem cells induced by Wnt-driven regeneration. Nature 494, 247-50 (2013).
22. Gougelet, A. & Colnot, S. A Complex Interplay between Wnt/β-Catenin Signalling and the Cell Cycle in the Adult Liver. Int. J. Hepatol. 2012, 816125 (2012).
23. Ding, B.-S. et al. Inductive angiocrine signals from sinusoidal endothelium are required for liver regeneration. Nature 468, 310-5 (2010).
24. Birchmeier, W. Orchestrating Wnt signalling for metabolic liver zonation. Nat. Cell Biol. 18, 463-465 (2016).
25. Gebhardt, R. & Mecke, D. Heterogeneous distribution of glutamine synthetase among rat liver parenchymal cells in situ and in primary culture. EMBO J. 2, 567-70 (1983).
26. Varemo, L., Nielsen, J. & Nookaew, I. Enriching the gene set analysis of genome-wide data by incorporating directionality of gene expression and combining statistical hypotheses and methods. Nucleic Acids Res. 41, 4378-91 (2013).
27. Thirumoorthy, N., Manisenthil Kumar, K.-T., Shyam Sundar, A., Panayappan, L. & Chatterjee, M. Metallothionein: an overview. World J. Gastroenterol. 13, 993-6 (2007).
28. Oliver, J. R., Mara, T. W. & Cherian, M. G. Impaired hepatic regeneration in metallothionein-I/II knockout mice after partial hepatectomy. Exp. Biol. Med. (Maywood). 230, 61-7 (2005).
29. Jakovac, H. et al. Metallothionein expression and tissue metal kinetics after partial hepatectomy in mice. Biol. Trace Elem. Res. 114, 249-68 (2006).
30. Font-Burgada, J. et al. Hybrid Periportal Hepatocytes Regenerate the Injured Liver without Giving Rise to Cancer. Cell 162, 766-79 (2015).
31. Zhao, L. et al. Tissue repair in the mouse liver following acute carbon tetrachloride depends on injury-induced Wnt/β-catenin signaling. Hepatology (2019). doi:10.1002/hep.30563
32. Yang, J. et al. β-catenin signaling in murine liver zonation and regeneration: a Wnt-Wnt situation! Hepatology 60, 964-76 (2014).
33. Planas-Paz, L. et al. The RSPO-LGR4/5-ZNRF3/RNF43 module controls liver zonation and size. Nat. Cell Biol. 18, 467-79 (2016).
34. Leibing, T. et al. Angiocrine Wnt signaling controls liver growth and metabolic maturation in mice. Hepatology 68, 707-722 (2018).
35. Nejak-Bowen, K. N. & Monga, S. P. S. Beta-catenin signaling, liver regeneration and hepatocellular cancer: sorting the good from the bad. Semin. Cancer Biol. 21, 44-58 (2011).
36. Carpenter, A. C., Rao, S., Wells, J. M., Campbell, K. & Lang, R. A. Generation of mice with a conditional null allele for Wntless. Genesis 48, 554-8 (2010).
37. Preziosi, M., Poddar, M., Singh, S. & Monga, S. P. Hepatocyte Wnts Are Dispensable During Diethylnitrosamine and Carbon Tetrachloride-Induced Injury and Hepatocellular Cancer. Gene Expr. 18, 209-219 (2018).
38. Jiang, A. et al. Loss of Wnt Secretion by Macrophages Promotes Hepatobiliary Injury after Administration of 3,5-Diethoxycarbonyl-1, 4-Dihydrocollidine Diet. Am. J. Pathol. 189, 590-603 (2019).
39. Fausto, N. Liver regeneration. J. Hepatol. 32, 19-31 (2000).
40. Su, A. I., Guidotti, L. G., Pezacki, J. P., Chisari, F. V & Schultz, P. G. Gene expression during the priming phase of liver regeneration after partial hepatectomy in mice. Proc. Natl. Acad. Sci. U.S.A 99, 11181-6 (2002).
41. Coexistence of C/EBPα, β, growth-induced proteins and DNA synthesis in hepatocytes during liver regeneration. Implications for maintenance of the differentiated state during liver growth. J. Clin. Invest. 96, 1351-1365 (1995).
42. Haber, B. A., Mohn, K. L., Diamond, R. H. & Taub, R. Induction patterns of 70 genes during nine days after hepatectomy define the temporal course of liver regeneration. J. Clin. Invest. 91, 1319-26 (1993).
43. Haber, B. A. et al. High levels of glucose-6-phosphatase gene and protein expression reflect an adaptive response in proliferating liver and diabetes. J. Clin. Invest. 95, 832-41 (1995).
44. Haber, B., Naji, L., Cressman, D. & Taub, R. Coexpression of liver-specific and growth-induced genes in perinatal and regenerating liver: attainment and maintenance of the differentiated state during rapid proliferation. Hepatology 22, 906-14 (1995).
45. Planas-Paz, L. et al. The RSPO-LGR4/5-ZNRF3/RNF43 module controls liver zonation and size. Nat Cell Biol 18, 467-479 (2016).
46. Nevzorova, Y. A., Tolba, R., Trautwein, C. & Liedtke, C. Partial hepatectomy in mice. Lab. Anim. 49, 81-8 (2015).
47. Bhushan, B. et al. Pro-Regenerative Signaling after Acetaminophen-Induced Acute Liver Injury in Mice Identified Using a Novel Incremental Dose Model. Am. J. Pathol. 184, 3013-3025 (2014).
48. Albrecht, J. H. & Hansen, L. K. Cyclin D1 promotes mitogen-independent cell cycle progression in hepatocytes. Cell Growth Differ. 10, 397-404 (1999).
49. Albrecht, J. H. et al. Involvement of p21 and p27 in the regulation of CDK activity and cell cycle progression in the regenerating liver. Oncogene 16, 2141-50 (1998).
50. Bhushan, B. & Apte, U. Liver Regeneration after Acetaminophen Hepatotoxicity Mechanisms and Therapeutic Opportunities. Am. J. Pathol. 189, 719-729 (2019).
51. Abdullah, Z. & Knolle, P. A. Liver macrophages in healthy and diseased liver. Pflugers Arch. 469, 553-560 (2017).

### Example 4—High Fat Diet scRNA-Seq Captures Many Cell Types Across Tissues

To profile cellular responses to pro-obesity HFD, Applicants performed Seq-Well on mice maintained on a HFD (60% of calories from fat) as described in Beyaz et. al.¹ for six months. Diet-induced cellular changes are likely in progress by six months, with mice progressing to more severe manifestations of obesity-associated metabolic changes and gastrointestinal disease by around nine to 14 months. Obesity is linked to cancer and inflammation in both the gut and liver; therefore, Applicants profiled samples from multiple gastrointestinal and complementary immune sites to gain a fuller picture of the effects of HFD spanning multiple organs.

Samples from peripheral blood (PB), bone marrow (BM), spleen (Sp), liver hepatocyte-enriched (Hep), liver non-parenchymal-enriched (NPC), proximal small intestine (Prox), distal small intestine (Dis) and Colon (Col) were processed to single cell suspension and loaded onto a Seq-well array (Table 18, Methods). Prior to loading, crypts from proximal small intestine, distal small intestine and colon were isolated, dissociated into a single cell suspension and sorted into CD45+ and EPCAM+ populations to enrich for immune cells in the sample. The sorted populations (20,000 EPCAM+, 5,000 CD45+) were mixed together and loaded onto an array. Libraries were then prepared and sequenced on a Nova-Seq.

TABLE 18

| | nGene | nUMI | nCell filter |
|---|---|---|---|
| CD2Col | 2212 | 4889 | 1737 |
| CD2Dis | 2250 | 6129 | 1332 |
| CD2Hep | 2531 | 15127 | 1328 |
| CD2NPC | 670 | 2639 | 1326 |
| CD2PB | 453 | 1019 | 449 |
| CD2Prox | 4090 | 13068 | 1756 |
| CD4BM | 3406 | 10330 | 1868 |
| CD4Col | 2997 | 9707 | 1241 |
| CD4Dis | 3740 | 11319 | 1607 |
| CD4Hep | 239 | 1786 | 335 |
| CD4NPC | 464 | 1878 | 742 |
| CD4PB | 1755 | 5002 | 1075 |
| CO4Prox | 3693 | 10280 | 1965 |
| CD4Sp | 2870 | 7767 | 1543 |
| HF2Col | 2435 | 8610 | 935 |
| HF2Dis | 3309 | 10264 | 1292 |
| HF2Hep | 681 | 4713 | 964 |
| HF2NPC | 786 | 2763 | 1371 |
| HF2PB | 1409 | 5685 | 878 |
| HF2Prox | 2471 | 8137 | 1170 |
| HF3BM | 1757 | 4275 | 1804 |
| HF3Col | 646 | 1917 | 608 |
| HF3Dis | 1251 | 2748 | 1279 |
| HF3Hep | 216 | 837 | 231 |
| HF3NPC | 189 | 899 | 342 |
| HF3PB | 675 | 1393 | 745 |
| HF3Prox | 2749 | 6316 | 1813 |
| HF4Col | 3455 | 9932 | 1296 |
| HF4Dis | 3551 | 9690 | 1961 |
| HF4Hep | 540 | 1700 | 1347 |
| HF4NPC | 458 | 1696 | 960 |
| HF4PB | 1973 | 4497 | 1622 |
| HF4Prox | 3416 | 8702 | 1683 |

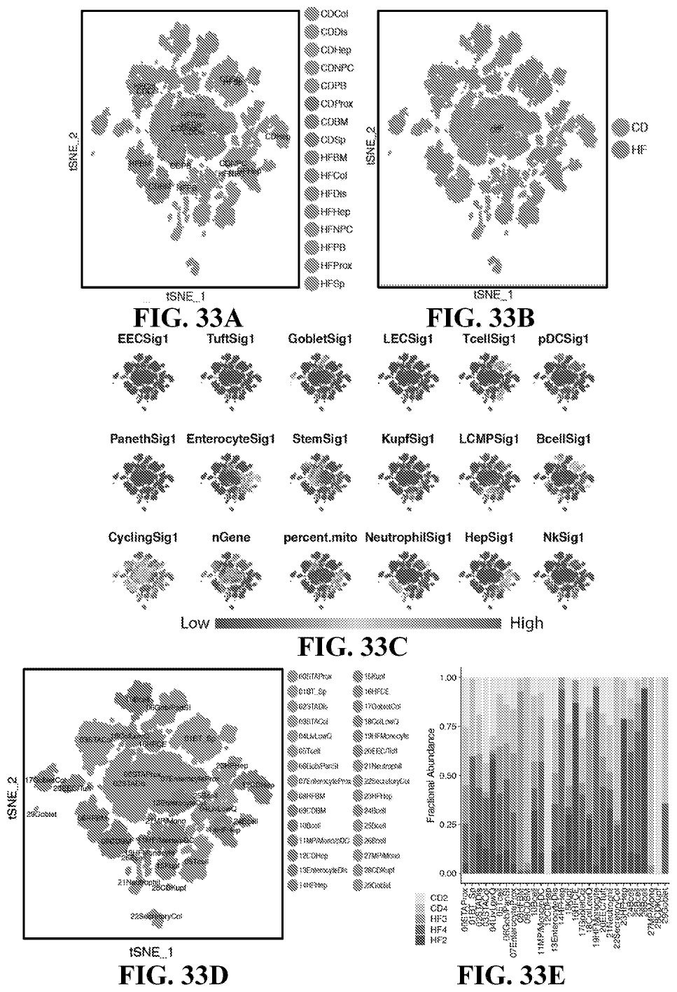

Following data processing and filtering, Applicants obtained a total of 42,684 cells. To visualize the data, Applicants performed dimensional reduction by Principal Components Analysis (PCA) and t-Stochastic Neighbor Embedding (t-SNE). Applicants identified groups of similar cells using Shared Nearest Neighbor (SNN) clustering, and generated module scores from marker genes highly expressed in various cell types to identify the cell type present in each cluster (FIG. 33A-33D, Methods). Applicants identified several clusters and multiple types of intestinal cells: stem/transamplifying (STA), Enterocyte, Enteroendocrine (EEC), Goblet, Paneth and Tuft. STA and Enterocyte clusters separate mainly by point of origin: proximal, distal, or colon (FIG. 33A-33D). Applicants observe immune cell (B cell, T cell) clusters populated by cells from many different compartments. Liver-resident Kupffer and hepatocyte clusters emerge, with clear separation by diet condition in hepatocytes. Finally, Applicants identified bone marrow- and spleen-specific clusters of immature immune cells.

Table 18 provides samples processed from two control diet (CD2, CD4) and three high fat diet (HF2, HF3, HF4) mice. Samples were prepared from bone marrow (BM), colon (Col), distal small intestine (Dis), liver hepatocyte-enriched (Hep), liver NPC-enriched (NPC), peripheral blood (PB), proximal small intestine (Prox) and spleen (Sp). Due to technical challenges not all samples were obtained from all mice. Number of genes (nGene) and number of unique molecular identifiers (nUMIs) were calculated for each sample over all events called in alignment. Number of cells remaining after filtering for >500 transcripts and >200 genes (nCell filter) reported for each sample.

Applicants applied quality metrics: number of genes (nGene), number of unique molecular identifiers (nUMI, number of RNA molecules captured) and percent mitochondrial content (percent.mito; NB high mitochondrial content can indicate cell membrane disruption from excessively harsh processing and diminished data quality); and Applicants identified two low quality clusters mainly originating from colon and from liver which Applicants omit from further analysis (FIG. 33C, 33D). Applicants noted lower quality in the HFD hepatocyte clusters relative to other cell types. Cells isolated from the livers of HFD animals are incredibly delicate, likely due to increased volume of fats, and strongly encapsulated within the more fibrotic tissue found in HFD. This increased tissue fibrosis and larger gross liver size necessitated harsher profusion and digestion conditions, as well as longer treatment time to liberate single cells for analysis. It has also been noted in the literature that hepatocyte mitochondrial content can be very high and that hepatocytes appear highly susceptible to damage from processing. It has been postulated that these large fragile cells' membranes are more easily disrupted which may further inflate mitochondrial content due to loss of cytosolic mRNAs. Here, metabolic changes induced by HFD may also contribute to shifts in mitochondrial gene expression.

### HFD-Induced Changes in the Gut

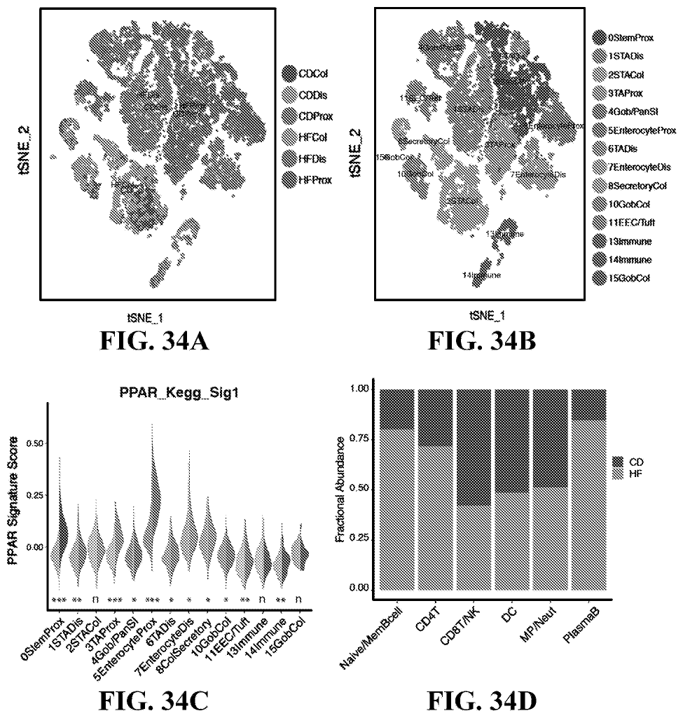

To more clearly assess diet-induced shifts in the gut, Applicants subsetted the dataset to include only samples that originated in the proximal, distal or colonic regions, and filtered out the low-quality colon cluster and the irreproducible HF2 Proximal cluster (Methods). Applicants performed dimensional reduction and SNN clustering again on this subsetted data, and assigned cell type identities to each cluster as Applicants did for the full dataset. Each cluster is populated with cells from HFD and CD mice, yet Applicants noticed some diet-based variation within clusters, especially in the enterocyte clusters (FIG. 34A, 34B).

Previous work has reported that PPAR signaling drives differences between HFD and CD intestinal cells at 9-14 months on the diet. Applicants calculated a PPAR signaling score over the gut cells to determine whether this pathway is already activated in the intestines at 6 months on HFD in each of the cell type clusters captured (Methods). Indeed, in some of the cell type clusters Applicants observe upregulation of PPAR target genes (KEGG_PPAR_SIGNALING_PATHWAY, Broad MSigDB) in the HFD compared to the CD condition, as previously reported¹ (FIG. 34C). Interestingly, Applicants found the strongest upregulation of the PPAR program in the proximal enterocyte cells (d=2.13), upregulation in the proximal stem (d=1.94) and transamplifying cells (d=1.94), and little to no upregulation in the distal and colon samples as well as for cell types other than enterocyte/transamplifying/stem (d=0.629 to 0.03) (FIG. 34C). These patterns are represented in each of the multiple mice in this dataset (data not shown). This supports the report by Beyaz and colleagues of an HFD-induced increase in PPAR target gene expression in the small intestine and colon at around one year on HFD¹. the data show significant upregulation of PPAR targets has begun by six months on HFD in the proximal region, but suggests that changes in the distal or colonic regions may involve lower levels of PPAR activation, occur more slowly, or involve pathways other than PPAR at this time point.

To explore other pathways which may be involved in the distal small intestine and colon Applicants performed pathway analysis with Ingenuity Pathway Analysis (IPA) from Qiagen. Applicants found decreased activity of RB1, a tumor suppressor, in HFD enterocytes from the proximal region (z-score −2.945, p-value 5.51e-03) and stem/transamplifying cells from the colon (z-score −3.537, p-value 1.41e-11). Applicants also found upregulation of RELA, involved in NF-κB signaling and inflammation in colon stem/transamplifying HFD (z-score 2.779, p-value 1.85e-05).

Next, the gut immune cell cluster was subsetted to further refine the cell type cluster assignments by iterative clustering. Applicants identified several cell types, including B cells (naïve/memory and plasmablast) CD4+T cells, CD8+T cells/NK cells, dendritic cells (DC), macrophages and neutrophils, and noticed fluctuations in the frequencies of these subsets between HFD and CD (FIG. 34D). Importantly, each of the gut samples was sorted prior to loading onto the Seq-Well array to enrich for immune cells, and each array was loaded with the same 1:4 ratio of CD45+ to EPCAM+ cells (Methods). However, Applicants ultimately obtained inconsistent numbers of immune cells in each of the samples. This is likely due to a combination of variable relative viability among cell types and sequencing depth. The absolute number of immune cells is variable, and the ratio of immune to non-immune ranges from 14% immune in HF2 to 2% in HF4. There appears to be a trend of more deeply sequenced samples (CD2, HF2) containing more immune cells, suggesting deeper sequencing of samples from experiments 3 and 4 may increase immune cell numbers. The immune cells that make up the immune component in each sample vary considerably in their fractional abundance of immune cell types between HF and CD. The HFD samples have a much higher fraction of B cells while the CD immune population contains more T cells, dendritic cells, and macrophages. This variability may represent an infiltration of B cells or efflux of T cells and macrophages in HFD, or the reverse in CD. Since the protocol accepts a set number of cells an input, an increased infiltration of one cell type will result in a decrease in the fractional abundance of others in the data, making absolute abundance difficult to determine the data suggests some shift in immune composition, but additional experiments, such as flow analysis, are needed to quantitatively ascertain the abundance of various immune subsets in HFD and CD guts.

### HFD-Induced Changes in the Liver

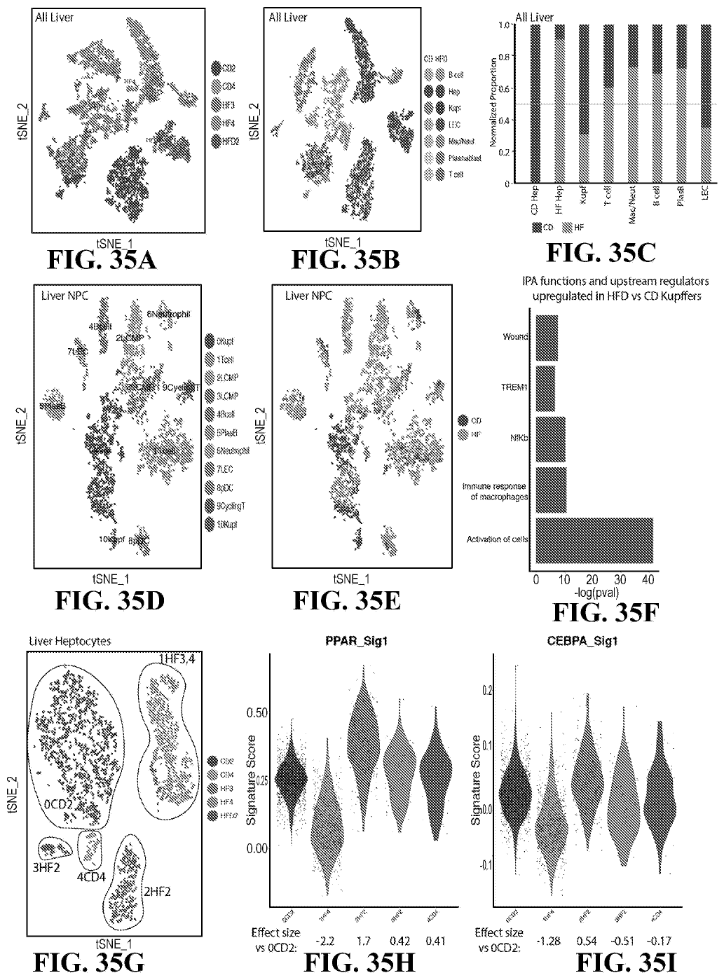

Obesity is known to increase risk for both intestinal and liver disease. The HFD mice in this study do begin to develop liver problems by 6 months on the diet and, in some cases, progress to spontaneous HCC at later time points. To dissect and study HFD-induced transformations in the liver at single-cell resolution, Applicants applied Seq-well to liver samples from HFD and CD. Biological changes in the HFD liver make hepatocytes more sensitive to processing due to fat accumulation while, at the same time, making the liver larger, more fibrotic and difficult to dissociate, presenting challenges in processing. For these reasons, the HFD liver data is of lower quality (lower nGene, lower cell number, higher percent mitochondrial content) than CD liver, but still interpretable (Table 18, FIG. 35A). Applicants have already made several adjustments to the protocol to improve data to this point (Methods), but future iterations may make additional adjustments to improve HFD liver data quality.

Applicants subsetted the dataset to include only samples originating in the liver, performed dimensional reduction and reclustering (Methods). Applicants identified expected liver cell types: Hepatocytes, liver endothelial cells (LECs), Kupffer cells, macrophage/monocytes, pDCs, T cells, B cells and neutrophils (FIG. 35A, 35B). Intriguingly, the hepatocytes form distinct clusters separating by diet, with some diet-based shifts evident in other cell types as well (FIG. 35C).

Applicants performed iterative clustering over the non-parenchymal cells of the liver to gain greater resolution in calling the cell types represented. Applicants identified Kupffer cells, liver capsule macrophages (LCMP), pDCs, Neutrophils, liver endothelial cells (LEC), B cells, and T cells (FIG. 35D). Kupffer cells were the most plentiful cell type in the NPC dataset and appear to separate slightly by diet condition (FIG. 35E). Applicants ran IPA over genes differentially expressed between HFD and CD Kupffer cells. Within “Diseases & Functions”, Applicants found upregulation in HFD of “Immune response of macrophages” (z-score 1.778, p-value 1.65e-11), “Activation of cells” (z-score 2.294, p-value 2.42e-42) and “Wound” (z-score 2.219, p-value 1.97e-08). HFD also showed upregulation of the activity of several upstream regulators such as pro-inflammatory NF-κB (z-score 2.179, p-value 3.98e-11) and TREM1 (z-score 2.938, p-value 2.31e-07). Taken together, these results present HFD kupffer cells as more activated and more inflammatory than in CD (FIG. 35F).

To specifically analyze cellular responses in the hepatocyte data, Applicants selected hepatocyte clusters, filtered on a mitochondrial content cutoff of 50%, as has been reported previously¹⁸, and performed iterative clustering over the remaining cells (Methods). Applicants identified a large cluster of hepatocytes originating mainly from CD2, a large cluster from HF4 and HF3, a smaller cluster from HF2, a small cluster from CD4 and another small cluster from HF2 (FIG. 35G). Applicants performed differential expression between the HFD and CD hepatocytes and ran pathway analysis on the resulting differentially expressed genes through IPA. Results from IPA “Diseases & Functions” identifies upregulation of “Liver steatosis” (z-score 3.522, p-value 2.93e-21), “Hepatic steatosis” (z-score 3.522, p-value 2.93e-21), “Inflammation of liver” (z-score 1.857, p-value 1.50e-09), Oxidative stress (z-score 3.657, p-value 1.10e-11) and “Accumulation of cholesterol” (z-score 2.320, p-value 3.95e-07), as well as a decrease in “Synthesis of lipid” (z-score −3.501, p-value 6.89e-36) in the HFD compared to CD hepatocytes. IPA upstream regulators show a downregulation in HFD of activity of SREPF2 (also known as Srebp2, z-score −4.883, p-value 1.67e-24), a transcription factor responsible for activating synthesis and uptake of cholesterol and fatty acids. This aligns well with the expected biology of the HFD liver, confirming that Applicants have captured interpretable data.

Next, Applicants asked whether activation of the PPAR pathway in the HFD condition occurs in the liver as it does in the gut. Applicants found PPAR target genes upregulated in HF2 compared to CD2, but down regulation of PPAR in HF3 and HF4 compared to CD2 (FIG. 35H). Samples from experiment 2 were resequenced to achieve greater sequencing depth, while some samples from experiments 3 and 4, especially HF4Hep, are undersequenced, possibly affecting PPAR target gene expression sensitivity. Applicants will address this possibility by resequencing these samples to improve depth. Alternatively, PPAR activation may occur in some HFD mice, but not others at the six-month time point. In search of other regulators which may contribute to HFD-induced changes in the liver, Applicants combed the Upstream Regulator IPA results for potential drivers of HFD-induced changes in the liver. IPA identifies significant downregulation in HFD of activity of RB1 (z-score −5.82, p-value 8.43e-14), a transcription factor with tumor suppressive function²¹, and down regulation, particularly in HF4, of activity of CEBPA (z-score −4.749, p-value 4.93e-13), a transcription factor involved in cell cycle regulation, lipid and glucose metabolism in the liver, and leptin expression and body weight homeostasis, whose function is known to be suppressed in HCC and other types of liver disease (FIG. 35I). IPA also identified upstream regulators whose function increased under HFD conditions, including NCOR1 (z-score 2.6, p-value 2.85e-10), which can contribute to thyroid hormone resistance, and hormonal and metabolic changes. Additional work is needed to further explore and validate the potential contributions of these pathways to HFD-induced changes in the liver.

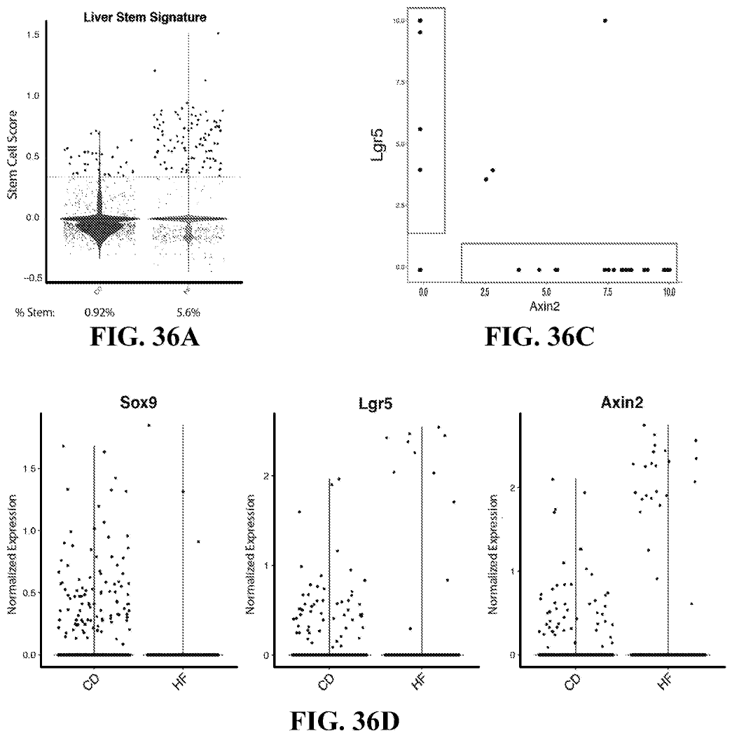

Changes in proliferative potential and “sternness” can prime cells to grow in dysregulated ways, possibly leading to cancer. Although a dedicated liver stem cell population has never been definitively identified¹², Applicants postulate that a subset of hepatocytes may activate stem function in response to HFD and progress toward the development of HCC. Thus, Applicants scored hepatocytes on a stem cell signature to identify changes is stem-like expression and searched for changes in these stem-like cells which may lead to HCC. More specifically, Applicants scored hepatocytes on expression of stem marker genes Lgr5, Axing, Sox9, Asc12, Tbx3 and Gkn3 (Methods). Applicants identified hepatocytes which have activated a stem cell program as cells scoring at least two standard deviations above average. (FIG. 36A). A much higher percentage of HFD hepatocytes score as stem cells than CD hepatocytes (5.6% vs 0.92%), supporting the notion that HFD may increase stemness in the liver, similarly to what has been reported in the gut. High expression of the stem signature in hepatocytes was driven mainly by expression of Sox9, Lgr5 and/or Axin2. HFD appears to dysregulate expression of these genes, with suppressed expression of the stem gene Sox9 and increased expression of Lgr5 and Axin2 in HFD compared to CD (FIG. 36B). Interestingly, Lrg5 and Axin2, the stem genes most highly expressed in HFD hepatocyte stem cells, are expressed largely mutually exclusively, in contrast to the gut where they are coexpressed (FIG. 36C). Many of the genes correlated with Lgr5 expression and Axin2 expression in the hepatocyte dataset are involved in cytokinesis and cell cycle pathways, supporting the notion that cells expressing these genes may possess increased proliferative potential. Further identification and characterization of changes in stemness within hepatocytes will serve to pinpoint the cellular origins of HCC, which remain poorly defined.

### Liver Organoids

Organoids can serve as a useful model system for evaluating perturbations in vitro and assessing the stemness of input samples. In the intestine, HFD samples possessed greater capacity to form and grow organoids, a characteristic of their enhanced stemness. This same characteristic may enhance their ability to progress to tumors. Here, Applicants pilot a recently published hepatocyte organoid protocol with the HFD and CD hepatocyte-enriched samples to assess their relative abilities to form and grow organoids.

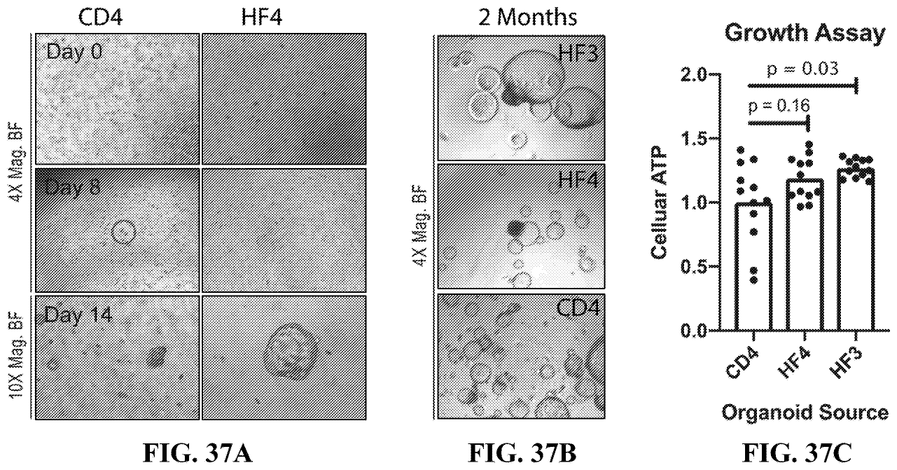

Applicants seeded organoids in matrigel and noted clear morphological differences between the HFD and CD hepatocytes, suggestive of their biology. Despite efforts to seed equal numbers of cells for both conditions, in this pilot experiment, the HFD was seeded much less efficiently than the CD (more cells matrigel at day 0 for CD than HFD) (FIG. 37A). As expected, only a few organoids formed and grew very slowly. Applicants continued to grow the organoids under the prescribed conditions and noted heterogeneous morphology. Some organoids appeared solid and bumpy or branched (the hepatocyte organoid morphology) while others appeared cystic and spherical (the cholangiocyte or biliary morphology). Over time, the cultures shifted to contain all cholangiocyte morphology organoids in all samples (FIG. 37B). Some individual hepatocyte morphology organoids shifting to cholangiocyte morphology in culture; additionally, the culturing conditions apply a selective pressure in favor of the faster growing cholangiocyte-like organoids. The large, spherical cholangiocyte organoids may break apart during passaging and seed more of these organoids in the new matrigel. The published protocol did not report this cholangiocyte shift phenomenon, which is likely a product of the much older mice used in the study (a few weeks old in the published protocol versus seven to eight months in the experiments here). Bidirectional ability for biliary and hepatic cells to regenerate one another has been reported in vivo, suggesting the possibility of interconverting between these cell types under proper organoid culture conditions (which have yet to be determined). By around two months in culture, the liver organoids began growing much more rapidly, likely due to adaptation to culture conditions and selective pressure for cells able to grow rapidly in vitro. Applicants performed an ATPase growth assay and detected a subtle enhanced growth phenotype in the HFD hepatocyte-derived organoids (CD4 vs HF4 p-value=0.16, CD4 vs HF3 p-value=0.03, ANOVA with multiple testing correction) even after months in culture (FIG. 37C). Additionally, Applicants performed Seq-Well on the liver organoids to determine how faithfully they recapitulate the transcriptional profiles of the hepatocytes from the same animals which were immediately profiled (HF3, HF4, CD4 hepatocyte-enriched samples). Data processing and analysis for this experiment are ongoing.

### Follow-Up and Ongoing Experiments

The current pilot dataset analysis has characterized many cell types from multiple compartments, identified biologically meaningful shifts in transcript expression, and nominated pathways which may participate in driving these changes; yet, more work remains to be done to further explore and validate these observations.

In agreement with earlier work from the Yilmaz Lab, the present work observes upregulation of PPAR in HFD intestinal samples. In the data, PPAR upregulation occurred mainly in the proximal region. Applicants also noted significant upregulation of PPAR activity in enterocyte cells from the proximal region, not mentioned in previous work which was focused on stem and progenitor cells. Importantly, the experiments were performed at an earlier time point than in the published work (6 months vs 9-14 months on diet), thus representing earlier initiation of PPAR activation at least in some regions. Additional experiments, such as the organoid, and imaging experiments described in the earlier publication¹, could be performed at the earlier time point used here to validate the findings.

Beyond the gut, Applicants also observe diet-induced changes in the liver. While upregulation of PPAR activity was inconsistent in the liver, it remains a possible driver of liver changes in need of further investigation. In the gut, treatment with a PPAR agonist recapitulated the effects of HFD, and a similar experiment could be performed in the liver or liver organoids to determine whether a PPAR agonist can recapitulate HFD effects in the liver as well. In addition to PPAR, Applicants identified other pathways which may be activated or deactivated in the liver. Modulation of these upstream regulators through agonists or inhibitors will provide insight into the role of these pathways in driving changes in the HFD liver.

In both liver and gut, Applicants noted possible shifts in immune composition, but these shifts are difficult to interpret in existing data due to confounding factors (CD45 sorting in gut samples, technical effects, undersequencing of some samples). Flow analysis to quantify the abundance of immune subsets in the liver and gut will determine whether immune populations, such as B cells in the gut or macrophages in the liver, infiltrate into these organs in HFD. Additionally, Applicants have sequencing data from reference immune sites in the peripheral blood, bone marrow and spleen. Comparing between immune cells within the liver or gut to immune cells outside these organs will reveal how these immune cells respond to the inflammatory or oncogenic environment in the HFD gastrointestinal system. “Spill over” genes from the free RNAs in the media in cell loading complicate comparisons of a given cell type across sample types. Application of a computational tool such as SoupX for background correction to remove this contamination will be needed to properly compare immune cells across organs.

Infiltrating immune cells may travel between the liver and gut and support cross-talk between gastrointestinal and immune sites. Further analysis of bone marrow, peripheral blood and spleen samples may identify immune responses to HFD outside the GI system if such responses exist. In the dataset HFD and CD bone marrow samples do cluster separately, but technical differences in sequencing depth dominate the differences between the HFD and CD data in this compartment. After deeper sequencing, Applicants will be equipped to better compare these samples.

Analysis of sequenced liver organoids is ongoing and when complete will determine how closely the organoids recapitulated the biology of the hepatocytes from the HFD and CD livers. Results may guide further optimization of the organoid culturing protocol. Future organoid experiments will work toward better normalizing seeding efficiency to facilitate comparison of organoid forming efficiency between diets. Ideally, Applicants will be able to grow organoids with a consistent hepatocyte phenotype and controlled seeding from HFD and CD samples. Withdrawal of some growth factors, such as WNTs, from organoid culture during the first few days could select for cells already primed for growth or proliferation in the in vivo environment and may confer a stronger growth advantage to the HFD-derived organoids. If so, this would demonstrate the enhanced ability of HFD hepatocytes to survive and grow in vitro and possibly form tumors in vivo. In the published work with gut organoids from HFD and CD, culturing CD-derived organoids with lipids recapitulated the effects of HFD. Similarly, culturing CD hepatic organoids with these lipids will determine whether these fats also affect these liver organoids in analogous ways.

Extensions of this work to future projects may include building a dataset over a full-time course of 3, 6, 9 and 12 months, repeating experiments with female mice to explore sex-differences in HFD responses, and extending the work to human samples are discussed in detail in Chapter 6. The work described here and these extensions will deepen the understanding of the effects of obesity and diet on the gastrointestinal system and development of diet-induced cancer, and point toward potential therapeutic targets. Further validation and development of these candidate targets may one day lead to improved treatment options for NASH, HCC, and intestinal cancers.

### Methods

### Mice

Mice were maintained on a high fat diet (HFD) or control diet (CD) for 6 months, as described previously. Liver samples (hepatocyte-enriched and NPC-enriched) were obtained as described in Example 3. Intestinal samples (proximal small intestine, distal small intestine and colon) were processed to enrich for crypts, then dissociated to single cell suspensions. Single-cell suspensions were sorted on a Sony SH800 flow sorter into CD45+ (immune) and Epcam+ to increase input of immune cells. One array was loaded for each intestinal sample with a sorted population of 5,000 immune cells and 20,000 Epcam+ cells. Counting of sorted populations showed that only about half as many cell as expected are in the sorted populations, so the arrays were loaded with close to the target of 15,000 cells.

### Library Preparation and Sequencing

Samples were run according to the Seq-Well version 2 protocol with second strand synthesis with the following adjustments: increased loading from 10,000 to 15,000 cells, media for loading and sealing was changed from RPMI to Hepatocyte media for liver samples and crypt media for gut, cell loading time was increased from 5 minutes to 15-20 minutes for liver samples and 10 minutes for all other samples. Applicants note that prompt processing of all samples, especially liver samples, is essential to obtain quality so each sample was processed as soon as it was ready, rather than waiting for several samples to run in parallel.

Libraries were sequenced on a Nova-Seq (Illumina) at 12 libraries per run. Undersequenced libraries from experiment 2 were sequenced again to improve sequencing depth. Resequencing of some samples in Experiments 3 and 4 to increase sequencing depth is still needed. Average nGene <1000 may indicate undersequencing. Sequencing output was aligned to mm10 by STAR aligner. Events with fewer than 500 transcripts captured were discarded as non-cells. Remaining cells were filtered on >200 genes expressed. More stringent filtering results in excessive loss of HFD hepatocytes. This filtering does allow some low-quality cells/events into the dataset, but these cells readily cluster together and are filtered out from subsequent analysis.

### Sequencing Data Analysis

Filtered data was analyzed using primarily the R package Seurat version 2 from the Satij a Lab. Applicants performed dimensional reduction by Principal Components Analysis (PCA) over the dataset. Applicants selected significant PCs from the Elbow Plot and performed t-Stochastic Neighbor Embedding (tsne) over the selected PCs and Shared Nearest Neighbor (SNN) clustering over those same PCs. Differential expression was performed using the “FindMarkers” function and the Wilcoxon statistical test.

Cell type signatures were created using the AddModuleScore function in Seurat and a list of marker genes for each expected cell type. These module scores were used to assign cell types to SNN clusters. Marker genes for cell types were obtained from Haber et. al. for intestinal cell types and Halpern et.al. for liver cell types. Module scores were also created for selected pathway gene lists, such as KEGG_PPAR in the same way.

To gain further resolution in the analysis, Applicants performed iterative clustering. In very large datasets cell types or subtypes which are small in number compared to the total often do not drive enough of the total variation to clearly cluster out by SNN. By subsetting the data to include only a smaller selection of cells, Applicants increased resolution to call more subtle differences or identify rarer cell types within this subset group as variation driven by the small group of cells is now enough of the total to separate clearly by SNN. Applicants performed iterative clustering over groups selected by sample of origin, and cell type and cluster(s).

Pathway analysis was performed using Ingenuity Pathway Analysis (IPA) from Qiagen on selected differentially expressed gene lists to identify biological processes which may vary between the compared groups. DEGs were identified as described above, and filtered to include only genes with a p-adjusted value of <0.1 and an average log-fold change of >0.25 for input to IPA. Core analysis was run using default settings. Interesting IPA results were curated manually.

### Organoid Culture

Hepatocyte organoid culturing was performed as described previously. For organoid Seq-Well experiment, organoids were dissociated to single cell suspension and loaded 15,000 cell per array. On array each was run for organoids from HF3, HF4 and CD4. ATPase growth assay was performed after approximately 2 months in culture. Aspirate media from well, add 65 uL CTG3D (Promega) to each well, seal plate and shake at room temperature 30 minutes. Transfer 15ul to white 384 wp (in triplicate), read at 1 sec lum interval time on luminescence plate reader.

### Statistics

Effect size for expression of module scores was calculated Cohen's d. Significance in the organoid growth assay was calculated using ANOVA with correction for multiple testing.

The following references relate to example 4:
1 Beyaz, S. et al. High-fat diet enhances stemness and tumorigenicity of intestinal progenitors. Nature 531, 53-58, doi:10.1038/nature17173 (2016).
2 Cordain, L. et al. Origins and evolution of the Western diet: health implications for the 21st century. Am J Clin Nutr 81, 341-354, doi:10.1093/ajcn.81.2.341 (2005).
3 Ramachandran, A. & Snehalatha, C. Rising burden of obesity in Asia. J Obes 2010, doi:10.1155/2010/868573 (2010).
4 Kain, J., Uauy, R., Vio, F. & Albala, C. Trends in overweight and obesity prevalence in Chilean children: comparison of three definitions. Eur J Clin Nutr 56, 200-204, doi:10.1038/sj.ejcn.1601301 (2002).
5 Vandegrift, D. & Yoked, T. Obesity rates, income, and suburban sprawl: an analysis of US states. Health Place 10, 221-229, doi:10.1016/j.healthplace.2003.09.003 (2004).
6 Mihaylova, M. M., Sabatini, D. M. & Yilmaz, O. H. Dietary and metabolic control of stem cell function in physiology and cancer. Cell Stem Cell 14, 292-305, doi:10.1016/j.stem.2014.02.008 (2014).
7 Friedman, S. L., Neuschwander-Tetri, B. A., Rinella, M. & Sanyal, A. J. Mechanisms of NAFLD development and therapeutic strategies. Nat Med 24, 908-922, doi:10.1038/s41591-018-0104-9 (2018).
8 Pinte, L., Balaban, D. V., Baicus, C. & Jinga, M. Non-alcoholic fatty pancreas disease—practices for clinicians. Rom J Intern Med, doi:10.2478/rjim-2019-0005 (2019).
9 Argo, C. K. & Caldwell, S. H. Epidemiology and natural history of non-alcoholic steatohepatitis. Clin Liver Dis 13, 511-531, doi:10.1016/j.cld.2009.07.005 (2009).
10 El-Serag, H. B. Hepatocellular carcinoma: recent trends in the United States. Gastroenterology 127, S27-34 (2004).
11 Waghray, A., Murali, A. R. & Menon, K. N. Hepatocellular carcinoma: From diagnosis to treatment. World J Hepatol 7, 1020-1029, doi:10.4254/wjh.v7.i8.1020 (2015).
12 Grompe, M. Liver stem cells, where art thou? Cell Stem Cell 15, 257-258, doi:10.1016/j.stem.2014.08.004 (2014).
13 Forbes, S. J. & Newsome, P. N. Liver regeneration—mechanisms and models to clinical application. Nat Rev Gastroenterol Hepatol 13, 473-485, doi: 10.1038/nrgastro.2016.97 (2016).
14 Gilgenkrantz, H. & Collin de l'Hortet, A. Understanding Liver Regeneration: From Mechanisms to Regenerative Medicine. Am J Pathol 188, 1316-1327, doi:10.1016/j.ajpath.2018.03.008 (2018).
15 Raven, A. et al. Cholangiocytes act as facultative liver stem cells during impaired hepatocyte regeneration. Nature 547, 350-354, doi: 10.1038/nature23015 (2017).
16 Gierahn, T. M. et al. Seq-Well: portable, low-cost RNA sequencing of single cells at high throughput. Nat Methods 14, 395-398, doi:10.1038/nmeth.4179 (2017).
17 Winzell, M. S. & Ahren, B. The high-fat diet-fed mouse: a model for studying mechanisms and treatment of impaired glucose tolerance and type 2 diabetes. Diabetes 53 Suppl 3, S215-219 (2004).
18 MacParland, S. A. et al. Single cell RNA sequencing of human liver reveals distinct intrahepatic macrophage populations. Nat Commun 9, 4383, doi: 10.1038/s41467-018-06318-7 (2018).
19 Weibel, E. R., Staubli, W., Gnagi, H. R. & Hess, F. A. Correlated morphometric and biochemical studies on the liver cell. I. Morphometric model, stereologic methods, and normal morphometric data for rat liver. J Cell Biol 42, 68-91 (1969).
20 Institute, B. GSEA MSigDB, <http://software.broadinstitute.org/gsea/msigdb>(2019).
21 Dyson, N. J. RB1: a prototype tumor suppressor and an enigma. Genes Dev 30, 1492-1502, doi:10.1101/gad.282145.116 (2016).
22 Ricca, A. et al. relA over-expression reduces tumorigenicity and activates apoptosis in human cancer cells. Br J Cancer 85, 1914-1921, doi:10.1054/bjoc.2001.2174 (2001).
23 Madison, B. B. Srebp2: A master regulator of sterol and fatty acid synthesis. J Lipid Res 57, 333-335, doi:10.1194/j1r.0066712 (2016).
24 Reebye, V. et al. Gene activation of CEBPA using saRNA: preclinical studies of the first in human saRNA drug candidate for liver cancer. Oncogene 37, 3216-3228, doi:10.1038/s41388-018-0126-2 (2018).
25 Fozzatti, L. et al. Resistance to thyroid hormone is modulated in vivo by the nuclear receptor corepressor (NCOR1). Proc Natl Acad Sci USA 108, 17462-17467, doi:10.1073/pnas.1107474108 (2011).
26 Mottis, A., Mouchiroud, L. & Auwerx, J. Emerging roles of the corepressors NCoR1 and SMRT in homeostasis. Genes Dev 27, 819-835, doi:10.1101/gad.214023.113 (2013).
27 Hu, H. et al. Long-Term Expansion of Functional Mouse and Human Hepatocytes as 3D Organoids. Cell 175, 1591-1606 e1519, doi:10.1016/j.cell.2018.11.013 (2018).
28 Young, M. B., S. SoupX removes ambient RNA contamination from droplet based single cell RNA sequencing data. bioRxiv (2018).
29 Haber, A. L. et al. A single-cell survey of the small intestinal epithelium. Nature 551, 333-339, doi:10.1038/nature24489 (2017).
30 Halpern, K. B. et al. Paired-cell sequencing enables spatial gene expression mapping of liver endothelial cells. Nat Biotechnol 36, 962-970, doi:10.1038/nbt.4231 (2018).

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A method of treating liver injury comprising
stimulating functional compensation in liver cells by administering an agent that stimulates macrophage Wnt signaling.

2. The method of claim 1, wherein administering an agent comprises delivering a vector that targets liver macrophages.

3. The method of claim 1, wherein administering an agent comprises delivery of an agent targeting hepatocytes thereby stimulating macrophage Wnt signaling at the site of livery injury.

4. A method of decreasing cancer susceptibility and/or inflammation comprising administering a subject in need thereof an inhibitor of peroxisome proliferator-activated receptors (PPARs).

5. The method of claim 2, wherein the PPAR is alpha, beta/delta or gamma.

6. The method of claim 2, wherein administration of the inhibitor is localized to the gut or localized to the liver.

7. The method of claim 2, wherein the subject is obese or on a high fat diet.

8. A method of reducing risk of proliferation disorders or cancer in the liver comprising administering to a subject in need thereof an agent that increases expression of Sox9 or decreases expression of Lrg5 and Axin 2.

9. A method of treating an injury in an organ or tissue, comprising:
administering to a subject in need thereof an agent that modulates expression and/or activity of one or more genes or gene products that have functions in: regulation of proteolysis, chemical homeostasis, secretion by cells, regulation of hydrolase activity, regulation of body fluid levels, homeostatic process, wound healing, glycerolipid metabolic process, response to external stimuli, response to oxygen containing compounds, response to lipid, neutral lipid metabolic process, negative regulation of hydrolase activity, ion homeostasis, response to biotic stimulus, exocytosis, platelet degranulation, response to alcohol, regulated exocytosis, negative regulation of peptidase activity, extracellular matrix, secretory granule, platelet alpha granule, secretory granule lumen, secretory vesicle, vesicle lumen, blood microparticle, intracellular vesicle, extracellular space, cytoplasmic vesicle part, protein lipid complex, enzyme inhibitor activity, enzyme regulator activity, response to hypoxia, apoptosis, complement components functions and activities, or a combination thereof.

10. The method of claim 1, further comprising:
administering to the subject in need thereof another agent that modulates expression and/or activity of one or more genes or gene products that have functions in PPAR signaling pathway, complement and/or coagulation cascades, PPARa activated gene expression, biological oxidations, metabolism of lipids and lipoproteins, nasopharyngeal carcinoma, intestine probiotics, plasma cell vs plasmablast, liver cancer, liver specific genes, multiple myeloma, response to UVb radiation, heart atrium vs ventricle, aging kidney no blood, endocrine therapy resistance, liver cancer, breast cancer basal, foxa2 targets, stem cell, lung cancer kras, tlx targets, liver cancer subclass g123, liver cancer subclass proliferation, liver cancer stem cell, liver cancer recurrence, liver cancer subclass s3, hepatoblastoma, liver development, liver hnf1a targets, matrisome, liver cancer krt19, fatty acid catabolic process, ammonium ion metabolic process, protein activation cascade, regulation of wound healing, response to estradiol, response to acid chemical, sterol homeostasis, lipoprotein metabolic process, fatty acid beta oxidation, protein maturation, regulation of locomotion, organic hydroxy compound metabolic process, organic acid biosynthetic process, monocarboxylic acid metabolic process, response to inorganic substance, regulation of vesicle mediated transport, regulation of fatty acid metabolic process, organic hydroxy compound transport, defense response, organophosphate ester transport, lipid homeostasis, secretion, anion transport, regulation of lipid biosynthetic process, response to xenobiotic stimulus, regulation of response to external stimulus, small molecule biosynthetic process, regulation of response to external stimulus, regulation of lipid metabolic process, amine metabolic process, autophagy, regulation of secretion, apoptotic signaling pathway, acute inflammatory response, regulation of catabolic process, maintenance of location, regulation of protein secretion, organic acid metabolic process, response to oxygen levels, regulation of cellular ketone metabolic process, organic acid catabolic process, regulation of response to wounding, regulation of extrinsic apoptotic signaling pathway, cellular lipid catabolic process, regulation of reactive oxygen species metabolic process, detoxification, regulation of peptidase activity, organic anion transport, inflammatory response, negative regulation of cell death, fatty acid metabolic process, lipid metabolic process, divalent inorganic cation homeostasis, regulation of endocytosis, alcohol metabolic process, immune response, cellular lipid metabolic process, monocarboxylic acid transport, negative regulation of apoptotic signaling pathway, multicellular organismal homeostasis, organic hydroxy compound biosynthetic process, regulation of cell death, lipid catabolic process, regulation of lipid metabolic process, regulation of steroid metabolic process, regulation of inflammatory response, response to toxic substance, cellular chemical homeostasis, regulation of transport, regulation of lipid catabolic process, regulation of immune effector process, lipid localization, regulation of proteolysis, regulation of secretion, regulation of response to wounding, regulation of multicellular organismal process, cellular homeostasis, single organism catabolic process, response to oxidative stress, behavior, acute phase response, regulation of response to external stimulus, regulation of apoptotic signaling pathway, regulation of cell proliferation, response to reactive oxygen species, endocytic vesicle, endoplasmic reticulum part, endoplasmic reticulum lumen, endoplasmic reticulum, lipid transporter activity, sulfur compound binding, steroid binding, glycosaminoglycan binding, alcohol binding, carboxylic ester hydrolase activity, lipid binding, receptor binding, coenzyme binding, adipogenesis, xenobiotic metabolism, fatty acid metabolism, coagulation, bile acid metabolism, peroxisome, or a combination thereof.

3. The method of claim 1, further comprising administering to the subject in need thereof another agent that modulates expression and/or activity of one or more genes or gene products that have functions in HDAC3 targets, photodynamic therapy stress, CEBP targets, tolerant macrophage, response to salirasib, adult tissue stem module, klfl targets, anatomical structure formation involved in morphogenesis, circulatory system process, cellular response to external stimulus, response to wounding, cellular response to extracellular stimulus, cell activation, cellular response to oxygen containing compound, vesicle mediated transport, enzyme linked receptor protein signaling pathway, response to bacterium, regulation of catabolic process, response to ketone, regulation of cell adhesion, response to hormone, blood vessel morphogenesis, response to estrogen, response to radiation, response to extracellular stimulus, cellular response to nitrogen compound, regulation of catalytic activity, vasculature development, response to abiotic stimulus, response to drug, response to growth factor, regulation of protein metabolic process, transmembrane receptor protein tyrosine kinase signaling pathway, cellular response to peptide, hexose metabolic process, cellular response to stress, endocytosis, circulatory system development, response to starvation, hemostasis, response to molecule of bacterial origin, cell surface, peptidase regulator activity, molecular function regulator, peptidase inhibitor activity, phospholipid binding, TNF-a signaling via NFkB, or a combination thereof.

4. The method of claim 1, wherein the agent modulates expression and/or activity of one or more genes or gene products in Wnt pathway.

5. The method of claim 1, wherein the agent modulates expression and/or activity of one or more genes or gene products that are markers of hepatic stem cells.

6. The method of claim 1, wherein the expression and/or activity of the one or more genes or gene products is altered both in response to a zone-dependent injury and a zone-independent injury.

7. The method of claim 1, wherein the one or more genes or gene products comprises Gclc, Txnrd1, Lars2, Cyp4a14, Apoc2, Apoc1, Cyp2c29, Mt1, Mt2, Saa1, Saa2, Fgl1, Mup17, Mup18, Mup11, Gm23935, mmu-mir-6236, Ly6e, Rnase4, Saa4, Fgl1, Hp, Hpx, Lcn2, Orm1, Apcs, Orm2, Saa1, Saa2, Saa3, Sds, Tacc2, Igfbp1, Cxc11, Thrsp, Serpina3n, Lpin1, Steap4, Mt1, Mt2, Aldh3a2, Cyp2c37, Cyp2c29, Cyp8b1, Ces1d, Apoc1, Hsd17b13, Atp5h, Apoc2, Retsat, Mat1a, Angptl3, Chchd10, Hmgcs2, Cyp4a10, Cyp4a14, Gm26917, Lars2, Hyou1, Arrdc3, Mup12, Gm15564, Pdia3, Gm26924, Sephs2, Grip2, Krt8, Krt18, Plin2, Chka, Gclc, Srxn1, Hmox1, S100a8, S100a9, Mup15, Mup4, Ankrd55, Mup11, Mup5, Mup18, Mup9, Mup6, Mup17, Mup19, Alb, Pck1, Slc2a2, F2, Cyp2e1, Glu1, Arg1, Cdh1, Gls2, Ppargc1a, Sox9, Tbx3, Lgr5, Axin2, or a combination thereof.

8. The method of claim 1, wherein the one or more genes or gene products are selected from the genes or gene products in any one of Tables 1a-8c or in all of Tables 1a-8c.

9. The method of claim 1, wherein the agent induces regeneration and/or functional compensation of the organ or tissue.

10. The method of claim 1, wherein the agent induces generation of cells that compensate for function loss caused by the injury in the organ or tissue.

11. The method of claim 1, wherein the agent induces cell proliferation in the organ or tissue.

12. The method of claim 1, wherein the organ or tissue is liver, spleen, intestine, colon, bone marrow, an immune tissue or organ, or a tissue or organ of the gastrointestinal tract.

13. The method of claim 1, wherein the injury is an acute injury.

14. The method of claim 1, wherein the injury is a chronic injury.

15. The method of claim 1, wherein the injury is caused by a metabolic or toxic insult.

16. The method of claim 1, wherein the injury is caused by high fat diet.

17. The method of claim 1, wherein the injury is caused by a disease.

18. The method of claim 1, wherein the injury is caused by a chronic disease.

19. The method of claim 1, wherein the disease is a liver disease.

20. The method of claim 19, wherein the liver disease is non-alcoholic fatty liver disease, non-alcoholic steatohepatitis, or cirrhosis.

21. The method of claim 1, wherein the injury is a zone-independent injury.

22. The method of claim 1, wherein the injury is a zone-dependent injury.

23. The method of claim 1, wherein the agent is a CRISPR-Cas agent.

24. A method of treating an injury in an organ or tissue, comprising:
a. determining expression of one or more genes from single cells in the organ or tissue at a first time point and a second time point;
b. selecting a first subset of genes from the one or more genes, wherein expression of the first subset of genes at the first and the second time points are different;
c. determining spatial locations of cells expressing the first subset of genes in the organ or tissue at the first and the second time points by an in situ hybridization assay;
d. selecting a second subset of genes based on the spatial locations of the cells expressing the second subset of genes; and
e. administering an agent that modulates expression and/or activity of one or more of the second subset of genes to a subject in need thereof.
